(19) United States
(12) Patent Application Publication
(10) Pub. No.: US 2025/0387504 A1
(43) Pub. Date: Dec. 25, 2025

(54) ANTIBODY-DRUG CONJUGATES OF ANTINEOPLASTIC COMPOUNDS AND METHODS OF USE THEREOF

(71) Applicants: Novartis AG, Basel (CH); Les Laboratoires Servier, Suresnes Cedex (FR)

(72) Inventors: Matthew T. Burger, Belmont, MA (US); Zhuoliang Chen, Belmont, MA (US); Joseph Anthony D'Alessio, Cambridge, MA (US); Gregory John Hollingworth, Basel (CH); Claudia Judith Klinter, Basel-stadt (CH); Jean-Baptiste Langlois, Schlierbach (FR); Eric Andrew McNeill, Arlington, MA (US); Cornelia Anne Mundt, Lorrach (DE); Katsumasa Nakajima, Winchester, MA (US); Richard Vaughan Newcombe, Acton, MA (US); Katharina Madörin, Basel (CH); Bing Yu, Belmont, MA (US); Qiang Zhang, East Brunswick, NJ (US); Imre Fejes, Budapest (HU); Olivier Geneste, Rueil-Malmaison (FR); Ana Leticia Maragno, Saint-Germain en Laye (FR); Stuart Ray, Cottenham (GB); Francesca Rocchetti, Vienna (AT); Jérôme-Benoit Starck, Rueil-Malmaison (FR); Zoltán Szlavik, Szentendre (HU); Vesela Kostova, Orsay (FR); Tibor Novák, Budapest (HU)

(21) Appl. No.: 18/866,153

(22) PCT Filed: May 19, 2023
(86) PCT No.: PCT/US2023/022990

§ 371 Date: Nov. 15, 2024

Related U.S. Application Data
(60) Provisional application No. 63/344,510, filed on May 20, 2022.

(51) Int. Cl. A61K 47/68; A61P 35/00; C07K 16/28; C07K 16/32

(52) U.S. Cl. CPC A61K 47/6849; A61K 47/68031; A61K 47/68037; A61K 47/6855; A61K 47/6889; A61P 35/00; C07K 16/2833; C07K 16/2863; C07K 16/2866; C07K 16/32

(57) ABSTRACT

Antibody-drug conjugates that bind to human oncology targets are disclosed. The antibody-drug conjugates comprise an antibody or an antigen-binding fragment thereof covalently linked to at least one BH3 mimetic through a dual linker. The disclosure further relates to methods and compositions for use in the treatment of cancers by administering the antibody-drug conjugates provided herein. Linker-drug conjugates comprising at least one BH3 mimetic and methods of making the same are also disclosed.

### RELATED APPLICATION

This application claims the benefit of the filing date, under 35 U.S.C. § 119(e), of U.S. Provisional Application No. 63/344,510, filed on May 20, 2022, the entire contents of which are incorporated here by reference.

### FIELD OF THE INVENTION

The present disclosure relates to antibody-drug conjugates (ADCs) comprising an antibody or an antigen-binding fragment thereof covalently linked to two antineoplastic compounds through a dual linker, wherein at least one antineoplastic payload is a BH3 mimetic. The disclosure further relates to methods and compositions useful in the treatment and/or diagnosis of cancers that express a target antigen and/or are amenable to treatment by modulating expression and/or activity of Bcl-2 family proteins, as well as methods of making those compositions. Linker-drug conjugates comprising the dual linker and antineoplastic compounds (e.g., two BH3 mimetics or a BH3 mimetic moiety and an antineoplastic non-BH3 mimetic) and methods of making same are also disclosed.

### BACKGROUND OF THE INVENTION

Apoptosis (programmed cell death) is an evolutionarily conserved pathway essential for tissue homeostasis, development and removal of damaged cells. Deregulation of apoptosis contributes to human diseases, including malignancies, neurodegenerative disorders, diseases of the immune system and autoimmune diseases (Hanahan and Weinberg, Cell. 2011 Mar. 4; 144(5):646-74; Marsden and Strasser, Annu Rev Immunol. 2003; 21:71-105; Vaux and Flavell, Curr Opin Immunol. 2000 December; 12(6):719-24). Evasion of apoptosis is recognized as a hallmark of cancer, participating in the development as well as the sustained expansion of tumors and the resistance to anti-cancer treatments (Hanahan and Weinberg, Cell. 2000 Jan. 7; 100(1):57-70).

The Bcl-2 protein family comprises key regulators of cell survival which can suppress (e.g., Bcl-2, Bcl-xL, Mcl-1) or promote (e.g., Bad, Bax) apoptosis (Gross et al., Genes Dev. 1999 Aug. 1; 13(15):1899-911, Youle and Strasser, Nat. Rev. Mol. Cell Biol. 2008 January;9(1):47-59).

In the face of stress stimuli, whether a cell survives or undergoes apoptosis is dependent on the extent of pairing between the Bcl-2 family members that promote cell death with family members that promote cell survival. For the most part, these interactions involve the docking of the Bcl-2 homology 3 (BH3) domain of proapoptotic family members into a groove on the surface of pro-survival members. The presence of Bcl-2 homology (BH) domain defines the membership of the Bcl-2 family, which is divided into three main groups depending upon the particular BH domains present within the protein. The prosurvival members such as Bcl-2, Bcl-xL, and Mcl-1 contain BH domains 1-4, whereas Bax and Bak, the proapoptotic effectors of mitochondrial outer membrane permeabilization during apoptosis, contain BH domains 1-3 (Youle and Strasser, Nat. Rev. Mol. Cell Biol. 2008 January;9(1):47-59).

Overexpression of the prosurvival members of the Bcl-2 family is a hallmark of cancer and it has been shown that these proteins play an important role in tumor development, maintenance and resistance to anticancer therapy (Czabotar et al., Nat. Rev. Mol. Cell Biol. 2014 January;15(1):49-63). Bcl-xL (also named BCL2L1, from BCL2-like 1) is frequently amplified in cancer (Beroukhim et al., Nature 2010 Feb 18;463(7283):899-905) and it has been shown that its expression inversely correlates with sensitivity to more than 120 anti-cancer therapeutic molecules in a representative panel of cancer cell lines (NCI-60) (Amundson et al., Cancer Res. 2000 Nov. 1; 60(21):6101-10).

In addition, several studies using transgenic knockout mouse models and transgenic overexpression of Bcl-2 family members highlighted the importance of these proteins in the diseases of the immune system and autoimmune diseases (for a review, see Merino et al., Apoptosis 2009 Apr;14(4):570-83. doi: 10.1007/s10495-008-0308-4.PMID: 19172396). Transgenic overexpression of Bcl-xL within the T-cell compartment resulted in resistance to apoptosis induced by glucocorticoid, g-radiation and CD3 crosslinking, suggesting that transgenic Bcl-xL overexpression can reduce apoptosis in resting and activated T-cells (Droin et al., Biochim Biophys Acta 2004 Mar 1;1644(2-3):179-88. doi: 10.1016/j.bbamcr.2003.10.011.PMID: 14996502). In patient samples, persistent or high expression of antiapoptotic Bcl-2 family proteins has been observed (Pope et al., Nat Rev Immunol. 2002 July;2(7):527-35. doi: 10.1038/nri846.PMID: 12094227). In particular, T-cells isolated from the joints of rheumatoid arthritis patients exhibited increased Bcl-xL expression and were resistant to spontaneous apoptosis (Salmon et al., J Clin Invest. 1997 Feb. 1; 99(3):439-46. doi: 10.1172/JCI119178.PMID: 9022077).

The findings indicated above motivated the discovery and development of a new class of drugs named BH3 mimetics. These molecules are able to disrupt the interaction between the proapoptotic and antiapoptotic members of the Bcl-2 family and are potent inducers of apoptosis. This new class of drugs includes inhibitors of Bcl-2, Bcl-xL, Bcl-w and Mcl-1. The first BH3 mimetics described were ABT-737 and ABT-263, targeting Bcl-2, Bcl-xL and Bcl-w (Park et al., J. Med. Chem. 2008 Nov. 13; 51(21):6902-15; Roberts et al., J. Clin. Oncol. 2012 Feb. 10; 30(5):488-96). After that, selective inhibitors of Bcl-2 (ABT-199 and S55746 - Souers et al., Nat Med. 2013 February;19(2):202-8; Casara et al., Oncotarget 2018 Apr 13;9(28):20075-20088), Bcl-xL (A-1155463 and A-1331852 - Tao et al., ACS Med Chem Lett. 2014 Aug. 26; 5(10):1088-93; Leverson et al., Sci Transl Med. 2015 Mar. 18; 7(279):279ra40) and Mcl-1 (A-1210477, S63845, S64315, AMG-176 and AZD-5991 - Leverson et al., Cell Death Dis. 2015 Jan. 15; 6:e1590.; Kotschy et al., Nature 2016, 538, 477-482; Maragno et al., AACR 2019, Poster #4482; Kotschy et al., WO 2015/097123; Caenepeel et al., Cancer Discov. 2018 December; 8(12):1582-1597; Tron et al., Nat. Commun. 2018 Dec. 17; 9(1):5341) were also discovered. The selective Bcl-2 inhibitor ABT-199 is now approved for the treatment of patients with CLL and AML in combination therapy, while the other inhibitors are still under pre-clinical or clinical development. In pre-clinical models, ABT-263 has shown activity in several hematological malignancies and solid tumors (Shoemaker et al., Clin. Cancer Res. 2008 Jun. 1; 14(11):3268-77; Ackler et al., Cancer Chemother. Pharmacol. 2010 October;66(5):869-80; Chen et al., Mol. Cancer Ther. 2011 December; 10(12):2340-9). In clinical studies, ABT-263 exhibited objective antitumor activity in lymphoid malignancies (Wilson et al., Lancet Oncol. 2010 December; 11(12):1149-59; Roberts et al., J. Clin. Oncol. 2012 Feb. 10; 30(5):488-96) and its activity is being investigated in combination with several therapies in solid tumors. The selective Bcl-xL inhibitors, A-1155463 or A-1331852, exhibited in vivo activity in pre-clinical models of T-ALL (T-cell Acute Lymphoblastic Leukemia) and different types of solid tumors (Tao et al., ACS Med. Chem. Lett. 2014 Aug. 26; 5(10):1088-93; Leverson et al., Sci. Transl. Med. 2015 Mar. 18; 7(279):279ra40). The use of BH3 mimetics has also shown benefit in pre-clinical models of diseases of the immune system and autoimmune diseases. Treatment with ABT-737 (Bcl-2, Bcl-xL, and Bcl-w inhibitor) resulted in potent inhibition of lymphocyte proliferation in vitro. Importantly, mice treated with ABT-737 in animal models of arthritis and lupus showed a significant decrease in disease severity (Bardwell et al., J Clin Invest. 1997 Feb. 1; 99(3):439-46. doi: 10.1172/JCI119178.PMID: 9022077). In addition, it has been shown that ABT-737 prevented allogeneic T-cell activation, proliferation, and cytotoxicity in vitro and inhibited allogeneic T- and B-cell responses after skin transplantation with high selectivity for lymphoid cells (Cippa et al., .Transpl Int. 2011 July;24(7):722-32. doi: 10.1111/j.1432-2277.2011.01272.x. Epub 2011 May 25.PMID: 21615547).

In pre-clinical studies, it has been shown that BH3 mimetics strongly synergize when in combination, including Mclli+Bcl2i, Mclli+Bcl-xli, Bcl-xli+Bcl-2i (WO 2018015526A1; Moujalled et al., Leukemia. 2019 April; 33(4):905-917; Moujalled et al., Blood Adv. 2020 Jun. 23; 4(12):2762-2767; Grundy et al., Oncotarget. 2018 Dec. 28; 9(102):37777-37789; Soderquist et al., Nat Commun. 2018 Aug. 29; 9(1):3513; Weeden et al., Oncogene. 2018 August; 37(32):4475-4488; Sarah Kehr et al., Cancer Lett. 2020 Jul. 10; 482:19-32). Furthermore, it has also been shown that Bcl-xl inhibitors and Mcli inhibitors strongly synergize when in combination with taxane (Leverson et al, Science Translation Medicine, 2015 March 18; Vol 7(279) 279ra40; Bah et al, Cell Death and Disease, 2014 5, e1291; Wong et al, Mol Cancer Ther., 2012 Apr; 11(4) 1026-1035; Bennett et al, Open Biol., 2016 6: 160134; Topham et al, Cancer Cell, 2015 28, 129-140; Nguyen et al, Clin Cancer Res, 2011 March 15, 17(6) 1394-1404; Merino et al, Science Translational Medicine, 2017 Aug 2;9(401):eaam7049) or when in combination with topoisomerase 1 inhibitors (Scherr et al, Cell Death and Disease, 2020 11:875; Hayward et al, Clin CancerRes 2003 Jul;9(7):2856-65; Lalazar et al, Cancer Discov. 2021 October;11(10):2544-2563; Tolcher et al, Cancer Chemotherapy and Pharmacology, 2015 76,1041-1049). Even if the activity of these combinations is very promising, evidence of tolerance of the administration of two non-conjugated BH3 mimetics in combination or a BH3 mimetic and an antineoplastic non-BH3 mimetic in combination is still missing, in particular for Mclli+Bclxli. Also, the clinical potential of non-conjugated BH3 mimetics combinations is still to be demonstrated. Therefore, there is need to find disease-modifying agents therapeutically targeting Bcl-2 family proteins (e.g., Bcl-2, Bcl-xL, Mcl-1) or upstream and/or downstream proteins in an apoptotic signaling pathway in oncology and in the field of immune and autoimmune diseases.

### SUMMARY OF THE INVENTION

In a first embodiment, the present disclosure provides an antibody-drug conjugate comprising an antibody or an antigen-binding fragment thereof covalently linked to two antineoplastic payloads through a dual linker, wherein at least one antineoplastic payloads is a BH3 mimetic, and wherein the dual linker has one attachment point connected to the antibody and two attachment points to the two antineoplastic payloads and wherein the two antineoplastic payloads can be the same or different. In some embodiments, one antineoplastic payload is a BH3 mimetic and the other antineoplastic payload is an antineoplastic non-BH3 mimetic. In some embodiments, the antineoplastic non-BH3 mimetic is a topoisomerase 1 inhibitor or an anti-mitotic drug. In some embodiments, the topoisomerase 1 inhibitor is selected from topotecan, exatecan, deruxtecan and SN-38. In some embodiments, the anti-mitotic drug is monomethyl auristatin E (MMAE) or a taxane. In some embodiments, the taxane is selected from docetaxel, paclitaxel, or cabazitaxel. In some embodiments, said two antineoplastic payloads are two BH3 mimetics. In some embodiments, the BH3 mimetic is selected from a Mcl-1 inhibitor, a Bcl-2 inhibitor, and a Bcl-xL inhibitor. In some embodiments, the BH3 mimetic of said two antineoplastic payloads are the same. In some embodiments, the BH3 mimetic of said two antineoplastic payloads are different. In some embodiment, the antineoplastic payloads in the antibody-drug conjugate of the present disclosure are defined as: (i) one antineoplastic payload is a Mcl-1 inhibitor and the other antineoplastic payload is a Bcl-2 inhibitor; (ii) one antineoplastic payload is a Mcl-1 inhibitor and the other antineoplastic payload is a Bcl-xL inhibitor; or (iii) one antineoplastic payload is a Bcl-2 inhibitor and the other antineoplastic payload is a Bcl-xL inhibitor. In some embodiments, one antineoplastic payload is a Mcl-1 inhibitor, a Bcl-2 inhibitor, and a Bcl-xL inhibitor, and the other antineoplastic payload is a topoisomerase 1 inhibitor or an anti-mitotic drug. In some embodiments, one antineoplastic payload is a Bcl-xL inhibitor and the other antineoplastic payload is a topoisomerase 1 inhibitor. In some embodiments, one antineoplastic payload is a Bcl-xL inhibitor and the other antineoplastic payload is an anti-mitotic drug. In some embodiments, one antineoplastic payload is a Mcl-1 inhibitor and the other antineoplastic payload is a topoisomerase 1 inhibitor. In some embodiments, one antineoplastic payload is a Mcl-1 inhibitor and the other antineoplastic payload is an anti-mitotic drug. In some embodiments, one antineoplastic payload is a Bcl-2 inhibitor and the other antineoplastic payload is a topoisomerase 1 inhibitor. In some embodiments, one antineoplastic payload is a Bcl-2 inhibitor and the other antineoplastic payload is an anti-mitotic drug.

In a second embodiment, the present disclosure provides antibody-drug conjugate of the first embodiment, wherein the antibody-drug conjugate is represented by Formula (A):

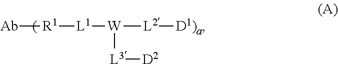

wherein:
Ab is an antibody or an antigen-binding fragment thereof;
R¹ is an attachment group;
L¹ is a bridging spacer;
W is branching moiety;
L^{2′} and L^{3′}, are each independently a linker;
D¹ and D² are each independently an antineoplastic compound, wherein at least one of D¹ and D²is a BH3 mimetic; and
a is an integer from 1 to 16. In some embodiments, D¹ and D² are each independently a BH3 mimetic.

In a third embodiment, the present disclosure provides an antibody-drug conjugate of the second embodiment, wherein a is an integer from 1 to 8, 1 to 6, 1 to 4, or a is 1 or 2, optionally wherein a is determined by liquid chromatography-mass spectrometry (LC-MS). The definitions of the remaining variables are provided in the second embodiment or any embodiments described therein. In some embodiments, a is an integer from 1 to 6 or from 1 to 4 or a is 1 or 2 or a is determined by liquid chromatography-mass spectrometry (LC-MS).

In a fourth embodiment, the present disclosure provides an antibody-drug conjugate of the second or third embodiment, wherein each of L^{2′} and L³′ comprises a cleavable group, optionally wherein at least one cleavable group comprises a glucuronide group, pyrophosphate group, a peptide group, and/or a self-immolative group. In some embodiments, each of L^{2′} and L³′ comprises a cleavable group, optionally at least one cleavable group comprises a pyrophosphate group, a peptide group and/or a self-immolative group. The definitions of the remaining variables are provided in the second or third embodiment or any embodiments described therein.

In a fifth embodiment, the present disclosure provides an antibody-drug conjugate of the second embodiment, wherein the antibody-drug conjugate is represented by Formula (B):

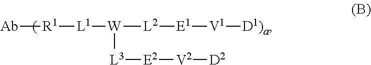

wherein:
Ab is an antibody or an antigen-binding fragment thereof;
R¹ is an attachment group;
L¹ is a bridging spacer;
W is N or CR^{w}; wherein R^{W} is H or C₁₋₆alkyl;
L² and L³ are each independently a connecting spacer;
E¹ and E² are each independently, an enzyme cleavage element or a hydrophilic moiety;
V¹ and V² are each independently comprise: i) a self immolative group, ii) an enzyme
cleavage element, or iii) a self-immolative group and an enzyme cleavage element; and
D¹ and D² are each independently an antineoplastic compound, wherein at least one of D¹ and
D²is a BH3 mimetic.

The definitions of the remaining variables are provided in the second embodiment or any embodiments described therein. In some embodiments, V¹ and V² are each independently i) a self immolative group or ii) an enzyme cleavage element; and D¹ and D² are each independently a BH3 mimetic.

In a sixth embodiment, the present disclosure provides an antibody-drug conjugate of the fifth embodiment, wherein (i) V¹ and V² each independently comprises a phosphate, a pyrophosphate and/or a self-immolative group; (ii) V¹ and V² each independently comprises a self-immolative group; (iii) V¹ and V² each independently comprises a self-immolative group comprising —CH₂—O—, —OC(═O)—, —NH—CH₂—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium; iv) V¹ and V² each independently comprises a group comprising para-aminobenzyl-phosphate or para-aminobenzyl-pyrophosphate. The definitions of the remaining variables are provided in the fifth embodiment or any embodiments described therein.

In some embodiments, for the antibody-drug conjugate of the fifth embodiment, V¹ and V² are defined as: (i) V¹ and V² each independently comprises a phosphate, a pyrophosphate and/or a self-immolative group; (ii) V¹ and V² each independently comprises a self-immolative group; or (iii) V¹ and V² each independently comprises a self-immolative group comprising —CH₂—O—, —OC(═O)—, —NH—CH₂—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.

In a seventh embodiment, the present disclosure provides an antibody-drug conjugate of the fifth embodiment, wherein the antibody-drug conjugate is represented by Formula (C):

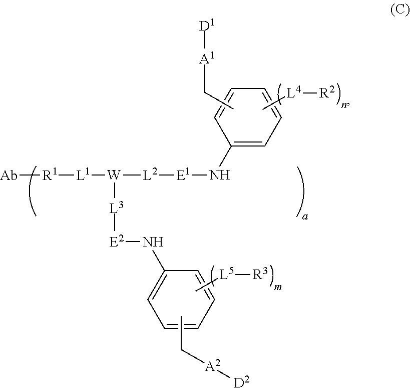

or pharmaceutically acceptable salt thereof, wherein
Ab is an antibody or an antigen-binding fragment thereof;
R¹ is an attachment group;
L¹ is a bridging spacer;
W is N or CR^{w}; wherein R^{W} is H or C_{1 6}alkyl;
L² and L³ are each independently a connecting spacer;
E¹ and E² are each independently a peptide group comprising 1 to 6 amino acids, wherein said peptide group is optionally substituted by a hydrophilic group;
A¹ and A² are each independently a bond, —OC(═O)—*, —OC(═O)NH—*,

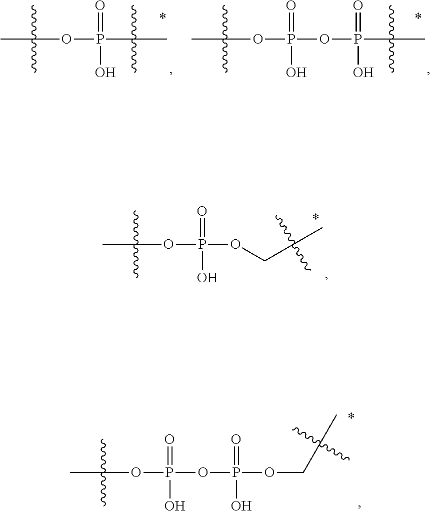 [SMILES: *OP(*)(=O)O ; *OP(=O)(O)OP(*)(=O)O ; *COP(=O)(O)O* ; *COP(=O)(O)OP(=O)(O)O*]

OC(═O)N(CH₃)CH₂CH₂N(CH₃)C(═O)—* or —OC(═O)N(CH₃)C(R^{a})₂C(R^{a})₂N(CH₃)C(═O)—*,
wherein each Ra is independently selected from H, C₁-C₆ alkyl, and C₃-C₅ cycloalkyl and the * of A¹ or A² indicates the point of attachment to D¹ or D²
D¹ and D² are each independently an antineoplastic compound, wherein at least one of D¹ and D² is a BH3 mimetic;
L⁴ and L⁵ are each independently a spacer moiety;
R² and R³ are each independently a hydrophilic group or an enzyme cleavage element; and
m and n are each independently 0 or 1.

The definitions of the remaining variables are provided in the fifth embodiment or any embodiments described therein. In some embodiments, D¹ and D² are each independently a BH3 mimetic.

In an eighth embodiment, the present disclosure provides an antibody-drug conjugate of the seventh embodiment, wherein the antibody-drug conjugate is represented by Formula (D1), (D2), or (D3):

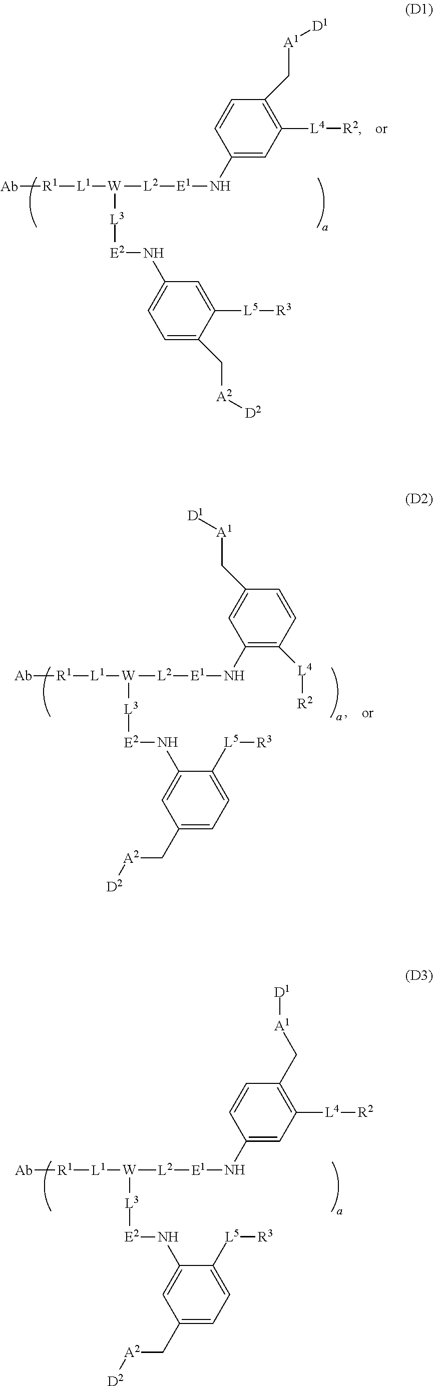

or pharmaceutically acceptable salt thereof, wherein for Formula (D2), D¹ and D² are each independently an antineoplastic compound, wherein at least one of D¹ and D² is a BH3 mimetic; R² and R³ are each independently an enzyme cleavage element; and for Formula (D3), R² is a hydrophilic group and R³ is an enzyme cleavage element. The definitions of the remaining variables are provided in the seventh embodiment or any embodiments described therein. In some embodiments, D, and D² are each independently a BH3 mimetic.

In a ninth embodiment, the present disclosure provides an antibody-drug conjugate of the eighth embodiment, wherein for Formula (D1), R² and R³ are each independently a hydrophilic group. The definitions of the remaining variables are provided in the eighth embodiment or any embodiment described therein.

In a tenth embodiment, the present disclosure provides an antibody-drug conjugate of any one of the second through the ninth embodiments, wherein the attachment group is formed by a reaction comprising at least one reactive group. The definitions of the remaining variables are provided in any one of the second through the ninth embodiments or any embodiment described therein.

In an eleventh embodiment, the present disclosure provides an antibody-drug conjugate of any one of the second through the tenth embodiments, wherein the attachment group is formed by reacting:
a first reactive group that is attached to the linker, and
a second reactive group that is attached to the antibody or is an amino acid residue of the antibody, wherein optionally,

(i) at least one of the reactive groups comprises:
a thiol,
a maleimide,
a haloacetamide,
an azide,
an alkyne,
a cyclcooctene,
a triaryl phosphine,
an oxanobornadiene,
a cyclooctyne,
a diaryl tetrazine,
a monoaryl tetrazine,
a norbornene,
an aldehyde,
a hydroxylamine,
a hydrazine,
NH₂—NH—C(═O)—,
a ketone,
a vinyl sulfone,
an aziridine,
an amino acid residue,

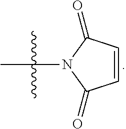 [SMILES: *N1C(=O)C=CC1=O]

—ONH₂, —NH₂,

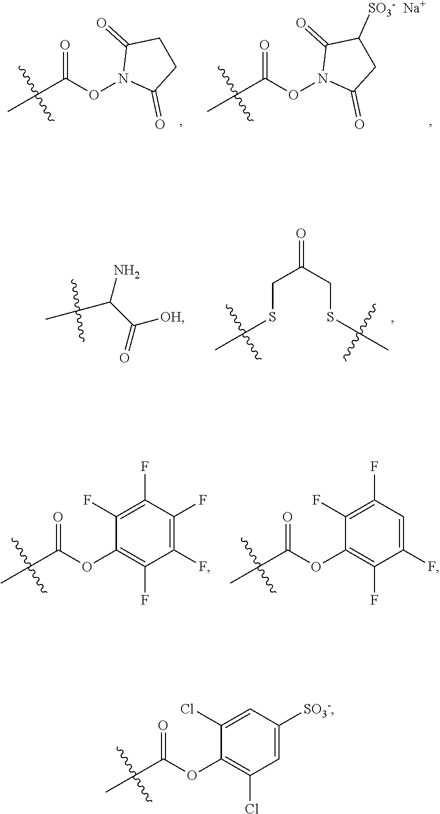

### —N₃,

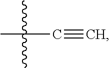 [SMILES: *C#C]

—SH, SR¹¹, SSR¹², —S(═O)₂(CH═CH₂), —(CH₂)₂S(═O)₂(CH═CH₂), —NHS(═O)₂(CH═CH₂), —NHC(═O)CH₂Br, —NHC(═O)CH₂I,

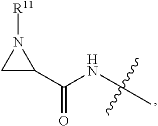

### —C(O)NHNH₂,

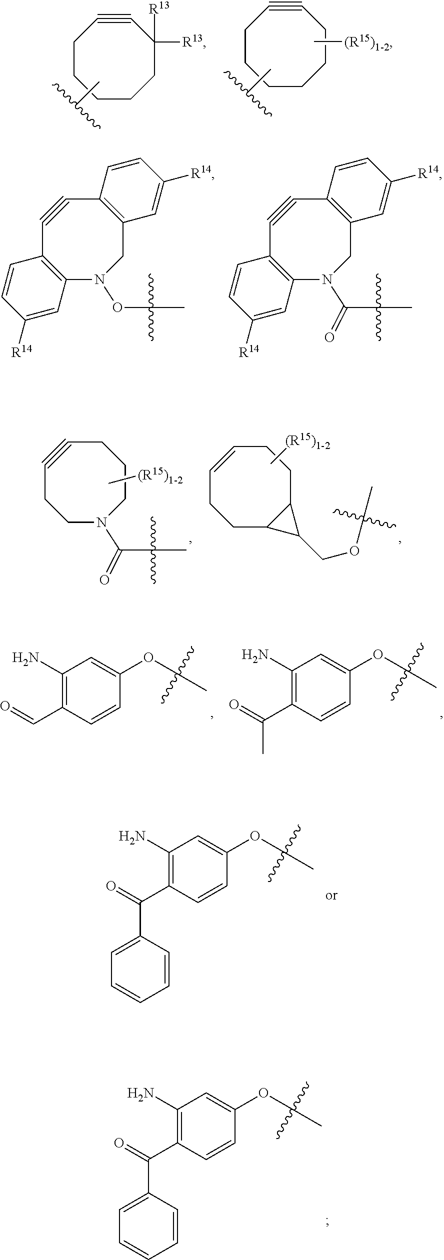

wherein:
each R¹¹ is independently selected from H and C₁-C₆alkyl;
each R¹² is 2-pyridyl or 4-pyridyl;
each R¹³ is independently selected from H, C₁-C₆alkyl, F, Cl, and —OH; each R¹⁴ is independently selected from H, C₁-C₆alkyl, F, Cl, —NH₂, —OCH₃, —OCH₂CH₃, —N(CH₃)₂, —CN, —NO₂ and —OH;
each R¹⁵ is independently selected from H, C_{1 6}alkyl, fluoro, benzyloxy substituted with —C(═O)OH, benzyl substituted with —C(═O)OH, C₁₋₄alkoxy substituted with —C(═O)OH and C_{1 4}alkyl substituted with —C(═O)OH; and/or

(ii) the first reactive group and second reactive group comprise:
a thiol and a maleimide,
a thiol and a haloacetamide,
a thiol and a vinyl sulfone,
a thiol and an aziridine,
an azide and an alkyne,
an azide and a cyclooctyne,
an azide and a cyclooctene,
an azide and a triaryl phosphine,
an azide and an oxanobornadiene,
a diaryl tetrazine and a cyclooctene,
a monoaryl tetrazine and a nonbornene,
an aldehyde and a hydroxylamine,
an aldehyde and a hydrazine,
an aldehyde and NH₂—NH—C(═O)—,
a ketone and a hydroxylamine,
a ketone and a hydrazine,
a ketone and NH₂—NH—C(═O)—,
a hydroxylamine and

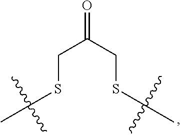 [SMILES: *SCC(=O)CS*]

an amine and

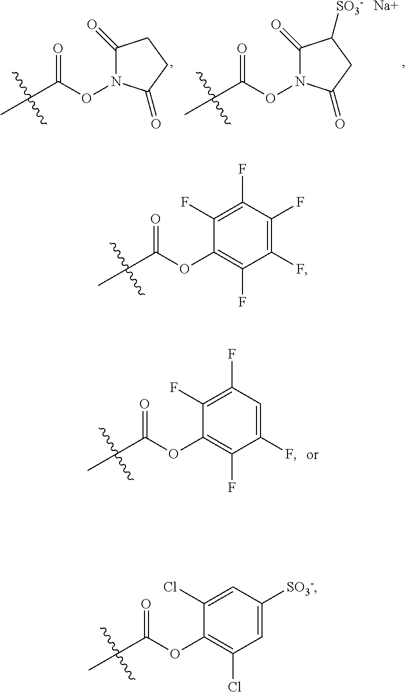

or
a CoA or CoA analogue and a serine residue.

The definitions of the remaining variables are provided in the second through the tenth embodiments or any embodiments described therein.

In a twelfth embodiment, the present disclosure provides an antibody-drug conjugate of any one of the second through the eleventh embodiments, wherein the attachment group is selected from:

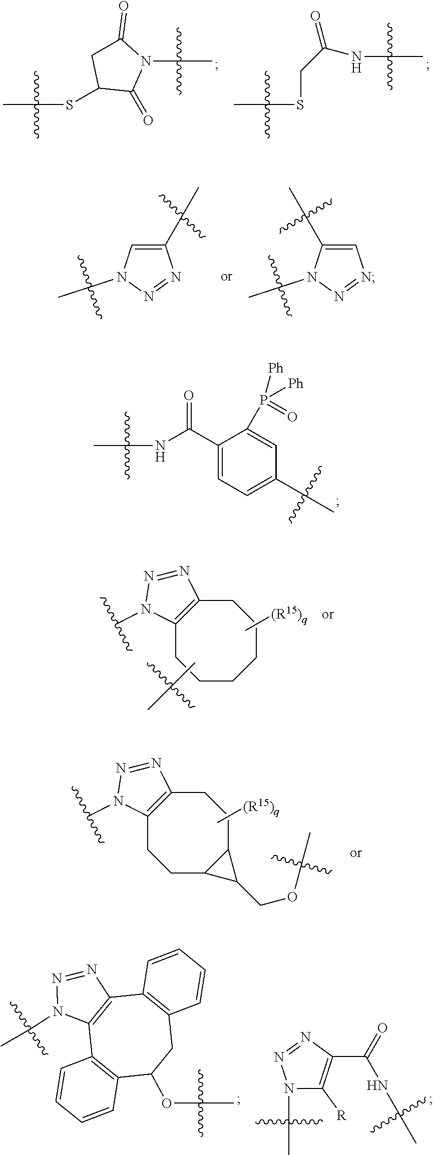

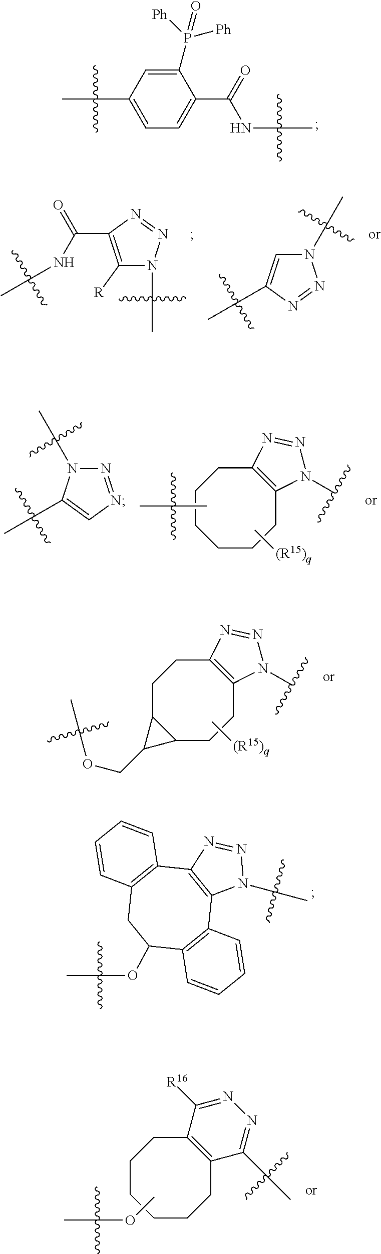

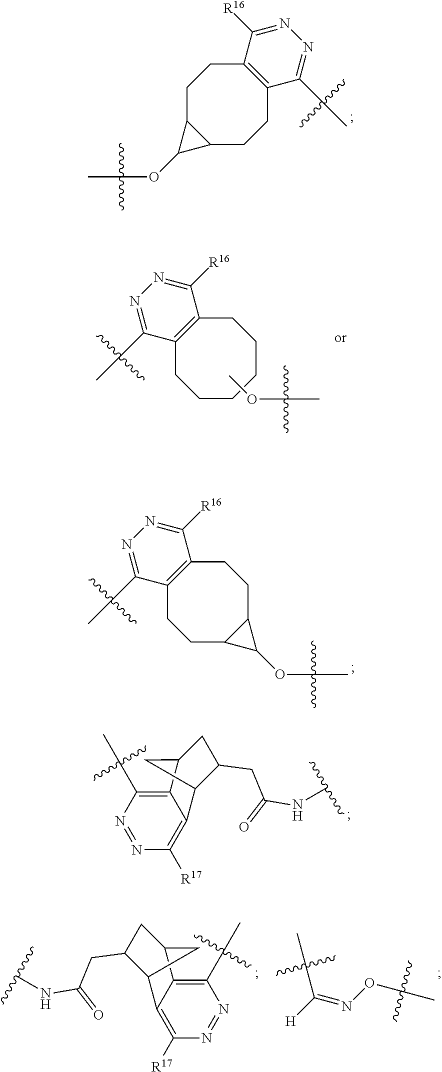

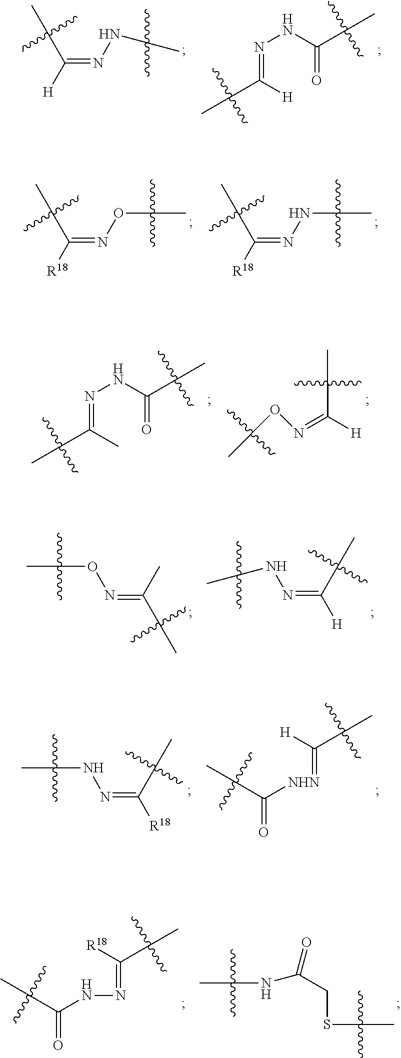

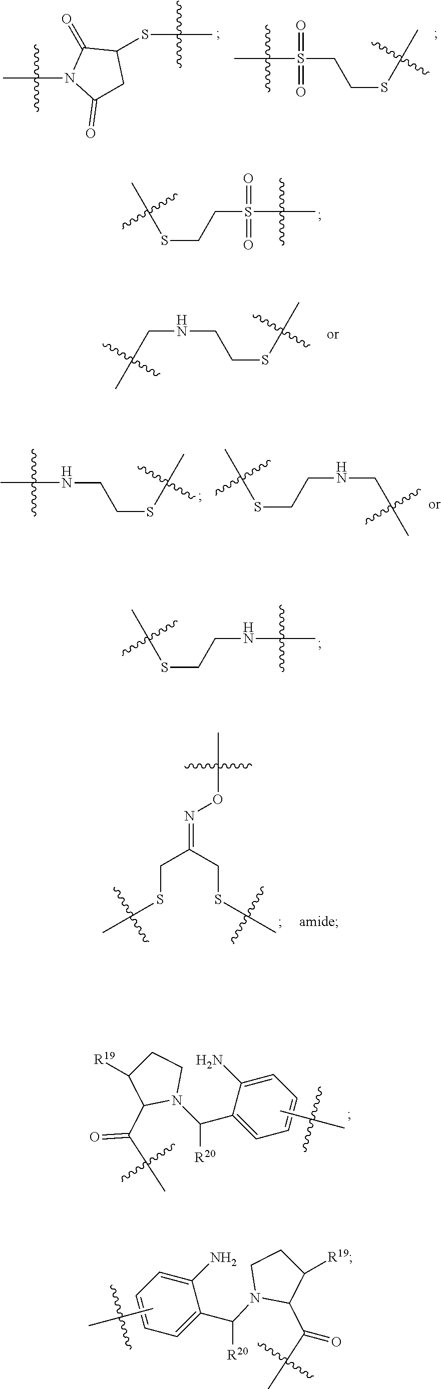

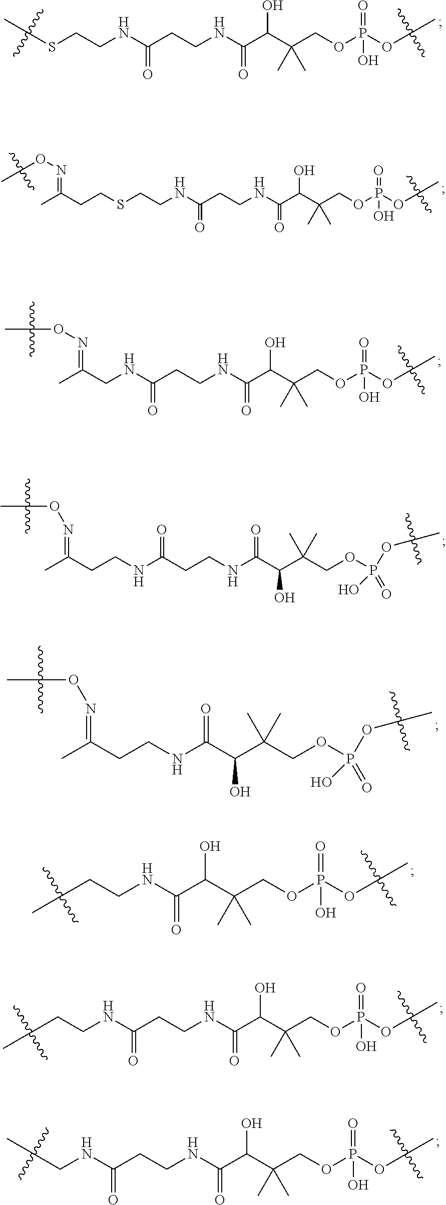

and
disulfide,

wherein:
R¹⁶ is H, C₁₋₄ alkyl, phenyl, pyrimidine or pyridine;
R¹⁸ is H, C₁₋₆ alkyl, phenyl or C_{1 4} alkyl substituted with 1 to 3 —OH groups;
each R¹⁵ is independently selected from H, C₁₋₆ alkyl, fluoro, benzyloxy substituted with —
C(═O)OH, benzyl substituted with —C(═O)OH, C₁₋₄ alkoxy substituted with —C(═O)OH and
C₁₋₄ alkyl substituted with —C(═O)OH;
R¹⁷ is independently selected from H, phenyl and pyridine;
q is 0, 1,2 or 3;
R¹⁹ is H or methyl; and
R²⁰ is H, —CH₃ or phenyl.

The definitions of the remaining variables are provided in the second through the eleventh embodiments or any embodiments described therein.

In a thirteenth embodiment, the present disclosure provides an antibody-drug conjugate of any one of the second through the twelfth embodiments, wherein the attachment group is

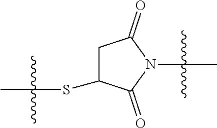 [SMILES: *SC1CC(=O)N(*)C1=O]

The definitions of the remaining variables are provided in the second through the twelfth embodiments or any embodiments described therein.

In a fourteenth embodiment, the present disclosure provides an antibody-drug conjugate of any one of the second through the thirteenth embodiments, wherein:
(1) L¹ comprises:

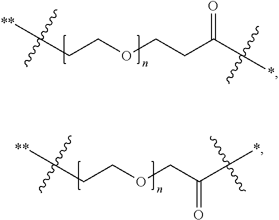

or
*—CH(OH)CH(OH)CH(OH)CH(OH)—**,
wherein each n is an integer from 1 to 12, wherein the * of L¹ indicates the point of direct or indirect attachment to W, and the ** of L¹ indicates the point of direct or indirect attachment to R¹;
(2) L¹ is

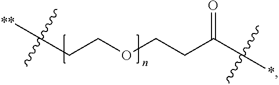

and n is an integer from 1 to 12 or n is 1 or n is 12, wherein the * of L¹ indicates the point of direct or indirect attachment to W, and the ** of L¹ indicates the point of direct or indirect attachment to R¹;
(3) L¹ is

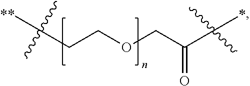

and n is an integer from 1 to 12, wherein the * of L¹ indicates the point of direct or indirect attachment to W, and the ** of L₁ indicates the point of direct or indirect attachment to R¹;
(4) L¹ comprises

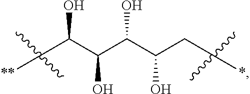

wherein the * of L¹ indicates the point of direct or indirect attachment to W, and the ** of L¹ indicates the point of direct or indirect attachment to R¹;
(5) L¹ is a bridging spacer comprising:
*—C(═O)(CH₂)ₘO(CH₂)ₘ—**; *—C(═O)((CH₂)ₘO)ₜ(CH₂)ₙ—**; *—C(═O)(CH₂)ₘ—**;
*—C(═O)NH((CH₂)ₘO)ₜ(CH₂)ₙ—**;
*—C(═O)O(CH₂)ₘSSC(R^{L1})₂(CH₂)ₘC(═O)NR^{L1}(CH₂)ₘNR^{L1}C(═O)(CH₂)ₘ—**;
*—C(═O)O(CH₂)ₘC(═O)NH(CH₂)ₘ—**; *—C(═O)(CH₂)ₘNH(CH₂)ₘ **;
*—C(═O)(CH₂)ₘNH(CH₂)ₙC(═O)—**; *—C(═O)(CH₂)ₘX₁(CH₂)ₘ—**;
*—C(═O)((CH₂)ₘO)ₜ(CH₂)ₙX₁(CH₂)ₙ—**; *—C(═O)(CH₂)ₘNHC(═O)(CH₂)ₙ **;
*—C(═O)((CH₂)ₘO)ₜ(CH₂)ₙNHC(═O)(CH₂)ₙ—**;
*—C(═O)(CH₂)ₘNHC(═O)(CH₂)ₙX₁(CH₂)ₙ—**;
*—C(═O)((CH₂)ₘO)ₜ(CH₂)ₙNHC(═O)(CH₂)ₙX₁(CH₂)ₙ—**;
*—C(═O)((CH₂)ₘO)ₜ(CH₂)ₙC(═O)NH(CH₂)ₘ—**; *—C(═O)(CH₂)ₘC(R^{L1})₂—** or
*—C(═O)(CH₂)ₘC(═O)NH(CH₂)ₘ—**, wherein the * of L¹ indicates the point of direct or indirect attachment to W, and the ** of L¹ indicates the point of direct or indirect attachment to R¹;
X₁ is

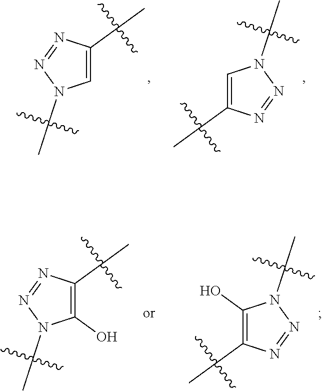 [SMILES: *c1cn(*)nn1 ; *c1cn(*)nn1 ; *c1nnn(*)c1O ; *c1nnn(*)c1O]

and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30; and
each R¹⁷ is independently selected from H and C₁-C₆ alkyl.

The definitions of the remaining variables are provided in the second through the thirteenth embodiments or any embodiments described therein.

In a fifteenth embodiment, the present disclosure provides an antibody-drug conjugate of any one of claims the second through the fourteenth embodiments, wherein L¹ comprises a moiety represented by

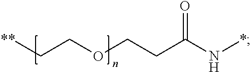

wherein n is an integer from 1 to 12, wherein the * of L¹ indicates the point of direct or indirect attachment to W, and the ** of L¹ indicates the point of direct or indirect attachment to R¹. The definitions of the remaining variables are provided in the second through the fourteenth embodiments or any embodiments described therein.

In a sixteenth embodiment, the present disclosure provides an antibody-drug conjugate of the fifteenth embodiment, wherein L¹ is represented by a formula

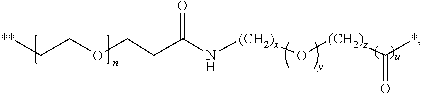

wherein
n is an integer from 1 to 12;
x is an integer from 0 to 6;
y is 0or 1;
z is an integer from 0 to 6;
u is 0 or 1; and
wherein the * of L¹ indicates the point of direct attachment to W, and the ** of L¹ indicates the point of direct attachment to R¹.

The definitions of the remaining variables are provided in the fifteenth embodiment or any embodiments described therein.

In a seventeenth embodiment, the present disclosure provides an antibody-drug conjugate of any one of the second through the sixteenth embodiments, wherein L¹ is selected from the group consisting of:

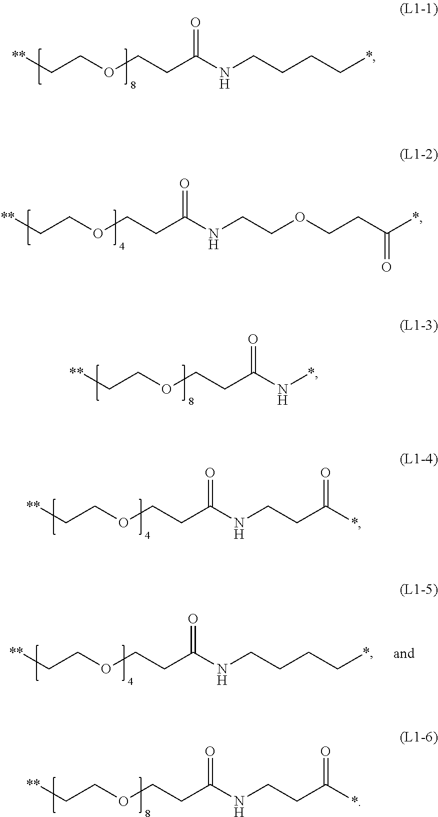

The definitions of the remaining variables are provided in the second through the sixteenth embodiments or any embodiments described therein.

In an eighteenth embodiment, the present disclosure provides an antibody-drug conjugate of any one of claims the fifth through the seventeenth embodiments, wherein L² and L³ are each independently a connecting spacer comprising a moiety represented by:

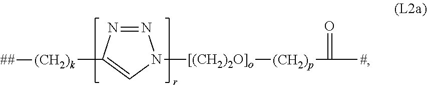

wherein
k is an integer from 0 to 6;
r is0 or 1;
o is an integer from 0 to 12;
p is an integer from 0 to 6; and
wherein the # of L² or L³ indicates the point of direct or indirect attachment to E¹ or E², respectively, and the ## of L² or L³ indicates the point of direct or indirect attachment to W. The definitions of the remaining variables are provided in the second through the seventeenth embodiments or any embodiments described therein.

In a nineteenth embodiment, the present disclosure provides an antibody-drug conjugate of the eighteenth embodiment, wherein L² and L³ are each independently a connecting spacer selected from a group consisting of

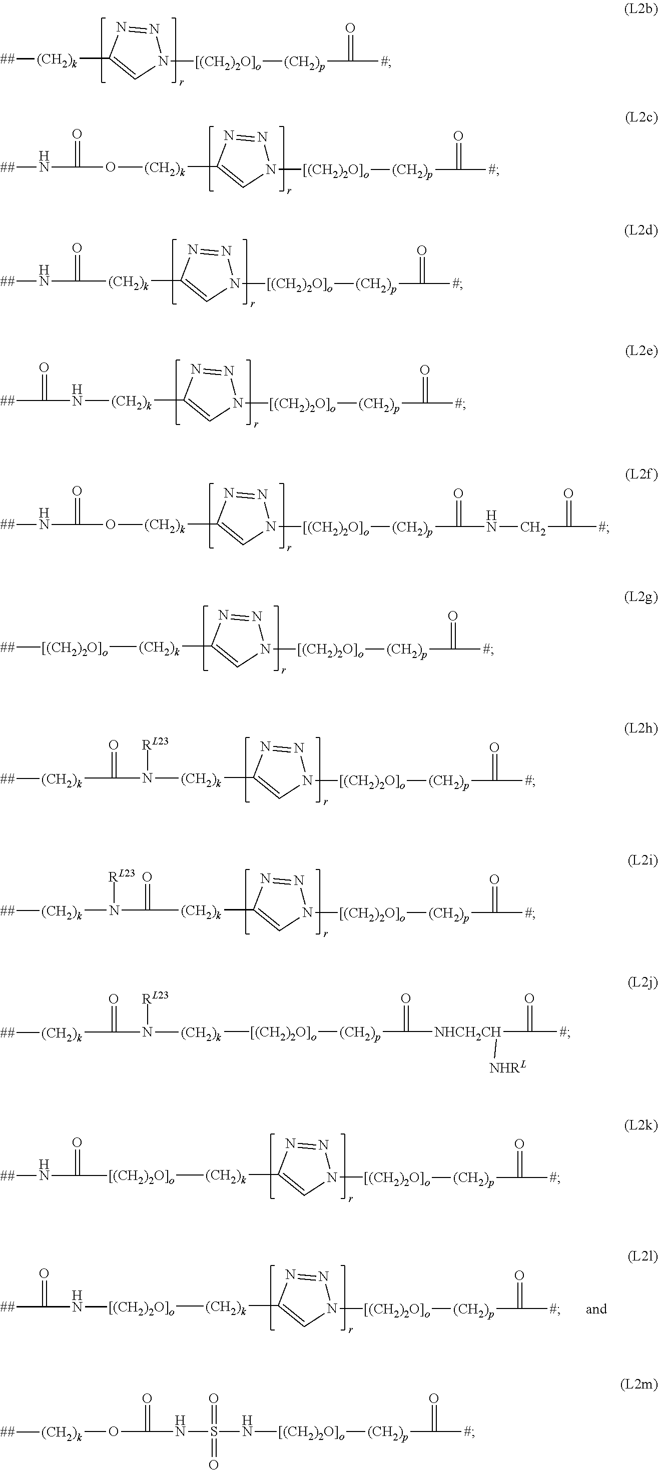

wherein
k, in each occurrence, is independently an integer from 0 to 4;
r, in each occurrence, is independently 0 or 1;
o, in each occurrence, is independently an integer from 0 to 10;
p, in each occurrence, is independently an integer from 0 to 4;
R^{L23} is hydrogen or C_{1 6}alkyl;
R^{L} is hydrogen or —C(O)—RH;
R^{H} is a hydrophilic group; and
the # of L² or L³ indicates the point of direct attachment to E¹ or E², respectively, and the ## of L² or L³ indicates the point of direct attachment to W;
provided that when W is N, L² and L³ are not (L2c), (L2d), (L2f), or (L2k).

The definitions of the remaining variables are provided in the eighteenth embodiment or any embodiments described therein.

In a twentieth embodiment, the present disclosure provides an antibody-drug conjugate of the nineteenth embodiment, wherein L² and L³ are each independently a connecting spacer selected from a group consisting of

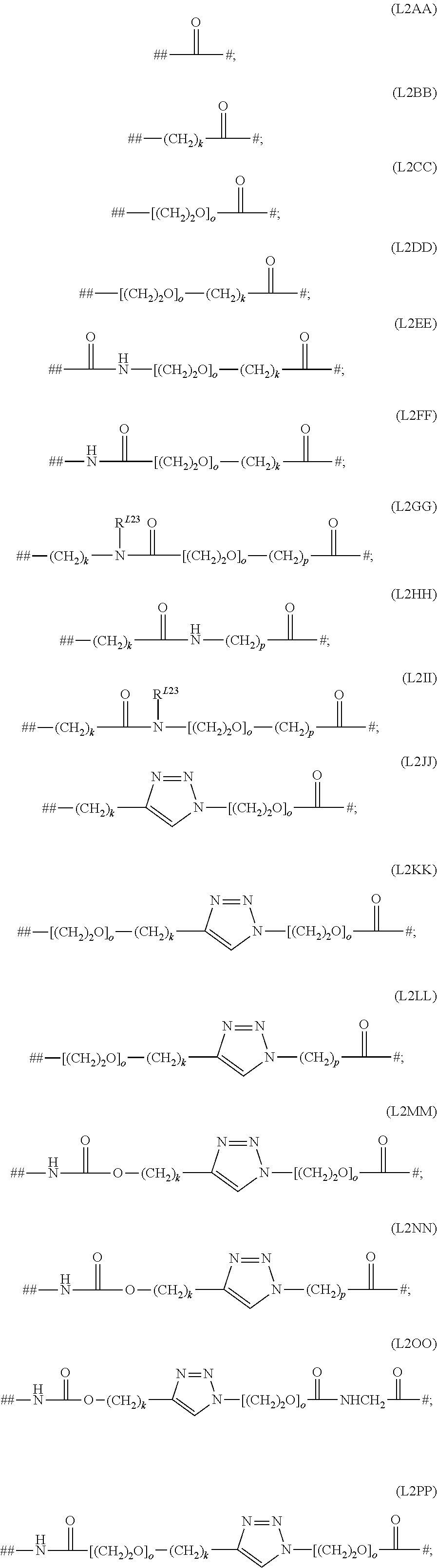

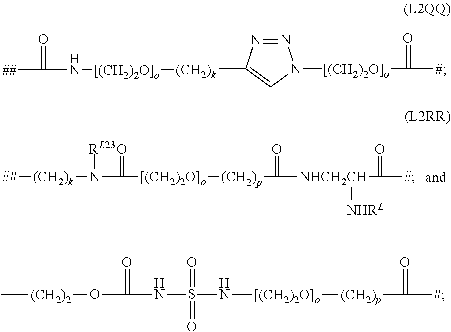

wherein
k, in each occurrence, is independently an integer from 1 to 3;
o, in each occurrence, is independently an integer from 1 to 9;
p, in each occurrence, is independently an integer from 1 to 3;
R^{L23} is hydrogen or C₁₋₃alkyl;
R^{L} is hydrogen or —C(O)—R^{H};
R^{H} is a hydrophilic group; and
the # of L² or L³ indicates the point of direct attachment to E¹ or E², respectively, and the ##of L² or L³ indicates the point of direct attachment to W;
provided that when W is N, L² and L³ are not (L2FF), (L2MM), (L2NN), (L200), or (L2PP).

The definitions of the remaining variables are provided in the nineteenth embodiment or any embodiments described therein.

In a twenty-first embodiment, the present disclosure provides an antibody-drug conjugate of the fifth through the twentieth embodiments, wherein:
L² and L³, independently, are a connecting spacer selected from a group consisting of

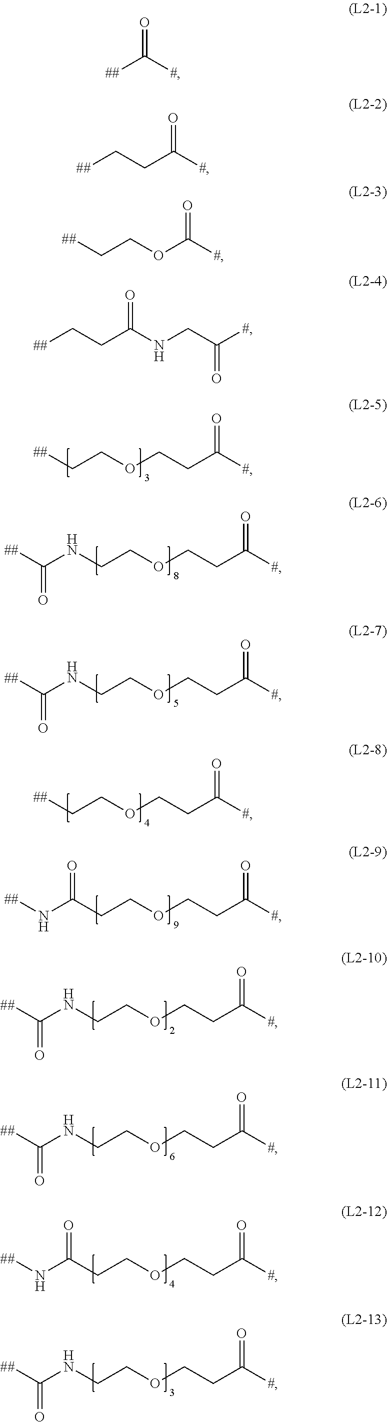

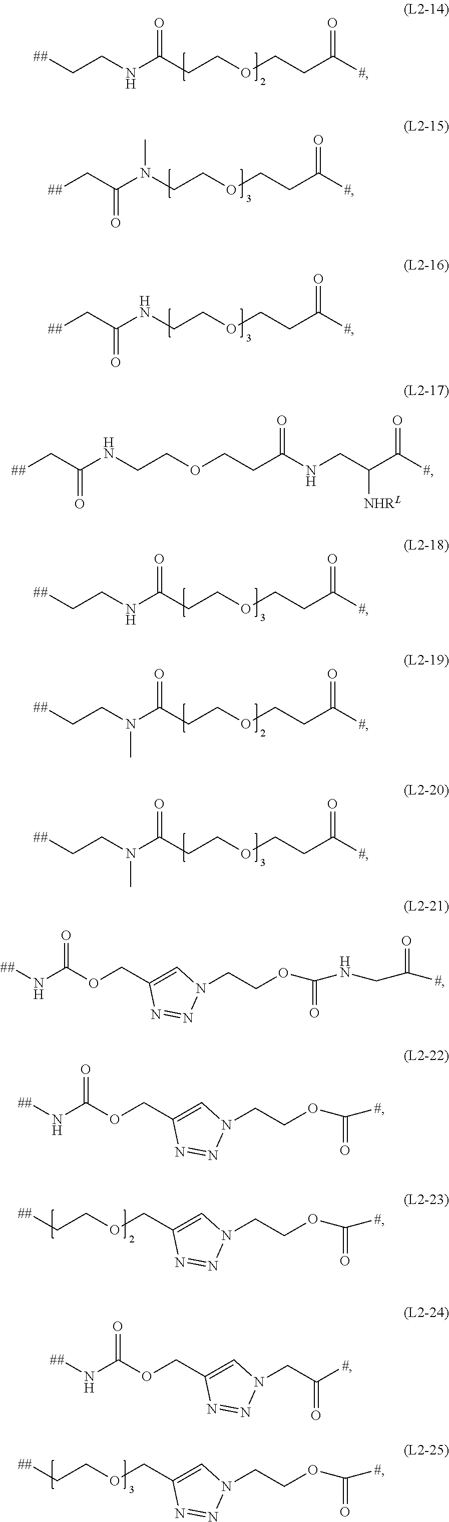

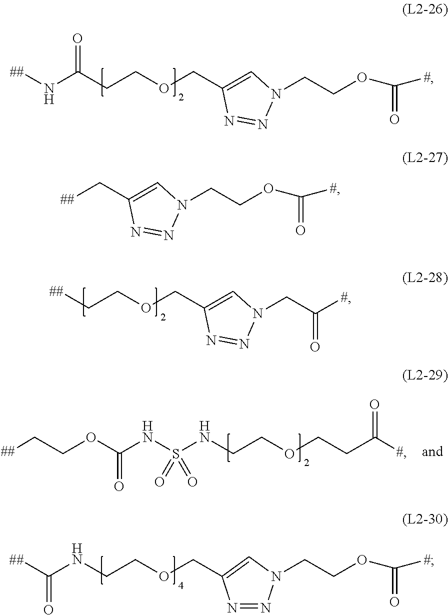

wherein the # of L² or L³ indicates the point of direct attachment to E¹ or E², respectively, the ## of L² or L³ indicates the point of direct attachment to W; R^{L} is hydrogen or —C(O)—R^{H}; and R^{H} is

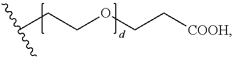

and d is an integer from 20 to 30 (e.g., 20, 21, 22, 23, 24, 25, 26, 27, 28, 29 or 30).

The definitions of the remaining variables are provided in the fifth through the twentieth embodiments or any embodiments described therein.

In a twenty-second embodiment, the present disclosure provides an antibody-drug conjugate of the twenty-first embodiment, wherein d is 25. The definitions of the remaining variables are provided in the twenty-first embodiments or any embodiments described therein.

In a twenty-third embodiment, the present disclosure provides an antibody-drug conjugate of any one of the seventh through the twenty-second embodiments, wherein the peptide group comprises 1 to 4, 1 to 3, or 1 to 2 amino acid residues. The definitions of the remaining variables are provided in the seventh through the twenty-second embodiments or any embodiments described therein.

In a twenty-fourth embodiment, the present disclosure provides an antibody-drug conjugate of the twenty-third embodiment, wherein the amino acid residues are selected from glycine (Gly), L-valine (Val), L-citrulline (Cit), L-cysteic acid (sulfo-Ala), L-lysine (Lys), L-isoleucine (Ile), L-phenylalanine (Phe), L-methionine (Met), L-asparagine (Asn), L-proline (Pro), L-alanine (Ala), L-leucine (Leu), L-tryptophan (Trp), L-tyrosine (Tyr) and β-alanine (β-Ala). The definitions of the remaining variables are provided in the twenty-third embodiment or any embodiments described therein.

In a twenty-fifth embodiment, the present disclosure provides an antibody-drug conjugate of any one of the first through the twenty-third embodiments, wherein the peptide group comprises Val-Cit, Phe-Lys, Val-Ala, Val-Lys, Leu-Cit, Cit-(j3-Ala), Gly-Gly-Gly, Gly-Gly-Phe-Gly, and/or sulfo-Ala-Val-Ala. The definitions of the remaining variables are provided in the first through the twenty-third embodiments or any embodiments described therein.

In a twenty-sixth embodiment, the present disclosure provides an antibody-drug conjugate of any one of the twenty-third through the twenty-fifth embodiments, wherein the peptide group represented by E¹ or E² is an enzyme cleavage element. The definitions of the remaining variables are provided in the twenty-third through the twenty-fifth embodiments or any embodiments described therein.

In a twenty-seventh embodiment, the present disclosure provides an antibody-drug conjugate of any one of the twenty-third through the twenty-fifth embodiments, or pharmaceutically acceptable salt thereof, wherein the peptide group represented by E¹ or E² is a hydrophilic moiety. The definitions of the remaining variables are provided in the twenty-third through the twenty-fifth embodiments or any embodiments described therein.

In a twenty-eighth embodiment, the present disclosure provides an antibody-drug conjugate of the twenty-sixth embodiment, or pharmaceutically acceptable salt thereof, wherein E¹ or E², independently, is an enzyme cleavage element selected from a group consisting of

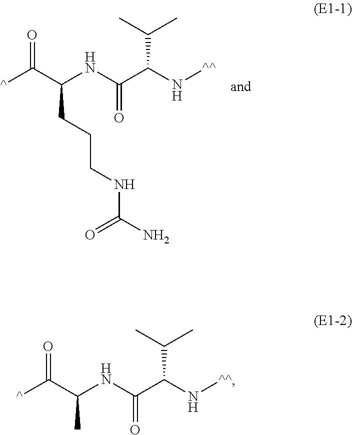

wherein \hat{ } of E¹ or E² indicates the point of direct attachment to V¹ or V² in Formula (B) or direct attachment to the —NH— group in Formula (C) and (D); and \hat{ }\hat{ } of E¹ or E² indicates the point of direct attachment to L² or L³, respectively. The definitions of the remaining variables are provided in the twenty-sixth embodiment or any embodiments described therein.

In a twenty-ninth embodiment, the present disclosure provides an antibody-drug conjugate of the twenty-seventh embodiment, or pharmaceutically acceptable salt thereof, wherein E¹ or E², independently, is a hydrophilic moiety represented by

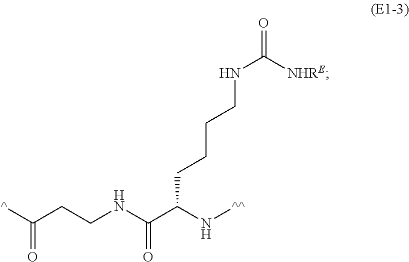

wherein R^{E} is a hydrophilic group R^{H}. The definitions of the remaining variables are provided in the twenty-seventh embodiment or any embodiments described therein.

In a thirtieth embodiment, the present disclosure provides an antibody-drug conjugate of the twenty-ninth embodiment, or pharmaceutically acceptable salt thereof, wherein each hydrophilic

group R^{H} in E¹ or E² is independently

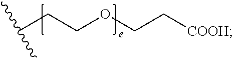

wherein e is an integer between 20 and 30 (e.g., 20, 21, 22, 23, 24, 25, 26, 27, 28, 29 or 30). The definitions of the remaining variables are provided in the twenty-ninth embodiment or any embodiments described therein.

In a thirty-first embodiment, the present disclosure provides an antibody-drug conjugate of the thirtieth embodiment, wherein e is 24. The definitions of the remaining variables are provided in the thirtieth embodiment or any embodiments described therein.

In a thirty-second embodiment, the present disclosure provides an antibody-drug conjugate of any one of the seventh through the thirty-first embodiments, or pharmaceutically acceptable salt thereof, wherein A¹ and A² independently are a bond, —OC(═O)—*, or

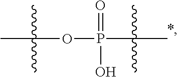 [SMILES: *OP(*)(=O)O]

wherein * indicates the point of attachment to D¹ or D². The definitions of the remaining variables are provided in the seventh through the thirty-first embodiments or any embodiments described therein. In some embodiments, A¹ and A² independently are a bond or

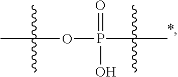 [SMILES: *OP(*)(=O)O]

wherein * indicates the point of attachment to D¹ or D². In some embodiments, A¹ and A² independently are a bond or —OC(═O)—*, wherein * indicates the point of attachment to D¹ or D². In some embodiments, A¹ and A² are as defined: (i) A¹ and A² are —OC(═O)—*; (ii) A¹ and A² are

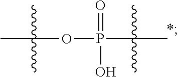 [SMILES: *OP(*)(=O)O]

(iii) A¹ is —OC(═O)—* and A² is a bond; (iv) A¹ is —OC(═O)—* and A² is

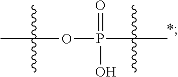 [SMILES: *OP(*)(=O)O]

(v) A¹ is a bond and A² is

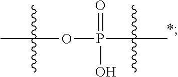 [SMILES: *OP(*)(=O)O]

or (vi) A¹ is a bond and A² is —OC(═O)—*, wherein * indicates the point of attachment to D¹ or D².

In a thirty-third embodiment, the present disclosure provides an antibody-drug conjugate of any one of the seventh through the thirty-second embodiments, wherein A¹ and A² are a bond. The definitions of the remaining variables are provided in the seventh through the thirty-second embodiments or any embodiments described therein.

In a thirty-fourth embodiment, the present disclosure provides an antibody-drug conjugate of any one of the seventh through the thirty-third embodiments, wherein
i) L⁴ and^{L} are each independently a spacer moiety having the structure

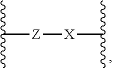

wherein:
Z is —O—, —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{L45}—, —C(═O)NH—, —CH₂NR^{L45} C(═O)—, —CH₂NR^{L45}C(═O)NH—, —CH₂NR ^{L45}C(═O)NR^{L45} —NHC(═O)—, —NHC(═O)O—, —NHC(═O)NH—, —OC(═O)NH—, —S(O)₂NH—, —NHS(O)₂—, —C(═O)—, —C(═O)O- or —NH—, wherein each R^{L45} is independently selected from H, C₁-C₆alkyl, and C₃-C₅ cycloalkyl; and
X is a bond, triazolyl, or —CH₂-triazolyl-,
wherein X is connected to R² or R³; or
(ii) L⁴ and L⁵, independently, are a spacer moiety having the structure

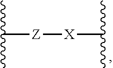

wherein:
Z is —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{b}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O- or —NH—, wherein each R^{L45} is independently selected from H, C₁-C₆alkyl, and C₃-C₅ cycloalkyl; and
X is —CH₂-triazolyl-C₁₋₄ alkylene-OC(O)NHS(O)₂NH—, —C₄₆ cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂-triazolyl-C₁₋₄ alkylene-OC(O)NHS(O)₂NH—(CH₂CH₂O)ₙ—, —C₄₋₆cycloalkylene-OC(O)NHS(O)₂NH—(CH₂CH₂O)ₙ—,wherein each n independently is 1, 2, or 3,
wherein X is connected to R² or R³.

The definitions of the remaining variables are provided in the seventh through the thirty-third embodiments or any embodiments described therein.

In a thirty-fifth embodiment, the present disclosure provides an antibody-drug conjugate of the thirty-fourth embodiment, or pharmaceutically acceptable salt thereof, wherein Z is —O—, —CH₂NR^{L45} C(═O)—, —CH₂NR^{L45}C(═O)NH— or —CH₂O—; X is a bond, triazolyl, or —CH₂-triazolyl-; and RM, in each occurrence, is independently H or C₁_3alkyl. The definitions of the remaining variables are provided in the thirty-fourth embodiment or any embodiments described therein.

In a thirty-sixth embodiment, the present disclosure provides an antibody-drug conjugate of any one of the seventh through the thirty-fifth embodiments, or pharmaceutically acceptable salt thereof, wherein L⁴ and L⁵are each independently a spacer moiety selected from a group consisting of

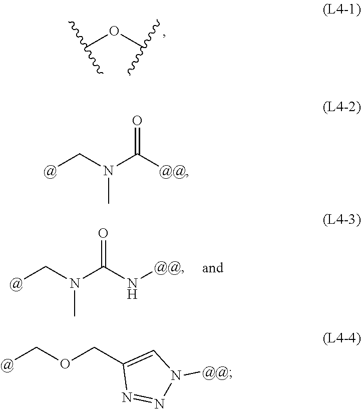

wherein the @ of L⁴or L⁵ indicates the point of direct attachment to the phenyl group, and the @@ of L⁴ or L⁵ indicates the point of direct attachment to R² or R³. The definitions of the remaining variables are provided in the seventh through the thirty-fifth embodiments or any embodiments described therein.

In a thirty-seventh embodiment, the present disclosure provides an antibody-drug conjugate of any one of the seventh through the thirty-sixth embodiments, wherein the hydrophilic groups represented by R² and R³ each independently comprises polyethylene glycol, polyalkylene glycol, a polyol, a polysarcosine, a sugar, an oligosaccharide, a polypeptide, C₂-C₆ alkyl substituted with 1 to 3

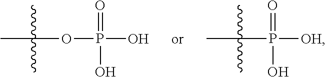 [SMILES: *OP(=O)(O)O ; *P(=O)(O)O]

or C₂-C₆alkyl substituted with 1 to 2 substituents independently selected from —OC(═O)NHS(O)₂NHCH₂CH₂OCH₃, —NHC(═O)C₁₋₄alkylene-P(O)(OCH₂CH₃)₂ and —COOH groups. The definitions of the remaining variables are provided in the seventh through the thirty-sixth embodiments or any embodiments described therein.

In a thirty-eighth embodiment, the present disclosure provides an antibody-drug conjugate of any one of the seventh through the thirty-seventh embodiments, wherein R² or R³ independently is

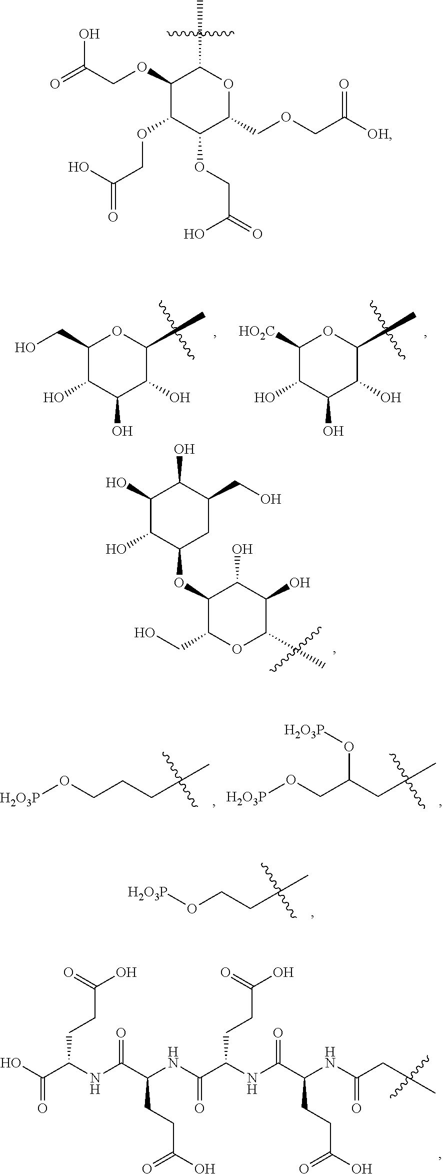

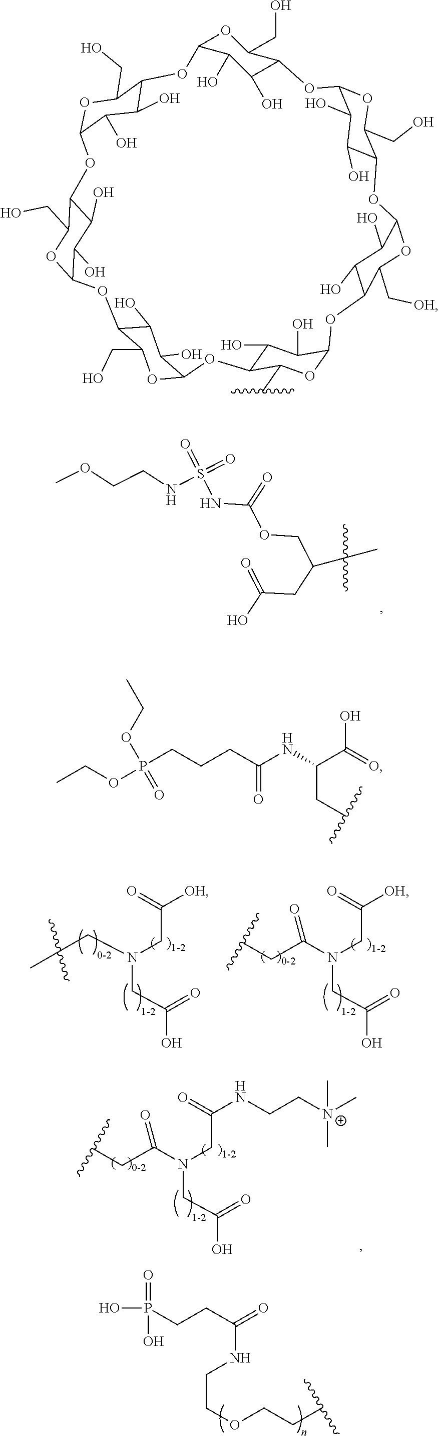

wherein n is an integer between 1 and 6,

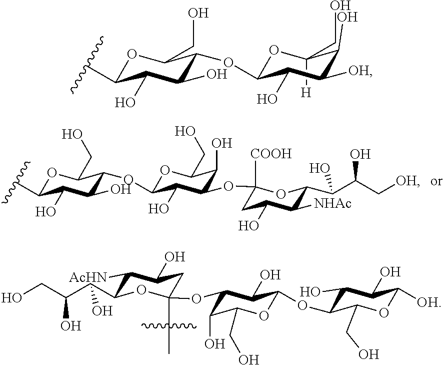

The definitions of the remaining variables are provided in the seventh through the thirty-seventh embodiments or any embodiments described therein.

In a thirty-ninth embodiment, the present disclosure provides an antibody-drug conjugate of any one of the seventh through the thirty-eighth embodiments, wherein the hydrophilic group represented by R² or R³ each independently comprises:

(i) a polysarcosine with the following moiety:

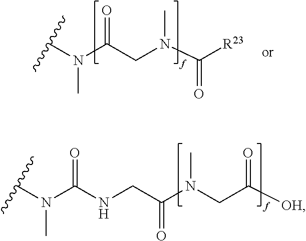

wherein
f is an integer between 3 and 25; and
R²³ is H, —CH₃ or —CH₂CH₂C(═O)OH; or

(ii) a polyethylene glycol of formula:

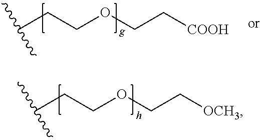

wherein
g and h are independently an integer between 2 and 30.

In some embodiments, the hydrophilic group represented by R² or R³ each independently comprises:
a polysarcosine with the following moiety:

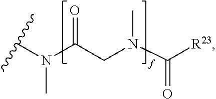

wherein
f is an integer between 3 and 25; and
R²³ is H, —CH₃ or —CH₂CH₂C(═O)OH.

The definitions of the remaining variables are provided in the seventh through the thirty-eighth embodiments or any embodiments described therein.

In a fortieth embodiment, the present disclosure provides an antibody-drug conjugate of any one of the seventh through the thirty-sixth embodiments, wherein the enzyme cleavage element represented by R² or R³ each independently comprises:

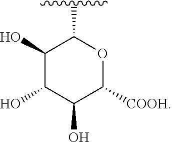 [SMILES: *[C@@H]1O[C@H](C(=O)O)[C@@H](O)[C@H](O)[C@H]1O]

The definitions of the remaining variables are provided in the seventh through the thirty-sixth embodiments or any embodiments described therein.

In a forty-first embodiment, the present disclosure provides an antibody-drug conjugate of any one of the seventh through the thirty-sixth embodiments, wherein R² or R³, independently, is COOH OCH₃ selected from a group consisting of

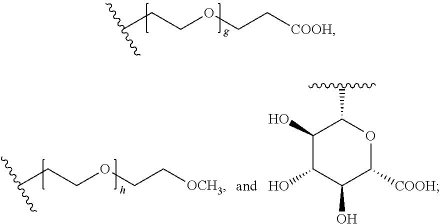

wherein g and h are independently an integer between 20 and 30.

The definitions of the remaining variables are provided in the seventh through the thirty-sixth embodiments or any embodiments described therein.

In a forty-second embodiment, the present disclosure provides an antibody-drug conjugate of the thirty-ninth through the forty-first embodiments, wherein
g is 23, 24, or 25; and
h is 23, 24, or 25.

The definitions of the remaining variables are provided in the thirty-ninth through the forty-first embodiments or any embodiments described therein.

In a forty-third embodiment, the present disclosure provides an antibody-drug conjugate of the seventh embodiment, wherein the dual linker is represented by the following formula:

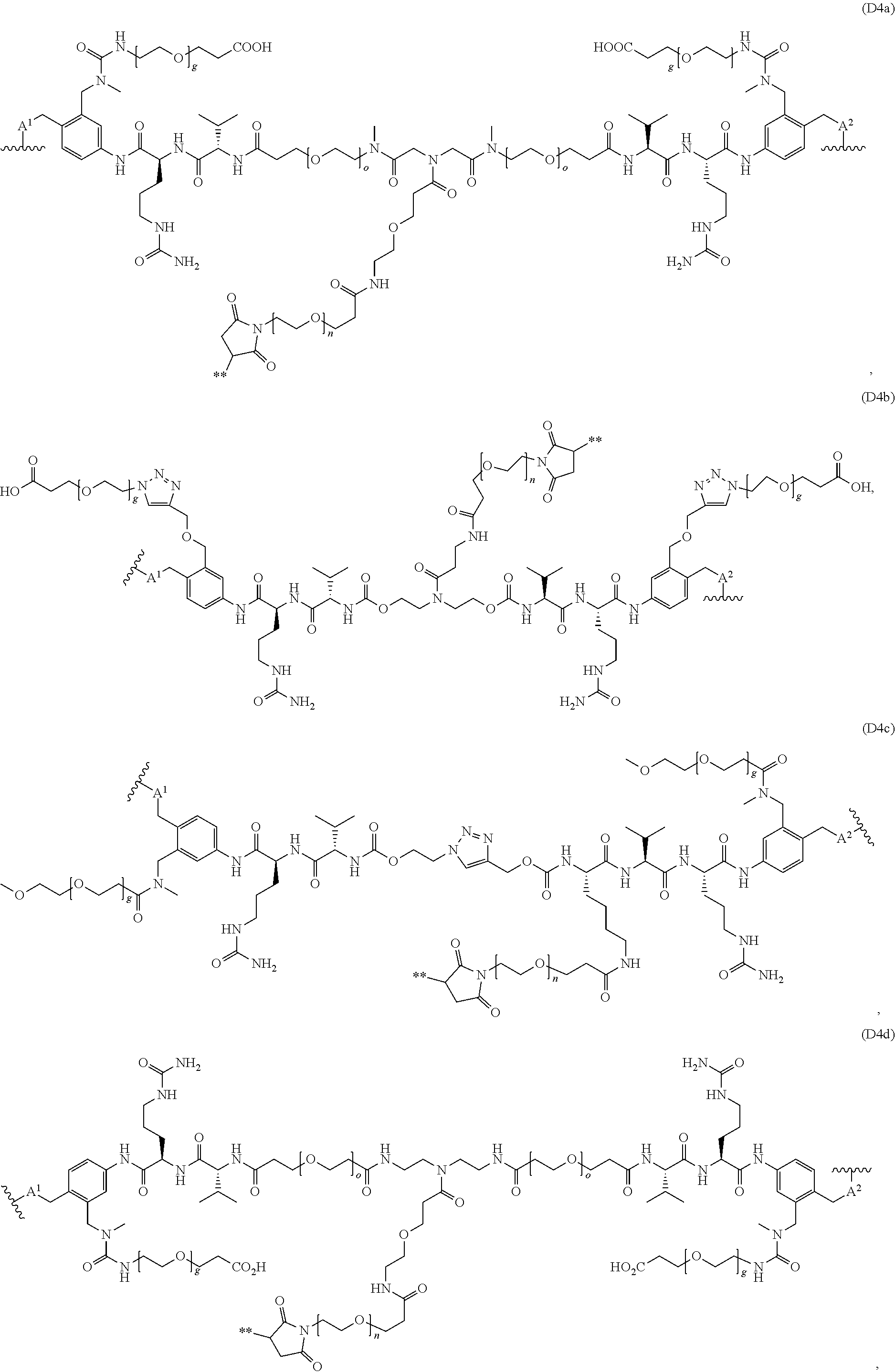

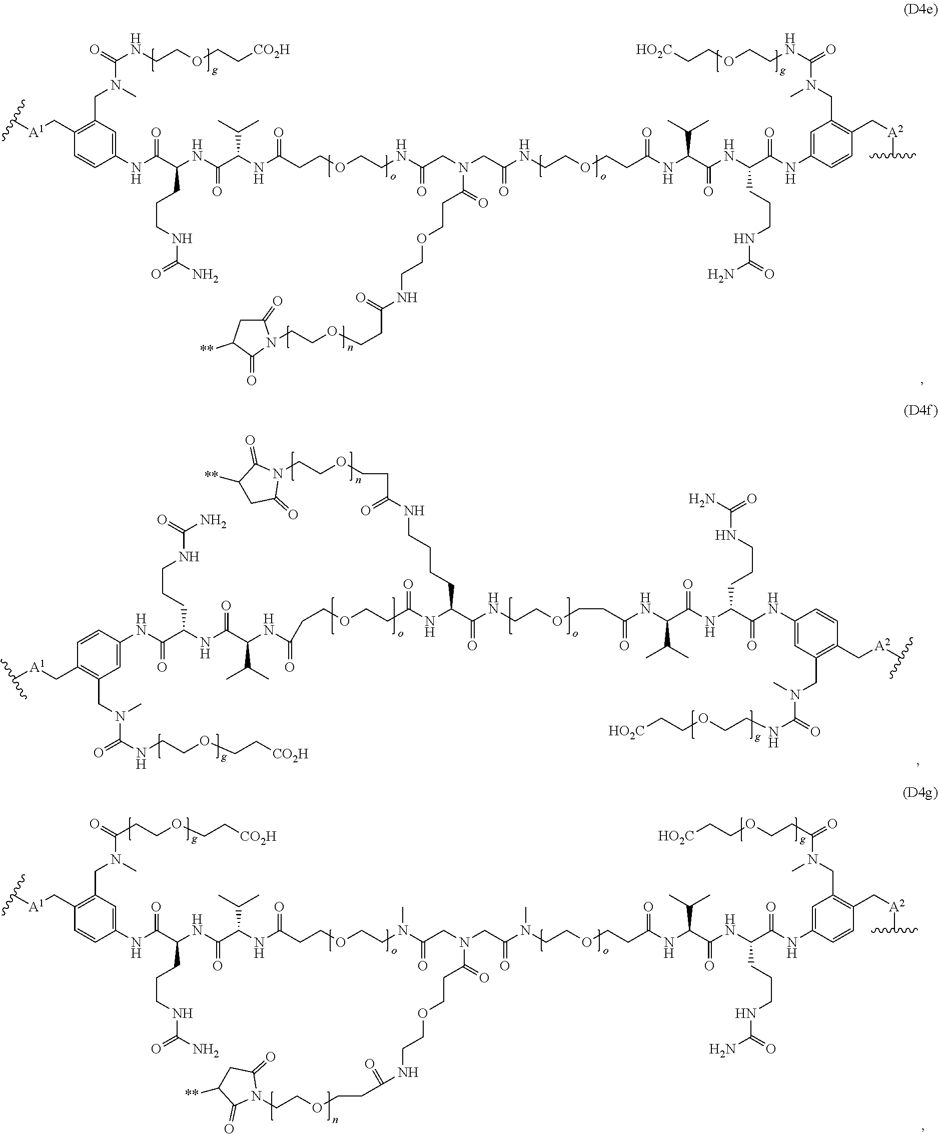

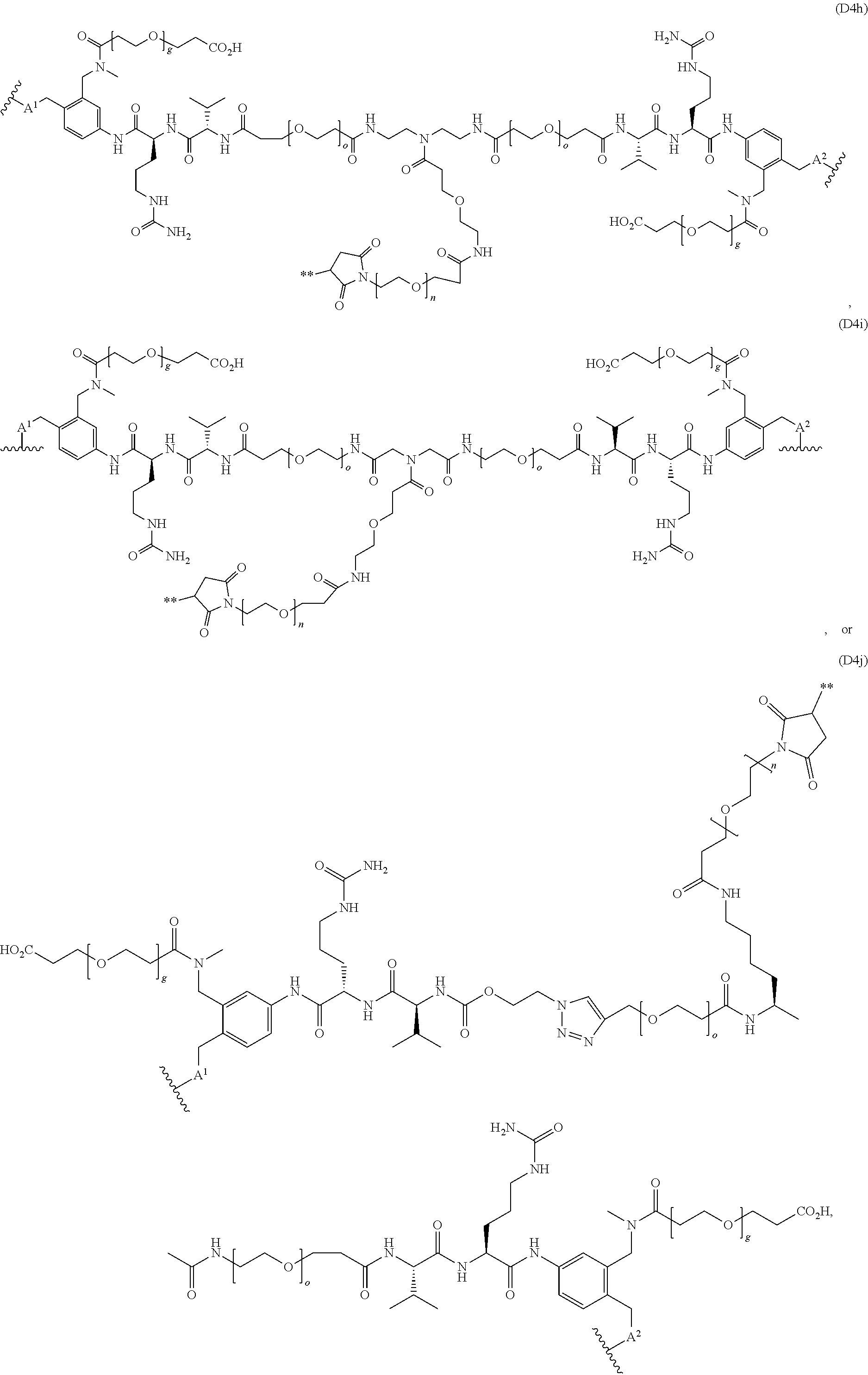

wherein:
A¹ and A² are each independent a bond, —O—C(═O)—*, or

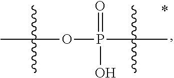 [SMILES: *OP(*)(=O)O]

wherein * in A¹ and A² indicates the point of attachment to D¹ or D²
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 2 and 5);
n is an integer between 1 and 12 (e.g., between 2 and 5);
** [symbol] indicates the point of attachment to the Ab; and
[symbol] indicates the point of direct attachment to D¹ or D².

The definitions of the remaining variables are provided in the seventh embodiment or any embodiments described therein. In some embodiments, A¹ and A² are each independent a bond or —O—C(═O)—*, wherein * in A¹ and A² indicates the point of attachment to D¹ or D². In some embodiments, A¹ and A² are both bond. In some embodiments, A¹ and A² are both —OC(═O)—*. In some embodiments, one of A¹ and A² is a bond and the other is OC(═O)—*.

In a forty-fourth embodiment, the present disclosure provides an antibody-drug conjugate of the seventh embodiment, wherein the dual linker is represented by Formula (D5):

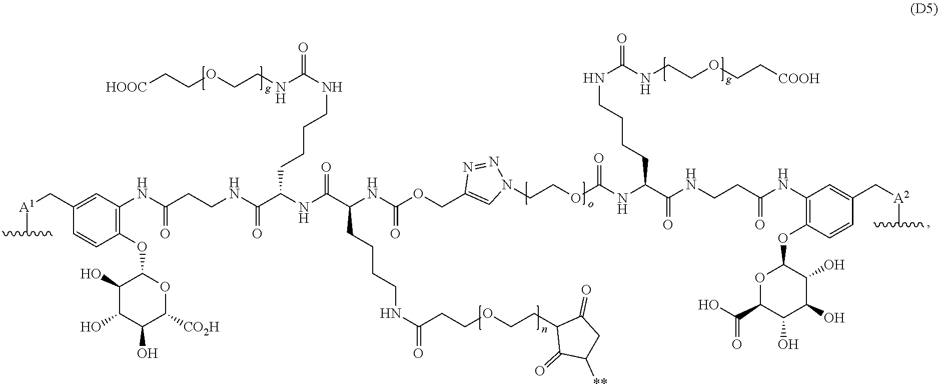

wherein:
A¹ and A² are each independent a bond, —O—C(═O)—* or

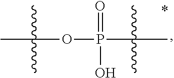 [SMILES: *OP(*)(=O)O]

wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 1 and 3);
n is an integer between 1 and 12 (e.g., between 5 and 10);
[symbol] indicates the point of attachment to the Ab; and
[symbol] indicates the point of direct attachment to D¹ or D². The definitions of the remaining variables are provided in the seventh embodiment or any embodiments described therein. In some embodiments, A¹ and A² are each independent a bond or —O—C(═O)—*, wherein * in A¹ and A² indicates the point of attachment to D¹ or D².

In a forty-fifth embodiment, the present disclosure provides an antibody-drug conjugate of the first embodiment, or pharmaceutically acceptable salt thereof, wherein the dual linker is represented by the following formula:

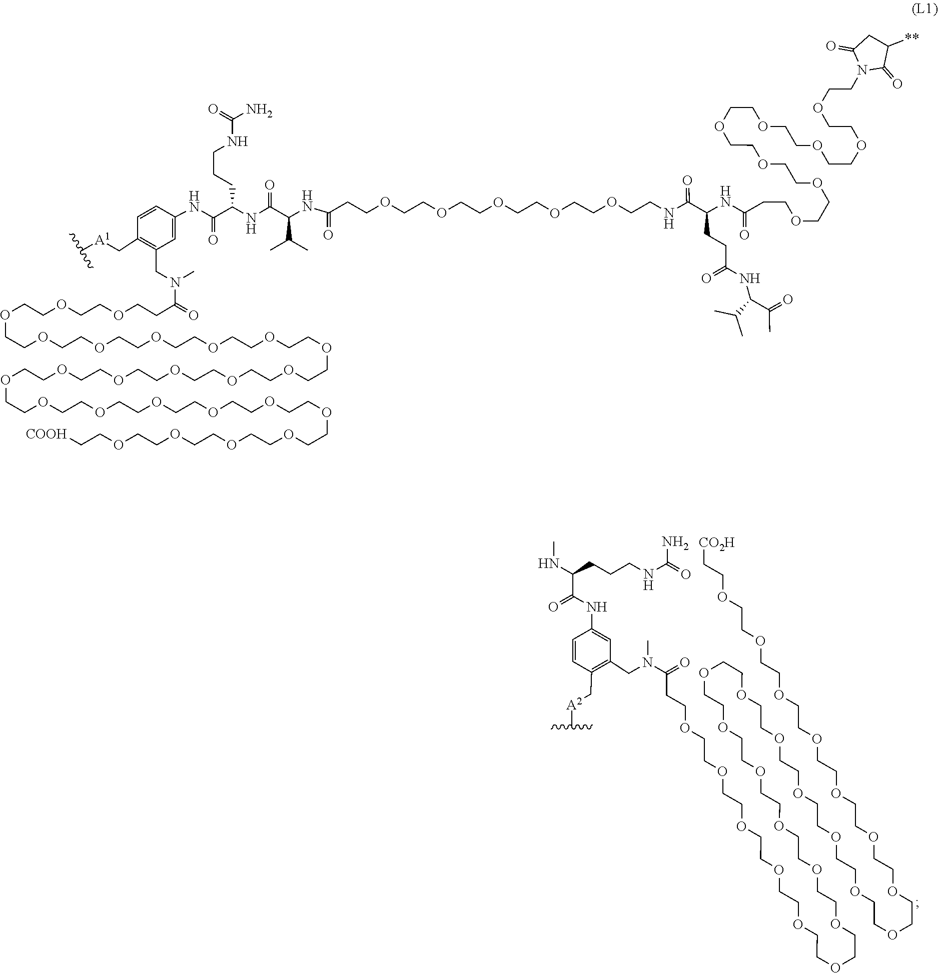

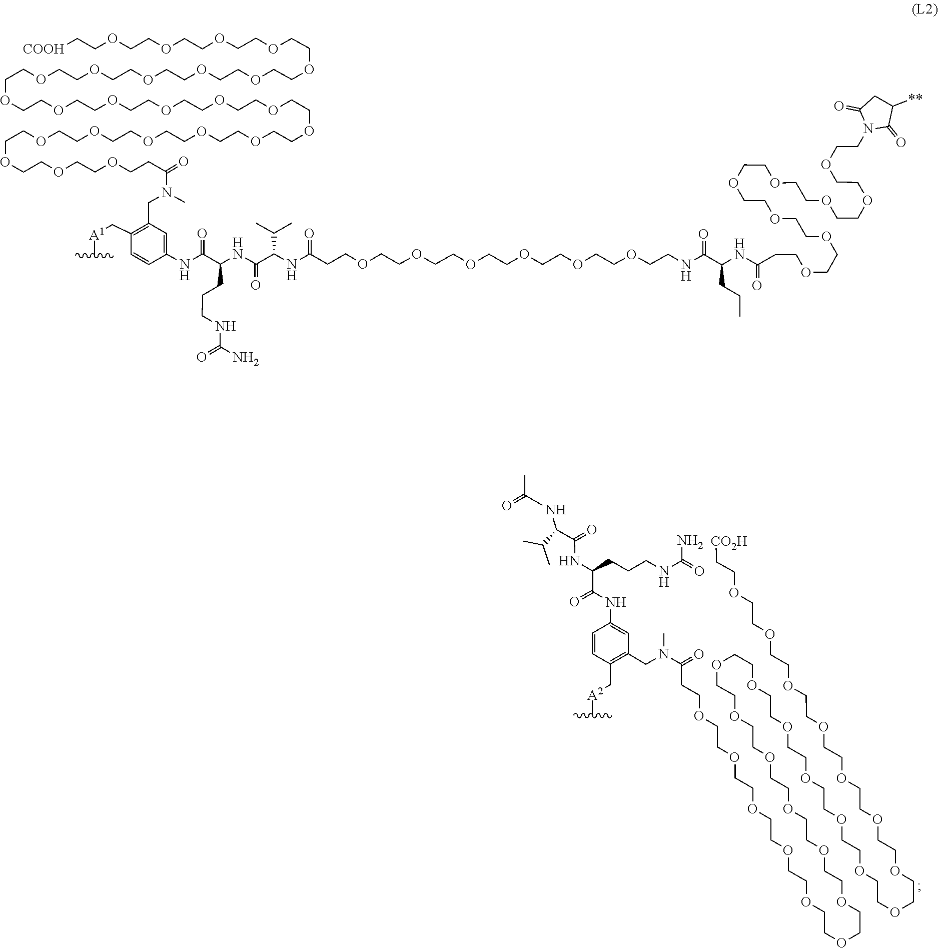

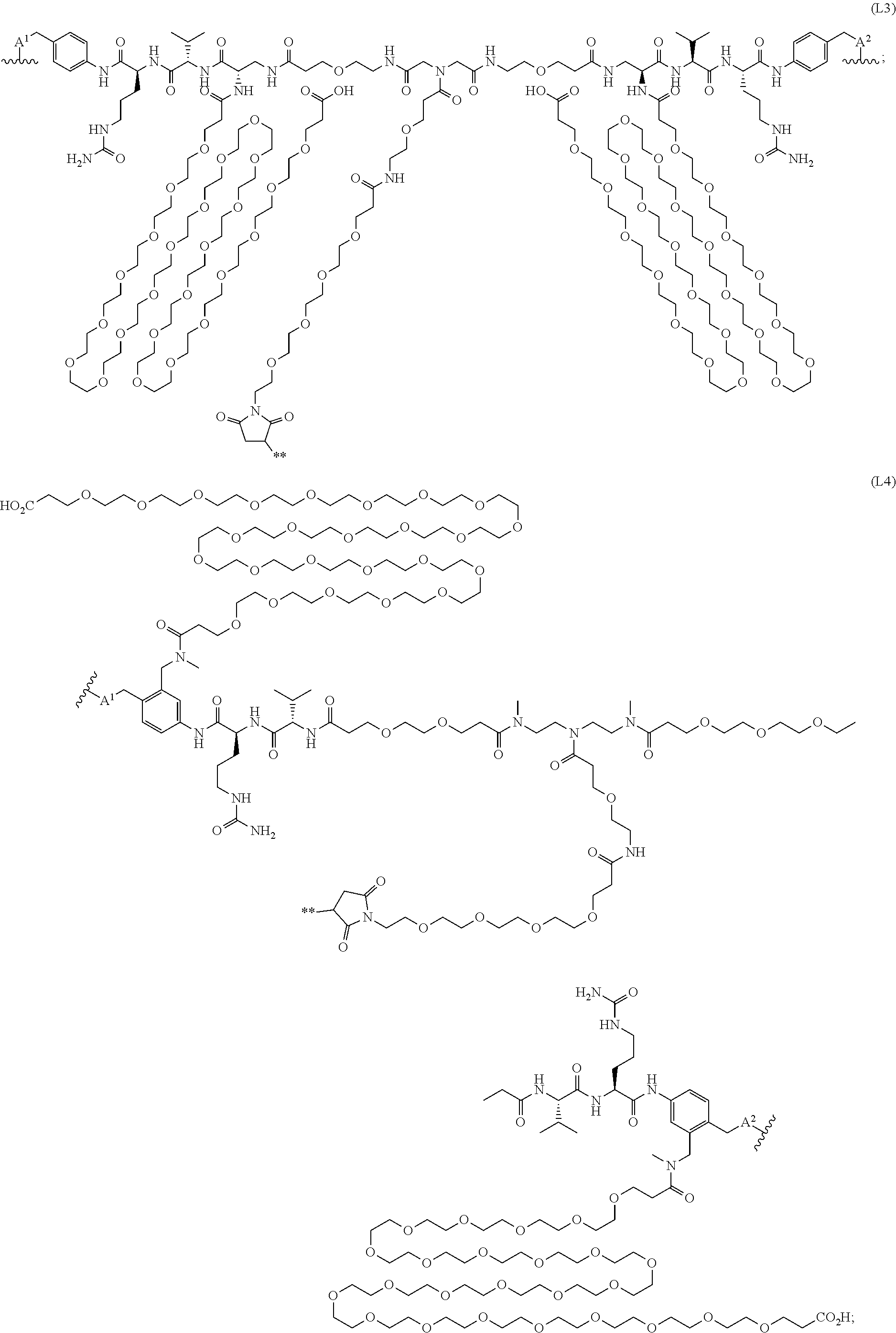

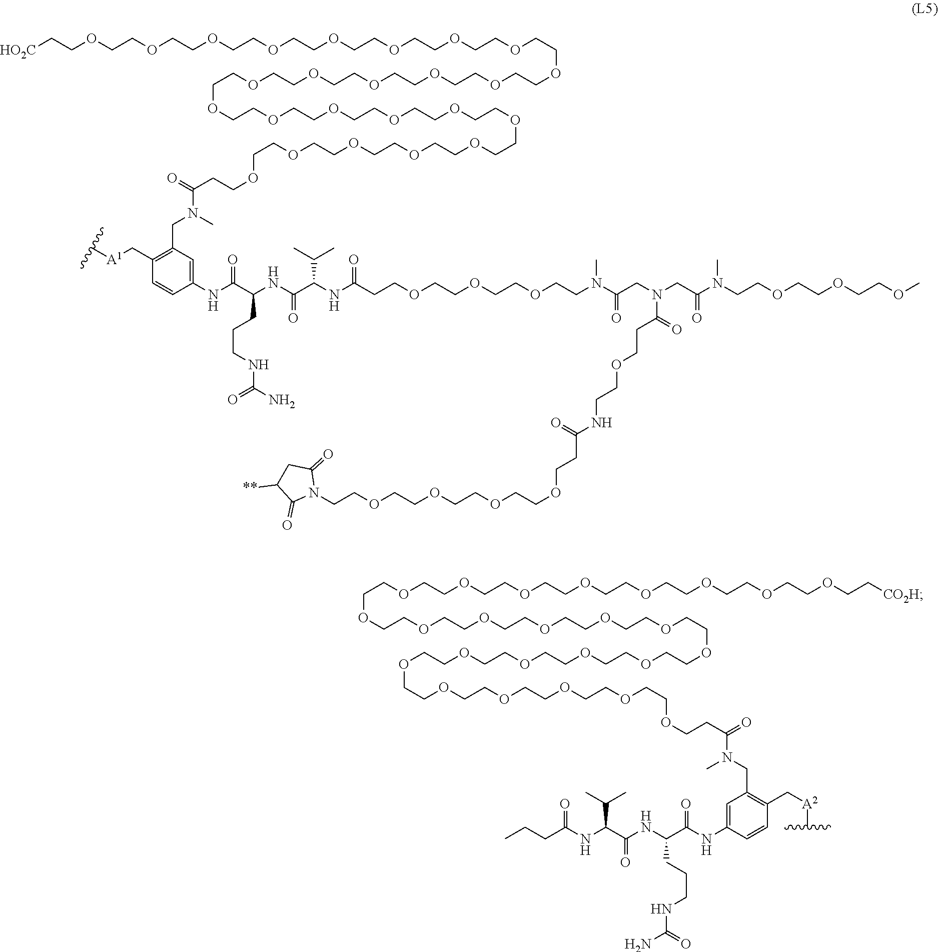

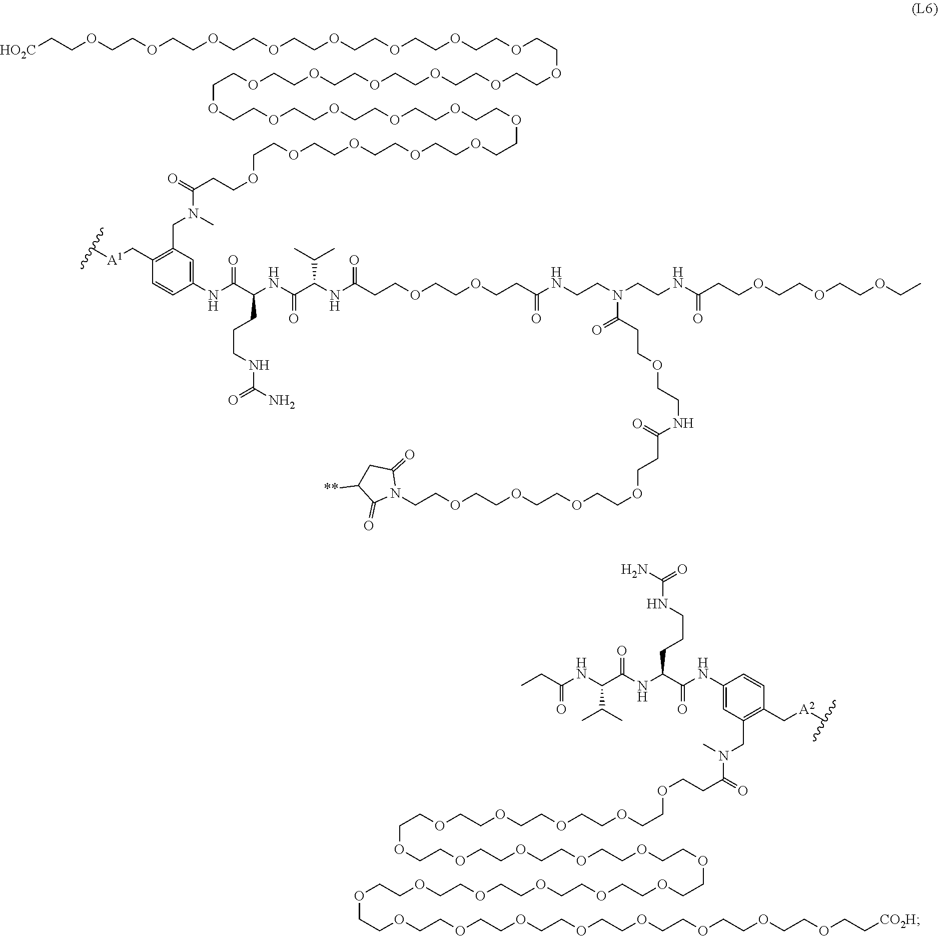

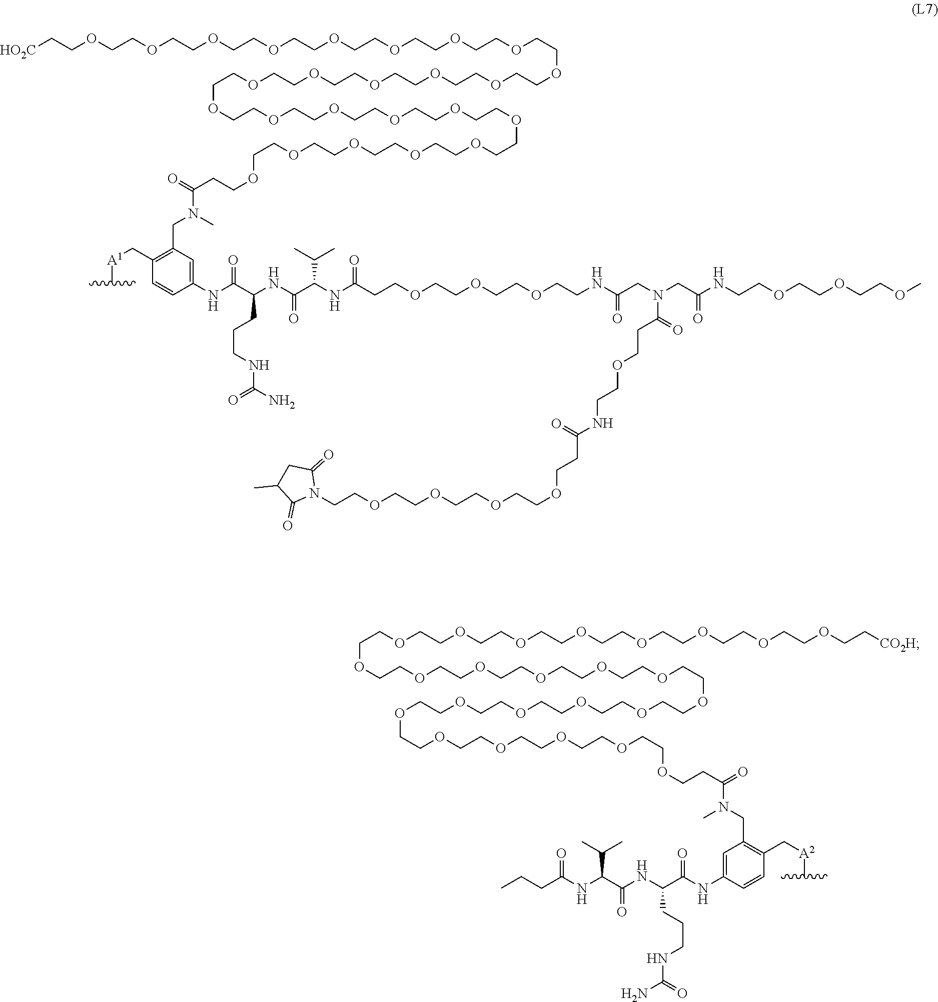

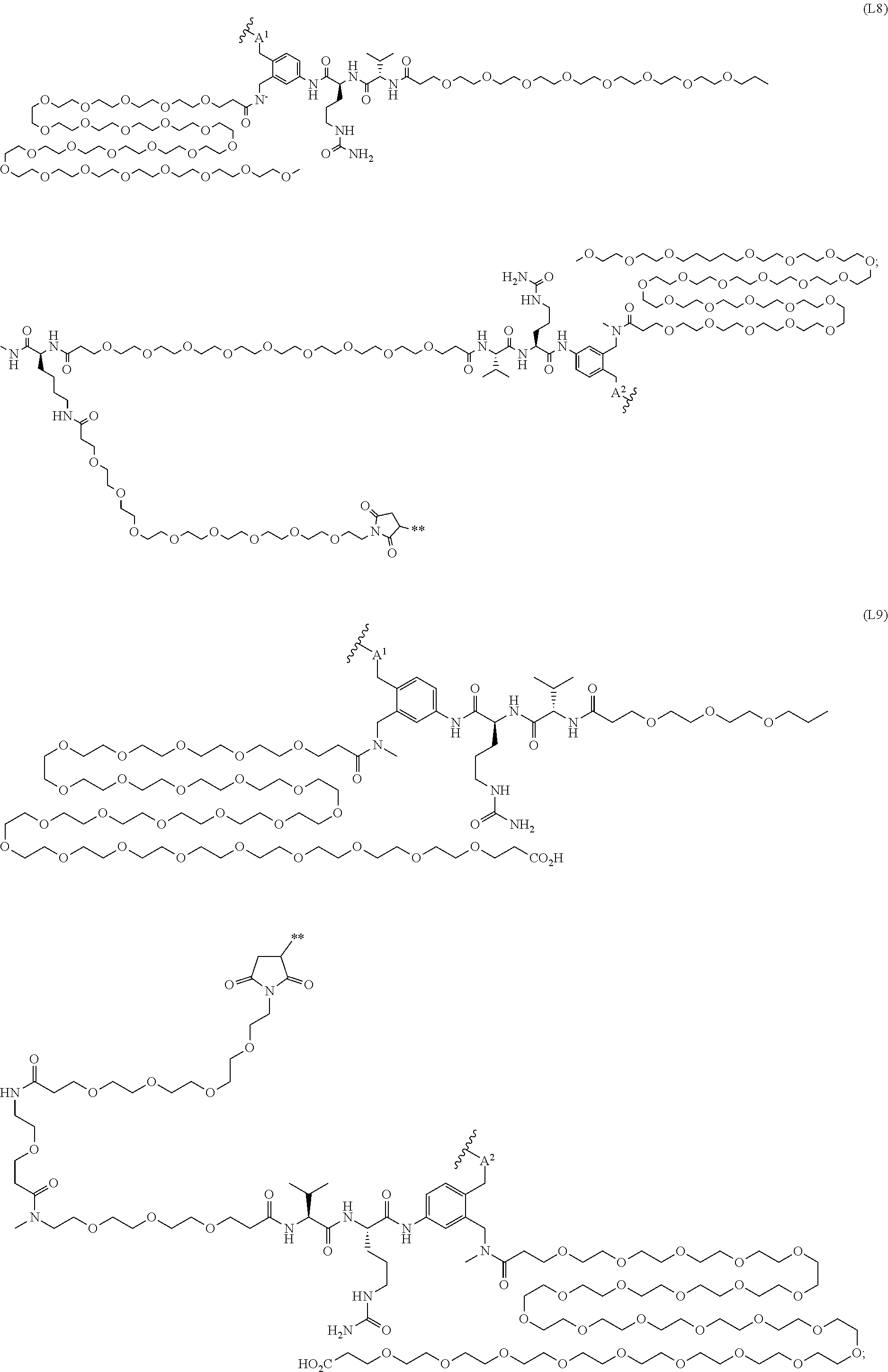

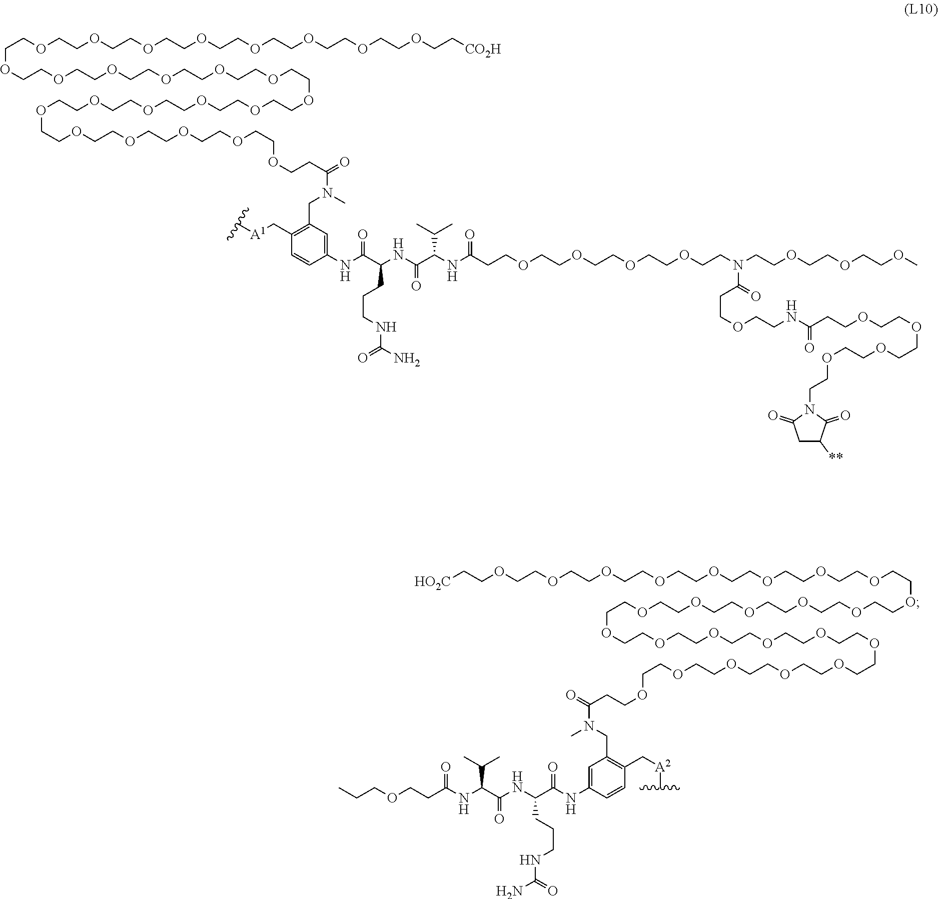

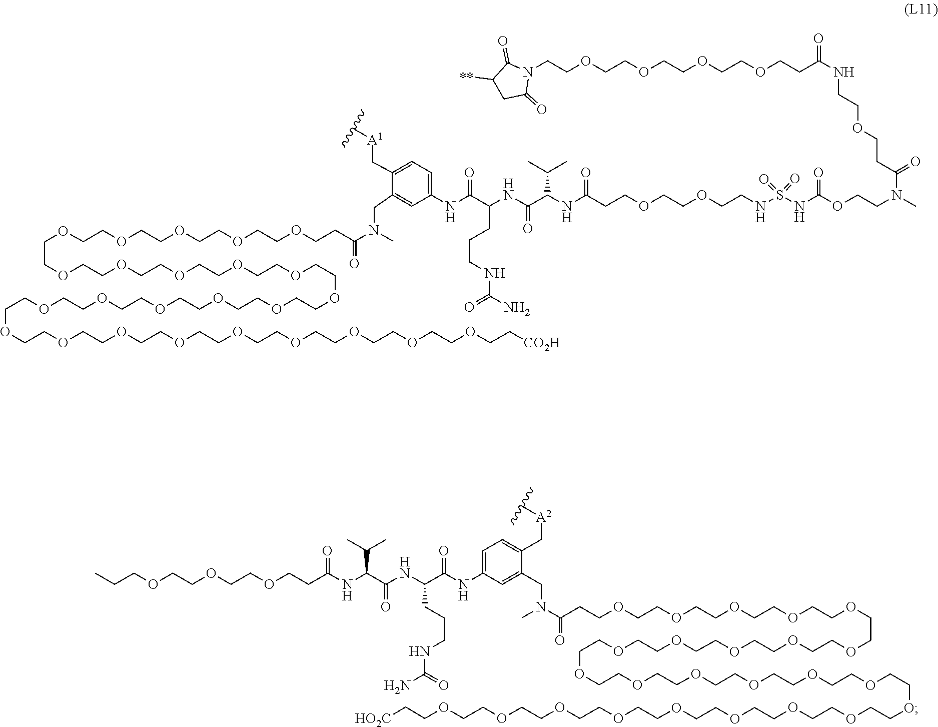

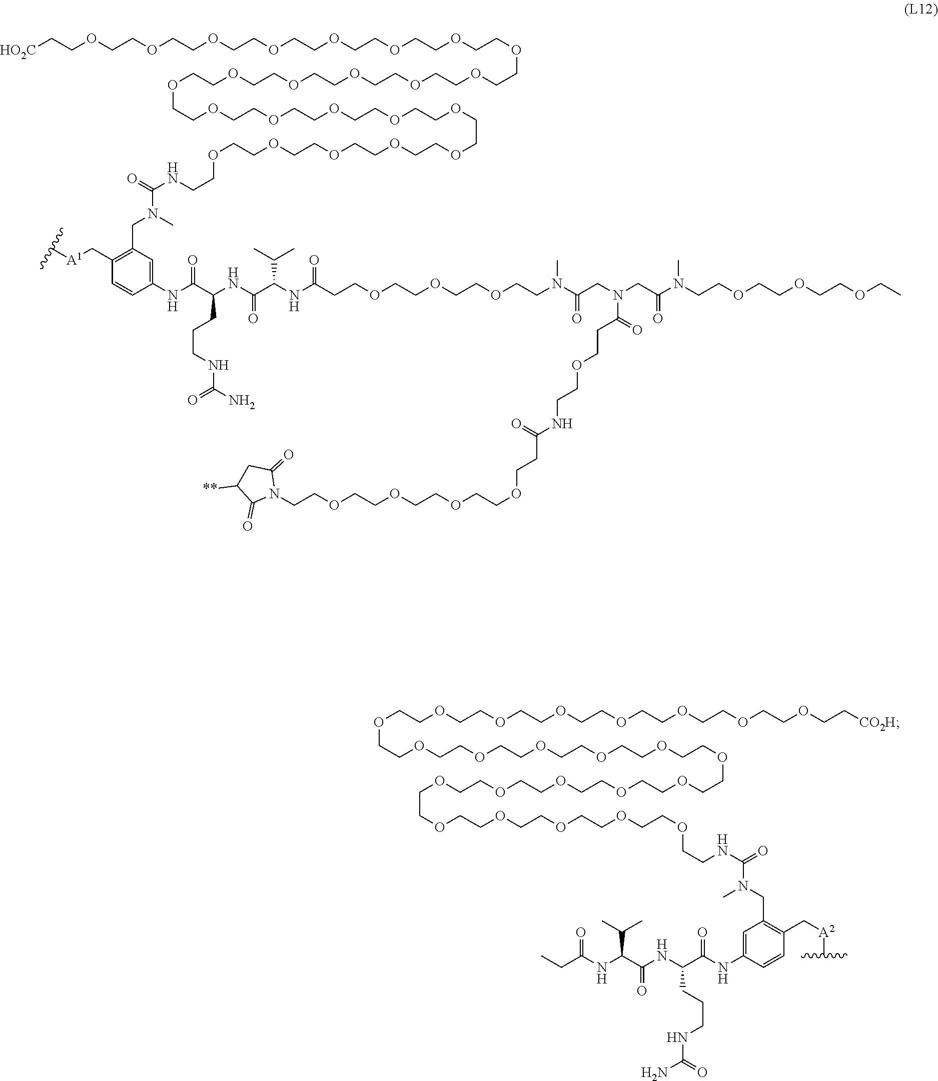

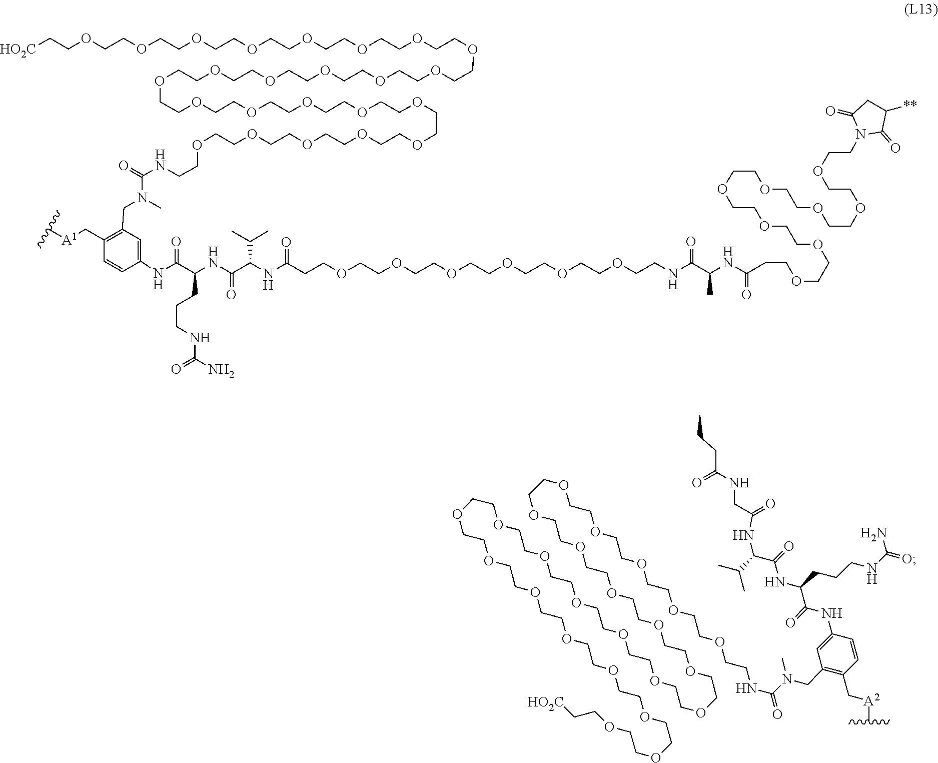

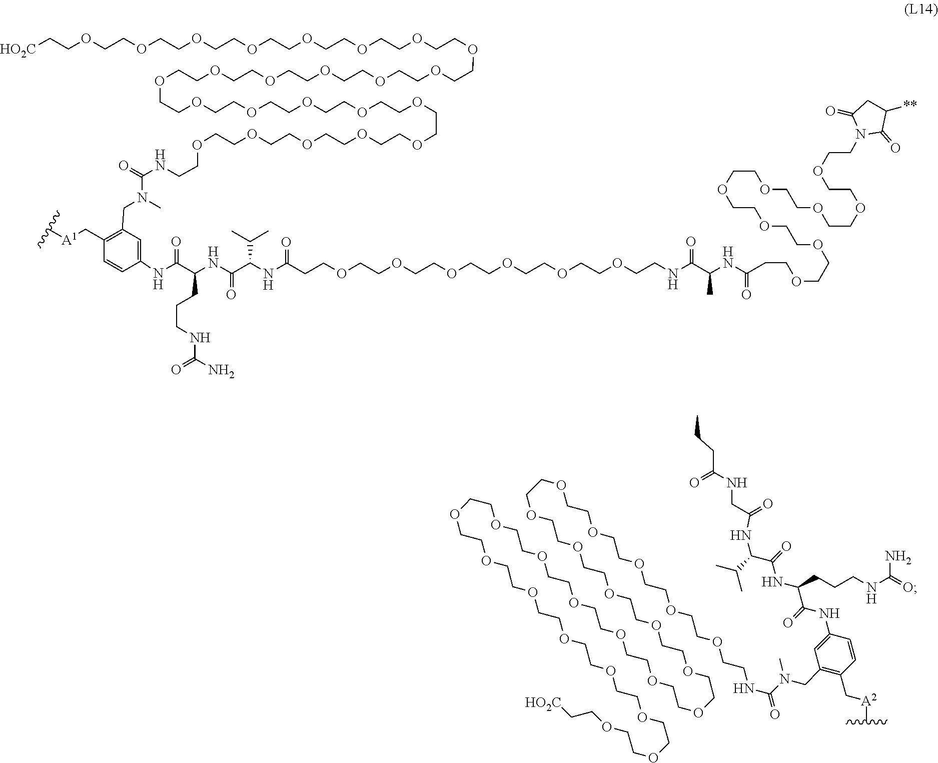

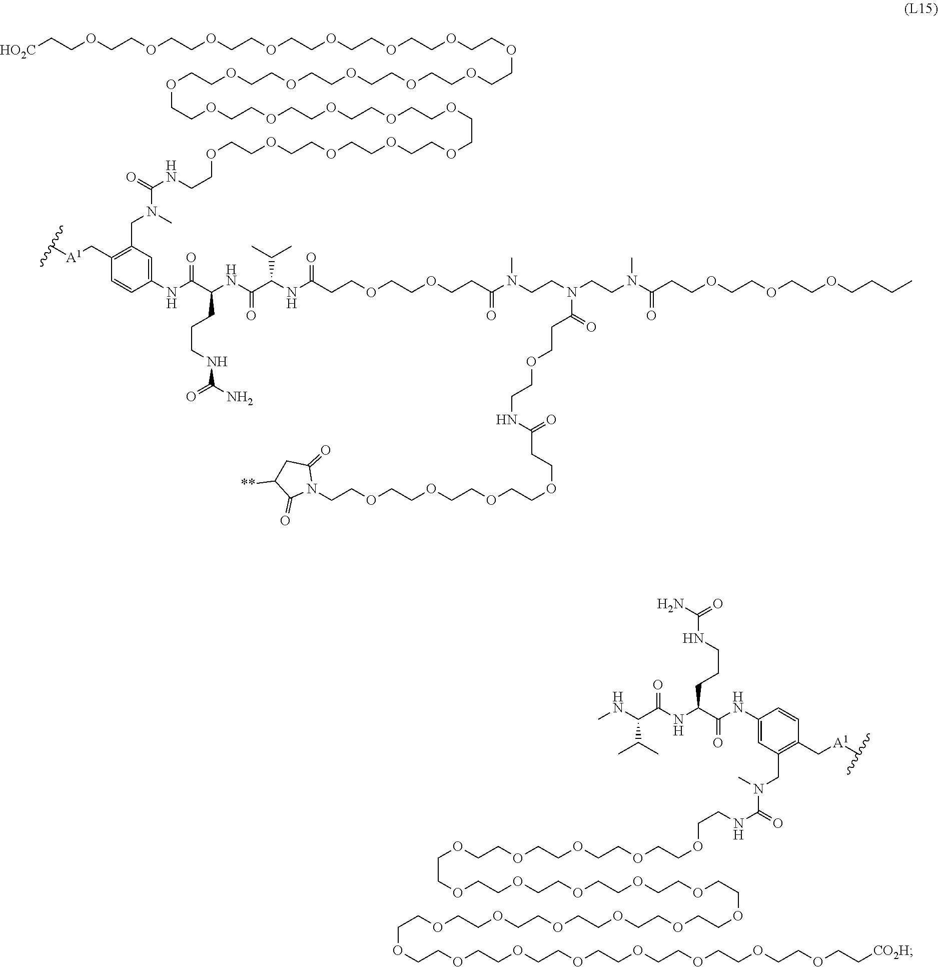

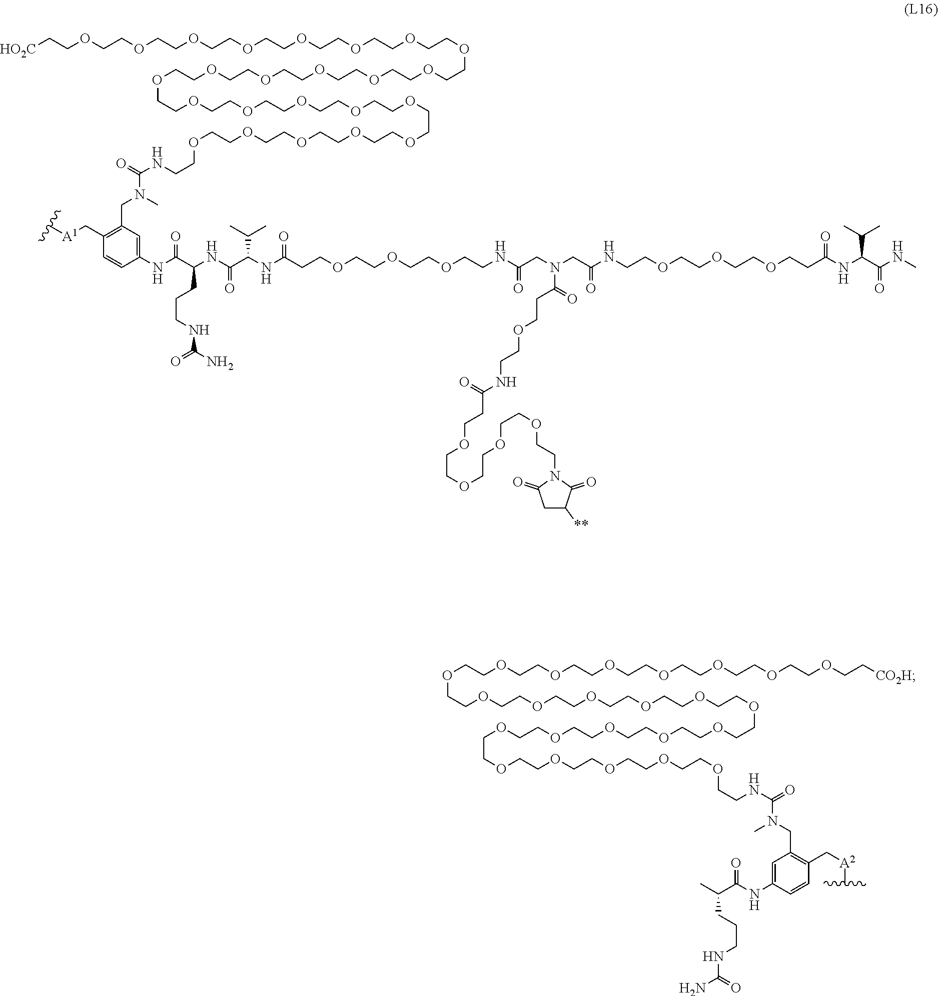

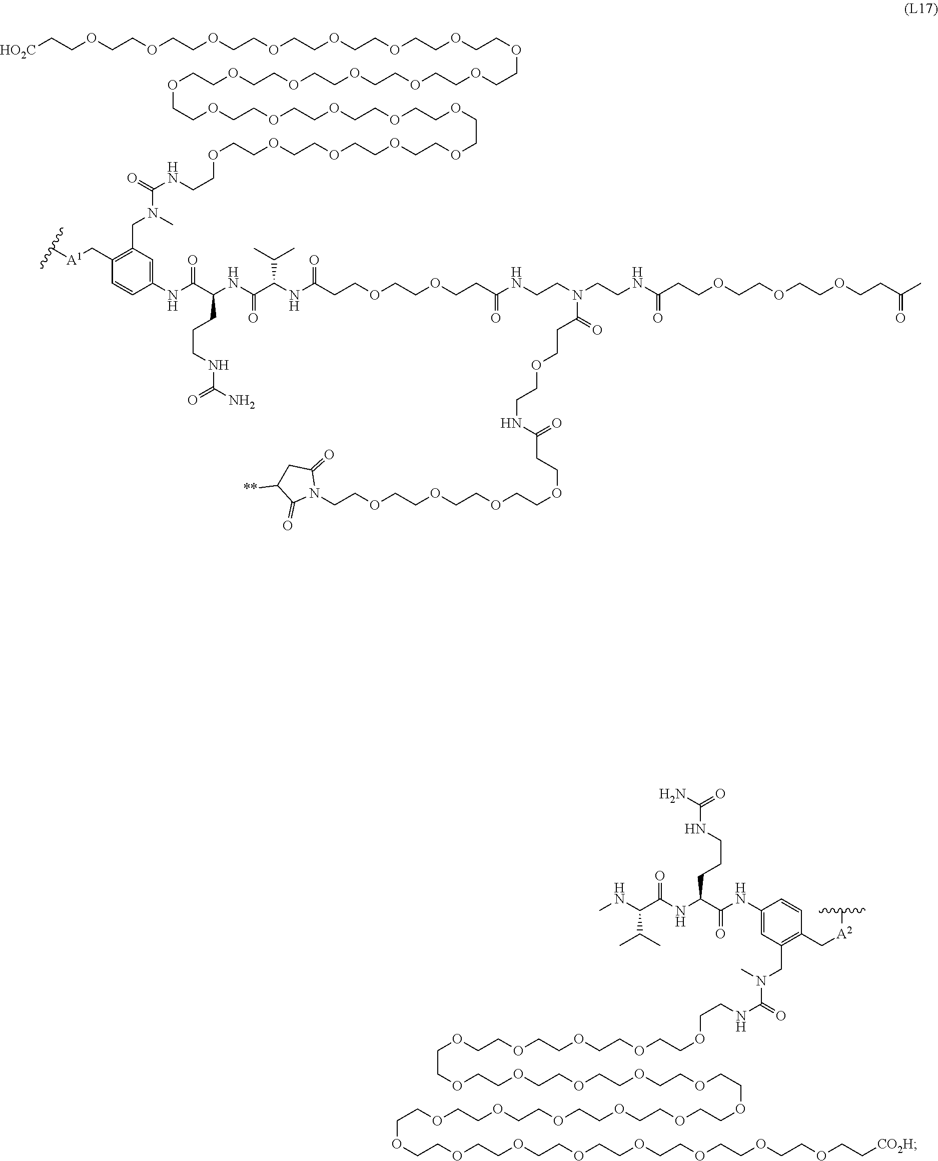

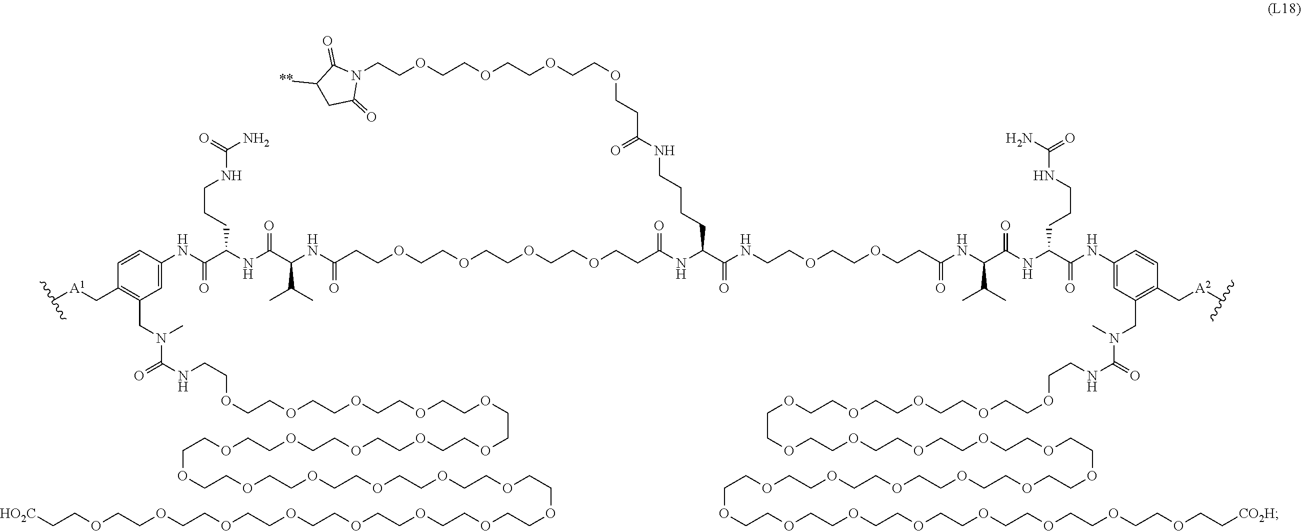

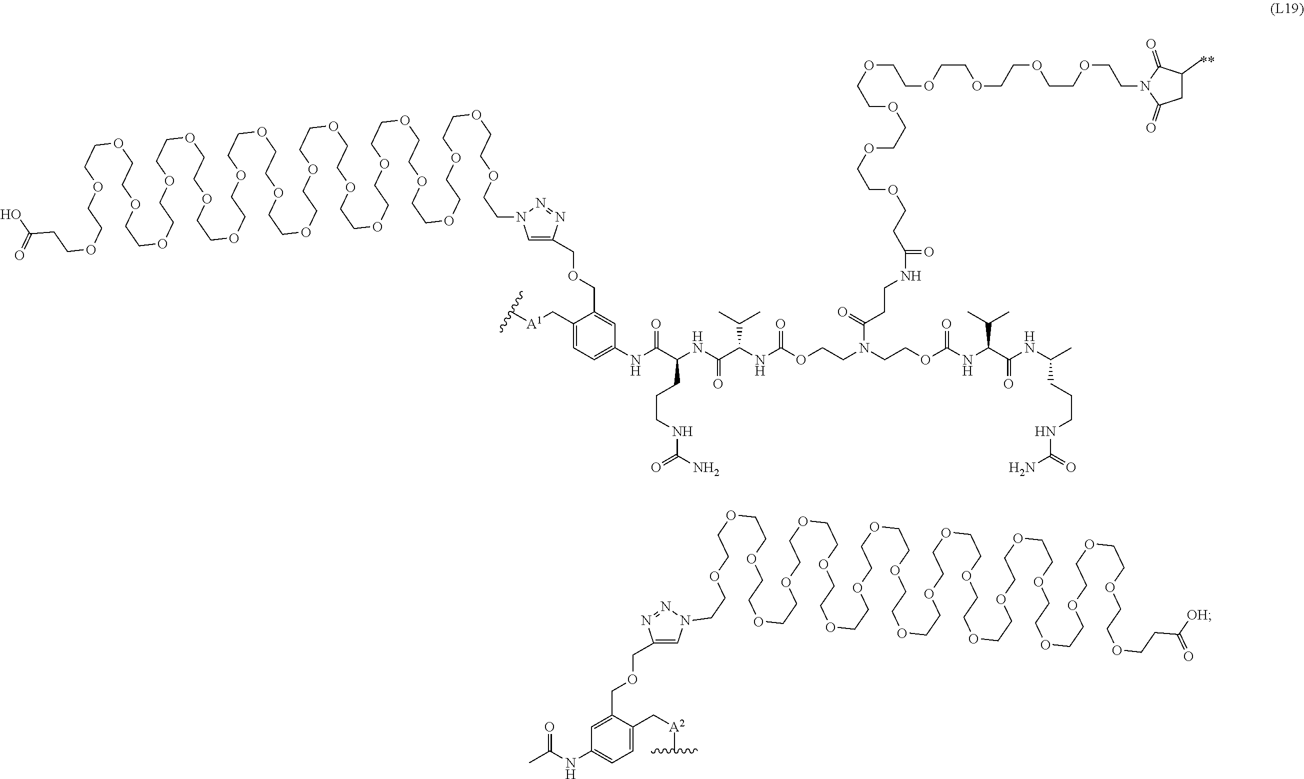

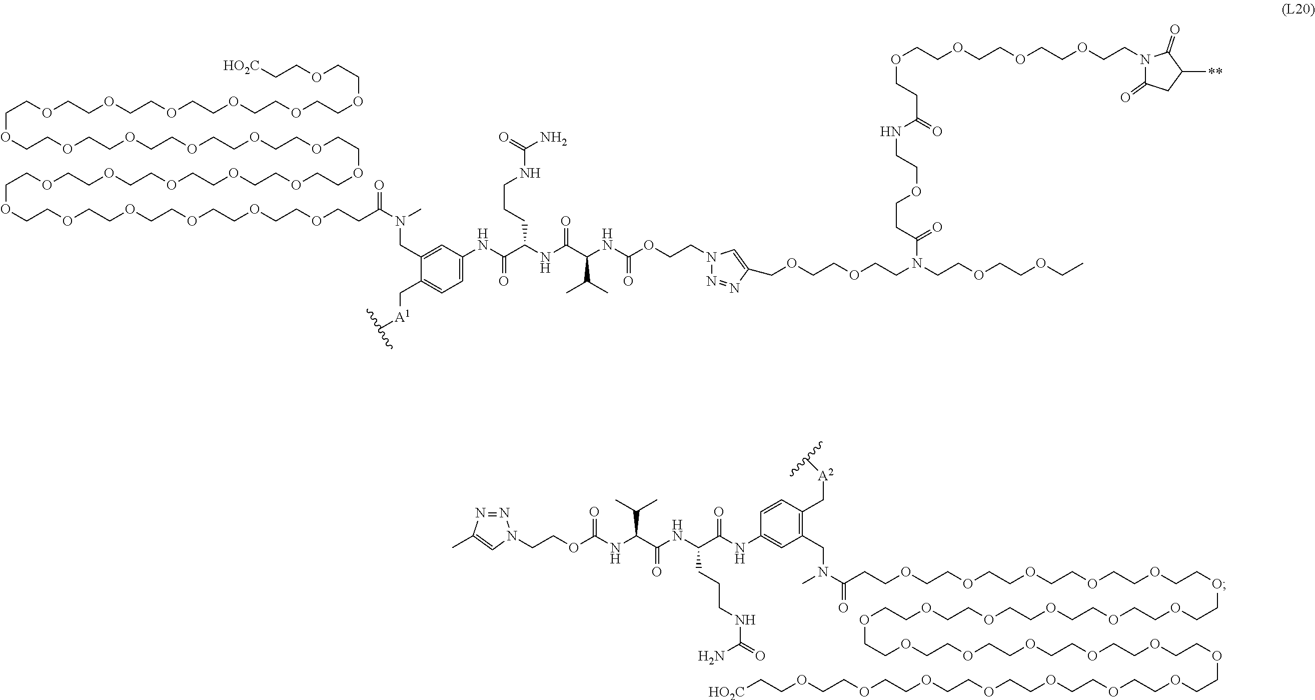

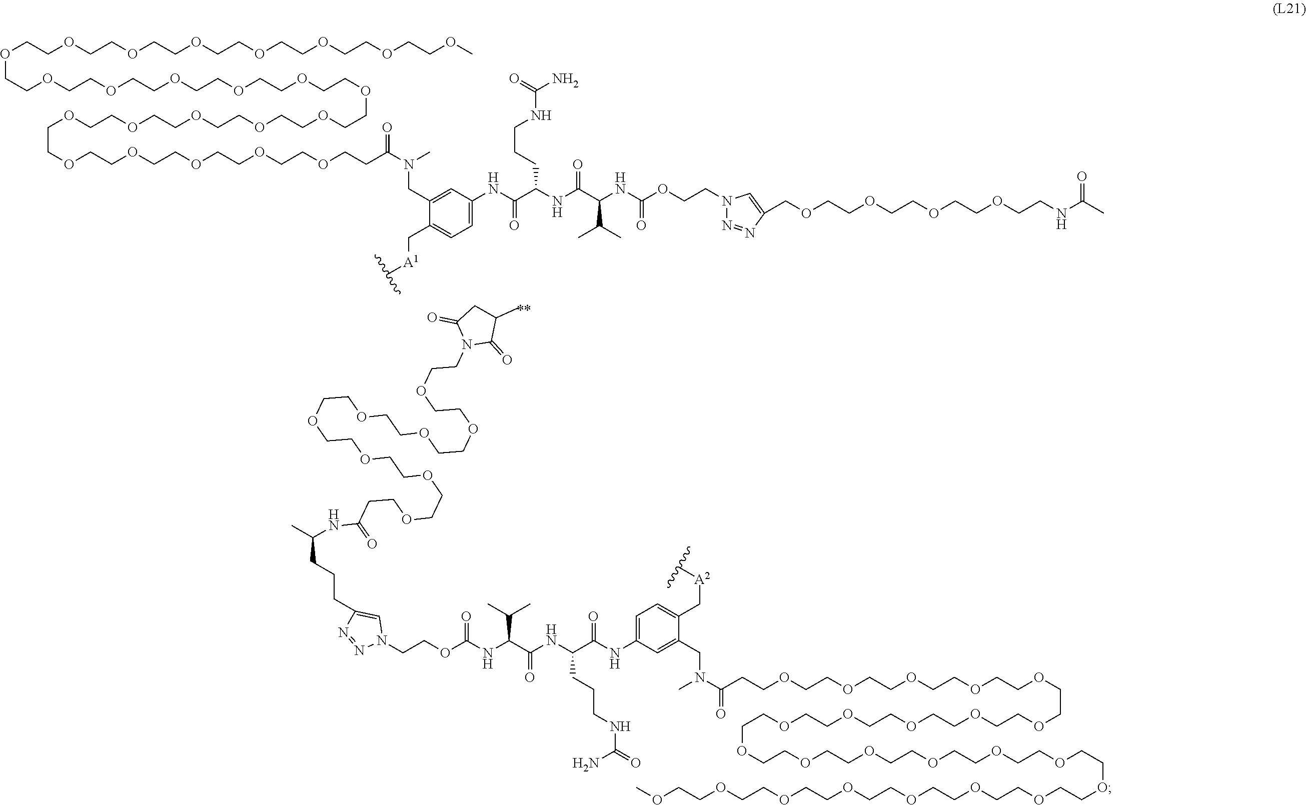

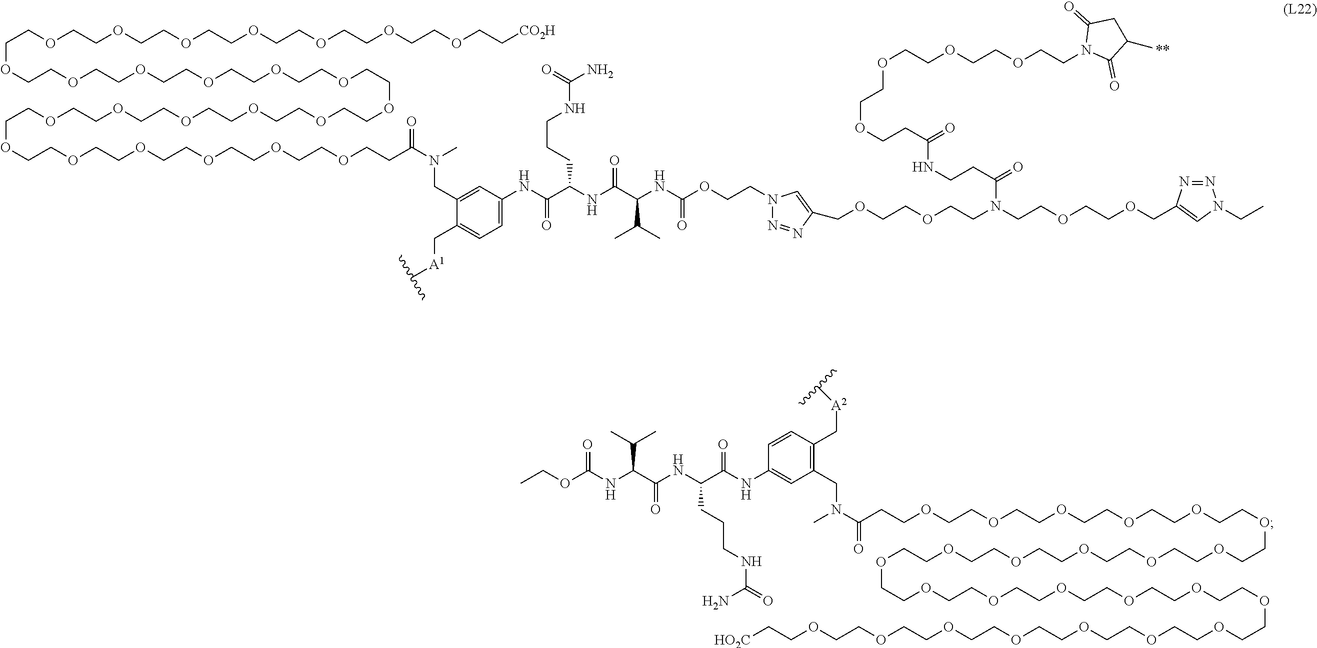

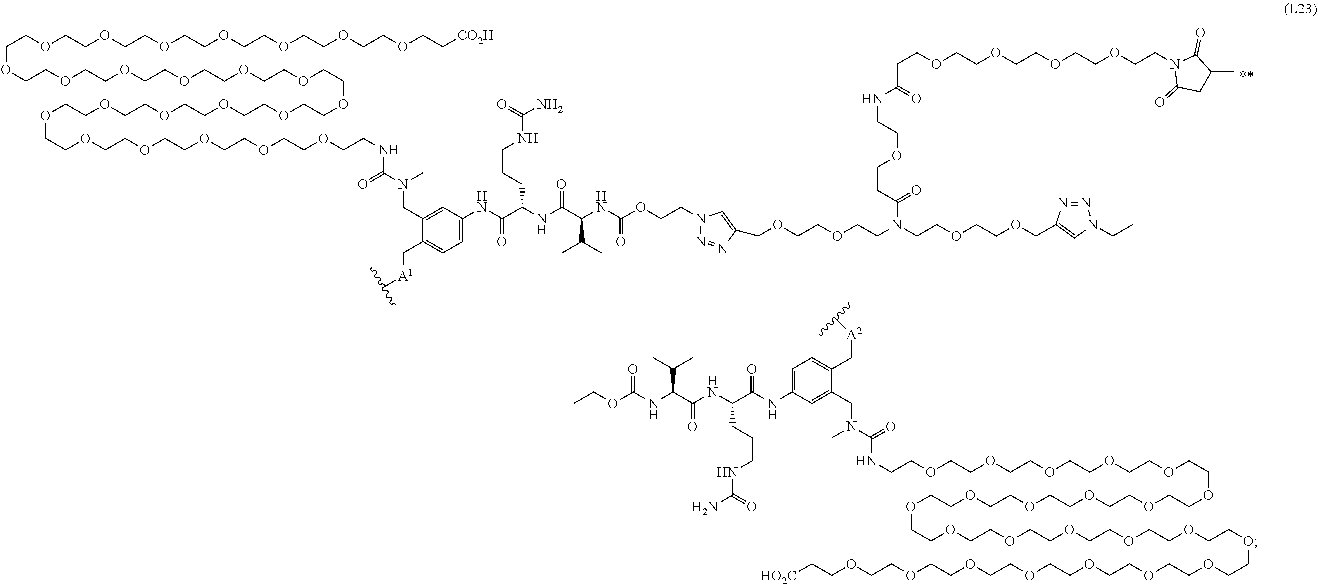

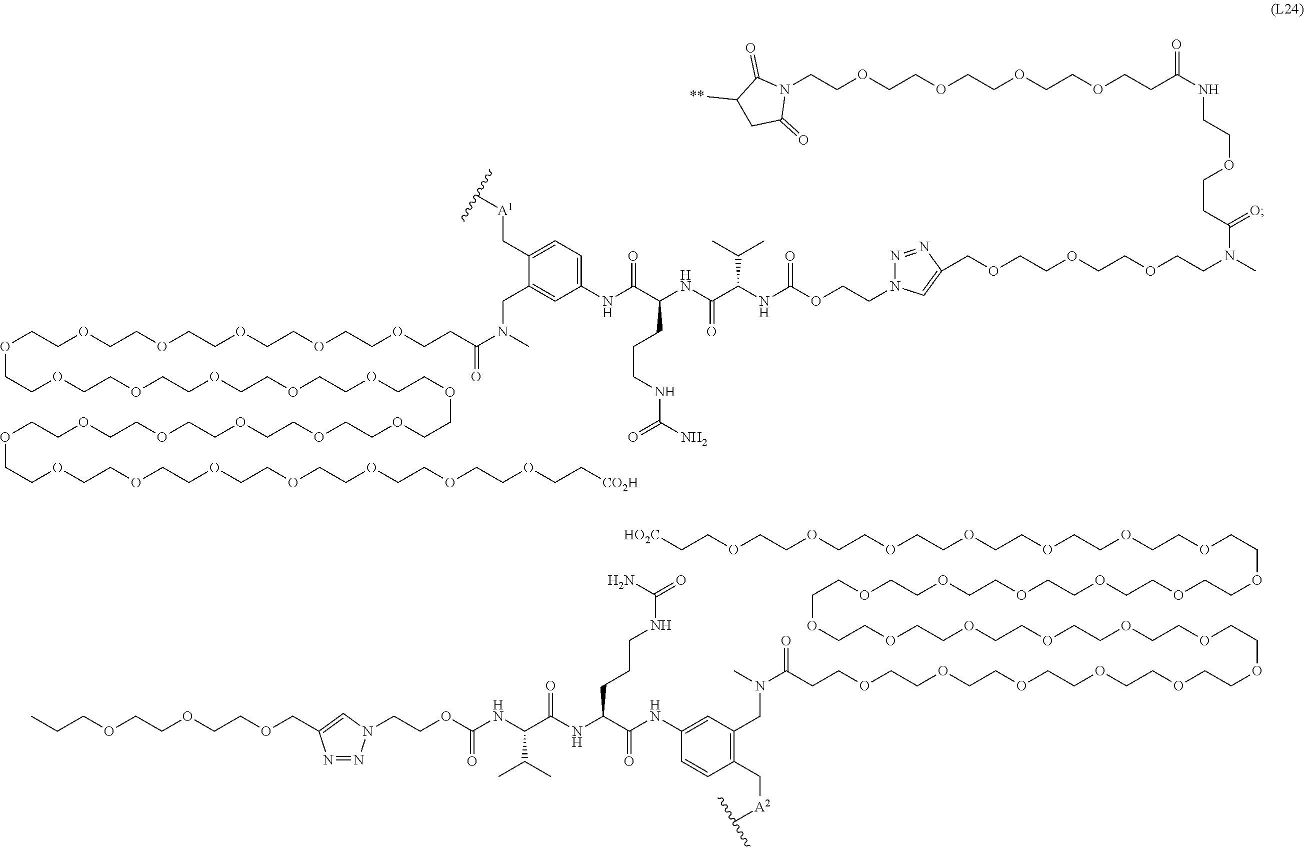

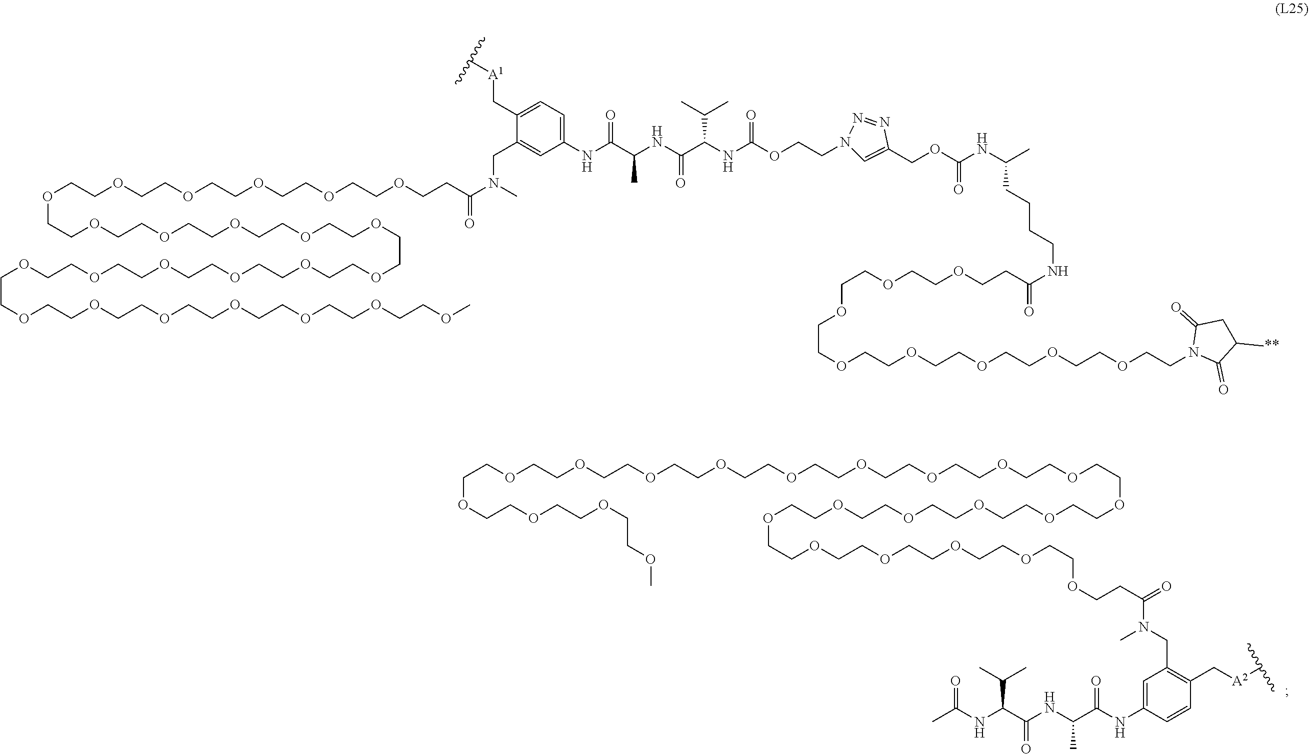

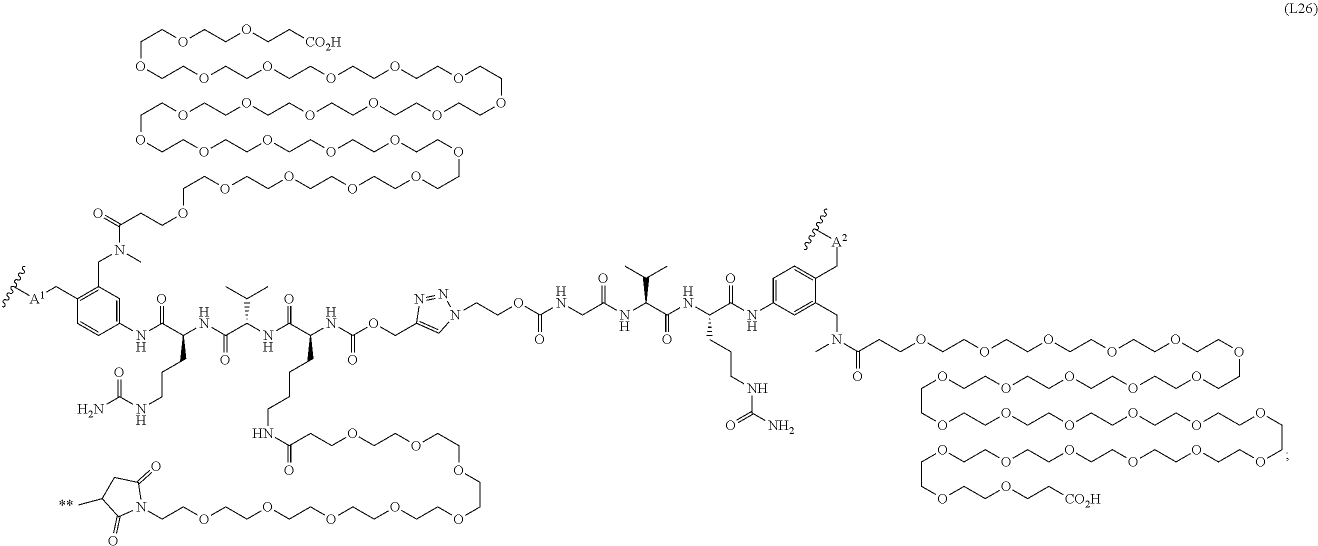

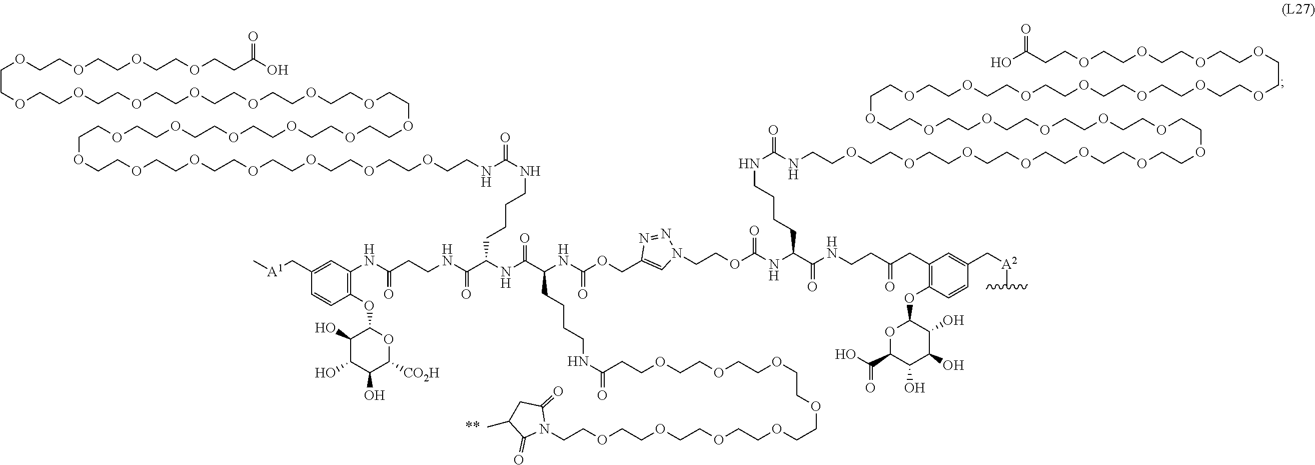

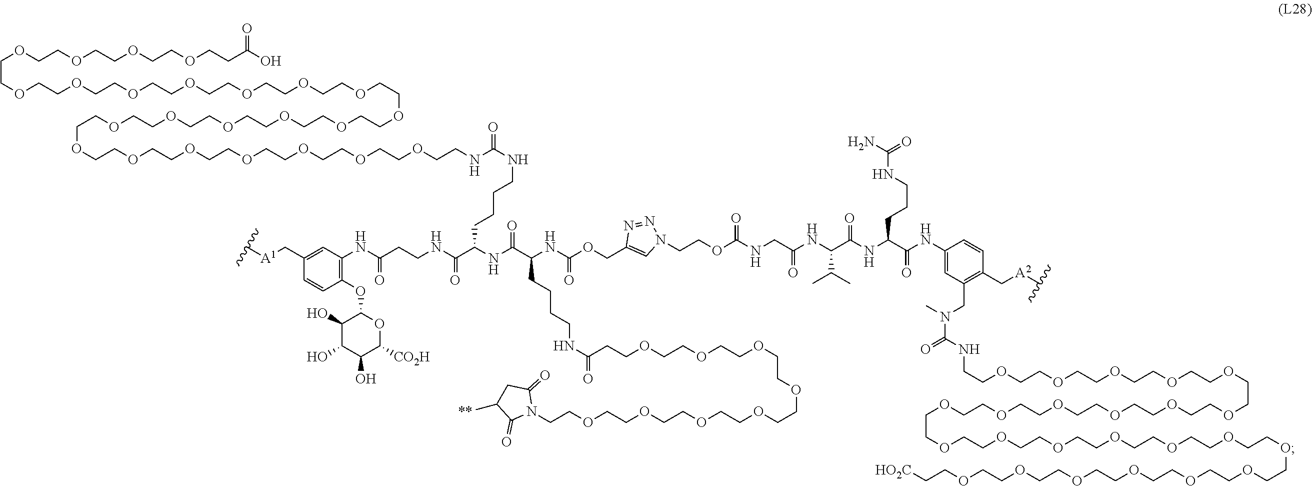

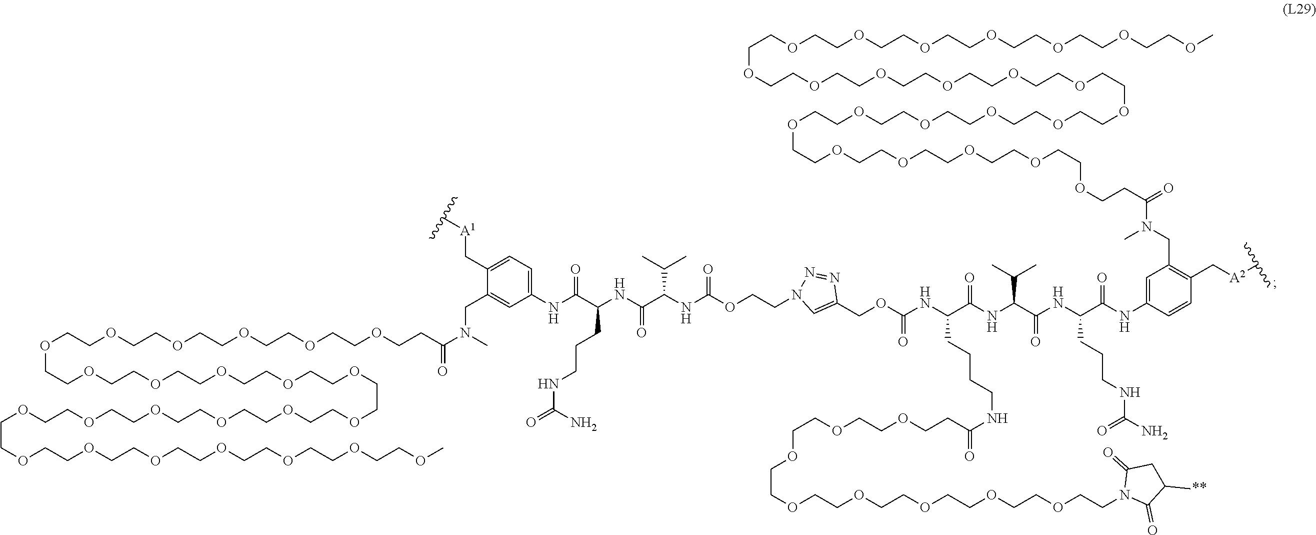

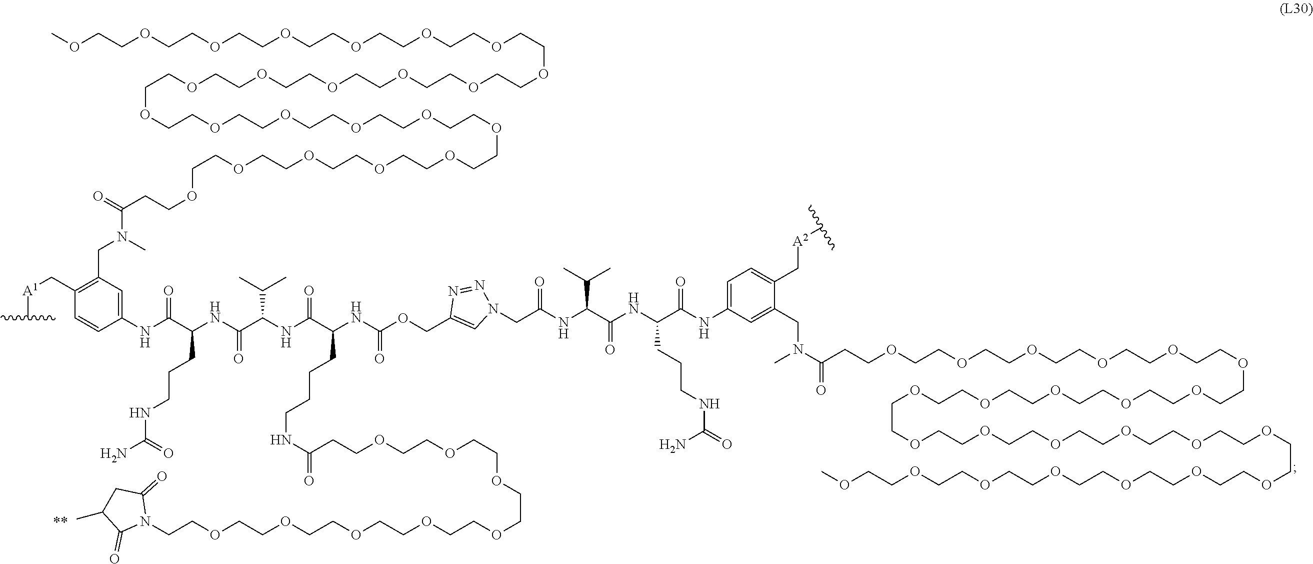

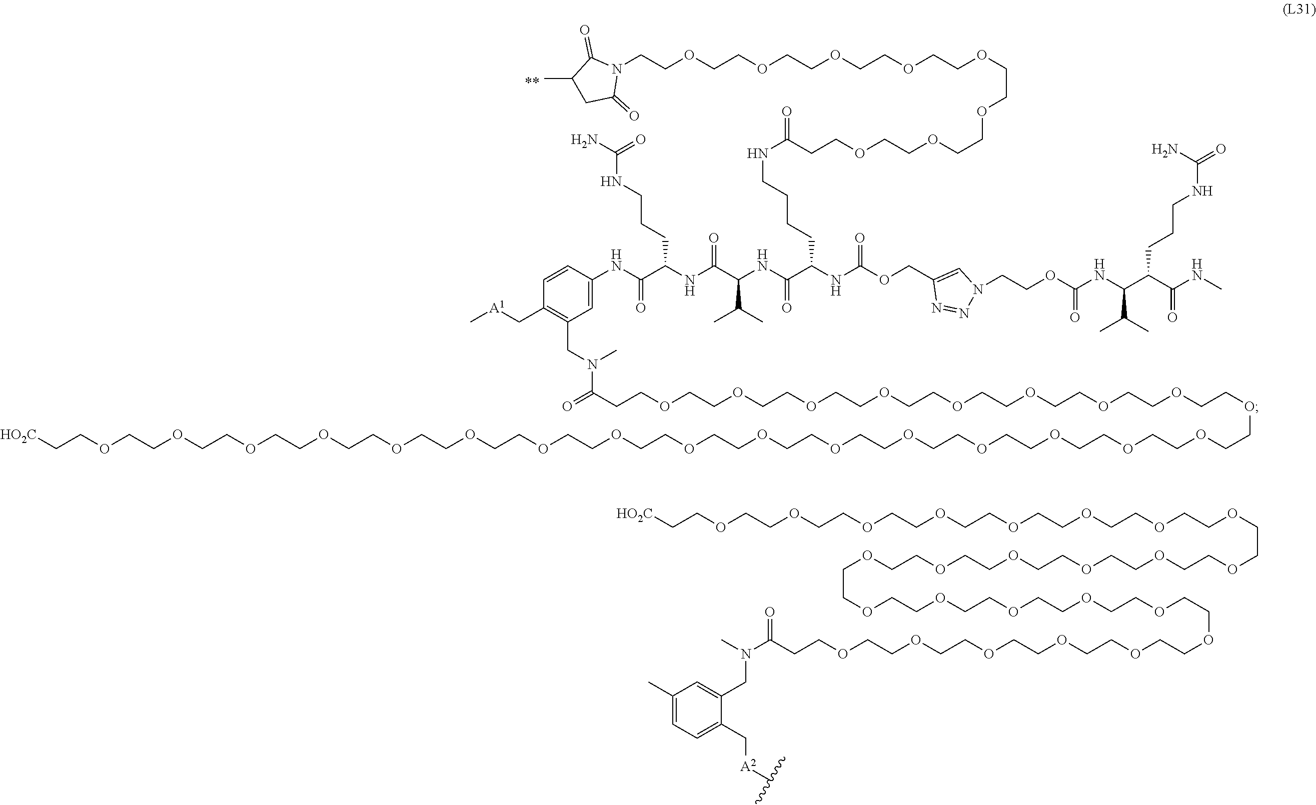

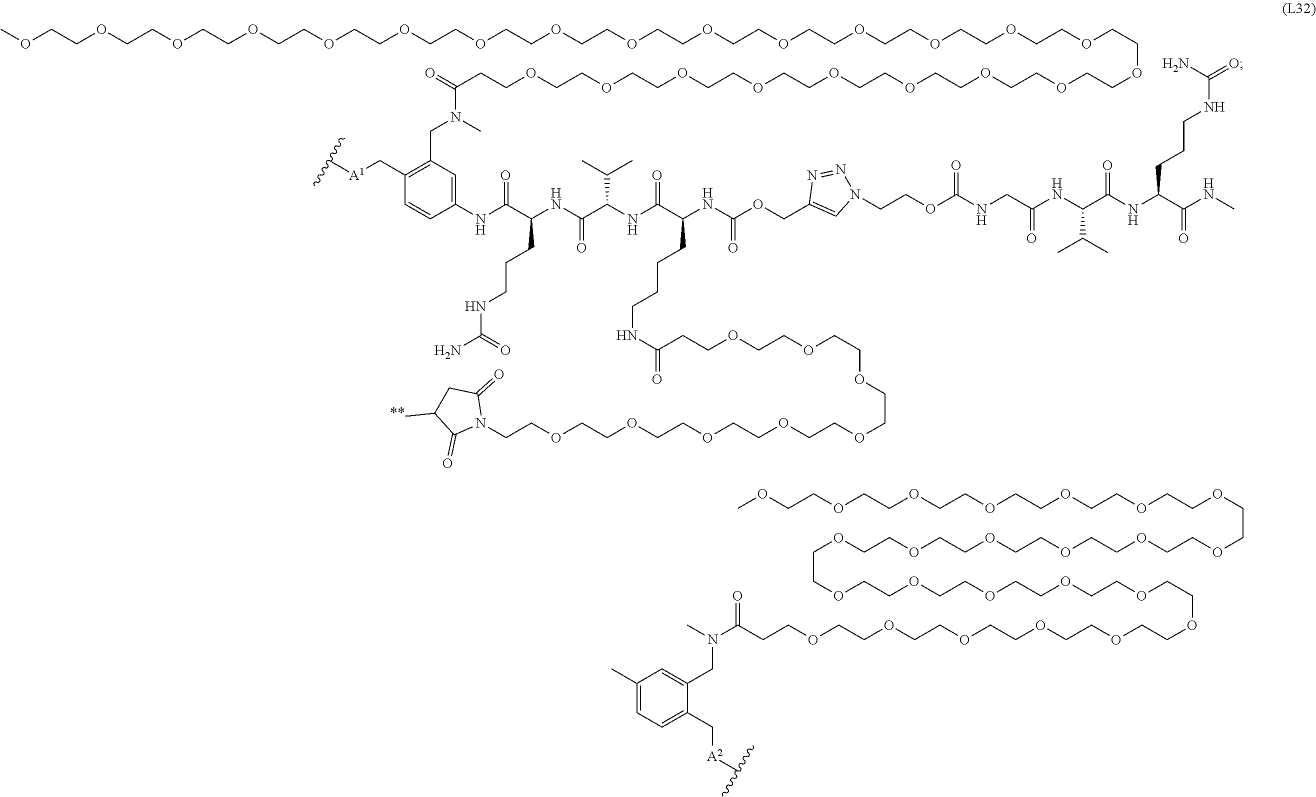

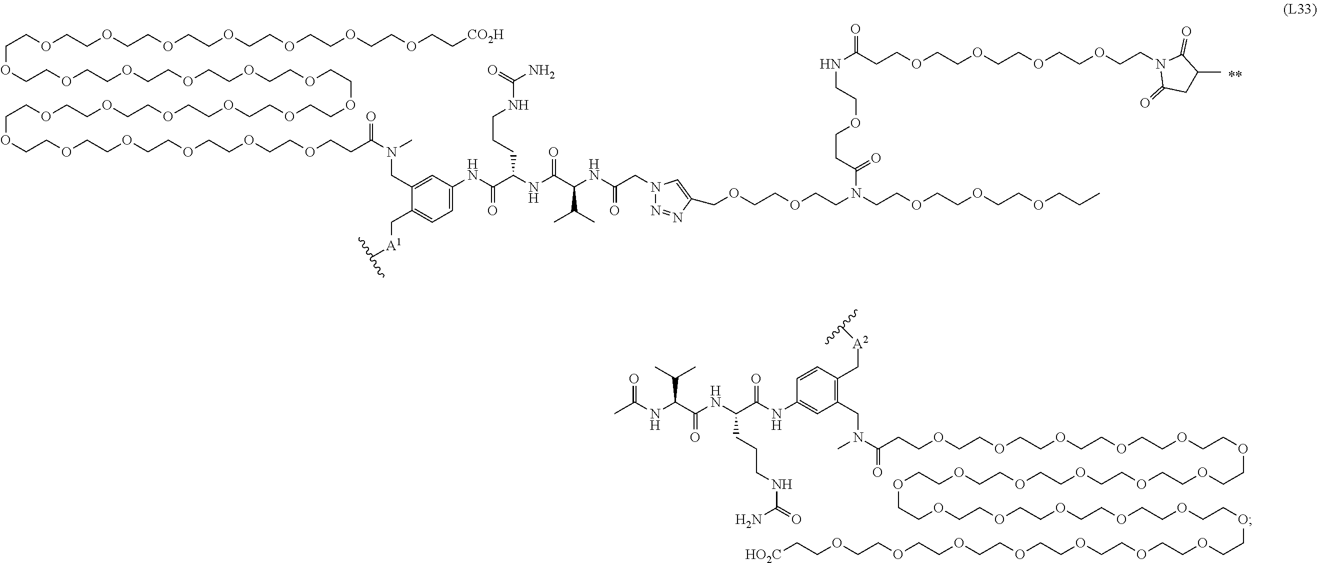

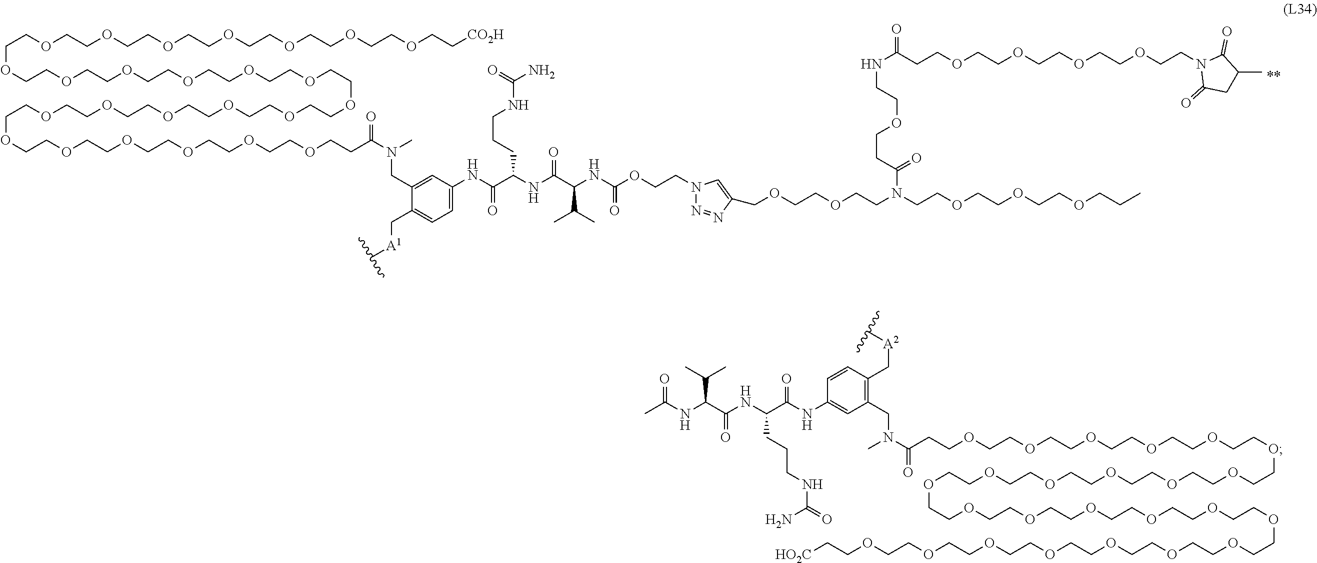

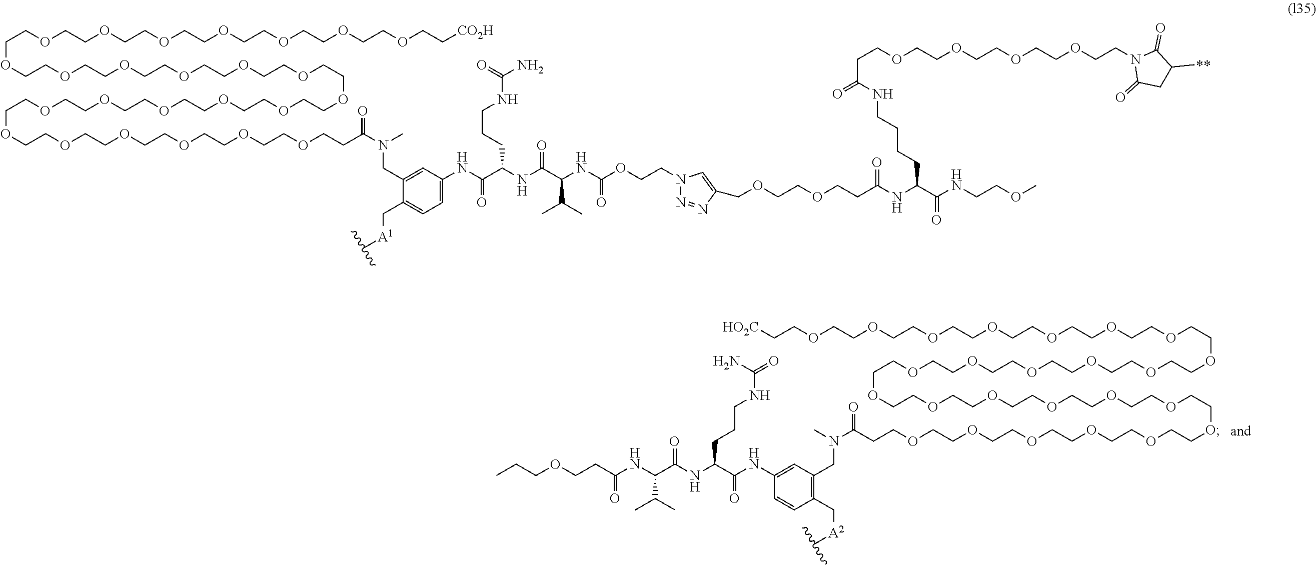

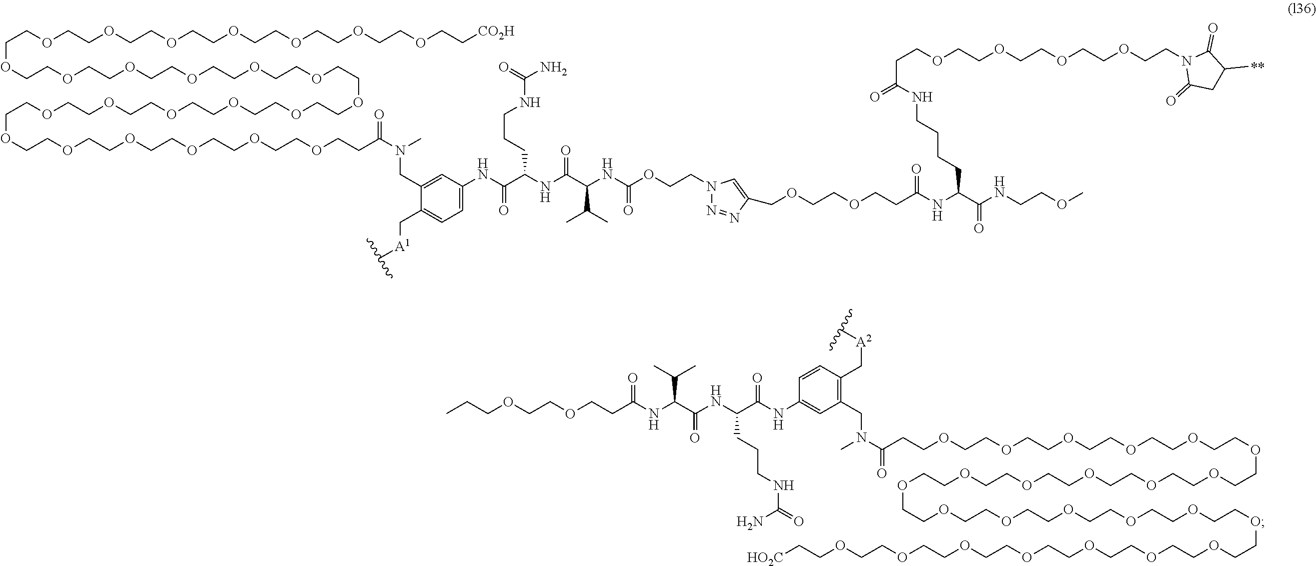

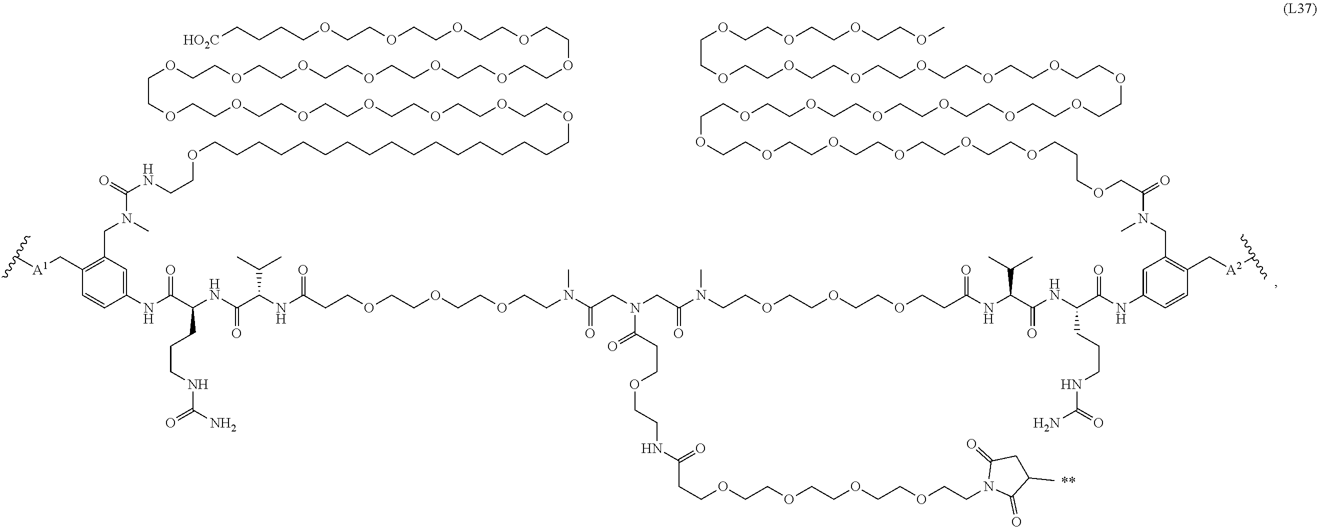

wherein each A¹ or A²independently is a bond, —OC(═O)—* or

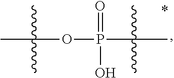 [SMILES: *OP(*)(=O)O]

wherein * indicates the point of attachment to D¹ or D²; [symbol] indicates the point of attachment to the Ab; and [symbol] indicates the point of direct attachment to D¹ or D².

The definitions of the remaining variables are provided in the first embodiment. In some embodiments, each A¹ or A²independently is a bond or —OC(═O)—*. In some embodiments, A¹ and A² are both bond. In some embodiments, A¹ and A² are both —OC(═O)—*. In some embodiments, one of A¹ and A² is a bond and the other is OC(═O)—*.

In a forty-sixth embodiment, the present disclosure provides an antibody-drug conjugate of any one of the second through forty- fifth embodiments, D¹ and D² are each independently a BH3 mimetic. Alternatively, one of D1 and D2 is a BH3 mimetic selected from a Mcl-1 inhibitor, a Bcl-2 inhibitor, and a Bcl-xL inhibitor, and the other is an antineoplastic non-BH3 mimetic selected from topoisomerase 1 inhibitor or an anti-mitotic drug. The definitions of the remaining variables are provided in the second through forty-fifth embodiments or any embodiments described therein. In some embodiments, D¹ is a BH3 mimetic and D² is an antineoplastic non-BH3 mimetic; and the definitions of the remaining variables are provided in the second through forty-fifth embodiments or any embodiments described therein. In some embodiments, D¹ is selected from a Mcl-1 inhibitor, a Bcl-2 inhibitor, and a Bcl-xL inhibitor, and D² is a topoisomerase 1 inhibitor or an anti-mitotic drug. In some embodiments, D¹ is a Bcl-xL inhibitor and D² is a topoisomerase 1 inhibitor. In some embodiments, D¹ is a Bcl-xL inhibitor and D² is an anti-mitotic drug.

In some embodiments, D¹ and/or D² are each independently selected from a Mcl-1 inhibitor, a Bcl-2 inhibitor and a Bcl-xL inhibitor. .

In a forty-seventh embodiment, the present disclosure provides an antibody-drug conjugate of any one of the second through forty-sixth embodiments, wherein D¹ and D² are both (i) a Mcl-1 inhibitor; (ii) a Bcl-2 inhibitor; or (iii) Bcl-xL inhibitor. The definitions of the remaining variables are provided in the second through forty-sixth embodiments or any embodiments described therein.

In a forty-eighth embodiment, the present disclosure provides an antibody-drug conjugate of any one of the second through forty-seventh embodiments, wherein D¹ and D² are the same. The definitions of the remaining variables are provided in the second through forty-sixth embodiments or any embodiments described therein.

In a forty-ninth embodiment, the present disclosure provides an antibody-drug conjugate of any one of the second through forty-seventh embodiments, wherein D¹ and D² are different. The definitions of the remaining variables are provided in the second through forty-seventh embodiments or any embodiments described therein.

In a fiftieth embodiment, the present disclosure provides an antibody-drug conjugate of any one of the second through forty-seventh embodiments, or pharmaceutically acceptable salt thereof, wherein (i) one of D¹ and D² is a Mcl-1 inhibitor and the other is a Bcl-2 inhibitor; (ii) one of D¹ and D²is a Mcl-1 inhibitor and the other is a Bcl-xL inhibitor; or (iii) one of D¹ and D² is a Bcl-2 inhibitor and the other is a Bcl-xL inhibitor. The definitions of the remaining variables are provided in the second through forty-seventh embodiments or any embodiments described therein. Alternatively, the present disclosure provides an antibody-drug conjugate of any one of the second through forty-seventh embodiments, or pharmaceutically acceptable salt thereof, wherein (i) D¹ is a Mcl-1 inhibitor and D² is a Mcl-1 inhibitor; (ii) D¹ is a Mcl-1 inhibitor and D² is a Bcl-2 inhibitor; (iii) D¹ is a Bcl-xL inhibitor and D² is a Bcl-xL inhibitor: (iv) D¹ is a Bcl-xL inhibitor and D² is a Bcl-2 inhibitor; or (v) D¹ is a Bcl-2 inhibitor and D² is a Mcl-1 inhibitor; or (vi) D¹ is a Mcl-1 inhibitor and D² is a Bcl-xL inhibitor. The definitions of the remaining variables are provided in the second through forty-seventh embodiments or any embodiments described therein.

In a fifty-first embodiment, the present disclosure provides an antibody-drug conjugate of any one of the forty-sixth through fiftieth embodiments, or pharmaceutically acceptable salt thereof, the Mcl-1 inhibitor is represented by Formula (I):

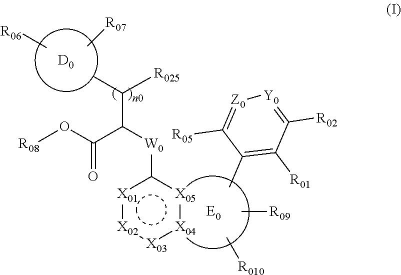

wherein:
Ring D₀ is a cycloalkyl group, a heterocycloalkyl group, an aryl group or a heteroaryl group,
Ring E₀ is a furyl, thienyl or pyrrolyl ring,
X₀₁, X₀₃, X₀₄ and X₀₅ independently of one another are a carbon atom or a nitrogen atom,
X₀₂ is a C—R₀₂₆ group or a nitrogen atom,

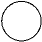

means that the ring is aromatic,
Y₀ is a nitrogen atom or a C—R₀₃ group,
Z₀ is a nitrogen atom or a C-R₀₄ group,
R₀₁ is a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl group, a hydroxy group, a hydroxy(C₁-C₆)alkyl group, a linear or branched (C₁-C₆)alkoxy group, —S—(C₁-C₆)alkyl group, a cyano group, a nitro group, -Cy₀₈, —(C₀-C₆)alkyl-NR₀₁₁R₀₁₁′, —O—(C₁-C₆)alkyl-NR₀₁₁ R₀₁₁′, —O—(C₁-C₆)alkyl-R₀₁₂, —C(O)—OR₀₁₁, —O—C(O)-R₀₁₁, —C(O)—NR₀₁₁ R₀₁₁′, —NR₀₁₁—C(O)—R₀₁₁′, —NR₀₁₁—C(O)—OR₀₁₁′, —(C₁-C₆)alkyl-NR₀₁₁—C(O)—R₀₁₁′, —SO₂—NR₀₁₁R₀₁₁′, or —SO₂-(C₁-C₆)alkyl, R₀₂, R₀₃, R₀₄ and R₀₅ independently of one another are a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl, a hydroxy group, a hydroxy(C₁-C₆)alkyl group, a linear or branched (C₁-C₆)alkoxy group, a —S—(C₁-C₆)alkyl group, a cyano group, a nitro group, —(C₀-C₆)alkyl-NR₀₁₁R₀₁₁′, -O-Cy₀₁, —(C₀-C₆)alkyl-Cy₀₁, -(C₂-C₆)alkenyl-Cy₀₁, —(C₂-C₆)alkynyl-Cy₀₁, —O—(C₁-C₆)alkyl-NR₀₁₁R₀₁₁′, —O—(C₁-C₆)alkyl-R₀₃₁,—O—(C₁-C₆)alkyl-R₀₁₂, —C(O)—OR₀₁₁, —O—C(O)—R₀₁₁, —C(O)—NR₀₁₁ R₀₁₁′, —NR₀₁₁—C(O)—R₀₁₁′, —NR₀₁₁—C(O)—OR₀₁₁′, —(C₁-C₆)alkyl-NR₀₁—C(O)—R₀₁₁′, —SO₂—NR₀₁₁R₀₁₁′, or —SO₂—(C₁-C₆)alkyl,

or the pair (R₀₁₁, R₀₂), (R₀₂, R₀₃), (R₀₃, R₀₄), or (R₀₄, Ros) together with the carbon atoms to which they are attached form an aromatic or non-aromatic ring containing 5 to 7 ring members, which optionally contains 1 to 3 heteroatoms selected from O, S and N, wherein the resulting ring is optionally substituted by 1 or 2 groups selected from halogen, linear or branched (C₁-C₆)alkyl, (C₀-C₆)alkyl-NR₀₁₁R₀₁₁′, —NR₀₁₃R₀₁₃′, —(C₀-C₆)alkyl-Cy₀₁ or oxo, R₀₆ and R₀₇ independently of one another are a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl, a hydroxy group, a linear or branched (C₁-C₆)alkoxy group, a —S—(C₁-C₆)alkyl group, a cyano group, a nitro group, —(C₀-C₆)alkyl-NR₀₁₁R₀₁₁′, —O—(C₁-C₆)alkyl-NR₀₁₁R₀₁₁′, —O-Cy₀₁, —(C₀-C₆)alkyl-Cy₀₁, —(C₂-C₆)alkenyl-Cy₀₁, -(C₂-C₆)alkynyl-Cy₀₁, —O—(C₁-C₆)alkyl-R₀₁₂, —C(O)—OR₀₁₁, —O—C(O)—R₀₁₁, —C(O)—NR₀₁₁ R₀₁₁′, —NR₀₁₁—C(O)—R₀₁₁′, —NR₀₁₁—C(O)—OR₀₁₁′, -(C₁-C₆)alkyl-NR₀₁₁—C(O)—R₀₁₁′, —SO₂—NR₀₁R₀₁₁′, or —SO₂—(C₁-C₆)alkyl, or the pair (R₀₆, R₀₇), when fused with the two adjacent carbon atoms, together with the carbon atoms to which they are attached form an aromatic or non-aromatic ring containing 5 to 7 ring members, which optionally contains 1 to 3 heteroatoms selected from O, S and N, wherein the resulting ring is optionally substituted by a linear or branched (C₁-C₆)alkyl group, —NR₀₁₃R₀₁₃′, —(C₀-C₆)alkyl-Cy₀₁ or an oxo,
W₀ is a —CH₂— group, a —NH— group or an oxygen atom,
R₀₈ is a hydrogen atom, a linear or branched (C₁-C₅)alkyl group, a —CHR₀ₐR_{0b} group, an aryl group, a heteroaryl group, an aryl(C₁-C₆)alkyl group, or a heteroaryl(C₁-C₆)alkyl group,
R₀₉ is a hydrogen atom, a linear or branched (C₁-C₆)alkyl group, a linear or branched (C₂-C₆)alkenyl group, a linear or branched (C₂-C₆)alkynyl group, —Cy₀₂, —(C₁-C₆)alkyl-Cy₀₂, —(C₂-C₆)alkenyl-Cy₀₂, —(C₂-C₆)alkynyl-Cy₀₂, —Cy₀₂—Cy₀₃, —(C₂-C₆)alkynyl-O—Cy₀₂, —Cy₀₂—(C₀-C₆)alkyl-O—(C₀-C₆)alkyl-Cy₀₃, a halogen atom, a cyano group, —C(O)—R₀₁₄, or —C(O)—NR₀₁₄R₀₁₄′,
R₀₁₀ is a hydrogen atom, a linear or branched (C₁-C₆)alkyl group, a linear or branched (C₂-C₆)alkenyl group, a linear or branched (C₂-C₆)alkynyl group, an aryl(C₁-C₆)alkyl group, a (C₁-C₆)cycloalkylalkyl group, a linear or branched (C₁-C₆)haloalkyl, or —(C₁-C₆)alkyl-O—Cy₀₄, or the pair (R₀₉, R₀₁₀), when fused with the two adjacent carbon atoms, together with the carbon atoms to which they are attached form an aromatic or non-aromatic ring containing 5 to 7 ring members, which optionally contains 1 to 3 heteroatoms selected from O, S and N,
R₀₁₁ and R₀₁₁′ independently of one another are a hydrogen atom, an optionally substituted linear or branched (C₁-C₆)alkyl group, or —(C₀-C₆)alkyl-Cy₀₁, or the pair (R₀₁₁, R₀₁₁′) together with the nitrogen atom to which they are attached form an aromatic or non-aromatic ring containing 5 to 7 ring members, which optionally contains, in addition to the nitrogen atom, 1 to 3 heteroatoms selected from O, S, and N, wherein the N atom may be substituted by 1 or 2 groups selected from a linear or branched (C₁-C₆)alkyl group, and wherein one or more of the carbon atoms of the linear or branched (C₁-C₆)alkyl group is optionally deuterated,
R₀₁₂ is —Cy₀₈, —Cy₀₅—(C₀-C₆)alkyl-O—(C₀-C₆)alkyl-Cy₀₆, —Cy₀₅—(C₀-C₆)alkyl-Cy₀₆, —Cy₀₅—(C₀-C₆)alkyl-NR₀₁₁—(C₀-C₆)alkyl-Cy₀₆, —Cy₀₅—Cy₀₆—O—(C₀-C₆)alkyl-Cy₀₇, —Cy₀₅—(C₀-C₆)alkyl-O-(C₀-C₆)alkyl-Cy₀₉, —Cy₀₅—(C₀-C₆)alkyl-Cy₀₉, —NH—C(O)—NH—R₀₁₁, —Cy₀₅—(C₀-C₆)alkyl-NR₀₁₁—(C₀-C₆)alkyl-Cy₀₉, —C(O)—NR₀₁₁R₀₁₁′, —NR₀₁₁R₀₁₁′, —OR₀₁₁, —NR₀₁₁—C(O)—R₀₁₁′, —O—(C₁-C₆)alkyl-OR₀₁₁, —SO₂—R₀₁₁, —C(O)—OR₀₁₁,
R₀₁₃, R₀₁₃′, R₀₁₄ and R₀₁₄′ independently of one another are a hydrogen atom, or an optionally substituted linear or branched (C₁-C₆)alkyl group,
R₀ₐ is a hydrogen atom or a linear or branched (C₁-C₆)alkyl group,
R_{0b} is a —O—C(O)—O—R_{0c} group, a —O—C(O)—NR_{0c}R_{0c}′ group, or a —O—P(O)(OR_{0c})₂ group,
R_{0c} and R_{0c}′ independently of one another are a hydrogen atom, a linear or branched (C₁-C₅)alkyl group, a cycloalkyl group, a (C₁-C₆)alkoxy(C₁-C₆)alkyl group, or a (C₁-C₆)alkoxycarbonyl(C₁-C₆)alkyl group, or the pair (R_{0c}, R_{0c}′) together with the nitrogen atom to which they are attached form a non-aromatic ring composed of from 5 to 7 ring members, which may contain in addition to the nitrogen atom from 1 to 3 heteroatoms selected from oxygen and nitrogen, wherein the nitrogen is optionally substituted by a linear or branched (C₁-C₆)alkyl group,
Cy₀₁, Cy₀₂, Cy₀₃, Cy₀₄, Cy₀₈, Cy₀₆, Cy₀₇, Cy₀₈ and Cy₀₁₀ independently of one another, are a cycloalkyl group, a heterocycloalkyl group, an aryl group or a heteroaryl group, each of which is optionally substituted,
Cy₀₉ is

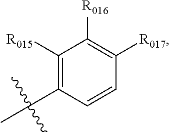

or Cy₀₉ is a heteroaryl group which is substituted by a group selected from —O—P(O)(OR₀₂₀)₂; —O—P(O)(OM⁺)₂; —(CH₂)ₚ₀—O—(CHR₀₁₈—CHR₀₁₉—O)_{q0}—R₀₂₀; hydroxy; hydroxy(C₁-C₆)alkyl; —(CH₂)ᵣ₀—U₀—(CH₂)ₛ₀heterocycloalkyl; and —U₀—(CH₂)_{q0}—NR₀₂₁R₀₂₁′,
R₀₁₅ is a hydrogen atom; a —(CH₂)ₚ₀—O—(CHR₀₁₈—CHR₀₁₉—O)_{q0}—R₀₂₀ group; a linear or branched (C₁-C₆)alkoxy(C₁-C₆)alkyl group; a —U₀—(CH₂)_{q0}—NR₀₂₁R₀₂₁′ group; or a —(CH₂)ᵣ₀—U₀—(CH₂)ₛ₀heterocycloalkyl group,
R₀₁₆ is a hydrogen atom; a hydroxy group; a hydroxy(C₁-C₆)alkyl group; a —(CH₂)ᵣ₀—U₀—(CH₂)ₛ₀heterocycloalkyl group; a (CH₂)ᵣ₀—U₀—V₀—O—P(O)(OR₀₂₀)₂ group; a —O—P(O)(O⁻M⁺)₂ group; a —O—S(O)₂OR₀₂₀ group; a —S(O)₂OR₀₂₀ group; a —(CH₂)ₚ₀—O—(CHR₀₁₈—CHR₀₁₉—O)_{q0}—R₀₂₀ group; a —(CH₂)ₚ₀—O—C(O)—NR₂₂R₀₂₃ group; or a —U₀—(CH₂)_{q0}—NR₀₂₁R₀₂₁′ group, R_{01y} is a hydrogen atom; a —(CH₂)ₚ₀—O—(CHR₀₁₈—CHR₀₁₉—O)_{q0}—R₀₂₀ group; a —CH₂—P(O)(OR₀₂₀)₂ group, a —O—P(O)(OR₀₂₀)₂ group; a —O—P(O)(O⁻M⁺)₂ group; a hydroxy group; a hydroxy(C₁-C₆)alkyl group; a —(CH₂)ᵣ₀—U₀—(CH₂)ₛ₀heterocycloalkyl group; a —U₀—(CH₂)_{q0}—NR₀₂₁R₀₂₁′ group; or an aldonic acid,
M⁺ is a pharmaceutically acceptable monovalent cation,
U₀ is a bond or an oxygen atom,
V₀ is a —(CH₂)ₛ₀ group or a —C(O)— group,
R₀₁ₛ is a hydrogen atom or a (C₁-C₆)alkoxy(C₁-C₆)alkyl group,
R₀₁₉ is a hydrogen atom or a hydroxy(C₁-C₆)alkyl group,
R₀₂₀ is a hydrogen atom or a linear or branched (C₁-C₆)alkyl group,
R₀₂₁ and R₀₂₁′ independently of one are a hydrogen atom, a linear or branched (C₁-C₆)alkyl group, or a hydroxy(C₁-C₆)alkyl group, or the pair (R₀₂₁, R₀₂₁′) together with the nitrogen atom to which they are attached form an aromatic or non-aromatic ring containing 5 to 7 ring members, which optionally contains, in addition to the nitrogen atom, 1 to 3 heteroatoms selected from O, S and N, wherein the resulting ring is optionally substituted by a hydrogen atom or a linear or branched (C₁-C₆)alkyl group,
R₀₂₂ is a (C₁-C₆)alkoxy(C₁-C₆)alkyl group, a —(CH₂)ₚ₀—NR₀₂₄R₀₂₄′ group, or a —(CH₂)ₚ₀—O—(CHR₀₁₈—CHR₀₁₉—O)_{q0}—R₂₀ group,
R₀₂₃ is a hydrogen atom or a (C₁-C₆)alkoxy(C₁-C₆)alkyl group, or the pair (R₀₂₂, R₀₂₃) together with the nitrogen atom to which they are attached form an aromatic or non-aromatic ring containing 5 to 18 ring members, which optionally contains, in addition to the nitrogen atom, 1 to 5 heteroatoms selected from O, S and N, wherein the resulting ring is optionally substituted by a hydrogen atom, a linear or branched (C₁-C₆)alkyl group or a heterocycloalkyl group,
R₀₂₄ and R₀₂₄′ independently of one another are a hydrogen atom or a linear or branched (C₁-C₆)alkyl group, or the pair (R₀₂₄, R₀₂₄′) together with the nitrogen atom to which they are attached form an aromatic or non-aromatic ring composed of from 5 to 7 ring members, which may contain in addition to the nitrogen atom from 1 to 3 heteroatoms selected from O, S and N, and wherein the resulting ring is optionally substituted by a hydrogen atom or a linear or branched (C₁-C₆)alkyl group,
R₀₂₅ is a hydrogen atom, a hydroxy group, or a hydroxy(C₁-C₆)alkyl group,
R₀₂₆ is a hydrogen atom, a halogen atom, a linear or branched (C₁-C₆)alkyl group, or a cyano group,
R₀₂₇ is a hydrogen atom or a linear or branched (C₁-C₆)alkyl group,
R₀₂₈ is a —O—P(O)(O⁻)(O⁻) group, a —O—P(O)(O⁻)(OR₀₃₀) group, a —O—P(O)(OR₀₃₀)(OR₀₃₀′) group, a —(CH₂)ₚ₀—O—SO₂—O group, a —(CH₂)ₚ₀—SO₂—O group, a —(CH₂)ₚ₀—O—SO₂—OR₀₃₀ group, —Cy₀₁₀, a —(CH₂)ₚ₀—SO₂—OR₀₃₀ group, a —O—C(O)—R₀₂₉ group, a —O—C(O)—OR₀₂₉ group or a —O—C(O)—NR₂₉R₀₂₉′ group;
R₀₂₉ and R₀₂₉′ independently of one another are a hydrogen atom, a linear or branched (C₁-C₆)alkyl group or a linear or branched amino(C₁-C₆)alkyl group,
R₀₃₀ and R₀₃₀′ independently of one another are a hydrogen atom, a linear or branched (C₁-C₆)alkyl group or an aryl(C₁-C₆)alkylgroup,

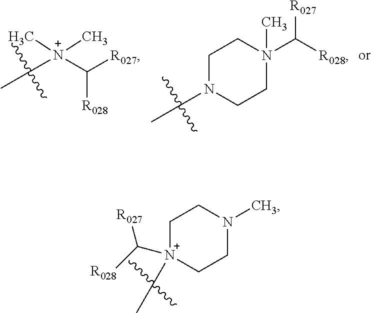

wherein the ammonium optionally exists as a
zwitterionic form or has a monovalent anionic counterion,
n₀ is an integer equal to 0 or 1,
p₀ is an integer equal to 0, 1, 2, or 3,
q₀ is an integer equal to 1, 2, 3 or 4,
r₀ and so are independently an integer equal to 0 or 1;
wherein, at most, one of the R₀₃, R₀₉, or R₀₁₂ groups, if present, is covalently attached to the linker, and
wherein the valency of an atom is not exceeded by virtue of one or more substituents bonded thereto,
or an enantiomer, diastereoisomer, atropisomer, deuterated derivative, and/or pharmaceutically acceptable salt of any of the foregoing.

The definitions of the remaining variables are provided in the forty-sixth through fiftieth embodiments or any embodiments described therein.

In a fifty-second embodiment, the present disclosure provides an antibody-drug conjugate of the fifty-first embodiment, wherein Cy₀₁, Cy₀₂, Cy₀₃, Cy₀₄, Cy₀₈, Cy₀₆, Cy₀₇, Cy₀₈ and Cy₀₁₀, independently of one another, is a cycloalkyl group, a heterocycloalkyl group, an aryl group or a heteroaryl group, each of which is optionally substituted by one or more groups selected from halo; —(C₁-C₆)alkoxy; —(C₁-C₆)haloalkyl; —(C₁-C₆)haloalkoxy; —(CH₂)ₚ₀—O—SO₂—OR₀₃₀; —(CH₂)ₚ₀—SO₂—OR₀₃₀; —O—P(O)(OR₀₂₀)₂; —O—P(O)(O⁻M⁺)₂; —CH₂—P(O)(OR₀₂₀)₂; —(CH₂)ₚ₀—O—(CHR₀₉—CHR₀₁₉—O)_{q0}—R₀₂₀; hydroxy; hydroxy(C₁-C₆)alkyl; —(CH₂)ᵣ₀—U₀—(CH₂)ₛ₀—heterocycloalkyl; or —U₀—(CH₂)_{q0}—NR₀₂₁R₀₂₁′.

The definitions of the remaining variables are provided in the fifty-first embodiment or any embodiments described therein.

In a fifty-third embodiment, the present disclosure provides an antibody-drug conjugate of the fifty-first embodiment, wherein the Mcl-1 inhibitor is presented by Formula (IA):

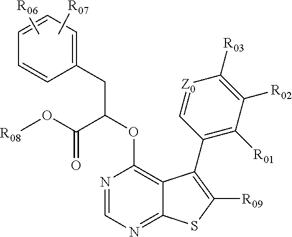

wherein:
Z₀ is a nitrogen atom or a C-R₀₄ group,
R₀₁ is a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl group, a hydroxy group, a linear or branched (C₁-C₆)alkoxy group, a —S—(C₁-C₆)alkyl group, a cyano group, —Cy₀₈, —NR₀₁₁R₀₁₁′,
R₀₂, R₀₃ and R₀₄ independently of one another are a hydrogen atom, a halogen atom, a linear or branched (C₁-C₆)alkyl group, a linear or branched (C₂-C₆)alkenyl group, a linear or branched (C₂-C₆)alkynyl group, a linear or branched (C-C₆)haloalkyl, a hydroxy group, a linear or branched (C₁-C₆)alkoxy group, a —S—(C₁-C₆)alkyl group, a cyano group, a nitro group, —(C₀-C₆)alkyl-NR₀₁₁R₀₁₁′, —O—Cy₀₁, —(C₀-C₆)alkyl-Cy₀₁, —(C₂-C₆)alkenyl-Cy₀₁, —(C₂-C₆)alkynyl-Cy₀₁, —O—(C₁-C₆)alkyl-NR₀₁ R₀₁₁′, —O—(C₁-C₆)alkyl-R₀₃₁, —C(O)—R₀₁₀, —O—C(O)—R₀₁₁, —C(O)—NR₀₁₁R₀₁₁′, —NR₀₁₁—C(O)—R₀₁₁′, —NR₀₁₁—C(O)—OR₀₁₁′, —(C₁-C₆)alkyl-NR₀₁₁—C(O)—R₀₁₁′, —SO₂—NR₀₁₁ R₀₁₁′, or —SO₂—(C₁-C₆)alkyl,
or the pair (R₀₂, R₀₃) or (R₀₃, R₀₄) together with the carbon atoms to which they are attached form an aromatic or non-aromatic ring containing 5 to 7 ring members, which optionally contains 1 to 3 heteroatoms selected from O, S and N, wherein the ring is optionally substituted by a group selected from a linear or branched (C₁-C₆)alkyl, —NR₀₁₃R₀₁₃′, —(C₀-C₆)alkyl-Cy₀₁ and oxo,
R₀₆ and R₀₇ independently of one another are a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl, a hydroxy group, a linear or branched (C₁-C₆)alkoxy group, a —S—(C₁-C₆)alkyl group, a cyano group, a nitro group, —(C₀-C₆)alkyl-NR₀₁ R₀₁₁′, —O—Cy₀₁, —(C₀-C₆)alkyl-Cy₀₁, —(C₂-C₆)alkenyl-Cy₀₁, —(C₂-C₆)alkynyl-Cy₀₁, —O—(C₁-C₆)alkyl-R₀₁₂, —C(O)—R₀₁₀, —O—C(O)—R₀₁₁, —C(O)—NR₀₁₁ R₀₁₁′, —NR₀₁₁—C(O)—R₀₁₁′, —NR₀₁₁—C(O)—OR₀₁₁′, —(C₁-C₆)alkyl-NR₀₁₁—C(O)—R₀₁₁′, —SO₂—NR₀₁₁R₀₁₁′, or —SO₂—(C₁-C₆)alkyl,
or the pair (R₀₆, R₀₇), when fused with two adjacent carbon atoms, together with the carbon atoms to which they are attached form an aromatic or non-aromatic ring containing 5 to 7 ring members, which optionally contains 1 to 3 heteroatoms selected from O, S and N, and wherein the resulting ring is optionally substituted by a group selected from a linear or branched (C₁-C₆)alkyl group, —NR₀₁₃R₀₁₃′, —(C₀-C₆)alkyl-Cy₀₁ and an oxo,
R₀₈ is a hydrogen atom, a linear or branched (C₁-C₅)alkyl group, an aryl group, a heteroaryl group, an aryl-(C₁-C₆)alkylgroup, or a heteroaryl(C₁-C₆)alkyl group,
R₀₉ is a linear or branched (C₁-C₆)alkyl group, a linear or branched (C₂-C₆)alkenyl group, a linear or branched (C₂-C₆)alkynyl group, —Cy₀₂, —(C₁-C₆)alkyl-Cy₀₂, —(C₂-C₆)alkenyl-Cy₀₂, —(C₂-C₆)alkynyl-Cy₀₂, —Cy₀₂—Cy₀₃, —(C₂-C₆)alkynyl-O—Cy₀₂, —Cy₀₂—(C₀-C₆)alkyl-O—(C₀-C₆)alkyl-Cy₀₃, a halogen atom, a cyano group, —C(O)—R₀₁₄, —C(O)—NR₀₁₄R₀₁₄′,
R₀₁₁ and R₀₁₁′ independently of one another are a hydrogen atom, an optionally substituted linear or branched (C₁-C₆)alkyl group, or —(C₀-C₆)alkyl-Cy₀₁, or the pair (R₀₁₁, R₀₁₁′) together with the nitrogen atom to which they are attached form an aromatic or non-aromatic ring containing 5 to 7 ring members, which optionally contains, in addition to the nitrogen atom, 1 to 3 heteroatoms selected from O, S and N, wherein the N atom is optionally substituted by a linear or branched (C₁-C₆)alkyl group, and wherein one or more of the carbon atoms of the linear or branched (C₁-C₆)alkyl group is optionally deuterated,
R₀₁₂ is —Cy₀₅, —Cy₀₅—(C₀-C₆)alkyl-Cy₀₆, —Cy₀₅—(C₀-C₆)alkyl-O—(C₀-C₆)alkyl-Cy₀₆, —Cy₀₅—(C₀-C₆)alkyl-NR₀₁₁—(C₀-C₆)alkyl-Cy₀₆, —Cy₀₅—Cy₀₆—O—(C₀-C₆)alkyl-Cy₀₇, —Cy₀₅—(C₀-C₆)alkyl-Cy₀₉, —NH—C(O)—NH—R₀₁₁, —C(O)—NR₀₁₁R₀₁₁′, —NR₀₁₁ R₀₁₁′, —OR₀₁₁, —NR₀₁₁—C(O)—R₀₁₁′, —O—(C₁-C₆)alkyl-OR₀₁₁, —SO₂—R₀₁₁, or —C(O)—OR₀₁₁,
R₀₁₃, R₀₁₃′, R₀₁₄ and R₀₁₄′ independently of one another are a hydrogen atom, or an optionally substituted linear or branched (C₁-C₆)alkyl group,
Cy₀₁, Cy₀₂, Cy₀₃, Cy₀₅, Cy₀₆, Cy₀₇ and Cy₀₈ independently of one another, are a cycloalkyl group, a heterocycloalkyl group, an aryl group or a heteroaryl group, each of which is optionally substituted,
Cy₀₉ is

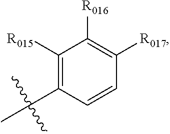

wherein R₀₁₅, R₀₁₆, and R₀₁₇ are as defined for formula (I),
R₀₃₁ is

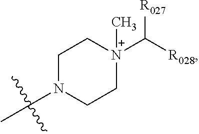

wherein R₀₂₇ and R₀₂₈ are as defined for formula (I)
wherein, at most, one of the R₀₃, R₀₉, or R₀₁₂ groups, if present, is covalently attached to the linker,
or an enantiomer, diastereoisomer, atropisomer, deuterated derivative, and/or pharmaceutically acceptable salt of any of the foregoing.

The definitions of the remaining variables are provided in the fifty-first embodiment or any embodiments described therein.

In a fifty-fourth embodiment, the present disclosure provides an antibody-drug conjugate of the fifty-first embodiment, wherein the Mcl-1 inhibitor is represented by Formula (IB):

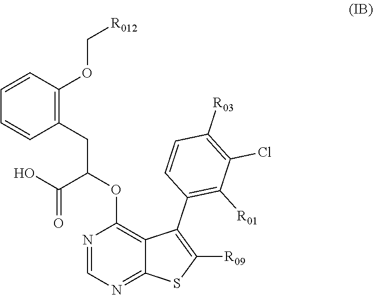

wherein:
R₀₁ is a linear or branched (C₁-C₆)alkyl group,
R₀₃ is —O—(C₁-C₆)alkyl-NR₀₁₁R₀₁₁′, or

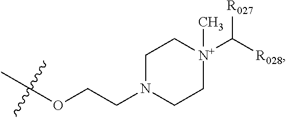

wherein R₀₁₁ and R₀₁₁′ independently of one another are a hydrogen atom, an optionally substituted linear or branched (C₁-C₆)alkyl group, or —(C₀-C₆)alkyl-Cy₀₁;
or the pair (R₀₁₁, R₀₁₁′) together with the nitrogen atom to which they are attached form an aromatic or non-aromatic ring containing 5 to 7 ring members, which optionally contains, in addition to the nitrogen atom, 1 to 3 heteroatoms selected from O, S and N, wherein the N atom may be substituted by 1 or 2 groups selected from a hydrogen atom or a linear or branched (C₁-C₆)alkyl group,
and wherein R₀₂₇ is a hydrogen atom and R₀₂₈ is a —(CH₂)ₚ₀—O—SO₂—O group or a —(CH₂)ₚ₀—SO₂—OR₀₃₀ group;
R₀₉ is a linear or branched (C₂-C₆)alkynyl group or —Cy₀₂,
R₀₁₂ is —Cy₀₈, —Cy₀₅—(C₀-C₆)alkyl-Cy₀₆, or —Cy₀₅—(C₀-C₆)alkyl-Cy₀₉,
Cy₀₁, Cy₀₂, Cy₀₈ and Cy₀₆ independently of one another, are a cycloalkyl group, a heterocycloalkyl group, an aryl group or a heteroaryl group, each of which is optionally substituted,
Cy₀₉ is

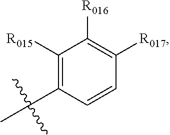

R₀₁₅, R₀₁₆, and Roly are as defined for formula (I),
wherein, at most, one of the R₀₃, R₀₉, or R₀₁₂ groups, if present, is covalently attached to the linker,
or the enantiomer, diastereoisomer, atropisomer, deuterated derivative, and/or pharmaceutically acceptable salt of any of the foregoing.

The definitions of the remaining variables are provided in the fifty-first embodiment.

In a fifty-fifth embodiment, the present disclosure provides an antibody-drug conjugate of the fifty-fourth embodiment, wherein R₀₁ is methyl or ethyl. The definitions of the remaining variables are provided in the fifty-fourth embodiment or any embodiments described therein.

In a fifty-sixth embodiment, the present disclosure provides an antibody-drug conjugate of the fifty-fourth embodiment, wherein R₀₃ is —O—CH₂—CH₂—NR₀₁₁R₀₁₁′ in which R₀ₙ and R₀₁₁′ form, together with the nitrogen atom carrying them, a piperazinyl group which may be substituted by a group being a hydrogen atom or a linear or branched (C₁-C₆)alkyl group). The definitions of the remaining variables are provided in the fifty-fourth embodiment or any embodiments described therein.

In a fifty-seventh embodiment, the present disclosure provides an antibody-drug conjugate of the fifty-fourth embodiment, wherein R₀₃ comprises the formula:

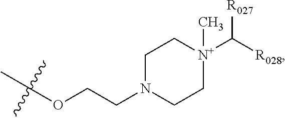

wherein R₀₂₇ is a hydrogen atom and R₀₂₈ is a —(CH₂)ₚ₀—SO₂—OR₀₃₀ group. The definitions of the remaining variables are provided in the fifty-fourth embodiment or any embodiments described therein.

In a fifty-eighth embodiment, the present disclosure provides an antibody-drug conjugate of the fifty-fourth embodiment, wherein R₀₃ comprises the formula:

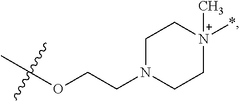

wherein [symbol] is a bond to the linker. The definitions of the remaining variables are provided in the fifty-fourth embodiment or any embodiments described therein.

In a fifty-ninth embodiment, the present disclosure provides an antibody-drug conjugate of the fifty-fourth embodiment, wherein R₀₉ is Cy₀₂. The definitions of the remaining variables are provided in the fifty-fourth embodiment or any embodiments described therein.

In a sixtieth embodiment, the present disclosure provides an antibody-drug conjugate of the fifty-ninth embodiment, wherein Cy₀₂ is an optionally substituted aryl group. The definitions of the remaining variables are provided in the fifty-ninth embodiment or any embodiments described therein.

In a sixty-first embodiment, the present disclosure provides an antibody-drug conjugate of the fifty-fourth embodiment, wherein Cy₀₈ comprises a heteroaryl group selected from a pyrazolyl group and a pyrimidinyl group. The definitions of the remaining variables are provided in the fifty-fourth embodiment or any embodiments described therein.

In a sixty-second embodiment, the present disclosure provides an antibody-drug conjugate of the fifty-fourth embodiment, wherein Cy₀₈ is a pyrimidinyl group. The definitions of the remaining variables are provided in the fifty-fourth embodiment or any embodiments described therein.

In a sixty-third embodiment, the present disclosure provides an antibody-drug conjugate of any one of the fifty-fourth through sixty-second embodiments, wherein the Mcl-1 inhibitor is attached by a covalent bond to R₀₃ of formula (I), (IA), or (IB); or is attached by a covalent bond to R₀₉ of formula (I), (IA), or (IB). The definitions of the remaining variables are provided in the fifty-fourth through sixty-second embodiments or any embodiments described therein.

In a sixty-fourth embodiment, the present disclosure provides an antibody-drug conjugate of any one of the fifty-fourth through sixty-third embodiments, wherein the Mcl-1 inhibitor is represented by any one of the following formulas:

TABLE A1

| | |
|---|---|
| 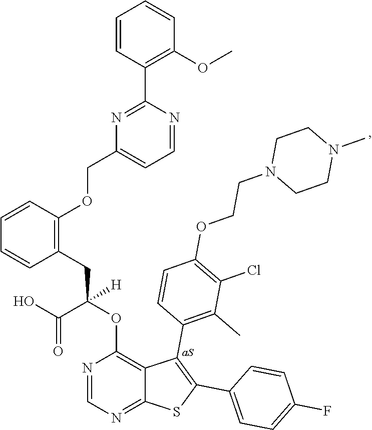 [SMILES: COc1ccccc1-c1nccc(COc2ccccc2C[C@@H](Oc2ncnc3sc(-c4ccc(F)cc4)c(-c4ccc(OCCN5CCN(C)CC5)c(Cl)c4C)c23)C(=O)O)n1] | (D1-1) |
| 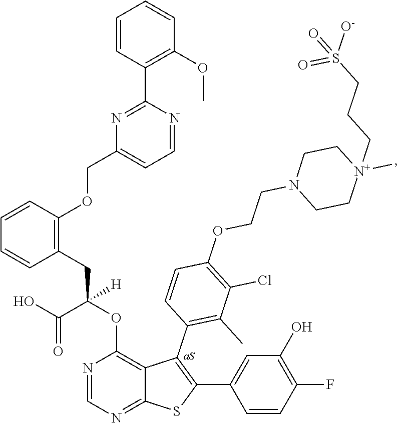 [SMILES: COc1ccccc1-c1nccc(COc2ccccc2C[C@@H](Oc2ncnc3sc(-c4ccc(F)c(O)c4)c(-c4ccc(OCCN5CC[N+](C)(CCCS(=O)(=O)[O-])CC5)c(Cl)c4C)c23)C(=O)O)n1] | (D1-2) |
| 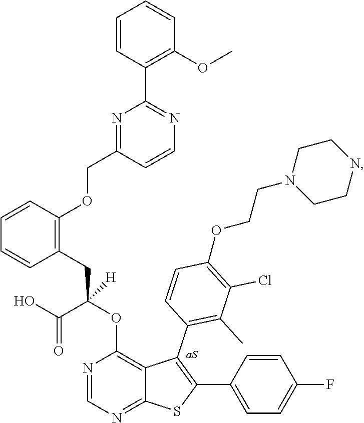 | (D1-3) |
| 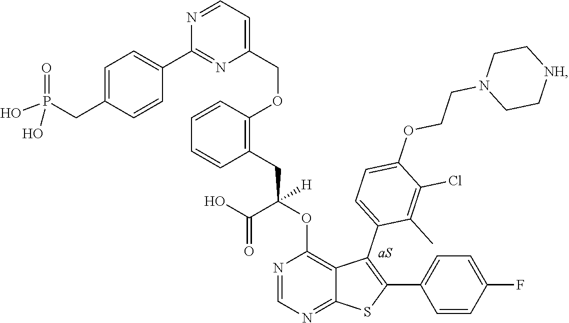 [SMILES: Cc1c(-c2c(-c3ccc(F)cc3)sc3ncnc(O[C@H](Cc4ccccc4OCc4ccnc(-c5ccc(CP(=O)(O)O)cc5)n4)C(=O)O)c23)ccc(OCCN2CCNCC2)c1Cl] | (D1-4) |
| 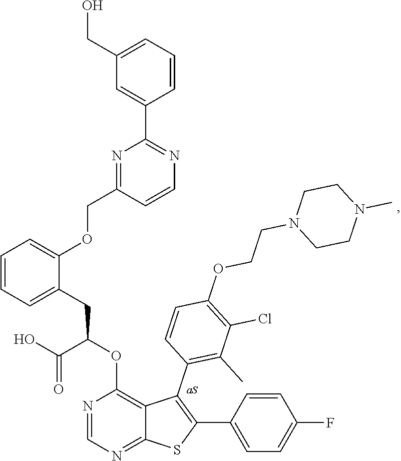 [SMILES: Cc1c(-c2c(-c3ccc(F)cc3)sc3ncnc(O[C@H](Cc4ccccc4OCc4ccnc(-c5cccc(CO)c5)n4)C(=O)O)c23)ccc(OCCN2CCN(C)CC2)c1Cl] | (D1-5) |
| 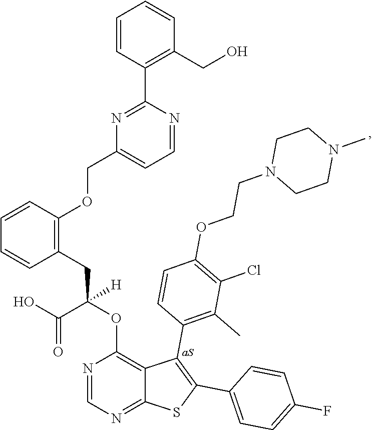 [SMILES: Cc1c(-c2c(-c3ccc(F)cc3)sc3ncnc(O[C@H](Cc4ccccc4OCc4ccnc(-c5ccccc5CO)n4)C(=O)O)c23)ccc(OCCN2CCN(C)CC2)c1Cl] | (D1-6) |
| 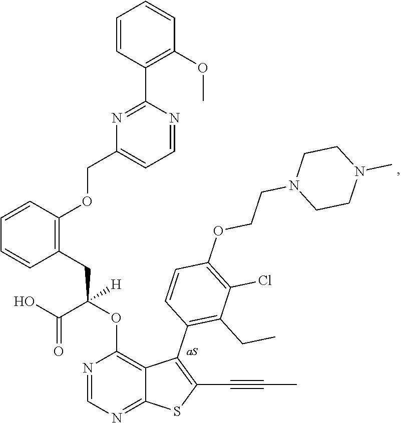 [SMILES: CC#Cc1sc2ncnc(O[C@H](Cc3ccccc3OCc3ccnc(-c4ccccc4OC)n3)C(=O)O)c2c1-c1ccc(OCCN2CCN(C)CC2)c(Cl)c1CC] | (D1-7) |
| 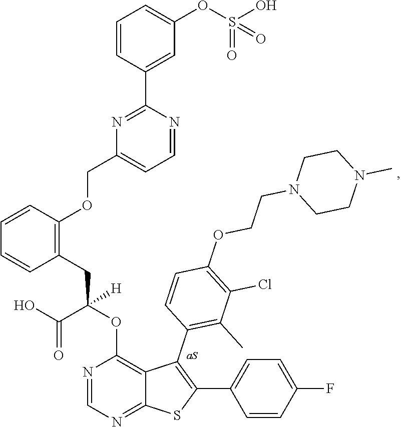 [SMILES: Cc1c(-c2c(-c3ccc(F)cc3)sc3ncnc(O[C@H](Cc4ccccc4OCc4ccnc(-c5cccc(OS(=O)(=O)O)c5)n4)C(=O)O)c23)ccc(OCCN2CCN(C)CC2)c1Cl] | (D1-8) |
| 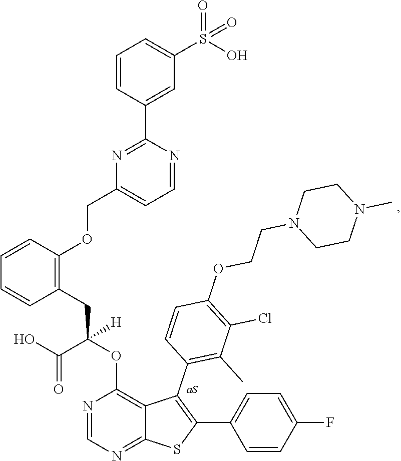 [SMILES: Cc1c(-c2c(-c3ccc(F)cc3)sc3ncnc(O[C@H](Cc4ccccc4OCc4ccnc(-c5cccc(S(=O)(=O)O)c5)n4)C(=O)O)c23)ccc(OCCN2CCN(C)CC2)c1Cl] | (D1-9) |
| 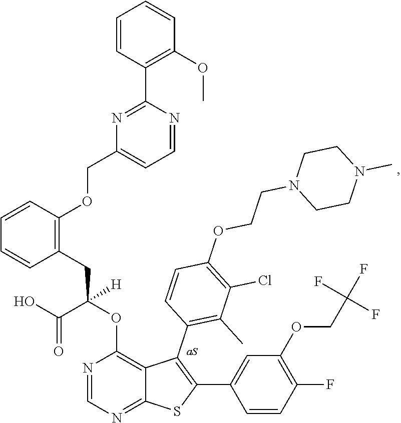 [SMILES: COc1ccccc1-c1nccc(COc2ccccc2C[C@@H](Oc2ncnc3sc(-c4ccc(F)c(OCC(F)(F)F)c4)c(-c4ccc(OCCN5CCN(C)CC5)c(Cl)c4C)c23)C(=O)O)n1] | (D1-10) |
| 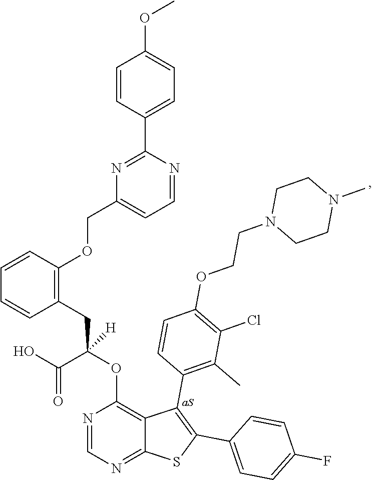 [SMILES: COc1ccc(-c2nccc(COc3ccccc3C[C@@H](Oc3ncnc4sc(-c5ccc(F)cc5)c(-c5ccc(OCCN6CCN(C)CC6)c(Cl)c5C)c34)C(=O)O)n2)cc1] | (D1-11) |
| 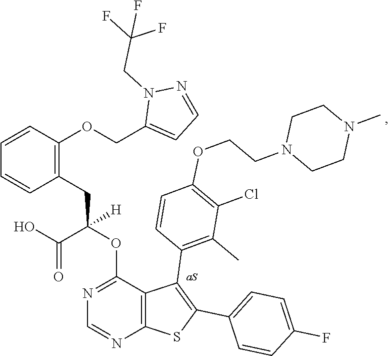 [SMILES: Cc1c(-c2c(-c3ccc(F)cc3)sc3ncnc(O[C@H](Cc4ccccc4OCc4ccnn4CC(F)(F)F)C(=O)O)c23)ccc(OCCN2CCN(C)CC2)c1Cl] | (D1-12) |
| 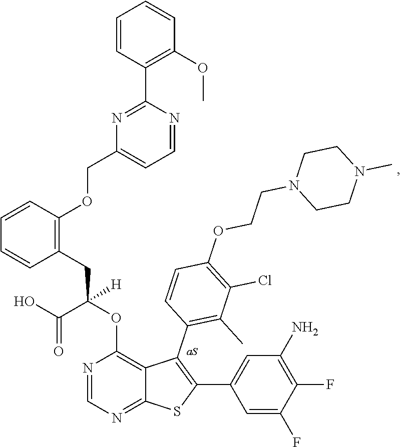 [SMILES: COc1ccccc1-c1nccc(COc2ccccc2C[C@@H](Oc2ncnc3sc(-c4cc(N)c(F)c(F)c4)c(-c4ccc(OCCN5CCN(C)CC5)c(Cl)c4C)c23)C(=O)O)n1] | (D1-13) |
| 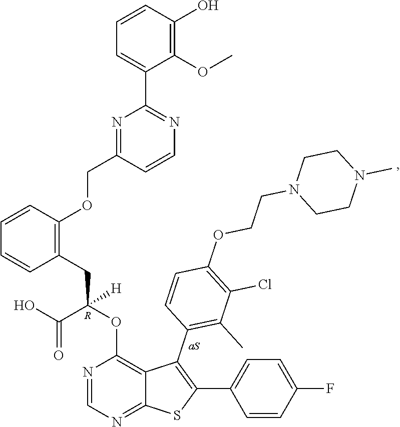 | (D1-14) |
| 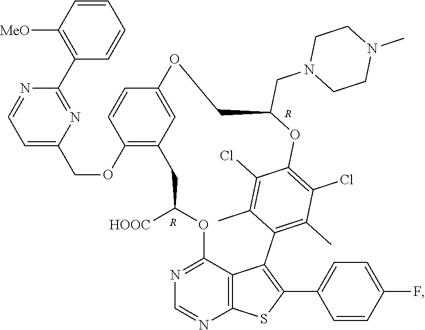 | (D1-15) |
| 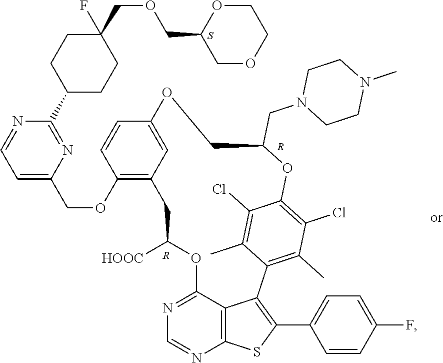 | (D1-16) |
| [symbol] | (D1-17) |

[symbol]

indicates data missing or illegible when filed

or an enantiomer, diastereoisomer, atropisomer, deuterated derivative, and/or pharmaceutically acceptable salt of any of the foregoing. The definitions of the remaining variables are provided in the fifty-fourth through sixty-third embodiments or any embodiments described therein.

In a sixty-fifth embodiment, the present disclosure provides an antibody-drug conjugate of any one of the forty-sixth through fiftieth embodiments, wherein the Bcl-xL inhibitor is represented by Formula (II) or Formula (III):

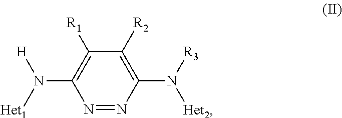

or an enantiomer, a diastereoisomer, and/or a pharmaceutically acceptable salt of any one of the foregoing, wherein:
R₁ and R₂ independently of one another represent a group selected from the group consisting of: hydrogen; a linear or branched C₁-C₆alkyl optionally substituted by a hydroxyl or a C₁-C₆alkoxy group; a C₃-C₆cycloalkyl; a trifluoromethyl; and a linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group, R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:

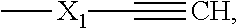

Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O—; a C₁-C₆alkylene-COOH; a C₁-C₆alkylene-PO(OH)₂; a C₁-C₆alkylene-NRₐRₑ; a C₁-C₆alkylene-N⁺RₐRₑR_{f}; a C₁-C₆alkylene-phenyl wherein the phenyl may be substituted by a C₁-C₆alkoxy group; and the group:

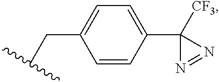 [SMILES: *Cc1ccc(C2(C(F)(F)F)N=N2)cc1]

or Rₐ and R_{b} form with the nitrogen atom carrying them a cycle Bi;
or Rₐ, R_{b} and R_{c} form with the nitrogen atom carrying them a bridged C₃-C₈ hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈ hetero cycloalkyl,
Het₁ represents a group selected from the group consisting of:

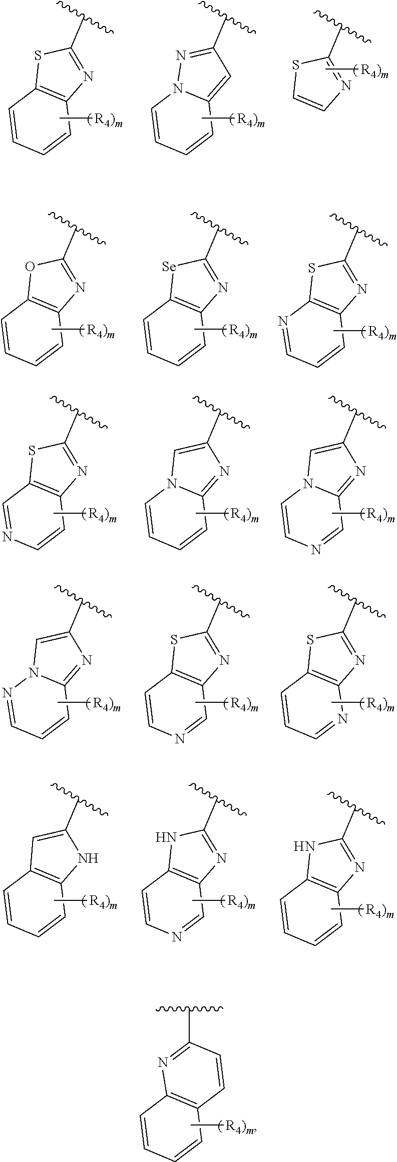

Het₂ represents a group selected from the group consisting of:

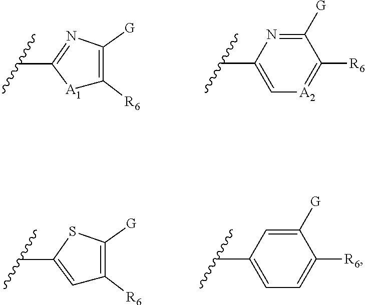

A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2},
—OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, -C₁-C₆alkyl optionally substituted by a hydroxyl group, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₅ heterocycloalkyl; or in the alternative, G is selected from the group consisting of:

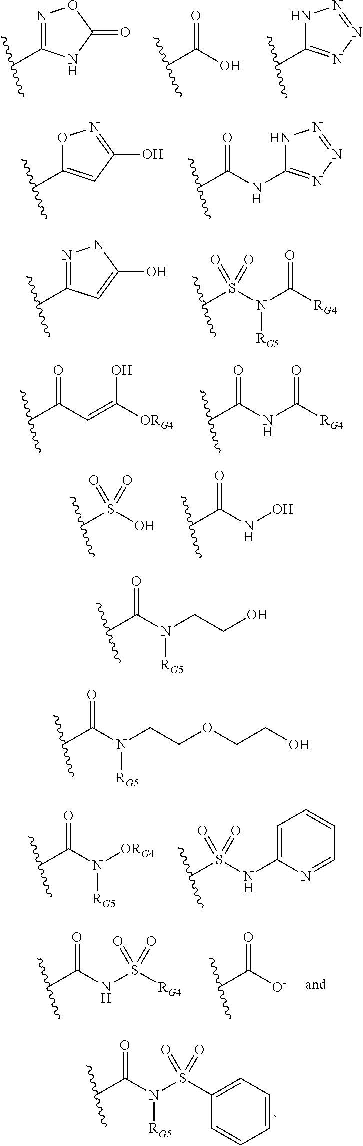

wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched -C₁-C₆alkylene-R₈ group;
a -C₂-C₆alkenyl;
X₂—O-R₇;

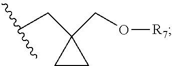

—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂-R₇;
a C₃-C₆heterocycloalkylene-Y₂-R₇ group, and
a heteroarylene-R₇ group optionally substituted by a linear or branched C₁-C₆alkyl group, R₇ represents a group selected from the group consisting of: a linear or branched C₁-C₆alkyl group; a (C₃-C₆)cycloalkylene-R₈;

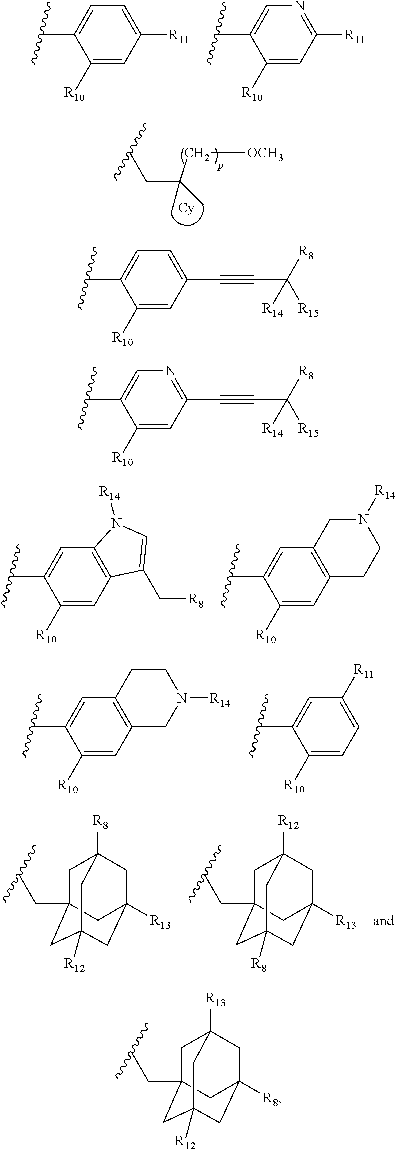

wherein Cy represents a C₃-C₅ cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′; —N⁺R′ₐR′_{b}R′_{c}; —O—R′_{c}; —NH—X′₂—N⁺R′ₐR′_{b}R¹; —O—X′₂—NR′ₐR′_{b}; —X′₂—NR′ₐR′_{b}; —NR′_{c}—X′₂—N₃ and

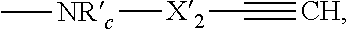

R₉ represents a group selected from the group consisting of a linear or branched C₁-C₆alkyl, trifluoromethyl, hydroxyl, halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, a —O—C₁-C₃alkylene-R₈, —CO-NRₕRᵢ and a —CH═CH-C₁-C₄alkylene-NRRᵢ, —CH═CH—CHO, a C₃-C₈cycloalkylene-CH₂—R₈, and a C₃-C₅ heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and
R₁₅ form with the carbon atom carrying them a cyclohexyl,
Rₕ and Rᵢ, independently of one another, represent a hydrogen or a linear or branched C₁-C₆alkyl group,
X, and X₂ independently of one another, represent a linear or branched C₁-C₆alkylene group optionally substituted by one or two groups selected from the group consisting of trifluoromethyl, hydroxyl, a halogen, and a C₁-C₆alkoxy, X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR′ₐR′ₑ; a C₁-C₆alkylene-N⁺R′ₐR′,R″; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

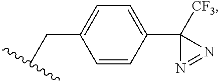 [SMILES: *Cc1ccc(C2(C(F)(F)F)N=N2)cc1]

or R′ₐ and R′_{b} form with the nitrogen atom carrying them a cycle B₃, or R′ₐ, R′_{b} and R′_{c} form with the nitrogen atom carrying them a bridged C₃-C₈ hetero cycloalkyl,
R′_{c}, R¹ , R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₅ heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃ and R₈ groups, if present, is covalently attached to the linker, and wherein the valency of an atom is not exceeded by virtue of one or more substituents bonded thereto; or

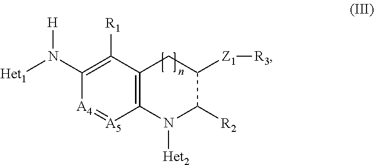

or an enantiomer, a diastereoisomer, and/or a pharmaceutically acceptable salt of any one of the foregoing, wherein:
n=0, 1 or 2,
[symbol] represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆ alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group; R₂represents a hydrogen or a methyl;
R₃ represents a group selected from the group consisting of: hydrogen; a linear or branched C₁-C₄ alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:

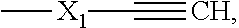

Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O—; a C₁-C₆alkylene-COOH; a C₁-C₆alkylene-PO(OH)₂; ₐC₁-C₆alkylene-NRₐRₑ; a C₁-C₆alkylene-N⁺RₐRₑR_{f}; a C₁-C₆alkylene-phenyl wherein the phenyl may be substituted by a C₁-C₆alkoxy group; and the group:

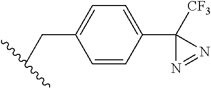 [SMILES: *Cc1ccc(C2(C(F)(F)F)N=N2)cc1]

or Rₐ and R_{b} form with the nitrogen atom carrying them a cycle Bi;
or Rₐ, R_{b} and R_{c} form with the nitrogen atom carrying them a bridged C₃-C₅ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₅ heterocycloalkyl,

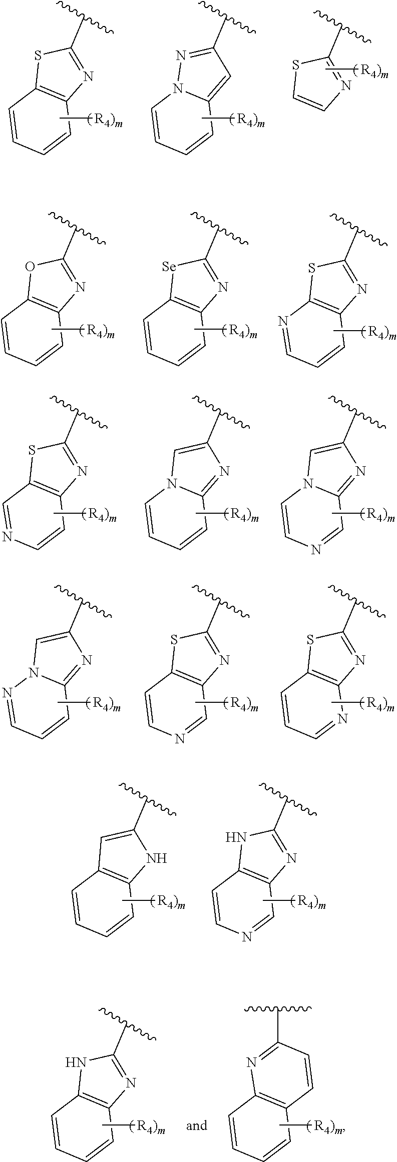

Het₁ represents a group selected from the group consisting of:

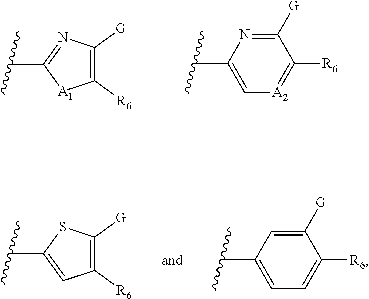

A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, -C₁-C₆alkyl optionally substituted by a hydroxyl group, halogen, —NO₂, and —CN, in which:
R_{G}, and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆ alkynyl, a C₃-C₆cycloalkyl, phenyl and —(CH₂)1_₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)1_₄-phenyl; or R_{GI} and R_{G2}, together with the atom to which each is attached are combined to form a C₃-C₈heterocycloalkyl; or in the alternative, G is selected from the group consisting of:

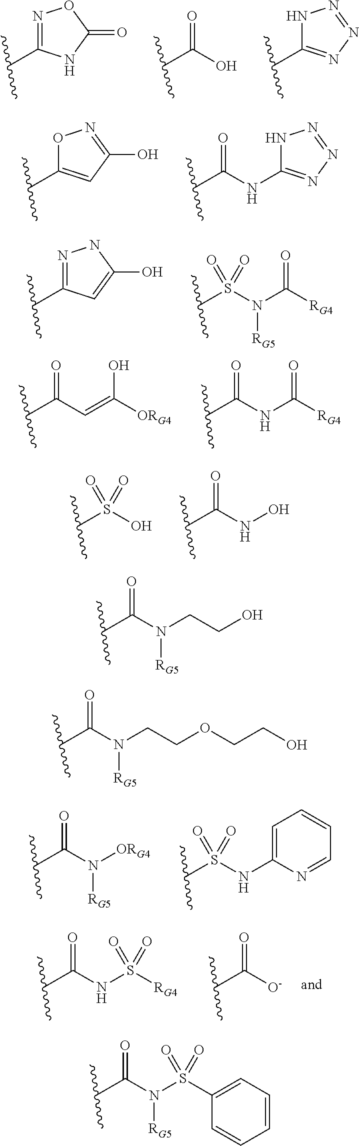

wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆ alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched -C₁-C₆alkylene-R₈ group;
a -C₂-C₆alkenyl;
—X₂—O—R₇;

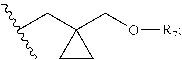

—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂-R₇;
a C₃-C₆heterocycloalkylene-Y₂-R₇ group, and
a heteroarylene-R₇ group optionally substituted by a linear or branched C₁-C₆alkyl group, R₇ represents a group selected from the group consisting of: a linear or branched C₁-C₆alkyl group; a (C₃-C₆)cycloalkylene-R₈;

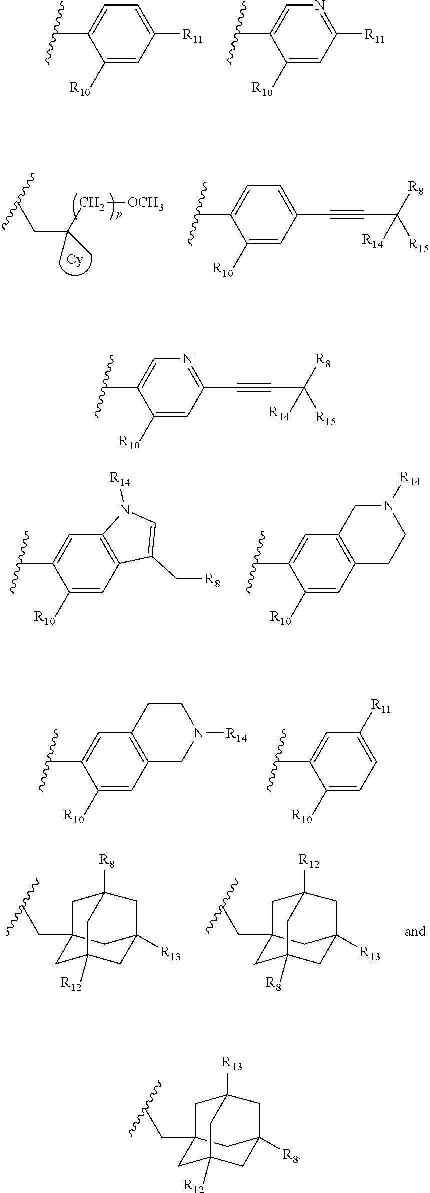

wherein Cy represents a C₃-C₅ cycloalkyl,
R₈ represents a group selected from the group consisting of: hydrogen; a linear or branched C₁-C₆alkyl, —NR′ₐR′_{b}; —NR′ₐ—CO—OR′c; —NR′ₐ—CO—R′; —N⁺R′ₐR′_{b}R′_{c}; —O—R′_{c}; —NH—X′₂—N⁺R′ₐR′_{b}R¹; —O—X′2—NR′ₐR′b, —X′2—NR′ₐR′b, —NR′_{c}—X′₂—N₃ and:

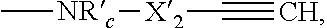

R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₅ cycloalkylene-CH₂—R₈, and a C₃-C₅ heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR′ₐR′ a C₁-C₆alkylene-N⁺R′ₐR′,R″; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

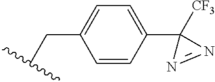 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—, m=0, 1 or 2,
B₁, B₂, B₃ and B₄, independently of one another, represents a C₃-C₅ heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,

wherein one of the R₃, R₈ and G groups, if present, is covalently attached to the linker, and

wherein the valency of an atom is not exceeded by virtue of one or more substituents bonded thereto. The definitions of the remaining variables are provided in the forty-sixth through fiftieth embodiments or any embodiments described therein.

In a sixty-sixth embodiment, the present disclosure provides an antibody-drug conjugate of the sixty-fifth embodiment, wherein the Bcl-xL inhibitor is represented by formula (IIA) or (IIIA):

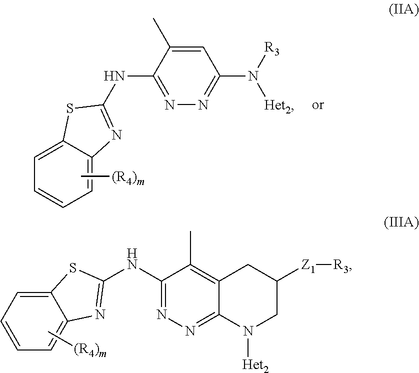

or an enantiomer, a diastereoisomer, and/or a pharmaceutically acceptable salt of any one of the foregoing, wherein:
Z, represents a bond or —O—,
R₃ represents a group selected from the group consisting of: hydrogen; a C₃-C₆cycloalkyl; a linear or branched C₁-C₆alkyl; —X₁—NRₐR_{b}; —X₁—N′RₐR_{b}R_{c}; —X₁—O—R_{c}; —X₁—N₃ and

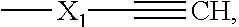

Rₐ and R_{b} independently of one another represent a group selected from the group consisting of: hydrogen; a linear or branched C₁-C₆alkyl optionally substituted by one or two hydroxyl groups; and a C₁-C₆alkylene-SO₂O—,
R_{c} represents a hydrogen or a linear or branched C₁-C₆alkyl group,
Het₂ represents a group selected from the group consisting of:

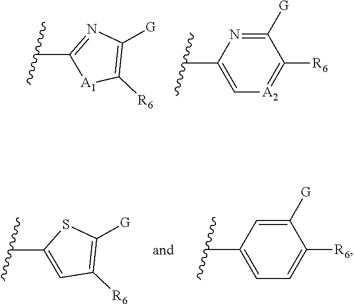

A₁ is —NH—, —N(C₁-C₃alkyl), O, S or Se,
A₂ is N, CH or C(R₅),
G is selected from the group consisting of:
—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, -C₁-C₆alkyl optionally substituted by a hydroxyl group, —C(O)NR_{G5}S(O)₂R_{G4}, halogen, —NO₂, and —CN, in which:
R_{G1}, R_{G2}, R_{G4} and R_{G5} at each occurrence are each independently selected from the group consisting of hydrogen, and a C₁-C₆alkyl optionally substituted by 1 to 3 halogen atoms;
R_{G3} is a C₁-C₆alkyl optionally substituted by 1 to 3 halogen atoms; or R_{GI} and R_{G2}, together with the atom to which each is attached are combined to form a C₃-C₈heterocycloalkyl;
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a halogen and —CN, R₆ represents a group selected from the group consisting of:
a linear or branched -C₁-C₆alkylene-R₈ group; —X₂—O—R₇; and
a heteroarylene-R₇ group optionally substituted by a linear or branched C₁-C₆alkyl group, R₇ represents a group selected from the group consisting of: a linear or branched C₁-C₆alkyl group; (C₃-C₆)cycloalkylene-R₈;

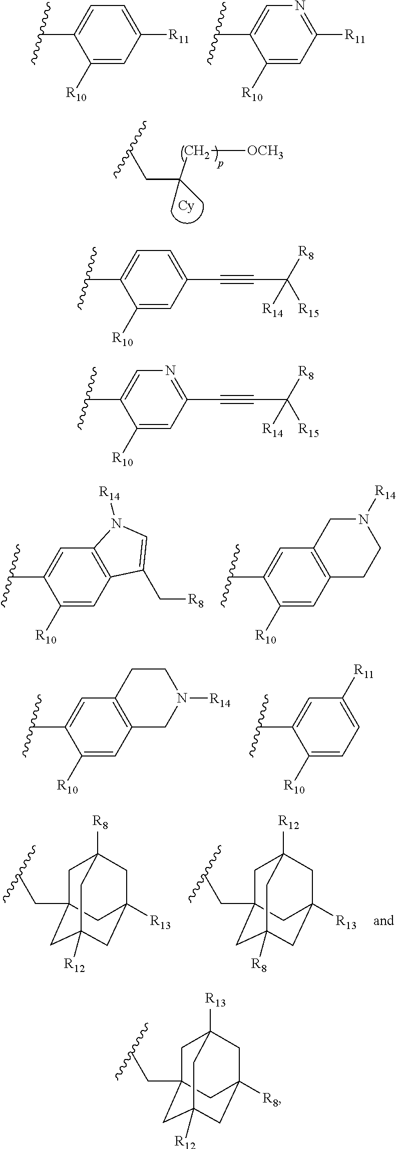

Rs represents a group selected from the group consisting of: hydrogen, a Cina-C₃alklnceR,d OC-Calkylen- -C-NR′ₐ‘b-N’aC-Rᵢ, —CH═CH—C-₄akyen-NRaRi,_{b}R -H═C -H-CHO a₂C-Cscyclalkylen-CH2NR R X 2R₈,aR and aN—X2N C₃Chtrcylaklne-H₂Rs

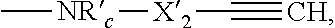

R₁—NdR₁₃,X inepnenl ofGoe-anth,C rpeetahdrgnao raehlgop R₁₄an repesns, indeopendet ofoe anther greuprensen f hydrogen, ramehlurop, horiR₄nd, Ris foprmswith th cron aeetom carryin them au ossigo yrgn _{I}Cakln-s ascyclohexyl,n-H-s n ₃Chteoylakln-H-s Rₕ and Rᵢ, independently of one another, represent a hydrogen or a linear or branched Ci-CGalkyl group,
X, and X₂ independently of one another, represent a linear or branched
C₁-C₆alkylene group optionally substituted by one or two groups selected from the group consisting of trifluoromethyl, hydroxyl, a halogen, and C₁-C₆alkoxy, X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR′ₐR′ₑ a C₁-C₆alkylene-N⁺R′ₐR′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: CF3

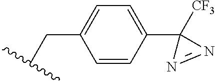 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′, form with the nitrogen atom carrying them a bridged C₃-C₅ heterocycloalkyl,
R′_{c}, R′, R′ₑ, R′_{f}, independently of one another, represents a hydrogen or a linear or branched C₁-C₆alkyl group,
or R′_{d} and R′ₑ form with the nitrogen atom carrying them a cycle B₄, or R′_{d}, R′ₑ and R′_{f} form with the nitrogen atom carrying them a bridged C₃-C₈heterocycloalkyl,
m=0, 1 or 2,
p=1, 2, 3 or 4,
B₃ and B₄, independently of one another, represents a C₃-C₅ heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl.

The definitions of the remaining variables are provided in the sixty-fifth embodiment or any embodiments described therein.

In a sixty-seventh embodiment, the present disclosure provides an antibody-drug conjugate of the sixty-sixth embodiment, wherein G is selected from the group consisting of: —C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN. The definitions of the remaining variables are provided in the sixty-sixth embodiment or any embodiments described therein.

In a sixty-eighth embodiment, the present disclosure provides an antibody-drug conjugate of any one of the sixty-fifth through sixty-seventh embodiments, wherein R₇ represents a group selected from the group consisting of: a linear or branched C₁-C₆alkyl group; a (C₃—C)cycloalkylene-R₈;

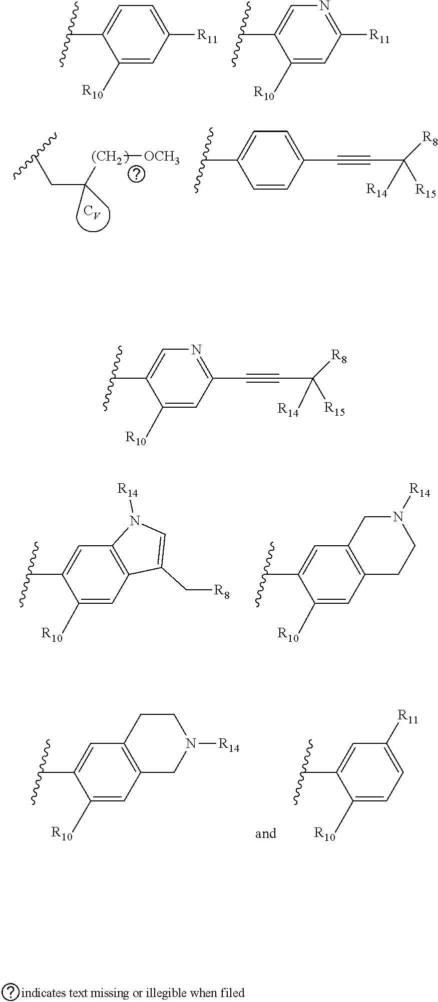

wherein Cy represents a C₃-C₈cycloalkyl. The definitions of the remaining variables are provided in the sixty-fifth through sixty-seventh embodiments or any embodiments described therein.

In a sixty-ninth embodiment, the present disclosure provides an antibody-drug conjugate of any one of the sixty-fifth through sixty-seventh embodiments, wherein R₇ represents a group selected from the group consisting of:

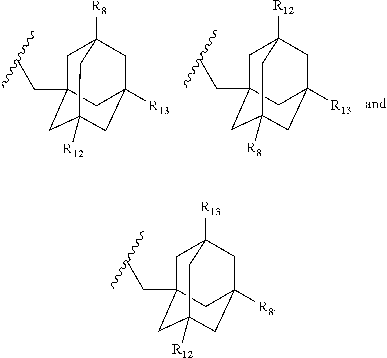

The definitions of the remaining variables are provided in the sixty-fifth through sixty-seventh embodiments or any embodiments described therein.

In a seventieth embodiment, the present disclosure provides an antibody-drug conjugate of the sixty-fifth embodiment, wherein the Bel-xL inhibitor is represented by formula (IIB), (IIC), (IIIB) or

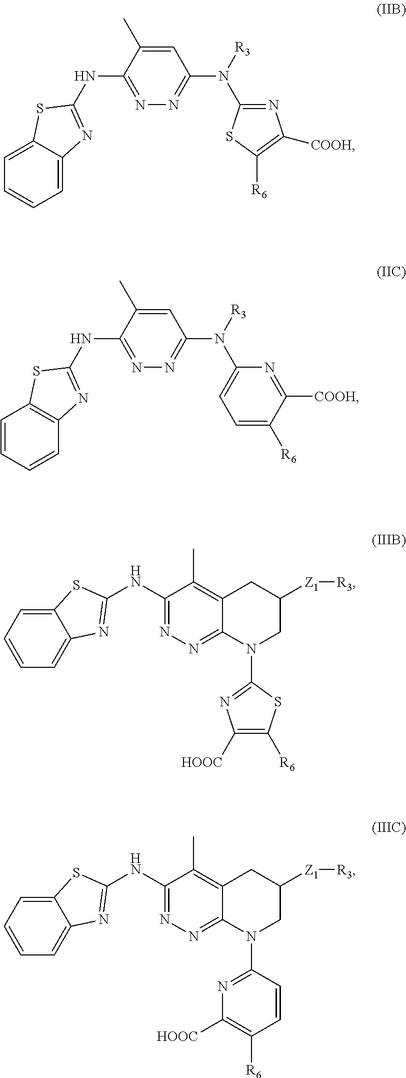

or an enantiomer, a diastereoisomer, and/or a pharmaceutically acceptable salt of any one of the foregoing, wherein: for formula (IIB) or (IIC), R₃ represents a group selected from: hydrogen; linear or branched C₁-C₆alkyl; —X₁—NRₐR_{b}; —X₁—N⁺RₐR_{b}R_{c}; and —X₁—O—R_{c};
for formula (IIIB) or (IIIC), Z₁ represents a bond, and R₃ represents hydrogen; or Z₁ represents —O—, and R₃ represents —X₁—NRₐR_{b},
Rₐ and R_{b} independently of one another represent a group selected from: hydrogen; linear or branched C₁-C₆alkyl optionally substituted by one or two hydroxyl groups; and C₁-C₆alkylene-SO₂O-,
R_{c} represents a hydrogen or a linear or branched C₁-C₆alkyl group
R₆ represents —X₂—O—R₇ or an heteroarylene-R₇ group optionally substituted by a linear or branched C₁-C₆alkyl group,
R₇ represents a group selected from:

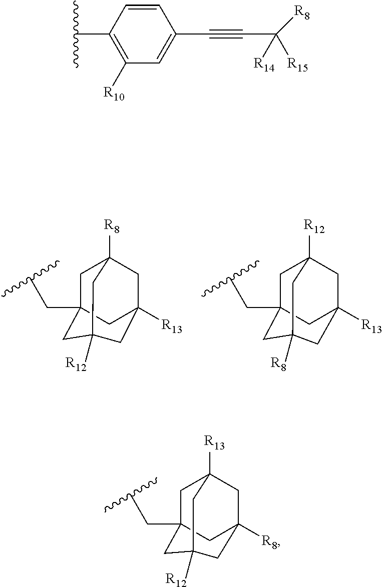

R₈ represents a group selected from: —NR′ₐR′_{b}; —O—X′₂—NR′ₐR′_{b}; and —X′2—NR′ₐR′_{b}, R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂represents a linear or branched C₁-C₆alkylene,
R′ₐ and R′_{b} independently of one another, represent a group selected from: hydrogen; linear or branched C₁-C₆alkyl optionally substituted by one or two hydroxyl or C₁-C₆alkoxy groups; C₁-C₆alkylene-NR′ₐR′ₑ
or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₅ heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from: fluorine, bromine, chlorine, linear or branched C₁-C₆alkyl, hydroxyl, and oxo.

The definitions of the remaining variables are provided in the sixty-fifth embodiment or any embodiments described therein.

In a seventy-first embodiment, the present disclosure provides an antibody-drug conjugate of any one of the sixty-fifth through seventieth embodiments, wherein R₇represents the following group:

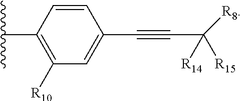

The definitions of the remaining variables are provided in the sixty-fifth through seventieth embodiments or any embodiments described therein.

In a seventy-second embodiment, the present disclosure provides an antibody-drug conjugate of any one of the sixty-fifth through seventieth embodiments, wherein R₇ represents a group selected

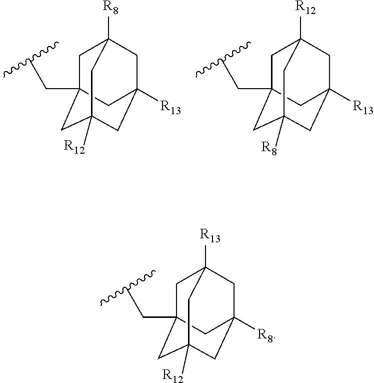

The definitions of the remaining variables are provided in the sixty-fifth through seventieth embodiments or any embodiments described therein.

In a seventy-third embodiment, the present disclosure provides an antibody-drug conjugate of any one of the sixty-fifth through seventy-second embodiments, wherein R₈ represents a group selected from:

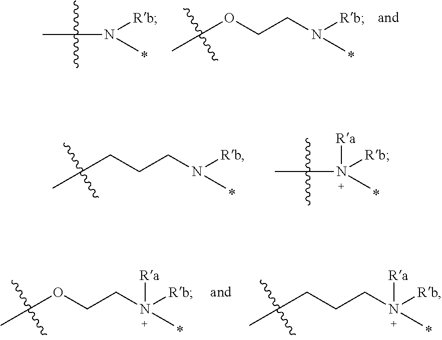

wherein [symbol] represents a bond to the linker.

The definitions of the remaining variables are provided in the sixty-fifth through seventy-second embodiments or any embodiments described therein.

In a seventy-fourth embodiment, the present disclosure provides an antibody-drug conjugate of any one of the sixty-fifth through seventy-third embodiments, wherein B₃ represents a C₃-C₈heterocycloalkyl group selected from a pyrrolidinyl group, a piperidinyl group, a piperazinyl group, a morpholinyl group, an azepanyl group, and a 4,4-difluoropiperidin-1-yl group. The definitions of the remaining variables are provided in the sixty-fifth through seventy-third embodiments or any embodiments described therein.

In a seventy-fifth embodiment, the present disclosure provides an antibody-drug conjugate of the sixty-fifth embodiment, wherein the Bcl-xL inhibitor is represented by any one of the following:

TABLE A2

| | |
|---|---|
| 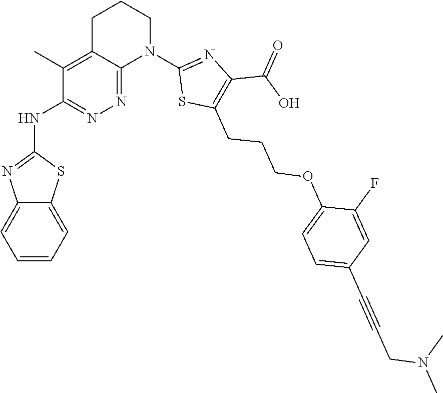 [SMILES: Cc1c(Nc2nc3ccccc3s2)nnc2c1CCCN2c1nc(C(=O)O)c(CCCOc2ccc(C#CCN(C)C)cc2F)s1] | D2-1 |
| 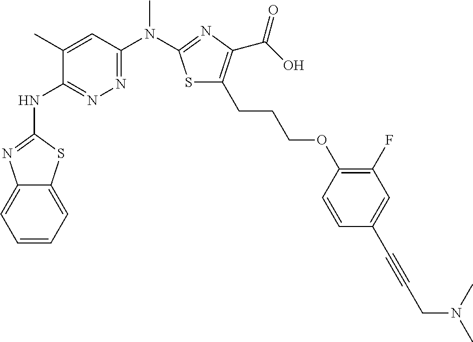 [SMILES: Cc1cc(N(C)c2nc(C(=O)O)c(CCCOc3ccc(C#CCN(C)C)cc3F)s2)nnc1Nc1nc2ccccc2s1] | D2-2 |
| 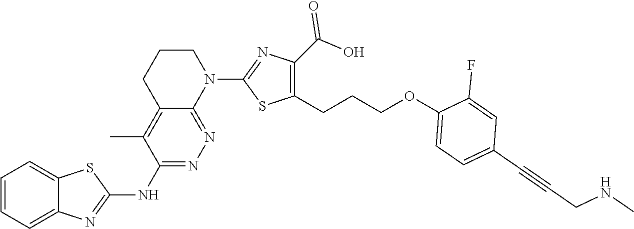 [SMILES: CNCC#Cc1ccc(OCCCc2sc(N3CCCc4c3nnc(Nc3nc5ccccc5s3)c4C)nc2C(=O)O)c(F)c1] | D2-3 |
| 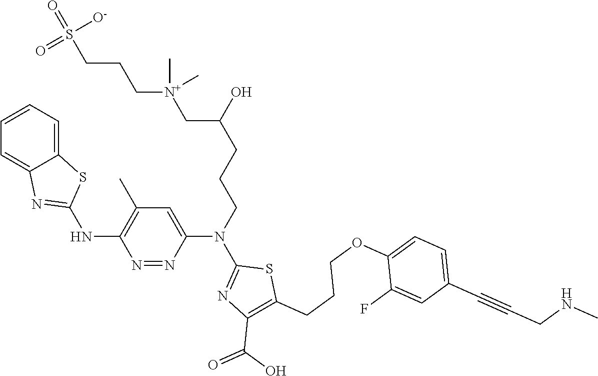 [SMILES: CNCC#Cc1ccc(OCCCc2sc(N(CCCC(O)C[N+](C)(C)CCCS(=O)(=O)[O-])c3cc(C)c(Nc4nc5ccccc5s4)nn3)nc2C(=O)O)c(F)c1] | D2-4 |
| 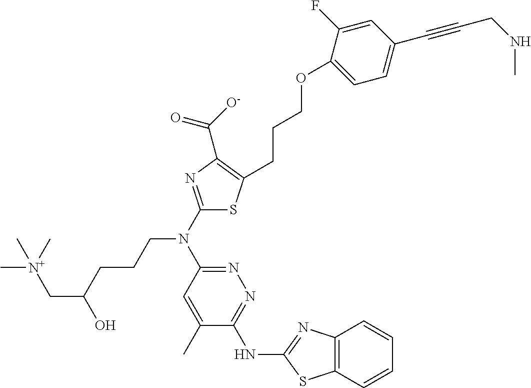 | D2-5 |
| 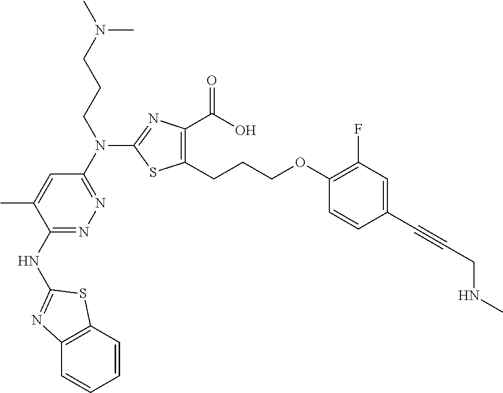 | D2-6 |
| 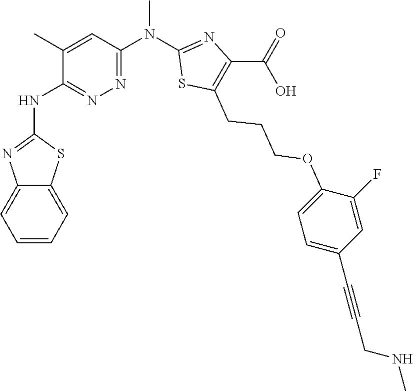 [SMILES: CNCC#Cc1ccc(OCCCc2sc(N(C)c3cc(C)c(Nc4nc5ccccc5s4)nn3)nc2C(=O)O)c(F)c1] | D2-7 |
| 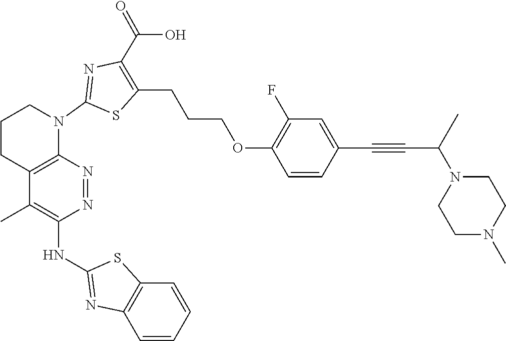 | D2-8 |
| 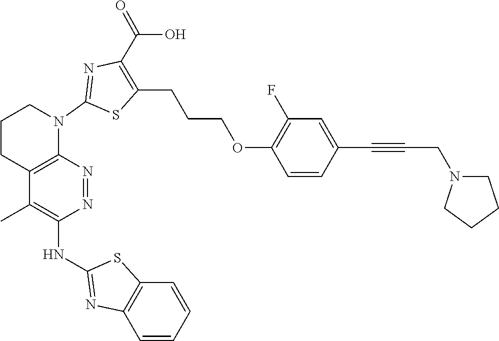 [SMILES: Cc1c(Nc2nc3ccccc3s2)nnc2c1CCCN2c1nc(C(=O)O)c(CCCOc2ccc(C#CCN3CCCC3)cc2F)s1] | D2-9 |
| 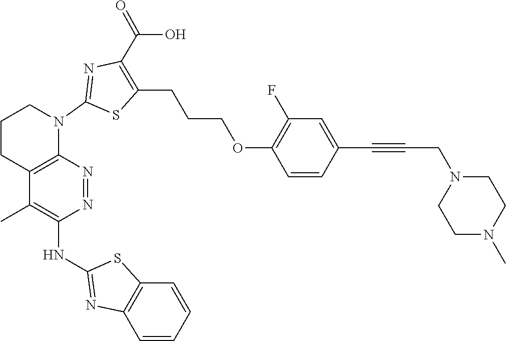 | D2-10 |
| 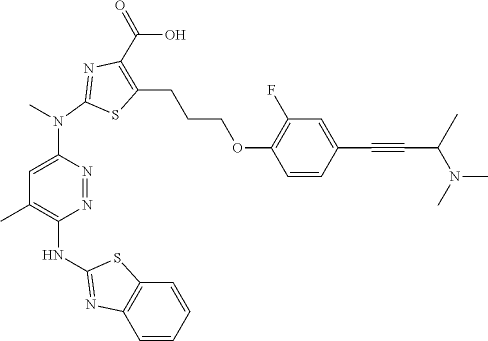 | D2-11 |
| 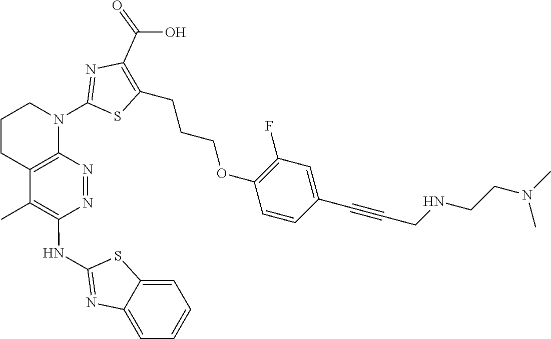 [SMILES: Cc1c(Nc2nc3ccccc3s2)nnc2c1CCCN2c1nc(C(=O)O)c(CCCOc2ccc(C#CCNCCN(C)C)cc2F)s1] | D2-12 |
| 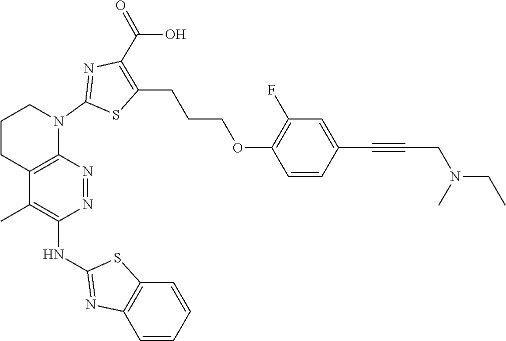 [SMILES: CCN(C)CC#Cc1ccc(OCCCc2sc(N3CCCc4c3nnc(Nc3nc5ccccc5s3)c4C)nc2C(=O)O)c(F)c1] | D2-13 |
| 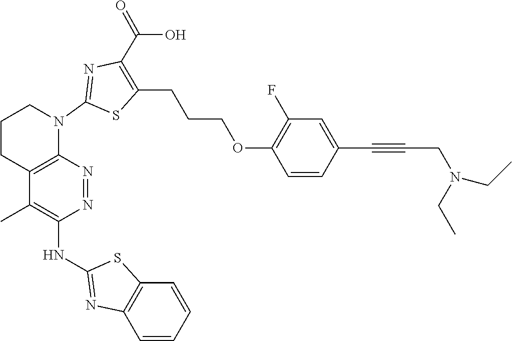 [SMILES: CCN(CC)CC#Cc1ccc(OCCCc2sc(N3CCCc4c3nnc(Nc3nc5ccccc5s3)c4C)nc2C(=O)O)c(F)c1] | D2-14 |
| 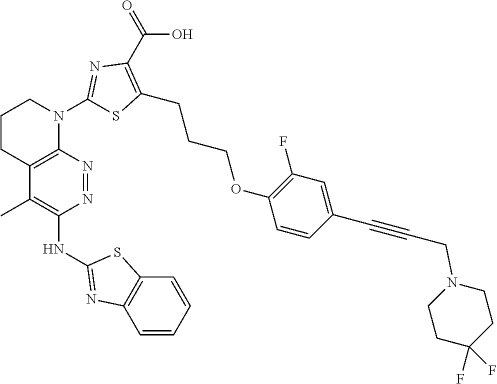 [SMILES: Cc1c(Nc2nc3ccccc3s2)nnc2c1CCCN2c1nc(C(=O)O)c(CCCOc2ccc(C#CCN3CCC(F)(F)CC3)cc2F)s1] | D2-15 |
| 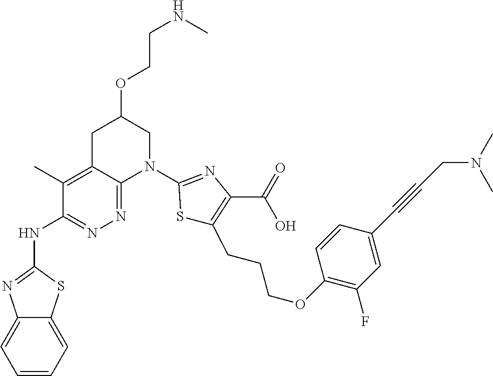 [SMILES: CNCCOC1Cc2c(nnc(Nc3nc4ccccc4s3)c2C)N(c2nc(C(=O)O)c(CCCOc3ccc(C#CCN(C)C)cc3F)s2)C1] | D2-16 |
| 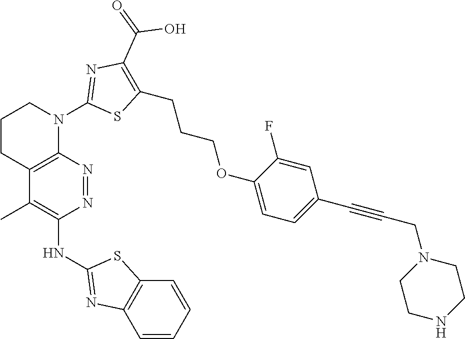 [SMILES: Cc1c(Nc2nc3ccccc3s2)nnc2c1CCCN2c1nc(C(=O)O)c(CCCOc2ccc(C#CCN3CCNCC3)cc2F)s1] | D2-17 |
| 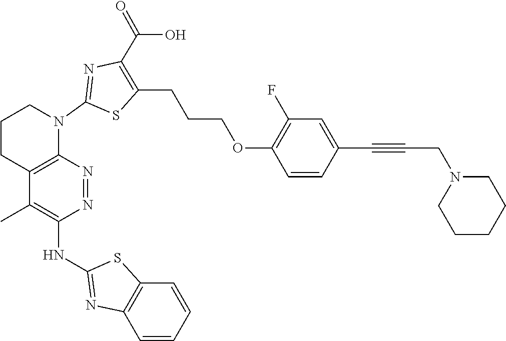 [SMILES: Cc1c(Nc2nc3ccccc3s2)nnc2c1CCCN2c1nc(C(=O)O)c(CCCOc2ccc(C#CCN3CCCCC3)cc2F)s1] | D2-18 |
| 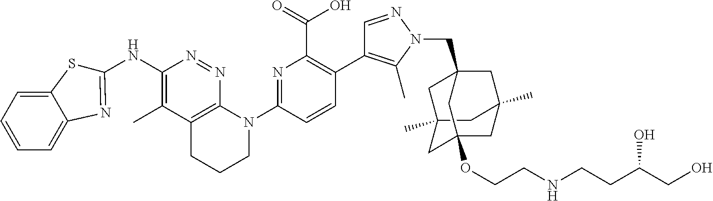 [SMILES: Cc1c(Nc2nc3ccccc3s2)nnc2c1CCCN2c1ccc(-c2cnn(C[C@]34C[C@]5(C)C[C@](C)(C3)C[C@@](OCCNCC[C@H](O)CO)(C5)C4)c2C)c(C(=O)O)n1] | D2-19 |
| 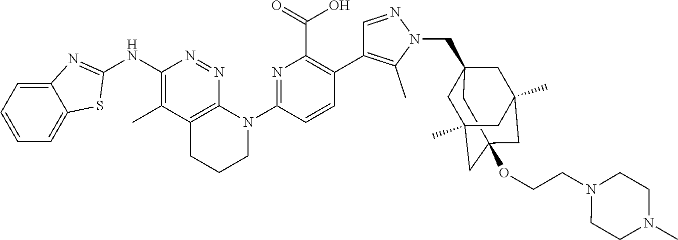 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCN(C)CC3)(C5)C4)c2C)c(C(=O)O)n1] | D2-20 |
| 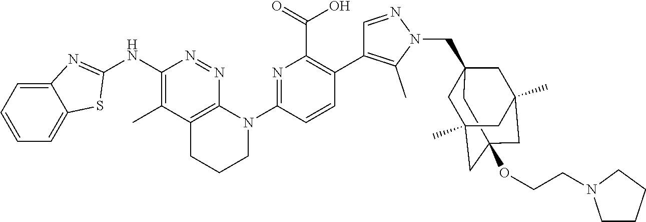 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCCC3)(C5)C4)c2C)c(C(=O)O)n1] | D2-21 |
| 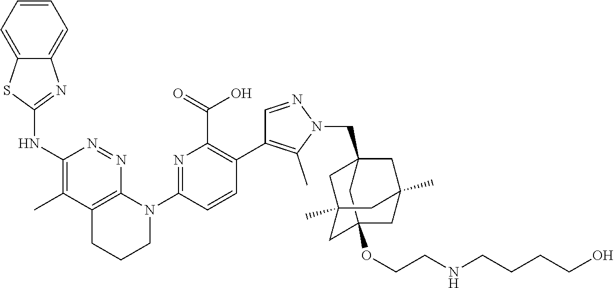 [SMILES: Cc1c(Nc2nc3ccccc3s2)nnc2c1CCCN2c1ccc(-c2cnn(C[C@]34C[C@]5(C)C[C@](C)(C3)C[C@@](OCCNCCCCO)(C5)C4)c2C)c(C(=O)O)n1] | D2-22 |
| 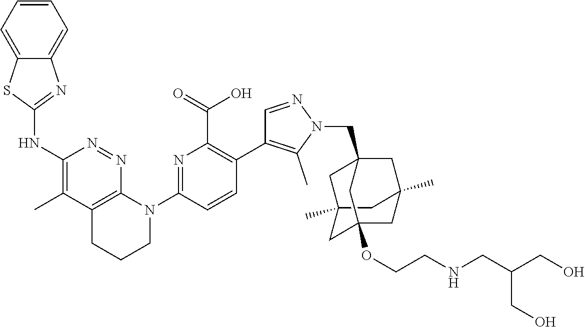 [SMILES: Cc1c(Nc2nc3ccccc3s2)nnc2c1CCCN2c1ccc(-c2cnn(C[C@]34C[C@]5(C)C[C@](C)(C3)C[C@@](OCCNCC(CO)CO)(C5)C4)c2C)c(C(=O)O)n1] | D2-23 |
| 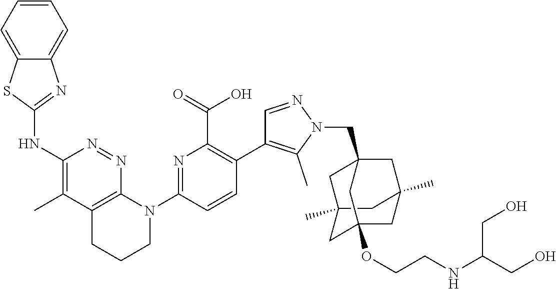 [SMILES: Cc1c(Nc2nc3ccccc3s2)nnc2c1CCCN2c1ccc(-c2cnn(C[C@]34C[C@]5(C)C[C@](C)(C3)C[C@@](OCCNC(CO)CO)(C5)C4)c2C)c(C(=O)O)n1] | D2-24 |
| 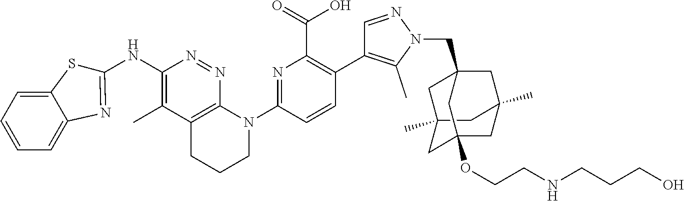 [SMILES: Cc1c(Nc2nc3ccccc3s2)nnc2c1CCCN2c1ccc(-c2cnn(C[C@]34C[C@]5(C)C[C@](C)(C3)C[C@@](OCCNCCCO)(C5)C4)c2C)c(C(=O)O)n1] | D2-25 |
| 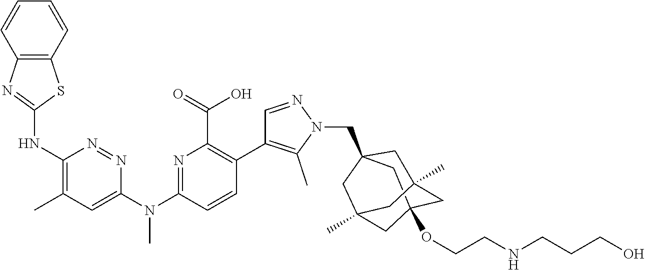 [SMILES: Cc1cc(N(C)c2ccc(-c3cnn(C[C@]45C[C@]6(C)C[C@](C)(C4)C[C@@](OCCNCCCO)(C6)C5)c3C)c(C(=O)O)n2)nnc1Nc1nc2ccccc2s1] | D2-26 |
| 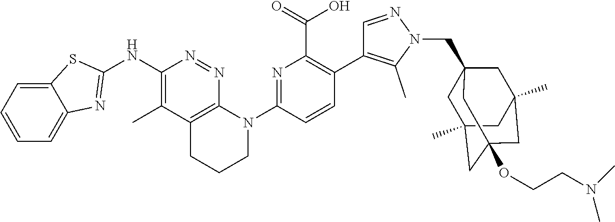 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)C)(C5)C4)c2C)c(C(=O)O)n1] | D2-27 |
| 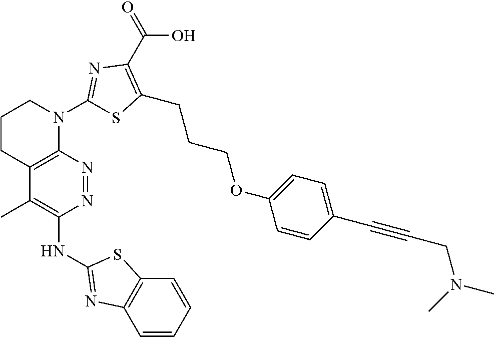 [SMILES: Cc1c(Nc2nc3ccccc3s2)nnc2c1CCCN2c1nc(C(=O)O)c(CCCOc2ccc(C#CCN(C)C)cc2)s1] | D2-28 |
| 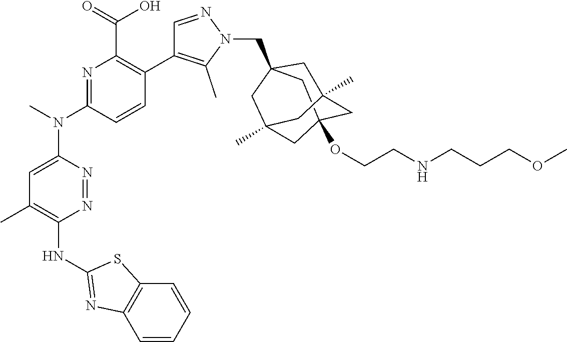 | D2-29 |
| 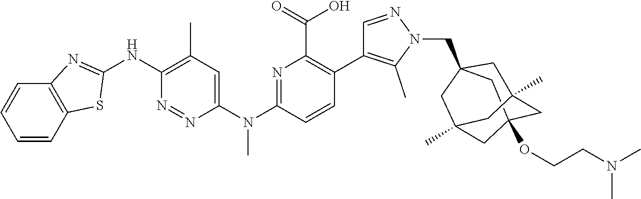 [SMILES: Cc1cc(N(C)c2ccc(-c3cnn(C[C@]45C[C@]6(C)C[C@](C)(C4)C[C@@](OCCN(C)C)(C6)C5)c3C)c(C(=O)O)n2)nnc1Nc1nc2ccccc2s1] | D2-30 |
| 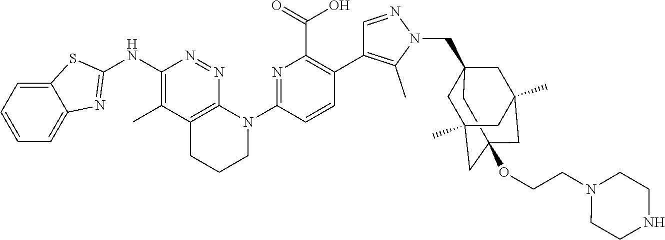 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCNCC3)(C5)C4)c2C)c(C(=O)O)n1] | D2-31 |
| 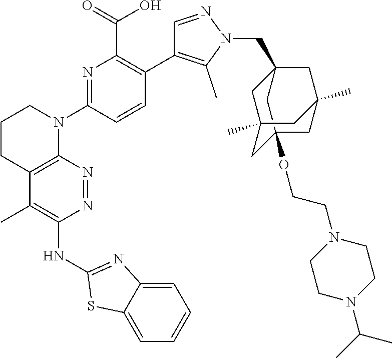 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCN(C(C)C)CC3)(C5)C4)c2C)c(C(=O)O)n1] | D2-32 |
| 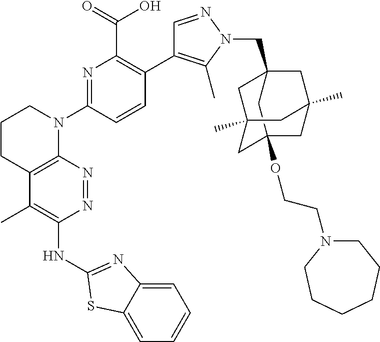 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCCCCC3)(C5)C4)c2C)c(C(=O)O)n1] | D2-33 |
| 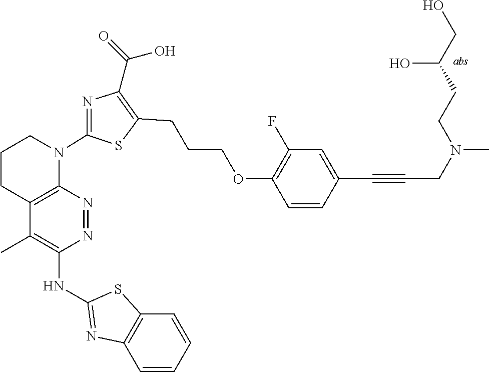 | D2-34 |
| 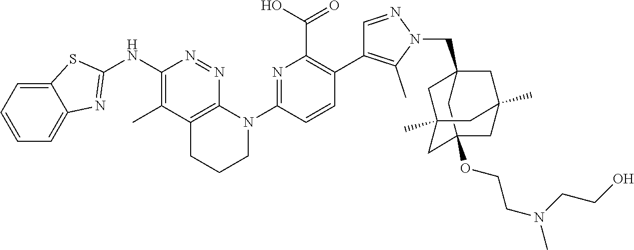 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)CCO)(C5)C4)c2C)c(C(=O)O)n1] | D2-35 |
| 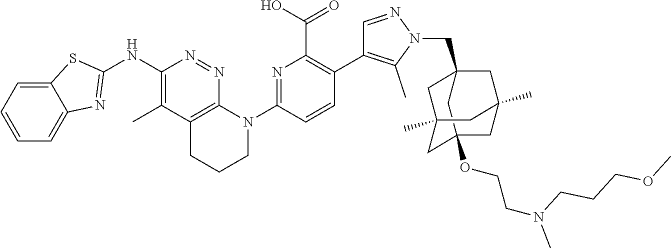 [SMILES: COCCCN(C)CCO[C@]12C[C@@]3(C)C[C@](C)(C[C@](Cn4ncc(-c5ccc(N6CCCc7c6nnc(Nc6nc8ccccc8s6)c7C)nc5C(=O)O)c4C)(C3)C1)C2] | D2-36 |
| 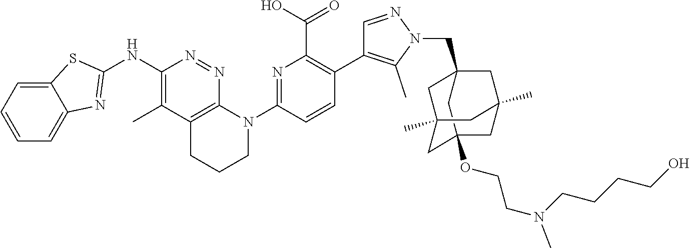 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)CCCCO)(C5)C4)c2C)c(C(=O)O)n1] | D2-37 |
| 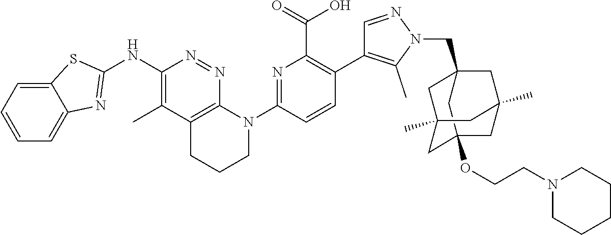 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCCCC3)(C5)C4)c2C)c(C(=O)O)n1] | D2-38 |
| 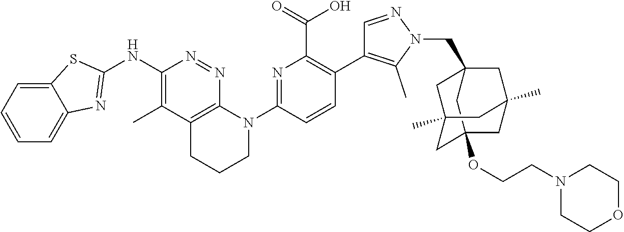 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCOCC3)(C5)C4)c2C)c(C(=O)O)n1] | D2-39 |
| 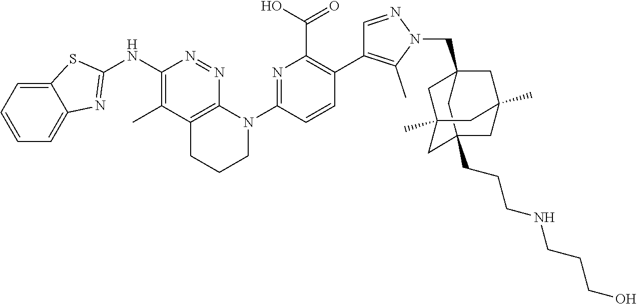 [SMILES: Cc1c(Nc2nc3ccccc3s2)nnc2c1CCCN2c1ccc(-c2cnn(C[C@]34C[C@@]5(C)C[C@](C)(C[C@](CCCNCCCO)(C5)C3)C4)c2C)c(C(=O)O)n1] | D2-40 |
| 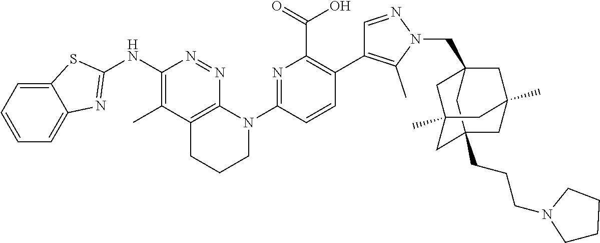 [SMILES: Cc1c(Nc2nc3ccccc3s2)nnc2c1CCCN2c1ccc(-c2cnn(C[C@]34C[C@@]5(C)C[C@](C)(C[C@](CCCN6CCCC6)(C5)C3)C4)c2C)c(C(=O)O)n1] | D2-41 |
| 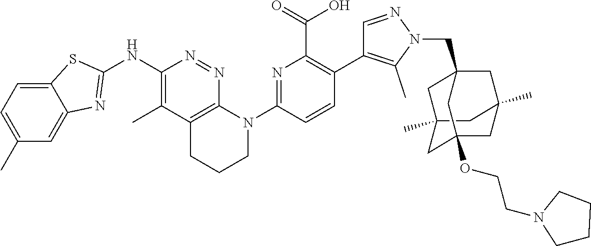 [SMILES: Cc1ccc2sc(Nc3nnc4c(c3C)CCCN4c3ccc(-c4cnn(C[C@]56C[C@]7(C)C[C@](C)(C5)C[C@@](OCCN5CCCC5)(C7)C6)c4C)c(C(=O)O)n3)nc2c1] | D2-42 |
| 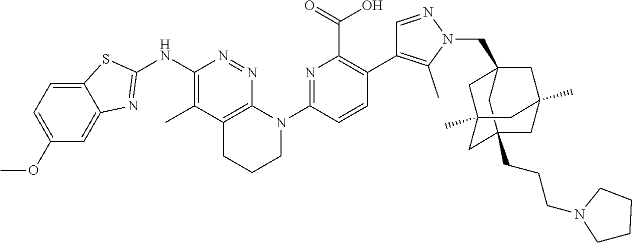 [SMILES: COc1ccc2sc(Nc3nnc4c(c3C)CCCN4c3ccc(-c4cnn(C[C@]56C[C@@]7(C)C[C@](C)(C[C@](CCCN8CCCC8)(C7)C5)C6)c4C)c(C(=O)O)n3)nc2c1] | D2-43 |
| 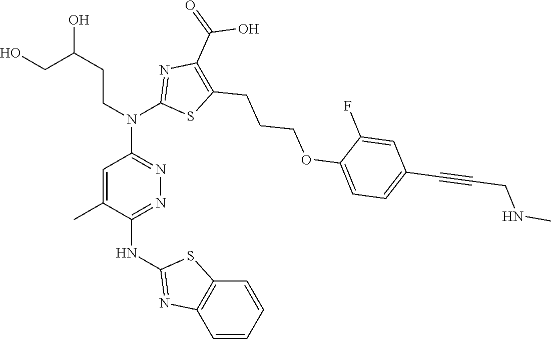 [SMILES: CNCC#Cc1ccc(OCCCc2sc(N(CCC(O)CO)c3cc(C)c(Nc4nc5ccccc5s4)nn3)nc2C(=O)O)c(F)c1] | D2-44 |
| 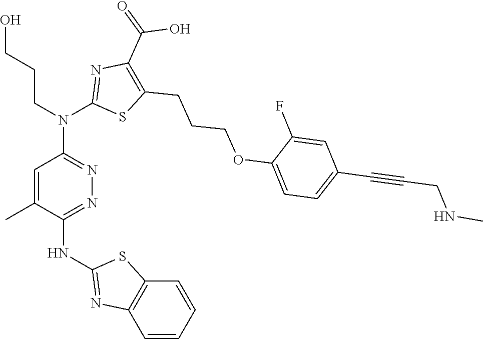 | D2-45 |
| 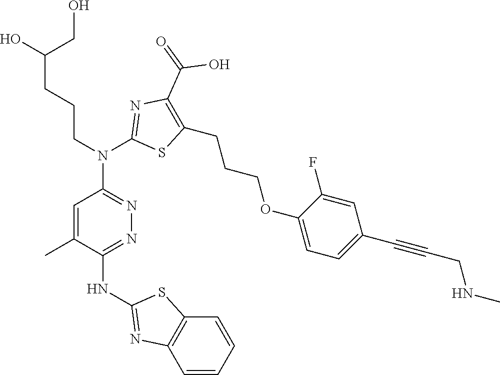 [SMILES: CNCC#Cc1ccc(OCCCc2sc(N(CCCC(O)CO)c3cc(C)c(Nc4nc5ccccc5s4)nn3)nc2C(=O)O)c(F)c1] | D2-46 |
| 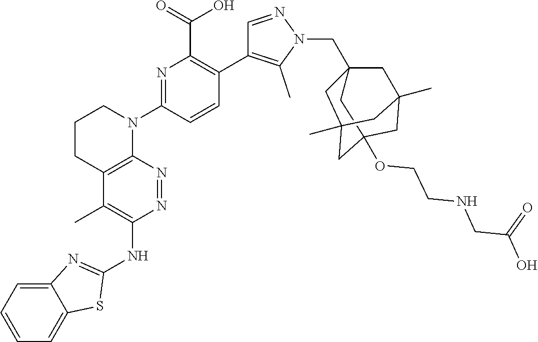 [SMILES: Cc1c(Nc2nc3ccccc3s2)nnc2c1CCCN2c1ccc(-c2cnn(CC34CC5(C)CC(C)(C3)CC(OCCNCC(=O)O)(C5)C4)c2C)c(C(=O)O)n1] | D2-47 |
| 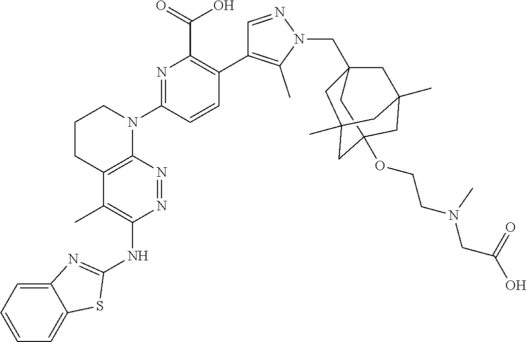 [SMILES: Cc1c(Nc2nc3ccccc3s2)nnc2c1CCCN2c1ccc(-c2cnn(CC34CC5(C)CC(C)(C3)CC(OCCN(C)CC(=O)O)(C5)C4)c2C)c(C(=O)O)n1] | D2-48 |
| 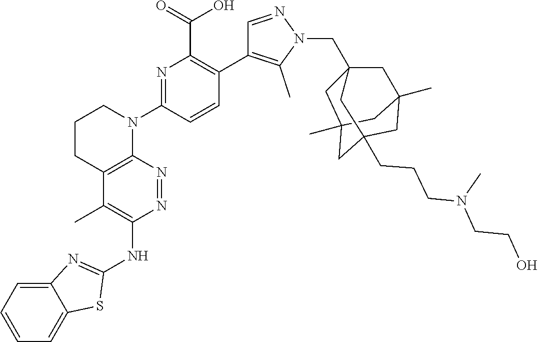 [SMILES: Cc1c(Nc2nc3ccccc3s2)nnc2c1CCCN2c1ccc(-c2cnn(CC34CC5(C)CC(C)(CC(CCCN(C)CCO)(C5)C3)C4)c2C)c(C(=O)O)n1] | D2-49 |
| 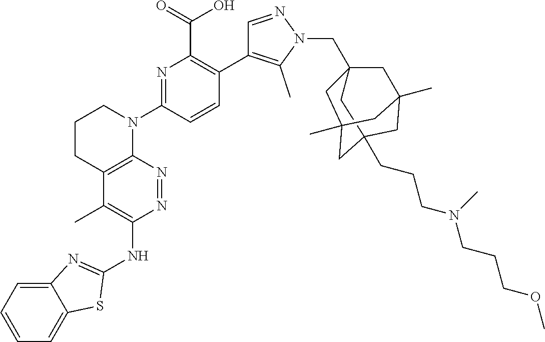 [SMILES: COCCCN(C)CCCC12CC3(C)CC(C)(C1)CC(Cn1ncc(-c4ccc(N5CCCc6c5nnc(Nc5nc7ccccc7s5)c6C)nc4C(=O)O)c1C)(C3)C2] | D2-50 |
| 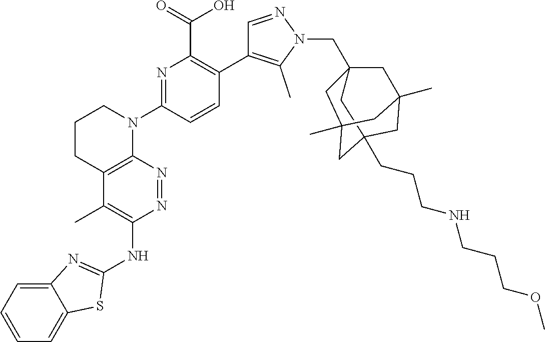 [SMILES: COCCCNCCCC12CC3(C)CC(C)(C1)CC(Cn1ncc(-c4ccc(N5CCCc6c5nnc(Nc5nc7ccccc7s5)c6C)nc4C(=O)O)c1C)(C3)C2] | D2-51 |
| 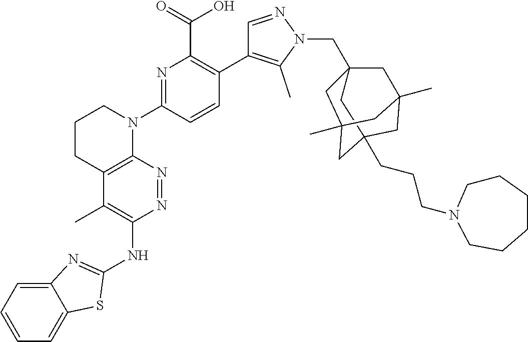 [SMILES: Cc1c(Nc2nc3ccccc3s2)nnc2c1CCCN2c1ccc(-c2cnn(CC34CC5(C)CC(C)(CC(CCCN6CCCCCC6)(C5)C3)C4)c2C)c(C(=O)O)n1] | D2-52 |
| 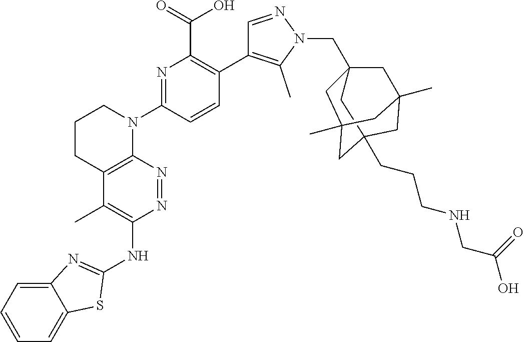 [SMILES: Cc1c(Nc2nc3ccccc3s2)nnc2c1CCCN2c1ccc(-c2cnn(CC34CC5(C)CC(C)(CC(CCCNCC(=O)O)(C5)C3)C4)c2C)c(C(=O)O)n1] | D2-53 |
| 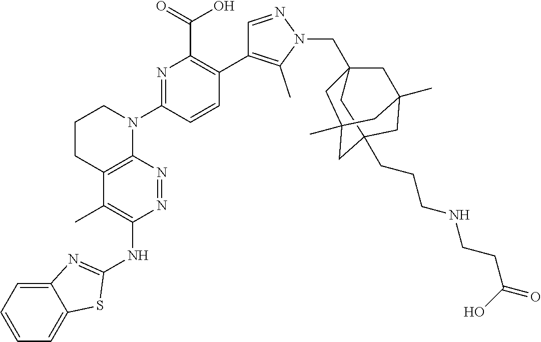 [SMILES: Cc1c(Nc2nc3ccccc3s2)nnc2c1CCCN2c1ccc(-c2cnn(CC34CC5(C)CC(C)(CC(CCCNCCC(=O)O)(C5)C3)C4)c2C)c(C(=O)O)n1] | D2-54 |
| 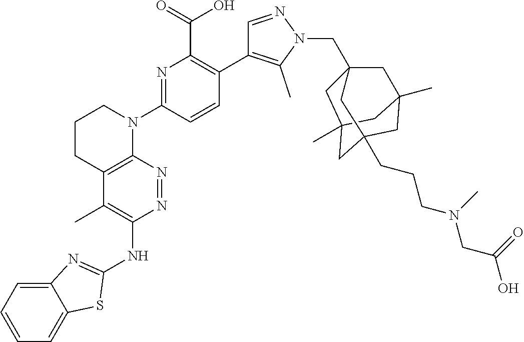 [SMILES: Cc1c(Nc2nc3ccccc3s2)nnc2c1CCCN2c1ccc(-c2cnn(CC34CC5(C)CC(C)(CC(CCCN(C)CC(=O)O)(C5)C3)C4)c2C)c(C(=O)O)n1] | D2-55 |
| 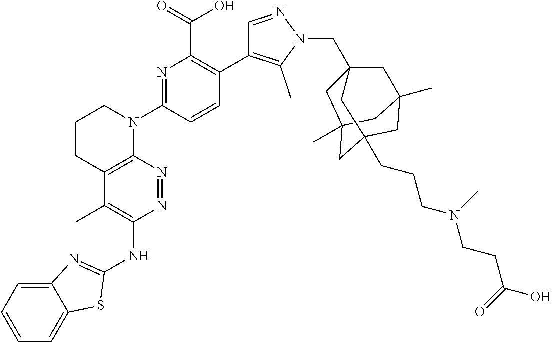 [SMILES: Cc1c(Nc2nc3ccccc3s2)nnc2c1CCCN2c1ccc(-c2cnn(CC34CC5(C)CC(C)(CC(CCCN(C)CCC(=O)O)(C5)C3)C4)c2C)c(C(=O)O)n1] | D2-56 |
| 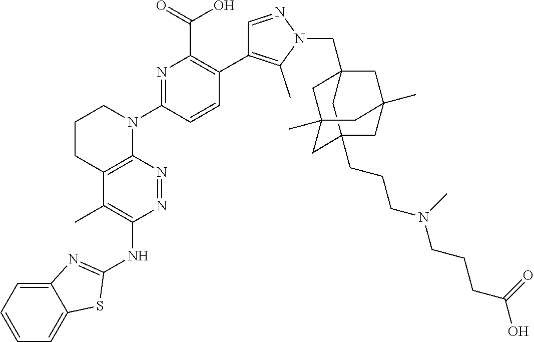 [SMILES: Cc1c(Nc2nc3ccccc3s2)nnc2c1CCCN2c1ccc(-c2cnn(CC34CC5(C)CC(C)(CC(CCCN(C)CCCC(=O)O)(C5)C3)C4)c2C)c(C(=O)O)n1] | D2-57 |
| 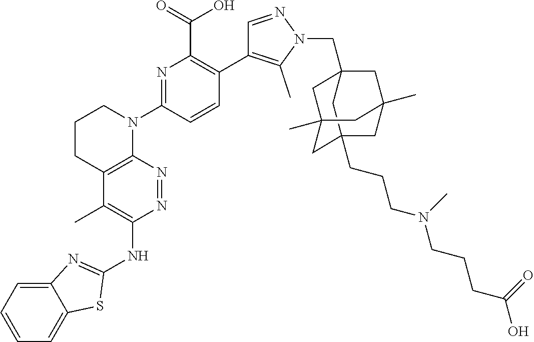 [SMILES: Cc1c(Nc2nc3ccccc3s2)nnc2c1CCCN2c1ccc(-c2cnn(CC34CC5(C)CC(C)(CC(CCCN(C)CCCC(=O)O)(C5)C3)C4)c2C)c(C(=O)O)n1] | D2-58 |
| 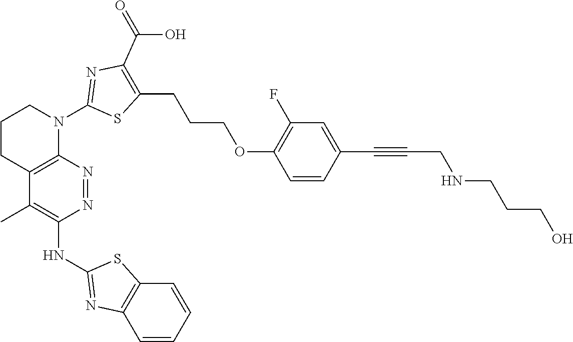 [SMILES: Cc1c(Nc2nc3ccccc3s2)nnc2c1CCCN2c1nc(C(=O)O)c(CCCOc2ccc(C#CCNCCCO)cc2F)s1] | D2-59 |
| 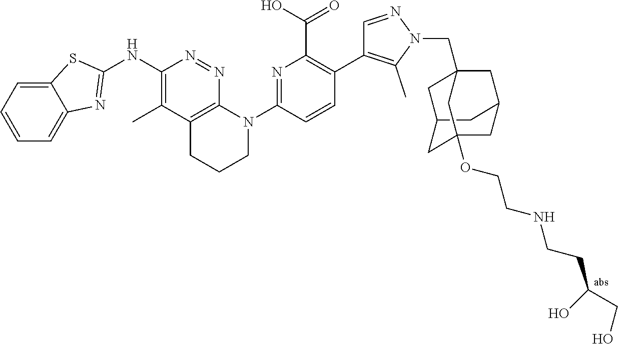 | D2-60 |
| 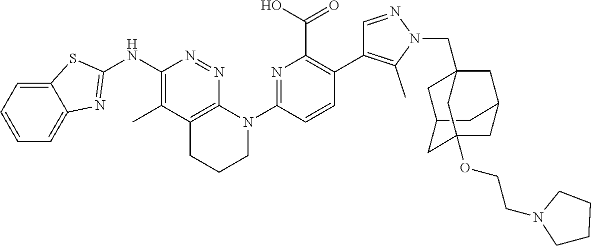 [SMILES: Cc1c(Nc2nc3ccccc3s2)nnc2c1CCCN2c1ccc(-c2cnn(CC34CC5CC(C3)CC(OCCN3CCCC3)(C5)C4)c2C)c(C(=O)O)n1] | D2-61 |
| 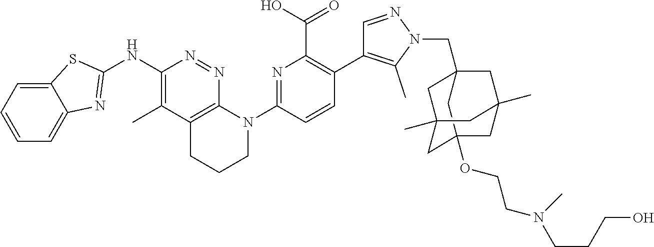 [SMILES: Cc1c(Nc2nc3ccccc3s2)nnc2c1CCCN2c1ccc(-c2cnn(CC34CC5(C)CC(C)(C3)CC(OCCN(C)CCCO)(C5)C4)c2C)c(C(=O)O)n1] | D2-62 |
| 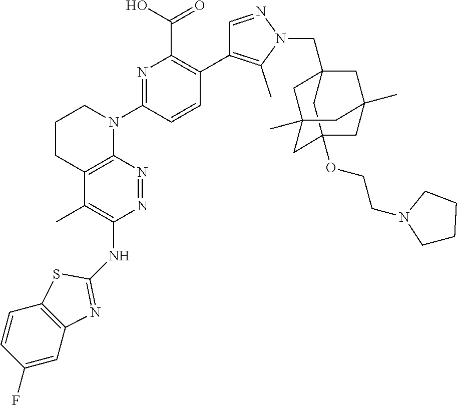 [SMILES: Cc1c(Nc2nc3cc(F)ccc3s2)nnc2c1CCCN2c1ccc(-c2cnn(CC34CC5(C)CC(C)(C3)CC(OCCN3CCCC3)(C5)C4)c2C)c(C(=O)O)n1] | D2-63 |
| 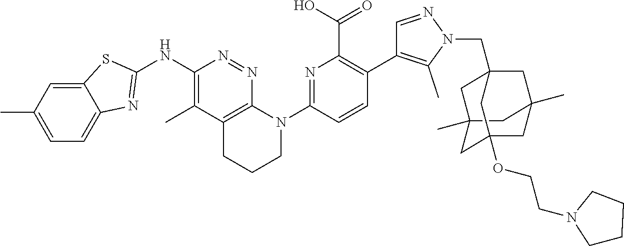 [SMILES: Cc1ccc2nc(Nc3nnc4c(c3C)CCCN4c3ccc(-c4cnn(CC56CC7(C)CC(C)(C5)CC(OCCN5CCCC5)(C7)C6)c4C)c(C(=O)O)n3)sc2c1] | D2-64 |
| 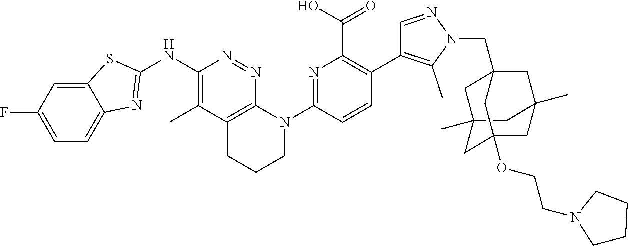 [SMILES: Cc1c(Nc2nc3ccc(F)cc3s2)nnc2c1CCCN2c1ccc(-c2cnn(CC34CC5(C)CC(C)(C3)CC(OCCN3CCCC3)(C5)C4)c2C)c(C(=O)O)n1] | D2-65 |
| 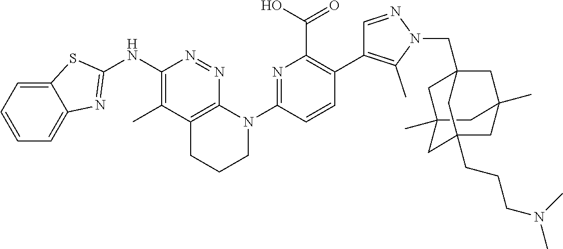 [SMILES: Cc1c(Nc2nc3ccccc3s2)nnc2c1CCCN2c1ccc(-c2cnn(CC34CC5(C)CC(C)(CC(CCCN(C)C)(C5)C3)C4)c2C)c(C(=O)O)n1] | D2-66 |
| 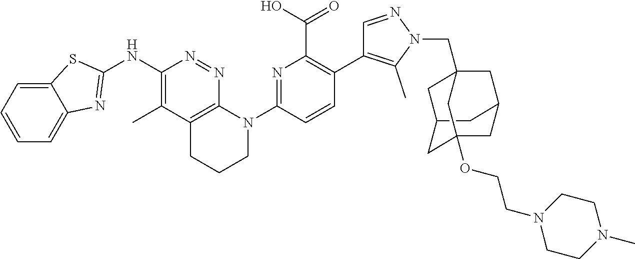 [SMILES: Cc1c(Nc2nc3ccccc3s2)nnc2c1CCCN2c1ccc(-c2cnn(CC34CC5CC(C3)CC(OCCN3CCN(C)CC3)(C5)C4)c2C)c(C(=O)O)n1] | D2-67 |
| 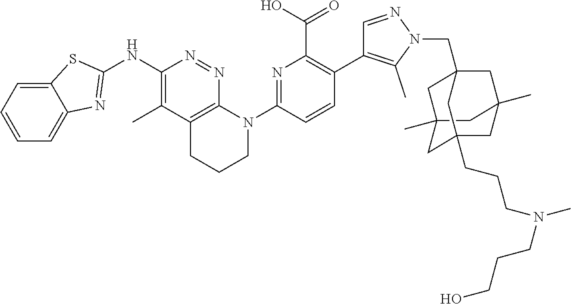 [SMILES: Cc1c(Nc2nc3ccccc3s2)nnc2c1CCCN2c1ccc(-c2cnn(CC34CC5(C)CC(C)(CC(CCCN(C)CCCO)(C5)C3)C4)c2C)c(C(=O)O)n1] | D2-68 |
| 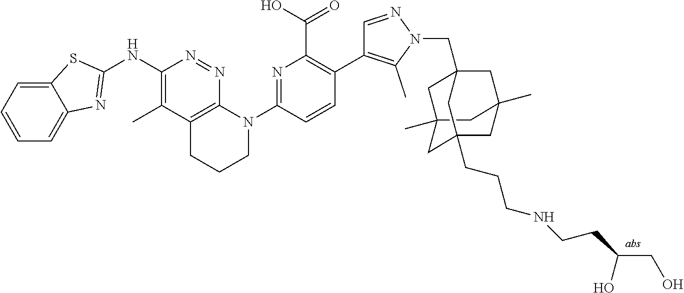 [SMILES: Cc1c(Nc2nc3ccccc3s2)nnc2c1CCCN2c1ccc(-c2cnn(CC34CC5(C)CC(C)(CC(CCCNCC[C@H](O)CO)(C5)C3)C4)c2C)c(C(=O)O)n1] | D2-69 |
| 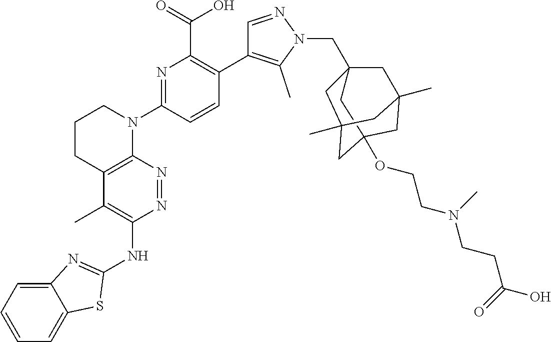 [SMILES: Cc1c(Nc2nc3ccccc3s2)nnc2c1CCCN2c1ccc(-c2cnn(CC34CC5(C)CC(C)(C3)CC(OCCN(C)CCC(=O)O)(C5)C4)c2C)c(C(=O)O)n1] | D2-70 |
| 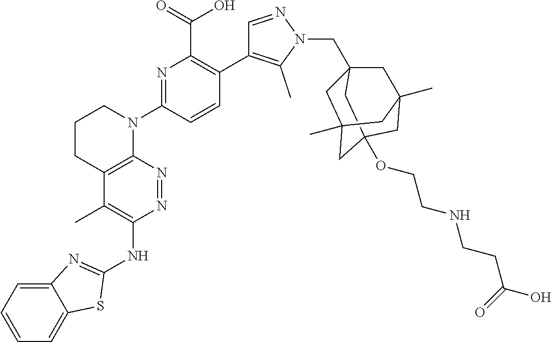 [SMILES: Cc1c(Nc2nc3ccccc3s2)nnc2c1CCCN2c1ccc(-c2cnn(CC34CC5(C)CC(C)(C3)CC(OCCNCCC(=O)O)(C5)C4)c2C)c(C(=O)O)n1] | D2-71 |
| 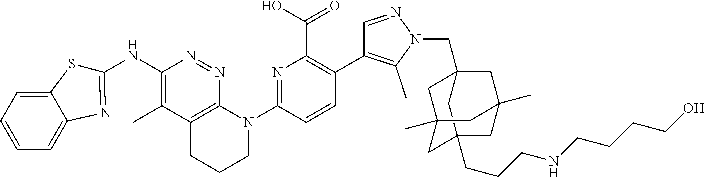 [SMILES: Cc1c(Nc2nc3ccccc3s2)nnc2c1CCCN2c1ccc(-c2cnn(CC34CC5(C)CC(C)(CC(CCCNCCCCO)(C5)C3)C4)c2C)c(C(=O)O)n1] | D2-72 |
| 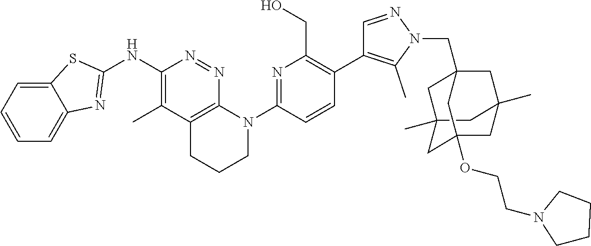 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(CO)n1] | D2-73 |
| 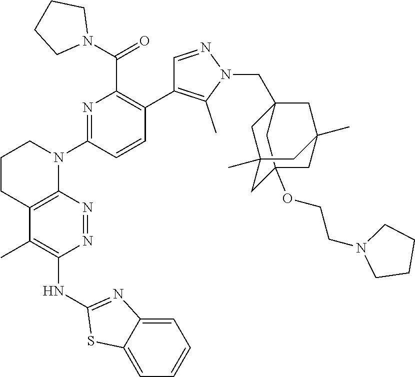 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)N2CCCC2)n1] | D2-74 |
| 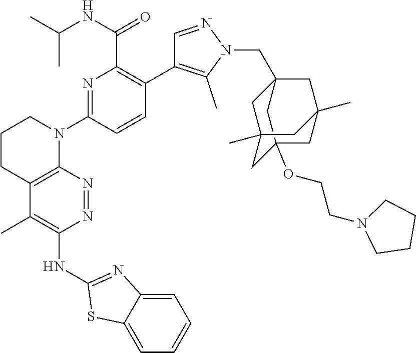 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)NC(C)C)n1] | D2-75 |
| 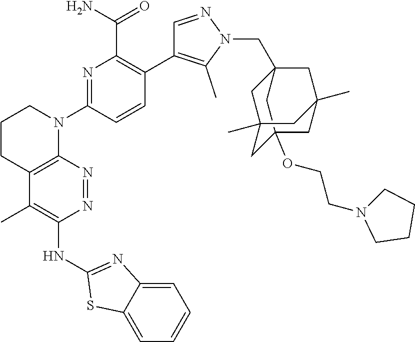 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(N)=O)n1] | D2-76 |

or an enantiomer, a diastereoisomer, and/or a pharmaceutically acceptable salt of any one of the foregoing. The definitions of the remaining variables are provided in the sixty-fifth embodiment or any embodiments described therein.

In a seventy-sixth embodiment, the present disclosure provides an antibody-drug conjugate of any one of the forty-sixth through fiftieth embodiments, or pharmaceutically acceptable salt thereof, wherein the Bcl-2 inhibitor is represented by Formula (IV) or Formula (V):

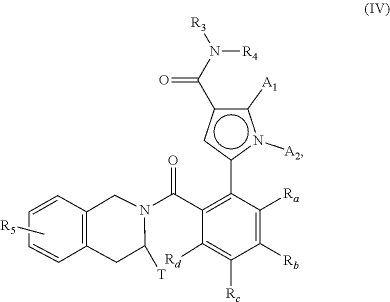

or an enantiomer, a diastereoisomer, and/or a pharmaceutically acceptable salt of any one of the foregoing, wherein:
A₁ represents a hydrogen or halogen atom, a linear or branched (C₁-C₆)polyhaloalkyl group, a linear or branched (C₁-C₆)alkyl group or a cycloalkyl group,
A₂ represents a linear or branched (C₁-C₆)alkyl group optionally substituted by a group selected from halogen, hydroxy, linear or branched (C₁-C₆)alkoxy, NR′R″ and morpholine, or A₂ represents a linear or branched (C₁-C₆)polyhaloalkyl group or a cyclopropyl group,
it being understood that R¹ and R″, each independently of the other, represent a hydrogen atom or a linear or branched (C₁-C₆)alkyl group,
T represents a hydrogen atom, a linear or branched (C₁-C₆)alkyl group optionally substituted by from one to three halogen atoms, a group (C₁-C₄)alkyl-NR₁R₂, or a group (C₁-C₄)alkyl-OR₆, R₁ and R₂, each independently of the other, represent a hydrogen atom or a linear or branched (C₁-C₆)alkyl group,
or R₁ and R₂ form with the nitrogen atom carrying them a heterocycloalkyl,
R₃ represents an aryl or heteroaryl group, it being understood that one or more carbon atoms of the preceding groups, or of their possible substituents, may be deuterated,
R₄ represents a phenyl group, a 4-hydroxyphenyl group, a 3-fluoro-4-hydroxyphenyl group, a 2-hydroxypyrimidine group or a 3-hydroxypyridine group, it being understood that one or more carbon atoms of the preceding groups, or of their possible substituents, may be deuterated, R₅ represents a hydrogen or halogen atom, a linear or branched (C₁-C₆)alkyl group, or a linear or branched (C₁-C₆)alkoxy group,
R₆ represents a hydrogen atom or a linear or branched (C₁-C₆)alkyl group,
Rₐ and R_{d} each represent a hydrogen atom and (R_{b},R_{c}) form together with the carbon atoms carrying them a 1,3-dioxolane group or a 1,4-dioxane group, or Rₐ, R_{c} and R_{d} each represent a hydrogen atom and R_{b} represents a hydrogen or halogen atom or a methoxy group,
or Rₐ and R_{d} each represent a hydrogen atom, R_{b} represents a hydrogen or halogen atom and R_{c}represents a hydroxy or methoxy group, or: Rₐ and R_{d} each represent a hydrogen atom, R_{b}represents a hydroxy or methoxy group and R_{c} represents a halogen atom, or

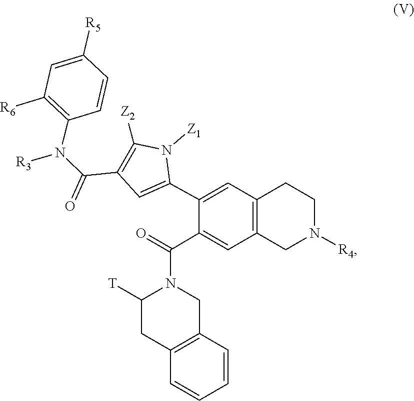

or an enantiomer, a diastereoisomer, and/or a pharmaceutically acceptable salt of any one of the foregoing, wherein:
Z₁ and Z₂ represent both a methyl group or they form together with the atoms carrying them a fused piperidine group,
T represents a hydrogen atom, a linear or branched (C₁-C₆)alkyl group optionally substituted by one to three halogen atoms, a (C₁-C₄)alkylene-NR₁R₂ group, a (C₁-C₄)alkylene-ORᵢ group, R₁ and R₂ independently of one another represent a hydrogen atom or a linear or branched (C₁-C₆)alkyl group,
or R₁ and R₂ form with the nitrogen atom carrying them a heterocycloalkyl group, which heterocycloalkyl is optionally substituted by one to three groups selected from: (C₁-C₆)alkyl group and halogen atom,
R₃ represents a group selected from:

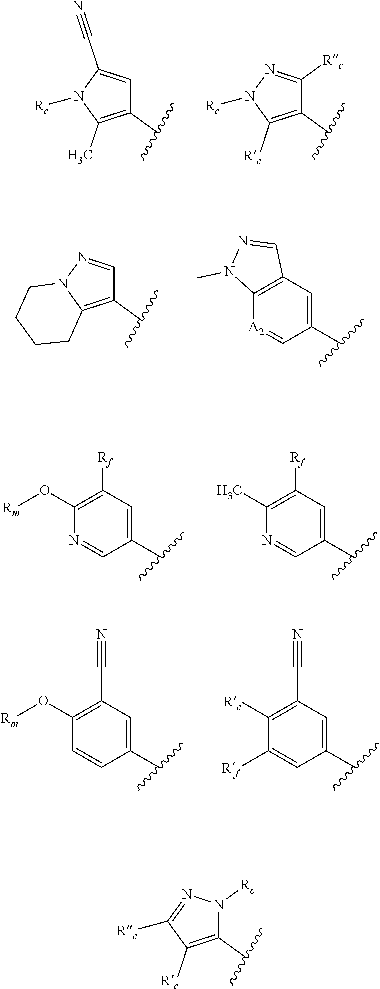

R₄ represents a group selected from:

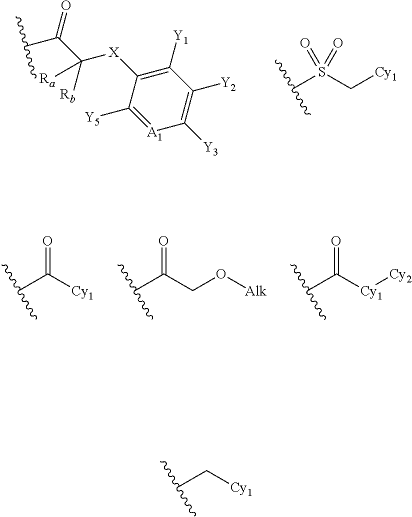

R₅ represents a hydrogen atom, a halogen atom or a hydroxy group,
R₆ represents a hydrogen, a linear or branched (C₁-C₆)alkyl group, or a halogen atom,
Alk represents a linear or branched (C₁-C₆)alkyl group,
A₁ represents a C—Y₄ or a nitrogen atom,
A₂ represents a C—H or a nitrogen atom,
Cy₁ represents a phenyl, a heteroaryl, a cycloalkyl or a heterocycloalkyl group, wherein the phenyl, the heteroaryl, the cycloalkyl and the heterocycloalkyl groups are optionally substituted by one to three groups selected from: linear or branched (C₁-C₆)alkyl group optionally substituted by 1 to 3 halogen atoms, hydroxy group, cycloalkyl group, and halogen atom and the heterocycloalkyl group is optionally further substituted by an oxo group,
Cy₂ represent a phenyl or a heteroaryl group, wherein the phenyl and the heteroaryl groups are optionally substituted by one to three groups selected from: linear or branched (C₁-C₆)alkyl group optionally substituted by 1 to 3 halogen atoms, hydroxy group, and halogen atom X represents a bond, —O—, —S— or NRₖ,
Y₁ and Y₅ independently of one another represent a group selected from: hydrogen atom, halogen atom, cyano, linear or branched (C₁-C₆)alkyl group, and linear or branched (C₁-C₆)alkoxy group,
Y₂ and Y₄ independently of one another represent a group selected from: hydrogen atom, halogen atom, linear or branched (C₁-C₆)alkyl group, linear or branched (C₁-C₆)alkoxy group, and heterocycloalkyl group optionally substituted by a linear or branched (C₁-C₆)alkyl group, Y₃ represents a group selected from: hydrogen atom, halogen atom, linear or branched (C₁-C₆)alkyl, linear or branched (C₁-C₆)alkynyl, —(C₁-C₄)alkylene-OR_{I}, linear or branched (C₁-C₆)alkoxy group, —O-phenyl, —S-phenyl, —O—(C₁-C₄)alkylene-Cy₃, —O—(C₁-C₄)alkylene-Cy₄, —O-Cy3, —O—(C₁-C₄)alkylene-NR_{g}Rh, —(C₁-C₄)alkylene-Cy₃, —(C₁-C₄)alkylene-Cy₄, Cy3, Cy4, and:

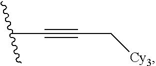

wherein the alkylene moiety of the preceding groups may be linear or branched, Cy₃ represents a heterocycloalkyl optionally substituted by one to three groups selected from: linear or branched (C₁-C₆)alkyl group optionally substituted by 1 to 3 halogen atoms, hydroxy group, cycloalkyl group, heterocycloalkyl group, and halogen atom,
Cy₄ represents a cycloalkyl optionally substituted by one to three groups selected from: linear or branched (C₁-C₆)alkyl group optionally substituted by 1 to 3 halogen atoms, hydroxy group, cycloalkyl group, heterocycloalkyl group, and halogen atom
Rₐ and R_{b} independently of one another represent a hydrogen atom or a halogen atom, R_{c} represents a group selected from: hydrogen, linear or branched (C₁-C₆)alkyl group optionally substituted by 1 to 3 halogen atoms, (C₁-C₆)alkylene-NRₐRₑ, (C₁-C₆)alkylene-ORⱼ, cycloalkyl, heterocycloalkyl, and (C₁-C₆)alkylene-heterocycloalkyl group,
R′_{c} and R″_{c} independently of one another represent a hydrogen atom or a linear or branched (C₁-C₆)alkyl (preferably a methyl),
R_{d} and Rₑ independently of one another represent a hydrogen atom, a linear or branched (C₁-C₆)alkyl group, a cycloalkyl group or a heterocycloalkyl group,
R_{f} represents a hydrogen atom, a halogen atom or a cyano group,
R¹ represents a hydrogen atom or a halogen atom,
R_{g} and Rₕ independently of one another represent a hydrogen atom, a linear or branched (C₁-C₆)alkyl group optionally substituted by one to three halogen atoms, a cycloalkyl group, a heterocycloalkyl group, or a —(C₁-C₆)alkylene-heterocycloalkyl,
Rᵢ, Rⱼ, and Rₖ independently of one another represent a hydrogen atom, a linear or branched (C₁-C₆)alkyl group, or a —(C₁-C₆)alkylene-cycloalkyl group,
R₁ represents a hydrogen atom, a linear or branched (C₁-C₆)alkyl group or a linear or branched (C₁-C₆)alkylene-heterocycloalkyl group,
Rm represents a hydrogen or a linear or branched (C₁-C₆)alkyl group.

The definitions of the remaining variables are provided in the forty-sixth through fiftieth embodiments or any embodiments described therein.

In some embodiment, it is understood that:
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen (including quaternary nitrogens),
“cycloalkyl” means any mono- or bi-cyclic, non-aromatic, carbocyclic group containing from 3 to 10 ring members,
“heterocycloalkyl” means any mono- or bi-cyclic, non-aromatic, condensed or spiro group composed of from 3 to 10 ring members and containing from 1 to 3 hetero atoms selected from oxygen, sulphur, SO, SO₂ and nitrogen, and
it is possible for the aryl, heteroaryl, cycloalkyl and heterocycloalkyl groups so defined and the groups alkyl, alkenyl, alkynyl and alkoxy to be substituted by from 1 to 3 groups selected from linear or branched (C₁-C₆)alkyl, (C₃-C₆)spiro, linear or branched (C₁-C₆)alkoxy, (C₁-C₆)alkyl-S—, hydroxy, oxo (or N-oxide where appropriate), nitro, cyano, —COOR′, —OCOR′, NR′R″, linear or branched (C₁-C₆)polyhaloalkyl, trifluoromethoxy, (C1C6)alkylsulphonyl, halogen, aryl, heteroaryl, aryloxy, arylthio, cycloalkyl, heterocycloalkyl optionally substituted by one or more halogen atoms or alkyl groups,
In a seventy-seventh embodiment, the present disclosure provides an antibody-drug conjugate of the seventy-sixth embodiment, or a pharmaceutically acceptable salt thereof, wherein the Bcl-2 inhibitor is represented by Formula (IV) or an enantiomer, a diastereoisomer, and/or a pharmaceutically acceptable salt of any one of the foregoing. The definitions of the remaining variables are provided in the seventy-sixth embodiment or any embodiments described therein.

In a seventy-eighth embodiment, the present disclosure provides an antibody-drug conjugate of the seventy-sixth or seventy-seventh embodiment, wherein, in Formula (IV), (i) A₁ represents a hydrogen atom or a methyl group; or (ii) A₁ and A₂ both represent a methyl group. The definitions of the remaining variables are provided in the seventy-sixth or seventy-seventh embodiment or any embodiments described therein.

In a seventy-ninth embodiment, the present disclosure provides an antibody-drug conjugate of the seventy-sixth through seventy-eighth embodiments, wherein, in Formula (IV), T represents a methyl, aminomethyl, (morpholin-4-yl)methyl, (4-methylpiperazin-1-yl)methyl, 2-(morpholin-4-yl)ethyl, [2-(morpholin-4-yl)ethoxy]methyl, hydroxymethyl, [2-(dimethylamino)ethoxy]methyl, hexahydropyrazino[2,1-c][1,4]oxazin-8(1H)-ylmethyl, 1-oxa-6-azaspiro[3.3]hept-6-ylmethyl, 3-(morpholin-4-yl)propyl or trifluoromethyl group. The definitions of the remaining variables are provided in the seventy-sixth through seventy-eighth embodiments or any embodiments described therein.

In a eightieth embodiment, the present disclosure provides an antibody-drug conjugate of any one of the seventy-sixth through seventy-ninth embodiments, wherein, in Formula (IV), R₃ represents a group selected from phenyl, 1H-pyrazole, 1H-indole, 1H-indazole, pyridine, pyrimidine, 1H-pyrrolo[2,3-b]pyridine, 2,3-dihydro-1H-pyrrolo[2,3-b]pyridine, 1H-benzimidazole, 1H-pyrrole, 1H-pyrrolo[2,3-c]pyridine, 1H-pyrrolo[3,2-b]pyridine, 5H-pyrrolo[3,2-d]pyrimidine, thiophene, pyrazine, 1H-pyrazolo[3,4-b]pyridine, 1,2-oxazole, and pyrazolo[1,5-a]pyrimidine, those groups optionally having one or more substituents selected from halogen, linear or branched (C₁-C₆)alkyl, linear or branched (C₁C₆)alkoxy, cyano, cyclopropyl, oxetane, tetrahydrofuran, —COO—CH₃, trideuteriomethyl, 2-(morpholin-4-yl)ethyl and 2-(morpholin-4-yl)ethoxy. The definitions of the remaining variables are provided in the seventy-sixth through seventy-ninth embodiments or any embodiments described therein.

In an eighty-first embodiment, the present disclosure provides an antibody-drug conjugate of the seventy-sixth embodiment, wherein the Bcl-2 inhibitor is represented by Formula (V) or an enantiomer, a diastereoisomer, and/or a pharmaceutically acceptable salt of any one of the foregoing. The definitions of the remaining variables are provided in the seventy-sixth embodiment or any embodiments described therein.

In an eighty-second embodiment, the present disclosure provides an antibody-drug conjugate of the seventy-sixth embodiment, wherein the Bcl-2 inhibitor is represented by Formula (Va):

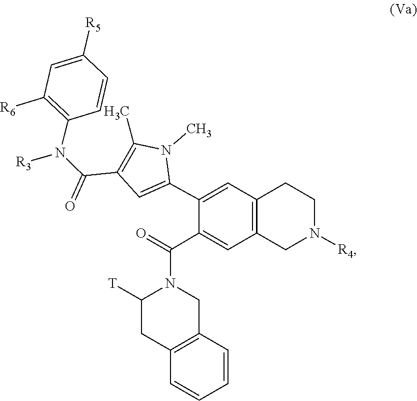

or an enantiomer, a diastereoisomer, and/or a pharmaceutically acceptable salt of any one of the foregoing. The definitions of the remaining variables are provided in the seventy-sixth embodiment or any embodiments described therein.

In an eighty-third embodiment, the present disclosure provides an antibody-drug conjugate of the eighty-first or eighty-second embodiment, wherein R₃ in Formula (V) or (Va) represents the following group:

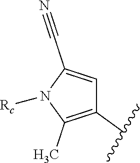

and R_{c} represents a group selected from: hydrogen, linear or branched (C₁-C₆)alkyl group optionally substituted by 1 to 3 halogen atoms, (C₁-C₆)alkylene-NRₐRₑ, (C₁-C₆)alkylene-ORⱼ, cycloalkyl, heterocycloalkyl, and (C₁-C₆)alkylene-heterocycloalkyl group. The definitions of the remaining variables are provided in the eighty-first or eighty-second embodiment or any embodiments described therein.

In an eighty-fourth embodiment, the present disclosure provides an antibody-drug conjugate of the eighty-third embodiment, wherein R_{c} represents a methyl group. The definitions of the remaining variables are provided in the eighty-third embodiment or any embodiments described therein.

In a eighty-fifth embodiment, the present disclosure provides an antibody-drug conjugate of any one of the eighty-first through eighty-third embodiments, wherein R₄ in Formula (V) or (Va) represents the following group:

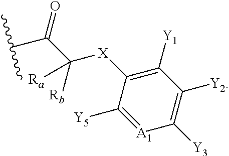

The definitions of the remaining variables are provided in the eighty-first through eighty-third embodiments or any embodiments described therein.

In a eighty-sixth embodiment, the present disclosure provides a process antibody-drug conjugate of the eighty-first embodiment, wherein the Bcl-2 inhibitor is represented by Formula (Vb):

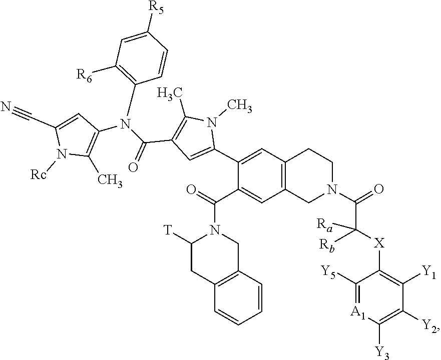

or an enantiomer, a diastereoisomer, and/or a pharmaceutically acceptable salt of any one of the foregoing. The definitions of the remaining variables are provided in the eighty-first embodiment or any embodiments described therein.

In an eighty-seventh embodiment, the present disclosure provides an antibody-drug conjugate of the eighty-sixth embodiment, wherein R_{c} in Formula (Vb) represents a methyl group. The definitions of the remaining variables are provided in the eighty-sixth embodiment or any embodiments described therein.

In an eighty-eighth embodiment, the present disclosure provides an antibody-drug conjugate of the eighty-first embodiment, wherein the Bcl-2 inhibitor is represented by Formula (Vc), (Vd), (Ve), (Vf), (Vg), (Vh), (Vi) or (Vj):

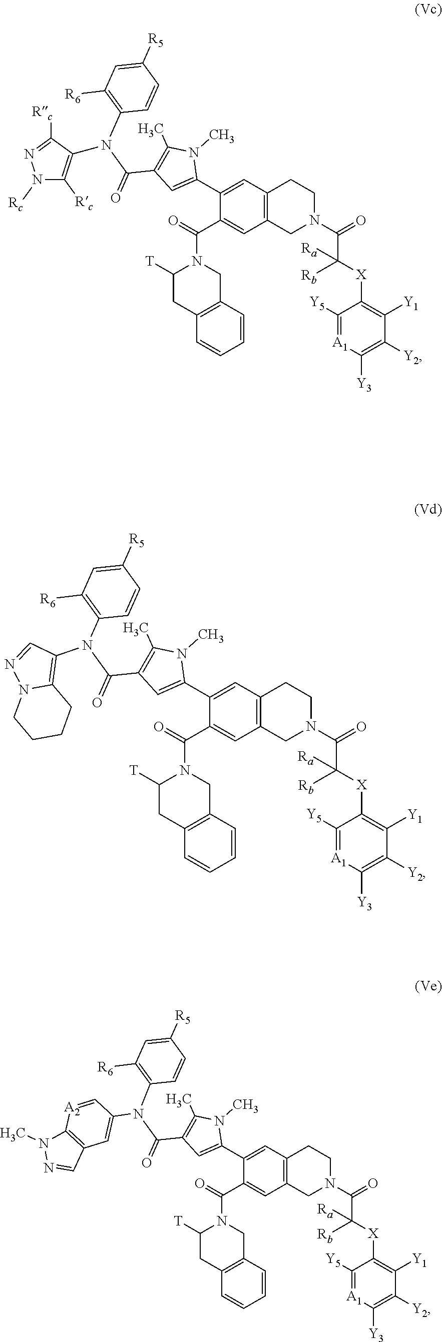

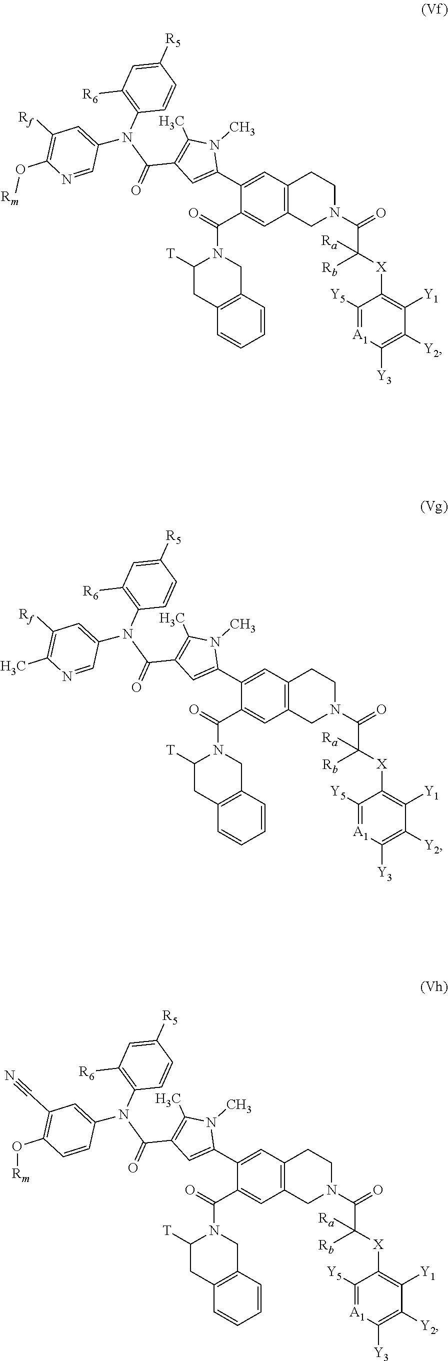

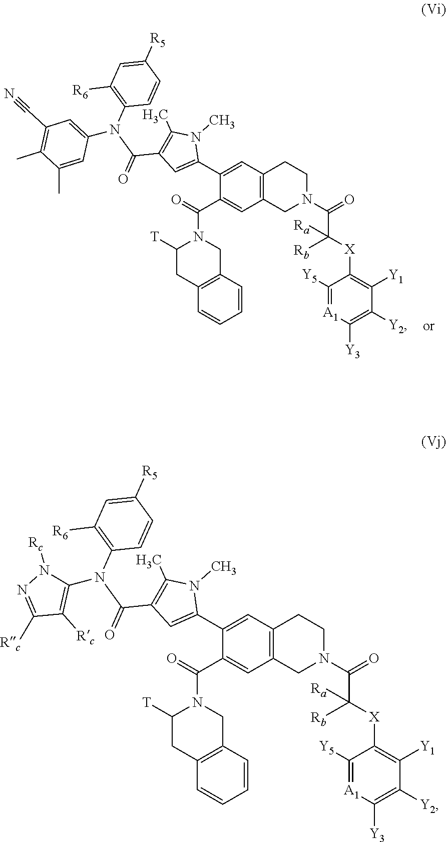

or an enantiomer, a diastereoisomer, and/or a pharmaceutically acceptable salt of any one of the foregoing. The definitions of the remaining variables are provided in the eighty-first embodiment or any embodiments described therein.

In an eighty-ninth embodiment, the present disclosure provides an antibody-drug conjugate of any one of the eighty-first through eighty-eighth embodiments, wherein in Formula (V), (Va), (Vb), (Vc), (Vd), (Ve), (Vf), (Vg), (Vh), (Vi) or (Vj):
(i) X represents a bond;
(ii) A₁ represents C—Y₄;
(iii) Rₐ and R_{b} both represent a hydrogen atom;
(iv) R₅ represents a hydrogen atom, a hydroxy group or a fluorine atom, preferably a hydroxy group;
(v) R₆ represents a hydrogen atom, or a fluorine atom, preferably a hydrogen atom; (vi) A₁ represents C—H and Y₂ represents a hydrogen atom;
(vii) Y₁ and Y₅ represent both a hydrogen atom, or: Y₁ and Y₅ represent a fluoro atom and a hydrogen atom, respectively;
(viii) Y₃ represents a —O—(C₁-C₆)alkylene-heterocycloalkyl group or a —O—(C₁-C₄)alkylene-Cy₃ group;
(ix) Y₃ represents a group selected from: 2-(morpholin-4-yl)ethoxy, 2-(oxan-4-yl)ethoxy, 2-(4-hydroxypiperidin-1-yl)ethoxy, 2-(4-cyclopropylpiperazin-1-yl)ethoxy, 2-[4-(2,2,2-trifluoroethyl)piperazin-1-yl]ethoxy, 2-[(9aS)-octahydropyrazino[2,1-c][1,4]oxazin-8-yl]ethoxy, 2-{2-[4-(2-{1,1-dioxo-lλ⁶-thia-6-azaspiro[3.3]heptan-6-yl}ethoxy, 2-[2,6-dimethylmorpholin-4-yl]ethoxy, 2-[4-(2,2-difluoroethyl)piperazin-1-yl]ethoxy, 2-(3-fluoroazetidin-1-yl)ethoxy, 2-(3,3-difluoropyrrolidin-1-yl)ethoxy, 2-(4-fluoropiperidin-1-yl)ethoxy, 2-(thiomorpholin-4-yl)ethoxy, 2-(2-methylmorpholin-4-yl)ethoxy, 2-{6-oxa-9-azaspiro[4.5]decan-9-yl}ethoxy, 2-{4-oxa-7-azaspiro[2.5]octan-7-yl}ethoxy, 2-[4-(2-fluoroethyl)piperazin-1-yl]ethoxy, 2-(4-methylpiperazin-1-yl)ethoxy, 2-(2,2-dimethylmorpholin-4-yl)ethoxy, 2-(morpholin-4-yl)propoxy, [2-methyl-1-(morpholin-4-yl)propan-2-yl]oxy, 2-(3,3-dimethylmorpholin-4-yl)ethoxy, 2-(3-methylmorpholin-4-yl)ethoxy, 2-(1,4-dioxan-2-yl)ethoxy;
(x) the group:

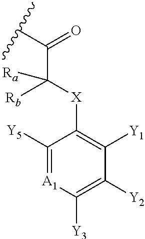

represents

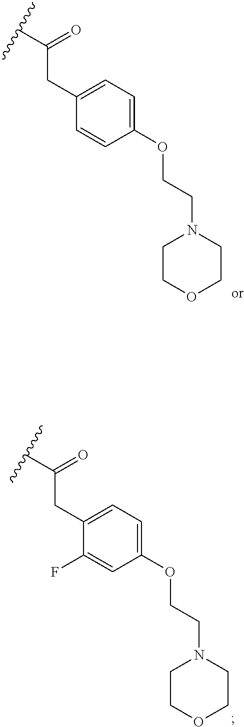 [SMILES: *C(=O)Cc1ccc(OCCN2CCOCC2)cc1 ; *C(=O)Cc1ccc(OCCN2CCOCC2)cc1F]

(xi) T represents a linear or branched (C₁-C₆)alkyl group or a (C₁-C₄)alkylene-NR₁R₂ group; and/or
(xii) T represents a group selected from: methyl group, (piperidin-1-yl)methyl, (morpholin-4-yl)methyl, (piperidin-1-yl)ethyl, [(3R)-3-fluoropyrrolidin-1-yl]methyl, (4-fluoropiperidin-1-yl)methyl, [methyl(propan-2-yl)amino]methyl, (azepan-1-yl)methyl, (pyrrolidin-1-yl)methyl, [(3S)-3-methylpiperidin-1-yl]methyl, [(3R)-3-methylpiperidin-1-yl]methyl, [(1RS,5SR)-3-azabicyclo[3.1.0]hexan-3-yl]methyl, [(2S)-2-methylpiperidin-1-yl]methyl, {6-azaspiro[2.5]octan-6-yl}methyl, (4,4-difluoropiperidin-1-yl)methyl, (diethylamino)methyl, (4-methylpiperidin-1-yl)methyl, [ethyl(propan-2-yl)amino]methyl, {5-azaspiro[2.3]hexan-5-yl}methyl, (3,3-dimethylpyrrolidin-1-yl)methyl, (diisopropylamino)methyl, [ethyl(isopropyl) amino]methyl, [(3R)-3-methylpyrrolidin-1-yl]methyl, [(3S)-3-methylpyrrolidin-1-yl]methyl, [(2S)-2-methylpyrrolidin-1-yl]methyl, 5-azaspiro[2.4]heptan-5-ylmethyl, 2-azaspiro[3.3]heptan-2-ylmethyl, and aminomethyl. The definitions of the remaining variables are provided in the eighty-first through eighty-eighth embodiments or any embodiments described therein.

In some embodiments, for the antibody-drug conjugate of the eighty-eighth embodiment, the Bcl-2 inhibitor is represented by Formula (Vc), (Vd), (Ve), (Vf), (Vg), (Vh), (Vi) or (Vj), wherein:
(i) X represents a bond;
(ii) A₁ represents C—Y₄;
(iii) Rₐ and R_{b} both represent a hydrogen atom;
(iv) R₅ represents a hydrogen atom, a hydroxy group or a fluorine atom, preferably a hydroxy group;
(v) R₆ represents a hydrogen atom, a fluorine atom, preferably a hydrogen atom;
(vi) A₁ represents C—H and Y₂ represents a hydrogen atom;
(vii) Yⱼ and Y₅ represent both a hydrogen atom, or: Yⱼ and Y₅ represent a fluoro atom and a hydrogen atom, respectively;
(viii) Y₃ represents a —O—(C₁-C₆)alkylene-heterocycloalkyl group;
(ix) Y₃ represents a group selected from: 2-(morpholin-4-yl)ethoxy, 2-[4-(2,2-difluoroethyl)piperazin-1-yl]ethoxy, 2-(3-fluoroazetidin-1-yl)ethoxy, 2-(3,3-difluoropyrrolidin-1-yl)ethoxy, 2-(oxan-4-yl)ethoxy, 2-(4-fluoropiperidin-1-yl)ethoxy, 2-(thiomorpholin-4-yl)ethoxy, 2-(2-methylmorpholin-4-yl)ethoxy, 2-{6-oxa-9-azaspiro[4.5]decan-9-yl}ethoxy, 2-(3,3-difluoropyrrolidin-1-yl)ethoxy, 2-{4-oxa-7-azaspiro[2.5]octan-7-yl}ethoxy, 2,6-dimethylmorpholin-4-yl]ethoxy, 2-[cyclopropyl(methyl)amino]ethoxy, 2-{methyl[(oxetan-3-yl)methyl]amino}ethoxy, 2-[methyl(oxetan-3-yl)amino]ethoxy, 2-(4-fluoropiperidin-1-yl)ethoxy, 2-[(2-fluoroethyl)(methyl)amino]ethoxy, 2-[4-(2-fluoroethyl)piperazin-1-yl]ethoxy, 2-(4-methylpiperazin-1-yl)ethoxy, 2-(2,2-dimethylmorpholin-4-yl)ethoxy, 2-(morpholin-4-yl)propoxy, 2-(4,4-difluoropiperidin-1-yl)ethyl, [2-methyl-1-(morpholin-4-yl)propan-2-yl]oxy, 2-(3,3-dimethylmorpholin-4-yl)ethoxy, and [(oxan-4-yl)methoxy]methyl;
(x) the group:

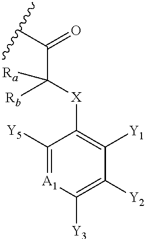

represents

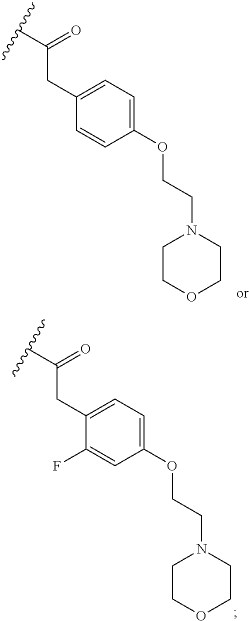 [SMILES: *C(=O)Cc1ccc(OCCN2CCOCC2)cc1 ; *C(=O)Cc1ccc(OCCN2CCOCC2)cc1F]

(xi) T represents a linear or branched (C₁-C₆)alkyl group or a (C₁-C₄)alkylene-NR₁R₂ group; and/or
(xii) T represents a group selected from: methyl, (piperidin-1-yl)methyl, (morpholin-4-yl)methyl, [(3R)-3-fluoropyrrolidin-1-yl]methyl, [methyl(propan-2-yl)amino]methyl, (azepan-1-yl)methyl, (pyrrolidin-1-yl)methyl, [(3S)-3-methylpiperidin-1-yl]methyl, [(3R)-3-methylpiperidin-1-yl]methyl, [(1RS,5SR)-3-azabicyclo[3.1.0]hexan-3-yl]methyl, [(2S)-2-methylpiperidin-1-yl]methyl, {6-azaspiro[2.5]octan-6-yl}methyl, (4,4-difluoropiperidin-1-yl)methyl, (4-methylpiperidin-1-yl)methyl, [ethyl(propan-2-yl)amino]methyl, (3R)-3-methylpyrrolidin-1-yl]methyl, and (3S)-3-{[(3S)-3-methylpyrrolidin-1-yl]methyl.

In some embodiments, in Formula (V), (Va), (Vb), (Vc), (Vd), (Ve), (Vf), (Vg), (Vh), (Vi) or (Vj), R₅ represents a hydroxy group and R₆ represents a hydrogen atom. In some embodiments, in Formula (V), (Va), (Vb), (Vc), (Vd), (Ve), (Vf), (Vg), (Vh), (Vi) or (Vj), Y₃ represents a —O—(C₁-C₄)alkylene-Cy₃ group.

In an ninetieth embodiment, the present disclosure provides an antibody-drug conjugate of the seventy-sixth embodiment, wherein the Bcl-2 inhibitor is represented by any one of the following or a pharmaceutically acceptable salt thereof:

TABLE A3

| | |
|---|---|
| 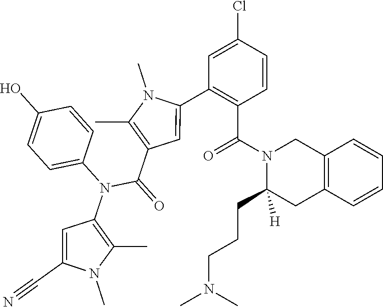 [SMILES: Cc1c(N(C(=O)c2cc(-c3cc(Cl)ccc3C(=O)N3Cc4ccccc4C[C@H]3CCCN(C)C)n(C)c2C)c2ccc(O)cc2)cc(C#N)n1C] | D3-1 |
| 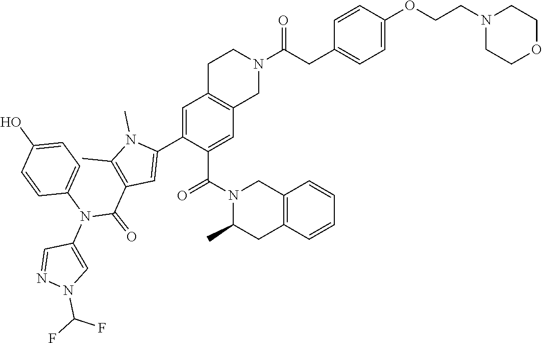 [SMILES: Cc1c(C(=O)N(c2ccc(O)cc2)c2cnn(C(F)F)c2)cc(-c2cc3c(cc2C(=O)N2Cc4ccccc4C[C@H]2C)CN(C(=O)Cc2ccc(OCCN4CCOCC4)cc2)CC3)n1C] | D3-2 |
| 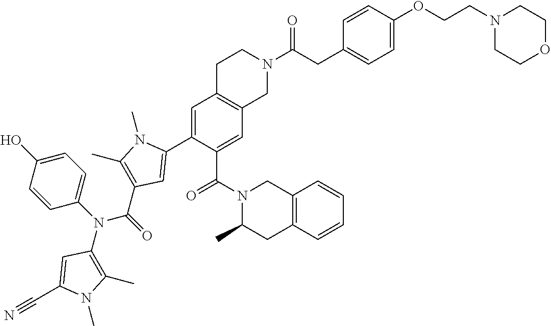 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(O)cc2)cc(C#N)n1C] | D3-3 |
| 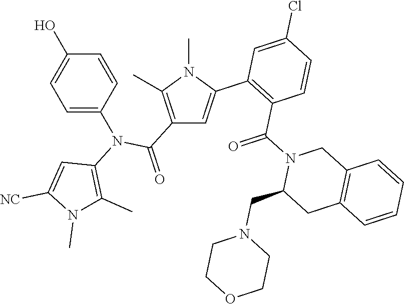 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C] | D3-4 |
| 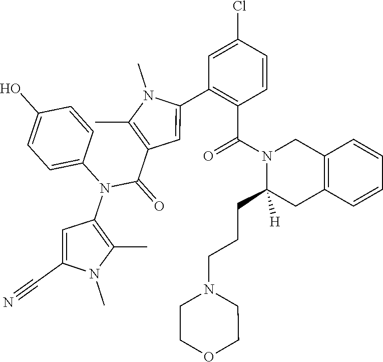 [SMILES: Cc1c(N(C(=O)c2cc(-c3cc(Cl)ccc3C(=O)N3Cc4ccccc4C[C@H]3CCCN3CCOCC3)n(C)c2C)c2ccc(O)cc2)cc(C#N)n1C] | D3-5 |
| 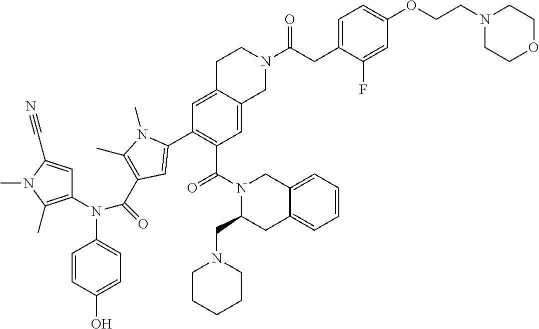 [SMILES: Cc1c(N(C(=O)c2cc(-c3cc4c(cc3C(=O)N3Cc5ccccc5C[C@H]3CN3CCCCC3)CN(C(=O)Cc3ccc(OCCN5CCOCC5)cc3F)CC4)n(C)c2C)c2ccc(O)cc2)cc(C#N)n1C] | D3-6 |
| 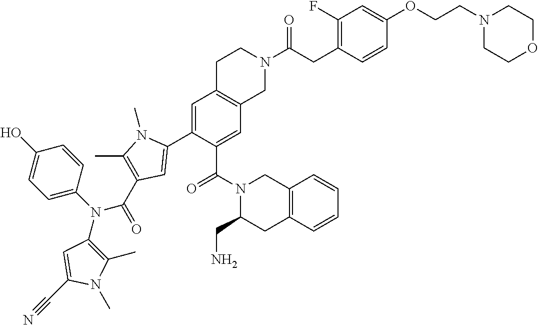 [SMILES: Cc1c(N(C(=O)c2cc(-c3cc4c(cc3C(=O)N3Cc5ccccc5C[C@H]3CN)CN(C(=O)Cc3ccc(OCCN5CCOCC5)cc3F)CC4)n(C)c2C)c2ccc(O)cc2)cc(C#N)n1C] | D3-7 |
| 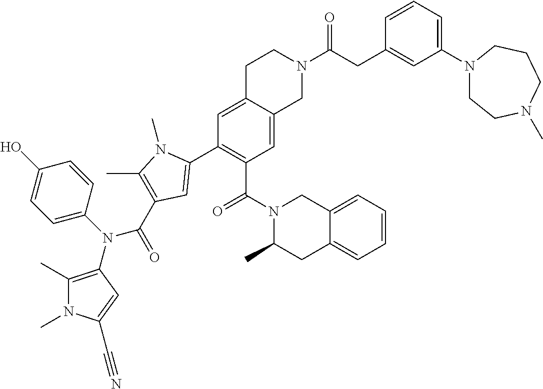 [SMILES: Cc1c(N(C(=O)c2cc(-c3cc4c(cc3C(=O)N3Cc5ccccc5C[C@H]3C)CN(C(=O)Cc3cccc(N5CCCN(C)CC5)c3)CC4)n(C)c2C)c2ccc(O)cc2)cc(C#N)n1C] | D3-8 |
| 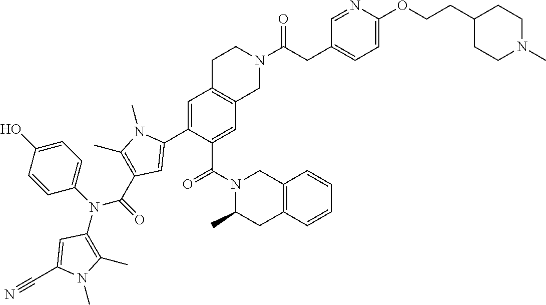 [SMILES: Cc1c(N(C(=O)c2cc(-c3cc4c(cc3C(=O)N3Cc5ccccc5C[C@H]3C)CN(C(=O)Cc3ccc(OCCC5CCN(C)CC5)nc3)CC4)n(C)c2C)c2ccc(O)cc2)cc(C#N)n1C] | D3-9 |
| 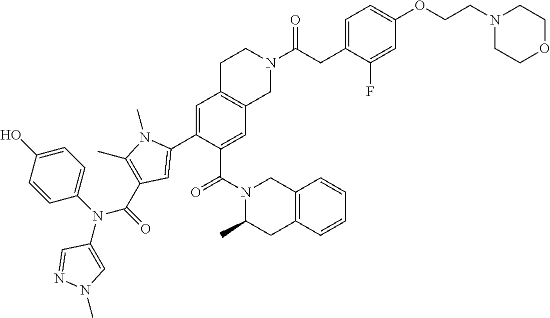 [SMILES: Cc1c(C(=O)N(c2ccc(O)cc2)c2cnn(C)c2)cc(-c2cc3c(cc2C(=O)N2Cc4ccccc4C[C@H]2C)CN(C(=O)Cc2ccc(OCCN4CCOCC4)cc2F)CC3)n1C] | D3-10 |
| 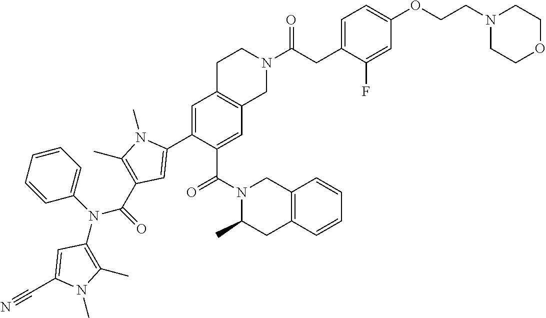 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3F)CC4)n(C)c2C)c2ccccc2)cc(C#N)n1C] | D3-11 |
| 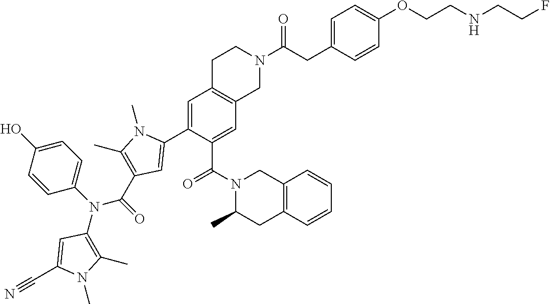 [SMILES: Cc1c(N(C(=O)c2cc(-c3cc4c(cc3C(=O)N3Cc5ccccc5C[C@H]3C)CN(C(=O)Cc3ccc(OCCNCCF)cc3)CC4)n(C)c2C)c2ccc(O)cc2)cc(C#N)n1C] | D3-12 |
| 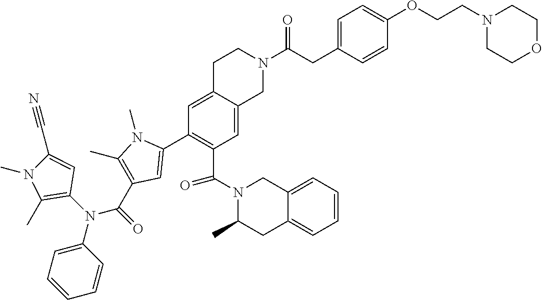 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cc(C#N)n1C] | D3-13 |
| 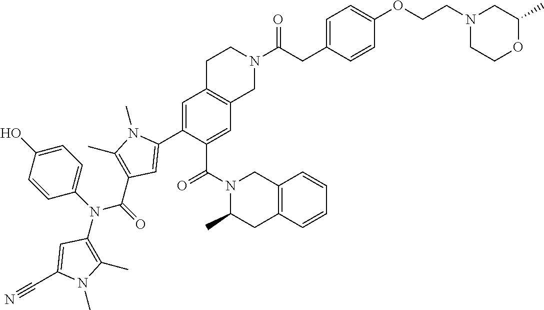 [SMILES: Cc1c(N(C(=O)c2cc(-c3cc4c(cc3C(=O)N3Cc5ccccc5C[C@H]3C)CN(C(=O)Cc3ccc(OCCN5CCO[C@@H](C)C5)cc3)CC4)n(C)c2C)c2ccc(O)cc2)cc(C#N)n1C] | D3-14 |
| 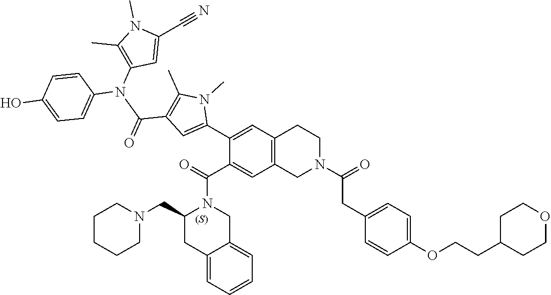 | D3-15 |
| 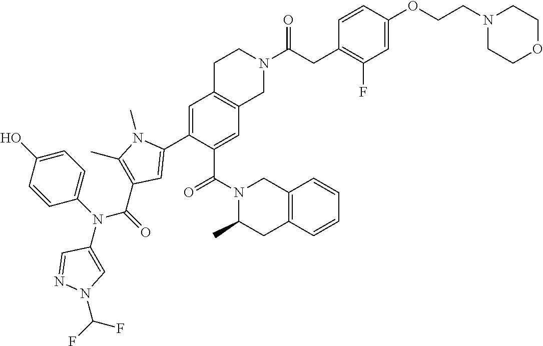 [SMILES: Cc1c(C(=O)N(c2ccc(O)cc2)c2cnn(C(F)F)c2)cc(-c2cc3c(cc2C(=O)N2Cc4ccccc4C[C@H]2C)CN(C(=O)Cc2ccc(OCCN4CCOCC4)cc2F)CC3)n1C] | D3-16 |
| 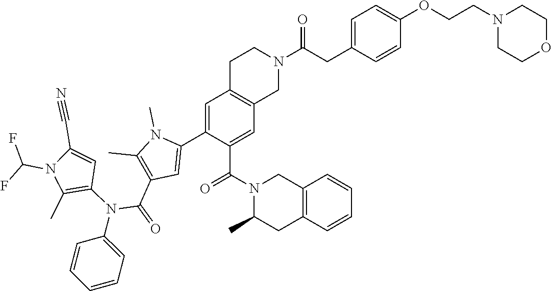 [SMILES: Cc1c(C(=O)N(c2ccccc2)c2cc(C#N)n(C(F)F)c2C)cc(-c2cc3c(cc2C(=O)N2Cc4ccccc4C[C@H]2C)CN(C(=O)Cc2ccc(OCCN4CCOCC4)cc2)CC3)n1C] | D3-17 |

The definitions of the remaining variables are provided in the seventy-sixth embodiment or any embodiments described therein.

In some embodiments, the present disclosure provides an antibody-drug conjugate described in any one of the first to forty-sixth, forty-ninth, fifty-first to ninieth embodiments, wherein the topoisomerase 1 inhibitor is represented by any one of the following or a pharmaceutically acceptable salt thereof:

TABLE A4

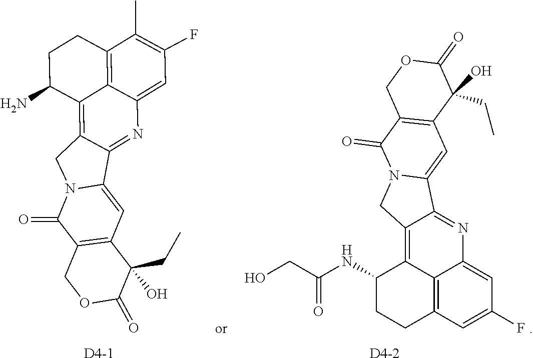

The definitions of the remaining variables are provided in any one of the first to forty-sixth, forty-ninth, fifty-first to ninieth embodiments or any embodiments described therein.

In some embodiments, the present disclosure provides an antibody-drug conjugate described in any one of the first to forty-sixth, forty-ninth, fifty-first to ninieth embodiments, wherein anti-mitotic drug monomethyl auristatin E (MMAE) or a taxane. The definitions of the remaining variables are provided in any one of the first to forty-sixth, forty-ninth, fifty-first to ninieth embodiments or any embodiments described therein. In some embodiments, the taxane is selected from docetaxel, paclitaxel, or cabazitaxel.

In a ninety-first embodiment, the present disclosure provides an antibody-drug conjugate of any one of the first through ninetieth embodiments, wherein the antibody or antigen-binding fragment binds to a target antigen on a cancer cell. The definitions of the remaining variables are provided in the first through ninetieth embodiments or any embodiments described therein.

In a ninety-second embodiment, the present disclosure provides an antibody-drug conjugate of the ninety-first embodiment, wherein:
(i) the target antigen is selected from BCMA, CD33, HER2, CD38, CD48, CD79b, PCAD, CD74, CD138, SLAMF7, CD123, CLL1, FLT3, CD7, CKIT, CD56, SEZ6, DLL3, DLK1, B7-H3, EGFR, CD71, EphA2, EPCAM, FOLR1, ENPP3, MET, AXL, SLC34A2, Nectin4, TROP2, LIV1, CD46, MSLN, F3, MUC16, SLC39A6, TFRC, TACSTD2, and GPNMB;
(ii) the target antigen is selected from EGFR, CD7, HER2, EPCAM, FOLR1, ENPP3, MET, AXL, SLC34A2, Nectin4, MSLN, F3, MUC16, SLC39A6, TFRC, TACSTD2, and GPNMB; or (iii) the target antigen is MET, CD48, CD74, EphA2, PCAD, TROP2, B7-H3, or 5T4 or HER2.

The definitions of the remaining variables are provided in the ninety-first embodiment.

In a ninety-third embodiment, the present disclosure provides an antibody-drug conjugate of the ninety-first embodiment, wherein the antibody or antigen-binding fragment having CDR sequences is selected from those in Tables D3 and D8, or the antibody or antigen-binding fragment having variable regions is selected from those in Tables D2 and D8, or the antibody or antigen-binding fragment having full length is selected from those in Tables D4, D5, and D7. The definitions of the remaining variables are provided in the ninety-first embodiment.

In a ninety-fourth embodiment, the present disclosure provides an antibody-drug conjugate of the ninety-first embodiment, wherein the antibody or antigen-binding fragment thereof is an anti-CD74 antibody comprising three heavy chain CDRs and three light chain CDRs selected from the group consisting of:
1) heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:268, light chain CDR2 (LCDR2) consisting of SEQ ID NO:264, and light chain CDR3 (LCDR3) consisting of SEQ ID NO:265;
2) heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174;
3) heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:269, light chain CDR2 (LCDR2) consisting of SEQ ID NO:264, and light chain CDR3 (LCDR3) consisting of SEQ ID NO:174;
4) heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174;
5) heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:263, light chain CDR2 (LCDR2) consisting of SEQ ID NO:264, and light chain CDR3 (LCDR3) consisting of SEQ ID NO:265;
6) heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174;
7) heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:267, light chain CDR2 (LCDR2) consisting of SEQ ID NO:264, and light chain CDR3 (LCDR3) consisting of SEQ ID NO:174; and
8) heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174.

The definitions of the remaining variables are provided in the ninety-first embodiment.

In a ninety-fifth embodiment, the present disclosure provides an antibody-drug conjugate of the ninety-first embodiment, wherein the antibody or antigen-binding fragment thereof is an anti-CD74 antibody comprising (a) a heavy chain variable region comprising the amino acid sequence of SEQ ID NO:153, and a light chain variable region comprising the amino acid sequence of SEQ ID NO:262, or (b) a heavy chain variable region comprising the amino acid sequence of SEQ ID NO:153, and a light chain variable region comprising the amino acid sequence of SEQ ID NO:267. The definitions of the remaining variables are provided in the ninety-first embodiment.

In a ninety-sixth embodiment, the present disclosure provides an antibody-drug conjugate of the ninety-first embodiment, wherein the antibody or antigen-binding fragment thereof is an anti-CD74 antibody comprising:
(a) the heavy chain amino acid sequence of SEQ ID NO:118 or a sequence that is at least 95% identical to SEQ ID NO:118, and the light chain amino acid sequence of SEQ ID NO:237 or a sequence that is at least 95% identical to SEQ ID NO:237;
(b) the heavy chain amino acid sequence of SEQ ID NO:236 or a sequence that is at least 95% identical to SEQ ID NO:236, and the light chain amino acid sequence of SEQ ID NO:237 or a sequence that is at least 95% identical to SEQ ID NO:237; or
(c) the heavy chain amino acid sequence of SEQ ID NO:118 or a sequence that is at least 95% identical to SEQ ID NO:118, and the light chain amino acid sequence of SEQ ID NO:239 or a sequence that is at least 95% identical to SEQ ID NO:239.

The definitions of the remaining variables are provided in the ninety-first embodiment.

In a ninety-seventh embodiment, the present disclosure provides an antibody-drug conjugate of the ninety-first embodiment, wherein the antibody or antigen-binding fragment thereof is an anti-CD48 antibody comprising three heavy chain CDRs and three light chain CDRs selected from the group consisting of:
1) heavy chain CDR1 (HCDR1) consisting of SEQ ID NO:271, heavy chain CDR2 (HCDR2) consisting of SEQ ID NO:272, heavy chain CDR3 (HCDR3) consisting of SEQ ID NO:273; light chain CDR1 (LCDR1) consisting of SEQ ID NO:281, light chain CDR2 (LCDR2) consisting of SEQ ID NO:282, and light chain CDR3 (LCDR3) consisting of SEQ ID NO:283;
2) heavy chain CDR1 (HCDR1) consisting of SEQ ID NO:274, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:285, and light chain CDR3 (LCDR3) consisting of SEQ ID NO:286;
3) heavy chain CDR1 (HCDR1) consisting of SEQ ID NO:276, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:287, light chain CDR2 (LCDR2) consisting of SEQ ID NO:282, and light chain CDR3 (LCDR3) consisting of SEQ ID NO:286;
4) heavy chain CDR1 (HCDR1) consisting of SEQ ID NO:279, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:288, and light chain CDR3 (LCDR3) consisting of SEQ ID NO:286; and
5) heavy chain CDR1 (HCDR1) consisting of SEQ ID NO:51, heavy chain CDR2 (HCDR2) consisting of SEQ ID NO:52, heavy chain CDR3 (HCDR3) consisting of SEQ ID NO:53; light chain CDR1 (LCDR1) consisting of SEQ ID NO:54, light chain CDR2 (LCDR2) consisting of SEQ ID NO:55, and light chain CDR3 (LCDR3) consisting of SEQ ID NO:56.

The definitions of the remaining variables are provided in the ninety-first embodiment.

In a ninety-eighth embodiment, the present disclosure provides an antibody-drug conjugate of the ninety-first embodiment, wherein the antibody or antigen-binding fragment thereof is an anti-CD48 antibody comprising a) a heavy chain variable region comprising the amino acid sequence of SEQ ID NO:270, and a light chain variable region comprising the amino acid sequence of SEQ ID NO:280; or b) a heavy chain variable region comprising the amino acid sequence of SEQ ID NO:13, and a light chain variable region comprising the amino acid sequence of SEQ ID NO:14. The definitions of the remaining variables are provided in the ninety-first embodiment.

In a ninety-ninth embodiment, the present disclosure provides an antibody-drug conjugate of the ninety-first embodiment, wherein the antibody or antigen-binding fragment thereof is an anti-CD48 antibody comprising (a) the heavy chain amino acid sequence of SEQ ID NO:240 or a sequence that is at least 95% identical to SEQ ID NO:240, and the light chain amino acid sequence of SEQ ID NO:243 or a sequence that is at least 95% identical to SEQ ID NO:243; or (b) the heavy chain amino acid sequence of SEQ ID NO:242 or a sequence that is at least 95% identical to SEQ ID NO:242, and the light chain amino acid sequence of SEQ ID NO:243 or a sequence that is at least 95% identical to SEQ ID NO:243; c) the heavy chain amino acid sequence of SEQ ID NO:240 or a sequence that is at least 95% identical to SEQ ID NO:240, and the light chain amino acid sequence of SEQ ID NO:69 or a sequence that is at least 95% identical to SEQ ID NO:70. The definitions of the remaining variables are provided in the ninety-first embodiment.

In a one hundredth embodiment, the present disclosure provides an antibody-drug conjugate of the ninety-first embodiment,, wherein the antibody or antigen-binding fragment thereof is an anti-Her2 antibody comprising three heavy chain CDRs and three light chain CDRs selected from the group consisting of:
1) heavy chain CDR1 (HCDR1) consisting of SEQ ID NO:289, heavy chain CDR2 (HCDR2) consisting of SEQ ID NO:290, heavy chain CDR3 (HCDR3) consisting of SEQ ID NO:291; light chain CDR1 (LCDR1) consisting of SEQ ID NO:297, light chain CDR2 (LCDR2) consisting of SEQ ID NO:298, and light chain CDR3 (LCDR3) consisting of SEQ ID NO:299;
2) heavy chain CDR1 (HCDR1) consisting of SEQ ID NO:292, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44;
3) heavy chain CDR1 (HCDR1) consisting of SEQ ID NO:293, heavy chain CDR2 (HCDR2) consisting of SEQ ID NO:294, heavy chain CDR3 (HCDR3) consisting of SEQ ID NO:295; light chain CDR1 (LCDR1) consisting of SEQ ID NO:302, light chain CDR2 (LCDR2) consisting of SEQ ID NO:298, and light chain CDR3 (LCDR3) consisting of SEQ ID NO:44; and
4) heavy chain CDR1 (HCDR1) consisting of SEQ ID NO:39, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44.

The definitions of the remaining variables are provided in the ninety-first embodiment.

In a one hundred and first embodiment, the present disclosure provides an antibody-drug conjugate of the ninety-first embodiment, wherein the antibody or antigen-binding fragment thereof is an anti-Her2 antibody comprising a heavy chain variable region comprising the amino acid sequence of SEQ ID NO:9, and a light chain variable region comprising the amino acid sequence of SEQ ID NO:296. The definitions of the remaining variables are provided in the ninety-first embodiment.

In a one hundred and second embodiment, the present disclosure provides an antibody-drug conjugate of the ninety-first embodiment, wherein the antibody or antigen-binding fragment thereof is an anti-Her2 antibody comprising the heavy chain amino acid sequence of SEQ ID NO:245 or a sequence that is at least 95% identical to SEQ ID NO:245, and the light chain amino acid sequence of SEQ ID NO:66 or a sequence that is at least 95% identical to SEQ ID NO:66. The definitions of the remaining variables are provided in the ninety-first embodiment.

In a one hundred and third embodiment, the present disclosure provides an antibody-drug conjugate of the ninety-first embodiment, wherein the antibody or antigen-binding fragment thereof is an anti-PCAD antibody comprising three heavy chain CDRs and three light chain CDRs selected from the group consisting of:
1) heavy chain CDR1 (HCDR1) consisting of SEQ ID NO:304, heavy chain CDR2 (HCDR2) consisting of SEQ ID NO:305, heavy chain CDR3 (HCDR3) consisting of SEQ ID NO:306; light chain CDR1 (LCDR1) consisting of SEQ ID NO:312, light chain CDR2 (LCDR2) consisting of SEQ ID NO:313, and light chain CDR3 (LCDR3) consisting of SEQ ID NO:314;
2) heavy chain CDR1 (HCDR1) consisting of SEQ ID NO:307, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316;
3) heavy chain CDR1 (HCDR1) consisting of SEQ ID NO:309, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:317, light chain CDR2 (LCDR2) consisting of SEQ ID NO:313, and light chain CDR3 (LCDR3) consisting of SEQ ID NO:316; and
4) heavy chain CDR1 (HCDR1) consisting of SEQ ID NO:310, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316.

The definitions of the remaining variables are provided in the ninety-first embodiment.

In a one hundred and fourth embodiment, the present disclosure provides an antibody-drug conjugate of the ninety-first embodiment, wherein the antibody or antigen-binding fragment thereof is an anti-PCAD antibody comprising a heavy chain variable region comprising the amino acid sequence of SEQ ID NO:303, and a light chain variable region comprising the amino acid sequence of SEQ ID NO:311. The definitions of the remaining variables are provided in the ninety-first embodiment.

In a one hundred and fifth embodiment, the present disclosure provides an antibody-drug conjugate of the ninety-first embodiment, wherein the antibody or antigen-binding fragment thereof is an anti-PCAD antibody comprising the heavy chain amino acid sequence of SEQ ID NO:248 or a sequence that is at least 95% identical to SEQ ID NO:248, and the light chain amino acid sequence of SEQ ID NO:250 or a sequence that is at least 95% identical to SEQ ID NO:250. The definitions of the remaining variables are provided in the ninety-first embodiment.

In a one hundred and sixth embodiment, the present disclosure provides an antibody-drug conjugate of the ninety-first embodiment, wherein the antibody or antigen-binding fragment thereof is an anti-EphA2 antibody comprising three heavy chain CDRs and three light chain CDRs selected from the group consisting of:
1) heavy chain CDR1 (HCDR1) consisting of SEQ ID NO:319, heavy chain CDR2 (HCDR2) consisting of SEQ ID NO:320, heavy chain CDR3 (HCDR3) consisting of SEQ ID NO:321; light chain CDR1 (LCDR1) consisting of SEQ ID NO:330, light chain CDR2 (LCDR2) consisting of SEQ ID NO:331, and light chain CDR3 (LCDR3) consisting of SEQ ID NO:332;
2) heavy chain CDR1 (HCDR1) consisting of SEQ ID NO:322, heavy chain CDR2 (HCDR2) consisting of SEQ ID NO:323, heavy chain CDR3 (HCDR3) consisting of SEQ ID NO:324; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335;
3) heavy chain CDR1 (HCDR1) consisting of SEQ ID NO:325, heavy chain CDR2 (HCDR2) consisting of SEQ ID NO:326, heavy chain CDR3 (HCDR3) consisting of SEQ ID NO:327; light chain CDR1 (LCDR1) consisting of SEQ ID NO:336, light chain CDR2 (LCDR2) consisting of SEQ ID NO:331, and light chain CDR3 (LCDR3) consisting of SEQ ID NO:335; and
4) heavy chain CDR1 (HCDR1) consisting of SEQ ID NO:328, heavy chain CDR2 (HCDR2) consisting of SEQ ID NO:323, heavy chain CDR3 (HCDR3) consisting of SEQ ID NO:321; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335.

The definitions of the remaining variables are provided in the ninety-first embodiment.

In a one hundred and seventh embodiment, the present disclosure provides an antibody-drug conjugate of the ninety-first embodiment, wherein the antibody or antigen-binding fragment thereof is an anti-EphA2 antibody comprising a heavy chain variable region comprising the amino acid sequence of SEQ ID NO:318, and a light chain variable region comprising the amino acid sequence of SEQ ID NO:329. The definitions of the remaining variables are provided in the ninety-first embodiment.

In a one hundred and eighth embodiment, the present disclosure provides an antibody-drug conjugate of the ninety-first embodiment, wherein the antibody or antigen-binding fragment thereof is an anti-EphA2 antibody comprising the heavy chain amino acid sequence of SEQ ID NO:252 or a sequence that is at least 95% identical to SEQ ID NO:252, and the light chain amino acid sequence of SEQ ID NO:254 or a sequence that is at least 95% identical to SEQ ID NO:254. The definitions of the remaining variables are provided in the ninety-first embodiment.

In a one hundred and ninth embodiment, the present disclosure provides an antibody-drug conjugate of the ninety-first embodiment, wherein the antibody or antigen-binding fragment thereof is an anti-MET antibody comprising three heavy chain CDRs and three light chain CDRs selected from the group consisting of:
1) heavy chain CDR1 (HCDR1) consisting of SEQ ID NO:349, heavy chain CDR2 (HCDR2) consisting of SEQ ID NO:350, heavy chain CDR3 (HCDR3) consisting of SEQ ID NO:351; light chain CDR1 (LCDR1) consisting of SEQ ID NO:352, light chain CDR2 (LCDR2) consisting of SEQ ID NO:353, and light chain CDR3 (LCDR3) consisting of SEQ ID NO: 354;
2) heavy chain CDR1 (HCDR1) consisting of SEQ ID NO:355, heavy chain CDR2 (HCDR2) consisting of SEQ ID NO:356, heavy chain CDR3 (HCDR3) consisting of SEQ ID NO:357; light chain CDR1 (LCDR1) consisting of SEQ ID NO:358, light chain CDR2 (LCDR2) consisting of SEQ ID NO:359, and light chain CDR3 (LCDR3) consisting of SEQ ID NO: 360; and
3) heavy chain CDR1 (HCDR1) consisting of SEQ ID NO:361, heavy chain CDR2 (HCDR2) consisting of SEQ ID NO:362, heavy chain CDR3 (HCDR3) consisting of SEQ ID NO:363; light chain CDR1 (LCDR1) consisting of SEQ ID NO:364, light chain CDR2 (LCDR2) consisting of SEQ ID NO:365, and light chain CDR3 (LCDR3) consisting of SEQ ID NO: 366. The definitions of the remaining variables are provided in the ninety-first embodiment.

In a one hundred and tenth embodiment, the present disclosure provides an antibody-drug conjugate of the ninety-first embodiment, wherein the antibody or antigen-binding fragment thereof is an anti- MET antibody comprising a heavy chain variable region and a light chain variable region selected from the group consisting of:
1) a heavy chain variable region comprising the amino acid sequence of SEQ ID NO:339, and a light chain variable region comprising the amino acid sequence of SEQ ID NO:340;
2) a heavy chain variable region comprising the amino acid sequence of SEQ ID NO:341, and a light chain variable region comprising the amino acid sequence of SEQ ID NO:342; and
3) a heavy chain variable region comprising the amino acid sequence of SEQ ID NO:343, and a light chain variable region comprising the amino acid sequence of SEQ ID NO:344.

The definitions of the remaining variables are provided in the ninety-first embodiment.

In a one hundred and eleventh embodiment, the present disclosure provides an antibody-drug conjugate of the ninety-first embodiment, wherein the antibody or antigen-binding fragment thereof is an anti- MET antibody comprising a heavy chain variable region and a light chain variable region selected from the group consisting of:
1) the heavy chain amino acid sequence of SEQ ID NO:367 or a sequence that is at least 95% identical to SEQ ID NO:367, and the light chain amino acid sequence of SEQ ID NO:368 or a sequence that is at least 95% identical to SEQ ID NO:368;
2) the heavy chain amino acid sequence of SEQ ID NO:369 or a sequence that is at least 95% identical to SEQ ID NO:369, and the light chain amino acid sequence of SEQ ID NO:370 or a sequence that is at least 95% identical to SEQ ID NO:370;
3) the heavy chain amino acid sequence of SEQ ID NO:371 or a sequence that is at least 95% identical to SEQ ID NO:371, and the light chain amino acid sequence of SEQ ID NO:372 or a sequence that is at least 95% identical to SEQ ID NO:372;
4) the heavy chain amino acid sequence of SEQ ID NO:373 or a sequence that is at least 95% identical to SEQ ID NO:373, and the light chain amino acid sequence of SEQ ID NO:374 or a sequence that is at least 95% identical to SEQ ID NO:374;
5) the heavy chain amino acid sequence of SEQ ID NO:375 or a sequence that is at least 95% identical to SEQ ID NO:375, and the light chain amino acid sequence of SEQ ID NO:370 or a sequence that is at least 95% identical to SEQ ID NO:370; and
6) the heavy chain amino acid sequence of SEQ ID NO:376 or a sequence that is at least 95% identical to SEQ ID NO:376, and the light chain amino acid sequence of SEQ ID NO:372 or a sequence that is at least 95% identical to SEQ ID NO:372.

The definitions of the remaining variables are provided in the ninety-first embodiment.

In a one hundred and twelfth embodiment, the present disclosure provides an antibody-drug conjugate of any one of the one hundred ninth through one hundred eleventh embodiments, wherein the two antineoplastic payloads are Bcl-xL inhibitors.

In a one hundred and thirteenth embodiment, the present disclosure provides an antibody-drug conjugate of any one of the ninety-fourth through one hundred and twelfth embodiments, wherein the antibody or antigen binding fragment thereof comprises one or more cysteine substitutions selected from E152C, S375C, or both E152C and S375C of the heavy chain of the antibody or antigen binding fragment thereof, wherein the position is numbered according to the EU system. The definitions of the remaining variables are provided in the ninety-fourth through one hundred and twelfth embodiments.

In a one hundred and fourteenth embodiment, the present disclosure provides an antibody-drug conjugate of any one of the ninety fourth through one hundred and twelfth embodiments, wherein the antibody or antigen binding fragment thereof comprises one or more Fc silencing mutations. The definitions of the remaining variables are provided in the ninety-fourth through one hundred and twelfth embodiments.

In some embodiments, the present disclosure provides, in part, novel antibody-drug conjugate (ADC) compounds with biological activity against cancer cells. The compounds may slow, inhibit, and/or reverse tumor growth in mammals, and/or may be useful for treating human cancer patients. The present disclosure more specifically relates, in some embodiments, to ADC compounds that are capable of binding and killing cancer cells. In some embodiments, the ADC compounds disclosed herein comprise a dual linker that attaches two BH3 mimetics to a full-length antibody or an antigen-binding fragment. In some embodiments, the ADC compounds are also capable of internalizing into a target cell after binding.

In some embodiments, D¹ and/or D² in the ADC compounds disclosed herein (e.g., ADCs of Formula (A), (B), (C), (D1), (D2), or (D3) in the present disclosure) independently comprises a formula selected from any one of the formulae in Table Ala, or an enantiomer, a diastereoisomer,

and/or a pharmaceutically acceptable salt of any one of the foregoing.

TABLE Ala

| | |
|---|---|
| 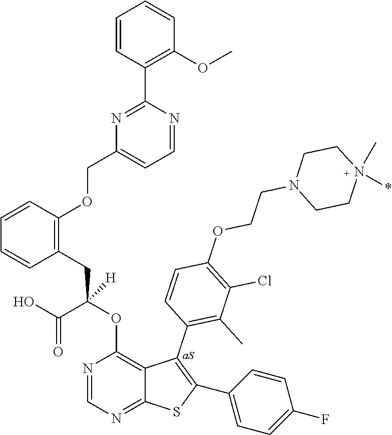 | (D1-1a) |
| 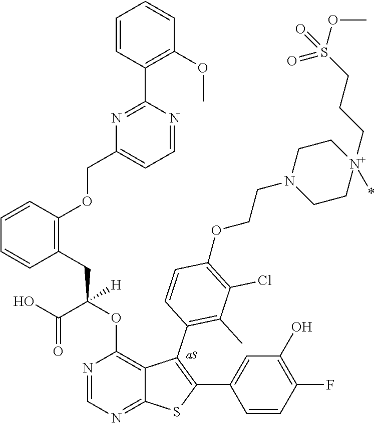 [SMILES: *[N+]1(CCCS(=O)(=O)OC)CCN(CCOc2ccc(-c3c(-c4ccc(F)c(O)c4)sc4ncnc(O[C@H](Cc5ccccc5OCc5ccnc(-c6ccccc6OC)n5)C(=O)O)c34)c(C)c2Cl)CC1] | (D1-2a) |
| 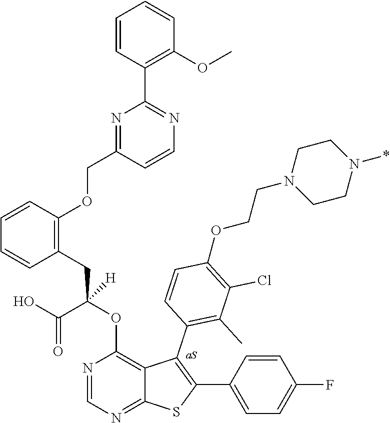 [SMILES: *N1CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1] | (D1-3a) |
| 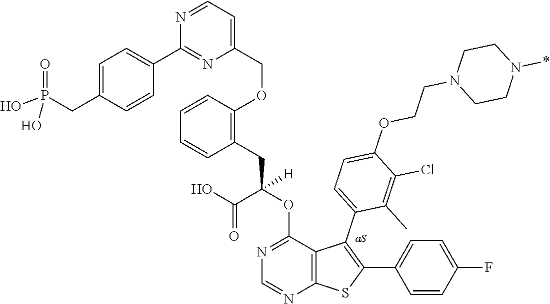 [SMILES: *N1CCN(CCOc2ccc(-c3c(-c4ccc(F)cc4)sc4ncnc(O[C@H](Cc5ccccc5OCc5ccnc(-c6ccc(CP(=O)(O)O)cc6)n5)C(=O)O)c34)c(C)c2Cl)CC1] | (D1-4a) |
| 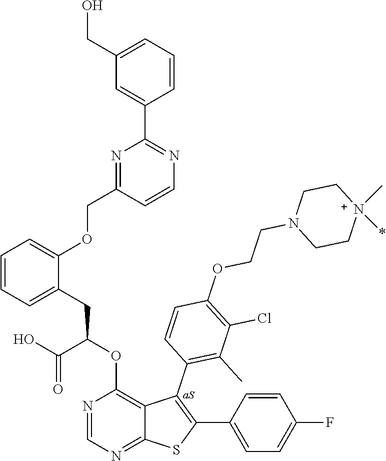 [SMILES: *[N+]1(C)CCN(CCOc2ccc(-c3c(-c4ccc(F)cc4)sc4ncnc(O[C@H](Cc5ccccc5OCc5ccnc(-c6cccc(CO)c6)n5)C(=O)O)c34)c(C)c2Cl)CC1] | (D1-5a) |
| 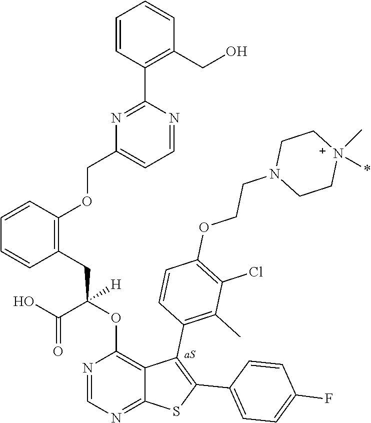 [SMILES: *[N+]1(C)CCN(CCOc2ccc(-c3c(-c4ccc(F)cc4)sc4ncnc(O[C@H](Cc5ccccc5OCc5ccnc(-c6ccccc6CO)n5)C(=O)O)c34)c(C)c2Cl)CC1] | (D1-6a) |
| 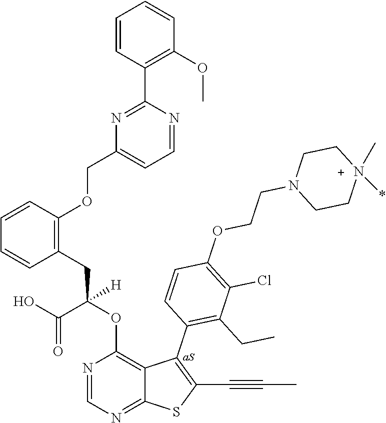 [SMILES: *[N+]1(C)CCN(CCOc2ccc(-c3c(C#CC)sc4ncnc(O[C@H](Cc5ccccc5OCc5ccnc(-c6ccccc6OC)n5)C(=O)O)c34)c(CC)c2Cl)CC1] | (D1-7a) |
| 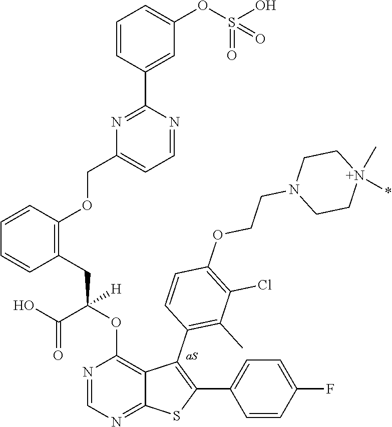 [SMILES: *[N+]1(C)CCN(CCOc2ccc(-c3c(-c4ccc(F)cc4)sc4ncnc(O[C@H](Cc5ccccc5OCc5ccnc(-c6cccc(OS(=O)(=O)O)c6)n5)C(=O)O)c34)c(C)c2Cl)CC1] | (D1-8a) |
| 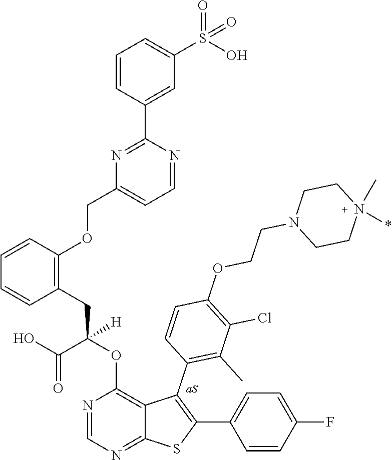 | (D1-9a) |
| 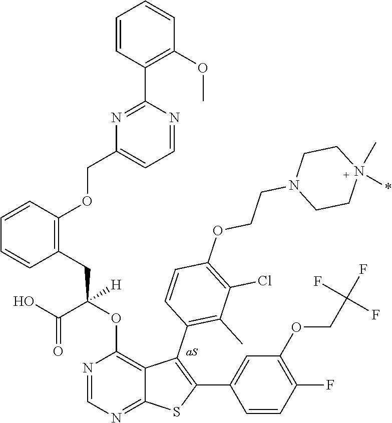 | (D1-10a) |
| 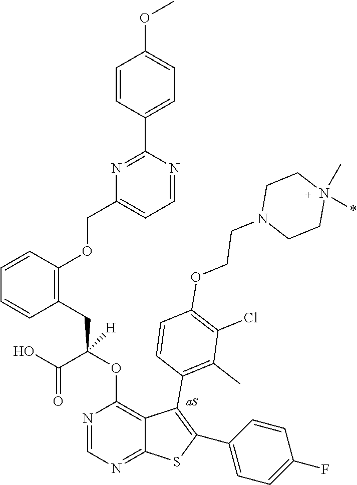 | (D1-11a) |
| 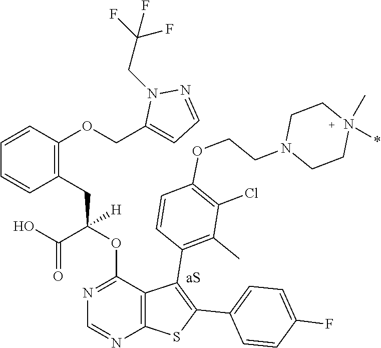 | (D1-12a) |
| 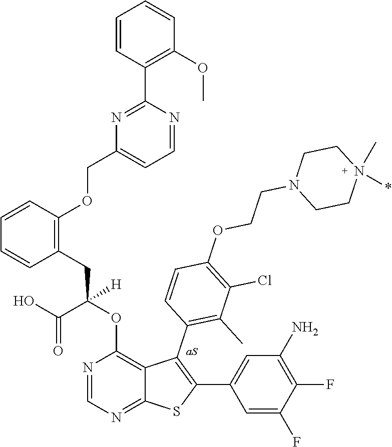 | (D1-13a) |
| 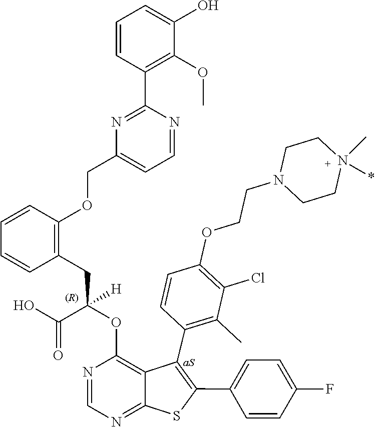 | (D1-14a) |
| 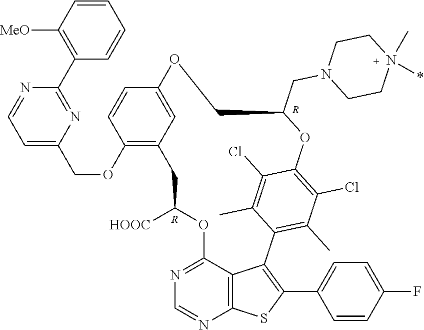 | (D1-15a) |
| 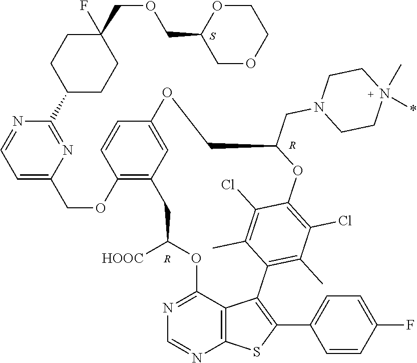 | (D1-16a) |
| 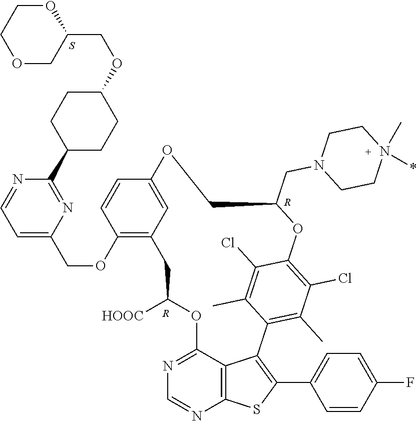 | (D1-17a) |

wherein [symbol] represents a bond to the linker.

In some embodiments, D¹ and/or D² in the ADC compounds disclosed herein (e.g., ADCs of Formula (A), (B), (C), (D1), (D2), or (D3) in the present disclosure) independently comprises a formula selected from any one of the formulae in Table A2a, or an enantiomer, a diastereoisomer, and/or a pharmaceutically acceptable salt of any one of the foregoing.

TABLE A2a

| | |
|---|---|
| 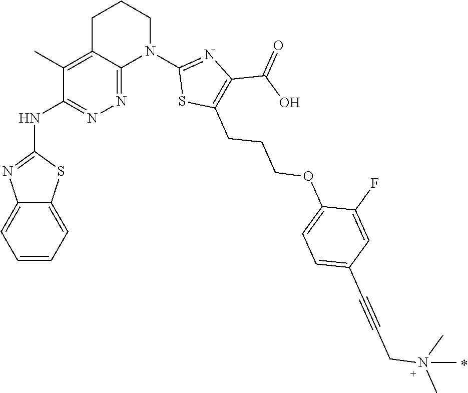 | D2-1a |
| 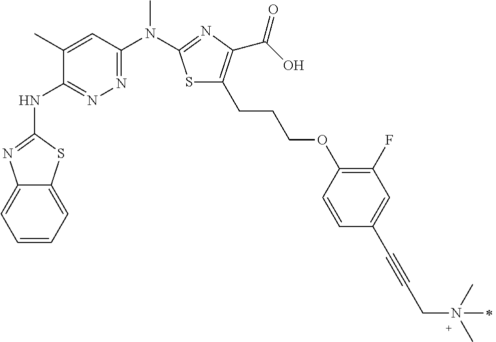 | D2-2a |
| 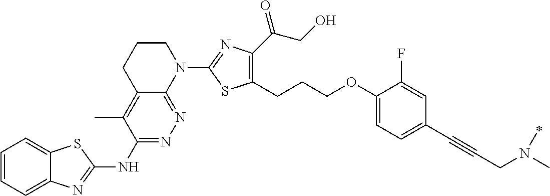 | D2-3a |
| 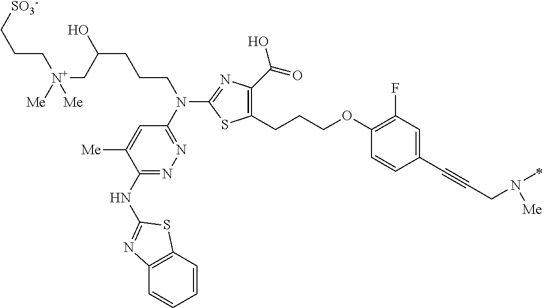 | D2-4a |
| 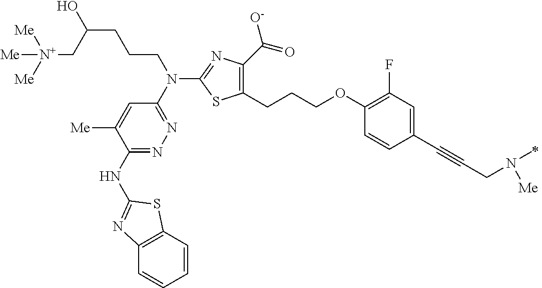 | D2-5a |
| 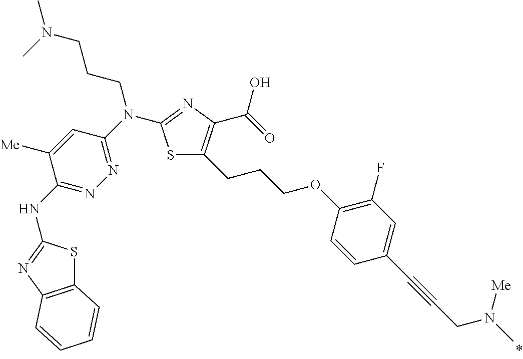 [SMILES: Cc1cc(N(CCCN(C)C)c2nc(C(=O)O)c(CCCOc3ccc(C#CCN(C)C)cc3F)s2)nnc1Nc1nc2ccccc2s1] | D2-6a |
| 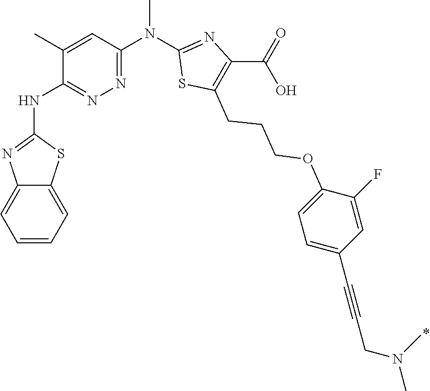 [SMILES: Cc1cc(N(C)c2nc(C(=O)O)c(CCCOc3ccc(C#CCN(C)C)cc3F)s2)nnc1Nc1nc2ccccc2s1] | D2-7a |
| 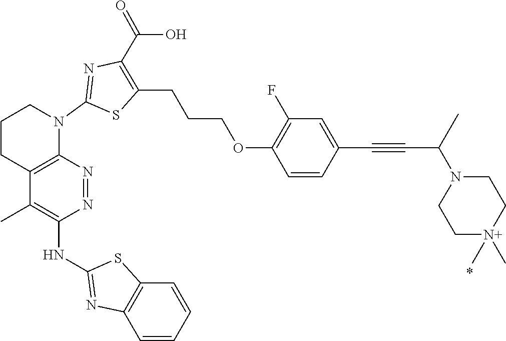 | D2-8a |
| 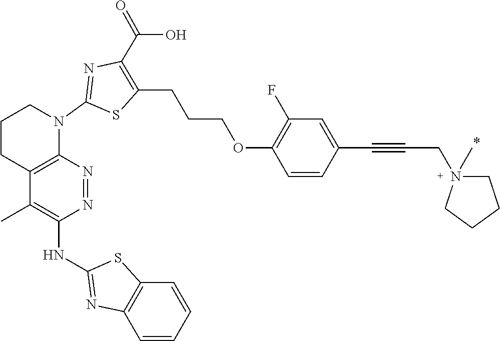 | D2-9a |
| 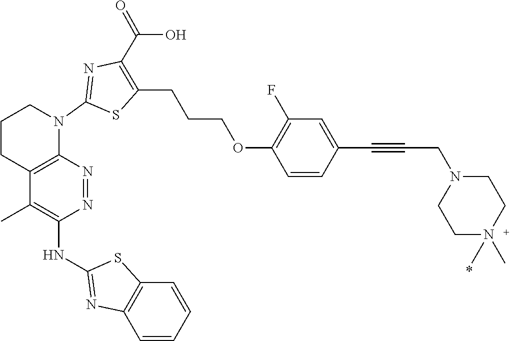 | D2-10a |
| 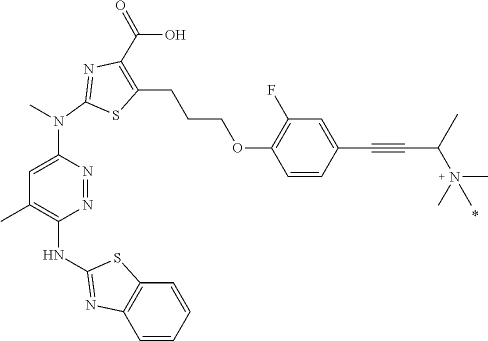 | D2-11a |
| 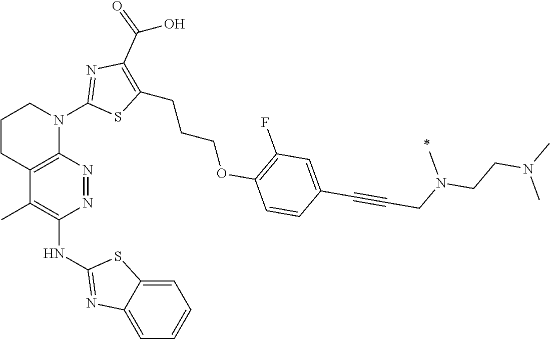 [SMILES: Cc1c(Nc2nc3ccccc3s2)nnc2c1CCCN2c1nc(C(=O)O)c(CCCOc2ccc(C#CCN(C)CCN(C)C)cc2F)s1] | D2-12a |
| 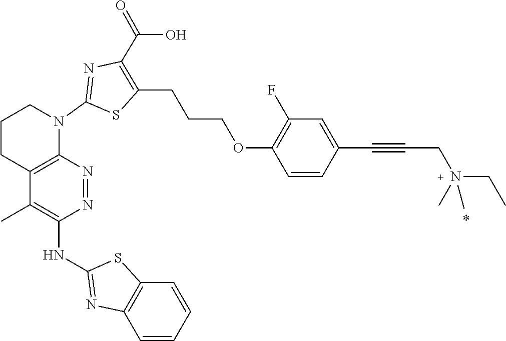 | D2-13a |
| 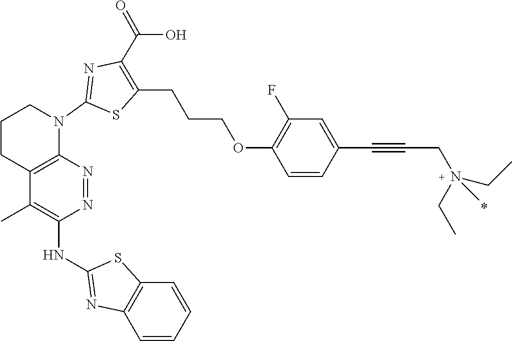 | D2-14a |
| 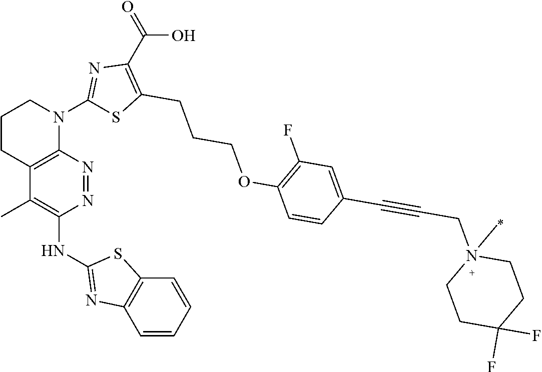 | D2-15a |
| 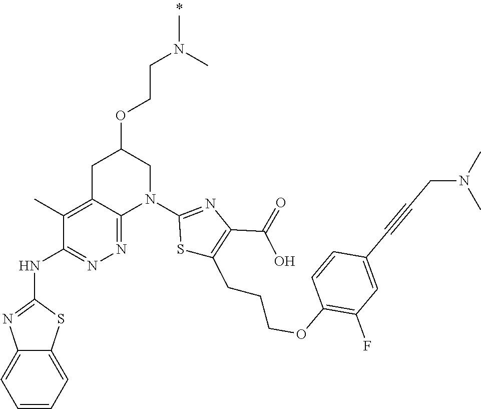 [SMILES: Cc1c(Nc2nc3ccccc3s2)nnc2c1CC(OCCN(C)C)CN2c1nc(C(=O)O)c(CCCOc2ccc(C#CCN(C)C)cc2F)s1] | D2-16a |
| 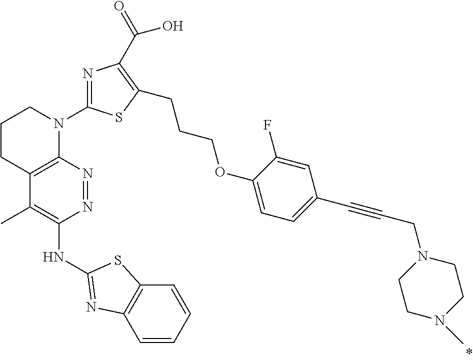 [SMILES: Cc1c(Nc2nc3ccccc3s2)nnc2c1CCCN2c1nc(C(=O)O)c(CCCOc2ccc(C#CCN3CCN(C)CC3)cc2F)s1] | D2-17a |
| 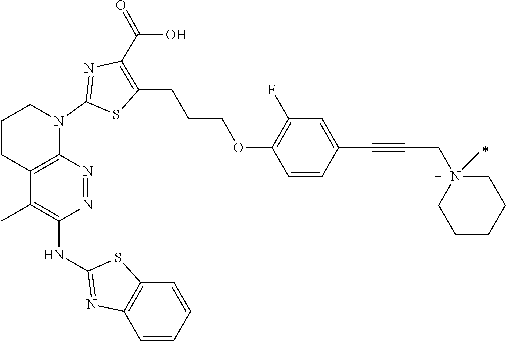 | D2-18a |
| 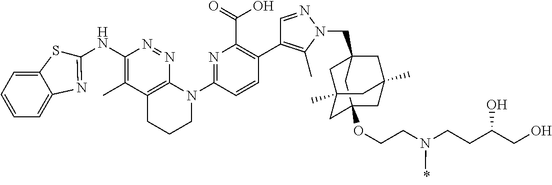 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)CC[C@H](O)CO)(C5)C4)c2C)c(C(=O)O)n1] | D2-19a |
| 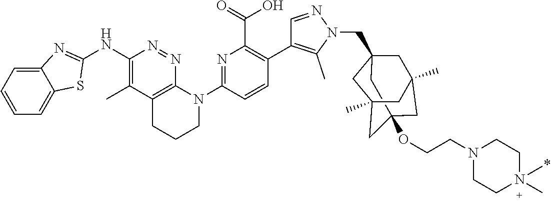 | D2-20a |
| 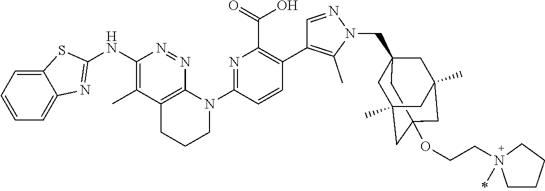 | D2-21a |
| 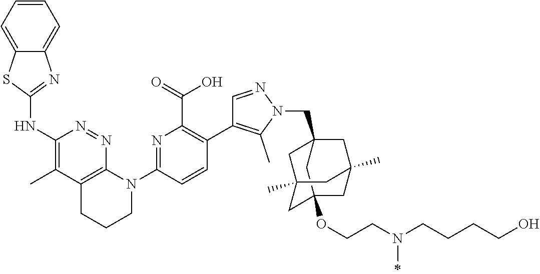 | D2-22a |
| 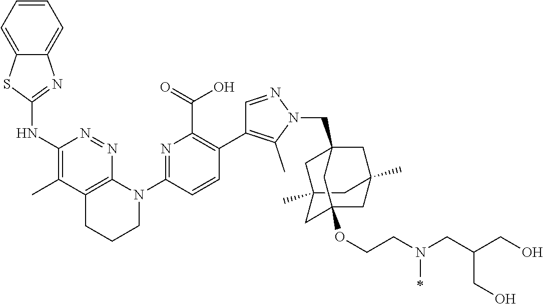 | D2-23a |
| 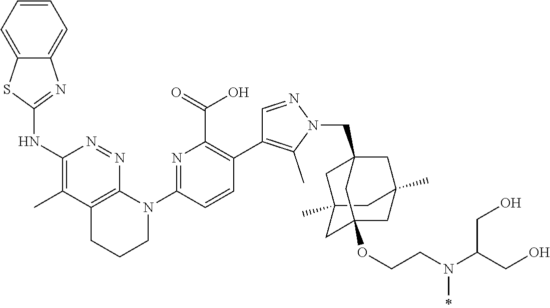 | D2-24a |
| 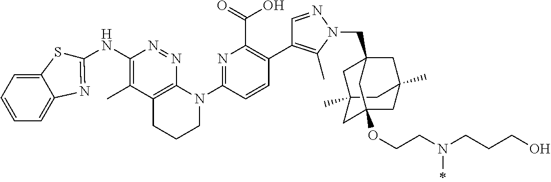 | D2-25a |
| 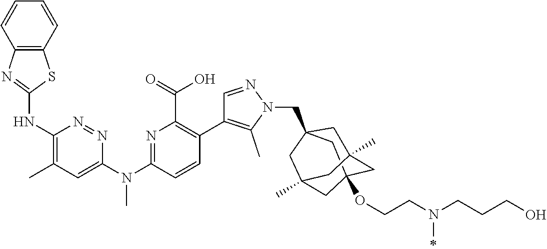 | D2-26a |
| 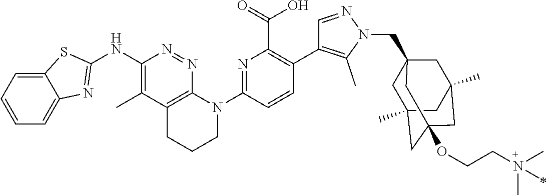 | D2-27a |
| 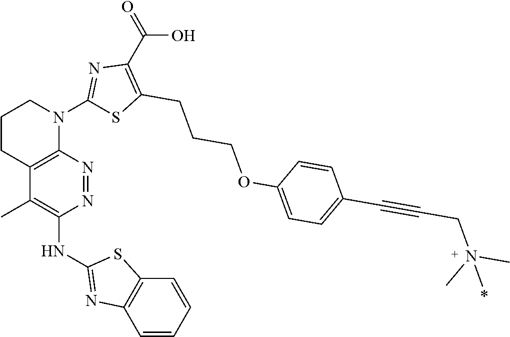 | D2-28a |
| 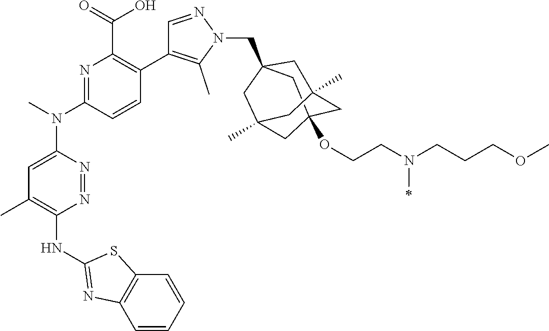 | D2-29a |
| 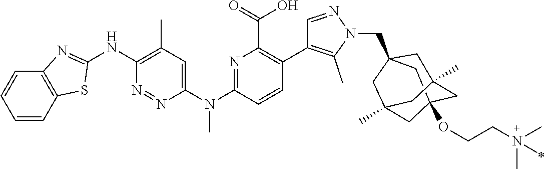 | D2-30a |
| 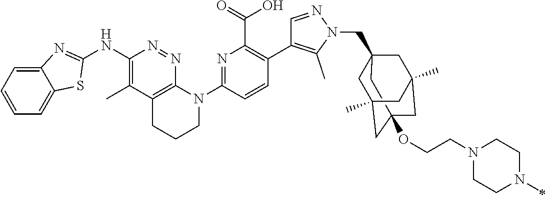 | D2-31a |
| 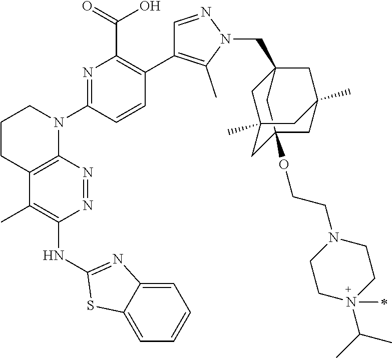 | D2-32a |
| 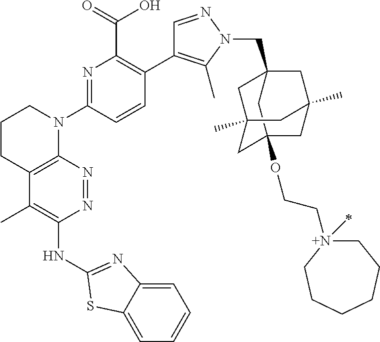 | D2-33a |
| 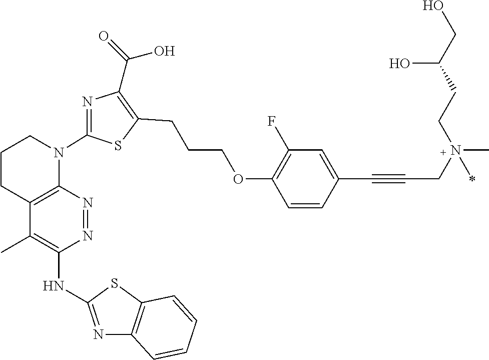 | D2-34a |
| 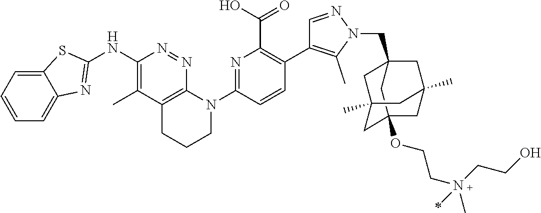 | D2-35a |
| 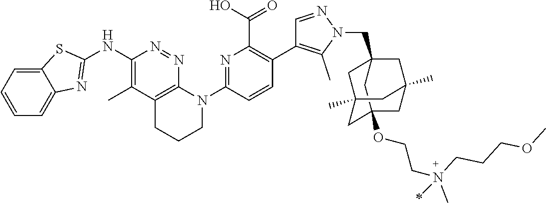 | D2-36a |
| 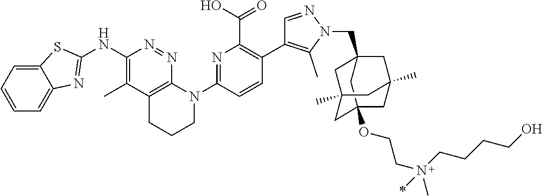 | D2-37a |
| 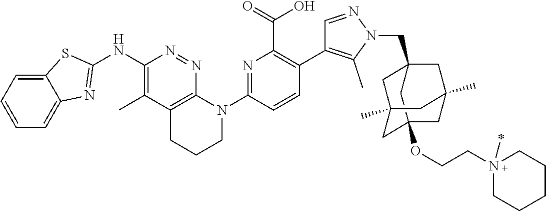 | D2-38a |
| 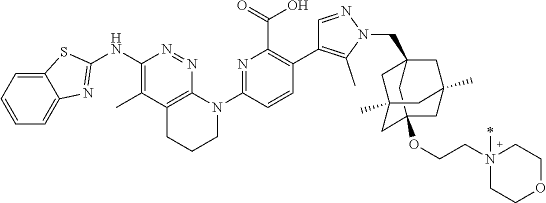 | D2-39a |
| 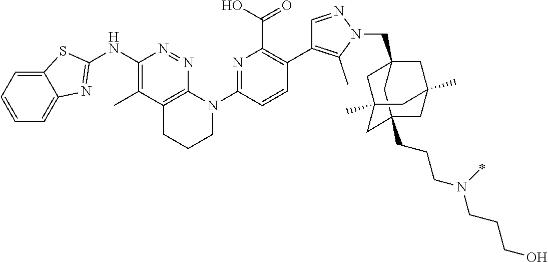 | D2-40a |
| 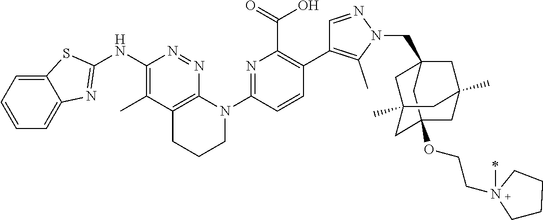 | D2-41a |
| 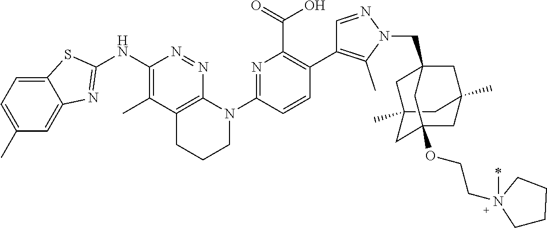 | D2-42a |
| 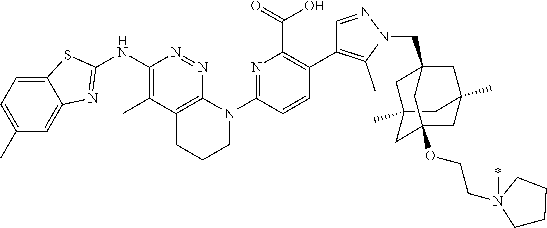 | D2-43a |
| 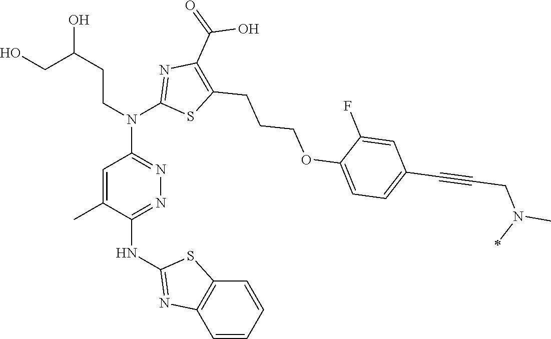 | D2-44a |
| 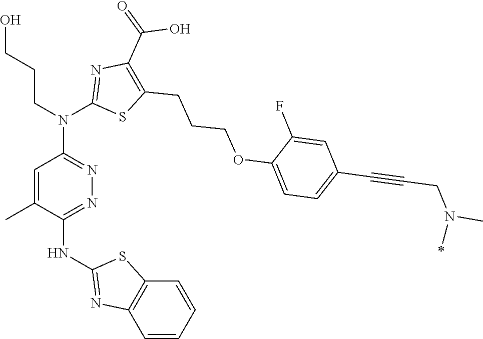 | D2-45a |
| 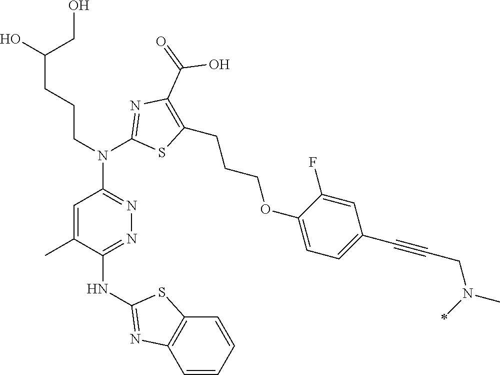 | D2-46a |
| 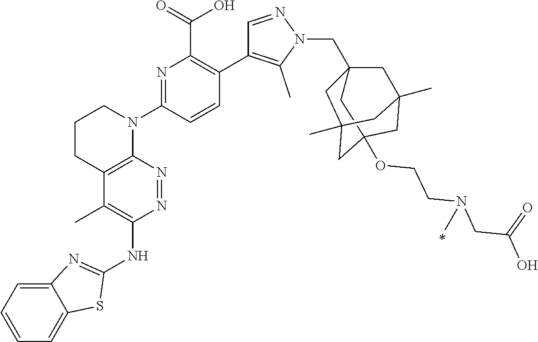 [SMILES: Cc1c(Nc2nc3ccccc3s2)nnc2c1CCCN2c1ccc(-c2cnn(CC34CC5(C)CC(C)(C3)CC(OCCN(C)CC(=O)O)(C5)C4)c2C)c(C(=O)O)n1] | D2-47a |
| 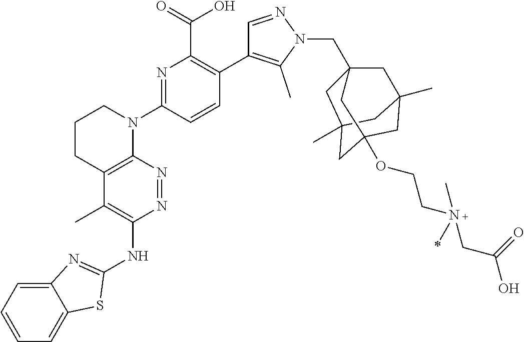 | D2-48a |
| 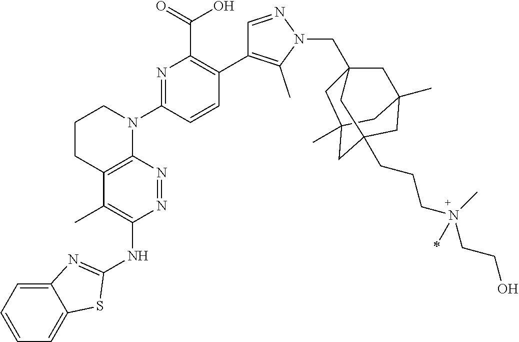 | D2-49a |
| 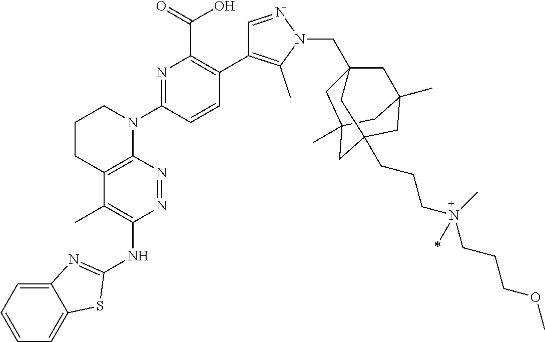 | D2-50a |
| 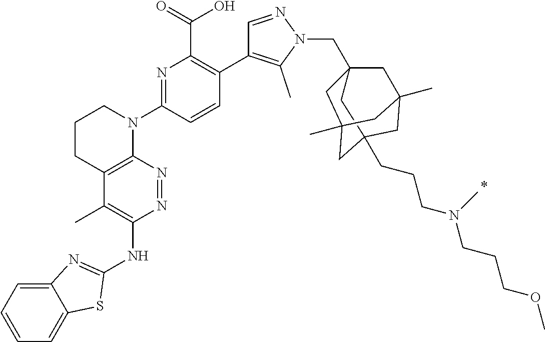 [SMILES: COCCCN(C)CCCC12CC3(C)CC(C)(C1)CC(Cn1ncc(-c4ccc(N5CCCc6c5nnc(Nc5nc7ccccc7s5)c6C)nc4C(=O)O)c1C)(C3)C2] | D2-51a |
| 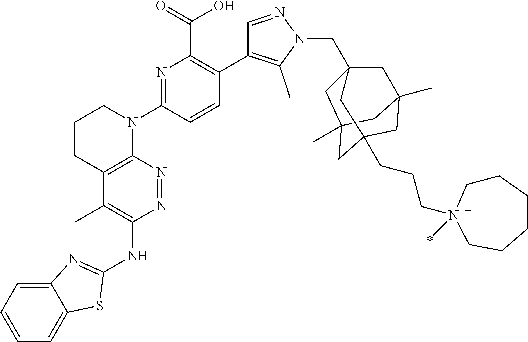 | D2-52a |
| 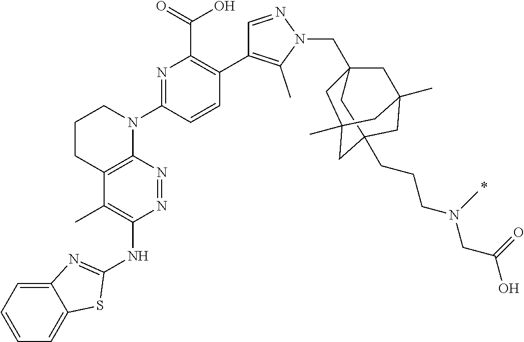 [SMILES: Cc1c(Nc2nc3ccccc3s2)nnc2c1CCCN2c1ccc(-c2cnn(CC34CC5(C)CC(C)(CC(CCCN(C)CC(=O)O)(C5)C3)C4)c2C)c(C(=O)O)n1] | D2-53a |
| 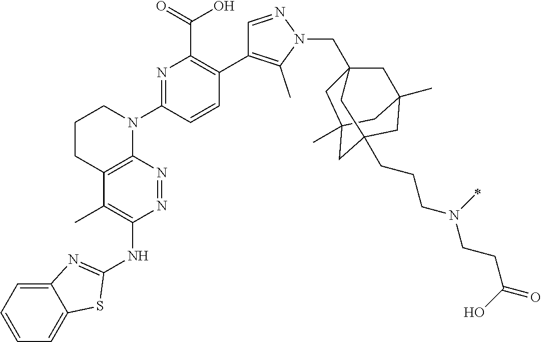 | D2-54a |
| 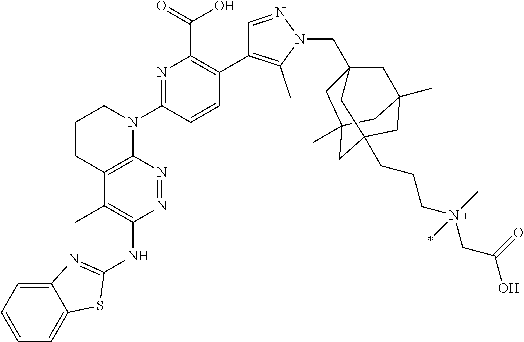 | D2-55a |
| 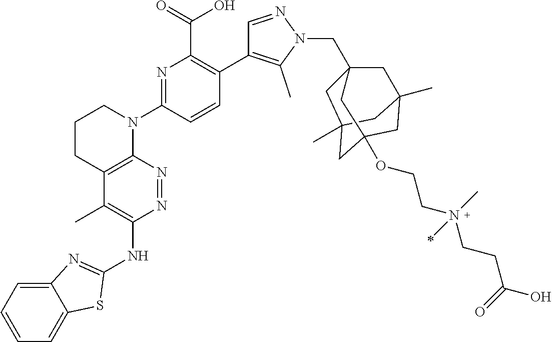 | D2-56a |
| 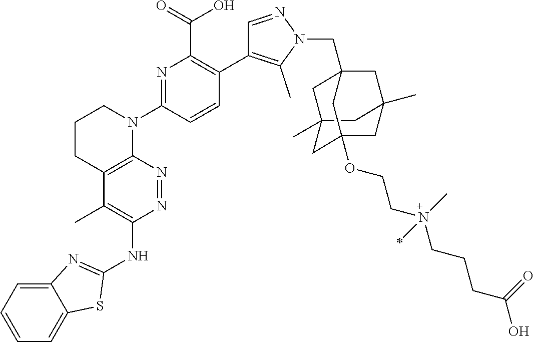 | D2-57a |
| 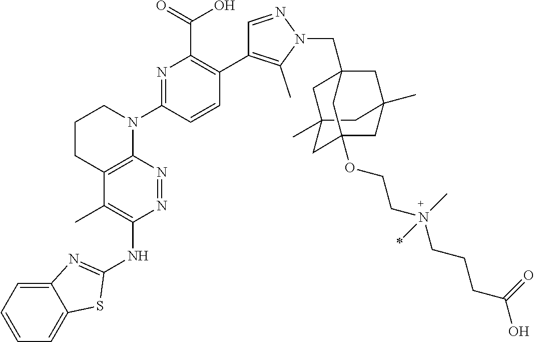 | D2-58a |
| 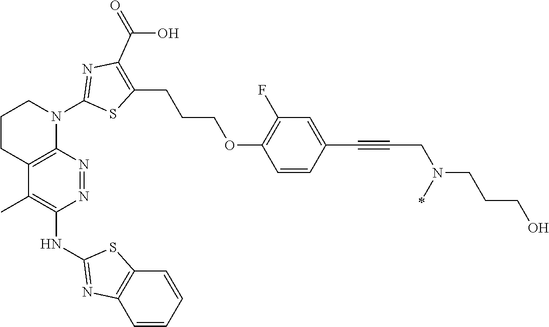 | D2-59a |
| 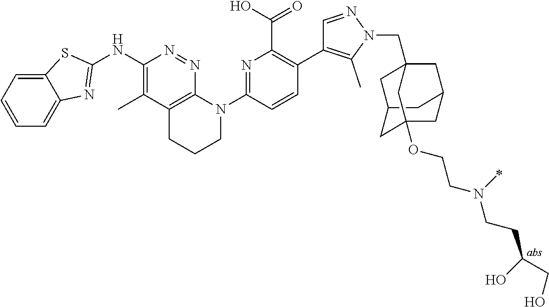 | D2-60a |
| 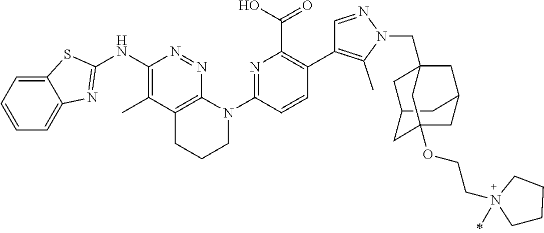 | D2-61a |
| 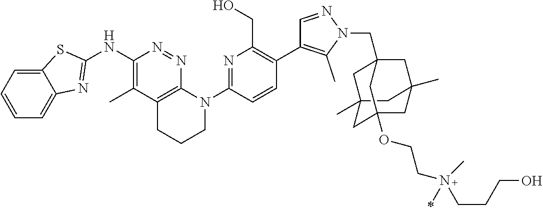 | D2-62a |
| 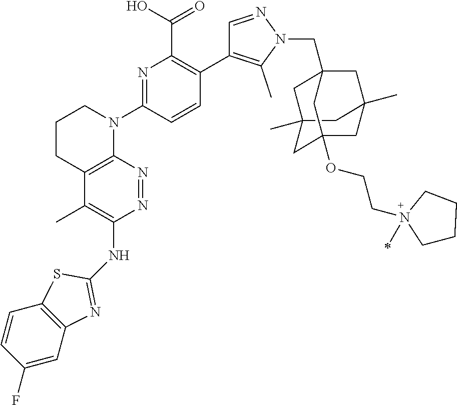 | D2-63a |
| 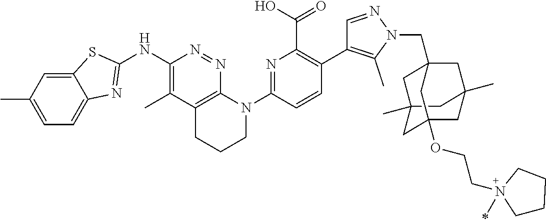 | D2-64a |
| 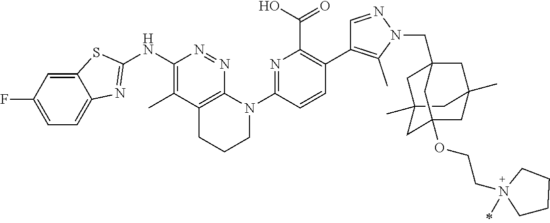 | D2-65a |
| 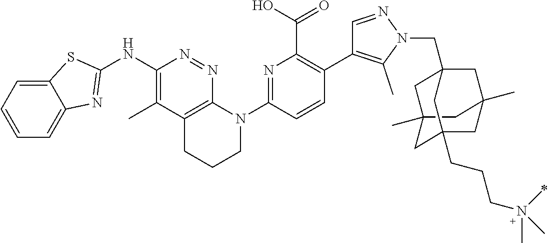 | D2-66a |
| 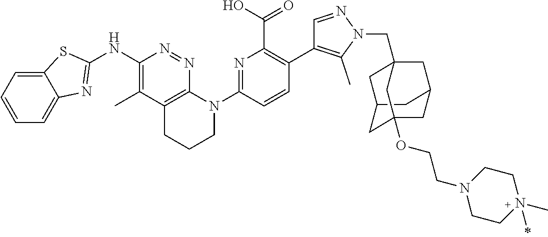 | D2-67a |
| 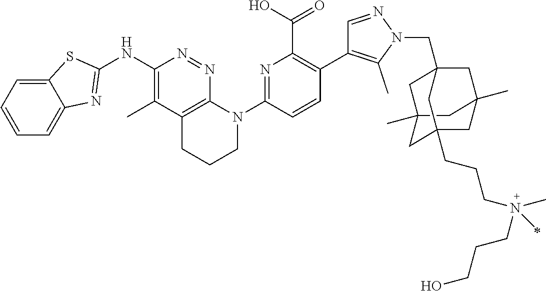 | D2-68a |
| 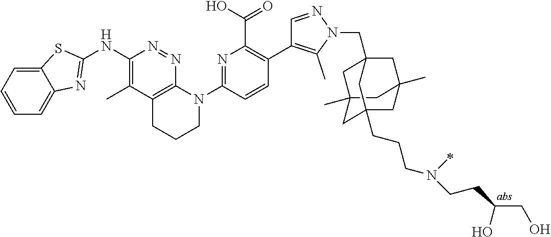 | D2-69a |
| 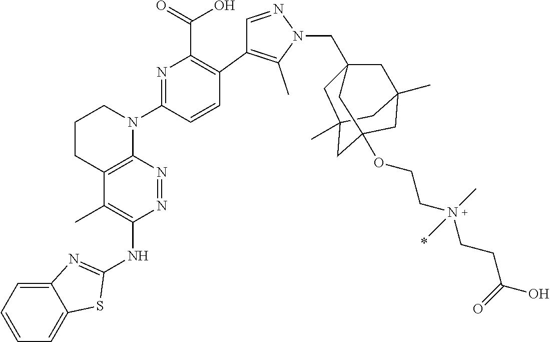 | D2-70a |
| 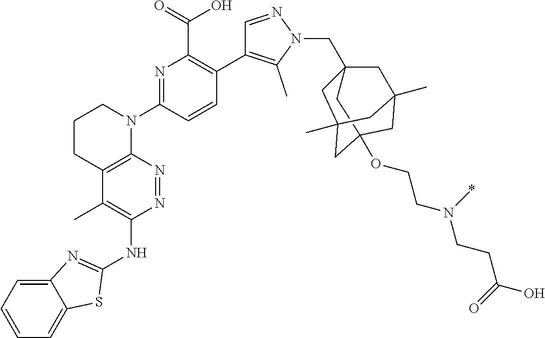 [SMILES: *N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CCC(=O)O] | D2-71a |
| 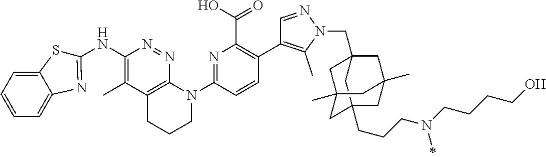 | D2-72a |
| 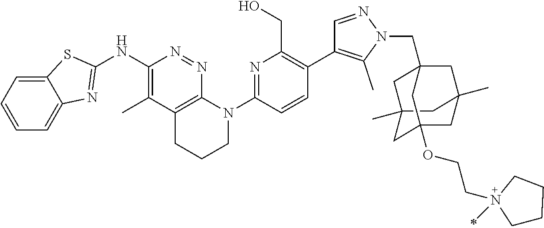 | D2-73a |
| 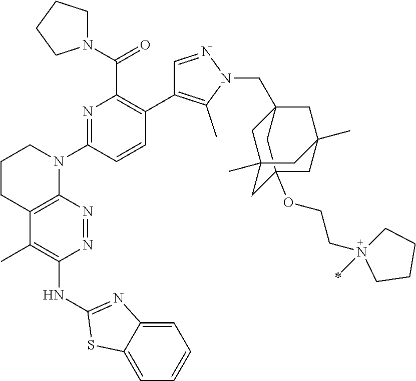 | D2-74a |
| 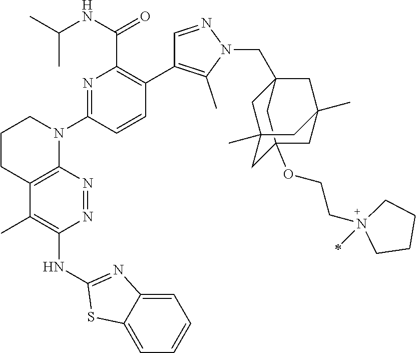 | D2-75a |
| 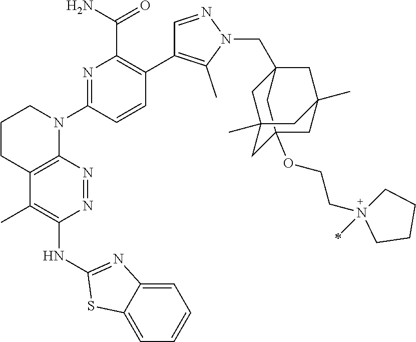 | D2-76a |

wherein [symbol] represents a bond to the linker.

In some embodiments, D¹ and/or D² in the ADC compounds disclosed herein (e.g., ADCs of Formula (A), (B), (C), (D1), (D2), or (D3) in the present disclosure) independently comprises a formula selected from any one of the formulae in Table A3a, or an enantiomer, a diastereoisomer, and/or a pharmaceutically acceptable salt of any one of the foregoing.

TABLE A3a

| | |
|---|---|
| 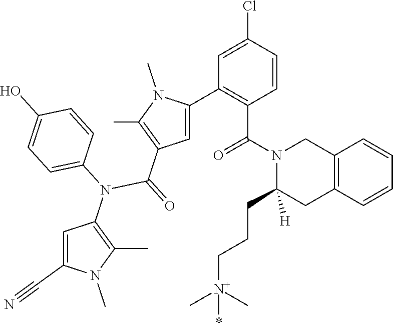 [SMILES: *[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C] | D3-1a |
| 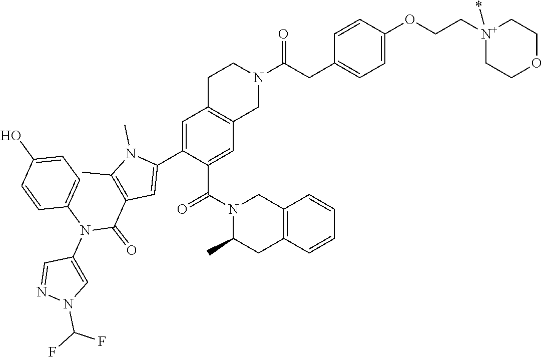 [SMILES: *[N+]1(CCOc2ccc(CC(=O)N3CCc4cc(-c5cc(C(=O)N(c6ccc(O)cc6)c6cnn(C(F)F)c6)c(C)n5C)c(C(=O)N5Cc6ccccc6C[C@H]5C)cc4C3)cc2)CCOCC1] | D3-2a |
| 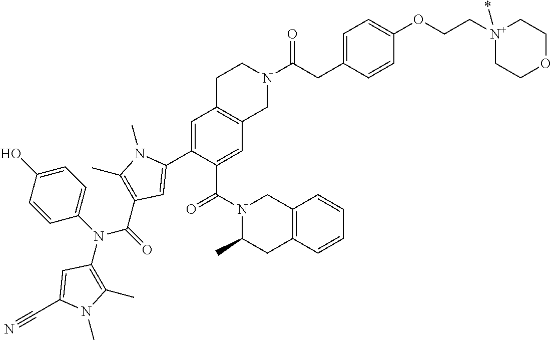 [SMILES: *[N+]1(CCOc2ccc(CC(=O)N3CCc4cc(-c5cc(C(=O)N(c6ccc(O)cc6)c6cc(C#N)n(C)c6C)c(C)n5C)c(C(=O)N5Cc6ccccc6C[C@H]5C)cc4C3)cc2)CCOCC1] | D3-3a |
| 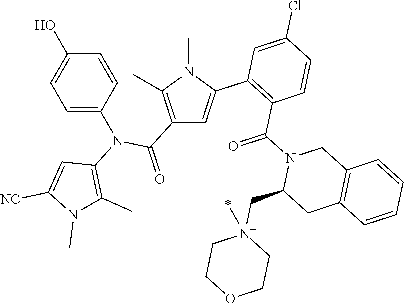 [SMILES: *[N+]1(C[C@@H]2Cc3ccccc3CN2C(=O)c2ccc(Cl)cc2-c2cc(C(=O)N(c3ccc(O)cc3)c3cc(C#N)n(C)c3C)c(C)n2C)CCOCC1] | D3-4a |
| 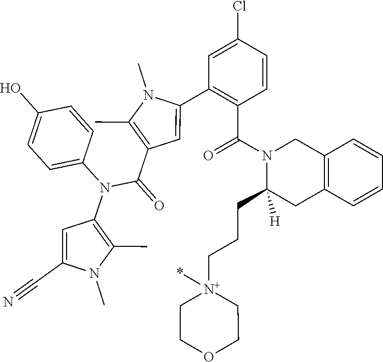 [SMILES: *[N+]1(CCC[C@@H]2Cc3ccccc3CN2C(=O)c2ccc(Cl)cc2-c2cc(C(=O)N(c3ccc(O)cc3)c3cc(C#N)n(C)c3C)c(C)n2C)CCOCC1] | D3-5a |
| 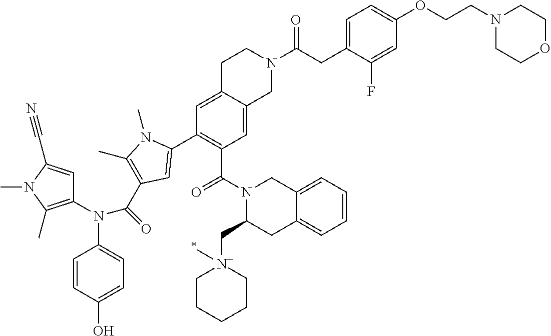 [SMILES: Cc1c(N(C(=O)c2cc(-c3cc4c(cc3C(=O)N3Cc5ccccc5C[C@H]3C[N+]3(C)CCCCC3)CN(C(=O)Cc3ccc(OCCN5CCOCC5)cc3F)CC4)n(C)c2C)c2ccc(O)cc2)cc(C#N)n1C] | D3-6a |
| 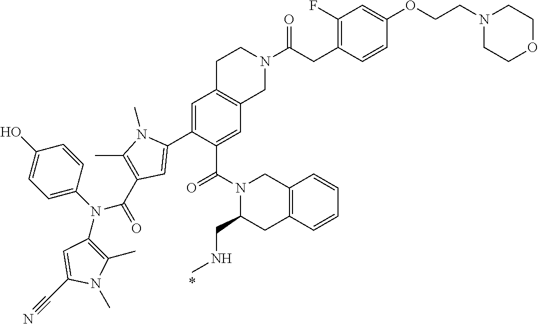 [SMILES: CNC[C@@H]1Cc2ccccc2CN1C(=O)c1cc2c(cc1-c1cc(C(=O)N(c3ccc(O)cc3)c3cc(C#N)n(C)c3C)c(C)n1C)CCN(C(=O)Cc1ccc(OCCN3CCOCC3)cc1F)C2] | D3-7a |
| 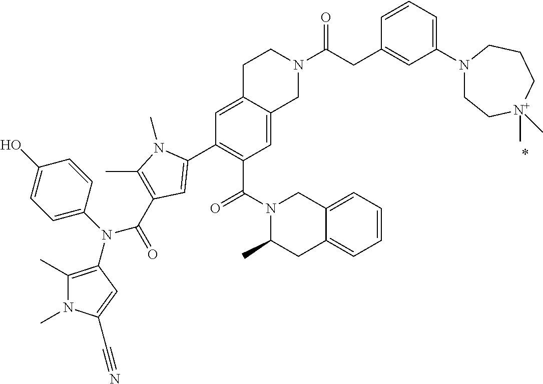 [SMILES: Cc1c(N(C(=O)c2cc(-c3cc4c(cc3C(=O)N3Cc5ccccc5C[C@H]3C)CN(C(=O)Cc3cccc(N5CCC[N+](C)(C)CC5)c3)CC4)n(C)c2C)c2ccc(O)cc2)cc(C#N)n1C] | D3-8a |
| 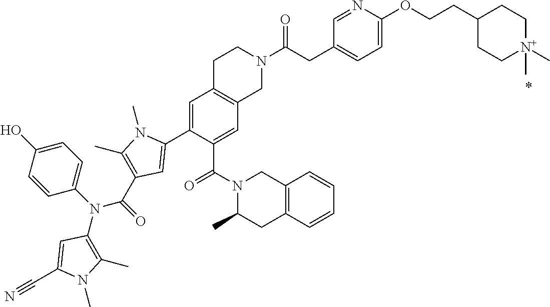 [SMILES: Cc1c(N(C(=O)c2cc(-c3cc4c(cc3C(=O)N3Cc5ccccc5C[C@H]3C)CN(C(=O)Cc3ccc(OCCC5CC[N+](C)(C)CC5)nc3)CC4)n(C)c2C)c2ccc(O)cc2)cc(C#N)n1C] | D3-9a |
| 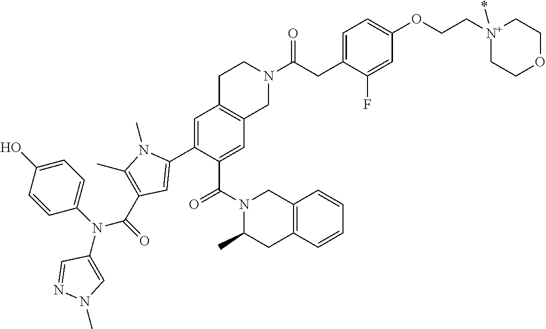 [SMILES: *[N+]1(CCOc2ccc(CC(=O)N3CCc4cc(-c5cc(C(=O)N(c6ccc(O)cc6)c6cnn(C)c6)c(C)n5C)c(C(=O)N5Cc6ccccc6C[C@H]5C)cc4C3)c(F)c2)CCOCC1] | D3-10a |
| 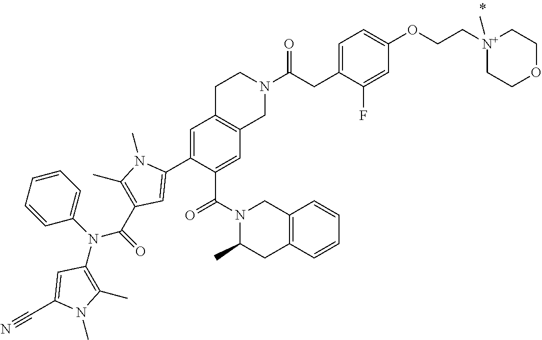 [SMILES: Cc1c(N(C(=O)c2cc(-c3cc4c(cc3C(=O)N3Cc5ccccc5C[C@H]3C)CN(C(=O)Cc3ccc(OCC[N+]5(C)CCOCC5)cc3F)CC4)n(C)c2C)c2ccccc2)cc(C#N)n1C] | D3-11a |
| 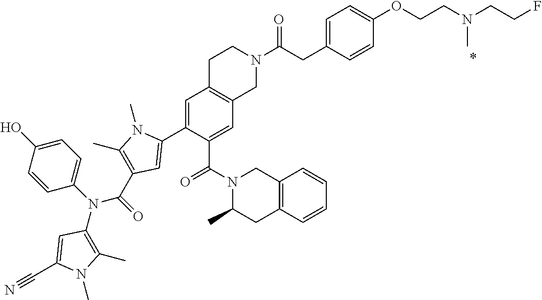 [SMILES: Cc1c(N(C(=O)c2cc(-c3cc4c(cc3C(=O)N3Cc5ccccc5C[C@H]3C)CN(C(=O)Cc3ccc(OCCN(C)CCF)cc3)CC4)n(C)c2C)c2ccc(O)cc2)cc(C#N)n1C] | D3-12a |
| 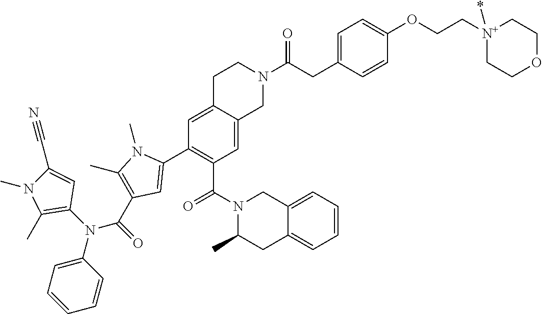 [SMILES: *[N+]1(CCOc2ccc(CC(=O)N3CCc4cc(-c5cc(C(=O)N(c6ccccc6)c6cc(C#N)n(C)c6C)c(C)n5C)c(C(=O)N5Cc6ccccc6C[C@H]5C)cc4C3)cc2)CCOCC1] | D3-13a |
| 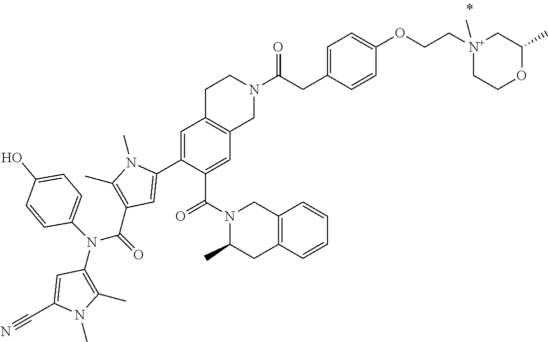 [SMILES: Cc1c(N(C(=O)c2cc(-c3cc4c(cc3C(=O)N3Cc5ccccc5C[C@H]3C)CN(C(=O)Cc3ccc(OCC[N+]5(C)CCO[C@@H](C)C5)cc3)CC4)n(C)c2C)c2ccc(O)cc2)cc(C#N)n1C] | D3-14a |
| 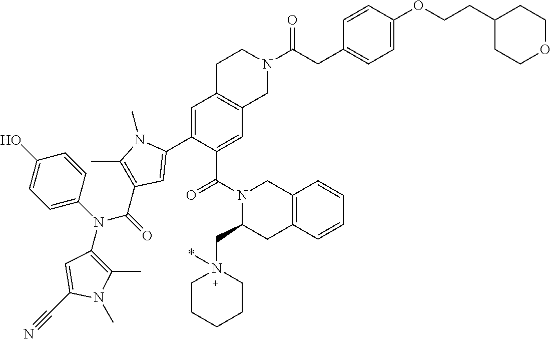 | D3-15a |
| 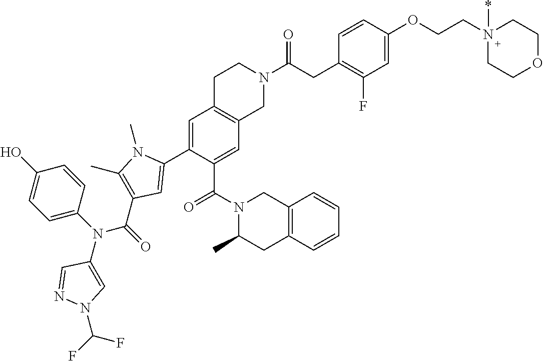 | D3-16a |
| 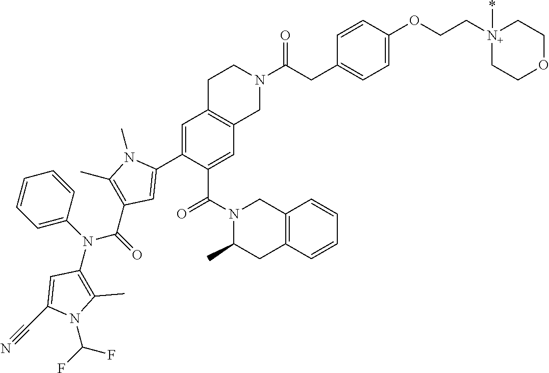 [SMILES: *[N+]1(CCOc2ccc(CC(=O)N3CCc4cc(-c5cc(C(=O)N(c6ccccc6)c6cc(C#N)n(C(F)F)c6C)c(C)n5C)c(C(=O)N5Cc6ccccc6C[C@H]5C)cc4C3)cc2)CCOCC1] | D3-17a |

wherein [symbol] represents a bond to the linker.

[133]In some embodiments, D¹ and/or D² in the ADC compounds disclosed herein (e.g., ADCs of Formula (A), (B), (C), (D1), (D2), or (D3) in the present disclosure) independently comprises a formula selected from any one of the formulae in TableA4a, or an enantiomer, a diastereoisomer, and/or a pharmaceutically acceptable salt of any one of the foregoing.

TABLE A4a

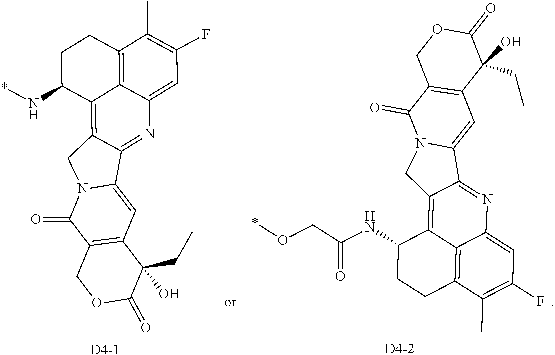

wherein [symbol] represents a bond to the linker.

In some embodiments,

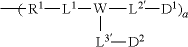

is formed from a compound selected from Table B or an enantiomer, a diastereoisomer, and/or a pharmaceutically acceptable salt thereof.

In some embodiments, the maleimide group

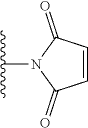 [SMILES: *N1C(=O)C=CC1=O]

in the compound of Table B form a covalent bond with the antibody or antigen-binding fragment thereof (Ab) to form the ADC compound of formula (A) comprising

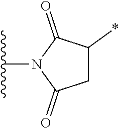 [SMILES: *C1CC(=O)N(*)C1=O]

moiety, wherein * indicates the connection point to Ab. For compounds in Table A1, Table A2, Table A3, Table B, and Table C, depending on their electronic charge, these compounds can contain one pharmaceutically acceptable monovalent anionic counterion Mi. In some embodiments, the monovalent anionic counterion Mⱼ can be selected from bromide, chloride, iodide, acetate, trifluoroacetate, benzoate, mesylate, tosylate, triflate, formate, or the like. In some embodiments, the monovalent anionic counterion Mⱼ is trifluoroacetate or formate.

TABLE B
Exemplary Linker Drug Groups

| Name | Structure of Linker-Drug |
|---|---|
| P1-L1- P1 | 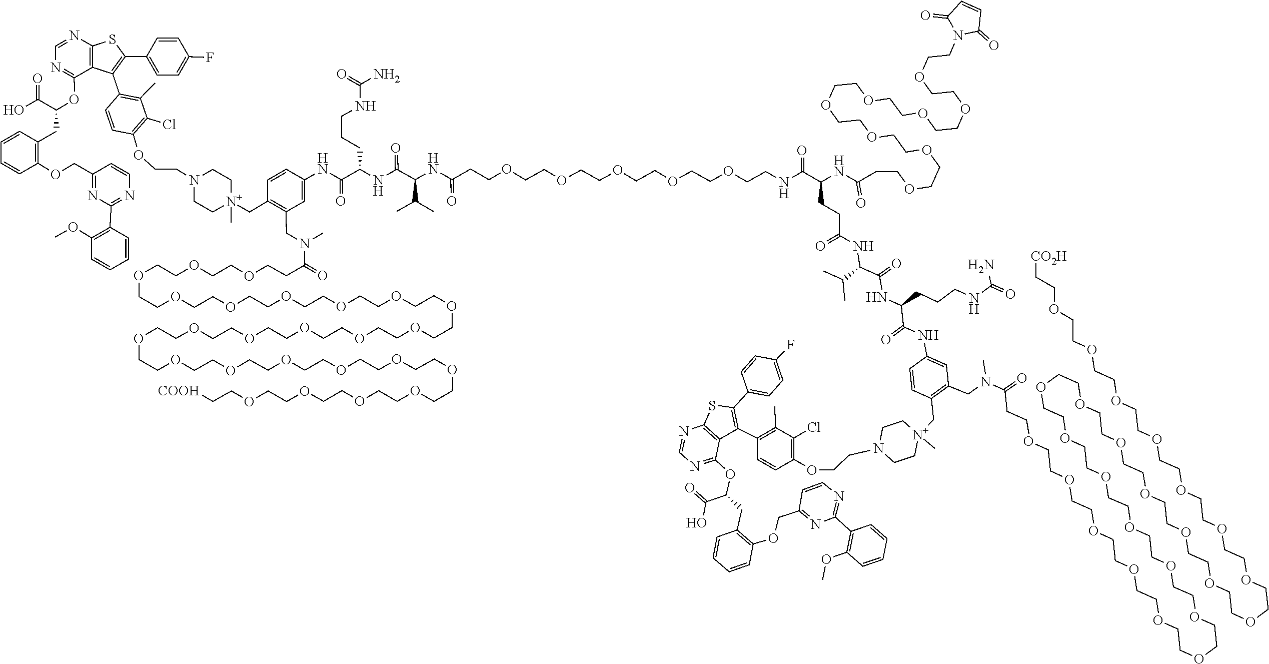 |
| P1-L2- P1 | 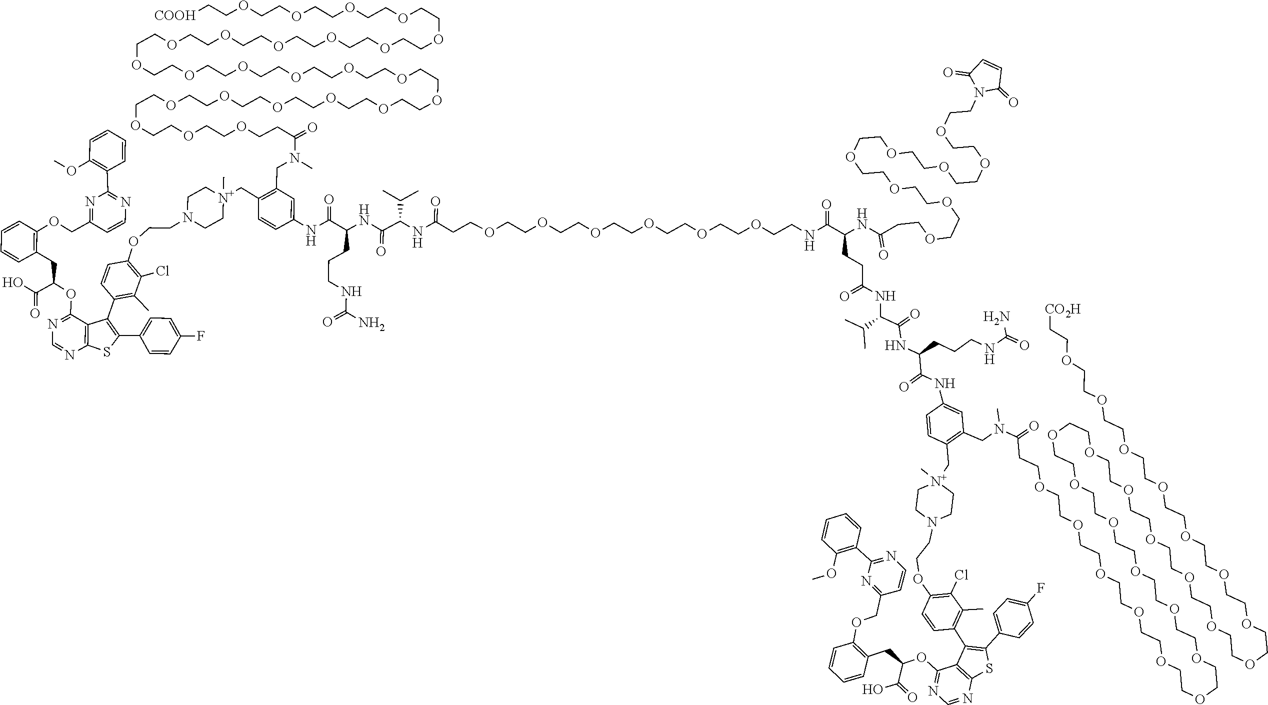 |
| P1-L3- P1 | 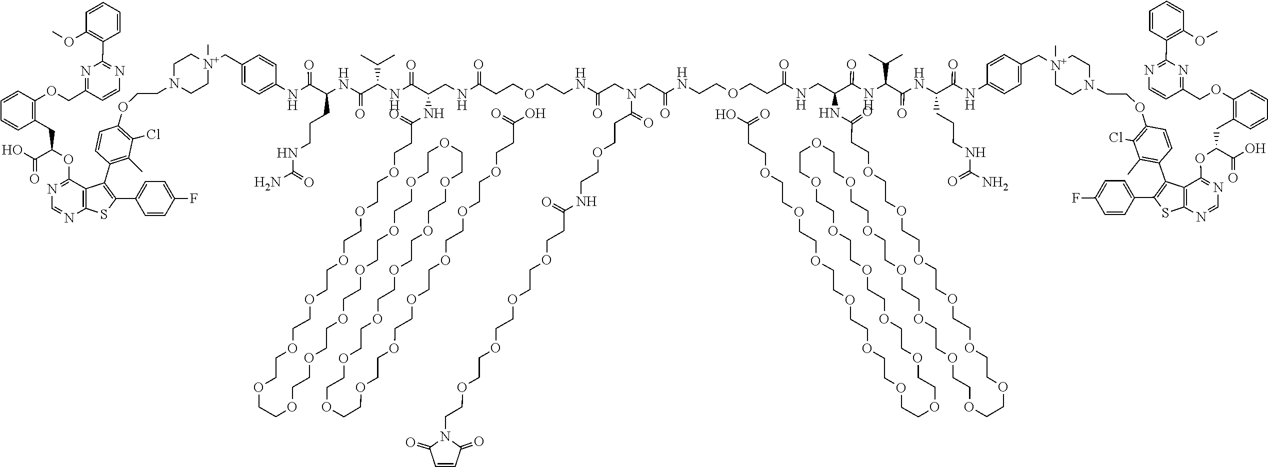 |
| P1-L4- P1 | 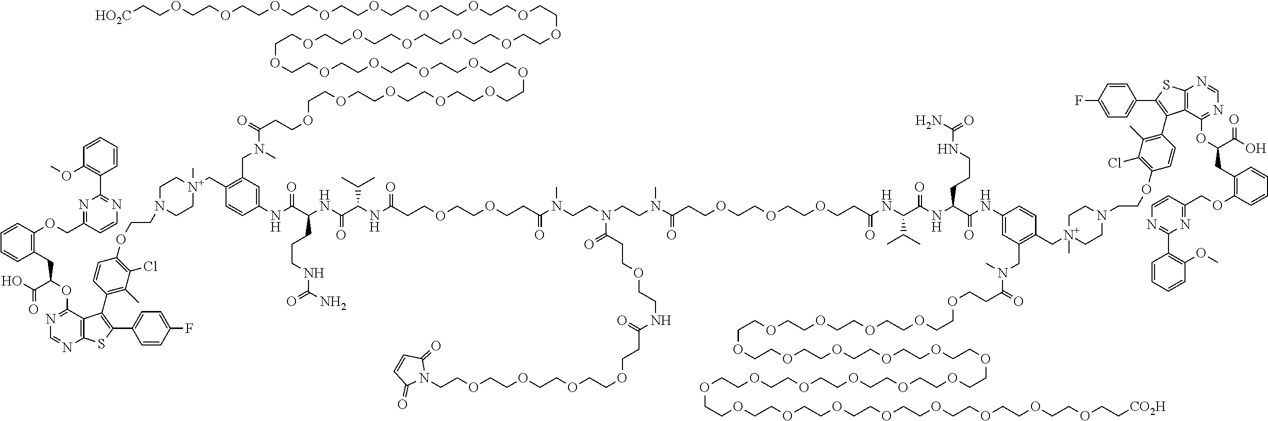 |
| P1-L5- P1 | 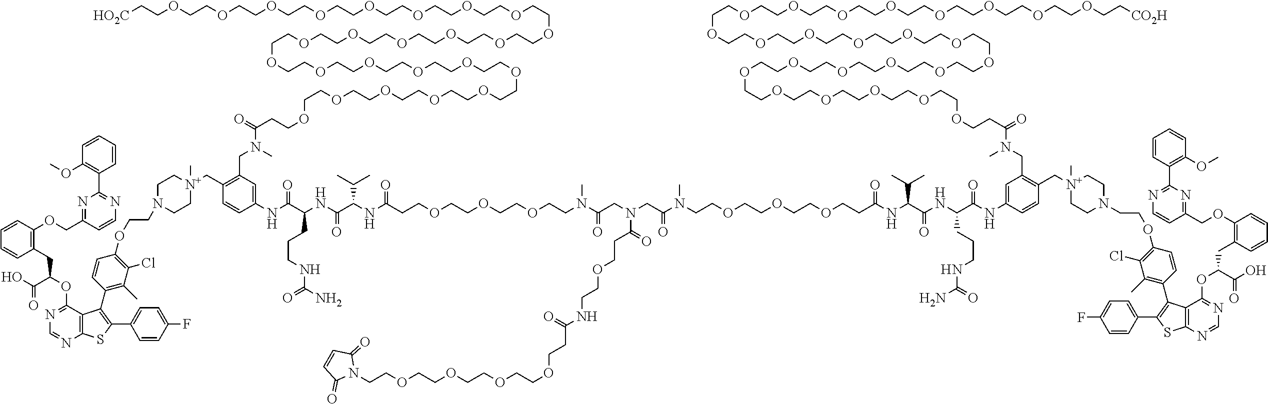 |
| P1-L6- P1 | 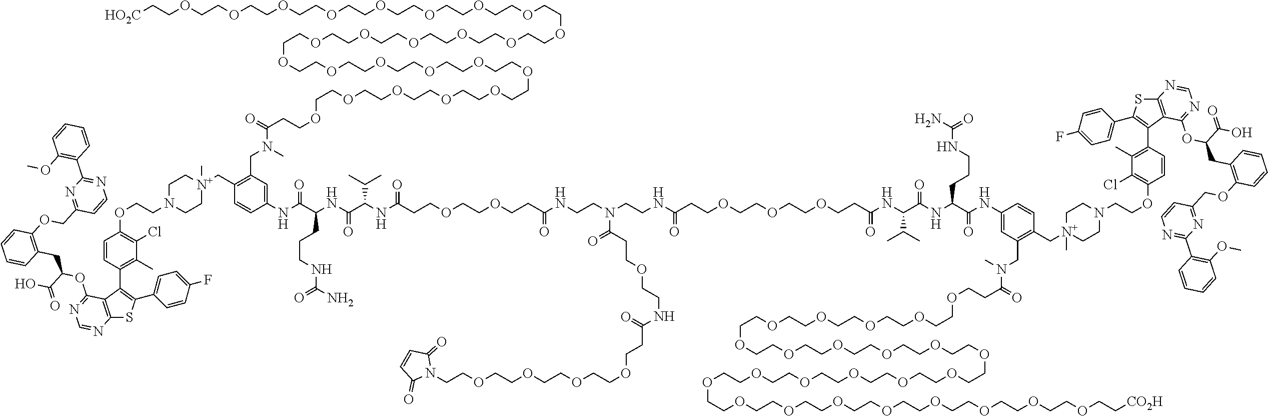 |
| P1-L7- P1 | 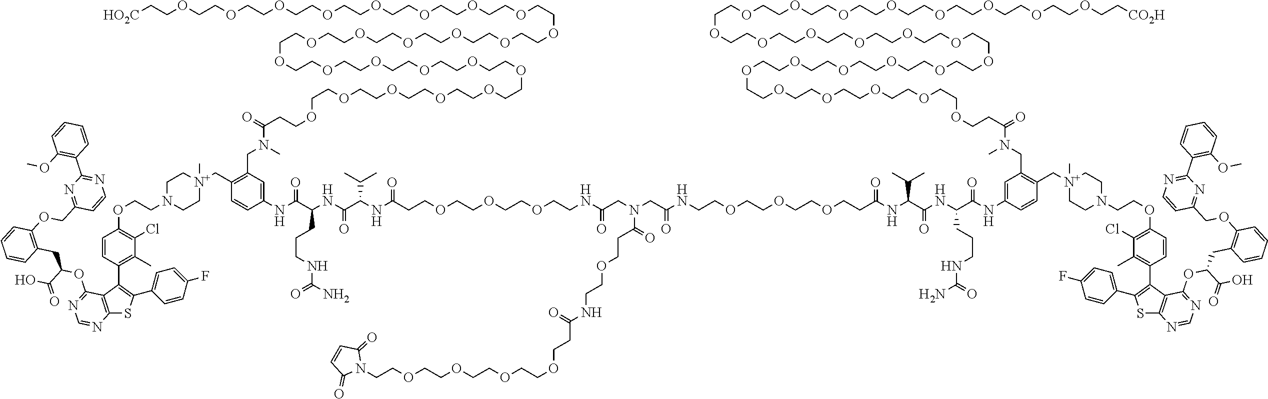 |
| P1-L8- P1 | 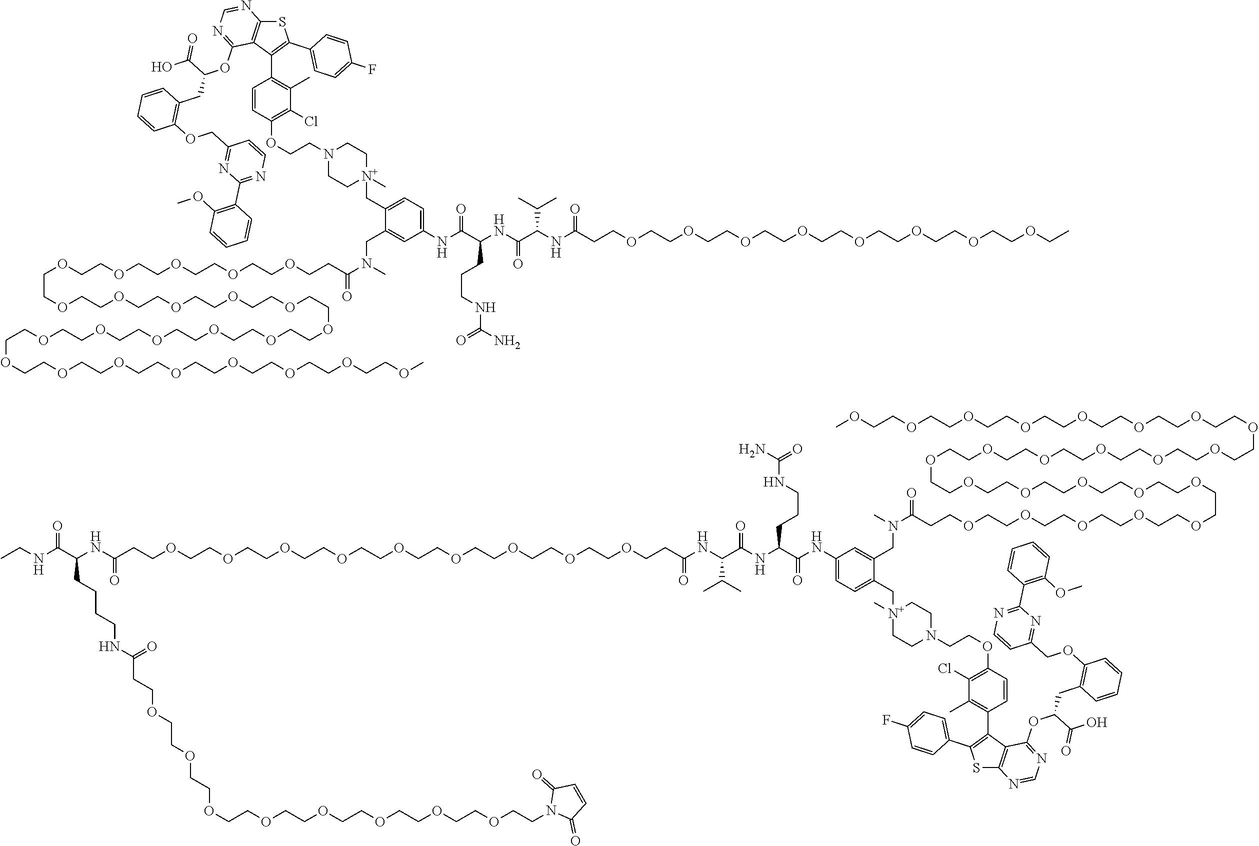 |
| P1-L9- P1 | 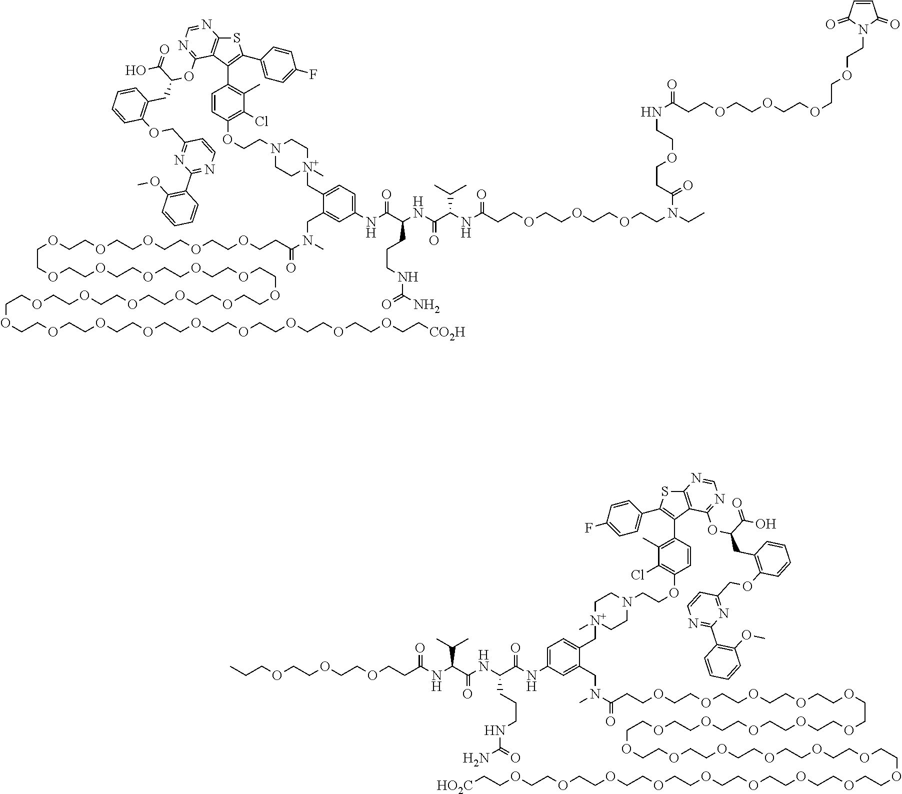 |
| P1-L10- P1 | 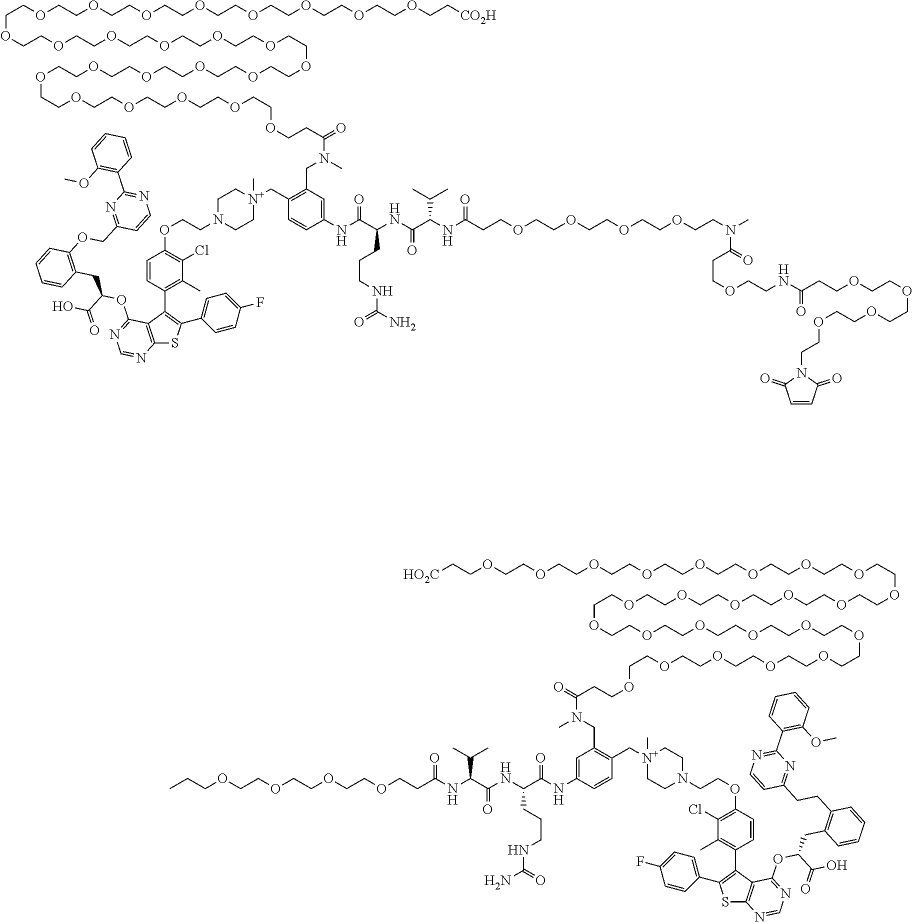 |
| P1-L11- P1 | 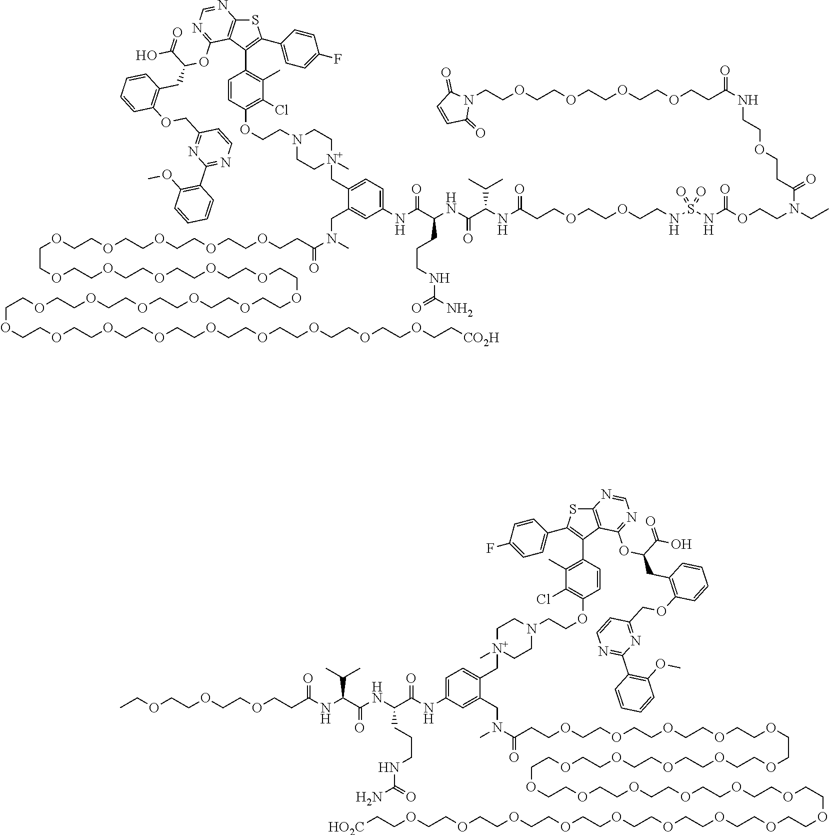 |
| P1-L12- P2 | 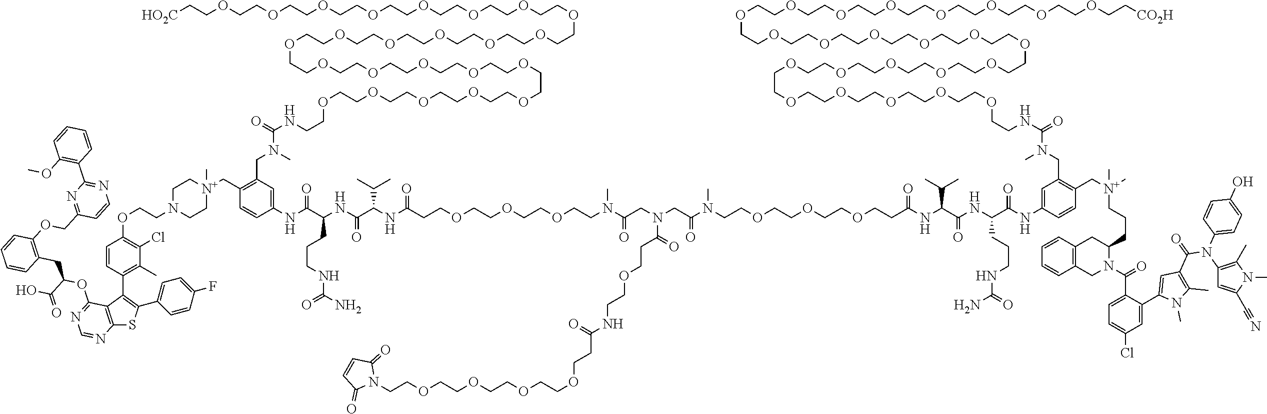 |
| P1-L12- P3 | 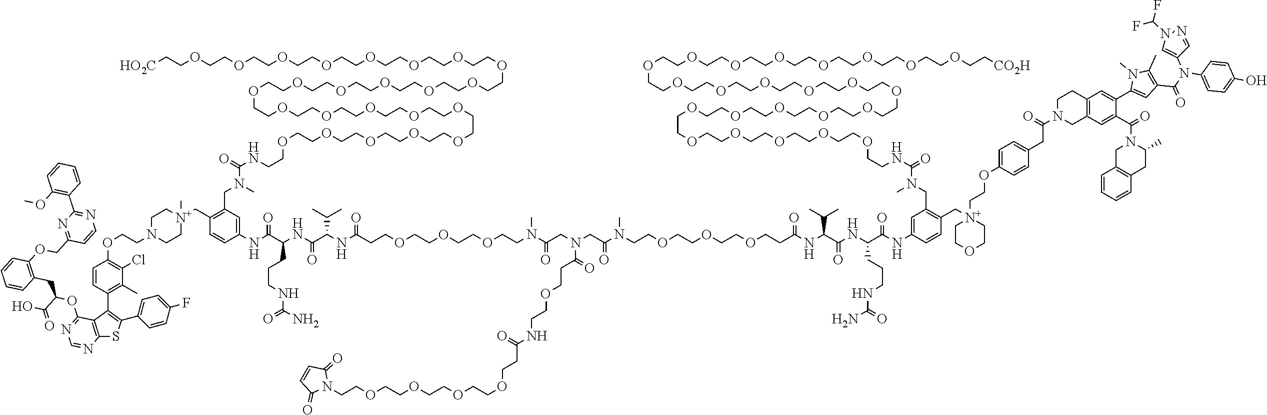 |
| P1-L12- P4 | 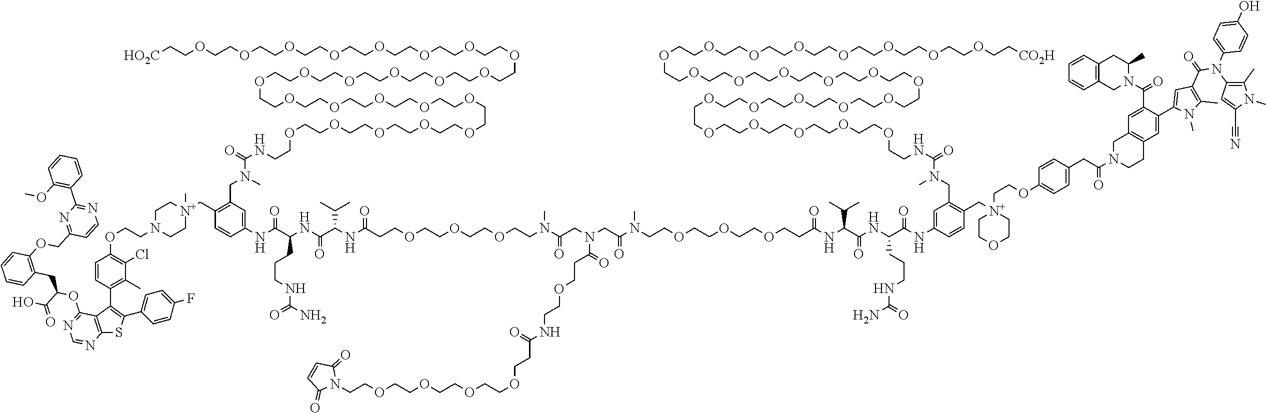 |
| P5-L12- P5 | 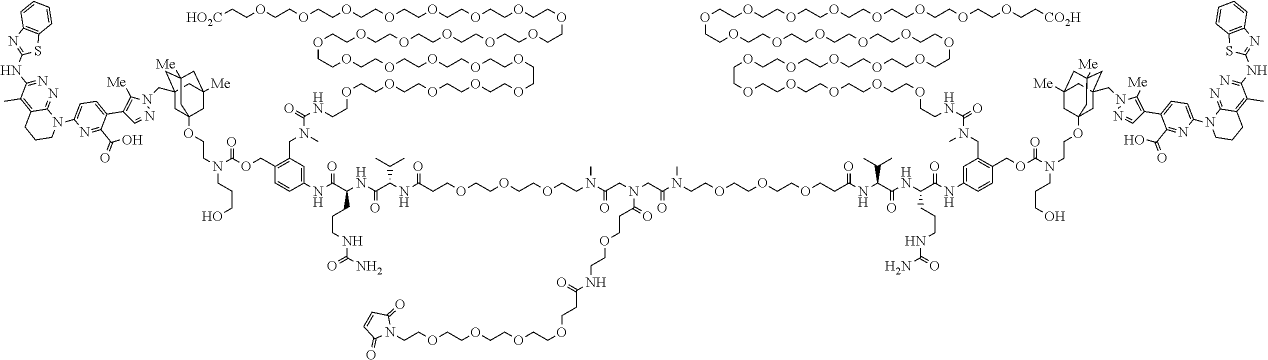 |
| P5-L12- P7 | 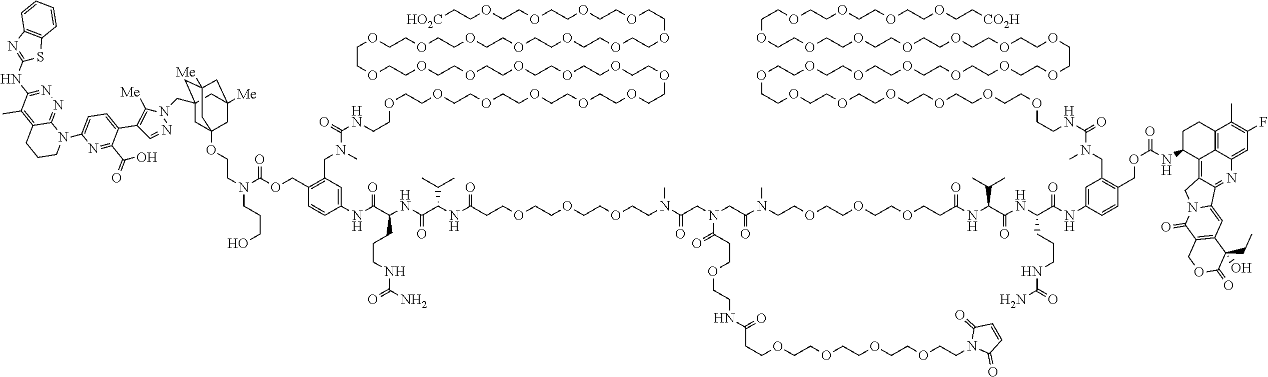 |
| P5-L12- P4 | 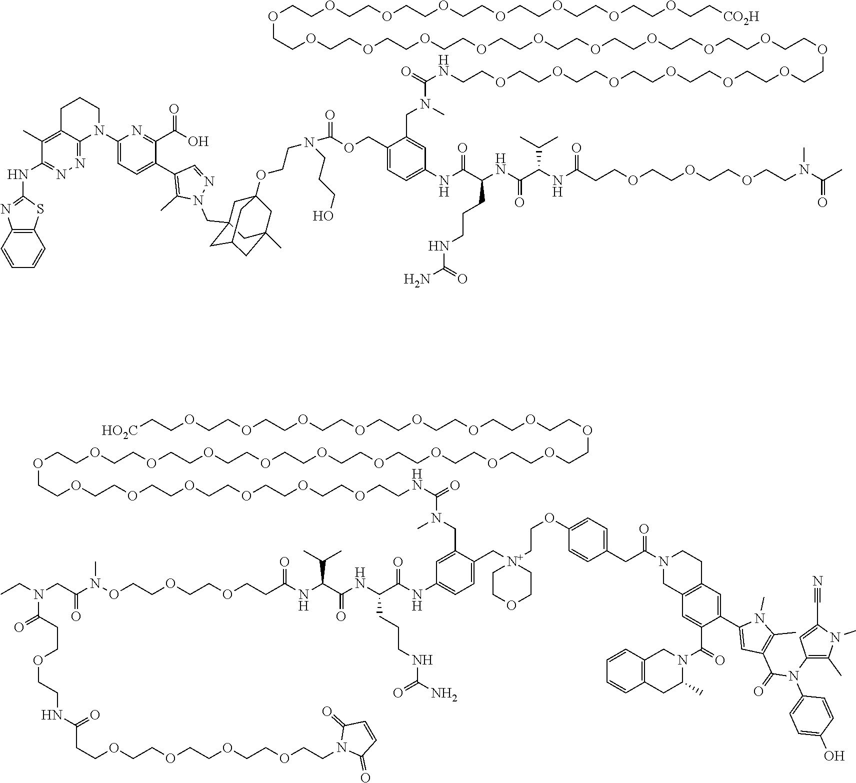 |
| P5-L12- P8 | 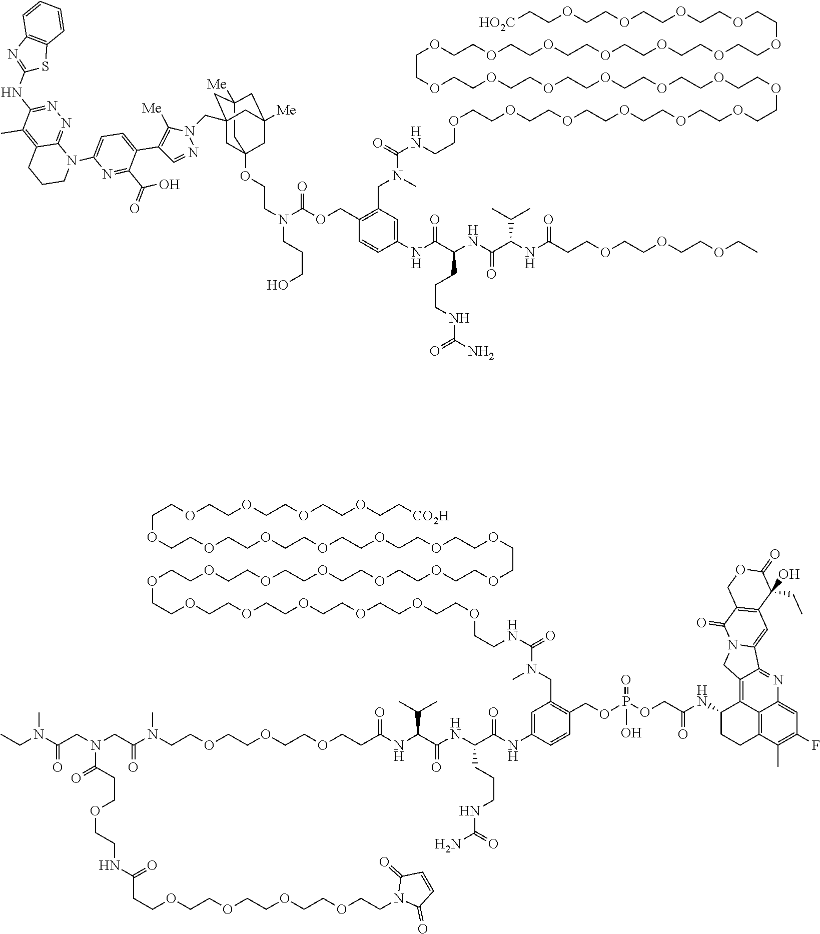 |
| P5-L37- P8 | 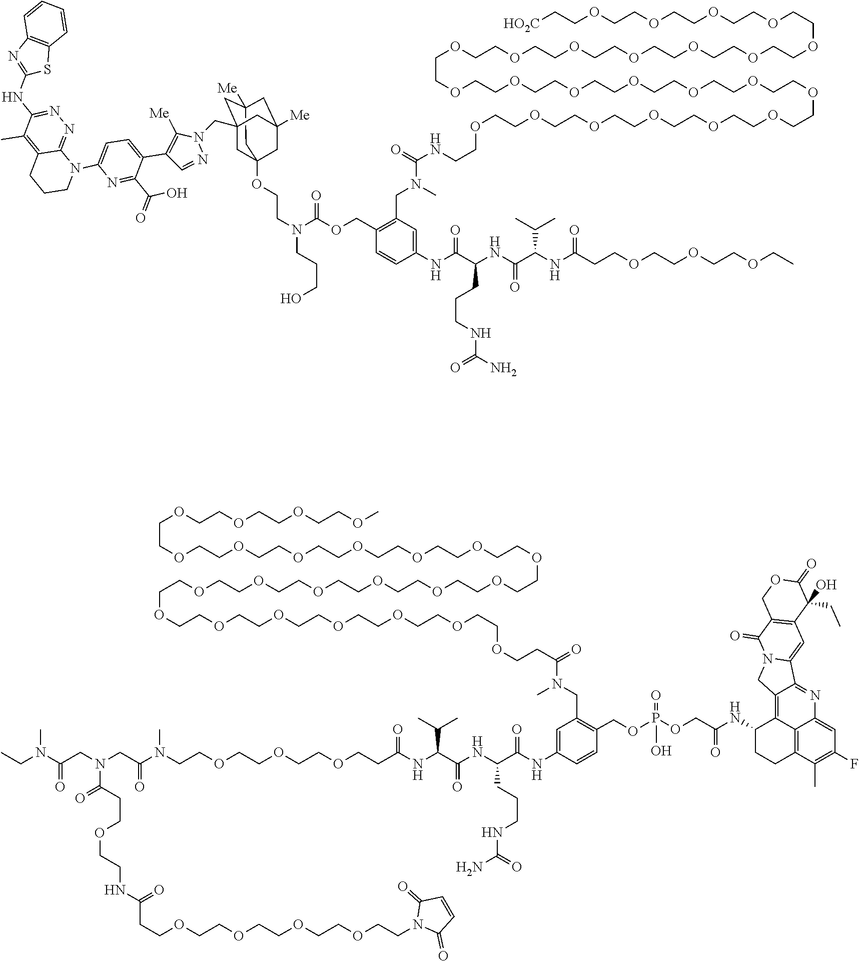 |
| P2-L13- P1 | 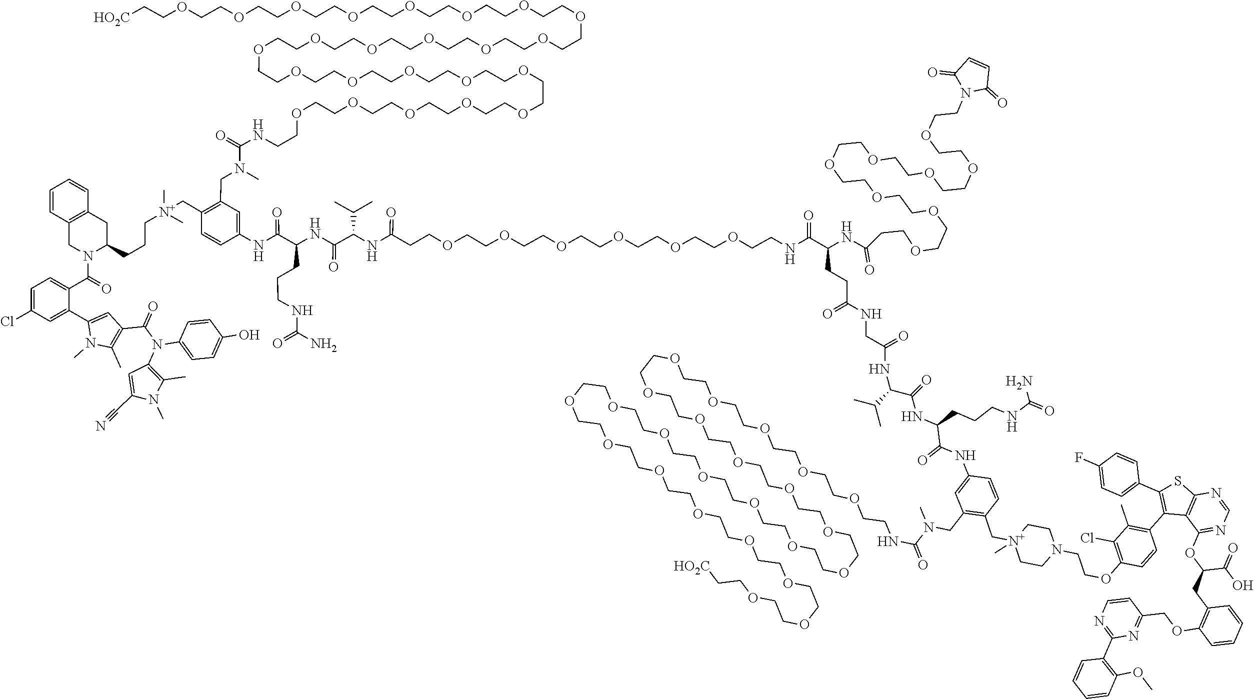 |
| P2-L14- P1 | 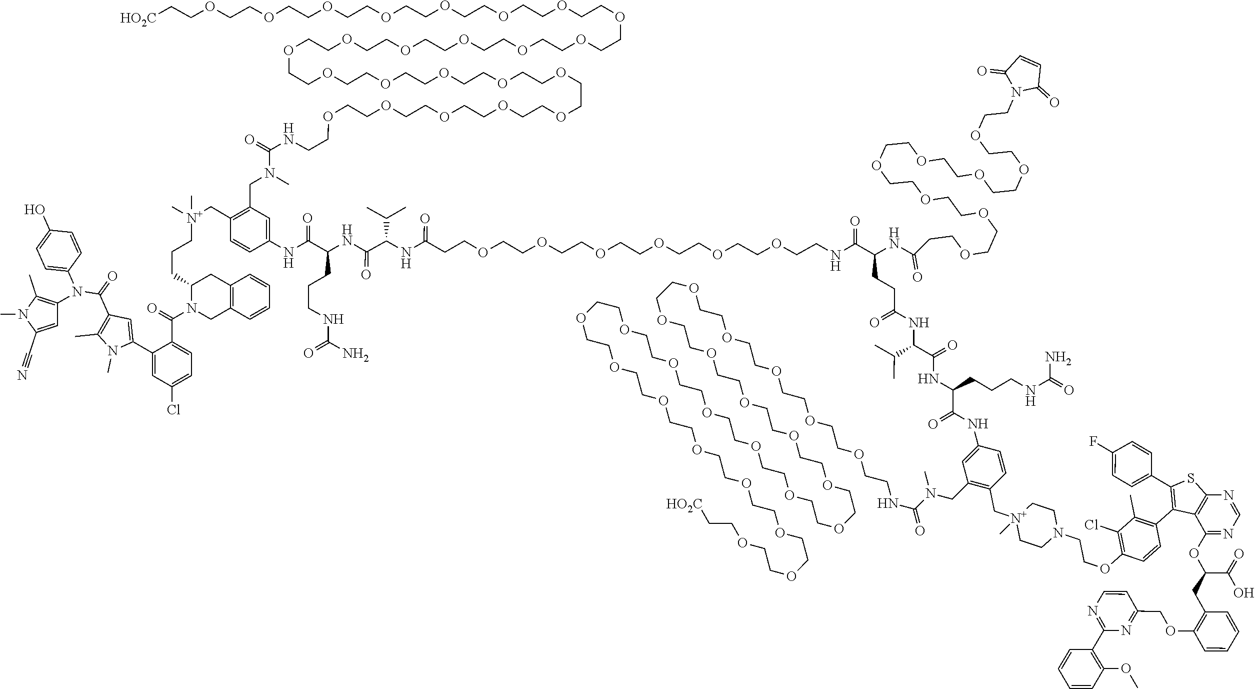 |
| P1-L15- P2 | 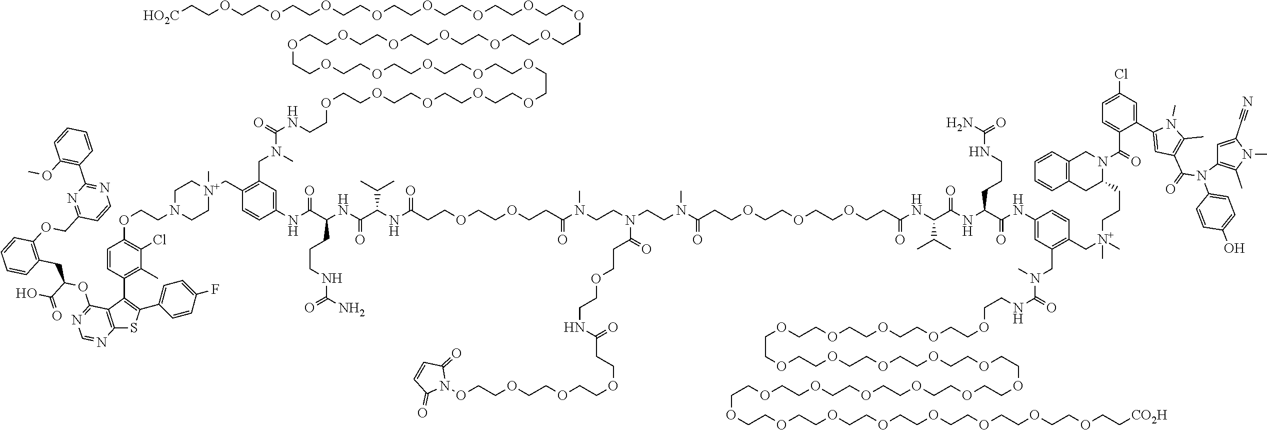 |
| P1-L16- P2 | 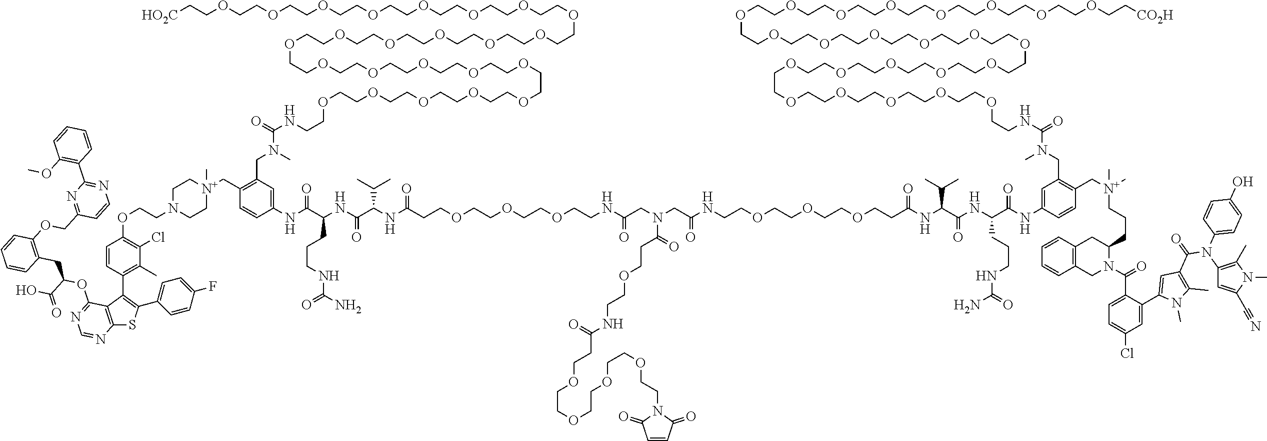 |
| P1-L17- P2 | 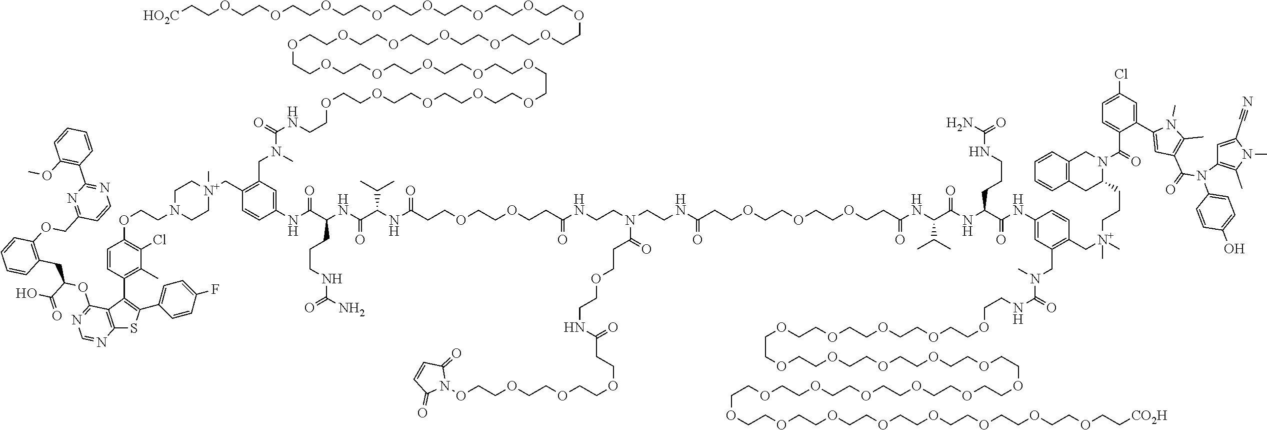 |
| P1-L17- P3 | 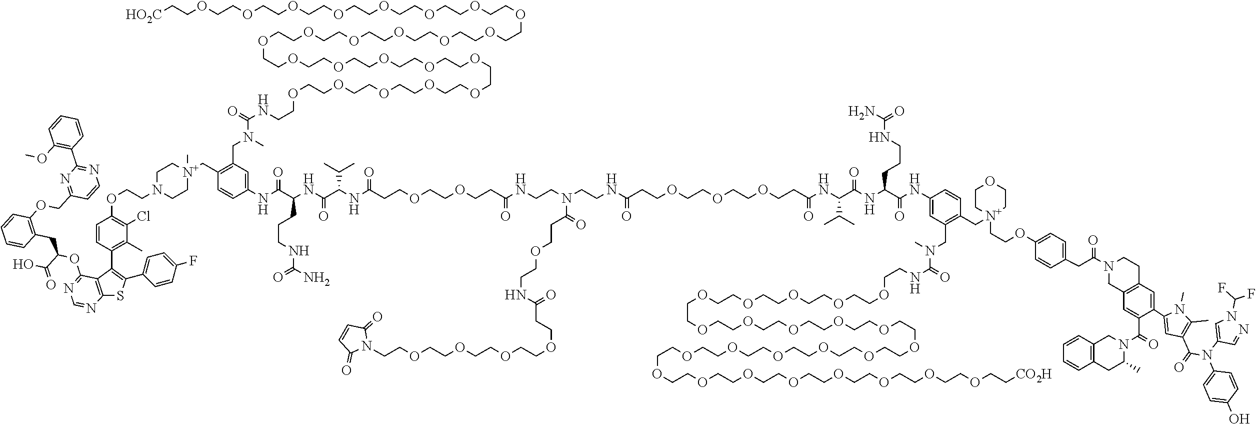 |
| P1-L17- P4 | 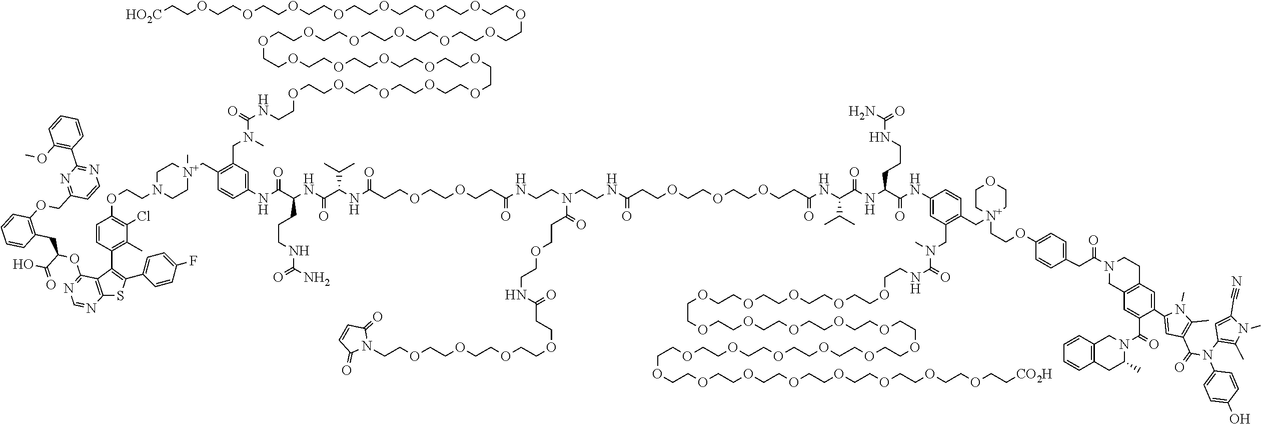 |
| P1-L18- P2 | 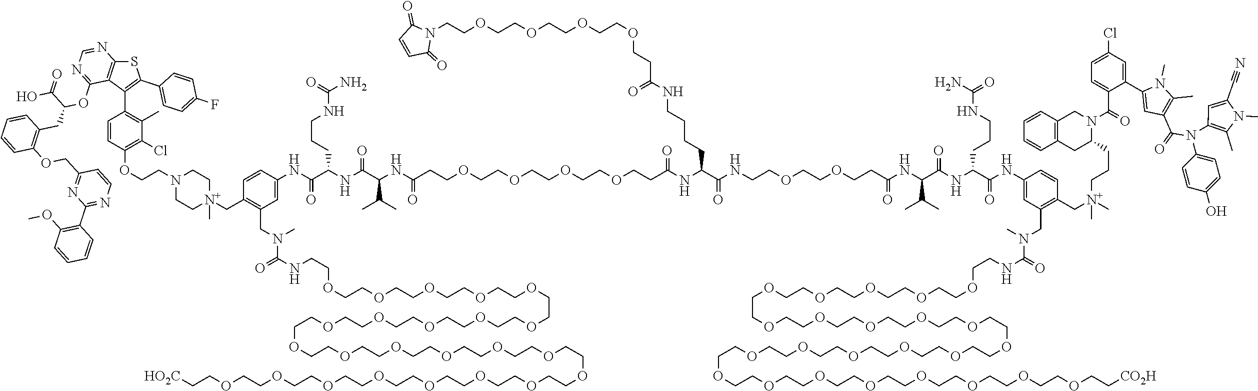 |
| P1-L19- P2 | 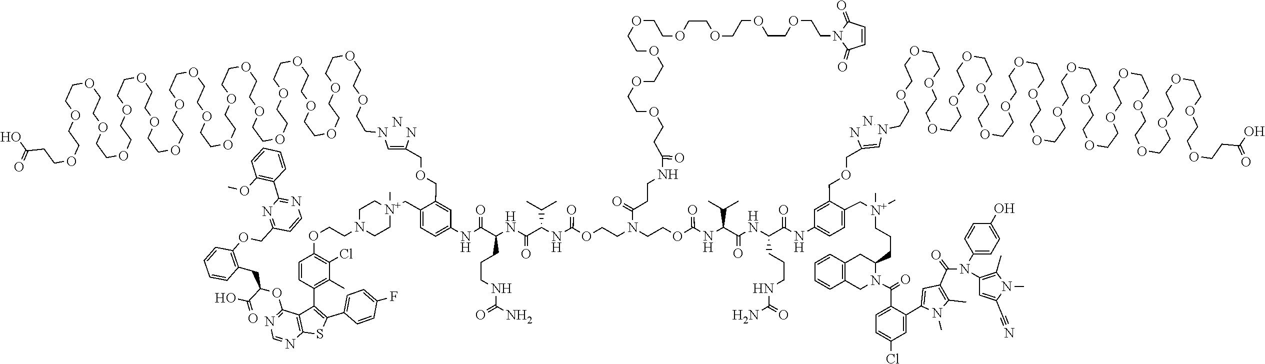 |
| P1-L19- P6 | 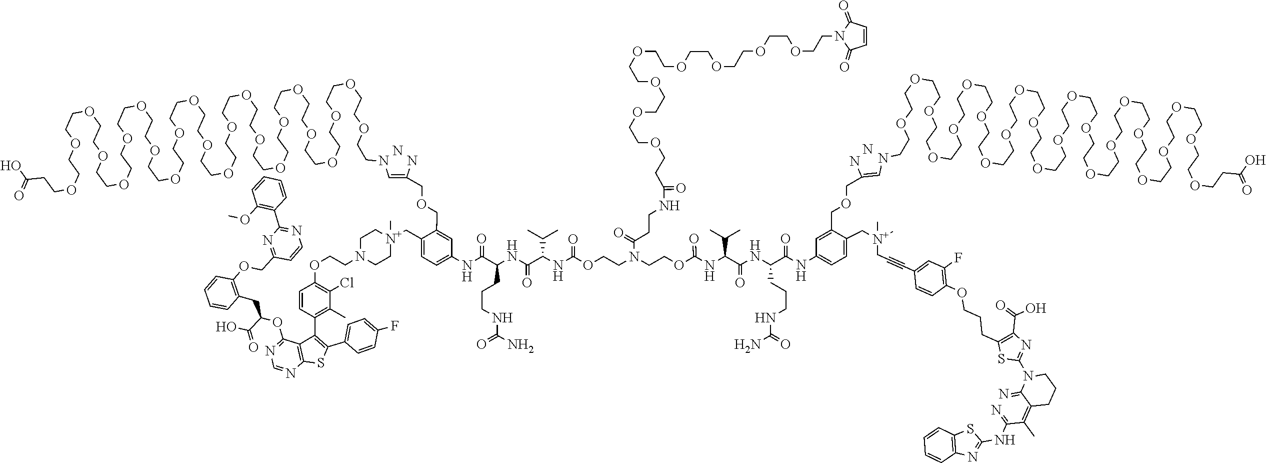 |
| P1-L20- P1 | 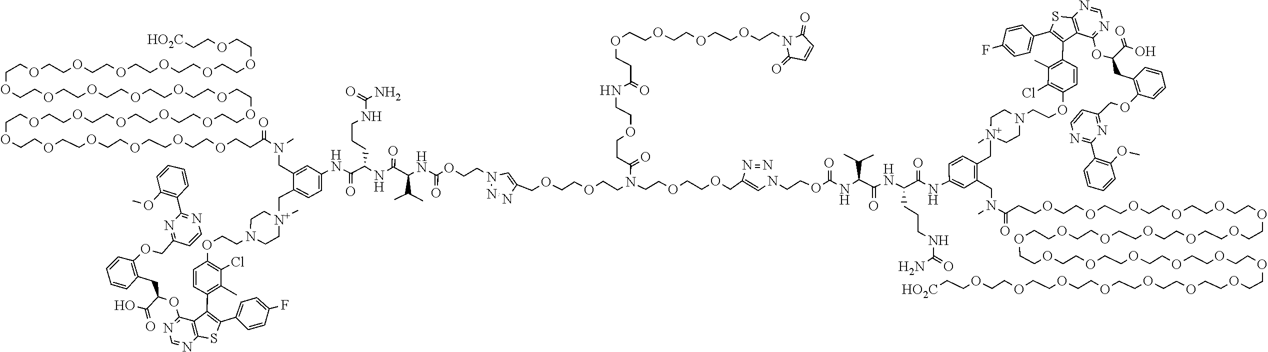 |
| P1-L21- P1 | 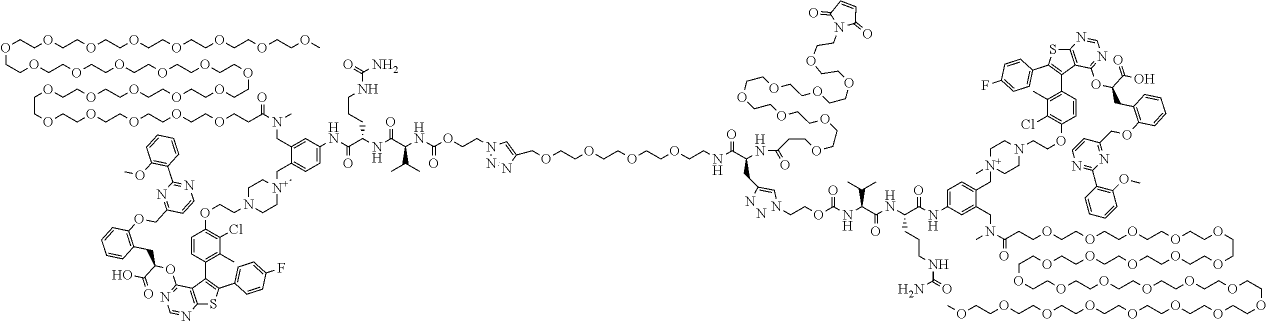 |
| P1-L22- P1 | 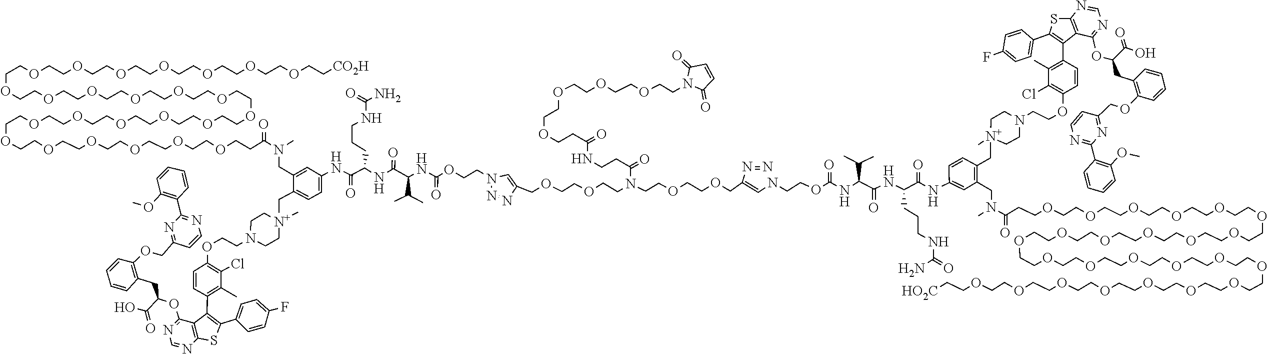 |
| P1-L23- P1 | 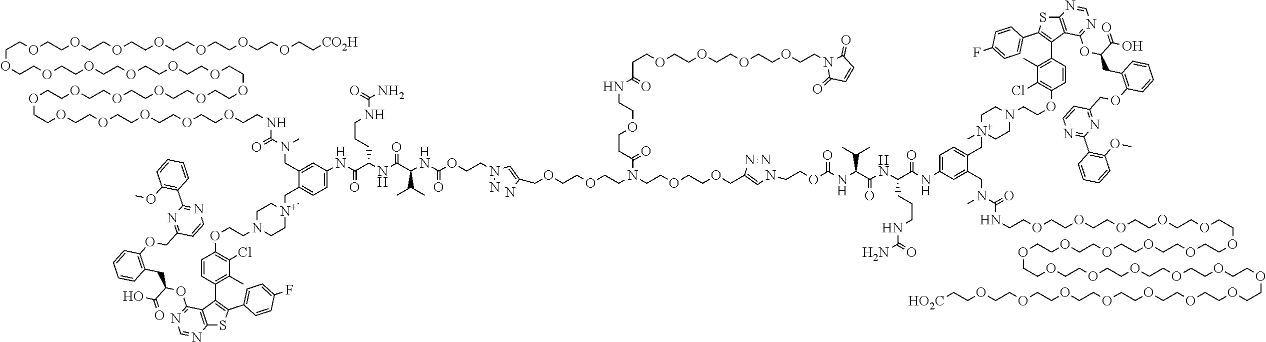 |
| P1-L24- P1 | 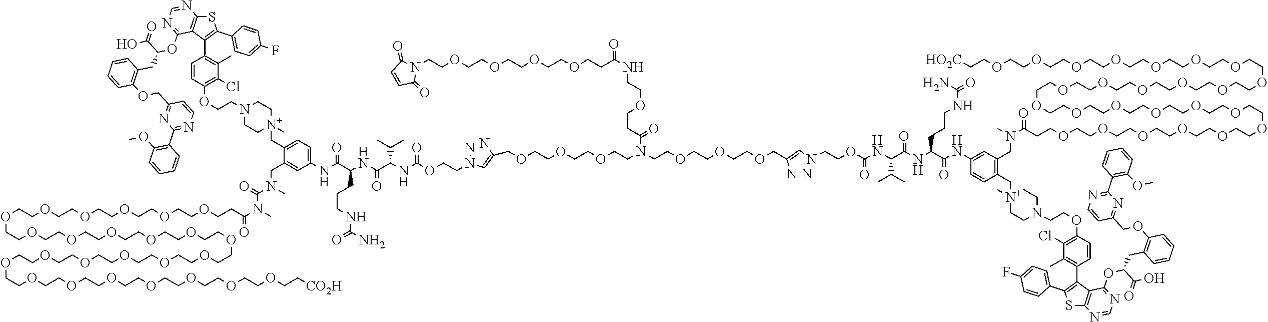 |
| P1-L25- P1 | 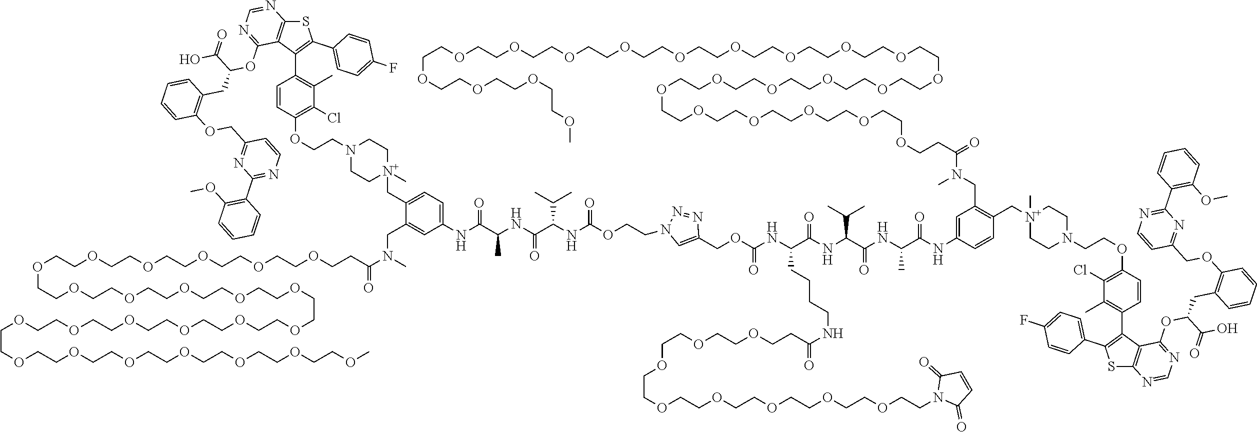 |
| P1-L26- P1 | 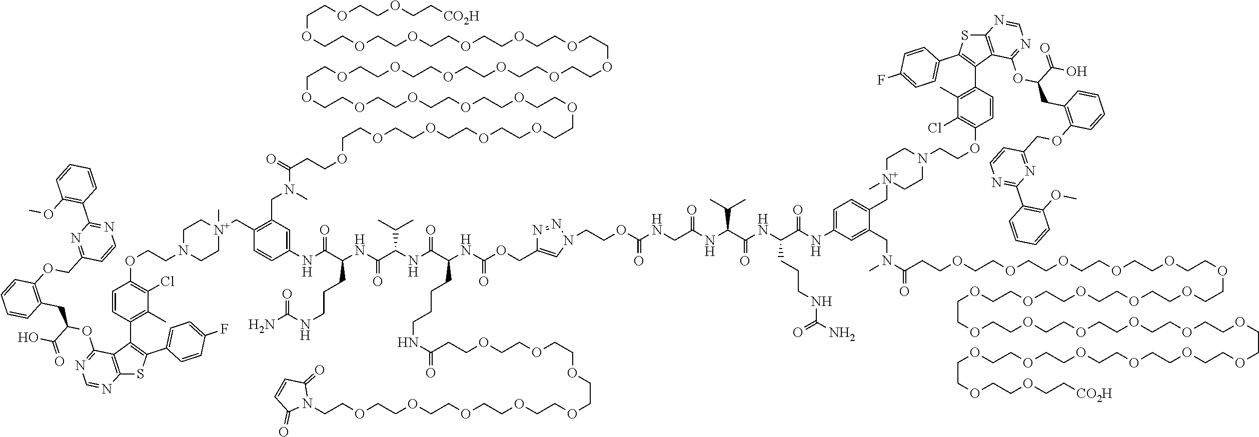 |
| P1-L27- P1 | 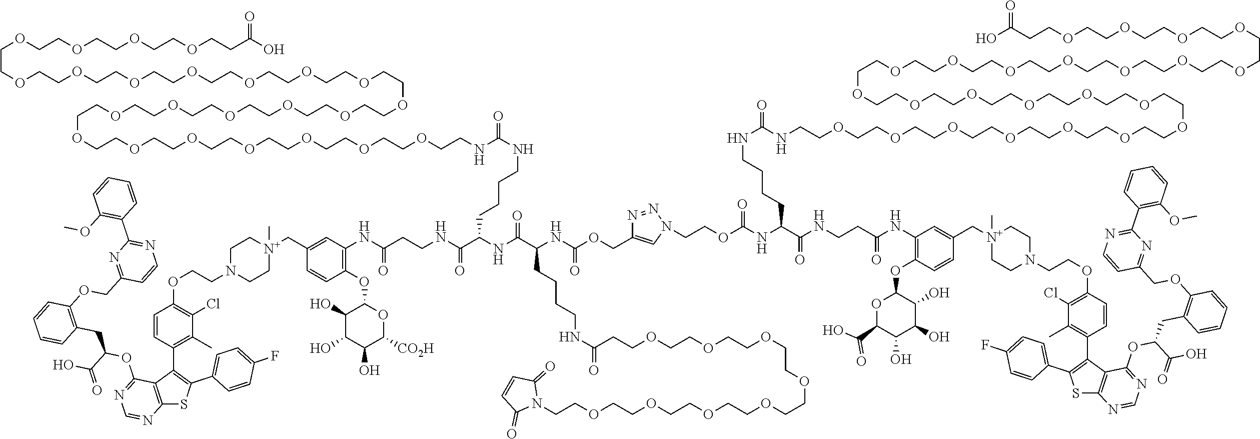 |
| P1-L28- P1 | 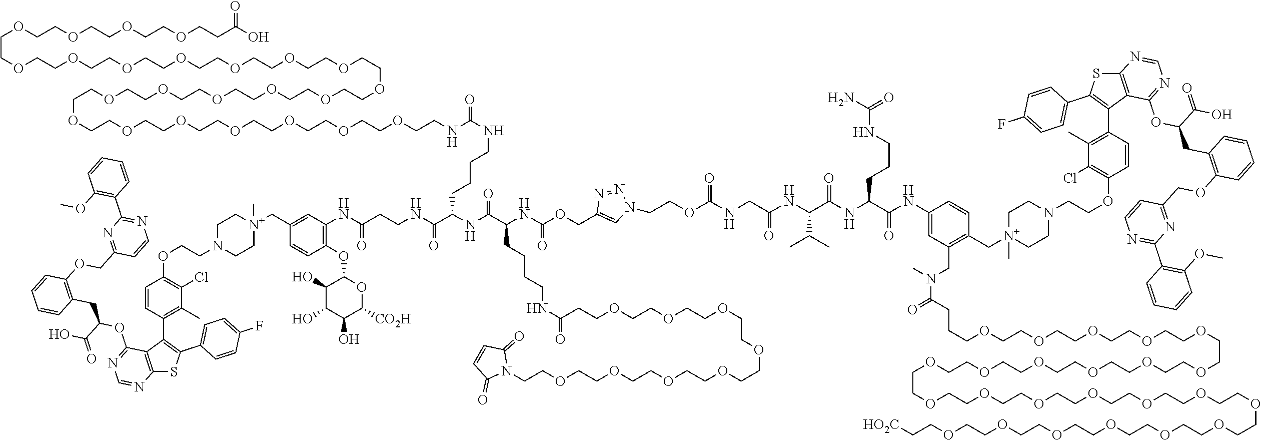 |
| P1-L29- P2 | 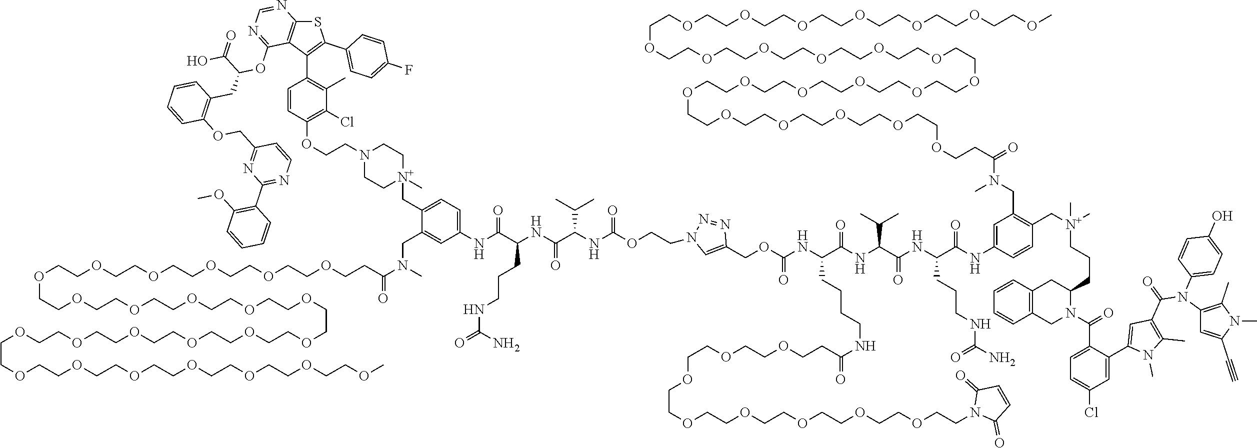 |
| P2-L29- P1 | 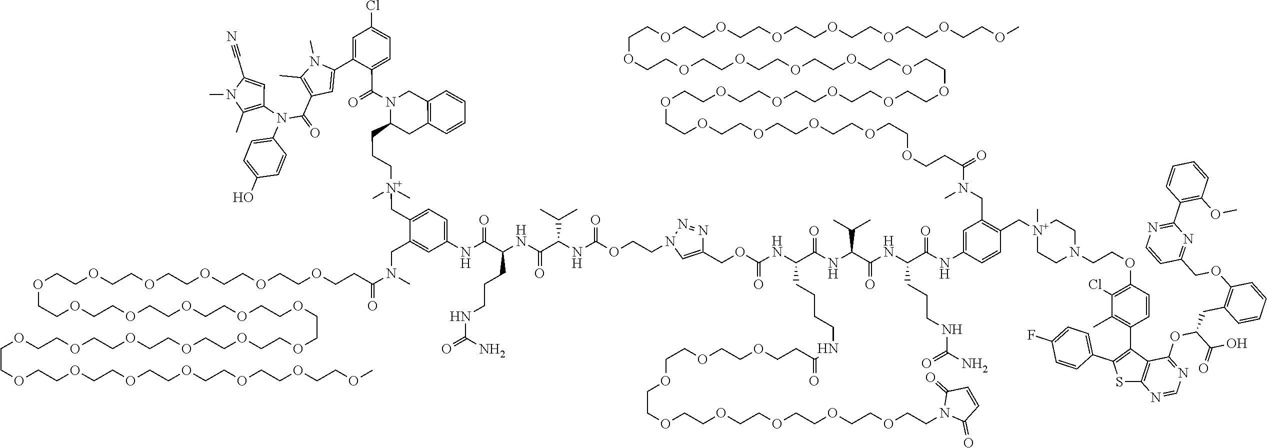 |
| P1-L30- P2 | 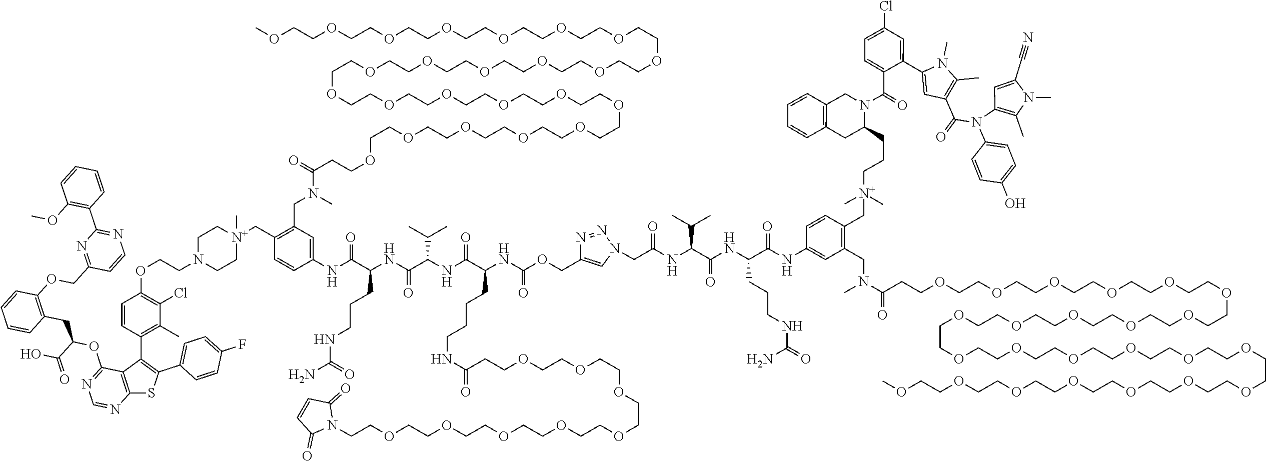 |
| P1-L31- P2 | 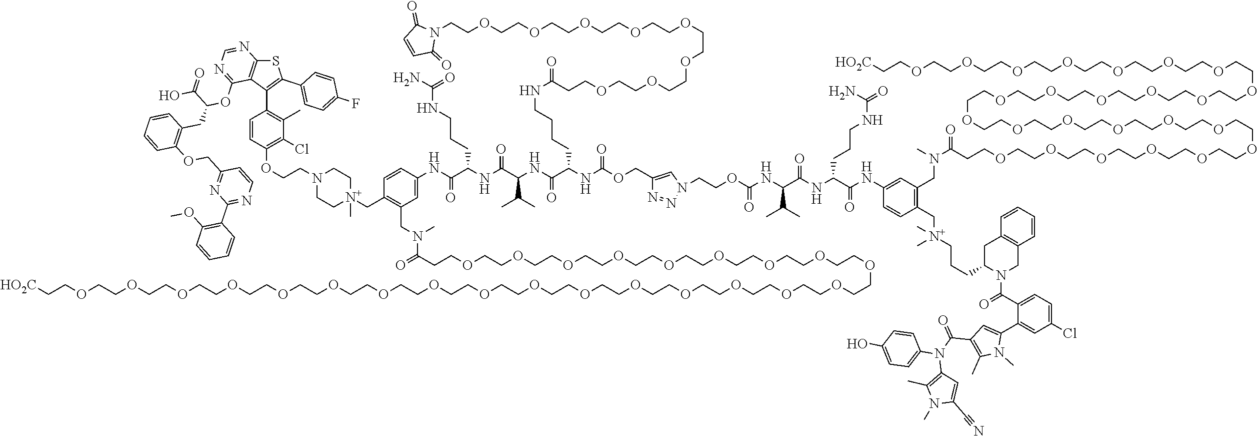 |
| P1-L32- P2 | 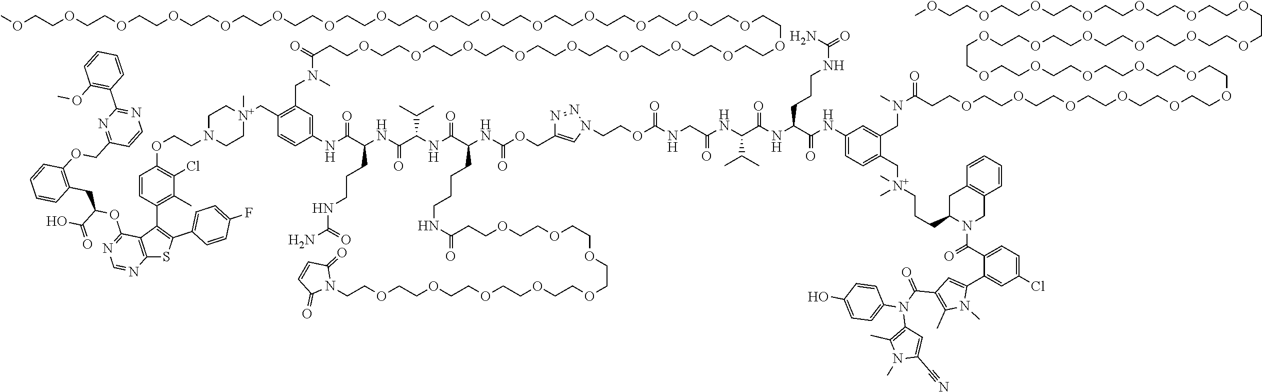 |
| P1-L33- P1 | 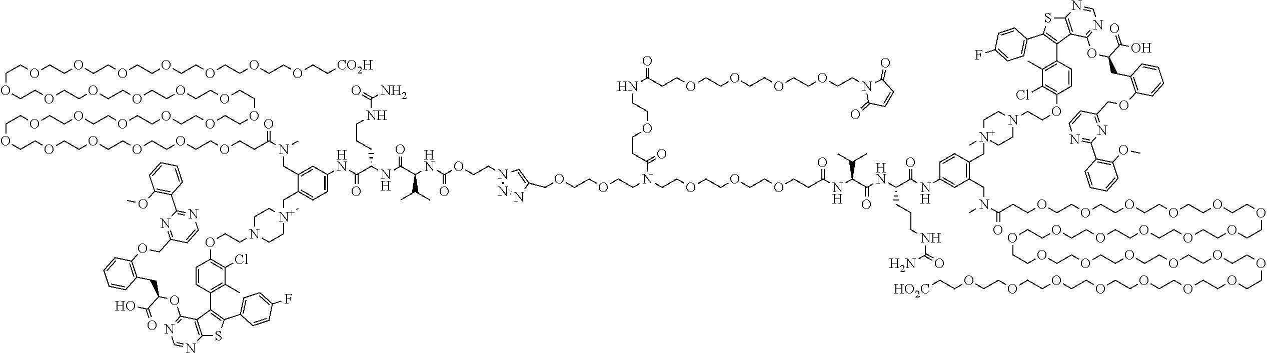 |
| P1-L34- P1 | 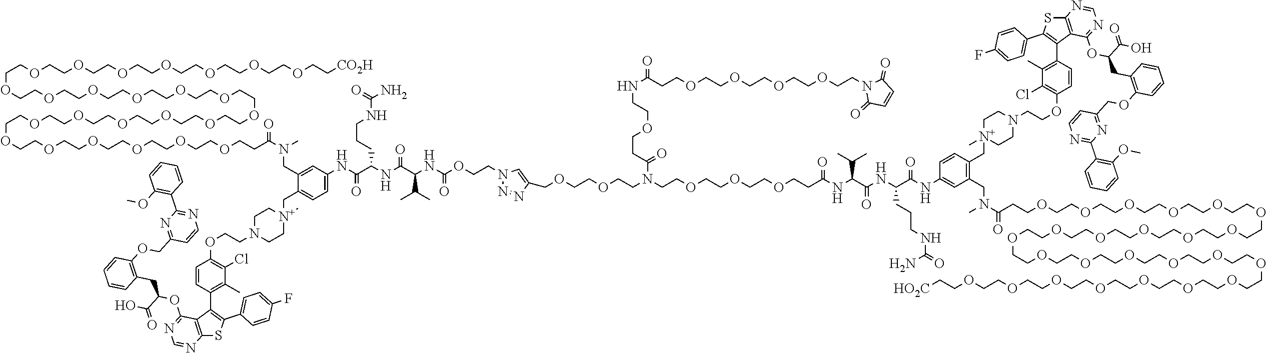 |
| P1-L35- P1 | 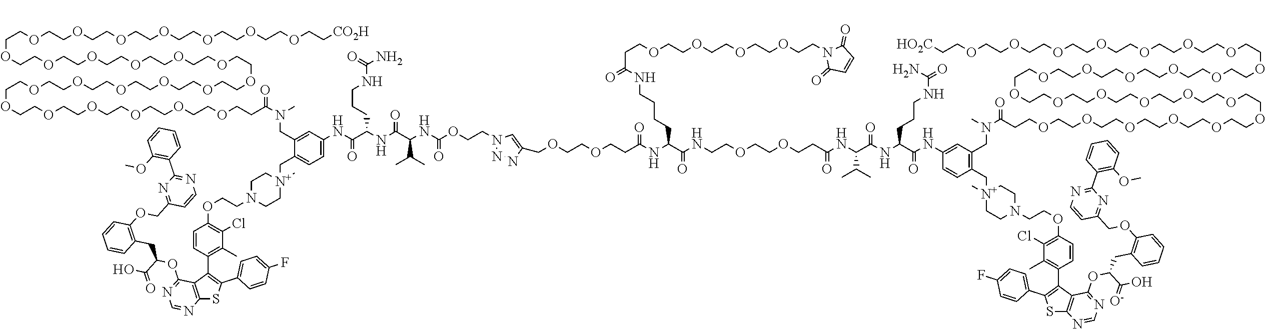 |
| P1-L36- P1 | 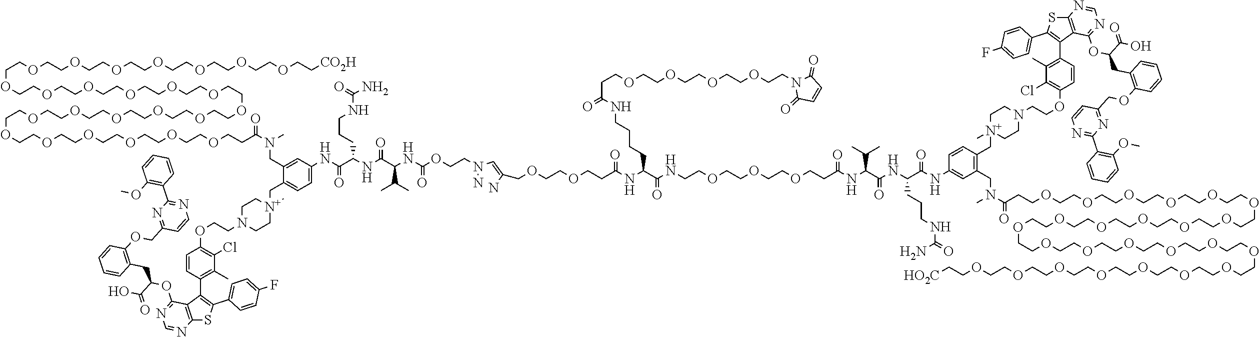 |

In some embodiments, the antibody-drug conjugate has a formula according to any one of the structures shown in Table C.

TABLE C
Exemplary ADC Structures

| ADC | Structure |
|---|---|
| Ab-P1- L1-P1 | 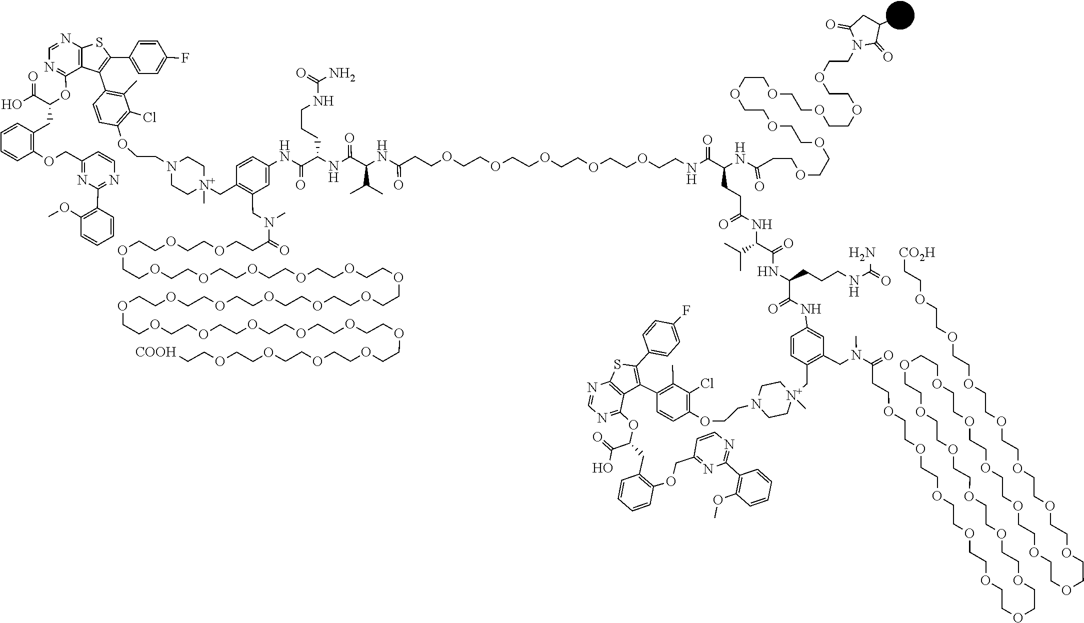 |
| Ab-P1- L2-P1 | 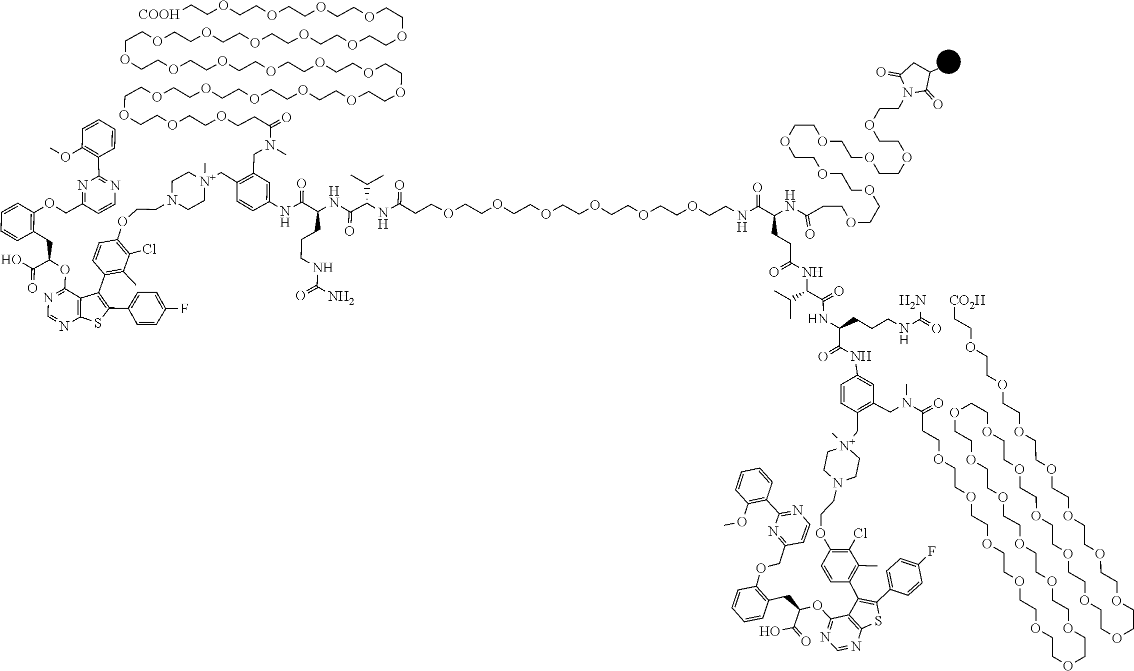 |
| Ab-P1- L3-P1 | 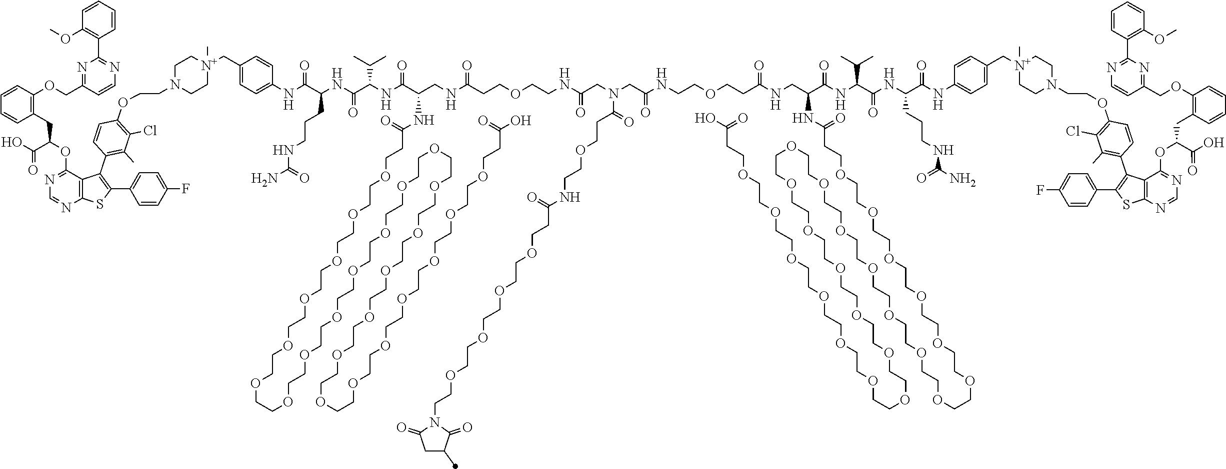 |
| Ab-P1- L4-P1 | 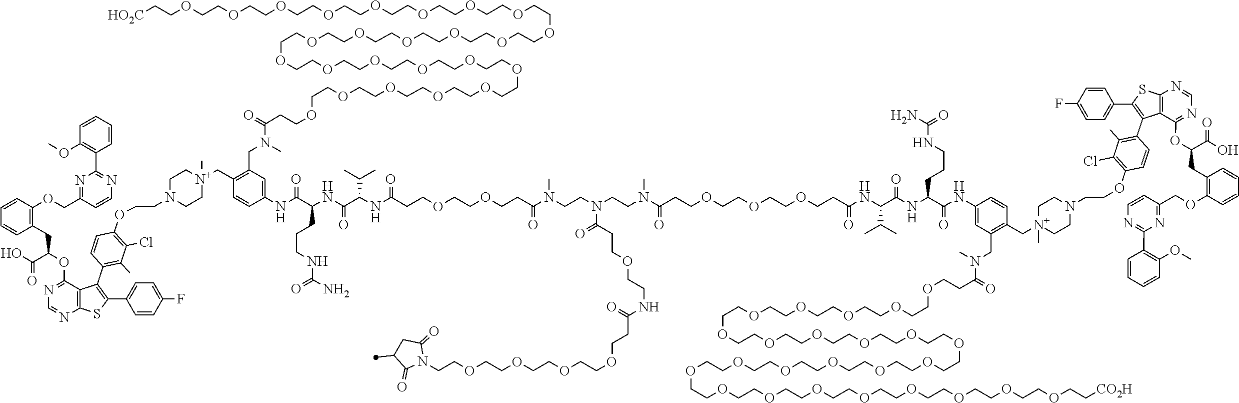 |
| Ab-P1- L5-P1 | 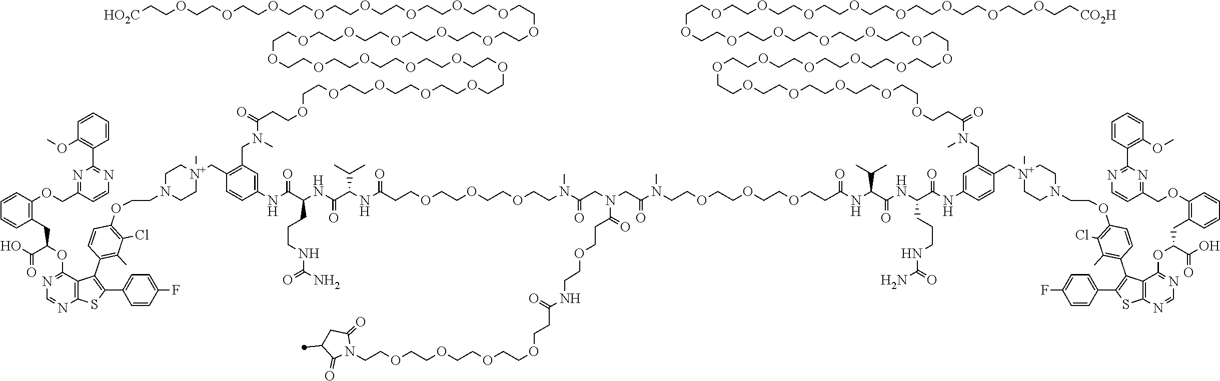 |
| Ab-P1- L6-P1 | 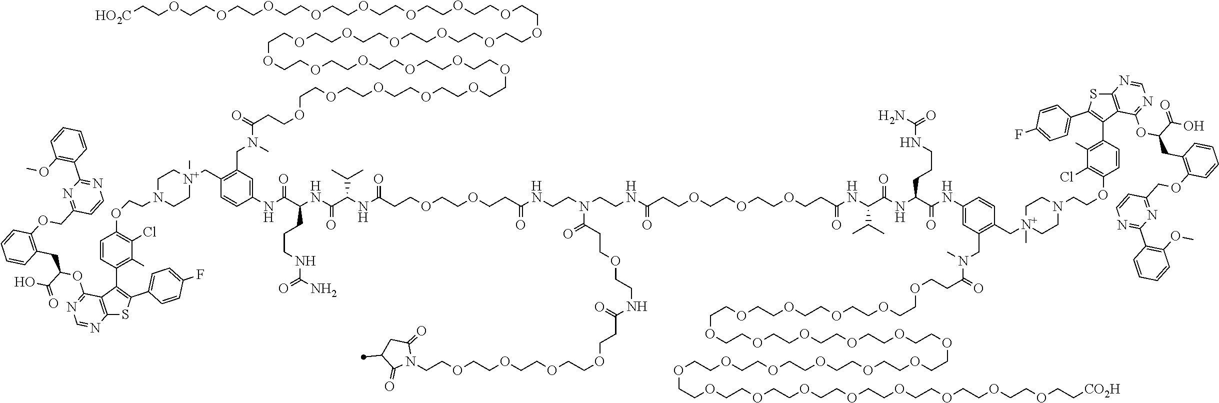 |
| Ab-P1- L7-P1 | 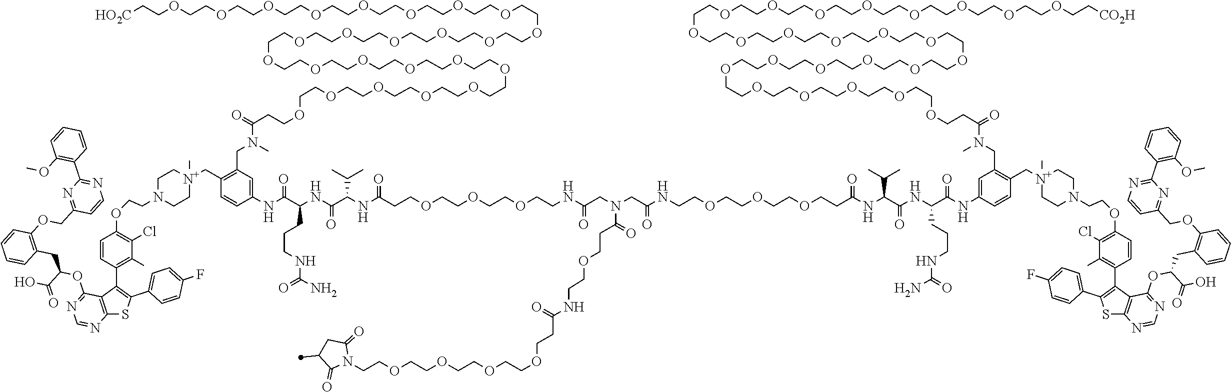 |
| Ab-P1- L8-P1 | 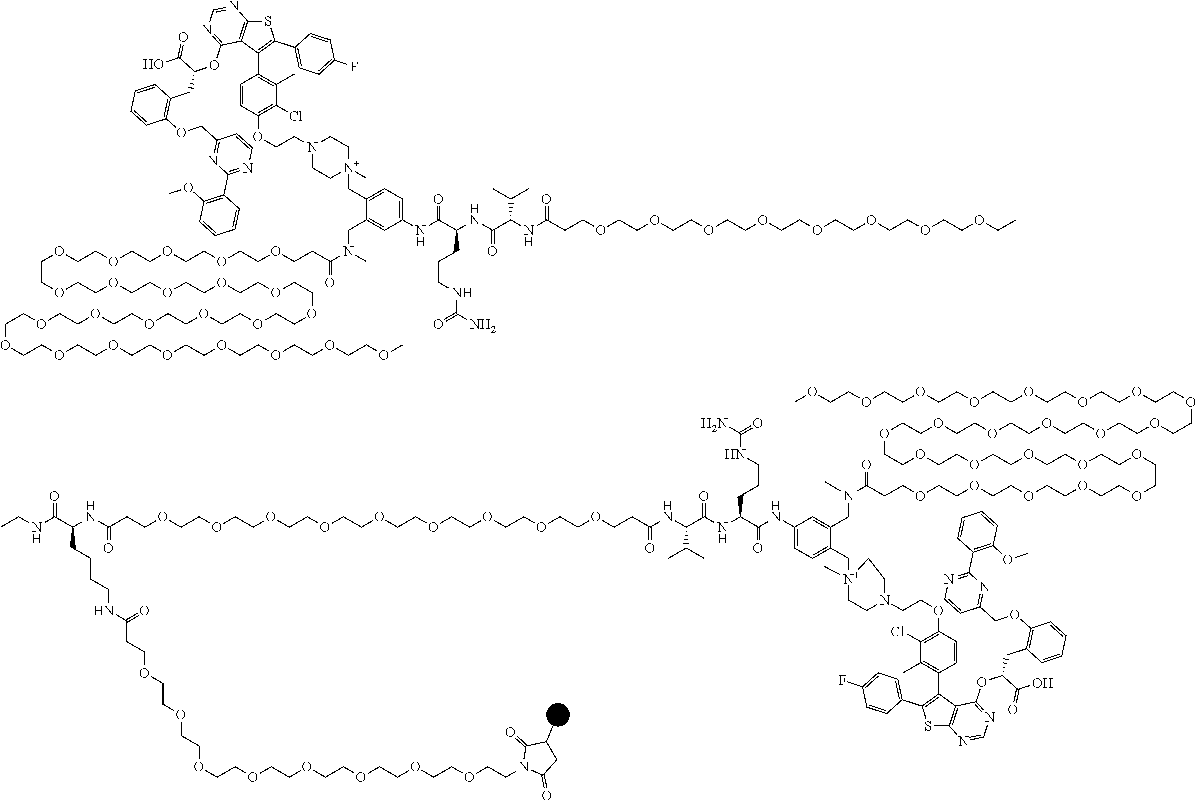 |
| Ab-P1- L9-P1 | 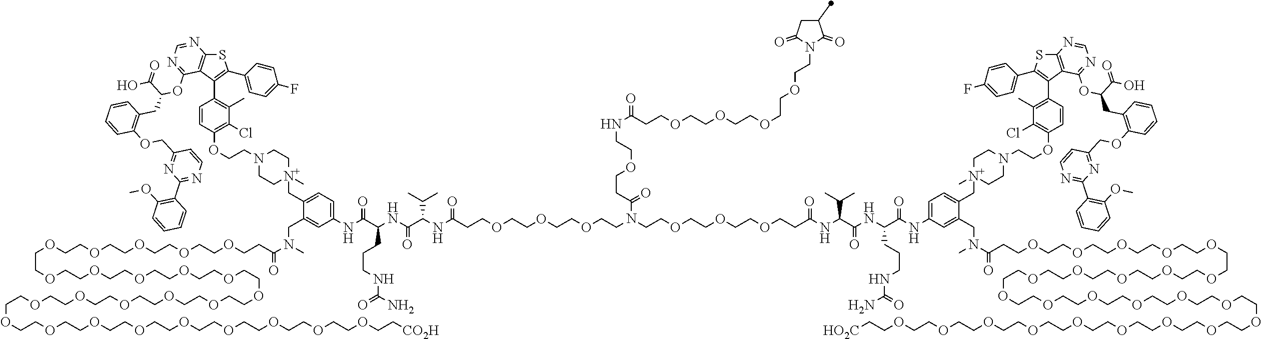 |
| Ab-P1- L10-P1 | 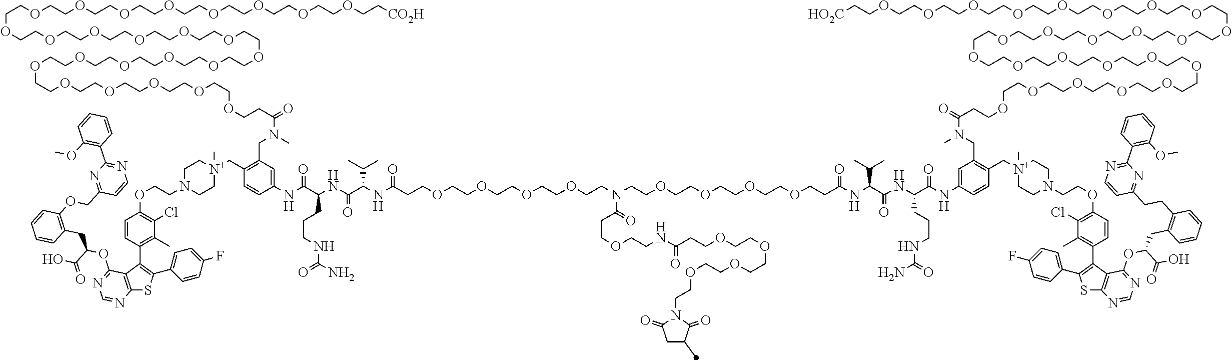 |
| Ab-P1- L11-P1 | 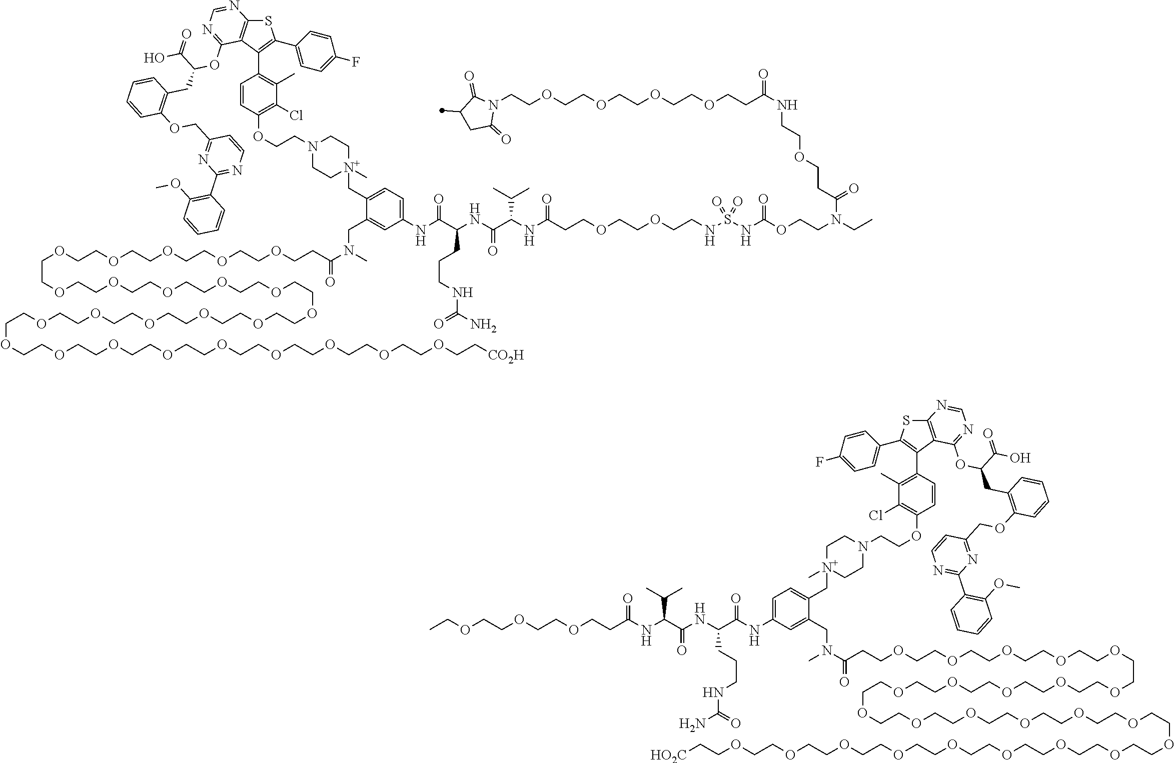 |
| Ab-P1- L12-P2 | 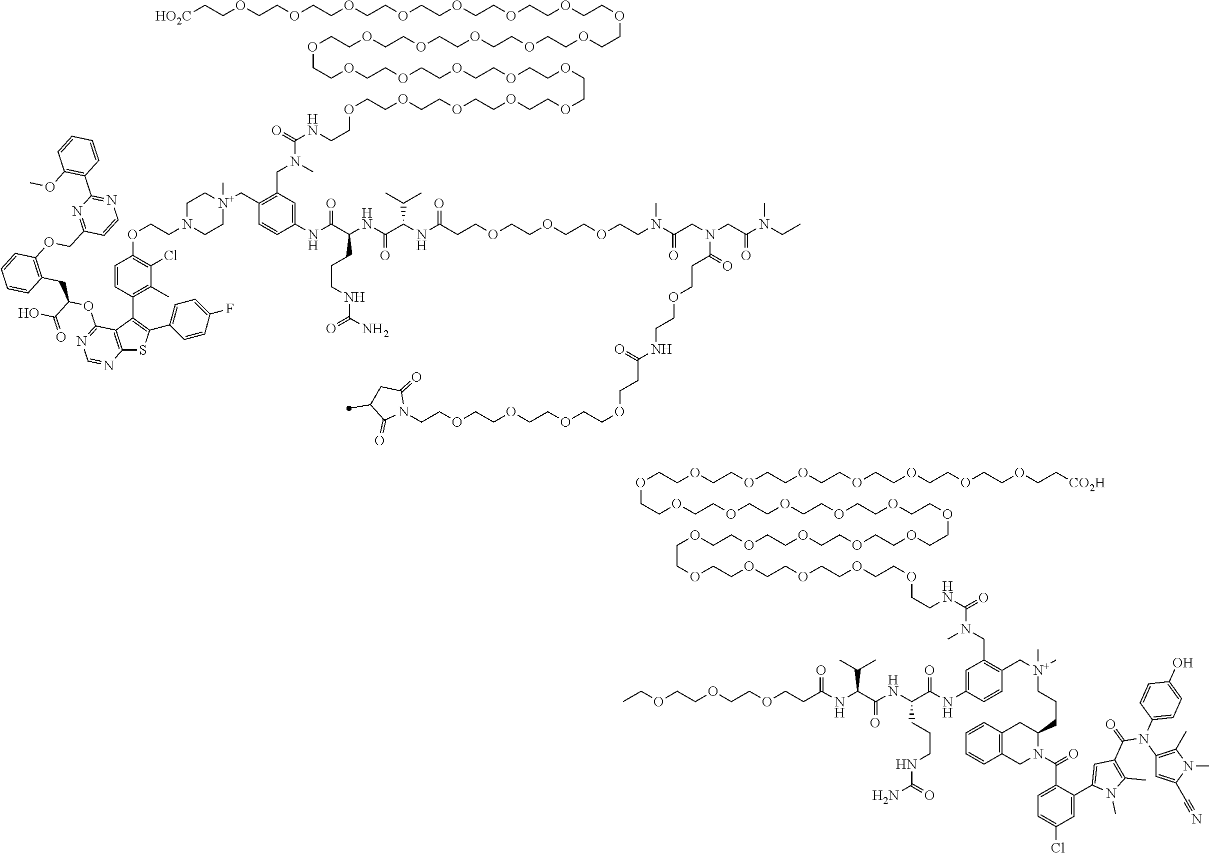 |
| Ab-P1- L12-P3 | 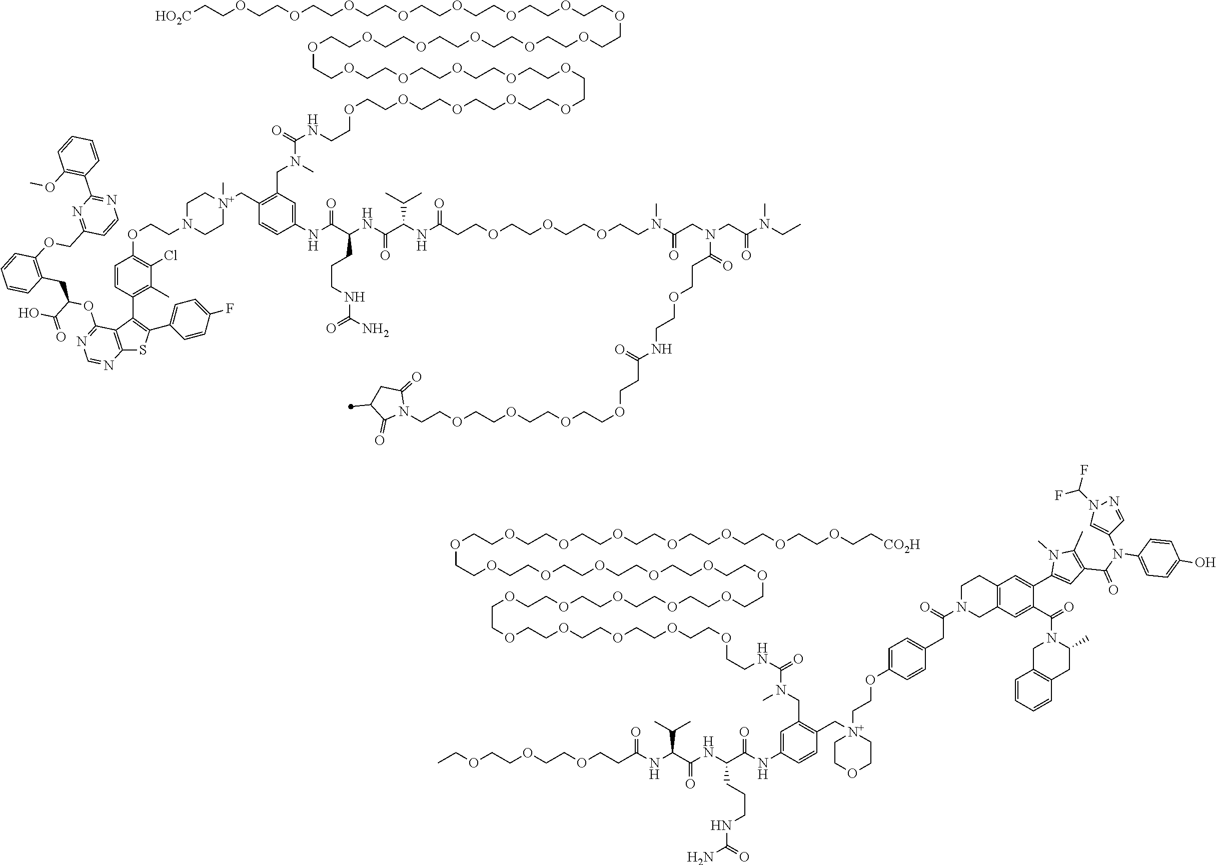 |
| P1- L12-P4 | 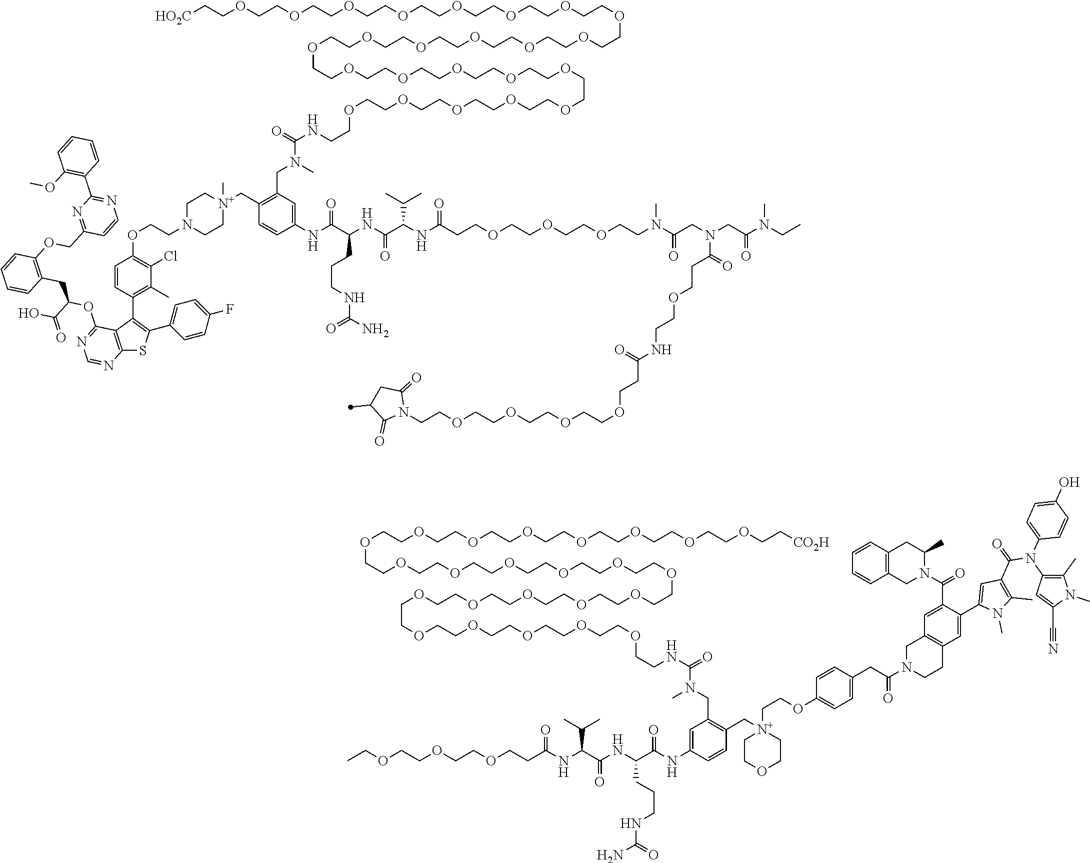 |
| Ab-P5- L12-P5 | 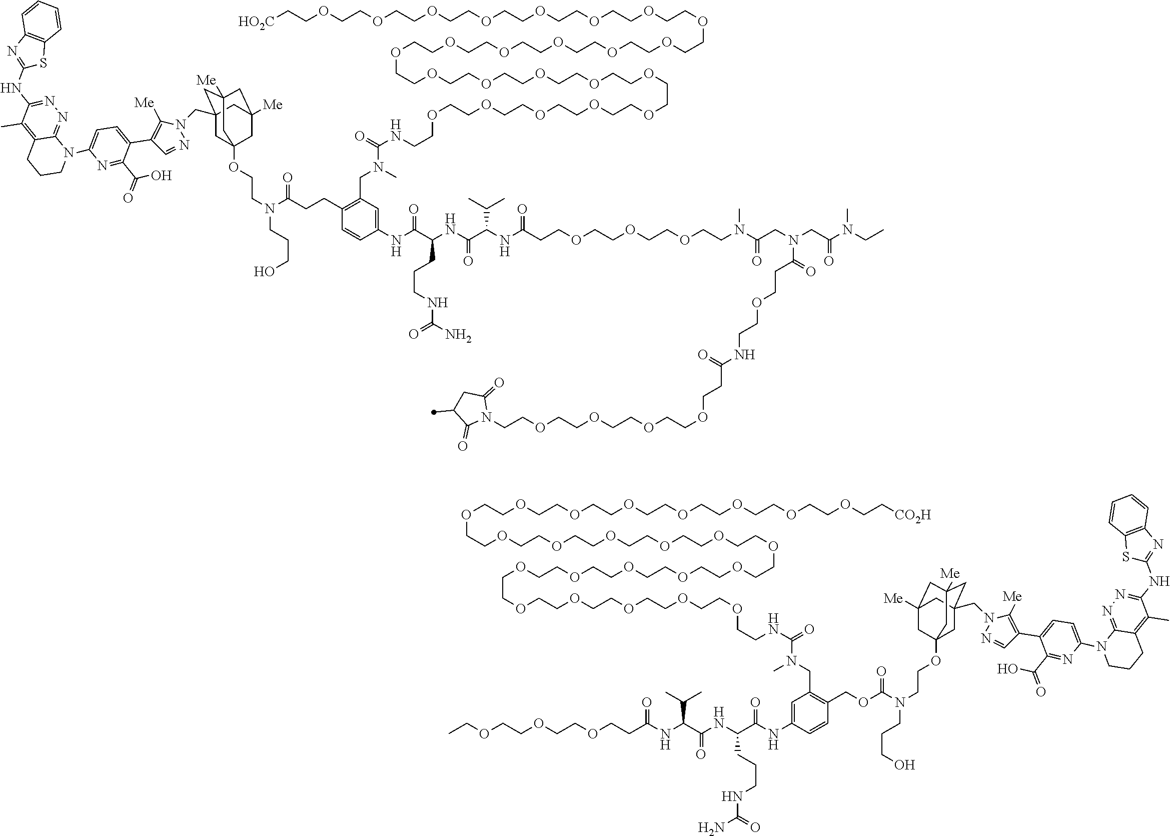 |
| Ab-P5- L12-P7 | 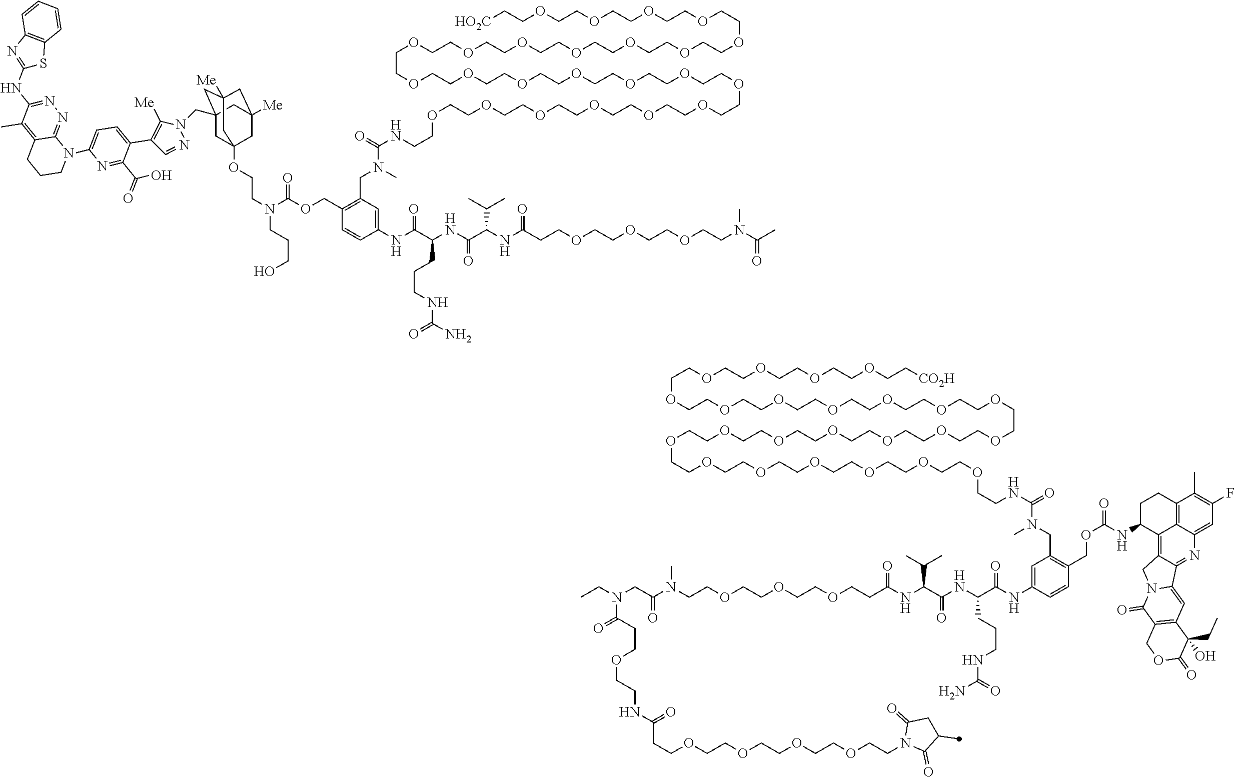 |
| Ab-P5- L12-P4 | 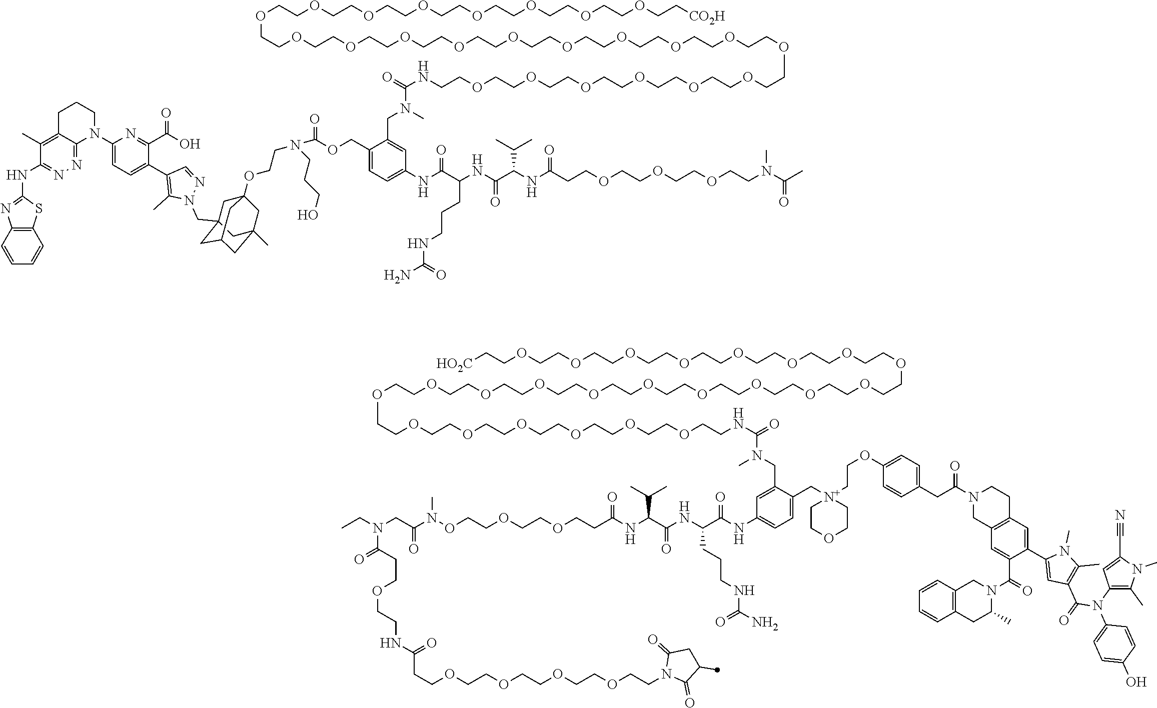 |
| Ab-P5- L12-P8 | 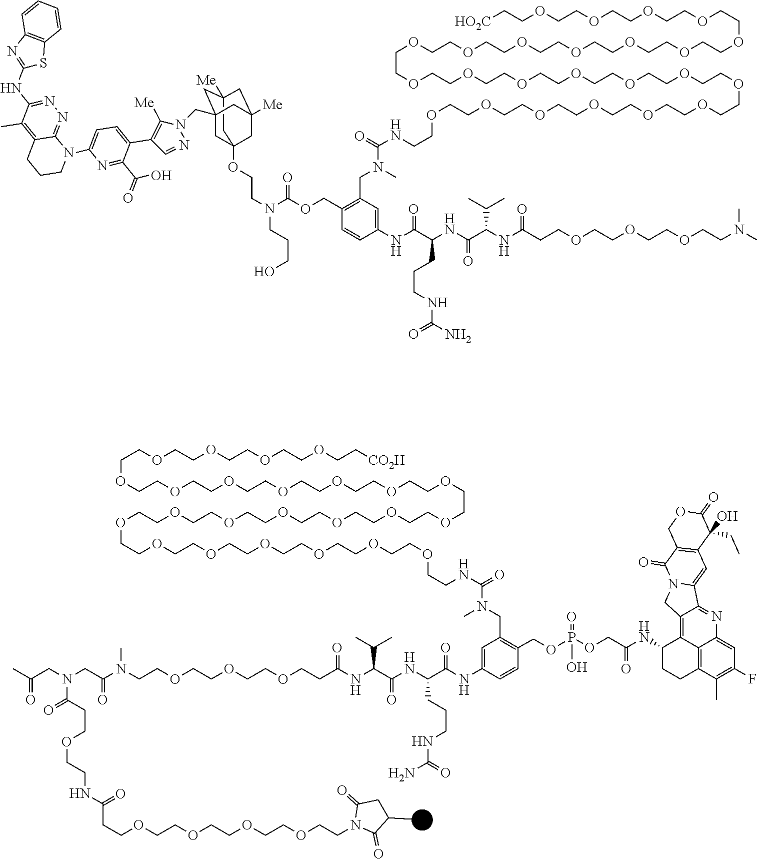 |
| Ab-P5- L37-P8 | 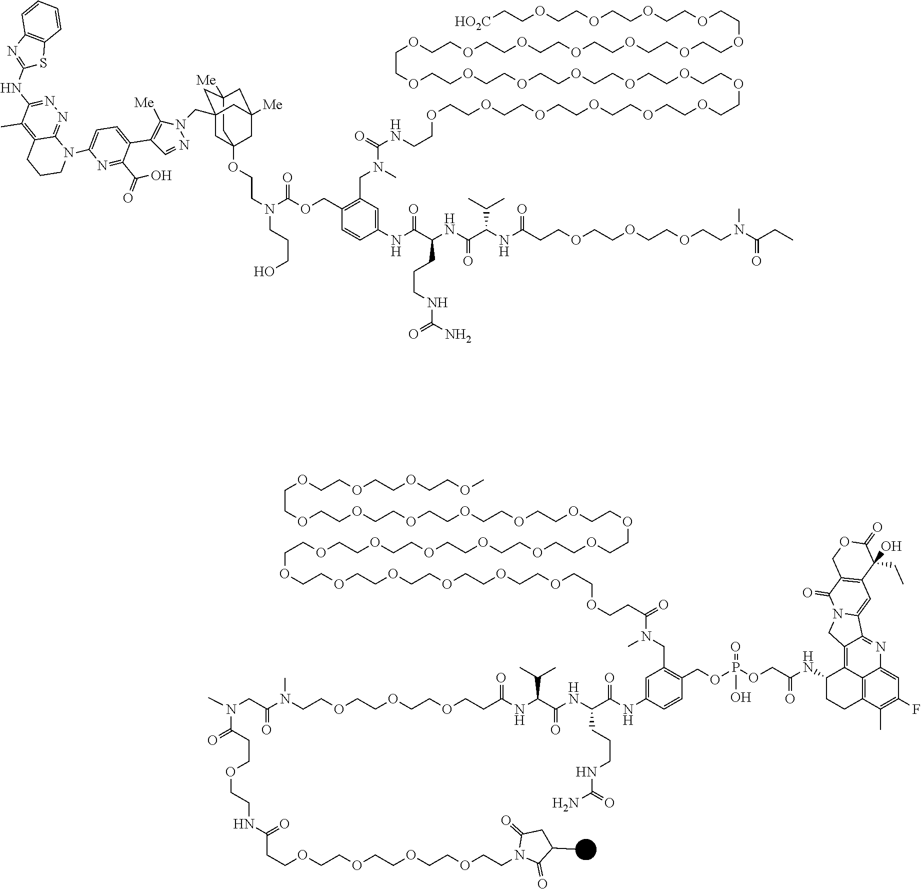 |
| Ab-P2- L13-P1 | 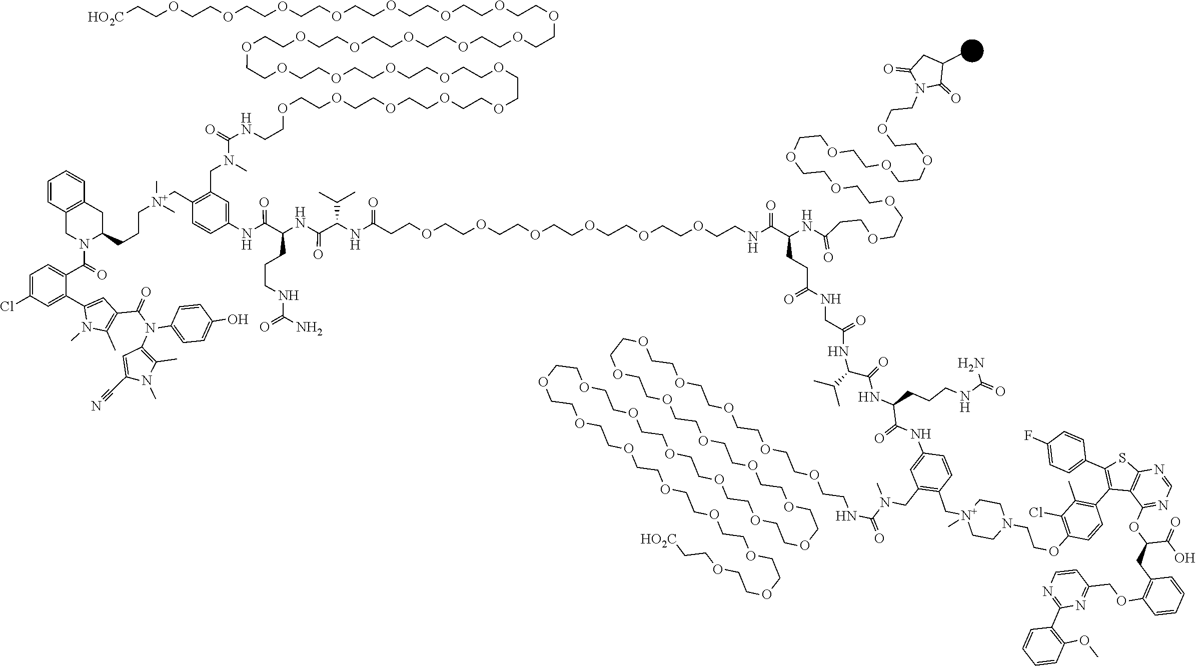 |
| Ab-P2- L14-P1 | 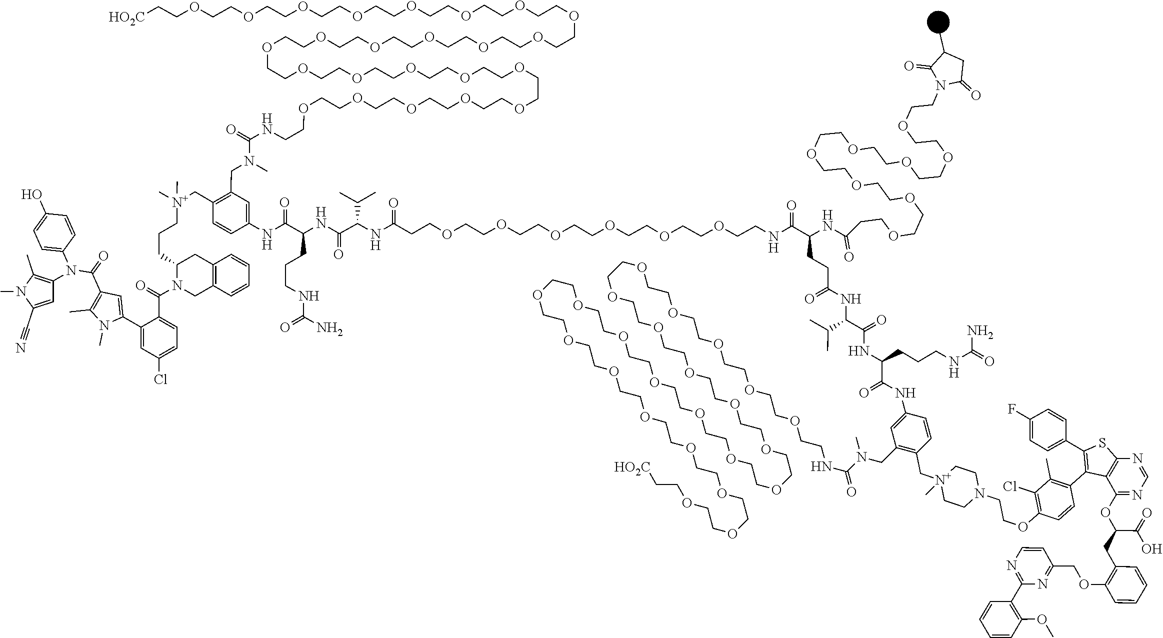 |
| Ab-P1- L15-P2 | 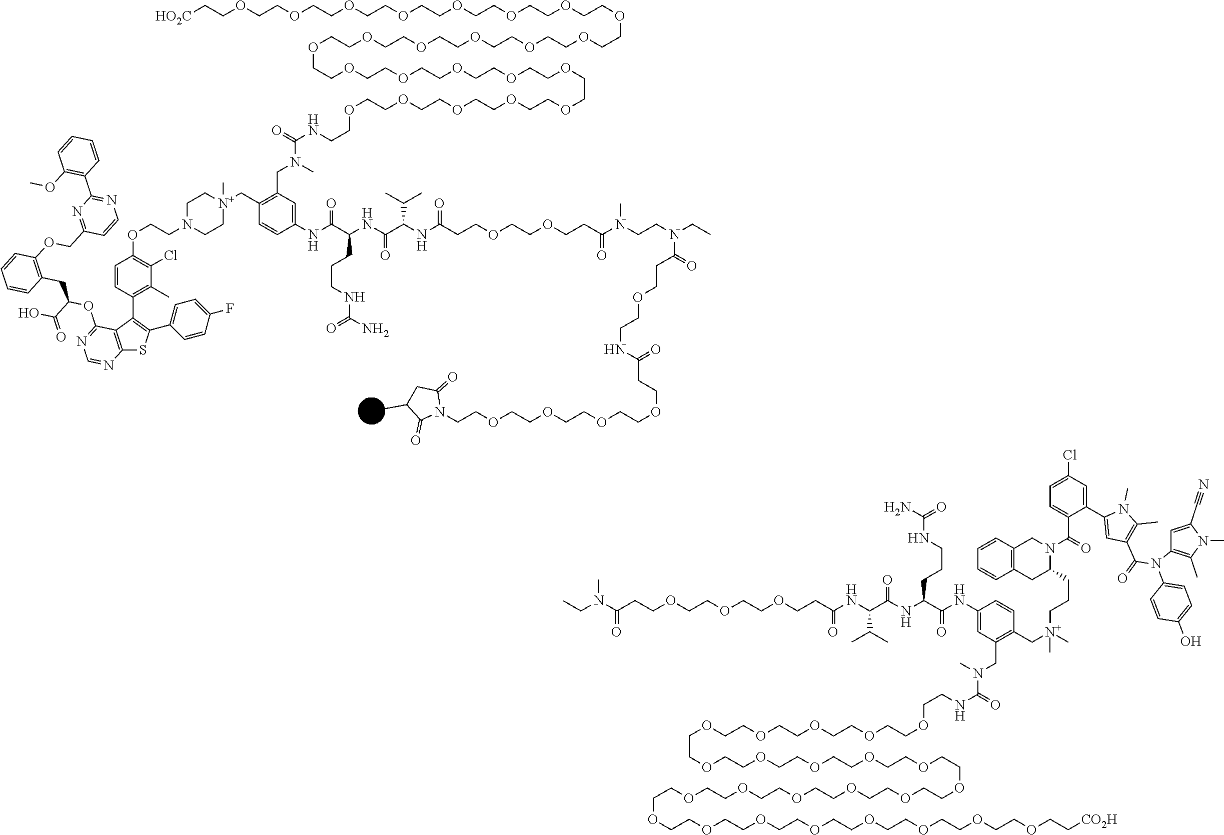 |
| Ab-P1- L16-P2 | 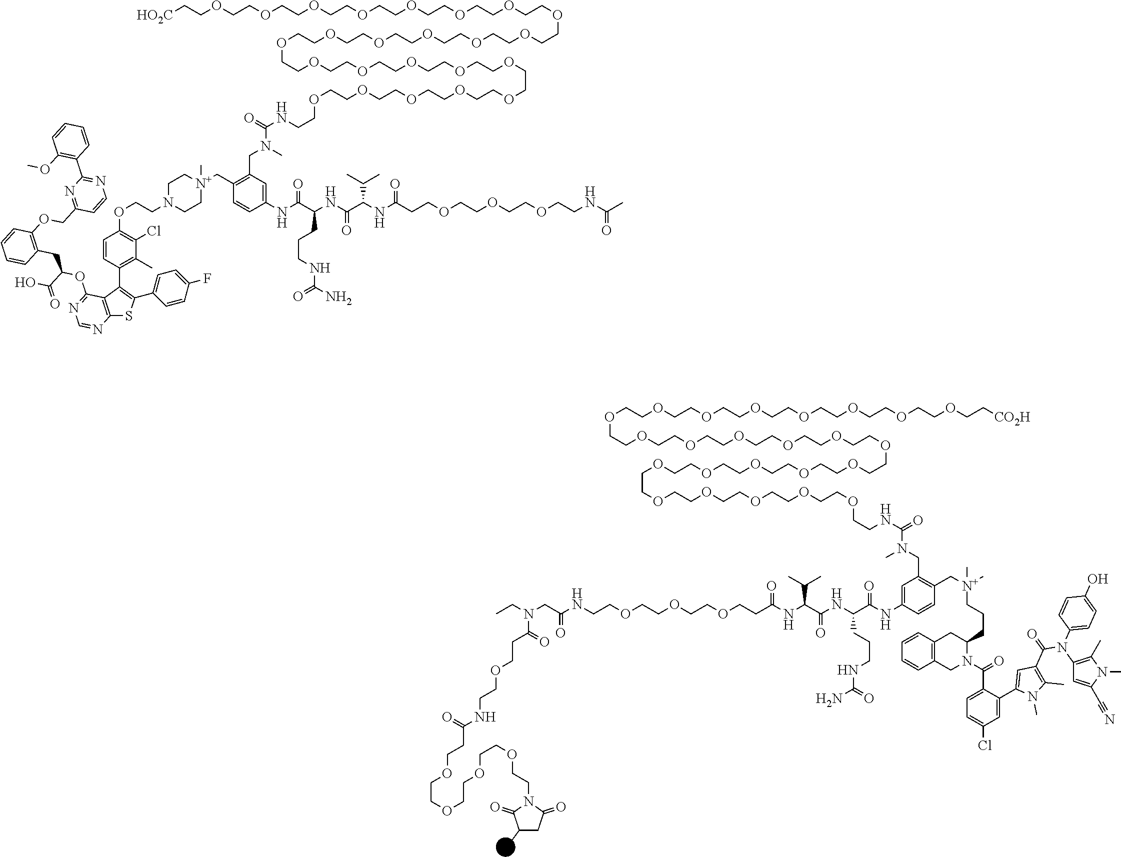 |
| Ab-P1- L17-P2 | 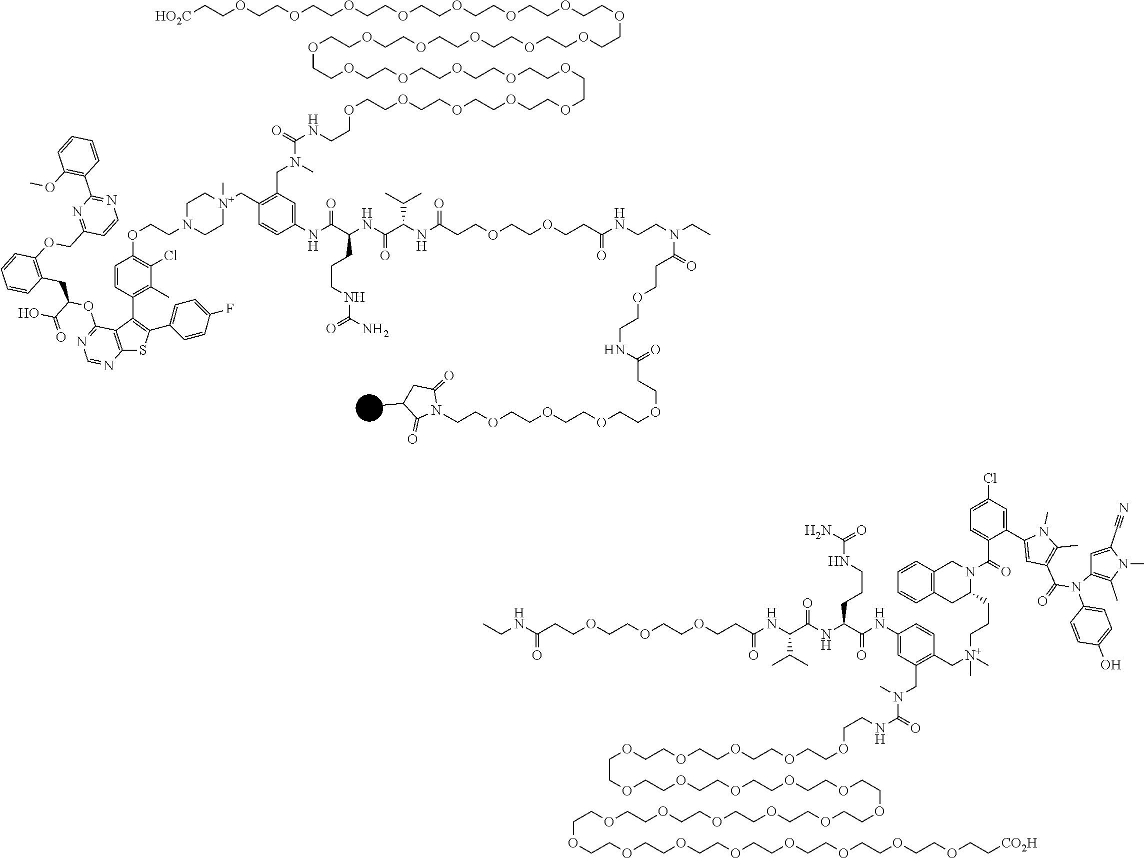 |
| Ab-P1- L17-P3 | 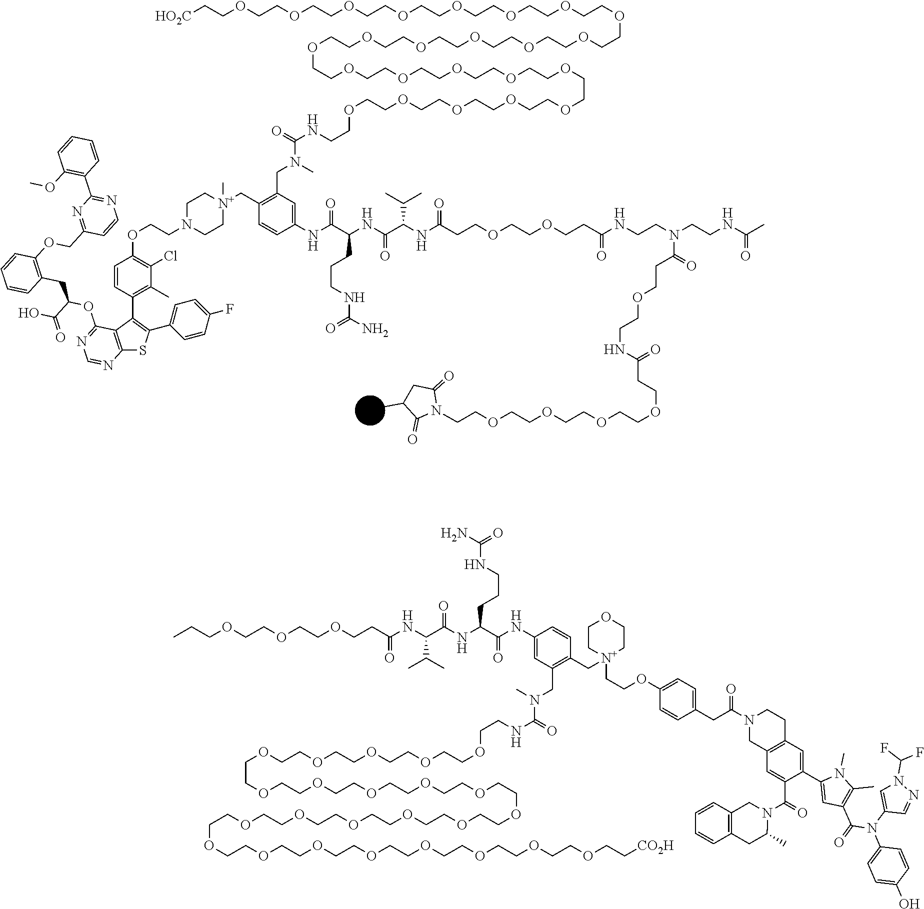 |
| Ab-P1- L17-P4 | 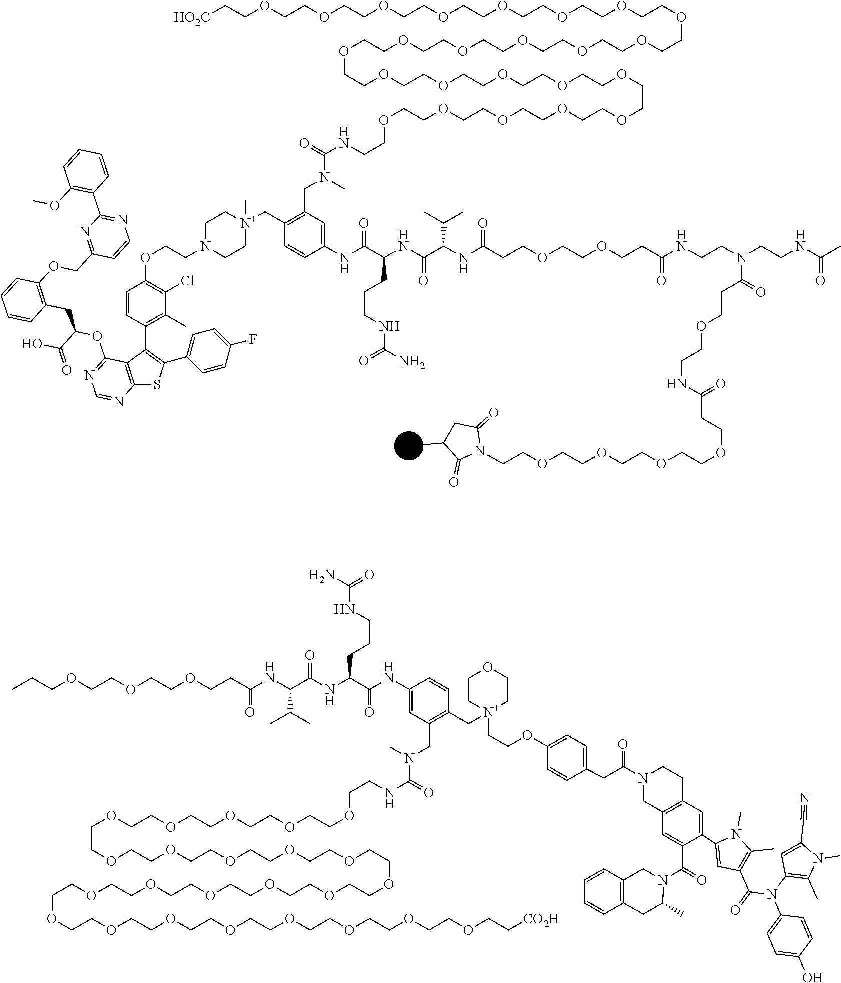 |
| Ab-P1- L18-P2 | 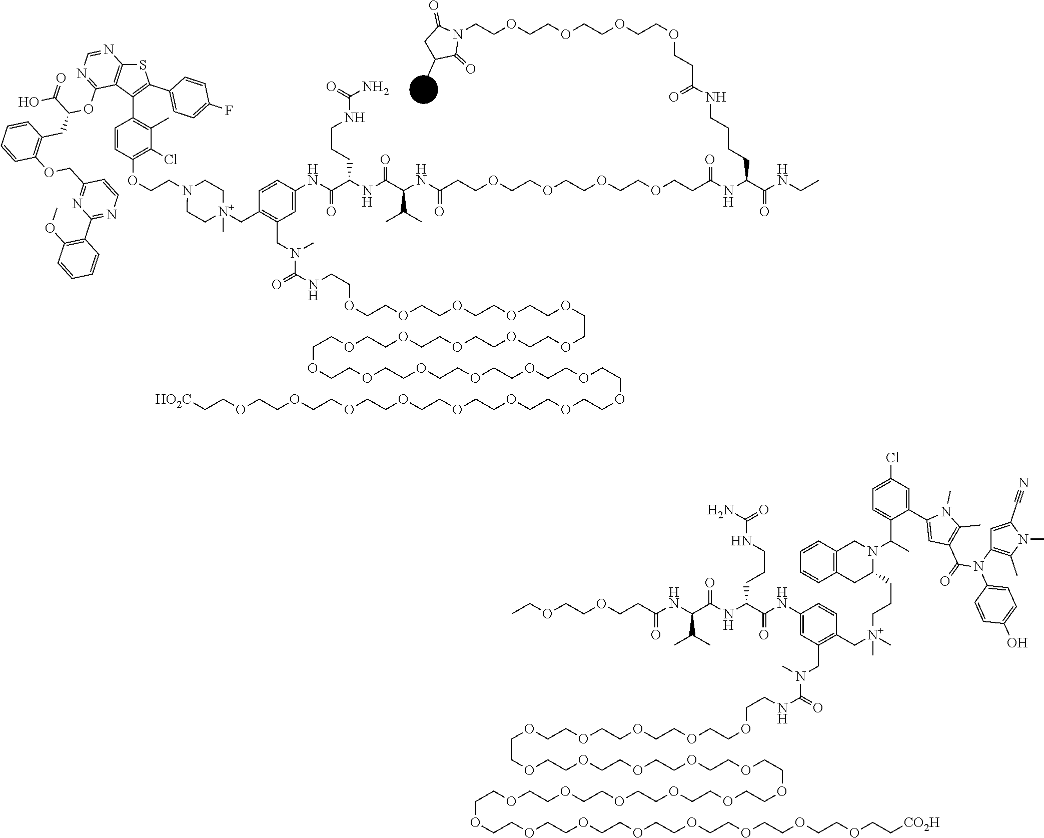 |
| Ab-P1- L19-P2 | 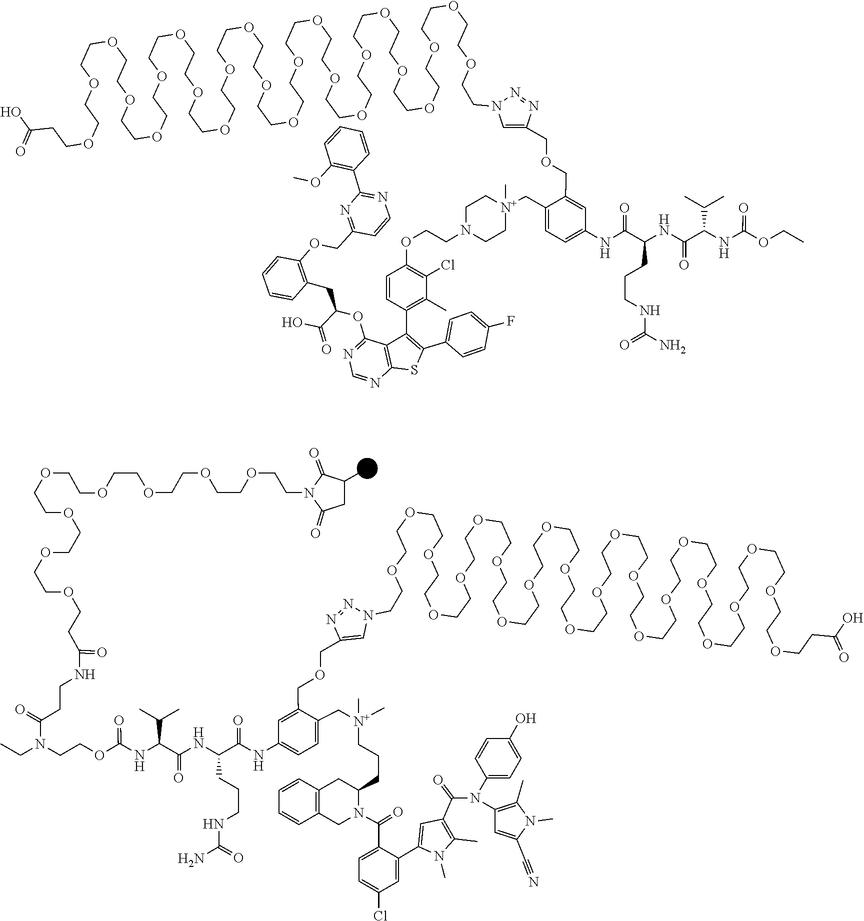 |
| Ab-P1- L19-P6 | 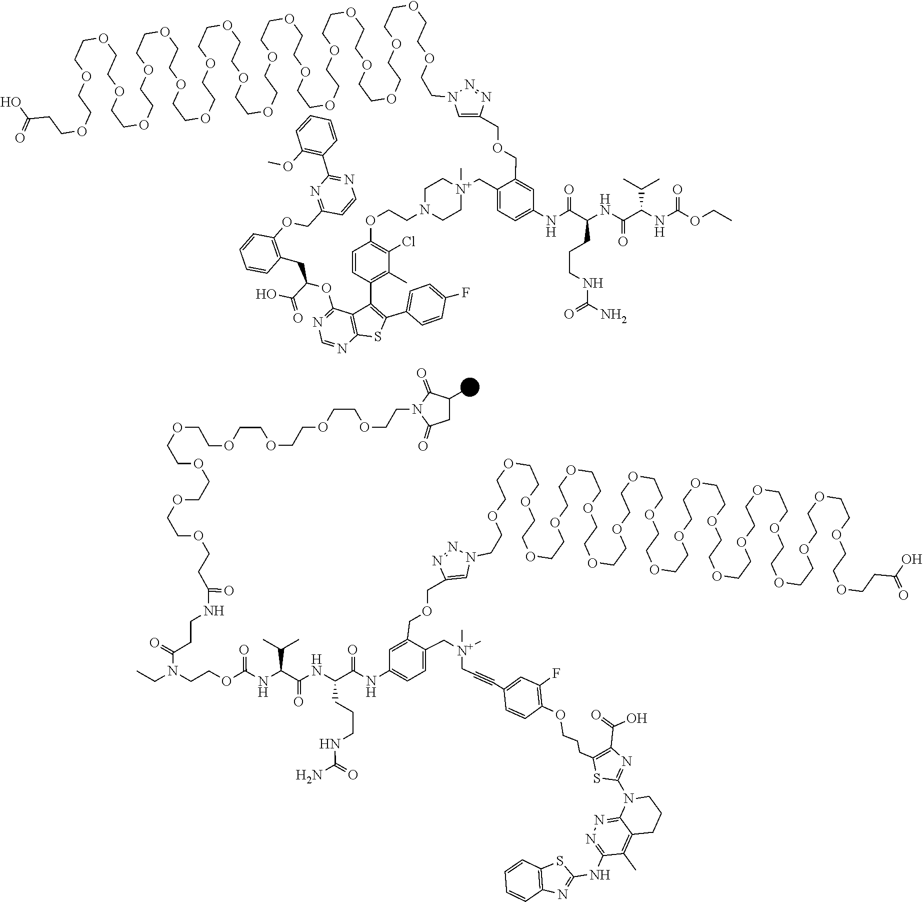 |
| Ab-P1- L20-P1 | 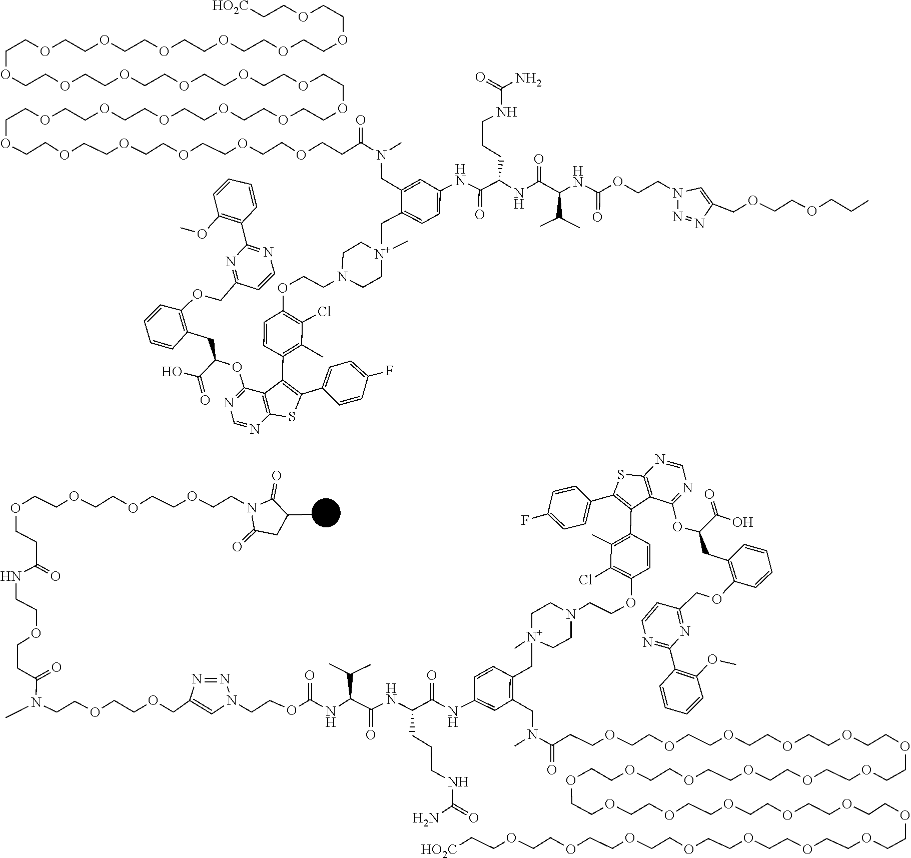 |
| Ab-P1- L21-P1 | 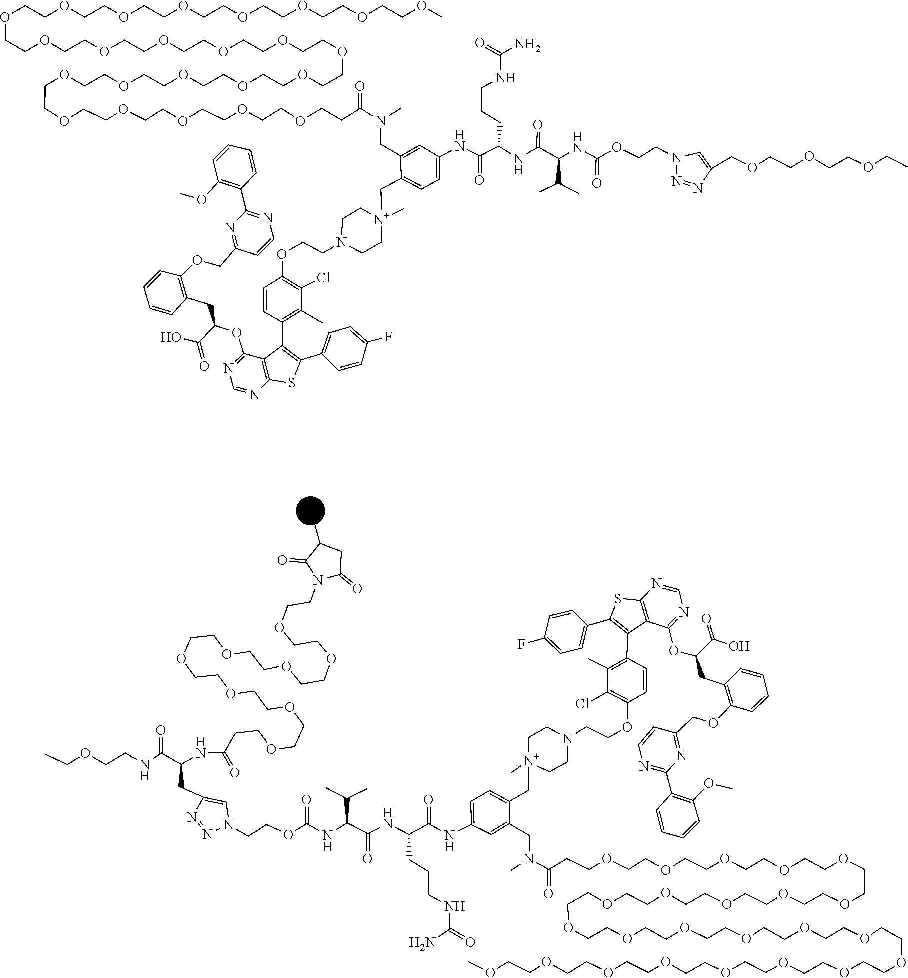 |
| Ab-P1- L22-P1 | 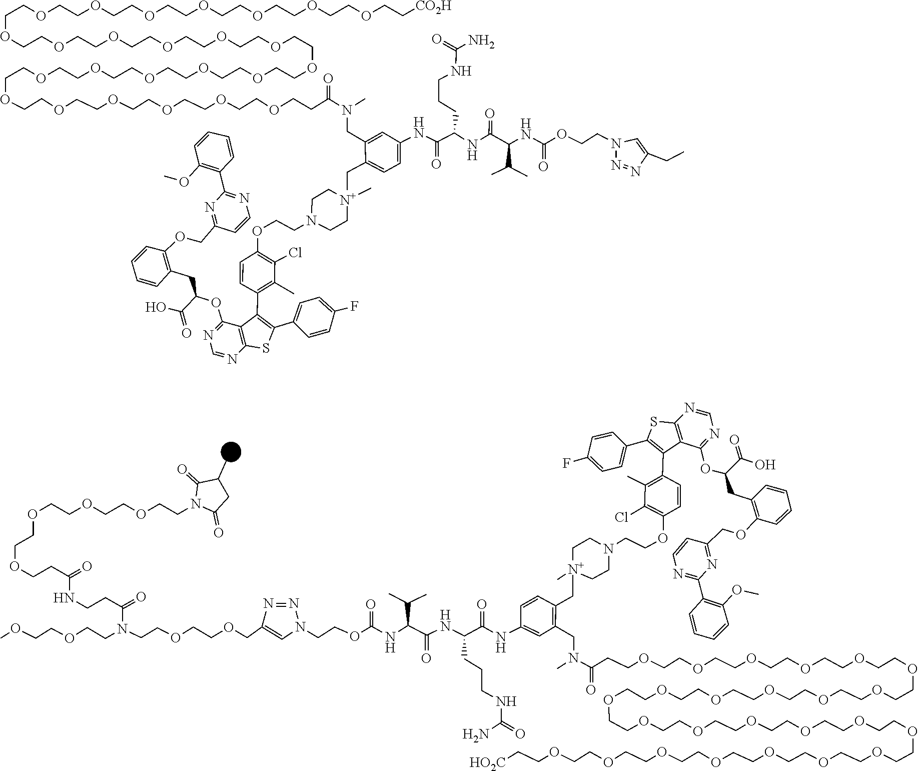 |
| Ab-P1- L23-P1 | 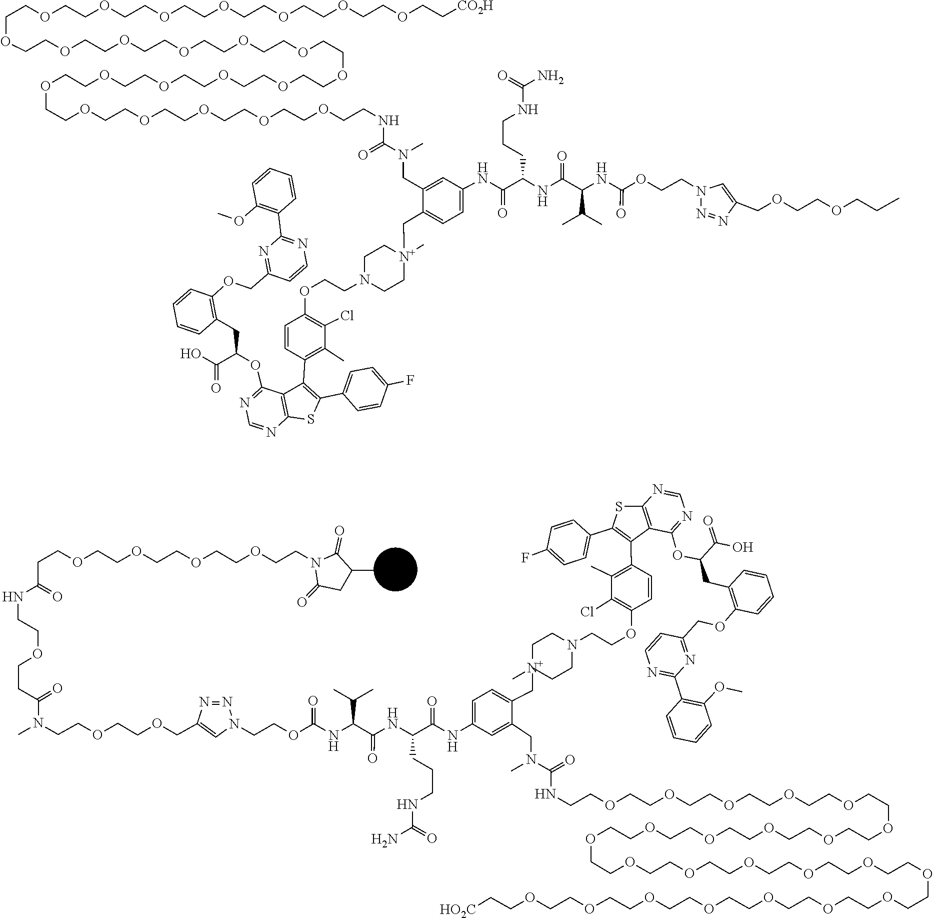 |
| Ab-P1- L24-P1 | 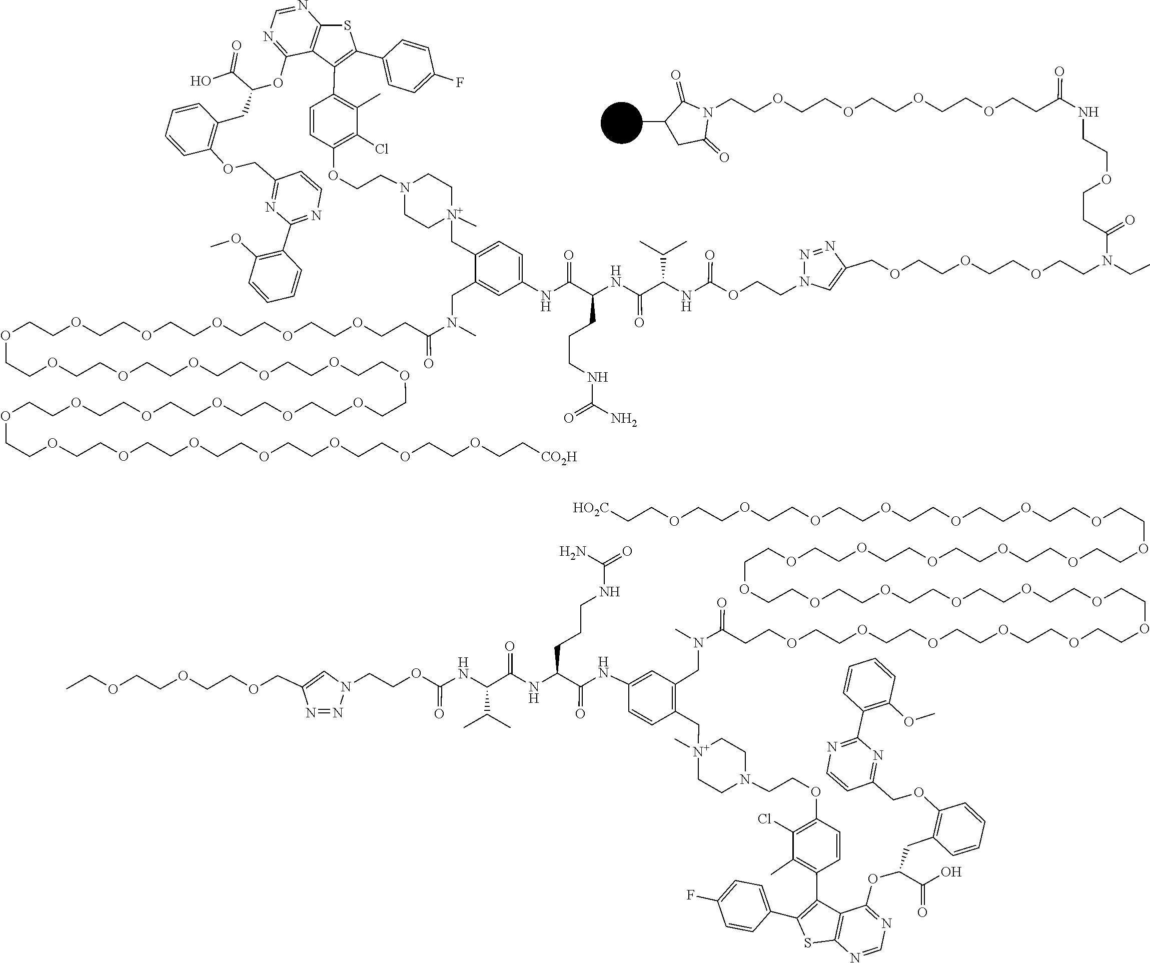 |
| Ab-P1- L25-P1 | 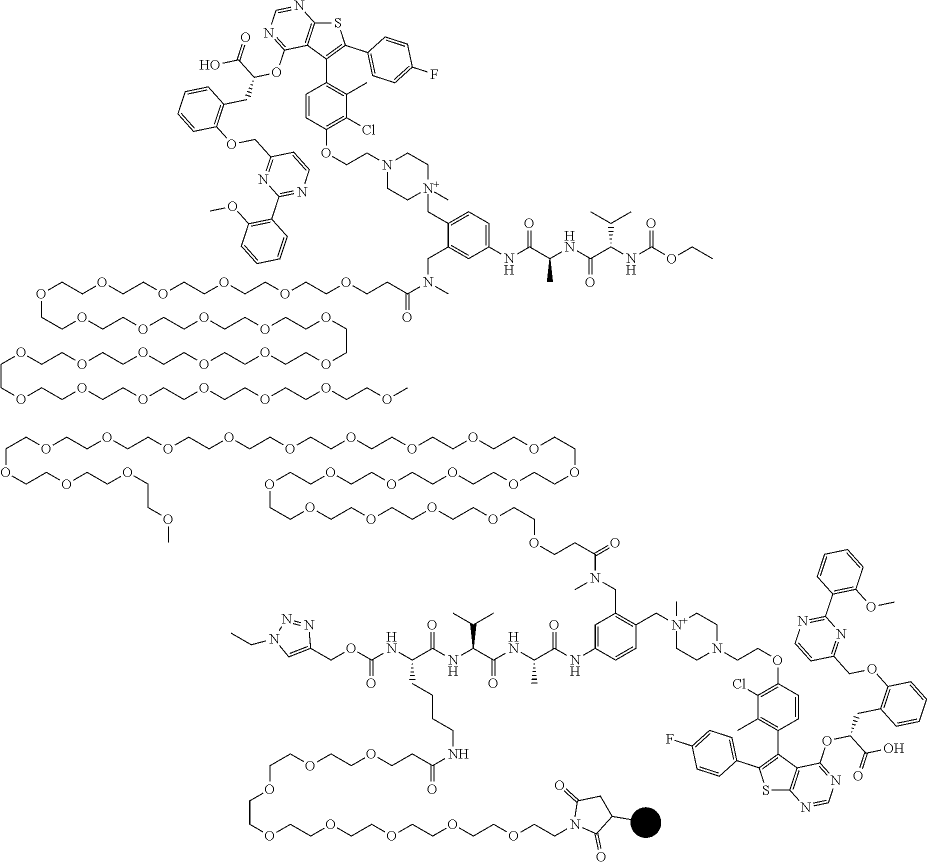 |
| Ab-P1- L26-P1 | 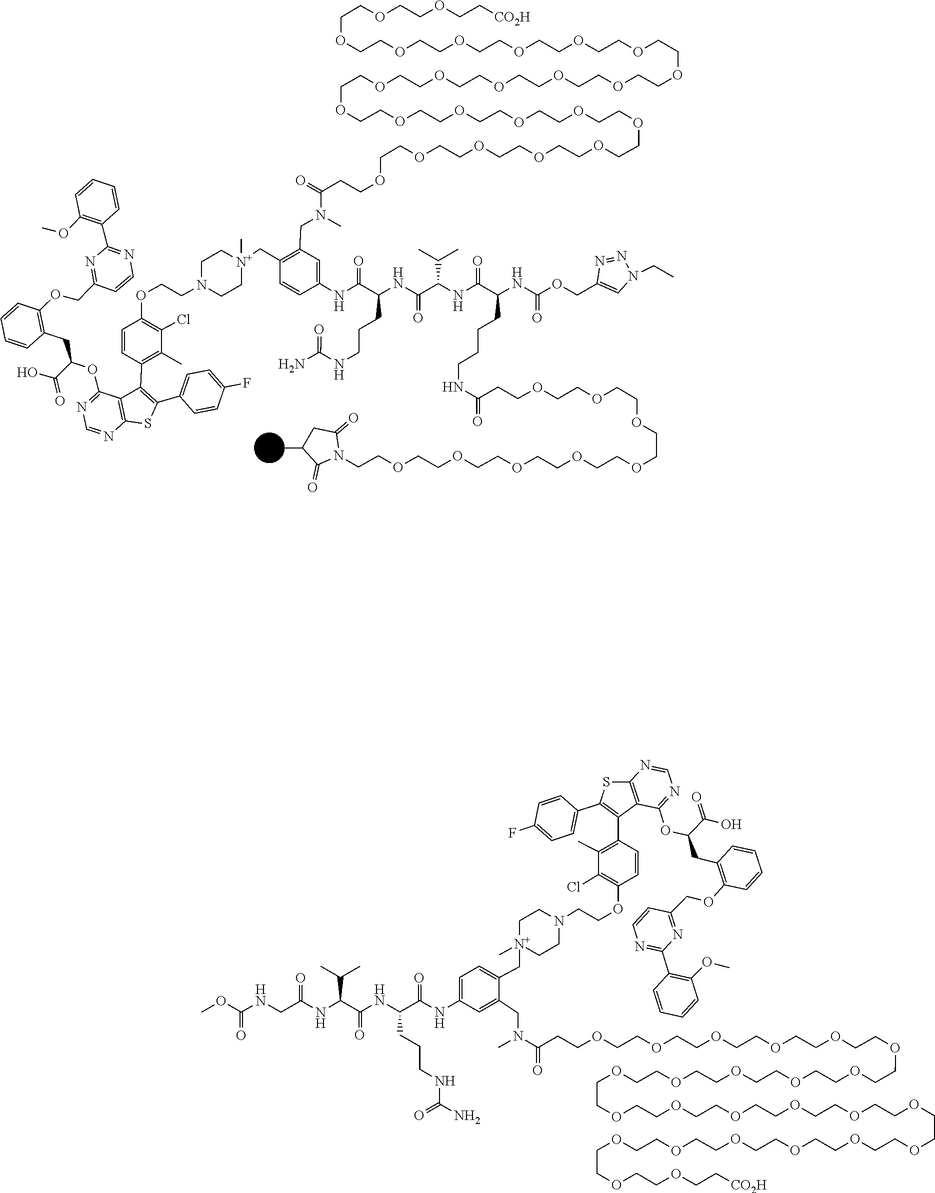 |
| Ab-P1- L27-P1 | 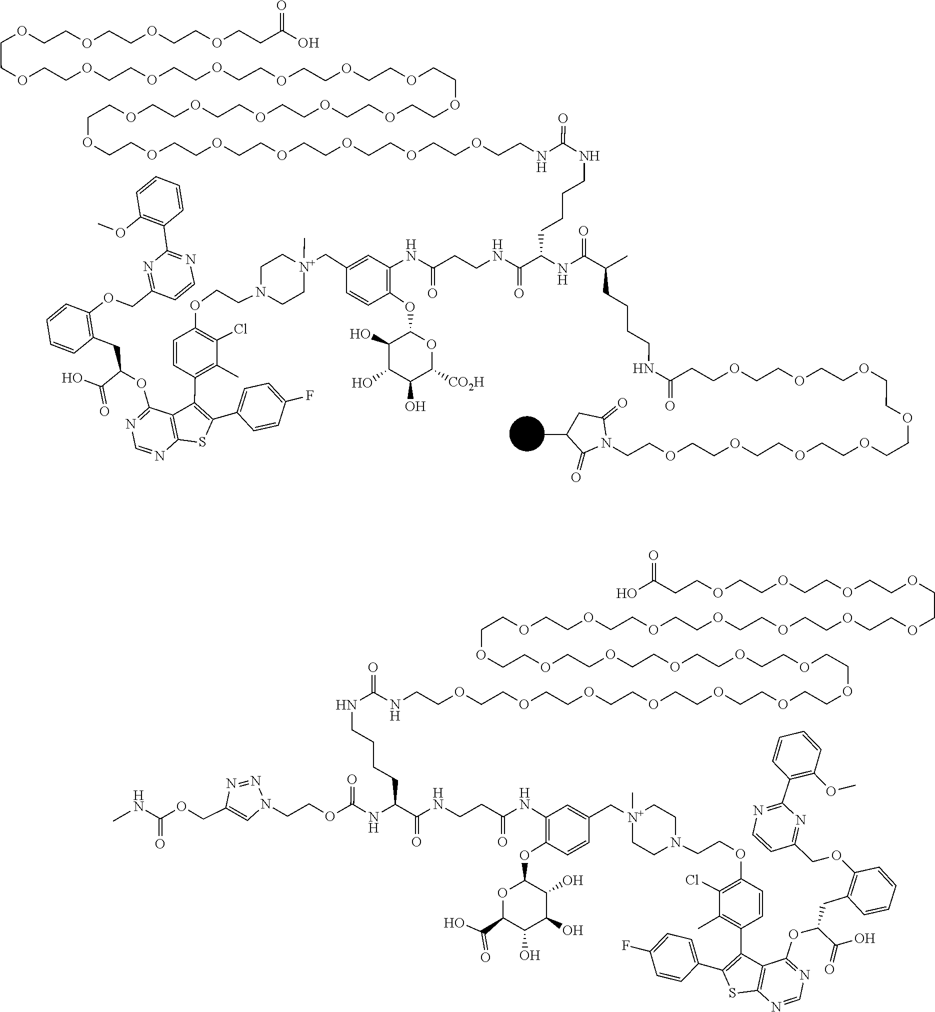 |
| Ab-P1- L28-P1 | 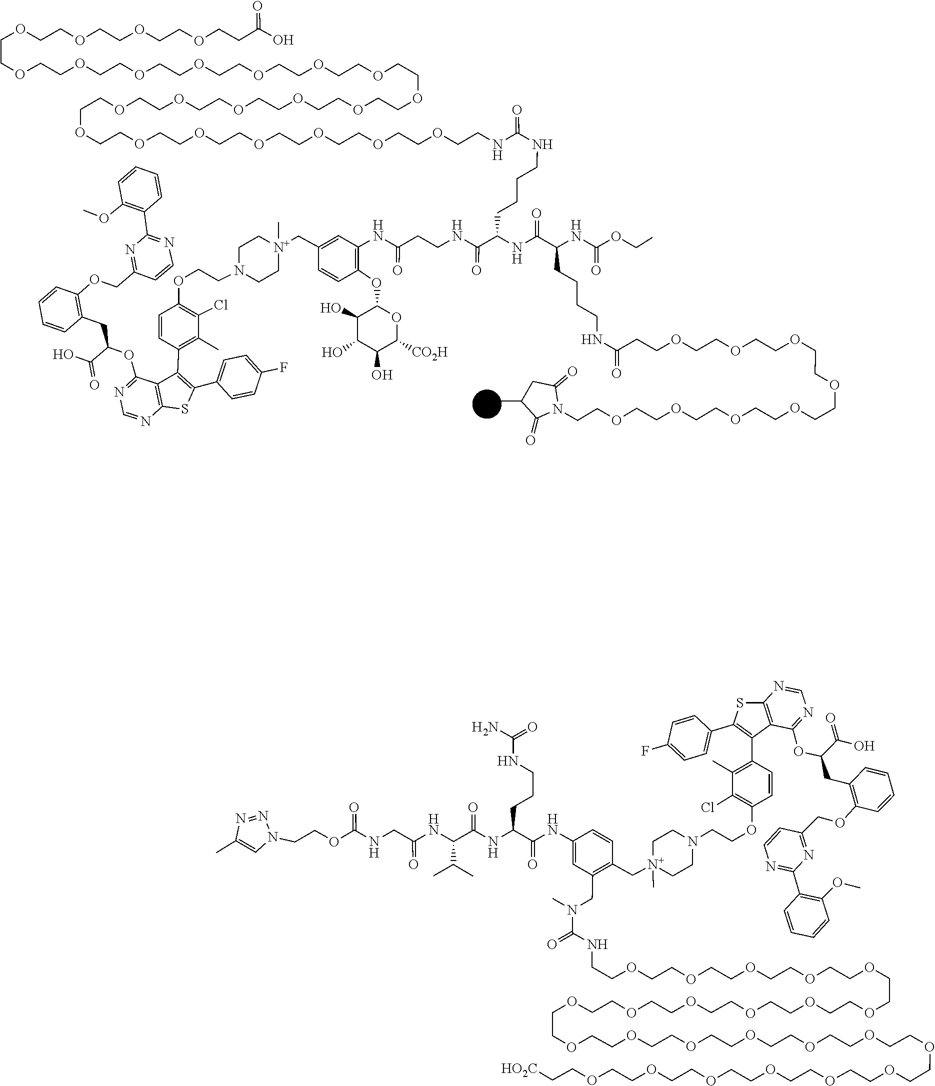 |
| Ab-P1- L29-P2 | 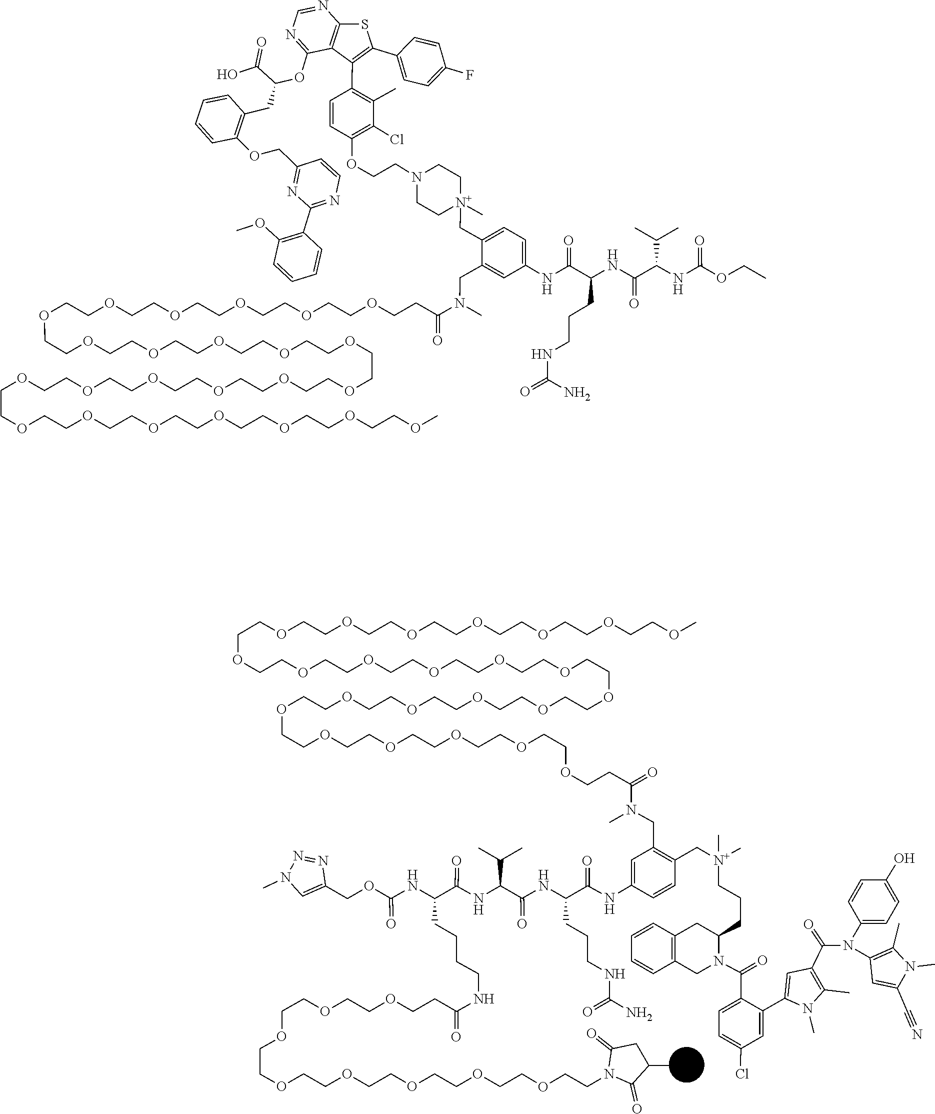 |
| Ab-P2- L29-P1 | 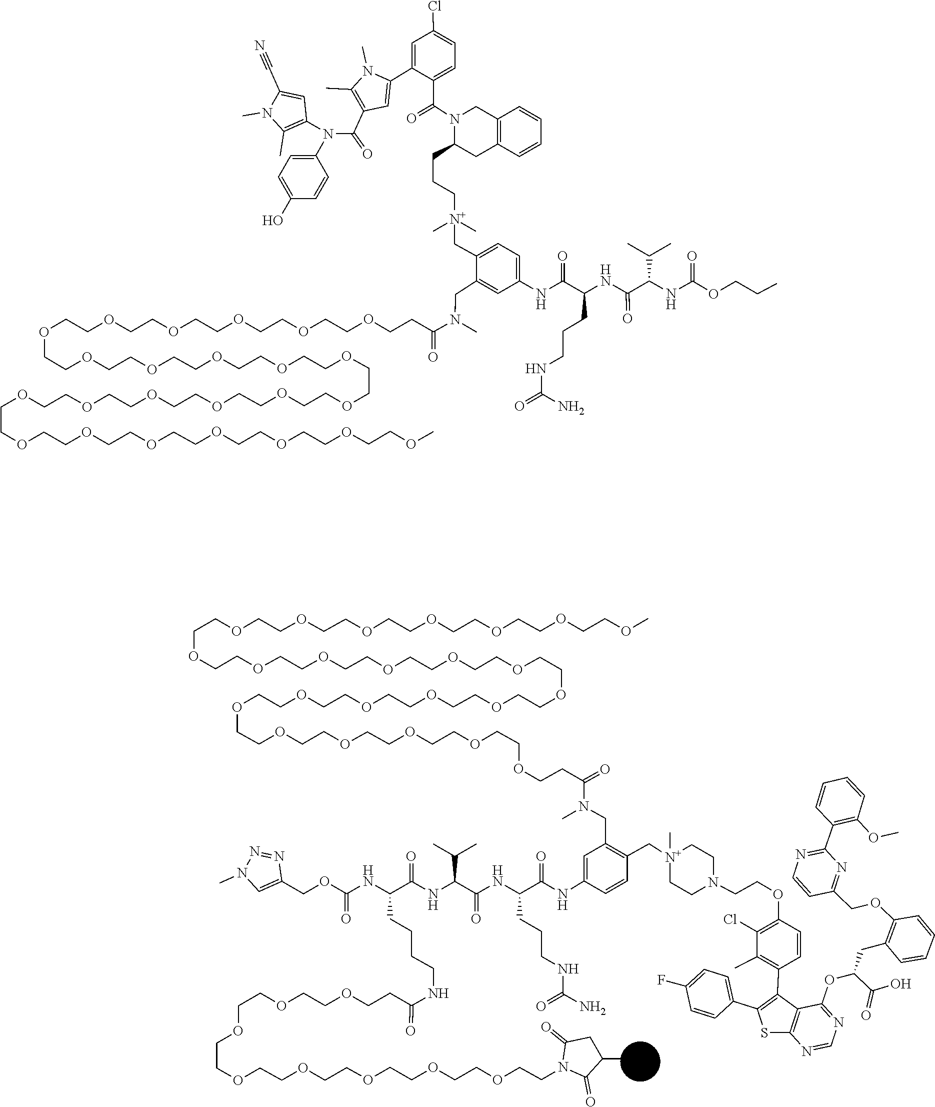 |
| Ab-P1- L30-P2 | 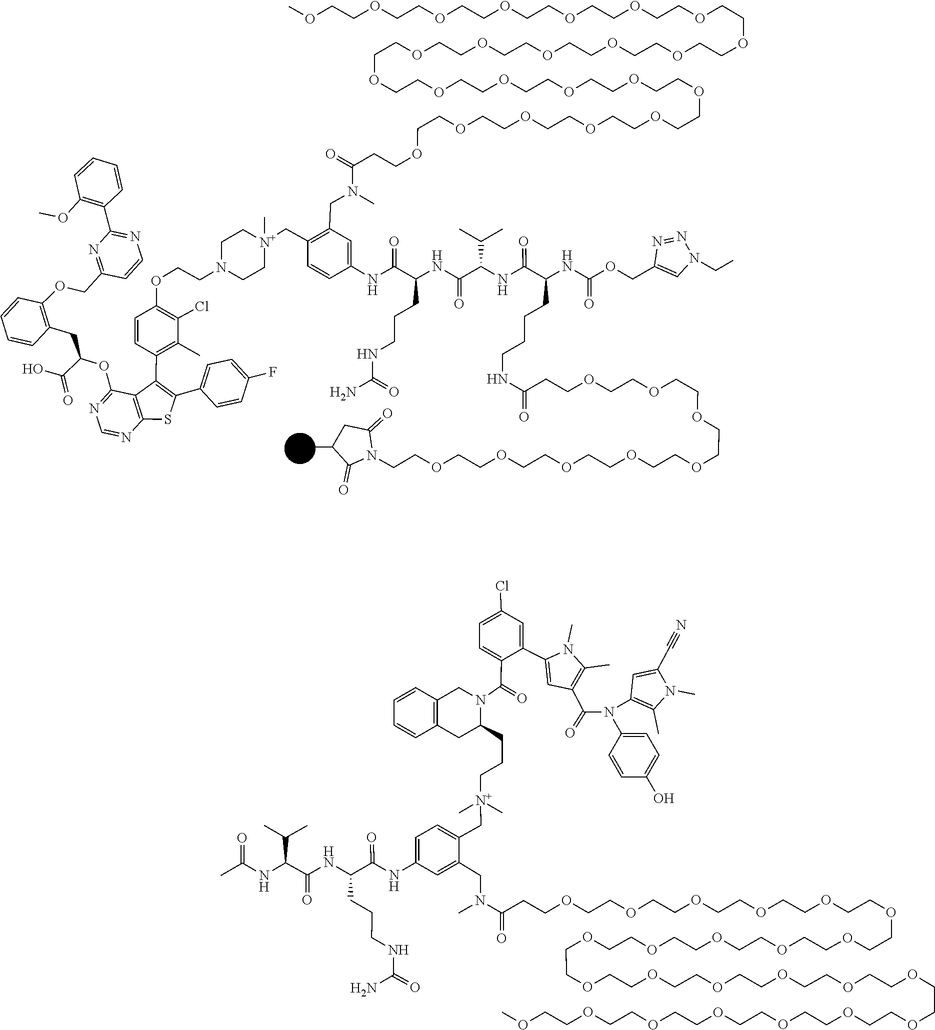 |
| Ab-P1- L31-P2 | 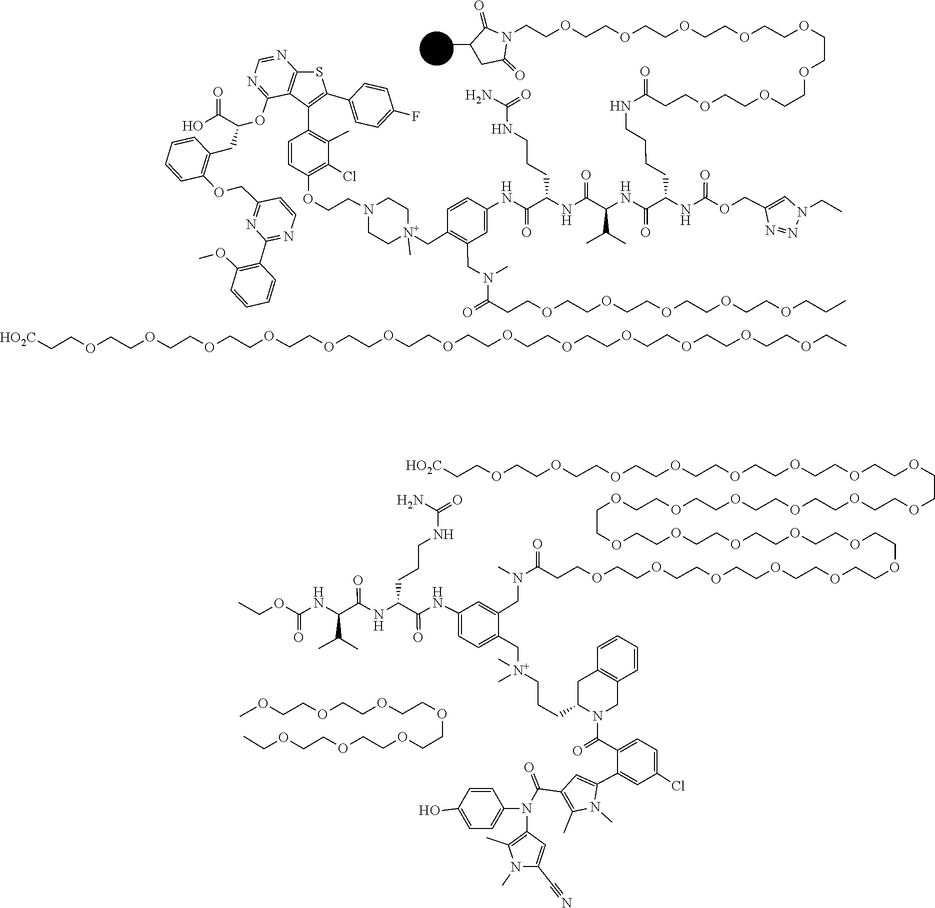 |
| Ab-P1- L32-P2 | 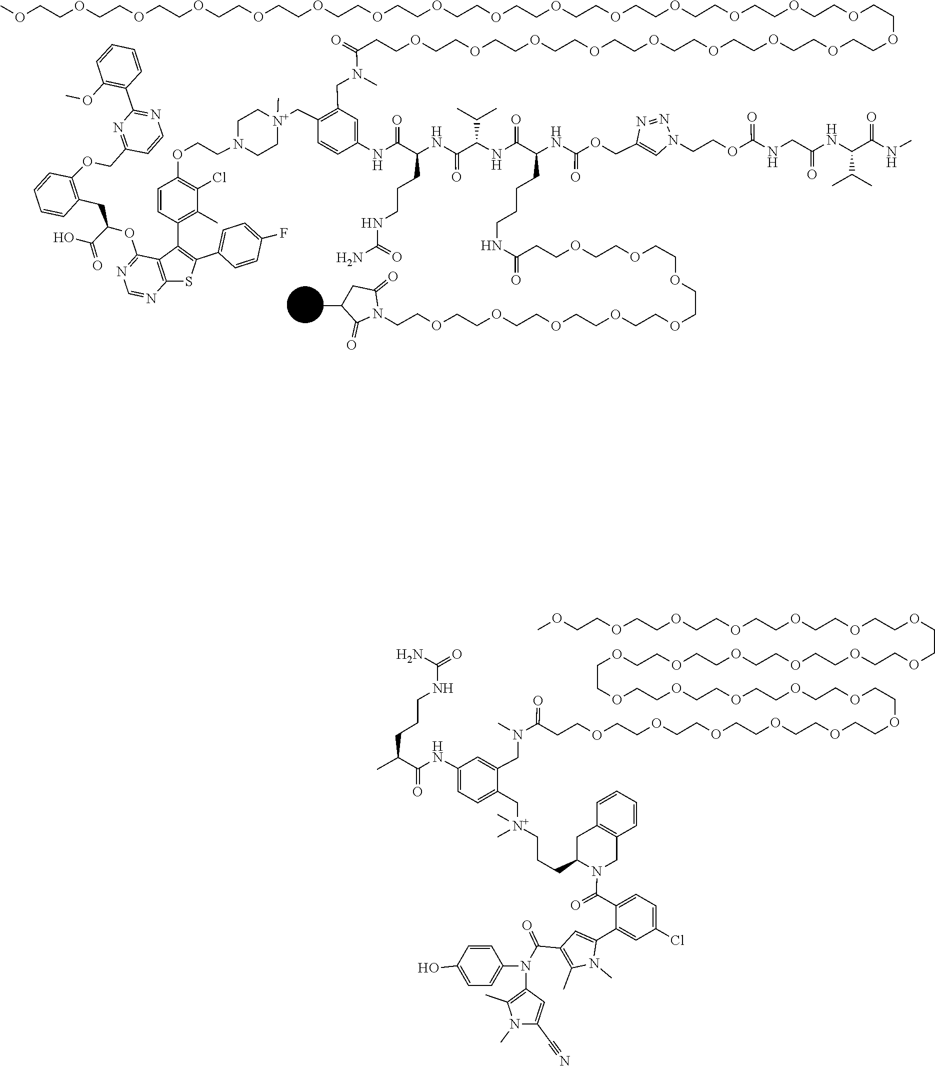 |
| Ab-P1- L33-P1 | 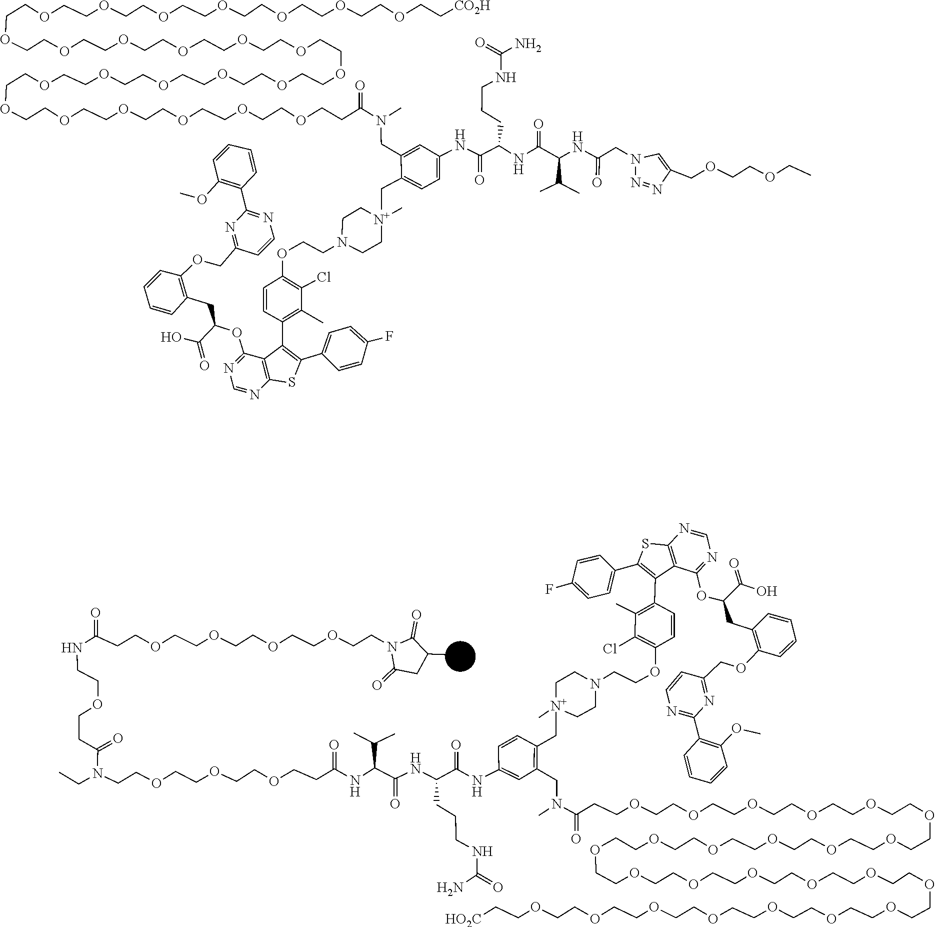 |
| Ab-P1- L34-P1 | 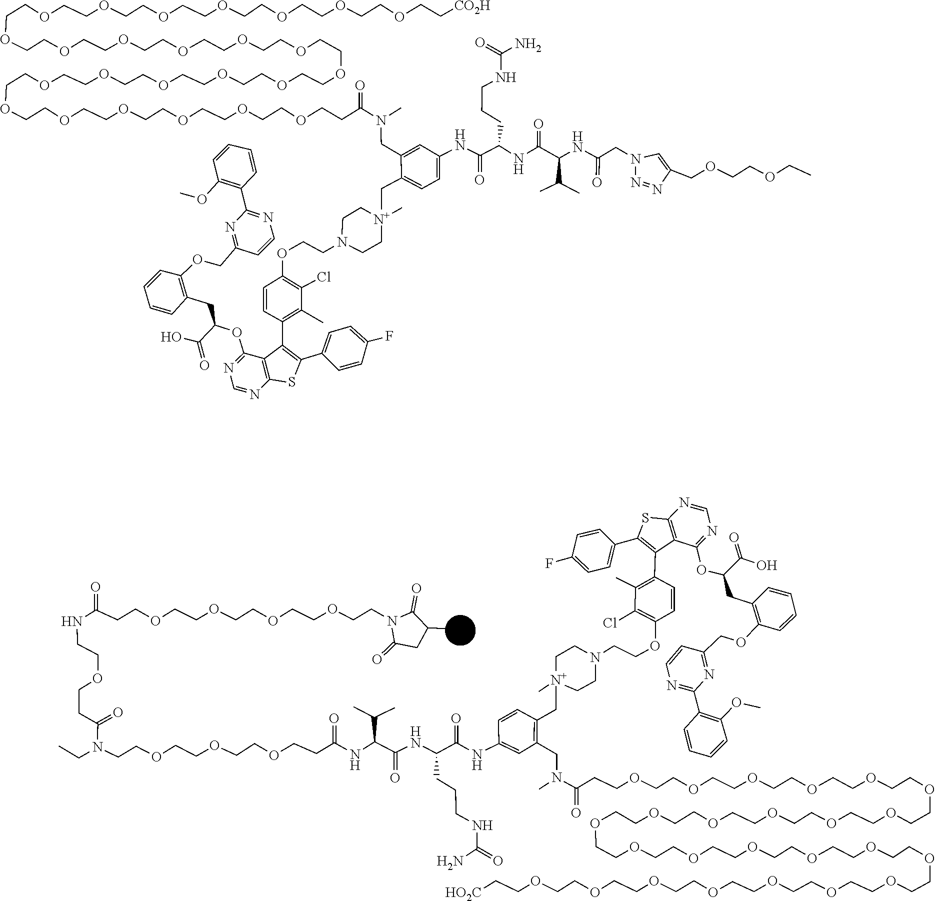 |
| Ab-P1- L35-P1 | 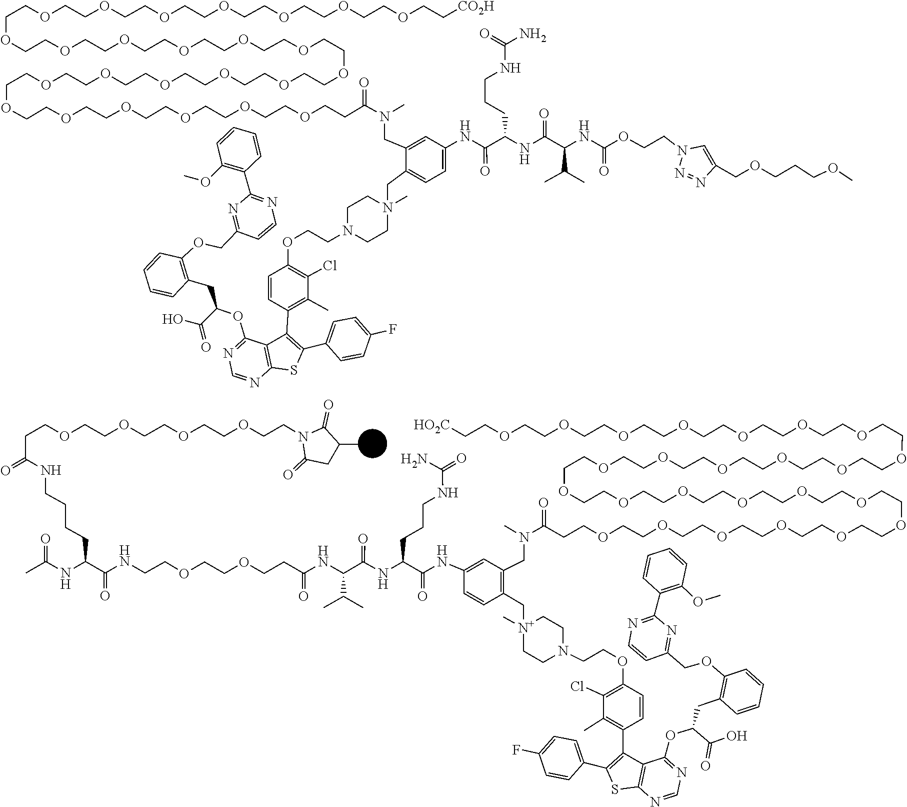 |
| Ab-P1- L36-P1 | 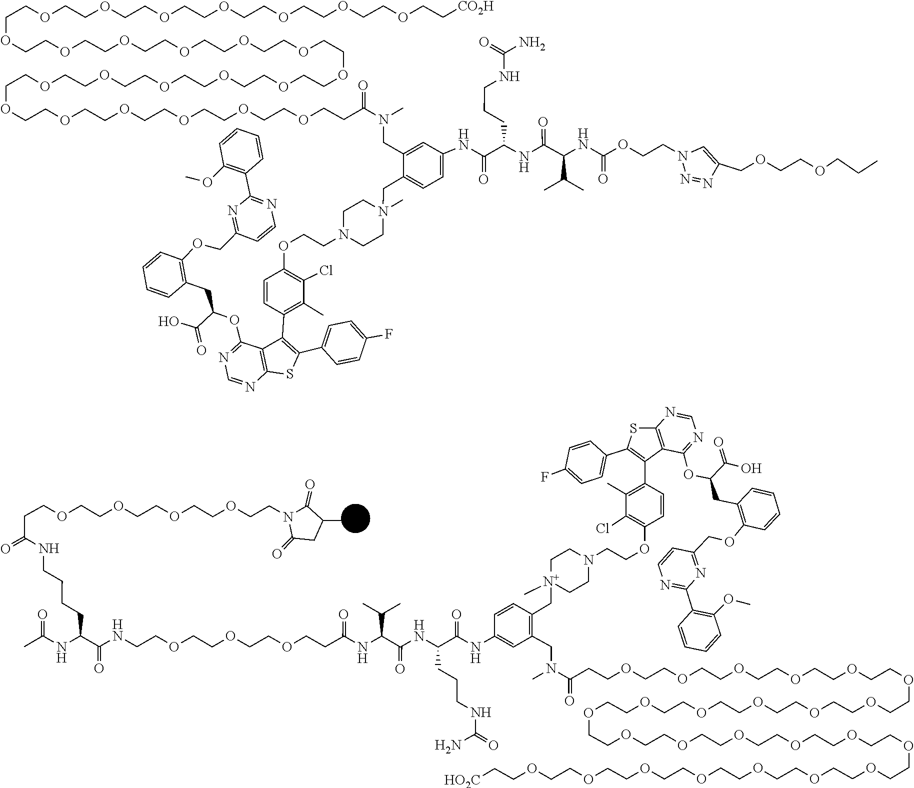 |

The ADCs depicted above can also be represented by the following formula:

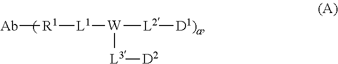

wherein [symbol] represents an antibody or an antigen fragment thereof covalently linked to the linker-payload (L/P) depicted above; a is an integer from 1 to 16. In some embodiments, a is an integer from 1 to 8. In some embodiments, a is an integer from 1 to 5. In some embodiments, a is an integer from 2 to 4. In some embodiments, a is 2. In some embodiments, a is 4. In some embodiments, a is determined by liquid chromatography-mass spectrometry (LC-MS).

In some embodiments, for ADCs depicted in Table C, the antibody is an antibody or an antigen fragment thereof described herein. In some embodiments, the antibody is an anti-HER2 antibody (e.g., trastuzumab, Disitamab or Ab T). In some embodiments, the antibody is an anti-CD74 antibody (e.g., VHmil x VK1aNQ or milatuzumab). In some embodiments, the antibody is an anti-CD48 antibody (e.g., SGN-CD48A (MEM/MEM102) or NY920). In some embodiments, the antibody is an anti-PCAD antibody (e.g., CQY679). In some embodiments, the antibody is an anti-EphA2 antibody (e.g., 1C₁). In some embodiments, the antibody is an anti-MET antibody (e.g., 9006, 9338, or 8902). In some embodiments, the antibody is an anti-TROP2 antibody (e.g., Datopotamab). In some embodiments, the antibody is an anti-B7-H3 antibody (e.g., ABBV-155 or DS-5573a). In some embodiments, the antibody is an anti-5T4 antibody..

As used herein, “P-L-P” refers to the linker-payloads, linker-drugs, or linker-compounds disclosed herein and the terms “P #-L #-P #” refers to a specific dual linker-drug disclosed herein, wherein each of the codes “P #” refers to a specific antineoplastic compound (e.g. BH3 mimetics) unless otherwise specified and L #refers to a specific dual linker unless otherwise specified. The two “P #” codes can be the same or different, i.e. refers to the same or different antineoplastic compounds (e.g. BH3 mimetics). For example, “Pi-L1-P1” refers to the linker-payload compound with dual linker Li attaches to two P1 payloads, while “P1-L1-P2” refers to the linker-payload compound with dual linker L1 attaches to a P1 and a P2 payload, including an enantiomer, diastereoisomer, atropisomer, deuterated derivative, and/or pharmaceutically acceptable salt of any of the foregoing. In some embodiments, for example, when the linker L1 is not symmetrical, the terms “P1-L1-P2” and “P2-L1-P1” refer to two different linker-drugs. In the present disclosure, “L #-P #” refer to a specific mono linker-drug disclosed herein. For example, “L1-P1” refers to the linker-payload compound with mono linker L1 attaches to one P1 payload.

In some embodiments, the antibody or antigen-binding fragment binds to a target antigen on a cancer cell. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74, or EphA2. In some embodiments, the target antigen is MET, CD74, CD48, HER2, TROP2, B7-H3, or 5T4.

In some embodiments, the antibody or antigen-binding fragment are antibodies or antigen-binding fragments disclosed on the internet at go.drugbank.com/drugs/DB00002, in international application publication WO2018/098306, WO2016/179257, WO2011/097627, WO2017/214282, WO2017/214301, WO2017/214233, WO2013/126810, WO2008/056833, WO2020/236817, WO2017/214335, and WO2012147713, and in U.S. Patent No. U.S. Pat. No. 6,870,034B2, which are incorporated by reference in their entireties.

In some embodiments, the antibody or antigen-binding fragment is an anti-EphA2 antibody or antigen-binding fragment. In some embodiments, the antibody or antigen-binding fragment is an anti-PCAD antibody or antigen-binding fragment. In some embodiments, the antibody or antigen-binding fragment is an anti-HER2 antibody or antigen-binding fragment. In some embodiments, the antibody or antigen-binding fragment is an anti-CD48 antibody or antigen-binding fragment. In some embodiments, the antibody or antigen-binding fragment is an anti-CD74 antibody or antigen-binding fragment. In some embodiments, the present disclosure provides an antibody or antigen-binding fragment comprising one or more SEQ IDs listed in Tables D2-D5, D7 and D8 described herein.

[141]Also provided herein, in some embodiments, are compositions comprising multiple copies of an antibody-drug conjugate (e.g., any of the exemplary antibody-drug conjugates described herein). In some embodiments, the average p of the antibody-drug conjugates in the composition is from about 2 to about 4.

Also provided herein, in some embodiments, are pharmaceutical compositions comprising an antibody-drug conjugate (e.g., any of the exemplary antibody-drug conjugates described herein) or a composition (e.g., any of the exemplary compositions described herein), and a pharmaceutically acceptable carrier.

Further provided herein, in some embodiments, are therapeutic uses for the described ADC compounds and compositions, e.g., in treating a cancer. In some embodiments, the present disclosure provides methods of treating a cancer (e.g., a cancer that expresses an antigen targeted by the antibody or antigen-binding fragment of the ADC, such as PCAD, HER2, CD48, CD74, EphA2, MET, TROP2, B7-H3, or 5T4). In some embodiments, the present disclosure provides methods of reducing or slowing the expansion of a cancer cell population in a subject. In some embodiments, the present disclosure provides methods of determining whether a subject having or suspected of having a cancer will be responsive to treatment with an ADC compound or composition disclosed herein.

An exemplary embodiment is a method of treating a subject having or suspected of having a cancer, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74 or EphA2. In some embodiments, the target antigen is CD74, CD48, HER2, TROP2, B7-H3, or 5T4. In some embodiments, the target antigen is MET. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

[145]Another exemplary embodiment is a method of reducing or inhibiting the growth of a tumor in a subject, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the tumor expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74 or EphA2. In some embodiments, the target antigen is CD74, CD48, HER2, TROP2, B7-H3, or 5T4. In some embodiments, the target antigen is MET. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, pancreatic cancer, stomach cancer, colon cancer, head and neck cancer, or spleen cancer. In some embodiments, the tumor is a gastric cancer. In some embodiments, administration of the antibody-drug conjugate, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%.

[146]Another exemplary embodiment is a method of reducing or slowing the expansion of a cancer cell population in a subject, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the cancer cell population expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74 or EphA2. In some embodiments, the target antigen is CD74, CD48, HER2, TROP2, B7-H3, or 5T4. In some embodiments, the target antigen is MET. In some embodiments, the cancer cell population is from a tumor or a hematological cancer. In some embodiments, the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer cell population is from a lymphoma or gastric cancer. In some embodiments, administration of the antibody-drug conjugate, composition, or pharmaceutical composition reduces the cancer cell population by at least about 10%, at least about 20%, at least about 30%, at least about 40%, at least about 50%, at least about 60%, at least about 70%, at least about 80%, at least about 90%, at least about 95%, or at least about 99%. In some embodiments, administration of the antibody-drug conjugate, composition, or pharmaceutical composition slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%.

[147]Another exemplary embodiment is an antibody-drug conjugate, composition, or pharmaceutical composition (e.g., any of the exemplary antibody-drug conjugates, compositions, or pharmaceutical compositions disclosed herein) for use in treating a subject having or suspected of having a cancer. In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-Rᵢ, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74 or EphA2. In some embodiments, the target antigen is CD74, CD48, HER2, TROP2, B7-H3, or 5T4. In some embodiments, the target antigen is MET. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a use of an antibody-drug conjugate, composition, or pharmaceutical composition (e.g., any of the exemplary antibody-drug conjugates, compositions, or pharmaceutical compositions disclosed herein) in treating a subject having or suspected of having a cancer. In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74 or EphA2. In some embodiments, the target antigen is CD74, CD48, HER2, TROP2, B7-H3, or 5T4. In some embodiments, the target antigen is MET. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a use of an antibody-drug conjugate, composition, or pharmaceutical composition (e.g., any of the exemplary antibody-drug conjugates, compositions, or pharmaceutical compositions disclosed herein) in a method of manufacturing a medicament for treating a subject having or suspected of having a cancer. In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74 or EphA2. In some embodiments, the target antigen is CD74, CD48, HER2, TROP2, B7-H3, or 5T4. In some embodiments, the target antigen is MET. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a method of determining whether a subject having or suspected of having a cancer will be responsive to treatment with an antibody-drug conjugate, composition, or pharmaceutical composition (e.g., any of the exemplary antibody-drug conjugates, compositions, or pharmaceutical compositions disclosed herein) by providing a biological sample from the subject; contacting the sample with the antibody-drug conjugate; and detecting binding of the antibody-drug conjugate to cancer cells in the sample. In some embodiments, the cancer cells in the sample express a target antigen. In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74 or EphA2. In some embodiments, the target antigen is CD74, CD48, HER2, TROP2, B7-H3, or 5T4. In some embodiments, the target antigen is MET. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer. In some embodiments, the sample is a tissue biopsy sample, a blood sample, or a bone marrow sample.

Methods of producing the described ADC compounds and compositions are also disclosed. An exemplary embodiment is a method of producing an antibody-drug conjugate by reacting an antibody or antigen-binding fragment with a dual linker joined or covalently attached to two antineoplastic compounds, wherein at least one antineoplastic compound is a BH3 mimetic (e.g., two BH3 mimetics or a BH3 mimetic and a non-BH3 mimetic (e.g., topoisomerase I inhibitor)) under conditions that allow conjugation.

### BRIEF DESCRIPTION OF THE DRAWINGS

FIG. 1 shows Tumor volume (mm³) of H929-grafted female SCID mice upon treatment with IgGI-CysmAb Fc silent_P1-L19-P2, anti-CD48 MEM_CysmAb Fc silent, anti-CD48 MEM_CysmAb Fc silent_P1-L19-P2 at 30 mg/kg, administered once IV (n=8).

FIG. 2 shows % of body weight loss of H929-grafted female SCID mice upon treatment with IgGI-CysmAb Fc silent_P1-L19-P2, anti-CD48 MEM_CysmAb Fc silent, anti-CD48 MEM_CysmAb Fc silent_P1-L19-P2 at 30 mg/kg, administered once IV (n=8).

FIG. 3 shows tumor volume (mm³) of H929-grafted female SCID mice upon treatment with IgGI-CysmAb Fc WT_P1-L19-P2, anti-CD48 MEM_CysmAb Fc WT_P1-L19-P2, anti-CD48 MEM_CysmAb Fc WT_P1-L29-P2, anti-CD48 MEM_CysmAb Fc WT_P2-L29-P1, anti-CD48 MEM_CysmAb Fc WT_P1-L31-P2, anti-CD48 MEM_CysmAb Fc WT_P1-L32-P2 and anti-CD48 MEM_CysmAb Fc WT_P1-L30-P2 administrated at 30 mg/kg once IV, (n=6)

FIG. 4 shows body weight of H929-grafted female SCID mice upon treatment with IgG1-CysmAb Fc WT_P1-L19-P2, anti-CD48 MEM_CysmAb Fc WT_P1-L19-P2, anti-CD48 MEM_CysmAb Fc WT_P1-L29-P2, anti-CD48 MEM_CysmAb Fc WT_P2-L29-P1, anti-CD48 MEM_CysmAb Fc WT_P1-L31-P2, anti-CD48 MEM_CysmAb Fc WT_P1-L32-P2 and anti-CD48 MEM_CysmAb Fc WT_P1-L30-P2 administrated at 30 mg/kg once IV, (n=6).

FIG. 5 shows tumor volume (mm³) of KMS-21-BM-grafted female NSG mice upon treatment with IgGI-CysmAb Fc silent_P1-L29-P2, anti-CD48 MEM_CysmAb Fc silent, anti-CD48 MEM_CysmAb Fc silent_P1-L29-P2 administered once IV (n=6).

FIG. 6 shows body weight of KMS-21-BM-grafted female NSG mice upon treatment with IgG1-CysmAb Fc silent_P1-L29-P2, anti-CD48 MEM_CysmAb Fc silent, anti-CD48 MEM_CysmAb Fc silent_P1-L29-P2 administered once IV (n=6).

FIG. 7 shows tumor volume (mm³) of KMS27-grafted female NSG mice upon treatment with IgGI-CysmAb Fc silent_P1-L19-P2, anti-CD48 MEM102_CysmAb Fc silent and anti-CD48 MEM102_CysmAb Fc silent_P1-L19-P2 at 10 and/or 30 mg/kg, once IV (n=6).

FIG. 8 shows body weight of KMS27-grafted female NSG mice upon treatment with IgG1-CysmAb Fc silent_P1-L19-P2, anti-CD48 MEM102_CysmAb Fc silent and anti-CD48 MEM102_CysmAb Fc silent_P1-L19-P2 at 10 and/or 30 mg/kg, once IV (n=6).

FIG. 9 is a graph showing dose response curves of three ADCs - Datopotamab-P5-L12-P7, Datopotamab-mono-L1-P5, and Datopotamab-mono-L3-P8 in the NCI-H441 cell line.

FIG. 10A are graphs showing dose response curves of five ADCs - Trastuzumab-mono-L3-P8, Trastuzumab-mono-L3-P8, Trastuzumab-mono-L1-P5, Trastuzumab-mono-L1-P7, and Trastuzumab-P5-L12-P7 in the HCC1419 and ZR-75-30 cell lines.

FIG. 10B is a graph showing dose response curves of four ADCs - Trastuzumab-mono-L3-P8, Disitmab-mono-L1-P5, Disitmab-mono-L3-P8, and Disitmab-P5-L12-P7 in the UACC-812 cell line.

FIG. 11 is a graph showing dose response curves of three ADCs - NY920-P5-L12-P4, NY920-mono-L1-P4, and NY920-mono-L2-P5 in the KMS-27 cell line.

FIG. 12 is a graph showing dose response curves of three ADCs - VHmil x VKlaNQ-P5-L12-P4, VHmil x VKlaNQ-mono-L1-P4, and VHmil x VKlaNQ-mono-L2-P5 in the EOL-1 cell line.

### DETAILED DESCRIPTION OF ILLUSTRATIVE EMBODIMENTS

The disclosed compositions and methods may be understood more readily by reference to the following detailed description taken in connection with the accompanying figures, which form a part of this disclosure.

Throughout this text, the descriptions refer to compositions and methods of using the compositions. Where the disclosure describes or claims a feature or embodiment associated with a composition, such a feature or embodiment is equally applicable to the methods of using the composition. Likewise, where the disclosure describes or claims a feature or embodiment associated with a method of using a composition, such a feature or embodiment is equally applicable to the composition.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise. All references cited herein are incorporated by reference for any purpose. Where a reference and the specification conflict, the specification will control.

Unless the context of a description indicates otherwise, e.g., in the absence of symbols indicating specific point(s) of connectivity, when a structure or fragment of a structure is drawn, it may be used on its own or attached to other components of an ADC, and it may do so with any orientation, e.g., with the antibody attached at any suitable attachment point to a chemical moiety such as a linker-drug. Where indicated, however, components of an ADC are attached in the orientation shown in a given formula. For example, if Formula (1) is described as

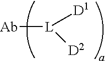

and the group

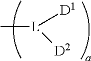

is described as

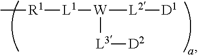

then the elaborated structure of Formula (1) is

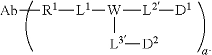

It is neither

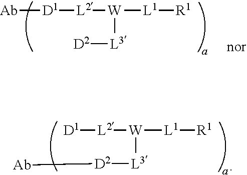

It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

As used throughout this application, antibody drug conjugates can be identified using a naming convention in the general format of “target antigen/antibody-payload-dual linker-payload”. For example only, if an antibody drug conjugate is referred to as “Target X-P1-L1-P2”, such a conjugate would comprise an antibody that binds Target X, a dual linker designated as L1, and two payloads designated as P1 and P2, respectively. Alternatively, if an antibody drug conjugate is referred to as “anti-Target X-P1-L1-P2”, such a conjugate would comprise an antibody that binds Target X, a dual linker designated as LI, and two payloads designated as P1 and P2, respectively. In another alternative, if an antibody drug conjugate is referred to as “AbX-P1-L1-P2”, such a conjugate would comprise the antibody designated as AbX, a dual linker designated as L1, and two payloads designated as P1 and P2, respectively. A control antibody drug conjugate comprising a non-specific, isotype control antibody may be referenced as “isotype control IgGi-P1-L1-P2” or “IgGI-P1-L1-P2”.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen, carbon, nitrogen, oxygen, fluorine, and chlorine, such as ³H, ¹¹C, ¹³C, ¹⁴C, ¹⁵N, ¹⁸F, and ³⁶Cl. Accordingly, it should be understood that the present disclosure includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies. Isotopically-labeled compounds can generally be prepared by conventional techniques known to those skilled in the art, e.g., using an appropriate isotopically-labeled reagents in place of the non-labeled reagent previously employed.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise. The terms “comprising”, “having”, “being of” as in “being of a chemical formula”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

The term “about” or “approximately,” when used in the context of numerical values and ranges, refers to values or ranges that approximate or are close to the recited values or ranges such that the embodiment may perform as intended, as is apparent to the skilled person from the teachings contained herein. In some embodiments, about means plus or minus 20%, 15%, 10%, 5%, 1%, 0.5%, or 0.1% of a numerical amount. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

The terms “antibody-drug conjugate,” “antibody conjugate,” “conjugate,” “immunoconjugate,” and “ADC” are used interchangeably, and refer to one or more therapeutic compounds (e.g., an antineoplastic payload, such as a BH3 mimetic moiety, a topoisomerase 1 inhibitor, or an anti-mitotic drug) that is linked to one or more antibodies or antigen-binding fragments. In some embodiments, the

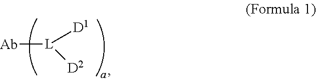

ADC is defined by the generic formula: D2 a (Formula 1), wherein Ab=an antibody or antigen-binding fragment, L=a dual linker moiety, D¹ and D²=a drug moiety (e.g., a Mcl-1 inhibitor, Bcl-2 inhibitor, Bcl-xL inhibitor drug moiety), and a=the number of dual linker moieties with attached D¹ and D² per antibody or antigen-binding fragment. In ADCs comprising antineoplastic payloads (e.g. BH3 mimetic moieties, topoisomerase 1 inhibitors, or anti-mitotic drugs), “2a” refers to the number of antineoplastic payloads (e.g. BH3 mimetic compounds, topoisomerase 1 inhibitors, or anti-mitotic drugs) linked to the antibody or antigen-binding fragment.

The term “antibody” is used in the broadest sense to refer to an immunoglobulin molecule that recognizes and specifically binds to a target, such as a protein, polypeptide, carbohydrate, polynucleotide, lipid, or combinations of the foregoing through at least one antigen recognition site within the variable region of the immunoglobulin molecule. An antibody can be polyclonal or monoclonal, multiple or single chain, or an intact immunoglobulin, and may be derived from natural sources or from recombinant sources. An “intact” antibody is a glycoprotein that typically comprises at least two heavy (H) chains and two light (L) chains inter-connected by disulfide bonds. Each heavy chain is comprised of a heavy chain variable region (abbreviated herein as VH) and a heavy chain constant region. The heavy chain constant region comprises three domains, CHI, CH₂ and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FRI, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgGI, IgG2, IgG3, IgG4, IgA1 and IgA2), or subclass. An antibody can be an intact antibody or an antigen-binding fragment thereof.

In some embodiments, the antibody or antibody fragment disclosed herein include modified or engineered amino acid residues, e.g., one or more cysteine residues, as sites for conjugation to a drug moiety (Junutula J R, et al., Nat Biotechnol 2008, 26:925-932). In one embodiment, the disclosure provides a modified antibody or antibody fragment comprising a substitution of one or more amino acids with cysteine at the positions described herein. Sites for cysteine substitution are in the constant regions of the antibody or antibody fragment and are thus applicable to a variety of antibody or antibody fragment, and the sites are selected to provide stable and homogeneous conjugates. A modified antibody or fragment can have one, two or more cysteine substitutions, and these substitutions can be used in combination with other modification and conjugation methods as described herein. Methods for inserting cysteine at specific locations of an antibody are known in the art, see, e.g., Lyons et al., (1990) Protein Eng., 3:703-708, WO 2011/005481, WO2014/124316, WO 2015/138615. In certain embodiments, a modified antibody comprises a substitution of one or more amino acids with cysteine on its constant region selected from positions 117, 119, 121, 124, 139, 152, 153, 155, 157, 164, 169, 171, 174, 189, 191, 195, 197, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody, and wherein the positions are numbered according to the EU system. In some embodiments a modified antibody or antibody fragment comprises a substitution of one or more amino acids with cysteine on its constant region selected from positions 107, 108, 109, 114, 129, 142, 143, 145, 152, 154, 156, 159, 161, 165, 168, 169, 170, 182, 183, 197, 199, and 203 of a light chain of the antibody or antibody fragment, wherein the positions are numbered according to the EU system, and wherein the light chain is a human kappa light chain. In certain embodiments a modified antibody or antibody fragment thereof comprises a combination of substitution of two or more amino acids with cysteine on its constant regions wherein the combinations comprise substitutions at positions 375 of an antibody heavy chain, position 152 of an antibody heavy chain, position 360 of an antibody heavy chain, or position 107 of an antibody light chain and wherein the positions are numbered according to the EU system. In certain embodiments a modified antibody or antibody fragment thereof comprises a substitution of one amino acid with cysteine on its constant regions wherein the substitution is position 375 of an antibody heavy chain, position 152 of an antibody heavy chain, position 360 of an antibody heavy chain, position 107 of an antibody light chain, position 165 of an antibody light chain or position 159 of an antibody light chain and wherein the positions are numbered according to the EU system, and wherein the light chain is a kappa chain. In particular embodiments a modified antibody or antibody fragment thereof comprises a combination of substitution of two amino acids with cysteine on its constant regions wherein the combinations comprise substitutions at positions 375 of an antibody heavy chain and position 152 of an antibody heavy chain, wherein the positions are numbered according to the EU system. In particular embodiments a modified antibody or antibody fragment thereof comprises a substitution of one amino acid with cysteine at position 360 of an antibody heavy chain, wherein the positions are numbered according to the EU system. In other particular embodiments a modified antibody or antibody fragment thereof comprises a substitution of one amino acid with cysteine at position 107 of an antibody light chain and wherein the positions are numbered according to the EU system, and wherein the light chain is a kappa chain.

The term “antibody fragment” or “antigen-binding fragment” or “functional antibody fragment,” as used herein, refers to at least one portion of an antibody that retains the ability to specifically interact with (e.g., by binding, steric hinderance, stabilizing/destabilizing, spatial distribution) an epitope of an antigen (e.g., PCAD, HER2, CD48, CD74, EphA2, MET, TROP2, B7-H3, or 5T4). Antigen-binding fragments may also retain the ability to internalize into an antigen-expressing cell. In some embodiments, antigen-binding fragments also retain immune effector activity. The terms antibody, antibody fragment, antigen-binding fragment, and the like, are intended to embrace the use of binding domains from antibodies in the context of larger macromolecules such as ADCs. It has been shown that fragments of a full-length antibody can perform the antigen binding function of a full-length antibody. Examples of antibody fragments include, but are not limited to, Fab, Fab′, F(ab′)₂, Fv fragments, scFv antibody fragments, disulfide-linked Fvs (sdFv), a Fd fragment consisting of the VH and CHi domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, bispecific or multi-specific antibody constructs, ADCs, v-NAR and bis-scFv (see, e.g., Holliger and Hudson (2005) Nat Biotechnol. 23(9):1126-36). Antigen-binding fragments can also be grafted into scaffolds based on polypeptides such as a fibronectin type III (Fn3) (see U.S. Pat. No. 6,703,199, which describes fibronectin polypeptide minibodies). The term “scFv” refers to a fusion protein comprising at least one antigen-binding fragment comprising a variable region of a light chain and at least one antigen-binding fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, an scFv may have the VL and VH variable regions in either order, e.g., with respect to the N-terminal and C-terminal ends of the polypeptide, the scFv may comprise VL-linker-VH or may comprise VH-linker-VL. Antigen-binding fragments are obtained using conventional techniques known to those of skill in the art, and the binding fragments are screened for utility (e.g., binding affinity, internalization) in the same manner as are intact antibodies. Antigen-binding fragments, for example, may be prepared by cleavage of the intact protein, e.g., by protease or chemical cleavage.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991) “Sequences of Proteins of Immunological Interest,” 5th Ed. Public Health Service, National Institutes of Health, Bethesda, MD (“Kabat” numbering scheme); A1-Lazikani et al. (1997) J Mol Biol. 273(4):927-48 (“Chothia” numbering scheme); ImMunoGenTics (IMGT) numbering (Lefranc (2001) Nucleic Acids Res. 29(1):207-9; Lefranc et al. (2003) Dev Comp Immunol. 27(1):55-77) (“IMGT” numbering scheme); or a combination thereof. In a combined Kabat and Chothia numbering scheme for a given CDR region (for example, HC CDR1, HC CDR2, HC CDR3, LC CDR1, LC CDR2, or LC CDR3), in some embodiments, the CDRs correspond to the amino acid residues that are defined as part of the Kabat CDR, together with the amino acid residues that are defined as part of the Chothia CDR. As used herein, the CDRs defined according to the “Chothia” number scheme are also sometimes referred to as “hypervariable loops.”

In some embodiments, under Kabat, the CDR amino acid residues in the heavy chain variable domain (VH) are numbered 31-35 (HCDR1) (e.g., insertion(s) after position 35), 50-65 (HCDR2), and 95-102 (HCDR3); and the CDR amino acid residues in the light chain variable domain (VL) are numbered 24-34 (LCDR1) (e.g., insertion(s) after position 27), 50-56 (LCDR2), and 89-97 (LCDR3). In some embodiments, under Chothia, the CDR amino acids in the VH are numbered 26-32 (HCDR1) (e.g., insertion(s) after position 31), 52-56 (HCDR2), and 95-102 (HCDR3); and the amino acid residues in VL are numbered 26-32 (LCDR1) (e.g., insertion(s) after position 30), 50-52 (LCDR2), and 91-96 (LCDR3). By combining the CDR definitions of both Kabat and Chothia, in some embodiments, the CDRs comprise or consist of, e.g., amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. In some embodiments, under IMGT, the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3). In some embodiments, under IMGT, the CDR regions of an antibody may be determined using the program IMGT/DomainGap Align.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods (see, e.g., U.S. Pat. No. 4,816,567). Monoclonal antibodies may also be isolated from phage antibody libraries using the techniques described in Clackson et al. (1991) Nature 352:624-8, and Marks et al. (1991) J Mol Biol. 222:581-97, for example. The term also includes preparations of antibody molecules of single molecular composition. A monoclonal antibody composition displays a single binding specificity and affinity for a particular epitope.

The monoclonal antibodies described herein can be non-human, human, or humanized. The term specifically includes “chimeric” antibodies, in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity.

The term “human antibody,” as used herein, refers an antibody produced by a human or an antibody having an amino acid sequence of an antibody produced by a human. The term includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik et al. ((2000) J Mol Biol. 296(1):57-86). The structures and locations of immunoglobulin variable domains, e.g., CDRs, may be defined using well known numbering schemes, e.g., the Kabat numbering scheme, the Chothia numbering scheme, or a combination of Kabat and Chothia, and/or ImMunoGenTics (IMGT) numbering. The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody,” as used herein, is not intended to include antibodies in which CDR sequences derived from the germline of another mammalian species, such as a mouse, have been grafted onto human framework sequences.

The term “recombinant human antibody,” as used herein, refers to a human antibody that is prepared, expressed, created, or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody,” as used herein, refers to antibodies wherein the amino acid sequence of the immunoglobulin molecule is derived from two or more species. In some instances, the variable regions of both heavy and light chains correspond to the variable regions of antibodies derived from one species with the desired specificity, affinity, and activity while the constant regions are homologous to antibodies derived from another species (e.g., human) to minimize an immune response in the latter species.

As used herein, the term “humanized antibody” refers to forms of antibodies that contain sequences from non-human (e.g., murine) antibodies as well as human antibodies. Such antibodies are a type of chimeric antibody which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin. The humanized antibody can be further modified by the substitution of residues, either in the Fv framework region and/or within the replaced non-human residues to refine and optimize antibody specificity, affinity, and/or activity.

The term “Fc region,” as used herein, refers to a polypeptide comprising the CH3, CH₂ and at least a portion of the hinge region of a constant domain of an antibody. Optionally, an Fc region may include a CH4 domain, present in some antibody classes. An Fc region may comprise the entire hinge region of a constant domain of an antibody. In some embodiments, an antibody or antigen-binding fragment comprises an Fc region and a CHi region of an antibody. In some embodiments, an antibody or antigen-binding fragment comprises an Fc region CH3 region of an antibody. In some embodiments, an antibody or antigen-binding fragment comprises an Fc region, a CHi region, and a kappa/lambda region from the constant domain of an antibody. In some embodiments, an antibody or antigen-binding fragment comprises a constant region, e.g., a heavy chain constant region and/or a light chain constant region. In some embodiments, such a constant region is modified compared to a wild-type constant region. That is, the polypeptide may comprise alterations or modifications to one or more of the three heavy chain constant domains (CHi, CH₂, or CH3) and/or to the light chain constant region domain (CL). Example modifications include additions, deletions, or substitutions of one or more amino acids in one or more domains. Such changes may be included to optimize effector function, half-life, etc.

“Internalizing” as used herein in reference to an antibody or antigen-binding fragment refers to an antibody or antigen-binding fragment that is capable of being taken through the cell's lipid bilayer membrane to an internal compartment (i.e., “internalized”) upon binding to the cell, preferably into a degradative compartment in the cell. For example, an internalizing anti-HER2 antibody is one that is capable of being taken into the cell after binding to HER2 on the cell membrane. In some embodiments, the antibody or antigen-binding fragment used in the ADCs disclosed herein targets a cell surface antigen (e.g., PCAD, HER2, CD48, CD74, EphA2, MET, TROP2, B7-H3, or 5T4) and is an internalizing antibody or internalizing antigen-binding fragment (i.e., the ADC transfers through the cellular membrane after antigen binding). In some embodiments, the internalizing antibody or antigen-binding fragment binds a receptor on the cell surface. An internalizing antibody or internalizing antigen-binding fragment that targets a receptor on the cell membrane may induce receptor-mediated endocytosis. In some embodiments, the internalizing antibody or internalizing antigen-binding fragment is taken into the cell via receptor-mediated endocytosis.

“Non-internalizing” as used herein in reference to an antibody or antigen-binding fragment refers to an antibody or antigen-binding fragment that remains at the cell surface upon binding to the cell. In some embodiments, the antibody or antigen-binding fragment used in the ADCs disclosed herein targets a cell surface antigen and is a non-internalizing antibody or non-internalizing antigen-binding fragment (i.e., the ADC remains at the cell surface and does not transfer through the cellular membrane after antigen binding). In some embodiments, the non-internalizing antibody or antigen-binding fragment binds a non-internalizing receptor or other cell surface antigen. Exemplary non-internalizing cell surface antigens include but are not limited to CA125 and CEA, and antibodies that bind to non-internalizing antigen targets are also known in the art (see, e.g., Bast et al. (1981) J Clin Invest. 68(5):1331-7; Scholler and Urban (2007) Biomark Med. 1(4):513-23; and Boudousq et al. (2013) PLoS One 8(7):e69613).

The term “EPH receptor A2” or “EphA2” as used herein, refers to any native form (also known as ephrin type-A receptor 2) of human EphA2. The term encompasses full-length human EphA2 (e.g., NCBI Reference Sequence: NP_004422.2; SEQ ID NO: 337), as well as any form of human EphA2 that may result from cellular processing. The term also encompasses functional variants or fragments of human EphA2, including but not limited to splice variants, allelic variants, and isoforms that retain one or more biologic functions of human EphA2 (i.e., variants and fragments are encompassed unless the context indicates that the term is used to refer to the wild-type protein only). EphA2 can be isolated from human, or may be produced recombinantly or by synthetic methods.

The term “anti-EphA2 antibody” or “antibody that binds to EphA2,” as used herein, refers to any form of antibody or antigen-binding fragment thereof that binds, e.g., specifically binds, to EphA2. The term encompasses monoclonal antibodies (including full-length monoclonal antibodies), polyclonal antibodies, and biologically functional antigen-binding fragments so long as they bind, e.g., specifically bind, to EphA2. WO 2007/030642 provides and is incorporated herein by reference for exemplary EphA2-binding sequences, including exemplary anti-EphA2 antibody sequences. In some embodiments, the anti-EphA2 antibody used in the ADCs disclosed herein is an internalizing antibody or internalizing antigen-binding fragment. 1C₁ (WO 2007/030642) is an exemplary anti-EphA2 antibody.

The term “P-cadherin” or “PCAD,” as used herein, refers to any native form of human PCAD (also known as cadherin 3, type 1 or CDH3). The term encompasses full-length human PCAD (e.g., UniProt Reference Sequence: P22223; SEQ ID NO:74), as well as any form of human PCAD that may result from cellular processing. The term also encompasses functional variants or fragments of human PCAD, including but not limited to splice variants, allelic variants, and isoforms that retain one or more biologic functions of human PCAD (i.e., variants and fragments are encompassed unless the context indicates that the term is used to refer to the wild-type protein only). PCAD can be isolated from human, or may be produced recombinantly or by synthetic methods.

The term “anti-PCAD antibody” or “antibody that binds to PCAD,” as used herein, refers to any form of antibody or antigen-binding fragment thereof that binds, e.g., specifically binds, to PCAD. The term encompasses monoclonal antibodies (including full-length monoclonal antibodies), polyclonal antibodies, and biologically functional antigen-binding fragments so long as they bind, e.g., specifically bind, to PCAD. WO 2016/203432 provides and is incorporated herein by reference for exemplary PCAD-binding sequences, including exemplary anti-PCAD antibody sequences. In some embodiments, the anti-PCAD antibody used in the ADCs disclosed herein is an internalizing antibody or internalizing antigen-binding fragment. NOV169N31Q (WO 2016/203432) is an exemplary anti-PCAD antibody.

The term “human epidermal growth factor receptor 2,” “HER2,” or “HER2/NEU,” as used herein, refers to any native form of human HER2. The term encompasses full-length human HER2 (e.g., UniProt Reference Sequence: P04626; SEQ ID NO:75), as well as any form of human HER2 that may result from cellular processing. The term also encompasses functional variants or fragments of human HER2, including but not limited to splice variants, allelic variants, and isoforms that retain one or more biologic functions of human HER2 (i.e., variants and fragments are encompassed unless the context indicates that the term is used to refer to the wild-type protein only). HER2 can be isolated from human, or may be produced recombinantly or by synthetic methods.

The term “anti-HER2 antibody” or “antibody that binds to HER2,” as used herein, refers to any form of antibody or antigen-binding fragment thereof that binds, e.g., specifically binds, to HER2. The term encompasses monoclonal antibodies (including full-length monoclonal antibodies), polyclonal antibodies, and biologically functional antigen-binding fragments so long as they bind, e.g., specifically bind, to HER2. U.S. Pat. Nos. 5,821,337 and 6,870,034 provide and are incorporated herein by reference for exemplary HER2-binding sequences, including exemplary anti-HER2 antibody sequences. In some embodiments, the anti-HER2 antibody used in the ADCs disclosed herein is an internalizing antibody or internalizing antigen-binding fragment. Trastuzumab (U.S. Pat. Nos. 5,821,337 and 6,870,034; see also Molina et al. (2001) Cancer Res. 61(12):4744-9) is an exemplary anti-HER2 antibody.

The term “cluster of differentiation 48” or “CD48,” as used herein, refers to any native form of human CD48 (also known as B-lymphocyte activation marker (BLAST-1) or signaling lymphocytic activation molecule 2 (SLAMF2)). The term encompasses full-length human CD48 (e.g., UniProt Reference Sequence: P09326; SEQ ID NO:77), as well as any form of human CD48 that may result from cellular processing. The term also encompasses functional variants or fragments of human CD48, including but not limited to splice variants, allelic variants, and isoforms that retain one or more biologic functions of human CD48 (i.e., variants and fragments are encompassed unless the context indicates that the term is used to refer to the wild-type protein only). CD48 can be isolated from human, or may be produced recombinantly or by synthetic methods.

The term “anti-CD48 antibody” or “antibody that binds to CD48,” as used herein, refers to any form of antibody or antigen-binding fragment thereof that binds, e.g., specifically binds, to CD48. The term encompasses monoclonal antibodies (including full-length monoclonal antibodies), polyclonal antibodies, and biologically functional antigen-binding fragments so long as they bind, e.g., specifically bind, to CD48. International Patent App. Nos. PCT/IB2021/060871, PCT/US2021/060560, and PCT/US2021/060620 provide and are incorporated herein by reference for exemplary CD48-binding sequences, including exemplary anti-CD48 antibody sequences. In some embodiments, the anti-CD48 antibody used in the ADCs disclosed herein is an internalizing antibody or internalizing antigen-binding fragment. SGN-CD48A (MEM102) and NY920 are exemplary anti-CD48 antibodies.

The term “cluster of differentiation 74” or “CD74,” as used herein, refers to any native form of human CD74 (also known as HLA class II histocompatibility antigen gamma chain or HLA-DR antigens-associated invariant chain). The term encompasses full-length human CD74 (e.g., NCBI Reference Sequence: NP_001020330.1; SEQ ID NO:140), as well as any form of human CD74 that may result from cellular processing. The term also encompasses functional variants or fragments of human CD74, including but not limited to splice variants, allelic variants, and isoforms that retain one or more biologic functions of human CD74 (i.e., variants and fragments are encompassed unless the context indicates that the term is used to refer to the wild-type protein only). CD74 can be isolated from human, or may be produced recombinantly or by synthetic methods.

The term “anti-CD74 antibody” or “antibody that binds to CD74,” as used herein, refers to any form of antibody or antigen-binding fragment thereof that binds, e.g., specifically binds, to CD74. The term encompasses monoclonal antibodies (including full-length monoclonal antibodies), polyclonal antibodies, and biologically functional antigen-binding fragments so long as they bind, e.g., specifically bind, to CD74. WO2020/236817 provides and is incorporated herein by reference for exemplary CD74-binding sequences, including exemplary anti-CD74 antibody sequences. In some embodiments, the anti-CD74 antibody used in the ADCs disclosed herein is an internalizing antibody or internalizing antigen-binding fragment. Milatuzumab (WO2003/074567) and VHmil x VKlaNQ (WO2020/236817) are an exemplary anti-CD74 antibodies.

The term “binding specificity,” as used herein, refers to the ability of an individual antibody or antigen binding fragment to preferentially react with one antigenic determinant over a different antigenic determinant. The degree of specificity indicates the extent to which an antibody or fragment preferentially binds to one antigenic determinant over a different antigenic determinant. Also, as used herein, the term “specific,” “specifically binds,” and “binds specifically” refers to a binding reaction between an antibody or antigen-binding fragment (e.g., an anti-HER2 antibody) and a target antigen (e.g., HER2) in a heterogeneous population of proteins and other biologics. Antibodies can be tested for specificity of binding by comparing binding to an appropriate antigen to binding to an irrelevant antigen or antigen mixture under a given set of conditions. If the antibody binds to the appropriate antigen with at least 2, 5, 7, 10 or more times more affinity than to the irrelevant antigen or antigen mixture, then it is considered to be specific. A “specific antibody” or a “target-specific antibody” is one that only binds the target antigen (e.g., PCAD, HER2, CD48, CD74, EphA2, MET, TROP2, B7-H3, or 5T4), but does not bind (or exhibits minimal binding) to other antigens. In some embodiments, an antibody or antigen-binding fragment that specifically binds a target antigen (e.g., PCAD, HER2, CD48, CD74, EphA2, MET, TROP2, B7-H3, or 5T4) has a K_{D} of less than 1×10⁶ M, less than 1×10⁻⁷ M, less than 1×10⁻⁸ M, less than 1×10⁻⁹ M, less than 1×10¹⁰ M, less than 1×10” M, less than 1×10¹² M, or less than 1×10³ M. In some embodiments, the K_{D} is 1 pM to 500 pM. In some embodiments, the K_{D} is between 500 pM to 1 PM, 1 pM to 100 nM, or 100 mM to 10 nM.

The term “affinity,” as used herein, refers to the strength of interaction between antibody and antigen at single antigenic sites. Without being bound by theory, within each antigen binding site, the variable region of the antibody “arm” interacts through weak non-covalent forces with the antigen at numerous sites; the more interactions, typically the stronger the affinity. The binding affinity of an antibody is the sum of the attractive and repulsive forces operating between the antigenic determinant and the binding site of the antibody.

The term “kₒₙ” or “ka” refers to the on-rate constant for association of an antibody to the antigen to form the antibody/antigen complex. The rate can be determined using standard assays, such as a surface plasmon resonance, biolayer inferometry, or ELISA assay.

The term “k_{0ff}” or “ka” refers to the off-rate constant for dissociation of an antibody from the antibody/antigen complex. The rate can be determined using standard assays, such as a surface plasmon resonance, biolayer inferometry, or ELISA assay.

The term “K_{D}” refers to the equilibrium dissociation constant of a particular antibody-antigen interaction. K_{D} is calculated by kₐ/kd. The rate can be determined using standard assays, such as a surface plasmon resonance, biolayer inferometry, or ELISA assay.

The term “epitope” refers to the portion of an antigen capable of being recognized and specifically bound by an antibody (or antigen-binding fragment). Epitope determinants generally consist of chemically active surface groupings of molecules such as amino acids or carbohydrate or sugar side chains and can have specific three-dimensional structural characteristics, as well as specific charge characteristics. When the antigen is a polypeptide, epitopes can be formed from contiguous amino acids or noncontiguous amino acids juxtaposed by tertiary folding of the polypeptide. An epitope may be “linear” or “conformational.” Conformational and linear epitopes are distinguished in that the binding to the former but not the latter is lost in the presence of denaturing solvents. The epitope bound by an antibody (or antigen-binding fragment) may be identified using any epitope mapping technique known in the art, including X-ray crystallography for epitope identification by direct visualization of the antigen-antibody complex, as well as monitoring the binding of the antibody to fragments or mutated variations of the antigen, or monitoring solvent accessibility of different parts of the antibody and the antigen. Exemplary strategies used to map antibody epitopes include, but are not limited to, array-based oligo-peptide scanning, limited proteolysis, site-directed mutagenesis, high-throughput mutagenesis mapping, hydrogen-deuterium exchange, and mass spectrometry (see, e.g., Gershoni et al. (2007) BioDrugs 21:145-56; and Hager-Braun and Tomer (2005) Expert Rev Proteomics 2:745-56).

Competitive binding and epitope binning can also be used to determine antibodies sharing identical or overlapping epitopes. Competitive binding can be evaluated using a cross-blocking assay, such as the assay described in “Antibodies, A Laboratory Manual,” Cold Spring Harbor Laboratory, Harlow and Lane (1^{St} edition 1988, 2^{nd} edition 2014). In some embodiments, competitive binding is identified when a test antibody or binding protein reduces binding of a reference antibody or binding protein to a target antigen such as PCAD, HER2, CD48, CD74, EphA2, MET, TROP2, B7-H3, or 5T4 (e.g., a binding protein comprising CDRs and/or variable domains selected from those identified in Tables 3-5), by at least about 50% in the cross-blocking assay (e.g., 50%, 60%, 70%, 80%, 90%, 95%, 99%, 99.5%, or more, or any percentage in between), and/or vice versa. In some embodiments, competitive binding can be due to shared or similar (e.g., partially overlapping) epitopes, or due to steric hindrance where antibodies or binding proteins bind at nearby epitopes (see, e.g., Tzartos, Methods in Molecular Biology (Morris, ed. (1998) vol. 66, pp. 55-66)). In some embodiments, competitive binding can be used to sort groups of binding proteins that share similar epitopes. For example, binding proteins that compete for binding can be “binned” as a group of binding proteins that have overlapping or nearby epitopes, while those that do not compete are placed in a separate group of binding proteins that do not have overlapping or nearby epitopes.

As used herein, the terms “peptide,” “polypeptide,” and “protein” are used interchangeably to refer to a polymer of amino acid residues. The terms encompass amino acid polymers comprising two or more amino acids joined to each other by peptide bonds, amino acid polymers in which one or more amino acid residues is an artificial chemical mimetic of a corresponding naturally-occurring amino acid, as well as naturally-occurring amino acid polymers and non-naturally-occurring amino acid polymers. The terms include, for example, biologically active fragments, substantially homologous polypeptides, oligopeptides, homodimers, heterodimers, variants of polypeptides, modified polypeptides, derivatives, analogs, fusion proteins, among others. The terms also include natural peptides, recombinant peptides, synthetic peptides, or a combination thereof. Unless otherwise indicated, a particular polypeptide sequence also implicitly encompasses conservatively modified variants thereof.

A “recombinant” protein refers to a protein (e.g., an antibody) made using recombinant techniques, e.g., through the expression of a recombinant nucleic acid.

An “isolated” protein refers to a protein unaccompanied by at least some of the material with which it is normally associated in its natural state. For example, a naturally-occurring polynucleotide or polypeptide present in a living organism is not isolated, but the same polynucleotide or polypeptide separated from some or all of the coexisting materials in the living organism, is isolated. The definition includes the production of an antibody in a wide variety of organisms and/or host cells that are known in the art.

An “isolated antibody,” as used herein, is an antibody that has been identified and separated from one or more (e.g., the majority) of the components (by weight) of its source environment, e.g., from the components of a hybridoma cell culture or a different cell culture that was used for its production. In some embodiments, the separation is performed such that it sufficiently removes components that may otherwise interfere with the suitability of the antibody for the desired applications (e.g., for therapeutic use). Methods for preparing isolated antibodies are known in the art and include, without limitation, protein A chromatography, anion exchange chromatography, cation exchange chromatography, virus retentive filtration, and ultrafiltration.

As used herein, the term “variant” refers to a nucleic acid sequence or an amino acid sequence that differs from a reference nucleic acid sequence or amino acid sequence respectively, but retains one or more biological properties of the reference sequence. A variant may contain one or more amino acid substitutions, deletions, and/or insertions (or corresponding substitution, deletion, and/or insertion of codons) with respect to a reference sequence. Changes in a nucleic acid variant may not alter the amino acid sequence of a peptide encoded by the reference nucleic acid sequence, or may result in amino acid substitutions, additions, deletions, fusions, and/or truncations. In some embodiments, a nucleic acid variant disclosed herein encodes an identical amino acid sequence to that encoded by the unmodified nucleic acid or encodes a modified amino acid sequence that retains one or more functional properties of the unmodified amino acid sequence. Changes in the sequence of peptide variants are typically limited or conservative, so that the sequences of the unmodified peptide and the variant are closely similar overall and, in many regions, identical. In some embodiments, a peptide variant retains one or more functional properties of the unmodified peptide sequence. A variant and unmodified peptide can differ in amino acid sequence by one or more substitutions, additions, deletions in any combination.

A variant of a nucleic acid or peptide can be a naturally-occurring variant or a variant that is not known to occur naturally. Variants of nucleic acids and peptides may be made by mutagenesis techniques, by direct synthesis, or by other techniques known in the art. A variant does not necessarily require physical manipulation of the reference sequence. As long as a sequence contains a different nucleic acid or amino acid as compared to a reference sequence, it is considered a “variant” regardless of how it was synthesized. In some embodiments, a variant has high sequence identity (i.e., 60% nucleic acid or amino acid sequence identity or higher) as compared to a reference sequence. In some embodiments, a peptide variant encompasses polypeptides having amino acid substitutions, deletions, and/or insertions as long as the polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% amino acid sequence identity with a reference sequence, or with a corresponding segment (e.g., a functional fragment) of a reference sequence, e.g., those variants that also retain one or more functions of the reference sequence. In some embodiments, a nucleic acid variant encompasses polynucleotides having amino acid substitutions, deletions, and/or insertions as long as the polynucleotide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% nucleic acid sequence identity with a reference sequence, or with a corresponding segment (e.g., a functional fragment) of a reference sequence.

The term “conservatively modified variant” applies to both amino acid and nucleic acid sequences. For nucleic acid sequences, conservatively modified variants refer to those nucleic acids which encode identical or essentially identical amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence. For polypeptide sequences, conservatively modified variants include individual substitutions, deletions, or additions to a polypeptide sequence which result in the substitution of an amino acid with a chemically similar amino acid. Conservative substitutions providing functionally similar amino acids are well known in the art.

The term “conservative sequence modifications,” as used herein, refers to amino acid modifications that do not significantly affect or alter the binding characteristics of, e.g., an antibody or antigen-binding fragment containing the amino acid sequence. Such conservative modifications include amino acid substitutions, additions, and deletions. Modifications can be introduced into an antibody or antigen-binding fragment by standard techniques known in the art, such as, e.g., site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, one or more amino acid residues within an antibody can be replaced with other amino acid residues from the same side chain family and the altered antibody can be tested using the functional assays described herein.

The term “homologous” or “identity,” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions. For example, if half (e.g., five positions in a polymer ten subunits in length) of the positions in two sequences are matched or homologous, the two sequences are 50% homologous; if 90% of the positions (e.g., 9 of 10), are matched or homologous, the two sequences are 90% homologous.

Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. The percent identity between the two sequences is a function of the number of identical positions shared by the sequences, taking into account the number of gaps, and the length of each gap, which need to be introduced for optimal alignment of the two sequences. Generally, the amino acid identity or homology between proteins disclosed herein and variants thereof, including variants of target antigens (such as PCAD, HER2, CD48, CD74, EphA2, MET, TROP2, B7-H3, or 5T4) and variants of antibody variable domains (including individual variant CDRs), is at least 80% to the sequences depicted herein, e.g., identities or homologies of at least 85%, 90%, 91%, 92%, 93%, 94%, 95%, 96%, 97%, 98%, 99%, almost 100%, or 100%.

The comparison of sequences and determination of percent identity between two sequences can be accomplished using a mathematical algorithm. In some embodiments, the percent identity between two amino acid sequences is determined using the Needleman and Wunsch ((1970) J Mol Biol. 48:444-53) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. An exemplary set of parameters is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17) which has been incorporated into the ALIGN program (version 2.0), using a PAM120 weight residue table, a gap length penalty of 12 and a gap penalty of 4.

The term “agent” is used herein to refer to a chemical compound, a mixture of chemical compounds, a biological macromolecule, an extract made from biological materials, or a combination of two or more thereof. The term “therapeutic agent” or “drug” refers to an agent that is capable of modulating a biological process and/or has biological activity. The Bcl-xL inhibitors and the ADCs comprising them, as described herein, are exemplary therapeutic agents.

The term “chemotherapeutic agent” or “anti-cancer agent” is used herein to refer to all agents that are effective in treating cancer (regardless of mechanism of action). Inhibition of metastasis or angiogenesis is frequently a property of a chemotherapeutic agent. Chemotherapeutic agents include antibodies, biological molecules, and small molecules, and encompass the Bcl-xL inhibitors and ADCs comprising them, as described herein. A chemotherapeutic agent may be a cytotoxic or cytostatic agent. The term “cytostatic agent” refers to an agent that inhibits or suppresses cell growth and/or multiplication of cells. The term “cytotoxic agent” refers to a substance that causes cell death primarily by interfering with a cell's expression activity and/or functioning.

The term “antineoplastic payload” or “antineoplastic compound” as used herein, refers to a compound or compounds that slow or inhibit the division of cancerous cells or that kill the cancerous cells. Non-limiting examples of antineoplastic payloads include BH3 mimetic compounds (e.g., MCl-1 inhibitors, Bcl-xL inhibitors, or Bcl-2 inhibitors), topoisomerase 1 inhibitors (e.g., topotecan, exatecan, deruxtecan or SN-38) or anti-mitotic drugs (e.g., monomethyl auristatin E (MMAE) or a taxane). In one embodiment, the antineoplastic payload is a BH3 mimetic compound. In one embodiment, the antineoplastic payload is a topoisomerase 1 inhibitor. In one embodiment, the antineoplastic payload is an anti-mitotic drug.

The term “antineoplastic non-BH3 mimetic,” as used herein, refers to a compound or compounds that are not BH3 mimetics and slow or inhibit the division of cancerous cells or that kill the cancerous cells. Non-limiting examples of antineoplastic non-BH3 mimetic include topoisomerase 1 inhibitors (e.g., topotecan, exatecan, deruxtecan or SN-38) or anti-mitotic drugs (e.g., monomethyl auristatin E (MMAE) or a taxane). In one embodiment, the antineoplastic non-BH3 mimetic is a topoisomerase 1 inhibitor. In one embodiment, the antineoplastic non-BH3 mimetic is an anti-mitotic drug.

The term “BH3 mimetic,” as used herein refers to an agent capable of disrupting the interaction between the proapoptotic and antiapoptotic members of the Bcl-2 family and are potent inducers of apoptosis. Exemplary BH3 mimetic includes inhibitors of Bcl-2, Bcl-xL, Bcl-w and Mcl-1.

The term “myeloid cell leukemia 1” or “Mel-1,” as used herein, refers to any native form of human Mcl-1, an anti-apoptotic member of the Bcl-2 protein family. The term encompasses full-length human Mcl-1 (e.g., UniProt Reference Sequence: Q07820; SEQ ID NO:71), as well as any form of human Mcl-1 that may result from cellular processing. The term also encompasses functional variants or fragments of human Mcl-1, including but not limited to splice variants, allelic variants, and isoforms that retain one or more biologic functions of human Mcl-1 (i.e., variants and fragments are encompassed unless the context indicates that the term is used to refer to the wild-type protein only). Mcl-1 can be isolated from human, or may be produced recombinantly or by synthetic methods.

The term “inhibit” or “inhibition” or “inhibiting,” as used herein, means to reduce a biological activity or process by a measurable amount, and can include but does not require complete prevention or inhibition. In some embodiments, “inhibition” means to reduce the expression and/or activity of Bcl-xL and/or one or more upstream modulators or downstream targets thereof.

The term “Mel-1 inhibitor,” as used herein, refers to an agent capable of reducing the expression and/or activity of Mcl-1 and/or one or more upstream modulators or downstream targets thereof. Exemplary Mcl-1 modulators (including exemplary inhibitors of Mcl-1) are described in WO 2015/097123; WO 2016/207216; WO 2016/207217; WO 2016/207225; WO 2016/207226; WO 2017/125224; WO 2019/035899, WO 2019/035911, WO 2019/035914, WO 2019/035927, US 2019/0055264, WO 2016/033486, WO 2017/147410, WO 2018/183418, and WO 2017/182625, each of which are incorporated herein by reference as exemplary Mcl-1 modulators, including exemplary Mcl-1 inhibitors, that can be included as drug moieties in the disclosed ADCs. For example, exemplary Mcl-1 inhibitors that can be included as drug moieties in the disclosed ADCs are those of formula:

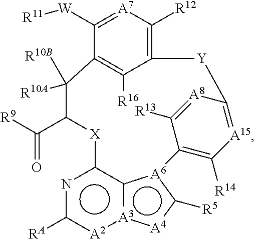

wherein each variable is defined as in WO2019/035911; WO 2019/035899; WO 2019/035914; or WO 2019/035927. Specific examples include, e.g.,

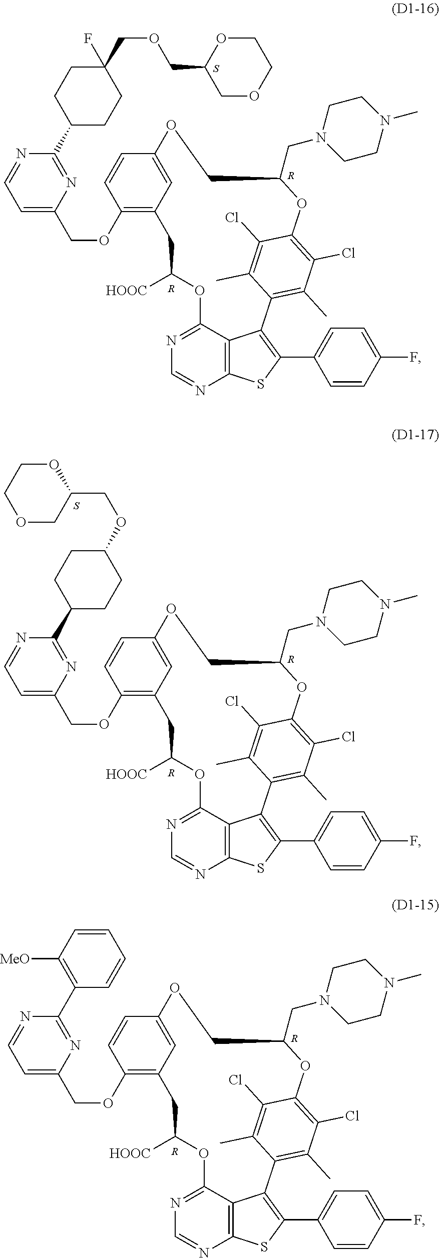

wherein each compound as a drug payload can be conjugated to an antibody or a linker via the nitrogen atom of the N-methyl in piperazinyl functional group of the compound. As used herein, the terms “derivative” and “analog” when referring to an Mcl-1 inhibitor, or the like, means any such compound that retains essentially the same, similar, or enhanced biological function or activity as compared to the original compound but has an altered chemical or biological structure.

As used herein, a “Mel-1 inhibitor drug moiety”, “Mel-1 inhibitor”, and the like refer to the component of an ADC or composition that provides the structure of an Mcl-1 inhibitor compound or a compound modified for attachment to an ADC that retains essentially the same, similar, or enhanced biological function or activity as compared to the original compound. In some embodiments, Mcl-1 inhibitor drug moiety is component (D¹ and/or D²) in an ADC of Formula (A). In some embodiments, the Mcl-1 inhibitor is represented by Formula (I) described herein

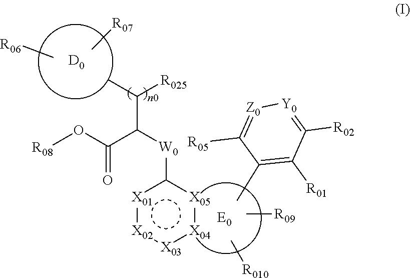

In some embodiments, the Mcl-1 inhibitor is a compound described in any one of the fiftieth through sixty-third embodiments in the summary section of the present disclosure.

The term “B-cell lymphoma-extra large” or “Bel-xL,” as used herein, refers to any native form of human Bel-xL, an anti-apoptotic member of the Bel-2 protein family. The term encompasses full-length human Bel-xL (e.g., UniProt Reference Sequence: Q07817-1; SEQ ID NO:71), as well as any form of human Bel-xL that may result from cellular processing. The term also encompasses functional variants or fragments of human Bel-xL, including but not limited to splice variants, allelic variants, and isoforms that retain one or more biologic functions of human Bel-xL (i.e., variants and fragments are encompassed unless the context indicates that the term is used to refer to the wild-type protein only). Bel-xL can be isolated from human, or may be produced recombinantly or by synthetic methods.

The term “Bel-xL inhibitor,” as used herein, refers to an agent capable of reducing the expression and/or activity of Bel-xL and/or one or more upstream modulators or downstream targets thereof. Exemplary Bel-xL modulators (including exemplary inhibitors of Bel-xL) are described in WO2010/080503, WO2010/080478, WO2013/055897, WO2013/055895, WO2016/094509, WO2016/094517, WO2016/094505, WO 2021/018858, WO 2021/018857, Tao et al., A CS Medicinal Chemistry Letters (2014), 5(10), 1088-109, and W ang et al., ACS Medicinal Chemistry Letters (2020), 11(10), 1829-1836, each of which are incorporated herein by reference as exemplary Bcl-xL modulators, including exemplary Bcl-xL inhibitors, that can be included as drug moieties in the disclosed ADCs.

As used herein, a “Bcl-xL inhibitor drug moiety”, “Bcl-xL inhibitor”, and the like refer to the component of an ADC or composition that provides the structure of a Bcl-xL inhibitor compound or a compound modified for attachment to an ADC that retains essentially the same, similar, or enhanced biological function or activity as compared to the original compound. In some embodiments, Bcl-xL inhibitor drug moiety is component (D¹ and/or D²) in an ADC of Formula (A). In some embodiments, the Bcl-xL inhibitor is represented by Formula (II) or Formula (III) described herein:

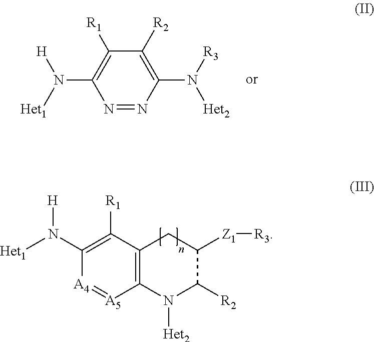

In some embodiments, the Bcl-xL inhibitor is a compound described in any one of the sixty-fourth through seventy-fourth embodiments in the summary section of the present disclosure.

The term “B-cell lymphoma 2” or “Bcl-2,” as used herein, refers to any native form of human Bcl-2, an anti-apoptotic member of the Bcl-2 protein family. The term encompasses full-length human Bcl-2 (e.g., UniProt Reference Sequence: P10415; SEQ ID NO:X), as well as any form of human Bcl-2 that may result from cellular processing. The term also encompasses functional variants or fragments of human Bcl-2, including but not limited to splice variants, allelic variants, and isoforms that retain one or more biologic functions of human Mcl-1 (i.e., variants and fragments are encompassed unless the context indicates that the term is used to refer to the wild-type protein only). Mcl-1 can be isolated from human, or may be produced recombinantly or by synthetic methods.

The term “Bcl-2 inhibitor,” as used herein, refers to an agent capable of reducing the expression and/or activity of Bcl-2 and/or one or more upstream modulators or downstream targets thereof. Exemplary Bcl-2 modulators (including exemplary inhibitors of Bcl-2) are described in WO 2013/110890, WO 2015/011400, WO 2015/011399, WO 2015/011397, WO 2015/011396, WO 2015/011164 and WO 2019081559, each of which are incorporated herein by reference as exemplary Bel-2 modulators, including exemplary Bcl-2 inhibitors, that can be included as drug moieties in the disclosed ADCs.

As used herein, a “Bcl-2 inhibitor drug moiety”, “Bcl-2 inhibitor”, and the like refer to the component of an ADC or composition that provides the structure of a Bcl-2 inhibitor compound or a compound modified for attachment to an ADC that retains essentially the same, similar, or enhanced biological function or activity as compared to the original compound. In some embodiments, Bcl-2 inhibitor drug moiety is component (D¹ and/or D²) in an ADC of Formula (A). In some embodiments, the Bcl-2 inhibitor is represented by Formula (IV) or Formula (V) described herein:

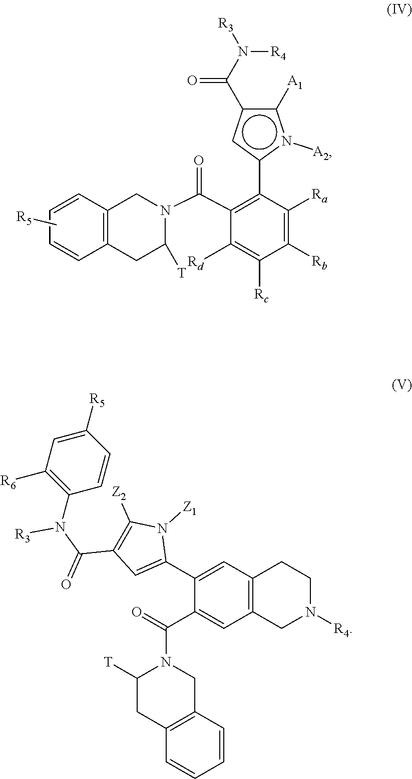

In some embodiments, the Bcl-2 inhibitor is a compound described in any one of the seventy-fifth through eighty-ninth embodiments in the summary section of the present disclosure.

The term “topoisomerase 1 inhibitor,” as used herein, refers to a compound or compounds which interferes with the action of topoisomerase 1 enzyme. In one embodiment such agents include, but are not limited to, topotecan, exatecan, deruxtecan or SN-38..

The term “anti-mitotic drug,” as used herein, refers to a compound or compounds which targets mitosis regulating enzymes, such as mircrotubule regulating enzymes, Polo-like Kinases (PLK), Kinesin-Spindle Protein (KSP), Aurora kinases, and the like. In one embodiment, an anti-mitotic drug is monomethyl auristatin E (MMAE) or a taxane. In some embodiments, taxane is selected from docetaxel, paclitaxel, or cabazitaxel.

The term “cancer,” as used herein, refers to the presence of cells possessing characteristics typical of cancer-causing cells, such as uncontrolled proliferation, immortality, metastatic potential, rapid growth and proliferation rate, and/or certain morphological features. Often, cancer cells can be in the form of a tumor or mass, but such cells may exist alone within a subject, or may circulate in the blood stream as independent cells, such as leukemic or lymphoma cells. The term “cancer” includes all types of cancers and cancer metastases, including hematological cancers, solid tumors, sarcomas, carcinomas and other solid and non-solid tumor cancers. Hematological cancers may include B-cell malignancies, cancers of the blood (leukemias), cancers of plasma cells (myelomas, e.g., multiple myeloma), or cancers of the lymph nodes (lymphomas). Exemplary B-cell malignancies include chronic lymphocytic leukemia (CLL), follicular lymphoma, mantle cell lymphoma, and diffuse large B-cell lymphoma. Leukemias may include acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMoL), etc. The terms “acute lymphoblastic leukemia” and “acute lymphocytic leukemia” can be used interchangeably to describe ALL. Lymphomas may include Hodgkin's lymphoma, non-Hodgkin's lymphoma, etc. Other hematologic cancers may include myelodysplasia syndrome (MDS). Solid tumors may include carcinomas such as adenocarcinoma, e.g., breast cancer, pancreatic cancer, prostate cancer, colon or colorectal cancer, lung cancer, gastric cancer, cervical cancer, endometrial cancer, ovarian cancer, cholangiocarcinoma, glioma, melanoma, etc. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

As used herein, the term “tumor” refers to any mass of tissue that results from excessive cell growth or proliferation, either benign or malignant, including precancerous lesions. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, pancreatic cancer, stomach cancer, colon cancer, head and neck cancer, or spleen cancer. In some embodiments, the tumor is a gastric cancer.

The terms “tumor cell” and “cancer cell” may be used interchangeably herein and refer to individual cells or the total population of cells derived from a tumor or cancer, including both non-tumorigenic cells and cancer stem cells. The terms “tumor cell” and “cancer cell” will be modified by the term “non-tumorigenic” when referring solely to those cells lacking the capacity to renew and differentiate to distinguish those cells from cancer stem cells.

The term “target-negative,” “target antigen-negative,” or “antigen-negative,” as used herein, refers to the absence of target antigen expression by a cell or tissue. The term “target-positive,” “target antigen-positive,” or “antigen-positive” refers to the presence of target antigen expression. For example, a cell or a cell line that does not express a target antigen may be described as target-negative, whereas a cell or cell line that expresses a target antigen may be described as target-positive.

The terms “subject” and “patient” are used interchangeably herein to refer to any human or non-human animal in need of treatment. Non-human animals include all vertebrates (e.g., mammals and non-mammals) such as any mammal. Non-limiting examples of mammals include humans, chimpanzees, apes, monkeys, cattle, horses, sheep, goats, swine, rabbits, dogs, cats, rats, mice, and guinea pigs. Non-limiting examples of non-mammals include birds and fish. In some embodiments, the subject is a human.

The term “a subject in need of treatment,” as used herein, refers to a subject that would benefit biologically, medically, or in quality of life from a treatment (e.g., a treatment with any one or more of the exemplary ADC compounds described herein).

As used herein, the term “treat,” “treating,” or “treatment” refers to any improvement of any consequence of disease, disorder, or condition, such as prolonged survival, less morbidity, and/or a lessening of side effects which result from an alternative therapeutic modality. In some embodiments, treatment comprises delaying or ameliorating a disease, disorder, or condition (i.e., slowing or arresting or reducing the development of a disease or at least one of the clinical symptoms thereof). In some embodiments, treatment comprises delaying, alleviating, or ameliorating at least one physical parameter of a disease, disorder, or condition, including those which may not be discernible by the patient. In some embodiments, treatment comprises modulating a disease, disorder, or condition, either physically (e.g., stabilization of a discernible symptom), physiologically (e.g., stabilization of a physical parameter), or both. In some embodiments, treatment comprises administration of a described ADC compound or composition to a subject, e.g., a patient, to obtain a treatment benefit enumerated herein. The treatment can be to cure, heal, alleviate, delay, prevent, relieve, alter, remedy, ameliorate, palliate, improve, or affect a disease, disorder, or condition (e.g., a cancer), the symptoms of a disease, disorder, or condition (e.g., a cancer), or a predisposition toward a disease, disorder, or condition (e.g., a cancer). In some embodiments, in addition to treating a subject having a disease, disorder, or condition, a composition disclosed herein can also be provided prophylactically to prevent or reduce the likelihood of developing that disease, disorder, or condition.

As used herein, the term “prevent”, “preventing,” or “prevention” of a disease, disorder, or condition refers to the prophylactic treatment of the disease, disorder, or condition; or delaying the onset or progression of the disease, disorder, or condition.

As used herein, a “pharmaceutical composition” refers to a preparation of a composition, e.g., an ADC compound or composition, in addition to at least one other (and optionally more than one other) component suitable for administration to a subject, such as a pharmaceutically acceptable carrier, stabilizer, diluent, dispersing agent, suspending agent, thickening agent, and/or excipient. The pharmaceutical compositions provided herein are in such form as to permit administration and subsequently provide the intended biological activity of the active ingredient(s) and/or to achieve a therapeutic effect. The pharmaceutical compositions provided herein preferably contain no additional components which are unacceptably toxic to a subject to which the formulation would be administered.

As used herein, the terms “pharmaceutically acceptable carrier” and “physiologically acceptable carrier,” which may be used interchangeably, refer to a carrier or a diluent that does not cause significant irritation to a subject and does not abrogate the biological activity and properties of the administered ADC compound or composition and/or any additional therapeutic agent in the composition. Pharmaceutically acceptable carriers may enhance or stabilize the composition or can be used to facilitate preparation of the composition. Pharmaceutically acceptable carriers can include solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. The carrier may be selected to minimize adverse side effects in the subject, and/or to minimize degradation of the active ingredient(s). An adjuvant may also be included in any of these formulations.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

The term “pharmaceutically acceptable salt,” as used herein, refers to a salt which does not abrogate the biological activity and properties of the compounds of the invention, and does not cause significant irritation to a subject to which it is administered. Examples of such salts include, but are not limited to: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes et al., “Commentary: Occurrence of Pharmaceutically Acceptable Anions and Cations in the Cambridge Structural Database,” J. Pharmaceutical Sciences, vol. 94, no. 10 (2005), and Berge et al., “Pharmaceutical Salts,” J. Pharmaceutical Sciences, vol. 66, no. 1 (1977), which are incorporated by reference herein.

In some embodiments, depending on their electronic charge, the antibody-drug conjugates (ADCs), linkers, payloads and linker-payloads described herein can contain a monovalent anionic counterion Mi. Any suitable anionic counterion can be used. In certain embodiments, the monovalent anionic counterion is a pharmaceutically acceptable monovalent anionic counterion. In certain embodiments, the monovalent anionic counterion Mᵢ- can be selected from bromide, chloride, iodide, acetate, trifluoroacetate, benzoate, mesylate, tosylate, triflate, formate, or the like. In some embodiments, the monovalent anionic counterion Mi- is trifluoroacetate or formate.

As used herein, the term “therapeutically effective amount” or “therapeutically effective dose,” refers to an amount of a compound described herein, e.g., an ADC compound or composition described herein, to effect the desired therapeutic result (i.e., reduction or inhibition of an enzyme or a protein activity, amelioration of symptoms, alleviation of symptoms or conditions, delay of disease progression, a reduction in tumor size, inhibition of tumor growth, prevention of metastasis). In some embodiments, a therapeutically effective amount does not induce or cause undesirable side effects. In some embodiments, a therapeutically effective amount induces or causes side effects but only those that are acceptable by a treating clinician in view of a patient's condition. In some embodiments, a therapeutically effective amount is effective for detectable killing, reduction, and/or inhibition of the growth or spread of cancer cells, the size or number of tumors, and/or other measure of the level, stage, progression and/or severity of a cancer. The term also applies to a dose that will induce a particular response in target cells, e.g., a reduction, slowing, or inhibition of cell growth. A therapeutically effective amount can be determined by first administering a low dose, and then incrementally increasing that dose until the desired effect is achieved. A therapeutically effective amount can also vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific amount may vary depending on, for example, the particular pharmaceutical composition, the subject and their age and existing health conditions or risk for health conditions, the dosing regimen to be followed, the severity of the disease, whether it is administered in combination with other agents, timing of administration, the tissue to which it is administered, and the physical delivery system in which it is carried. In the case of cancer, a therapeutically effective amount of an ADC may reduce the number of cancer cells, reduce tumor size, inhibit (e.g., slow or stop) tumor metastasis, inhibit (e.g., slow or stop) tumor growth, and/or relieve one or more symptoms.

As used herein, the term “prophylactically effective amount” or “prophylactically effective dose,” refers to an amount of a compound disclosed herein, e.g., an ADC compound or composition described herein, that is effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In some embodiments, a prophylactically effective amount can prevent the onset of disease symptoms, including symptoms associated with a cancer.

The term “p” or “drug loading” or “drug:antibody ratio” or “drug-to-antibody ratio” or “DAR″ refers to the number of drug moieties per antibody or antigen-binding fragment, i.e., drug loading, or the number of BH3 mimetic moieties per antibody or antigen-binding fragment (Ab) in ADCs of Formula (1). In ADCs comprising an antineoplastic compound (e.g. a BH3 mimetic drug moiety, a topoisomerase 1 inhibitor or an anti-mitotic drug ), “p” refers to the number of antineoplastic compounds (e.g. a BH3 mimetic drug moiety, a topoisomerase 1 inhibitor or an anti-mitotic drug ) linked to the antibody or antigen-binding fragment. In the present disclosure, one dual linker attaches two antineoplastic compounds (e.g. two BH3 mimetic drug moieties, or a BH3 mimetic and a non-BH3 mimetic (e.g., a topoisomerase 1 inhibitor or an anti-mitotic drug) to an antibody or antigen-binding fragment, therefore, p is 2 if the antibody or antigen-binding fragment only links with one dual linker having two antineoplastic compounds (e.g. two BH3 mimetic drug moieties, or a BH3 mimetic and a non-BH3 mimetic (e.g., a topoisomerase 1 inhibitor or an anti-mitotic drug) attached thereto. In compositions comprising multiple copies of ADCs of Formula (1), “average p” refers to the average number of antineoplastic compounds (e.g. two BH3 mimetic drug moieties, or a BH3 mimetic and a non-BH3 mimetic (e.g., a topoisomerase 1 inhibitor or an anti-mitotic drug) per antibody or antigen-binding fragment, also referred to as “average drug loading.”

### 1. Antibody-Drug Conjugates

The antibody-drug conjugate (ADC) compounds of the present disclosure include those with anti-cancer activity. In particular, the ADC compounds include an antibody or antigen-binding fragment conjugated (i.e., covalently attached by a dual linker) to two antineoplastic compounds, such as a BH3 mimetic drug moiety (e.g., a Mcl-1 inhibitor, a Bcl-2 inhibitor, or a Bcl-xL inhibitor or a combination thereof), a topoisomerase 1 inhibitor (e.g., topotecan, exatecan, deruxtecan or SN-38), or an anti-mitotic drug (e.g., monomethyl auristatin E (MMAE) or a taxane), wherein at least one antineoplastic compound is a BH3 mimetic drug moiety, and wherein the antineoplastic compound when not conjugated to an antibody or antigen-binding fragment has a cytotoxic or cytostatic effect. In some embodiments, the BH3 mimetic drug moiety when not conjugated to an antibody or antigen-binding fragment is capable of reducing the expression and/or activity of a Bcl-2 family protein (e.g., Mcl-1, Bcl-2 and/or Bcl-xL) and/or one or more upstream modulators or downstream targets thereof. Without being bound by theory, by targeting a Bcl-2 family protein (e.g., Mcl-1, Bcl-2 and/or Bcl-xL) expression and/or activity, in some embodiments, the ADCs disclosed herein may provide potent anti-cancer agents. Also, without being bound by theory, by conjugating the antineoplastic compound to an antibody that binds an antigen associated with expression in a tumor cell or cancer, the ADC may provide improved activity, better cytotoxic specificity, and/or reduced off-target killing as compared to the antineoplastic compound when administered alone.

In some embodiments, therefore, the components of the ADC are selected to (i) retain one or more therapeutic properties exhibited by the antibody and antineoplastic compounds in isolation, (ii) maintain the specific binding properties of the antibody or antigen-binding fragment; (iii) optimize drug loading and drug-to-antibody ratios; (iv) allow delivery, e.g., intracellular delivery, of the antineoplastic compound via stable attachment to the antibody or antigen-binding fragment; (v) retain ADC stability as an intact conjugate until transport or delivery to a target site; (vi) minimize aggregation of the ADC prior to or after administration; (vii) allow for the therapeutic effect, e.g., cytotoxic effect, of the antineoplastic compound after cleavage or other release mechanism in the cellular environment; (viii) exhibit in vivo anti-cancer treatment efficacy comparable to or superior to that of the antibody and antineoplastic compounds in isolation; (ix) minimize off-target killing by the antineoplastic compound; and/or (x) exhibit desirable pharmacokinetic and pharmacodynamics properties, formulatability, and toxicologic/immunologic profiles. Each of these properties may provide for an improved ADC for therapeutic use (Ab et al. (2015) Mol Cancer Ther. 14:1605-13).

The ADC compounds of the present disclosure may selectively deliver an effective dose of a cytotoxic or cytostatic agent to cancer cells or to tumor tissue. In some embodiments, the cytotoxic and/or cytostatic activity of the ADC is dependent on target antigen expression in a cell. In some embodiments, the disclosed ADCs are particularly effective at killing cancer cells expressing a target antigen while minimizing off-target killing. In some embodiments, the disclosed ADCs do not exhibit a cytotoxic and/or cytostatic effect on cancer cells that do not express a target antigen.

Exemplary BCMA-expressing cancers include but are not limited to multiple myeloma (Cho et al. (2018) Front Immunol. 9:1821).

Exemplary CD33-expressing cancers include but are not limited to colorectal cancer, pancreatic cancer, lymphoma, and leukemia (e.g., acute myeloid leukemia) (Human Protein Atlas; Walter (2014) Expert Opin Ther Targets 18(7):715-8).

Exemplary PCAD-expressing cancers include but are not limited to breast cancer, gastric cancer, endometrial cancer, ovarian cancer, pancreatic cancer, bladder cancer, prostate cancer, and melanoma (Vieira and Paredes (2015) Mol Cancer 14:178).

Exemplary HER2-expressing cancers include but are not limited to breast cancer, gastric cancer, bladder cancer, urothelial cell carcinoma, esophageal cancer, lung cancer (e.g., lung adenocarcinoma), uterine cancer (e.g., uterine serous endometrial carcinoma), salivary duct carcinoma, cervical cancer, endometrial cancer, and ovarian cancer (English et al. (2013) Mol Diagn Ther. 17:85-99).

Provided herein, in certain aspects, are ADC compounds comprising an antibody or antigen-binding fragment thereof (Ab) covalently linked to two antineoplastic payloads, such as a BH3 mimetic, a topoisomerase 1 inhibitor, or an anti-mitotic drug (D¹ and D²) through a dual linker (L), wherein at least one antineoplastic payload is a BH3 mimetic, and wherein the dual linker has one attachment point connected to the antibody and two attachment points to the two antineoplastic payloads, such as BH3 mimetics, and wherein the two antineoplastic payloads, such as BH3 mimetics, can be the same or different. In some embodiments, for the ADC compounds provided herein, the antibody or antigen-binding fragment thereof (Ab) targets a cancer cell. In some embodiments, the antibody or antigen-binding fragment is able to bind to a tumor-associated antigen (e.g., CD74, CD48, EphA2, PCAD, or HER2), e.g., with high specificity and high affinity. In some embodiments, the antibody or antigen-binding fragment is internalized into a target cell upon binding, e.g., into a degradative compartment in the cell. In some embodiments, the ADCs internalize upon binding to a target cell, undergo degradation, and release the Bcl-xL inhibitor drug moiety to kill cancer cells. The antineoplastic payloads, such as BH3 mimetics, topoisomerase 1 inhibitor, or anti-mitotic drug, may be released from the antibody and/or the linker moiety of the ADC by enzymatic action, hydrolysis, oxidation, or any other mechanism.

An exemplary ADC has Formula (1):

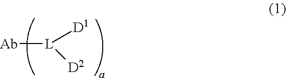

wherein Ab=an antibody or antigen-binding fragment, L=a dual linker moiety, D¹ and D²=a antineoplastic payload, such as BH3 mimetics, topoisomerase 1 inhibitor, or anti-mitotic drug, wherein at least one of D¹ and D² is a BH3 mimetics, and a=the number of antineoplastic payload,D¹ or D² attached per antibody or antigen-binding fragment.

### A. Antibodies

The antibody or antigen-binding fragment (Ab) of Formula (1) includes within its scope any antibody or antigen-binding fragment that specifically binds to a target antigen on a cell. In some embodiment, the antibody or antigen-binding fragment (Ab) of Formula (1) includes within its scope any antibody or antigen-binding fragment that specifically binds to a target antigen on a cancer cell. The antibody or antigen-binding fragment may bind to a target antigen with a dissociation constant (K_{D}) of <1 mM, <100 nM or <10 nM, or any amount in between, as measured by, e.g., BIAcore® analysis. In some embodiments, the K_{D} is 1 μM to 500 μM. In some embodiments, the K_{D} is between 500 μM to 1 μM, 1 μM to 100 nM, or 100 mM to 10 nM.

In some embodiments, the antibody or antigen-binding fragment is a four-chain antibody (also referred to as an immunoglobulin or a full-length or intact antibody), comprising two heavy chains and two light chains. In some embodiments, the antibody or antigen-binding fragment is an antigen-binding fragment of an immunoglobulin. In some embodiments, the antibody or antigen-binding fragment is an antigen-binding fragment of an immunoglobulin that retains the ability to bind a target cancer antigen and/or provide at least one function of the immunoglobulin.

In some embodiments, the antibody or antigen-binding fragment is an internalizing antibody or internalizing antigen-binding fragment thereof. In some embodiments, the internalizing antibody or internalizing antigen-binding fragment thereof binds to a target cancer antigen expressed on the surface of a cell and enters the cell upon binding. In some embodiments, the Bcl-xL inhibitor drug moiety of the ADC is released from the antibody or antigen-binding fragment of the ADC after the ADC enters and is present in a cell expressing the target cancer antigen (i.e., after the ADC has been internalized), e.g., by cleavage, by degradation of the antibody or antigen-binding fragment, or by any other suitable release mechanism.

In some embodiments, the antibodies comprise mutations that mediate reduced or no antibody-dependent cellular cytotoxicity (ADCC) or complement-dependent cytotoxicity (CDC). In some embodiments, these mutations are known as Fc Silencing, Fc Silent, or Fc Silenced mutations. In some embodiments, amino acid residues L234 and L235 of the IgGI constant region are substituted to A234 and A235 (also known as “LALA”). In some embodiments, amino acid residue N297 of the IgGI constant region is substituted to A297 (also known as “N297A”). In some embodiments, amino acid residues D265 and P329 of the IgGI constant region are substituted to A265 and A329 (also known as “DAPA”). Other antibody Fc silencing mutations may also be used. In some embodiments, the Fc silencing mutations are used in combination, for example D265A, N297A and P329A (also known as “DANAPA”).

As set forth herein, if modifications are made to the antibodies, they are further designated with that modification. For example if select amino acids in the antibody have been changed to cysteines (e.g. E152C, S375C according to EU numbering of the antibody heavy chain to facilitate conjugation to linker-drug moieties) they are designated as “CysMab”; or if the antibody has been modified with Fc silencing mutations D265A, N297A and P329A of the IgGI constant region according to EU numbering, “DANAPA” is added to the antibody name, or if the antibody has been modified with Fc silencing mutations D265A and P329A of the IgGI constant region according to EU numbering, “DAPA” is added to the antibody name.

Amino acid sequences of exemplary antibodies of the present disclosure, in addition to exemplary antigen targets, are set forth in Tables D1-D8.

TABLE D1
Antibodies Exemplified

| Antibody | | |
|---|---|---|
| Target | Antibody Code | mAb Reference |
| BCMA | BCMA or Ab B | J6M0 |
| CD33 | CD33ch or Ab G | MuMy9-6ch |
| CD33 | CD33 | gemtuzumab |
| PCAD | PCAD | NOV169N31Q |
| HER2/NEU | HER2 or Ab T | trastuzumab |
| HER2 | HER2 | disitamab |
| CD38 | CD38 | daratumumab |
| CD46 | CD46 | Anti-CD46 |
| CD48 | CD48 | SGN-CD48A |
| CD79b | CD79b | polatuzumab |
| EGFR | Ab C or EGFR1 CysMab | cetuximab |
| CD7 | Ab D | Anti-CD7 |
| TFRC | TFRC CysMab | CD71 (CX-2029) |
| EPCAM | EPCAM CysMab | oportuzumab |
| FOLR1 | FOLR1 CysMab | Mirvetuximab |
| ENPP3 | ENPP3 CysMab | ENPP3 (AGS16-7.8) |
| MET | MET CysMab | Telisotuzumab |
| MET | 9006 IgG1 CysMab | |
| MET | 9338 IgG1 CysMab | |
| MET | 9006 IgG2 CysMab | |
| MET | 9338 IgG2 CysMab | |
| MET | 8902 IgG1 CysMab | |
| MET | 8902 IgG2 CysMab | |
| AXL | AXL CysMab | Enapotamab |
| SLC34A2 | SLC34A2 CysMab | Lifastuzumab |
| NECTIN4 | NECTIN4 CysMab | Enfortumab |
| TACSTD2 | TACSTD2 CysMab | Sacituzumab |
| SLC39A6 | SLC39A6 CysMab | Ladiratuzumab |
| GPNMB | GPNMB CysMab | Glembatumumab |
| MSLN | MSLN CysMab | Anetumab |
| CD74 | CD74 CysMab | Milatuzumab |
| CD74 | VHmil × VK1aNQ | VHmil × VK1aNQ |
| F3/TF | F3 CysMab | Tisotumab |
| MUC16 | MUC16 CysMab | Sofituzumab |
| EGFR | EGFR2 CysMab | Aba |
| CD56 | CD56 CysMab | Lorvotuzumab |
| SEZ6 | SEZ6 CysMab | Anti-SEZ6 (Stemcentrx 17.46) |
| DLL3 | DLL3 CysMab | Rovalpituzumab |
| DLK1 | DLK1 CysMab | Anti-DLK1 (DI-2-14) |
| B7-H3 | B7-H3 CysMab | ABBV-155 |
| B7-H3 | B7-H3 | DS-5573a |
| IgG | IgG | anti-chiLysozyme (3207) |
| PCAD | PCAD CysMab | CQY679 |
| EphA2 | EphA2 CysMab | 1C1 |
| 5T4 | 5T4 Cysmab | |
| Trop2 | Trop2 Cysmab | Datopotamab |

TABLE D2
Amino acid sequences of mAb variable regions

| mAb | IgG chain | SEQ ID NO | Amino acid sequence |
|---|---|---|---|
| J6M0 | VH | 1 | QVQLVQSGAEVKKPGSSVKVSCKASGGTFS |
| | | | NYWMHWVRQAPGQGLEWMGATYRGHSDTYY |
| | | | NQKFKGRVTITADKSTSTAYMELSSLRSED |
| | | | TAVYYCARGAIYNGYDVLDNWGQGTLVTVS |
| | | | S |
| J6M0 | VL | 2 | DIQMTQSPSSLSASVGDRVTITCSASQDIS |
| | | | NYLNWYQQKPGKAPKLLIYYTSNLHSGVPS |
| | | | RFSGSGSGTDFTLTISSLQPEDFATYYCQQ |
| | | | YRKLPWTFGQGTKLEIK |
| MuMy9-6ch | VH | 3 | QVQLQQPGAEVVKPGASVKMSCKASGYTFT |
| | | | SYYIHWIKQTPGQGLEWVGVIYPGNDDISY |
| | | | NQKFKGKATLTADKSSTTAYMQLSSLTSED |
| | | | SAVYYCAREVRLRYFDVWGAGTTVTVSS |
| MuMy9-6ch | VL | 4 | NIMLTQSPSSLAVSAGEKVTMSCKSSQSVF |
| | | | FSSSQKNYLAWYQQIPGQSPKLLIYWASTR |
| | | | ESGVPDRFTGSGSGTDFTLIISSVQSEDLA |
| | | | IYYCHQYLSSRTFGGGTKLEIK |
| gemtuzumab | VH | 5 | EVQLVQSGAEVKKPGSSVKVSCKASGYTIT |
| | | | DSNIHWVRQAPGQSLEWIGYIYPYNGGTDY |
| | | | NQKFKNRATLTVDNPTNTAYMELSSLRSED |
| | | | TAFYYCVNGNPWLAYWGQGTLVTVSS |
| gemtuzumab | VL | 6 | DIQLTQSPSTLSASVGDRVTITCRASESLD |
| | | | NYGIRFLTWFQQKPGKAPKLLMYAASNQGS |
| | | | GVPSRFSGSGSGTEFTLTISSLQPDDFATY |
| | | | YCQQTKEVPWSFGQGTKVEVK |
| NOV169N31Q | VH | 7 | QVQLQQSGPGLVKPSQTLSLTCAISGDSVS |
| | | | SQSAAWNWIRQSPSRGLEWLGRIYYRSKWY |
| | | | NDYALSVKSRITINPDTSKNQFSLQLNSVT |
| | | | PEDTAVYYCARGEGYGREGFAIWGQGTLVT |
| | | | VSS |
| NOV169N31Q | VL | 8 | DIQMTQSPSSLSASVGDRVTITCRASQTIS |
| | | | NTLAWYQQKPGKAPKLLIYAASNLQSGVPS |
| | | | RFSGSGSGTDFTLTISSLQPEDFATYYCQQ |
| | | | YLSWFTFGQGTKVEIK |
| trastuzumab | VH | 9 | EVQLVESGGGLVQPGGSLRLSCAASGENIK |
| | | | DTYIHWVRQAPGKGLEWVARIYPTNGYTRY |
| | | | ADSVKGRFTISADTSKNTAYLQMNSLRAED |
| | | | TAVYYCSRWGGDGFYAMDYWGQGTLVTVSS |
| trastuzumab | VL | 10 | DIQMTQSPSSLSASVGDRVTITCRASQDVN |
| | | | TAVAWYQQKPGKAPKLLIYSASFLYSGVPS |
| | | | RFSGSRSGTDFTLTISSLQPEDFATYYCQQ |
| | | | HYTTPPTFGQGTKVEIKRT |
| daratumumab | VH | 11 | EVQLLESGGGLVQPGGSLRLSCAVSGFTFN |
| | | | SFAMSWVRQAPGKGLEWVSAISGSGGGTYY |
| | | | ADSVKGRFTISRDNSKNTLYLQMNSLRAED |
| | | | TAVYFCAKDKILWFGEPVFDYWGQGTLVTV |
| | | | SS |
| daratumumab | VL | 12 | EIVLTQSPATLSLSPGERATLSCRASQSVS |
| | | | SYLAWYQQKPGQAPRLLIYDASNRATGIPA |
| | | | RFSGSGSGTDFTLTISSLEPEDFAVYYCQQ |
| | | | RSNWPPTFGQGTKVEI |
| SGN-48A | VH | 13 | QVQLVQSGSELKKPGASVKVSCKASGYTFT |
| | | | DFGMNWVRQAPGQGLEWMGWINTFTGEPSY |
| | | | GNVFKGRFVFSLDTSVSTAYLQISSLKAED |
| | | | TAVYYCARRHGNGNVFDSWGQGTLVTVSS |
| SGN-48A | VL | 14 | EIVLTQSPDFQSVTPKEKVTITCRASQSIG |
| | | | SNIHWYQQKPDQSPKLLIKYTSESISGVPS |
| | | | RFSGSGSGTDFTLTINSLEAEDAATYYCQQ |
| | | | SNSWPLTFGGGTKVEIK |
| polatuzumab | VH | 80 | EVQLVESGGGLVQPGGSLRLSCAASGYTFS |
| | | | SYWIEWVRQAPGKGLEWIGEILPGGGDTNY |
| | | | NEIFKGRATFSADTSKNTAYLQMNSLRAED |
| | | | TAVYYCTRRVPIRLDYWGQGTLVTVSS |
| polatuzumab | VL | 81 | DIQLTQSPSSLSASVGDRVTITCKASQSVD |
| | | | YEGDSFLNWYQQKPGKAPKLLIYAASNLES |
| | | | GVPSRFSGSGSGTDFTLTISSLQPEDFATY |
| | | | YCQQSNEDPLTFGQGTKVEIK |
| Anti-CD46 | VH | 90 | QVQLVQSGGGVVQPGRSLRLACAASGLTVN |
| | | | NYAMHWVRQAPGKGLEWVAVISYDGNNKYY |
| | | | ADSVKGRFTISRDNSKNTLYLQ |
| | | | MNSLRAEDTAVYYCAKGGGYFDLWGRGTLV |
| | | | TVSS |
| Anti-CD46 | VL | 91 | QSVLTQPPSVSGAPGQRVTISCTGSSSNIG |
| | | | AGYDVHWYQQLPGTAPKLLIYGNNNRP |
| | | | SGVPDRFSGSKSGTSASLAI TGLQAEDEA |
| | | | DYYCSSYTSGTWLFG GGTKLTVL |
| Cetuximab | VH | 151 | EVQLQESGPGLVKPSQTLSLTCTVSGYSIS |
| | | | RDFAWNWIRQPPGKGLEWMGYISYNGNTRY |
| | | | QPSLKSRITISRDTSKNQFFLKLNSVTAAD |
| | | | TATYYCVTASRGFPYWGQGTLVTVSS |
| Cetuximab | VL | 152 | DIQMTQSPSSMSVSVGDRVTITCHSSQDIN |
| | | | SNIGWLQQKPGKSFKGLIYHGTNLDDGVPS |
| | | | RFSGSGSGTDYTLTISSLQPEDFATYYCVQ |
| | | | YAQFPWTFGGGTKLEIK |
| VHmi1 x | VH | 153 | QVQLQQSGSELKKPGASVKVSCKASGYTFT |
| VK1aNQ | | | NYGVNWIKQAPGQGLQWMGWINPNTGEPTF |
| | | | DDDFKGRFAFSLDTSVSTAYLQISSLKADD |
| | | | TAVYFCSRSRGKNEAWFAYWGQGTLVTVSS |
| VHmi1 x | VL | 154 | DIVMTQTPLSLPVTPGEPASISCRSSQSLV |
| VK1aNQ | | | HRNQNTYLHWYLQKPGQSPQLLIYTVSNRF |
| | | | SGVPDRFSGSGSGTDFTLKISRVEAEDVGV |
| | | | YFCSQSSHVPPTFGQGTKLEIK |
| SEZ6 | VH | 155 | QVQLVQSGAEVKKPGASVKVSCKASGYTFT |
| | | | SYWINWVRQAPGQGLEWIGNIFPDTTTTNY |
| | | | NEKFKGRVTLTRDTSISTAYMELSRLRSDD |
| | | | TAVYYCAREYYDGTYDAMDYWGQGTLVTVS |
| | | | S |
| SEZ6 | VL | 156 | AIQMTQSPSSLSASVGDRVTITCKASQSVN |
| | | | NDVAWYQQKPGKAPKLLIYYASNRYTGVPS |
| | | | RFSGSGSGTDFTLTISSLQPEDFATYFCQQ |
| | | | DYSSPRTFGQGTKLEIK |
| CD56 | VH | 157 | QVQLVESGGGVVQPGRSLRLSCAASGFTFS |
| | | | SFGMHWVRQAPGKGLEWVAYISSGSFTIYY |
| | | | ADSVKGRFTISRDNSKNTLYLQMNSLRAED |
| | | | TAVYYCARMRKGYAMDYWGQGTLVTVSS |
| CD56 | VL | 158 | DVVMTQSPLSLPVTLGQPASISCRSSQIII |
| | | | HSDGNTYLEWFQQRPGQSPRRLIYKVSNRF |
| | | | SGVPDRFSGSGSGTDFTLKISRVEAEDVGV |
| | | | YYCFQGSHVPHTFGQGTKVEIK |
| DLL3 | VH | 159 | QVQLVQSGAEVKKPGASVKVSCKASGYTFT |
| | | | NYGMNWVRQAPGQGLEWMGWINTYTGEPTY |
| | | | ADDFKGRVTMTTDTSTSTAYMELRSLRSDD |
| | | | TAVYYCARIGDSSPSDYWGQGTLVTVSS |
| DLL3 | VL | 160 | EIVMTQSPATLSVSPGERATLSCKASQSVS |
| | | | NDVVWYQQKPGQAPRLLIYYASNRYTGIPA |
| | | | RFSGSGSGTEFTLTISSLQSEDFAVYYCQQ |
| | | | DYTSPWTFGQGTKLEIK |
| DLK1 | VH | 161 | EVQLQQSGAELVKPGASVKLSCTASGENIR |
| | | | DTYIHWVKQRPEQGLEWIGRIDPPNGNLKY |
| | | | DPKFQGKATITADTSSNTAYLQFSSLTSED |
| | | | TAVYYCARSDGYSFAYWGQGTLVTVSS |
| DLK1 | VL | 162 | DIVMTQAAPSVPVTPGESVSISCRSSKSLL |
| | | | HSNGNTYLYWFLQRPGQSPQLLIYRMSNLA |
| | | | SGVPDRFSGSGSGTAFTLRISRVEAEDVGV |
| | | | YYCMQHVEYPFTFGSGTKLEIK |
| B7-H3 | VH | 163 | EVQLVQSGAEVKKPGSSVKVSCKASGYTFS |
| ABBV-155 | | | SYWMHWVRQAPGQGLEWIGLIHPESGSTNY |
| | | | NEMFKNRATLTVDRSTSTAYMELSSLRSED |
| | | | TAVYYCAGGGRLYFDYWGQGTTVTVSS |
| B7-H3 | VL | 164 | DIVMTQSPLSLPVTPGEPASISCRSSQSLV |
| ABBV-155 | | | HSNRDTYLRWYLQKPGQSPQLLIYKVSNRF |
| | | | SGVPDRFSGSGSGTDFTLKISRVEAEDVGV |
| | | | YYCSQSTHVPYTFGGGTKVEIK |
| B7-H3 | VH | 165 | QVQLVQSGAEVKKPGSSVKVSCKASGYTFT |
| DS-5573a | | | NYVMHWVRQAPGQGLEWMGYINPYNDDVKY |
| | | | NEKFKGRVTITADESTSTAYMELSSLRSED |
| | | | TAVYYCARWGYYGSPLYYFDYWGQGTLVTV |
| | | | SS |
| B7-H3 | VL | 166 | EIVLTQSPATLSLSPGERATLSCRASSRLI |
| DS-5573a | | | YMHWYQQKPGQAPRPLIYATSNLASGIPAR |
| | | | FSGSGSGTDFTLTISSLEPEDFAVYYCQQW |
| | | | NSNPPTFGQGTKVEIK |
| IgG | VH | 167 | QVQLQQSGPGLVKPSQTLSLTCAISGDSVS |
| | | | SNSAAWSWIRQSPGRGLEWLGRIYYRSKWY |
| | | | NDYAVSVKSRITINPDTSKNQFSLQLNSVT |
| | | | PEDTAVYYCARLDHRYHEDTVYPGMDVWGQ |
| | | | GTLVTVSS |
| IgG | VL | 168 | DIELTQPPSVSVAPGQTARISCSGDNLPAY |
| | | | TVTWYQQKPGQAPVLVIYDDSDRPSGIPER |
| | | | FSGSNSGNTATLTISGTQAEDEADYYCASW |
| | | | DPSSGVVFGGGTKLTVL |
| HER2 | VH | 437 | EVQLVQSGAEVKKPGATVKISCKVSGYTFT |
| Disitamab | | | DYYIHWVQQAPGKGLEWMGRVNPDHGDSYY |
| | | | NQKFKDKATITADKSTDTAYMELSSLRSED |
| | | | TAVYFCARNYLFDHWGQGTLVTVSS |
| HER2 | VL | 338 | DIQMTQSPSSVSASVGDRVTITCKASQDVG |
| Disitamab | | | TAVAWYQQKPGKAPKLLIYWASIRHTGVPS |
| | | | RFSGSGSGTDFTLTISSLQPEDFATYYCHQ |
| | | | FATYTFGGGTKVEIK |
| 9006 | VH | 339 | QVQLVQSGSELKKPGASVKVSCKASGYTFT |
| | | | NFRMNWVKQAPGQGLKWMGWINTYTGEPTY |
| | | | VDDLKGRFVFSLDTSVSTAYLQISSLKAED |
| | | | TAVYYCARKGIARAMDYWGQGTTVTVSS |
| 9006 | VL | 340 | DIVMTQSPDSLAVSLGERATINCKSSQSLL |
| | | | DSGNQKNYLAWYQQKPGQPPKLLIFGASTR |
| | | | ESGVPDRFSGSGSGTDFTLTISSLQAEDVA |
| | | | VYYCQNDHSYPYTFGQGTKLEIK |
| 9338 | VH | 341 | QVQLVQSGAEVKKPGSSVKVSCKASGYTFT |
| | | | SYWMHWVRQAPGQGLEWMGYINPSSGHIEN |
| | | | NQKFKDRVTITADKSTSTAYMELSSLRSED |
| | | | TAVYYCARGRFAYWGQGTLVTVSS |
| 9338 | VL | 342 | EIVLTQSPATLSLSPGERATLSCSASSSVS |
| | | | SGYLYWYQQKPGQAPRLLIYSTSNLASGIP |
| | | | ARFSGSGSGTDFTLTISSLEPEDFAVYYCH |
| | | | QWSSYPFTFGSGTKLEIK |
| 8902 | VH | 343 | QVQLQESGPGLVKPSQTLSLTCTVSGFSLT |
| | | | DYGVSWIRQPPGKGLEWIGVIWGGGSTYHN |
| | | | SALKSRVTISVDTSKNQFSLKLSSVTAADT |
| | | | AVYYCAKTSYDGYYFDYWGQGTLVTVSS |
| 8902 | VL | 344 | EIVLTQSPATLSLSPGERATLSCSASSSIN |
| | | | NMHWYQQKPGQAPRLLIYDTSKLASGIPAR |
| | | | FSGSGSGTDFTLTISSLEPEDFAVYYCQQW |
| | | | SFNPLTFGQGTKLEIK |

TABLE D2a
Nucleotide sequence corresponding to the variable domain heavy
and light chain (VH and VL) amino acid sequences
of the HER2 Disitamab and 8902 antibody

| mAb | IgG chain | SEQ ID NO | Nucleotide sequence |
|---|---|---|---|
| HER2 | VH | 345 | gaagttcagctggttcagtct |
| Disitamab | | | ggcgccgaagtgaagaaacct |
| | | | ggcgccaccgtgaagatcagc |
| | | | tgcaaggtgtccggctacacc |
| | | | ttcaccgactactacatccac |
| | | | tgggtgcagcaggcccctggc |
| | | | aaaggacttgagtggatgggc |
| | | | agagtgaaccccgatcacggc |
| | | | gacagctactacaaccagaag |
| | | | ttcaaggacaaggccaccatc |
| | | | accgccgacaagagcaccgat |
| | | | accgcctacatggaactgagc |
| | | | agcctgagaagcgaggatacc |
| | | | gccgtgtacttctgcgcccgg |
| | | | aactacctgtttgaccactgg |
| | | | ggacagggcaccctggtcaca |
| | | | gttagttct |
| HER2 | VL | 346 | gacattcagatgacacagagc |
| Disitamab | | | cctagcagcgtgtccgcctct |
| | | | gtgggagacagagtgaccatc |
| | | | acatgcaaggccagccaggat |
| | | | gtgggaacagccgtggcttgg |
| | | | tatcagcagaagcctggcaag |
| | | | gcccctaagctgctgatctac |
| | | | tgggccagcatcagacacaca |
| | | | ggcgtgcccagcagattttct |
| | | | ggcagcggctctggcaccgac |
| | | | ttcaccctgaccatatctagc |
| | | | ctgcagccagaggacttcgcc |
| | | | acctactactgccaccagttt |
| | | | gccacctacaccttcggcgga |
| | | | ggcaccaaggtggaaatcaag |
| 8902 | VH | 347 | caggtgcagctgcaggaatctgg |
| | | | acctggcctcgtgaagccctccc |
| | | | agaccctgtctctgacctgcacc |
| | | | gtgtccggcttctccctgaccga |
| | | | ttacggcgtgtcctggatcagac |
| | | | agccccctggcaagggcctggaa |
| | | | tggatcggagtgatctggggcgg |
| | | | aggctccacctaccacaactccg |
| | | | ccctgaagtccagagtgaccatc |
| | | | tccgtggacacctccaagaacca |
| | | | gttcagcctgaagctgtcctccg |
| | | | tgaccgccgctgataccgccgtg |
| | | | tactactgegccaagacctccta |
| | | | cgacggctactacttcgactact |
| | | | ggggccagggcaccctcgtgacc |
| | | | gtgtcatct |
| 8902 | VL | 348 | gagatcgtgctgacccagtctcc |
| | | | tgccaccctgtctctgagccctg |
| | | | gcgagagagctaccctgtcctgc |
| | | | tccgcctcctcctccatcaacaa |
| | | | catgcactggtatcagcagaagc |
| | | | ccggccaggcccccagactgctg |
| | | | atctacgacacctccaagctggc |
| | | | ctccggcatccctgccagattct |
| | | | ccggctctggctctggcaccgac |
| | | | tttaccctgaccatctccagcct |
| | | | ggaacccgaggacttcgccgtgt |
| | | | actactgccagcagtggtccttc |
| | | | aaccccctgacctttggccaggg |
| | | | caccaagctggaaatcaag |

TABLE D3
Amino acid sequences of mAb CDRs (Combined)

| | | SEQ | |
|---|---|---|---|
| | IgG | ID | |
| mAb | chain | NO | Amino acid sequence |
| J6M0 | HCDR1 | 15 | GGTFSNYWMH |
| J6M0 | HCDR2 | 16 | ATYRGHSDTYYNQKFKG |
| J6M0 | HCDR3 | 17 | GAIYNGYDVLDN |
| J6M0 | LCDR1 | 18 | SASQDISNYLN |
| J6M0 | LCDR2 | 19 | YTSNLHS |
| J6M0 | LCDR3 | 20 | QQYRKLPWT |
| MuMy9-6ch | HCDR1 | 21 | GYTFTSYYIH |
| MuMy9-6ch | HCDR2 | 22 | VIYPGNDDISYNQKFKG |
| MuMy9-6ch | HCDR3 | 23 | EVRLRYFDV |
| MuMy9-6ch | LCDR1 | 24 | KSSQSVFFSSSQKNYLA |
| MuMy9-6ch | LCDR2 | 25 | WASTRES |
| MuMy9-6ch | LCDR3 | 26 | HQYLSSRT |
| gemtuzumab | HCDR1 | 27 | GYTITDSNIH |
| gemtuzumab | HCDR2 | 28 | YIYPYNGGTDYNQKFKN |
| gemtuzumab | HCDR3 | 29 | GNPWLAY |
| gemtuzumab | LCDR1 | 30 | RASESLDNYGIRFLT |
| gemtuzumab | LCDR2 | 31 | AASNQGS |
| gemtuzumab | LCDR3 | 32 | QQTKEVPWS |
| NOV169N31Q | HCDR1 | 33 | TCAISGDSVSSQSAAWN |
| NOV169N31Q | HCDR2 | 34 | RIYYRSKWYNDYALSVKS |
| NOV169N31Q | HCDR3 | 35 | GEGYGREGFAI |
| NOV169N31Q | LCDR1 | 36 | RASQTISNTLA |
| NOV169N31Q | LCDR2 | 37 | AASNLQS |
| NOV169N31Q | LCDR3 | 38 | QQYLSWFT |
| trastuzumab | HCDR1 | 39 | GFNIKDTYIH |
| trastuzumab | HCDR2 | 40 | RIYPTNGYTRYADSVKG |
| trastuzumab | HCDR3 | 41 | WGGDGFYAMDV |
| trastuzumab | LCDR1 | 42 | RASQDVNTAVAW |
| trastuzumab | LCDR2 | 43 | SASFLES |
| trastuzumab | LCDR3 | 44 | QQHYTTPPT |
| daratumumab | HCDR1 | 45 | GFTFNSFAMS |
| daratumumab | HCDR2 | 46 | AISGSGGGTYYADSVKG |
| daratumumab | HCDR3 | 47 | DKILWFGEPVEDY |
| daratumumab | LCDR1 | 48 | RASQSVSSYLA |
| daratumumab | LCDR2 | 49 | DASNRAT |
| daratumumab | LCDR3 | 50 | QQRSNWPPT |
| SGN-48A | HCDR1 | 51 | GYTFTDFGMN |
| SGN-48A | HCDR2 | 52 | WINTFTGEPSYGNVFKG |
| SGN-48A | HCDR3 | 53 | RHGNGNVFDS |
| SGN-48A | LCDR1 | 54 | RASQSIGSNIH |
| SGN-48A | LCDR2 | 55 | YTSESIS |
| SGN-48A | LCDR3 | 56 | QQSNSWPLT |
| polatuzumab | HCDR1 | 82 | GYTFSSYWIE |
| polatuzumab | HCDR2 | 83 | EILPGGGDTNYNEIFKG |
| polatuzumab | HCDR3 | 84 | RVPIRLDY |
| polatuzumab | LCDR1 | 85 | ITCKASQSVDYEGDSFLN |
| polatuzumab | LCDR2 | 86 | AASNLES |
| polatuzumab | LCDR3 | 87 | QQSNEDPLT |
| VHmi1 x VK1aNQ | HCDR1 | 169 | GYTFTNYGVN |
| VHmi1 x VK1aNQ | HCDR2 | 170 | WINPNTGEPTFDDDFKG |
| VHmi1 x VK1aNQ | HCDR3 | 171 | SRGKNEAWFAY |
| VHmi1 x VK1aNQ | LCDR1 | 172 | RSSQSLVHRNQNTYLH |
| VHmi1 x VK1aNQ | LCDR2 | 173 | TVSNRFS |
| VHmi1 x VK1aNQ | LCDR3 | 174 | SQSSHVPPT |
| SEZ6 | HCDR1 | 175 | GYTFTSYWIN |
| SEZ6 | HCDR2 | 176 | NIFPDTTTTNYNEKFKG |
| SEZ6 | HCDR3 | 177 | EYYDGTYDAMDY |
| SEZ6 | LCDR1 | 178 | KASQSVNNDVA |
| SEZ6 | LCDR2 | 179 | YASNRYT |
| SEZ6 | LCDR3 | 180 | QQDYSSPRT |
| CD56 | HCDR1 | 181 | GFTFSSFGMH |
| CD56 | HCDR2 | 182 | YISSGSFTIYYADSVKG |
| CD56 | HCDR3 | 183 | MRKGYAMDY |
| CD56 | LCDR1 | 184 | RSSQIIIHSDGNTYLE |
| CD56 | LCDR2 | 185 | KVSNRFS |
| CD56 | LCDR3 | 186 | FQGSHVPHT |
| DLL3 | HCDR1 | 187 | GYTFTNYGMN |
| DLL3 | HCDR2 | 188 | WINTYTGEPTYADDFKG |
| DLL3 | HCDR3 | 189 | IGDSSPSDY |
| DLL3 | LCDR1 | 190 | KASQSVSNDVV |
| DLL3 | LCDR2 | 179 | YASNRYT |
| DLL3 | LCDR3 | 191 | QQDYTSPWT |
| DLK1 | HCDR1 | 192 | GFNIRDTYIH |
| DLK1 | HCDR2 | 193 | RIDPPNGNLKYDPKFQG |
| DLK1 | HCDR3 | 194 | SDGYSFAY |
| DLK1 | LCDR1 | 195 | RSSKSLLHSNGNTYLY |
| DLK1 | LCDR2 | 196 | RMSNLAS |
| DLK1 | LCDR3 | 197 | MQHVEYPFT |
| B7-H3 | HCDR1 | 198 | GYTFSSYWMH |
| ABBV-155 | | | |
| B7-H3 | HCDR2 | 199 | LIHPESGSTNYNEMFKN |
| ABBV-155 | | | |
| B7-H3 | HCDR3 | 200 | GGRLYFDY |
| ABBV-155 | | | |
| B7-H3 | LCDR1 | 201 | RSSQSLVHSNRDTYLR |
| ABBV-155 | | | |
| B7-H3 | LCDR2 | 185 | KVSNRFS |
| ABBV-155 | | | |
| B7-H3 | LCDR3 | 202 | SQSTHVPYT |
| ABBV-155 | | | |
| B7-H3 | HCDR1 | 203 | GYTFTNYVMH |
| DS-5573a | | | |
| B7-H3 | HCDR2 | 204 | YINPYNDDVKYNEKFKG |
| DS-5573a | | | |
| B7-H3 | HCDR3 | 205 | WGYYGSPLYYFDY |
| DS-5573a | | | |
| B7-H3 | LCDR1 | 206 | RASSRLIYMH |
| DS-5573a | | | |
| B7-H3 | LCDR2 | 207 | ATSNLAS |
| DS-5573a | | | |
| B7-H3 | LCDR3 | 208 | QQWNSNPPT |
| DS-5573a | | | |
| IgG | HCDR1 | 209 | GDSVSSNSAAWS |
| IgG | HCDR2 | 210 | RIYYRSKWYNDYAVSVKS |
| IgG | HCDR3 | 211 | LDHRYHEDTVYP GMDV |
| IgG | LCDR1 | 212 | SGDNLPAYTVT |
| IgG | LCDR2 | 213 | DDSDRPS |
| IgG | LCDR3 | 214 | ASWDPSSGVV |
| 9006 | HCDR1 | 349 | GYTFTNFR |
| 9006 | HCDR2 | 350 | INTYTGEP |
| 9006 | HCDR3 | 351 | ARKGIARAMDY |
| 9006 | LCDR1 | 352 | QSLLDSGNQKNY |
| 9006 | LCDR2 | 353 | GAS |
| 9006 | LCDR3 | 354 | QNDHSYPYT |
| 9338 | HCDR1 | 355 | GYTFTSYW |
| 9338 | HCDR2 | 356 | INPSSGHI |
| 9338 | HCDR3 | 357 | ARGRFAY |
| 9338 | LCDR1 | 358 | SSVSSGY |
| 9338 | LCDR2 | 359 | STS |
| 9338 | LCDR3 | 360 | HQWSSYPFT |
| 8902 | HCDR1 | 361 | GFSLTDYG |
| 8902 | HCDR2 | 362 | IWGGGST |
| 8902 | HCDR3 | 363 | AKTSYDGYYFDY |
| 8902 | LCDR1 | 364 | SSIN |
| 8902 | LCDR2 | 365 | DTS |
| 8902 | LCDR3 | 366 | QQWSFNPLT |

TABLE D4
Amino acid sequences of full-length mAb Ig chains

| mAb | IgG chain | SEQ ID NO | Amino acid sequence |
|---|---|---|---|
| J6M0 | Heavy chain | 57 | QVQLVQSGAEVKKPGSSVKVSCKASGGTF |
| | | | SNYWMHWVRQAPGQGLEWMGATYRGHSDT |
| | | | YYNQKFKGRVTITADKSTSTAYMELSSLR |
| | | | SEDTAVYYCARGAIYNGYDVLDNWGQGTL |
| | | | VTVSSASTKGPSVFPLAPSSKSTSGGTAA |
| | | | LGCLVKDYFPCPVTVSWNSGALTSGVHTF |
| | | | PAVLQSSGLYSLSSVVTVPSSSLGTQTYI |
| | | | CNVNHKPSNTKVDKRVEPKSCDKTHTCPP |
| | | | CPAPELLGGPSVFLFPPKPKDTLMISRTP |
| | | | EVTCVVVDVSHEDPEVKFNWYVDGVEVHN |
| | | | AKTKPREEQYNSTYRVVSVLTVLHQDWLN |
| | | | GKEYKCKVSNKALPAPIEKTISKAKGQPR |
| | | | EPQVYTLPPSREEMTKNQVSLTCLVKGFY |
| | | | PCDIAVEWESNGQPENNYKTTPPVLDSDG |
| | | | SFFLYSKLTVDKSRWQQGNVFSCSVMHEA |
| | | | LHNHYTQKSLSLSPGK |
| J6M0 | Light chain | 58 | DIQMTQSPSSLSASVGDRVTITCSASQDI |
| | | | SNYLNWYQQKPGKAPKLLIYYTSNLHSGV |
| | | | PSRFSGSGSGTDFTLTISSLQPEDFATYY |
| | | | CQQYRKLPWTFGQGTKLEIKRTVAAPSVE |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSENRGEC |
| MuMy9-6ch | Heavy chain | 59 | QVQLQQPGAEVVKPGASVKMSCKASGYTF |
| | | | TSYYIHWIKQTPGQGLEWVGVIYPGNDDI |
| | | | SYNQKFKGKATLTADKSSTTAYMQLSSLT |
| | | | SEDSAVYYCAREVRLRYFDVWGAGTTVTV |
| | | | SSASTKGPSVFPLAPSSKSTSGGTAALGC |
| | | | LVKDYFPCPVTVSWNSGALTSGVHTFPAV |
| | | | LQSSGLYSLSSVVTVPSSSLGTQTYICNV |
| | | | NHKPSNTKVDKRVEPKSCDKTHTCPPCPA |
| | | | PELLGGPSVFLFPPKPKDTLMISRTPEVT |
| | | | CVVVDVSHEDPEVKFNWYVDGVEVHNAKT |
| | | | KPREEQYNSTYRVVSVLTVLHQDWLNGKE |
| | | | YKCKVSNKALPAPIEKTISKAKGQPREPQ |
| | | | VYTLPPSREEMTKNQVSLTCLVKGFYPCD |
| | | | IAVEWESNGQPENNYKTTPPVLDSDGSFF |
| | | | LYSKLTVDKSRWQQGNVFSCSVMHEALHN |
| | | | HYTQKSLSLSPGK |
| MuMy9-6ch | Light chain | 60 | NIMLTQSPSSLAVSAGEKVTMSCKSSQSV |
| | | | FFSSSQKNYLAWYQQIPGQSPKLLIYWAS |
| | | | TRESGVPDRFTGSGSGTDFTLIISSVQSE |
| | | | DLAIYYCHQYLSSRTFGGGTKLEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSFNRGEC |
| gemtuzumab | Heavy chain | 61 | EVQLVQSGAEVKKPGSSVKVSCKASGYTI |
| | | | TDSNIHWVRQAPGQSLEWIGYIYPYNGGT |
| | | | DYNQKFKNRATLTVDNPTNTAYMELSSLR |
| | | | SEDTAFYYCVNGNPWLAYWGQGTLVTVSS |
| | | | ASTKGPSVFPLAPSSKSTSGGTAALGCLV |
| | | | KDYFPCPVTVSWNSGALTSGVHTFPAVLQ |
| | | | SSGLYSLSSVVTVPSSSLGTQTYICNVNH |
| | | | KPSNTKVDKRVEPKSCDKTHTCPPCPAPE |
| | | | LLGGPSVFLFPPKPKDTLMISRTPEVTCV |
| | | | VVDVSHEDPEVKFNWYVDGVEVHNAKTKP |
| | | | REEQYNSTYRVVSVLTVLHQDWLNGKEYK |
| | | | CKVSNKALPAPIEKTISKAKGQPREPQVY |
| | | | TLPPSREEMTKNQVSLTCLVKGFYPCDIA |
| | | | VEWESNGQPENNYKTTPPVLDSDGSFFLY |
| | | | SKLTVDKSRWQQGNVFSCSVMHEALHNHY |
| | | | TQKSLSLSPGK |
| gemtuzumab | Light chain | 62 | DIQLTQSPSTLSASVGDRVTITCRASESL |
| | | | DNYGIRFLTWFQQKPGKAPKLLMYAASNQ |
| | | | GSGVPSRFSGSGSGTEFTLTISSLQPDDE |
| | | | ATYYCQQTKEVPWSFGQGTKVEVKRTVAA |
| | | | PSVFIFPPSDEQLKSGTASVVCLLNNFYP |
| | | | REAKVQWKVDNALQSGNSQESVTEQDSKD |
| | | | STYSLSSTLTLSKADYEKHKVYACEVTHQ |
| | | | GLSSPVTKSFNRGEC |
| NOV169N31Q | Heavy chain | 63 | QVQLQQSGPGLVKPSQTLSLTCAISGDSV |
| | | | SSQSAAWNWIRQSPSRGLEWLGRIYYRSK |
| | | | WYNDYALSVKSRITINPDTSKNQFSLQLN |
| | | | SVTPEDTAVYYCARGEGYGREGFAIWGQG |
| | | | TLVTVSSASTKGPSVFPLAPSSKSTSGGT |
| | | | AALGCLVKDYFPCPVTVSWNSGALTSGVH |
| | | | TFPAVLQSSGLYSLSSVVTVPSSSLGTQT |
| | | | YICNVNHKPSNTKVDKRVEPKSCDKTHTC |
| | | | PPCPAPELLGGPSVFLFPPKPKDTLMISR |
| | | | TPEVTCVVVDVSHEDPEVKFNWYVDGVEV |
| | | | HNAKTKPREEQYNSTYRVVSVLTVLHQDW |
| | | | LNGKEYKCKVSNKALPAPIEKTISKAKGQ |
| | | | PREPQVYTLPPSREEMTKNQVSLTCLVKG |
| | | | FYPCDIAVEWESNGQPENNYKTTPPVLDS |
| | | | DGSFFLYSKLTVDKSRWQQGNVFSCSVMH |
| | | | EALHNHYTQKSLSLSPGK |
| NOV169N31Q | Light chain | 64 | DIQMTQSPSSLSASVGDRVTITCRASQTI |
| | | | SNTLAWYQQKPGKAPKLLIYAASNLQSGV |
| | | | PSRFSGSGSGTDFTLTISSLQPEDFATYY |
| | | | CQQYLSWFTFGQGTKVEIKRTVAAPSVFI |
| | | | FPPSDEQLKSGTASVVCLLNNFYPREAKV |
| | | | QWKVDNALQSGNSQESVTEQDSKDSTYSL |
| | | | SSTLTLSKADYEKHKVYACEVTHQGLSSP |
| | | | VTKSENRGEC |
| trastuzumab | Heavy chain | 65 | EVQLVESGGGLVQPGGSLRLSCAASGENI |
| | | | KDTYIHWVRQAPGKGLEWVARIYPTNGYT |
| | | | RYADSVKGRFTISADTSKNTAYLQMNSLR |
| | | | AEDTAVYYCSRWGGDGFYAMDYWGQGTLV |
| | | | TVSSASTKGPSVFPLAPSSKSTSGGTAAL |
| | | | GCLVKDYFPEPVTVSWNSGALTSGVHTFP |
| | | | AVLQSSGLYSLSSVVTVPSSSLGTQTYIC |
| | | | NVNHKPSNTKVDKKVEPPKSCDKTHTCPP |
| | | | CPAPELLGGPSVFLFPPKPKDTLMISRTP |
| | | | EVTCVVVDVSHEDPEVKFNWYVDGVEVHN |
| | | | AKTKPREEQYNSTYRVVSVLTVLHQDWLN |
| | | | GKEYKCKVSNKALPAPIEKTISKAKGQPR |
| | | | EPQVYTLPPSRDELTKNQVSLTCLVKGFY |
| | | | PSDIAVEWESNGQPENNYKTTPPVLDSDG |
| | | | SFFLYSKLTVDKSRWQQGNVFSCSVMHEA |
| | | | LHNHYTQKSLSLSPGK |
| trastuzumab | Light chain | 66 | DIQMTQSPSSLSASVGDRVTITCRASQDV |
| | | | NTAVAWYQQKPGKAPKLLIYSASFLYSGV |
| | | | PSRFSGSRSGTDFTLTISSLQPEDFATYY |
| | | | CQQHYTTPPTFGQGTKVEIKRTVAAPSVF |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSENRGEC |
| daratumumab | Heavy chain | 67 | EVQLLESGGGLVQPGGSLRLSCAVSGFTF |
| | | | NSFAMSWVRQAPGKGLEWVSAISGSGGGT |
| | | | YYADSVKGRFTISRDNSKNTLYLQMNSLR |
| | | | AEDTAVYFCAKDKILWFGEPVFDYWGQGT |
| | | | LVTVSSASTKGPSVFPLAPSSKSTSGGTA |
| | | | ALGCLVKDYFPCPVTVSWNSGALTSGVHT |
| | | | FPAVLQSSGLYSLSSVVTVPSSSLGTQTY |
| | | | ICNVNHKPSNTKVDKRVEPKSCDKTHTCP |
| | | | PCPAPELLGGPSVFLFPPKPKDTLMISRT |
| | | | PEVTCVVVDVSHEDPEVKFNWYVDGVEVH |
| | | | NAKTKPREEQYNSTYRVVSVLTVLHQDWL |
| | | | NGKEYKCKVSNKALPAPIEKTISKAKGQP |
| | | | REPQVYTLPPSREEMTKNQVSLTCLVKGF |
| | | | YPCDIAVEWESNGQPENNYKTTPPVLDSD |
| | | | GSFFLYSKLTVDKSRWQQGNVFSCSVMHE |
| | | | ALHNHYTQKSLSLSPGK |
| daratumumab | Light chain | 68 | EIVLTQSPATLSLSPGERATLSCRASQSV |
| | | | SSYLAWYQQKPGQAPRLLIYDASNRATGI |
| | | | PARFSGSGSGTDFTLTISSLEPEDFAVYY |
| | | | CQQRSNWPPTFGQGTKVEIKRTVAAPSVE |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSENRGEC |
| SGN-48A | Heavy chain | 69 | QVQLVQSGSELKKPGASVKVSCKASGYTF |
| | | | TDFGMNWVRQAPGQGLEWMGWINTFTGEP |
| | | | SYGNVFKGRFVFSLDTSVSTAYLQISSLK |
| | | | AEDTAVYYCARRHGNGNVFDSWGQGTLVT |
| | | | VSSASTKGPSVFPLAPSSKSTSGGTAALG |
| | | | CLVKDYFPCPVTVSWNSGALTSGVHTFPA |
| | | | VLQSSGLYSLSSVVTVPSSSLGTQTYICN |
| | | | VNHKPSNTKVDKRVEPKSCDKTHTCPPCP |
| | | | APELLGGPSVFLFPPKPKDTLMISRTPEV |
| | | | TCVVVDVSHEDPEVKFNWYVDGVEVHNAK |
| | | | TKPREEQYNSTYRVVSVLTVLHQDWLNGK |
| | | | EYKCKVSNKALPAPIEKTISKAKGQPREP |
| | | | QVYTLPPSREEMTKNQVSLTCLVKGFYPC |
| | | | DIAVEWESNGQPENNYKTTPPVLDSDGSF |
| | | | FLYSKLTVDKSRWQQGNVFSCSVMHEALH |
| | | | NHYTQKSLSLSPGK |
| SGN-48A | Light chain | 70 | EIVLTQSPDFQSVTPKEKVTITCRASQSI |
| | | | GSNIHWYQQKPDQSPKLLIKYTSESISGV |
| | | | PSRFSGSGSGTDFTLTINSLEAEDAATYY |
| | | | CQQSNSWPLTFGGGTKVEIKRTVAAPSVE |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| Polatuzimab | Heavy Chain | 88 | EVQLVESGGGLVQPGGSLRLSCAASGYTF |
| | | | SSYWIEWVRQAPGKGLEWIGEILPGGGDT |
| | | | NYNEIFKGRATFSADTSKNTAYLQMNSLR |
| | | | AEDTAVYYCTRRVPIRLDYWGQGTLVTVS |
| | | | SASTKGPSVFPLAPSSKSTSGGTAALGCL |
| | | | VKDYFPCPVTVSWNSGALTSGVHTFPAVL |
| | | | QSSGLYSLSSVVTVPSSSLGTQTYICNVN |
| | | | HKPSNTKVDKRVEPKSCDKTHTCPPCPAP |
| | | | ELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEY |
| | | | KCKVSNKALPAPIEKTISKAKGQPREPQV |
| | | | YTLPPSREEMTKNQVSLTCLVKGFYPCDI |
| | | | AVEWESNGQPENNYKTTPPVLDSDGSFFL |
| | | | YSKLTVDKSRWQQGNVFSCSVMHEALHNH |
| | | | YTQKSLSLSPGK |
| Polatuzimab | Light Chain | 89 | DIQLTQSPSSLSASVGDRVTITCKASQSV |
| | | | DYEGDSFLNWYQQKPGKAPKLLIYAASNL |
| | | | ESGVPSRFSGSGSGTDFTLTISSLQPEDF |
| | | | ATYYCQQSNEDPLTFGQGTKVEIKRTVAA |
| | | | PSVFIFPPSDEQLKSGTASVVCLLNNFYP |
| | | | REAKVQWKVDNALQSGNSQESVTEQDSKD |
| | | | STYSLSSTLTLSKADYEKHKVYACEVTHQ |
| | | | GLSSPVTKSFNRGEC |
| VHmil x VK1aNQ | Heavy Chain | 118 | QVQLQQSGSELKKPGASVKVSCKASGYTF |
| | | | TNYGVNWIKQAPGQGLQWMGWINPNTGEP |
| | | | TFDDDFKGRFAFSLDTSVSTAYLQISSLK |
| | | | ADDTAVYFCSRSRGKNEAWFAYWGQGTLV |
| | | | TVSSASTKGPSVFPLAPSSKSTSGGTAAL |
| | | | GCLVKDYFPCPVTVSWNSGALTSGVHTFP |
| | | | AVLQSSGLYSLSSVVTVPSSSLGTQTYIC |
| | | | NVNHKPSNTKVDKRVEPKSCDKTHTCPPC |
| | | | PAPELLGGPSVFLFPPKPKDTLMISRTPE |
| | | | VTCVVVDVSHEDPEVKFNWYVDGVEVHNA |
| | | | KTKPREEQYNSTYRVVSVLTVLHQDWLNG |
| | | | KEYKCKVSNKALPAPIEKTISKAKGQPRE |
| | | | PQVYTLPPSREEMTKNQVSLTCLVKGFYP |
| | | | CDIAVEWESNGQPENNYKTTPPVLDSDGS |
| | | | FFLYSKLTVDKSRWQQGNVFSCSVMHEAL |
| | | | HNHYTQKSLSLSPGK |
| | Light Chain | 216 | DIVMTQTPLSLPVTPGEPASISCRSSQSL |
| | | | VHRNQNTYLHWYLQKPGQSPQLLIYTVSN |
| | | | RFSGVPDRFSGSGSGTDFTLKISRVEAED |
| | | | VGVYFCSQSSHVPPTFGQGTKLEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSFNRGEC |
| SEZ6 | Heavy chain | 217 | QVQLVQSGAEVKKPGASVKVSCKASGYTF |
| | | | TSYWINWVRQAPGQGLEWIGNIFPDTTTT |
| | | | NYNEKFKGRVTLTRDTSISTAYMELSRLR |
| | | | SDDTAVYYCAREYYDGTYDAMDYWGQGTL |
| | | | VTVSSASTKGPSVFPLAPSSKSTSGGTAA |
| | | | LGCLVKDYFPCPVTVSWNSGALTSGVHTF |
| | | | PAVLQSSGLYSLSSVVTVPSSSLGTQTYI |
| | | | CNVNHKPSNTKVDKRVEPKSCDKTHTCPP |
| | | | CPAPELLGGPSVFLFPPKPKDTLMISRTP |
| | | | EVTCVVVDVSHEDPEVKFNWYVDGVEVHN |
| | | | AKTKPREEQYNSTYRVVSVLIVLHQDWLN |
| | | | GKEYKCKVSNKALPAPIEKTISKAKGQPR |
| | | | EPQVYTLPPSREEMTKNQVSLTCLVKGFY |
| | | | PCDIAVEWESNGQPENNYKTTPPVLDSDG |
| | | | SFFLYSKLTVDKSRWQQGNVFSCSVMHEA |
| | | | LHNHYTQKSLSLSPGK |
| SEZ6 | Light chain | 218 | AIQMTQSPSSLSASVGDRVTITCKASQSV |
| | | | NNDVAWYQQKPGKAPKLLIYYASNRYTGV |
| | | | PSRFSGSGSGTDFTLTISSLQPEDFATYF |
| | | | CQQDYSSPRTFGQGTKLEIKRTVAAPSVE |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| CD56 | Heavy chain | 219 | QVQLVESGGGVVQPGRSLRLSCAASGFTF |
| (Lorovtuzumab) | | | SSFGMHWVRQAPGKGLEWVAYISSGSFTI |
| | | | YYADSVKGRFTISRDNSKNTLYLQMNSLR |
| | | | AEDTAVYYCARMRKGYAMDYWGQGTLVTV |
| | | | SSASTKGPSVFPLAPSSKSTSGGTAALGC |
| | | | LVKDYFPCPVTVSWNSGALTSGVHTFPAV |
| | | | LQSSGLYSLSSVVTVPSSSLGTQTYICNV |
| | | | NHKPSNTKVDKRVEPKSCDKTHTCPPCPA |
| | | | PELLGGPSVFLFPPKPKDTLMISRTPEVT |
| | | | CVVVDVSHEDPEVKFNWYVDGVEVHNAKT |
| | | | KPREEQYNSTYRVVSVLTVLHQDWLNGKE |
| | | | YKCKVSNKALPAPIEKTISKAKGQPREPQ |
| | | | VYTLPPSREEMTKNQVSLTCLVKGFYPCD |
| | | | IAVEWESNGQPENNYKTTPPVLDSDGSFF |
| | | | LYSKLTVDKSRWQQGNVFSCSVMHEALHN |
| | | | HYTQKSLSLSPGK |
| CD56 | Light chain | 220 | DVVMTQSPLSLPVTLGQPASISCRSSQII |
| | | | IHSDGNTYLEWFQQRPGQSPRRLIYKVSN |
| | | | RFSGVPDRFSGSGSGTDFTLKISRVEAED |
| | | | VGVYYCFQGSHVPHTFGQGTKVEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSFNRGEC |
| DLL3 | Heavy chain | 221 | QVQLVQSGAEVKKPGASVKVSCKASGYTF |
| (Rovalpituzumab) | | | TNYGMNWVRQAPGQGLEWMGWINTYTGEP |
| | | | TYADDFKGRVTMTTDTSTSTAYMELRSLR |
| | | | SDDTAVYYCARIGDSSPSDYWGQGTLVTV |
| | | | SSASTKGPSVFPLAPSSKSTSGGTAALGC |
| | | | LVKDYFPCPVTVSWNSGALTSGVHTFPAV |
| | | | LQSSGLYSLSSVVTVPSSSLGTQTYICNV |
| | | | NHKPSNTKVDKRVEPKSCDKTHTCPPCPA |
| | | | PELLGGPSVFLFPPKPKDTLMISRTPEVT |
| | | | CVVVDVSHEDPEVKFNWYVDGVEVHNAKT |
| | | | KPREEQYNSTYRVVSVLTVLHQDWLNGKE |
| | | | YKCKVSNKALPAPIEKTISKAKGQPREPQ |
| | | | VYTLPPSREEMTKNQVSLTCLVKGFYPCD |
| | | | IAVEWESNGQPENNYKTTPPVLDSDGSFF |
| | | | LYSKLTVDKSRWQQGNVFSCSVMHEALHN |
| | | | HYTQKSLSLSPGK |
| DLL3 | Light chain | 222 | EIVMTQSPATLSVSPGERATLSCKASQSV |
| | | | SNDVVWYQQKPGQAPRLLIYYASNRYTGI |
| | | | PARFSGSGSGTEFTLTISSLQSEDFAVYY |
| | | | CQQDYTSPWTFGQGTKLEIKRTVAAPSVE |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| DLK1 | Heavy chain | 223 | EVQLQQSGAELVKPGASVKLSCTASGFNI |
| (DI-2-14) | | | RDTYIHWVKQRPEQGLEWIGRIDPPNGNL |
| | | | KYDPKFQGKATITADTSSNTAYLQFSSLT |
| | | | SEDTAVYYCARSDGYSFAYWGQGTLVTVS |
| | | | SASTKGPSVFPLAPSSKSTSGGTAALGCL |
| | | | VKDYFPCPVTVSWNSGALTSGVHTFPAVL |
| | | | QSSGLYSLSSVVTVPSSSLGTQTYICNVN |
| | | | HKPSNTKVDKRVEPKSCDKTHTCPPCPAP |
| | | | ELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEY |
| | | | KCKVSNKALPAPIEKTISKAKGQPREPQV |
| | | | YTLPPSREEMTKNQVSLTCLVKGFYPCDI |
| | | | AVEWESNGQPENNYKTTPPVLDSDGSFFL |
| | | | YSKLTVDKSRWQQGNVFSCSVMHEALHNH |
| | | | YTQKSLSLSPGK |
| DLK1 | Light chain | 224 | DIVMTQAAPSVPVTPGESVSISCRSSKSL |
| | | | LHSNGNTYLYWFLQRPGQSPQLLIYRMSN |
| | | | LASGVPDRFSGSGSGTAFTLRISRVEAED |
| | | | VGVYYCMQHVEYPFTFGSGTKLEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSENRGEC |
| B7-H3 | Heavy chain | 225 | EVQLVQSGAEVKKPGSSVKVSCKASGYTF |
| ABBV-155 | | | SSYWMHWVRQAPGQGLEWIGLIHPESGST |
| | | | NYNEMFKNRATLTVDRSTSTAYMELSSLR |
| | | | SEDTAVYYCAGGGRLYFDYWGQGTTVTVS |
| | | | SASTKGPSVFPLAPSSKSTSGGTAALGCL |
| | | | VKDYFPCPVTVSWNSGALTSGVHTFPAVL |
| | | | QSSGLYSLSSVVTVPSSSLGTQTYICNVN |
| | | | HKPSNTKVDKRVEPKSCDKTHTCPPCPAP |
| | | | ELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEY |
| | | | KCKVSNKALPAPIEKTISKAKGQPREPQV |
| | | | YTLPPSREEMTKNQVSLTCLVKGFYPCDI |
| | | | AVEWESNGQPENNYKTTPPVLDSDGSFFL |
| | | | YSKLTVDKSRWQQGNVFSCSVMHEALHNH |
| | | | YTQKSLSLSPGK |
| B7-H3 | Light chain | 226 | DIVMTQSPLSLPVTPGEPASISCRSSQSL |
| ABBV-155 | | | VHSNRDTYLRWYLQKPGQSPQLLIYKVSN |
| | | | RFSGVPDRFSGSGSGTDFTLKISRVEAED |
| | | | VGVYYCSQSTHVPYTFGGGTKVEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSENRGEC |
| B7-H3 | Heavy Chain | 227 | QVQLVQSGAEVKKPGSSVKVSCKASGYTF |
| DS-5573a | | | TNYVMHWVRQAPGQGLEWMGYINPYNDDV |
| | | | KYNEKFKGRVTITADESTSTAYMELSSLR |
| | | | SEDTAVYYCARWGYYGSPLYYFDYWGQGT |
| | | | LVTVSSASTKGPSVFPLAPSSKSTSGGTA |
| | | | ALGCLVKDYFPCPVTVSWNSGALTSGVHT |
| | | | FPAVLQSSGLYSLSSVVTVPSSSLGTQTY |
| | | | ICNVNHKPSNTKVDKRVEPKSCDKTHTCP |
| | | | PCPAPELLGGPSVFLFPPKPKDTLMISRT |
| | | | PEVTCVVVDVSHEDPEVKFNWYVDGVEVH |
| | | | NAKTKPREEQYNSTYRVVSVLTVLHQDWL |
| | | | NGKEYKCKVSNKALPAPIEKTISKAKGQP |
| | | | REPQVYTLPPSREEMTKNQVSLTCLVKGF |
| | | | YPCDIAVEWESNGQPENNYKTTPPVLDSD |
| | | | GSFFLYSKLTVDKSRWQQGNVFSCSVMHE |
| | | | ALHNHYTQKSLSLSPGK |
| B7-H3 | Light Chain | 228 | EIVLTQSPATLSLSPGERATLSCRASSRL |
| DS-5573a | | | IYMHWYQQKPGQAPRPLIYATSNLASGIP |
| | | | ARFSGSGSGTDFTLTISSLEPEDFAVYYC |
| | | | QQWNSNPPTFGQGTKVEIKRTVAAPSVFI |
| | | | FPPSDEQLKSGTASVVCLLNNFYPREAKV |
| | | | QWKVDNALQSGNSQESVTEQDSKDSTYSL |
| | | | SSTLTLSKADYEKHKVYACEVTHQGLSSP |
| | | | VTKSFNRGEC |
| IgG | Heavy Chain | 229 | QVQLQQSGPGLVKPSQTLSLTCAISGDSV |
| | | | SSNSAAWSWIRQSPGRGLEWLGRIYYRSK |
| | | | WYNDYAVSVKSRITINPDTSKNQFSLQLN |
| | | | SVTPEDTAVYYCARLDHRYHEDTVYPGMD |
| | | | VWGQGTLVTVSSASTKGPSVFPLAPSSKS |
| | | | TSGGTAALGCLVKDYFPEPVTVSWNSGAL |
| | | | TSGVHTFPAVLQSSGLYSLSSVVTVPSSS |
| | | | LGTQTYICNVNHKPSNTKVDKRVEPKSCD |
| | | | KTHTCPPCPAPELLGGPSVFLFPPKPKDT |
| | | | LMISRTPEVTCVVVDVSHEDPEVKENWYV |
| | | | DGVEVHNAKTKPREEQYNSTYRVVSVLTV |
| | | | LHQDWLNGKEYKCKVSNKALPAPIEKTIS |
| | | | KAKGQPREPQVYTLPPSREEMTKNQVSLT |
| | | | CLVKGFYPSDIAVEWESNGQPENNYKTTP |
| | | | PVLDSDGSFFLYSKLTVDKSRWQQGNVFS |
| | | | CSVMHEALHNHYTQKSLSLSPG |
| IgG | Light Chain | 230 | DIELTQPPSVSVAPGQTARISCSGDNLPA |
| | | | YTVTWYQQKPGQAPVLVIYDDSDRPSGIP |
| | | | ERFSGSNSGNTATLTISGTQAEDEADYYC |
| | | | ASWDPSSGVVFGGGTKLTVLGQPKAAPSV |
| | | | TLFPPSSEELQANKATLVCLISDFYPGAV |
| | | | TVAWKADSSPVKAGVETTTPSKQSNNKYA |
| | | | ASSYLSLTPEQWKSHRSYSCQVTHEGSTV |
| | | | EKTVAPTECS |
| 9006 IgG1 | Heavy chain | 367 | QVQLVQSGSELKKPGASVKVSCKASGYTF |
| (as described in | | | TNFRMNWVKQAPGQGLKWMGWINTYTGEP |
| WO2016/042412) | | | TYVDDLKGRFVFSLDTSVSTAYLQISSLK |
| | | | AEDTAVYYCARKGIARAMDYWGQGTTVTV |
| | | | SSASTKGPSVFPLAPSSKSTSGGTAALGC |
| | | | LVKDYFPEPVTVSWNSGALTSGVHTFPAV |
| | | | LQSSGLYSLSSVVTVPSSSLGTQTYICNV |
| | | | NHKPSNTKVDKRVEPKSCDKTHTCPPCPA |
| | | | PELLGGPSVFLFPPKPKDTLMISRTPEVT |
| | | | CVVVDVSHEDPEVKFNWYVDGVEVHNAKT |
| | | | KPREEQYNSTYRVVSVLTVLHQDWLNGKE |
| | | | YKCKVSNKALPAPIEKTISKAKGQPREPQ |
| | | | VYTLPPSREEMTKNQVSLTCLVKGFYPSD |
| | | | IAVEWESNGQPENNYKTTPPVLDSDGSFF |
| | | | LYSKLTVDKSRWQQGNVFSCSVMHEALHN |
| | | | HYTQKSLSLSPG |
| 9006 IgG1 | Light chain | 368 | DIVMTQSPDSLAVSLGERATINCKSSQSL |
| (as described in | | | LDSGNQKNYLAWYQQKPGQPPKLLIFGAS |
| WO2016/042412) | | | TRESGVPDRFSGSGSGTDFTLTISSLQAE |
| | | | DVAVYYCQNDHSYPYTFGQGTKLEIKRTV |
| | | | AAPSVFIFPPSDEQLKSGTASVVCLLNNF |
| | | | YPREAKVQWKVDNALQSGNSQESVTEQDS |
| | | | KDSTYSLSSTLTLSKADYEKHKVYACEVT |
| | | | HQGLSSPVTKSFNRGEC |
| 9338 IgG1 | Heavy chain | 369 | QVQLVQSGAEVKKPGSSVKVSCKASGYTF |
| (as described in | | | TSYWMHWVRQAPGQGLEWMGYINPSSGHI |
| WO2016/042412) | | | ENNQKFKDRVTITADKSTSTAYMELSSLR |
| | | | SEDTAVYYCARGRFAYWGQGTLVTVSSAS |
| | | | TKGPSVFPLAPSSKSTSGGTAALGCLVKD |
| | | | YFPEPVTVSWNSGALTSGVHTFPAVLQSS |
| | | | GLYSLSSVVTVPSSSLGTQTYICNVNHKP |
| | | | SNTKVDKRVEPKSCDKTHTCPPCPAPELL |
| | | | GGPSVFLFPPKPKDTLMISRTPEVTCVVV |
| | | | DVSHEDPEVKFNWYVDGVEVHNAKTKPRE |
| | | | EQYNSTYRVVSVLTVLHQDWLNGKEYKCK |
| | | | VSNKALPAPIEKTISKAKGQPREPQVYTL |
| | | | PPSREEMTKNQVSLTCLVKGFYPSDIAVE |
| | | | WESNGQPENNYKTTPPVLDSDGSFFLYSK |
| | | | LTVDKSRWQQGNVFSCSVMHEALHNHYTQ |
| | | | KSLSLSPG |
| 9338 IgG1 | Light chain | 370 | EIVLTQSPATLSLSPGERATLSCSASSSV |
| (as described in | | | SSGYLYWYQQKPGQAPRLLIYSTSNLASG |
| WO2016/042412) | | | IPARFSGSGSGTDFTLTISSLEPEDFAVY |
| | | | YCHQWSSYPFTFGSGTKLEIKRTVAAPSV |
| | | | FIFPPSDEQLKSGTASVVCLLNNFYPREA |
| | | | KVQWKVDNALQSGNSQESVTEQDSKDSTY |
| | | | SLSSTLTLSKADYEKHKVYACEVTHQGLS |
| | | | SPVTKSFNRGEC |
| 8902 IgG1 | Heavy chain | 371 | QVQLQESGPGLVKPSQTLSLTCTVSGFSL |
| | | | TDYGVSWIRQPPGKGLEWIGVIWGGGSTY |
| | | | HNSALKSRVTISVDTSKNQFSLKLSSVTA |
| | | | ADTAVYYCAKTSYDGYYFDYWGQGTLVTV |
| | | | SSASTKGPSVFPLAPSSKSTSGGTAALGC |
| | | | LVKDYFPEPVTVSWNSGALTSGVHTFPAV |
| | | | LQSSGLYSLSSVVTVPSSSLGTQTYICNV |
| | | | NHKPSNTKVDKRVEPKSCDKTHTCPPCPA |
| | | | PELLGGPSVFLFPPKPKDTLMISRTPEVT |
| | | | CVVVDVSHEDPEVKFNWYVDGVEVHNAKT |
| | | | KPREEQYNSTYRVVSVLTVLHQDWLNGKE |
| | | | YKCKVSNKALPAPIEKTISKAKGQPREPQ |
| | | | VYTLPPSREEMTKNQVSLTCLVKGFYPSD |
| | | | IAVEWESNGQPENNYKTTPPVLDSDGSFF |
| | | | LYSKLTVDKSRWQQGNVFSCSVMHEALHN |
| | | | HYTQKSLSLSPGK |
| 8902 IgG1 | Light chain | 372 | EIVLTQSPATLSLSPGERATLSCSASSSI |
| | | | NNMHWYQQKPGQAPRLLIYDTSKLASGIP |
| | | | ARFSGSGSGTDFTLTISSLEPEDFAVYYC |
| | | | QQWSENPLTFGQGTKLEIKRTVAAPSVFI |
| | | | FPPSDEQLKSGTASVVCLLNNFYPREAKV |
| | | | QWKVDNALQSGNSQESVTEQDSKDSTYSL |
| | | | SSTLTLSKADYEKHKVYACEVTHQGLSSP |
| | | | VTKSENRGEC |
| 9006 IgG2 | Heavy chain | 373 | QVQLVQSGSELKKPGASVKVSCKASGYTF |
| | | | TNFRMNWVKQAPGQGLKWMGWINTYTGEP |
| | | | TYVDDLKGRFVFSLDTSVSTAYLQISSLK |
| | | | AEDTAVYYCARKGIARAMDYWGQGTTVTV |
| | | | SSASTKGPSVEPLAPCSRSTSESTAALGC |
| | | | LVKDYFPEPVTVSWNSGALISGVHTFPAV |
| | | | LQSSGLYSLSSVVTVPSSNFGTQTYTCNV |
| | | | DHKPSNTKVDKTVERKCCVECPPCPAPPV |
| | | | AGPSVFLFPPKPKDTLMISRTPEVTCVVV |
| | | | DVSHEDPEVQFNWYVDGVEVHNAKTKPRE |
| | | | EQFNSIFRVVSVLIVVHQDWINGKEYKCK |
| | | | VSNKGLPAPIEKTISKTKGQPREPQVYTL |
| | | | PPSREEMTKNQVSLTCLVKGFYPSDISVE |
| | | | WESNGQPENNYKTTPPMLDSDGSFFLYSK |
| | | | LTVDKSRWQQGNVFSCSVMHEALHNHYTQ |
| | | | KSLSLSPGK |
| 9006 IgG2 | Light chain | 374 | DIVMTQSPDSLAVSLGERATINCKSSQSL |
| | | | LDSGNQKNYLAWYQQKPGQPPKLLIFGAS |
| | | | TRESGVPDRFSGSGSGTDFTLTISSLQAE |
| | | | DVAVYYCQNDHSYPTFGQGTKLEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSENRGEC |
| 9338 IgG2 | Heavy chain | 375 | QVQLVQSGAEVKKPGSSVKVSCKASGYTF |
| | | | TSYWMHWVRQAPGQGLEWMGYINPSSGHI |
| | | | ENNQKFKDRVTITADKSTSTAYMELSSLR |
| | | | SEDTAVYYCARGRFAYWGQGTLVTVSSAS |
| | | | TKGPSVFPLAPCSRSTSESTAALGCLVKD |
| | | | YFPEPVTVSWNSGALTSGVHTFPAVLQSS |
| | | | GLYSLSSVVTVPSSNFGTQTYTCNVDHKP |
| | | | SNTKVDKTVERKCCVECPPCPAPPVAGPS |
| | | | VFLFPPKPKDTLMISRTPEVTCVVVDVSH |
| | | | EDPEVQFNWYVDGVEVHNAKTKPREEQFN |
| | | | STFRVVSVLTVVHQDWLNGKEYKCKVSNK |
| | | | GLPAPIEKTISKTKGQPREPQVYTLPPSR |
| | | | EEMTKNQVSLTCLVKGFYPSDISVEWESN |
| | | | GQPENNYKTTPPMLDSDGSFFLYSKLTVD |
| | | | KSRWQQGNVFSCSVMHEALHNHYTQKSLS |
| | | | LSPGK |
| 9338 IgG2 | Light chain | 370 | EIVLTQSPATLSLSPGERATLSCSASSSV |
| | | | SSGYLYWYQQKPGQAPRLLIYSTSNLASG |
| | | | IPARESGSGSGTDFTLTISSLEPEDFAVY |
| | | | YCHQWSSYPFTFGSGTKLEIKRTVAAPSV |
| | | | FIFPPSDEQLKSGTASVVCLLNNFYPREA |
| | | | KVQWKVDNALQSGNSQESVTEQDSKDSTY |
| | | | SLSSTLTLSKADYEKHKVYACEVTHQGLS |
| | | | SPVTKSENRGEC |
| 8902 IgG2 | Heavy chain | 376 | QVQLQESGPGLVKPSQTLSLTCTVSGFSL |
| | | | TDYGVSWIRQPPGKGLEWIGVIWGGGSTY |
| | | | HNSALKSRVTISVDTSKNQFSLKLSSVTA |
| | | | ADTAVYYCAKTSYDGYYFDYWGQGTLVTV |
| | | | SSASTKGPSVFPLAPCSRSTSESTAALGC |
| | | | LVKDYFPEPVTVSWNSGALTSGVHTFPAV |
| | | | LQSSGLYSLSSVVTVPSSNFGTQTYTCNV |
| | | | DHKPSNTKVDKTVERKCCVECPPCPAPPV |
| | | | AGPSVFLFPPKPKDTLMISRTPEVTCVVV |
| | | | DVSHEDPEVQFNWYVDGVEVHNAKTKPRE |
| | | | EQFNSTFRVVSVLTVVHQDWLNGKEYKCK |
| | | | VSNKGLPAPIEKTISKTKGQPREPQVYTL |
| | | | PPSREEMTKNQVSLTCLVKGFYPSDISVE |
| | | | WESNGQPENNYKTTPPMLDSDGSFFLYSK |
| | | | LTVDKSRWQQGNVFSCSVMHEALHNHYTQ |
| | | | KSLSLSPGK |
| 8902 IgG2 | Light chain | 372 | EIVLTQSPATLSLSPGERATLSCSASSSI |
| | | | NNMHWYQQKPGQAPRLLIYDTSKLASGIP |
| | | | ARFSGSGSGTDFTLTISSLEPEDFAVYYC |
| | | | QQWSFNPLTFGQGTKLEIKRTVAAPSVFI |
| | | | FPPSDEQLKSGTASVVCLLNNFYPREAKV |
| | | | QWKVDNALQSGNSQESVTEQDSKDSTYSL |
| | | | SSTLTLSKADYEKHKVYACEVTHQGLSSP |
| | | | VTKSENRGEC |

TABLE D5
Amino acid sequences of full-length mAb Ig chains

| Antibody | | Heavy Chain | Light Chain |
|---|---|---|---|
| Target/ | | (CysMab) | (CysMab) |
| Code | Antibody Name | Sequence | Sequence |
| EGFR1 | Cetuximab | QVQLKQSGPGLVOPSQSL | DILLTQSPVILSVSPGERVSFS |
| | | SITCTVSGFSLTNYGVHW | CRASQSIGTNIHWYQQRINGSP |
| | | VRQSPGKGLEWLGVIWSG | RLLIKYASESISGIPSRFSGSG |
| | | GNTDYNTPFTSRLSINKD | SGTDFTLSINSVESEDIADYYC |
| | | NSKSQVFFKMNSLOSNDT | QQNNNWPTTFGAGTKLELKRTV |
| | | AIYYCARALTYYDYEFAY | AAPSVFIFPPSDEQLKSGTASV |
| | | WGQGTLVTVSAASTKGPS | VCLLNNFYPREAKVQWKVDNAL |
| | | VFPLAPSSKSTSGGTAAL | QSGNSQESVTEQDSKDSTYSLS |
| | | GCLVKDYFPCPVTVSWNS | STLTLSKADYEKHKVYACEVTH |
| | | GALTSGVHTFPAVLQSSG | QGLSSPVTKSENRGEC (SEQ |
| | | LYSLSSVVTVPSSSLGTQ | ID NO: 93) |
| | | TYICNVNHKPSNTKVDKR | |
| | | VEPKSCDKTHTCPPCPAP | |
| | | ELLGGPSVFLFPPKPKDT | |
| | | LMISRTPEVTCVVVDVSH | |
| | | EDPEVKFNWYVDGVEVHN | |
| | | AKTKPREEQYNSTYRVVS | |
| | | VLTVLHQDWLNGKEYKCK | |
| | | VSNKALPAPIEKTISKAK | |
| | | GQPREPQVYTLPPSREEM | |
| | | TKNQVSLTCLVKGFYPCD | |
| | | IAVEWESNGQPENNYKTT | |
| | | PPVLDSDGSFFLYSKLTV | |
| | | DKSRWQQGNVFSCSVMHE | |
| | | ALHNHYTQKSLSLSPGK | |
| | | (SEQ ID NO: 92) | |
| TFRC | CD71 (CX-2029) | QVQLVQSGAEVKKPGASV | DIVLTQSPLSLAVSLGQPAIIS |
| | | KMSCKASGYTFTSYWMHW | CKASQSVSFAGTSLMHWYHQKP |
| | | VRQAPGQGLEWIGAIYPG | GQQPRLLIYRASNLEAGVPDRF |
| | | NSETGYAQKFQGRATLTA | SGSGSKTDFTLTISPVEAEDAA |
| | | DTSTSTAYMELSSLRSED | TYYCQQSREYPYTFGGGTKLEI |
| | | TAVYYCTRENWDPGFAFW | KRTVAAPSVFIFPPSDEQLKSG |
| | | GQGTLITVSSASTKGPSV | TASVVCLLNNFYPREAKVQWKV |
| | | FPLAPSSKSTSGGTAALG | DNALQSGNSQESVTEQDSKDST |
| | | CLVKDYFPCPVTVSWNSG | YSLSSTLTLSKADYEKHKVYAC |
| | | ALTSGVHTFPAVLQSSGL | EVTHQGLSSPVTKSENRGEC |
| | | YSLSSVVTVPSSSLGTQT | (SEQ ID NO: 95) |
| | | YICNVNHKPSNTKVDKRV | |
| | | EPKSCDKTHTCPPCPAPE | |
| | | LLGGPSVFLFPPKPKDTL | |
| | | MISRTPEVTCVVVDVSHE | |
| | | DPEVKFNWYVDGVEVHNA | |
| | | KTKPREEQYNSTYRVVSV | |
| | | LTVLHQDWLNGKEYKCKV | |
| | | SNKALPAPIEKTISKAKG | |
| | | QPREPQVYTLPPSREEMT | |
| | | KNQVSLTCLVKGFYPCDI | |
| | | AVEWESNGQPENNYKTTP | |
| | | PVLDSDGSFFLYSKLTVD | |
| | | KSRWQQGNVFSCSVMHEA | |
| | | LHNHYTQKSLSLSPGK | |
| | | (SEQ ID NO: 94) | |
| EPCAM | Oportuzumab* | EVQLVQSGPGLVQPGGSV | DIQMTQSPSSLSASVGDRVTIT |
| | *Variable heavy | RISCAASGYTFTNYGMNW | CRSTKSLLHSNGITYLYWYQQK |
| | chain and variable | VKQAPGKGLEWMGWINTY | PGKAPKLLIYQMSNLASGVPSR |
| | light chain sequence | TGESTYADSFKGRFTFSL | FSSSGSGTDFTLTISSLQPEDF |
| | of oportuzumab | DTSASAAYLQINSLRAED | ATYYCAQNLEIPRTFGQGTKVE |
| | monatox were | TAVYYCARFAIKGDYWGQ | LKRTVAAPSVFIFPPSDEQLKS |
| | combined with | GTLLTVSSASTKGPSVFP | GTASVVCLLNNFYPREAKVOWK |
| | human heavy chain | LAPSSKSTSGGTAALGCL | VDNALQSGNSQESVTEQDSKDS |
| | constant region and | VKDYFPCPVTVSWNSGAL | TYSLSSTLTLSKADYEKHKVYA |
| | light chain constant | TSGVHTFPAVLQSSGLYS | CEVTHQGLSSPVTKSFNRGEC |
| | region to produce | LSSVVTVPSSSLGTQTYI | (SEQ ID NO: 97) |
| | full IgG antibody | CNVNHKPSNTKVDKRVEP | |
| | sequence | KSCDKTHTCPPCPAPELL | |
| | | GGPSVFLFPPKPKDTLMI | |
| | | SRTPEVTCVVVDVSHEDP | |
| | | EVKFNWYVDGVEVHNAKT | |
| | | KPREEQYNSTYRVVSVLT | |
| | | VLHQDWLNGKEYKCKVSN | |
| | | KALPAPIEKTISKAKGQP | |
| | | REPQVYTLPPSREEMTKN | |
| | | QVSLTCLVKGFYPCDIAV | |
| | | EWESNGQPENNYKTTPPV | |
| | | LDSDGSFFLYSKLTVDKS | |
| | | RWQQGNVFSCSVMHEALH | |
| | | NHYTQKSLSLSPGK | |
| | | (SEQ ID NO: 96) | |
| FOLR1 | Mirvetuximab | QVQLVQSGAEVVKPGASV | DIVLTQSPLSLAVSLGQPAIIS |
| | | KISCKASGYTFTGYFMNW | CKASQSVSFAGTSLMHWYHQKP |
| | | VKQSPGQSLEWIGRIHPY | GQQPRLLIYRASNLEAGVPDRF |
| | | DGDTFYNQKFQGKATLTV | SGSGSKTDFTLTISPVEAEDAA |
| | | DKSSNTAHMELLSLTSED | TYYCQQSREYPYTFGGGTKLEI |
| | | FAVYYCTRYDGSRAMDYW | KRTVAAPSVFIFPPSDEQLKSG |
| | | GQGTTVTVSSASTKGPSV | TASVVCLLNNFYPREAKVQWKV |
| | | FPLAPSSKSTSGGTAALG | DNALQSGNSQESVTEQDSKDST |
| | | CLVKDYFPCPVTVSWNSG | YSLSSTLTLSKADYEKHKVYAC |
| | | ALTSGVHTFPAVLQSSGL | EVTHQGLSSPVTKSENRGEC |
| | | YSLSSVVTVPSSSLGTQT | (SEQ ID NO: 99) |
| | | YICNVNHKPSNTKVDKRV | |
| | | EPKSCDKTHTCPPCPAPE | |
| | | LLGGPSVFLFPPKPKDTL | |
| | | MISRTPEVTCVVVDVSHE | |
| | | DPEVKFNWYVDGVEVHNA | |
| | | KTKPREEQYNSTYRVVSV | |
| | | LTVLHQDWLNGKEYKCKV | |
| | | SNKALPAPIEKTISKAKG | |
| | | QPREPQVYTLPPSREEMT | |
| | | KNQVSLTCLVKGFYPCDI | |
| | | AVEWESNGQPENNYKTTP | |
| | | PVLDSDGSFFLYSKLTVD | |
| | | KSRWQQGNVFSCSVMHEA | |
| | | LHNHYTQKSLSLSPGK | |
| | | (SEQ ID NO: 98) | |
| ENPP3 | ENPP3 | QVQLQESGPGLVKPSQTL | EIVLTQSPDFQSVTPKEKVTIT |
| | (AGS16-7.8) | SLTCTVSGGSISSGGYYW | CRASQSIGISLHWYQQKPDQSP |
| | | SWIRQHPGKGLEWIGIIY | KLLIKYASQSFSGVPSRFSGSG |
| | | YSGSTYYNPSLKSRVTIS | SGTDFTLTINSLEAEDAATYYC |
| | | VDTSKNQFSLKLNSVTAA | HQSRSFPWTFGQGTKVEIKRTV |
| | | DTAVFYCARVAIVTTIPG | AAPSVFIFPPSDEQLKSGTASV |
| | | GMDVWGQGTTVTVSSAST | VCLLNNFYPREAKVQWKVDNAL |
| | | KGPSVFPLAPSSKSTSGG | QSGNSQESVTEQDSKDSTYSLS |
| | | TAALGCLVKDYFPCPVTV | STLTLSKADYEKHKVYACEVTH |
| | | SWNSGALTSGVHTFPAVL | QGLSSPVTKSENRGEC |
| | | QSSGLYSLSSVVTVPSSS | (SEQ ID NO: 101) |
| | | LGTQTYICNVNHKPSNTK | |
| | | VDKRVEPKSCDKTHTCPP | |
| | | CPAPELLGGPSVFLFPPK | |
| | | PKDTLMISRTPEVTCVVV | |
| | | DVSHEDPEVKFNWYVDGV | |
| | | EVHNAKTKPREEQYNSTY | |
| | | RVVSVLTVLHQDWLNGKE | |
| | | YKCKVSNKALPAPIEKTI | |
| | | SKAKGQPREPQVYTLPPS | |
| | | REEMTKNQVSLTCLVKGF | |
| | | YPCDIAVEWESNGQPENN | |
| | | YKTTPPVLDSDGSFFLYS | |
| | | KLTVDKSRWQQGNVFSCS | |
| | | VMHEALHNHYTQKSLSLS | |
| | | PGK (SEQ ID NO: | |
| | | 100) | |
| MET | Telisotuzumab | QVQLVQSGAEVKKPGASV | DIVMTQSPDSLAVSLGERATIN |
| | | KVSCKASGYIFTAYTMHW | CKSSESVDSYANSFLHWYQQKP |
| | | VRQAPGQGLEWMGWIKPN | GQPPKLLIYRASTRESGVPDRF |
| | | NGLANYAQKFQGRVTMTR | SGSGSGTDFTLTISSLQAEDVA |
| | | DTSISTAYMELSRLRSDD | VYYCQQSKEDPLTFGGGTKVEI |
| | | TAVYYCARSEITTEFDYW | KRTVAAPSVFIFPPSDEQLKSG |
| | | GQGTLVTVSSASTKGPSV | TASVVCLLNNFYPREAKVQWKV |
| | | FPLAPSSKSTSGGTAALG | DNALQSGNSQESVTEQDSKDST |
| | | CLVKDYFPCPVTVSWNSG | YSLSSTLTLSKADYEKHKVYAC |
| | | ALTSGVHTFPAVLQSSGL | EVTHQGLSSPVTKSFNRGEC |
| | | YSLSSVVTVPSSSLGTQT | (SEQ ID NO: 103) |
| | | YICNVNHKPSNTKVDKRV | |
| | | EPKSCDKTHTCPPCPAPE | |
| | | LLGGPSVFLFPPKPKDTL | |
| | | MISRTPEVTCVVVDVSHE | |
| | | DPEVKFNWYVDGVEVHNA | |
| | | KTKPREEQYNSTYRVVSV | |
| | | LTVLHQDWLNGKEYKCKV | |
| | | SNKALPAPIEKTISKAKG | |
| | | QPREPQVYTLPPSREEMT | |
| | | KNQVSLTCLVKGFYPCDI | |
| | | AVEWESNGQPENNYKTTP | |
| | | PVLDSDGSFFLYSKLTVD | |
| | | KSRWQQGNVFSCSVMHEA | |
| | | LHNHYTQKSLSLSPGK | |
| | | (SEQ ID NO: 102) | |
| AXL | Enapotamab | EVQLLESGGGLVQPGGSL | EIVLTQSPGTLSLSPGERATLS |
| | | RLSCAASGFTFSSYAMNW | CRASQSVSSSYLAWYQQKPGQA |
| | | VRQAPGKGLEWVSTTSGS | PRLLIYGASSRATGIPDRESGS |
| | | GASTYYADSVKGRFTISR | GSGTDFTLTISRLEPEDFAVYY |
| | | DNSKNTLYLQMNSLRAED | COQYGSSPYTFGQGTKLEIKRT |
| | | TAVYYCAKIWIAFDIWGQ | VAAPSVFIFPPSDEQLKSGTAS |
| | | GTMVTVSSASTKGPSVFP | VVCLLNNFYPREAKVQWKVDNA |
| | | LAPSSKSTSGGTAALGCL | LOSGNSQESVTEQDSKDSTYSL |
| | | VKDYFPCPVTVSWNSGAL | SSTLTLSKADYEKHKVYACEVT |
| | | TSGVHTFPAVLQSSGLYS | HQGLSSPVTKSFNRGEC (SEQ |
| | | LSSVVTVPSSSLGTQTYI | ID NO: 105) |
| | | CNVNHKPSNTKVDKRVEP | |
| | | KSCDKTHTCPPCPAPELL | |
| | | GGPSVFLFPPKPKDTLMI | |
| | | SRTPEVTCVVVDVSHEDP | |
| | | EVKFNWYVDGVEVHNAKT | |
| | | KPREEQYNSTYRVVSVLT | |
| | | VLHQDWLNGKEYKCKVSN | |
| | | KALPAPIEKTISKAKGQP | |
| | | REPQVYTLPPSREEMTKN | |
| | | QVSLTCLVKGFYPCDIAV | |
| | | EWESNGQPENNYKTTPPV | |
| | | LDSDGSFFLYSKLTVDKS | |
| | | RWQQGNVFSCSVMHEALH | |
| | | NHYTQKSLSLSPGK | |
| | | (SEQ ID NO: 104) | |
| SLC34A2 | Lifastuzumab | EVQLVESGGGLVQPGGSL | DIQMTQSPSSLSASVGDRVTIT |
| | | RLSCAASGFSFSDFAMSW | CRSSETLVHSSGNTYLEWYQQK |
| | | VRQAPGKGLEWVATIGRV | PGKAPKLLIYRVSNRFSGVPSR |
| | | AFHTYYPDSMKGRFTISR | FSGSGSGTDFTLTISSLOPEDE |
| | | DNSKNTLYLQMNSLRAED | ATYYCFQGSFNPLTFGQGTKVE |
| | | TAVYYCARHRGFDVGHED | IKRTVAAPSVFIFPPSDEQLKS |
| | | FWGQGTLVTVSSASTKGP | GTASVVCLLNNFYPREAKVQWK |
| | | SVFPLAPSSKSTSGGTAA | VDNALQSGNSQESVTEQDSKDS |
| | | LGCLVKDYFPCPVTVSWN | TYSLSSTLTLSKADYEKHKVYA |
| | | SGALTSGVHTFPAVLQSS | CEVTHQGLSSPVTKSFNRGEC |
| | | GLYSLSSVVTVPSSSLGT | (SEQ ID NO: 107) |
| | | QTYICNVNHKPSNTKVDK | |
| | | RVEPKSCDKTHTCPPCPA | |
| | | PELLGGPSVFLFPPKPKD | |
| | | TLMISRTPEVTCVVVDVS | |
| | | HEDPEVKFNWYVDGVEVH | |
| | | NAKTKPREEQYNSTYRVV | |
| | | SVLTVLHQDWLNGKEYKC | |
| | | KVSNKALPAPIEKTISKA | |
| | | KGQPREPQVYTLPPSREE | |
| | | MTKNQVSLTCLVKGFYPC | |
| | | DIAVEWESNGQPENNYKT | |
| | | TPPVLDSDGSFFLYSKLT | |
| | | VDKSRWQQGNVFSCSVMH | |
| | | EALHNHYTQKSLSLSPGK | |
| | | (SEQ ID NO: 106) | |
| NECTIN4 | Enfortumab | EVQLVESGGGLVQPGGSL | DIQMTQSPSSVSASVGDRVTIT |
| | | RLSCAASGFTFSSYNMNW | CRASQGISGWLAWYQQKPGKAP |
| | | VRQAPGKGLEWVSYISSS | KFLIYAASTLQSGVPSRFSGSG |
| | | SSTIYYADSVKGRFTISR | SGTDFTLTISSLQPEDFATYYC |
| | | DNAKNSLSLQMNSLRDED | QQANSFPPTFGGGTKVEIKRTV |
| | | TAVYYCARAYYYGMDVWG | AAPSVFIFPPSDEQLKSGTASV |
| | | QGTTVTVSSASTKGPSVF | VCLLNNFYPREAKVQWKVDNAL |
| | | PLAPSSKSTSGGTAALGC | QSGNSQESVTEQDSKDSTYSLS |
| | | LVKDYFPCPVTVSWNSGA | STLTLSKADYEKHKVYACEVTH |
| | | LTSGVHTFPAVLQSSGLY | QGLSSPVTKSFNRGEC (SEQ |
| | | SLSSVVTVPSSSLGTQTY | ID NO: 109) |
| | | ICNVNHKPSNTKVDKRVE | |
| | | PKSCDKTHTCPPCPAPEL | |
| | | LGGPSVFLFPPKPKDTLM | |
| | | ISRTPEVTCVVVDVSHED | |
| | | PEVKFNWYVDGVEVHNAK | |
| | | TKPREEQYNSTYRVVSVL | |
| | | TVLHQDWLNGKEYKCKVS | |
| | | NKALPAPIEKTISKAKGQ | |
| | | PREPQVYTLPPSREEMTK | |
| | | NQVSLTCLVKGFYPCDIA | |
| | | VEWESNGQPENNYKTTPP | |
| | | VLDSDGSFFLYSKLTVDK | |
| | | SRWQQGNVFSCSVMHEAL | |
| | | HNHYTQKSLSLSPGK | |
| | | (SEQ ID NO: 108) | |
| TACSTD2 | Sacituzumab | QVQLQQSGSELKKPGASV | DIQLTQSPSSLSASVGDRVSIT |
| | | KVSCKASGYTFTNYGMNW | CKASQDVSIAVAWYQQKPGKAP |
| | | VKQAPGQGLKWMGWINTY | KLLIYSASYRYTGVPDRFSGSG |
| | | TGEPTYTDDFKGRFAFSL | SGTDFTLTISSLOPEDFAVYYC |
| | | DTSVSTAYLQISSLKADD | QQHYITPLTFGAGTKVEIKRTV |
| | | TAVYFCARGGFGSSYWYF | AAPSVFIFPPSDEQLKSGTASV |
| | | DVWGQGSLVTVSSASTKG | VCLLNNFYPREAKVQWKVDNAL |
| | | PSVFPLAPSSKSTSGGTA | QSGNSQESVTEQDSKDSTYSLS |
| | | ALGCLVKDYFPCPVTVSW | STLTLSKADYEKHKVYACEVTH |
| | | NSGALTSGVHTFPAVLQS | QGLSSPVTKSFNRGEC (SEQ |
| | | SGLYSLSSVVTVPSSSLG | ID NO: 111) |
| | | TQTYICNVNHKPSNTKVD | |
| | | KRVEPKSCDKTHTCPPCP | |
| | | APELLGGPSVFLFPPKPK | |
| | | DTLMISRTPEVTCVVVDV | |
| | | SHEDPEVKFNWYVDGVEV | |
| | | HNAKTKPREEQYNSTYRV | |
| | | VSVLTVLHQDWLNGKEYK | |
| | | CKVSNKALPAPIEKTISK | |
| | | AKGQPREPQVYTLPPSRE | |
| | | EMTKNQVSLTCLVKGFYP | |
| | | CDIAVEWESNGQPENNYK | |
| | | TTPPVLDSDGSFFLYSKL | |
| | | TVDKSRWQQGNVFSCSVM | |
| | | HEALHNHYTQKSLSLSPG | |
| | | K (SEQ ID NO: 110) | |
| SLC39A6 | Ladiratuzumab | QVQLVQSGAEVKKPGASV | DVVMTQSPLSLPVTLGQPASIS |
| | | KVSCKASGLTIEDYYMHW | CRSSQSLLHSSGNTYLEWYQQR |
| | | VRQAPGQGLEWMGWIDPE | PGQSPRPLIYKISTRFSGVPDR |
| | | NGDTEYGPKFQGRVTMTR | FSGSGSGTDFTLKISRVEAEDV |
| | | DTSINTAYMELSRLRSDD | GVYYCFQGSHVPYTFGGGTKVE |
| | | TAVYYCAVHNAHYGTWFA | IKRTVAAPSVFIFPPSDEQLKS |
| | | YWGQGTLVTVSSASTKGP | GTASVVCLLNNFYPREAKVQWK |
| | | SVFPLAPSSKSTSGGTAA | VDNALQSGNSQESVTEQDSKDS |
| | | LGCLVKDYFPCPVTVSWN | TYSLSSTLTLSKADYEKHKVYA |
| | | SGALTSGVHTFPAVLQSS | CEVTHQGLSSPVTKSFNRGEC |
| | | GLYSLSSVVTVPSSSLGT | (SEQ ID NO: 113) |
| | | QTYICNVNHKPSNTKVDK | |
| | | RVEPKSCDKTHTCPPCPA | |
| | | PELLGGPSVFLFPPKPKD | |
| | | TLMISRTPEVTCVVVDVS | |
| | | HEDPEVKFNWYVDGVEVH | |
| | | NAKTKPREEQYNSTYRVV | |
| | | SVLTVLHQDWLNGKEYKC | |
| | | KVSNKALPAPIEKTISKA | |
| | | KGQPREPQVYTLPPSREE | |
| | | MTKNQVSLTCLVKGFYPC | |
| | | DIAVEWESNGQPENNYKT | |
| | | TPPVLDSDGSFFLYSKLT | |
| | | VDKSRWQQGNVFSCSVMH | |
| | | EALHNHYTQKSLSLSPGK | |
| | | (SEQ ID NO: 112) | |
| GPNMB | Glembatumumab | QVQLQESGPGLVKPSQTL | EIVMTQSPATLSVSPGERATLS |
| | | SLTCTVSGGSISSFNYYW | CRASQSVDNNLVWYQQKPGQAP |
| | | SWIRHHPGKGLEWIGYIY | RLLIYGASTRATGIPARFSGSG |
| | | YSGSTYSNPSLKSRVTIS | SGTEFTLTISSLQSEDFAVYYC |
| | | VDTSKNQFSLTLSSVTAA | QQYNNWPPWTFGQGTKVEIKRT |
| | | DTAVYYCARGYNWNYFDY | VAAPSVFIFPPSDEQLKSGTAS |
| | | WGQGTLVTVSSASTKGPS | VVCLLNNFYPREAKVQWKVDNA |
| | | VFPLAPSSKSTSGGTAAL | LQSGNSQESVTEQDSKDSTYSL |
| | | GCLVKDYFPCPVTVSWNS | SSTLTLSKADYEKHKVYACEVT |
| | | GALTSGVHTFPAVLQSSG | HQGLSSPVTKSENRGEC (SEQ |
| | | LYSLSSVVTVPSSSLGTQ | ID NO: 115) |
| | | TYICNVNHKPSNTKVDKR | |
| | | VEPKSCDKTHTCPPCPAP | |
| | | ELLGGPSVFLFPPKPKDT | |
| | | LMISRTPEVTCVVVDVSH | |
| | | EDPEVKFNWYVDGVEVHN | |
| | | AKTKPREEQYNSTYRVVS | |
| | | VLTVLHQDWLNGKEYKCK | |
| | | VSNKALPAPIEKTISKAK | |
| | | GQPREPQVYTLPPSREEM | |
| | | TKNQVSLTCLVKGFYPCD | |
| | | IAVEWESNGQPENNYKTT | |
| | | PPVLDSDGSFFLYSKLTV | |
| | | DKSRWQQGNVFSCSVMHE | |
| | | ALHNHYTQKSLSLSPGK | |
| | | (SEQ ID NO: 114) | |
| MSLN | Anetumab | QVELVOSGAEVKKPGESL | DIALTQPASVSGSPGQSITISC |
| | | KISCKGSGYSFTSYWIGW | TGTSSDIGGYNSVSWYQQHPGK |
| | | VRQAPGKGLEWMGIIDPG | APKLMIYGVNNRPSGVSNRFSG |
| | | DSRTRYSPSFQGQVTISA | SKSGNTASLTISGLQAEDEADY |
| | | DKSISTAYLOWSSLKASD | YCSSYDIESATPVFGGGTKLTV |
| | | TAMYYCARGQLYGGTYMD | LGQPKAAPSVTLFPPSSEELQA |
| | | GWGQGTLVTVSSASTKGP | NKATLVCLISDFYPGAVTVAWK |
| | | SVFPLAPSSKSTSGGTAA | ADSSPVKAGVETTTPSKQSNNK |
| | | LGCLVKDYFPCPVTVSWN | YAASSYLSLTPEQWKSHRSYSC |
| | | SGALTSGVHTFPAVLOSS | QVTHEGSTVEKTVAPTECS |
| | | GLYSLSSVVTVPSSSLGT | (SEQ ID NO: 117) |
| | | QTYICNVNHKPSNTKVDK | |
| | | RVEPKSCDKTHTCPPCPA | |
| | | PELLGGPSVFLFPPKPKD | |
| | | TLMISRTPEVTCVVVDVS | |
| | | HEDPEVKFNWYVDGVEVH | |
| | | NAKTKPREEQYNSTYRVV | |
| | | SVLTVLHQDWLNGKEYKC | |
| | | KVSNKALPAPIEKTISKA | |
| | | KGQPREPQVYTLPPSREE | |
| | | MTKNQVSLTCLVKGFYPC | |
| | | DIAVEWESNGQPENNYKT | |
| | | TPPVLDSDGSFFLYSKLT | |
| | | VDKSRWQQGNVFSCSVMH | |
| | | EALHNHYTQKSLSLSPGK | |
| | | (SEQ ID NO: 116) | |
| CD74 | Milatuzumab | QVQLQQSGSELKKPGASV | DIQLTQSPLSLPVTLGQPASIS |
| | | KVSCKASGYTFTNYGVNW | CRSSQSLVHRNGNTYLHWFQQR |
| | | IKQAPGQGLQWMGWINPN | PGQSPRLLIYTVSNRFSGVPDR |
| | | TGEPTFDDDFKGRFAFSL | FSGSGSGTDFTLKISRVEAEDV |
| | | DTSVSTAYLQISSLKADD | GVYFCSQSSHVPPTFGAGTRLE |
| | | TAVYFCSRSRGKNEAWFA | IKRTVAAPSVFIFPPSDEQLKS |
| | | YWGQGTLVTVSSASTKGP | GTASVVCLLNNFYPREAKVQWK |
| | | SVFPLAPSSKSTSGGTAA | VDNALQSGNSQESVTEQDSKDS |
| | | LGCLVKDYFPCPVTVSWN | TYSLSSTLTLSKADYEKHKVYA |
| | | SGALTSGVHTFPAVLQSS | CEVTHQGLSSPVTKSENRGEC |
| | | GLYSLSSVVTVPSSSLGT | (SEQ ID NO: 119) |
| | | QTYICNVNHKPSNTKVDK | |
| | | RVEPKSCDKTHTCPPCPA | |
| | | PELLGGPSVFLFPPKPKD | |
| | | TLMISRTPEVTCVVVDVS | |
| | | HEDPEVKFNWYVDGVEVH | |
| | | NAKTKPREEQYNSTYRVV | |
| | | SVLTVLHQDWLNGKEYKC | |
| | | KVSNKALPAPIEKTISKA | |
| | | KGQPREPQVYTLPPSREE | |
| | | MTKNQVSLTCLVKGFYPC | |
| | | DIAVEWESNGQPENNYKT | |
| | | TPPVLDSDGSFFLYSKLT | |
| | | VDKSRWQQGNVFSCSVMH | |
| | | EALHNHYTQKSLSLSPGK | |
| | | (SEQ ID NO: 118) | |
| F3 | Tisotumab | EVQLLESGGGLVQPGGSL | DIQMTQSPPSLSASAGDRVTIT |
| | | RLSCAASGFTFSNYAMSW | CRASQGISSRLAWYQQKPEKAP |
| | | VRQAPGKGLEWVSSISGS | KSLIYAASSLQSGVPSRFSGSG |
| | | GDYTYYTDSVKGRFTISR | SGTDFTLTISSLOPEDFATYYC |
| | | DNSKNTLYLQMNSLRAED | QQYNSYPYTFGQGTKLEIKRTV |
| | | TAVYYCARSPWGYYLDSW | AAPSVFIFPPSDEQLKSGTASV |
| | | GQGTLVTVSSASTKGPSV | VCLLNNFYPREAKVQWKVDNAL |
| | | FPLAPSSKSTSGGTAALG | QSGNSQESVTEQDSKDSTYSLS |
| | | CLVKDYFPCPVTVSWNSG | STLTLSKADYEKHKVYACEVTH |
| | | ALTSGVHTFPAVLQSSGL | QGLSSPVTKSFNRGEC (SEQ |
| | | YSLSSVVTVPSSSLGTQT | ID NO:121) |
| | | YICNVNHKPSNTKVDKRV | |
| | | EPKSCDKTHTCPPCPAPE | |
| | | LLGGPSVFLFPPKPKDTL | |
| | | MISRTPEVTCVVVDVSHE | |
| | | DPEVKFNWYVDGVEVHNA | |
| | | KTKPREEQYNSTYRVVSV | |
| | | LTVLHQDWLNGKEYKCKV | |
| | | SNKALPAPIEKTISKAKG | |
| | | QPREPQVYTLPPSREEMT | |
| | | KNQVSLTCLVKGFYPCDI | |
| | | AVEWESNGQPENNYKTTP | |
| | | PVLDSDGSFFLYSKLTVD | |
| | | KSRWQQGNVFSCSVMHEA | |
| | | LHNHYTQKSLSLSPGK | |
| | | (SEQ ID NO: 120) | |
| MUC16 | Sofituzumab | EVQLVESGGGLVQPGGSL | DIQMTQSPSSLSASVGDRVTIT |
| | | RLSCAASGYSITNDYAWN | CKASDLIHNWLAWYQQKPGKAP |
| | | WVRQAPGKGLEWVGYISY | KLLIYGATSLETGVPSRFSGSG |
| | | SGYTTYNPSLKSRFTISR | SGTDETLTISSLQPEDFATYYC |
| | | DTSKNTLYLQMNSLRAED | QQYWTTPFTFGQGTKVEIKRTV |
| | | TAVYYCARWTSGLDYWGQ | AAPSVFIFPPSDEQLKSGTASV |
| | | GTLVTVSSASTKGPSVFP | VCLLNNFYPREAKVQWKVDNAL |
| | | LAPSSKSTSGGTAALGCL | QSGNSQESVTEQDSKDSTYSLS |
| | | VKDYFPCPVTVSWNSGAL | STLTLSKADYEKHKVYACEVTH |
| | | TSGVHTFPAVLQSSGLYS | QGLSSPVTKSFNRGEC (SEQ |
| | | LSSVVTVPSSSLGTQTYI | ID NO:123) |
| | | CNVNHKPSNTKVDKRVEP | |
| | | KSCDKTHTCPPCPAPELL | |
| | | GGPSVFLFPPKPKDTLMI | |
| | | SRTPEVTCVVVDVSHEDP | |
| | | EVKFNWYVDGVEVHNAKT | |
| | | KPREEQYNSTYRVVSVLT | |
| | | VLHQDWLNGKEYKCKVSN | |
| | | KALPAPIEKTISKAKGQP | |
| | | REPQVYTLPPSREEMTKN | |
| | | QVSLTCLVKGFYPCDIAV | |
| | | EWESNGQPENNYKTTPPV | |
| | | LDSDGSFFLYSKLTVDKS | |
| | | RWQQGNVFSCSVMHEALH | |
| | | NHYTQKSLSLSPGK | |
| | | (SEQ ID NO: 122) | |
| EGFR2 | Aba | EVQLQESGPGLVKPSQTL | DIQMTQSPSSMSVSVGDRVTIT |
| | | SLTCTVSGYSISRDFAWN | CHSSQDINSNIGWLQQKPGKSF |
| | | WIRQPPGKGLEWMGYISY | KGLIYHGTNLDDGVPSRFSGSG |
| | | NGNTRYQPSLKSRITISR | SGTDYTLTISSLQPEDFATYYC |
| | | DTSKNQFFLKLNSVTAAD | VQYAQFPWTFGGGTKLEIKRTV |
| | | TATYYCVTASRGFPYWGQ | AAPSVFIFPPSDEQLKSGTASV |
| | | GTLVTVSSASTKGPSVFP | VCLLNNFYPREAKVQWKVDNAL |
| | | LAPSSKSTSGGTAALGCL | QSGNSQESVTEQDSKDSTYSLS |
| | | VKDYFPCPVTVSWNSGAL | STLTLSKADYEKHKVYACEVTH |
| | | TSGVHTFPAVLQSSGLYS | QGLSSPVTKSENRGEC (SEQ |
| | | LSSVVTVPSSSLGTQTYI | ID NO: 125) |
| | | CNVNHKPSNTKVDKRVEP | |
| | | KSCDKTHTCPPCPAPELL | |
| | | GGPSVFLFPPKPKDTLMI | |
| | | SRTPEVTCVVVDVSHEDP | |
| | | EVKFNWYVDGVEVHNAKT | |
| | | KPREEQYNSTYRVVSVLT | |
| | | VLHQDWLNGKEYKCKVSN | |
| | | KALPAPIEKTISKAKGQP | |
| | | REPQVYTLPPSREEMTKN | |
| | | QVSLTCLVKGFYPCDIAV | |
| | | EWESNGQPENNYKTTPPV | |
| | | LDSDGSFFLYSKLTVDKS | |
| | | RWQQGNVFSCSVMHEALH | |
| | | NHYTQKSLSLSPGK | |
| | | (SEQ ID NO: 124) | |
| CD7 | Ab D | EVQLVESGGGLVKPGGSL | DIQMTQTTSSLSASLGDRVTIS |
| | | KLSCAASGLTFSSYAMSW | CSASQGISNYLNWYQQKPDGTV |
| | | VRQTPEKRLEWVASISSG | KLLIYYTSSLHSGVPSRFSGSG |
| | | GFTYYPDSVKGRFTISRD | SGTDYSLTISNLEPEDIATYYC |
| | | NARNILYLQMSSLRSEDT | QQYSKLPYTFGGGTKLEIKRTV |
| | | AMYYCARDEVRGYLDVWG | AAPSVFIFPPSDEQLKSGTASV |
| | | AGTTVTVSSASTKGPSVF | VCLLNNFYPREAKVQWKVDNAL |
| | | PLAPSSKSTSGGTAALGC | QSGNSQESVTEQDSKDSTYSLS |
| | | LVKDYFPCPVTVSWNSGA | STLTLSKADYEKHKVYACEVTH |
| | | LTSGVHTFPAVLQSSGLY | QGLSSPVTKSFNRGEC (SEQ |
| | | SLSSVVTVPSSSLGTQTY | ID NO: 144) |
| | | ICNVNHKPSNTKVDKRVE | |
| | | PKSCDKTHTCPPCPAPEL | |
| | | LGGPSVFLFPPKPKDTLM | |
| | | ISRTPEVTCVVVDVSHED | |
| | | PEVKFNWYVDGVEVHNAK | |
| | | TKPREEQYNSTYRVVSVL | |
| | | TVLHQDWLNGKEYKCKVS | |
| | | NKALPAPIEKTISKAKGQ | |
| | | PREPQVYTLPPSREEMTK | |
| | | NQVSLTCLVKGFYPCDIA | |
| | | VEWESNGQPENNYKTTPP | |
| | | VLDSDGSFFLYSKLTVDK | |
| | | SRWQQGNVFSCSVMHEAL | |
| | | HNHYTQKSLSLSPGK | |
| | | (SEQ ID NO: 143) | |

TABLE D6
Exemplary Bcl-xL and target antigen amino acid sequences

| Bcl-xL/Antigen | SEQ ID NO | Amino acid sequence |
|---|---|---|
| Bcl-xL | 71 | MSQSNRELVVDFLSYKLSQKGYSWSQFSDVEENRTEAPEGTESEM |
| | | ETPSAINGNPSWHLADSPAVNGATGHSSSLDAREVIPMAAVKQAL |
| | | REAGDEFELRYRRAFSDLTSQLHITPGTAYQSFEQVVNELFRDGV |
| | | NWGRIVAFFSFGGALCVESVDKEMQVLVSRIAAWMATYLNDHLEP |
| | | WIQENGGWDTFVELYGNNAAAESRKGQERFNRWFLTGMTVAGVVL |
| | | LGSLFSRK |
| BCMA | 72 | MLQMAGQCSQNEYFDSLLHACIPCQLRCSSNTPPLTCQRYCNASV |
| | | TNSVKGTNAILWTCLGLSLIISLAVFVLMFLLRKINSEPLKDEFK |
| | | NTGSGLLGMANIDLEKSRTGDEIILPRGLEYTVEECTCEDCIKSK |
| | | PKVDSDHCFPLPAMEEGATILVTTKTNDYCKSLPAALSATEIEKS |
| | | ISAR |
| CD33 | 73 | MPLLLLLPLLWAGALAMDPNFWLQVQESVTVQEGLCVLVPCTFFH |
| | | PIPYYDKNSPVHGYWFREGAIISRDSPVATNKLDQEVQEETQGRF |
| | | RLLGDPSRNNCSLSIVDARRRDNGSYFFRMERGSTKYSYKSPQLS |
| | | VHVTDLTHRPKILIPGTLEPGHSKNLTCSVSWACEQGTPPIFSWL |
| | | SAAPTSLGPRITHSSVLIITPRPQDHGTNLTCQVKFAGAGVTTER |
| | | TIQLNVTYVPQNPTTGIFPGDGSGKQETRAGVVHGAIGGAGVTAL |
| | | LALCLCLIFFIVKTHRRKAARTAVGRNDTHPTTGSASPKHQKKSK |
| | | LHGPTETSSCSGAAPTVEMDEELHYASLNFHGMNPSKDTSTEYSE |
| | | VRTQ |
| PCAD | 74 | MGLPRGPLASLLLLQVCWLQCAASEPCRAVFREAEVTLEAGGAEQ |
| | | EPGQALGKVFMGCPGQEPALFSTDNDDFTVRNGETVQERRSLKER |
| | | NPLKIFPSKRILRRHKRDWVVAPISVPENGKGPFPQRLNQLKSNK |
| | | DRDTKIFYSITGPGADSPPEGVFAVEKETGWLLLNKPLDREEIAK |
| | | YELFGHAVSENGASVEDPMNISIIVTDQNDHKPKFTQDTFRGSVL |
| | | EGVLPGTSVMQVTATDEDDAIYTYNGVVAYSIHSQEPKDPHDLMF |
| | | TIHRSTGTISVISSGLDREKVPEYTLTIQATDMDGDGSTTTAVAV |
| | | VEILDANDNAPMFDPQKYEAHVPENAVGHEVQRLTVTDLDAPNSP |
| | | AWRATYLIMGGDDGDHFTITTHPESNQGILTTRKGLDFEAKNQHT |
| | | LYVEVTNEAPFVLKLPTSTATIVVHVEDVNEAPVFVPPSKVVEVQ |
| | | EGIPTGEPVCVYTAEDPDKENQKISYRILRDPAGWLAMDPDSGQV |
| | | TAVGTLDREDEQFVRNNIYEVMVLAMDNGSPPTTGTGTLLLTLID |
| | | VNDHGPVPEPRQITICNQSPVRQVLNITDKDLSPHTSPFQAQLTD |
| | | DSDIYWTAEVNEEGDTVVLSLKKFLKQDTYDVHLSLSDHGNKEQL |
| | | TVIRATVCDCHGHVETCPGPWKGGFILPVLGAVLALLFLLLVLLL |
| | | LVRKKRKIKEPLLLPEDDTRDNVFYYGEEGGGEEDQDYDITQLHR |
| | | GLEARPEVVLRNDVAPTIIPTPMYRPRPANPDEIGNFIIENLKAA |
| | | NTDPTAPPYDTLLVEDYEGSGSDAASLSSLTSSASDQDQDYDYLN |
| | | EWGSRFKKLADMYGGGEDD |
| HER2/NEU | 75 | MELAALCRWGLLLALLPPGAASTQVCTGTDMKLRLPASPETHLDM |
| | | LRHLYQGCQVVQGNLELTYLPTNASLSFLQDIQEVQGYVLIAHNQ |
| | | VRQVPLQRLRIVRGTQLFEDNYALAVLDNGDPLNNTTPVTGASPG |
| | | GLRELQLRSLTEILKGGVLIQRNPQLCYQDTILWKDIFHKNNQLA |
| | | LTLIDTNRSRACHPCSPMCKGSRCWGESSEDCQSLTRTVCAGGCA |
| | | RCKGPLPTDCCHEQCAAGCTGPKHSDCLACLHFNHSGICELHCPA |
| | | LVTYNTDTFESMPNPEGRYTFGASCVTACPYNYLSTDVGSCTLVC |
| | | PLHNQEVTAEDGTQRCEKCSKPCARVCYGLGMEHLREVRAVTSAN |
| | | IQEFAGCKKIFGSLAFLPESEDGDPASNTAPLQPEQLQVFETLEE |
| | | ITGYLYISAWPDSLPDLSVFQNLQVIRGRILHNGAYSLTLQGLGI |
| | | SWLGLRSLRELGSGLALIHHNTHLCFVHTVPWDQLFRNPHQALLH |
| | | TANRPEDECVGEGLACHQLCARGHCWGPGPTQCVNCSQFLRGQEC |
| | | VEECRVLQGLPREYVNARHCLPCHPECQPQNGSVTCFGPEADQCV |
| | | ACAHYKDPPFCVARCPSGVKPDLSYMPIWKFPDEEGACQPCPINC |
| | | THSCVDLDDKGCPAEQRASPLTSIISAVVGILLVVVLGVVFGILI |
| | | KRRQQKIRKYTMRRLLQETELVEPLTPSGAMPNQAQMRILKETEL |
| | | RKVKVLGSGAFGTVYKGIWIPDGENVKIPVAIKVLRENTSPKANK |
| | | EILDEAYVMAGVGSPYVSRLLGICLTSTVQLVTQLMPYGCLLDHV |
| | | RENRGRLGSQDLLNWCMQIAKGMSYLEDVRLVHRDLAARNVLVKS |
| | | PNHVKITDFGLARLLDIDETEYHADGGKVPIKWMALESILRRRFT |
| | | HQSDVWSYGVTVWELMTFGAKPYDGIPAREIPDLLEKGERLPQPP |
| | | ICTIDVYMIMVKCWMIDSECRPRFRELVSEFSRMARDPQRFVVIQ |
| | | NEDLGPASPLDSTFYRSLLEDDDMGDLVDAEEYLVPQQGFFCPDP |
| | | APGAGGMVHHRHRSSSTRSGGGDLTLGLEPSEEEAPRSPLAPSEG |
| | | AGSDVFDGDLGMGAAKGLQSLPTHDPSPLQRYSEDPTVPLPSETD |
| | | GYVAPLTCSPQPEYVNQPDVRPQPPSPREGPLPAARPAGATLERP |
| | | KTLSPGKNGVVKDVFAFGGAVENPEYLTPQGGAAPQPHPPPAFSP |
| | | AFDNLYYWDQDPPERGAPPSTFKGTPTAENPEYLGLDVPV |
| CD38 | 76 | MANCEFSPVSGDKPCCRLSRRAQLCLGVSILVLILVVVLAVVVPR |
| | | WRQQWSGPGTTKRFPETVLARCVKYTEIHPEMRHVDCQSVWDAFK |
| | | GAFISKHPCNITEEDYQPLMKLGTQTVPCNKILLWSRIKDLAHQF |
| | | TQVQRDMFTLEDTLLGYLADDLTWCGEFNTSKINYQSCPDWRKDC |
| | | SNNPVSVFWKTVSRRFAEAACDVVHVMLNGSRSKIFDKNSTFGSV |
| | | EVHNLQPEKVQTLEAWVIHGGREDSRDLCQDPTIKELESIISKRN |
| | | IQFSCKNIYRPDKFLQCVKNPEDSSCTSEI |
| CD48 | 77 | MCSRGWDSCLALELLLLPLSLLVTSIQGHLVHMTVVSGSNVTLNI |
| | | SESLPENYKQLTWFYTFDQKIVEWDSRKSKYFESKFKGRVRLDPQ |
| | | SGALYISKVQKEDNSTYIMRVLKKTGNEQEWKIKLQVLDPVPKPV |
| | | IKIEKIEDMDDNCYLKLSCVIPGESVNYTWYGDKRPFPKELQNSV |
| | | LETTLMPHNYSRCYTCQVSNSVSSKNGTVCLSPPCTLARSFGVEW |
| | | IASWLVVTVPTILGLLLT |
| CD79b | 78 | MARLALSPVPSHWMVALLLLLSAEPVPAARSEDRYRNPKGSACSR |
| | | IWQSPRFIARKRGFTVKMHCYMNSASGNVSWLWKQEMDENPQQLK |
| | | LEKGRMEESQNESLATLTIQGIRFEDNGIYFCQQKCNNTSEVYQG |
| | | CGTELRVMGFSTLAQLKQRNTLKDGIIMIQTLLIILFIIVPIFLL |
| | | LDKDDSKAGMEEDHTYEGLDIDQTATYEDIVTLRTGEVKWSVGEH |
| | | PGQE |
| EGFR | 126 | MRPSGTAGAALLALLAALCPASRALEEKKVCQGTSNKLTQLGTFE |
| (NP_005219.2) | | DHFLSLQRMFNNCEVVLGNLEITYVQRNYDLSFLKTIQEVAGYVL |
| | | IALNTVERIPLENLQIIRGNMYYENSYALAVLSNYDANKTGLKEL |
| | | PMRNLQEILHGAVRFSNNPALCNVESIQWRDIVSSDFLSNMSMDF |
| | | QNHLGSCQKCDPSCPNGSCWGAGEENCQKLTKIICAQQCSGRCRG |
| | | KSPSDCCHNQCAAGCTGPRESDCLVCRKFRDEATCKDTCPPLMLY |
| | | NPTTYQMDVNPEGKYSFGATCVKKCPRNYVVTDHGSCVRACGADS |
| | | YEMEEDGVRKCKKCEGPCRKVCNGIGIGEFKDSLSINATNIKHFK |
| | | NCTSISGDLHILPVAFRGDSFTHTPPLDPQELDILKTVKEITGEL |
| | | LIQAWPENRTDLHAFENLEIIRGRTKQHGQFSLAVVSLNITSLGL |
| | | RSLKEISDGDVIISGNKNLCYANTINWKKLFGTSGQKTKIISNRG |
| | | ENSCKATGQVCHALCSPEGCWGPEPRDCVSCRNVSRGRECVDKCN |
| | | LLEGEPREFVENSECIQCHPECLPQAMNITCTGRGPDNCIQCAHY |
| | | IDGPHCVKTCPAGVMGENNTLVWKYADAGHVCHLCHPNCTYGCTG |
| | | PGLEGCPTNGPKIPSIATGMVGALLLLLVVALGIGLFMRRRHIVR |
| | | KRTLRRLLQERELVEPLTPSGEAPNQALLRILKETEFKKIKVLGS |
| | | GAFGTVYKGLWIPEGEKVKIPVAIKELREATSPKANKEILDEAYV |
| | | MASVDNPHVCRLLGICLTSTVQLITQLMPFGCLLDYVREHKDNIG |
| | | SQYLLNWCVQIAKGMNYLEDRRLVHRDLAARNVLVKTPQHVKITD |
| | | FGLAKLLGAEEKEYHAEGGKVPIKWMALESILHRIYTHQSDVWSY |
| | | GVTVWELMTFGSKPYDGIPASEISSILEKGERLPQPPICTIDVYM |
| | | IMVKCWMIDADSRPKFRELIIEFSKMARDPQRYLVIQGDERMHLP |
| | | SPTDSNFYRALMDEEDMDDVVDADEYLIPQQGFFSSPSTSRTPLL |
| | | SSLSATSNNSTVACIDRNGLQSCPIKEDSFLQRYSSDPTGALTED |
| | | SIDDTFLPVPEYINQSVPKRPAGSVQNPVYHNQPLNPAPSRDPHY |
| | | QDPHSTAVGNPEYLNTVQPTCVNSTFDSPAHWAQKGSHQISLDNP |
| | | DYQQDFFPKEAKPNGIFKGSTAENAEYLRVAPQSSEFIGA |
| CD7 | 127 | MAGPPRLLLLPLLLALARGLPGALAAQEVQQSPHCTTVPVGASVN |
| (NP_006128.1) | | ITCSTSGGLRGIYLRQLGPQPQDIIYYEDGVVPTTDRRFRGRIDF |
| | | SGSQDNLTITMHRLQLSDTGTYTCQAITEVNVYGSGTLVLVTEEQ |
| | | SQGWHRCSDAPPRASALPAPPTGSALPDPQTASALPDPPAASALP |
| | | AALAVISFLLGLGLGVACVLARTQIKKLCSWRDKNSAACVVYEDM |
| | | SHSRCNTLSSPNQYQ |
| TFRC | 128 | MMDQARSAFSNLFGGEPLSYTRFSLARQVDGDNSHVEMKLAVDEE |
| (NP_003225.2) | | ENADNNTKANVTKPKRCSGSICYGTIAVIVFFLIGFMIGYLGYCK |
| | | GVEPKTECERLAGTESPVREEPGEDFPAARRLYWDDLKRKLSEKL |
| | | DSTDFTGTIKLLNENSYVPREAGSQKDENLALYVENQFREFKLSK |
| | | VWRDQHFVKIQVKDSAQNSVIIVDKNGRLVYLVENPGGYVAYSKA |
| | | ATVTGKLVHANFGTKKDFEDLYTPVNGSIVIVRAGKITFAEKVAN |
| | | AESLNAIGVLIYMDQTKFPIVNAELSFFGHAHLGTGDPYTPGFPS |
| | | FNHTQFPPSRSSGLPNIPVQTISRAAAEKLFGNMEGDCPSDWKTD |
| | | STCRMVTSESKNVKLTVSNVLKEIKILNIFGVIKGFVEPDHYVVV |
| | | GAQRDAWGPGAAKSGVGTALLLKLAQMFSDMVLKDGFQPSRSIIF |
| | | ASWSAGDFGSVGATEWLEGYLSSLHLKAFTYINLDKAVLGTSNFK |
| | | VSASPLLYTLIEKTMQNVKHPVTGQFLYQDSNWASKVEKLTLDNA |
| | | AFPFLAYSGIPAVSFCFCEDTDYPYLGTTMDTYKELIERIPELNK |
| | | VARAAAEVAGQFVIKLTHDVELNLDYERYNSQLLSFVRDLNQYRA |
| | | DIKEMGLSLQWLYSARGDFFRATSRLTTDFGNAEKTDRFVMKKLN |
| | | DRVMRVEYHFLSPYVSPKESPFRHVFWGSGSHTLPALLENLKLRK |
| | | QNNGAFNETLFRNQLALATWTIQGAANALSGDVWDIDNEF |
| EPCAM | 129 | MAPPQVLAFGLLLAAATATFAAAQEECVCENYKLAVNCFVNNNRQ |
| (NP_002345.2) | | CQCTSVGAQNTVICSKLAAKCLVMKAEMNGSKLGRRAKPEGALQN |
| | | NDGLYDPDCDESGLFKAKQCNGTSMCWCVNTAGVRRTDKDTEITC |
| | | SERVRTYWIIIELKHKAREKPYDSKSLRTALQKEITTRYQLDPKF |
| | | ITSILYENNVITIDLVQNSSQKTQNDVDIADVAYYFEKDVKGESL |
| | | FHSKKMDLTVNGEQLDLDPGQTLIYYVDEKAPEFSMQGLKAGVIA |
| | | VIVVVVIAVVAGIVVLVISRKKRMAKYEKAEIKEMGEMHRELNA |
| FOLR1 | 130 | MAQRMTTQLLLLLVWVAVVGEAQTRIAWARTELLNVCMNAKHHKE |
| (NP_057936.1) | | KPGPEDKLHEQCRPWRKNACCSTNTSQEAHKDVSYLYRENWNHCG |
| | | EMAPACKRHFIQDTCLYECSPNLGPWIQQVDQSWRKERVLNVPLC |
| | | KEDCEQWWEDCRTSYTCKSNWHKGWNWTSGENKCAVGAACQPFHF |
| | | YFPTPTVLCNEIWTHSYKVSNYSRGSGRCIQMWFDPAQGNPNEEV |
| | | ARFYAAAMSGAGPWAAWPFLLSLALMLLWLLS |
| ENPP3 | 131 | MESTLTLATEQPVKKNTLKKYKIACIVLLALLVIMSLGLGLGLGL |
| (NP_005012.2) | | RKLEKQGSCRKKCFDASFRGLENCRCDVACKDRGDCCWDFEDTCV |
| | | ESTRIWMCNKFRCGETRLEASLCSCSDDCLQRKDCCADYKSVCQG |
| | | ETSWLEENCDTAQQSQCPEGFDLPPVILFSMDGFRAEYLYTWDTL |
| | | MPNINKLKTCGIHSKYMRAMYPTKTFPNHYTIVTGLYPESHGIID |
| | | NNMYDVNLNKNFSLSSKEQNNPAWWHGQPMWLTAMYQGLKAATYF |
| | | WPGSEVAINGSFPSIYMPYNGSVPFEERISTLLKWLDLPKAERPR |
| | | FYTMYFEEPDSSGHAGGPVSARVIKALQVVDHAFGMLMEGLKQRN |
| | | LHNCVNIILLADHGMDQTYCNKMEYMTDYFPRINFFYMYEGPAPR |
| | | IRAHNIPHDFFSFNSEEIVRNLSCRKPDQHFKPYLTPDLPKRLHY |
| | | AKNVRIDKVHLFVDQQWLAVRSKSNTNCGGGNHGYNNEFRSMEAI |
| | | FLAHGPSFKEKTEVEPFENIEVYNLMCDLLRIQPAPNNGTHGSLN |
| | | HLLKVPFYEPSHAEEVSKFSVCGFANPLPTESLDCFCPHLQNSTQ |
| | | LEQVNQMLNLTQEEITATVKVNLPFGRPRVLQKNVDHCLLYHREY |
| | | VSGFGKAMRMPMWSSYTVPQLGDTSPLPPTVPDCLRADVRVPPSE |
| | | SQKCSFYLADKNITHGFLYPPASNRTSDSQYDALITSNLVPMYEE |
| | | FRKMWDYFHSVLLIKHATERNGVNVVSGPIFDYNYDGHFDAPDEI |
| | | TKHLANTDVPIPTHYFVVLTSCKNKSHTPENCPGWLDVLPFIIPH |
| | | RPTNVESCPEGKPEALWVEERFTAHIARVRDVELLTGLDFYQDKV |
| | | QPVSEILQLKTYLPTFETTI |
| MET | 132 | MKAPAVLAPGILVLLFTLVQRSNGECKEALAKSEMNVNMKYQLPN |
| (NP_001120972.1) | | FTAETPIQNVILHEHHIFLGATNYIYVLNEEDLQKVAEYKTGPVL |
| | | EHPDCFPCQDCSSKANLSGGVWKDNINMALVVDTYYDDQLISCGS |
| | | VNRGTCQRHVFPHNHTADIQSEVHCIFSPQIEEPSQCPDCVVSAL |
| | | GAKVLSSVKDRFINFFVGNTINSSYFPDHPLHSISVRRLKETKDG |
| | | FMFLTDQSYIDVLPEFRDSYPIKYVHAFESNNFIYFLTVQRETLD |
| | | AQTFHTRIIRFCSINSGLHSYMEMPLECILTEKRKKRSTKKEVEN |
| | | ILQAAYVSKPGAQLARQIGASLNDDILFGVFAQSKPDSAEPMDRS |
| | | AMCAFPIKYVNDFFNKIVNKNNVRCLQHFYGPNHEHCENRTLLRN |
| | | SSGCEARRDEYRTEFTTALQRVDLFMGQFSEVLLTSISTFIKGDL |
| | | TIANLGTSEGRFMQVVVSRSGPSTPHVNFLLDSHPVSPEVIVEHT |
| | | LNQNGYTLVITGKKITKIPLNGLGCRHFQSCSQCLSAPPFVQCGW |
| | | CHDKCVRSEECLSGTWTQQICLPAIYKVFPNSAPLEGGTRLTICG |
| | | WDFGFRRNNKFDLKKTRVLLGNESCTLTLSESTMNTLKCTVGPAM |
| | | NKHFNMSIIISNGHGTTQYSTFSYVDPVITSISPKYGPMAGGTLL |
| | | TLTGNYLNSGNSRHISIGGKTCTLKSVSNSILECYTPAQTISTEF |
| | | AVKLKIDLANRETSIFSYREDPIVYEIHPTKSFISTWWKEPLNIV |
| | | SFLFCFASGGSTITGVGKNLNSVSVPRMVINVHEAGRNFTVACQH |
| | | RSNSEIICCTTPSLQQLNLQLPLKTKAFFMLDGILSKYFDLIYVH |
| | | NPVFKPFEKPVMISMGNENVLEIKGNDIDPEAVKGEVLKVGNKSC |
| | | ENIHLHSEAVLCTVPNDLLKLNSELNIEWKQAISSTVLGKVIVQP |
| | | DQNFTGLIAGVVSISTALLLLLGFFLWLKKRKQIKDLGSELVRYD |
| | | ARVHTPHLDRLVSARSVSPTTEMVSNESVDYRATFPEDQFPNSSQ |
| | | NGSCRQVQYPLTDMSPILTSGDSDISSPLLQNTVHIDLSALNPEL |
| | | VQAVQHVVIGPSSLIVHFNEVIGRGHFGCVYHGTLLDNDGKKIHC |
| | | AVKSLNRITDIGEVSQFLTEGIIMKDFSHPNVLSLLGICLRSEGS |
| | | PLVVLPYMKHGDLRNFIRNETHNPTVKDLIGFGLQVAKGMKYLAS |
| | | KKFVHRDLAARNCMLDEKFTVKVADFGLARDMYDKEYYSVHNKTG |
| | | AKLPVKWMALESLQTQKFTTKSDVWSFGVLLWELMTRGAPPYPDV |
| | | NTFDITVYLLQGRRLLQPEYCPDPLYEVMLKCWHPKAEMRPSFSE |
| | | LVSRISAIFSTFIGEHYVHVNATYVNVKCVAPYPSLLSSEDNADD |
| | | EVDTRPASFWETS |
| AXL | 133 | MAWRCPRMGRVPLAWCLALCGWACMAPRGTQAEESPFVGNPGNIT |
| (NP_068713.2) | | GARGLTGTLRCQLQVQGEPPEVHWLRDGQILELADSTQTQVPLGE |
| | | DEQDDWIVVSQLRITSLQLSDTGQYQCLVFLGHQTFVSQPGYVGL |
| | | EGLPYFLEEPEDRTVAANTPFNLSCQAQGPPEPVDLLWLQDAVPL |
| | | ATAPGHGPQRSLHVPGLNKTSSFSCEAHNAKGVTTSRTATITVLP |
| | | QQPRNLHLVSRQPTELEVAWTPGLSGIYPLTHCTLQAVLSDDGMG |
| | | IQAGEPDPPEEPLTSQASVPPHQLRLGSLHPHTPYHIRVACTSSQ |
| | | GPSSWTHWLPVETPEGVPLGPPENISATRNGSQAFVHWQEPRAPL |
| | | QGTLLGYRLAYQGQDTPEVLMDIGLRQEVTLELQGDGSVSNLTVC |
| | | VAAYTAAGDGPWSLPVPLEAWRPGQAQPVHQLVKEPSTPAFSWPW |
| | | WYVLLGAVVAAACVLILALFLVHRRKKETRYGEVFEPTVERGELV |
| | | VRYRVRKSYSRRTTEATLNSLGISEELKEKLRDVMVDRHKVALGK |
| | | TLGEGEFGAVMEGQLNQDDSILKVAVKTMKIAICTRSELEDELSE |
| | | AVCMKEFDHPNVMRLIGVCFQGSERESFPAPVVILPFMKHGDLHS |
| | | FLLYSRLGDQPVYLPTQMLVKFMADIASGMEYLSTKRFIHRDLAA |
| | | RNCMLNENMSVCVADFGLSKKIYNGDYYRQGRIAKMPVKWIAIES |
| | | LADRVYTSKSDVWSFGVTMWEIATRGQTPYPGVENSEIYDYLRQG |
| | | NRLKQPADCLDGLYALMSRCWELNPQDRPSFTELREDLENTLKAL |
| | | PPAQEPDEILYVNMDEGGGYPEPPGAAGGADPPTQPDPKDSCSCL |
| | | TAAEVHPAGRYVLCPSTTPSPAQPADRGSPAAPGQEDGA |
| SLC34A2 | 134 | MAPWPELGDAQPNPDKYLEGAAGQQPTAPDKSKETNKTDNTEAPV |
| (NP_006415.3) | | TKIELLPSYSTATLIDEPTEVDDPWNLPTLQDSGIKWSERDTKGK |
| | | ILCFFQGIGRLILLLGFLYFFVCSLDILSSAFQLVGGKMAGQFFS |
| | | NSSIMSNPLLGLVIGVLVTVLVQSSSTSTSIVVSMVSSSLLTVRA |
| | | AIPIIMGANIGTSITNTIVALMQVGDRSEFRRAFAGATVHDFFNW |
| | | LSVLVLLPVEVATHYLEIITQLIVESFHFKNGEDAPDLLKVITKP |
| | | FTKLIVQLDKKVISQIAMNDEKAKNKSLVKIWCKTFTNKTQINVT |
| | | VPSTANCTSPSLCWTDGIQNWTMKNVTYKENIAKCQHIFVNFHLP |
| | | DLAVGTILLILSLLVLCGCLIMIVKILGSVLKGQVATVIKKTINT |
| | | DFPFPFAWLTGYLAILVGAGMTFIVQSSSVFTSALTPLIGIGVIT |
| | | IERAYPLTLGSNIGTTTTAILAALASPGNALRSSLQIALCHEFEN |
| | | ISGILLWYPIPFTRLPIRMAKGLGNISAKYRWFAVFYLIIFFFLI |
| | | PLTVFGLSLAGWRVLVGVGVPVVFIIILVLCLRLLQSRCPRVLPK |
| | | KLQNWNFLPLWMRSLKPWDAVVSKFTGCFQMRCCCCCRVCCRACC |
| | | LLCDCPKCCRCSKCCEDLEEAQEGQDVPVKAPETFDNITISREAQ |
| | | GEVPASDSKTECTAL |
| NECTIN4 | 135 | MPLSLGAEMWGPEAWLLLLLLLASFTGRCPAGELETSDVVTVVLG |
| (NP_112178.2) | | QDAKLPCFYRGDSGEQVGQVAWARVDAGEGAQELALLHSKYGLHV |
| | | SPAYEGRVEQPPPPRNPLDGSVLLRNAVQADEGEYECRVSTFPAG |
| | | SFQARLRLRVLVPPLPSLNPGPALEEGQGLTLAASCTAEGSPAPS |
| | | VTWDTEVKGTTSSRSFKHSRSAAVTSEFHLVPSRSMNGQPLTCVV |
| | | SHPGLLQDQRITHILHVSFLAEASVRGLEDQNLWHIGREGAMLKC |
| | | LSEGQPPPSYNWTRLDGPLPSGVRVDGDTLGFPPLTTEHSGIYVC |
| | | HVSNEFSSRDSQVTVDVLDPQEDSGKQVDLVSASVVVVGVIAALL |
| | | FCLLVVVVVLMSRYHRRKAQQMTQKYEEELTLTRENSIRRLHSHH |
| | | TDPRSQPEESVGLRAEGHPDSLKDNSSCSVMSEEPEGRSYSTLTT |
| | | VREIETQTELLSPGSGRAEEEEDQDEGIKQAMNHFVQENGTLRAK |
| | | PTGNGIYINGRGHLV |
| TACSTD2 | 136 | MARGPGLAPPPLRLPLLLLVLAAVTGHTAAQDNCTCPTNKMTVCS |
| (NP_002344.2) | | PDGPGGRCQCRALGSGMAVDCSTLTSKCLLLKARMSAPKNARTLV |
| | | RPSEHALVDNDGLYDPDCDPEGRFKARQCNQTSVCWCVNSVGVRR |
| | | TDKGDLSLRCDELVRTHHILIDLRHRPTAGAFNHSDLDAELRRLF |
| | | RERYRLHPKFVAAVHYEQPTIQIELRQNTSQKAAGDVDIGDAAYY |
| | | FERDIKGESLFQGRGGLDLRVRGEPLQVERTLIYYLDEIPPKFSM |
| | | KRLTAGLIAVIVVVVVALVAGMAVLVITNRRKSGKYKKVEIKELG |
| | | ELRKEPSL |
| SLC39A6 | 137 | MARKLSVILILTFALSVTNPLHELKAAAFPQTTEKISPNWESGIN |
| (NP_036451.4) | | VDLAISTRQYHLQQLFYRYGENNSLSVEGFRKLLQNIGIDKIKRI |
| | | HIHHDHDHHSDHEHHSDHERHSDHEHHSEHEHHSDHDHHSHHNHA |
| | | ASGKNKRKALCPDHDSDSSGKDPRNSQGKGAHRPEHASGRRNVKD |
| | | SVSASEVTSTVYNTVSEGTHFLETIETPRPGKLFPKDVSSSTPPS |
| | | VTSKSRVSRLAGRKTNESVSEPRKGFMYSRNTNENPQECFNASKL |
| | | LTSHGMGIQVPLNATEFNYLCPAIINQIDARSCLIHTSEKKAEIP |
| | | PKTYSLQIAWVGGFIAISIISFLSLLGVILVPLMNRVFFKFLLSF |
| | | LVALAVGTLSGDAFLHLLPHSHASHHHSHSHEEPAMEMKRGPLFS |
| | | HLSSQNIEESAYFDSTWKGLTALGGLYFMFLVEHVLTLIKQFKDK |
| | | KKKNQKKPENDDDVEIKKQLSKYESQLSTNEEKVDTDDRTEGYLR |
| | | ADSQEPSHEDSQQPAVLEEEEVMIAHAHPQEVYNEYVPRGCKNKC |
| | | HSHFHDTLGQSDDLIHHHHDYHHILHHHHHQNHHPHSHSQRYSRE |
| | | ELKDAGVATLAWMVIMGDGLHNFSDGLAIGAAFTEGLSSGLSTSV |
| | | AVFCHELPHELGDFAVLLKAGMTVKQAVLYNALSAMLAYLGMATG |
| | | IFIGHYAENVSMWIFALTAGLFMYVALVDMVPEMLHNDASDHGCS |
| | | RWGYFFLQNAGMLLGFGIMLLISIFEHKIVFRINF |
| GPNMB | 138 | MECLYYFLGFLLLAARLPLDAAKRFHDVLGNERPSAYMREHNQLN |
| (NP_001005340.1) | | GWSSDENDWNEKLYPVWKRGDMRWKNSWKGGRVQAVLTSDSPALV |
| | | GSNITFAVNLIFPRCQKEDANGNIVYEKNCRNEAGLSADPYVYNW |
| | | TAWSEDSDGENGTGQSHHNVFPDGKPFPHHPGWRRWNFIYVFHTL |
| | | GQYFQKLGRCSVRVSVNTANVTLGPQLMEVTVYRRHGRAYVPIAQ |
| | | VKDVYVVTDQIPVFVTMFQKNDRNSSDETFLKDLPIMFDVLIHDP |
| | | SHFLNYSTINYKWSFGDNTGLFVSTNHTVNHTYVLNGTFSLNLTV |
| | | KAAAPGPCPPPPPPPRPSKPTPSLATTLKSYDSNTPGPAGDNPLE |
| | | LSRIPDENCQINRYGHFQATITIVEGILEVNIIQMTDVLMPVPWP |
| | | ESSLIDFVVTCQGSIPTEVCTIISDPTCEITQNTVCSPVDVDEMC |
| | | LLTVRRTFNGSGTYCVNLTLGDDTSLALISTLISVPDRDPASPLR |
| | | MANSALISVGCLAIFVTVISLLVYKKHKEYNPIENSPGNVVRSKG |
| | | LSVFLNRAKAVFFPGNQEKDPLLKNQEFKGVS |
| MSLN | 139 | MALPTARPLLGSCGTPALGSLLFLLFSLGWVQPSRTLAGETGQEA |
| (NP_005814.2) | | APLDGVLANPPNISSLSPRQLLGFPCAEVSGLSTERVRELAVALA |
| | | QKNVKLSTEQLRCLAHRLSEPPEDLDALPLDLLLFLNPDAFSGPQ |
| | | ACTRFFSRITKANVDLLPRGAPERQRLLPAALACWGVRGSLLSEA |
| | | DVRALGGLACDLPGRFVAESAEVLLPRLVSCPGPLDQDQQEAARA |
| | | ALQGGGPPYGPPSTWSVSTMDALRGLLPVLGQPIIRSIPQGIVAA |
| | | WRQRSSRDPSWRQPERTILRPRFRREVEKTACPSGKKAREIDESL |
| | | IFYKKWELEACVDAALLATQMDRVNAIPFTYEQLDVLKHKLDELY |
| | | PQGYPESVIQHLGYLFLKMSPEDIRKWNVTSLETLKALLEVNKGH |
| | | EMSPQVATLIDRFVKGRGQLDKDTLDTLTAFYPGYLCSLSPEELS |
| | | SVPPSSIWAVRPQDLDTCDPRQLDVLYPKARLAFQNMNGSEYFVK |
| | | IQSFLGGAPTEDLKALSQQNVSMDLATFMKLRTDAVLPLTVAEVQ |
| | | KLLGPHVEGLKAEERHRPVRDWILRQRQDDLDTLGLGLQGGIPNG |
| | | YLVLDLSMQEALSGTPCLLGPGPVLTVLALLLASTLA |
| CD74 | 140 | MHRRRSRSCREDQKPVMDDQRDLISNNEQLPMLGRRPGAPESKCS |
| (NP_001020330.1) | | RGALYTGFSILVTLLLAGQATTAYFLYQQQGRLDKLTVTSQNLQL |
| | | ENLRMKLPKPPKPVSKMRMATPLLMQALPMGALPQGPMQNATKYG |
| | | NMTEDHVMHLLQNADPLKVYPPLKGSFPENLRHLKNTMETIDWKV |
| | | FESWMHHWLLFEMSRHSLEQKPTDAPPKVLTKCQEEVSHIPAVHP |
| | | GSFRPKCDENGNYLPLQCYGSIGYCWCVFPNGTEVPNTRSRGHHN |
| | | CSESLELEDPSSGLGVTKQDLGPVPM |
| F3 | 141 | METPAWPRVPRPETAVARTLLLGWVFAQVAGASGTTNTVAAYNLT |
| (NP_001984.1) | | WKSTNFKTILEWEPKPVNQVYTVQISTKSGDWKSKCFYTTDTECD |
| | | LTDEIVKDVKQTYLARVFSYPAGNVESTGSAGEPLYENSPEFTPY |
| | | LETNLGQPTIQSFEQVGTKVNVTVEDERTLVRRNNTFLSLRDVFG |
| | | KDLIYTLYYWKSSSSGKKTAKTNTNEFLIDVDKGENYCFSVQAVI |
| | | PSRTVNRKSTDSPVECMGQEKGEFREIFYIIGAVVFVVIILVIIL |
| | | AISLHKCRKAGVGQSWKENSPLNVS |
| MUC16 | 142 | MLKPSGLPGSSSPTRSLMTGSRSTKATPEMDSGLTGATLSPKTST |
| (NP_078966.2) | | GAIVVTEHTLPFTSPDKTLASPTSSVVGRTTQSLGVMSSALPEST |
| | | SRGMTHSEQRTSPSLSPQVNGTPSRNYPATSMVSGLSSPRTRTSS |
| | | TEGNFTKEASTYTLTVETTSGPVTEKYTVPTETSTTEGDSTETPW |
| | | DTRYIPVKITSPMKTFADSTASKENAPVSMTPAETTVTDSHTPGR |
| | | TNPSFGTLYSSFLDLSPKGTPNSRGETSLELILSTTGYPFSSPEP |
| | | GSAGHSRISTSAPLSSSASVLDNKISETSIFSGQSLTSPLSPGVP |
| | | EARASTMPNSAIPFSMTLSNAETSAERVRSTISSLGTPSISTKQT |
| | | AETILTFHAFAETMDIPSTHIAKTLASEWLGSPGTLGGTSTSALT |
| | | TTSPSTTLVSEETNTHHSTSGKETEGTLNTSMTPLETSAPGEESE |
| | | MTATLVPTLGFTTLDSKIRSPSQVSSSHPTRELRTTGSTSGRQSS |
| | | STAAHGSSDILRATTSSTSKASSWTSESTAQQFSEPQHTQWVETS |
| | | PSMKTERPPASTSVAAPITTSVPSVVSGFTTLKTSSTKGIWLEET |
| | | SADTLIGESTAGPTTHQFAVPTGISMTGGSSTRGSQGTTHLLTRA |
| | | TASSETSADLTLATNGVPVSVSPAVSKTAAGSSPPGGTKPSYTMV |
| | | SSVIPETSSLQSSAFREGTSLGLTPLNTRHPFSSPEPDSAGHTKI |
| | | STSIPLLSSASVLEDKVSATSTFSHHKATSSITTGTPEISTKTKP |
| | | SSAVLSSMTLSNAATSPERVRNATSPLTHPSPSGEETAGSVLTLS |
| | | TSAETTDSPNIHPTGTLTSESSESPSTLSLPSVSGVKTTFSSSTP |
| | | STHLFTSGEETEETSNPSVSQPETSVSRVRTTLASTSVPTPVFPT |
| | | MDTWPTRSAQFSSSHLVSELRATSSTSVINSTGSALPKISHLTGT |
| | | ATMSQTNRDTENDSAAPQSTTWPETSPRFKTGLPSATTTVSTSAT |
| | | SLSATVMVSKFTSPATSSMEATSIREPSTTILTTETTNGPGSMAV |
| | | ASTNIPIGKGYITEGRLDTSHLPIGTTASSETSMDFTMAKESVSM |
| | | SVSPSQSMDAAGSSTPGRTSQFVDTFSDDVYHLTSREITIPRDGT |
| | | SSALTPQMTATHPPSPDPGSARSTWLGILSSSPSSPTPKVTMSST |
| | | FSTQRVTTSMIMDTVETSRWNMPNLPSTTSLTPSNIPTSGAIGKS |
| | | TLVPLDTPSPATSLEASEGGLPTLSTYPESTNTPSIHLGAHASSE |
| | | SPSTIKLTMASVVKPGSYTPLTFPSIETHIHVSTARMAYSSGSSP |
| | | EMTAPGETNTGSTWDPTTYITTTDPKDTSSAQVSTPHSVRTLRTT |
| | | ENHPKTESATPAAYSGSPKISSSPNLTSPATKAWTITDTTEHSTQ |
| | | LHYTKLAEKSSGFETQSAPGPVSVVIPTSPTIGSSTLELTSDVPG |
| | | EPLVLAPSEQTTITLPMATWLSTSLTEEMASTDLDISSPSSPMST |
| | | FAIFPPMSTPSHELSKSEADTSAIRNTDSTTLDQHLGIRSLGRTG |
| | | DLTTVPITPLTTTWTSVIEHSTQAQDTLSATMSPTHVTQSLKDQT |
| | | SIPASASPSHLTEVYPELGTQGRSSSEATTFWKPSTDTLSREIET |
| | | GPTNIQSTPPMDNTTTGSSSSGVTLGIAHLPIGTSSPAETSTNMA |
| | | LERRSSTATVSMAGTMGLLVTSAPGRSISQSLGRVSSVLSESTTE |
| | | GVTDSSKGSSPRLNTQGNTALSSSLEPSYAEGSQMSTSIPLTSSP |
| | | TTPDVEFIGGSTFWTKEVTTVMTSDISKSSARTESSSATLMSTAL |
| | | GSTENTGKEKLRTASMDLPSPTPSMEVTPWISLTLSNAPNTTDSL |
| | | DLSHGVHTSSAGTLATDRSLNTGVTRASRLENGSDTSSKSLSMGN |
| | | STHTSMTYTEKSEVSSSIHPRPETSAPGAETTLTSTPGNRAISLT |
| | | LPFSSIPVEEVISTGITSGPDINSAPMTHSPITPPTIVWTSTGTI |
| | | EQSTQPLHAVSSEKVSVQTQSTPYVNSVAVSASPTHENSVSSGSS |
| | | TSSPYSSASLESLDSTISRRNAITSWLWDLTTSLPTTTWPSTSLS |
| | | EALSSGHSGVSNPSSTTTEFPLFSAASTSAAKQRNPETETHGPQN |
| | | TAASTLNTDASSVTGLSETPVGASISSEVPLPMAITSRSDVSGLT |
| | | SESTANPSLGTASSAGTKLTRTISLPTSESLVSFRMNKDPWTVSI |
| | | PLGSHPTTNTETSIPVNSAGPPGLSTVASDVIDTPSDGAESIPTV |
| | | SFSPSPDTEVTTISHFPEKTTHSFRTISSLTHELTSRVTPIPGDW |
| | | MSSAMSTKPTGASPSITLGERRTITSAAPTTSPIVLTASFTETST |
| | | VSLDNETTVKTSDILDARKTNELPSDSSSSSDLINTSIASSTMDV |
| | | TKTASISPTSISGMTASSSPSLFSSDRPQVPTSTTETNTATSPSV |
| | | SSNTYSLDGGSNVGGTPSTLPPFTITHPVETSSALLAWSRPVRTF |
| | | STMVSTDTASGENPTSSNSVVTSVPAPGTWTSVGSTTDLPAMGFL |
| | | KTSPAGEAHSLLASTIEPATAFTPHLSAAVVTGSSATSEASLLTT |
| | | SESKAIHSSPQTPTTPTSGANWETSATPESLLVVTETSDTTLTSK |
| | | ILVTDTILFSTVSTPPSKFPSTGTLSGASFPTLLPDTPAIPLTAT |
| | | EPTSSLATSFDSTPLVTIASDSLGTVPETTLTMSETSNGDALVLK |
| | | TVSNPDRSIPGITIQGVTESPLHPSSTSPSKIVAPRNTTYEGSIT |
| | | VALSTLPAGTTGSLVFSQSSENSETTALVDSSAGLERASVMPLTT |
| | | GSQGMASSGGIRSGSTHSTGTKTFSSLPLTMNPGEVTAMSEITTN |
| | | RLTATQSTAPKGIPVKPTSAESGLLTPVSASSSPSKAFASLTTAP |
| | | PTWGIPQSTLTFEFSEVPSLDTKSASLPTPGQSLNTIPDSDASTA |
| | | SSSLSKSPEKNPRARMMTSTKAISASSFQSTGFTETPEGSASPSM |
| | | AGHEPRVPTSGTGDPRYASESMSYPDPSKASSAMTSTSLASKLTT |
| | | LFSTGQAARSGSSSSPISLSTEKETSFLSPTASTSRKTSLFLGPS |
| | | MARQPNILVHLQTSALTLSPTSTLNMSQEEPPELTSSQTIAEEEG |
| | | TTAETQTLTFTPSETPTSLLPVSSPTEPTARRKSSPETWASSISV |
| | | PAKTSLVETTDGTLVTTIKMSSQAAQGNSTWPAPAEETGSSPAGT |
| | | SPGSPEMSTTLKIMSSKEPSISPEIRSTVRNSPWKTPETTVPMET |
| | | TVEPVTLQSTALGSGSTSISHLPTGTTSPTKSPTENMLATERVSL |
| | | SPSPPEAWTNLYSGTPGGTRQSLATMSSVSLESPTARSITGTGQQ |
| | | SSPELVSKTTGMEFSMWHGSTGGTTGDTHVSLSTSSNILEDPVTS |
| | | PNSVSSLTDKSKHKTETWVSTTAIPSTVLNNKIMAAEQQTSRSVD |
| | | EAYSSTSSWSDQTSGSDITLGASPDVTNTLYITSTAQTTSLVSLP |
| | | SGDQGITSLTNPSGGKTSSASSVTSPSIGLETLRANVSAVKSDIA |
| | | PTAGHLSQTSSPAEVSILDVTTAPTPGISTTITTMGINSISTTTP |
| | | NPEVGMSTMDSTPATERRTTSTEHPSTWSSTAASDSWTVTDMTSN |
| | | LKVARSPGTISTMHTTSFLASSTELDSMSTPHGRITVIGTSLVTP |
| | | SSDASAVKTETSTSERTLSPSDTTASTPISTFSRVQRMSISVPDI |
| | | LSTSWTPSSTEAEDVPVSMVSTDHASTKTDPNTPLSTFLFDSLST |
| | | LDWDTGRSLSSATATTSAPQGATTPQELTLETMISPATSQLPFSI |
| | | GHITSAVTPAAMARSSGVTFSRPDPTSKKAEQTSTQLPTTTSAHP |
| | | GQVPRSAATTLDVIPHTAKTPDATFQRQGQTALTTEARATSDSWN |
| | | EKEKSTPSAPWITEMMNSVSEDTIKEVTSSSSVLRTLNTLDINLE |
| | | SGTTSSPSWKSSPYERIAPSESTTDKEAIHPSTNTVETTGWVTSS |
| | | EHASHSTIPAHSASSKLTSPVVTTSTREQAIVSMSTTTWPESTRA |
| | | RTEPNSFLTIELRDVSPYMDTSSTTQTSIISSPGSTAITKGPRTE |
| | | ITSSKRISSSFLAQSMRSSDSPSEAITRLSNFPAMTESGGMILAM |
| | | QTSPPGATSLSAPTLDTSATASWTGTPLATTQRFTYSEKTTLFSK |
| | | GPEDTSQPSPPSVEETSSSSSLVPIHATTSPSNILLTSQGHSPSS |
| | | TPPVTSVFLSETSGLGKTTDMSRISLEPGTSLPPNLSSTAGEALS |
| | | TYEASRDTKAIHHSADTAVTNMEATSSEYSPIPGHTKPSKATSPL |
| | | VTSHIMGDITSSTSVFGSSETTEIETVSSVNQGLQERSTSQVASS |
| | | ATETSTVITHVSSGDATTHVTKTQATFSSGTSISSPHQFITSTNT |
| | | FTDVSTNPSTSLIMTESSGVTITTQTGPTGAATQGPYLLDTSTMP |
| | | YLTETPLAVTPDFMQSEKTTLISKGPKDVSWTSPPSVAETSYPSS |
| | | LTPFLVTTIPPATSTLQGQHTSSPVSATSVLTSGLVKTTDMLNTS |
| | | MEPVTNSPQNLNNPSNEILATLAATTDIETIHPSINKAVTNMGTA |
| | | SSAHVLHSTLPVSSEPSTATSPMVPASSMGDALASISIPGSETTD |
| | | IEGEPTSSLTAGRKENSTLQEMNSTTESNIILSNVSVGAITEATK |
| | | MEVPSFDATFIPTPAQSTKFPDIFSVASSRLSNSPPMTISTHMTT |
| | | TQTGSSGATSKIPLALDTSTLETSAGTPSVVTEGFAHSKITTAMN |
| | | NDVKDVSQTNPPFQDEASSPSSQAPVLVTTLPSSVAFTPQWHSTS |
| | | SPVSMSSVLTSSLVKTAGKVDTSLETVTSSPQSMSNTLDDISVTS |
| | | AATTDIETTHPSINTVVTNVGTTGSAFESHSTVSAYPEPSKVTSP |
| | | NVTTSTMEDTTISRSIPKSSKTTRTETETTSSLTPKLRETSISQE |
| | | ITSSTETSTVPYKELTGATTEVSRTDVTSSSSTSFPGPDQSTVSL |
| | | DISTETNTRLSTSPIMTESAEITITTQTGPHGATSQDTFTMDPSN |
| | | TTPQAGIHSAMTHGFSQLDVTTLMSRIPQDVSWTSPPSVDKTSSP |
| | | SSFLSSPAMTTPSLISSTLPEDKLSSPMTSLLTSGLVKITDILRT |
| | | RLEPVTSSLPNFSSTSDKILATSKDSKDTKEIFPSINTEETNVKA |
| | | NNSGHESHSPALADSETPKATTQMVITTTVGDPAPSTSMPVHGSS |
| | | ETTNIKREPTYFLTPRLRETSTSQESSFPTDTSFLLSKVPTGTIT |
| | | EVSSTGVNSSSKISTPDHDKSTVPPDTFTGEIPRVFTSSIKTKSA |
| | | EMTITTQASPPESASHSTLPLDTSTTLSQGGTHSTVTQGFPYSEV |
| | | TTLMGMGPGNVSWMTTPPVEETSSVSSLMSSPAMTSPSPVSSTSP |
| | | QSIPSSPLPVTALPTSVLVTTTDVLGTTSPESVTSSPPNLSSITH |
| | | ERPATYKDTAHTEAAMHHSTNTAVTNVGTSGSGHKSQSSVLADSE |
| | | TSKATPLMSTTSTLGDTSVSTSTPNISQTNQIQTEPTASLSPRLR |
| | | ESSTSEKTSSTTETNTAFSYVPTGAITQASRTEISSSRTSISDLD |
| | | RPTIAPDISTGMITRLFTSPIMTKSAEMTVTTQTTTPGATSQGIL |
| | | PWDTSTTLFQGGTHSTVSQGFPHSEITTLRSRTPGDVSWMTTPPV |
| | | EETSSGFSLMSPSMTSPSPVSSTSPESIPSSPLPVTALLTSVLVT |
| | | TTNVLGTTSPEPVTSSPPNLSSPTQERLTTYKDTAHTEAMHASMH |
| | | TNTAVANVGTSISGHESQSSVPADSHTSKATSPMGITFAMGDTSV |
| | | STSTPAFFETRIQTESTSSLIPGLRDTRTSEEINTVTETSTVLSE |
| | | VPTTTTTEVSRTEVITSSRTTISGPDHSKMSPYISTETITRLSTF |
| | | PFVTGSTEMAITNQTGPIGTISQATLTLDTSSTASWEGTHSPVTQ |
| | | RFPHSEETTTMSRSTKGVSWQSPPSVEETSSPSSPVPLPAITSHS |
| | | SLYSAVSGSSPTSALPVTSLLTSGRRKTIDMLDTHSELVTSSLPS |
| | | ASSFSGEILTSEASTNTETIHFSENTAETNMGTTNSMHKLHSSVS |
| | | IHSQPSGHTPPKVTGSMMEDAIVSTSTPGSPETKNVDRDSTSPLT |
| | | PELKEDSTALVMNSTTESNTVFSSVSLDAATEVSRAEVTYYDPTF |
| | | MPASAQSTKSPDISPEASSSHSNSPPLTISTHKTIATQTGPSGVT |
| | | SLGQLTLDTSTIATSAGTPSARTQDFVDSETTSVMNNDLNDVLKT |
| | | SPFSAEEANSLSSQAPLLVTTSPSPVTSTLQEHSTSSLVSVTSVP |
| | | TPTLAKITDMDTNLEPVTRSPQNLRNTLATSEATTDTHTMHPSIN |
| | | TAVANVGTTSSPNEFYFTVSPDSDPYKATSAVVITSTSGDSIVST |
| | | SMPRSSAMKKIESETTFSLIFRLRETSTSQKIGSSSDTSTVEDKA |
| | | FTAATTEVSRTELTSSSRTSIQGTEKPTMSPDTSTRSVTMLSTFA |
| | | GLTKSEERTIATQTGPHRATSQGTLTWDTSITTSQAGTHSAMTHG |
| | | FSQLDLSTLTSRVPEYISGTSPPSVEKTSSSSSLLSLPAITSPSP |
| | | VPTTLPESRPSSPVHLTSLPTSGLVKTTDMLASVASLPPNLGSTS |
| | | HKIPTTSEDIKDTEKMYPSTNIAVTNVGTTTSEKESYSSVPAYSE |
| | | PPKVTSPMVTSFNIRDTIVSTSMPGSSEITRIEMESTFSLAHGLK |
| | | GTSTSQDPIVSTEKSAVLHKLTTGATETSRTEVASSRRTSIPGPD |
| | | HSTESPDISTEVIPSLPISLGITESSNMTIITRTGPPLGSTSQGT |
| | | FTLDTPTTSSRAGTHSMATQEFPHSEMTTVMNKDPEILSWTIPPS |
| | | IEKTSFSSSLMPSPAMTSPPVSSTLPKTIHTTPSPMTSLLTPSLV |
| | | MTTDTLGTSPEPTTSSPPNLSSTSHEILTTDEDTTAIEAMHPSTS |
| | | TAATNVETTSSGHGSQSSVLADSEKTKATAPMDTTSTMGHTTVST |
| | | SMSVSSETTKIKRESTYSLIPGLRETSISQNASFSTDTSIVLSEV |
| | | PTGTTAEVSRTEVTSSGRTSIPGPSQSTVLPEISTRTMTRLFASP |
| | | TMTESAEMTIPTQTGPSGSTSQDTLTLDTSTTKSQAKTHSTLTQR |
| | | FPHSEMTTLMSRGPGDMSWQSSPSLENPSSLPSLLSLPATTSPPP |
| | | ISSTLPVTISSSPLPVTSLLTSSPVTTTDMLHTSPELVTSSPPKL |
| | | SHTSDERLTTGKDTINTEAVHPSTNTAASNVEIPSSGHESPSSAL |
| | | ADSETSKATSPMFITSTQEDTTVAISTPHFLETSRIQKESISSLS |
| | | PKLRETGSSVETSSAIETSAVLSEVSIGATTEISRTEVTSSSRTS |
| | | ISGSAESTMLPEISTTRKIIKFPTSPILAESSEMTIKTQTSPPGS |
| | | TSESTFTLDTSTTPSLVITHSTMTQRLPHSEITTLVSRGAGDVPR |
| | | PSSLPVEETSPPSSQLSLSAMISPSPVSSTLPASSHSSSASVTSL |
| | | LTPGQVKTTEVLDASAEPETSSPPSLSSTSVEILATSEVTTDTEK |
| | | IHPFSNTAVTKVGTSSSGHESPSSVLPDSETTKATSAMGTISIMG |
| | | DTSVSTLTPALSNTRKIQSEPASSLTTRLRETSTSEETSLATEAN |
| | | TVLSKVSTGATTEVSRTEAISFSRTSMSGPEQSTMSQDISIGTIP |
| | | RISASSVLTESAKMTITTQTGPSESTLESTLNLNTATTPSWVETH |
| | | SIVIQGFPHPEMTTSMGRGPGGVSWPSPPFVKETSPPSSPLSLPA |
| | | VTSPHPVSTTFLAHIPPSPLPVTSLLTSGPATTTDILGTSTEPGT |
| | | SSSSSLSTTSHERLTTYKDTAHTEAVHPSTNTGGTNVATTSSGYK |
| | | SQSSVLADSSPMCTTSTMGDTSVLTSTPAFLETRRIQTELASSLT |
| | | PGLRESSGSEGTSSGTKMSTVLSKVPTGATTEISKEDVTSIPGPA |
| | | QSTISPDISTRTVSWFSTSPVMTESAEITMNTHTSPLGATTQGTS |
| | | TLDTSSTTSLTMTHSTISQGFSHSQMSTLMRRGPEDVSWMSPPLL |
| | | EKTRPSFSLMSSPATTSPSPVSSTLPESISSSPLPVTSLLTSGLA |
| | | KTTDMLHKSSEPVTNSPANLSSTSVEILATSEVTTDTEKTHPSSN |
| | | RTVTDVGTSSSGHESTSFVLADSQTSKVTSPMVITSTMEDTSVST |
| | | STPGFFETSRIQTEPTSSLTLGLRKTSSSEGTSLATEMSTVLSGV |
| | | PTGATAEVSRTEVTSSSRTSISGFAQLTVSPETSTETITRLPTSS |
| | | IMTESAEMMIKTQTDPPGSTPESTHTVDISTTPNWVETHSTVTQR |
| | | FSHSEMTTLVSRSPGDMLWPSQSSVEETSSASSLLSLPATTSPSP |
| | | VSSTLVEDFPSASLPVTSLLNPGLVITTDRMGISREPGTSSTSNL |
| | | SSTSHERLTTLEDTVDTEDMQPSTHTAVINVRTSISGHESQSSVL |
| | | SDSETPKATSPMGTTYTMGETSVSISTSDFFETSRIQIEPTSSLT |
| | | SGLRETSSSERISSATEGSTVLSEVPSGATTEVSRTEVISSRGTS |
| | | MSGPDQFTISPDISTEAITRLSTSPIMTESAESAITIETGSPGAT |
| | | SEGTLTLDTSTTTFWSGTHSTASPGFSHSEMTTLMSRTPGDVPWP |
| | | SLPSVEEASSVSSSLSSPAMTSTSFFSTLPESISSSPHPVTALLT |
| | | LGPVKTTDMLRTSSEPETSSPPNLSSTSAEILATSEVTKDREKIH |
| | | PSSNTPVVNVGTVIYKHLSPSSVLADLVTTKPTSPMATTSTLGNT |
| | | SVSTSTPAFPETMMTQPTSSLTSGLREISTSQETSSATERSASLS |
| | | GMPTGATTKVSRTEALSLGRTSTPGPAQSTISPEISTETITRIST |
| | | PLTTTGSAEMTITPKTGHSGASSQGTFTLDTSSRASWPGTHSAAT |
| | | HRSPHSGMTTPMSRGPEDVSWPSRPSVEKTSPPSSLVSLSAVTSP |
| | | SPLYSTPSESSHSSPLRVTSLFTPVMMKTTDMLDTSLEPVTTSPP |
| | | SMNITSDESLATSKATMETEAIQLSENTAVTQMGTISARQEFYSS |
| | | YPGLPEPSKVTSPVVTSSTIKDIVSTTIPASSEITRIEMESTSTL |
| | | TPTPRETSTSQEIHSATKPSTVPYKALTSATIEDSMTQVMSSSRG |
| | | PSPDQSTMSQDISTEVITRLSTSPIKTESTEMTITTQTGSPGATS |
| | | RGTLTLDTSTTFMSGTHSTASQGFSHSQMTALMSRTPGDVPWLSH |
| | | PSVEEASSASFSLSSPVMTSSSPVSSTLPDSIHSSSLPVTSLLTS |
| | | GLVKTTELLGTSSEPETSSPPNLSSTSAEILAITEVTTDTEKLEM |
| | | TNVVTSGYTHESPSSVLADSVTTKATSSMGITYPTGDTNVLTSTP |
| | | AFSDTSRIQTKSKLSLTPGLMETSISEETSSATEKSTVLSSVPTG |
| | | ATTEVSRTEAISSSRTSIPGPAQSTMSSDTSMETITRISTPLTRK |
| | | ESTDMAITPKTGPSGATSQGTFTLDSSSTASWPGTHSATTQRFPQ |
| | | SVVTTPMSRGPEDVSWPSPLSVEKNSPPSSLVSSSSVTSPSPLYS |
| | | TPSGSSHSSPVPVTSLFTSIMMKATDMLDASLEPETTSAPNMNIT |
| | | SDESLAASKATTETEAIHVFENTAASHVETTSATEELYSSSPGES |
| | | EPTKVISPVVTSSSIRDNMVSTTMPGSSGITRIEIESMSSLTPGL |
| | | RETRTSQDITSSTETSTVLYKMPSGATPEVSRTEVMPSSRTSIPG |
| | | PAQSTMSLDISDEVVTRLSTSPIMTESAEITITTQTGYSLATSQV |
| | | TLPLGTSMTFLSGTHSTMSQGLSHSEMTNLMSRGPESLSWTSPRF |
| | | VETTRSSSSLTSLPLTTSLSPVSSTLLDSSPSSPLPVTSLILPGL |
| | | VKTTEVLDTSSEPKTSSSPNLSSTSVEIPATSEIMTDTEKIHPSS |
| | | NTAVAKVRTSSSVHESHSSVLADSETTITIPSMGITSAVDDTTVF |
| | | TSNPAFSETRRIPTEPTFSLTPGFRETSTSEETTSITETSAVLYG |
| | | VPTSATTEVSMTEIMSSNRIHIPDSDQSTMSPDIITEVITRLSSS |
| | | SMMSESTQMTITTQKSSPGATAQSTLTLATTTAPLARTHSTVPPR |
| | | FLHSEMTTLMSRSPENPSWKSSLFVEKTSSSSSLLSLPVTTSPSV |
| | | SSTLPQSIPSSSFSVTSLLTPGMVKTTDTSTEPGTSLSPNLSGTS |
| | | VEILAASEVTTDTEKIHPSSSMAVINVGTTSSGHELYSSVSIHSE |
| | | PSKATYPVGTPSSMAETSISTSMPANFETTGFEAEPFSHLTSGFR |
| | | KTNMSLDTSSVTPTNTPSSPGSTHLLQSSKTDFTSSAKTSSPDWP |
| | | PASQYTEIPVDIITPFNASPSITESTGITSFPESRFTMSVTESTH |
| | | HLSTDLLPSAETISTGTVMPSLSEAMTSFATTGVPRAISGSGSPF |
| | | SRTESGPGDATLSTIAESLPSSTPVPFSSSTFTTTDSSTIPALHE |
| | | ITSSSATPYRVDTSLGTESSTTEGRLVMVSTLDTSSQPGRTSSSP |
| | | ILDTRMTESVELGTVTSAYQVPSLSTRLTRTDGIMEHITKIPNEA |
| | | AHRGTIRPVKGPQTSTSPASPKGLHTGGTKRMETTTTALKTTTTA |
| | | LKTTSRATLTTSVYTPTLGTLTPLNASMQMASTIPTEMMITTPYV |
| | | FPDVPETTSSLATSLGAETSTALPRTTPSVFNRESETTASLVSRS |
| | | GAERSPVIQTLDVSSSEPDTTASWVIHPAETIPTVSKTTPNFFHS |
| | | ELDTVSSTATSHGADVSSAIPTNISPSELDALTPLVTISGTDTST |
| | | TFPTLTKSPHETETRTTWLTHPAETSSTIPRTIPNFSHHESDATP |
| | | SIATSPGAETSSAIPIMTVSPGAEDLVTSQVTSSGTDRNMTIPTL |
| | | TLSPGEPKTIASLVTHPEAQTSSAIPTSTISPAVSRLVTSMVTSL |
| | | AAKTSTTNRALTNSPGEPATTVSLVTHPAQTSPTVPWTTSIFFHS |
| | | KSDTTPSMTTSHGAESSSAVPTPTVSTEVPGVVTPLVTSSRAVIS |
| | | TTIPILTLSPGEPETTPSMATSHGEEASSAIPTPTVSPGVPGVVT |
| | | SLVTSSRAVTSTTIPILTFSLGEPETTPSMATSHGTEAGSAVPTV |
| | | LPEVPGMVTSLVASSRAVTSTTLPTLTLSPGEPETTPSMATSHGA |
| | | EASSTVPTVSPEVPGVVTSLVTSSSGVNSTSIPTLILSPGELETT |
| | | PSMATSHGAEASSAVPTPTVSPGVSGVVTPLVTSSRAVTSTTIPI |
| | | LTLSSSEPETTPSMATSHGVEASSAVLTVSPEVPGMVTSLVTSSR |
| | | AVTSTTIPTLTISSDEPETTTSLVTHSEAKMISAIPTLAVSPTVQ |
| | | GLVTSLVTSSGSETSAFSNITVASSQPETIDSWVAHPGTEASSVV |
| | | PTLTVSTGEPFTNISLVTHPAESSSTLPRTTSRFSHSELDTMPST |
| | | VTSPEAESSSAISTTISPGIPGVLTSLVTSSGRDISATFPTVPES |
| | | PHESEATASWVTHPAVTSTTVPRTTPNYSHSEPDTTPSIATSPGA |
| | | EATSDFPTITVSPDVPDMVTSQVTSSGTDTSITIPTLTLSSGEPE |
| | | TTTSFITYSETHTSSAIPTLPVSPGASKMLTSLVISSGTDSTTTF |
| | | PTLTETPYEPETTAIQLIHPAETNTMVPRTTPKFSHSKSDTTLPV |
| | | AITSPGPEASSAVSTTTISPDMSDLVTSLVPSSGTDTSTTFPTLS |
| | | ETPYEPETTATWLTHPAETSTTVSGTIPNFSHRGSDTAPSMVTSP |
| | | GVDTRSGVPTTTIPPSIPGVVTSQVTSSATDTSTAIPTLTPSPGE |
| | | PETTASSATHPGTQTGFTVPIRTVPSSEPDTMASWVTHPPQTSTP |
| | | VSRTTSSFSHSSPDATPVMATSPRTEASSAVLTTISPGAPEMVTS |
| | | QITSSGAATSTTVPTLTHSPGMPETTALLSTHPRTETSKTFPAST |
| | | VFPQVSETTASLTIRPGAETSTALPTQTTSSLFTLLVTGTSRVDL |
| | | SPTASPGVSAKTAPLSTHPGTETSTMIPTSTLSLGLLETTGLLAT |
| | | SSSAETSTSTLTLTVSPAVSGLSSASITTDKPQTVTSWNTETSPS |
| | | VTSVGPPEFSRTVTGTTMTLIPSEMPTPPKTSHGEGVSPTTILRT |
| | | TMVEATNLATTGSSPTVAKTTTTFNTLAGSLFTPLTTPGMSTLAS |
| | | ESVTSRTSYNHRSWISTTSSYNRRYWTPATSTPVTSTFSPGISTS |
| | | SIPSSTAATVPFMVPFTLNFTITNLQYEEDMRHPGSRKFNATERE |
| | | LQGLLKPLFRNSSLEYLYSGCRLASLRPEKDSSATAVDAICTHRP |
| | | DPEDLGLDRERLYWELSNLINGIQELGPYTLDRNSLYVNGFTHRS |
| | | SMPTTSTPGTSTVDVGTSGTPSSSPSPTTAGPLLMPFTLNFTITN |
| | | LQYEEDMRRTGSRKFNTMESVLQGLLKPLFKNTSVGPLYSGCRLT |
| | | LLRPEKDGAATGVDAICTHRLDPKSPGLNREQLYWELSKLINDIE |
| | | ELGPYTLDRNSLYVNGFTHQSSVSTTSTPGTSTVDLRTSGTPSSL |
| | | SSPTIMAAGPLLVPFTLNFTITNLQYGEDMGHPGSRKFNTTERVL |
| | | QGLLGPIFKNTSVGPLYSGCRLTSLRSEKDGAATGVDAICIHHLD |
| | | PKSPGLNRERLYWELSQLTNGIKELGPYTLDRNSLYVNGFTHRTS |
| | | VPTSSTPGTSTVDLGTSGTPFSLPSPATAGPLLVLFTLNFTITNL |
| | | KYEEDMHRPGSRKFNTTERVLQTLLGPMFKNTSVGLLYSGCRLTL |
| | | LRSEKDGAATGVDAICTHRLDPKSPGVDREQLYWELSQLTNGIKE |
| | | LGPYTLDRNSLYVNGFTHWIPVPTSSTPGTSTVDLGSGTPSSLPS |
| | | PTTAGPLLVPFTLNFTITNLKYEEDMHCPGSRKENTTERVLQSLL |
| | | GPMFKNTSVGPLYSGCRLTLLRSEKDGAATGVDAICTHRLDPKSP |
| | | GVDREQLYWELSQLTNGIKELGPYTLDRNSLYVNGFTHQTSAPNT |
| | | STPGTSTVDLGTSGTPSSLPSPTSAGPLLVPFTLNFTITNLQYEE |
| | | DMHHPGSRKFNTTERVLQGLLGPMFKNTSVGLLYSGCRLTLLRPE |
| | | KNGAATGMDAICSHRLDPKSPGLNREQLYWELSQLTHGIKELGPY |
| | | TLDRNSLYVNGFTHRSSVAPTSTPGTSTVDLGTSGTPSSLPSPTT |
| | | AVPLLVPFTLNFTITNLQYGEDMRHPGSRKFNTTERVLQGLLGPL |
| | | FKNSSVGPLYSGCRLISLRSEKDGAATGVDAICTHHLNPQSPGLD |
| | | REQLYWQLSQMTNGIKELGPYTLDRNSLYVNGFTHRSSGLITSTP |
| | | WTSTVDLGTSGTPSPVPSPTTTGPLLVPFTLNFTITNLQYEENMG |
| | | HPGSRKFNITESVLQGLLKPLFKSTSVGPLYSGCRLTLLRPEKDG |
| | | VATRVDAICTHRPDPKIPGLDRQQLYWELSQLTHSITELGPYTLD |
| | | RDSLYVNGFTQRSSVPTTSTPGTFTVQPETSETPSSLPGPTATGP |
| | | VLLPFTLNFTITNLQYEEDMRRPGSRKENTTERVLQGLLMPLFKN |
| | | TSVSSLYSGCRLTLLRPEKDGAATRVDAVCTHRPDPKSPGLDRER |
| | | LYWKLSQLTHGITELGPYTLDRHSLYVNGFTHQSSMTTTRTPDTS |
| | | TMHLATSRTPASLSGPMTASPLLVLFTINFTITNLRYEENMHHPG |
| | | SRKFNTTERVLQGLLRPVFKNTSVGPLYSGCRLTLLRPKKDGAAT |
| | | KVDAICTYRPDPKSPGLDREQLYWELSQLTHSITELGPYTLDRDS |
| | | LYVNGFTQRSSVPTTSIPGTPTVDLGTSGTPVSKPGPSAASPLLV |
| | | LFTLNFTITNLRYEENMQHPGSRKFNTTERVLQGLLRSLFKSTSV |
| | | GPLYSGCRLTLLRPEKDGTATGVDAICTHHPDPKSPRLDREQLYW |
| | | ELSQLTHNITELGPYALDNDSLFVNGFTHRSSVSTTSTPGTPTVY |
| | | LGASKTPASIFGPSAASHLLILFTLNFTITNLRYEENMWPGSRKF |
| | | NTTERVLQGLLRPLFKNTSVGPLYSGCRLTLLRPEKDGEATGVDA |
| | | ICTHRPDPTGPGLDREQLYLELSQLTHSITELGPYTLDRDSLYVN |
| | | GFTHRSSVPTTSTGVVSEEPFTLNFTINNLRYMADMGQPGSLKEN |
| | | ITDNVMQHLLSPLFQRSSLGARYTGCRVIALRSVKNGAETRVDLL |
| | | CTYLQPLSGPGLPIKQVFHELSQQTHGITRLGPYSLDKDSLYLNG |
| | | YNEPGPDEPPTTPKPATTFLPPLSEATTAMGYHLKTLTLNFTISN |
| | | LQYSPDMGKGSATFNSTEGVLQHLLRPLFQKSSMGPFYLGCQLIS |
| | | LRPEKDGAATGVDTTCTYHPDPVGPGLDIQQLYWELSQLTHGVTQ |
| | | LGFYVLDRDSLFINGYAPQNLSIRGEYQINFHIVNWNLSNPDPTS |
| | | SEYITLLRDIQDKVTTLYKGSQLHDTFRFCLVTNLTMDSVLVTVK |
| | | ALFSSNLDPSLVEQVELDKTLNASFHWLGSTYQLVDIHVTEMESS |
| | | VYQPTSSSSTQHFYLNFTITNLPYSQDKAQPGTTNYQRNKRNIED |
| | | ALNQLERNSSIKSYFSDCQVSTFRSVPNRHHTGVDSLCNFSPLAR |
| | | RVDRVAIYEEFLRMTRNGTQLQNFTLDRSSVLVDGYSPNRNEPLT |
| | | GNSDLPFWAVILIGLAGLLGVITCLICGVLVTTRRRKKEGEYNVQ |
| | | QQCPGYYQSHLDLEDLQ |
| SEZ6 | 231 | MRPVALLLLPSLLALLAHGLSLEAPTVGKGQAPGIEETDGELTAA |
| | | PTPEQPERGVHFVTTAPTLKLLNHHPLLEEFLQEGLEKGDEELRP |
| | | ALPFQPDPPAPFTPSPLPRLANQDSRPVFTSPTPAMAAVPTQPQS |
| | | KEGPWSPESESPMLRITAPLPPGPSMAVPTLGPGEIASTTPPSRA |
| | | WTPTQEGPGDMGRPWVAEVVSQGAGIGIQGTITSSTASGDDEETT |
| | | TTTTIITTTITTVQTPGPCSWNFSGPEGSLDSPTDLSSPTDVGLD |
| | | CFFYISVYPGYGVEIKVQNISLREGETVTVEGLGGPDPLPLANQS |
| | | FLLRGQVIRSPTHQAALRFQSLPPPAGPGTFHFHYQAYLLSCHFP |
| | | RRPAYGDVTVTSLHPGGSARFHCATGYQLKGARHLTCLNATQPFW |
| | | DSKEPVCIAACGGVIRNATTGRIVSPGFPGNYSNNLTCHWLLEAP |
| | | EGQRLHLHFEKVSLAEDDDRLIIRNGDNVEAPPVYDSYEVEYLPI |
| | | EGLLSSGKHFFVELSTDSSGAAAGMALRYEAFQQGHCYEPFVKYG |
| | | NFSSSTPTYPVGTTVEFSCDPGYTLEQGSIIIECVDPHDPQWNET |
| | | EPACRAVCSGEITDSAGVVLSPNWPEPYGRGQDCIWGVHVEEDKR |
| | | IMLDIRVLRIGPGDVLTFYDGDDLTARVLGQYSGPRSHFKLFTSM |
| | | ADVTIQFQSDPGTSVLGYQQGFVIHFFEVPRNDTCPELPEIPNGW |
| | | KSPSQPELVHGTVVTYQCYPGYQVVGSSVLMCQWDLTWSEDLPSC |
| | | QRVTSCHDPGDVEHSRRLISSPKFPVGATVQYICDQGFVLMGSSI |
| | | LTCHDRQAGSPKWSDRAPKCLLEQLKPCHGLSAPENGARSPEKQL |
| | | HPAGATIHFSCAPGYVLKGQASIKCVPGHPSHWSDPPPICRAASL |
| | | DGFYNSRSLDVAKAPAASSTLDAAHIAAAIFLPLVAMVLLVGGVY |
| | | FYFSRLQGKSSLQLPRPRPRPYNRITIESAFDNPTYETGSLSFAG |
| | | DERI |
| CD56 | 232 | MLQTKDLIWTLFFLGTAVSLQVDIVPSQGEISVGESKFFLCQVAG |
| | | DAKDKDISWFSPNGEKLTPNQQRISVVWNDDSSSTLTIYNANIDD |
| | | AGIYKCVVTGEDGSESEATVNVKIFQKLMFKNAPTPQEFREGEDA |
| | | VIVCDVVSSLPPTIIWKHKGRDVILKKDVRFIVLSNNYLQIRGIK |
| | | KTDEGTYRCEGRILARGEINFKDIQVIVNVPPTIQARQNIVNATA |
| | | NLGQSVTLVCDAEGFPEPTMSWTKDGEQIEQEEDDEKYIFSDDSS |
| | | QLTIKKVDKNDEAEYICIAENKAGEQDATIHLKVFAKPKITYVEN |
| | | QTAMELEEQVTLTCEASGDPIPSITWRISTRNISSEEKASWTRPE |
| | | KQETLDGHMVVRSHARVSSLTLKSIQYTDAGEYICTASNTIGQDS |
| | | QSMYLEVQYAPKLQGPVAVYTWEGNQVNITCEVFAYPSATISWER |
| | | DGQLLPSSNYSNIKIYNTPSASYLEVTPDSENDFGNYNCTAVNRI |
| | | GQESLEFILVQADTPSSPSIDQVEPYSSTAQVQFDEPEATGGVPI |
| | | LKYKAEWRAVGEEVWHSKWYDAKEASMEGIVTIVGLKPETTYAVR |
| | | LAALNGKGLGEISAASEFKTQPVQGEPSAPKLEGQMGEDGNSIKV |
| | | NLIKQDDGGSPIRHYLVRYRALSSEWKPEIRLPSGSDHVMLKSLD |
| | | WNAEYEVYVVAENQQGKSKAAHFVFRTSAQPTAIPANGSPTSGLS |
| | | TGAIVGILIVIFVLLLVVVDITCYFLNKCGLFMCIAVNLCGKAGP |
| | | GAKGKDMEEGKAAFSKDESKEPIVEVRTEEERTPNHDGGKHTEPN |
| | | ETTPLTEPEKGPVEAKPECQETETKPAPAEVKTVPNDATQTKENE |
| | | SKA |
| DLL3 | 233 | MVSPRMSGLLSQTVILALIFLPQTRPAGVFELQIHSFGPGPGPGA |
| | | PRSPCSARLPCRLFFRVCLKPGLSEEAAESPCALGAALSARGPVY |
| | | TEQPGAPAPDLPLPDGLLQVPFRDAWPGTFSFIIETWREELGDQI |
| | | GGPAWSLLARVAGRRRLAAGGPWARDIQRAGAWELRFSYRARCEP |
| | | PAVGTACTRLCRPRSAPSRCGPGLRPCAPLEDECEAPLVCRAGCS |
| | | PEHGFCEQPGECRCLEGWTGPLCTVPVSTSSCLSPRGPSSATTGC |
| | | LVPGPGPCDGNPCANGGSCSETPRSFECTCPRGFYGLRCEVSGVT |
| | | CADGPCFNGGLCVGGADPDSAYICHCPPGFQGSNCEKRVDRCSLQ |
| | | PCRNGGLCLDLGHALRCRCRAGFAGPRCEHDLDDCAGRACANGGT |
| | | CVEGGGAHRCSCALGFGGRDCRERADPCAARPCAHGGRCYAHFSG |
| | | LVCACAPGYMGARCEFPVHPDGASALPAAPPGLRPGDPQRYLLPP |
| | | ALGLLVAAGVAGAALLLVHVRRRGHSQDAGSRLLAGTPEPSVHAL |
| | | PDALNNLRTQEGSGDGPSSSVDWNRPEDVDPQGIYVISAPSIYAR |
| | | EVATPLFPPLHTGRAGQRQHLLFPYPSSILSVK |
| DLK1 | 234 | MTATEALLRVLLLLLAFGHSTYGAECFPACNPQNGFCEDDNVCRC |
| | | QPGWQGPLCDQCVTSPGCLHGLCGEPGQCICTDGWDGELCDRDVR |
| | | ACSSAPCANNRTCVSLDDGLYECSCAPGYSGKDCQKKDGPCVING |
| | | SPCQHGGTCVDDEGRASHASCLCPPGFSGNFCEIVANSCTPNPCE |
| | | NDGVCTDIGGDFRCRCPAGFIDKTCSRPVTNCASSPCQNGGTCLQ |
| | | HTQVSYECLCKPEFTGLTCVKKRALSPQQVTRLPSGYGLAYRLTP |
| | | GVHELPVQQPEHRILKVSMKELNKKTPLLTEGQAICFTILGVLTS |
| | | LVVLGTVGIVFLNKCETWVSNLRYNHMLRKKKNLLLQYNSGEDLA |
| | | VNIIFPEKIDMTTFSKEAGDEEI |
| B7H3 | 79 | MLRRRGSPGMGVHVGAALGALWFCLTGALEVQVPEDPVVALVGTD |
| | | ATLCCSFSPEPGFSLAQLNLIWQLTDTKQLVHSFAEGQDQGSAYA |
| | | NRTALFPDLLAQGNASLRLQRVRVADEGSFTCFVSIRDFGSAAVS |
| | | LQVAAPYSKPSMTLEPNKDLRPGDTVTITCSSYQGYPEAEVFWQD |
| | | GQGVPLTGNVTTSQMANEQGLFDVHSILRVVLGANGTYSCLVRNP |
| | | VLQQDAHSSVTITPQRSPTGAVEVQVPEDPVVALVGTDATLRCSF |
| | | SPEPGFSLAQLNLIWQLTDTKQLVHSFTEGRDQGSAYANRTALFP |
| | | DLLAQGNASLRLQRVRVADEGSFTCFVSIRDFGSAAVSLQVAAPY |
| | | SKPSMTLEPNKDLRPGDTVTITCSSYRGYPEAEVFWQDGQGVPLT |
| | | GNVTTSQMANEQGLFDVHSVLRVVLGANGTYSCLVRNPVLQQDAH |
| | | GSVTITGQPMTFPPEALWVTVGLSVCLIALLVALAFVCWRKIKQS |
| | | CEEENAGAEDQDGEGEGSKTALQPLKHSDSKEDDGQEIA |
| EphA2 | 337 | MELQAARACFALLWGCALAAAAAAQGKEVVLLDFAAAGGELGWLT |
| | | HPYGKGWDLMQNIMNDMPIYMYSVCNVMSGDQDNWLRTNWVYRGE |
| | | AERIFIELKFTVRDCNSFPGGASSCKETFNLYYAESDLDYGTNFQ |
| | | KRLFTKIDTIAPDEITVSSDFEARHVKLNVEERSVGPLTRKGFYL |
| | | AFQDIGACVALLSVRVYYKKCPELLQGLAHFPETIAGSDAPSLAT |
| | | VAGTCVDHAVVPPGGEEPRMHCAVDGEWLVPIGQCLCQAGYEKVE |
| | | DACQACSPGFFKFEASESPCLECPEHTLPSPEGATSCECEEGFFR |
| | | APQDPASMPCTRPPSAPHYLTAVGMGAKVELRWTPPQDSGGREDI |
| | | VYSVTCEQCWPESGECGPCEASVRYSEPPHGLTRTSVTVSDLEPH |
| | | MNYTFTVEARNGVSGLVTSRSFRTASVSINQTEPPKVRLEGRSTT |
| | | SLSVSWSIPPPQQSRVWKYEVTYRKKGDSNSYNVRRTEGFSVTLD |
| | | DLAPDTTYLVQVQALTQEGQGAGSKVHEFQTLSPEGSGNLAVIGG |
| | | VAVGVVLLLVLAGVGFFIHRRRKNQRARQSPEDVYFSKSEQLKPL |
| | | KTYVDPHTYEDPNQAVLKFTTEIHPSCVTRQKVIGAGEFGEVYKG |
| | | MLKTSSGKKEVPVAIKTLKAGYTEKQRVDFLGEAGIMGQFSHHNI |
| | | IRLEGVISKYKPMMIITEYMENGALDKFLREKDGEFSVLQLVGML |
| | | RGIAAGMKYLANMNYVHRDLAARNILVNSNLVCKVSDFGLSRVLE |
| | | DDPEATYTTSGGKIPIRWTAPEAISYRKFTSASDVWSFGIVMWEV |
| | | MTYGERPYWELSNHEVMKAINDGFRLPTPMDCPSAIYQLMMQCWQ |
| | | QERARRPKFADIVSILDKLIRAPDSLKTLADFDPRVSIRLPSTSG |
| | | SEGVPFRTVSEWLESIKMQQYTEHFMAAGYTAIEKVVQMINDDIK |
| | | RIGVRLPGHQKRIAYSLLGLKDQVNTVGIPI |

TABLE D7
Exemplary Antibody Sequences

| Ab | SEQ ID NO | IgG chain | Amino acid/Nucleotide sequence |
|---|---|---|---|
| CD74 | 118 | Heavy | QVQLQQSGSELKKPGASVKVSCKASGYTFTNYGVNWIK |
| milatuzumab | | Chain (Wild | QAPGQGLQWMGWINPNTGEPTFDDDFKGRFAFSLDTSV |
| Cysmab HC | | Type Fc) | STAYLQISSLKADDTAVYFCSRSRGKNEAWFAYWGQGT |
| (VHmil x | | | LVTVSSASTKGPSVFPLAPSSKSTSGGTAALGCLVKDY |
| VK1aNQ) | | | FPCPVTVSWNSGALTSGVHTFPAVLQSSGLYSLSSVVT |
| | | | VPSSSLGTQTYICNVNHKPSNTKVDKRVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPCDI |
| | | | AVEWESNGQPENNYKTTPPVLDSDGSFFLYSKLTVDKS |
| | | | RWQQGNVFSCSVMHEALHNHYTQKSLSLSPGK |
| | 235 | DNA Heavy | CAGGTTCAGCTCCAGCAGTCTGGCAGCGAGCTGAAAAA |
| | | Chain (Wild | ACCTGGCGCCTCCGTGAAGGTGTCCTGCAAGGCTTCTG |
| | | Type Fc) | GCTACACCTTTACCAACTACGGCGTGAACTGGATCAAG |
| | | | CAGGCCCCTGGACAAGGCCTCCAATGGATGGGCTGGAT |
| | | | CAACCCCAATACCGGCGAGCCCACCTTCGACGACGATT |
| | | | TCAAGGGCAGATTCGCCTTCAGCCTGGACACCTCTGTG |
| | | | TCCACAGCCTACCTCCAGATCAGCAGCCTGAAGGCCGA |
| | | | TGATACCGCCGTGTACTTCTGCTCCAGAAGCCGGGGAA |
| | | | AGAACGAGGCTTGGTTTGCCTATTGGGGCCAGGGCACA |
| | | | CTGGTCACCGTTAGCTCTGCTAGCACCAAGGGCCCAAG |
| | | | TGTGTTTCCCCTGGCCCCCAGCAGCAAGTCTACTTCCG |
| | | | GCGGAACTGCTGCCCTGGGTTGCCTGGTGAAGGACTAC |
| | | | TTCCCCTGTCCCGTGACAGTGTCCTGGAACTCTGGGGC |
| | | | TCTGACTTCCGGCGTGCACACCTTCCCCGCCGTGCTGC |
| | | | AGAGCAGCGGCCTGTACAGCCTGAGCAGCGTGGTGACA |
| | | | GTGCCCTCCAGCTCTCTGGGAACCCAGACCTATATCTG |
| | | | CAACGTGAACCACAAGCCCAGCAACACCAAGGTGGACA |
| | | | AGAGAGTGGAGCCCAAGAGCTGCGACAAGACCCACACC |
| | | | TGCCCCCCCTGCCCAGCTCCAGAACTGCTGGGAGGGCC |
| | | | TTCCGTGTTCCTGTTCCCCCCCAAGCCCAAGGACACCC |
| | | | TGATGATCAGCAGGACCCCCGAGGTGACCTGCGTGGTG |
| | | | GTGGACGTGTCCCACGAGGACCCAGAGGTGAAGTTCAA |
| | | | CTGGTACGTGGACGGCGTGGAGGTGCACAACGCCAAGA |
| | | | CCAAGCCCAGAGAGGAGCAGTACAACAGCACCTACAGG |
| | | | GTGGTGTCCGTGCTGACCGTGCTGCACCAGGACTGGCT |
| | | | GAACGGCAAAGAATACAAGTGCAAAGTCTCCAACAAGG |
| | | | CCCTGCCAGCCCCAATCGAAAAGACAATCAGCAAGGCC |
| | | | AAGGGCCAGCCACGGGAGCCCCAGGTGTACACCCTGCC |
| | | | CCCCAGCCGGGAGGAGATGACCAAGAACCAGGTGTCCC |
| | | | TGACCTGTCTGGTGAAGGGCTTCTACCCCTGTGATATC |
| | | | GCCGTGGAGTGGGAGAGCAACGGCCAGCCCGAGAACAA |
| | | | CTACAAGACCACCCCCCCAGTGCTGGACAGCGACGGCA |
| | | | GCTTCTTCCTGTACAGCAAGCTGACCGTGGACAAGTCC |
| | | | AGGTGGCAGCAGGGCAACGTGTTCAGCTGCAGCGTGAT |
| | | | GCACGAGGCCCTGCACAACCACTACACCCAGAAGTCCC |
| | | | TGAGCCTGAGCCCCGGCAAG |
| CD74 | 236 | Heavy | QVQLQQSGSELKKPGASVKVSCKASGYTFTNYGVNWIK |
| Milatuzumab | | Chain (Fc | QAPGQGLQWMGWINPNTGEPTFDDDFKGRFAFSLDTSV |
| DANAPA | | Silenced | STAYLQISSLKADDTAVYFCSRSRGKNEAWFAYWGQGT |
| Cysmab | | DANAPA) | LVTVSSASTKGPSVFPLAPSSKSTSGGTAALGCLVKDY |
| | | | FPCPVTVSWNSGALTSGVHTFPAVLQSSGLYSLSSVVT |
| | | | VPSSSLGTQTYICNVNHKPSNTKVDKRVEPKSCDKTHT |
| | | | CPPCPAPELLGGPSVFLFPPKPKDTLMISRTPEVTCVV |
| | | | VAVSHEDPEVKFNWYVDGVEVHNAKTKPREEQYASTYR |
| | | | VVSVLTVLHQDWLNGKEYKCKVSNKALAAPIEKTISKA |
| | | | KGQPREPQVYTLPPSREEMTKNQVSLTCLVKGFYPCDI |
| | | | AVEWESNGQPENNYKTTPPVLDSDGSFFLYSKLTVDKS |
| | | | RWQQGNVFSCSVMHEALHNHYTQKSLSLSPGK |
| CD74 | 237 | Light Chain | DIQLTQSPLSLPVTLGQPASISCRSSQSLVHRNGNTYL |
| Milatuzumab | | | HWFQQRPGQSPRLLIYTVSNRFSGVPDRFSGSGSGTDF |
| LC | | | TLKISRVEAEDVGVYFCSQSSHVPPTFGAGTRLEIKRT |
| | | | VAAPSVFIFPPSDEQLKSGTASVVCLLNNFYPREAKVQ |
| | | | WKVDNALQSGNSQESVTEQDSKDSTYSLSSTLTLSKAD |
| | | | YEKHKVYACEVTHQGLSSPVTKSFNRGEC |
| | 238 | DNA Light | GACATTCAGCTGACACAGAGCCCTCTGAGCCTGCCTGT |
| | | Chain | TACACTGGGACAGCCTGCCAGCATCAGCTGTAGAAGCA |
| | | | GCCAGAGCCTGGTGCACAGAAACGGCAACACCTACCTG |
| | | | CACTGGTTCCAGCAGAGGCCTGGCCAGTCTCCTAGACT |
| | | | GCTGATCTACACCGTGTCCAACAGATTCAGCGGCGTGC |
| | | | CCGATAGATTTTCCGGCAGCGGCTCTGGCACCGACTTC |
| | | | ACCCTGAAGATTAGCAGAGTGGAAGCCGAGGACGTGGG |
| | | | CGTGTACTTCTGTAGCCAGTCTAGCCACGTGCCACCTA |
| | | | CCTTTGGCGCCGGAACCAGACTGGAAATCAAGCGTACG |
| | | | GTGGCCGCTCCCAGCGTGTTCATCTTCCCCCCCAGCGA |
| | | | CGAGCAGCTGAAGAGTGGCACCGCCAGCGTGGTGTGCC |
| | | | TGCTGAACAACTTCTACCCCCGGGAGGCCAAGGTGCAG |
| | | | TGGAAGGTGGACAACGCCCTGCAGAGCGGCAACAGCCA |
| | | | GGAGAGCGTCACCGAGCAGGACAGCAAGGACTCCACCT |
| | | | ACAGCCTGAGCAGCACCCTGACCCTGAGCAAGGCCGAC |
| | | | TACGAGAAGCATAAGGTGTACGCCTGCGAGGTGACCCA |
| | | | CCAGGGCCTGTCCAGCCCCGTGACCAAGAGCTTCAACA |
| | | | GGGGCGAGTGC |
| CD74 LC | 239 | Light Chain | DIVMTQTPLSLPVTPGEPASISCRSSQSLVHRNQNTYL |
| Cysmab WT | | | HWYLQKPGQSPQLLIYTVSNRFSGVPDRFSGSGSGTDF |
| (VHmil x | | | TLKSRVEAEDVGVYFCSQSSHVPPTFGQGTKLEIKRTV |
| VK1aNQ) | | | AAPSVFIFPPSDEQLKSGTASVVCLLNNFYPREAKVQW |
| | | | KVDNALSGNSQESVTEQDSKDSTYSLSSTLTLSKADYE |
| | | | KHKVYACEVTHQGLSSPVTKSFNRGEC |
| CD48 NY920 | 240 | Heavy | EVQLLESGGGLVQPGGSLRLSCAASGFTFSSFAMSWVR |
| Cysmab WT | | Chain (Wild | QAPGKGLEWVSAISGFGGSTYYADSVKGRFTISRDNSK |
| HC | | Type Fc) | NTLYLQMNSLRAEDTAVYYCARQFWEDQPFYFDYWGQG |
| | | | TLVTVSSASTKGPSVFPLAPSSKSTSGGTAALGCLVKD |
| | | | YFPCPVTVSWNSGALTSGVHTFPAVLQSSGLYSLSSVV |
| | | | TVPSSSLGTQTYICNVNHKPSNTKVDKRVEPKSCDKTH |
| | | | TCPPCPAPELLGGPSVFLFPPKPKDTLMISRTPEVTCV |
| | | | VVDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSREEMTKNQVSLTCLVKGFYPCD |
| | | | IAVEWESNGQPENNYKTTPPVLDSDGSFFLYSKLTVDK |
| | | | SRWQQGNVFSCSVMHEALHNHYTQKSLSLSPGK |
| | 241 | DNA Heavy | GAGGTGCAGCTGCTGGAGAGCGGCGGCGGCCTGGTGCA |
| | | Chain | GCCCGGCGGCAGCCTGCGGCTGAGCTGCGCCGCCAGCG |
| | | | GCTTCACCTTCAGCAGCTTCGCCATGAGCTGGGTGCGG |
| | | | CAGGCCCCCGGCAAGGGCCTGGAGTGGGTGAGCGCCAT |
| | | | CAGCGGCTTCGGCGGCAGCACCTACTACGCCGACAGCG |
| | | | TGAAGGGCCGGTTCACCATCAGCCGGGACAACAGCAAG |
| | | | AACACCCTGTACCTGCAGATGAACAGCCTGCGGGCCGA |
| | | | GGACACCGCCGTGTACTACTGCGCCCGGCAGTTCTGGG |
| | | | AGGACCAGCCCTTCTACTTCGACTACTGGGGCCAGGGC |
| | | | ACCCTGGTGACCGTGAGCAGCGCCAGCACCAAGGGCCC |
| | | | CAGCGTGTTCCCCCTGGCCCCCAGCAGCAAGAGCACCA |
| | | | GCGGCGGCACCGCCGCCCTGGGCTGCCTGGTGAAGGAC |
| | | | TACTTCCCCTGCCCCGTGACCGTGAGCTGGAACAGCGG |
| | | | CGCCCTGACCAGCGGCGTGCACACCTTCCCCGCCGTGC |
| | | | TGCAGAGCAGCGGCCTGTACAGCCTGAGCAGCGTGGTG |
| | | | ACCGTGCCCAGCAGCAGCCTGGGCACCCAGACCTACAT |
| | | | CTGCAACGTGAACCACAAGCCCAGCAACACCAAGGTGG |
| | | | ACAAGCGGGTGGAGCCCAAGAGCTGCGACAAGACCCAC |
| | | | ACCTGCCCCCCCTGCCCCGCCCCCGAGCTGCTGGGCGG |
| | | | CCCCAGCGTGTTCCTGTTCCCCCCCAAGCCCAAGGACA |
| | | | CCCTGATGATCAGCCGGACCCCCGAGGTGACCTGCGTG |
| | | | GTGGTGGACGTGAGCCACGAGGACCCCGAGGTGAAGTT |
| | | | CAACTGGTACGTGGACGGCGTGGAGGTGCACAACGCCA |
| | | | AGACCAAGCCCCGGGAGGAGCAGTACAACAGCACCTAC |
| | | | CGGGTGGTGAGCGTGCTGACCGTGCTGCACCAGGACTG |
| | | | GCTGAACGGCAAGGAGTACAAGTGCAAGGTGAGCAACA |
| | | | AGGCCCTGCCCGCCCCCATCGAGAAGACCATCAGCAAG |
| | | | GCCAAGGGCCAGCCCCGGGAGCCCCAGGTGTACACCCT |
| | | | GCCCCCCAGCCGGGAGGAGATGACCAAGAACCAGGTGA |
| | | | GCCTGACCTGCCTGGTGAAGGGCTTCTACCCCTGCGAC |
| | | | ATCGCCGTGGAGTGGGAGAGCAACGGCCAGCCCGAGAA |
| | | | CAACTACAAGACCACCCCCCCCGTGCTGGACAGCGACG |
| | | | GCAGCTTCTTCCTGTACAGCAAGCTGACCGTGGACAAG |
| | | | AGCCGGTGGCAGCAGGGCAACGTGTTCAGCTGCAGCGT |
| | | | GATGCACGAGGCCCTGCACAACCACTACACCCAGAAGA |
| | | | GCCTGAGCCTGAGCCCCGGCAAG |
| CD48 NY920 | 242 | | EVQLLESGGGLVQPGGSLRLSCAASGFTFSSFAMSWVR |
| Cysmab DAPA | | | QAPGKGLEWVSAISGFGGSTYYADSVKGRFTISRDNSK |
| HC | | | NTLYLQMNSLRAEDTAVYYCARQFWEDQPFYFDYWGQG |
| | | | TLVTVSSASTKGPSVFPLAPSSKSTSGGTAALGCLVKD |
| | | | YFPCPVTVSWNSGALTSGVHTFPAVLQSSGLYSLSSVV |
| | | | TVPSSSLGTQTYICNVNHKPSNTKVDKRVEPKSCDKTH |
| | | | TCPPCPAPELLGGPSVFLFPPKPKDTLMISRTPEVTCV |
| | | | VVAVSHEDPEVKFNWYVDGVEVHNAKTKPREEQYNSTY |
| | | | RVVSVLTVLHQDWLNGKEYKCKVSNKALAAPIEKTISK |
| | | | AKGQPREPQVYTLPPSREEMTKNQVSLTCLVKGFYPCD |
| | | | IAVEWESNGQPENNYKTTPPVLDSDGSFFLYSKLTVDK |
| | | | SRWQQGNVFSCSVMHEALHNHYTQKSLSLSPGK |
| CD48 NY920 | 243 | Light Chain | DIQMTQSPSSLSASVGDRVTITCRASQSISSYLNWYQQ |
| LC | | | KPGKAPKLLIYAASSLQSGVPSRFSGSGSGTDFTLTIS |
| | | | SLQPEDFATYYCQQSYSTPLTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 244 | DNA Light | GACATCCAGATGACCCAGAGCCCCAGCAGCCTGAGCGC |
| | | Chain | CAGCGTGGGCGACCGGGTGACCATCACCTGCCGGGCCA |
| | | | GCCAGAGCATCAGCAGCTACCTGAACTGGTACCAGCAG |
| | | | AAGCCCGGCAAGGCCCCCAAGCTGCTGATCTACGCCGC |
| | | | CAGCAGCCTGCAGAGCGGCGTGCCCAGCCGGTTCAGCG |
| | | | GCAGCGGCAGCGGCACCGACTTCACCCTGACCATCAGC |
| | | | AGCCTGCAGCCCGAGGACTTCGCCACCTACTACTGCCA |
| | | | GCAGAGCTACAGCACCCCCCTGACCTTCGGCCAGGGCA |
| | | | CCAAGGTGGAGATCAAGCGGACCGTGGCCGCCCCCAGC |
| | | | GTGTTCATCTTCCCCCCCAGCGACGAGCAGCTGAAGAG |
| | | | CGGCACCGCCAGCGTGGTGTGCCTGCTGAACAACTTCT |
| | | | ACCCCCGGGAGGCCAAGGTGCAGTGGAAGGTGGACAAC |
| | | | GCCCTGCAGAGCGGCAACAGCCAGGAGAGCGTGACCGA |
| | | | GCAGGACAGCAAGGACAGCACCTACAGCCTGAGCAGCA |
| | | | CCCTGACCCTGAGCAAGGCCGACTACGAGAAGCACAAG |
| | | | GTGTACGCCTGCGAGGTGACCCACCAGGGCCTGAGCAG |
| | | | CCCCGTGACCAAGAGCTTCAACCGGGGCGAGTGC |
| Her2 Cysmab | 245 | Heavy | EVQLVESGGGLVQPGGSLRLSCAASGFNIKDTYIHWVR |
| WT HC | | Chain (Wild | QAPGKGLEWVARIYPTNGYTRYADSVKGRFTISADTSK |
| | | Type Fc) | NTAYLQMNSLRAEDTAVYYCSRWGGDGFYAMDYWGQGT |
| | | | LVTVSSASTKGPSVFPLAPSSKSTSGGTAALGCLVKDY |
| | | | FPCPVTVSWNSGALTSGVHTFPAVLQSSGLYSLSSVVT |
| | | | VPSSSLGTQTYICNVNHKPSNTKVDKRVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPCDI |
| | | | AVEWESNGQPENNYKTTPPVLDSDGSFFLYSKLTVDKS |
| | | | RWQQGNVFSCSVMHEALHNHYTQKSLSLSPGK |
| | 246 | DNA Heavy | GAGGTGCAGCTGGTGGAGAGCGGCGGCGGCCTGGTGCA |
| | | Chain | GCCCGGCGGCAGCCTGCGGCTGAGCTGCGCCGCCAGCG |
| | | | GCTTCAACATCAAGGACACCTACATCCACTGGGTGCGG |
| | | | CAGGCCCCCGGCAAGGGCCTGGAGTGGGTGGCCCGGAT |
| | | | CTACCCCACCAACGGCTACACCCGGTACGCCGACAGCG |
| | | | TGAAGGGCCGGTTCACCATCAGCGCCGACACCAGCAAG |
| | | | AACACCGCCTACCTGCAGATGAACAGCCTGCGGGCCGA |
| | | | GGACACCGCCGTGTACTACTGCAGCCGGTGGGGCGGCG |
| | | | ACGGCTTCTACGCCATGGACTACTGGGGCCAGGGCACC |
| | | | CTGGTGACCGTGAGCAGCGCCAGCACCAAGGGCCCCAG |
| | | | CGTGTTCCCCCTGGCCCCCAGCAGCAAGAGCACCAGCG |
| | | | GCGGCACCGCCGCCCTGGGCTGCCTGGTGAAGGACTAC |
| | | | TTCCCCTGCCCCGTGACCGTGAGCTGGAACAGCGGCGC |
| | | | CCTGACCAGCGGCGTGCACACCTTCCCCGCCGTGCTGC |
| | | | AGAGCAGCGGCCTGTACAGCCTGAGCAGCGTGGTGACC |
| | | | GTGCCCAGCAGCAGCCTGGGCACCCAGACCTACATCTG |
| | | | CAACGTGAACCACAAGCCCAGCAACACCAAGGTGGACA |
| | | | AGCGGGTGGAGCCCAAGAGCTGCGACAAGACCCACACC |
| | | | TGCCCCCCCTGCCCCGCCCCCGAGCTGCTGGGCGGCCC |
| | | | CAGCGTGTTCCTGTTCCCCCCCAAGCCCAAGGACACCC |
| | | | TGATGATCAGCCGGACCCCCGAGGTGACCTGCGTGGTG |
| | | | GTGGACGTGAGCCACGAGGACCCCGAGGTGAAGTTCAA |
| | | | CTGGTACGTGGACGGCGTGGAGGTGCACAACGCCAAGA |
| | | | CCAAGCCCCGGGAGGAGCAGTACAACAGCACCTACCGG |
| | | | GTGGTGAGCGTGCTGACCGTGCTGCACCAGGACTGGCT |
| | | | GAACGGCAAGGAGTACAAGTGCAAGGTGAGCAACAAGG |
| | | | CCCTGCCCGCCCCCATCGAGAAGACCATCAGCAAGGCC |
| | | | AAGGGCCAGCCCCGGGAGCCCCAGGTGTACACCCTGCC |
| | | | CCCCAGCCGGGAGGAGATGACCAAGAACCAGGTGAGCC |
| | | | TGACCTGCCTGGTGAAGGGCTTCTACCCCTGCGACATC |
| | | | GCCGTGGAGTGGGAGAGCAACGGCCAGCCCGAGAACAA |
| | | | CTACAAGACCACCCCCCCCGTGCTGGACAGCGACGGCA |
| | | | GCTTCTTCCTGTACAGCAAGCTGACCGTGGACAAGAGC |
| | | | CGGTGGCAGCAGGGCAACGTGTTCAGCTGCAGCGTGAT |
| | | | GCACGAGGCCCTGCACAACCACTACACCCAGAAGAGCC |
| | | | TGAGCCTGAGCCCCGGCAAG |
| | 66 | Light Chain | DIQMTQSPSSLSASVGDRVTITCRASQDVNTAVAWYQQ |
| | | | KPGKAPKLLIYSASFLYSGVPSRFSGSRSGTDFTLTIS |
| | | | SLQPEDFATYYCQQHYTTPPTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 247 | DNA Light | GACATCCAGATGACCCAGAGCCCCAGCAGCCTGAGCGC |
| | | Chain | CAGCGTGGGCGACCGGGTGACCATCACCTGCCGGGCCA |
| | | | GCCAGGACGTGAACACCGCCGTGGCCTGGTACCAGCAG |
| | | | AAGCCCGGCAAGGCCCCCAAGCTGCTGATCTACAGCGC |
| | | | CAGCTTCCTGTACAGCGGCGTGCCCAGCCGGTTCAGCG |
| | | | GCAGCCGGAGCGGCACCGACTTCACCCTGACCATCAGC |
| | | | AGCCTGCAGCCCGAGGACTTCGCCACCTACTACTGCCA |
| | | | GCAGCACTACACCACCCCCCCCACCTTCGGCCAGGGCA |
| | | | CCAAGGTGGAGATCAAGCGGACCGTGGCCGCCCCCAGC |
| | | | GTGTTCATCTTCCCCCCCAGCGACGAGCAGCTGAAGAG |
| | | | CGGCACCGCCAGCGTGGTGTGCCTGCTGAACAACTTCT |
| | | | ACCCCCGGGAGGCCAAGGTGCAGTGGAAGGTGGACAAC |
| | | | GCCCTGCAGAGCGGCAACAGCCAGGAGAGCGTGACCGA |
| | | | GCAGGACAGCAAGGACAGCACCTACAGCCTGAGCAGCA |
| | | | CCCTGACCCTGAGCAAGGCCGACTACGAGAAGCACAAG |
| | | | GTGTACGCCTGCGAGGTGACCCACCAGGGCCTGAGCAG |
| | | | CCCCGTGACCAAGAGCTTCAACCGGGGCGAGTGC |
| PCAD | 248 | Heavy | EVQLVQSGAEVKKPGESLKISCKVSGYTFTDHTIHWMR |
| CQY679 | | Chain (Wild | QMPGKGLEWMGYIYPRSGSINYNEKFKGQVTISADKSS |
| Cysmab WT | | Type Fc) | STAYLQWSSLKASDTAMYYCARRNLFLPMEYWGQGTLV |
| HC | | | TVSSASTKGPSVFPLAPSSKSTSGGTAALGCLVKDYFP |
| | | | EPVTVSWNSGALTSGVHTFPAVLQSSGLYSLSSVVTVP |
| | | | SSSLGTQTYICNVNHKPSNTKVDKRVEPKSCDKTHTCP |
| | | | PCPAPELLGGPSVFLFPPKPKDTLMISRTPEVTCVVVD |
| | | | VSHEDPEVKFNWYVDGVEVHNAKTKPREEQYNSTYRVV |
| | | | SVLTVLHQDWLNGKEYKCKVSNKALPAPIEKTISKAKG |
| | | | QPREPQVYTLPPSREEMTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| | 249 | DNA Heavy | GAGGTGCAGCTGGTGCAGAGCGGCGCCGAGGTGAAGAA |
| | | Chain | GCCCGGCGAGAGCCTGAAGATCAGCTGCAAGGTGAGCG |
| | | | GCTACACCTTCACCGACCACACCATCCACTGGATGCGG |
| | | | CAGATGCCCGGCAAGGGCCTGGAGTGGATGGGCTACAT |
| | | | CTACCCCCGGAGCGGCAGCATCAACTACAACGAGAAGT |
| | | | TCAAGGGCCAGGTGACCATCAGCGCCGACAAGAGCAGC |
| | | | AGCACCGCCTACCTGCAGTGGAGCAGCCTGAAGGCCAG |
| | | | CGACACCGCCATGTACTACTGCGCCCGGCGGAACCTGT |
| | | | TCCTGCCCATGGAGTACTGGGGCCAGGGCACCCTGGTG |
| | | | ACCGTGAGCAGCGCCAGCACCAAGGGCCCCAGCGTGTT |
| | | | CCCCCTGGCCCCCAGCAGCAAGAGCACCAGCGGCGGCA |
| | | | CCGCCGCCCTGGGCTGCCTGGTGAAGGACTACTTCCCC |
| | | | GAGCCCGTGACCGTGAGCTGGAACAGCGGCGCCCTGAC |
| | | | CAGCGGCGTGCACACCTTCCCCGCCGTGCTGCAGAGCA |
| | | | GCGGCCTGTACAGCCTGAGCAGCGTGGTGACCGTGCCC |
| | | | AGCAGCAGCCTGGGCACCCAGACCTACATCTGCAACGT |
| | | | GAACCACAAGCCCAGCAACACCAAGGTGGACAAGCGGG |
| | | | TGGAGCCCAAGAGCTGCGACAAGACCCACACCTGCCCC |
| | | | CCCTGCCCCGCCCCCGAGCTGCTGGGCGGCCCCAGCGT |
| | | | GTTCCTGTTCCCCCCCAAGCCCAAGGACACCCTGATGA |
| | | | TCAGCCGGACCCCCGAGGTGACCTGCGTGGTGGTGGAC |
| | | | GTGAGCCACGAGGACCCCGAGGTGAAGTTCAACTGGTA |
| | | | CGTGGACGGCGTGGAGGTGCACAACGCCAAGACCAAGC |
| | | | CCCGGGAGGAGCAGTACAACAGCACCTACCGGGTGGTG |
| | | | AGCGTGCTGACCGTGCTGCACCAGGACTGGCTGAACGG |
| | | | CAAGGAGTACAAGTGCAAGGTGAGCAACAAGGCCCTGC |
| | | | CCGCCCCCATCGAGAAGACCATCAGCAAGGCCAAGGGC |
| | | | CAGCCCCGGGAGCCCCAGGTGTACACCCTGCCCCCCAG |
| | | | CCGGGAGGAGATGACCAAGAACCAGGTGAGCCTGACCT |
| | | | GCCTGGTGAAGGGCTTCTACCCCAGCGACATCGCCGTG |
| | | | GAGTGGGAGAGCAACGGCCAGCCCGAGAACAACTACAA |
| | | | GACCACCCCCCCCGTGCTGGACAGCGACGGCAGCTTCT |
| | | | TCCTGTACAGCAAGCTGACCGTGGACAAGAGCCGGTGG |
| | | | CAGCAGGGCAACGTGTTCAGCTGCAGCGTGATGCACGA |
| | | | GGCCCTGCACAACCACTACACCCAGAAGAGCCTGAGCC |
| | | | TGAGCCCCGGCAAG |
| | 250 | Light Chain | DIVMTQTPLSLPVTPGEPASISCRSSQSLLSSGDQKNY |
| | | | LTWYLQKPGQSPQLLIYWASTRESGVPDRFSGSGSGTD |
| | | | FTLKISRVEAEDVGVYYCQNDYRYPLTFGQGTKLEIKR |
| | | | TVAAPSVFIFPPSDEQLKSGTASVVCLLNNFYPREAKV |
| | | | QWKVDNALQSGNSQESVTEQDSKDSTYSLSSTLTLSKA |
| | | | DYEKHKVYACEVTHQGLSSPVTKSFNRGEC |
| | 251 | DNA Light | GACATCGTGATGACCCAGACCCCCCTGAGCCTGCCCGT |
| | | Chain | GACCCCCGGCGAGCCCGCCAGCATCAGCTGCCGGAGCA |
| | | | GCCAGAGCCTGCTGAGCAGCGGCGACCAGAAGAACTAC |
| | | | CTGACCTGGTACCTGCAGAAGCCCGGCCAGAGCCCCCA |
| | | | GCTGCTGATCTACTGGGCCAGCACCCGGGAGAGCGGCG |
| | | | TGCCCGACCGGTTCAGCGGCAGCGGCAGCGGCACCGAC |
| | | | TTCACCCTGAAGATCAGCCGGGTGGAGGCCGAGGACGT |
| | | | GGGCGTGTACTACTGCCAGAACGACTACCGGTACCCCC |
| | | | TGACCTTCGGCCAGGGCACCAAGCTGGAGATCAAGCGG |
| | | | ACCGTGGCCGCCCCCAGCGTGTTCATCTTCCCCCCCAG |
| | | | CGACGAGCAGCTGAAGAGCGGCACCGCCAGCGTGGTGT |
| | | | GCCTGCTGAACAACTTCTACCCCCGGGAGGCCAAGGTG |
| | | | CAGTGGAAGGTGGACAACGCCCTGCAGAGCGGCAACAG |
| | | | CCAGGAGAGCGTGACCGAGCAGGACAGCAAGGACAGCA |
| | | | CCTACAGCCTGAGCAGCACCCTGACCCTGAGCAAGGCC |
| | | | GACTACGAGAAGCACAAGGTGTACGCCTGCGAGGTGAC |
| | | | CCACCAGGGCCTGAGCAGCCCCGTGACCAAGAGCTTCA |
| | | | ACCGGGGCGAGTGC |
| EphA2 1C1 | 252 | Heavy | EVQLLESGGGLVQPGGSLRLSCAASGFTFSHYMMAWVR |
| Cysmab WT | | Chain (Wild | QAPGKGLEWVSRIGPSGGPTHYADSVKGRFTISRDNSK |
| HC | | Type Fc) | NTLYLQMNSLRAEDTAVYYCAGYDSGYDYVAVAGPAEY |
| | | | FQHWGQGTLVTVSSASTKGPSVFPLAPSSKSTSGGTAA |
| | | | LGCLVKDYFPCPVTVSWNSGALTSGVHTFPAVLQSSGL |
| | | | YSLSSVVTVPSSSLGTQTYICNVNHKPSNTKVDKRVEP |
| | | | KSCDKTHTCPPCPAPELLGGPSVFLFPPKPKDTLMISR |
| | | | TPEVTCVVVDVSHEDPEVKFNWYVDGVEVHNAKTKPRE |
| | | | EQYNSTYRVVSVLTVLHQDWLNGKEYKCKVSNKALPAP |
| | | | IEKTISKAKGQPREPQVYTLPPSREEMTKNQVSLTCLV |
| | | | KGFYPCDIAVEWESNGQPENNYKTTPPVLDSDGSFFLY |
| | | | SKLTVDKSRWQQGNVFSCSVMHEALHNHYTQKSLSLSP |
| | | | GK |
| | 253 | DNA Heavy | GAAGTTCAGCTGCTTGAATCTGGCGGCGGACTGGTTCA |
| | | Chain | ACCTGGCGGATCTCTGAGACTGAGCTGTGCCGCCAGCG |
| | | | GCTTCACCTTCAGCCACTATATGATGGCCTGGGTCCGA |
| | | | CAGGCCCCTGGCAAAGGACTTGAATGGGTGTCCAGAAT |
| | | | CGGCCCCTCTGGCGGCCCTACACACTACGCTGATTCTG |
| | | | TGAAGGGCAGATTCACCATCAGCCGGGACAACAGCAAG |
| | | | AACACCCTGTACCTGCAGATGAACAGCCTGAGAGCCGA |
| | | | GGACACCGCCGTGTATTACTGTGCCGGCTACGACAGCG |
| | | | GCTACGATTATGTGGCTGTGGCCGGACCTGCCGAGTAC |
| | | | TTTCAGCATTGGGGACAGGGCACCCTGGTCACCGTTAG |
| | | | TTCTGCTAGCACCAAGGGCCCAAGTGTGTTTCCCCTGG |
| | | | CCCCCAGCAGCAAGTCTACTTCCGGCGGAACTGCTGCC |
| | | | CTGGGTTGCCTGGTGAAGGACTACTTCCCCTGTCCCGT |
| | | | GACAGTGTCCTGGAACTCTGGGGCTCTGACTTCCGGCG |
| | | | TGCACACCTTCCCCGCCGTGCTGCAGAGCAGCGGCCTG |
| | | | TACAGCCTGAGCAGCGTGGTGACAGTGCCCTCCAGCTC |
| | | | TCTGGGAACCCAGACCTATATCTGCAACGTGAACCACA |
| | | | AGCCCAGCAACACCAAGGTGGACAAGAGAGTGGAGCCC |
| | | | AAGAGCTGCGACAAGACCCACACCTGCCCCCCCTGCCC |
| | | | AGCTCCAGAACTGCTGGGAGGGCCTTCCGTGTTCCTGT |
| | | | TCCCCCCCAAGCCCAAGGACACCCTGATGATCAGCAGG |
| | | | ACCCCCGAGGTGACCTGCGTGGTGGTGGACGTGTCCCA |
| | | | CGAGGACCCAGAGGTGAAGTTCAACTGGTACGTGGACG |
| | | | GCGTGGAGGTGCACAACGCCAAGACCAAGCCCAGAGAG |
| | | | GAGCAGTACAACAGCACCTACAGGGTGGTGTCCGTGCT |
| | | | GACCGTGCTGCACCAGGACTGGCTGAACGGCAAAGAAT |
| | | | ACAAGTGCAAAGTCTCCAACAAGGCCCTGCCAGCCCCA |
| | | | ATCGAAAAGACAATCAGCAAGGCCAAGGGCCAGCCACG |
| | | | GGAGCCCCAGGTGTACACCCTGCCCCCCAGCCGGGAGG |
| | | | AGATGACCAAGAACCAGGTGTCCCTGACCTGTCTGGTG |
| | | | AAGGGCTTCTACCCCTGTGATATCGCCGTGGAGTGGGA |
| | | | GAGCAACGGCCAGCCCGAGAACAACTACAAGACCACCC |
| | | | CCCCAGTGCTGGACAGCGACGGCAGCTTCTTCCTGTAC |
| | | | AGCAAGCTGACCGTGGACAAGTCCAGGTGGCAGCAGGG |
| | | | CAACGTGTTCAGCTGCAGCGTGATGCACGAGGCCCTGC |
| | | | ACAACCACTACACCCAGAAGTCCCTGAGCCTGAGCCCC |
| | | | GGCAAG |
| | 254 | Light Chain | DIQMTQSPSSLSASVGDRVTITCRASQSISTWLAWYQQ |
| | | | KPGKAPKLLIYKASNLHTGVPSRFSGSGSGTEFSLTIS |
| | | | GLQPDDFATYYCQQYNSYSRTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 255 | DNA Light | GACATTCAGATGACACAGAGCCCTAGCAGCCTGAGCGC |
| | | Chain | CTCTGTGGGAGACAGAGTGACCATCACCTGTAGAGCCA |
| | | | GCCAGAGCATCAGCACATGGCTGGCCTGGTATCAGCAG |
| | | | AAGCCTGGCAAGGCCCCTAAGCTGCTGATCTACAAGGC |
| | | | CAGCAATCTGCACACCGGCGTGCCCAGCAGATTTTCTG |
| | | | GCTCTGGAAGCGGCACCGAGTTCAGCCTGACAATCTCT |
| | | | GGCCTGCAGCCTGACGACTTCGCCACCTACTACTGCCA |
| | | | GCAGTACAACAGCTACAGCCGGACCTTTGGCCAGGGAA |
| | | | CAAAGGTGGAAATCAAGCGTACGGTGGCCGCTCCCAGC |
| | | | GTGTTCATCTTCCCCCCCAGCGACGAGCAGCTGAAGAG |
| | | | TGGCACCGCCAGCGTGGTGTGCCTGCTGAACAACTTCT |
| | | | ACCCCCGGGAGGCCAAGGTGCAGTGGAAGGTGGACAAC |
| | | | GCCCTGCAGAGCGGCAACAGCCAGGAGAGCGTCACCGA |
| | | | GCAGGACAGCAAGGACTCCACCTACAGCCTGAGCAGCA |
| | | | CCCTGACCCTGAGCAAGGCCGACTACGAGAAGCATAAG |
| | | | GTGTACGCCTGCGAGGTGACCCACCAGGGCCTGTCCAG |
| | | | CCCCGTGACCAAGAGCTTCAACAGGGGCGAGTGC |
| HER2 | 377 | Heavy | EVQLVQSGAEVKKPGATVKISCKVSGYTFTDYYIHWVQ |
| Disitamab | | Chain | QAPGKGLEWMGRVNPDHGDSYYNQKFKDKATITADKST |
| | | (E152C | DTAYMELSSLRSEDTAVYFCARNYLFDHWGQGTLVTVS |
| | | S375C) | SASTKGPSVFPLAPSSKSTSGGTAALGCLVKDYFPCPV |
| | | | TVSWNSGALTSGVHTFPAVLQSSGLYSLSSVVTVPSSS |
| | | | LGTQTYICNVNHKPSNTKVDKRVEPKSCDKTHTCPPCP |
| | | | APELLGGPSVFLFPPKPKDTLMISRTPEVTCVVVDVSH |
| | | | EDPEVKFNWYVDGVEVHNAKTKPREEQYNSTYRVVSVL |
| | | | TVLHQDWLNGKEYKCKVSNKALPAPIEKTISKAKGQPR |
| | | | EPQVYTLPPSREEMTKNQVSLTCLVKGFYPCDIAVEWE |
| | | | SNGQPENNYKTTPPVLDSDGSFFLYSKLTVDKSRWQQG |
| | | | NVFSCSVMHEALHNHYTQKSLSLSPGK |
| | 378 | DNA Heavy | GAAGTTCAGCTGGTTCAGTCTGGCGCCGAAGTGAAGAA |
| | | Chain | ACCTGGCGCCACCGTGAAGATCAGCTGCAAGGTGTCCG |
| | | (E152C | GCTACACCTTCACCGACTACTACATCCACTGGGTGCAG |
| | | S375C) | CAGGCCCCTGGCAAAGGACTTGAGTGGATGGGCAGAGT |
| | | | GAACCCCGATCACGGCGACAGCTACTACAACCAGAAGT |
| | | | TCAAGGACAAGGCCACCATCACCGCCGACAAGAGCACC |
| | | | GATACCGCCTACATGGAACTGAGCAGCCTGAGAAGCGA |
| | | | GGATACCGCCGTGTACTTCTGCGCCCGGAACTACCTGT |
| | | | TTGACCACTGGGGACAGGGCACCCTGGTCACAGTTAGT |
| | | | TCTGCTAGCACCAAGGGCCCAAGTGTGTTTCCCCTGGC |
| | | | CCCCAGCAGCAAGTCTACTTCCGGCGGAACTGCTGCCC |
| | | | TGGGTTGCCTGGTGAAGGACTACTTCCCCTGTCCCGTG |
| | | | ACAGTGTCCTGGAACTCTGGGGCTCTGACTTCCGGCGT |
| | | | GCACACCTTCCCCGCCGTGCTGCAGAGCAGCGGCCTGT |
| | | | ACAGCCTGAGCAGCGTGGTGACAGTGCCCTCCAGCTCT |
| | | | CTGGGAACCCAGACCTATATCTGCAACGTGAACCACAA |
| | | | GCCCAGCAACACCAAGGTGGACAAGAGAGTGGAGCCCA |
| | | | AGAGCTGCGACAAGACCCACACCTGCCCCCCCTGCCCA |
| | | | GCTCCAGAACTGCTGGGAGGGCCTTCCGTGTTCCTGTT |
| | | | CCCCCCCAAGCCCAAGGACACCCTGATGATCAGCAGGA |
| | | | CCCCCGAGGTGACCTGCGTGGTGGTGGACGTGTCCCAC |
| | | | GAGGACCCAGAGGTGAAGTTCAACTGGTACGTGGACGG |
| | | | CGTGGAGGTGCACAACGCCAAGACCAAGCCCAGAGAGG |
| | | | AGCAGTACAACAGCACCTACAGGGTGGTGTCCGTGCTG |
| | | | ACCGTGCTGCACCAGGACTGGCTGAACGGCAAAGAATA |
| | | | CAAGTGCAAAGTCTCCAACAAGGCCCTGCCAGCCCCAA |
| | | | TCGAAAAGACAATCAGCAAGGCCAAGGGCCAGCCACGG |
| | | | GAGCCCCAGGTGTACACCCTGCCCCCCAGCCGGGAGGA |
| | | | GATGACCAAGAACCAGGTGTCCCTGACCTGTCTGGTGA |
| | | | AGGGCTTCTACCCCTGTGATATCGCCGTGGAGTGGGAG |
| | | | AGCAACGGCCAGCCCGAGAACAACTACAAGACCACCCC |
| | | | CCCAGTGCTGGACAGCGACGGCAGCTTCTTCCTGTACA |
| | | | GCAAGCTGACCGTGGACAAGTCCAGGTGGCAGCAGGGC |
| | | | AACGTGTTCAGCTGCAGCGTGATGCACGAGGCCCTGCA |
| | | | CAACCACTACACCCAGAAGTCCCTGAGCCTGAGCCCCG |
| | | | GCAAG |
| | 379 | Light Chain | DIQMTQSPSSVSASVGDRVTITCKASQDVGTAVAWYQQ |
| | | | KPGKAPKLLIYWASIRHTGVPSRFSGSGSGTDFTLTIS |
| | | | SLQPEDFATYYCHQFATYTFGGGTKVEIKRTVAAPSVF |
| | | | IFPPSDEQLKSGTASVVCLLNNFYPREAKVQWKVDNAL |
| | | | QSGNSQESVTEQDSKDSTYSLSSTLTLSKADYEKHKVY |
| | | | ACEVTHQGLSSPVTKSFNRGEC |
| | 380 | DNA Light | GACATTCAGATGACACAGAGCCCTAGCAGCGTGTCCGC |
| | | Chain | CTCTGTGGGAGACAGAGTGACCATCACATGCAAGGCCA |
| | | | GCCAGGATGTGGGAACAGCCGTGGCTTGGTATCAGCAG |
| | | | AAGCCTGGCAAGGCCCCTAAGCTGCTGATCTACTGGGC |
| | | | CAGCATCAGACACACAGGCGTGCCCAGCAGATTTTCTG |
| | | | GCAGCGGCTCTGGCACCGACTTCACCCTGACCATATCT |
| | | | AGCCTGCAGCCAGAGGACTTCGCCACCTACTACTGCCA |
| | | | CCAGTTTGCCACCTACACCTTCGGCGGAGGCACCAAGG |
| | | | TGGAAATCAAGCGTACGGTGGCCGCTCCCAGCGTGTTC |
| | | | ATCTTCCCCCCCAGCGACGAGCAGCTGAAGAGTGGCAC |
| | | | CGCCAGCGTGGTGTGCCTGCTGAACAACTTCTACCCCC |
| | | | GGGAGGCCAAGGTGCAGTGGAAGGTGGACAACGCCCTG |
| | | | CAGAGCGGCAACAGCCAGGAGAGCGTCACCGAGCAGGA |
| | | | CAGCAAGGACTCCACCTACAGCCTGAGCAGCACCCTGA |
| | | | CCCTGAGCAAGGCCGACTACGAGAAGCATAAGGTGTAC |
| | | | GCCTGCGAGGTGACCCACCAGGGCCTGTCCAGCCCCGT |
| | | | GACCAAGAGCTTCAACAGGGGCGAGTGC |
| 5T4 | 381 | Heavy | EVQLVESGGGLVQPGGSLRLSCAASGYTFTNFGMNWVR |
| Cysmab | | Chain (Wild | QAPGKGLEWVAWINTNTGEPRYAEEFKGRFTISRDNAK |
| | | Type Fc) | NSLYLQMNSLRAEDTAVYYCARDWDGAYFFDYWGQGTL |
| | | | VTVSSASTKGPSVFPLAPSSKSTSGGTAALGCLVKDYF |
| | | | PCPVTVSWNSGALTSGVHTFPAVLQSSGLYSLSSVVTV |
| | | | PSSSLGTQTYICNVNHKPSNTKVDKRVEPKSCDKTHTC |
| | | | PPCPAPELLGGPSVFLFPPKPKDTLMISRTPEVTCVVV |
| | | | DVSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSREEMTKNQVSLTCLVKGFYPCDIA |
| | | | VEWESNGQPENNYKTTPPVLDSDGSFFLYSKLTVDKSR |
| | | | WQQGNVFSCSVMHEALHNHYTQKSLSLSPGK |
| | 382 | DNA Heavy | GAAGTGCAGCTGGTTGAATCTGGCGGAGGACTGGTTCA |
| | | Chain (Wild | GCCTGGCGGATCTCTGAGACTGTCTTGTGCCGCCAGCG |
| | | Type Fc) | GCTACACCTTCACCAACTTCGGCATGAACTGGGTCCGA |
| | | | CAGGCCCCTGGCAAAGGCCTTGAATGGGTCGCCTGGAT |
| | | | CAACACCAATACCGGCGAGCCCAGATACGCCGAAGAGT |
| | | | TCAAGGGCAGATTCACCATCAGCCGGGACAACGCCAAG |
| | | | AACAGCCTGTACCTGCAGATGAACTCCCTGAGAGCCGA |
| | | | GGACACCGCCGTGTACTACTGTGCCAGAGATTGGGACG |
| | | | GCGCCTACTTCTTCGATTACTGGGGCCAGGGCACCCTG |
| | | | GTCACAGTTAGTTCTGCTAGCACCAAGGGCCCAAGTGT |
| | | | GTTTCCCCTGGCCCCCAGCAGCAAGTCTACTTCCGGCG |
| | | | GAACTGCTGCCCTGGGTTGCCTGGTGAAGGACTACTTC |
| | | | CCCTGTCCCGTGACAGTGTCCTGGAACTCTGGGGCTCT |
| | | | GACTTCCGGCGTGCACACCTTCCCCGCCGTGCTGCAGA |
| | | | GCAGCGGCCTGTACAGCCTGAGCAGCGTGGTGACAGTG |
| | | | CCCTCCAGCTCTCTGGGAACCCAGACCTATATCTGCAA |
| | | | CGTGAACCACAAGCCCAGCAACACCAAGGTGGACAAGA |
| | | | GAGTGGAGCCCAAGAGCTGCGACAAGACCCACACCTGC |
| | | | CCCCCCTGCCCAGCTCCAGAACTGCTGGGAGGGCCTTC |
| | | | CGTGTTCCTGTTCCCCCCCAAGCCCAAGGACACCCTGA |
| | | | TGATCAGCAGGACCCCCGAGGTGACCTGCGTGGTGGTG |
| | | | GACGTGTCCCACGAGGACCCAGAGGTGAAGTTCAACTG |
| | | | GTACGTGGACGGCGTGGAGGTGCACAACGCCAAGACCA |
| | | | AGCCCAGAGAGGAGCAGTACAACAGCACCTACAGGGTG |
| | | | GTGTCCGTGCTGACCGTGCTGCACCAGGACTGGCTGAA |
| | | | CGGCAAAGAATACAAGTGCAAAGTCTCCAACAAGGCCC |
| | | | TGCCAGCCCCAATCGAAAAGACAATCAGCAAGGCCAAG |
| | | | GGCCAGCCACGGGAGCCCCAGGTGTACACCCTGCCCCC |
| | | | CAGCCGGGAGGAGATGACCAAGAACCAGGTGTCCCTGA |
| | | | CCTGTCTGGTGAAGGGCTTCTACCCCTGTGATATCGCC |
| | | | GTGGAGTGGGAGAGCAACGGCCAGCCCGAGAACAACTA |
| | | | CAAGACCACCCCCCCAGTGCTGGACAGCGACGGCAGCT |
| | | | TCTTCCTGTACAGCAAGCTGACCGTGGACAAGTCCAGG |
| | | | TGGCAGCAGGGCAACGTGTTCAGCTGCAGCGTGATGCA |
| | | | CGAGGCCCTGCACAACCACTACACCCAGAAGTCCCTGA |
| | | | GCCTGAGCCCCGGCAAG |
| 5T4 DANAPA | 383 | Heavy | EVQLVESGGGLVQPGGSLRLSCAASGYTFTNFGMNWVR |
| Cysmab | | Chain (Fc | QAPGKGLEWVAWINTNTGEPRYAEEFKGRFTISRDNAK |
| | | Silenced | NSLYLQMNSLRAEDTAVYYCARDWDGAYFFDYWGQGTL |
| | | DANAPA) | VTVSSASTKGPSVFPLAPSSKSTSGGTAALGCLVKDYF |
| | | | PCPVTVSWNSGALTSGVHTFPAVLQSSGLYSLSSVVTV |
| | | | PSSSLGTQTYICNVNHKPSNTKVDKRVEPKSCDKTHTC |
| | | | PPCPAPELLGGPSVFLFPPKPKDTLMISRTPEVTCVVV |
| | | | AVSHEDPEVKFNWYVDGVEVHNAKTKPREEQYASTYRV |
| | | | VSVLTVLHQDWLNGKEYKCKVSNKALAAPIEKTISKAK |
| | | | GQPREPQVYTLPPSREEMTKNQVSLTCLVKGFYPCDIA |
| | | | VEWESNGQPENNYKTTPPVLDSDGSFFLYSKLTVDKSR |
| | | | WQQGNVFSCSVMHEALHNHYTQKSLSLSPGK |
| | 384 | DNA Heavy | GAAGTGCAGCTGGTTGAATCTGGCGGAGGACTGGTTCA |
| | | Chain (Fc | GCCTGGCGGATCTCTGAGACTGTCTTGTGCCGCCAGCG |
| | | Silenced | GCTACACCTTCACCAACTTCGGCATGAACTGGGTCCGA |
| | | DANAPA) | CAGGCCCCTGGCAAAGGCCTTGAATGGGTCGCCTGGAT |
| | | | CAACACCAATACCGGCGAGCCCAGATACGCCGAAGAGT |
| | | | TCAAGGGCAGATTCACCATCAGCCGGGACAACGCCAAG |
| | | | AACAGCCTGTACCTGCAGATGAACTCCCTGAGAGCCGA |
| | | | GGACACCGCCGTGTACTACTGTGCCAGAGATTGGGACG |
| | | | GCGCCTACTTCTTCGATTACTGGGGCCAGGGCACCCTG |
| | | | GTCACAGTTAGTTCTGCTAGCACCAAGGGCCCAAGTGT |
| | | | GTTTCCCCTGGCCCCCAGCAGCAAGTCTACTTCCGGCG |
| | | | GAACTGCTGCCCTGGGTTGCCTGGTGAAGGACTACTTC |
| | | | CCCTGTCCCGTGACAGTGTCCTGGAACTCTGGGGCTCT |
| | | | GACTTCCGGCGTGCACACCTTCCCCGCCGTGCTGCAGA |
| | | | GCAGCGGCCTGTACAGCCTGAGCAGCGTGGTGACAGTG |
| | | | CCCTCCAGCTCTCTGGGAACCCAGACCTATATCTGCAA |
| | | | CGTGAACCACAAGCCCAGCAACACCAAGGTGGACAAGA |
| | | | GAGTGGAGCCCAAGAGCTGCGACAAGACCCACACCTGC |
| | | | CCCCCCTGCCCAGCTCCAGAACTGCTGGGAGGGCCTTC |
| | | | CGTGTTCCTGTTCCCCCCCAAGCCCAAGGACACCCTGA |
| | | | TGATCAGCAGGACCCCCGAGGTGACCTGCGTGGTGGTG |
| | | | GCCGTGTCCCACGAGGACCCAGAGGTGAAGTTCAACTG |
| | | | GTACGTGGACGGCGTGGAGGTGCACAACGCCAAGACCA |
| | | | AGCCCAGAGAGGAGCAGTACGCCAGCACCTACAGGGTG |
| | | | GTGTCCGTGCTGACCGTGCTGCACCAGGACTGGCTGAA |
| | | | CGGCAAAGAATACAAGTGCAAAGTCTCCAACAAGGCCC |
| | | | TGGCTGCCCCAATCGAAAAGACAATCAGCAAGGCCAAG |
| | | | GGCCAGCCACGGGAGCCCCAGGTGTACACCCTGCCCCC |
| | | | CAGCCGGGAGGAGATGACCAAGAACCAGGTGTCCCTGA |
| | | | CCTGTCTGGTGAAGGGCTTCTACCCCTGTGATATCGCC |
| | | | GTGGAGTGGGAGAGCAACGGCCAGCCCGAGAACAACTA |
| | | | CAAGACCACCCCCCCAGTGCTGGACAGCGACGGCAGCT |
| | | | TCTTCCTGTACAGCAAGCTGACCGTGGACAAGTCCAGG |
| | | | TGGCAGCAGGGCAACGTGTTCAGCTGCAGCGTGATGCA |
| | | | CGAGGCCCTGCACAACCACTACACCCAGAAGTCCCTGA |
| | | | GCCTGAGCCCCGGCAAG |
| 5T4 LC | 385 | Light Chain | DIQMTQSPSSLSASVGDRVTITCKASQSVSNDVAWYQQ |
| | | | KPGKAPKLLIYFATNRYTGVPSRFSGSGYGTDFTLTIS |
| | | | SLQPEDFATYYCQQDYSSPWTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 386 | DNA Light | GACATTCAGATGACACAGAGCCCTAGCAGCCTGAGCGC |
| | | Chain | CTCTGTGGGAGACAGAGTGACCATCACATGCAAGGCCA |
| | | | GCCAGAGCGTGTCCAACGACGTGGCATGGTATCAGCAG |
| | | | AAGCCTGGCAAGGCCCCTAAGCTGCTGATCTACTTCGC |
| | | | CACCAACAGATACACCGGCGTGCCCAGCAGATTTTCCG |
| | | | GCTCTGGCTACGGCACCGACTTCACCCTGACCATATCT |
| | | | AGCCTGCAGCCAGAGGATTTCGCCACCTACTACTGCCA |
| | | | GCAGGACTACAGCAGCCCTTGGACATTTGGCCAGGGAA |
| | | | CAAAGGTGGAAATCAAGCGTACGGTGGCCGCTCCCAGC |
| | | | GTGTTCATCTTCCCCCCCAGCGACGAGCAGCTGAAGAG |
| | | | TGGCACCGCCAGCGTGGTGTGCCTGCTGAACAACTTCT |
| | | | ACCCCCGGGAGGCCAAGGTGCAGTGGAAGGTGGACAAC |
| | | | GCCCTGCAGAGCGGCAACAGCCAGGAGAGCGTCACCGA |
| | | | GCAGGACAGCAAGGACTCCACCTACAGCCTGAGCAGCA |
| | | | CCCTGACCCTGAGCAAGGCCGACTACGAGAAGCATAAG |
| | | | GTGTACGCCTGCGAGGTGACCCACCAGGGCCTGTCCAG |
| | | | CCCCGTGACCAAGAGCTTCAACAGGGGCGAGTGC |
| Trop2 | 387 | Heavy | QVQLVQSGAEVKKPGASVKVSCKASGYTFTTAGMQWVR |
| Datopotamab | | Chain (Wild | QAPGQGLEWMGWINTHSGVPKYAEDFKGRVTISADTST |
| Cysmab | | Type Fc) | STAYLQLSSLKSEDTAVYYCARSGFGSSYWYFDVWGQG |
| | | | TLVTVSSASTKGPSVFPLAPSSKSTSGGTAALGCLVKD |
| | | | YFPCPVTVSWNSGALTSGVHTFPAVLQSSGLYSLSSVV |
| | | | TVPSSSLGTQTYICNVNHKPSNTKVDKRVEPKSCDKTH |
| | | | TCPPCPAPELLGGPSVFLFPPKPKDTLMISRTPEVTCV |
| | | | VVDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSREEMTKNQVSLTCLVKGFYPCD |
| | | | IAVEWESNGQPENNYKTTPPVLDSDGSFFLYSKLTVDK |
| | | | SRWQQGNVFSCSVMHEALHNHYTQKSLSLSPGK |
| | 388 | DNA Heavy | CAGGTTCAGCTGGTTCAGTCTGGCGCCGAAGTGAAGAA |
| | | Chain (Wild | ACCTGGCGCCTCTGTGAAGGTGTCCTGCAAGGCCAGCG |
| | | Type Fc) | GCTACACCTTTACAACAGCCGGCATGCAGTGGGTCCGA |
| | | | CAGGCTCCTGGACAAGGCTTGGAATGGATGGGCTGGAT |
| | | | CAACACCCACAGCGGCGTGCCCAAATACGCCGAGGATT |
| | | | TCAAGGGCAGAGTGACCATCAGCGCCGACACCAGCACA |
| | | | AGCACAGCCTACCTGCAGCTGAGCAGCCTGAAGTCTGA |
| | | | GGACACCGCCGTGTACTACTGTGCCAGAAGCGGCTTTG |
| | | | GCAGCAGCTACTGGTACTTCGATGTGTGGGGCCAGGGC |
| | | | ACCCTGGTCACAGTTAGTTCTGCTAGCACCAAGGGCCC |
| | | | AAGTGTGTTTCCCCTGGCCCCCAGCAGCAAGTCTACTT |
| | | | CCGGCGGAACTGCTGCCCTGGGTTGCCTGGTGAAGGAC |
| | | | TACTTCCCCTGTCCCGTGACAGTGTCCTGGAACTCTGG |
| | | | GGCTCTGACTTCCGGCGTGCACACCTTCCCCGCCGTGC |
| | | | TGCAGAGCAGCGGCCTGTACAGCCTGAGCAGCGTGGTG |
| | | | ACAGTGCCCTCCAGCTCTCTGGGAACCCAGACCTATAT |
| | | | CTGCAACGTGAACCACAAGCCCAGCAACACCAAGGTGG |
| | | | ACAAGAGAGTGGAGCCCAAGAGCTGCGACAAGACCCAC |
| | | | ACCTGCCCCCCCTGCCCAGCTCCAGAACTGCTGGGAGG |
| | | | GCCTTCCGTGTTCCTGTTCCCCCCCAAGCCCAAGGACA |
| | | | CCCTGATGATCAGCAGGACCCCCGAGGTGACCTGCGTG |
| | | | GTGGTGGACGTGTCCCACGAGGACCCAGAGGTGAAGTT |
| | | | CAACTGGTACGTGGACGGCGTGGAGGTGCACAACGCCA |
| | | | AGACCAAGCCCAGAGAGGAGCAGTACAACAGCACCTAC |
| | | | AGGGTGGTGTCCGTGCTGACCGTGCTGCACCAGGACTG |
| | | | GCTGAACGGCAAAGAATACAAGTGCAAAGTCTCCAACA |
| | | | AGGCCCTGCCAGCCCCAATCGAAAAGACAATCAGCAAG |
| | | | GCCAAGGGCCAGCCACGGGAGCCCCAGGTGTACACCCT |
| | | | GCCCCCCAGCCGGGAGGAGATGACCAAGAACCAGGTGT |
| | | | CCCTGACCTGTCTGGTGAAGGGCTTCTACCCCTGTGAT |
| | | | ATCGCCGTGGAGTGGGAGAGCAACGGCCAGCCCGAGAA |
| | | | CAACTACAAGACCACCCCCCCAGTGCTGGACAGCGACG |
| | | | GCAGCTTCTTCCTGTACAGCAAGCTGACCGTGGACAAG |
| | | | TCCAGGTGGCAGCAGGGCAACGTGTTCAGCTGCAGCGT |
| | | | GATGCACGAGGCCCTGCACAACCACTACACCCAGAAGT |
| | | | CCCTGAGCCTGAGCCCCGGCAAG |
| Trop2 | 389 | Heavy | QVQLVQSGAEVKKPGASVKVSCKASGYTFTTAGMQWVR |
| Datopotamab | | Chain (Fc | QAPGQGLEWMGWINTHSGVPKYAEDFKGRVTISADTST |
| DANAPA | | Silenced | STAYLQLSSLKSEDTAVYYCARSGFGSSYWYFDVWGQG |
| Cysmab | | DANAPA) | TLVTVSSASTKGPSVFPLAPSSKSTSGGTAALGCLVKD |
| | | | YFPCPVTVSWNSGALTSGVHTFPAVLQSSGLYSLSSVV |
| | | | TVPSSSLGTQTYICNVNHKPSNTKVDKRVEPKSCDKTH |
| | | | TCPPCPAPELLGGPSVFLFPPKPKDTLMISRTPEVTCV |
| | | | VVAVSHEDPEVKFNWYVDGVEVHNAKTKPREEQYASTY |
| | | | RVVSVLTVLHQDWLNGKEYKCKVSNKALAAPIEKTISK |
| | | | AKGQPREPQVYTLPPSREEMTKNQVSLTCLVKGFYPCD |
| | | | IAVEWESNGQPENNYKTTPPVLDSDGSFFLYSKLTVDK |
| | | | SRWQQGNVFSCSVMHEALHNHYTQKSLSLSPGK |
| | 390 | DNA Heavy | CAGGTTCAGCTGGTTCAGTCTGGCGCCGAAGTGAAGAA |
| | | Chain (Fc | ACCTGGCGCCTCTGTGAAGGTGTCCTGCAAGGCCAGCG |
| | | Silenced | GCTACACCTTTACAACAGCCGGCATGCAGTGGGTCCGA |
| | | DANAPA) | CAGGCTCCTGGACAAGGCTTGGAATGGATGGGCTGGAT |
| | | | CAACACCCACAGCGGCGTGCCCAAATACGCCGAGGATT |
| | | | TCAAGGGCAGAGTGACCATCAGCGCCGACACCAGCACA |
| | | | AGCACAGCCTACCTGCAGCTGAGCAGCCTGAAGTCTGA |
| | | | GGACACCGCCGTGTACTACTGTGCCAGAAGCGGCTTTG |
| | | | GCAGCAGCTACTGGTACTTCGATGTGTGGGGCCAGGGC |
| | | | ACCCTGGTCACAGTTAGTTCTGCTAGCACCAAGGGCCC |
| | | | AAGTGTGTTTCCCCTGGCCCCCAGCAGCAAGTCTACTT |
| | | | CCGGCGGAACTGCTGCCCTGGGTTGCCTGGTGAAGGAC |
| | | | TACTTCCCCTGTCCCGTGACAGTGTCCTGGAACTCTGG |
| | | | GGCTCTGACTTCCGGCGTGCACACCTTCCCCGCCGTGC |
| | | | TGCAGAGCAGCGGCCTGTACAGCCTGAGCAGCGTGGTG |
| | | | ACAGTGCCCTCCAGCTCTCTGGGAACCCAGACCTATAT |
| | | | CTGCAACGTGAACCACAAGCCCAGCAACACCAAGGTGG |
| | | | ACAAGAGAGTGGAGCCCAAGAGCTGCGACAAGACCCAC |
| | | | ACCTGCCCCCCCTGCCCAGCTCCAGAACTGCTGGGAGG |
| | | | GCCTTCCGTGTTCCTGTTCCCCCCCAAGCCCAAGGACA |
| | | | CCCTGATGATCAGCAGGACCCCCGAGGTGACCTGCGTG |
| | | | GTGGTGGCCGTGTCCCACGAGGACCCAGAGGTGAAGTT |
| | | | CAACTGGTACGTGGACGGCGTGGAGGTGCACAACGCCA |
| | | | AGACCAAGCCCAGAGAGGAGCAGTACGCCAGCACCTAC |
| | | | AGGGTGGTGTCCGTGCTGACCGTGCTGCACCAGGACTG |
| | | | GCTGAACGGCAAAGAATACAAGTGCAAAGTCTCCAACA |
| | | | AGGCCCTGGCTGCCCCAATCGAAAAGACAATCAGCAAG |
| | | | GCCAAGGGCCAGCCACGGGAGCCCCAGGTGTACACCCT |
| | | | GCCCCCCAGCCGGGAGGAGATGACCAAGAACCAGGTGT |
| | | | CCCTGACCTGTCTGGTGAAGGGCTTCTACCCCTGTGAT |
| | | | ATCGCCGTGGAGTGGGAGAGCAACGGCCAGCCCGAGAA |
| | | | CAACTACAAGACCACCCCCCCAGTGCTGGACAGCGACG |
| | | | GCAGCTTCTTCCTGTACAGCAAGCTGACCGTGGACAAG |
| | | | TCCAGGTGGCAGCAGGGCAACGTGTTCAGCTGCAGCGT |
| | | | GATGCACGAGGCCCTGCACAACCACTACACCCAGAAGT |
| | | | CCCTGAGCCTGAGCCCCGGCAAG |
| Trop2 | 391 | Light Chain | DIQMTQSPSSLSASVGDRVTITCKASQDVSTAVAWYQQ |
| Datopotamab | | | KPGKAPKLLIYSASYRYTGVPSRFSGSGSGTDFTLTIS |
| LC | | | SLQPEDFAVYYCQQHYITPLTFGQGTKLEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 392 | DNA Light | GACATTCAGATGACACAGAGCCCTAGCAGCCTGAGCGC |
| | | Chain | CTCTGTGGGAGACAGAGTGACCATCACATGCAAGGCCA |
| | | | GCCAGGATGTGTCTACAGCCGTGGCCTGGTATCAGCAG |
| | | | AAGCCTGGAAAGGCCCCTAAGCTGCTGATCTACAGCGC |
| | | | CAGCTACAGATACACCGGCGTGCCCAGCAGATTTTCTG |
| | | | GCAGCGGCTCTGGCACCGACTTCACCCTGACCATATCT |
| | | | AGCCTGCAGCCAGAGGACTTCGCCGTGTACTACTGCCA |
| | | | GCAGCACTACATCACCCCTCTGACCTTTGGCCAGGGGA |
| | | | CCAAGCTGGAAATCAAGCGTACGGTGGCCGCTCCCAGC |
| | | | GTGTTCATCTTCCCCCCCAGCGACGAGCAGCTGAAGAG |
| | | | TGGCACCGCCAGCGTGGTGTGCCTGCTGAACAACTTCT |
| | | | ACCCCCGGGAGGCCAAGGTGCAGTGGAAGGTGGACAAC |
| | | | GCCCTGCAGAGCGGCAACAGCCAGGAGAGCGTCACCGA |
| | | | GCAGGACAGCAAGGACTCCACCTACAGCCTGAGCAGCA |
| | | | CCCTGACCCTGAGCAAGGCCGACTACGAGAAGCATAAG |
| | | | GTGTACGCCTGCGAGGTGACCCACCAGGGCCTGTCCAG |
| | | | CCCCGTGACCAAGAGCTTCAACAGGGGCGAGTGCTGA |

TABLE D8
Exemplary Antibody CDR and Variable Region Sequences

| | SEQ ID | | |
|---|---|---|---|
| Ab | NO | IgG chain | Amino acid sequence |
| CD74 | 153 | VH (Wild | QVQLQQSGSELKKPGASVKVSCKASGYTFTNYGVNWI |
| milatuzumab | | Type Fc) | KQAPGQGLQWMGWINPNTGEPTFDDDFKGRFAFSLDT |
| Cysmab HC | | | SVSTAYLQISSLKADDTAVYFCSRSRGKNEAWFAYWG |
| (VHmil x | | | QGTLVTVSS |
| VK1aNQ) | | | |
| HCDR1 | 256 | Chothia | GYTFTNY |
| HCDR2 | 257 | Chothia | NPNTGE |
| HCDR3 | 171 | Chothia | SRGKNEAWFAY |
| HCDR1 | 258 | Kabat | NYGVN |
| HCDR2 | 170 | Kabat | WINPNTGEPTFDDDFKG |
| HCDR3 | 171 | Kabat | SRGKNEAWFAY |
| HCDR1 | 259 | IMGT | GYTFTNYG |
| HCDR2 | 260 | IMGT | INPNTGEP |
| HCDR3 | 261 | IMGT | SRSRGKNEAWFAY |
| HCDR1 | 169 | Combined | GYTFTNYGVN |
| HCDR2 | 170 | Combined | WINPNTGEPTFDDDFKG |
| HCDR3 | 171 | Combined | SRGKNEAWFAY |
| CD74 | 153 | Heavy | QVQLQQSGSELKKPGASVKVSCKASGYTFTNYGVNWI |
| Milatuzumab | | Chain (Fc | KQAPGQGLQWMGWINPNTGEPTFDDDFKGRFAFSLDT |
| DANAPA | | Silenced | SVSTAYLQISSLKADDTAVYFCSRSRGKNEAWFAYWG |
| Cysmab | | DANAPA) | QGTLVTVSS |
| HCDR1 | 256 | Chothia | GYTFTNY |
| HCDR2 | 257 | Chothia | NPNTGE |
| HCDR3 | 171 | Chothia | SRGKNEAWFAY |
| HCDR1 | 258 | Kabat | NYGVN |
| HCDR2 | 170 | Kabat | WINPNTGEPTFDDDFKG |
| HCDR3 | 171 | Kabat | SRGKNEAWFAY |
| HCDR1 | 259 | IMGT | GYTFTNYG |
| HCDR2 | 260 | IMGT | INPNTGEP |
| HCDR3 | 261 | IMGT | SRSRGKNEAWFAY |
| HCDR1 | 169 | Combined | GYTFTNYGVN |
| HCDR2 | 170 | Combined | WINPNTGEPTFDDDFKG |
| HCDR3 | 171 | Combined | SRGKNEAWFAY |
| CD74 | 262 | Light Chain | DIQLTQSPLSLPVTLGQPASISCRSSQSLVHRNGNTY |
| Milatuzumab LC | | | LHWFQQRPGQSPRLLIYTVSNRFSGVPDRFSGSGSGT |
| | | | DFTLKISRVEAEDVGVYFCSQSSHVPPTFGAGTRLEI |
| | | | K |
| LCDR1 | 263 | Chothia | SQSLVHRNGNTY |
| LCDR2 | 264 | Chothia | TVS |
| LCDR3 | 265 | Chothia | SSHVPP |
| LCDR1 | 266 | Kabat | RSSQSLVHRNGNTYLH |
| LCDR2 | 173 | Kabat | TVSNRFS |
| LCDR3 | 174 | Kabat | SQSSHVPPT |
| LCDR1 | 215 | IMGT | QSLVHRNGNTY |
| LCDR2 | 264 | IMGT | TVS |
| LCDR3 | 174 | IMGT | SQSSHVPPT |
| LCDR1 | 266 | Combined | RSSQSLVHRNGNTYLH |
| LCDR2 | 173 | Combined | TVSNRFS |
| LCDR3 | 174 | Combined | SQSSHVPPT |
| CD74 LC | 267 | Light Chain | DIVMTQTPLSLPVTPGEPASISCRSSQSLVHRNQNTY |
| Cysmab WT | | | LHWYLQKPGQSPQLLIYTVSNRFSGVPDRFSGSGSGT |
| (VHmil x | | | DFTLKSRVEAEDVGVYFCSQSSHVPPTFGQGTKLEIK |
| VK1aNQ) | | | |
| LCDR1 | 268 | Chothia | SQSLVHRNQNTY |
| LCDR2 | 264 | Chothia | TVS |
| LCDR3 | 265 | Chothia | SSHVPP |
| LCDR1 | 172 | Kabat | RSSQSLVHRNQNTYLH |
| LCDR2 | 173 | Kabat | TVSNRFS |
| LCDR3 | 174 | Kabat | SQSSHVPPT |
| LCDR1 | 269 | IMGT | QSLVHRNQNTY |
| LCDR2 | 264 | IMGT | TVS |
| LCDR3 | 174 | IMGT | SQSSHVPPT |
| LCDR1 | 172 | Combined | RSSQSLVHRNQNTYLH |
| LCDR2 | 173 | Combined | TVSNRFS |
| LCDR3 | 174 | Combined | SQSSHVPPT |
| CD48 NY920 | 270 | Heavy | EVQLLESGGGLVQPGGSLRLSCAASGFTFSSFAMSWV |
| Cysmab WT HC | | Chain | RQAPGKGLEWVSAISGFGGSTYYADSVKGRFTISRDN |
| | | (Wild Type | SKNTLYLQMNSLRAEDTAVYYCARQFWEDQPFYFDYW |
| | | Fc) | GQGTLVTVSS |
| HCDR1 | 271 | Chothia | GFTFSSF |
| HCDR2 | 272 | Chothia | SGFGGS |
| HCDR3 | 273 | Chothia | QFWEDQPFYFDY |
| HCDR1 | 274 | Kabat | SFAMS |
| HCDR2 | 275 | Kabat | AISGFGGSTYYADSVKG |
| HCDR3 | 273 | Kabat | QFWEDQPFYFDY |
| HCDR1 | 276 | IMGT | GFTFSSFA |
| HCDR2 | 277 | IMGT | ISGFGGST |
| HCDR3 | 278 | IMGT | ARQFWEDQPFYFDY |
| HCDR1 | 279 | Combined | GFTFSSFAMS |
| HCDR2 | 275 | Combined | AISGFGGSTYYADSVKG |
| HCDR3 | 273 | Combined | QFWEDQPFYFDY |
| CD48 NY920 | 270 | | EVQLLESGGGLVQPGGSLRLSCAASGFTFSSFAMSWV |
| Cysmab DAPA | | | RQAPGKGLEWVSAISGFGGSTYYADSVKGRFTISRDN |
| HC | | | SKNTLYLQMNSLRAEDTAVYYCARQFWEDQPFYFDYW |
| | | | GQGTLVTVSS |
| HCDR1 | 271 | Chothia | GFTFSSF |
| HCDR2 | 272 | Chothia | SGFGGS |
| HCDR3 | 273 | Chothia | QFWEDQPFYFDY |
| HCDR1 | 274 | Kabat | SFAMS |
| HCDR2 | 275 | Kabat | AISGFGGSTYYADSVKG |
| HCDR3 | 273 | Kabat | QFWEDQPFYFDY |
| HCDR1 | 276 | IMGT | GFTFSSFA |
| HCDR2 | 277 | IMGT | ISGFGGST |
| HCDR3 | 278 | IMGT | ARQFWEDQPFYFDY |
| HCDR1 | 279 | Combined | GFTFSSFAMS |
| HCDR2 | 275 | Combined | AISGFGGSTYYADSVKG |
| HCDR3 | 273 | Combined | QFWEDQPFYFDY |
| CD48 NY920 | 280 | Light Chain | DIQMTQSPSSLSASVGDRVTITCRASQSISSYLNWYQ |
| LC | | | QKPGKAPKLLIYAASSLQSGVPSRFSGSGSGTDFTLT |
| | | | ISSLQPEDFATYYCQQSYSTPLTFGQGTKVEIK |
| LCDR1 | 281 | Chothia | SQSISSY |
| LCDR2 | 282 | Chothia | AAS |
| LCDR3 | 283 | Chothia | SYSTPL |
| LCDR1 | 284 | Kabat | RASQSISSYLN |
| LCDR2 | 285 | Kabat | AASSLQS |
| LCDR3 | 286 | Kabat | QQSYSTPLT |
| LCDR1 | 287 | IMGT | QSISSY |
| LCDR2 | 282 | IMGT | AAS |
| LCDR3 | 286 | IMGT | QQSYSTPLT |
| LCDR1 | 284 | Combined | RASQSISSYLN |
| LCDR2 | 288 | Combined | AASSLQS |
| LCDR3 | 286 | Combined | QQSYSTPLT |
| Her2 Cysmab | 9 | Heavy | EVQLVESGGGLVQPGGSLRLSCAASGFNIKDTYIHWV |
| WT HC | | Chain | RQAPGKGLEWVARIYPTNGYTRYADSVKGRFTISADT |
| | | (Wild Type | SKNTAYLQMNSLRAEDTAVYYCSRWGGDGFYAMDYWG |
| | | Fc) | QGTLVTVSS |
| HCDR1 | 289 | Chothia | GFNIKDT |
| HCDR2 | 290 | Chothia | YPTNGY |
| HCDR3 | 291 | Chothia | WGGDGFYAMDY |
| HCDR1 | 292 | Kabat | DTYIH |
| HCDR2 | 40 | Kabat | RIYPTNGYTRYADSVKG |
| HCDR3 | 291 | Kabat | WGGDGFYAMDY |
| HCDR1 | 293 | IMGT | GFNIKDTY |
| HCDR2 | 294 | IMGT | IYPTNGYT |
| HCDR3 | 295 | IMGT | SRWGGDGFYAMDY |
| HCDR1 | 39 | Combined | GFNIKDTYIH |
| HCDR2 | 40 | Combined | RIYPTNGYTRYADSVKG |
| HCDR3 | 291 | Combined | WGGDGFYAMDY |
| Her2 LC | 296 | Light Chain | DIQMTQSPSSLSASVGDRVTITCRASQDVNTAVAWYQ |
| | | | QKPGKAPKLLIYSASFLYSGVPSRFSGSRSGTDFTLT |
| | | | ISSLQPEDFATYYCQQHYTTPPTFGQGTKVEIK |
| LCDR1 | 297 | Chothia | SQDVNTA |
| LCDR2 | 298 | Chothia | SAS |
| LCDR3 | 299 | Chothia | HYTTPP |
| LCDR1 | 300 | Kabat | RASQDVNTAVA |
| LCDR2 | 301 | Kabat | SASFLYS |
| LCDR3 | 44 | Kabat | QQHYTTPPT |
| LCDR1 | 302 | IMGT | QDVNTA |
| LCDR2 | 298 | IMGT | SAS |
| LCDR3 | 44 | IMGT | QQHYTTPPT |
| LCDR1 | 300 | Combined | RASQDVNTAVA |
| LCDR2 | 301 | Combined | SASFLYS |
| LCDR3 | 44 | Combined | QQHYTTPPT |
| PCAD CQY679 | 303 | Heavy | EVQLVQSGAEVKKPGESLKISCKVSGYTFTDHTIHWM |
| Cysmab WT HC | | Chain | RQMPGKGLEWMGYIYPRSGSINYNEKFKGQVTISADK |
| | | (Wild Type | SSSTAYLQWSSLKASDTAMYYCARRNLFLPMEYWGQG |
| | | Fc) | TLVTVSS |
| HCDR1 | 304 | Chothia | GYTFTDH |
| HCDR2 | 305 | Chothia | YPRSGS |
| HCDR3 | 306 | Chothia | RNLFLPMEY |
| HCDR1 | 307 | Kabat | DHTIH |
| HCDR2 | 308 | Kabat | YIYPRSGSINYNEKFKG |
| HCDR3 | 306 | Kabat | RNLFLPMEY |
| HCDR1 | 309 | IMGT | GYTFTDHT |
| HCDR2 | 277 | IMGT | ISGFGGST |
| HCDR3 | 278 | IMGT | ARQFWEDQPFYFDY |
| HCDR1 | 310 | Combined | GYTFTDHTIH |
| HCDR2 | 308 | Combined | YIYPRSGSINYNEKFKG |
| HCDR3 | 306 | Combined | RNLFLPMEY |
| PCAD CQY679 | 311 | Light Chain | DIVMTQTPLSLPVTPGEPASISCRSSQSLLSSGDQKN |
| LC | | | YLTWYLQKPGQSPQLLIYWASTRESGVPDRFSGSGSG |
| | | | TDFTLKISRVEAEDVGVYYCQNDYRYPLTFGQGTKLE |
| | | | IK |
| LCDR1 | 312 | Chothia | SQSLLSSGDQKNY |
| LCDR2 | 313 | Chothia | WAS |
| LCDR3 | 314 | Chothia | DYRYPL |
| LCDR1 | 315 | Kabat | RSSQSLLSSGDQKNYLT |
| LCDR2 | 25 | Kabat | WASTRES |
| LCDR3 | 316 | Kabat | QNDYRYPLT |
| LCDR1 | 317 | IMGT | QSLLSSGDQKNY |
| LCDR2 | 313 | IMGT | WAS |
| LCDR3 | 316 | IMGT | QNDYRYPLT |
| LCDR1 | 315 | Combined | RSSQSLLSSGDQKNYLT |
| LCDR2 | 25 | Combined | WASTRES |
| LCDR3 | 316 | Combined | QNDYRYPLT |
| EphA2 1C1 | 318 | Heavy | EVQLLESGGGLVQPGGSLRLSCAASGFTFSHYMMAWV |
| Cysmab WT HC | | Chain | RQAPGKGLEWVSRIGPSGGPTHYADSVKGRFTISRDN |
| | | (Wild Type | SKNTLYLQMNSLRAEDTAVYYCAGYDSGYDYVAVAGP |
| | | Fc) | AEYFQHWGQGTLVTVSS |
| HCDR1 | 319 | Chothia | GFTFSHY |
| HCDR2 | 320 | Chothia | GPSGGP |
| HCDR3 | 321 | Chothia | YDSGYDYVAVAGPAEYFQH |
| HCDR1 | 322 | Kabat | HYMMA |
| HCDR2 | 323 | Kabat | RIGPSGGPTHYADSVKG |
| HCDR3 | 324 | Kabat | YDSGYDYVAVAGPAEYFQH |
| HCDR1 | 325 | IMGT | GFTFSHYM |
| HCDR2 | 326 | IMGT | IGPSGGPT |
| HCDR3 | 327 | IMGT | AGYDSGYDYVAVAGPAEYFQH |
| HCDR1 | 328 | Combined | GFTFSHYMMA |
| HCDR2 | 323 | Combined | RIGPSGGPTHYADSVKG |
| HCDR3 | 321 | Combined | YDSGYDYVAVAGPAEYFQH |
| EphA2 LC | 329 | Light Chain | DIQMTQSPSSLSASVGDRVTITCRASQSISTWLAWYQ |
| | | | QKPGKAPKLLIYKASNLHTGVPSRFSGSGSGTEFSLT |
| | | | ISGLQPDDFATYYCQQYNSYSRTFGQGTKVEIK |
| LCDR1 | 330 | Chothia | SQSISTW |
| LCDR2 | 331 | Chothia | KAS |
| LCDR3 | 332 | Chothia | YNSYSR |
| LCDR1 | 333 | Kabat | RASQSISTWLA |
| LCDR2 | 334 | Kabat | KASNLHT |
| LCDR3 | 335 | Kabat | QQYNSYSRT |
| LCDR1 | 336 | IMGT | QSISTW |
| LCDR2 | 331 | IMGT | KAS |
| LCDR3 | 335 | IMGT | QQYNSYSRT |
| LCDR1 | 333 | Combined | RASQSISTWLA |
| LCDR2 | 334 | Combined | KASNLHT |
| LCDR3 | 335 | Combined | QQYNSYSRT |
| HER2 | | | |
| Disitamab | | | |
| HCDR1 | 393 | Combined | GYTFTDYYIH |
| HCDR2 | 394 | Combined | RVNPDHGDSYYNQKFKD |
| HCDR3 | 395 | Combined | NYLFDH |
| HCDR1 | 396 | Kabat | DYYIH |
| HCDR2 | 394 | Kabat | RVNPDHGDSYYNQKFKD |
| HCDR3 | 395 | Kabat | NYLFDH |
| HCDR1 | 397 | Chothia | GYTFTDY |
| HCDR2 | 398 | Chothia | NPDHGD |
| HCDR3 | 395 | Chothia | NYLFDH |
| HCDR1 | 399 | IMGT | GYTFTDYY |
| HCDR2 | 400 | IMGT | VNPDHGDS |
| HCDR3 | 401 | IMGT | ARNYLFDH |
| HER2 | | | |
| Disitamab | | | |
| LCDR1 | 402 | Combined | KASQDVGTAVA |
| LCDR2 | 403 | Combined | WASIRHT |
| LCDR3 | 404 | Combined | HQFATYT |
| LCDR1 | 402 | Kabat | KASQDVGTAVA |
| LCDR2 | 403 | Kabat | WASIRHT |
| LCDR3 | 404 | Kabat | HQFATYT |
| LCDR1 | 405 | Chothia | SQDVGTA |
| LCDR2 | 313 | Chothia | WAS |
| LCDR3 | 406 | Chothia | FATY |
| LCDR1 | 407 | IMGT | QDVGTA |
| LCDR2 | 313 | IMGT | WAS |
| LCDR3 | 404 | IMGT | HQFATYT |
| 5T4 | | | |
| HCDR1 | 408 | Combined | GYTFTNFGMN |
| HCDR2 | 409 | Combined | WINTNTGEPRYAEEFKG |
| HCDR3 | 410 | Combined | DWDGAYFFDY |
| HCDR1 | 411 | Chothia | GYTFTNF |
| HCDR2 | 412 | Chothia | NTNTGE |
| HCDR3 | 410 | Chothia | DWDGAYFFDY |
| HCDR1 | 413 | Kabat | NFGMN |
| HCDR2 | 409 | Kabat | WINTNTGEPRYAEEFKG |
| HCDR3 | 410 | Kabat | DWDGAYFFDY |
| HCDR1 | 411 | IMGT | GYTFTNFG |
| HCDR2 | 414 | IMGT | INTNTGEP |
| HCDR3 | 415 | IMGT | ARDWDGAYFFDY |
| 5T4 | | | |
| LCDR1 | 416 | Combined | KASQSVSNDVA |
| LCDR2 | 417 | Combined | FATNRYT |
| LCDR3 | 418 | Combined | QQDYSSPWT |
| LCDR1 | 419 | Chothia | SQSVSND |
| LCDR2 | 420 | Chothia | FAT |
| LCDR3 | 421 | Chothia | DYSSPW |
| LCDR1 | 416 | Kabat | KASQSVSNDVA |
| LCDR2 | 417 | Kabat | FATNRYT |
| LCDR3 | 418 | Kabat | QQDYSSPWT |
| LCDR1 | 422 | IMGT | QSVSND |
| LCDR2 | 420 | IMGT | FAT |
| LCDR1 | 418 | IMGT | QQDYSSPWT |
| Trop2 | | | |
| Datopotamab | | | |
| HCDR1 | 423 | Combined | GYTFTTAGMQ |
| HCDR2 | 424 | Combined | WINTHSGVPKYAEDFKG |
| HCDR3 | 425 | Combined | SGFGSSYWYFDV |
| HCDR1 | 426 | Chothia | GYTFTTA |
| HCDR2 | 427 | Chothia | NTHSGV |
| HCDR3 | 425 | Chothia | SGFGSSYWYFDV |
| HCDR1 | 428 | Kabat | TAGMQ |
| HCDR2 | 424 | Kabat | WINTHSGVPKYAEDFKG |
| HCDR3 | 425 | Kabat | SGFGSSYWYFDV |
| HCDR1 | 438 | IMGT | GYTFTTAG |
| HCDR2 | 429 | IMGT | INTHSGVP |
| HCDR3 | 430 | IMGT | ARSGFGSSYWYFDV |
| Trop2 | | | |
| Datopotamab | | | |
| LCDR1 | 431 | Combined | KASQDVSTAVA |
| LCDR2 | 432 | Combined | SASYRYT |
| LCDR3 | 433 | Combined | QQHYITPLT |
| LCDR1 | 434 | Chothia | SQDVSTA |
| LCDR2 | 298 | Chothia | SAS |
| LCDR3 | 435 | Chothia | HYITPL |
| LCDR1 | 431 | Kabat | KASQDVSTAVA |
| LCDR2 | 432 | Kabat | SASYRYT |
| LCDR3 | 433 | Kabat | QQHYITPLT |
| LCDR1 | 436 | IMGT | QDVSTA |
| LCDR2 | 298 | IMGT | SAS |
| LCDR1 | 433 | IMGT | QQHYITPLT |

In some embodiments, the antibody or antigen-binding fragment of an ADC disclosed herein may comprise any set of heavy and light chain variable domains listed in the tables above or a set of six CDRs from any set of heavy and light chain variable domains listed in the tables above. In some embodiments, the antibody or antigen-binding fragment of an ADC disclosed herein may comprise amino acid sequences that are conservatively modified and/or homologous to the sequences listed in the tables above, so long as the ADC retains the ability to bind to its target cancer antigen (e.g., with a K_{D} of less than 1×10⁻⁸M) and retains one or more functional properties of the ADCs disclosed herein (e.g., ability to internalize, bind to an antigen target, e.g., an antigen expressed on a tumor or other cancer cell, etc.).

In some embodiments, the antibody or antigen-binding fragment of an ADC disclosed herein further comprises human heavy and light chain constant domains or fragments thereof. For instance, the antibody or antigen-binding fragment of the described ADCs may comprise a human IgG heavy chain constant domain (such as an IgGI) and a human kappa or lambda light chain constant domain. In some embodiments, the antibody or antigen-binding fragment of the described ADCs comprises a human immunoglobulin G subtype 1 (IgGI) heavy chain constant domain with a human Ig kappa light chain constant domain.

In some embodiments, the target cancer antigen for an ADC is PCAD.

In some embodiments, the anti-PCAD antibody or antigen-binding fragment thereof comprises three heavy chain CDRs and three light chain CDRs as follows: heavy chain CDR1 (HCDR1) consisting of SEQ ID NO:33, heavy chain CDR2 (HCDR2) consisting of SEQ ID NO:34, heavy chain CDR3 (HCDR3) consisting of SEQ ID NO:35; light chain CDR1 (LCDR1) consisting of SEQ ID NO:36, light chain CDR2 (LCDR2) consisting of SEQ ID NO:37, and light chain CDR3 (LCDR3) consisting of SEQ ID NO:38.

In some embodiments, the anti-PCAD antibody or antigen-binding fragment thereof comprises three heavy chain CDRs and three light chain CDRs as follows: heavy chain CDR1 (HCDR1) consisting of SEQ ID NO:304, heavy chain CDR2 (HCDR2) consisting of SEQ ID NO:305, heavy chain CDR3 (HCDR3) consisting of SEQ ID NO:306; light chain CDR1 (LCDR1) consisting of SEQ ID NO:312, light chain CDR2 (LCDR2) consisting of SEQ ID NO:313, and light chain CDR3 (LCDR3) consisting of SEQ ID NO:314.

In some embodiments, the anti-PCAD antibody or antigen-binding fragment thereof comprises three heavy chain CDRs and three light chain CDRs as follows: heavy chain CDR1 (HCDR1) consisting of SEQ ID NO:307, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316.

In some embodiments, the anti-PCAD antibody or antigen-binding fragment thereof comprises three heavy chain CDRs and three light chain CDRs as follows: heavy chain CDR1 (HCDR1) consisting of SEQ ID NO:309, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:317, light chain CDR2 (LCDR2) consisting of SEQ ID NO:313, and light chain CDR3 (LCDR3) consisting of SEQ ID NO:316.

In some embodiments, the anti-PCAD antibody or antigen-binding fragment thereof comprises three heavy chain CDRs and three light chain CDRs as follows: heavy chain CDR1 (HCDR1) consisting of SEQ ID NO:310, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316.

In some embodiments, the anti-PCAD antibody or antigen-binding fragment thereof comprises a heavy chain variable region comprising the amino acid sequence of SEQ ID NO:7, and a light chain variable region comprising the amino acid sequence of SEQ ID NO:8. In some embodiments, the anti-PCAD antibody or antigen-binding fragment thereof comprises the heavy chain variable region amino acid sequence of SEQ ID NO:7 and the light chain variable region amino acid sequence of SEQ ID NO:8, or sequences that are at least 95% identical to the disclosed sequences. In some embodiments, the anti-PCAD antibody or antigen-binding fragment thereof has a heavy chain variable region amino acid sequence that is at least 96%, at least 97%, at least 98%, or at least 99% identical to SEQ ID NO:7 and/or a light chain variable region amino acid sequence that is at least 96%, at least 97%, at least 98%, or at least 99% identical to SEQ ID NO:8.

In some embodiments, the anti-PCAD antibody or antigen-binding fragment thereof comprises a heavy chain variable region comprising the amino acid sequence of SEQ ID NO:303, and a light chain variable region comprising the amino acid sequence of SEQ ID NO:311. In some embodiments, the anti-PCAD antibody or antigen-binding fragment thereof comprises the heavy chain variable region amino acid sequence of SEQ ID NO:303 and the light chain variable region amino acid sequence of SEQ ID NO:311, or sequences that are at least 95% identical to the disclosed sequences. In some embodiments, the anti-PCAD antibody or antigen-binding fragment thereof has a heavy chain variable region amino acid sequence that is at least 96%, at least 97%, at least 98%, or at least 99% identical to SEQ ID NO:303 and/or a light chain variable region amino acid sequence that is at least 96%, at least 97%, at least 98%, or at least 99% identical to SEQ ID NO:311.

In some embodiments, the anti-PCAD antibody or antigen-binding fragment thereof is an internalizing antibody or internalizing antigen-binding fragment. In some embodiments, the anti-PCAD antibody comprises a human IgGI heavy chain constant domain or a modified IgGI heavy chain constant domain. In some embodiments, the IgGI heavy chain constant domain comprises a cysteine residue (C) at the amino acid positions corresponding to 152 and 375 in a wild-type (unmodified) IgGI heavy chain constant domain numbered according to EU numbering system.

In some embodiments, the anti-PCAD antibody comprises the heavy chain amino acid sequence of SEQ ID NO:63 or a sequence that is at least 95% identical to SEQ ID NO:63, and the light chain amino acid sequence of SEQ ID NO:64 or a sequence that is at least 95% identical to SEQ ID NO:64. In some embodiments, the anti-PCAD antibody comprises the heavy chain amino acid sequence of SEQ ID NO:63 and the light chain amino acid sequence of SEQ ID NO:64, or sequences that are at least 95% identical to the disclosed sequences. In some embodiments, the anti-PCAD antibody has a heavy chain amino acid sequence that is at least 96%, at least 97%, at least 98%, or at least 99% identical to SEQ ID NO:63 and a light chain amino acid sequence that is at least 96%, at least 97%, at least 98%, or at least 99% identical to SEQ ID NO:64. In some embodiments, the anti-PCAD antibody is NOV169N31Q (WO 2016/203432), or an antigen-binding fragment thereof.

In some embodiments, the anti-PCAD antibody comprises the heavy chain amino acid sequence of SEQ ID NO:248 or a sequence that is at least 95% identical to SEQ ID NO:248, and the light chain amino acid sequence of SEQ ID NO:250 or a sequence that is at least 95% identical to SEQ ID NO:250. In some embodiments, the anti-PCAD antibody comprises the heavy chain amino acid sequence of SEQ ID NO:248 and the light chain amino acid sequence of SEQ ID NO:250, or sequences that are at least 95% identical to the disclosed sequences. In some embodiments, the anti-PCAD antibody has a heavy chain amino acid sequence that is at least 96%, at least 97%, at least 98%, or at least 99% identical to SEQ ID NO:248 and a light chain amino acid sequence that is at least 96%, at least 97%, at least 98%, or at least 99% identical to SEQ ID NO:250.

In some embodiments, the anti-PCAD antibody or antigen-binding fragment thereof comprises the three heavy chain CDRs and three light chain CDRs of CQY679 or wherein the CDRs include no more than one, two, three, four, five, or six amino acid additions, deletions or substitutions of HCDR1 (SEQ ID NO:304), HCDR2 (SEQ ID NO:305), HCDR3 (SEQ ID NO:306); LCDR1 (SEQ ID NO:312), LCDR2 (SEQ ID NO:313), and LCDR3 (SEQ ID NO:314).

In some embodiments, the anti-PCAD antibody or antigen-binding fragment thereof comprises the three heavy chain CDRs and three light chain CDRs of CQY679 or wherein the CDRs include no more than one, two, three, four, five, or six amino acid additions, deletions or substitutions of HCDR1 (SEQ ID NO:307), HCDR2 (SEQ ID NO:308), HCDR3 (SEQ ID NO:306); LCDR1 (SEQ ID NO:315), LCDR2 (SEQ ID NO:25), and LCDR3 (SEQ ID NO:316).

In some embodiments, the anti-PCAD antibody or antigen-binding fragment thereof comprises the three heavy chain CDRs and three light chain CDRs of CQY679 or wherein the CDRs include no more than one, two, three, four, five, or six amino acid additions, deletions or substitutions of HCDR1 (SEQ ID NO:309), HCDR2 (SEQ ID NO:277), HCDR3 (SEQ ID NO:278); LCDR1 (SEQ ID NO:317), LCDR2 (SEQ ID NO:313), and LCDR3 (SEQ ID NO:316).

In some embodiments, the anti-PCAD antibody or antigen-binding fragment thereof comprises the three heavy chain CDRs and three light chain CDRs of CQY679 or wherein the CDRs include no more than one, two, three, four, five, or six amino acid additions, deletions or substitutions of HCDR1 (SEQ ID NO:310), HCDR2 (SEQ ID NO:308), HCDR3 (SEQ ID NO:306); LCDR1 (SEQ ID NO:315), LCDR2 (SEQ ID NO:25), and LCDR3 (SEQ ID NO:316).

In some embodiments, the anti-PCAD antibody or antigen-binding fragment thereof comprises the three heavy chain CDRs and three light chain CDRs of NOV169N31Q or wherein the CDRs include no more than one, two, three, four, five, or six amino acid additions, deletions or substitutions of HCDR1 (SEQ ID NO:33), HCDR2 (SEQ ID NO:34), HCDR3 (SEQ ID NO:35); LCDR1 (SEQ ID NO:36), LCDR2 (SEQ ID NO:37), and LCDR3 (SEQ ID NO:38).

In some embodiments, the target cancer antigen for an ADC is HER2.

In some embodiments, the anti-HER2 antibody or antigen-binding fragment thereof comprises three heavy chain CDRs and three light chain CDRs as follows: heavy chain CDR1 (HCDR1) consisting of SEQ ID NO:39, heavy chain CDR2 (HCDR2) consisting of SEQ ID NO:40, heavy chain CDR3 (HCDR3) consisting of SEQ ID NO:41; light chain CDR1 (LCDR1) consisting of SEQ ID NO:42, light chain CDR2 (LCDR2) consisting of SEQ ID NO:43, and light chain CDR3 (LCDR3) consisting of SEQ ID NO:44.

In some embodiments, the anti-HER2 antibody or antigen-binding fragment thereof comprises three heavy chain CDRs and three light chain CDRs as follows: heavy chain CDR1 (HCDR1) consisting of SEQ ID NO:289, heavy chain CDR2 (HCDR2) consisting of SEQ ID NO:290, heavy chain CDR3 (HCDR3) consisting of SEQ ID NO:291; light chain CDR1 (LCDR1) consisting of SEQ ID NO:297, light chain CDR2 (LCDR2) consisting of SEQ ID NO:298, and light chain CDR3 (LCDR3) consisting of SEQ ID NO:299.

In some embodiments, the anti-HER2 antibody or antigen-binding fragment thereof comprises three heavy chain CDRs and three light chain CDRs as follows: heavy chain CDR1 (HCDR1) consisting of SEQ ID NO:292, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44.

In some embodiments, the anti-HER2 antibody or antigen-binding fragment thereof comprises three heavy chain CDRs and three light chain CDRs as follows: heavy chain CDR1 (HCDR1) consisting of SEQ ID NO:293, heavy chain CDR2 (HCDR2) consisting of SEQ ID NO:294, heavy chain CDR3 (HCDR3) consisting of SEQ ID NO:295; light chain CDR1 (LCDR1) consisting of SEQ ID NO:302, light chain CDR2 (LCDR2) consisting of SEQ ID NO:298, and light chain CDR3 (LCDR3) consisting of SEQ ID NO:44.

In some embodiments, the anti-HER2 antibody or antigen-binding fragment thereof comprises three heavy chain CDRs and three light chain CDRs as follows: heavy chain CDR1 (HCDR1) consisting of SEQ ID NO:39, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44.

In some embodiments, the anti-HER2 antibody or antigen-binding fragment thereof comprises a heavy chain variable region comprising the amino acid sequence of SEQ ID NO:9, and a light chain variable region comprising the amino acid sequence of SEQ ID NO:10. In some embodiments, the anti-HER2 antibody or antigen-binding fragment thereof comprises the heavy chain variable region amino acid sequence of SEQ ID NO:9 and the light chain variable region amino acid sequence of SEQ ID NO:10, or sequences that are at least 95% identical to the disclosed sequences. In some embodiments, the anti-HER2 antibody or antigen-binding fragment thereof has a heavy chain variable region amino acid sequence that is at least 96%, at least 97%, at least 98%, or at least 99% identical to SEQ ID NO:9 and/or a light chain variable region amino acid sequence that is at least 96%, at least 97%, at least 98%, or at least 99% identical to SEQ ID NO:10.

In some embodiments, the anti-HER2 antibody or antigen-binding fragment thereof comprises a heavy chain variable region comprising the amino acid sequence of SEQ ID NO:9, and a light chain variable region comprising the amino acid sequence of SEQ ID NO:296. In some embodiments, the anti-HER2 antibody or antigen-binding fragment thereof comprises the heavy chain variable region amino acid sequence of SEQ ID NO:9 and the light chain variable region amino acid sequence of SEQ ID NO:296, or sequences that are at least 95% identical to the disclosed sequences. In some embodiments, the anti-HER2 antibody or antigen-binding fragment thereof has a heavy chain variable region amino acid sequence that is at least 96%, at least 97%, at least 98%, or at least 99% identical to SEQ ID NO:9 and/or a light chain variable region amino acid sequence that is at least 96%, at least 97%, at least 98%, or at least 99% identical to SEQ ID NO:296.

In some embodiments, the anti-HER2 antibody or antigen-binding fragment thereof is an internalizing antibody or internalizing antigen-binding fragment. In some embodiments, the anti-HER2 antibody comprises a human IgGI heavy chain constant domain or a modified IgGI heavy chain constant domain. In some embodiments, the IgGI heavy chain constant domain comprises a glutamine residue (Q) at the amino acid position corresponding to 297 in a wild-type (unmodified) IgGI heavy chain constant domain. In some embodiments, the IgGI heavy chain constant domain comprises a serine residue (S) at the amino acid position corresponding to 297 in a wild-type (unmodified) IgGI heavy chain constant domain. In some embodiments, the IgGI heavy chain constant domain comprises a cysteine residue (C) at the amino acid positions corresponding to 152 and 375 in a wild-type (unmodified) IgGI heavy chain constant domain numbered according to EU numbering system. In some embodiments, the anti-HER2 antibody comprises a human Ig kappa light chain constant domain or a modified Ig kappa light chain constant domain.

In some embodiments, the anti-HER2 antibody comprises the heavy chain amino acid sequence of SEQ ID NO:65 or a sequence that is at least 95% identical to SEQ ID NO:65, and the light chain amino acid sequence of SEQ ID NO:66 or a sequence that is at least 95% identical to SEQ ID NO:66. In some embodiments, the anti-HER2 antibody comprises the heavy chain amino acid sequence of SEQ ID NO:65 and the light chain amino acid sequence of SEQ ID NO:66, or sequences that are at least 95% identical to the disclosed sequences. In some embodiments, the anti-HER2 antibody has a heavy chain amino acid sequence that is at least 96%, at least 97%, at least 98%, or at least 99% identical to SEQ ID NO:65 and a light chain amino acid sequence that is at least 96%, at least 97%, at least 98%, or at least 99% identical to SEQ ID NO:66. In some embodiments, the anti-HER2 antibody is trastuzumab (U.S. Pat. Nos. 5,821,337 and 6,870,034; see also Molina et al. (2001) Cancer Res. 61(12):4744-9), or an antigen-binding fragment thereof.

In some embodiments, the anti-HER2 antibody comprises the heavy chain amino acid sequence of SEQ ID NO:245 or a sequence that is at least 95% identical to SEQ ID NO:245, and the light chain amino acid sequence of SEQ ID NO:66 or a sequence that is at least 95% identical to SEQ ID NO:66. In some embodiments, the anti-HER2 antibody comprises the heavy chain amino acid sequence of SEQ ID NO:245 and the light chain amino acid sequence of SEQ ID NO:66, or sequences that are at least 95% identical to the disclosed sequences. In some embodiments, the anti-HER2 antibody has a heavy chain amino acid sequence that is at least 96%, at least 97%, at least 98%, or at least 99% identical to SEQ ID NO:245 and a light chain amino acid sequence that is at least 96%, at least 97%, at least 98%, or at least 99% identical to SEQ ID NO:66.

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:39), HCDR2 (SEQ ID NO:40), HCDR3 (SEQ ID NO:41); LCDR1 (SEQ ID NO:42), LCDR2 (SEQ ID NO:43), and LCDR3 (SEQ ID NO:44).

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:289), HCDR2 (SEQ ID NO:290), HCDR3 (SEQ ID NO:291); LCDR1 (SEQ ID NO:297), LCDR2 (SEQ ID NO:298), and LCDR3 (SEQ ID NO:299).

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:292), HCDR2 (SEQ ID NO:40), HCDR3 (SEQ ID NO:291); LCDR1 (SEQ ID NO:300), LCDR2 (SEQ ID NO:301), and LCDR3 (SEQ ID NO:44).

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:293), HCDR2 (SEQ ID NO:294), HCDR3 (SEQ ID NO:295); LCDR1 (SEQ ID NO:302), LCDR2 (SEQ ID NO:298), and LCDR3 (SEQ ID NO:44).

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:39), HCDR2 (SEQ ID NO:40), HCDR3 (SEQ ID NO:291); LCDR1 (SEQ ID NO:300), LCDR2 (SEQ ID NO:301), and LCDR3 (SEQ ID NO:44).

In some embodiments, the target cancer antigen for an ADC is CD48.

In some embodiments, the anti-CD48 antibody or antigen-binding fragment thereof comprises three heavy chain CDRs and three light chain CDRs as follows: heavy chain CDR1 (HCDR1) consisting of SEQ ID NO:51, heavy chain CDR2 (HCDR2) consisting of SEQ ID NO:52, heavy chain CDR3 (HCDR3) consisting of SEQ ID NO:53; light chain CDR1 (LCDR1) consisting of SEQ ID NO:54, light chain CDR2 (LCDR2) consisting of SEQ ID NO:55, and light chain CDR3 (LCDR3) consisting of SEQ ID NO:56.

In some embodiments, the anti-CD48 antibody or antigen-binding fragment thereof comprises three heavy chain CDRs and three light chain CDRs as follows: heavy chain CDR1 (HCDR1) consisting of SEQ ID NO:271, heavy chain CDR2 (HCDR2) consisting of SEQ ID NO:272, heavy chain CDR3 (HCDR3) consisting of SEQ ID NO:273; light chain CDR1 (LCDR1) consisting of SEQ ID NO:281, light chain CDR2 (LCDR2) consisting of SEQ ID NO:282, and light chain CDR3 (LCDR3) consisting of SEQ ID NO:283.

In some embodiments, the anti-CD48 antibody or antigen-binding fragment thereof comprises three heavy chain CDRs and three light chain CDRs as follows: heavy chain CDR1 (HCDR1) consisting of SEQ ID NO:274, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:285, and light chain CDR3 (LCDR3) consisting of SEQ ID NO:286.

In some embodiments, the anti-CD48 antibody or antigen-binding fragment thereof comprises three heavy chain CDRs and three light chain CDRs as follows: heavy chain CDR1 (HCDR1) consisting of SEQ ID NO:276, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:287, light chain CDR2 (LCDR2) consisting of SEQ ID NO:282, and light chain CDR3 (LCDR3) consisting of SEQ ID NO:286.

In some embodiments, the anti-CD48 antibody or antigen-binding fragment thereof comprises three heavy chain CDRs and three light chain CDRs as follows: heavy chain CDR1 (HCDR1) consisting of SEQ ID NO:279, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:288, and light chain CDR3 (LCDR3) consisting of SEQ ID NO:286.

In some embodiments, the anti-CD48 antibody or antigen-binding fragment thereof comprises a heavy chain variable region comprising the amino acid sequence of SEQ ID NO:13, and a light chain variable region comprising the amino acid sequence of SEQ ID NO:14. In some embodiments, the anti-CD48 antibody or antigen-binding fragment thereof comprises the heavy chain variable region amino acid sequence of SEQ ID NO:13 and the light chain variable region amino acid sequence of SEQ ID NO:14, or sequences that are at least 95% identical to the disclosed sequences. In some embodiments, the anti-CD48 antibody or antigen-binding fragment thereof has a heavy chain variable region amino acid sequence that is at least 96%, at least 97%, at least 98%, or at least 99% identical to SEQ ID NO:13 and/or a light chain variable region amino acid sequence that is at least 96%, at least 97%, at least 98%, or at least 99% identical to SEQ ID NO:14.

In some embodiments, the anti-CD48 antibody or antigen-binding fragment thereof comprises a heavy chain variable region comprising the amino acid sequence of SEQ ID NO:270, and a light chain variable region comprising the amino acid sequence of SEQ ID NO:280. In some embodiments, the anti-CD48 antibody or antigen-binding fragment thereof comprises the heavy chain variable region amino acid sequence of SEQ ID NO:270 and the light chain variable region amino acid sequence of SEQ ID NO:280, or sequences that are at least 95% identical to the disclosed sequences. In some embodiments, the anti-CD48 antibody or antigen-binding fragment thereof has a heavy chain variable region amino acid sequence that is at least 96%, at least 97%, at least 98%, or at least 99% identical to SEQ ID NO:270 and/or a light chain variable region amino acid sequence that is at least 96%, at least 97%, at least 98%, or at least 99% identical to SEQ ID NO:280.

In some embodiments, the anti-CD48 antibody or antigen-binding fragment thereof is an internalizing antibody or internalizing antigen-binding fragment. In some embodiments, the anti-CD48 antibody comprises a human IgGI heavy chain constant domain or a modified IgGI heavy chain constant domain. In some embodiments, the IgGI heavy chain constant domain comprises a cysteine residue (C) at the amino acid positions corresponding to 152 and 375 in a wild-type (unmodified) IgGI heavy chain constant domain numbered according to EU numbering system.

In some embodiments, the anti-CD48 antibody comprises the heavy chain amino acid sequence of SEQ ID NO:69 or a sequence that is at least 95% identical to SEQ ID NO:69, and the light chain amino acid sequence of SEQ ID NO:70 or a sequence that is at least 95% identical to SEQ ID NO:70. In some embodiments, the anti-CD48 antibody comprises the heavy chain amino acid sequence of SEQ ID NO:69 and the light chain amino acid sequence of SEQ ID NO:70, or sequences that are at least 95% identical to the disclosed sequences. In some embodiments, the anti-CD48 antibody has a heavy chain amino acid sequence that is at least 96%, at least 97%, at least 98%, or at least 99% identical to SEQ ID NO:69 and a light chain amino acid sequence that is at least 96%, at least 97%, at least 98%, or at least 99% identical to SEQ ID NO:70. In some embodiments, the anti-CD48 antibody is SGN-48A, or an antigen-binding fragment thereof.

In some embodiments, the anti-CD48 antibody comprises the heavy chain amino acid sequence of SEQ ID NO:240 or a sequence that is at least 95% identical to SEQ ID NO:240, and the light chain amino acid sequence of SEQ ID NO:243 or a sequence that is at least 95% identical to SEQ ID NO:243. In some embodiments, the anti-CD48 antibody comprises the heavy chain amino acid sequence of SEQ ID NO:240 and the light chain amino acid sequence of SEQ ID NO:243, or sequences that are at least 95% identical to the disclosed sequences. In some embodiments, the anti-CD48 antibody has a heavy chain amino acid sequence that is at least 96%, at least 97%, at least 98%, or at least 99% identical to SEQ ID NO:240 and a light chain amino acid sequence that is at least 96%, at least 97%, at least 98%, or at least 99% identical to SEQ ID NO:243.

In some embodiments, the anti-CD48 antibody comprises the heavy chain amino acid sequence of SEQ ID NO:242 or a sequence that is at least 95% identical to SEQ ID NO:242, and the light chain amino acid sequence of SEQ ID NO:243 or a sequence that is at least 95% identical to SEQ ID NO:243. In some embodiments, the anti-CD48 antibody comprises the heavy chain amino acid sequence of SEQ ID NO:242 and the light chain amino acid sequence of SEQ ID NO:243, or sequences that are at least 95% identical to the disclosed sequences. In some embodiments, the anti-CD48 antibody has a heavy chain amino acid sequence that is at least 96%, at least 97%, at least 98%, or at least 99% identical to SEQ ID NO:242 and a light chain amino acid sequence that is at least 96%, at least 97%, at least 98%, or at least 99% identical to SEQ ID NO:243.

In some embodiments, the anti-CD48 antibody or antigen-binding fragment thereof comprises the three heavy chain CDRs and three light chain CDRs of SGN-48A or wherein the CDRs include no more than one, two, three, four, five, or six amino acid additions, deletions or substitutions of HCDR1 (SEQ ID NO:51), HCDR2 (SEQ ID NO:52), HCDR3 (SEQ ID NO:53); LCDR1 (SEQ ID NO:54), LCDR2 (SEQ ID NO:55), and LCDR3 (SEQ ID NO:56).

In some embodiments, the anti-CD48 antibody or antigen-binding fragment thereof comprises the three heavy chain CDRs and three light chain CDRs of NY920 or wherein the CDRs include no more than one, two, three, four, five, or six amino acid additions, deletions or substitutions of HCDR1 (SEQ ID NO:271), HCDR2 (SEQ ID NO:272), HCDR3 (SEQ ID NO:273); LCDR1 (SEQ ID NO:281), LCDR2 (SEQ ID NO:282), and LCDR3 (SEQ ID NO:283).

In some embodiments, the anti-CD48 antibody or antigen-binding fragment thereof comprises the three heavy chain CDRs and three light chain CDRs of NY920 or wherein the CDRs include no more than one, two, three, four, five, or six amino acid additions, deletions or substitutions of HCDR1 (SEQ ID NO:274), HCDR2 (SEQ ID NO:275), HCDR3 (SEQ ID NO:273); LCDR1 (SEQ ID NO:284), LCDR2 (SEQ ID NO:285), and LCDR3 (SEQ ID NO:286).

In some embodiments, the anti-CD48 antibody or antigen-binding fragment thereof comprises the three heavy chain CDRs and three light chain CDRs of NY920 or wherein the CDRs include no more than one, two, three, four, five, or six amino acid additions, deletions or substitutions of HCDR1 (SEQ ID NO:276), HCDR2 (SEQ ID NO:277), HCDR3 (SEQ ID NO:278); LCDR1 (SEQ ID NO:287), LCDR2 (SEQ ID NO:282), and LCDR3 (SEQ ID NO:286).

In some embodiments, the anti-CD48 antibody or antigen-binding fragment thereof comprises the three heavy chain CDRs and three light chain CDRs of NY920 or wherein the CDRs include no more than one, two, three, four, five, or six amino acid additions, deletions or substitutions of HCDR1 (SEQ ID NO:279), HCDR2 (SEQ ID NO:275), HCDR3 (SEQ ID NO:273); LCDR1 (SEQ ID NO:284), LCDR2 (SEQ ID NO:288), and LCDR3 (SEQ ID NO:286).

In some embodiments, the target antigen for an ADC is CD74.

In some embodiments, the anti-CD74 antibody comprises the heavy chain amino acid sequence of SEQ ID NO:118 or a sequence that is at least 95% identical to SEQ ID NO:118, and the light chain amino acid sequence of SEQ ID NO:119 or a sequence that is at least 95% identical to SEQ ID NO:119. In some embodiments, the anti-CD74 antibody comprises the heavy chain amino acid sequence of SEQ ID NO:118 and the light chain amino acid sequence of SEQ ID NO:119, or sequences that are at least 95% identical to the disclosed sequences. In some embodiments, the anti-CD74 antibody has a heavy chain amino acid sequence that is at least 96%, at least 97%, at least 98%, or at least 99% identical to SEQ ID NO:118 and a light chain amino acid sequence that is at least 96%, at least 97%, at least 98%, or at least 99% identical to SEQ ID NO:119. In some embodiments, the anti-CD74 antibody is milatuzumab, or an antigen-binding fragment thereof.

In some embodiments, the anti-CD74 antibody comprises the heavy chain amino acid sequence of SEQ ID NO:118 or a sequence that is at least 95% identical to SEQ ID NO:118, and the light chain amino acid sequence of SEQ ID NO:237 or a sequence that is at least 95% identical to SEQ ID NO:237. In some embodiments, the anti-CD74 antibody comprises the heavy chain amino acid sequence of SEQ ID NO:118 and the light chain amino acid sequence of SEQ ID NO:237, or sequences that are at least 95% identical to the disclosed sequences. In some embodiments, the anti-CD74 antibody has a heavy chain amino acid sequence that is at least 96%, at least 97%, at least 98%, or at least 99% identical to SEQ ID NO:118 and a light chain amino acid sequence that is at least 96%, at least 97%, at least 98%, or at least 99% identical to SEQ ID NO:234.

In some embodiments, the anti-CD74 antibody comprises the heavy chain amino acid sequence of SEQ ID NO:236 or a sequence that is at least 95% identical to SEQ ID NO:236, and the light chain amino acid sequence of SEQ ID NO:237 or a sequence that is at least 95% identical to SEQ ID NO:237. In some embodiments, the anti-CD74 antibody comprises the heavy chain amino acid sequence of SEQ ID NO:236 and the light chain amino acid sequence of SEQ ID NO:237, or sequences that are at least 95% identical to the disclosed sequences. In some embodiments, the anti-CD74 antibody has a heavy chain amino acid sequence that is at least 96%, at least 97%, at least 98%, or at least 99% identical to SEQ ID NO:236 and a light chain amino acid sequence that is at least 96%, at least 97%, at least 98%, or at least 99% identical to SEQ ID NO:237.

In some embodiments, the anti-CD74 antibody comprises the heavy chain amino acid sequence of SEQ ID NO:118 or a sequence that is at least 95% identical to SEQ ID NO:118, and the light chain amino acid sequence of SEQ ID NO:239 or a sequence that is at least 95% identical to SEQ ID NO:239. In some embodiments, the anti-CD74 antibody comprises the heavy chain amino acid sequence of SEQ ID NO:118 and the light chain amino acid sequence of SEQ ID NO:239, or sequences that are at least 95% identical to the disclosed sequences. In some embodiments, the anti-CD74 antibody has a heavy chain amino acid sequence that is at least 96%, at least 97%, at least 98%, or at least 99% identical to SEQ ID NO:118 and a light chain amino acid sequence that is at least 96%, at least 97%, at least 98%, or at least 99% identical to SEQ ID NO:239.

In some embodiments, the anti-CD74 antibody or antigen-binding fragment thereof comprises a heavy chain variable region comprising the amino acid sequence of SEQ ID NO:153, and a light chain variable region comprising the amino acid sequence of SEQ ID NO:262. In some embodiments, the anti-CD74 antibody or antigen-binding fragment thereof comprises the heavy chain variable region amino acid sequence of SEQ ID NO:153 and the light chain variable region amino acid sequence of SEQ ID NO:262, or sequences that are at least 95% identical to the disclosed sequences. In some embodiments, the anti-CD74 antibody or antigen-binding fragment thereof has a heavy chain variable region amino acid sequence that is at least 96%, at least 97%, at least 98%, or at least 99% identical to SEQ ID NO:153 and/or a light chain variable region amino acid sequence that is at least 96%, at least 97%, at least 98%, or at least 99% identical to SEQ ID NO:262.

In some embodiments, the anti-CD74 antibody or antigen-binding fragment thereof comprises a heavy chain variable region comprising the amino acid sequence of SEQ ID NO:153, and a light chain variable region comprising the amino acid sequence of SEQ ID NO:267. In some embodiments, the anti-CD74 antibody or antigen-binding fragment thereof comprises the heavy chain variable region amino acid sequence of SEQ ID NO:153 and the light chain variable region amino acid sequence of SEQ ID NO:267, or sequences that are at least 95% identical to the disclosed sequences. In some embodiments, the anti-CD74 antibody or antigen-binding fragment thereof has a heavy chain variable region amino acid sequence that is at least 96%, at least 97%, at least 98%, or at least 99% identical to SEQ ID NO:153 and/or a light chain variable region amino acid sequence that is at least 96%, at least 97%, at least 98%, or at least 99% identical to SEQ ID NO:267.

In some embodiments, the anti-CD74 antibody or antigen-binding fragment thereof comprises three heavy chain CDRs and three light chain CDRs as follows: heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:263, light chain CDR2 (LCDR2) consisting of SEQ ID NO:264, and light chain CDR3 (LCDR3) consisting of SEQ ID NO:265.

In some embodiments, the anti-CD74 antibody or antigen-binding fragment thereof comprises three heavy chain CDRs and three light chain CDRs as follows: heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:215, light chain CDR2 (LCDR2) consisting of SEQ ID NO:264, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:268, light chain CDR2 (LCDR2) consisting of SEQ ID NO:264, and light chain CDR3 (LCDR3) consisting of SEQ ID NO:265.

In some embodiments, the anti-CD74 antibody or antigen-binding fragment thereof comprises three heavy chain CDRs and three light chain CDRs as follows: heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:269, light chain CDR2 (LCDR2) consisting of SEQ ID NO:264, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174.

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:256), HCDR2 (SEQ ID NO:257), HCDR3 (SEQ ID NO:171); LCDR1 (SEQ ID NO:263), LCDR2 (SEQ ID NO:264), and LCDR3 (SEQ ID NO:265).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:258), HCDR2 (SEQ ID NO:170), HCDR3 (SEQ ID NO:171); LCDR1 (SEQ ID NO:266), LCDR2 (SEQ ID NO:173), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:259), HCDR2 (SEQ ID NO:260), HCDR3 (SEQ ID NO:261); LCDR1 (SEQ ID NO:267), LCDR2 (SEQ ID NO:264), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:169), HCDR2 (SEQ ID NO:170), HCDR3 (SEQ ID NO:171); LCDR1 (SEQ ID NO:266), LCDR2 (SEQ ID NO:173), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:256), HCDR2 (SEQ ID NO:257), HCDR3 (SEQ ID NO:171); LCDR1 (SEQ ID NO:268), LCDR2 (SEQ ID NO:264), and LCDR3 (SEQ ID NO:265).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:258), HCDR2 (SEQ ID NO:170), HCDR3 (SEQ ID NO:171); LCDR1 (SEQ ID NO:172), LCDR2 (SEQ ID NO:173), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:259), HCDR2 (SEQ ID NO:260), HCDR3 (SEQ ID NO:261); LCDR1 (SEQ ID NO:269), LCDR2 (SEQ ID NO:264), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:169), HCDR2 (SEQ ID NO:170), HCDR3 (SEQ ID NO:171); LCDR1 (SEQ ID NO:172), LCDR2 (SEQ ID NO:173), and LCDR3 (SEQ ID NO:174).

In some embodiments, the target antigen for an ADC is EphA2.

In some embodiments, the anti- EphA2 antibody or antigen-binding fragment thereof comprises a heavy chain variable region comprising the amino acid sequence of SEQ ID NO:318, and a light chain variable region comprising the amino acid sequence of SEQ ID NO:329. In some embodiments, the anti-EphA2 antibody or antigen-binding fragment thereof comprises the heavy chain variable region amino acid sequence of SEQ ID NO:318 and the light chain variable region amino acid sequence of SEQ ID NO:329, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO:318 and/or a light chain variable region amino acid sequence that is at least 96%, at least 97%, at least 98%, or at least 99% identical to SEQ ID NO:329.

In some embodiments, the anti-EphA2 antibody comprises the heavy chain amino acid sequence of SEQ ID NO:252 or a sequence that is at least 95% identical to SEQ ID NO:252, and the light chain amino acid sequence of SEQ ID NO:254 or a sequence that is at least 95% identical to SEQ ID NO:254. In some embodiments, the anti-EphA2 antibody comprises the heavy chain amino acid sequence of SEQ ID NO:252 and the light chain amino acid sequence of SEQ ID NO:254, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO:252 and a light chain amino acid sequence that is at least 96%, at least 97%, at least 98%, or at least 99% identical to SEQ ID NO:254.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:319, heavy chain CDR2 (HCDR2) consisting of SEQ ID NO:320, heavy chain CDR3 (HCDR3) consisting of SEQ ID NO:321; light chain CDR1 (LCDR1) consisting of SEQ ID NO:330, light chain CDR2 (LCDR2) consisting of SEQ ID NO:331, and light chain CDR3 (LCDR3) consisting of SEQ ID NO:332.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:322, heavy chain CDR2 (HCDR2) consisting of SEQ ID NO:323, heavy chain CDR3 (HCDR3) consisting of SEQ ID NO:324; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:325, heavy chain CDR2 (HCDR2) consisting of SEQ ID NO:326, heavy chain CDR3 (HCDR3) consisting of SEQ ID NO:327; light chain CDR1 (LCDR1) consisting of SEQ ID NO:336, light chain CDR2 (LCDR2) consisting of SEQ ID NO:331, and light chain CDR3 (LCDR3) consisting of SEQ ID NO:335.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:328, heavy chain CDR2 (HCDR2) consisting of SEQ ID NO:323, heavy chain CDR3 (HCDR3) consisting of SEQ ID NO:321; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335.

In some embodiments, the anti-EphA2 antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:319), HCDR2 (SEQ ID NO:320), HCDR3 (SEQ ID NO:321); LCDR1 (SEQ ID NO:330), LCDR2 (SEQ ID NO:331), and LCDR3 (SEQ ID NO:332).

In some embodiments, the anti-EphA2 antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:322), HCDR2 (SEQ ID NO:323), HCDR3 (SEQ ID NO:324); LCDR1 (SEQ ID NO:333), LCDR2 (SEQ ID NO:334), and LCDR3 (SEQ ID NO:335).

In some embodiments, the anti-EphA2 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:325), HCDR2 (SEQ ID NO:326), HCDR3 (SEQ ID NO:327); LCDR1 (SEQ ID NO:336), LCDR2 (SEQ ID NO:331), and LCDR3 (SEQ ID NO:335).

In some embodiments, the anti-EphA2 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:328), HCDR2 (SEQ ID NO:323), HCDR3 (SEQ ID NO:321); LCDR1 (SEQ ID NO:333), LCDR2 (SEQ ID NO:334), and LCDR3 (SEQ ID NO:335).

In some embodiments, the target antigen for an ADC is MET.

In some embodiments, the anti-Met antibody or the antigen-binding fragment thereof comprises at least two, three, four or five CDR sequences selected from the group consisting of HCDR1 SEQ ID NO:349 or SEQ ID NO:355, HCDR2 SEQ ID NO:350 or SEQ ID NO:356, HCDR3 SEQ ID NO:351 or SEQ ID NO:357, LCDR1 SEQ ID NO:352 or SEQ ID NO:358, LCDR2 SEQ ID NO:353 or SEQ ID NO:359, and LCDR3 SEQ ID NO:354 or SEQ ID NO:360.

In some embodiments, the anti-Met antibody or the antigen-binding fragment thereof comprises at least two, three, four or five CDR sequences selected from the group consisting of HCDR1 SEQ ID NO: 349 or SEQ ID NO:355 or SEQ ID NO:361, HCDR2 SEQ ID NO: 350 or SEQ ID NO:356 or SEQ ID NO:362, HCDR3 SEQ ID NO: 351 or SEQ ID NO:357 or SEQ ID NO:363, LCDR1 SEQ ID NO:352 or SEQ ID NO:358 or SEQ ID NO:364, LCDR2 SEQ ID NO: 353 or SEQ ID NO:359 or SEQ ID NO:365, and LCDR3 SEQ ID NO: 354 or SEQ ID NO:360 or SEQ ID NO:366.

In some embodiments, the anti-Met antibody or antigen-binding fragment thereof comprises three heavy chain CDRs and three light chain CDRs as follows: heavy chain CDR1 (HCDR1) consisting of SEQ ID NO:349, heavy chain CDR2 (HCDR2) consisting of SEQ ID NO:350, heavy chain CDR3 (HCDR3) consisting of SEQ ID NO:351; light chain CDR1 (LCDR1) consisting of SEQ ID NO:352, light chain CDR2 (LCDR2) consisting of SEQ ID NO:353, and light chain CDR3 (LCDR3) consisting of SEQ ID NO:354.

In some embodiments, the anti-Met antibody or antigen-binding fragment thereof comprises three heavy chain CDRs and three light chain CDRs as follows: heavy chain CDR1 (HCDR1) consisting of SEQ ID NO:355, heavy chain CDR2 (HCDR2) consisting of SEQ ID NO:356, heavy chain CDR3 (HCDR3) consisting of SEQ ID NO:357; light chain CDR1 (LCDR1) consisting of SEQ ID NO:358, light chain CDR2 (LCDR2) consisting of SEQ ID NO:359, and light chain CDR3 (LCDR3) consisting of SEQ ID NO:360.

In some embodiments, the anti-Met antibody or antigen-binding fragment thereof comprises three heavy chain CDRs and three light chain CDRs as follows: heavy chain CDR1 (HCDR1) consisting of SEQ ID NO:361, heavy chain CDR2 (HCDR2) consisting of SEQ ID NO: 362, heavy chain CDR3 (HCDR3) consisting of SEQ ID NO:363; light chain CDR1 (LCDR1) consisting of SEQ ID NO:364, light chain CDR2 (LCDR2) consisting of SEQ ID NO:365, and light chain CDR3 (LCDR3) consisting of SEQ ID NO:366.

In some embodiments, the anti-Met antibody or antigen-binding fragment thereof comprises the heavy chain variable region amino acid sequence of SEQ ID NO:339 and the light chain variable region amino acid sequence of SEQ ID NO:340. In some embodiments, the anti-Met antibody or antigen-binding fragment thereof comprises the heavy chain variable region amino acid sequence of SEQ ID NO:339 and the light chain variable region amino acid sequence of SEQ ID NO:340, or sequences that are at least 95% identical to the disclosed sequences. In some embodiments, the anti-Met antibody or antigen-binding fragment thereof has a heavy chain variable region amino acid sequence that is at least 96%, at least 97%, at least 98%, or at least 99% identical to SEQ ID NO:339 and/or a light chain variable region amino acid sequence that is at least 96%, at least 97%, at least 98%, or at least 99% identical to SEQ ID NO:340.

In some embodiments, the anti-Met antibody or antigen-binding fragment thereof comprises the heavy chain variable region amino acid sequence of SEQ ID NO:341 and the light chain variable region amino acid sequence of SEQ ID NO:342. In some embodiments, the anti-Met antibody or antigen-binding fragment thereof comprises the heavy chain variable region amino acid sequence of SEQ ID NO:341 and the light chain variable region amino acid sequence of SEQ ID NO:342, or sequences that are at least 95% identical to the disclosed sequences. In some embodiments, the anti-Met antibody or antigen-binding fragment thereof has a heavy chain variable region amino acid sequence that is at least 96%, at least 97%, at least 98%, or at least 99% identical to SEQ ID NO:341 and/or a light chain variable region amino acid sequence that is at least 96%, at least 97%, at least 98%, or at least 99% identical to SEQ ID NO:342.

In some embodiments, the anti-Met antibody or antigen-binding fragment thereof comprises the heavy chain variable region amino acid sequence of SEQ ID NO:343 and the light chain variable region amino acid sequence of SEQ ID NO:344. In some embodiments, the anti-Met antibody or antigen-binding fragment thereof comprises the heavy chain variable region amino acid sequence of SEQ ID NO:343 and the light chain variable region amino acid sequence of SEQ ID NO:344, or sequences that are at least 95% identical to the disclosed sequences. In some embodiments, the anti-Met antibody or antigen-binding fragment thereof has a heavy chain variable region amino acid sequence that is at least 96%, at least 97%, at least 98%, or at least 99% identical to SEQ ID NO: 343 and/or a light chain variable region amino acid sequence that is at least 96%, at least 97%, at least 98%, or at least 99% identical to SEQ ID NO: 344.

In some embodiments, the anti-Met antibody comprises the heavy chain amino acid sequence of SEQ ID NO:367 or a sequence that is at least 95% identical to SEQ ID NO: 367, and the light chain amino acid sequence of SEQ ID NO:368 or a sequence that is at least 95% identical to SEQ ID NO:368. In some embodiments, the anti-Met antibody comprises the heavy chain amino acid sequence of SEQ ID NO:367 and the light chain amino acid sequence of SEQ ID NO:368, or sequences that are at least 95% identical to the disclosed sequences. In some embodiments, the anti-Met antibody has a heavy chain amino acid sequence that is at least 96%, at least 97%, at least 98%, or at least 99% identical to SEQ ID NO:367 and a light chain amino acid sequence that is at least 96%, at least 97%, at least 98%, or at least 99% identical to SEQ ID NO:368.

In some embodiments, the anti-Met antibody comprises the heavy chain amino acid sequence of SEQ ID NO:369 or a sequence that is at least 95% identical to SEQ ID NO:369, and the light chain amino acid sequence of SEQ ID NO:370 or a sequence that is at least 95% identical to SEQ ID NO:370. In some embodiments, the anti-Met antibody comprises the heavy chain amino acid sequence of SEQ ID NO:369 and the light chain amino acid sequence of SEQ ID NO:370 or sequences that are at least 95% identical to the disclosed sequences. In some embodiments, the anti-Met antibody has a heavy chain amino acid sequence that is at least 96%, at least 97%, at least 98%, or at least 99% identical to SEQ ID NO:369 and a light chain amino acid sequence that is at least 96%, at least 97%, at least 98%, or at least 99% identical to SEQ ID NO:370.

In some embodiments, the anti-Met antibody comprises the heavy chain amino acid sequence of SEQ ID NO:371 or a sequence that is at least 95% identical to SEQ ID NO:371, and the light chain amino acid sequence of SEQ ID NO:372 or a sequence that is at least 95% identical to SEQ ID NO:372. In some embodiments, the anti-Met antibody comprises the heavy chain amino acid sequence of SEQ ID NO:371 and the light chain amino acid sequence of SEQ ID NO:372, or sequences that are at least 95% identical to the disclosed sequences. In some embodiments, the anti-Met antibody has a heavy chain amino acid sequence that is at least 96%, at least 97%, at least 98%, or at least 99% identical to SEQ ID NO:371 and a light chain amino acid sequence that is at least 96%, at least 97%, at least 98%, or at least 99% identical to SEQ ID NO:372.

In some embodiments, the anti-Met antibody comprises the heavy chain amino acid sequence of SEQ ID NO:373 or a sequence that is at least 95% identical to SEQ ID NO:373, and the light chain amino acid sequence of SEQ ID NO:374 or a sequence that is at least 95% identical to SEQ ID NO:374. In some embodiments, the anti-Met antibody comprises the heavy chain amino acid sequence of SEQ ID NO:373 and the light chain amino acid sequence of SEQ ID NO:374, or sequences that are at least 95% identical to the disclosed sequences. In some embodiments, the anti-Met antibody has a heavy chain amino acid sequence that is at least 96%, at least 97%, at least 98%, or at least 99% identical to SEQ ID NO:373 and a light chain amino acid sequence that is at least 96%, at least 97%, at least 98%, or at least 99% identical to SEQ ID NO:374.

In some embodiments, the anti-Met antibody comprises the heavy chain amino acid sequence of SEQ ID NO:375 or a sequence that is at least 95% identical to SEQ ID NO:375, and the light chain amino acid sequence of SEQ ID NO:370 or a sequence that is at least 95% identical to SEQ ID NO:370. In some embodiments, the anti-Met antibody comprises the heavy chain amino acid sequence of SEQ ID NO:375 and the light chain amino acid sequence of SEQ ID NO:370, or sequences that are at least 95% identical to the disclosed sequences. In some embodiments, the anti-Met antibody has a heavy chain amino acid sequence that is at least 96%, at least 97%, at least 98%, or at least 99% identical to SEQ ID NO:375 and a light chain amino acid sequence that is at least 96%, at least 97%, at least 98%, or at least 99% identical to SEQ ID NO:370.

In some embodiments, the anti-Met antibody comprises the heavy chain amino acid sequence of SEQ ID NO:376 or a sequence that is at least 95% identical to SEQ ID NO:376, and the light chain amino acid sequence of SEQ ID NO:372 or a sequence that is at least 95% identical to SEQ ID NO:372. In some embodiments, the anti-Met antibody comprises the heavy chain amino acid sequence of SEQ ID NO:376 and the light chain amino acid sequence of SEQ ID NO:372, or sequences that are at least 95% identical to the disclosed sequences. In some embodiments, the anti-Met antibody has a heavy chain amino acid sequence that is at least 96%, at least 97%, at least 98%, or at least 99% identical to SEQ ID NO:376 and a light chain amino acid sequence that is at least 96%, at least 97%, at least 98%, or at least 99% identical to SEQ ID NO:372.

Residues in two or more polypeptides are said to “correspond” if the residues occupy an analogous position in the polypeptide structures. Analogous positions in two or more polypeptides can be determined by aligning the polypeptide sequences based on amino acid sequence or structural similarities. Those skilled in the art understand that it may be necessary to introduce gaps in either sequence to produce a satisfactory alignment.

In some embodiments, amino acid substitutions are of single residues. Insertions usually will be on the order of from about 1 to about 20 amino acid residues, although considerably larger insertions may be tolerated as long as biological function is retained (e.g., binding to a target antigen). Deletions usually range from about 1 to about 20 amino acid residues, although in some cases deletions may be much larger. Substitutions, deletions, insertions, or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances. Conservative substitutions can be made in accordance with the following chart depicted as Table E.

TABLE E

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

In some embodiments where variant antibody sequences are used in an ADC, the variants typically exhibit the same qualitative biological activity and will elicit the same immune response, although variants may also be selected to modify the characteristics of the antigen binding proteins as needed. Alternatively, the variant may be designed such that the biological activity of the antigen binding protein is altered. For example, glycosylation sites may be altered or removed.

Various antibodies may be used with the ADCs used herein to target cancer cells. As shown below, the linker-payloads in the ADCs disclosed herein are surprisingly effective with different tumor antigen-targeting antibodies. Suitable antigens expressed on cancer cells but not healthy cells, or expressed on cancer cells at a higher level than on healthy cells, are known in the art, as are antibodies directed against them. Further antibodies against those antigen targets may be prepared by those of skill in the art. These antibodies may be used with the linkers and antineoplastic payloads, such as BH3 mimetics, topoisomerase 1 inhibitors, or anti-mitotic drugs, disclosed herein. In some embodiments, the antibody or antigen-binding fragment targets PCAD, and in some embodiments the PCAD-targeting antibody or antigen-binding fragment is NOV169N31Q. In some embodiments, the antibody or antigen-binding fragment targets PCAD, and in some embodiments the PCAD-targeting antibody or antigen-binding fragment is CQY679. In some embodiments, the antibody or antigen-binding fragment targets EPhA2, and in some embodiments the EphA2-targeting antibody or antigen-binding fragment is EphA2 1C₁. In some embodiments, the antibody or antigen-binding fragment targets CD74, and in some embodiments the CD74-targeting antibody or antigen-binding fragment is milatuzumab. In some embodiments, the antibody or antigen-binding fragment targets CD74, and in some embodiments the CD74-targeting antibody or antigen-binding fragment is VHmil x VK1aNQ. In some embodiments, the antibody or antigen-binding fragment targets CD48, and in some embodiments the CD48-targeting antibody or antigen-binding fragment is SGN-CD48A (MEM/MEM102). In some embodiments, the antibody or antigen-binding fragment targets CD48, and in some embodiments the CD48-targeting antibody or antigen-binding fragment is CD48 NY920. In some embodiments, the antibody or antigen-binding fragment targets HER2, and in some embodiments the HER2-targeting antibody or antigen-binding fragment is trastuzumab, and in some embodiments the HER2-targeting antibody or antigen-binding fragment is disitamab. In some embodiments, the antibody or antigen-binding fragment targets TROP2, and in some embodiments the TROP2-targeting antibody or antigen-binding fragment is datopotamab. In some embodiments, the antibody or antigen-binding fragment targets B7-H3, and in some embodiments the B7-H3-targeting antibody or antigen-binding fragment is ABBV-155, and in some embodiments the B7-H3-targeting antibody or antigen-binding fragment is DS-5573a. In some embodiments, the antibody or antigen-binding fragment targets 5T4. In some embodiments, the antibody or antigen-binding fragment targets MET, and in some embodiments the MET-targeting antibody or antigen-binding fragment is 9006, and in some embodiments the MET-targeting antibody or antigen-binding fragment is 9338, and in some embodiments the MET-targeting antibody or antigen-binding fragment is 8902.

In some embodiments, while the disclosed linkers and Bcl-xL inhibitor payloads are surprisingly effective with several different tumor-targeting antibodies, PCAD-targeting antibodies such as NOV169N31Q or CQY679, EphA2-targeting antibodies such as EphA2 1C₁, CD48-targeting antibodies such as CD48 NY920 or SGN-CD48A (MEM/MEM102), CD74-targeting antibodies such as milatuzumab or VHmil x VK1aNQ, HER2-targeting antibodies such as trastuzumab or disitamab, TROP2-targeting antibodies such as datopotamab, B7-H3-targeting antibodies such as ABBV-155 or DS-5573a, 5T4-targeting antibodies, and MET-targeting antibodies such as 9006, 9338, and 8902 provided particularly improved drug:antibody ratio, aggregation level, stability (i.e., in vitro and in vivo stability), tumor targeting (i.e., cytotoxicity, potency), minimized off-target killing, and/or treatment efficacy. Improved treatment efficacy can be measured in vitro or in vivo, and may include reduced tumor growth rate and/or reduced tumor volume.

In some embodiments, alternate antibodies to the same targets or antibodies to different antigen targets are used and provide at least some of the favorable functional properties described above (e.g., improved stability, improved tumor targeting, improved treatment efficacy, etc.). In some embodiments, some or all of these favorable functional properties are observed when the disclosed linkers and antineoplastic payloads, such as BH3 mimetics, topoisomerase 1 inhibitors, or anti-mitotic drugs, are conjugated to an alternate TROP2, B7-H3, 5T4, MET, HER2, CD74, CD48, EphA2 or PCAD-targeting antibody or antigen-binding fragment. In other embodiments, some or all of these favorable functional properties are observed when the disclosed linkers and antineoplastic payloads, such as BH3 mimetics, topoisomerase 1 inhibitors, or anti-mitotic drugs, are conjugated to an HER2-targeting antibody or antigen-binding fragment. In some embodiments, the antibody or antigen-binding fragment targets HER2. In some embodiments, the HER2-targeting antibody or antigen-binding fragment is trastuzumab or disitamab. In other embodiments, some or all of these favorable functional properties are observed when the disclosed linkers and antineoplastic payloads, such as BH3 mimetics, topoisomerase 1 inhibitors, or anti-mitotic drugs, are conjugated to an CD74-targeting antibody or antigen-binding fragment. In some embodiments, the antibody or antigen-binding fragment targets CD74. In some embodiments, the CD74-targeting antibody or antigen-binding fragment is milatuzumab. In some embodiments, the CD74-targeting antibody or antigen-binding fragment is VHmil x VK1aNQ. In other embodiments, some or all of these favorable functional properties are observed when the disclosed linkers and antineoplastic payloads, such as BH3 mimetics, topoisomerase 1 inhibitors, or anti-mitotic drugs, are conjugated to an CD48-targeting antibody or antigen-binding fragment. In some embodiments, the antibody or antigen-binding fragment targets CD48. In some embodiments, the CD48-targeting antibody or antigen-binding fragment is CD48 NY920 or SGN-CD48A (MEM/MEM102). In other embodiments, some or all of these favorable functional properties are observed when the disclosed linkers and antineoplastic payloads, such as BH3 mimetics, topoisomerase 1 inhibitors, or anti-mitotic drugs, are conjugated to an EphA2-targeting antibody or antigen-binding fragment. In some embodiments, the antibody or antigen-binding fragment targets EphA2. In some embodiments, the EphA2-targeting antibody or antigen-binding fragment is anti-EphA2 1C₁. In other embodiments, some or all of these favorable functional properties are observed when the disclosed linkers and antineoplastic payloads, such as BH3 mimetics, topoisomerase 1 inhibitors, or anti-mitotic drugs, are conjugated to an PCAD-targeting antibody or antigen-binding fragment. In some embodiments, the antibody or antigen-binding fragment targets PCAD. In some embodiments, the PCAD -targeting antibody or antigen-binding fragment is NOV169N31Q or CQY679. In other embodiments, some or all of these favorable functional properties are observed when the disclosed linkers and antineoplastic payloads, such as BH3 mimetics, topoisomerase 1 inhibitors, or anti-mitotic drugs, are conjugated to an TROP2-targeting antibody or antigen-binding fragment. In some embodiments, the antibody or antigen-binding fragment targets TROP2. In some embodiments, the TROP2-targeting antibody or antigen-binding fragment is datopotamab. In other embodiments, some or all of these favorable functional properties are observed when the disclosed linkers and antineoplastic payloads, such as BH3 mimetics, topoisomerase 1 inhibitors, or anti-mitotic drugs, are conjugated to an B7-H3-targeting antibody or antigen-binding fragment. In some embodiments, the antibody or antigen-binding fragment targets B7-H3. In some embodiments, the B7-H3-targeting antibody or antigen-binding fragment is ABBV-155 or DS-5573a. In other embodiments, some or all of these favorable functional properties are observed when the disclosed linkers and antineoplastic payloads, such as BH3 mimetics, topoisomerase 1 inhibitors, or anti-mitotic drugs, are conjugated to an 5T4-targeting antibody or antigen-binding fragment. In some embodiments, the antibody or antigen-binding fragment targets 5T4. In other embodiments, some or all of these favorable functional properties are observed when the disclosed linkers and antineoplastic payloads, such as BH3 mimetics, topoisomerase 1 inhibitors, or anti-mitotic drugs, are conjugated to an MET-targeting antibody or antigen-binding fragment. In some embodiments, the antibody or antigen-binding fragment targets MET. In some embodiments, the MET-targeting antibody or antigen-binding fragment is 9006, 9338, or 8902.

### B. Linkers

In some embodiments, the linker in an ADC is stable extracellularly in a sufficient manner to be therapeutically effective. In some embodiments, the linker is stable outside a cell, such that the ADC remains intact when present in extracellular conditions (e.g., prior to transport or delivery into a cell). The term “intact,” used in the context of an ADC, means that the antibody or antigen-binding fragment remains attached to the drug moiety (e.g., the antineoplastic payloads, such as BH3 mimetics, topoisomerase 1 inhibitors, or anti-mitotic drugs).

As used herein, “stable,” in the context of a linker or ADC comprising a linker, means that no more than 20%, no more than about 15%, no more than about 10%, no more than about 5%, no more than about 3%, or no more than about 1% of the linkers (or any percentage in between) in a sample of ADC are cleaved (or in the case of an overall ADC are otherwise not intact) when the ADC is present in extracellular conditions. In some embodiments, the linkers and/or ADCs disclosed herein are stable compared to alternate linkers and/or ADCs with alternate linkers and/or antineoplastic payloads, such as BH3 mimetics, topoisomerase 1 inhibitors, or anti-mitotic drugs. In some embodiments, the ADCs disclosed herein can remain intact for more than about 48 hours, more than 60 hours, more than about 72 hours, more than about 84 hours, or more than about 96 hours.

Whether a linker is stable extracellularly can be determined, for example, by including an ADC in plasma for a predetermined time period (e.g., 2, 4, 6, 8, 16, 24, 48, or 72 hours) and then quantifying the amount of free drug moiety present in the plasma. Stability may allow the ADC time to localize to target cancer cells and prevent the premature release of the drug moiety, which could lower the therapeutic index of the ADC by indiscriminately damaging both normal and cancer tissues. In some embodiments, the linker is stable outside of a target cell and releases the drug moiety from the ADC once inside of the cell, such that the drug can bind to its target. Thus, an effective linker will: (i) maintain the specific binding properties of the antibody or antigen-binding fragment; (ii) allow delivery, e.g., intracellular delivery, of the drug moiety via stable attachment to the antibody or antigen-binding fragment; (iii) remain stable and intact until the ADC has been transported or delivered to its target site; and (iv) allow for the therapeutic effect, e.g., cytotoxic effect, of the drug moiety after cleavage or alternate release mechanism.

Linkers may impact the physico-chemical properties of an ADC. As many cytotoxic agents are hydrophobic in nature, linking them to the antibody with an additional hydrophobic moiety may lead to aggregation. ADC aggregates are insoluble and often limit achievable drug loading onto the antibody, which can negatively affect the potency of the ADC. Protein aggregates of biologics, in general, have also been linked to increased immunogenicity. As shown below, linkers disclosed herein result in ADCs with low aggregation levels and desirable levels of drug loading.

A linker may be “cleavable” or “non-cleavable” (Ducry and Stump (2010) Bioconjugate Chem. 21:5-13). Cleavable linkers are designed to release the drug moiety (e.g., a antineoplastic payloads, such as BH3 mimetics, topoisomerase 1 inhibitors, or anti-mitotic drugs) when subjected to certain environment factors, e.g., when internalized into the target cell, whereas non-cleavable linkers generally rely on the degradation of the antibody or antigen-binding fragment itself.

The term “alkyl”, as used herein, refers to a straight or branched hydrocarbon chain radical consisting solely of carbon and hydrogen atoms, containing no unsaturation. The term “C₁-C₆alkyl”, as used herein, refers to a straight or branched hydrocarbon chain radical consisting solely of carbon and hydrogen atoms, containing no unsaturation, having from one to six carbon atoms, and which is attached to the rest of the molecule by a single bond. Non-limiting examples of “C₁-C₆alkyl” groups include methyl (a C₁alkyl), ethyl (a C2alkyl), 1-methylethyl (a C₃alkyl), n-propyl (a C₃alkyl), isopropyl (a C₃alkyl), n-butyl (a C₄alkyl), isobutyl (a C₄alkyl), sec-butyl (a C₄alkyl), tert-butyl (a C₄alkyl), n-pentyl (a C₅alkyl), isopentyl (a C₅alkyl), neopentyl (a C₅alkyl) and hexyl (a C₆alkyl).

The term “alkenyl”, as used herein, refers to a straight or branched hydrocarbon chain radical group consisting solely of carbon and hydrogen atoms, containing at least one double bond. The term “C₂—C₆alkenyl”, as used herein, refers to a straight or branched hydrocarbon chain radical group consisting solely of carbon and hydrogen atoms, containing at least one double bond, having from two to six carbon atoms, which is attached to the rest of the molecule by a single bond. Non-limiting examples of “C₂—C₆alkenyl” groups include ethenyl (a C₂alkenyl), prop-i-enyl (a C₃alkenyl), but-i-enyl (a C₄alkenyl), pent-1-enyl (a C₅alkenyl), pent-4-enyl (a C₅alkenyl), penta-i,4-dienyl (a C₅alkenyl), hexa-i-enyl (a C₆alkenyl), hexa-2-enyl (a C₆alkenyl), hexa-3-enyl (a C₆alkenyl), hexa-i-,4-dienyl (a C₆alkenyl), hexa-i-,5-dienyl (a C₆alkenyl) and hexa-2-,4-dienyl (a C₆alkenyl). The term “C₂-C₃alkenyl”, as used herein, refers to a straight or branched hydrocarbon chain radical group consisting solely of carbon and hydrogen atoms, containing at least one double bond, having from two to three carbon atoms, which is attached to the rest of the molecule by a single bond. Non-limiting examples of “C₂-C₃alkenyl” groups include ethenyl (a C₂alkenyl) and prop-i-enyl (a C₃alkenyl).

The term “alkylene”, as used herein, refers to a bivalent straight or branched hydrocarbon chain radical consisting solely of carbon and hydrogen atoms and containing no unsaturation. The term “C₁-C₆alkylene”, as used herein, refers to a bivalent straight or branched hydrocarbon chain radical consisting solely of carbon and hydrogen atoms, containing no unsaturation, having from one to six carbon atoms. Non-limiting examples of “C₁-C₆alkylene” groups include methylene (a C₁alkylene), ethylene (a C2alkylene), 1-methylethylene (a C₃alkylene), n-propylene (a C₃alkylene), isopropylene (a C₃alkylene), n-butylene (a C₄alkylene), isobutylene (a C₄alkylene), sec-butylene (a C₄alkylene), tert-butylene (a C₄alkylene), n-pentylene (a C₅alkylene), isopentylene (a C₅alkylene), neopentylene (a C₅alkylene), and hexylene (a C₆alkylene).

The term “alkenylene”, as used herein, refers to a bivalent straight or branched hydrocarbon chain radical consisting solely of carbon and hydrogen atoms and containing at least one double bond. The term “C₂-C₆alkenylene”, as used herein, refers to a bivalent straight or branched hydrocarbon chain radical group consisting solely of carbon and hydrogen atoms, containing at least one double bond, and having from two to six carbon atoms. Non-limiting examples of “C₂-C₆alkenylene” groups include ethenylene (a C2alkenylene), prop-1-enylene (a C₃alkenylene), but-1-enylene (a C₄alkenylene), pent-1-enylene (a C₅alkenylene), pent-4-enylene (a C₅alkenylene), penta-1,4-dienylene (a C₅alkenylene), hexa-1-enylene (a C₆alkenylene), hexa-2-enylene (a C₆alkenylene), hexa-3-enylene (a C₆alkenylene), hexa-1-,4-dienylene (a C₆alkenylene), hexa-1-,5-dienylene (a C₆alkenylene) and hexa-2-,4-dienylene (a C₆alkenylene). The term “C₂-C₆alkenylene”, as used herein, refers to a bivalent straight or branched hydrocarbon chain radical group consisting solely of carbon and hydrogen atoms, containing at least one double bond, and having from two to three carbon atoms. Non-limiting examples of “C₂-C₃alkenylene” groups include ethenylene (a C2alkenylene) and prop-i-enylene (a C₃alkenylene).

The term “cycloalkyl,” or “C₃-Cₓ cycloalkyl,” as used herein, refers to a saturated, monocyclic, fused bicyclic, fused tricyclic or bridged polycyclic ring system. Non-limiting examples of fused bicyclic or bridged polycyclic ring systems include bicyclo[1.1.1]pentane, bicyclo[2.1.1]hexane, bicyclo[2.2.1]heptane, bicyclo[3.1.1]heptane, bicyclo[3.2.1]octane, bicyclo[2.2.2]octane and adamantanyl. Non-limiting examples monocyclic C₃-C₅ cycloalkyl groups include cyclopropyl, cyclobutyl, cyclopentyl, cyclohexyl, cycloheptyl and cyclooctyl groups.

The term “aryl” as used herein, refers to a phenyl, naphthyl, biphenyl or indenyl group.

The term “heteroaryl” as used herein, refers any mono- or bi-cyclic group composed of from 5 to 10 ring members, having at least one aromatic moiety and containing from 1 to 4 hetero atoms selected from oxygen, sulphur and nitrogen (including quaternary nitrogens).

The term “cycloalkyl” as used herein, refers to any mono- or bi-cyclic non-aromatic carbocyclic group containing from 3 to 10 ring members, which may include fused, bridged or spiro ring systems. Non-limiting examples of fused bicyclic or bridged ring systems include bicyclo[l.1.1]pentane, bicyclo[2.1.1]hexane, bicyclo[2.2.1]heptane, bicyclo[3.1.1]heptane, bicyclo[3.2.1]octane, and bicyclo[2.2.2]octane. Non-limiting examples monocyclic C₃-Cₓ cycloalkyl groups include cyclopropyl, cyclobutyl, cyclopentyl, cyclohexyl, cycloheptyl and cyclooctyl groups.

The term “heterocycloalkyl” means any mono- or bi-cyclic non-aromatic carbocyclic group, composed of from 3 to 10 ring members, and containing from one to 3 hetero atoms selected from oxygen, sulphur, SO, SO₂ and nitrogen, it being understood that bicyclic group may be fused or spiro type. C₃-C₅ heterocycloalkyl refers to heterocycloalkyl having 3 to 8 ring carbon atoms. The heterocycloalkyl can have 4 to 10 ring members.

The term heteroarylene, cycloalkylene, heterocycloalkylene mean a divalent heteroaryl, cycloalkyl and heterocycloalkyl.

The term “haloalkyl,” as used herein, refers to a linear or branched alkyl chain substituted with one or more halogen groups in place of hydrogens along the hydrocarbon chain. Examples of halogen groups suitable for substitution in the haloalkyl group include Fluorine, Bromine, Chlorine, and Iodine. Haloalkyl groups may include substitution with multiple halogen groups in place of hydrogens in an alkyl chain, wherein said halogen groups can be attached to the same carbon or to another carbon in the alkyl chain.

As used herein, the alkyl, alkenyl, alkynyl, alkoxy, amino, aryl, heteroaryl, cycloalkyl, and heterocycloalkyl groups may be optionally substituted by 1 to 4 groups selected from optionally substituted linear or branched (C₁-C₆)alkyl, optionally substituted linear or branched (C₂-C₆)alkenyl group, optionally substituted linear or branched (C₂-C₆)alkynyl group, optionally substituted linear or branched (C₁-C₆)alkoxy, optionally substituted (C₁-C₆)alkyl-S—, hydroxy, oxo (or N-oxide where appropriate), nitro, cyano, —C(O)—OR₀′, —O—C(O)—R₀′, —C(O)—NR₀′R₀″, —NR₀′R₀″, —(C═NR₀′)—OR₀″, linear or branched (C₁-C₆) haloalkyl, trifluoromethoxy, or halogen, wherein R₀′ and R₀″ are each independently a hydrogen atom or an optionally substituted linear or branched (C₁-C₆)alkyl group, and wherein one or more of the carbon atoms of linear or branched (C₁-C₆)alkyl group is optionally deuterated.

The term “polyoxyethylene”, “polyethylene glycol” or “PEG”, as used herein, refers to a linear chain, a branched chain or a star shaped configuration comprised of (OCH₂CH₂) groups. In certain embodiments a polyethylene or PEG group is —(OCH₂CH₂)ₜ*-, where t is 1-40 or 4-40, and where the “-” indicates the end directed toward the self-immolative spacer and the “*-” indicates the point of attachment to a terminal end group R′ where R′ is OH, OCH₃ or OCH₂CH₂C(═O)OH. In other embodiments a polyethylene or PEG group is —(CH₂CH₂O)ₜ*-, where t is 1-40 or 4-40, and where the “-” indicates the end directed toward the self-immolative spacer and the “*-” indicates the point of attachment to a terminal end group R″ where R¹⁷ is H, CH₃ or CH₂CH₂C(═O)OH. For example, the term “PEG12” as used herein means that t is 12.

The term “polyalkylene glycol”, as used herein, refers to a linear chain, a branched chain or a star shaped configuration comprised of (O(CH₂)ₘ)ₙ groups. In certain embodiments a polyethylene or PEG group is —(O(CH₂)ₘ)ₜ*-, where m is 1-10, t is 1-40 or 4-40, and where the “-” indicates the end directed toward the self-immolative spacer and the “*-” indicates the point of attachment to a terminal end group R′ where R′ is OH, OCH₃ or OCH₂CH₂C(═O)OH. In other embodiments a polyethylene or PEG group is —((CH₂)ₘO)ₜ*-, where m is 1-10, t is 1-40 or 4-40, and where the “-” indicates the end directed toward the self-immolative spacer and the “*-” indicates the point of attachment to a terminal end group R″ where R″ is H, CH₃ or CH₂CH₂C(═O)OH.

The term “reactive group”, as used herein, is a functional group capable of forming a covalent bond with a functional group of an antibody, an antibody fragment, or another reactive group attached to an antibody or antibody fragment. Non limiting examples of such functional groups include reactive groups of Table 8 provided herein.

The term “attachment group” or “coupling group”, as used herein, refers to a bivalent moiety which links the bridging spacer to the antibody or fragment thereof. The attachment or coupling group is a bivalent moiety formed by the reaction between a reaction group and a functional group on the antibody or fragment thereof. Non limiting examples of such bivalent moieties include the bivalent chemical moieties given in Table F and Table G provided herein.

The term “attachment point”, as used herein, refers to a location on the linker that is connected to an antibody or an antineoplastic payload. In some embodiments, the location is an atom, such as carbon, nitrogen, sulfur, or oxygen, where the linker connects with an antibody or an antineoplastic payload through a covalent bond.

The term “bridging spacer”, as used herein, refers to one or more linker components which are covalently attached together to form a bivalent moiety which links the branching moiety W to the attachment group. Non-limiting examples of the bridging spacer include groups L1-1, L1-2, L1-3, L1-4, L1-5, and L1-6 described herein.

The term “branching moiety”, as used herein, refers to a chemical moiety that connects with three or more groups in the dual linker of the present disclosure. In some embodiment, the branching moiety is N or CR^{W}; wherein R^{W} is H or C₁₋₆alkyl.

The term “cleavable group”, as used herein, refers to a moiety that can be unstable in vivo. In some embodiments, the “cleavable group” allows for activation of the antineoplastic payloads by cleaving it from the rest of the conjugate. Operatively defined, the linker is preferably cleaved in vivo by the biological environment. The cleavage may come from any process without limitation, e.g., enzymatic, reductive, pH, etc. In one embodiment, the cleavable group is selected so that activation occurs at the desired site of action, which can be a site in or near the target cells (e.g., carcinoma cells) or tissues such as at the site of therapeutic action or antineoplastic payload activity. Such cleavage may be enzymatic and exemplary enzymatically cleavable groups include natural amino acids or peptide sequences that end with a natural amino acid, and are attached at their carboxyl terminus to the linker. In one embodiment, a cleavable group comprises a pyrophosphate group, a phosphate group, a glucuronide group, a peptide group, and/or a self-immolative group.

The term “enzyme cleavage element”, as used herein, comprises an element that is susceptible to enzymatic cleavage. Nonlimiting examples of the enzymatic cleavage include peptidase-induced cleavage, esterase-induced cleavage, glycosidase induced cleavage, phosphodiesterase induced cleavage, phosphatase induced cleavage, protease induced cleavage, or lipase induced cleavage. In some embodiments, the enzyme cleavage element in the present disclosure refers to a dipeptide group that can be cleaved by a peptidase. In some embodiments, the dipeptide group is selected from a group consisting of El-1 and El-2 described herein. In some embodiments, the enzyme cleavage element in the present disclosure comprises a sugar moiety that can be cleaved by a glucosidase, such as a glucuronide group. In some embodiments, the enzyme cleavage element in the present disclosure comprises a phosphate or pyrophosphate moiety that can be cleaved by phosphatases.

In some embodiments, the enzyme cleavage element is represented by

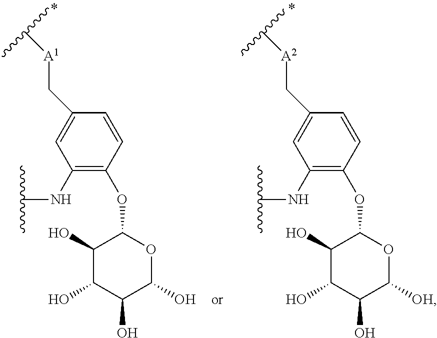

wherein A^{l} and A² are as defined herein, [symbol]
indicates the point of attachment to E¹ or E²; and [symbol] indicates the point of attachment to D¹ or D².

The term “connecting spacer”, as used herein, refers to one or more linker components which are covalently attached together to form a bivalent moiety which links the branching moiety W to the function moiety El or E2 which comprises an enzyme cleavage element or a hydrophilic moiety. Nonlimiting examples of the connecting spacer include groups L2-1 through L2-30 described herein.

The term “hydrophilic group”, as used herein, refers to the group that has hydrophilic properties which increases the aqueous solubility of the dual linker is attached to the linker group of the present disclosure. Examples of such hydrophilic groups include, but are not limited to, polyethylene glycols, polyalkylene glycols, sugars, oligosaccharides, polypeptides, a C₂-C₆alkyl substituted with 1 to 3

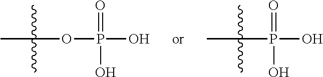 [SMILES: *OP(=O)(O)O ; *P(=O)(O)O]

groups, or C₂-C₆alkyl substituted with 1 to 2 substituents independently selected from —OC(═O)NHS(O)₂NHCH₂CH₂OCH₃, —NHC(═O)C₁₋₄alkylene-P(O)(OCH₂CH₃)₂ and —COOH groups.

The term “hydrophilic moiety”, as used herein, refers to the moiety that comprises a functional group having a hydrophilic group attached thereto. In some embodiments, the functional group mentioned here refers to the bivalent peptide spacer described in the present disclosure.

The term “spacer moiety”, as used herein, refers to one or more linker components which are covalently attached together to form a moiety which links the self-immolative group to the hydrophilic group or an enzyme cleavage element. In some embodiments, the term “spacer moiety”, as used herein, refers to L⁴ or L⁵ as defined herein.

The term “bivalent peptide spacer”, as used herein, refers to bivalent linker comprising one or more amino acid residues covalently attached together to form a moiety which links the bridging spacer to the self immolative spacer or an enzyme cleavage element. The one or more amino acid residues can be an residue of amino acids selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine.

In certain embodiments a “bivalent peptide spacer” is a combination of 2 to four amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu),methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example -ValCit*; -CitVal*; -AlaAla*; —AlaCit*; -CitAla*; -AsnCit*; -CitAsn*; -CitCit*; -ValGlu*; -GluVal*; -SerCit*; -CitSer*; -LysCit*; —CitLys*; -AspCit*; -CitAsp*; -AlaVal*; -ValAla*; -PheAla*; -AlaPhe*; -PheLys*; -LysPhe*; -ValLys*; -LysVal*; -AlaLys*; -LysAla*; -PheCit*; -CitPhe*; -LeuCit*; -CitLeu*; -IleCit*; -CitIle*; -PheArg*; —ArgPhe*; -CitTrp*; -TrpCit*; -PhePheLys*; -LysPhePhe*; -DPhePheLys*; -DLysPhePhe*; —GlyPheLys*; -LysPheGly*; -GlyPheLeuGly- [SEQ ID NO:145]; -GlyLeuPheGly- [SEQ ID NO:146]; —AlaLeuAlaLeu- [SEQ ID NO:147], -GlyGlyGly*; -GlyGlyGlyGly- [SEQ ID NO:148]; -GlyPheValGly-[SEQ ID NO:149]; and -GlyValPheGly- [SEQ ID NO:150], where the “-” indicates the point of attachment to the bridging spacer and the “*” indicates the point of attachment to the self-immolative spacer.

The term “linker component”, as used herein, refers to a chemical moiety that is a part of the linker. Examples of linker components include: an alkylene group: —(CH₂)ₙ— which can either be linear or branched (where in this instance n is 1-18); an alkenylene group; an alkynylene group; an alkenyl group; an alkynyl group; an ethylene glycol unit: —OCH₂CH₂- or —CH₂CH₂O—; an polyethylene glycol unit: (—CH₂CH₂O—)ₓ (where x in this instance is 2-20); —O—; —S—; a carbonyl: —C(═O); an ester: C(═O)—O or O—C(═O); a carbonate: —OC(═O)O—; an amine: —NH—; an tertiary amine; an amide: —C(═O)—NH—, —NH—C(═O)—or —C(═O)N(C₁₋₆alkyl); a carbamate: —OC(═O)NH— or —NHC(═O)O; a urea: —NHC(═O)NH; a sulfonamide: —S(O)₂NH— or —NHS(O)₂;an ether: —CH₂O—or —OCH₂; an alkylene substituted with one or more groups independently selected from carboxy, sulfonate, hydroxyl, amine, amino acid, saccharide, phosphate and phosphonate); an alkenylene substituted with one or more groups independently selected from carboxy, sulfonate, hydroxyl, amine, amino acid, saccharide, phosphate and phosphonate); an alkynylene substituted with one or more groups independently selected from carboxy, sulfonate, hydroxyl, amine, amino acid, saccharide, phosphate and phosphonate); a C₁-C₁₀alkylene in which one or more methylene groups is replace by one or more —S—, —NH— or —O- moieties; a ring systems having two available points of attachment such as a divalent ring selected from phenyl (including 1,2-1,3- and 1,4-di-substituted phenyls), a C₅-C₆ heteroaryl, a C₃-C₈ cycloalkyl (including 1,1-disubstituted cyclopropyl, cyclobutyl, cyclopentyl or cyclohexyl, and 1,4-disubstituted cyclohexyl), and a C₄-C₈ heterocycloalkyl; a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu),methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine; a combination of 2 or more amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu),methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit; and a self-immolative spacer, wherein the self-immolative spacer comprises one or more protecting (triggering) groups which are susceptible to acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.

In addition, a linker component can be a chemical moiety which is readily formed by reaction between two reactive groups. Non-limiting examples of such chemical moieties are given in Table F.

TABLE F

| Reactive Group | Reactive Group | |
|---|---|---|
| 1 | 2 | |
| (RG1) | (RG2) | Chemical Moiety |
| a thiol | a thiol | —S—S— |
| a thiol | a maleimide | 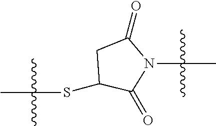 [SMILES: *SC1CC(=O)N(*)C1=O] |
| a thiol | a haloacetamide | 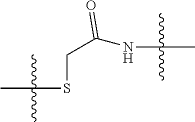 [SMILES: *NC(=O)CS*] |
| an azide | an alkyne | 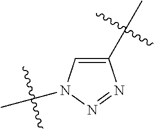 [SMILES: *c1cn(*)nn1] |
| | | or |
| | | 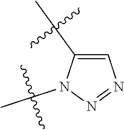 [SMILES: *c1cnnn1*] |
| an azide | a triaryl phosphine | 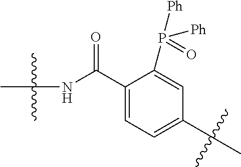 [SMILES: *NC(=O)c1ccc(*)cc1P(=O)(c1ccccc1)c1ccccc1] |
| an azide | a cyclooctyne | 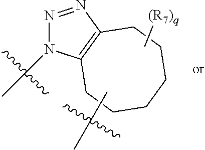 |
| | | 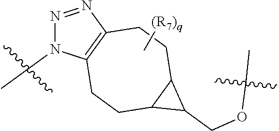 |
| | | or |
| | | 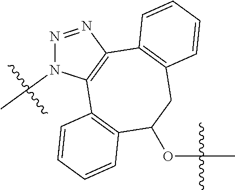 [SMILES: *OC1Cc2ccccc2-c2nnn(*)c2-c2ccccc21] |
| an azide | an oxanobornadiene | 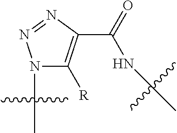 |
| a triaryl phosphine | an azide | 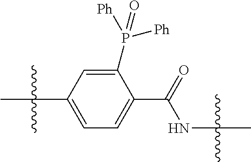 [SMILES: *NC(=O)c1ccc(*)cc1P(=O)(c1ccccc1)c1ccccc1] |
| an oxanobornadiene | an azide | 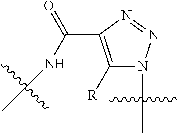 |
| an alkyne | an azide | 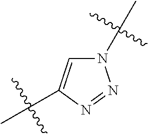 [SMILES: *c1cn(*)nn1] |
| | | or |
| | | 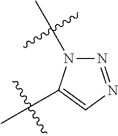 [SMILES: *c1cnnn1*] |
| a cyclooctyne | azide | 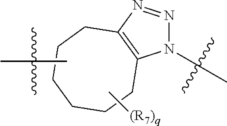 |
| | | or |
| | | 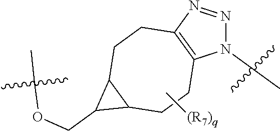 |
| | | or |
| | | 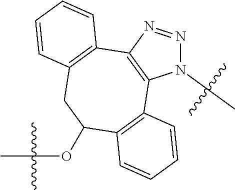 [SMILES: *OC1Cc2ccccc2-c2nnn(*)c2-c2ccccc21] |
| a cyclooctene | a diaryl tetrazine | 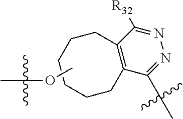 |
| | | or |
| | | 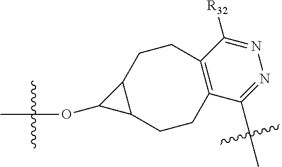 |
| a diaryl tetrazine | a cyclooctene | 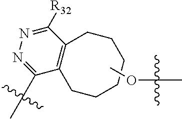 |
| | | or |
| | | 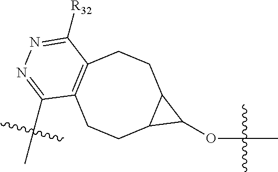 |
| a monoaryl tetrazine | a norbornene | 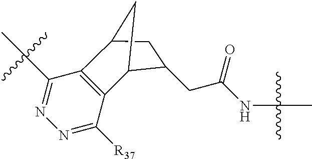 |
| a norbornene | a monoaryl tetrazine | 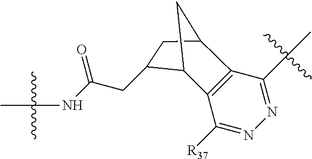 |
| an aldehyde | a hydroxylamine | 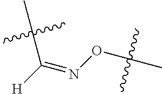 [SMILES: */C=N\O*] |
| an aldehyde | a hydrazine | 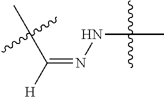 [SMILES: */C=N\N*] |
| an aldehyde | NH₂—NH—C(═O)— | 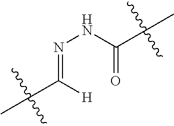 [SMILES: */C=N/NC(*)=O] |
| a ketone | a hydroxylamine | 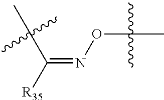 |
| a ketone | a hydrazine | 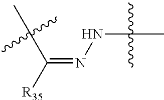 |
| a ketone | NH₂—NH—C(═O)— | 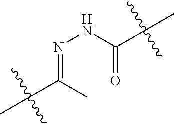 [SMILES: *C(=O)N/N=C(/*)C] |
| a hydroxylamine | an aldehyde | 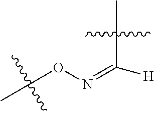 [SMILES: */C=N\O*] |
| a hydroxylamine | a ketone | 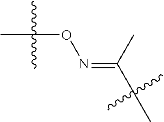 [SMILES: *O/N=C(/*)C] |
| a hydrazine | an aldehyde | 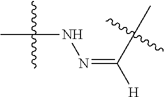 [SMILES: */C=N\N*] |
| a hydrazine | a ketone | 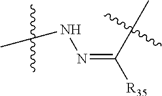 |
| NH₂—NH—C(═O)— | an aldehyde | 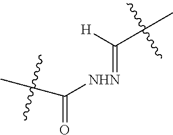 |
| NH₂—NH—C(═O)— | a ketone | 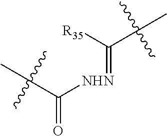 |
| a haloacetamide | a thiol | 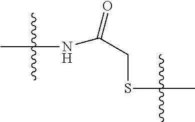 [SMILES: *NC(=O)CS*] |
| a maleimide | a thiol | 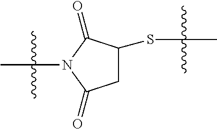 [SMILES: *SC1CC(=O)N(*)C1=O] |
| a vinyl sulfone | a thiol | 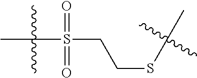 [SMILES: *SCCS(*)(=O)=O] |
| a thiol | a vinyl sulfone | 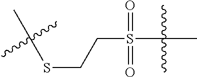 [SMILES: *SCCS(*)(=O)=O] |
| an aziridine | a thiol | 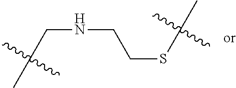 [SMILES: *CNCCS*] |
| | | 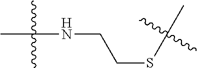 [SMILES: *NCCS*] |
| a thiol | an aziridine | 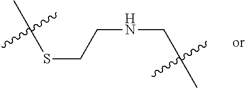 [SMILES: *CNCCS*] |
| | | 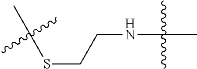 [SMILES: *NCCS*] |
| 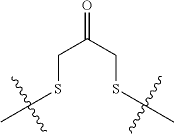 [SMILES: *SCC(=O)CS*] | hydroxylamine | 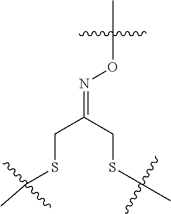 [SMILES: *ON=C(CS*)CS*] |
| 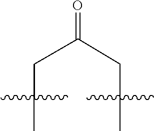 [SMILES: *CC(=O)C*] | hydroxylamine | 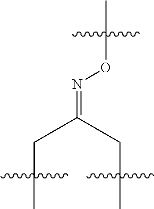 [SMILES: *CC(C*)=NO*] |
| 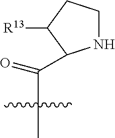 | 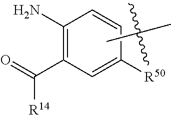 | 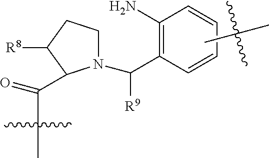 |
| 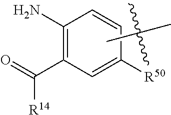 | 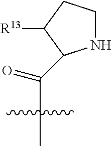 | 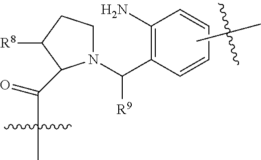 |
| 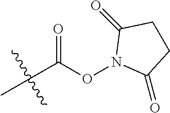 [SMILES: *C(=O)ON1C(=O)CCC1=O] | —NH₂, | amide |
| 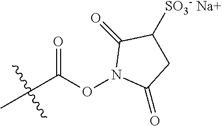 | | |
| 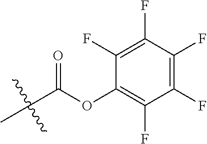 [SMILES: *C(=O)Oc1c(F)c(F)c(F)c(F)c1F] | | |
| 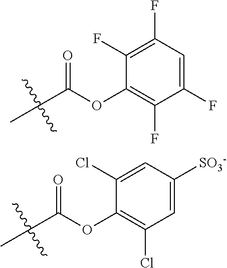 | | |
| —NH₂, | 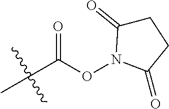 [SMILES: *C(=O)ON1C(=O)CCC1=O] | amide |
| | 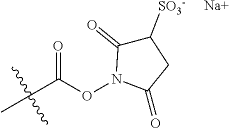 | |
| | 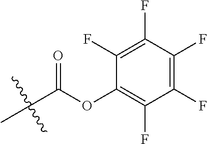 [SMILES: *C(=O)Oc1c(F)c(F)c(F)c(F)c1F] | |
| | 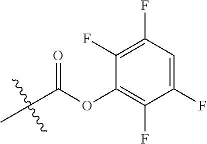 [SMILES: *C(=O)Oc1c(F)c(F)cc(F)c1F] | |
| | 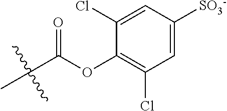 | |
| CoA or CoA analogue | Serine residue | 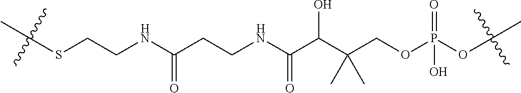 [SMILES: *OP(=O)(O)OCC(C)(C)C(O)C(=O)NCCC(=O)NCCS*] |
| | | 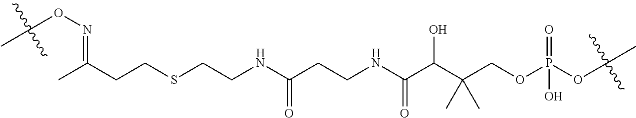 [SMILES: *O/N=C(\C)CCSCCNC(=O)CCNC(=O)C(O)C(C)(C)COP(=O)(O)O*] |
| | | 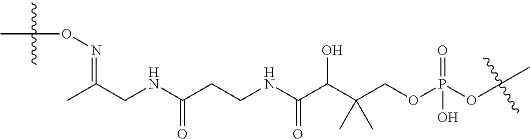 [SMILES: *O/N=C(\C)CNC(=O)CCNC(=O)C(O)C(C)(C)COP(=O)(O)O*] |
| | | 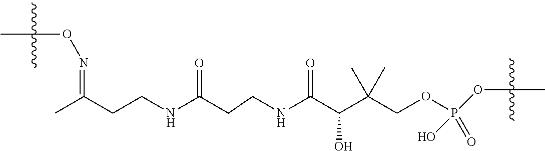 [SMILES: *O/N=C(\C)CCNC(=O)CCNC(=O)[C@@H](O)C(C)(C)COP(=O)(O)O*] |
| | | 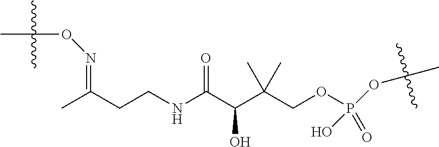 [SMILES: *O/N=C(\C)CCNC(=O)[C@H](O)C(C)(C)COP(=O)(O)O*] |
| | | 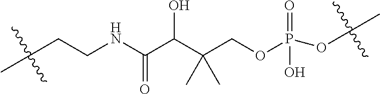 [SMILES: *CCNC(=O)C(O)C(C)(C)COP(=O)(O)O*] |
| | | 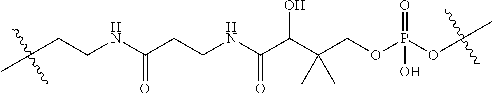 [SMILES: *CCNC(=O)CCNC(=O)C(O)C(C)(C)COP(=O)(O)O*] |
| | | 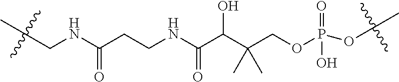 [SMILES: *CNC(=O)CCNC(=O)C(O)C(C)(C)COP(=O)(O)O*] |
| pyridyldithiol | thiol | disulfide |

where: R³² in Table F is H, C₁0.4 alkyl, phenyl, pyrimidine or pyridine; R^{L45} in Table F is H, C₁-6alkyl, phenyl or C₁₋₄alkyl substituted with 1 to 3 —OH groups; each R⁷ in Table F is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH; R³⁷ in Table F is independently selected from H, phenyl and pyridine; q in Table F is 0, 1, 2 or 3; R¹ and R¹³ in Table F is H or methyl; and R⁹ and R¹⁴ in Table F is H, —CH₃ or phenyl; R in Table F is H or any suitable substituent; and R⁵⁰ in Table F is H.

In addition, a linker component can be a group listed in Table G below.

TABLE G

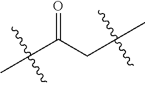 [SMILES: *CC(*)=O]

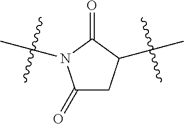 [SMILES: *C1CC(=O)N(*)C1=O]

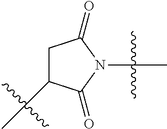 [SMILES: *C1CC(=O)N(*)C1=O]

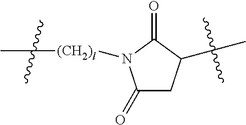

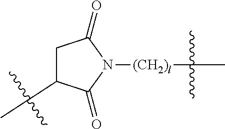

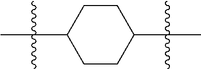 [SMILES: *C1CCC(*)CC1]

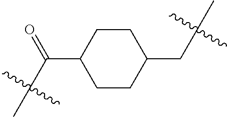 [SMILES: *CC1CCC(C(*)=O)CC1]

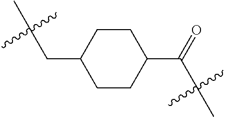 [SMILES: *CC1CCC(C(*)=O)CC1]

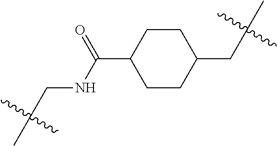 [SMILES: *CNC(=O)C1CCC(C*)CC1]

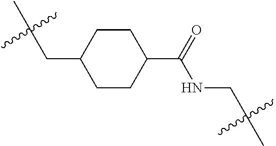 [SMILES: *CNC(=O)C1CCC(C*)CC1]

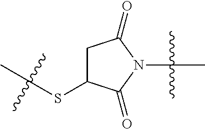 [SMILES: *SC1CC(=O)N(*)C1=O]

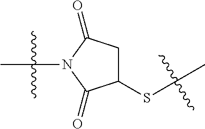 [SMILES: *SC1CC(=O)N(*)C1=O]

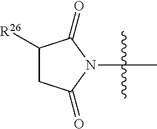

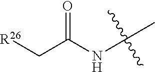

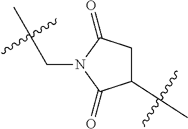 [SMILES: *CN1C(=O)CC(*)C1=O]

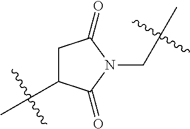 [SMILES: *CN1C(=O)CC(*)C1=O]

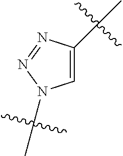 [SMILES: *c1cn(*)nn1]

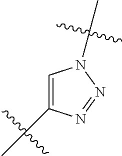 [SMILES: *c1cn(*)nn1]

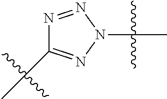 [SMILES: *c1nnn(*)n1]

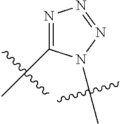 [SMILES: *c1nnnn1*]

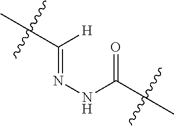 [SMILES: */C=N/NC(*)=O]

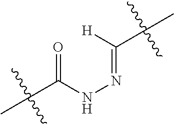 [SMILES: */C=N/NC(*)=O]

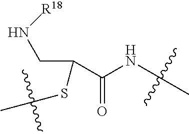

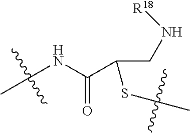

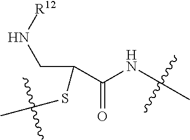

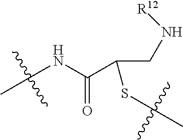

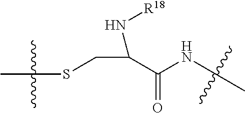

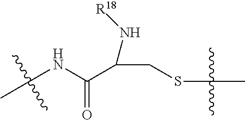

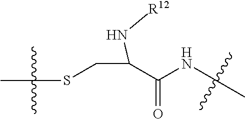

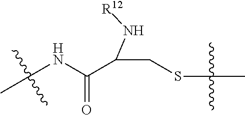

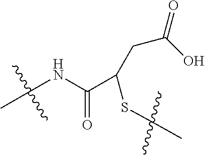 [SMILES: *NC(=O)C(CC(=O)O)S*]

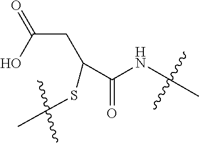 [SMILES: *NC(=O)C(CC(=O)O)S*]

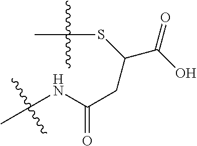 [SMILES: *NC(=O)CC(S*)C(=O)O]

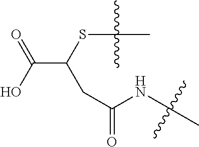 [SMILES: *NC(=O)CC(S*)C(=O)O]

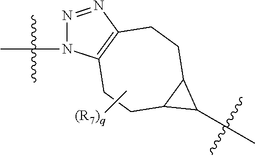

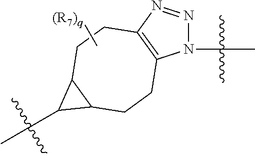

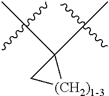 [SMILES: *C1(*)CC1]

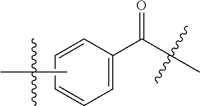

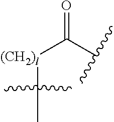

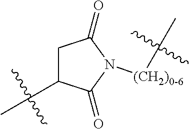 [SMILES: *CN1C(=O)CC(*)C1=O]

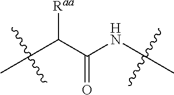

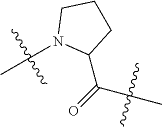 [SMILES: *C(=O)C1CCCN1*]

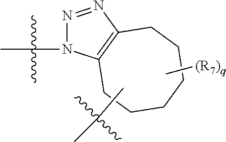

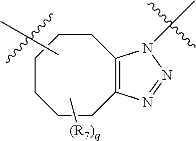

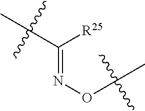

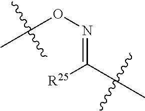

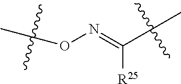

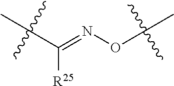

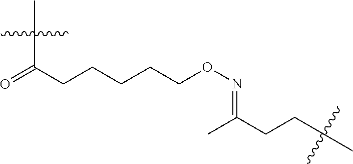 [SMILES: *CC/C(C)=N/OCCCCCC(*)=O]

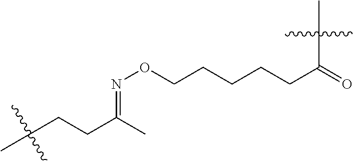 [SMILES: *CC/C(C)=N/OCCCCCC(*)=O]

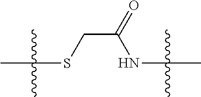 [SMILES: *NC(=O)CS*]

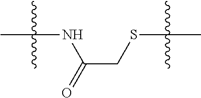 [SMILES: *NC(=O)CS*]

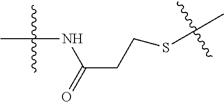 [SMILES: *NC(=O)CCS*]

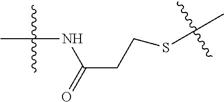 [SMILES: *NC(=O)CCS*]

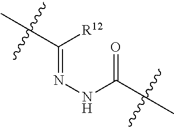

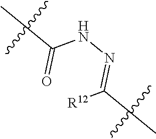

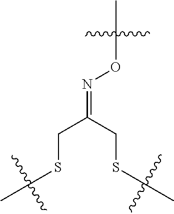 [SMILES: *ON=C(CS*)CS*]

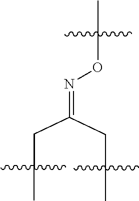 [SMILES: *CC(C*)=NO*]

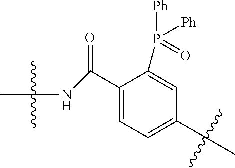 [SMILES: *NC(=O)c1ccc(*)cc1P(=O)(c1ccccc1)c1ccccc1]

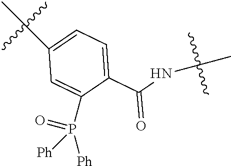 [SMILES: *NC(=O)c1ccc(*)cc1P(=O)(c1ccccc1)c1ccccc1]

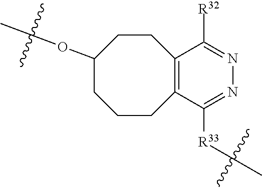

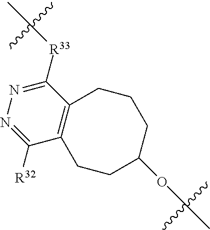

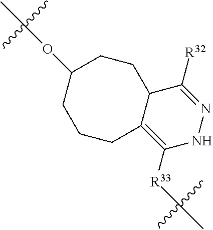

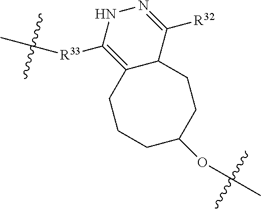

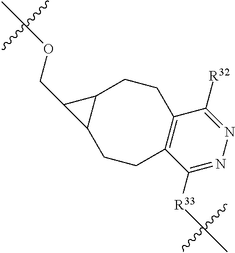

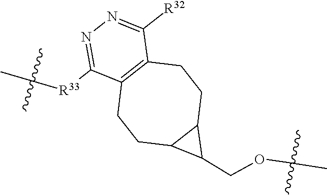

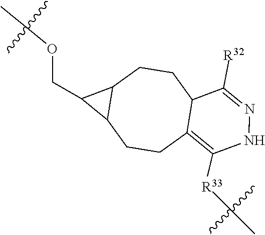

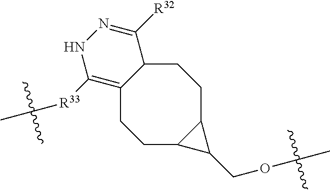

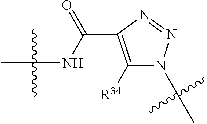

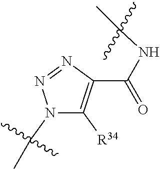

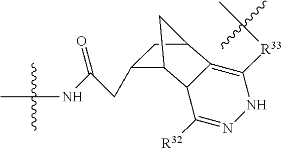

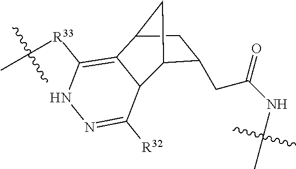

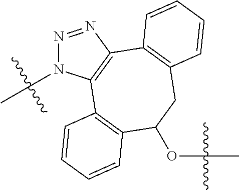 [SMILES: *OC1Cc2ccccc2-c2nnn(*)c2-c2ccccc21]

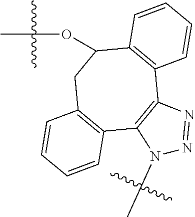 [SMILES: *OC1Cc2ccccc2-c2c(nnn2*)-c2ccccc21]

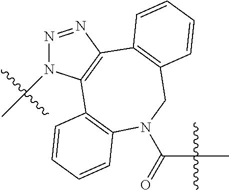 [SMILES: *C(=O)N1Cc2ccccc2-c2nnn(*)c2-c2ccccc21]

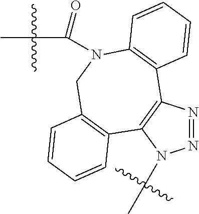 [SMILES: *C(=O)N1Cc2ccccc2-c2c(nnn2*)-c2ccccc21]

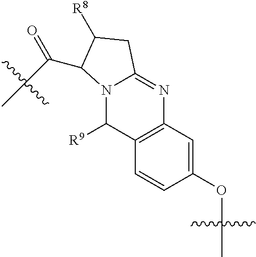

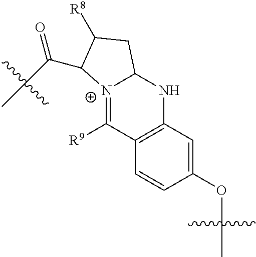

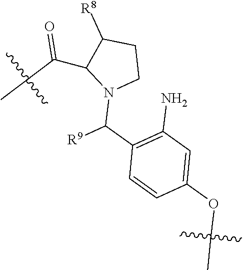

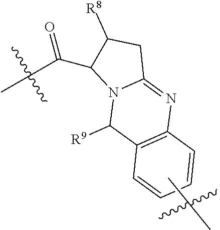

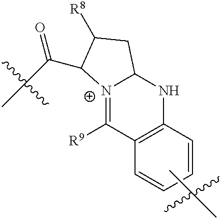

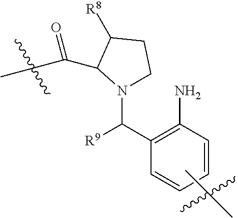

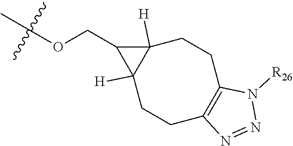

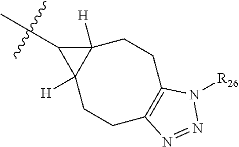

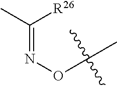

each R⁷ is independently selected from H, C₁₋₆alkyl, fluoro, benzyloxy substituted with —C(═O)OH,
benzyl substituted with —C(═O)OH, C₁₋₄alkoxy substituted with —C(═O)OH and C₁₋₄alkyl
substituted with —C(═O)OH;
each R¹² is independently selected from H and C₁₋C₆alkyl
R⁸ is H or methyl;
R⁹ is H, —CH₃ or phenyl;
each R²⁵ is independently selected from H or C₁₋₄ alkyl;
each R¹⁸ is independently selected from a C₁₋C₆alkyl, a C₁₋C₆alkyl which is substituted with azido
and a C₁₋C₆alkyl which is substituted with 1 to 5 hydroxyl;
q is 0, 1, 2 or 3;
l is 1, 2, 3, 4, 5 or 6;
R²⁶ is

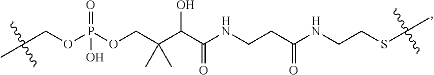 [SMILES: *COP(=O)(O)OCC(C)(C)C(O)C(=O)NCCC(=O)NCCS*]

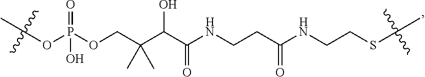 [SMILES: *OP(=O)(O)OCC(C)(C)C(O)C(=O)NCCC(=O)NCCS*]

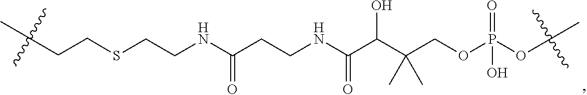 [SMILES: *CCSCCNC(=O)CCNC(=O)C(O)C(C)(C)COP(=O)(O)O*]

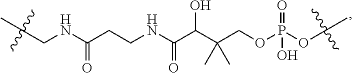 [SMILES: *CNC(=O)CCNC(=O)C(O)C(C)(C)COP(=O)(O)O*]

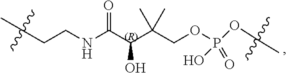

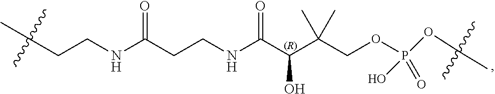

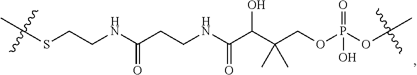 [SMILES: *OP(=O)(O)OCC(C)(C)C(O)C(=O)NCCC(=O)NCCS*]

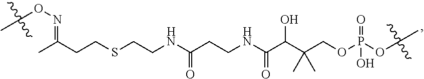 [SMILES: *O/N=C(\C)CCSCCNC(=O)CCNC(=O)C(O)C(C)(C)COP(=O)(O)O*]

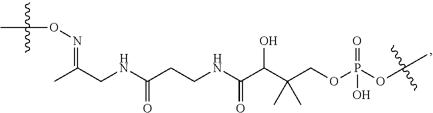 [SMILES: *O/N=C(\C)CNC(=O)CCNC(=O)C(O)C(C)(C)COP(=O)(O)O*]

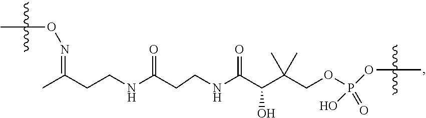 [SMILES: *O/N=C(\C)CCNC(=O)CCNC(=O)[C@@H](O)C(C)(C)COP(=O)(O)O*]

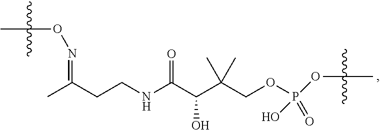 [SMILES: *O/N=C(\C)CCNC(=O)[C@@H](O)C(C)(C)COP(=O)(O)O*]

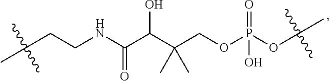 [SMILES: *CCNC(=O)C(O)C(C)(C)COP(=O)(O)O*]

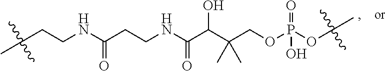 [SMILES: *CCNC(=O)CCNC(=O)C(O)C(C)(C)COP(=O)(O)O*]

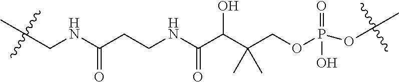 [SMILES: *CNC(=O)CCNC(=O)C(O)C(C)(C)COP(=O)(O)O*]

R³² is independently selected from H, C₁₋₄ alkyl, phenyl, pyrimidine and pyridine;
R³³ is independently selected from

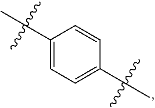 [SMILES: *c1ccc(*)cc1]

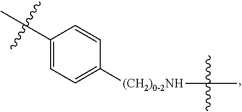

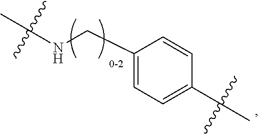

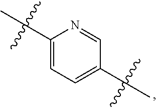 [SMILES: *c1ccc(*)nc1]

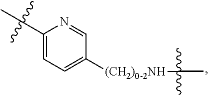

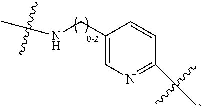

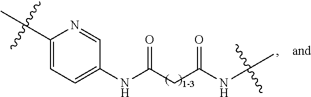

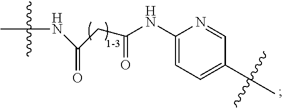

R³⁴ is independently selected from H, C₁₋₄ alkyl, and C₁₋₆ haloalkyl, and
R^{aa} is an amino acid side chain.

As used herein, when a partial structure of a compound is illustrated, a wavy line ( [symbol] ) indicates the point of attachment of the partial structure to the rest of the molecule.

The term “self-immolative spacer”, as used herein, refers to a moiety comprising one or more triggering groups (TG) which are activated by acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage, and after activation the protecting group is removed, which generates a cascade of disassembling reactions leading to the temporally sequential release of a leaving group. Such cascade of reactions can be, but not limited to, 1,2-, 1,4-, 1,6- or 1,8- elimination reactions.

Non-limiting examples of self-immolative spacer include:
TG-Xₐ-LG, TG-Yₐ-LG

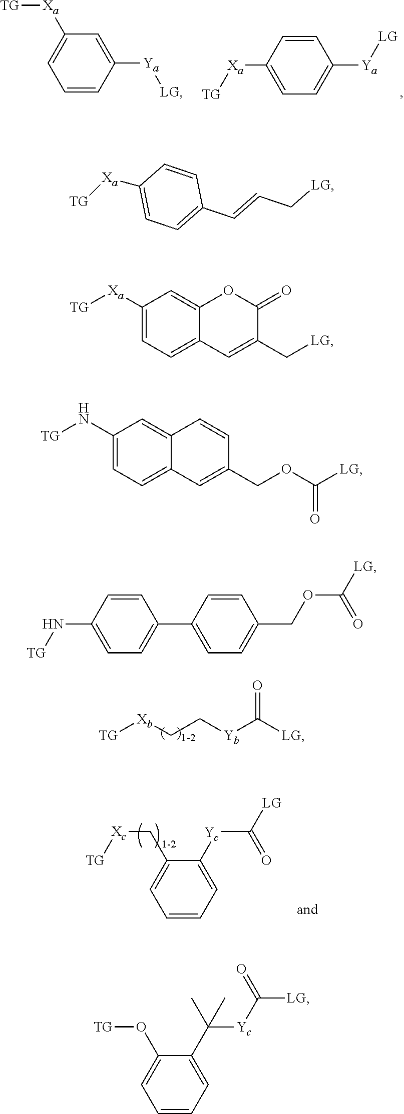

wherein such groups can be optionally substituted, and
wherein:
   TG is a triggering group;
   Xₐ is O, NH or S;
   X_{b} is O, NH, NCH₃ or S;
   X_{c} is O or NH;
   Yₐ is CH₂, CH₂O or CH₂NH;
   Yb is CH₂, O or NH;
   Y_{c} is a bond, CH₂, O or NH, and
   LG is a leaving group such as a Drug moiety (D) of the Linker-Drug group of the invention.

Additional non-limiting examples of self-immolative spacers are described in Angew. Chem. Int. Ed. 2015, 54, 7492-7509.

In certain embodiment the self-immolative spacer connected to a drug moiety is a moiety having the following structure:

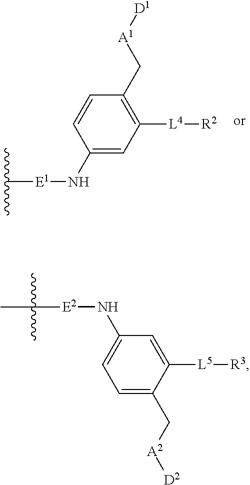

wherein E¹ and/or E² is an enzyme cleavage element, and A¹, A², D¹, D², R², R³, L³, and L⁴ are as defined herein.

In certain embodiment the self-immolative spacer is moiety having the structure

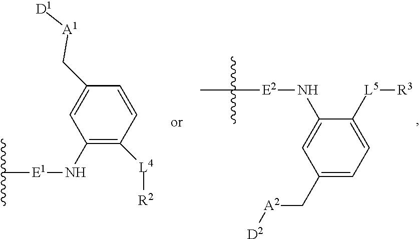

wherein E¹ and/or E² is a bivalent peptide spacer, R² and/or R³ is an enzyme cleavage element, and A¹, A², D¹, D², L³, and L⁴ are as defined herein.

The term “self-immolative group,” as used herein, refer to a group that can generate a cascade of disassembling reactions leading to the temporally sequential release of a leaving group when the TG is activated and removed.

In some embodiments, the self-immolative group is a group having the structure

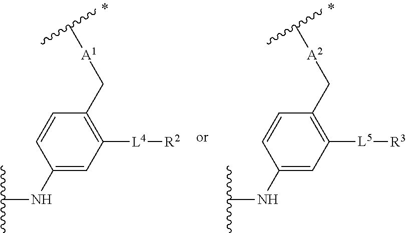

wherein A¹, A², R², R³, L³, and L⁴ are as defined herein, [symbol] indicates the point of attachment to E¹ or E²; and [symbol] indicates the point of attachment to D¹ or D².

### C. Drug Moieties

In some embodiments, an intermediate, which is the precursor of the linker moiety, is reacted with the drug moiety (e.g., BH3 mimetics, such as a Mcl-1 inhibitor, a Bcl-2 inhibitor and/or a Bcl-xL inhibitor; topoisomerase 1 inhibitors, such as topotecan, exatecan, deruxtecan or SN-38; or anti-mitotic drugs, such as monomethyl auristatin E (MMAE) or a taxane) under appropriate conditions. In some embodiments, reactive groups are used on the drug and/or the intermediate or linker. The product of the reaction between the drug and the intermediate, or the derivatized drug (drug plus linker), is subsequently reacted with the antibody or antigen-binding fragment under conditions that facilitate conjugation of the drug and intermediate or derivatized drug and antibody or antigen-binding fragment. Alternatively, the intermediate or linker may first be reacted with the antibody or antigen-binding fragment, or a derivatized antibody or antigen-binding fragment, and then reacted with the drug or derivatized drug.

A number of different reactions are available for covalent attachment of the drug moiety and/or linker moiety to the antibody or antigen-binding fragment. This is often accomplished by reaction of one or more amino acid residues of the antibody or antigen-binding fragment, including the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a drug moiety to an amino (or carboxy) group on an antibody or antigen-binding fragment. Additionally, bifunctional agents such as dialdehydes or imidoesters may also be used to link the amino group on a drug moiety to an amino group on an antibody or antigen-binding fragment. Also available for attachment of drugs (e.g., a BH3 mimetic, a topoisomerase 1 inhibitor, or an anti-mitotic drug) to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure. Examples of drug moieties that can be generated and linked to an antibody or antigen-binding fragment using various chemistries known to in the art include Mcl-1 inhibitors, Bcl-2 inhibitors, and Bcl-xL inhibitors, e.g., the Mcl-1 inhibitors, Bcl-2 inhibitors, and Bcl-xL inhibitors described and exemplified herein. Additional examples of drug moieties that can be generated and linked to an antibody or antigen-binding fragment using various chemistries known to in the art include topoisomerase 1 inhibitors or anti-mitotic drugs described and exemplified herein.

a. Mcl-1 inhibitors

Suitable BH3 mimetic D¹ and/or D² may comprise a Mcl-1 inhibitor compound of the formulas (I), (IA), (IB), or an enantiomer, diastereoisomer, atropisomer, deuterated derivative, and/or addition salt thereof with a pharmaceutically acceptable acid or base. Additionally, the drug moiety may comprise any compounds of the Mcl-1 inhibitor (D) described herein.

As used herein, “atropisomers,” are stereoisomers arising because of hindered rotation about a single bond, where energy differences due to steric strain or other contributors create a barrier to rotation that is high enough to allow for isolation of individual conformers (Bringmann et al. Angew. Chem. Int. Ed. 2005, 44, 5384-5427). For example, for compounds of formula (II) according to the invention, atropisomers may be as follows:

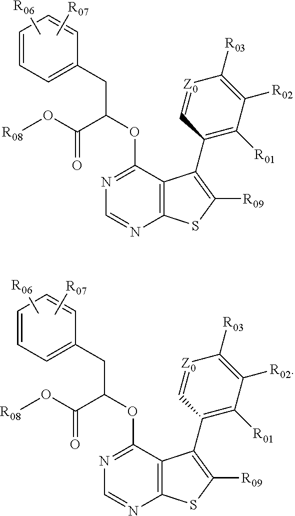

For example, a preferred atropisomer may be (5Sa), also named (5aS).

A drug moiety of the disclosure may be any one of the compounds disclosed in International Patent Application Publication Nos. WO 2015/097123; WO 2016/207216; WO 2016/207217; WO 2016/207225; WO 2016/207226; WO 2017/125224; WO 2019/035899; WO 2019/035911; WO 2019/035914; WO 2019/035927; WO 2016/033486; WO 2017/147410; WO 2018/183418; and WO 2017/182625, and U.S. Patent Application Publication No. 2019/0055264, each of which is incorporated herein by reference in its entirety.

In some embodiments, BH3 mimetics of the disclosure may comprise a compound of Formula (I), (IA) or (IB), wherein the definitions of the variables depicted therein are described above

In some embodiments, Cy₀₁, Cy₀₂, Cy₀₃, Cy₀₄, Cy₀₅, Cy₀₆, Cy₀₇, Cy₀₈ and Cy₀₁₀ independently of one another, are an optionally substituted cycloalkyl group, an optionally substituted heterocycloalkyl group, an optionally substituted aryl group or an optionally substituted heteroaryl group, wherein the optional substituents are selected from optionally substituted linear or branched (C₁-C₆)alkyl, optionally substituted linear or branched (C₂-C₆)alkenyl group, optionally substituted linear or branched (C₂-C₆)alkynyl group, optionally substituted linear or branched (C₁-C₆)alkoxy, optionally substituted (C₁-C₆)alkyl-S—, hydroxy, oxo (or N-oxide where appropriate), nitro, cyano, —C(O)—OR₀′, —O—C(O)—R₀′, —C(O)—NR₀′R₀″, —NR₀′R₀″, -(C═NR₀′)—OR₀″, linear or branched (C₁-C₆)haloalkyl, trifluoromethoxy, or halogen, wherein R₀′ and R₀″ are each independently a hydrogen atom or an optionally substituted linear or branched (C₁-C₆)alkyl group, and wherein one or more of the carbon atoms of linear or branched (C₁-C₆)alkyl group is optionally deuterated.

In some embodiments, BH3 mimetics D¹ and/or D² of the disclosure comprise:

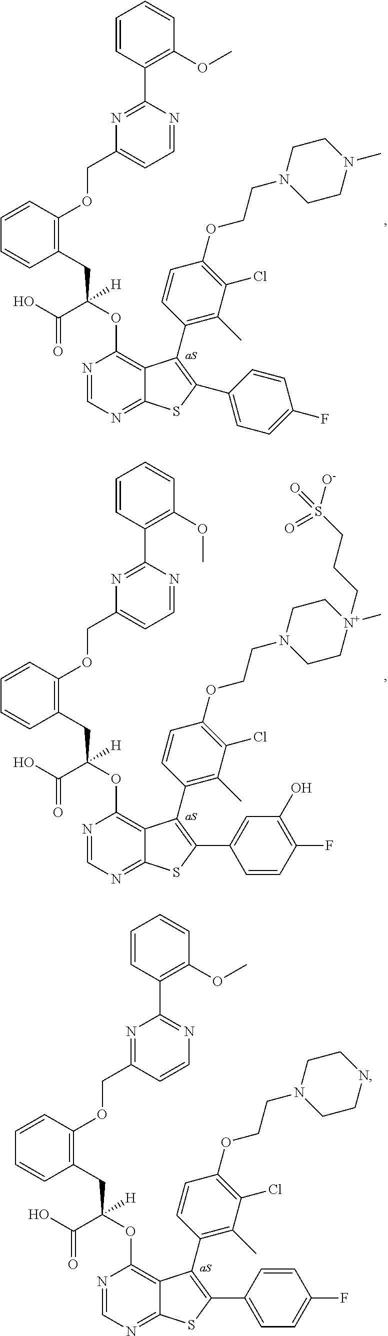

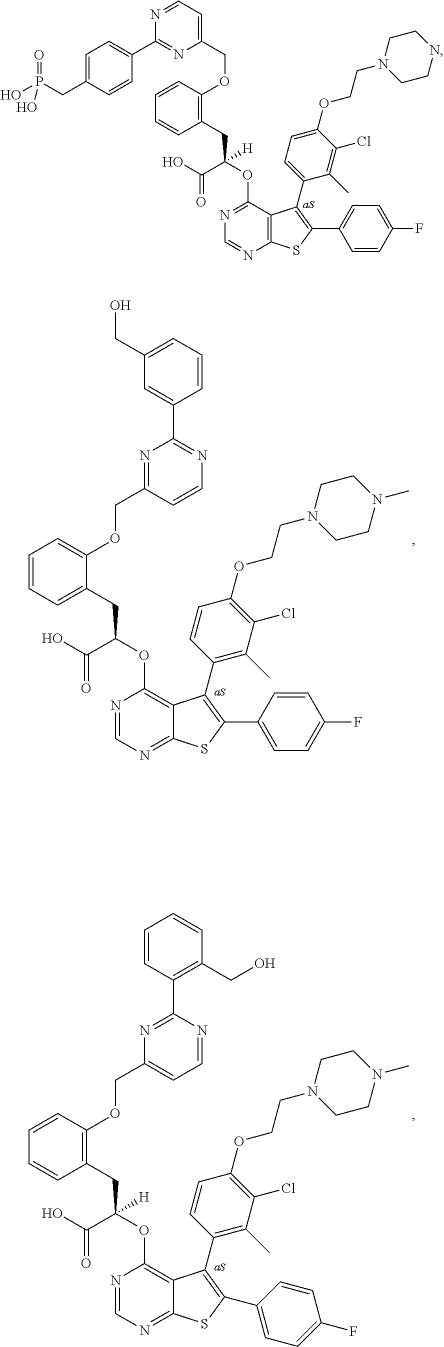

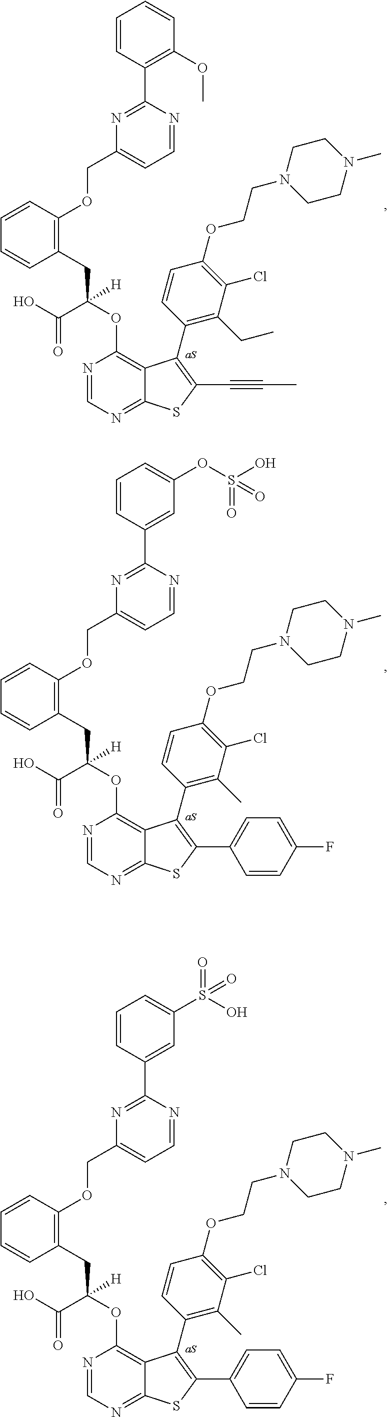 [SMILES: CC#Cc1sc2ncnc(O[C@H](Cc3ccccc3OCc3ccnc(-c4ccccc4OC)n3)C(=O)O)c2c1-c1ccc(OCCN2CCN(C)CC2)c(Cl)c1CC ; Cc1c(-c2c(-c3ccc(F)cc3)sc3ncnc(O[C@H](Cc4ccccc4OCc4ccnc(-c5cccc(OS(=O)(=O)O)c5)n4)C(=O)O)c23)ccc(OCCN2CCN(C)CC2)c1Cl ; Cc1c(-c2c(-c3ccc(F)cc3)sc3ncnc(O[C@H](Cc4ccccc4OCc4ccnc(-c5cccc(S(=O)(=O)O)c5)n4)C(=O)O)c23)ccc(OCCN2CCN(C)CC2)c1Cl]

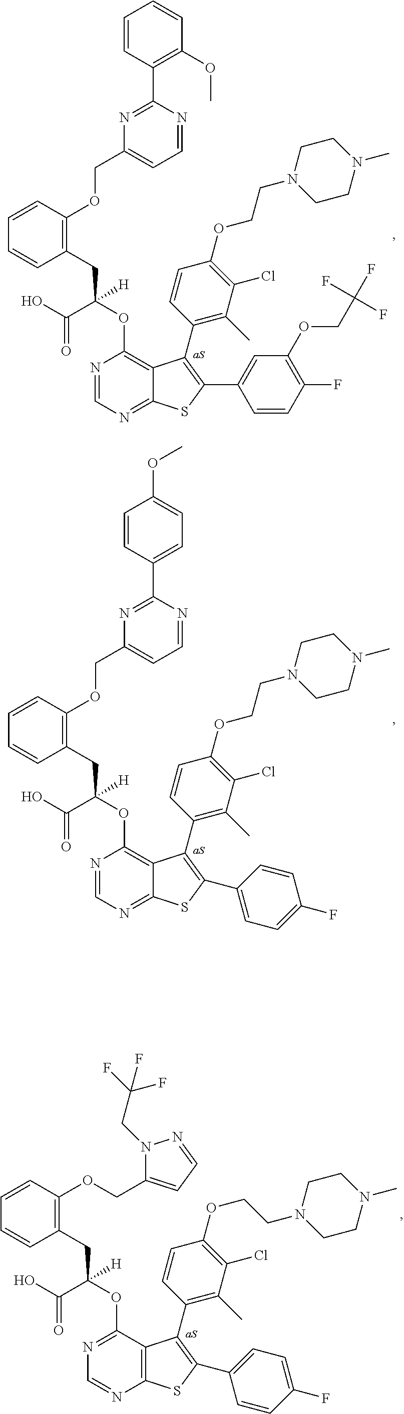

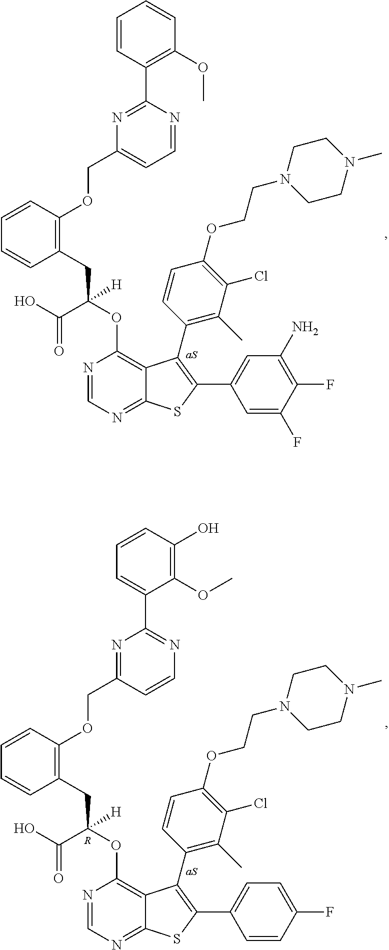

or an enantiomer, diastereoisomer, atropisomer, deuterated derivative, and/or a pharmaceutically acceptable salt of any of the foregoing.

Additionally, BH3 mimetics D¹ and/or D² of the disclosure may comprise any one of the following:

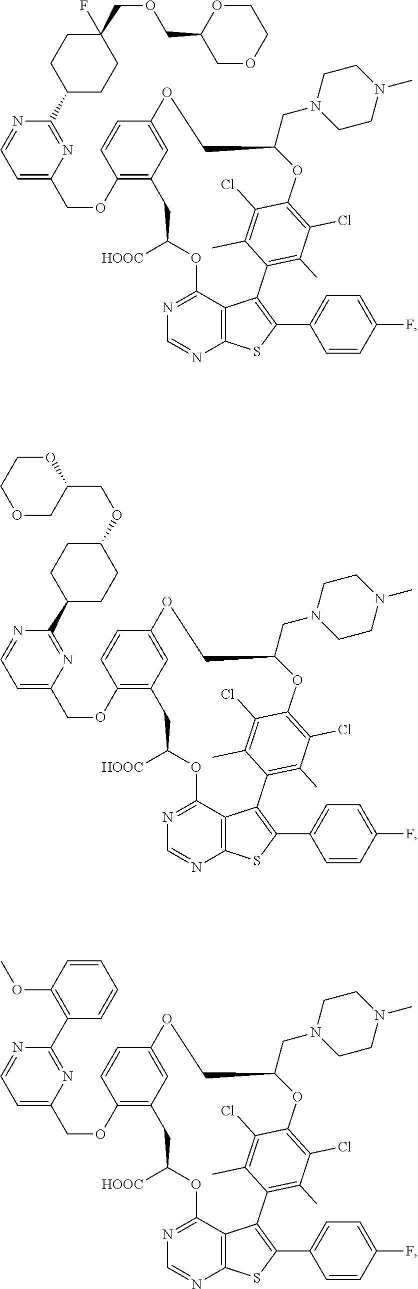 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc([C@H]3CC[C@](F)(COC[C@@H]4COCCO4)CC3)n1)OC[C@@H](CN1CCN(C)CC1)O2 ; Cc1c(Cl)c2c(Cl)c(C)c1-c1c(-c3ccc(F)cc3)sc3ncnc(c13)O[C@@H](C(=O)O)Cc1cc(ccc1OCc1ccnc([C@H]3CC[C@H](OC[C@@H]4COCCO4)CC3)n1)OC[C@@H](CN1CCN(C)CC1)O2 ; COc1ccccc1-c1nccc(COc2ccc3cc2C[C@H](C(=O)O)Oc2ncnc4sc(-c5ccc(F)cc5)c(c24)-c2c(C)c(Cl)c(c(Cl)c2C)O[C@H](CN2CCN(C)CC2)CO3)n1]

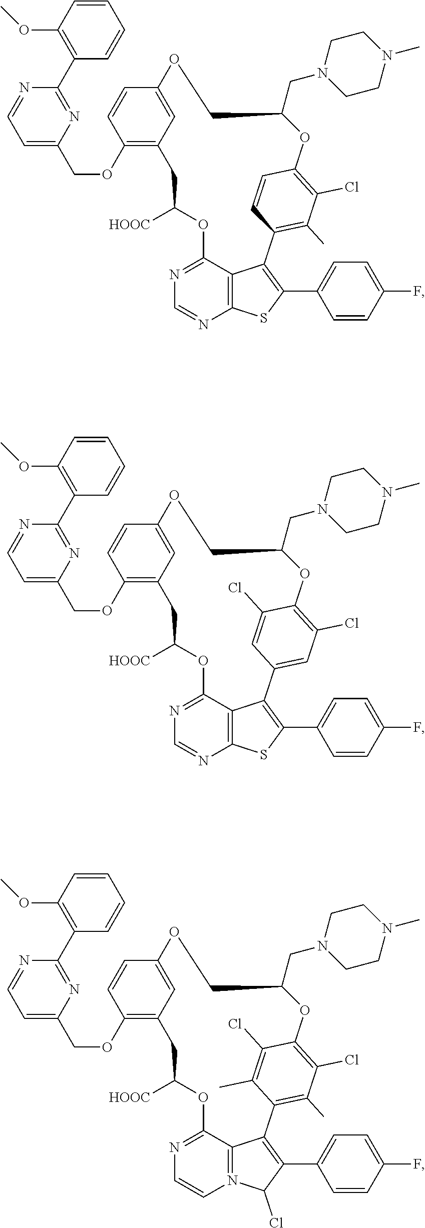

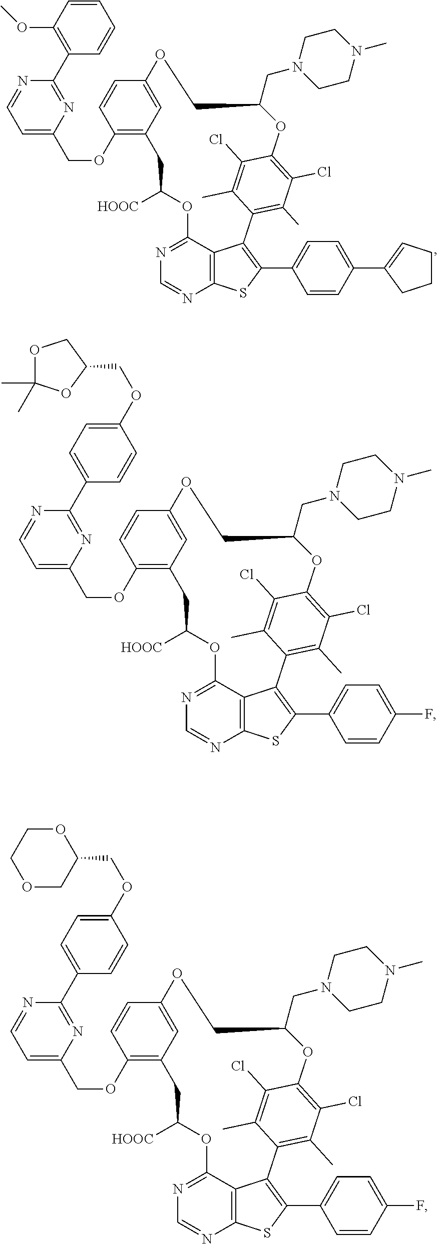

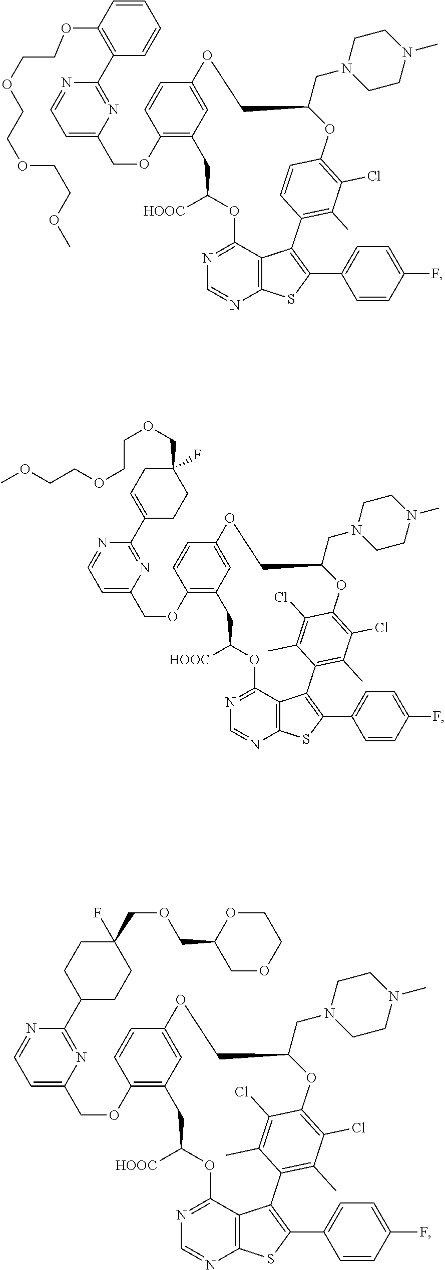

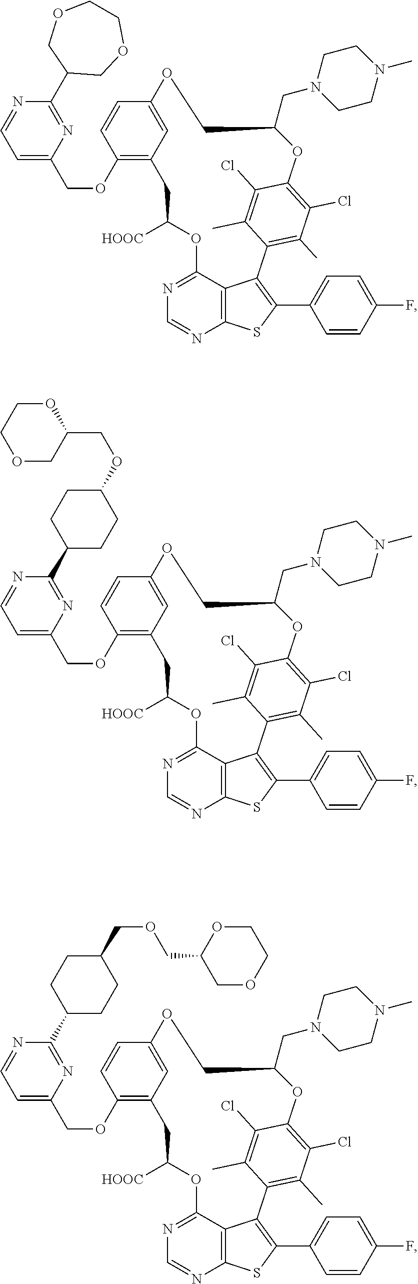 [SMILES: Cc1c(Cl)c2c(Cl)c(C)c1-c1c(-c3ccc(F)cc3)sc3ncnc(c13)O[C@@H](C(=O)O)Cc1cc(ccc1OCc1ccnc(C3COCCOC3)n1)OC[C@@H](CN1CCN(C)CC1)O2 ; Cc1c(Cl)c2c(Cl)c(C)c1-c1c(-c3ccc(F)cc3)sc3ncnc(c13)O[C@@H](C(=O)O)Cc1cc(ccc1OCc1ccnc([C@H]3CC[C@H](OC[C@@H]4COCCO4)CC3)n1)OC[C@@H](CN1CCN(C)CC1)O2 ; Cc1c(Cl)c2c(Cl)c(C)c1-c1c(-c3ccc(F)cc3)sc3ncnc(c13)O[C@@H](C(=O)O)Cc1cc(ccc1OCc1ccnc([C@H]3CC[C@H](COC[C@H]4COCCO4)CC3)n1)OC[C@@H](CN1CCN(C)CC1)O2]

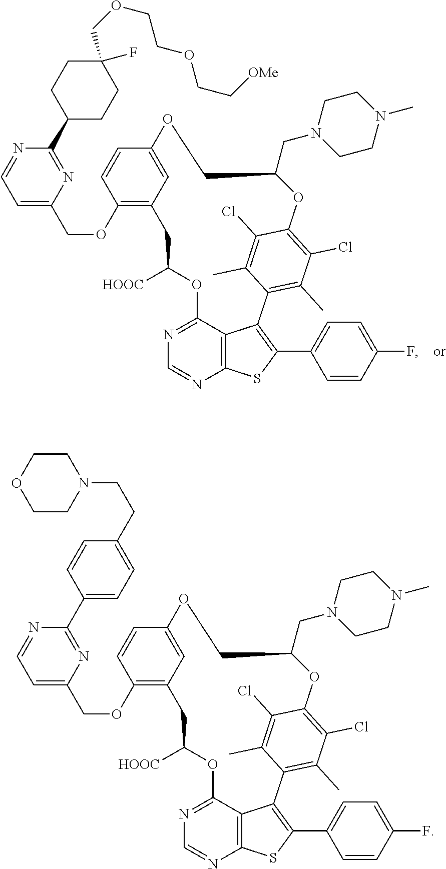 [SMILES: COCCOCCOC[C@]1(F)CC[C@@H](c2nccc(COc3ccc4cc3C[C@H](C(=O)O)Oc3ncnc5sc(-c6ccc(F)cc6)c(c35)-c3c(C)c(Cl)c(c(Cl)c3C)O[C@H](CN3CCN(C)CC3)CO4)n2)CC1 ; Cc1c(Cl)c2c(Cl)c(C)c1-c1c(-c3ccc(F)cc3)sc3ncnc(c13)O[C@@H](C(=O)O)Cc1cc(ccc1OCc1ccnc(-c3ccc(CCN4CCOCC4)cc3)n1)OC[C@@H](CN1CCN(C)CC1)O2]

The BH3 mimetics D¹ and/or D² comprise a formula selected from Table A1 or Ala or an enantiomer, diastereoisomer, atropisomer, deuterated derivative, and/or a pharmaceutically acceptable salt of any of the foregoing.

b. Bcl-xL inhibitors

Suitable BH3 mimetics D¹ and/or D² may comprise a Bcl-xL inhibitor compound of the formulas (II), (IIA), (IIB), (IIC), (III), (IIIA), (IIIB) or (IIIC) or an enantiomer, diastereoisomer, and/or addition salt thereof with a pharmaceutically acceptable acid or base. Additionally, the BH3 mimetics D¹ and/or D² may comprise any compounds of the Bcl-xL inhibitors described herein.

In some embodiments, the BH3 mimetics D¹ and/or D² comprise a formula selected from Table A2 or A2a.

In some embodiments, the BH3 mimetics D¹ and/or D² comprise Bcl-xL inhibitor known in the art, for example, ABT-737 and ABT-263.

In some embodiments, the BH3 mimetics D¹ and/or D² comprise a Bcl-xL inhibitor selected from:

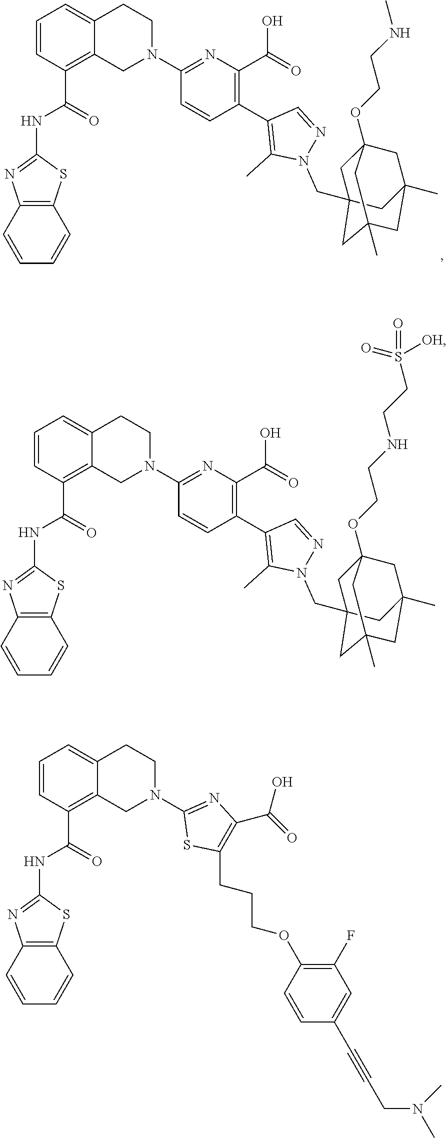 [SMILES: CNCCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2 ; Cc1c(-c2ccc(N3CCc4cccc(C(=O)Nc5nc6ccccc6s5)c4C3)nc2C(=O)O)cnn1CC12CC3(C)CC(C)(C1)CC(OCCNCCS(=O)(=O)O)(C3)C2 ; CN(C)CC#Cc1ccc(OCCCc2sc(N3CCc4cccc(C(=O)Nc5nc6ccccc6s5)c4C3)nc2C(=O)O)c(F)c1]

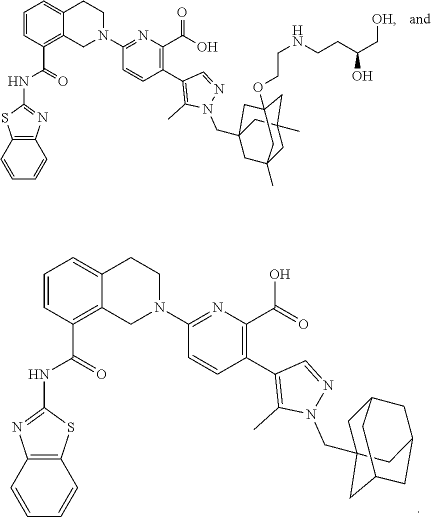 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCNCC[C@H](O)CO)(C3)C2 ; Cc1c(-c2ccc(N3CCc4cccc(C(=O)Nc5nc6ccccc6s5)c4C3)nc2C(=O)O)cnn1CC12CC3CC(CC(C3)C1)C2]

c. Bcl-2 inhibitors

Suitable BH3 mimetics D¹ and/or D² may comprise a Bcl-2 inhibitor compound of the formulas (IV) or (V) or an enantiomer, diastereoisomer, and/or addition salt thereof with a pharmaceutically acceptable acid or base. Additionally, the BH3 mimetics D¹ and/or D² may comprise any compounds of the Bcl-2 inhibitor described herein.

In some embodiments, the Bcl-2 inhibitor is represented by Formula (IV) or an enantiomer, a diastereoisomer, and/or a pharmaceutically acceptable salt of any one of the foregoing.

In some embodiments, in formula (IV), it being understood that:
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen (including quaternary nitrogens),
“cycloalkyl” means any mono- or bi-cyclic, non-aromatic, carbocyclic group containing from 3 to 10 ring members,
“heterocycloalkyl” means any mono- or bi-cyclic, non-aromatic, condensed or spiro group composed of from 3 to 10 ring members and containing from 1 to 3 hetero atoms selected from oxygen, sulphur, SO, SO₂ and nitrogen, it being possible for the aryl, heteroaryl, cycloalkyl and heterocycloalkyl groups so defined and the groups alkyl, alkenyl, alkynyl and alkoxy to be substituted by from 1 to 3 groups selected from linear or branched (C₁-C₆)alkyl, (C₃-C₆)spiro, linear or branched (C₁ C₆)alkoxy, (C₁-C₆)alkyl-S—, hydroxy, oxo (or N-oxide where appropriate), nitro, cyano, —COOR′, —OCOR′, NR′R″, linear or branched (C₁-C₆)polyhaloalkyl, trifluoromethoxy, (C₁ C₆)alkylsulphonyl, halogen, aryl, heteroaryl, aryloxy, arylthio, cycloalkyl, heterocycloalkyl optionally substituted by one or more halogen atoms or alkyl groups.

In some embodiments, in Formula (IV), A₁ represents a hydrogen atom or a methyl group.

In some embodiments, in Formula (IV), A₁ and A₂ both represent a methyl group.

In some embodiments, in Formula (IV), T represents a methyl, aminomethyl, (morpholin-4-yl)methyl, (4-methylpiperazin-1-yl)methyl, 2-(morpholin-4-yl)ethyl, [2-(morpholin-4-yl)ethoxy]methyl, hydroxymethyl, [2-(dimethylamino)ethoxy]methyl, hexahydropyrazino[2,1-c][1,4]oxazin-8(1H)-ylmethyl, 1-oxa-6-azaspiro[3.3]hept-6-ylmethyl, 3-(morpholin-4-yl)propyl or trifluoromethyl group.

In some embodiments, in Formula (IV), R₃ represents a group selected from phenyl, 1H-pyrazole, 1H-indole, 1H-indazole, pyridine, pyrimidine, 1H-pyrrolo[2,3-b]pyridine, 2,3-dihydro-1H-pyrrolo[2,3-b]pyridine, 1H-benzimidazole, 1H-pyrrole, 1H-pyrrolo[2,3-c]pyridine, 1H-pyrrolo[3,2-b]pyridine, 5H-pyrrolo[3,2-d]pyrimidine, thiophene, pyrazine, 1H-pyrazolo[3,4-b]pyridine, 1,2-oxazole, and pyrazolo[1,5-a]pyrimidine, those groups optionally having one or more substituents selected from halogen, linear or branched (C₁-C₆)alkyl, linear or branched (C₁ C₆)alkoxy, cyano, cyclopropyl, oxetane, tetrahydrofuran, —CO—O-CH3, trideuteriomethyl, 2-(morpholin-4-yl)ethyl and 2-(morpholin-4-yl)ethoxy

In some embodiments, the Bcl-2 inhibitor is represented by Formula (V) or (Va) or an enantiomer, a diastereoisomer, and/or a pharmaceutically acceptable salt of any one of the foregoing.

In some embodiments, in Formula (V) or (Va), R₃ represents the following group:

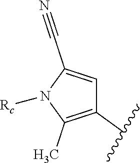

and R_{c} represents a group selected from: hydrogen, linear or branched (C₁-C₆)alkyl group optionally substituted by 1 to 3 halogen atoms, (C₁-C₆)alkylene-NRdRe, (C₁-C₆)alkylene-ORⱼ, cycloalkyl, heterocycloalkyl, and (C₁-C₆)alkylene-heterocycloalkyl group. In some embodiments, Rcrepresents a methyl group..

In some embodiments, in formula (V), R₄ in Formula (V) or (Va) represents the following group:

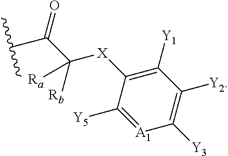

In some embodiments, the Bcl-2 inhibitor is represented by Formula (Vb),. or an enantiomer, a diastereoisomer, and/or a pharmaceutically acceptable salt of any one of the foregoing.

In some embodiments, in Formula (Vb), R_{c} represents a methyl group.

In some embodiments, the Bcl-2 inhibitor is represented by Formula (Vc), (Vd), (Ve), (Vf), (Vg), (Vh), (Vi) or (Vj) or an enantiomer, a diastereoisomer, and/or a pharmaceutically acceptable salt of any one of the foregoing.

In some embodiments, the Bcl-2 inhibitor is represented by Formula (V), (Va), (Vb), (Vc), (Vd), (Ve), (Vf), (Vg), (Vh), (Vi) or (Vj), wherein:
(i) X represents a bond;
(ii) A₁ represents C—Y₄;
(iii) Rₐ and R_{b} represent both a hydrogen atom;
(iv) R₅ represents a hydrogen atom, a hydroxy group or a fluorine atom, preferably a hydroxy group;
(v) R₆ represents a hydrogen atom, a fluorine atom, preferably a hydrogen atom;
(vi) A₁ represents C—H and Y₂ represents a hydrogen atom;
(vii) Yⱼ and Y₅ represent both a hydrogen atom, or: Yⱼ and Y₅ represent a fluoro atom and a hydrogen atom, respectively;
(viii) Y₃ represents a —O—(C₁-C₆)alkylene-heterocycloalkyl group;
(ix) Y₃ represents a group selected from: 2-(morpholin-4-yl)ethoxy, 2-[4-(2,2-difluoroethyl)piperazin-1-yl]ethoxy, 2-(3-fluoroazetidin-1-yl)ethoxy, 2-(3,3-difluoropyrrolidin-1-yl)ethoxy, 2-(oxan-4-yl)ethoxy, 2-(4-fluoropiperidin-1-yl)ethoxy, 2-(thiomorpholin-4-yl)ethoxy, 2-(2-methylmorpholin-4-yl)ethoxy, 2-{6-oxa-9-azaspiro[4.5]decan-9-yl}ethoxy, 2-(3,3-difluoropyrrolidin-1-yl)ethoxy, 2-{4-oxa-7-azaspiro[2.5]octan-7-yl}ethoxy, 2,6-dimethylmorpholin-4-yl]ethoxy, 2-[cyclopropyl(methyl)amino]ethoxy, 2-{methyl[(oxetan-3-yl)methyl]amino}ethoxy, 2-[methyl(oxetan-3-yl)amino]ethoxy, 2-(4-fluoropiperidin-1-yl)ethoxy, 2-[(2-fluoroethyl)(methyl)amino]ethoxy, 2-[4-(2-fluoroethyl)piperazin-1-yl]ethoxy, 2-(4-methylpiperazin-1-yl)ethoxy, 2-(2,2-dimethylmorpholin-4-yl)ethoxy, 2-(morpholin-4-yl)propoxy, 2-(4,4-difluoropiperidin-1-yl)ethyl, [2-methyl-1-(morpholin-4-yl)propan-2-yl]oxy, 2-(3,3-dimethylmorpholin-4-yl)ethoxy, and [(oxan-4-yl)methoxy]methyl;
(x) the group:

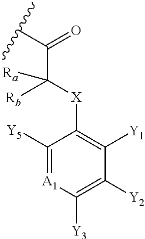

represents

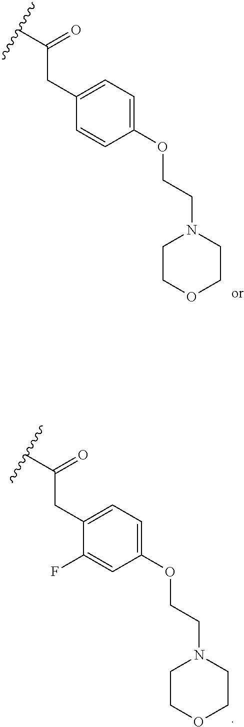 [SMILES: *C(=O)Cc1ccc(OCCN2CCOCC2)cc1 ; *C(=O)Cc1ccc(OCCN2CCOCC2)cc1F]

(xi) T represents a linear or branched (C₁-C₆)alkyl group or a (C₁-C₄)alkylene-NR₁R₂ group; and/or
(xii) T represents a group selected from: methyl, (piperidin-1-yl)methyl, (morpholin-4-yl)methyl, [(3R)-3-fluoropyrrolidin-1-yl]methyl, [methyl(propan-2-yl)amino]methyl, (azepan-1-yl)methyl, (pyrrolidin-1-yl)methyl, [(3S)-3-methylpiperidin-1-yl]methyl, [(3R)-3-methylpiperidin-1-yl]methyl, [(1RS,5SR)-3-azabicyclo[3.1.0]hexan-3-yl]methyl, [(2S)-2-methylpiperidin-1-yl]methyl, {6-azaspiro[2.5]octan-6-yl}methyl, (4,4-difluoropiperidin-1-yl)methyl, (4-methylpiperidin-1-yl)methyl, [ethyl(propan-2-yl)amino]methyl, (3R)-3-methylpyrrolidin-1-yl]methyl, and (3S)-3-{[(3S)-3-methylpyrrolidin-1-yl]methyl. In some embodiments, the linker-drug (or “linker-payload”) moiety -(L-D) may comprise a compounds in Table B or an enantiomer, diastereoisomer, deuterated derivative, and/or a pharmaceutically acceptable salt of any of the foregoing.

In some embodiments, the BH3 mimetics D¹ and/or D² comprises a formula selected from Table A3 or A3a.

d. Topoisomerase 1 Inhibitors

In some embodiments, one of D¹ and D² comprises a topoisomerase 1 inhibitor, or an enantiomer, diastereoisomer, atropisomer, deuterated derivative, and/or addition salt thereof with a pharmaceutically acceptable acid or base.

In some embodiments, D¹ or D² comprises a topoisomerase 1 inhibitor linked to the dual linker, wherein the topoisomerase 1 inhibitor is topotecan, exatecan, deruxtecan or SN-38. In some embodiments, D¹ or D² comprises a formula selected from Table A4 or A4a.

e. Anti-Mitotic Drugs

In some embodiments, one of D¹ and D² comprises an anti-mitotic drug, or an enantiomer, diastereoisomer, atropisomer, deuterated derivative, and/or addition salt thereof with a pharmaceutically acceptable acid or base.

In some embodiments, one of D¹ and D² comprises an anti-mitotic drug linked to the dual linker, wherein the anti-mitotic drug is monomethyl auristatin E (MMAE) or a taxane. In one embodiment, the taxane is selected from docetaxel, paclitaxel, or cabazitaxel.

### Drug Loading

Drug loading is represented by p (or 2a in ADCs of formula (1) of the present disclosure), and is also referred to herein as the drug-to-antibody ratio (DAR). Drug loading may range from 2 to 32 drug moieties per antibody or antigen-binding fragment. In some embodiments, a is an integer from 1 to 16. In some embodiments, a is an integer from 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, a is an integer from 2 to 10, 2 to 9, 2 to 8, 2 to 7, 2 to 6, 2 to 5, 2 to 4, or 2 to 3. In some embodiments, a is an integer from 1 to 16. In some embodiments, a is an integer from 1 to 8. In some embodiments, a is an integer from 1 to 5. In some embodiments, a is an integer from 2 to 4. In some embodiments, a is 1, 2, 3, 4, 5, 6, 7, or 8. In some embodiments, a is 2. In some embodiments, a is 4.

Drug loading may be limited by the number of attachment sites on the antibody or antigen-binding fragment. In some embodiments, the linker moiety (L) of the ADC attaches to the antibody or antigen-binding fragment through a chemically active group on one or more amino acid residues on the antibody or antigen-binding fragment. For example, the linker may be attached to the antibody or antigen-binding fragment via a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, or to the sulfhydryl group of one or more cysteine residues). The site to which the linker is attached can be a natural residue in the amino acid sequence of the antibody or antigen-binding fragment, or it can be introduced into the antibody or antigen-binding fragment, e.g., by DNA recombinant technology (e.g., by introducing a cysteine residue into the amino acid sequence) or by protein biochemistry (e.g., by reduction, pH adjustment, or hydrolysis).

In some embodiments, the number of drug moieties that can be conjugated to an antibody or antigen-binding fragment is limited by the number of free cysteine residues. For example, where the attachment is a cysteine thiol group, an antibody may have only one or a few cysteine thiol groups, or may have only one or a few sufficiently reactive thiol groups through which a linker may be attached. Generally, antibodies do not contain many free and reactive cysteine thiol groups that may be linked to a drug moiety. Indeed, most cysteine thiol residues in antibodies are involved in either interchain or intrachain disulfide bonds. Conjugation to cysteines can therefore, in some embodiments, require at least partial reduction of the antibody. Over-attachment of linker-toxin to an antibody may destabilize the antibody by reducing the cysteine residues available to form disulfide bonds. Therefore, an optimal drug:antibody ratio should increase potency of the ADC (by increasing the number of attached drug moieties per antibody) without destabilizing the antibody or antigen-binding fragment. In some embodiments, an optimal ratio may be 2, 4, 6, or 8. In some embodiments, an optimal ratio may be 2 or 4.

In some embodiments, an antibody or antigen-binding fragment is exposed to reducing conditions prior to conjugation in order to generate one or more free cysteine residues. An antibody, in some embodiments, may be reduced with a reducing agent such as dithiothreitol (DTT) or tris(2-carboxyethyl)phosphine (TCEP), under partial or total reducing conditions, to generate reactive cysteine thiol groups. Unpaired cysteines may be generated through partial reduction with limited molar equivalents of TCEP, which can reduce the interchain disulfide bonds which link the light chain and heavy chain (one pair per H-L pairing) and the two heavy chains in the hinge region (two pairs per H-H pairing in the case of human IgGI) while leaving the intrachain disulfide bonds intact (Stefano et al. (2013) Methods Mol Biol. 1045:145-71). In embodiments, disulfide bonds within the antibodies are reduced electrochemically, e.g., by employing a working electrode that applies an alternating reducing and oxidizing voltage. This approach can allow for on-line coupling of disulfide bond reduction to an analytical device (e.g., an electrochemical detection device, an NMR spectrometer, or a mass spectrometer) or a chemical separation device (e.g., a liquid chromatograph (e.g., an HPLC) or an electrophoresis device (see, e.g., US 2014/0069822)). In some embodiments, an antibody is subjected to denaturing conditions to reveal reactive nucleophilic groups on amino acid residues, such as cysteine.

The drug loading of an ADC may be controlled in different ways, e.g., by: (i) limiting the molar excess of drug-linker intermediate or linker reagent relative to antibody; (ii) limiting the conjugation reaction time or temperature; (iii) partial or limiting reductive conditions for cysteine thiol modification; and/or (iv) engineering by recombinant techniques the amino acid sequence of the antibody such that the number and position of cysteine residues is modified for control of the number and/or position of linker-drug attachments.

In some embodiments, free cysteine residues are introduced into the amino acid sequence of the antibody or antigen-binding fragment. For example, cysteine engineered antibodies can be prepared wherein one or more amino acids of a parent antibody are replaced with a cysteine amino acid. Any form of antibody may be so engineered, i.e. mutated. For example, a parent Fab antibody fragment may be engineered to form a cysteine engineered Fab referred to as a “ThioFab.” Similarly, a parent monoclonal antibody may be engineered to form a “ThioMab.” A single site mutation yields a single engineered cysteine residue in a ThioFab, whereas a single site mutation yields two engineered cysteine residues in a ThioMab, due to the dimeric nature of the IgG antibody. DNA encoding an amino acid sequence variant of the parent polypeptide can be prepared by a variety of methods known in the art (see, e.g., the methods described in WO 2006/034488). These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may also be constructed by restriction fragment manipulation or by overlap extension PCR with synthetic oligonucleotides. ADCs of Formula (1) include, but are not limited to, antibodies that have 1, 2, 3, or 4 engineered cysteine amino acids (Lyon et al. (2012) Methods Enzymol. 502:123-38). In some embodiments, one or more free cysteine residues are already present in an antibody or antigen-binding fragment, without the use of engineering, in which case the existing free cysteine residues may be used to conjugate the antibody or antigen-binding fragment to a drug moiety.

Where more than one nucleophilic group reacts with a drug-linker intermediate or a linker moiety reagent followed by drug moiety reagent, in a reaction mixture comprising multiple copies of the antibody or antigen-binding fragment and linker moiety, then the resulting product can be a mixture of ADC compounds with a distribution of one or more drug moieties attached to each copy of the antibody or antigen-binding fragment in the mixture. In some embodiments, the drug loading in a mixture of ADCs resulting from a conjugation reaction ranges from 1 to 16 drug moieties attached per antibody or antigen-binding fragment. The average number of drug moieties per antibody or antigen-binding fragment (i.e., the average drug loading, or average p) may be calculated by any conventional method known in the art, e.g., by mass spectrometry (e.g., liquid chromatography-mass spectrometry (LC-MS)) and/or high-performance liquid chromatography (e.g., HIC-HPLC). In some embodiments, the average number of drug moieties per antibody or antigen-binding fragment is determined by liquid chromatography-mass spectrometry (LC-MS). In some embodiments, the average number of drug moieties per antibody or antigen-binding fragment is from about 1.5 to about 3.5, about 2.5 to about 4.5, about 3.5 to about 5.5, about 4.5 to about 6.5, about 5.5 to about 7.5, about 6.5 to about 8.5, or about 7.5 to about 9.5. In some embodiments, the average number of drug moieties per antibody or antigen-binding fragment is from about 2 to about 4, about 3 to about 5, about 4 to about 6, about 5 to about 7, about 6 to about 8, about 7 to about 9, about 2 to about 8, or about 4 to about 8.

In some embodiments, the average number of drug moieties per antibody or antigen-binding fragment is about 2. In some embodiments, the average number of drug moieties per antibody or antigen-binding fragment is about 1.5, about 1.6, about 1.7, about 1.8, about 1.9, about 2, about 2.1, about 2.2, about 2.3, about 2.4, or about 2.5. In some embodiments, the average number of drug moieties per antibody or antigen-binding fragment is 2.

In some embodiments, the average number of drug moieties per antibody or antigen-binding fragment is about 4. In some embodiments, the average number of drug moieties per antibody or antigen-binding fragment is about 3.5, about 3.6, about 3.7, about 3.8, about 3.9, about 4, about 4.1, about 4.2, about 4.3, about 4.4, or about 4.5. In some embodiments, the average number of drug moieties per antibody or antigen-binding fragment is 4.

In some embodiments, the term “about,” as used with respect to the average number of drug moieties per antibody or antigen-binding fragment, means plus or minus 20%, 15%, 10%, 5%, or 1%. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Individual ADC compounds, or “species,” may be identified in the mixture by mass spectroscopy and separated by, e.g., UPLC or HPLC, e.g. hydrophobic interaction chromatography (HIC-HPLC). In some embodiments, a homogeneous or nearly homogenous ADC product with a single loading value may be isolated from the conjugation mixture, e.g., by electrophoresis or chromatography.

In some embodiments, higher drug loading (e.g., p>16) may cause aggregation, insolubility, toxicity, or loss of cellular permeability of certain antibody-drug conjugates. Higher drug loading may also negatively affect the pharmacokinetics (e.g., clearance) of certain ADCs. In some embodiments, lower drug loading (e.g., p<2) may reduce the potency of certain ADCs against target-expressing cells. In some embodiments, the drug loading for an ADC of the present disclosure ranges from about 2 to about 16, about 2 to about 10, about 2 to about 8; from about 2 to about 6; from about 2 to about 5; from about 3 to about 5; from about 2 to about 4; or from about 4 to about 8.

In some embodiments, a drug loading and/or an average drug loading of about 2 is achieved, e.g., using partial reduction of intrachain disulfides on the antibody or antigen-binding fragment, and provides beneficial properties. In some embodiments, a drug loading and/or an average drug loading of about 4 or about 6 or about 8 is achieved, e.g., using partial reduction of intrachain disulfides on the antibody or antigen-binding fragment, and provides beneficial properties. In some embodiments, a drug loading and/or an average drug loading of less than about 2 may result in an unacceptably high level of unconjugated antibody species, which can compete with the ADC for binding to a target antigen and/or provide for reduced treatment efficacy. In some embodiments, a drug loading and/or average drug loading of more than about 16 may result in an unacceptably high level of product heterogeneity and/or ADC aggregation. A drug loading and/or an average drug loading of more than about 16 may also affect stability of the ADC, due to loss of one or more chemical bonds required to stabilize the antibody or antigen-binding fragment.

The present disclosure includes methods of producing the described ADCs. Briefly, the ADCs comprise an antibody or antigen-binding fragment as the antibody or antigen-binding fragment, a drug moiety (e.g., a Bcl-xL inhibitor), and a linker that joins the drug moiety and the antibody or antigen-binding fragment. In some embodiments, the ADCs can be prepared using a linker having reactive functionalities for covalently attaching to the drug moiety and to the antibody or antigen-binding fragment. In some embodiments, the antibody or antigen-binding fragment is functionalized to prepare a functional group that is reactive with a linker or a drug-linker intermediate. For example, in some embodiments, a cysteine thiol of an antibody or antigen-binding fragment can form a bond with a reactive functional group of a linker or a drug-linker intermediate to make an ADC. In some embodiments, an antibody or antigen-binding fragment is prepared with bacterial transglutaminase (BTG) - reactive glutamines specifically functionalized with an amine containing cyclooctyne BCN (N-[(1R,8S,9s)-Bicyclo[6.1.0]non-4-yn-9-ylmethyloxycarbonyl]-1,8-diamino-3,6-dioxaoctane) moiety. In some embodiments, site-specific conjugation of a linker or a drug-linker intermediate to a BCN moiety of an antibody or antigen-binding fragment is performed, e.g., as described and exemplified herein. The generation of the ADCs can be accomplished by techniques known to the skilled artisan.

In some embodiments, an ADC is produced by contacting an antibody or antigen-binding fragment with a linker and a drug moiety (e.g., a Bcl-xL inhibitor) in a sequential manner, such that the antibody or antigen-binding fragment is covalently linked to the linker first, and then the pre-formed antibody-linker intermediate reacts with the drug moiety. The antibody-linker intermediate may or may not be subjected to a purification step prior to contacting the drug moiety. In other embodiments, an ADC is produced by contacting an antibody or antigen-binding fragment with a linker-drug compound pre-formed by reacting a linker with a drug moiety. The pre-formed linker-drug compound may or may not be subjected to a purification step prior to contacting the antibody or antigen-binding fragment. In other embodiments, the antibody or antigen-binding fragment contacts the linker and the drug moiety in one reaction mixture, allowing simultaneous formation of the covalent bonds between the antibody or antigen-binding fragment and the linker, and between the linker and the drug moiety. This method of producing ADCs may include a reaction, wherein the antibody or antigen-binding fragment contacts the antibody or antigen-binding fragment prior to the addition of the linker to the reaction mixture, and vice versa. In some embodiments, an ADC is produced by reacting an antibody or antigen-binding fragment with a linker joined to a drug moiety, such as a Bcl-xL inhibitor, under conditions that allow conjugation.

The ADCs prepared according to the methods described above may be subjected to a purification step. The purification step may involve any biochemical methods known in the art for purifying proteins, or any combination of methods thereof. These include, but are not limited to, tangential flow filtration (TFF), affinity chromatography, ion exchange chromatography, any charge or isoelectric point-based chromatography, mixed mode chromatography, e.g., CHT (ceramic hydroxyapatite), hydrophobic interaction chromatography, size exclusion chromatography, dialysis, filtration, selective precipitation, or any combination thereof.

### 2. Therapeutic Uses and Compositions

Disclosed herein are methods of using the compositions described herein, e.g., the disclosed ADC compounds and compositions, in treating a subject for a disorder, e.g., a cancer. Compositions, e.g., ADCs, may be administered alone or in combination with at least one additional inactive and/or active agent, e.g., at least one additional therapeutic agent, and may be administered in any pharmaceutically acceptable formulation, dosage, and dosing regimen. Treatment efficacy may be evaluated for toxicity as well as indicators of efficacy and adjusted accordingly. Efficacy measures include, but are not limited to, a cytostatic and/or cytotoxic effect observed in vitro or in vivo, reduced tumor volume, tumor growth inhibition, and/or prolonged survival.

Methods of determining whether an ADC exerts a cytostatic and/or cytotoxic effect on a cell are known. For example, the cytotoxic or cytostatic activity of an ADC can be measured by, e.g., exposing mammalian cells expressing a target antigen of the ADC in a cell culture medium; culturing the cells for a period from about 6 hours to about 6 days; and measuring cell viability (e.g., using a CellTiter-Glo® (CTG) or MTT cell viability assay). Cell-based in vitro assays may also be used to measure viability (proliferation), cytotoxicity, and induction of apoptosis (caspase activation) of the ADC.

For determining cytotoxicity, necrosis or apoptosis (programmed cell death) may be measured. Necrosis is typically accompanied by increased permeability of the plasma membrane, swelling of the cell, and rupture of the plasma membrane. Apoptosis can be quantitated, for example, by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica (1999) 2:34-7 (Roche Molecular Biochemicals).

Apoptosis may also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al., eds. (1992) pp. 3.17.1-3.17.16). Cells also can be labeled with a DNA dye (e.g., acridine orange, ethidium bromide, or propidium iodide) and the cells observed for chromatin condensation and margination along the inner nuclear membrane. Apoptosis may also be determined, in some embodiments, by screening for caspase activity. In some embodiments, a Caspase-Glo® Assay can be used to measure activity of caspase-3 and caspase-7. In some embodiments, the assay provides a luminogenic caspase-3/7 substrate in a reagent optimized for caspase activity, luciferase activity, and cell lysis. In some embodiments, adding Caspase-Glo® 3/7 Reagent in an “add-mix-measure” format may result in cell lysis, followed by caspase cleavage of the substrate and generation of a “glow-type” luminescent signal, produced by luciferase. In some embodiments, luminescence may be proportional to the amount of caspase activity present, and can serve as an indicator of apoptosis. Other morphological changes that can be measured to determine apoptosis include, e.g., cytoplasmic condensation, increased membrane blebbing, and cellular shrinkage. Determination of any of these effects on cancer cells indicates that an ADC is useful in the treatment of cancers.

Cell viability may be measured, e.g., by determining in a cell the uptake of a dye such as neutral red, trypan blue, Crystal Violet, or ALAMAR™ blue (see, e.g., Page et al. (1993) Intl J Oncology 3:473-6). In such an assay, the cells are incubated in media containing the dye, the cells are washed, and the remaining dye, reflecting cellular uptake of the dye, is measured spectrophotometrically.

Cell viability may also be measured, e.g., by quantifying ATP, an indicator of metabolically active cells. In some embodiments, in vitro potency and/or cell viability of prepared ADCs or antineoplastic payloads, such as BH3 mimetic compounds (e.g., MCl-1 inhibitor, Bcl-xL inhibitor or Bel-2 inhibitor) or topoisomerase 1 inhibitors (e.g., topotecan, exatecan, deruxtecan or SN-38) or anti-mitotic drugs (e.g., monomethyl auristatin E (MMAE) or a taxane) may be assessed using a CellTiter-Glo® (CTG) cell viability assay, as described in the examples provided herein. In this assay, in some embodiments, the single reagent (CellTiter-Glo® Reagent) is added directly to cells cultured in serum-supplemented medium. The addition of reagent results in cell lysis and generation of a luminescent signal proportional to the amount of ATP present. The amount of ATP is directly proportional to the number of cells present in culture.

Cell viability may also be measured, e.g., by measuring the reduction of tetrazolium salts. In some embodiments, in vitro potency and/or cell viability of prepared ADCs or antineoplastic payloads, such as BH3 mimetic compounds (e.g., MCl-1 inhibitor, Bcl-xL inhibitor or Bcl-2 inhibitor) or topoisomerase 1 inhibitors (e.g., topotecan, exatecan, deruxtecan or SN-38) or anti-mitotic drugs (e.g., monomethyl auristatin E (MMAE) or a taxane) may be assessed using an MTT cell viability assay, as described in the examples provided herein. In this assay, in some embodiments, the yellow tetrazolium MTT (3-(4, 5-dimethylthiazolyl-2)-2,5-diphenyltetrazolium bromide) is reduced by metabolically active cells, in part by the action of dehydrogenase enzymes, to generate reducing equivalents such as NADH and NADPH. The resulting intracellular purple formazan can then be solubilized and quantified by spectrophotometric means.

In certain aspects, the present disclosure features a method of killing, inhibiting or modulating the growth of a cancer cell or tissue by disrupting the expression and/or activity of Bcl-2 family protein (e.g., Mcl-1, Bcl-2 and/or Bcl-xL) and/or one or more upstream modulators or downstream targets thereof. The method may be used with any subject where disruption of Bcl-2 family protein expression and/or activity provides a therapeutic benefit. Subjects that may benefit from disrupting Bcl-2 family protein expression and/or activity include, but are not limited to, those having or at risk of having a cancer such as a tumor or a hematological cancer. In some embodiments, the cancer is a breast cancer, multiple myeloma, plasma cell myeloma, leukemia, lymphoma, sarcoma, gastric cancer, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

In some embodiments, the disclosed ADCs may be administered in any cell or tissue that expresses PCAD, such as a PCAD-expressing cancer cell or tissue. An exemplary embodiment includes a method of killing a PCAD-expressing cancer cell or tissue. The method may be used with any cell or tissue that expresses PCAD, such as a cancerous cell or a metastatic lesion. Non-limiting examples of PCAD-expressing cancers include breast cancer, gastric cancer, endometrial cancer, ovarian cancer, pancreatic cancer, bladder cancer, prostate cancer, and melanoma (Vieira and Paredes (2015) Mol Cancer 14:178).

In some embodiments, the disclosed ADCs may be administered in any cell or tissue that expresses CD48, such as a CD48-expressing cancer cell or tissue. An exemplary embodiment includes a method of killing a CD48-expressing cancer cell or tissue. The method may be used with any cell or tissue that expresses CD48, such as a cancerous cell or a metastatic lesion. Non-limiting examples of CD48-expressing cancers include a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer.

In some embodiments, the disclosed ADCs may be administered in any cell or tissue that expresses CD74, such as a CD74-expressing cancer cell or tissue. An exemplary embodiment includes a method of killing a CD74-expressing cancer cell or tissue. The method may be used with any cell or tissue that expresses CD74, such as a cancerous cell or a metastatic lesion. Non-limiting examples of CD74-expressing cancers include a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer.

In some embodiments, the disclosed ADCs may be administered in any cell or tissue that expresses HER2, such as a HER2-expressing cancer cell or tissue. An exemplary embodiment includes a method of killing a HER2-expressing cancer cell or tissue. The method may be used with any cell or tissue that expresses HER2, such as a cancerous cell or a metastatic lesion. Non-limiting examples of HER2-expressing cancers include breast cancer, gastric cancer, bladder cancer, urothelial cell carcinoma, esophageal cancer, lung cancer (e.g., lung adenocarcinoma), uterine cancer (e.g., uterine serous endometrial carcinoma), salivary duct carcinoma, cervical cancer, endometrial cancer, and ovarian cancer (English et al. (2013) Mol Diagn Ther. 17:85-99). Non-limiting examples of HER2-expressing cells include HCC1954 and HCC2218 breast cancer cells, and cells comprising a recombinant nucleic acid encoding HER2 or a portion thereof.

In some embodiments, the disclosed ADCs may be administered in any cell or tissue that expresses EphA2, such as a EphA2-expressing cancer cell or tissue. An exemplary embodiment includes a method of killing a EphA2-expressing cancer cell or tissue. The method may be used with any cell or tissue that expresses EphA2, such as a cancerous cell or a metastatic lesion. Non-limiting examples of EphA2-expressing cancers include breast cancer, non-small cell lung cancer, pancreatic, esophageal, head and neck, stomach, bladder, and colon cancers. In some embodiments, EphA2-expressing cancer is breast cancer or non-small cell lung cancer.

In some embodiments, the disclosed ADCs may be administered in any cell or tissue that expresses TROP2, such as a TROP2-expressing cancer cell or tissue. An exemplary embodiment includes a method of killing a TROP2-expressing cancer cell or tissue. The method may be used with any cell or tissue that expresses TROP2, such as a cancerous cell or a metastatic lesion. Non-limiting examples of TROP2-expressing cancers include breast cancer, non-small cell lung cancer, pancreatic, esophageal, head and neck, stomach, bladder, and colon cancers. In some embodiments, TROP2-expressing cancer is breast cancer or non-small cell lung cancer.

In some embodiments, the disclosed ADCs may be administered in any cell or tissue that expresses B7-H3, such as a B7-H3-expressing cancer cell or tissue. An exemplary embodiment includes a method of killing a B7-H3-expressing cancer cell or tissue. The method may be used with any cell or tissue that expresses B7-H3, such as a cancerous cell or a metastatic lesion. Non-limiting examples of B7-H3-expressing cancers include breast cancer, non-small cell lung cancer, pancreatic, esophageal, head and neck, stomach, bladder, and colon cancers. In some embodiments, B7-H3-expressing cancer is breast cancer or non-small cell lung cancer.

In some embodiments, the disclosed ADCs may be administered in any cell or tissue that expresses 5T4, such as a 5T4-expressing cancer cell or tissue. An exemplary embodiment includes a method of killing a 5T4-expressing cancer cell or tissue. The method may be used with any cell or tissue that expresses 5T4, such as a cancerous cell or a metastatic lesion. Non-limiting examples of 5T4-expressing cancers include breast cancer, non-small cell lung cancer, pancreatic, esophageal, head and neck, stomach, bladder, and colon cancers. In some embodiments, 5T4-expressing cancer is breast cancer or non-small cell lung cancer.

In some embodiments, the disclosed ADCs may be administered in any cell or tissue that expresses MET, such as a MET-expressing cancer cell or tissue. An exemplary embodiment includes a method of killing a MET-expressing cancer cell or tissue. The method may be used with any cell or tissue that expresses MET, such as a cancerous cell or a metastatic lesion. Non-limiting examples of MET-expressing cancers include a melanoma, uveal melanoma, renal cancer including papillary renal cell carcinoma, thyroid cancer, mesothelioma, liver hepatocellular cancer, lung cancer including non-small cell lung cancer and small cell lung cancer, gastric cancer including stomach cancer, pancreatic cancer, colorectal cancer, esophageal cancer, cholangiocarcinoma, head and neck cancer including oral cancer, cervical and endocervical cancer, bladder and urothelial cancer, uterine cancer, ovarian cancer, breast cancer, prostate cancer, sarcoma, testicular cancer, glioblastoma, adrenocortical cancer, brain cancer, spleen cancer, thymoma, multiple myeloma, plasma cell myeloma, leukemia, lymphoma, acute myeloid leukemia, bone marrow cancer, chronic lymphocytic leukemia, lymphoblastic leukemia including acute lymphoblastic leukemia, follicular lymphoma, lymphoid malignancies of T-cell or B-cell origin, myelogenous leukemia, or myeloma. Non-limiting examples of MET-expressing cells include the cancer cell population from a melanoma, uveal melanoma, renal cancer, thyroid cancer, mesothelioma, liver hepatocellular cancer, lung cancer including non-small cell lung cancer and small cell lung cancer, gastric cancer including stomach cancer, pancreatic cancer, colorectal cancer, esophageal cancer, cholangiocarcinoma, head and neck cancer including oral cancer, cervical and endocervical cancer, bladder and urothelial cancer, uterine cancer, ovarian cancer, breast cancer, prostate cancer, sarcoma, testicular cancer, glioblastoma, adrenocortical cancer, brain cancer, spleen cancer, thymoma, multiple myeloma, plasma cell myeloma, leukemia, lymphoma, acute myeloid leukemia, bone marrow cancer, chronic lymphocytic leukemia, lymphoblastic leukemia including acute lymphoblastic leukemia, follicular lymphoma, lymphoid malignancies of T-cell or B-cell origin, myelogenous leukemia, or myeloma, and cells comprising a recombinant nucleic acid encoding MET or a portion thereof.

Exemplary methods include the steps of contacting a cell with an ADC, as described herein, in an effective amount, i.e., an amount sufficient to kill the cell. The method can be used on cells in culture, e.g., in vitro, in vivo, ex vivo, or in situ. For example, cells that express HER2 (e.g., cells collected by biopsy of a tumor or metastatic lesion; cells from an established cancer cell line; or recombinant cells), can be cultured in vitro in culture medium and the contacting step can be affected by adding the ADC to the culture medium. The method will result in killing of cells expressing HER2, including in particular cancer cells expressing HER2. Alternatively, the ADC can be administered to a subject by any suitable administration route (e.g., intravenous, subcutaneous, or direct contact with a tumor tissue) to have an effect in vivo. This approach can be used for antibodies targeting other cell surface antigens (e.g., EGFR, CD7, HER2, CD48, CD74, EphA2, TROP2, B7-H3, 5T4 or MET).

The in vivo effect of a disclosed ADC therapeutic composition can be evaluated in a suitable animal model. For example, xenogeneic cancer models can be used, wherein cancer explants or passaged xenograft tissues are introduced into immune compromised animals, such as nude or SCID mice (Klein et al. (1997) Nature Med. 3:402-8). Efficacy may be predicted using assays that measure inhibition of tumor formation, tumor regression or metastasis, and the like.

In vivo assays that evaluate the promotion of tumor death by mechanisms such as apoptosis may also be used. In some embodiments, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

Further provided herein are methods of treating a disorder, e.g., a cancer. The compositions described herein, e.g., the ADCs disclosed herein, can be administered to a non-human mammal or human subject for therapeutic purposes. The therapeutic methods include administering to a subject having or suspected of having a cancer a therapeutically effective amount of a composition comprising an Bcl-xL inhibitor, e.g., an ADC where the inhibitor is linked to a targeting antibody that binds to an antigen (1) expressed on a cancer cell, (2) is accessible to binding, and/or (3) is localized or predominantly expressed on a cancer cell surface as compared to a non-cancer cell.

An exemplary embodiment is a method of treating a subject having or suspected of having a cancer, comprising administering to the subject a therapeutically effective amount of a composition disclosed herein, e.g., an ADC, composition, or pharmaceutical composition (e.g., any of the exemplary ADCs, compositions, or pharmaceutical compositions disclosed herein). In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74, or EphA2. In some embodiments, the target antigen is CD74, CD48, HER2, TROP2, B7-H3, or 5T4. In some embodiments, the target antigen is CD48, CD74, EphA2, or MET. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a method of delivering antineoplastic payloads (e.g. BH3 mimetics, topoisomerase 1 inhibitors, or anti-mitotic drugs) to a cell expressing PCAD, comprising conjugating the antineoplastic payloads (e.g. BH3 mimetics, topoisomerase 1 inhibitors, or anti-mitotic drugs) to an antibody that immunospecifically binds to a PCAD epitope and exposing the cell to the ADC. Exemplary cancer cells that express PCAD for which the ADCs of the present disclosure are indicated include breast cancer and gastric cancer cells.

Another exemplary embodiment is a method of delivering antineoplastic payloads (e.g. BH3 mimetics, topoisomerase 1 inhibitors, or anti-mitotic drugs) to a cell expressing HER2, comprising conjugating the antineoplastic payloads (e.g. BH3 mimetics, topoisomerase 1 inhibitors, or anti-mitotic drugs) to an antibody that immunospecifically binds to a HER2 epitope and exposing the cell to the ADC. Exemplary cancer cells that express HER2 for which the ADCs of the present disclosure are indicated include breast cancer cells.

Another exemplary embodiment is a method of delivering antineoplastic payloads (e.g. BH3 mimetics, topoisomerase 1 inhibitors, or anti-mitotic drugs) to a cell expressing CD48, comprising conjugating the antineoplastic payloads (e.g. BH3 mimetics, topoisomerase 1 inhibitors, or anti-mitotic drugs) to an antibody that immunospecifically binds to a CD48 epitope and exposing the cell to the ADC. Exemplary cancer cells that express CD48 for which the ADCs of the present disclosure are indicated include hematological cancer cells.

Another exemplary embodiment is a method of delivering antineoplastic payloads (e.g. BH3 mimetics, topoisomerase 1 inhibitors, or anti-mitotic drugs) to a cell expressing CD74, comprising conjugating the antineoplastic payloads (e.g. BH3 mimetics, topoisomerase 1 inhibitors, or anti-mitotic drugs) to an antibody that immunospecifically binds to a CD74 epitope and exposing the cell to the ADC. Exemplary cancer cells that express CD74 for which the ADCs of the present disclosure are indicated include hematological cancer cells.

Another exemplary embodiment is a method of delivering antineoplastic payloads (e.g. BH3 mimetics, topoisomerase 1 inhibitors, or anti-mitotic drugs) to a cell expressing EphA2, comprising conjugating the antineoplastic payloads (e.g. BH3 mimetics, topoisomerase 1 inhibitors, or anti-mitotic drugs) to an antibody that immunospecifically binds to a EphA2 epitope and exposing the cell to the ADC. Exemplary cancer cells that express EphA2 for which the ADCs of the present disclosure are indicated include breast cancer or non-small cell lung cancer.

Another exemplary embodiment is a method of delivering antineoplastic payloads (e.g. BH3 mimetics, topoisomerase 1 inhibitors, or anti-mitotic drugs) to a cell expressing TROP2, comprising conjugating the antineoplastic payloads (e.g. BH3 mimetics, topoisomerase 1 inhibitors, or anti-mitotic drugs) to an antibody that immunospecifically binds to a TROP2 epitope and exposing the cell to the ADC. Exemplary cancer cells that express TROP2 for which the ADCs of the present disclosure are indicated include breast cancer or non-small cell lung cancer.

Another exemplary embodiment is a method of delivering antineoplastic payloads (e.g. BH3 mimetics, topoisomerase 1 inhibitors, or anti-mitotic drugs) to a cell expressing B7-H3, comprising conjugating the antineoplastic payloads (e.g. BH3 mimetics, topoisomerase 1 inhibitors, or anti-mitotic drugs) to an antibody that immunospecifically binds to a B7-H3 epitope and exposing the cell to the ADC. Exemplary cancer cells that express B7-H3 for which the ADCs of the present disclosure are indicated include breast cancer or non-small cell lung cancer.

Another exemplary embodiment is a method of delivering antineoplastic payloads (e.g. BH3 mimetics, topoisomerase 1 inhibitors, or anti-mitotic drugs) to a cell expressing 5T4, comprising conjugating the antineoplastic payloads (e.g. BH3 mimetics, topoisomerase 1 inhibitors, or anti-mitotic drugs) to an antibody that immunospecifically binds to a 5T4 epitope and exposing the cell to the ADC. Exemplary cancer cells that express 5T4 for which the ADCs of the present disclosure are indicated include breast cancer or non-small cell lung cancer.

Another exemplary embodiment is a method of delivering antineoplastic payloads (e.g. BH3 mimetics, topoisomerase 1 inhibitors, or anti-mitotic drugs) to a cell expressing MET, comprising conjugating the antineoplastic payloads (e.g. BH3 mimetics, topoisomerase 1 inhibitors, or anti-mitotic drugs) to an antibody that immunospecifically binds to a MET epitope and exposing the cell to the ADC. Exemplary cancer cells that express MET for which the ADCs of the present disclosure are indicated include pancreatic cancer, renal cancer, liver cancer gastric cancer or lung cancer.

In certain aspects, the present disclosure further provides methods of reducing or inhibiting growth of a tumor (e.g., a CD48-expressing tumor, a CD74-expressing tumor, a PCAD-expressing tumor, an HER2-expressing tumor, a TROP2-expressing tumor, a B7-H3-expressing tumor, a 5T3-expressing tumor, a MET-expressing tumor), comprising administering a therapeutically effective amount of an ADC or composition comprising an ADC. In some embodiments, the treatment is sufficient to reduce or inhibit the growth of the patient's tumor, reduce the number or size of metastatic lesions, reduce tumor load, reduce primary tumor load, reduce invasiveness, prolong survival time, and/or maintain or improve the quality of life. In some embodiments, the tumor is resistant or refractory to treatment with the antibody or antigen-binding fragment of the ADC (e.g., anti-CD48 antibody, an anti-CD74 antibody, an anti-PCAD antibody, an anti-HER2 antibody, an anti-EphA2 antibody, an anti-TROP2-antibody, an anti-B7-H3-antibody, an anti-5T3-antibody, an anti-MET-antibody) when administered alone, and/or the tumor is resistant or refractory to treatment with the Bcl-xL inhibitor drug moiety when administered alone.

An exemplary embodiment is a method of reducing or inhibiting the growth of a tumor in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the tumor expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is CD48, CD74, EphA2, PCAD, HER2, TROP2, B7-H3, or 5T4. In some embodiments, the target antigen is MET. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, or spleen cancer. In some embodiments, the tumor is a gastric cancer. In some embodiments, administration of the ADC, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

Another exemplary embodiment is a method of delaying or slowing the growth of a tumor in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the tumor expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is CD48, CD74, EphA2, PCAD, HER2, TROP2, B7-H3, or 5T4. In some embodiments, the target antigen is CD48, CD74, EphA2, or MET.

In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, or spleen cancer. In some embodiments, the tumor is a gastric cancer. In some embodiments, administration of the ADC, composition, or pharmaceutical composition delays or slows the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

In certain aspects, the present disclosure further provides methods of reducing or slowing the expansion of a cancer cell population (e.g., a CD48-expressing tumor, a CD74-expressing tumor, a PCAD-expressing cancer cell population, a HER2-expressing cancer cell population), comprising administering a therapeutically effective amount of an ADC or composition comprising an ADC.

An exemplary embodiment is a method of reducing or slowing the expansion of a cancer cell population in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the cancer cell population expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8,CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C₅, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HERI, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-R1, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is CD48, CD74, EphA2, PCAD, or HER2, TROP2, B7-H3, or 5T4. In some embodiments, the target antigen is CD48, CD74, EphA2, or MET. In some embodiments, the cancer cell population is from a tumor or a hematological cancer. In some embodiments, the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, or head and neck cancer. In some embodiments, the cancer cell population is from a lymphoma or gastric cancer. In some embodiments, administration of the ADC, composition, or pharmaceutical composition reduces the cancer cell population by at least about 10%, at least about 20%, at least about 30%, at least about 40%, at least about 50%, at least about 60%, at least about 70%, at least about 80%, at least about 90%, at least about 95%, or at least about 99%, as compared to the population in the absence of treatment. In some embodiments, administration of the ADC, composition, or pharmaceutical composition slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%, as compared to expansion in the absence of treatment.

Also provided herein are methods of determining whether a subject having or suspected of having a cancer will be responsive to treatment with the disclosed ADCs and compositions. An exemplary embodiment is a method of determining whether a subject having or suspected of having a cancer will be responsive to treatment with an ADC, composition, or pharmaceutical composition (e.g., any of the exemplary ADCs, compositions, or pharmaceutical compositions disclosed herein) by providing a biological sample from the subject; contacting the sample with the ADC; and detecting binding of the ADC to cancer cells in the sample. In some embodiments, the sample is a tissue biopsy sample, a blood sample, or a bone marrow sample. In some embodiments, the method comprises providing a biological sample from the subject; contacting the sample with the ADC; and detecting one or more markers of cancer cell death in the sample (e.g., increased expression of one or more apoptotic markers, reduced expansion of a cancer cell population in culture, etc.).

Further provided herein are therapeutic uses of the disclosed ADCs and compositions. An exemplary embodiment is an ADC, composition, or pharmaceutical composition (e.g., any of the exemplary ADCs, compositions, or pharmaceutical compositions disclosed herein) for use in treating a subject having or suspected of having a cancer (e.g., a BCMA-expressing cancer, a CD33-expressing cancer, a PCAD-expressing cancer, a HER2-expressing cancer, a EphA2-expressing cancer, a CD48-expressing cancer, a CD74-expressing cancer). Another exemplary embodiment is a use of an ADC, composition, or pharmaceutical composition (e.g., any of the exemplary ADCs, compositions, or pharmaceutical compositions disclosed herein) in treating a subject having or suspected of having a cancer (e.g., a BCMA-expressing cancer, a CD33-expressing cancer, a PCAD-expressing cancer, a HER2-expressing cancer, a EphA2-expressing cancer, a CD48-expressing cancer, a CD74-expressing cancer). Another exemplary embodiment is a use of an ADC, composition, or pharmaceutical composition (e.g., any of the exemplary ADCs, compositions, or pharmaceutical compositions disclosed herein) in a method of manufacturing a medicament for treating a subject having or suspected of having a cancer (e.g., a BCMA-expressing cancer, a CD33-expressing cancer, a PCAD-expressing cancer, a HER2-expressing cancer, a EphA2-expressing cancer, a CD48-expressing cancer, a CD74-expressing cancer). Methods for identifying subjects having cancers that express a target antigen (e.g., CD48, CD74, EphA2, PCAD, HER2, TROP2, B7-H3, or 5T4) are known in the art and may be used to identify suitable patients for treatment with a disclosed ADC compound or composition.

Moreover, ADCs of the present disclosure may be administered to a non-human mammal expressing an antigen with which the ADC is capable of binding for veterinary purposes or as an animal model of human disease. Regarding the latter, such animal models may be useful for evaluating the therapeutic efficacy of the disclosed ADCs (e.g., testing of dosages and time courses of administration).

The therapeutic compositions used in the practice of the foregoing methods may be formulated into pharmaceutical compositions comprising a pharmaceutically acceptable carrier suitable for the desired delivery method. An exemplary embodiment is a pharmaceutical composition comprising an ADC of the present disclosure and a pharmaceutically acceptable carrier, e.g., one suitable for a chosen means of administration, e.g., intravenous administration. The pharmaceutical composition may also comprise one or more additional inactive and/or therapeutic agents that are suitable for treating or preventing, for example, a cancer (e.g., a standard-of-care agent, etc.). The pharmaceutical composition may also comprise one or more carrier, excipient, and/or stabilizer components, and the like. Methods of formulating such pharmaceutical compositions and suitable formulations are known in the art (see, e.g., “Remington's Pharmaceutical Sciences,” Mack Publishing Co., Easton, PA).

Suitable carriers include any material that, when combined with the therapeutic composition, retains the anti-tumor function of the therapeutic composition and is generally non-reactive with the patient's immune system. Pharmaceutically acceptable carriers include any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, mesylate salt, and the like, as well as combinations thereof. In many cases, isotonic agents are included, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the ADC.

A pharmaceutical composition of the present disclosure can be administered by a variety of methods known in the art. The route and/or mode of administration may vary depending upon the desired results. In some embodiments, the therapeutic formulation is solubilized and administered via any route capable of delivering the therapeutic composition to the cancer site. Potentially effective routes of administration include, but are not limited to, parenteral (e.g., intravenous, subcutaneous), intraperitoneal, intramuscular, intratumor, intradermal, intraorgan, orthotopic, and the like. In some embodiments, the administration is intravenous, subcutaneous, intraperitoneal, or intramuscular. The pharmaceutically acceptable carrier should be suitable for the route of administration, e.g., intravenous or subcutaneous administration (e.g., by injection or infusion). Depending on the route of administration, the active compound(s), i.e., the ADC and/or any additional therapeutic agent, may be coated in a material to protect the compound(s) from the action of acids and other natural conditions that may inactivate the compound(s). Administration can be either systemic or local.

The therapeutic compositions disclosed herein may be sterile and stable under the conditions of manufacture and storage, and may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The form depends on the intended mode of administration and therapeutic application. In some embodiments, the disclosed ADCs can be incorporated into a pharmaceutical composition suitable for parenteral administration. The injectable solution may be composed of either a liquid or lyophilized dosage form in a flint or amber vial, ampule, or pre-filled syringe, or other known delivery or storage device. In some embodiments, one or more of the ADCs or pharmaceutical compositions is supplied as a dry sterilized lyophilized powder or water free concentrate in a hermetically sealed container and can be reconstituted (e.g., with water or saline) to the appropriate concentration for administration to a subject.

Typically, a therapeutically effective amount or efficacious amount of a disclosed composition, e.g., a disclosed ADC, is employed in the pharmaceutical compositions of the present disclosure. The composition, e.g., one comprising an ADC, may be formulated into a pharmaceutically acceptable dosage form by conventional methods known in the art. Dosages and administration protocols for the treatment of cancers using the foregoing methods will vary with the method and the target cancer, and will generally depend on a number of other factors appreciated in the art.

Dosage regimens for compositions disclosed herein, e.g., those comprising ADCs alone or in combination with at least one additional inactive and/or active therapeutic agent, may be adjusted to provide the optimum desired response (e.g., a therapeutic response). For example, a single bolus of one or both agents may be administered at one time, several divided doses may be administered over a predetermined period of time, or the dose of one or both agents may be proportionally increased or decreased as indicated by the exigencies of the therapeutic situation. In some embodiments, treatment involves single bolus or repeated administration of the ADC preparation via an acceptable route of administration. In some embodiments, the ADC is administered to the patient daily, weekly, monthly, or any time period in between. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage values for compositions comprising an ADC and/or any additional therapeutic agent(s), may be selected based on the unique characteristics of the active compound(s), and the particular therapeutic effect to be achieved. A physician or veterinarian can start doses of the ADC employed in the pharmaceutical composition at levels lower than that required to achieve the desired therapeutic effect and gradually increase the dosage until the desired effect is achieved. In general, effective doses of the compositions of the present disclosure, for the treatment of a cancer may vary depending upon many different factors, including means of administration, target site, physiological state of the patient, whether the patient is human or an animal, other medications administered, and whether treatment is prophylactic or therapeutic. The selected dosage level may also depend upon a variety of pharmacokinetic factors including the activity of the particular compositions of the present disclosure employed, or the ester, salt, or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Treatment dosages may be titrated to optimize safety and efficacy.

Toxicity and therapeutic efficacy of compounds provided herein can be determined by standard pharmaceutical procedures in cell culture or in animal models. For example, LD50, ED50, EC50, and IC50 may be determined, and the dose ratio between toxic and therapeutic effects (LD50/ED50) may be calculated as the therapeutic index. The data obtained from in vitro and in vivo assays can be used in estimating or formulating a range of dosage for use in humans. For example, the compositions and methods disclosed herein may initially be evaluated in xenogeneic cancer models (e.g., an NCI-H929 multiple myeloma mouse model).

In some embodiments, an ADC or composition comprising an ADC is administered on a single occasion. In other embodiments, an ADC or composition comprising an ADC is administered on multiple occasions. Intervals between single dosages can be, e.g., daily, weekly, monthly, or yearly. Intervals can also be irregular, based on measuring blood levels of the administered agent (e.g., the ADC) in the patient in order to maintain a relatively consistent plasma concentration of the agent. The dosage and frequency of administration of an ADC or composition comprising an ADC may also vary depending on whether the treatment is prophylactic or therapeutic. In prophylactic applications, a relatively low dosage may be administered at relatively infrequent intervals over a long period of time. Some patients continue to receive treatment for the rest of their lives. In therapeutic applications, a relatively higher dosage at relatively shorter intervals is sometimes required until progression of the disease is reduced or terminated, and preferably until the patient shows partial or complete amelioration of one or more symptoms of disease. Thereafter, the patient may be administered a lower, e.g., prophylactic regime.

The above therapeutic approaches can be combined with any one of a wide variety of additional surgical, chemotherapy, or radiation therapy regimens. In some embodiments, the ADCs or compositions disclosed herein are co-formulated and/or co-administered with one or more additional therapeutic agents, e.g., one or more chemotherapeutic agents, one or more standard-of-care agents for the particular condition being treated.

Kits for use in the therapeutic and/or diagnostic applications described herein are also provided. Such kits may comprise a carrier, package, or container that is compartmentalized to receive one or more containers such as vials, tubes, and the like, each of the container(s) comprising one of the separate elements to be used in a method disclosed herein. A label may be present on or with the container(s) to indicate that an ADC or composition within the kit is used for a specific therapy or non-therapeutic application, such as a prognostic, prophylactic, diagnostic, or laboratory application. A label may also indicate directions for either in vivo or in vitro use, such as those described herein. Directions and or other information may also be included on an insert(s) or label(s), which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that an ADC or composition within the kit is used for diagnosing or treating a condition, such as a cancer a described herein.

In some embodiments, a kit comprises an ADC or composition comprising an ADC. In some embodiments, the kit further comprises one or more additional components, including but not limited to: instructions for use; other reagents, e.g., a therapeutic agent (e.g., a standard-of-care agent); devices, containers, or other materials for preparing the ADC for administration; pharmaceutically acceptable carriers; and devices, containers, or other materials for administering the ADC to a subject. Instructions for use can include guidance for therapeutic applications including suggested dosages and/or modes of administration, e.g., in a patient having or suspected of having a cancer. In some embodiments, the kit comprises an ADC and instructions for use of the ADC in treating, preventing, and/or diagnosing a cancer.

It is known that elevated Bcl-xL expression correlates with resistance to radiation therapy and chemotherapy. Antibody-drug conjugates (ADCs) that may not be sufficiently effective as monotherapy to treat cancer can be administered in combination with other therapeutic agents (including non-targeted and targeted therapeutic agents) or radiation therapy (including radioligand therapy) to provide therapeutic benefit. Without wishing to be bound by theory, it is believed that the ADCs described herein sensitize tumor cells to the treatment with other therapeutic agents (including standard of care chemotherapeutic agents to which the tumor cells may have developed resistance) and/or radiation therapy. In some embodiments, antibody drug conjugates described herein, are administered to a subject having cancer in an amount effective to sensitize the tumor cells. As used herein, the term “sensitize” means that the treatment with ADC increases the potency or efficacy of the treatment with other therapeutic agents and/or radiation therapy against tumor cells.

### 3. Combination Therapies

In some embodiments, the present disclosure provides methods of treatment wherein the antibody-drug conjugates disclosed herein are administered in combination with one or more (e.g., 1 or 2) additional therapeutic agents. Exemplary combination partners are disclosed herein.

In certain embodiments, a combination described herein comprises a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDIO680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab.

In certain embodiments, a combination described herein comprises a LAG-3 inhibitor. In some embodiments, the LAG-3 inhibitor is chosen from LAG525 (Novartis), BMS-986016 (Bristol-Myers Squibb), or TSR-033 (Tesaro).

In certain embodiments, a combination described herein comprises a TIM-3 inhibitor. In some embodiments, the TIM-3 inhibitor is MBG453 (Novartis), TSR-022 (Tesaro), LY-3321367 (Eli Lily), Sym23 (Symphogen), BGB-A425 (Beigene), INCAGN-2390 (Agenus), BMS-986258 (BMS), RO-7121661 (Roche), or LY-3415244 (Eli Lilly).

In certain embodiments, a combination described herein comprises a PDL1 inhibitor. In one embodiment, the PDL1 inhibitor is chosen from FAZ053 (Novartis), atezolizumab (Genentech), durvalumab (Astra Zeneca), or avelumab (Pfizer).

In certain embodiments, a combination described herein comprises a GITR agonist. In some embodiments, the GITR agonist is chosen from GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

In some embodiments, a combination described herein comprises an IAP inhibitor. In some embodiments, the IAP inhibitor comprises LCL161 or a compound disclosed in International Application Publication No. WO 2008/016893.

In an embodiment, the combination comprises an mTOR inhibitor, e.g., RADO01 (also known as everolimus).

In an embodiment, the combination comprises a HDAC inhibitor, e.g., LBH589. LBH589 is also known as panobinostat.

In an embodiment, the combination comprises an IL-17 inhibitor, e.g., CJM112.

In certain embodiments, a combination described herein comprises an estrogen receptor (ER) antagonist. In some embodiments, the estrogen receptor antagonist is used in combination with a PD-I inhibitor, a CDK4/6 inhibitor, or both. In some embodiments, the combination is used to treat an ER positive (ER+) cancer or a breast cancer (e.g., an ER+breast cancer).

In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9(7): 465-479). ER is a hormone-activated transcription factor important for e.g., the growth, development and physiology of the human reproductive system. ER is activated by, e.g., the hormone estrogen (17beta estradiol). ER expression and signaling is implicated in cancers (e.g., breast cancer), e.g., ER positive (ER+) breast cancer. In some embodiments, the SERD is chosen from LSZ102, fulvestrant, brilanestrant, or elacestrant.

In some embodiments, the SERD comprises a compound disclosed in International Application Publication No. WO 2014/130310, which is hereby incorporated by reference in its entirety.

In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid. In some embodiments, the SERD comprises fulvestrant (CAS Registry Number: 129453-61-8), or a compound disclosed in International Application Publication No. WO 2001/051056, which is hereby incorporated by reference in its entirety. In some embodiments, the SERD comprises elacestrant (CAS Registry Number: 722533-56-4), or a compound disclosed in U.S. Pat. No. 7,612,114, which is incorporated by reference in its entirety. Elacestrant is also known as RAD1901, ER-306323 or (6R)-6-{2-[Ethyl({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26(9):948-56. In some embodiments, the SERD is brilanestrant (CAS Registry Number: 1365888-06-7), or a compound disclosed in International Application Publication No. WO 2015/136017, which is incorporated by reference in its entirety.

In some embodiments, the SERD is chosen from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58(12) 4883-4887.

Other exemplary estrogen receptor antagonists are disclosed, e.g., in WO 2011/156518, WO 2011/159769, WO 2012/037410, WO 2012/037411, and US 2012/0071535, all of which are hereby incorporated by reference in their entirety

In certain embodiments, a combination described herein comprises an inhibitor of Cyclin-Dependent Kinases 4 or 6 (CDK4/6). In some embodiments, the CDK4/6 inhibitor is used in combination with a PD-1 inhibitor, an estrogen receptor (ER) antagonist, or both. In some embodiments, the combination is used to treat an ER positive (ER+) cancer or a breast cancer (e.g., an ER+breast cancer). In some embodiments, the CDK4/6 inhibitor is chosen from ribociclib, abemaciclib (Eli Lilly), or palbociclib.

In some embodiments, the CDK4/6 inhibitor comprises ribociclib (CAS Registry Number: 1211441-98-3), or a compound disclosed in U.S. Pat. Nos. 8,415,355 and 8,685,980, which are incorporated by reference in their entirety.

In some embodiments, the CDK4/6 inhibitor comprises a compound disclosed in International Application Publication No. WO 2010/020675 and U.S. Pat. Nos. 8,415,355 and 8,685,980, which are incorporated by reference in their entirety.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4- [4-fluoro-2-methyl-1-(1-methylethyl)-1H-benzimidazol-6-yl]-2-pyrimidinamine. Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl-5-methyl-2-{[5-(1-piperazinyl)-2-pyridinyl]amino}pyrido[2,3-d]pyrimidin-7(8H)-one. Palbociclib inhibits CDK4 with an IC50 of 11 nM, and inhibits CDK6 with an IC50 of 16 nM, and is disclosed, e.g., in Finn et al. (2009) Breast Cancer Research 11(5):R77.

In certain embodiments, a combination described herein comprises an inhibitor of chemokine (C—X-C motif) receptor 2 (CXCR2). In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide, danirixin, reparixin, or navarixin.

In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

In certain embodiments, a combination described herein comprises a c-MET inhibitor. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein. In some embodiments, the c-MET inhibitor is chosen from capmatinib (INC280), JNJ-3887605, AMG 337, LY2801653, MSC2156119J, crizotinib, tivantinib, or golvatinib.

In certain embodiments, a combination described herein comprises a transforming growth factor beta (also known as TGF-β TGFβ, TGFb, or TGF-beta, used interchangeably herein) inhibitor. In some embodiments, the TGF-β inhibitor is chosen from fresolimumab or XOMA 089.

In certain embodiments, a combination described herein comprises an adenosine A2a receptor (A2aR) antagonist (e.g., an inhibitor of A2aR pathway, e.g., an adenosine inhibitor, e.g., an inhibitor of A2aR or CD-73). In some embodiments, the A2aR antagonist is used in combination with a PD-1 inhibitor, and one or more (e.g., two, three, four, five, or all) of a CXCR2 inhibitor, a CSF-1/1R binding agent, LAG-3 inhibitor, a GITR agonist, a c-MET inhibitor, or an IDO inhibitor. In some embodiments, the combination is used to treat a pancreatic cancer, a colorectal cancer, a gastric cancer, or a melanoma (e.g., a refractory melanoma). In some embodiments, the A2aR antagonist is chosen from PBF509 (NIR178) (Palobiofarma/Novartis), CPI444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), or Preladenant/SCH 420814 (Merck/Schering). Without wishing to be bound by theory, it is believed that in some embodiments, inhibition of A2aR leads to upregulation of IL-lb.

In certain embodiments, a combination described herein comprises an inhibitor of indoleamine 2,3-dioxygenase (IDO) and/or tryptophan 2,3-dioxygenase (TDO). In some embodiments, the IDO inhibitor is used in combination with a PD-1 inhibitor, and one or more (e.g., two, three, four, or all) of a TGF-β inhibitor, an A2aR antagonist, a CSF-1/1R binding agent, a c-MET inhibitor, or a GITR agonist. In some embodiments, the combination is used to treat a pancreatic cancer, a colorectal cancer, a gastric cancer, or a melanoma (e.g., a refractory melanoma). In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod (NLG8189), (1-methyl-D-tryptophan), a-cyclohexyl-5H-Imidazo[5,1-a]isoindole-5-ethanol (also known as NLG919), indoximod, BMS-986205 (formerly F001287).

In certain embodiments, a combination described herein comprises a Galectin, e.g., Galectin-1 or Galectin-3, inhibitor. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is used in combination with one or more therapeutic agents described herein. In some embodiments, the Galectin inhibitor is chosen from an anti-Galectin antibody molecule, GR-MD-02 (Galectin Therapeutics), Galectin-3C (Mandal Med), Anginex, or OTX-008 (OncoEthix, Merck).

In some embodiments, a combination described herein comprises an inhibitor of the MAP kinase pathway including ERK inhibitors, MEK inhibitors and RAF inhibitors.

In some embodiments, a combination described herein comprises a MEK inhibitor. In some embodiments, the MEK inhibitor is chosen from Trametinib, selumetinib, AS703026, BIX 02189, BIX 02188, CI-1040, PD0325901, PD98059, U0126, XL-518, G-38963, or G02443714.

In some embodiments, the MEK inhibitor is trametinib. Trametinib is also known as JTP-74057, TMT212, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, or Mekinist (CAS Number 871700-17-3).

In some embodiments, the MEK inhibitor comprises selumetinib which has the chemical name: (5-[(4-bromo-2-chlorophenyl)amino]-4-fluoro-N-(2-hydroxyethoxy)-1-methyl-iH-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436).

In some embodiments, the MEK inhibitor comprises N-[(2R)-2,3-Dihydroxypropoxy]-3,4-difluoro-2-[(2-fluoro-4-iodophenyl)amino]- benzamide (also known as PD0325901), e.g., as described in PCT Publication No. WO2002006213).

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis [amino [(2-aminophenyl)thio]methylene]-butanedinitrile (also known as U0126), e.g., as described in U.S. Pat. No. 2,779,780).

In some embodiments, the MEK inhibitor comprises XL-518 (also known as GDC-0973) which has a CAS No. 1029872-29-4 and is available from ACC Corp.

In some embodiments, the MEK inhibitor comprises G-38963.

In some embodiments, the MEK inhibitor comprises G02443714 (also known as AS703206)

Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, 2,3-Bis [amino [(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione](also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido [2,3-d]pyrimidine-4,7(3H,8H)-dione (TAK-733, CAS 1035555-63-5); pimasertib (AS—703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, a combination described herein comprises a RAF inhibitor.

RAF inhibitors include, but are not limited to, Vemurafenib (or Zelboraf®, PLX-4032, CAS 918504-65-1), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®).

In some embodiments, the RAF inhibitor is Dabrafenib.

In some embodiments, the RAF inhibitor is LXH254.

In some embodiments, a combination described herein comprises an ERK inhibitor.

ERK inhibitors include, but are not limited to, LTT462, ulixertinib (BVD-523), LY3214996, GDC-0994, KO—947 and MK-8353.

In some embodiments, the ERK inhibitor is LTT462. LTT462 is 4-(3-amino-6-((1S,3S,4S)-3-fluoro-4-hydroxy-cyclohexyl)pyrazin-2-yl)-N-((S)-1-(3-bromo-5-fluorophenyl)-2-(methylamino)-ethyl)-2-fluorobenzamide and is the compound of the following structure:

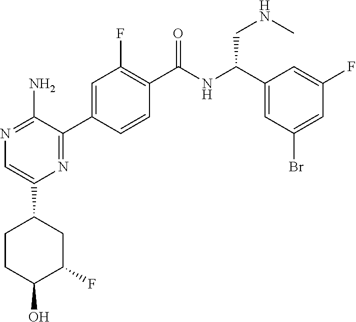 [SMILES: CNC[C@@H](NC(=O)c1ccc(-c2nc([C@H]3CC[C@H](O)[C@@H](F)C3)cnc2N)cc1F)c1cc(F)cc(Br)c1]

The preparation of LTT462 is described in PCT patent application publication WO2015/066188. LTT462 is an inhibitor of extracellular signal-regulated kinases 1 and 2 (ERK 1/2).

In some embodiments, a combination described herein comprises a taxane, a vinca alkaloid, a MEK inhibitor, an ERK inhibitor, or a RAF inhibitor.

In some embodiments, a combination described herein comprises at least two inhibitors selected, independently, from a MEK inhibitor, an ERK inhibitor, and a RAF inhibitor.

In some embodiments, a combination described herein comprises an anti-mitotic drug.

In some embodiments, a combination described herein comprises a taxane.

Taxanes include, but are not limited to, docetaxel, paclitaxel, or cabazitaxel. In some embodiments, the taxane is docetaxel.

In some embodiments, a combination described herein comprises a vinca alkaloid.

Vinca alkaloids include, but are not limited to, vincristine, vinblastine, and leurosine.

In some embodiments, a combination described herein comprises a topoisomerase inhibitor.

Topoisomerase inhibitors include, but are not limited to, topotecan, irinotecan, camptothecin, diflomotecan, lamellarin D, ellipticines, etoposide (VP-16), teniposide, doxorubicin, daunorubicin, mitoxantrone, amsacrine, aurintricarboxylic acid, and HU-331.

In one embodiment, a combination described herein includes an interleukin-1 beta (IL-1(3) inhibitor. In some embodiments, the IL-10 inhibitor is chosen from canakinumab, gevokizumab, Anakinra, or Rilonacept.

In certain embodiments, a combination described herein comprises an IL-15/IL-15Ra complex. In some embodiments, the IL-15/IL-15Ra complex is chosen from NIZ985 (Novartis), ATL-803 (Altor) or CYP0150 (Cytune).

In certain embodiments, a combination described herein comprises a mouse double minute 2 homolog (MDM2) inhibitor. The human homolog of MDM2 is also known as HDM2. In some embodiments, an MDM2 inhibitor described herein is also known as a HDM2 inhibitor. In some embodiments, the MDM2 inhibitor is chosen from HDM201 or CGMO97.

In an embodiment the MDM2 inhibitor comprises (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (also known as CGMO97) or a compound disclosed in PCT Publication No. WO 2011/076786 to treat a disorder, e.g., a disorder described herein). In one embodiment, a therapeutic agent disclosed herein is used in combination with CGMO97.

In some embodiments, a combination described herein comprises a hypomethylating agent (HMA). In some embodiments, the HMA is chosen from decitabine or azacitidine.

In some embodiments, a combination described herein comprises a glucocorticoid. In some embodiments, the glucocorticoid is dexamethasone.

In some embodiments, a combination described herein comprises asparaginase.

In certain embodiments, a combination described herein comprises an inhibitor acting on any pro-survival proteins of the Bcl2 family. In certain embodiments, a combination described herein comprises a Bcl-2 inhibitor. In some embodiments, the Bcl-2 inhibitor is venetoclax (also known as ABT-199):

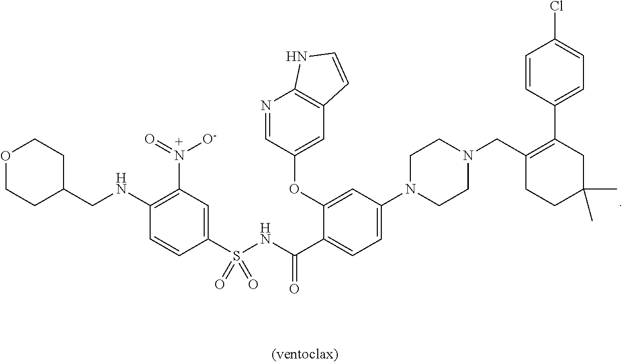

In one embodiment, the Bcl-2 inhibitor is selected from the compounds described in WO 2013/110890 and WO 2015/011400. In some embodiments, the Bcl-2 inhibitor comprises navitoclax (ABT-263), ABT-737, BP1002, SPC2996, APG-1252, obatoclax mesylate (GX15-070MS), PNT2258, Zn-d5, BGB-11417, or oblimersen (G3139). In some embodiments, the Bcl-2 inhibitor is N-(4-hydroxyphenyl)-3-[6-[(3S)-3-(morpholinomethyl)-3,4-dihydro-1H-isoquinoline-2-carbonyl]-1,3-benzodioxol-5-yl]-N-phenyl-5,6,7,8-tetrahydroindolizine-1-carboxamide, compound A1:

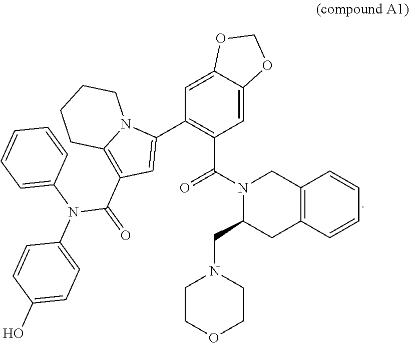 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

In some embodiments, the Bcl-2 inhibitor is (S)-5-(5-chloro-2-(3-(morpholinomethyl)-1,2,3,4-tetrahydroisoquinoline-2-carbonyl)phenyl)-N-(5-cyano-1,2-dimethyl-1H-pyrrol-3-yl)-N-(4-hydroxyphenyl)-1,2-dimethyl-1H-pyrrole-3-carboxamide), compound A2:

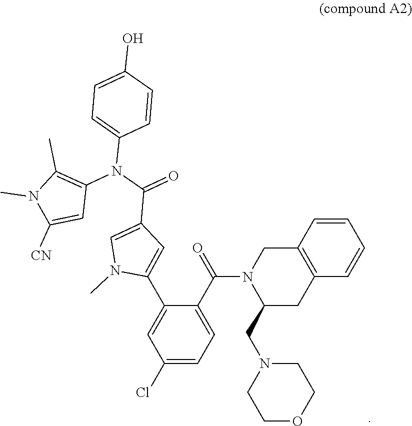 [SMILES: Cc1c(N(C(=O)c2cc(-c3cc(Cl)ccc3C(=O)N3Cc4ccccc4C[C@H]3CN3CCOCC3)n(C)c2)c2ccc(O)cc2)cc(C#N)n1C]

In one embodiment, the antibody-drug conjugates or combinations disclosed herein are suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a hormone therapy (e.g., with anti-estrogens or anti-androgens), a radiation therapy, surgery, or any other therapeutic agent or modality, to treat a disorder herein. For example, to achieve antigen-specific enhancement of immunity, the combination can be administered together with an antigen of interest. A combination disclosed herein can be administered in either order or simultaneously.

### 4. Additional Embodiments

The disclosure provides the following additional embodiments for linker-drug groups, antibody-drug conjugates, linker groups, and methods of conjugation.

### Linker-Drug Group

In some embodiments, the Linker-Drug group of the invention may be a compound having the structure of Formula (A′), or a pharmaceutically acceptable salt thereof:

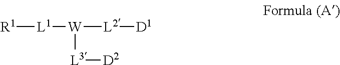

wherein:
R¹ is a reactive group;
L¹ is a bridging spacer;
W is branching moiety;
L^{2′} and L^{3′}, are each independently a linker;
D¹ and D² are each independently an antineoplastic compound, wherein at least one of D¹ and
D² is a BH3 mimetic.

In some embodiments, the R^{1′} group in Formula (A′) is R¹⁰⁰ described herein. In some embodiments, R^{1′} is a reactive group depicted in Table F.

Certain aspects and examples of the Linker-Drug group or Linker-Drug compound of the invention are provided in the following listing of enumerated embodiments. It will be recognized that features specified in each embodiment may be combined with other specified features to provide further embodiments of the present invention.
Embodiment 1. The compound of Formula (A′), or pharmaceutically acceptable salt thereof, wherein:
   R^{1′} is a reactive group;
   L¹ is a bridging spacer;
   W is branching moiety;
   L^{2′} and L^{3′}, are each independently a linker;
   D¹ and D² are each independently an antineoplastic compound, wherein at least one of D¹ and
   D² is a BH3 mimetic. Alternatively, D¹ and D² are each independently a BH3 mimetic.

In some embodiments, each of L^{2′} and L³′ comprises a cleavable group, optionally wherein at least one cleavable group comprises a glucuronide group, pyrophosphate group, a peptide group, and/or a self-immolative group. Alternatively, each of L^{2′} and L” comprises a cleavable group, optionally wherein at least one cleavable group comprises a pyrophosphate group, a peptide group, and/or a self-immolative group.
Embodiment 2. The compound of Embodiment 1, or pharmaceutically acceptable salt thereof, wherein the compound is presented by Formula (B′),

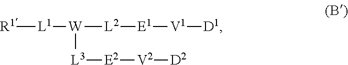

wherein:
R^{1′} is reactive group;
L¹ is a bridging spacer;
W is N or CR^{W}; wherein R^{W} is H or C₁₋₆alkyl;
L² and L³ are each independently a connecting spacer;
E¹ and E² are each independently, an enzyme cleavage element or a hydrophilic moiety;
V¹ and V² are each independently comprise i) a self immolative group, ii) an enzyme cleavage element, or iii) a self immolative group and an enzyme cleavage element. Alternatively, V¹ and
V² are each independently i) a self immolative group, ii) an enzyme cleavage element.
Embodiment 3. The compound of Embodiment 2, or pharmaceutically acceptable salt thereof, wherein (i) V¹ and V² each independently comprises a phosphate, a pyrophosphate and/or a self-immolative group; (ii) V¹ and V² each independently comprises a self-immolative group; (iii) V¹ and V² each independently comprises a self-immolative group comprising —CH₂—O—, —OC(═O)—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium; or (iv) V¹ and V² each independently comprises a group comprising para-aminobenzyl-phosphate or para-aminobenzyl-pyrophosphate. Alternatively, for the compound of Embodiment 2, or pharmaceutically acceptable salt thereof, wherein (i) V¹ and V² each independently comprises a phosphate, a pyrophosphate and/or a self-immolative group; (ii) V¹ and V² each independently comprises a self-immolative group; or (iii) V¹ and V² each independently comprises a self-immolative group comprising —CH₂—O—, —OC(═O)—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.
Embodiment 4. The compound of Embodiment 1, or pharmaceutically acceptable salt thereof, wherein the compound is represented by Formula (C′):

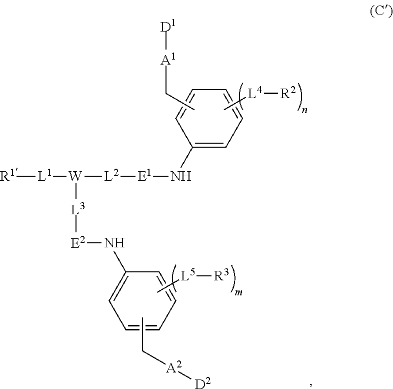

wherein R^{1′} is a reactive group, and the remaining variables depicted therein as defined in the antibody-drug conjugate of Formula (C) described in the seventh embodiment above or any embodiment described therein.
Embodiment 5. The compound of Embodiment 4, or pharmaceutically acceptable salt thereof, wherein the compound is represented by Formula (D1′), (D2′) or (D3′):

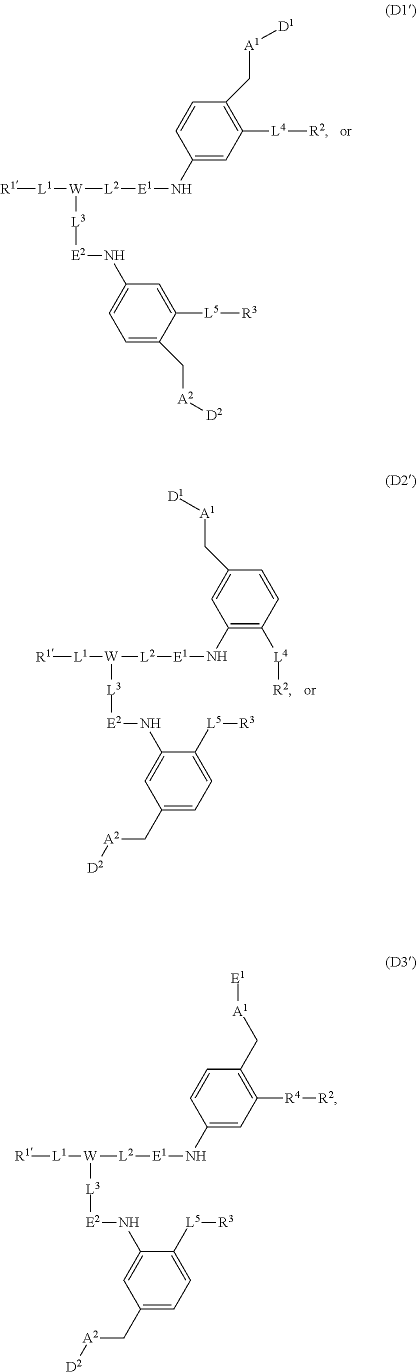

or pharmaceutically acceptable salt thereof, wherein for Formula (D2′), R² and R³ are each independently an enzyme cleavage element; and for Formula (D3′), R² is a hydrophilic group and R³ is an enzyme cleavage element.
Embodiment 6. The compound of Embodiment 5, or pharmaceutically acceptable salt thereof, wherein for Formula (D1′), R² and R³ are each independently a hydrophilic group.
Embodiment 7. The compound of any one of Embodiments 1 to 6, or pharmaceutically acceptable salt thereof, wherein:
   R^{1′} is: a thiol,
   a maleimide,
   a haloacetamide,
   an azide,
   an alkyne,
   a cyclcooctene,
   a triaryl phosphine,
   an oxanobornadiene,
   a cyclooctyne,
   a diaryl tetrazine,
   a monoaryl tetrazine,
   a norbornene,
   an aldehyde,
   a hydroxylamine,
   a hydrazine,
   NH₂—NH—C(═O)—,
   a ketone,
   a vinyl sulfone,
   an aziridine,
   an amino acid residue. In some embodiments, R¹⁷ is:

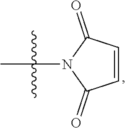 [SMILES: *N1C(=O)C=CC1=O]

—ONH₂, —NH₂,

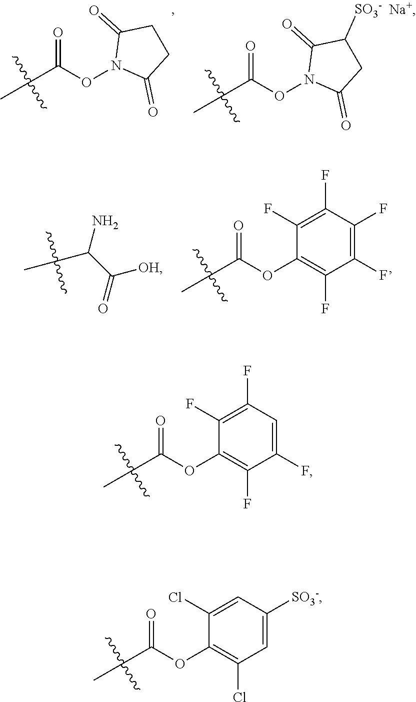

### —N₃,

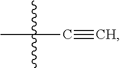 [SMILES: *C#C]

—SH, —SR₃, —SSR⁴, —S(═O)₂(CH═CH₂), —(CH₂)₂S(═O)₂(CH═CH₂), —NHS(═O)₂(CH═CH₂), —NHC(═O)CH₂Br, -NHC(═O)CH₂I,

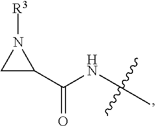

### —C(O)NHNH₂,

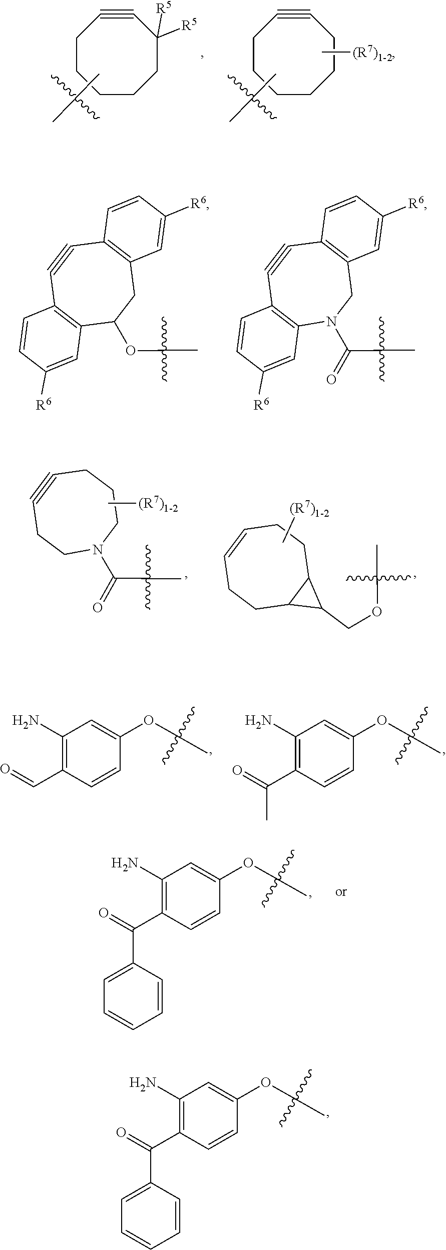

wherein:
each R¹¹ is independently selected from H and C₁-C₆alkyl;
each R¹² is 2-pyridyl or 4-pyridyl;
each R¹³ is independently selected from H, C₁-C₆alkyl, F, Cl, and —OH;
each R¹⁴ is independently selected from H, C₁-C₆alkyl, F, Cl, —NH₂, —OCH₃, —OCH₂CH₃, —N(CH₃)₂, —CN, —NO₂ and —OH;
each R¹⁵ is independently selected from H, C_{1 6}alkyl, fluoro, benzyloxy substituted with —C(═O)OH, benzyl substituted with —C(═O)OH, C₁₋₄alkoxy substituted with —C(═O)OH and C_{1 4}alkyl substituted with —C(═O)OH.
Embodiment 8. The compound of Embodiment 7, or a pharmaceutically acceptable salt thereof, wherein R^{1′} is

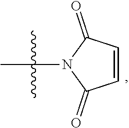 [SMILES: *N1C(=O)C=CC1=O]

Embodiment 9. The compound of any one of Embodiments 1 to 8, or pharmaceutically acceptable salt thereof, wherein L¹ is as defined in the fourteenth embodiment for the antibody-drug conjugate described above.
Embodiment 10. The compound of any one of Embodiments 1 to 9, or pharmaceutically acceptable salt thereof, wherein L¹ is as defined in the fifteenth embodiment for the antibody-drug conjugate described above.
Embodiment 11. The compound of Embodiments 10, or pharmaceutically acceptable salt thereof, wherein L¹ is as defined in the sixteenth embodiment for the antibody-drug conjugate described above.
Embodiment 12. The compound of any one of Embodiments 1 to 11, or pharmaceutically acceptable salt thereof, wherein L¹ is selected from (L1-1), (L1-2), (L1-3), (L1-4), (L1-5) and (L1-6) as defined in the seventeenth embodiment for the antibody-drug conjugate described above.
Embodiment 13. The compound of any one of Embodiments 1 to 12, or pharmaceutically acceptable salt thereof, wherein L² and L³ are each independently a connecting spacer comprising a moiety represented by formula (L2a) as defined in the eighteenth embodiment for the antibody-drug conjugate described above.
Embodiment 14. The compound of Embodiment 13, or pharmaceutically acceptable salt thereof, wherein L² and L³ are each independently a connecting spacer selected from (L2b)-(L2m) as defined in the nineteenth embodiment for the antibody-drug conjugate described above.
Embodiment 15. The compound of Embodiment 14, or pharmaceutically acceptable salt thereof, wherein L² and L³ are each independently a connecting spacer selected from (L2AA)-(L2SS) as defined in the twentieth embodiment for the antibody-drug conjugate described above.
Embodiment 16. The compound of Embodiment 15, or pharmaceutically acceptable salt thereof, wherein L² and L³, independently, are a connecting spacer selected from a group consisting of (L2-1) to (L2-30) as defined in the twenty-first embodiment for the antibody-drug conjugate described above.
Embodiment 17. The compound of Embodiment 16, or pharmaceutically acceptable salt thereof, wherein d is 25.
Embodiment 18. The compound of any one of Embodiments 4 to 17, or pharmaceutically acceptable salt thereof, wherein the peptide group comprises 1 to 4, 1 to 3, or 1 to 2 amino acid residues.
Embodiment 19. The compound of Embodiment 18, or pharmaceutically acceptable salt thereof, wherein the amino acid residues are selected from L-glycine (Gly), L-valine (Val), L-citrulline (Cit), L-cysteic acid (sulfo-Ala), L-lysine (Lys), L-isoleucine (Ile), L-phenylalanine (Phe), L-methionine (Met), L-asparagine (Asn), L-proline (Pro), L-alanine (Ala), L-leucine (Leu), L-tryptophan (Trp), L-tyrosine (Tyr) and (3-alanine (0-Ala).
Embodiment 20. The compound of any one of Embodiments 4 to 17, or pharmaceutically acceptable salt thereof, wherein the peptide group comprises Val-Cit, Phe-Lys, Val-Ala, Val-Lys, Leu-Cit, Cit-((3-Ala), Gly-Gly-Gly, Gly- Gly-Phe-Gly, and/or sulfo-Ala-Val-Ala.
Embodiment 21. The compound of any one of Embodiments 18 to 20, or pharmaceutically acceptable salt thereof, wherein the peptide group represented by E¹ or E² is an enzyme cleavage element.
Embodiment 22. The compound of any one of Embodiments 18 to 20, or pharmaceutically acceptable salt thereof, wherein the peptide group represented by E¹ or E² is a hydrophilic moiety.
Embodiment 23. The compound of Embodiment 21, or pharmaceutically acceptable salt thereof, wherein E¹ or E², independently, is an enzyme cleavage element selected from a group consisting of

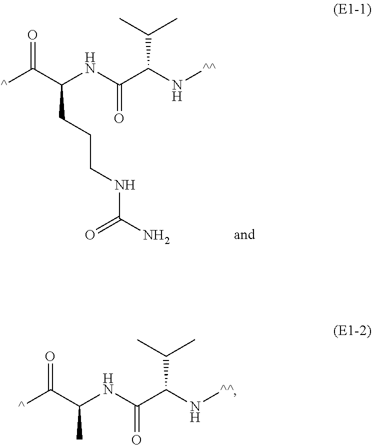

wherein \hat{ } of E¹ or E² indicates the point of direct attachment to V¹ or V² in Formula (B′) or direct attachment to the —NH— group in Formula (C′) and (D′); and of E¹ or E² indicates the point of direct attachment to L² or L³, respectively
Embodiment 24. The compound of Embodiment 22, or pharmaceutically acceptable salt thereof, wherein E¹ or E², independently, is a hydrophilic moiety represented by

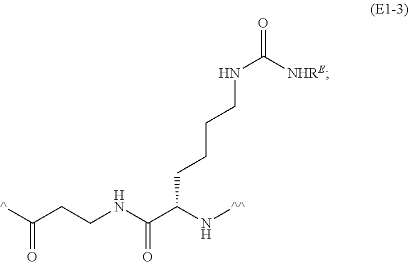

wherein R^{E} is a hydrophilic group R^{H}.
Embodiment 25. The compound of Embodiment 24, or pharmaceutically acceptable salt thereof,

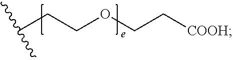

wherein each hydrophilic group R^{H} in E¹ or E² is independently e; wherein e is an integer between 20 and 30 (e.g., 20, 21, 22, 23, 24, 25, 26, 27, 28, 29 or 30).
Embodiment 26. The compound of Embodiment 25, or pharmaceutically acceptable salt thereof, wherein e is 24.
Embodiment 27. The compound of any one of Embodiments 4 to 26, or pharmaceutically

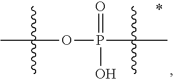 [SMILES: *OP(*)(=O)O]

acceptable salt thereof, wherein A¹ and A² independently are a bond, —OC(═O)—*, or OH wherein * indicates the point of attachment to D¹ or D². In some embodiments, A¹ and A² independently are a bond or

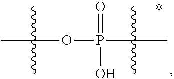 [SMILES: *OP(*)(=O)O]

wherein * indicates the point of attachment to D¹ or D².

In some embodiments, A¹ and A² independently are a bond or —OC(═O)—*, wherein * indicates the point of attachment to D¹ or D². In some embodiments, A¹ and A² are: (i) A¹ and A² are —OC(═O)—*; (ii) A¹ and A² are

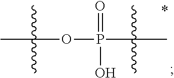 [SMILES: *OP(*)(=O)O]

(iii) A¹ is —OC(═O)—* and A² is a bond; (iv) A1 is —OC(═O)—* and A²is

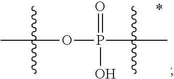 [SMILES: *OP(*)(=O)O]

(v) A is a bond and A² is

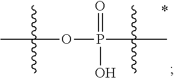 [SMILES: *OP(*)(=O)O]

or (vi) A¹ is a bond and A² is —OC(═O)—*, wherein * indicates the point of attachment to D¹ or D².
Embodiment 28. The compound of Embodiment 27, or pharmaceutically acceptable salt thereof, wherein A¹ and A²are a bond.
Embodiment 29. The compound of any one of Embodiments 4 to 28, or pharmaceutically acceptable salt thereof, wherein L⁴ and L⁵ as defined in the thirty-fourth embodiment for the antibody-drug conjugate described above.
Embodiment 30. The compound of Embodiment 29, or pharmaceutically acceptable salt thereof, wherein Z is —O—, —CH₂NR^{L45} C(═O)—, —CH₂NR^{L45}C(═O)NH— or —CH₂O—; X is a bond, triazolyl, or —CH₂-triazolyl-; and R^{L45}, in each occurrence, is independently H or C_{1 3}alkyl.
Embodiment 31. The compound of any one of Embodiments 4 to 30, or pharmaceutically acceptable salt thereof, wherein L⁴ and L⁵are each independently a spacer moiety selected from a group consisting of

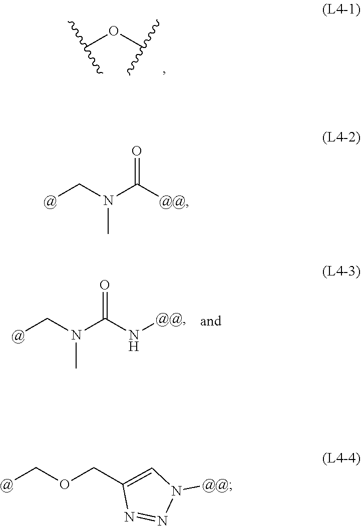

wherein the @ of L⁴or L⁵ indicates the point of direct attachment to the phenyl group, and the @@ of L⁴ or L⁵ indicates the point of direct attachment to R² or R³.
Embodiment 32. The compound of any one of Embodiments 4 to 31, or pharmaceutically acceptable salt thereof, wherein the hydrophilic moieties represented by R² and R³ each independently comprises polyethylene glycol, polyalkylene glycol, a polyol, a polysarcosine, a sugar, an oligosaccharide, a polypeptide, C₂-C₆ alkyl substituted with 1 to 3

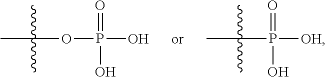 [SMILES: *OP(=O)(O)O ; *P(=O)(O)O]

or C₂—C₆alkyl substituted with 1 to 2 substituents independently selected from —OC(═O)NHS(O)₂NHCH₂CH₂OCH₃, —NHC(═O)C_{1 4}alkylene-P(O)(OCH₂CH₃)₂ and —COOH groups.
Embodiment 33. The compound of any one of Embodiments 4 to 32, or pharmaceutically acceptable salt thereof, wherein R² or R³ independently is

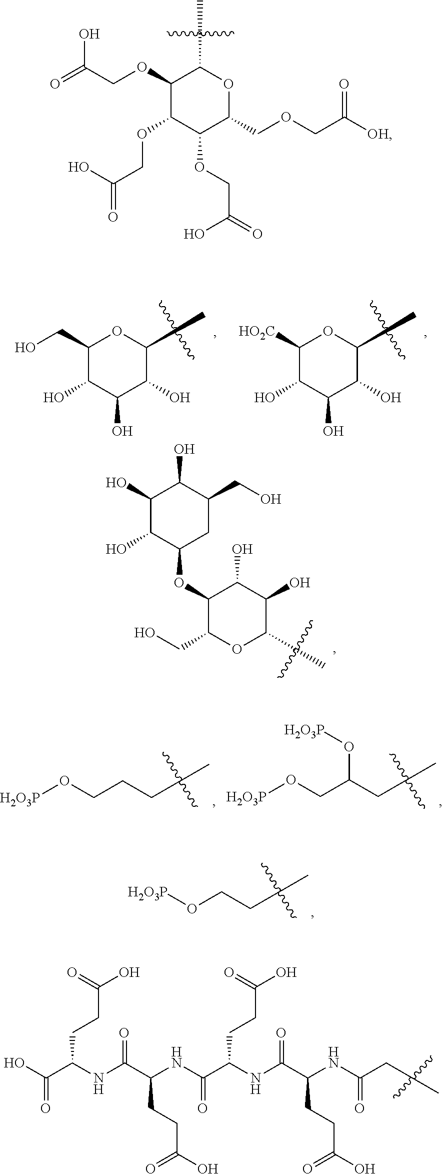

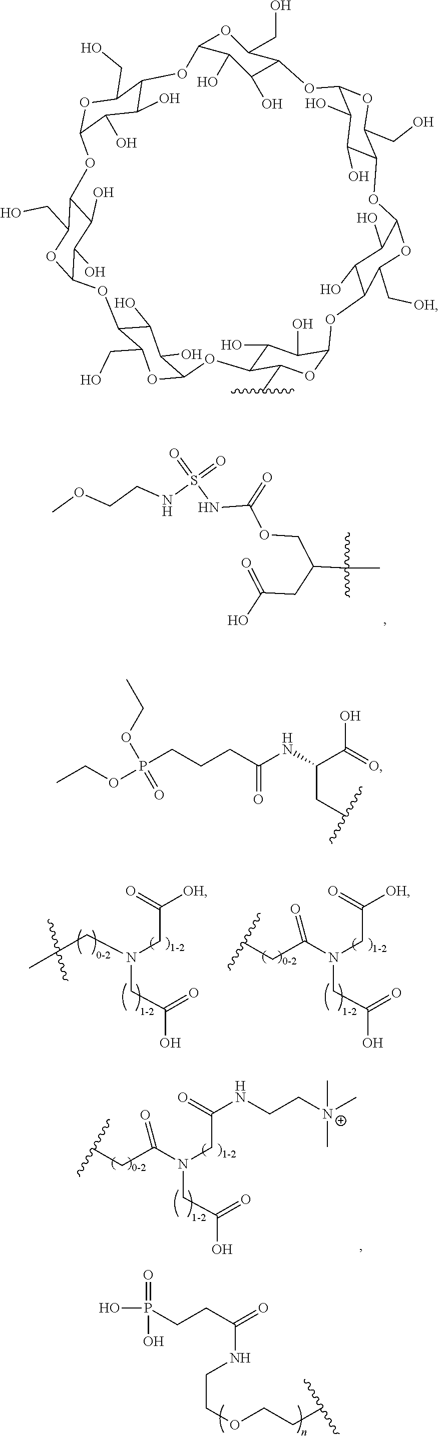

wherein n is an integer between 1 and 6,

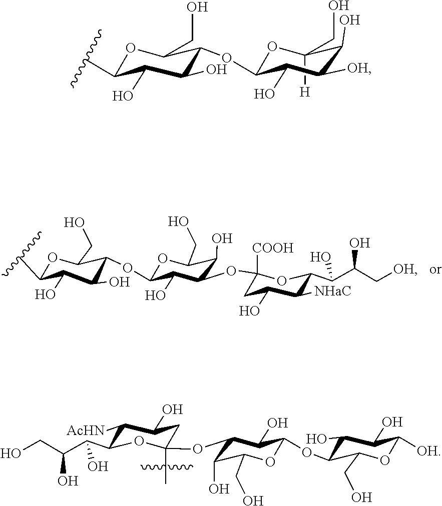

Embodiment 34. The compound of any one of Embodiments 4 to 33, or pharmaceutically acceptable salt thereof, wherein the hydrophilic group represented by R² or R³ each independently comprises:
(i) a polysarcosine with the following moiety:

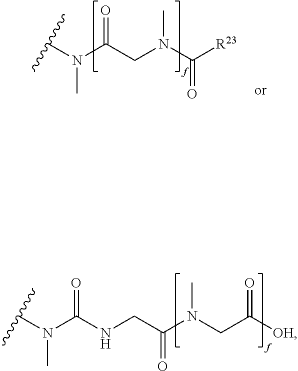

f is an integer between 3 and 25; and
R²³ is H, —CH₃ or —CH₂CH₂C(═O)OH; or
(ii) a polyethylene glycol of formula:

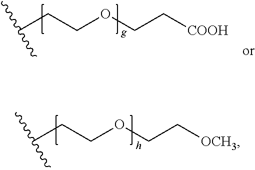

wherein
g and h are independently an integer between 2 and 30.

In some embodiments, the hydrophilic group represented by R2 or R3 each independently comprises:
(i) a polysarcosine with the following moiety:

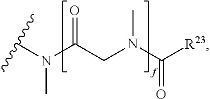

wherein
f is an integer between 3 and 25; and R²³ is H, —CH₃ or —CH₂CH₂C(═O)OH.
Embodiment 35. The compound of any one Embodiments 4 to 31, or pharmaceutically acceptable salt thereof, wherein the enzyme cleavage element represented by R² or R³ each independently comprises:

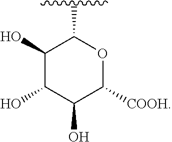 [SMILES: *[C@@H]1O[C@H](C(=O)O)[C@@H](O)[C@H](O)[C@H]1O]

Embodiment 36. The compound of any one of Embodiments 4 to 31, or pharmaceutically acceptable salt thereof, wherein the R² or R³, independently, is selected from a group consisting of

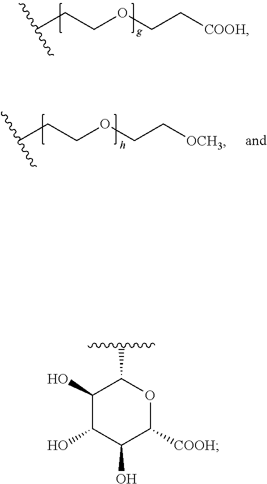

wherein g and h are independently an integer between 20 and 30.
Embodiment 37. The compound of any one of Embodiment 34 or 36, or pharmaceutically acceptable salt thereof, wherein g is 23, 24, or 25; and h is 23, 24, or 25.
Embodiment 38. The compound of Embodiment 4, or pharmaceutically acceptable salt thereof, wherein the dual linker is represented by following formula:

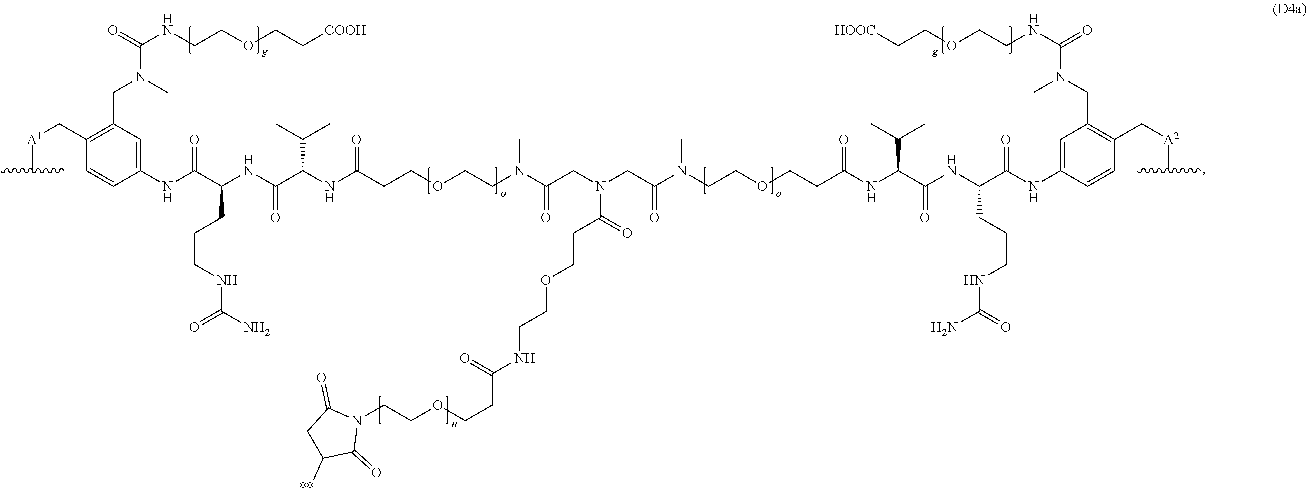

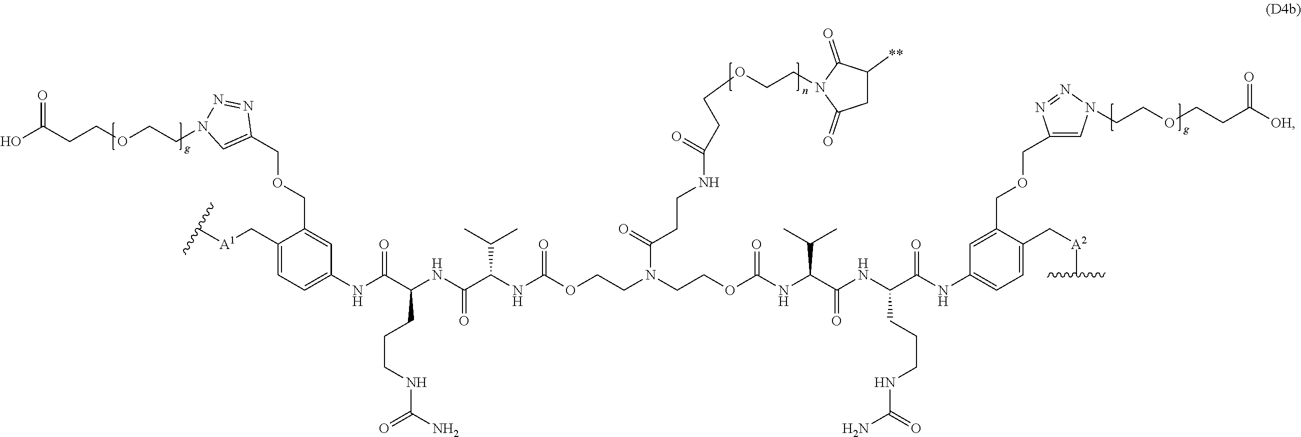

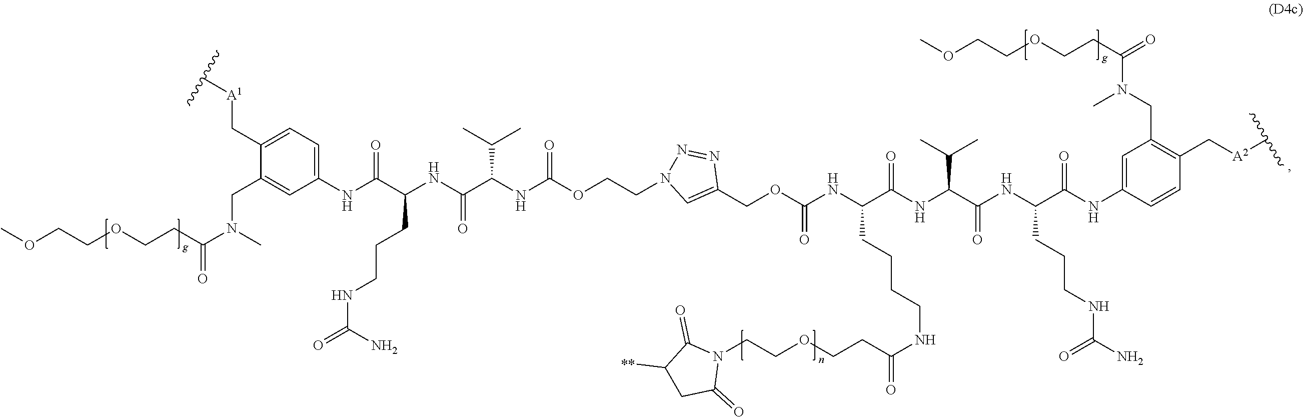

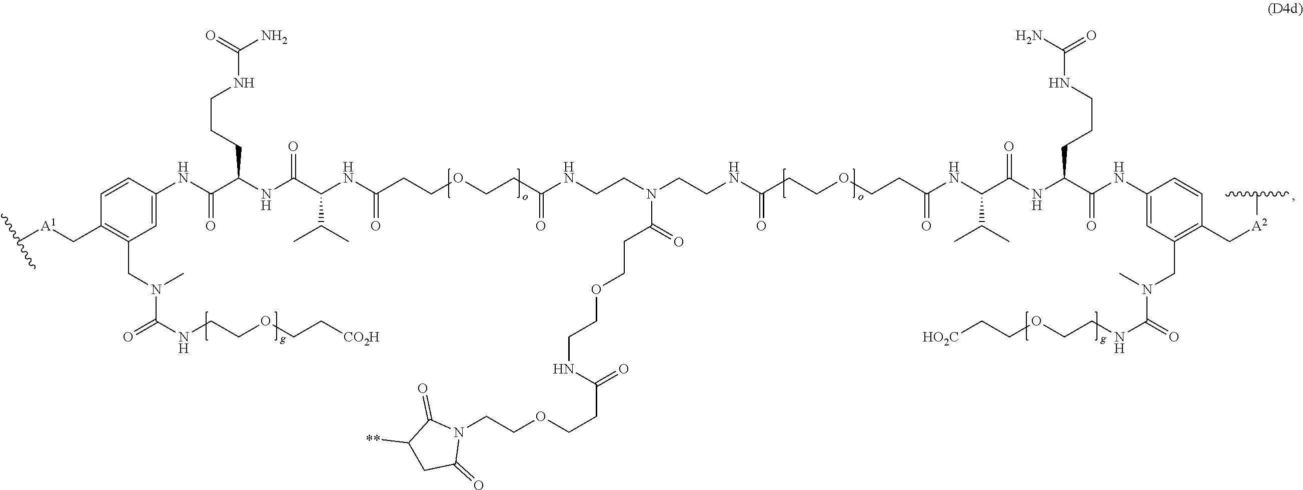

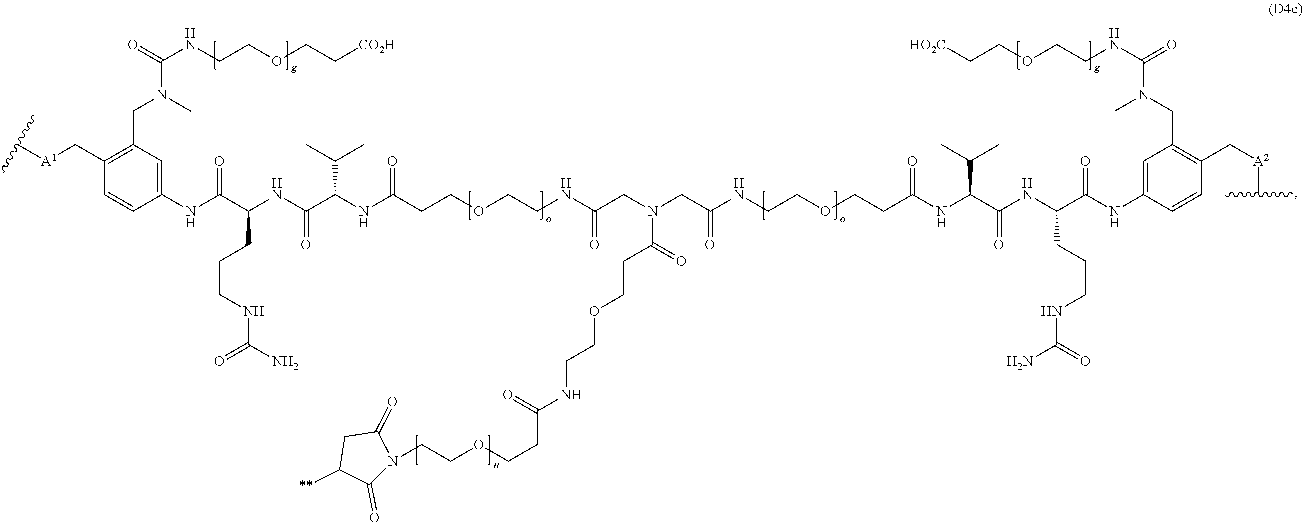

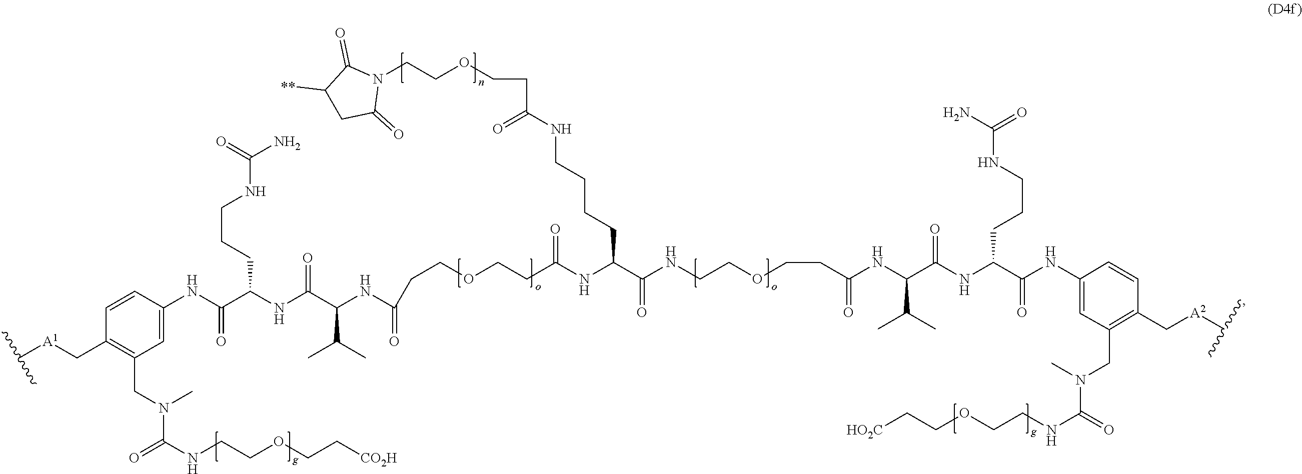

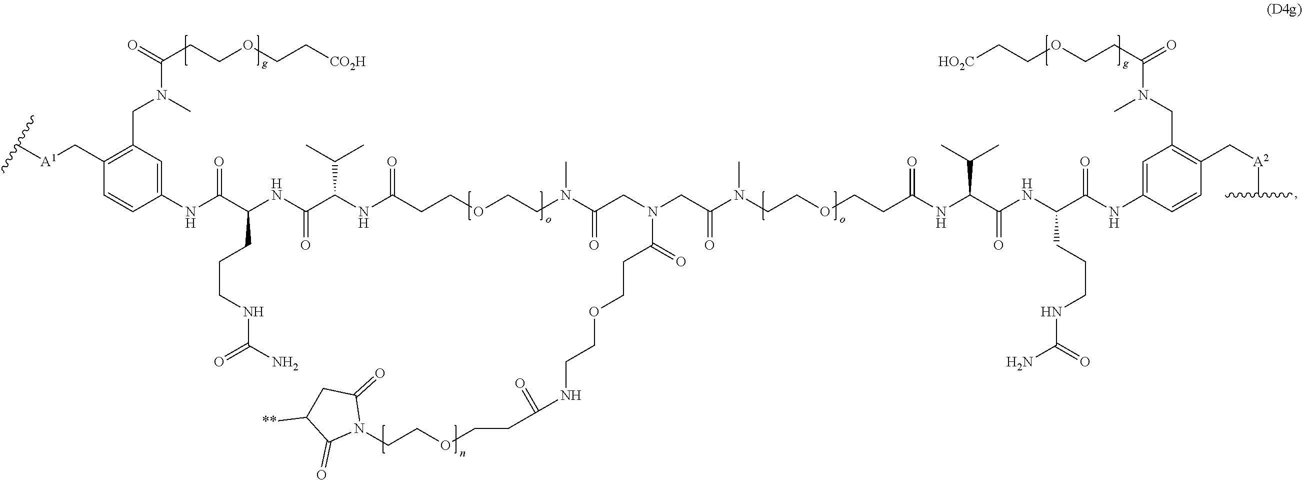

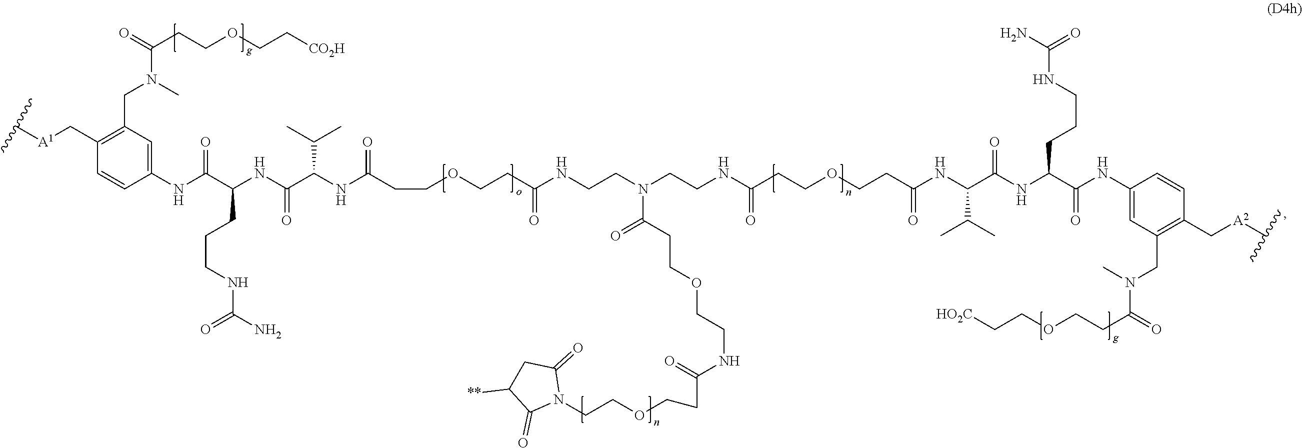

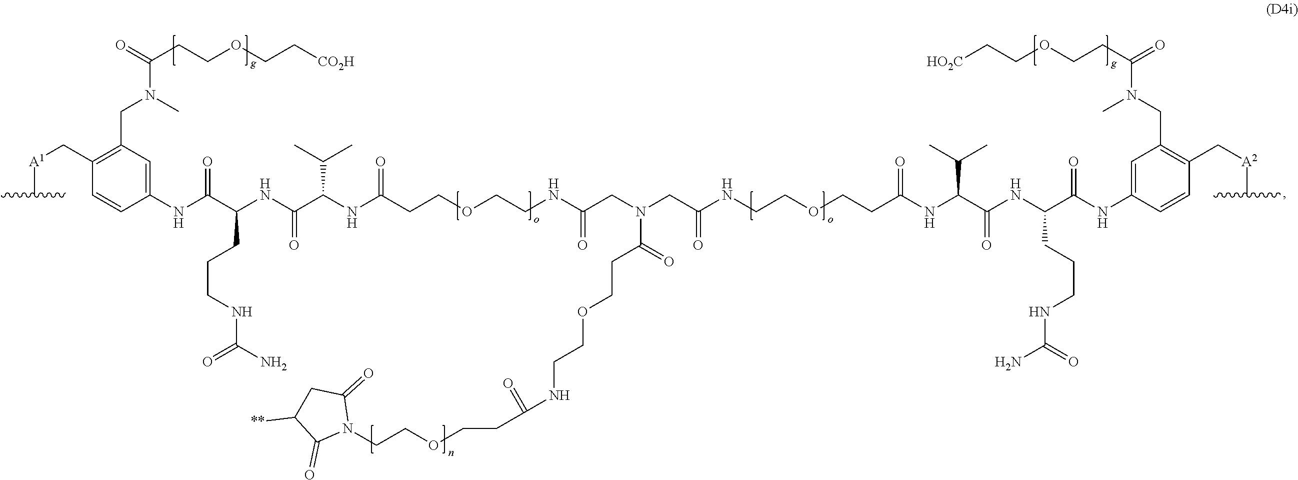

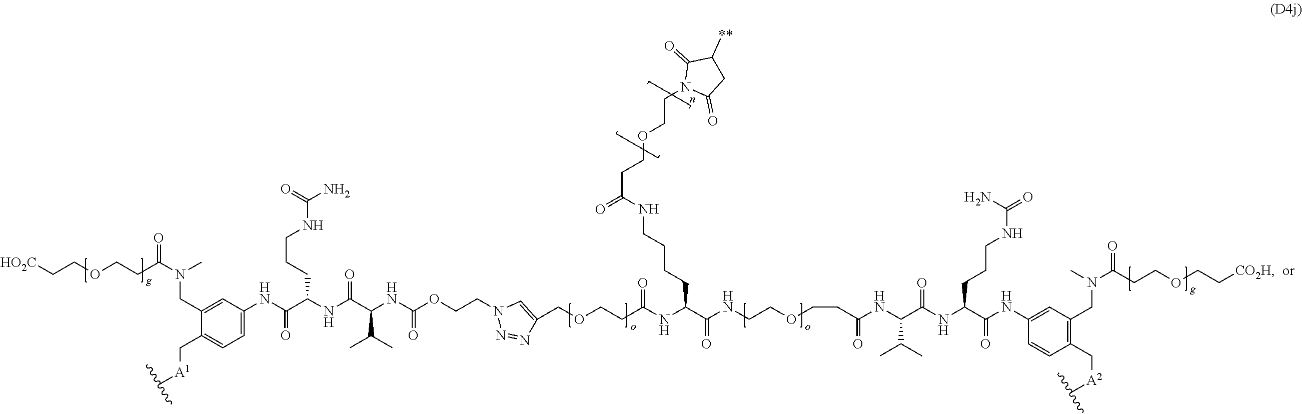

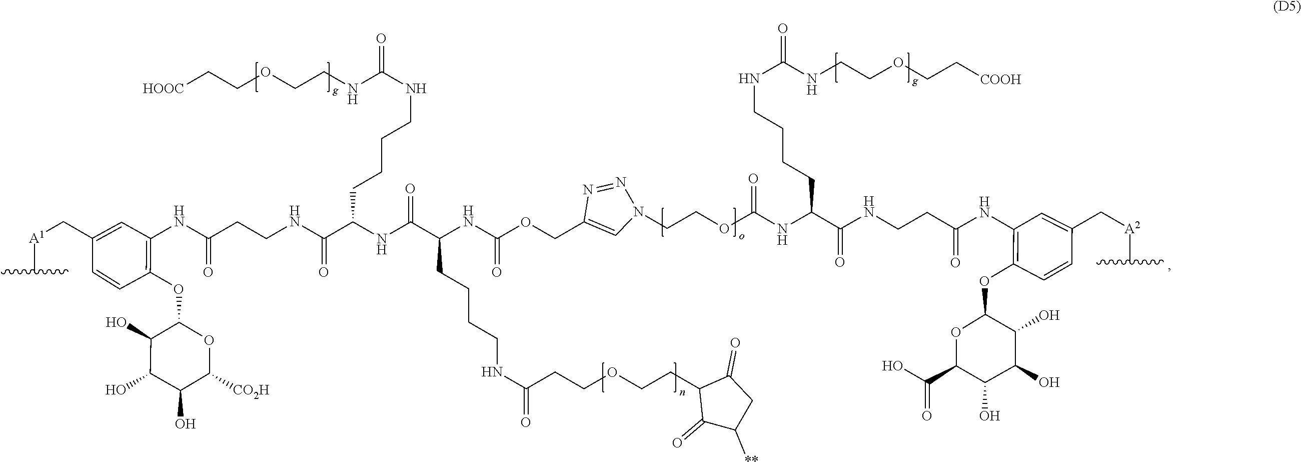

wherein:
A¹ and A² are each independent a bond, —O—C(═O)—* or

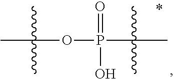 [SMILES: *OP(*)(=O)O]

wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 2 and 5);
n is an integer between 1 and 12 (e.g., between 2 and 5);
[symbol] indicates the point of attachment to the Ab; and
[symbol] indicates the point of direct attachment to D¹ or D².

In some embodiments A¹ and A² are each independent a bond or —O—C(═O)—*. In some embodiments, A¹ and A² are both a bond. In some embodiments, A¹ and A² are both —O—C(═O)—*. In some embodiments, one of A¹ and A² is a bond, and the other is —O—C(═O)—*.
Embodiment 39. The compound of Embodiment 1, or pharmaceutically acceptable salt thereof, wherein the dual linker is represented by any one of (L1)-(L36) as defined in the forty-fourth embodiment for the antibody-drug conjugate described above, in which A¹ and A² are each independent a bond, —O—C(═O)—* or

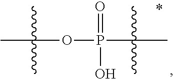 [SMILES: *OP(*)(=O)O]

wherein * in A¹ and A² indicates the point of attachment to D¹ or D². In some embodiments, A¹ and A² are each independently a bond or —O—C(═O)—*. In some embodiments, A¹ and A² are both a bond. In some embodiments, A¹ and A² are both —O—C(═O)—*. In some embodiments, one of A¹ and A² is a bond, and the other is —O—C(═O)—*. Embodiment 40. The compound of any one of Embodiments 1 to 39, or pharmaceutically acceptable salt thereof, wherein D¹ and D² are each independently a BH3 mimetic. In some embodiments, D¹ and D² are each independently selected from a Mcl-1 inhibitor, a Bcl-2 inhibitor and a Bcl-xL inhibitor. Alternatively, for the compound of any one of Embodiments 1 to 39, or pharmaceutically acceptable salt thereof, one of D¹ and D² is a BH3 mimetic selected from a Mcl-1 inhibitor, a Bcl-2 inhibitor, and a Bcl-xL inhibitor, and the other is an antineoplastic non-BH3 mimetic selected from topoisomerase 1 inhibitor or an anti-mitotic drug. In some embodiments, for the compound of any one of Embodiments 1 to 39, or pharmaceutically acceptable salt thereof, D¹ is a BH3 mimetic and D² is an antineoplastic non-BH3 mimetic. In some embodiments, D, is selected from a Mcl-1 inhibitor, a Bcl-2 inhibitor, and a Bcl-xL inhibitor, and D² is a topoisomerase 1 inhibitor or an anti-mitotic drug. In some embodiments, D¹ is a Bcl-xL inhibitor and D² is a topoisomerase 1 inhibitor. In some embodiments, D¹ is a Bcl-xL inhibitor and D² is an anti-mitotic drug.
Embodiment 41. The compound of any one of Embodiments 1 to 40, or pharmaceutically acceptable salt thereof, wherein D¹ and D² are both (i) a Mcl-1 inhibitor; (ii) a Bcl-2 inhibitor; or (iii) Bcl-xL inhibitor.
Embodiment 42. The compound of any one of Embodiments 1 to 41, or pharmaceutically acceptable salt thereof, wherein D¹ and D² are the same.
Embodiment 43. The compound of any one of Embodiments 1 to 41, or pharmaceutically acceptable salt thereof, wherein D¹ and D² are different.
Embodiment 44. The compound of any one of Embodiments 1 to 39, or pharmaceutically acceptable salt thereof, wherein (i) one of D¹ and D² is a Mcl-1 inhibitor and the other is a Bcl-2 inhibitor; (ii) one of D¹ and D²is a Mcl-1 inhibitor and the other is a Bcl-xL inhibitor; or (iii) one of D¹ and D² is a Bcl-2 inhibitor and the other is a Bcl-xL inhibitor. In some embodiments, D, and D² are as defined: (i) D¹ is a Mcl-1 inhibitor and D² is a Mcl-1 inhibitor; (ii) D¹ is a Mcl-1 inhibitor and D² is a Bcl-2 inhibitor; (iii) D¹ is a Bcl-xL inhibitor and D² is a Bcl-xL inhibitor: (iv) D¹ is a Bcl-xL inhibitor and D² is a Bcl-2 inhibitor; or (v) D¹ is a Bcl-2 inhibitor and D² is a Mcl-1 inhibitor; or (vi) D¹ is a Mcl-1 inhibitor and D² is a Bcl-xL inhibitor.
Embodiment 45. The compound of any one of Embodiments 40 to 44, or pharmaceutically acceptable salt thereof, wherein the Mcl-1 inhibitor is represented by Formula (I) as defined in the fiftieth or fifty-first embodiment for the antibody-drug conjugate described above.
Embodiment 46. The compound of Embodiment 45, or pharmaceutically acceptable salt thereof, wherein the Mcl-1 inhibitor is represented by Formula (IA) as defined in the fifty-second embodiment for the antibody-drug conjugate described above.
Embodiment 47. The compound of Embodiment 45, or pharmaceutically acceptable salt thereof, wherein the Mcl-1 inhibitor is represented by Formula (IB) as defined in the fifty-third, fifty-fourth, fifty-fifth, fifty-sixth, fifty-seventh, fifty-eighth, fifty-ninth, sixtieth or sixty-first embodiment for the antibody-drug conjugate described above.
Embodiment 48. The compound of Embodiment 45 to 47, or pharmaceutically acceptable salt thereof, wherein the Mcl-1 inhibitor is attached by a covalent bond to R₀₃ of formula (I), (IA), or (IB); or is attached by a covalent bond to R₀₉ of formula (I), (IA), or (IB).
Embodiment 49. The compound of Embodiment 45, or pharmaceutically acceptable salt thereof, wherein the Mcl-1 inhibitor is represented by any one of the Formulas in Table A1 defined in the sixty-third embodiment for the antibody-drug conjugate described above.
Embodiment 50. The compound of any one of Embodiments 40 to 49, or pharmaceutically acceptable salt thereof, wherein the Bcl-xL inhibitor is represented by Formula (II) or Formula (III) as defined in the sixty-fourth embodiment for the antibody-drug conjugate described above.
Embodiment 51. The compound of Embodiment 50, or pharmaceutically acceptable salt thereof, wherein the Bcl-xL inhibitor is represented by Formula (IIA) or (IIIA) as defined in the sixty-fifth, sixty-sixth, sixty-seventh, or sixty-eighty embodiment described for the for the antibody-drug conjugate described above.
Embodiment 52. The compound of Embodiment 50, or pharmaceutically acceptable salt thereof, wherein the Bcl-xL inhibitor is represented by Formula (IIB), (IIC), (IIIB) or (IIIC) as defined in the sixty-ninth embodiment for the antibody-drug conjugate described above.
Embodiment 53. The compound of any one of Embodiments 50 to 52, or pharmaceutically acceptable salt thereof, wherein R₇ represents the following group:

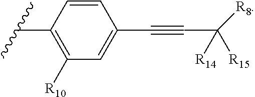

Embodiment 54. The compound of any one of Embodiments 50 to 52, or pharmaceutically acceptable salt thereof, wherein R₇ represents a group selected from:

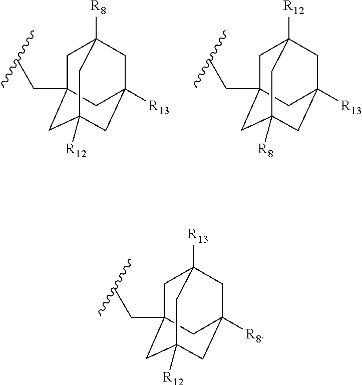

Embodiment 55. The compound of any one of Embodiments 50 to 54, or pharmaceutically acceptable salt thereof, wherein Rs represents a group selected from:

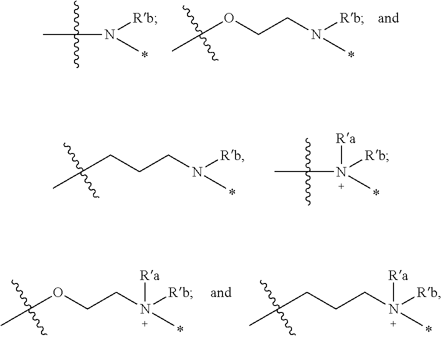

wherein [symbol] represents a bond to the linker.
Embodiment 56. The compound of any one of Embodiments 50 to 54, or pharmaceutically acceptable salt thereof, wherein B₃ represents a C₃-C₅ heterocycloalkyl group selected from a pyrrolidinyl group, a piperidinyl group, a piperazinyl group, a morpholinyl group, an azepanyl group, and a 4,4-difluoropiperidin-1-yl group.
Embodiment 57. The compound of Embodiment 50, or pharmaceutically acceptable salt thereof, wherein the Bcl-xL inhibitor is represented by any one of the formulae depicted in Table A2 in the seventy-fourth embodiment for the antibody-drug conjugate above.
Embodiment 58. The compound of any one of Embodiments 40 to 57, or pharmaceutically acceptable salt thereof, wherein Bcl-2 inhibitor is represented by Formula (IV) or Formula (V) or an enantiomer, a diastereoisomer, and/or a pharmaceutically acceptable salt of any one of the foregoing as defined in the seventy-fifth, seventy-sixth, seventy-seventh, seventy-eighth, seventy-ninth or eightieth embodiment for the antibody-drug conjugate described above.
Embodiment 59. The compound of Embodiment 58, or pharmaceutically acceptable salt thereof, wherein the Bcl-2 inhibitor is represented by Formula (Va) or an enantiomer, a diastereoisomer, and/or a pharmaceutically acceptable salt of any one of the foregoing as defined in the eighty-first embodiment for the antibody-drug conjugate described above.
Embodiment 60. The compound of Embodiment 58 or 59, or pharmaceutically acceptable salt thereof, wherein R₃ in Formula (V) or (Va) represents the following group:

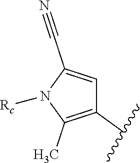

and R_{c} represents a group selected from: hydrogen, linear or branched (C₁-C₆)alkyl group optionally substituted by 1 to 3 halogen atoms, (C₁-C₆)alkylene-NRₐRₑ, (C₁-C₆)alkylene-ORⱼ, cycloalkyl, heterocycloalkyl, and (C₁-C₆)alkylene-heterocycloalkyl group.
Embodiment 61. The compound of Embodiment 60, or pharmaceutically acceptable salt thereof, wherein R_{c} represents a methyl group.
Embodiment 62. The compound of any one of Embodiments 58 to 61, or pharmaceutically acceptable salt thereof, wherein R₄ in Formula (V) or (Va) represents the following group:

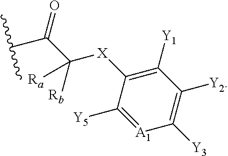

Embodiment 63. The compound of Embodiment 58, or pharmaceutically acceptable salt thereof, wherein the Bcl-2 inhibitor is represented by Formula (Vb) or an enantiomer, a diastereoisomer, and/or a pharmaceutically acceptable salt of any one of the foregoing as defined in the eighty-fifth or eighty-sixth embodiment for the antibody-drug conjugate described above.
Embodiment 64. The compound of Embodiment 58, or pharmaceutically acceptable salt thereof, wherein the Bcl-2 inhibitor is represented by Formula (Vc), (Vd), (Ve), (Vf), (Vg), (Vh), (Vi) or (Vj) as defined in the eighty-seventh or eighty-eighth embodiment for the antibody-drug conjugate described above or any embodiments described therein. In some embodiments, in Formula (V), (Va), (Vb), (Vc), (Vd), (Ve), (Vf), (Vg), (Vh), (Vi) or (Vj), R₅ represents a hydroxy group and R₆ represents a hydrogen atom. In some embodiments, in Formula (V), (Va), (Vb), (Vc), (Vd), (Ve), (Vf), (Vg), (Vh), (Vi) or (Vj), Y₃ represents a —O—(C₁-C₄)alkylene-Cy₃ group.
Embodiment 65. The compound of Embodiment 58, or pharmaceutically acceptable salt thereof, wherein the Bcl-2 inhibitor is represented by any one of the formulae in Table A3 for the antibody-drug conjugate described above.
Embodiment 66. The compound of any one of Embodiments 1 to 40 and 44, or pharmaceutically acceptable salt thereof, wherein the topoisomerase 1 inhibitor is represented by any one of the formulae in Table A4 for the antibody-drug conjugate described above.
Embodiment 67. The compound of any one of Embodiments 1 to 40 and 44 or pharmaceutically acceptable salt thereof, wherein the taxane is selected from docetaxel, paclitaxel, or cabazitaxel

### Methods of Conjugation

The present invention provides various methods of conjugating Linker-Drug groups of the invention to antibodies or antibody fragments to produce Antibody Drug Conjugates which comprise a linker having one or more hydrophilic moieties.

A general reaction scheme for the formation of Antibody Drug Conjugates of Formula (A) is shown in Scheme 1 below:

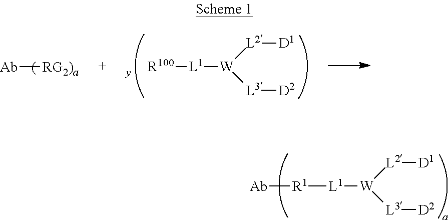

where: R_{G2} is a reactive group which reacts with a compatible R¹⁰⁰ group to form a corresponding R¹ group (such groups are illustrated in Table F and Table G). D¹, D², R¹, L¹, L²′, L^{3′}, Ab, W, a, R¹ and R¹⁰⁰ are as defined herein.

Scheme 2 further illustrates this general approach for the formation of Antibody Drug Conjugates of Formula (Ab2), wherein the antibody comprises reactive groups (R_{G2}) which react with an R¹⁰° group (as defined herein) to covalently attach the Linker-Drug group to the antibody via an R¹ group (as defined herein). For illustrative purposes only Scheme 2 shows the antibody having four R_{G2} groups.

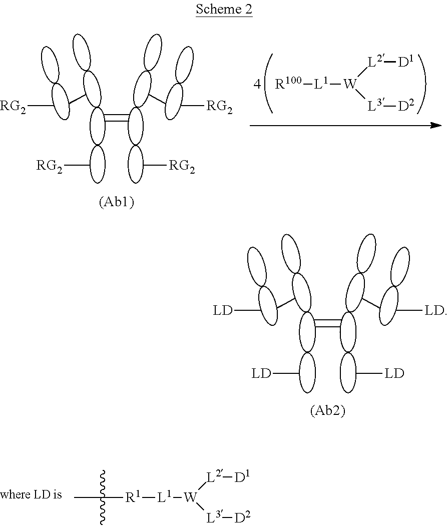

In one aspect, Linker-Drug groups are conjugated to antibodies via modified cysteine residues in the antibodies (see for example WO2014/124316). Scheme 3 illustrates this approach for the formation of Antibody Drug Conjugates of Formula (Ab4) wherein a free thiol group generated from the engineered cysteine residues in the antibody react with an R¹⁰° group (where R⁰⁰ is a maleimide) to covalently attach the Linker-Drug group to the antibody via an R group (where R¹ is a succinimide ring). For illustrative purposes only Scheme 3 shows the antibody having four free thiol groups.

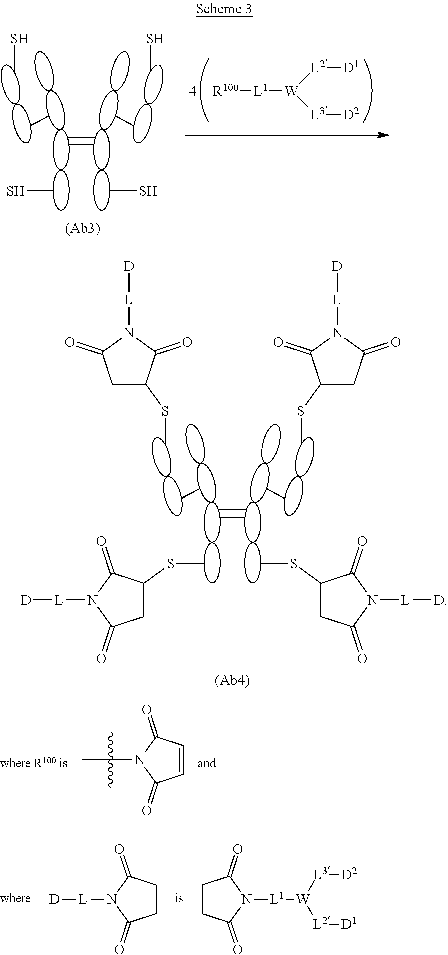

In another aspect, Linker-Drug groups are conjugated to antibodies via lysine residues in the antibodies. Scheme 4 illustrates this approach for the formation of Antibody Drug Conjugates of Formula (Ab6) wherein a free amine group from the lysine residues in the antibody react with an R¹⁰° group (where R¹⁰⁰ is an NHS ester, a pentafluorophenyl or a tetrafluorophenyl) to covalently attach the Linker-Drug group to the antibody via an R¹ group (where R¹ is an amide). For illustrative purposes only Scheme 4 shows the antibody having four amine groups.

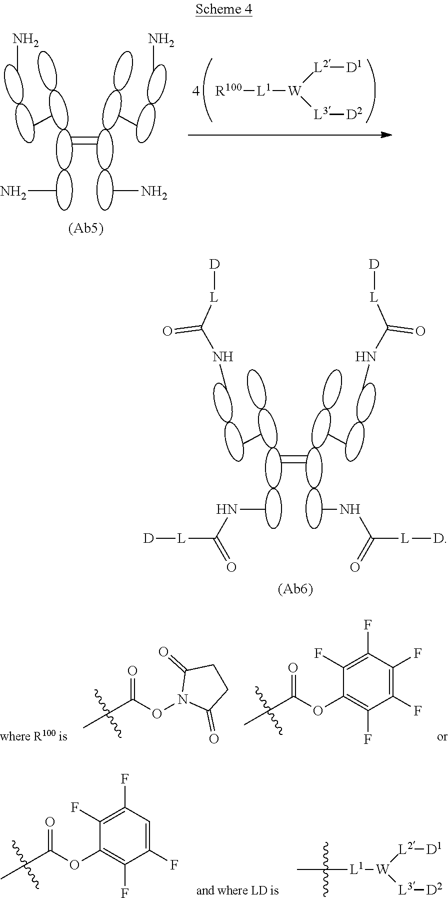

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 5 illustrates this approach for the formation of Antibody Drug Conjugates of Formula (Ab9).

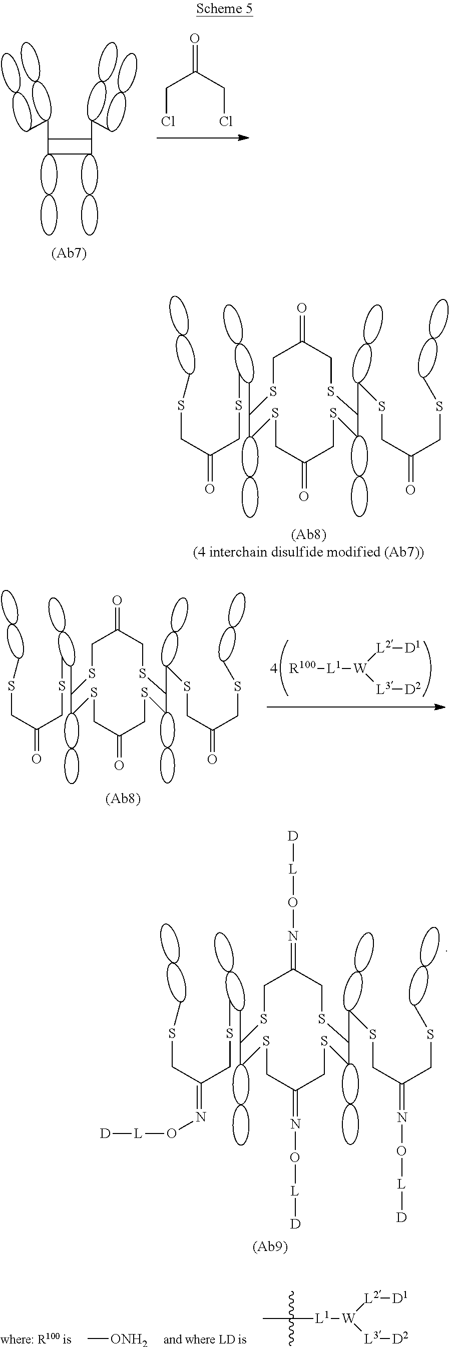

A general reaction scheme for the formation of Antibody Drug Conjugates of Formula (D3) is shown in Scheme 6 below:

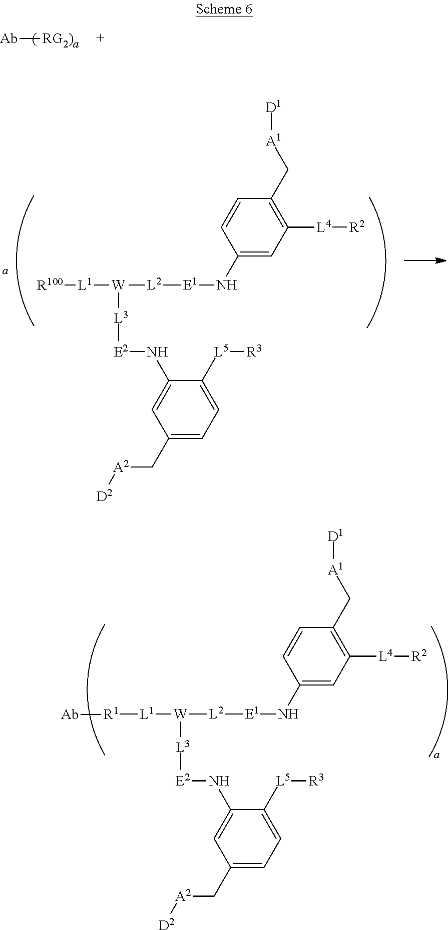

where: R_{G2} is a reactive group which reacts with a compatible R¹⁰⁰ group to form a corresponding R¹ group (such groups are illustrated in Table F and Table G). D¹, D², R¹, L¹, W, L², L³, E¹, E², L⁴, Ls, A¹, A², R², R³, Ab, a and R¹⁰⁰ are as defined herein.

Scheme 7 further illustrates this general approach for the formation of Antibody Drug Conjugates of Formula (Ab11), wherein the antibody comprises reactive groups (R_{G2}) which react with an R¹⁰° group (as defined herein) to covalently attach the Linker-Drug group to the antibody via an R¹ group (as defined herein). For illustrative purposes only Scheme 7 shows the antibody having four R_{G2} groups.

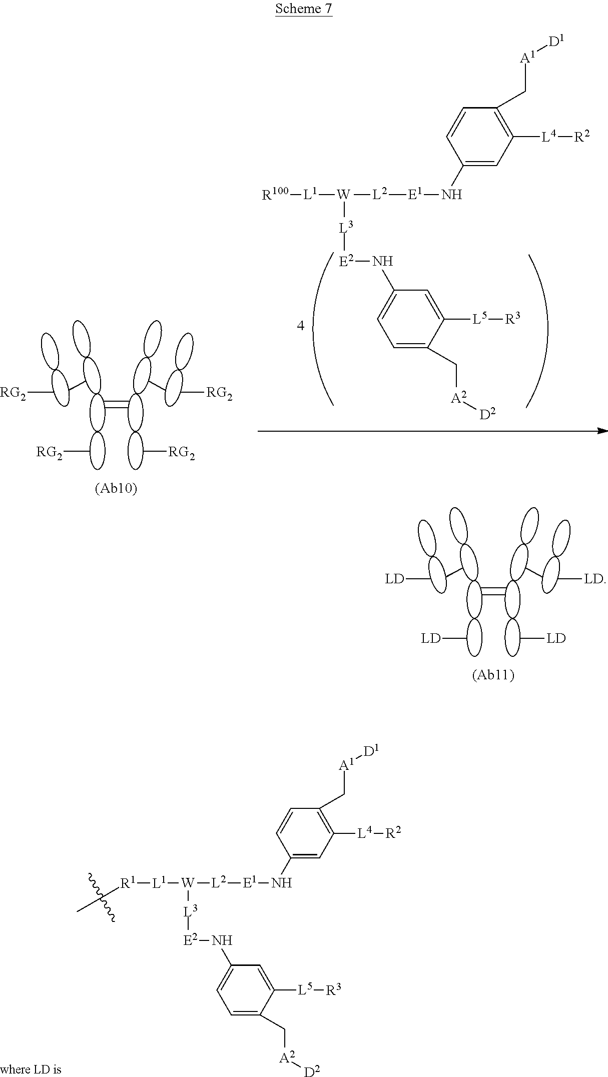

In one aspect, Linker-Drug groups are conjugated to antibodies via modified cysteine residues in the antibodies (see for example WO2014/124316). Scheme 8 illustrates this approach for the formation of Antibody Drug Conjugates of Formula (Ab13) wherein a free thiol group generated from the engineered cysteine residues in the antibody react with an R¹⁰° group (where R¹ is a maleimide) to covalently attach the Linker-Drug group to the antibody via an R group (where R¹ is a succinimide ring). For illustrative purposes only Scheme 8 shows the antibody having four free thiol groups.

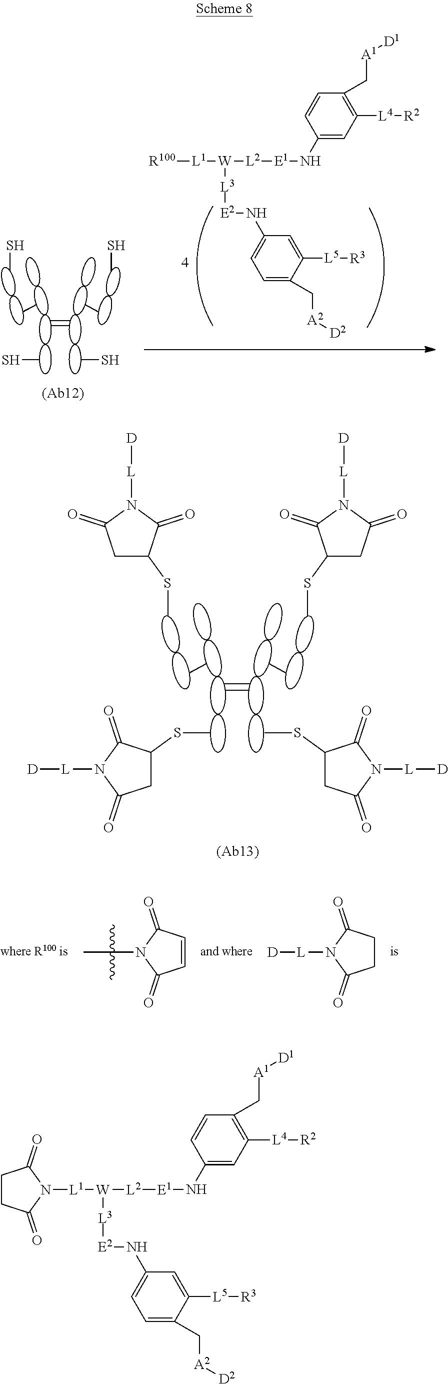

In another aspect, Linker-Drug groups are conjugated to antibodies via lysine residues in the antibodies. Scheme 9 illustrates this approach for the formation of Antibody Drug Conjugates of Formula (Ab15) wherein a free amine group from the lysine residues in the antibody react with an R¹⁰° group (where R¹⁰⁰ is an NHS ester, a pentafluorophenyl or a tetrafluorophenyl) to covalently attach the Linker-Drug group to the antibody via an R¹ group (where R¹ is an amide). For illustrative purposes only Scheme 9 shows the antibody having four amine groups.

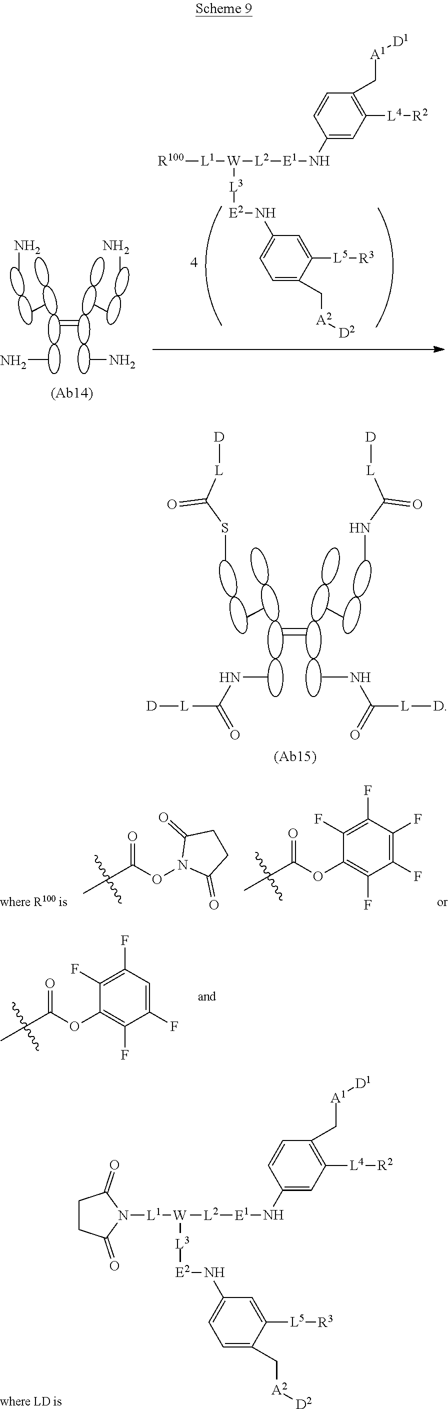

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 10 illustrates this approach for the formation of Antibody Drug Conjugates of Formula (Ab18).

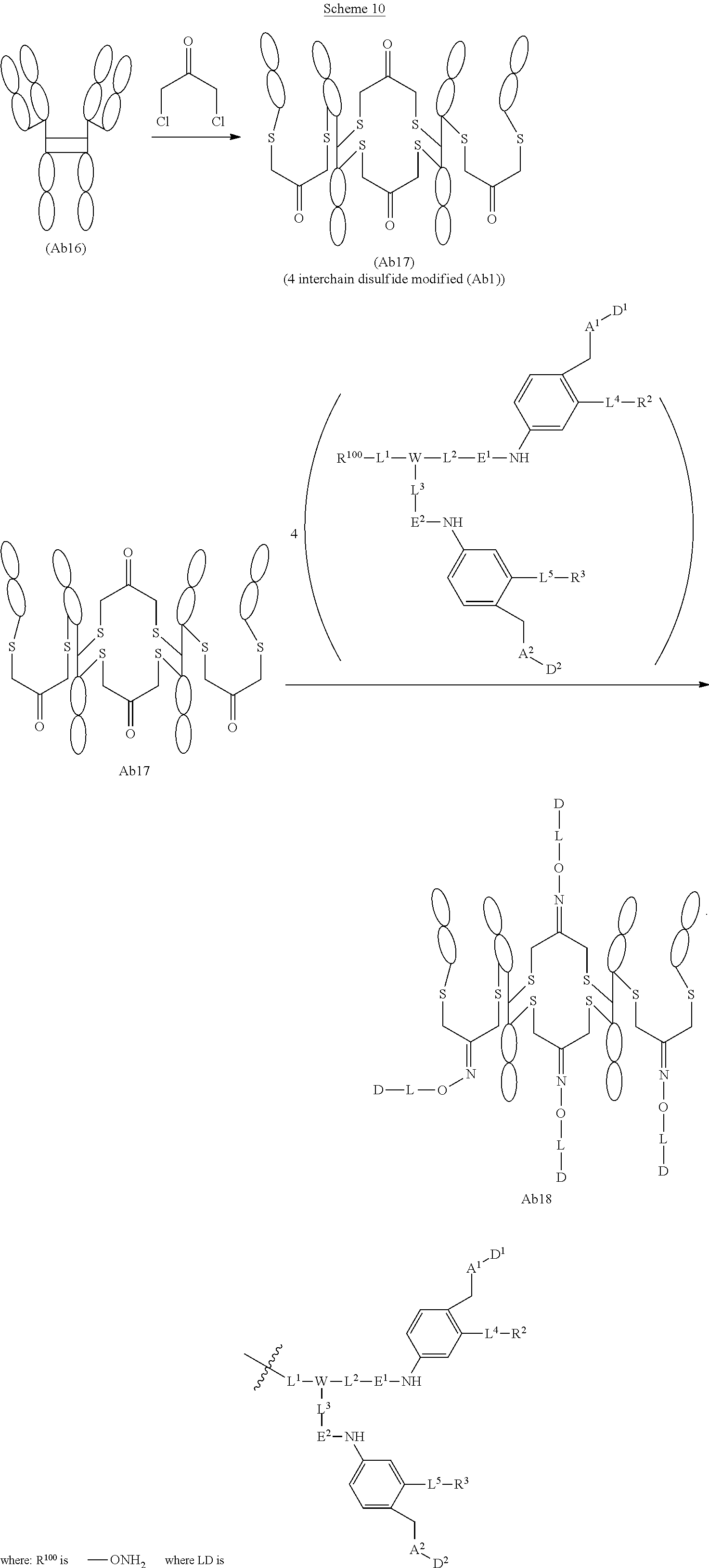

Provided are also protocols for some aspects of analytical methodology for evaluating antibody conjugates of the invention. Such analytical methodology and results can demonstrate that the conjugates have favorable properties, for example properties that would make them easier to manufacture, easier to administer to patients, more efficacious, and/or potentially safer for patients. One example is the determination of molecular size by size exclusion chromatography (SEC) wherein the amount of desired antibody species in a sample is determined relative to the amount of high molecular weight contaminants (e.g., dimer, multimer, or aggregated antibody) or low molecular weight contaminants (e.g., antibody fragments, degradation products, or individual antibody chains) present in the sample. In general, it is desirable to have higher amounts of monomer and lower amounts of, for example, aggregated antibody due to the impact of, for example, aggregates on other properties of the antibody sample such as but not limited to clearance rate, immunogenicity, and toxicity. A further example is the determination of the hydrophobicity by hydrophobic interaction chromatography (HIC) wherein the hydrophobicity of a sample is assessed relative to a set of standard antibodies of known properties. In general, it is desirable to have low hydrophobicity due to the impact of hydrophobicity on other properties of the antibody sample such as but not limited to aggregation, aggregation over time, adherence to surfaces, hepatotoxicity, clearance rates, and pharmacokinetic exposure. See Damle, N.K., Nat Biotechnol. 2008; 26(8):884-885; Singh, S.K., Pharm Res. 2015; 32(11):3541-71. When measured by hydrophobic interaction chromatography, higher hydrophobicity index scores (i.e. elution from HIC column faster) reflect lower hydrophobicity of the conjugates. As shown in Examples below, a majority of the tested antibody conjugates showed a hydrophobicity index of greater than 0.8. In some embodiments, provided are antibody conjugates having a hydrophobicity index of 0.8 or greater, as determined by hydrophobic interaction chromatography.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Abbreviations

AcOH acid acetic
aq. aqueous
Boc tert-butyloxycarbonyl
Boc₂O di-tert-butyl dicarbonate
DCC: dicyclohexylcarbodiimide
DCE: dichloroethane
DCM dichloromethane
DIEA/DIPEA N,N-Diisopropylethylamine
DIAD diisopropylazodicarboxylate
DMAP 4-dimethylaminopyridine
DMTMM 4-(4,6-dimethoxy-1,3,5-triazin-2-yl)-4-methyl-morpholinium
chloride
DMF dimethyl formamide
DMSO dimethyl sulfoxide
EDC/EDC—HCl 1-Ethyl-3-(3-dimethylaminopropyl)carbodiimide hydrochloride
EtOAc(AcOEt) ethyl acetate
EtOH ethanol
FMOC fluorenylmethoxycarbonyl
HATU 1-[Bis(dimethylamino)methylene]-1H-1,2,3-triazolo[4,5-b]pyridinium 3-oxide hexafluorophosphate
HBTU (2-(1H-benzotriazol-1-yl)-1,1,3,3-tetramethyluronium hexafluorophosphate
HOAt 1-Hydroxy-7-azabenzotriazole
HFIP hexafluoroisopropanol
IPA, i-PrOH or 2-PrOH isopropyl alcohol
JosiPhos Pd G3 {(R)-1-[(Sp)-2-(dicyclohexylphosphino)ferrocenyl]ethyldi-tert-butylphosphine}[2-(2′-amino-1,1′-biphenyl)]palladium(II) methanesulfonate
MeCN acetonitrile
MeOH methanol
MTBE methyl tert-butyl ether or tert-butyl methyl ether
NaOtBu sodium tert-butoxide
NMP N-methylpyrrolidine
TES triethylsilane
TBAF tetrabutyl ammonium fluoride
TBAI tetrabutyl ammonium iodide
TBTU [Bis(dimethylamino)methylene]-1H-benzotriazolium 3—Oxide Tetrafluoroborate
Pd(AtaPhos)₂Cl₂ bis(di-tert-butyl(4-dimethylaminophenyl)phosphine) dichloropalladium(II)
PdCl₂(PPh₃)₂ dichlorobis(triphenylphosphine)palladium(II)
Pd₂(dba)₃ tris(dibenzylideneacetone)dipalladium(0)
PMB para-methoxybenzyl
pTsOH para-toluene sulfonic acid
PyBOP benzotriazol-1-yloxytripyrrolidinophosphonium hexafluorophosphate
TSTU O—(N-Succinimidyl)-N,N,N′,N′-tetramethyluronium tetrafluoroborate
THF tetrahydrofuran
TFA trifluoroacetic acid
TFE 2,2,2-Trifluoroethanol
TBAF tetrabutylammonium fluoride
TBDMSCl tert-butyldimethylsilyl chloride
TEA trimethylamine
rt room temperature
sat. saturated
Xantphos 4,5-bis(diphenylphosphino)-9,9-dimethylxanthene

### Example 1. Synthesis and Characterization of Payload and Precursors Thereof

Exemplary payloads and precursors thereof were synthesized using exemplary methods described in this example.

a. Material, Methods & General Procedure for the preparation of the payloads P2-P6:

All reagents obtained from commercial sources were used without further purification. Anhydrous solvents were obtained from commercial sources and used without further drying.

Payload P1 was prepared according to the method described in Example 30 of International PCT publication WO2015/097123. Below is a table showing structures of payloads P1-P8. In the present disclosure, payloads P1-P6 are BH3 mimetics, which correspond to Mcl-1 inhibitor D1-1, Bcl-2 inhibitors D3-1, D3-2, D3-3, and Bcl-xL inhibitors D2-25 and D2-1, respectively. P7 and P8 are antineoplastic non-BH3 mimetics, which correspond to the topoisomerase 1 inhibitors D4-1 and D4-2.

| Payload | BH3 mimetic | Structure |
|---|---|---|
| P1 | D1-1 | 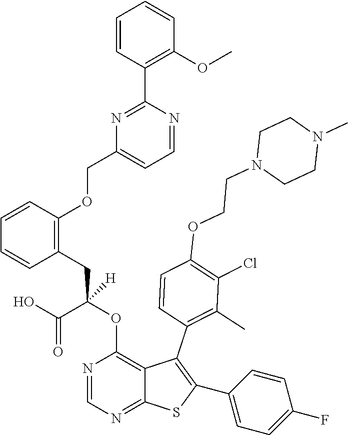 [SMILES: COc1ccccc1-c1nccc(COc2ccccc2C[C@@H](Oc2ncnc3sc(-c4ccc(F)cc4)c(-c4ccc(OCCN5CCN(C)CC5)c(Cl)c4C)c23)C(=O)O)n1] |
| P2 | D3-1 | 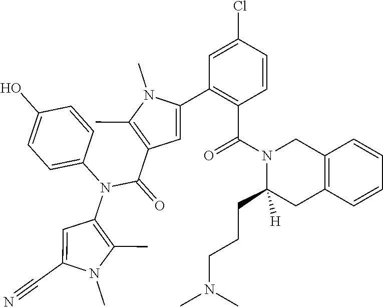 [SMILES: Cc1c(N(C(=O)c2cc(-c3cc(Cl)ccc3C(=O)N3Cc4ccccc4C[C@H]3CCCN(C)C)n(C)c2C)c2ccc(O)cc2)cc(C#N)n1C] |
| P3 | D3-2 | 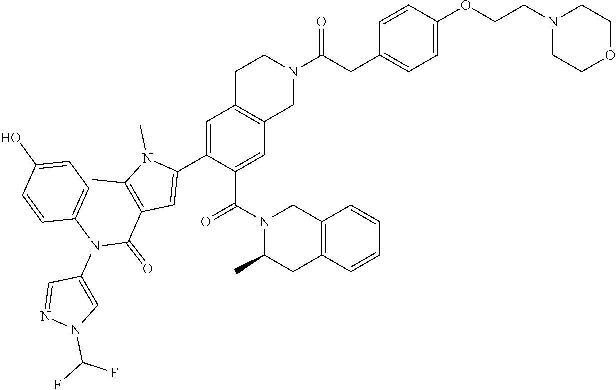 [SMILES: Cc1c(C(=O)N(c2ccc(O)cc2)c2cnn(C(F)F)c2)cc(-c2cc3c(cc2C(=O)N2Cc4ccccc4C[C@H]2C)CN(C(=O)Cc2ccc(OCCN4CCOCC4)cc2)CC3)n1C] |
| P4 | D3-3 | 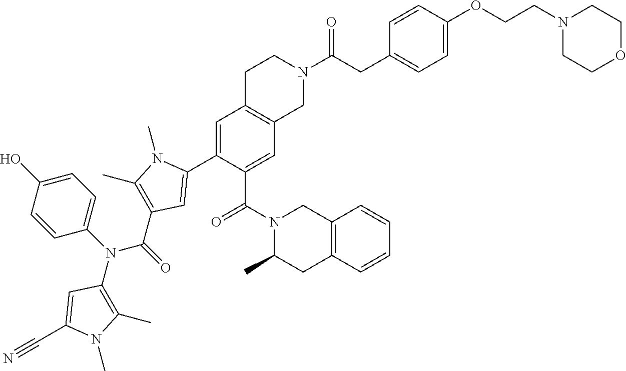 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(O)cc2)cc(C#N)n1C] |
| P5 (also corresponds to P25 described in PCT/ US2021/ 060620) | D2-25 | 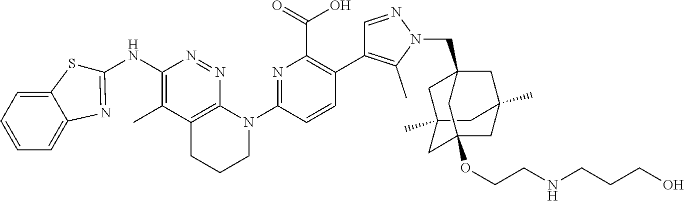 [SMILES: Cc1c(Nc2nc3ccccc3s2)nnc2c1CCCN2c1ccc(-c2cnn(C[C@]34C[C@]5(C)C[C@](C)(C3)C[C@@](OCCNCCCO)(C5)C4)c2C)c(C(=O)O)n1] |
| P6 | D2-1 | 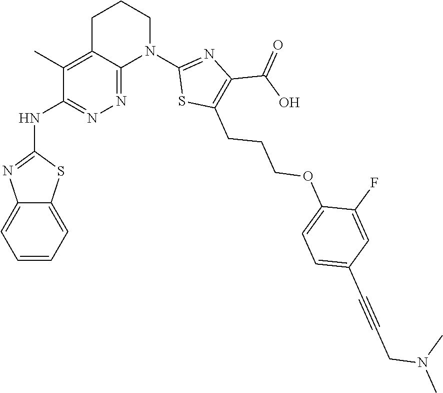 [SMILES: Cc1c(Nc2nc3ccccc3s2)nnc2c1CCCN2c1nc(C(=O)O)c(CCCOc2ccc(C#CCN(C)C)cc2F)s1] |
| | Topomiso- | |
| | merase | |
| Payload | Inhibitors | Structure |
| P7 | D4-1 | |
| P8 | D4-2 | |

### Column Chromatography

Automated flash column chromatography was performed on ISCO CombiFlash® Rf 200 or CombiFlash® Rf+Lumen™ using RediSep* Rf Normal-phase Silica Flash Columns (35-70 pm, 60 A), RediSep Rf Gold® Normal-phase Silica High Performance Columns (20-40 pm, 60 A), RediSep® Rf Reversed-phase C18 Columns (40-63 pm, 60 A), or RediSep Rf Gold® Reversed-phase C18 High Performance Columns (20-40 pm, 100 A).

### TLC

Thin layer chromatography was conducted with 5×10 cm plates coated with Merck Type 60 F₂₅₄ silica-gel.

### Microwave Reactions

Microwave heating was performed with a CEM Discover® SP, or with an Anton Paar Monowave Microwave Reactor.

### NMR

1H-NMR measurements were performed on a Bruker Avance III 500 MHz spectrometer, a Bruker Avance III 400 MHz spectrometer, or a Bruker DPX-400 spectrometer using DMSO-d₆ or CDCl₃ as solvent. 1H NMR data is in the form of delta values, given in part per million (ppm), using the residual peak of the solvent (2.50 ppm for DMSO-d₆ and 7.26 ppm for CDCl₃) as internal standard. Splitting patterns are designated as: s (singlet), d (doublet), t (triplet), q (quartet), quint (quintet), sept (septet), m (multiplet), br s (broad singlet), dd (doublet of doublets), td (triplet of doublets), dt (doublet of triplets), ddd (doublet of doublet of doublets).

### Analytical LC-MS

Certain compounds of the present invention were characterized by high performance liquid chromatography-mass spectroscopy (HPLC-MS) on Agilent HP1200 with Agilent 6140 quadrupole LC/MS, operating in positive or negative ion electrospray ionisation mode. Molecular weight scan range is 100 to 1350. Parallel UV detection was done at 210 nm and 254 nm. Samples were supplied as a 1 mM solution in ACN, or in THF/H₂O (1:1) with 5 μL loop injection. LCMS analyses were performed on two instruments, one of which was operated with basic, and the other with acidic eluents.

Basic LCMS: Gemini-NX, 3 μm, C18, 50 mm×3.00 mm i.d. column at 23° C., at a flow rate of 1 mL min-1 using 5 mM ammonium bicarbonate (Solvent A) and acetonitrile (Solvent B) with a gradient starting from 100% Solvent A and finishing at 100% Solvent B over various/certain duration of time.

Acidic LCMS: KINATEX XB-C18-100A, 2.6 Qm, 50 mm*2.1 mm column at 40° C., at a flow rate of 1 mL min-1 using 0.02% v/v aqueous formic acid (Solvent A) and 0.02% v/v formic acid in acetonitrile (Solvent B) with a gradient starting from 100% Solvent A and finishing at 100% Solvent B over various/certain duration of time.

Certain other compounds of the present invention were characterized HPLC-MS under specific named methods as follows. For all of these methods UV detection was by diode array detector at 230, 254, and 270 nm. Sample injection volume was 1 QL. Gradient elutions were run by defining flow rates and percentage mixtures of the following mobile phases, using HPLC-grade solvents:
Solvent A: 10 mM aqueous ammonium formate+0.04% (v/v) formic acid
Solvent B: Acetonitrile+5.3% (v/v) Solvent A+0.04% (v/v) formic acid.

Retention times (RT) for these named methods are reported in minutes. Ionisation is recorded in positive mode, negative mode, or positive-negative switching mode. Specific details for individual methods follow.

LCMS—V-B methods

Using an Agilent 1200 SL series instrument linked to an Agilent MSD 6140 single quadrupole with an ESI-APCI multimode source (Methods LCMS—V-B1 and LCMS—V-B2) or using an Agilent 1290 Infinity II series instrument connected to an Agilent TOF 6230 with an ESI-jet stream source (Method LCMS—V-B1); column: Thermo Accucore 2.6 μm, C18, 50 mm×2.1 mm at 55° C. Gradient details for methods LCMS—V-B1 and LCMS—V-B2:

| | |
|---|---|
| LCMS-V-B1 | LCMS-V-B2 |

| Time | Solvent | Solvent | Solvent | Solvent | Flow |
|---|---|---|---|---|---|
| (min) | A (%) | B (%) | A (%) | B (%) | (mL/min) |
| 0 | 95 | 5 | 60 | 40 | 1.1 |
| 0.12 | 95 | 5 | 60 | 40 | 1.3 |
| 1.30 | 5 | 95 | 2 | 98 | 1.3 |
| 1.35 | 5 | 95 | 2 | 98 | 1.6 |
| 1.85 | 5 | 95 | 2 | 98 | 1.6 |
| 1.90 | 5 | 95 | 2 | 98 | 1.3 |
| 1.95 | 95 | 5 | 95 | 5 | 1.3 |

LCMS—V-C method

Using an Agilent 1200 SL series instrument linked to an Agilent MSD 6140 single quadrupole with an ESI-APCI multimode source; column: Agilent Zorbax Eclipse plus 3.5 μm, C18(2), 30 mm×2.1 mm at 35° C. Gradient details for method LCMS—V-C:

| Time | Solvent | Solvent | Flow |
|---|---|---|---|
| (min) | A (%) | B (%) | (mL/min) |
| 0 | 95 | 5 | 1 |
| 0.25 | 95 | 5 | 1 |
| 2.50 | 95 | 5 | 1 |
| 2.55 | 5 | 95 | 1.7 |
| 3.60 | 5 | 95 | 1.7 |
| 3.65 | 5 | 95 | 1 |
| 3.70 | 95 | 5 | 1 |
| 3.75 | 95 | 5 | 1 |

### Preparative HPLC

Certain compounds of the present invention were purified by high performance liquid chromatography (HPLC) on an Armen Spot Liquid Chromatography or Teledyne EZ system with a Gemini-NX® 10 μM C18, 250 mm×50 mm i.d. column running at a flow rate of 118 mL min-1 with UV diode array detection (210-400 nm) using 25 mM aqueous NH₄HCO₃ solution and MeCN or 0.1% TFA in water and MeCN as eluents.

Certain other compounds of the present invention were purified by HPLC under specific named methods as follows:

HPLC-V-A methods

These were performed on a Waters FractionLynx MS autopurification system, with a Gemini® 5 μm C18(2), 100 mm×20 mm i.d. column from Phenomenex, running at a flow rate of 20 cm³ min with UV diode array detection (210-400 nm) and mass-directed collection. The mass spectrometer was a Waters Micromass ZQ2000 spectrometer, operating in positive or negative ion electrospray ionisation modes, with a molecular weight scan range of 150 to 1000.

### Method HPLC-V-A1 (pH 4):

Solvent A: 10 mM aqueous ammonium acetate+0.08% (v/v) formic acid; Solvent B: acetonitrile+5% (v/v) Solvent A+0.08% (v/v) formic acid

### Method HPLC-V-A2 (pH 9):

Solvent A: 10 mM aqueous ammonium acetate+0.08% (v/v) conc. ammonia; Solvent B: acetonitrile+5% (v/v) Solvent A+0.08% (v/v) conc. ammonia

HPLC—V-B methods

Performed on an AccQPrep HP125 (Teledyne ISCO) system, with a Gemini® NX 5 μm C18(2), 150 mm×21.2 mm i.d. column from Phenomenex, running at a flow rate of 20 cm³ min with UV (214 and 254 nm) and ELS detection.

### Method HPLC—V-B1 (pH 4):

Solvent A: water+0.08% (v/v) formic acid; solvent B: acetonitrile+0.08% (v/v) formic acid.

### Method HPLC—V-B2 (pH 9):

Solvent A: water+0.08% (v/v) conc. ammonia; solvent B: acetonitrile+0.08% (v/v) conc. ammonia.

Method HPLC—V-B₃ (neutral):

Solvent A: water; Solvent B: acetonitrile.

### Analytical GC-MS

Combination gas chromatography and low resolution mass spectrometry (GC-MS) was performed on Agilent 6850 gas chromatograph and Agilent 5975C mass spectrometer using 15 m×0.25 mm column with 0.25 μm HP-5MS coating and helium as carrier gas. Ion source: EI+, 70 eV, 230° C., quadrupole: 150° C., interface: 300° C.

### High-resolution MS

High-resolution mass spectra were acquired on an Agilent 6230 time-of-flight mass spectrometer equipped with a Jet Stream electrospray ion source in positive ion mode. Injections of 0.5 1 were directed to the mass spectrometer at a flow rate 1.5 ml/min (5 mM ammonium-formate in water and acetonitrile gradient program), using an Agilent 1290 Infinity HPLC system. Jet Stream parameters: drying gas (N2) flow and temperature: 8.0 1/min and 325° C., respectively; nebulizer gas (N2) pressure: 30 psi; capillary voltage: 3000 V; sheath gas flow and temperature: 325° C. and 10.0 1/min; TOFMS parameters: fragmentor voltage: 100 V; skimmer potential: 60 V; OCT 1 RF Vpp:750 V. Full-scan mass spectra were acquired over the m/z range 105-1700 at an acquisition rate of 995.6 ms/spectrum and processed by Agilent MassHunter B.04.00 software.

Chemical naming

IUPAC-preferred names were generated using ChemAxon's ‘Structure to Name’ (s2n) functionality within MarvinSketch or JChemfor Excel (JChem versions 16.6.13-18.22.3), or with the chemical naming functionality provided by Biovia® Draw 4.2.

### Preparation of Bcl-xL Payloads

### General Procedures

### Mitsunobu General Procedure I

To the mixture of 1 eq. of aliphatic alcohol, 1 eq. of carbamate/phenol, and 1 eq. triphenylphosphine in toluene (5 mL/mmol) was added 1 eq. of di-tert-butyl azodicarboxylate. The mixture was stirred at 50° C. for the carbamate and at rt for the phenol. After reaching an appropriate conversion the volatiles were removed under reduced pressure, the crude intermediate was purified via flash chromatography using heptane/EtOAc as eluents.

Deprotection with HFIP General Procedure

Substrate in HFIP (10 mL/mmol) was kept at 100-120° C. in a pressure bottle. After reaching an appropriate conversion the volatiles were removed under reduced pressure, the crude intermediate was purified via flash chromatography using heptane/EtOAc as eluents.

### Sonogashira General Procedure

The mixture of 1 eq. of aryl halogenide, 2 eq. of acetylene, 0.05 eq. of Pd(PPh₃)₂Cl₂, 0.05 eq. of CuI, and DIPA (1 mL/mmol) in THF (5 mL/mmol) was kept at 60° C. After reaching an appropriate conversion the volatiles were removed under reduced pressure, the crude intermediate was purified via flash chromatography using heptane/EtOAc as eluents.

### Buchwald General Procedure II

The mixture of chloro compound, 2 eq. of 1,3-benzothiazol-2-amine, 10 mol % of JosiPhos Pd (G3) and 3 eq. of DIPEA suspended in 1,4-dioxane (5 mL/mmol) were stirred at reflux until no further conversion was observed. Celite was added to the reaction mixture and the volatiles were removed under reduced pressure. Then it was purified via flash chromatography on 120 g silica gel column using heptane-EtOAc or DCM-MeOH (1.2% NH₃) as eluents.

Propargylic amine preparation General Procedure

An oven-dried vial was equipped with a PTFE-coated magnetic stirring bar, it was charged with 2 eq. PPh3 and 2 eq. imidazole then DCM (5 mL/mmol) was added. To the resulting mixture 2 eq. iodine was added portionwise then stirred for 15 min at rat. To the resulting mixture 1 eq. of the appropriate alcohol was added dissolved in DCM and stirred at rt until no further conversion was observed. To the generated iodo compound 20 eq. of the appropriate amine was added and then stirred for 30 min at rt, while full conversion was observed. Celite was added to the reaction mixture and the volatiles were removed under reduced pressure. Then it was purified via flash chromatography using DCM and MeOH (1.2% NH₃) eluents.

### Hydrolysis General Procedure

The appropriate methyl ester was suspended in a 1:1 mixture of THF - water (5 mL/mmol) and 10 eq. of LiOH x H₂O was added, and the mixture was stirred at 50° C. After reaching an appropriate conversion, the volatiles were removed under reduced pressure; the crude product was purified via flash chromatography using DCM and MeOH (containing 1.2% NH₃) as eluents.

Preparation A: 3-(3,6-dichloro-5-methyl-pyridazin-4-yl)propan-1-ol

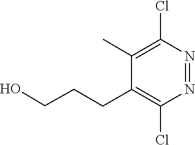

### Step A: [(pent-4-yn-1-yloxy)methyl]benzene

To an oven-dried flask was added 4-pentyn-1-ol (11.1 mL, 119 mmol, 1 eq) in THF (100 mL) and the solution was cooled to 0° C. Sodium hydride (60% dispersion; 7.13 g, 1.5 eq) was added portionwise and the mixture was allowed to stir for 30 min at 0° C. before the dropwise addition of benzyl bromide (15.6 mL, 131 mmol, 1.1 eq). The mixture was allowed to warm to ambient temperature and was stirred for 16 h, then cooled to 0° C., quenched with saturated aqueous ammonium chloride (30 mL) and diluted with water (30 mL). The mixture was extracted with ethyl acetate (2×150 mL), and the combined organic extracts were washed successively with dilute aqueous ammonium hydroxide (150 mL) and brine (100 mL), dried (magnesium sulfate) and concentrated in vacuo. Purification by automated flash column chromatography (CombiFlash Rf, 330 g RediSep™ silica cartridge) eluting with a gradient of ethyl acetate in iso-heptane afforded the desired product (19.5 g, 94%). ¹H NMR (400 MHz, Chloroform-d) δ 7.37-7.32 (m, 4H), 7.31-7.27 (m, 1H), 4.52 (s, 2H), 3.58 (t, J=6.1 Hz, 2H), 2.32 (td, J=7.1, 2.6 Hz, 2H), 1.95 (t, J=2.7 Hz, 1H), 1.83 (tt, J=7.1, 6.2 Hz, 2H); LC/MS (C₁₂H₁₄O) 175 [M+H]⁺.

### Step B: [(hex-4-yn-1-yloxy)methyl]benzene

To an oven-dried flask was added the product from Step A (19.5 g, 112 mmol, 1 eq) and tetrahydrofuran (200 mL) and the solution was cooled to −78° C. n-Butyllithium (2M solution in hexanes, 66.9 mL, 135 mmol, 1.2 eq) was added dropwise over 30 min and the reaction was stirred for 1 h then iodomethane (10.5 mL, 168 mmol, 1.5 eq) was added dropwise and the mixture was allowed to warm to 0° C. over 1 h. The reaction was quenched by the addition of saturated aqueous ammonium chloride (40 mL), diluted with water (40 mL), extracted with ethyl acetate (3×100 mL), and the combined organic extracts were successively washed with 2M aqueous sodium thiosulfate (200 mL) and brine (200 mL), dried (magnesium sulfate) and concentrated in vacuo. Purification by automated flash column chromatography (CombiFlash Rf, 330 g RediSep™ silica cartridge) eluting with a gradient of 0-10% ethyl acetate in iso-heptane afforded the desired product (19.2 g, 91%). ¹H NMR (400 MHz, DMSO-d6) δ 7.41-7.23 (m, 5H), 4.46 (s, 2H), 3.48 (t, J=6.3 Hz, 2H), 2.23-2.14 (m, 2H), 1.72 (s, 3H), 1.70-1.65 (m, 2H); LC/MS (C₁₃H₁₆O) 189 [M+H]⁺.

### Step C: 4-[3-(benzyloxy)propyl]-3,6-dichloro-5-methylpyridazine

A solution of 3,6-dichloro-1,2,4,5-tetrazine (5 g, 33.1 mmol, 1 eq) and the product from Step B (7.48 g, 39.8 mmol, 1.2 eq) in tetrahydrofuran (30 mL) was heated at 160° C. for 19 h in a sealed flask. The reaction was allowed to cool to ambient temperature then concentrated in vacuo. Purification by automated flash column chromatography (CombiFlash Rf, 220 g RediSep™ silica cartridge) eluting with a gradient of 0-30% ethyl acetate in iso-heptane afforded the desired product (7.32 g, 71%). ¹H NMR (400 MHz, DMSO-d6) δ 7.45-7.18 (m, 5H), 4.48 (s, 2H), 3.53 (t, J=5.9 Hz, 2H), 2.96-2.83 (m, 2H), 2.42 (s, 3H), 1.88-1.69 (m, 2H); LC/MS (C₁₅H₁₆Cl₂N₂O) 311 [M+H]⁺.

### Step D: 3-(3,6-dichloro-5-methyl-pyridazin-4-yl)propan-l-ol

To a cooled solution of the product from Step C (7.32 g, 23.5 mmol, 1 eq) in dichloromethane (100 mL) was added boron trichloride solution (1 M in dichloromethane; 58.8 mL, 58.8 mmol, 2.5 eq) dropwise and the mixture was allowed to stir at ambient temperature for 1 h. The reaction was quenched by the addition of methanol and concentrated in vacuo. The residue was partitioned between dichloromethane (100 mL) and saturated aqueous sodium bicarbonate (150 mL), and the organic phase was washed with brine (150 mL), dried (magnesium sulfate) and concentrated in vacuo. Purification by automated flash column chromatography (CombiFlash Rf, 80 g RediSep™ silica cartridge) eluting with a gradient of 0-80% ethyl acetate in iso-heptane afforded the desired product (4.19 g, 81%). ¹H NMR (400 MHz, DMSO-d₆) δ 4.67 (t, J=5.1 Hz, 1H), 3.49 (td, J=6.0, 5.1 Hz, 2H), 2.91-2.80 (m, 2H), 2.43 (s, 3H), 1.72-1.59 (m, 2H); LC/MS (C₈H₁₀Cl₂N₂O) 221 [M+H]⁺.

### [679]Preparation B: 3,6-dichloro-4-(3-iodopropyl)-5-methyl-pyridazine

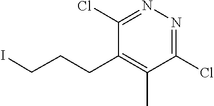

After stirring PPh₃ (59.3 g, 2 eq), imidazole (15.4 g, 2 eq), and iodine (57.4 g, 2 eq) in 560 mL of DCM for 15 min, 25.0 g of Preparation A (113 mmol) was added and stirred for 2 h. The product was purified via flash chromatography using heptane and EtOAc as eluents to give 34.7 g of the desired product (92%). ¹H NMR (500 MHz, DMSO-d₆) δ ppm 3.41 (t, 2H), 2.89 (m, 2H), 2.43 (s, 3H), 1.97 (m, 2H); ¹³C NMR (125 MHz, DMSO-d₆) δ ppm 157.7, 156.8, 141.5, 140.2, 31.4, 31.1, 16.7, 7.8; HRMS (ESI) [M]⁺calcd for C₈H₉Cl₂IN₂: 330.9266, found 330.9255.

### Preparation C: tert-butyl-diphenyl-[2-[[3,5-dimethyl-7-[[5-methyl-4-(4,4,5,5-tetramethyl-1,3,2-dioxaborolan-2-yl)pyrazol-1-yl]methyl]-1-adamantyl]oxy]ethoxy]silane

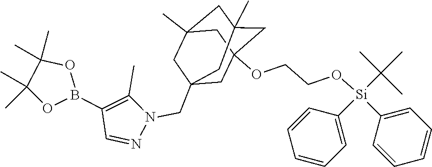 [SMILES: Cc1c(B2OC(C)(C)C(C)(C)O2)cnn1CC12CC3(C)CC(C)(C1)CC(OCCO[Si](c1ccccc1)(c1ccccc1)C(C)(C)C)(C3)C2]

### [682]Step A: 3-bromo-5,7-dimethyladamantane-1-carboxylic acid

After stirring iron (6.7 g, 120 mmol) in bromine (30.7 mL, 600 mmol, 5 eq) at 0° C. for 1 h, 3,5-dimethyladamantane-1-carboxylic acid (25 g, 1 eq) was added and the reaction mixture was stirred at rt for 2 days. After the addition of EtOAc, the reaction mixture was treated carefully with a saturated solution of sodium-thiosulfate at 0° C. and stirred for 15 min. After filtration through a pad of Celite and rinsing with EtOAc, the organic phase was separated, washed with a saturated solution of sodium-thiosulfate and brine, dried, concentrated to give the desired product (34.28 g, 74.6%), which was used without further purification. ¹H NMR (400 MHz, DMSO-d₆): δ ppm 12.33 (br., 1H), 2.21 (s, 2H), 1.96/1.91 (d+d, 4H), 1.50/1.43 (d+d, 4H), 1.21/1.14 (dm+dm, 2H), 0.86 (s, 6H); ¹³C NMR (100 MHz, DMSO-d₆) δ ppm 176.8, 66.8, 54.0, 48.7, 48.5, 45.7, 43.3, 35.5, 29.4; HRMS-ESI (m/z): [M-H]- calcd for C13HisBrO₂: 285.0496; found 285.0498.

### Step B: 3-bromo-5,7-dimethyl-1-adamantyl-methanol

To the product from Step A (34.3 g, 119 mmol) in THF (77.6 mL) was added slowly a 1 M solution of BH₃-THF in THF (358 mL, 3 eq) and the reaction mixture was stirred for 18 h. After the addition of methanol and stirring for 30 min, purification by column chromatography (silica gel, heptane and MTBE as eluents) afforded the desired product (16.19 g, 49.6%). ¹H NMR (400 MHz, DMSO-d₆): δ ppm 4.51 (t, 1H), 3.05 (d, 2H), 1.91 (s, 2H), 1.91 (s, 4H), 1.19/1.09 (d+d, 2H), 1.19/1.05 (d+d, 4H), 0.85 (s, 6H)¹³C NMR (100 MHz, DMSO-d₆) δ ppm 70.4, 68.9, 54.9, 49.8, 49.3, 43.8, 41.4, 35.7, 29.7; HRMS-ESI (m/z): [M-Br]- calcd for Cl₃H210: 193.1598 found: 193.1589.

### Step C: 1-[3-bromo-5,7-dimethyl-1-adamantyl]methyl]pyrazole

To the product from Step B (16.19 g, 59.26 mmol) and 1H-pyrazole (4.841 g, 1.2 eq) in toluene (178 mL) was added cyanomethylenetributylphosphorane (18.64 mL, 1.2 eq) in one portion and the reaction mixture was stirred at 90° C. for 2 h. Purification by column chromatography (silica gel, heptane and MTBE as eluents) afforded the desired product (17.88 g, 93%). ¹H NMR (400 MHz, DMSO-d₆): δ ppm 7.63 (d, 1H), 7.43 (d, 1H), 6.23 (t, 1H), 3.90 (s, 2H), 1.92-1.02 (m, 12H), 0.83 (s, 6H); ¹³C NMR (100 MHz, DMSO-d₆) δ ppm 139.0, 131.8, 105.2, 67.7, 61.4, 54.4/48.8/44.6, 50.4, 35.7, 29.6; HRMS-ESI (m/z): [M]⁺calcd for C₁₆H₂₃BrN₂: 322.1045 found: 322.1014.

### Step D: 5-methyl-]-[[-3-bromo-5,7-dimethyl-1-adamantyl]methyl]pyrazole

To the solution of the product from Step C (17.88 g, 55.3 mmol) in THF (277 mL) was added butyllithium (2.5 M in THF, 66 mL, 3 eq) at −78° C., then after 1 h, iodomethane (17.2 mL, 5 eq) was added. After 10 min, the reaction mixture was quenched with a saturated solution of NH₄C₁, extracted with EtOAc and the combined organic layers were dried and concentrated to give the desired product (18.7 g, 100%), which was used in the next step without further purification. ¹H NMR (400 MHz, DMSO—d₆): 6 ppm 7.31 (d, 1H), 6.00 (d, 1H), 3.79 (s, 2H), 2.23 (s, 3H), 2.01 (s, 2H), 1.89/1.85 (d+d, 4H), 1.23/1.15 (d+d, 4H), 1.16/1.05 (d+d, 2H), 0.83 (s, 6H); ¹³C NMR (100 MHz, DMSO-d₆) δ ppm 139.2, 138.0, 105.2, 67.8, 57.8, 54.4, 50.6, 48.8, 44.8, 41.5, 35.7, 29.6, 11.8; HRMS-ESI (m/z): [M+H]⁺calcd for C₁₇H₂₆BrN₂: 337.1279 found: 337.1289.

### Step E: 2-[[-3,5-dimethyl-7-[(5-methylpyrazol-1-yl)methyl]-1-adamantyl]oxy]ethanol

The mixture of the product from Step D (18.7 g, 55.3 mmol), ethylene glycol (123 mL, 40 eq), and DIPEA (48.2 mL, 5 eq) was stirred at 120° C. for 6 h. After the reaction mixture was diluted with water and extracted with EtOAc, the combined organic layers were dried and concentrated to give the desired product (18.5 g, 105%), which was used in the next step without further purification. ¹H NMR (400 MHz, DMSO-d₆): δ ppm 7.29 (d, 1H), 5.99 (d, 1H), 4.45 (t, 1H), 3.78 (s, 2H), 3.39 (q, 2H), 3.32 (t, 2H), 2.23 (s, 3H), 1.34 (s, 2H), 1.27/1.21 (d+d, 4H), 1.13/1.07 (d+d, 4H), 1.04/0.97 (d+d, 2H), 0.84 (s, 6H); ¹³C NMR (100 MHz, DMSO-d₆) δ ppm 139.0, 137.8, 105.1, 74.0, 62.1, 61.5, 58.5, 50.1, 47.0, 46.1, 43.3, 39.7, 33.5, 30.2, 11.9; HRMS-ESI (m/z): [M+H]⁺calcd for C₁₉H₃₁N₂O₂: 319.2386 found: 319.2387.

### Step F: tert-butyl-diphenyl-[2-[[-3,5-dimethyl-7-[(5-methylpyrazol-1-yl)methyl]-1-adamantyl]oxy]ethoxy]silane

To the mixture of the product from Step E (17.6 g, 55.3 mmol) and imidazole (5.65 g, 1.5 eq) in DCM (150 ml) was added tert-butyl-chloro-diphenyl-silane (18.6 g, 1.2 eq) and the reaction mixture was stirred for 1 h. Purification by column chromatography (silica gel, heptane and MTBE as eluents) afforded the desired product (27.0 g, 87.8%). ¹H NMR (400 MHz, DMSO-d₆): δ ppm 7.72-7.34 (m, 10H), 7.29 (d, 1H), 5.99 (br., 1H), 3.78 (s, 2H), 3.67 (t, 2H), 3.44 (t, 2H), 2.21 (s, 3H), 1.33 (s, 2H), 1.26/1.18 (d+d, 4H), 1.12/1.06 (d+d, 4H), 1.03/0.96 (d+d, 2H), 0.98 (s, 9H), 0.82 (s, 6H); ¹³C NMR (100 MHz, DMSO-d₆) δ ppm 139.0, 137.8, 105.1, 74.2, 64.4, 61.7, 58.5, 50.0, 46.9, 46.0, 43.4, 39.6, 33.5, 30.1, 27.1, 19.3, 11.9; HRMS-ESI (m/z): [M+H]⁺calcd for C₃₅H4₉N20₂Si: 557.3563 found: 557.3564.

Step G: tert-butyl-diphenyl-[2-[[3-[(4-iodo-5-methyl-pyrazol-1-yl)methyl]-5,7-dimethyl-1-adamantyl]oxy]ethoxy]silane

To the solution of the product from Step F (27.0 g, 48.56 mmol) in DMF (243 mL) was added N-iodosuccinimide (13.6 g, 1.25 eq) and the reaction mixture was stirred for 2 h. After the dilution with water, the mixture was extracted with DCM. The combined organic layers were washed with saturated solution of sodium-thiosulphate and brine, dried, and concentrated to afford the desired product (30.1 g, 90%). ¹H NMR (400 MHz, DMSO-d₆): δ ppm 7.68-7.37 (m, 10H), 7.45 (s, 1H), 3.89 (s, 2H), 3.67 (t, 2H), 3.44 (t, 2H), 2.23 (s, 3H), 1.30 (s, 2H), 1.26/1.17 (d+d, 4H), 1.12/1.05 (d+d, 4H), 1.00/0.96 (d+d, 2H), 0.98 (s, 9H), 0.82 (s, 6H); ¹³C NMR (100 MHz, DMSO-d₆) δ ppm 142.5, 140.8, 133.7, 64.4, 61.7, 60.3, 59.9, 49.9, 46.8, 45.9, 43.2, 39.7, 33.5, 30.1, 27.1, 19.3, 12.2; HRMS-ESI (m/z): [M+H]⁺calcd for C₃₅H₄₈IN₂O₂Si: 683.2530 found: 683.2533.

### Step H: tert-butyl-diphenyl-[2-[[3,5-dimethyl-7-[[5-methyl-4-(4,4,5,5-tetramethyl-1,3,2-dioxaborolan-2-yl)pyrazol-1-yl]methyl]-1-adamantyl]oxy]ethoxy]silane

To the product from Step G (17.5 g, 25.6 mmol) in THF (128 mL) was added chloro(isopropyl)magnesium-LiCl (1.3 M in THF, 24 mL, 1.2 eq) at 0° C., stirred for 40 min, treated with 2-isopropoxy-4,4,5,5-tetramethyl-1,3,2-dioxaborolane (15.7 mL, 3 eq), and the reaction mixture was stirred for 10 min. After dilution with a saturated solution NH₄Cl and extraction with EtOAc, the combined organic phases were concentrated and was purified by column chromatography (silica gel, heptane and MTBE as eluents) to give the desired product (15.2 g, 86.9%). ¹H NMR (400 MHz, DMSO—d₆): δ ppm 7.65 (dm, 4H), 7.47 (s, 1H), 7.45 (tm, 2H), 7.40 (tm, 4H), 3.80 (s, 2H), 3.66 (t, 2H), 3.44 (t, 2H), 2.35 (s, 3H), 1.35-0.94 (m, 12H), 1.24 (s, 12H), 0.97 (s, 9H), 0.83 (s, 6H); ¹³C NMR (100 MHz, DMSO-d₆) δ ppm 146.9, 144.3, 135.6, 130.2, 128.2, 104.7, 83.0, 74.2, 64.4, 61.7, 58.4, 30.1, 27.1, 25.2, 19.3, 12.0; HRMS-ESI (m/z): [M+H]⁺calcd for C₄₁H₆₀BN₂O₄Si: 683.4415 found: 683.4423.

### Preparation D: methyl 3-bromo-6-[3-(3,6-dichloro-5-methyl-pyridazin-4-yl)propylamino]pyridine-2-carboxylate

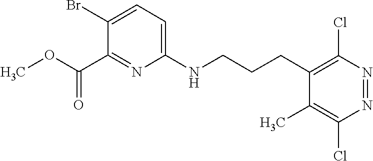 [SMILES: COC(=O)c1nc(NCCCc2c(Cl)nnc(Cl)c2C)ccc1Br]

### [699]Step A: methyl 6-[bis(tert-butoxycarbonyl)amino]-3-bromo-pyridine-2-carboxylate

To methyl 6-amino-3-bromo-pyridine-2-carboxylate (25.0 g, 108.2 mmol) and DMAP (1.3 g, 0.1 eq) in DCM (541 mL) was added Boc₂0 (59.0 g, 2.5 eq) at 0° C. and the reaction mixture was stirred for 2.5 h. After the addition of a saturated solution of NaHCO₃ and extraction with DCM, the combined organic phases were dried and concentrated to afford the desired product (45.0 g, 72.3%). LC/MS (Cl₇H₂₃BrN₂O₆Na) 453 [M+Na]⁺.

### [701]Step B: methyl 3-bromo-6-(tert-butoxycarbonylamino)pyridine-2-carboxylate

To the product from Step A (42.7 g, 74.34 mmol) in DCM (370 mL) was added TFA (17.1 mL, 3 eq) at 0° C. and the reaction mixture was stirred for 18 h. After washing with a saturated solution of NaHCO₃ and brine, the combined organic phases were dried, concentrated, and purified by column chromatography (silica gel, heptane and EtOAc as eluents) to give the desired product (28.3 g, 115.2%).

¹H NMR (400 MHz, DMSO-d₆): δ ppm 10.29 (s, 1H), 8.11 (d, 1H), 7.88 (d, 1H), 3.87 (s, 3H), 1.46 (s, 9H)¹³C NMR (100 MHz, DMSO-d₆) δ ppm 165.6, 153.1, 151.8/148.3, 143.5, 116.3, 109.2, 53.2, 28.4. LC/MS (Cl₂H₁₅BrN₂O₄Na) 353 [M+Na]⁺.

### Step C: methyl 3-bromo-6-[tert-butoxycarbonyl-[3-(3,6-dichloro-5-methyl-pyridazin-4-yl)propyl]amino]pyridine-2-carboxylate

To the product from Step B (10.0 g, 30.1967 mmol) in acetone (150 mL), were added Cs₂CO₃ (29.5 g, 3 eq) and Preparation B (9.9 g, 1 eq) and the reaction mixture was stirred for 18 h. After dilution with water and extraction with EtOAc, the combined organic phases were washed with brine, dried and concentrated to give the desired product (17.5 g, 108%). ¹H NMR (400 MHz, DMSO-d₆): δ ppm 8.13 (d, 1H), 7.78 (d, 1H), 3.91 (t, 2H), 3.89 (s, 3H), 2.79 (m, 2H), 2.38 (s, 3H), 1.82 (m, 2H), 1.46 (s, 9H); ¹³C NMR (100 MHz, DMSO-d₆) δ ppm 165.3, 157.6, 156.6, 153.2, 152.9, 147.2, 143.1, 142.2, 139.7, 122.6, 111.8, 82.2, 53.3, 46.4, 28.1, 27.7, 26.5, 16.3; HRMS-ESI (m/z): [M+Na]⁺calcd for C₂₀H₂₃BrCl₂N₄NaO₄: 555.0177 found: 555.0172.

### Step D: methyl 3-bromo-6-[3-(3,6-dichloro-5-methyl-pyridazin-4-yl)propylamino]pyridine-2-carboxylate

The product from Step C (17.5 g, 32.7 mmol) in 1,1,1,3,3,3-hexafluoroisopropanol (330 mL) was stirred at 110° C. for 18 h. Purification by column chromatography (silica gel, heptane and EtOAc as eluents) afforded the desired product (9.9 g, 70%). ¹H NMR (400 MHz, DMSO-d₆): δ ppm 7.63 (d, 1H), 7.22 (t, 1H), 6.57 (d, 1H), 3.83 (s, 3H), 3.30 (m, 2H), 2.83 (m, 2H), 2.37 (s, 3H), 1.74 (m, 2H)¹³C NMR (100 MHz, DMSO-d₆) δ ppm 166.5, 141.5, 112.6, 52.9, 40.9, 28.0, 27.0, 16.4.

### Preparation E: (4-methoxyphenyl)methyl 3-bromo-6-[3-(3,6-dichloro-5-methyl-pyridazin-4-yl)propylamino]pyridine-2-carboxylate

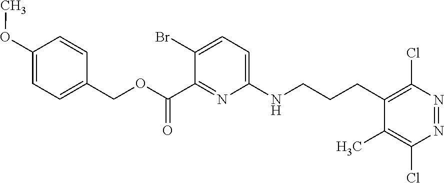 [SMILES: COc1ccc(COC(=O)c2nc(NCCCc3c(Cl)nnc(Cl)c3C)ccc2Br)cc1]

### Step A: 3-bromo-6-[3-(3,6-dichloro-5-methyl-pyridazin-4-yl)propylamino]pyridine-2-carboxylic acid

The mixture of the product from Preparation D (35.39 g, 81.52 mmol) and LiOH×H₂O (13.68 g, 4 eq) in 1,4-dioxane (408 mL) and water (82 mL) was stirred at 60° C. for 1 h. After quenching with a 1 M solution of HCl and extraction with EtOAc, the combined organic phases were dried, concentrated, and purified by flash chromatography (silica gel, using DCM and MeOH as eluents) to give the desired product (27.74 g, 81%). LC/MS (C₁₋₄H₁₄BrCl₂N₄O₂) 421 [M+H]⁺.

### Step B: (4-methoxyphenyl)methyl 3-bromo-6-[3-(3,6-dichloro-5-methyl-pyridazin-4-yl)propylamino]pyridine-2-carboxylate

To the product of Step A (27.7 g, 65.9 mmol), (4-methoxyphenyl)methanol (16.4 mL, 2 eq), and PPh₃ (34.6 g, 2 eq) in toluene (660 mL) and THF (20 ml) was added dropwise diisopropyl azodicarboxylate (26 mL, 2 eq) and the reaction mixture was stirred at 50° C. for 1 h. Purificationby flash chromatography (silica gel, using heptane and EtOAc as eluents) afforded the desired product (23.65 g, 66.4%). ¹H NMR (500 MHz, dmso-d6) 6 ppm 7.62 (d, 1H), 7.37 (dn, 2H), 7.21 (t, 1H), 6.91 (dm, 2H), 6.56 (d, 1H), 5.25 (s, 2H), 3.74 (s, 3H), 3.30 (q, 2H), 2.81 (m, 2H), 2.33 (s, 3H), 1.73 (m, 2H); ¹³C NMR (500 MHz, dmso-d6) 6 ppm 165.9, 159.7, 157.6, 157.5, 156.8, 148.0, 142.7, 141.5, 139.7, 130.6, 127.8, 114.3, 112.6, 101.6, 67.0, 55.6, 40.9, 28.0, 27.1, 16.4; HRMS-ESI (m/z): [M+H]⁺calcd for C₂₂H₂₂BrCl₂N₄O₃: 539.0252, found: 539.0246.

### Preparation F: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(p-tolylsulfonyloxy)ethoxy]-1-adamantyl]methyl]-5-methyl-pyrazol-4-yl]pyridine-2-carboxylate

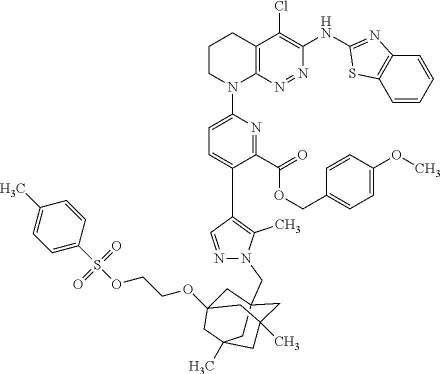 [SMILES: COc1ccc(COC(=O)c2nc(N3CCCc4c3nnc(Nc3nc5ccccc5s3)c4Cl)ccc2-c2cnn(CC34CC5(C)CC(C)(C3)CC(OCCOS(=O)(=O)c3ccc(C)cc3)(C5)C4)c2C)cc1]

### [713]Step A: (4-methoxyphenyl)methyl 3-[ ]-[[3-[2-[tert-butyl(diphenyl)silyl]oxyethoxy]-5,7-dimethyl-1-adamantyl]methyl]-5-methyl-pyrazol-4-yl]-6-[3-(3,6-dichloro-5-methyl-pyridazin-4-yl)propylamino]pyridine-2-carboxylate

The mixture of the product from Preparation E (9.78 g, 18.1 mmol), the product from Preparation C (13.6 g, 1.1 eq), Pd(AtaPhos)₂Cl₂ (801 mg, 0.1 eq), and Cs₂CO₃ (17.7 g, 3 eq) in 1,4-dioxane (109 mL) and H₂O (18 mL) was stirred at 80° C. for 8 h. After quenching the cooled reaction with brine, the mixture was extracted with EtOAc and the combined organic layers were dried and concentrated to give the desired product (21.9 g, 119%), which was used in the next step without further purification. ¹H NMR (400 MHz, DMSO-d₆): δ ppm 7.68-7.35 (m, 10H), 7.31 (d, 1H), 7.27 (s, 1H), 7.11 (dm, 2H), 6.98 (t, 1H), 6.83 (dm, 2H), 6.62 (d, 1H), 4.99 (s, 2H), 3.80 (s, 2H), 3.70 (s, 3H), 3.65 (t, 2H), 3.44 (t, 2H), 3.34 (q, 2H), 2.84 (m, 2H), 2.34 (s, 3H), 2.01 (s, 3H), 1.77 (m, 2H), 1.38-0.89 (m, 12H), 0.97 (s, 9H), 0.82 (s, 6H); ¹³C NMR (500 MHz, dmso-d6) 6 ppm 140.4, 137.6, 130.1, 114.2, 110.3, 66.3, 64.4, 61.7, 59.0, 55.5, 40.9, 30.1, 28.1, 27.3, 27.1, 16.4, 10.8; HRMS-ESI (m/z): [M+H]⁺calcd for Cs₇H₆₉Cl₂N₆OsSi: 1015.4475 found: 1015.4474.

### Step B: (4-methoxyphenyl)methyl 3-[ ]-[[3-[2-[tert-butyl(diphenyl)silyl]oxyethoxy]-5,7-dimethyl-1-adamantyl]methyl]-5-methyl-pyrazol-4-yl]-6-(3-chloro-4-methyl-6,7-dihydro-SH-pyrido[2,3-c]pyridazin-8-yl)pyridine-2-carboxylate

The mixture of the product from Step A (21.9 g, 21.6 mmol), Cs₂CO₃ (14 g, 2 eq), DIPEA (7.5 mL, 2 eq) and Pd(Ataphos)₂Cl₂ (954 mg, 0.1 eq) in 1,4-dioxane (108 mL) was stirred at 110° C. for 18 h. After quenching with water and extracting with EtOAc, the combined organic phases were dried, concentrated, and purified by column chromatography (silica gel, DCM and EtOAc as eluents) to give the desired product (8.4 g, 40%). ¹H NMR (400 MHz, DMSO-d₆): δ ppm 7.84 (d, 1H), 7.67 (d, 1H), 7.65 (d, 4H), 7.44 (t, 2H), 7.41 (s, 1H), 7.40 (t, 4H), 7.15 (d, 2H), 6.87 (d, 2H), 5.07 (s, 2H), 3.96 (t, 2H), 3.83 (s, 2H), 3.71 (s, 3H), 3.66 (t, 2H), 3.45 (t, 2H), 2.86 (t, 2H), 2.29 (s, 3H), 2.08 (s, 3H), 1.97 (qn, 2H), 1.38 (s, 2H), 1.25/1.18 (d+d, 4H), 1.18/1.12 (d+d, 4H), 1.0I/O.93 (d+d, 2H), 0.97 (s, 9H), 0.82 (s, 6H); ¹³C NMR (100 MHz, DMSO-d₆) δ ppm 166.8, 159.7, 156.3, 153.6, 150.8, 147.7, 140.1, 137.6, 137.3, 136.0, 135.6, 133.8, 130.2, 130.2, 129.1, 128.2, 127.7, 123.0, 120.4, 115.6, 114.3, 74.2, 66.8, 64.4, 61.7, 59.3, 55.6, 49.9, 46.8, 46.0, 46.0, 43.3, 39.7, 33.6, 30.1, 27.1, 24.6, 21.0, 19.3, 15.5, 10.8; HRMS-ESI (m/z): [M+H]⁺calcd for C₅₇H6sClN₆O₅Si: 979.4709 found: 979.4710.

### Step C: (4-methoxyphenyl)methyl 6-(3-chloro-4-methyl-6,7-dihydro-5H-pyrido[2,3-c]pyridazin-8-yl)-3-[ ]-[[3-(2-hydroxyethoxy)-5,7-dimethyl-]-adamantyl]methyl]-5-methyl-pyrazol-4-yl]pyridine-2-carboxylate

To the product from Step B (8.4 g, 8.6 mmol) in THF (86 mL) was added a 1 M solution of TBAF in THF (9.4 mL, 1.1 eq) at 0° C. and the reaction mixture was stirred at room temperature for 1.5 h. After quenching with a saturated solution of NH₄C₁ and extracted with EtOAc, the combined organic phases were washed with brine, dried, concentrated, and purified by column chromatography (silica gel, DCM and MeOH as eluents) to give the desired product (4.7 g, 74%). ¹H NMR (400 MHz, DMSO-d₆): δ ppm 7.85 (d, 1H), 7.70 (d, 1H), 7.39 (s, 1H), 7.18 (d, 2H), 6.90 (d, 2H), 5.10 (s, 2H), 4.45 (t, 1H), 3.96 (t, 2H), 3.84 (s, 2H), 3.74 (s, 3H), 3.40 (q, 2H), 3.33 (t, 2H), 2.86 (t, 2H), 2.29 (s, 3H), 2.09 (s, 3H), 1.98 (qn, 2H), 1.39 (s, 2H), 1.27/1.21 (d+d, 4H), 1.18/1.12 (d+d, 4H), 1.03/0.94 (d+d, 2H), 0.84 (s, 6H); ¹³C NMR (100 MHz, DMSO-d₆) δ ppm 166.8, 159.7, 156.3, 153.6, 150.8, 147.8, 140.2, 137.6, 137.3, 136.0, 130.2, 129.1, 127.7, 123.0, 120.4, 115.6, 114.3, 74.0, 66.8, 62.2, 61.5, 59.0, 55.6, 50.0, 46.9, 46.0, 46.0, 43.3, 39.7, 33.5, 30.1, 24.6, 21.0, 15.5, 10.9; HRMS-ESI (m/z): [M+H]⁺calcd for C₄₁H₅₀ClN₆O₅: 741.3531 found: 741.3530.

### Step D: (4-methoxyphenyl)methyl 6-[3-(1,3-benzothiazol-2-ylamino)-4-methyl-6,7-dihydro-5H-pyrido[2,3-c]pyridazin-8-yl]-3-[ ]-[[3-(2-hydroxyethoxy)-5,7-dimethyl-1-adamantyl]methyl]-5-methyl-pyrazol-4-yl]pyridine-2-carboxylate

The mixture of the product from Step C (4.7 g, 6.3 mmol), 1,3-benzothiazol-2-amine (1.9 g, 2 eq), Pd₂dba₃ (580 mg, 0.1 eq), XantPhos (730 mg, 0.2 eq), and DIPEA (3.3 mL, 3 eq) in cyclohexanol (38 mL) was stirred at 130° C. for 2 h. Purification by column chromatography (silica gel, heptane, EtOAc and MeCN as eluents) afforded the desired product (3.83 g, 71%). ¹H NMR (400 MHz, DMSO-d₆): δ ppm 7.95 (d, 1H), 7.81 (brd, 1H), 7.69 (d, 1H), 7.49 (brs, 1H), 7.39 (s, 1H), 7.35 (m, 1H), 7.19 (m, 2H), 7.16 (m, 1H), 6.91 (m, 2H), 5.10 (s, 2H), 4.46 (t, 1H), 3.99 (m, 2H), 3.85 (s, 2H), 3.75 (s, 3H), 3.40 (m, 2H), 3.34 (t, 2H), 2.85 (t, 2H), 2.32 (s, 3H), 2.11 (s, 3H), 1.99 (m, 2H), 1.45-0.9 (m, 12H), 0.84 (s, 6H); HRMS-ESI (m/z): [M+H]⁺calcd for C₄₅sH₅₅N₈O₅S: 855.4016 found: 855.4011.

### Step E: (4-methoxyphenyl)methyl 6-[3-(1,3-benzothiazol-2-ylamino)-4-methyl-6,7-dihydro-5H-pyrido[2,3-c]pyridazin-8-yl]-3-[ ]-[[3,5-dimethyl-7-[2-(p-tolylsulfonyloxy)ethoxy]-1-adamantyl]methyl]-5-methyl-pyrazol-4-yl]pyridine-2-carboxylate

To the product from Step D (3.83 g, 4.48 mmol) and triethylamine (1.87 mL, 3 eq) in DCM (45 mL) was added p-tolylsulfonyl 4-methylbenzenesulfonate (2.19 g, 1.5 eq) and the reaction mixture was stirred for 2 h. Purification by column chromatography (silica gel, heptane and EtOAc as eluents) afforded 2.5 g (55%) of the desired product. ¹H NMR (400 MHz, DMSO-d₆): δ ppm 7.95 (d, 1H), 7.81 (brs, 1H), 7.76 (m, 2H), 7.45 (brs, 1H), 7.45 (m, 2H), 7.40 (s, 1H), 7.35 (m, 1H), 7.18 (m, 2H), 7.17 (m, 1H), 6.97 (d, 1H), 6.90 (m, 2H), 5.10 (s, 2H), 4.05 (m, 2H), 4.00 (m, 2H), 3.82 (s, 2H), 3.74 (s, 3H), 3.47 (m, 2H), 2.85 (m, 2H), 2.40 (s, 3H), 2.32 (s, 3H), 2.10 (s, 3H), 1.98 (m, 2H), 1.87-1.34 (m, 12H), 0.81 (s, 6H); HRMS-ESI (m/z): [M+H]⁺calcd for CSSH₆₁N₈O₇S₂: 1009.4104 found: 1009.4102.

### Preparation G: Methyl 2-(tert-butoxycarbonylamino)-5-[3-(2-fluoro-4-iodo-phenoxy)propyl]thiazole-4-carboxylate

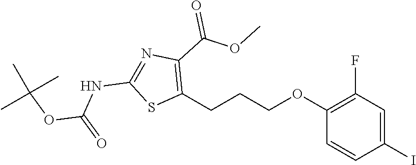 [SMILES: COC(=O)c1nc(NC(=O)OC(C)(C)C)sc1CCCOc1ccc(I)cc1F]

Step A: methyl 2-(tert-butoxycarbonylamino)-5-iodo-thiazole-4-carboxylate

50.00 g methyl 2-(tert-butoxycarbonylamino)thiazole-4-carboxylate (193.55 mmol, 1 equiv) was suspended in 600 mL dry MeCN. 52.25 g N-iodo succinimide (232.30 mmol, ) was added and the resulting mixture was stirred overnight at room temperature.

The reaction mixture was diluted with saturated brine, then it was extracted with EtOAc. The combined organic layers were extracted with 1 M Na₂S2O₃, then with brine again. Then dried over Na₂SO₄, filtered and the filtrate was concentrated under reduced pressure. The crude product was purified via flash chromatography using heptane as eluent to obtain 60 g of the desired product (156 mmol, 80% Yield). ¹H NMR (400 MHz, DMSO-d₆): δ ppm 12.03/11.06 (br s), 3.78 (s, 3H), 1.47 (s, 9H); ¹³C NMR (100 MHz, DMSO-d₆) δ ppm 153.8, 82.5, 77.7, 52.3, 28.3; HRMS-ESI (m/z): [M+H]⁺calcd for C₁₀H₁₄IN₂O₄S: 384.9713; found 384.9708.

### Step B: methyl 2-(tert-butoxycarbonylamino)-5-(3-hydroxyprop-1-ynyl)thiazole-4-carboxylate

A 500 mL oven-dried, one-necked, round-bottom flask was equipped with a PTFE-coated magnetic stirring bar and fitted with a reflux condenser. It was charged with 9.6 g of the product from Step A (25 mmol, 1 equiv), 2.80 g prop-2-yn-1-ol (2.91 mL, 50 mmol, 2 equiv) and 36.10 g DIPA (50 mL, 356.8 mmol, 14.27 equiv) then 125 mL dry THF was added and the system was flushed with argon. After 5 minutes stirring under inert atmosphere 549 mg Pd(PPh₃)₂Cl₂ (1.25 mmol, 0.05 equiv) and 238 mg CuI (1.25 mmol, 0.05 equiv) was added. The resulting mixture was then warmed up to 60° C. and stirred at that temperature until no further conversion was observed. Celite was added to the reaction mixture and the volatiles were removed under reduced pressure. Then it was purified via flash chromatography using heptane and EtOAc as eluents to give 7.30 g of the desired product (23 mmol, 93% Yield) as a yellow solid. ¹H NMR (400 MHz, DMSO-d₆): δ ppm 12.1 (br s, 1H), 5.45 (t, 1H), 4.36 (d, 2H), 3.79 (s, 3H), 1.48 (s, 9H); ¹³C NMR (100 MHz, DMSO-d₆) δ ppm 12.1 (br s, 1H), 5.45 (t, 1H), 4.36 (d, 2H), 3.79 (s, 3H), 1.48 (s, 9H); HRMS-ESI (m/z): [M+H]⁺calcd for C₁₃H₁₇N₂O₅S: 313.0852, found 313.0866.

### Step C: methyl 2-(tert-butoxycarbonylamino)-5-(3-hydroxypropyl)thiazole-4-carboxylate

An 1 L oven-dried pressure bottle equipped with a PTFE-coated magnetic stir bar was charged with 44.75 g of the product from Step B (143.3 mmol, 1 equiv), 7.62 Pd/C (7.17 mmol, 0.05 equiv) in 340 mL ethanol, and then placed under a nitrogen atmosphere using hydrogenation system. After that, it was filled with 4 bar H₂ gas and stirred at rt overnight. Full conversion was observed, but only the olefin product was formed. After filtration of the catalysts through a pad of Celite, the whole procedure was repeated with 5 mol % new catalysts. The resulting mixtures were stirred overnight to get full conversion. Celite was added to the reaction mixtures and the volatiles were removed under reduced pressure. Then it was purified via flash chromatography column using heptane and EtOAc as eluents to give 31.9 g of the desired product (101 mmol, 70.4% Yield) as light-yellow crystals. ¹H NMR (500 MHz, DMSO-d₆): δ ppm 11.61 (br s, 1H), 4.54 (t, 1H), 3.76 (s, 3H), 3.43 (m, 2H), 3.09 (t, 2H), 1.74 (m, 2H), 1.46 (s, 9H); ¹³C NMR (125 MHz, DMSO-d₆) δ ppm 162.8, 143.1, 135.4, 60.3, 51.9, 34.5, 28.3, 23.4; HRMS-ESI (m/z): [M+H]⁺calcd for C13H21N₂05S: 317.1165, found 317.1164 (M+H).

### [731]Step D: methyl 2-(tert-butoxycarbonylamino)-5-[3-(2-fluoro-4-iodo-phenoxy)propyl]thiazole-4-carboxylate

A 250 mL oven-dried, one-necked, round-bottomed flask equipped with a PTFE-coated magnetic stir bar, was charged with 3.40 g 2-fluoro-4-iodo-phenol (14 mmol, 1 equiv), 5.00 g of the product from Step C (16 mmol, 1.1 equiv) and 4.10 g PPh₃ (16 mmol, 1.1 equiv) dissolved in 71 mL dry toluene. After 5 min stirring under nitrogen atmosphere, 3.10 mL DIAD (3.20 g, 16 mmol, 1.1 equiv) was added in one portion while the reaction mixture warmed up. Then the reaction mixture was heated up to 50° C. and stirred at that temperature for 30 min, when the reaction reached complete conversion. The reaction mixture was directly injected onto a preconditioned silica gel column, and then it was purified via flash chromatography using heptane and EtOAc as eluents. The crude product was crystalized from MeOH to give 4.64 g of the desired product (9.24 mmol, 66% Yield). ¹H NMR (500 MHz, DMSO-d₆) δ ppm 11.64 (br s, 1H), 7.59 (dd, 1H), 7.45 (dd, 1H), 6.98 (t, 1H), 4.06 (t, 2H), 3.73 (s, 3H), 3.22 (t, 2H), 2.06 (m, 2H), 1.46 (s, 9H); ¹³C NMR (125 MHz, DMSO-d₆) 6 ppm 134, 124.9, 117.6, 68.2, 51.9, 30.5, 28.3, 23.2; HRMS-ESI (m/z): [M+H]⁺calcd for C19H23N₂05FSI: 537.0350; found 537.0348.

### Preparation H: Methyl 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylate

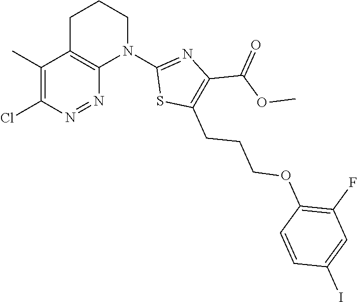 [SMILES: COC(=O)c1nc(N2CCCc3c2nnc(Cl)c3C)sc1CCCOc1ccc(I)cc1F]

### Step A: methyl 2-[[(tert-butoxy)carbonyl][3-(3,6-dichloro-5-methylpyridazin-4-yl)propyl]amino}-5-[3-(2-fluoro-4-iodophenoxy)propyl]-1,3-thiazole-4-carboxylate

Using Mitsunobu General Procedure I starting from 4.85 g Preparation G (9.04 mmol, 1 equiv) as the appropriate carbamate and 2 g Preparation A (9.04 mmol, 1 equiv) as the appropriate alcohol, 4.6 g of the desired product (69% Yield) was obtained. ¹H NMR (500 MHz, DMSO-d₆) δ ppm 7.56 (dd, 1H), 7.44 (dm, 1H), 7.08 (m, 2H), 6.96 (t, 1H), 4.05 (t, 2H), 3.75 (s, 3H), 3.21 (t, 2H), 2.82 (m, 2H), 2.4 (s, 3H), 2.06 (m, 2H), 1.88 (m, 2H), 1.48 (s, 9H); ¹³C NMR (125 MHz, DMSO-d₆) δ ppm 162.7, 157.6, 156.7, 156.5/153.2, 152.2, 147, 142.1, 139.8, 134, 124.9, 117.6, 84, 82.4, 68.1, 52.1, 46.1, 30.4, 28.1, 27.5, 25.8, 23.1, 16.4; HRMS-ESI (m/z): [M+H]⁺calcd for C₂₇H₃₁Cl₂FIN₄O₅S: 739.0415, found 739.0395.

### Step B: methyl 2-[3-(3,6-dichloro-5-methyl-pyridazin-4-yl)propylamino]-5-[3-(2-fluoro-4-iodo-phenoxy)propyl]thiazole-4-carboxylate

Using Deprotection with HFIP General Procedure starting from the product from Step A as the appropriate carbamate, 3.70 g the desired product (97% Yield) was obtained. ¹H NMR (500 MHz, DMSO-d₆) δ ppm 7.71 (t, 1H), 7.59 (dd, 1H), 7.44 (dm, 1H), 6.96 (t, 1H), 4.03 (t, 2H), 3.7 (s, 3H), 3.29 (m, 2H), 3.11 (t, 2H), 2.84 (m, 2H), 2.39 (s, 3H), 2 (m, 2H), 1.76 (m, 2H); ¹³C NMR (125 MHz, DMSO-d₆) δ ppm 164.6, 163, 152.3, 147.1, 134.1, 124.8, 117.6, 82.4, 68.1, 51.9, 44, 30.7, 28, 26.9, 23.3, 16.4; HRMS-ESI (m/z): [M+H]⁺calcd for C₂₂H₂₃Cl₂FIN₄O₃S: 638.9891, found 638.9888.

### Step C: methyl 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylate

A suspension of 3 g of the product from Step B (4.69 mmol, 1 eq) and 1.81 g cesium carbonate (9.3853 mmol, 2 eq.) were stirred at 80° C. for 3 h in 25 mL dry 1,4-dioxane to reach complete conversion. Reaction mixture directly was evaporated to Celite, and then purified by flash chromatography on using DCM-MeOH as eluents to obtain 2.67 g of the title compound (94% Yield). ¹H NMR (500 MHz, DMSO—d) 6 ppm 7.57 (dd, 1H), 7.43 (dm, 1H), 6.97 (t, 1H), 4.23 (t, 2H), 4.08 (t, 2H), 3.77 (s, 3H), 3.22 (t, 2 H), 2.86 (t, 2H), 2.29 (s, 3H), 2.08 (m, 2H), 2.03 (m, 2H); ¹³C NMR (125 MHz, DMSO-d₆) δ ppm 163.1, 155.4, 152.2, 151.6, 151.2, 147, 142.5, 136, 134.8, 134, 128.9, 124.9, 117.6, 82.3, 68.4, 51.9, 46.3, 30.7, 24.2, 23, 19.7, 15.7; HRMS-ESI (m/z): [M+H]⁺calcd for C₂₂H₂₂ClFIN₄O₃S: 603.0124, found 603.0108.

### Preparation I: Methyl 2-[3-(1,3-benzothiazol-2-ylamino)-4-methyl-6,7-dihydro-5H-pyrido[2,3-c]pyridazin-8-yl]-5-[3-[2-fluoro-4-(3-hydroxyprop-1-ynyl)phenoxy]propyl]thiazole-4-carboxylate

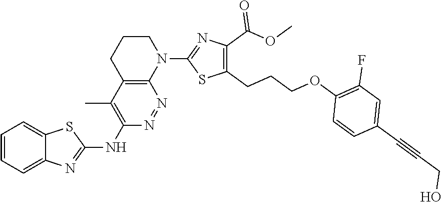 [SMILES: COC(=O)c1nc(N2CCCc3c2nnc(Nc2nc4ccccc4s2)c3C)sc1CCCOc1ccc(C#CCO)cc1F]

### Step A: methyl 5-[3-[4-[3-[tert-butyl(dimethyl)silyl]oxyprop-1-ynyl]-2-fluoro-phenoxy]propyl]-2-(3-chloro-4-methyl-6,7-dihydro-SH-pyrido[2,3-c]pyridazin-8-yl)thiazole-4-carboxylate

Using Sonogashira General Procedure starting from 4.00 g of Preparation H (6.63 mmol, 1.0 eq.) and 2.26 g tert-butyl-dimethyl-prop-2-ynoxy-silane (13.27 mmol, 2 eq.) as the appropriate acetylene, 2.80 g of the desired product (65% Yield) was obtained. ¹H NMR (500 MHz, DMSO-d₆) δ ppm 7.27 (dd, 1H), 7.19 (dd, 1H), 7.14 (t, 1H), 4.51 (s, 1H), 4.25 (m, 2H), 4.12 (t, 2H), 3.77 (s, 3H), 3.24 (t, 2H), 2.87 (t, 2H), 2.3 (s, 3H), 2.1 (quint., 2H), 2.03 (m, 2H), 0.88 (s, 9H), 0.12 (s, 6H); ¹³C NMR (125 MHz, DMSO-d₆) δ ppm 163.0, 128.9, 119.1, 115.5, 68.3, 52.1, 51.9, 46.3, 30.7, 26.2, 24.2, 23.0, 19.7, 15.7, -4.6; HRMS-ESI (m/z): [M+H]⁺calcd for C₃₁H₃₉ClFN₄O₄SSi: 645.2128, found 645.2120.

### Step B: methyl 2-[3-(1,3-benzothiazol-2-ylamino)-4-methyl-6,7-dihydro-SH-pyrido[2,3-c]pyridazin-8-yl]-5-[3-[4-[3-[tert-butyl(dimethyl)silyl]oxyprop-1-ynyl]-2-fluoro-phenoxy]propyl]thiazole-4-carboxylate

Using Buchwald General Procedure II starting from 2.8 g of the product from Step A (4.34 mmol, 1.0 eq.) and 1.30 g 1,3-benzothiazol-2-amine (8.67 mmol, 2.0 eq.), 2.1 g of the desired product (64% Yield) was obtained. ¹H NMR (500 MHz, DMSO-d₆) δ ppm 12.25/10.91 (brs 1H), 7.88 (br, 1H), 7.51 (br, 1H), 7.37 (t, 1H), 7.29 (dd, 1H), 7.2 (t, 1H), 7.2 (dd, 1H), 7.17 (t, 1H), 4.49 (s, 2H), 4.25 (t, 2H), 4.14 (t, 2H), 3.77 (s, 3H), 3.27 (t, 2H), 2.86 (t, 2H), 2.32 (s, 3H), 2.13 (qn, 2H), 2.04 (qn, 2H), 0.87 (s, 9H), 0.1 (s, 6H); ¹³C NMR (125 MHz, DMSO-d₆) δ ppm 163.2, 155.7, 151.6, 148.5, 147.6, 141.5, 128.9, 127.6, 126.5, 122.5, 122.3, 119.1, 116.9, 115.5, 114.8, 88.2, 84, 68.4, 52.1, 51.9, 46.4, 31, 26.2, 24, 23.1, 20.4, 12.9, -4.6; HRMS-ESI (m/z): [M+H]⁺calcd for C₃₈H₄₄FN₆O₄S₂Si: 759.2613, found 759.2609.

### Step C: methyl 2-[3-(1,3-benzothiazol-2-ylamino)-4-methyl-6,7-dihydro-SH-pyrido[2,3-c]pyridazin-8-yl]-5-[3-[2-fluoro-4-(3-hydroxyprop-1-ynyl)phenoxy]propyl]thiazole-4-carboxylate

A 100 mL oven-dried, one-necked, round-bottom flask was equipped with a PTFE-coated magnetic stirring bar and fitted with a reflux condenser. It was charged with 2.10 g of the product from Step B (2.76 mmol, 1.0 eq.) dissolved in 15 mL THF. Then 3.32 mL TBAF (3.32 mmol, 1.2 eq., 1 M in THF) was added dropwise via syringe over a period of 2 minutes, and stirred at that temperature for 30 min. The reaction mixture was quenched with saturated NH₄C₁, then directly evaporated to Celite and it was purified via flash chromatography using heptane- EtOAc as eluents to give 1.6 g of the desired product (90% Yield). ¹H NMR (500 MHz, DMSO-d₆) δ ppm 11.14 (brs, 1H), 7.83 (brd, 1H), 7.49 (brs, 1H), 7.36 (m, 1H), 7.24 (dd, 1H), 7.19 (m, 1H), 7.18 (dm, 1H), 7.15 (t, 1H), 5.08 (t, 1H), 4.28 (m, 2H), 4.27 (d, 2H), 4.17 (t, 2H), 3.8 (s, 3H), 3.29 (m, 2H), 2.89 (m, 2H), 2.35 (s, 3H), 2.15 (m, 2H), 2.07 (m, 2H); HRMS-ESI (m/z): [M+H]⁺calcd for C₃₂H₃₀FN₆O₄S₂: 645.1748, found 645.1738.

### Preparation of PMB protected P5: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-(3-hydroxypropylamino)ethoxy]-5,7-dimethyl-1-adamantyl]methyl]-5-methyl-pyrazol-4-yl]pyridine-2-carboxylate

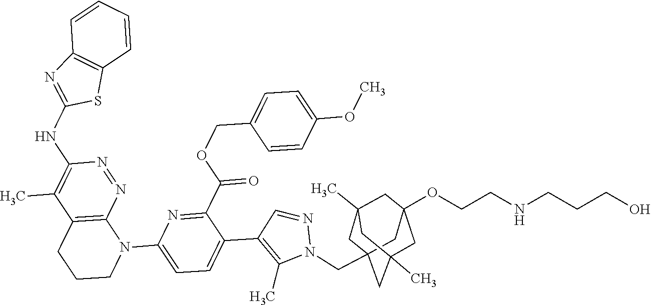 [SMILES: COc1ccc(COC(=O)c2nc(N3CCCc4c3nnc(Nc3nc5ccccc5s3)c4C)ccc2-c2cnn(CC34CC5(C)CC(C)(C3)CC(OCCNCCCO)(C5)C4)c2C)cc1]

To the product from Preparation F in a 1:1 mixture of acetonitrile and N-methyl-2-pyrrolidone (10 ml/mmol) was added 3-aminopropan-1-ol (3-10 eq) and the reaction mixture was stirred at 50° C. for 2-24 h. After the purification of the product by preparative reversed phase chromatography, the desired product was obtained.

HRMS-ESI (m/z): [M+H]⁺calcd for C₅₁H₆₂N₉O₅S: 912.4591, found 912.4581.

### Preparation of P5: 6-[3-(1,3-benzothiazol-2-ylamino)-4-methyl-6,7-dihydro-5H-pyrido[2,3-c]pyridazin-8-yl]-3-[1-[[3-[2-(3-hydroxypropylamino)ethoxy]-5,7-dimethyl-1-adamantyl]methyl]-5-methyl-pyrazol-4-yl]pyridine-2-carboxylic acid

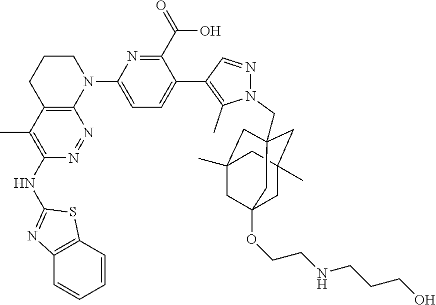 [SMILES: Cc1c(Nc2nc3ccccc3s2)nnc2c1CCCN2c1ccc(-c2cnn(CC34CC5(C)CC(C)(C3)CC(OCCNCCCO)(C5)C4)c2C)c(C(=O)O)n1]

After treating (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-(3-hydroxypropylamino)ethoxy]-5,7-dimethyl-1-adamantyl]methyl]-5-methyl-pyrazol-4-yl]pyridine-2-carboxylate with TFA (15 eq) in DCM (20 mL/mmol) for 18 h, the desired product was obtained by preparative reversed phase chromatography.

HRMS-ESI (m/z): [M+H]⁺calcd for C₄₃H₅₄N₉O₄S: 792.4014, found: 792.4012.

### Preparation of P6: 2-[3-(1,3-Benzothiazol-2-ylamino)-4-methyl-6,7-dihydro-SH-pyrido[2,3-c]pyridazin-8-yl]-5-[3-[4-[3-(dimethylamino)prop-1-ynyl]-2-fluoro-phenoxy]propyl]thiazole-4-carboxylic acid

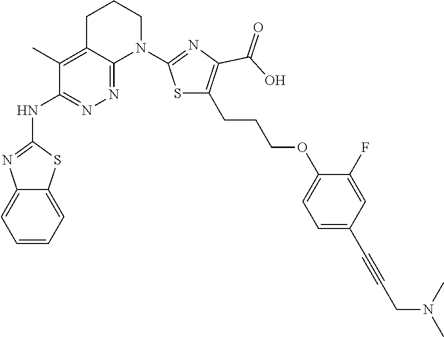 [SMILES: Cc1c(Nc2nc3ccccc3s2)nnc2c1CCCN2c1nc(C(=O)O)c(CCCOc2ccc(C#CCN(C)C)cc2F)s1]

Using Propargylic amine preparation General Procedure starting from Preparation I and dimethylamine as the appropriate amine. Then Hydrolysis General Procedure starting from the appropriate methyl ester, the desired product was obtained. HRMS-ESI (m/z): [M+H]⁺calcd for C₃₄H₃₅FN₇O₃S₂: 672.2221, found 672.2205.

c. Preparation of Bcl2 Payloads

### General Procedures

General procedure 1a-Buchwald

The appropriate aryl bromide (1 eq.), the appropriate aniline (1.1 eq.), 2-di-tert-butylphosphino-2′,4′,6′-triisopropylbiphenyl (0.04 eq.) and NaOBu (2 eq.) were suspended in toluene (3 mL/mmol aryl bromide). The mixture was sparged with N₂ and then Pd₂(dba)₃ (0.04 eq.) was added, and the mixture was heated for 1 h at 110° C. under microwave irradiation or at 70° C. under a N₂ atmosphere until complete conversion was observed. The mixture was allowed to cool to rt, diluted with water and filtered through a celite cartridge. The filtrate was partitioned between EtOAc and water. The phases were separated, and the aqueous phase was extracted with EtOAc. The combined organic phases were dried over MgSO₄ and concentrated in vacuo. The crude material was purified by automated flash chromatography using heptane and EtOAc as eluents.

General procedure 1b - Buchwald

To a solution of the appropriate aryl bromide (1 eq.) and the appropriate aniline (1 eq.) in THF (4.5 mL/mmol aryl bromide) was added NaO′Bu (1 eq.) and chloro(2-di-t-butylphosphino-2′,4′,6′-tri-i-propyl-1,1′-biphenyl)[2-(2-aminoethyl)phenyl]palladium(II) [t-BuXPhos Palladacycle Gen. 1](0.04 eq.) and the mixture was stirred at rt under N₂until complete conversion was observed. The mixture was diluted with water and the organic layer was extracted with EtOAc. The combined organic layers were washed with brine, dried over MgSO₄ and concentrated in vacuo. The crude material was purified by automated flash chromatography using heptane or MeOH and EtOAc as eluents.

General procedure 2a - Amide coupling

To a solution of the appropriate acid (1 eq.) in DCE (12 mL/mmol acid) was added 4A molecular sieves followed by 1-chloro-N,N,2-trimethyl-1-propenylamine (2 eq.) and then the mixture was stirred under N₂ at rt for 30 mins. A solution of the appropriate aniline (1.5 eq.) and pyridine (2 eq.) in DCE (2.5 mL/mmol aniline) was added and the mixture stirred at 80° C. under N₂ for 18 h. The mixture was allowed to cool to rt and partitioned between DCM and water. The phases were separated, and the organic phase was washed with sat. aq. NaHCO₃ solution, dried over MgSO₄ and concentrated in vacuo. The crude material was purified by automated flash chromatography using heptane and EtOAc or DCM and MeOH as eluents.

General procedure 2b - Amide coupling

To a solution of the appropriate acid (1 eq.) in DCM (12 mL/mmol acid) was added oxalyl dichloride (1.7 eq.) and then the mixture was stirred under N₂ at rt for 30 mins. The solution was concentrated in vacuo at rt then the appropriate aniline (1.2 eq.) in DCE (12 mL/mmol acid) was added and the mixture was stirred at 80° C. under N₂ for 4-18 h. The reaction mixture was concentrated in vacuo and the crude material was purified by automated flash chromatography using DCM and MeOH as eluents.

General procedure 4a - Amide coupling

To a solution of the appropriate amine (1 eq.) in DMF (6 mL/mmol amine) was added the appropriate carboxylic acid (1.5 eq.) followed by DIPEA (2 eq.) and HATU (1 eq.) and the mixture was stirred at rt until complete conversion was observed. The reaction mixture was partitioned between DCM and water. The phases were separated and the organic phase dried over MgSO₄ and concentrated. Purification by automated flash chromatography using DCM and MeOH as eluents was followed by preparative HPLC automated flash chromatography at pH 9 or pH 4, using water and MeCN as eluents.

General procedure 7a —O-Alkylation

A stirred suspension of the appropriate phenol (1 eq.) and K₂CO₃ (5 eq.) in MeCN (5-10 mL/mmol) was heated at 70° C. for 20 min. The appropriate alkyl halide (1.6 eq.) was added, and heating continued until complete conversion was observed. The mixture was allowed to cool to rt and filtered, washing with EtOAc. The filtrate was concentrated in vacuo and the residue was purified by automated flash chromatography using heptane and EtOAc or DCM and MeOH as eluents.

General procedure 8 - Ester hydrolysis

A solution of the appropriate ester (1 eq.) in MeOH (3-11 mL/mmol) was treated with 2 M aq. NaOH solution (2 eq.) and stirred at rt for 24 h. The MeOH was removed in vacuo and the aqueous residue was neutralised with 2 M aq. HCl solution and then purified by reverse phase automated flash chromatography using water and MeCN as eluents.

### Preparation I: 2-benzyloxycarbonyl-6-(4-ethoxycarbonyl-1,5-dimethyl-pyrrol-2-yl)-3,4-dihydro-1H-isoquinoline-7-carboxylic acid

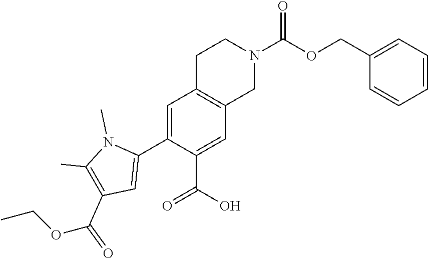 [SMILES: CCOC(=O)c1cc(-c2cc3c(cc2C(=O)O)CN(C(=O)OCc2ccccc2)CC3)n(C)c1C]

The synthesis of the title compound is described in WO2015/011164 A1, in Example 805 Step B.

### Preparation IIa: 5-[2-(tert-butoxycarbonyl)-7-{[(3R)-3-methyl-3,4-dihydro-1H-isoquinolin-2-yl]carbonyl}-3,4-dihydro-1H-isoquinolin-6-yl]-1,2-dimethylpyrrole-3-carboxylic acid

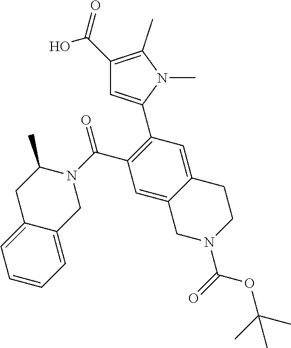 [SMILES: Cc1c(C(=O)O)cc(-c2cc3c(cc2C(=O)N2Cc4ccccc4C[C@H]2C)CN(C(=O)OC(C)(C)C)CC3)n1C]

### Step A: (3R)-3-methyl-1,2,3,4-tetrahydroisoquinoline hydrochloride

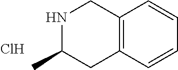

The synthesis of the title compound is described in WO2015/011164 A1 at Preparation 2b.

### Step B: benzyl 6-[4-(ethoxycarbonyl)-1,5-dimethylpyrrol-2-yl]-7-[[(3R)-3-methyl-3,4-dihydro-]H-isoquinolin-2-yl]carbonyl}-3,4-dihydro-]H-isoquinoline-2-carboxylate

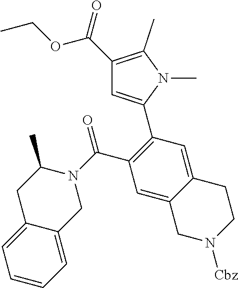 [SMILES: CCOC(=O)c1cc(-c2cc3c(cc2C(=O)N2Cc4ccccc4C[C@H]2C)CN(C(=O)OCc2ccccc2)CC3)n(C)c1C]

To a solution of Preparation I (3 g, 6.3 mmol, 1 eq.) and the product from Step A (1.27 g, 6.93 mmol, 1.1 eq.) in DMF (30 mL) was added DIPEA (3.13 mL, 18.89 mmol, 3 eq.) and PyBop (3.6 g, 6.93 mmol, 1.1 eq.) and the mixture stirred at rt for 12 h. The mixture was diluted with water (120 mL), stirred for 15 mins and the resultant cream precipitate was collected by filtration, washing with water. The filter cake was dissolved in DCM and washed with sat. aq. NaHCO₃ solution and water, dried over MgSO₄ and concentrated in vacuo to afford the title product (3.7 g, 6.11 mmol, 97%).

HRMS calcd for C37H₃₉N₃O₅: 605.289, found 606.296 [M+H]⁺

¹H NMR (400 MHz, DMSO-d6) 6 ppm: 7.52-6.75 (m, 11H), 6.43-6.00 (m, 1H), 5.39-1.95 (m, 21H), 1.31-0.49 (m, 6H).

### Step C: ethyl 1,2-dimethyl-5-(7-[[(3R)-3-methyl-3,4-dihydro-]H-isoquinolin-2-yl]carbonyl}-1,2,3,4-tetrahydroisoquinolin-6-yl)pyrrole-3-carboxylate

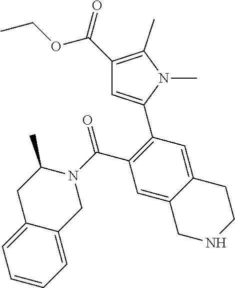 [SMILES: CCOC(=O)c1cc(-c2cc3c(cc2C(=O)N2Cc4ccccc4C[C@H]2C)CNCC3)n(C)c1C]

To a solution of the product from Step B (3.7 g, 6.11 mmol, 1 eq.) in MeOH (70 mL) and EtOH (20 mL) was added 10% Pd/C (100 mg). The mixture was evacuated and backfilled with N₂, then evacuated and flushed with H2 and then shaken at rt for 6 h under an atmosphere of H2. The mixture was filtered through a celite cartridge, washing with EtOH. The solvent was removed in vacuo to afford the title product (2.94 g, quant.).

HRMS calcd for C₂₉H₃₃N₃O₃: 471.252, found 472.259 [M+H]⁺

¹H NMR (400 MHz, DMSO-d6) 6 ppm: 7.24-6.81 (m, 6H), 6.40-5.99 (m, 1H), 5.34-1.93 (m, 19H), 1.30-0.52 (m, 7H).

### Step D: tert-butyl 6-[4-(ethoxycarbonyl)-1,5-dimethylpyrrol-2-yl]-7-[[(3R)-3-methyl-3,4-dihydro-]H-isoquinolin-2-yl]carbonyl}-3,4-dihydro-]H-isoquinoline-2-carboxylate

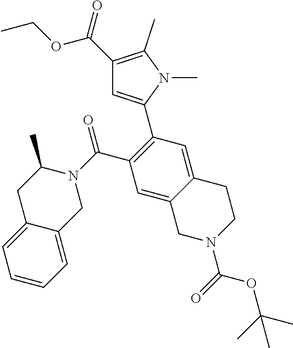 [SMILES: CCOC(=O)c1cc(-c2cc3c(cc2C(=O)N2Cc4ccccc4C[C@H]2C)CN(C(=O)OC(C)(C)C)CC3)n(C)c1C]

To a solution of the product from Step C (2.94 g, 6.23 mmol, 1 eq.) in THF (45 mL) and water (6 mL) was added bis(tert-butyl) dicarbonate (1.43 g, 6.55 mmol, 1.05 eq.) followed by TEA (1.73 mL, 12.47 mmol, 2 eq.) and the mixture stirred at rt for 12 h. The solvents were removed in vacuo and the residue partitioned between EtOAc and sat aq. NH₄Cl solution. The phases were separated, and the organic phase was washed with sat. aq. NaCl solution, dried over MgSO₄ and concentrated in vacuo to afford the title product (3.39 g, 5.93 mmol, 95%).

HRMS calcd for C₃₄H₄₁N₃O₅: 571.305, found 572.315 [M+H]⁺

¹H NMR (400 MHz, DMSO-d6) 6 ppm: 7.33-6.79 (m, 6H), 6.41-6.00 (m, 1H), 5.34-1.99 (m, 19H), 1.51-1.37 (m, 9H), 1.28-0.52 (m, 6H).

### Step E: 5-[2-(tert-butoxycarbonyl)-7-[[(3R)-3-methyl-3,4-dihydro-]H-isoquinolin-2-yl]carbonyl}-3,4-dihydro-]H-isoquinolin-6-yl]-1,2-dimethylpyrrole-3-carboxylic acid

LiOH·H₂O (995 mg, 23.72 mmol, 4 eq.) was added to a solution of the product from Step D (3.39 g, 5.93 mmol, 1 eq.) in a mixture of MeOH (40 mL) and water (20 mL) and heated at 100° C. for 24 h. The mixture was allowed to cool to rt and MeOH removed in vacuo. The aqueous residue was acidified with 1 M aq. HCl solution to pH 5, diluted with water (10 mL) and extracted with DCM. The combined organic extracts were dried over MgSO₄ and concentrated in vacuo. Purification by automated flash chromatography eluting with a gradient of 0-6% MeOH in DCM afforded the title product (3.1 g, 5.7 mmol, 96%).

HRMS calcd for C₃₂H₃₇N₃O₅: 543.273, found 544.283 [M+H]⁺

¹H NMR (400 MHz, DMSO-d6) 6 ppm: 11.53 (s, 1H), 7.32-6.80 (m, 6H), 6.38-6.06 (m, 1H), 5.37-1.89 (m, 17H), 1.50-1.38 (m, 9H), 1.08-0.50 (m, 3H).

### Preparation IIIa: 4-[(tert-butyldimethylsilyl)oxy]aniline

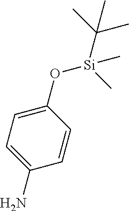

To a solution of imidazole (13.72 g, 201.6 mmol, 2.5 eq.) and 4-aminophenol (8.8 g, 80.64 mmol, 1 eq.) in MeCN (150 mL), cooled to 0° C. under N₂, was added TBDMSCl (12.76 g, 84.67 mmol, 1.05 eq.). The mixture was allowed to warm to rt and stirred for 4 h. The mixture was partitioned between EtOAc and water. The phases were separated, and the organic phase was washed with brine, dried over MgSO₄ and concentrated in vacuo. The residue was diluted with heptane (40 mL) and left to stir for 1 h. The precipitate was removed by filtration and the filtrate concentrated in vacuo to afford the title product (19.6 g, quant.).

HRMS calcd for (C12H21NOSi): 223.139, found 224.162 [M+H]⁺

¹H NMR (400 MHz, DMSO-d6) 6 ppm: 6.56-6.48 (m, 2H), 6.48-6.40 (m, 2H), 4.87 (br s, 2H), 0.92 (s, 9H), 0.10 (s, 6H).

### Preparation IVa: N-(5-cyano-1,2-dimethylpyrrol-3-yl)-N-(4-hydroxyphenyl)-1,2-dimethyl-5-(7-{[(3R)-3-methyl-3,4-dihydro-1H-isoquinolin-2-yl]carbonyl}-1,2,3,4-tetrahydroisoquinolin-6-yl)pyrrole-3-carboxamide hydrochloride

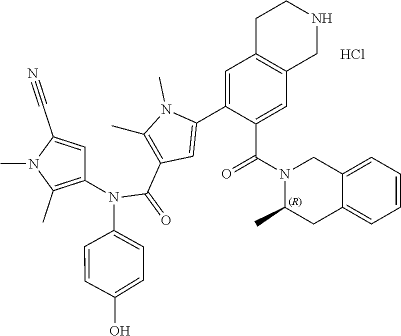

### Step A: 4-bromo-1,5-dimethylpyrrole-2-carbonitrile

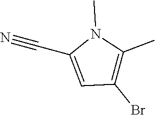

A solution of Br₂ (6.74 mL, 131.08 mmol, 1.05 eq.) in AcOH (70 mL) was added dropwise to a solution of 1,5-dimethylpyrrole-2-carbonitrile (15 g, 124.84 mmol, 1 eq.) in AcOH (250 mL), cooled to 10° C. The mixture was stirred at 10° C. for 1 h, and then allowed to warm to rt, and again stirred for 3 h. The mixture was poured into ice water (500 mL) and the resulting precipitate was collected by filtration, washed with water, and dried in vacuo to afford the title product, (23.7 g, 119.07 mmol, 95%).

¹H NMR (400 MHz, DMSO-d6) 6 ppm: 7.05 (s, 1H), 3.65 (s, 3H), 2.22 (s, 3H).

### Step B: 4-([4-[(tert-butyldimethylsilyl)oxy]phenyl}amino)-1,5-dimethylpyrrole-2-carbonitrile

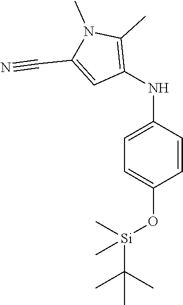 [SMILES: Cc1c(Nc2ccc(O[Si](C)(C)C(C)(C)C)cc2)cc(C#N)n1C]

Using General procedure 1a and the product from Step A (6.1 g, 30.65 mmol, 1 eq.) as the appropriate aryl bromide and Preparation IIIa (7.19 g, 32.18 mmol, 1.05 eq.) as the appropriate aniline afforded the title product (8.2 g, 24 mmol, 78%).

HRMS calcd for C₁₉H₂₇N₃OSi: 341.192, found 342.207 [M+H]⁺

¹H NMR (400 MHz, DMSO-d6) 6 ppm: 6.87 (s, 1H), 6.73 (s, 1H), 6.64-6.56 (m, 2H), 6.54-6.46 (m, 2H), 3.61 (s, 3H), 2.09 (s, 3H), 0.92 (s, 9H), 0.12 (s, 6H).

### Step C: tert-butyl 6-[4-([4-[(tert-butyldimethylsilyl)oxy]phenyl}(5-cyano-1,2-dimethylpyrrol-3-yl)carbamoyl)-1,5-dimethylpyrrol-2-yl]-7-[[(3R)-3-methyl-3, 4-dihydro-]H-isoquinolin-2-yl]carbonyl}-3,4-dihydro-]H-isoquinoline-2-carboxylate

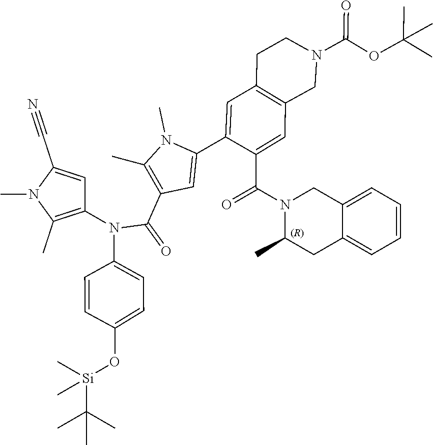

The title compound was prepared according to General procedure 2a using Preparation IIa (2.5 g, 4.6 mmol, 1 eq.) as the appropriate acid and the product from Step B (3.14 g, 9.2 mmol, 2 eq.) as the appropriate aniline. Purification by automated flash chromatography eluting with a gradient of 0-6% MeOH in DCM afforded the title product (2.49 g, 2.87 mmol, 62%).

HRMS calcd for C₅₁H₆₂N₆O₅Si: 866.455, found 867.465 [M+H]⁺

### Step D: N-(5-cyano-1,2-dimethylpyrrol-3-yl)-N-(4-hydroxyphenyl)-1,2-dimethyl-5-(7-[[(3R)-3-methyl-3,4-dihydro-]H-isoquinolin-2-yl]carbonyl}-1,2,3,4-tetrahydroisoquinolin-6-yl)pyrrole-3-carboxamide hydrochloride

A solution of the product from Step C (2.8 g, 3.23 mmol, 1 eq.) in MeOH (5 mL) was treated with 3 M HCl in MeOH (10 mL, 30 mmol) and stirred at rt for 3 h. The solvents were removed in vacuo and then dried under high vacuum to afford the title product (2.53 g, quant.).

HRMS calcd for C₄₀H₄₀N₆O₃: 652.316, found 653.326 [M+H]⁺

### Preparation Va: {4-[2-(morpholin-4-yl)ethoxy]phenyl}acetic acid

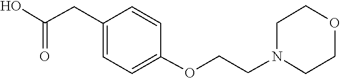 [SMILES: O=C(O)Cc1ccc(OCCN2CCOCC2)cc1]

Step A: methyl 2-[4-[2-(morpholin-4-yl)ethoxy]phenyl}acetate

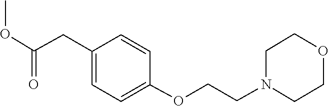 [SMILES: COC(=O)Cc1ccc(OCCN2CCOCC2)cc1]

Using General procedure 7a and methyl 4-hydroxyphenylacetate (2.5 g, 15.04 mmol, 1 eq.) as the appropriate phenol and 4-(2-chloroethyl)morpholine hydrochloride (4.48 g, 24.07 mmol, 1.6 eq.) as the appropriate alkyl halide afforded the title product (3.24 g, 11.59 mmol, 77%).

LRMS calcd for (C₁₅H₂₁NO₄): 279.2, found 280.2 [M+H]⁺

¹H NMR (400 MHz, DMSO-d6) 6 ppm: 7.20-7.12 (m, 2H), 6.93-6.85 (m, 2H), 4.06 (t, J=5.8 Hz, 2H), 3.60 (s, 3H), 3.59 (s, 2H), 3.59-3.56 (m, 4H), 2.68 (t, J=5.8 Hz, 2H), 2.50-2.43 (m, 4H).

Step B: [4-[2-(morpholin-4-yl)ethoxy]phenyl}acetic acid

The product from Step A (3.26 g, 11.67 mmol, 1 eq.) was hydrolysed using General procedure 8 to afford the title product (3.1 g, 11.63 mmol, quant).

LRMS calcd for (C₁₄H₁₉NO₄): 265.1, found 266.2 [M+H]⁺

¹H NMR (400 MHz, DMSO-d6) 6 ppm: 7.15-7.09 (m, 2H), 6.87-6.79 (m, 2H), 4.04 (t, J=5.8 Hz, 2H), 3.60-3.54 (m, 4H), 3.34 (s, 2H), 2.66 (t, J=5.8 Hz, 2H), 2.49-2.42 (m, 4H).

### Preparation VIa: 1,2-dimethyl-5-(7-{[(3R)-3-methyl-3,4-dihydro-1H-isoquinolin-2-yl]carbonyl}-2-(2-{4-[2-(morpholin-4-yl)ethoxy]phenyl}acetyl)-3,4-dihydro-1H-isoquinolin-6-yl)pyrrole-3-carboxylic acid

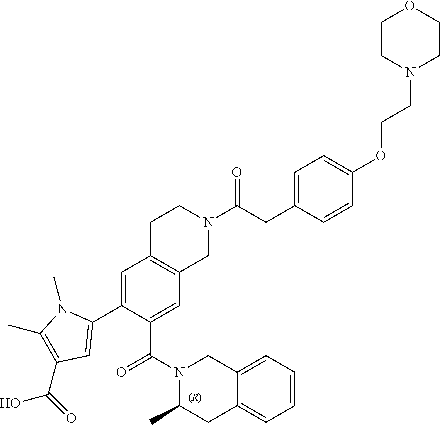

### Step A: 1,2-dimethyl-5-(7-[[(3R)-3-methyl-3,4-dihydro-]H-isoquinolin-2-yl]carbonyl}-],2,3,4-tetrahydroisoquinolin-6-yl)pyrrole-3-carboxylic acid hydrochloride

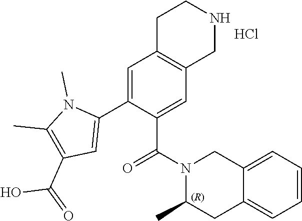

A solution of Preparation IIa (3 g, 5.52 mmol, 1 eq.) in MeOH (20 mL) was treated with 3 M HCl in MeOH (20 mL, 60 mmol) and stirred at rt for 3 h. The solvents were removed in vacuo and dried under vacuum to afford the title product (2.71 g, quant.).

LRMS calcd for C₂₇H₂₉N₃O₃: 443.2, found 444.4 [M+H]⁺

¹H NMR (400 MHz, DMSO-d6) 6 ppm: 9.79-9.40 (br m, 2H), 7.42-6.79 (m, 6H), 6.43-6.04 (m, 1H), 5.37-1.87 (m, 17H), 1.14-0.46 (m, 3H).

Step B: 1,2-dimethyl-5-(7-[[(3R)-3-methyl-3,4-dihydro-]H-isoquinolin-2-yl]carbonyl}-2-(2-[4-[2-(morpholin-4-yl)ethoxy]phenyl}acetyl)-3,4-dihydro-]H-isoquinolin-6-yl)pyrrole-3-carboxylic acid

To a solution of Preparation Va (1 g, 3.77 mmol, 1 eq) in anhydrous DCM (10 mL) under N₂, was slowly added 2 M oxalyl chloride solution in DCM (0.43 mL, 4.52 mmol, 1.2 eq.) followed by DMF (1 drop), and the mixture stirred for 1 h. The solvents were removed in vacuo, the acid chloride intermediate was dissolved in anhydrous DCM (7 mL) and added dropwise to a stirred solution of the product from Step A (1.63 g, 3.39 mmol, 0.9 eq.) and DIPEA (2.5 mL, 15.08 mmol, 4 eq.) in anhydrous DCM (10 mL) under N₂. The mixture was stirred at rt for 2 h, then quenched with MeOH (5 mL) and concentrated in vacuo. Purification by automated flash chromatography eluting with a gradient of 0-8% MeOH in DCM afforded the title product (1.2 g, 1.74 mmol, 46%).

LRMS calcd for (C₄₁H₄₆N₄O₆): 690.3, found 691.6 [M+H]⁺

¹H NMR (400 MHz, DMSO-d6) 6 ppm: 11.53 (s, 1H), 7.32-6.78 (m, 10H), 6.39-6.05 (m, 1H), 5.35-1.88 (m, 31H), 1.11-0.48 (m, 3H).

### [828]Preparation VIIb: N-[4-[tert-butyl(dimethyl)silyl]oxyphenyl]-N-(5-cyano-1,2-dimethyl-pyrrol-3-yl)-1,2-dimethyl-5-[7-[(3R)-3-methyl-3,4-dihydro-1H-isoquinoline-2-carbonyl]-1,2,3,4-tetrahydroisoquinolin-6-yl]pyrrole-3-carboxamide

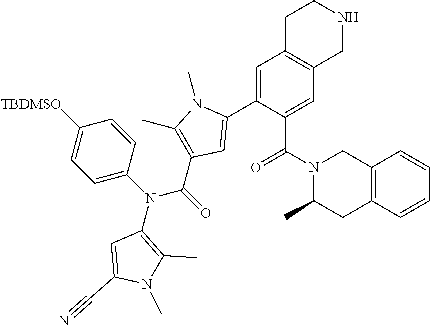 [SMILES: Cc1c(N(C(=O)c2cc(-c3cc4c(cc3C(=O)N3Cc5ccccc5C[C@H]3C)CNCC4)n(C)c2C)c2ccc(O[Si](C)(C)C(C)(C)C)cc2)cc(C#N)n1C]

### Step A: 1,2-dimethyl-5-[7-[(3R)-3-methyl-3,4-dihydro-]H-isoquinoline-2-carbonyl]-1,2,3,4-tetrahydroisoquinolin-6-yl]pyrrole-3-carboxylic acid

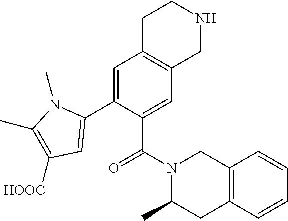 [SMILES: Cc1c(C(=O)O)cc(-c2cc3c(cc2C(=O)N2Cc4ccccc4C[C@H]2C)CNCC3)n1C]

To a solution of Preparation I (45.9 g, 96.3 mmol, 1 eq.) in DMF (240 mL) was added TBTU (34.1 g, 106 mmol, 1.1 eq.) followed by TEA (40.5 mL, 291 mmol, 3 eq.). After stirring for 10 mins the product from Step A of Preparation IIa (18.6 g, 101 mmol, 1.05 eq.) was added and the mixture was stirred at rt for 1 h then it was poured into water (1500 mL) and the precipitates were filtered out, washed with water. This crude intermediate was dissolved in methanol (250 mL) and water (25 mL) then NaOH (28 g, 700 mmol, 7.3 eq.) was added and mixture was stirred at reflux temperature for 18 h. Evaporated at reduced pressure, then the pH was adjusted to 6 by the addition of cc. aq. HCl. The product was extracted with DCM/IPA=3/1 (3×400 mL). The organic phase was dried over MgSO₄ and concentrated in vacuo. The residue was triturated in diethyl ether/acetonitrile. The formed yellow powder was filtered out then dried in vacuo to afford the title product (38.51 g, 86.8 mmol, 90%).

¹H NMR (500 MHz, DMSO-d6) δ ppm 7.35-6.8 (m, 6H), 6.35/6.28/6.13 (s/s/s, 1H), 5.34-3.23 (m, 6H), 5.01/4.88/3.78 (m/m/m, 1H), 3.46/3.43/3.38/3.15 (s/s/s/s, 3H), 3.12-2.98 (m, 2H), 3.02-2.05 (m, 2H), 2.49/2.41/1.96 (s/s/s, 3H), 1.03/0.85/0.74/0.56 (d/d/d/d, 3H); ¹³C NMR (125 MHz, DMSO-d6) δ ppm 168.9/168.4/168.2, 166.5/166.3/166.2, 111.3/111.2/110.8, 48.3/48.1/43/42.2, 34.4/34.2/34/32.6, 32.2/32.1/31.9, 25.4, 18.6/16.8/16.4, 11.8/11.7/11.2.

HRMS-ESI (m/z) [M+H]⁺calcd for C₂₇H₃₀N₃O₃: 444.2282, found 444.2267

### Step B: 5-[2-(9H-fluoren-9-ylmethoxycarbonyl)-7-[(3R)-3-methyl-3,4-dihydro-]H-isoquinoline-2-carbonyl]-3,4-dihydro-]H-isoquinolin-6-yl]-1,2-dimethyl-pyrrole-3-carboxylic acid

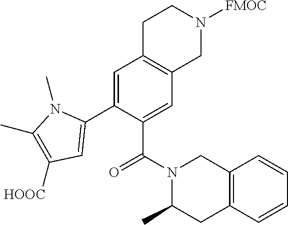

To the biphasic mixture of the product from Step A (13.96 g, 28.33 mmol, 1 eq.) dissolved in dioxane (160 mL) and NaHCO₃ (5.47 g, 65.2 mmol, 2.3 eq.) dissolved in water (160 mL) 9H-fluoren-9-ylmethyl carbonochloridate (8.06 g, 31.2 mmol, 1.1 eq.) was added dropwise and the mixture was stirred at rt for 24 h. To the reaction mixture 2M aq. HCl (42.5 mL; 85 mmol, 3 eq.) was added dropwise then after stirring for 10 min it was extracted with DCM. The phases were separated, and the organic phase was washed with sat. aq. NaCl solution, dried over MgSO₄ and concentrated in vacuo. The crude material was purified by automated flash chromatography using DCM and EtOAc as eluents affording the title product (14.77 g, 22.2 mmol, 78%).

¹H NMR (500 MHz, DMSO-d6) δ ppm 11.18 (br., 1H), 7.95-7.26 (br., 8H), 7.26-6.8 (br., 6H), 6.52-6.06 (br., 1H), 5.09-3.74 (br., 3H), 4.54 (br., 2H), 4.47 (d, 2H), 4.31 (t, 1H), 3.59 (br., 2H), 3.5-3.19 (br., 3H), 2.8 (t, 2H), 2.55-1.93 (br., 3H), 2.45 (br., 2H), 1.2-0.41 (br., 3H); ¹³C NMR (125 MHz, DMSO-d6) δ ppm 111.1, 67.2, 47.5, 45.7, 41.6, 34.6, 32, 28.3, 16.8, 11.6.

HRMS-ESI (m/z) [M+H]⁺calcd for C₄₂H₄₀N₃O₅: 666.2962, found 666.2961

### Step C: 9H-fluoren-9-ylmethyl 6-[4-[[4-[tert-butyl(dimethyl)silyl]oxyphenyl]-(5-cyano-1,2-dimethyl-pyrrol-3-yl)carbamoyl]-1,5-dimethyl-pyrrol-2-yl]-7-[(3R)-3-methyl-3,4-dihydro-]H-isoquinoline-2-carbonyl]-3,4-dihydro-]H-isoquinoline-2-carboxylate

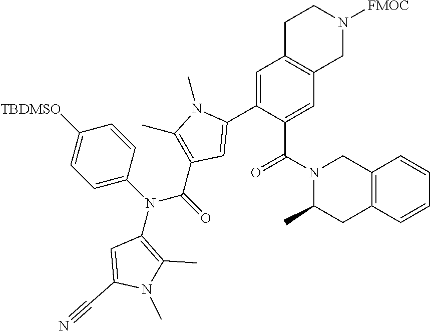

Using General procedure 2b and the product from Step B (10.67 g, 16.03 mmol, 1 eq.) as the appropriate acid, oxalyl dichloride (2.17 mL, 25.6 mmol, 1.6 eq.) and the product from Step B of Preparation IVa (6.57 g, 19.2 mmol, 1.2 eq.) as the appropriate aniline afforded the title product (8.12 g, 8.21 mmol, 51%).

¹H NMR (500 MHz, DMSO-d6) δ ppm 8.05-6.43 (m, 19H), 5.7-4.95 (s, 1H), 5.12-2.26 (m, 14 H), 3.71-3 (s, 6H), 2.48-1.75 (s, 6H), 1.13-0.44 (d, 3H), 0.92-0.77 (s, 9H), 0.15-0.05 (s, 6H).

HRMS-ESI (m/z) [M+H]⁺calcd for C₆₁H₆₅N₆O₅Si: 989.4780, found 989.4779

Step D: N-[4-[tert-butyl(dimethyl)silyl]oxyphenyl]-N-(5-cyano-1,2-dimethyl-pyrrol-3-yl)-1,2-dimethyl-5-[7-[(3R)-3-methyl-3,4-dihydro-]H-isoquinoline-2-carbonyl]-1,2,3,4-tetrahydroisoquinolin-6-yl]pyrrole-3-carboxamide

To a solution of the product from Step C (8.12 g, 8.21 mmol, 1 eq.) in DCM (41 mL) morpholine (41 mL, 475 mmol, 58 eq.) was added then the mixture was stirred at rt for 18 h. The reaction mixture was concentrated in vacuo and the residue was partitioned between DCM and water. The phases were separated, and the organic phase was washed with sat. aq. NaCl solution, dried over MgSO₄ and concentrated in vacuo. The crude material was purified by automated flash chromatography using DCM and MeOH as eluents to afford the title product (4.51 g, 5.88 mmol, 72%).

¹H NMR (500 MHz, DMSO-d6) δ ppm 7.36-6.35 (m, 11H), 5.55-4.91 (s, 1H), 5.37-1.97 (m, 12 H), 3.72-2.96 (s, 6H), 2.48-1.75 (s, 6H), 1.16-0.39 (d, 3H), 1-0.76 (s, 9H), 0.17-0 (s, 6H).

HRMS-ESI (m/z) [M+H]⁺calcd for C₄₆H₅₅N₆O₃Si: 767.4099, found 767.4103

### Preparation of P2: 5-[5-chloro-2-[(3R)-3-[3-(dimethylamino)propyl]-3,4-dihydro-1H-isoquinoline-2-carbonyl]phenyl]-N-(5-cyano-1,2-dimethyl-pyrrol-3-yl)-N-(4-hydroxyphenyl)-1,2-dimethyl-pyrrole-3-carboxamide

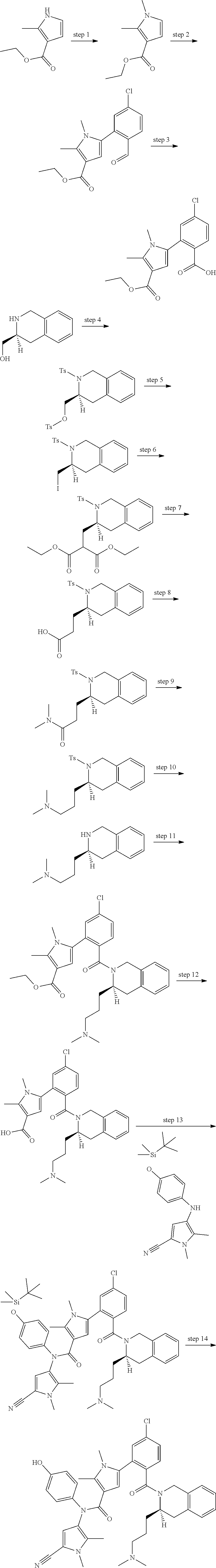

### 46]Step 1: Ethyl 1,2-dimethyl-]H-pyrrole-3-carboxylate

To a solution of ethyl 2-methyl-1H-pyrrole-3-carboxylate (10 g, 65.3 mmol) and methyl iodide (8.95 mL, 130.6 mmol) in 70 mL of dimethylformamide cooled at 0° C. is added, in three portions, sodium hydride 60% w/w (2.6 g, 65.3 mmol). The reaction mixture is stirred at 0° C. for 1 hour. Then, the reaction mixture is hydrolysed by addition of 420 mL of ice-cold water, diluted with ethyl acetate, and successively washed with 0.1M aqueous hydrochloric acid (HCl) solution, saturated aqueous LiCl solution and then brine. The organic phase is dried over MgSO₄, filtered, concentrated to dryness and purified by chromatography over silica gel (petroleum ether/AcOEt gradient).

¹H NMR: 8 (400 MHz; dmso-d6; 300K): 6.65 (d, 1H); 6.3 (id, 1H); 4.1 (1q, 2H); 3.5 (s, 3H); 2.4 (s, 3H); 1.5 (it, 3H).

Step 2: Ethyl 5-(5-chloro-2-formylphenyl)-1,2-dimethyl-]H-pyrrole-3-carboxylate

To a solution of the compound obtained in Step 1 (10.5 g, 62.8 mmol) in 65 mL of N,N-dimethylacetamide there are successively added 2-bromo-4-chlorobenzaldehyde (15.2 g, 69 mmol), potassium acetate (12.3 g, 125.6 mmol) and then the reaction mixture is stirred under argon for 20 minutes. There is added PdCl₂(PPh₃)₂(2.2 g, 3.14 mmol). The reaction mixture is heated at 130° C. overnight, allowed to cool down to room temperature and it is diluted with dichloromethane. Animal charcoal is added (20g), the suspension is stirred at room temperature for 1 hour and filtered. The organic phase is washed with water, dried over MgSO₄ and concentrated to dryness. The crude product thereby obtained is purified by chromatography over silica gel (petroleum ether/AcOEt gradient). The title product is obtained in the form of a solid.

¹H NMR: 8 (400 MHz; dmso-d6; 300K): 9.8 (s, 1H); 7.91-7.69-7.61 (d, 3H); 6.5 (s, 1H); 4.2 (q, 2H); 3.4 (s, 3H); 2.55 (s, 3H); 1.28 (t, 3H).

Step 3: 4-Chloro-2-[4-(ethoxycarbonyl)-1,5-dimethyl-]H-pyrrol-2-yl]benzoic acid

A solution is prepared containing the compound obtained in Step 2 (12.8 g, 42 mmol) and 2-methyl-2-butene (35.7 mL, 336 mmol) in a mixture containing 20 mL of acetone and 20 mL of tetrahydrofuran. There are added, dropwise, 200 mL of an aqueous solution containing a mixture of sodium chlorite (NaClO₂, 13.3 g, 147 mmol) and sodium hydrogen phosphate (NaHPO₄, 14.5 g, 105 mmol). The reaction mixture is vigorously stirred at room temperature for 7 hours and concentrated to remove the acetone. Ethyl acetate is added, the organic phase is washed with water, dried over MgSO₄ and then concentrated to dryness. The residue is then taken up in a minimum of ethyl ether. The solid then obtained is filtered off, washed with ether and then dried in vacuo at 40° C. overnight. The title product is obtained in the form of a solid, which is subsequently used without being otherwise purified.

¹H NMR: 8 (400 MHz; dmso-d6; 300K): 13 (m, 1H); 7.85-7.41 (m, 3H); 6.3 (s, 1H); 4.15 (q, 2H); 3.25 (s, 3H); 2.5 (s, 3H); 1.25 (t, 3H).

Step 4: [(3S)-2-[(4-Methylphenyl)sulphonyl]-1,2,3,4-tetrahydroisoquinolin-3-yl}methyl 4-methylbenzenesulphonate

To a solution of commercially available [(3S)-1,2,3,4-tetrahydroisoquinolin-3-yl]methanol (30.2 g, 185 mmol) in 750 mL of dichloromethane there are successively added tosyl chloride (91.7 g, ,481 mmol) and then, dropwise, N,N,N-triethylamine (122 mL, 740 mmol). The reaction mixture is stirred at room temperature for 20 hours, diluted with dichloromethane, washed successively with 1M HCl solution, saturated aqueous NaHCO₃ solution and then brine until neutral. The organic phase is then dried over MgSO₄, filtered and concentrated to dryness. The solid obtained is dissolved in a minimum volume of dichloromethane and then cyclohexane is added until a precipitate is formed. This precipitate is then filtered off and washed with cyclohexane. After drying, the title product is obtained in the form of a white crystalline powder.

¹H NMR: 8 (400 MHz; dmso-d6; 300K): 7.75 (d, 2H); 7.6 (d, 2H); 7.5 (d, 2H); 7.3 (d, 2H); 7.15-6.9 (m, 4H); 4.4-4.15 (dd, 2H); 4.25 (m, 1H); 4.0-3.8 (2dd, 2H); 2.7 (2dd, 2H); 2.45 (s, 3H); 2.35 (s, 3H).

Step 5: (3S)-3-(iodomethyl)-2-(p-tolylsulfonyl)-3,4-dihydro-]H-isoquinoline

To a suspension of the compound obtained in Step 4 (4 g, 8.5 mmol) in acetonitrile (10 mL) is added sodium iodide (1.4 g, 9.3 mmol). The reaction mixture is heated under microwave irradiations (100W for 6h), cooled to room temperature. The suspension is filtered. The solid is washed with dichloromethane. The filtrate and the washings are pooled together and concentrated to dryness. The crude is purified by chromatography over silica gel using cyclohexane and ethyl acetate as eluants, the expected product is obtained as a white powder.

¹H NMR: 8 (400 MHz; dmso-d6; 300K): 7.64 (d, 2H), 7.28 (d, 2H), 7.15-7 (m, 4H), 4.5-4.3 (2d, 2H), 4.14 (m, 1H), 3.22 (m, 2H), 2.82 (m, 2H), 2.31 (s, 3H).

MS (ESI): m/z 427 [M]⁺.

Step 6: diethyl 2-[[(3R)-2-(p-tolylsulfonyl)-3,4-dihydro-]H-isoquinolin-3-yl]methyl]propanedioate

To a suspension of sodium hydride (442 mg, 11 mmol) in THF (8 mL) is added dropwise diethyl malonate (1.5 mL, 10 mmol) at room temperature. After 15 minutes, a solution of the compound obtained in Step 5 (4.2 g, 10 mmol) in THF (10 mL) is added dropwise. After 20 minutes at room temperature, the reaction mixture is added in microwave reactor (100° C.-275W) during 19 h. After cooling, the reaction mixture is poured into saturated aqueous ammonium chloride solution, extracted three times with dichloromethane. The organic phase is washed with brine, dried over MgSO4 and concentrated to dryness. The crude is purified by chromatography over silica gel using cyclohexane and ethyl acetate as eluants, affording the desired compound.

¹H NMR: 6 (400 MHz; dmso-d6; 300K): 7.6 (d, 2H), 7.3 (d, 2H), 7.1 (m, 4H), 4.6/4.25 (2d, 2H), 4.2 (m, 1H), 4.1 (m, 4H), 3.55 (t, 1H), 2.6 (m, 2H), 2.3 (s, 3H), 1.85 (2m, 2H), 1.2 (m, 6H).

### [865]Step 7: 3-[(3R)-2-(p-tolylsulfonyl)-3,4-dihydro-]H-isoquinolin-3-yl]propanoic acid

To a solution of the compound obtained in Step 6 (1 g, 2.2 mmol) in a mixture ethanol (8 mL) and water (5 mL) is added lithium hydroxide monohydrate (0.23 g, 5.4 mmol). The reaction mixture is heated to 85° C. overnight. After cooling, the ethanol is evaporated. The aqueous reaction mixture is diluted with water (10 mL) and a solution of 1M HCl is added to reach pH=3. The reaction mixture is extracted with ethyl acetate twice. The organic phase is washed with brine, dried over MgSO₄ and concentrated to dryness. The crude material is diluted with DMSO (10 mL) and a solution of sodium chloride (0.25 g, 4.2 mmol) in water (1 mL). The reaction mixture is heated to 140° C. for 1 h, then cooled to room temperature and diluted with ethyl acetate. The organic layer is separated, dried over MgSO₄ and concentrated to dryness, affording the desired compound.

¹H NMR: 8 (400 MHz; dmso-d6; 300K): 12.15 (m, 1H), 7.65 (d, 2H), 7.3 (d, 2H), 7.15-7 (2m, 4H), 4.6/4.29 (2d, 2H), 4.2 (m, 1H), 2.6 (2dd, 2H), 2.35 (s, 3H), 2.25 (t, 2H), 1.52 (quad, 2H).

### Step 8: N,N-dimethyl-3-[(3R)-2-(p-tolylsulfonyl)-3,4-dihydro-]H-isoquinolin-3-yl]propenamide

To a solution of the compound obtained in Step 7 (10 g, 27.8 mmol) in dichloromethane (50 mL) are added N-(3-Dimethylaminopropyl)-N′-ethylcarbodiimide hydrochloride (5.4 g, 28 mmol), 1-hydroxybenzotriazole hydrate (3.8 g, 28 mmol), N,N-diisopropylethylamine (6.9 mL, 41 mmol) and a solution of 2 M dimethylamine in THF (20.8 mL). The reaction mixture is stirred overnight at room temperature, diluted with dichloromethane, washed successively with water, aqueous 1M HCl solution and brine. The organic phase is dried over MgSO₄, concentrated to dryness and purified by chromatography over silica gel using cyclohexane and ethyl acetate as eluants, affording the desired compound.

¹H NMR: 8 (400 MHz; dmso-d6; 300K): 7.68 (d, 2H), 7.31 (d, 2H), 7.15-7 (m, 4H), 4.65/4.25 (2d, 2H), 4.19 (m, 1H), 2.88/2.78 (2s, 6H), 2.68/2.59 (2dd, 2H), 2.33 (s, 3H), 2.25 (t, 2H), 1.5 (quad, 2 H).

Step 9: N,N-dimethyl-3-[(3R)-2-(p-tolylsulfonyl)-3,4-dihydro-]H-isoquinolin-3-yl]propan-l-amine

To a solution of Step 8 (6.6 g, 17.1 mmol) in anhydrous THF (70 mL) is added dropwise under inert atmosphere a solution of 1M borane tetrahydrofuran complex in tetrahydrofuran (69 mL, 69 mmol). The reaction mixture is heated to 70° C. overnight and cooled to room temperature. The reaction mixture is diluted with dichloromethane and water. The organic phase is washed successively with an aqueous 1M HCl solution, a saturated aqueous NaHCO₃ solution, brine, and dried over MgSO₄ and concentrated. The crude mixture is purified by chromatography over silica gel using dichloromethane and NH₃ 2M in ethanol as eluants, affording the desired compound.

¹H NMR: 6 (400 MHz; dmso-d6; 300K): 7.66 (d, 2H), 7.32 (d, 2H), 7.16-7 (m, 4H), 4.63/4.21 (dd, 2H), 4.15 (m, 1H), 2.67/2.55 (2dd, 2H), 2.33 (s, 3H), 2.1 (t, 2H), 2.04 (s, 6H), 1.35/1.27 (2m, 4H).

### Step 10: N,N-dimethyl-3-[(3R)-1,2,3,4-tetrahydroisoquinolin-3-yl]propan-1-amine

To a solution of naphthalene (13 g, 103 mmol) in anhydrous THF (35 mL) under argon atmosphere is added sodium (2.4 g, 103 mmol) portionwise. After stirring 1 h at room temperature, the reaction mixture is cooled to −78° C. and a solution of Step 9 (4.8 g, 12.2 mmol) in THF (35 mL) is added dropwise. After 3 h of stirring at −78° C., the reaction mixture is allowed to warm to 0° C. and a solution of saturated aqueous ammonium chloride is carefully added (3 mL). The reaction mixture is allowed to warm to room temperature and is evaporated to dryness. The crude mixture is purified by chromatography over silica gel using dichloromethane and NH₃ 2M in ethanol as eluants, affording the desired compound.

¹H NMR: 8 (400 MHz; dmso-d6; 300K): 7.17-7.04 (m, 4H), 4 (s, 2H), 2.92 (m, 1H), 2.82/2.53 (2dd, 2H), 2.32 (m, 2H), 2.22 (s, 6H), 1.65-1.5 (m, H).

### Step 11: ethyl 5-[5-chloro-2-[(3R)-3-[3-(dimethylamino)propyl]-3,4-dihydro-]H-isoquinoline-2-carbonyl]phenyl]-1,2-dimethyl-pyrrole-3-carboxylate

To a solution of the compound obtained in Step 3 (15.5 g, 48 mmol) in dichloromethane (3L) are added N-(3-dimethylaminopropyl)-N′-ethylcarbodiimide hydrochloride (9.2 g, 48 mmol), 1-hydroxybenzotriazole hydrate (7.4 g, 48 mmol), the compound obtained in Step 10 (10 g, 48 mmol) and N-ethyl-N-isopropyl-propan-2-amine (28 mL, 160 mmol). The reaction mixture is stirred for 3 h at room temperature, diluted with dichloromethane, washed with water, concentrated to dryness and purified by chromatography over silica gel using dichloromethane and NH₃ 2M in ethanol as eluants, affording the desired compound.

¹H NMR: 8 (500 MHz; dmso-d6; 300K): 7.6-7.25 (m, 3H), 7.2-6.85 (m, 4H), 6.55-6.15 (5s, 1H), 5.35-3.75 (m, 2H), 4.85/4.75/3.6/3.55 (4m, 1H), 4.1 (m, 2H), 3.45/3.2 (2s, 3H), 3-1.8 (m, 2H), 2.5-2 (3s, 3H), 2.2-1.8 (m, 2H), 2.05-1.9 (4s, 6H), 1.4/1.I/O.9 (3m, 2H), 1.4/1.25/1.1 (m, 2H), 1.2/1.1 (2t, 3H).

MS (ESI): m/z 521 [M+H]+.

### Step 12: 5-[5-chloro-2-[(3R)-3-[3-(dimethylamino)propyl]-3,4-dihydro-]H-isoquinoline-2-carbonyl]phenyl]-1,2-dimethyl-pyrrole-3-carboxylic acid

To a solution of the compound obtained in Step 11 (400 mg, 0.76 mmol) in methanol (2 mL) is added a solution of lithium hydroxide monohydrate (112 mg, 2.6 mmol) in water (2 mL). The reaction mixture is heated at reflux overnight then partially concentrated. Dichloromethane and 1M HCl solution are added to the reaction mixture. The organic phase is separated, dried over MgSO₄ and concentrated to dryness. The residue is purified by chromatography over silica gel using dichloromethane and methanol as eluants, affording the desired compound.

MS (ESI): m/z 494 [M]+.

### Step 13: N-[4-[tert-butyl(dimethyl)silyl]oxyphenyl]-5-[5-chloro-2-[(3R)-3-[3-(dimethylamino)propyl]-3,4-dihydro-1H-isoquinoline-2-carbonyl]phenyl]-N-(5-cyano-1,2-dimethyl-pyrrol-3-yl)-1,2-dimethyl-pyrrole-3-carboxamide

To a solution of the compound obtained in Step 12 (480 mg, 0.97 mmol) in 1,2-dichloroethane (60 mL) is added 1-chloro-N,N,2-trimethyl-prop-1-en-1-amine (170 μL, 1.3 mmol). The reaction is stirred at room temperature overnight. To the reaction are added successively 4-[4-[tert-butyl(dimethyl)silyl]oxyanilino]-1,5-dimethyl-pyrrole-2-carbonitrile (500 mg, 1.5 mmol prepared using the procedure described in WO2015/011400, Preparation 18”), pyridine (0.5 mL, 5.8 mmol) and 1,2-dichloroethane (60 mL). The reaction mixture is heated to reflux for 2h, cooled to room temperature, filtrated on Celite®, concentrated to dryness and purified by chromatography over silica gel using dichloromethane and NH₃ 2M methanol as eluants, affording the desired compound.

¹H NMR: 8 (500 MHz; dmso-d6; 300K): 7.55-6.9 (m, 7H), 6.9-6.6 (m, 4H), 6.8-6.5 (m, 1H), 5.45-5.1 (m, 1H), 5.35-4 (m, 2H), 4.8-3.5 (m, 1H), 3.7-3.1 (m, 6H), 2.75-2.5 (m, 2H), 2.45-1.8 (m,6 H), 2.3-1.9 (m, 2H), 2.15-1.95 (m, 6H), 1.45-1.3 (m, 4H), 0.85 (s, 9H), 0.15 (s, 6H).

¹³C NMR: 8 (500 MHz; dmso-d6; 300K): 130.9-124, 127.6, 119.7, 116.9, 110.5/109.5, 56.2, 51.9/46.7/45.3, 44.1/40.1, 42, 33, 31.8, 31.5/31.1, 26.8/20.4, 25.6, 11.4, 9.5,-4.5. 77

### Step 14: 5-[5-chloro-2-[(3R)-3-[3-(dimethylamino)propyl]-3,4-dihydro-]H-isoquinoline-2-carbonyl]phenyl]-N-(5-cyano-1,2-dimethyl-pyrrol-3-yl)-N-(4-hydroxyphenyl)-],2-dimethyl-pyrrole-3-carboxamide

To a solution of the compound obtained in Step 13 (0.5 g, 0.61 mmol) in THF (20 mL) is added at room temperature a solution of 1M tetra-n-butylammonium fluoride in THF (1 mL, 1 mmol). The reaction mixture is stirred for 4 h at room temperature and diluted with dichloromethane and a saturated aqueous solution of NaHCO₃. The organic phase is separated, washed successively with water, brine, then dried over MgSO₄ and concentrated. The crude is purified by chromatography over silica gel using dichloromethane and NH₃ 2M methanol as eluants, affording the desired compound.

¹H NMR: 8 (500 MHz; dmso-d6; 300K): 9.35 (broad s, 1H), 7.55-6.85 (m, 7H), 6.95/6.75 (2d, 2H), 6.75-6.5 (m, 1H), 6.65-6.55 (m, 2H), 5.8-5.25 (m, 1H), 5.3-3.6 (m, 2H), 4.8/4.75/3.45 (3m, 1H), 3.65-3.5 (m, 3H), 3.45-3.05 (m, 3H), 2.65-1.65 (m, 2H), 2.4-1.95 (m, 3H), 2.25-1.75 (m, 2H), 2.15-1.75 (m, 3H), 2.1-1.95 (4s, 6H), 1.45-0.6 (m, 4H).

¹³C NMR: 8 (500 MHz; dmso-d6; 300K): 130-126, 128, 117, 116, 111-110, 59, 53/47/46, 45-40, 45, 33, 32-31, 32, 29-24, 12, 10.

HRMS (ESI): [M+H]+=703.3155 (703.3158 calculated).

### Preparation of P3: N-[1-(difluoromethyl)pyrazol-4-yl]-5-[2-[2-[4-(2-morpholinoethoxy) phenyl]acetyl]-7- [(3R)-3-methyl-3,4-dihydro-1H-isoquinoline-2-carbonyl]-3,4-dihydro-1H-isoquinolin-6-yl]-N-(4-hydroxyphenyl)-1,2-dimethyl-pyrrole-3-carboxamide

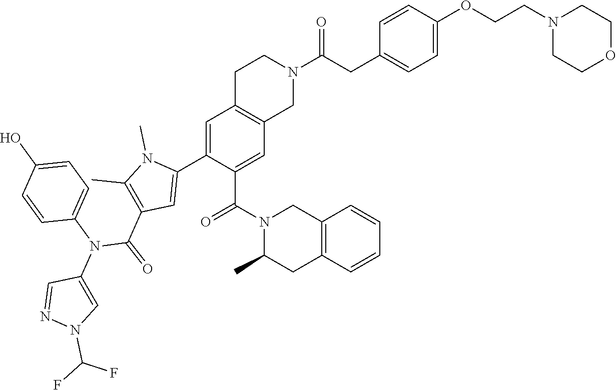 [SMILES: Cc1c(C(=O)N(c2ccc(O)cc2)c2cnn(C(F)F)c2)cc(-c2cc3c(cc2C(=O)N2Cc4ccccc4C[C@H]2C)CN(C(=O)Cc2ccc(OCCN4CCOCC4)cc2)CC3)n1C]

### Step A: N-[4-[tert-butyl(dimethyl)silyl]oxyphenyl]-]-(difluoromethyl)pyrazol-4-amine

Using General procedure 1b, starting from 4-bromo-]-(difluoromethyl)pyrazole (1.00 g, 5.08 mmol) as the appropriate aryl-bromide and Preparation IIIa (1.19 g, 1.05 eq.) as the appropriate aniline, afforded the title compound (1.19 g, 69%).

¹H NMR (500 MHz, DMSO-d6) δ ppm 7.99 (s, 1H), 7.72 (s, 1H), 7.67 (t, 1H), 7.63 (s, 1H), 6.79 (d, 2H), 6.70 (d, 2H), 0.93 (s, 9H), 0.14 (s, 6H); ¹³C NMR (125 MHz, DMSO-d6) δ ppm 147.7, 139.7, 136.3, 129.6, 120.8, 115.5, 115.4, 111.3, 26.1, 18.4, -4.1.

HRMS-ESI (m/z) [M+H]⁺ calcd for C₁₆H₂₄F₂N₃OSi: 340.1651, found 340.1650.

### Step B: 9H-fluoren-9-ylmethyl 6-[4-[[4-[tert-butyl(dimethyl)silyl]oxyphenyl]-[ ]-(difluoromethyl)pyrazol-4-yl]carbamoyl]-1,5-dimethyl-pyrrol-2-yl]-7-[(3R)-3-methyl-3,4-dihydro-]H-isoquinoline-2-carbonyl]-3,4-dihydro-]H-isoquinoline-2-carboxylate

Using the procedure described in Preparation VIIb Step C and the product from Step A (1.16 g, 3.42 mmol) as the appropriate aniline instead of the product of Preparation IVa Step B afforded the title product (2.18 g, 77%).

¹H NMR (500 MHz, DMSO-d6) δ ppm 8.3-6.53 (m, 21H), 5.34-4.7 (s, 1H), 5.33-2.07 (m, 14 H), 3.39-3.03 (s, 3H), 2.48-2.1 (s, 3H), 1.12-0.48 (brd, 3H), 0.86-0.75 (s, 9H), 0.12-0 (s, 6H).

HRMS-ESI (m/z) [M+H]⁺ calcd for C₅sH₆₁F₂N₆O₅Si: 987.4435, found 987.4438.

### Step C: N-[4-[tert-butyl(dimethyl)silyl]oxyphenyl]-N-[ ]-(difluoromethyl)pyrazol-4-yl]-1,2-dimethyl-5-[7-[(3R)-3-methyl-3,4-dihydro-]H-isoquinoline-2-carbonyl]-1,2,3,4-tetrahydroisoquinolin-6-yl]pyrrole-3-carboxamide

Using the procedure described in Preparation VIIb Step D and the product from Step B (2.18 g, 2.21 mmol) instead of the product from Preparation VIIb Step C afforded the title product (0.83 g, 49%).

¹H NMR (500 MHz, DMSO-d6) δ ppm 8.24-8.02 (s, 1H), 7.73 (t, 1H), 7.54-7.37 (s, 1H), 7.26-6.55 (m, 10H), 5.3-4.67 (s, 1H), 5.26-2.09 (m, 12H), 3.35-3.02 (s, 3H), 2.46-2.08 (s, 3H), 1.04-0.52 (d, 3H), 0.86 (s, 9H), 0.17-0.04 (s, 6H).

HRMS-ESI (m/z) [M+H]⁺ calcd for C₄₃H₅₁F₂N₆O₃Si: 765.3754, found 765.3756.

### Step D: N-[4-[tert-butyl(dimethyl)silyl]oxyphenyl]-N-[ ]-(difluoromethyl)pyrazol-4-yl]-1,2-dimethyl-5-[7-[(3R)-3-methyl-3,4-dihydro-]H-isoquinoline-2-carbonyl]-2-[2-[4-(2-morpholinoethoxy)phenyl]acetyl]-3,4-dihydro-]H-isoquinolin-6-yl]pyrrole-3-carboxamide

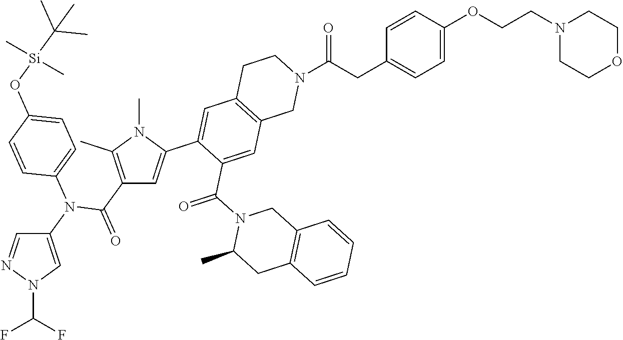 [SMILES: Cc1c(C(=O)N(c2ccc(O[Si](C)(C)C(C)(C)C)cc2)c2cnn(C(F)F)c2)cc(-c2cc3c(cc2C(=O)N2Cc4ccccc4C[C@H]2C)CN(C(=O)Cc2ccc(OCCN4CCOCC4)cc2)CC3)n1C]

Using modified General procedure 4a (using TBTU instead of HATU and omitting preparative HPLC flash chromatography), starting from the product of Preparation Va (283 mg, 1.2 eq.) as the appropriate carboxylic acid and the product from Step C (680 mg, 0.889 mmol) as the appropriate amine, afforded the title compound (643 mg, 71%).

¹H NMR (500 MHz, DMSO-d6) δ ppm 8.22-8.04 (s, 1H), 7.72 (t, 1H), 7.53-7.36 (s, 1H), 7.26-6.58 (m, 14H), 5.29-2.08 (m, 13H), 5.28-4.73 (s, 1H), 4.05 (t, 2H), 3.56 (m, 4H), 3.35-3.01 (s, 3H), 2.66 (t, 2H), 2.47-2.09 (s, 3H), 2.45 (m, 4H), 1.04-0.49 (d, 3H), 0.89-0.8 (s, 9H), 0.13-0.01 (s, 6H).

HRMS-ESI (m/z) [M+H]⁺ calcd for Cs₇H₆₈F₂N₇06Si: 1012.4963, found 1012.4968.

### Step E: N-[ ]-(difluoromethyl)pyrazol-4-yl]-5-[2-[2-[4-(2-morpholinoethoxy) phenyl]acetyl]-7-[(3R)-3-methyl-3,4-dihydro-]H-isoquinoline-2-carbonyl]-3, 4-dihydro-]H-isoquinolin-6-yl]-N-(4-hydroxyphenyl)-1,2-dimethyl-pyrrole-3-carboxamide

To the product from Step D (100 mg, 0.099 mmol) dissolved in acetonitrile (150 mL/mmol) 2M aq. NaOH solution (6 eq.) was added and the resulting mixture was stirred at rt. After reaching complete conversion the pH was adjusted to 7 by 2M aq. HCl solution and the product was partitioned between DCM and water. The phases were separated, and the organic phase was dried over MgSO₄ and concentrated in vacuo. The crude material was purified by automated flash chromatography using DCM and MeOH as eluents to afford the title compound (19.5 mg, 22%).

HRMS-ESI (m/z) [M+H]⁺ calcd for Cs₁H₅₄F₂N₇O6: 898.4104, found 898.4152.

### Preparation of P4: N-(5-cyano-1,2-dimethyl-1H-pyrrol-3-yl)-N-(4-hydroxyphenyl)-1,2-dimethyl-5-{7-[(3R)-3-methyl-1,2,3,4-tetrahydroisoquinoline-2-carbonyl]-2-(2-{4-[2-(morpholin-4-yl)ethoxy]phenyl}acetyl)-1,2,3,4-tetrahydroisoquinolin-6-yl}-1H-pyrrole-3-carboxamide

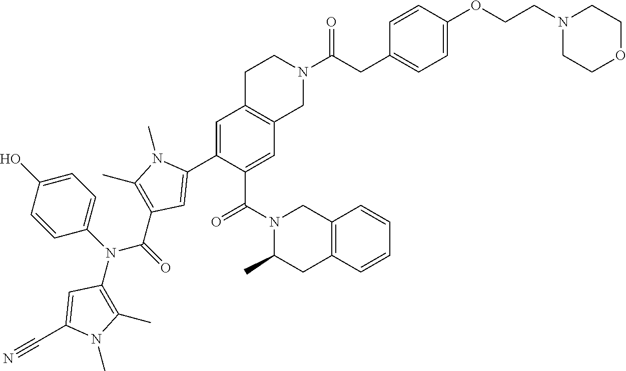 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(O)cc2)cc(C#N)n1C]

### Step A: N-[4-[tert-butyl(dimethyl)silyl]oxyphenyl]-N-(5-cyano-1,2-dimethyl-pyrrol-3-yl)-1,2-dimethyl-5-[7-[(3R)-3-methyl-3,4-dihydro-]H-isoquinoline-2-carbonyl]-2-[2-[4-(2-morpholinoethoxy)phenyl]acetyl]-3,4-dihydro-]H-isoquinolin-6-yl]pyrrole-3-carboxamide

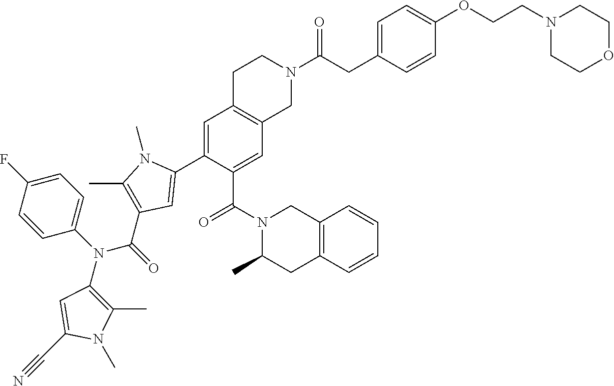 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccc(F)cc2)cc(C#N)n1C]

Using the procedure described in Step D of the Preparation of P3, starting from the product of Preparation Va (1.27 g, 1.5 eq.) as the appropriate carboxylic acid and Preparation VIIb (2.45 g, 3.194 mmol) as the appropriate amine, afforded the title compound (2.06 g, 64%).

HRMS-ESI (m/z) [M+H]⁺ calcd for C₆₀H₇₂N₇O₆Si: 1014.5308, found 1014.5312.

### Step B: N-(5-cyano-1,2-dimethyl-]H-pyrrol-3-yl)-N-(4-hydroxyphenyl)-1,2-dimethyl-5-[7-[(3R)-3-methyl-1,2,3,4-tetrahydroisoquinoline-2-carbonyl]-2-(2-[4-[2-(morpholin-4-yl)ethoxy]phenyl}acetyl)-1,2,3,4-tetrahydroisoquinolin-6-yl}-]H-pyrrole-3-carboxamide

According the procedure described in Step E of the Preparation of P3, the product from Step A (390 mg, 0.385 mmol) was treated with 2M aq. NaOH solution to afford the title compound (299 mg, 86%).

HRMS-ESI (m/z) [M+H]⁺ calcd for C₅₄H₅₁N₇O₆: 900.4443, found 900.4445.

### Example 2. Synthesis and Characterization of Linkers, Linker-Payload, and Precursors Thereof

Exemplary linkers, linker-payloads, and precursors thereof were synthesized using exemplary methods described in this example.

### Materials, Methods & General Procedures

All reagents obtained from commercial sources were used without further purification.

Anhydrous solvents were obtained from commercial sources and used without further drying. Flash chromatography was performed on CombiFlash Rf (Teledyne ISCO) with pre-packed silica-gel cartridges (Macherey-Nagel Chromabond Flash). Thin layer chromatography was conducted with 5×10 cm plates coated with Merck Type 60 F254 silica-gel. Microwave heating was performed in CEM Discover® instrument.

NMR data were acquired at a temperature of 298K on a Bruker Avance NMR spectrometer equipped with a 5 mm BBFO CryoProbe with z-gradient operating at a frequency of 400.13 MHz for ¹H, 376.50 MHz for ¹⁹F, 100.61 MHz for ¹³C. Chemical shifts for the ¹H and ¹³C spectra were referenced by setting internal tetramethylsilane (TMS) to 0 ppm.

Analytical Methods
LC/MS data was acquired using an
instrument with the following parameters:

| | |
|---|---|
| Pump | Waters AcQuity UPLC Binary Solvent Manager |
| Sample Manager | Waters AcQuity UPLC Sample Manager |
| Column Compartment | Waters AcQuity UPLC Column Manager |
| Detector | Waters AcQuity UPLC PDA |
| ELSD | Shimadzu ELSD-LTII |
| Mass Spec | Waters SQD |
| Columns | AcQuity UPLC BEH C18 1.7 μm 2.1 × 50 mm |
| Eluent A1 | 0.1% Formic Acid in Water |
| Eluent B1 | 0.1% Formic Acid in Acetonitrile |
| Eluent A2 | 5 mM Ammonium Hydroxide in Water |
| Eluent B2 | 5 mM Ammonium Hydroxide in Acetonitrile |

The methods used to generate LC/MS data were as follows:

| Eluent A1 | 0.1% Formic Acid in Water | | |
|---|---|---|---|
| Eluent B1 | 0.1% Formic Acid in Acetonitrile | | |
| Flow | 1.0 mL/min | | |
| Stop Time | 3.00 min | | |
| pH | 2.6 | | |
| Gradient | Time | % A (Eluent A1) | % B (Eluent B1) |
| | 0 | 95 | 5 |
| | 0.2 | 95 | 5 |
| | 2 | 5 | 95 |
| | 2.5 | 5 | 95 |
| | 2.6 | 95 | 5 |
| | 3 | 95 | 5 |

| | |
|---|---|
| Column | AcQuity UPLC BEH C18 1.7 μm 2.1 × 50 mm |
| Column | 50° C. |
| Temperature | |
| TAC | 210-400 nm |
| Mass Range | 120-1500 Da |
| Scan Time | 0.3 sec |

2 min acidic method:
2 min basic method:

| Eluent A1 | 5 mM Ammonium Hydroxide in Water | | |
|---|---|---|---|
| Eluent B1 | 5 mM Ammonium Hydroxide in Acetonitrile | | |
| Flow | 1.0 mL/min | | |
| Stop Time | 3.00 min | | |
| pH | 10.2 | | |
| Gradient | Time | % A (Eluent A1) | % B (Eluent B1) |
| | 0 | 95 | 5 |
| | 0.2 | 95 | 5 |
| | 2 | 5 | 95 |
| | 2.5 | 5 | 95 |
| | 2.6 | 95 | 5 |
| | 3 | 95 | 5 |

| | |
|---|---|
| Column | AcQuity UPLC BEH C18 1.7 μm 2.1 × 50 mm |
| Column | 50° C. |
| Temperature | |
| TAC | 210-400 nm |
| Mass Range | 120-1500 Da |
| Scan Time | 0.3 sec |

5 min acidic method

| Flow | 1.0 mL/min | | |
|---|---|---|---|
| Stop Time | 5.20 min | | |
| pH | 2.6 | | |
| Gradient | Time | % A (Eluent A1) | % B (Eluent B1) |
| | 0 | 98 | 2 |
| | 4.4 | 2 | 98 |
| | 5.15 | 2 | 98 |
| | 5.19 | 98 | 2 |
| Column | AcQuity UPLC BEH C18 1.7 μm 2.1 × 50 mm | | |
| Column | 50° C. | | |
| Temperature | | | |

| | |
|---|---|
| TAC | 210-400 nm |
| Mass Range | 120-1500 Da |
| Scan Time | 0.3 sec |

HRMS data was acquired using an instrument with the following parameters: HRMS_QT01

| | |
|---|---|
| Pump | Waters AcQuity UPLC Binary Solvent Manager |
| Sample Manager | Waters AcQuity UPLC Sample Manager |
| Column | Waters AcQuity UPLC Column Manager |
| Compartment | |
| Detector | Waters AcQuity UPLC PDA |
| ELSD | n/a |
| Mass Spec | Waters Xevo G2 Qtof |
| Columns | AcQuity UPLC PrST C4 300 Å 1.7 μm 2.1 × 100 mm |
| | AcQuity UPLC CSH C18 1.7 μm 2.1 × 50 mm |
| | ProSwift RP-3U 4.6 × 50 mm SS |
| Eluent A1 | 0.1% Formic Acid in Water |
| Eluent B1 | 0.1% Formic Acid in Acetonitrile |
| Eluent A2 | 0.05% Triflouroacetic Acid in Water |
| Eluent B2 | 0.05% Triflouroacetic Acid in Acetonitrile |

### HRMS_QT02

| | |
|---|---|
| Pump | Waters AcQuity UPLC Binary Solvent Manager |
| Sample Manager | Waters AcQuity UPLC Sample Manager |
| Column | Waters AcQuity UPLC Column Manager |
| Compartment | |
| Detector | Waters AcQuity UPLC PDA |
| ELSD | n/a |
| Mass Spec | Waters Xevo G2 Qtof |
| Columns | AcQuity UPLC PrST C4 300 Å 1.7 μm 2.1 × 100 mm |
| | AcQuity UPLC CSH C18 1.7 μm 2.1 × 50 mm |
| | POROS R1 10 μm 2.1 × 100 mm |
| Eluent A1 | 0.1% Formic Acid in Water |
| Eluent B1 | 0.1% Formic Acid in 80:20 |
| | Isopropanol:Acetonitrile |
| Eluent A2 | 0.05% Triflouroacetic Acid in Water |
| Eluent B2 | 0.05% Triflouroacetic Acid in Acetonitrile |

The method used to generate HRMS data for linker/payloads and synthetic intermediates was as follows:

Peptide_300-4000_Da_5 min QT1

| Flow | 1.0 mL/min | | |
|---|---|---|---|
| Stop Time | 5.2 min | | |
| pH | 2.6 | | |
| Gradient | Time | % A (Eluent | % B (Eluent B2) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 1.7 μm 2.1 × 50 mm |
| Column Temp | 50° C. |
| TAC | 210-400 nm |
| Mass Range | 300-4000 Da |
| Processing Range | n/a |
| Scan Time | 0.5 sec |

Peptide_300-10000_Da_5 min QT1

| Flow | 1.0 mL/min | | |
|---|---|---|---|
| Stop Time | 5.2 min | | |
| pH | 2.6 | | |
| Gradient | Time | % A (Eluent A2) | % B (Eluent B2) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 1.7 μm 2.1 × 50 mm |
| Column Temp | 50° C. |
| TAC | 210-400 nm |
| Mass Range | 300-4000 Da |
| Processing Range | 300-10000 Da |
| Scan Time | 0.5 sec |

Peptide_300-4000_Da_5 min QT2

| Flow | 1.0 mL/min | | |
|---|---|---|---|
| Stop Time | 5.2 min | | |
| pH | 2.6 | | |
| Gradient | Time | % A (Eluent A2) | % B (Eluent B2) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 1.7 μm 2.1 × 50 mm |
| Column Temp | 80° C. |
| TAC | 210-400 nm |
| Mass Range | 300-4000 Da |
| Processing Range | n/a |
| Scan Time | 0.2 sec |

Peptide_300-10000_Da_5 min QT2

| Flow | 1.0 mL/min | | |
|---|---|---|---|
| Stop Time | 5.2 min | | |
| pH | 2.6 | | |
| Gradient | Time | % A (Eluent | % B (Eluent B2) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 1.7 μm 2.1 × 50 mm |
| Column Temp | 80° C. |
| TAC | 210-400 nm |
| Mass Range | 300-4000 Da |
| Processing Range | 300-10000 Da |
| Scan Time | 0.2 sec |

### Preparative RP-HPLC:

Preparative-HPLC (“Prep-HPLC”) data were acquired using Teledyne ISCO purification systems using C18 or C4 RP ISCO or ISCO-gold columns.

Four Prep-HPLC methods were used:
a. TFA method: solvent: A water+0.05% TFA, B acetonitrile+0.05% TFA, gradient from 5 to 100% B in 15 to 30 CV
b. NH₄HCO₃ method: solvent: A water+0.02 M NH₄HCO₃, B acetonitrile/water 80/20+0.02 M NH₄HCO₃, gradient from 5 to 100% B in 15 to 30 CV
c. Neutral method: solvent: A water, B acetonitrile, gradient from 5 to 100% B in 15 to 30 CV
d. Formic Acid method: solvent: A water+0.05% Formic Acid, B acetonitrile+0.05% Formic Acid, gradient from 5 to 100% B in 15 to 30 CV Gradient variations of methods a.-d. were employed as appropriate. All the fractions containing the pure compound were combined and directly freeze-dried to afford the compound as an amorphous powder.

### Synthesis of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate

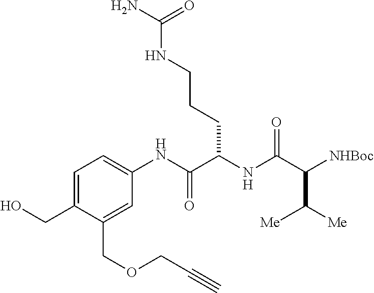 [SMILES: C#CCOCc1cc(NC(=O)[C@H](CCCNC(N)=O)NC(=O)[C@@H](NC(=O)OC(C)(C)C)C(C)C)ccc1CO]

### Step 1: Synthesis of 2-(bromomethyl)-4-nitrobenzoic acid

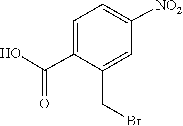 [SMILES: O=C(O)c1ccc([N+](=O)[O-])cc1CBr]

To a stirred solution of 2-methyl-4-nitrobenzoic acid (300 g, 1.5371 mol) in CCl₄ (3000 mL) was added NBS (300.93 g, 1.6908 mol) and AIBN (37.86 g, 0.2305 mol) at rt. The reaction mixture was stirred at 80° C. for 16h. Reaction mixture was monitored by TLC analysis. The reaction mixture was diluted with sat. NaHCO₃ solution (2 lit) and extracted with ethyl acetate (2×2 lit). The combined organic layer was dried over anhydrous sodium sulphate and concentrated under reduced pressure. The crude compound was purified by column chromatography on silica gel using 2-3% of ethylacetate in petroleum ether as an eluent and 2-(bromomethyl)-4-nitrobenzoic acid was obtained (250 g, 59% yield). ¹H NMR (400 MHz, CDCl3): δ 8.35 (d, J=2.0 Hz, 1H), 8.20 (q, J=8.8, 2.4 Hz, 1H), 8.12 (d, J=8.8 Hz, 1H), 4.97 (s, 2H), 4.00 (s, 3H).

### Step 2: Synthesis of 4-nitro-2-((prop-2-yn-1-yloxy)methyl)benzoic acid

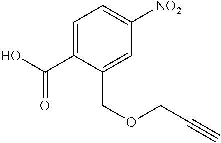 [SMILES: C#CCOCc1cc([N+](=O)[O-])ccc1C(=O)O]

To the mixture of 2-(bromomethyl)-4-nitrobenzoic acid (250 g, 0.9122 mol) in ACN (5000 mL) was added prop-2-yn-1-ol (255.68 g, 265.50 mL, 4.5609 mol, d=0.963 g/mL) and Cs₂CO₃ (743.03 g, 2.2805 mol) at rt. The resulting mixture was heated to 80° C. for 16 h. The reaction mixture was filtered through celite pad washed with ethylacetate (2 lit). The filterate was concentrated under reduced pressure. The obtained crude compound was added sat. NaHCO₃ solution (1 lit) and the aq layer was acidified to pH 2 by using 2N HCl (2 lit). After filtration vacuum drying 4-nitro-2-((prop-2-yn-1-yloxy)methyl)benzoic acid was obtained (130 g, 60.6%). H NMR (400 MHz, DMSO): δ 13.61 (brs, 1H), 8.37 (d, J=2.4 Hz, 1H), 8.23 (dd, J=2.4, 8.4 Hz, 1H), 8.10 (d, J=8.8 Hz, 1H), 4.95 (s, 2H), 4.37 (d, J=2.4 Hz, 2H), 3.52 (t, J=2.4 Hz, 1H)

### Step 3: Synthesis of methyl 4-nitro-2-((prop-2-yn-1-yloxy)methyl)benzoate

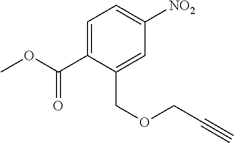 [SMILES: C#CCOCc1cc([N+](=O)[O-])ccc1C(=O)OC]

To a stirred solution of 4-nitro-2-((prop-2-yn-1-yloxy)methyl)benzoic acid (130 g, 0.5527 mol) in MeOH (1300 mL) was added SOCl₂ (526.08 g, 320.78 mL, 4.4219 mol, d=1.64 g/mL) slowly at 0° C. The reaction stirred at 70° C. for 4 h. The reaction solvent was evaporated under reduced pressure. The obtained residue was dissolved in ethylacetate (1000 mL) and washed with sat.NaHCO₃ (600 mL), water (500 mL) and brine solution (500 mL). The separated organic layer was dried over sodium sulphate, filtered and evaporated under reduced pressure to yield methyl 4-nitro-2-((prop-2-yn-1-yloxy)methyl)benzoate (110 g, 80% yield). ¹H NMR (400 MHz, CDCl3): δ 8.56 (t, J=0.8 Hz, 1H), 8.18-8.09 (m, 2H), 5.03 (s, 2H), 4.35 (d, J=2.4 Hz, 2H), 3.96 (s, 3H), 2.49 (t, J=2.4 Hz, 1H).

### Step 4: Synthesis of methyl 4-amino-2-((prop-2-yn-1-yloxy)methyl)benzoate

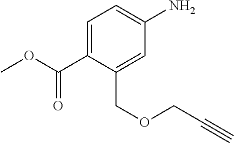 [SMILES: C#CCOCc1cc(N)ccc1C(=O)OC]

To a solution of methyl 4-nitro-2-((prop-2-yn-1-yloxy)methyl)benzoate (110 g, 0.4414 mol) in a mixture of EtOH (1100 mL) and H₂O (550 mL) was added Fe Powder (197.21 g, 3.5310 mol) and NH₄Cl (188.88 g, 3.5310 mol) at rt. The resulting mixture was heated at 80° C. for 16 h. The reaction mixture was cooled to rt and filtered through celite and washed with ethylacetate (2 lit). The filtrate was concentrated under reduced pressure up to half of the volume. To the residue ethylacetate (1.5 lit) was added and separated the two layers and the aqueous layer was extracted with ethyl acetate (2 lit). The combined organic layer was dried over anhydrous sodium sulphate and concentrated under reduced pressure to obtain crude product. Purification by SiO₂ column chromatography (15-20% of ethylacetate in pet-ether) yielded methyl 4-amino-2-((prop-2-yn-1-yloxy)methyl)benzoate (70 g, 72% yield). ¹H NMR (400 MHz, CDCl3): δ 7.67 (d, J=8.8 Hz, 1H), 6.78 (t, J=1.6 Hz, 1H), 6.48 (q, J=8.4, 2.4 Hz, 1H), 4.79 (s, 2H), 4.25 (d, J=2.4 Hz, 2H), 3.70 (d, J=4.0 Hz, 3H), 3.42 (t, J=2.4 Hz, 1H).

### Step 5: Synthesis of (4-amino-2-((prop-2-yn-1-yloxy)methyl)phenyl)methanol

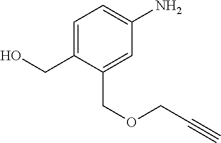

To a stirred solution of THF (1000 mL) was added LiAlH₄ (1 M in THF) (21.23 g, 798.2 mmol, 798.2 mL) slowly at 0° C. A solution of methyl 4-amino-2-((prop-2-yn-1-yloxy)methyl)benzoate (70 g, 319.3 mmol) in THF (800 mL) was added slowly at 0° C. The reaction was stirred at rt for 4 h. The reaction mixture was cooled to 0° C., then was added water (22 mL) very slowly and followed by the addition of 20% NaOH (22 mL) and water (66 mL). The reaction mixture was stirred at 0° C. for 30 min. Anhydrous sodium sulphate was added to absorb excess of water. The mixture was filtered through celite. The filter cake was washed with ethylacetate (1000 mL) and 10% MeOH/DCM (500 mL). The filtrate was concentrated under reduced pressure. The resulting crude compound was purified by SiO₂ column chromatography (35-40% of ethylacetate in pet-ether as an eluent) to give yield (4-amino-2-((prop-2-yn-1-yloxy)methyl)phenyl)methanol (50.6 g, 83% yield). ¹H NMR (400 MHz, CDCl3): δ 6.98 (d, J=8.0 Hz, 1H), 6.56 (d, J=2.4 Hz, 1H), 6.43 (dd, J=2.4, 8.0 Hz, 1H), 4.98 (s, 2H), 4.64 (t, J=5.2 Hz, 1H), 4.47 (s, 2H), 4.34 (d, J=5.6 Hz, 2H), 4.15 (d, J=2.4 Hz, 2H), 3.46 (t, J=2.4 Hz, 1H).

### Step 6: Synthesis of (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate

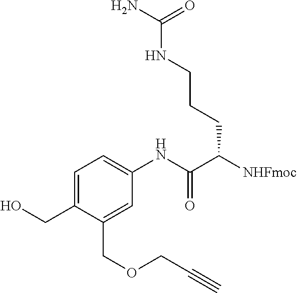

To a solution of (4-amino-2-((prop-2-yn-1-yloxy)methyl)phenyl)methanol (1.92 g, 10.04 mmol, 1.0 equiv.), (9H-fluoren-9-yl)methyl (S)-(1-amino-i-oxo-5-ureidopentan-2-yl)carbamate (3.99 g, 10.04 mmol, 1.0 equiv.), and (1-[bis(dimethylamino)methylene]-1H-1,2,3-triazolo[4,5-b]pyridinium 3-oxid hexafluorophosphate (4.20 g, 11.04 mmol, 1.1 equiv.) in DMF (10 mL) was added N,N-diisopropylethylamine (2.62 mL, 15.06 mmol, 1.5 equiv.). After stirring at ambient temperature for 1 hour, the mixture was poured into water (200 mL). The resulting solids were filtered, rinsed with water, and dried under vacuum, and (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate was obtained (6.08 g, 99%). LCMS: MH+=571.5; Rt=0.93 min (2 min acidic method-Method A).

### Step 7: Synthesis of (S)-2-amino-N-(4-(hydroxymethyl)-3-((prop-2-yn-1-yloxy)methyl)phenyl)-5-ureidopentanamide

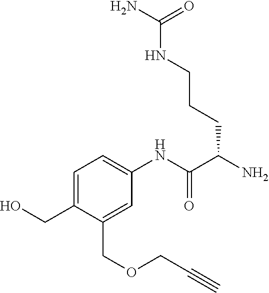 [SMILES: C#CCOCc1cc(NC(=O)[C@@H](N)CCCNC(N)=O)ccc1CO]

To (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate (6.08 g, 10.65 mmol, 1.0 equiv.) was added dimethylamine (2 M in THF, 21.31 mL, 42.62 mmol, 4 equiv.). After stirring at ambient temperature for 1.5 hours, the supernatant solution was decanted from the gumlike residue that had formed. The residue was triturated with ether (3×50 mL) and the resulting solids were filtered, washed with ether, and dried under vacuum. (S)-2-amino-N-(4-(hydroxymethyl)-3-((prop-2-yn-1-yloxy)methyl)phenyl)-5-ureidopentanamide was obtained (3.50 g, 10.04 mmol, 94%). LCMS: MH+349.3; Rt=0.42 min (2 min acidic method-Method A).

### Step 8: Synthesis of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate

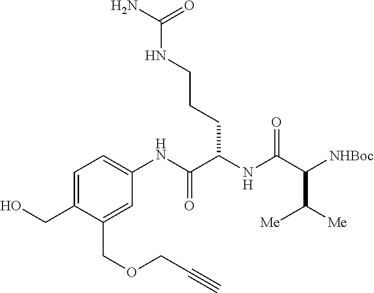 [SMILES: C#CCOCc1cc(NC(=O)[C@H](CCCNC(N)=O)NC(=O)[C@@H](NC(=O)OC(C)(C)C)C(C)C)ccc1CO]

To a solution of (S)-2-amino-N-(4-(hydroxymethyl)-3-((prop-2-yn-1-yloxy)methyl)phenyl)-5-ureidopentanamide (3.50 g, 10.04 mmol, 1.0 equiv.), (tert-butoxycarbonyl)-L-valine (2.62 g, 12.05 mmol, 1.2 equiv.), and (1-[bis(dimethylamino)methylene]-1H-1,2,3-triazolo[4,5-b]pyridinium 3-oxid hexafluorophosphate (4.58 g, 12.05 mmol, 1.2 equiv.) in DMF (10 mL) was added N,N-diisopropylethylamine (3.50 mL, 20.08 mmol, 2.0 equiv). After stirring at ambient temperature for 2 hours, the mixture was poured into water (200 mL) and the resulting suspension was extracted with EtOAc (3×100 mL). The combined organic layers were dried over sodium sulfate and concentrated under vacuum. After purification by ISCO SiO₂ chromatography (0-20% methanol/dichloromethane), tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate was obtained (2.49 g, 4.55 mmol, 45%). 1H NMR (400 MHz, DMSO-d6) δ 10.00 (s, 1H), 7.96 (d, J=7.7 Hz, 1H), 7.55 (dq, J=4.9, 2.2 Hz, 2H, aryl), 7.32 (d, J=8.9 Hz, 1H, aryl), 6.76 (d, J=8.9 Hz, 1H), 5.95 (t, J=5.8 Hz, 1H), 5.38 (s, 2H), 5.01 (t, J=5.5 Hz, 1H), 4.54 (s, 2H), 4.45 (dd, J=25.2, 5.3 Hz, 3H), 4.20 (d, J=2.4 Hz, 2H), 3.83 (dd, J=8.9, 6.7 Hz, 1H), 3.49 (t, J=2.4 Hz, 1H), 2.97 (dh, J=26.0, 6.5 Hz, 2H), 1.96 (h, J=6.6 Hz, 1H), 1.74-1.50 (m, 2H), 1.39 (m, 11H), 0.84 (dd, J=16.2, 6.7 Hz, 6H). LCMS: MNa+570.5; Rt=0.79 min (2 min acidic method-Method A).

### Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate

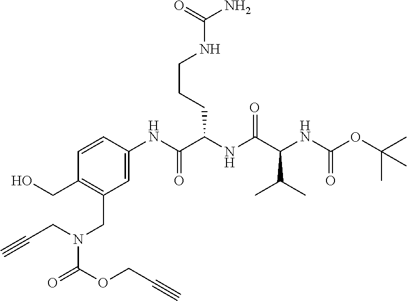 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1CO]

### Step 1: Synthesis of 2-(((tert-butyldiphenylsilyl)oxy)methyl)-5-nitrobenzoic acid

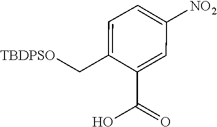

To a solution of 6-nitroisobenzofuran-1(3H)-one (90 g, 502.43 mmol, 1.00 eq) in MeOH (1000 mL) and KOH (28.19 g, 502.43 mmol, 1.00 eq) in H20 (150 mL) was added. The brown mixture was stirred at 25° C. for 1.5h. The brown mixture was concentrated under reduced pressure to give a residue and dissolved in DCM (2000 mL). The mixture was added TBDPSCl (296.91 g, 1.08 mol, 277.49 mL, 2.15 eq) and imidazole (171.03 g, 2.51 mol, 5.00 eq) stirred at 25° C. for 12 h. The mixture was concentrated under reduced pressure to give a residue. The residue was purified by silica gel chromatography (Petroleum ether/Ethyl acetate=I/O, 1/1) and 2-(((tert-butyldiphenylsilyl)oxy)methyl)-5-nitrobenzoic acid (34 g, 74.16 mmol, 14.76% yield) was obtained as a white solid. ¹H NMR (400 MHz, METHANOL-d4) δ ppm 1.13 (s, 9H) 5.26 (s, 2H) 7.34-7.48 (m, 6H) 7.68 (br d, J=8 Hz, 4H) 8.24 (br d, J=8 Hz, 1H) 8.46 (br d, J=8 Hz, 1H) 8.74 (s, 1H)

### Step 2: Synthesis of (2-(((tert-butyldiphenylsilyl)oxy)methyl)-5-nitrophenyl)methanol

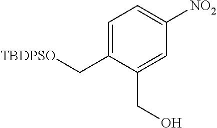

To a mixture of 2-(((tert-butyldiphenylsilyl)oxy)methyl)-5-nitrobenzoic acid (41 g, 94.14 mmol, 1 eq) in THF (205 mL) was added BH₃. THF (1 M, 470.68 mL, 5 eq). The yellow mixture was stirred at 60° C. for 2 h. The mixture was added MeOH (400 mL), and concentrated under reduced pressure to give a residue. then addition of H₂O (200 mL) and DCM (300 mL), extracted with DCM (3×200 mL), washed with brine (300 mL), dried over anhydrous MgSO₄, filtered, and concentrated under reduced pressure to give a residue. The residue was purified by silica gel chromatography (Petroleum ether/Ethyl acetate=I/O, 1/1). (2-(((tert-butyldiphenylsilyl)oxy)methyl)-5-nitrophenyl)methanol (34 g, 80.65 mmol, 85.7% yield) was obtained as a white solid.

### 1H NMR (400 MHz, METHANOL-d4) δ ppm 1.10 (s, 9H) 4.58 (s, 2H) 4.89 (s, 2H) 7.32-7.51 (m, 6H) 7.68 (dd, J=8, 1.38 Hz, 4H) 7.76 (d, J=8 Hz, 1H) 8.15 (dd, J=8 2.26 Hz, 1H) 8.30 (d, J=2 Hz, 1 H)

Step 3: Synthesis of 2-(((tert-butyldiphenylsilyl)oxy)methyl)-5-nitrobenzaldehyde

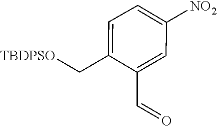

To a solution of (2-(((tert-butyldiphenylsilyl)oxy)methyl)-5-nitrophenyl)methanol (34 g, 80.65 mmol, 1 eq) in DCM (450 mL) was added MnO2 (56.09 g, 645.22 mmol, 8 eq). The black mixture was stirred at 25° C. for 36 h. The mixture was added MeOH(400 mL), and concentrated under reduced pressure to give a residue. then addition of H₂O (200 mL) and DCM (300 mL), extracted with DCM (3×200 mL), washed with brine (300 mL), dried over anhydrous MgSO₄, filtered, and concentrated under reduced pressure to give a residue. The residue was purified by silica gel chromatography (CH₂Cl₂=100%). 2-(((tert-butyldiphenylsilyl)oxy)methyl)-5-nitrobenzaldehyde (30 g, 71.51 mmol, 88.7% yield) was obtained as a white solid.

¹H NMR (400 MHz, CHLOROFORM-d) δ ppm 1.14 (s, 9H) 5.26 (s, 2H) 7.34-7.53 (m, 6H) 7.60-7.73 (m, 4H) 8.13 (d, J=8 Hz, 1H) 8.48 (dd, J=8, 2.51 Hz, 1H) 8.67 (d, J=2 Hz, 1H) 10.16 (s, 1 H)

### Step 4: Synthesis of N-(2-(((tert-butyldiphenylsilyl)oxy)methyl)-5-nitrobenzyl)prop-2-yn-1-amine

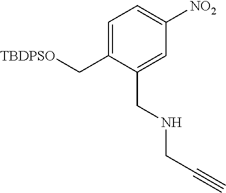

To a solution of 2-(((tert-butyldiphenylsilyl)oxy)methyl)-5-nitrobenzaldehyde (12.6 g, 30.03 mmol, 1 eq) in DCM (130 mL) was added prop-2-yn-1-amine (4.14 g, 75.08 mmol, 4.81 mL, 2.5 eq) and MgSO₄ (36.15 g, 300.33 mmol, 10 eq) then the suspension mixture was stirred at 25° C. for 24 hr. Take a little reaction solution and treat with NaBH₄ the TLC showed one new point was formed. The reaction mixture was filtered and concentrated under reduced pressure to give a residue. (E)-N-[[2-[[tert-butyl(diphenyl)silyl]oxymethyl]-5-nitro-phenyl]methyl]prop-2-yn-1-imine (12 g, crude) was obtained as a yellow solid. ¹H NMR (400 MHz, CHLOROFORM-d) δ ppm 1.11 (s, 9H) 2.48 (t, J=2.38 Hz, 1H) 4.52 (t, J=2.13 Hz, 2H) 5.09 (s, 2H) 7.35-7.49 (m, 6H) 7.63-7.72 (m, 4H) 7.79 (d, J=8.53 Hz, 1H) 8.25 (dd, J=8.53, 2.51 Hz, 1H) 8.68 (d, J=2.26 Hz, 1H) 8.84 (t, J=1.88 Hz, 1H).

(E)-N-[[2-[[tert-butyl(diphenyl)silyl]oxymethyl]-5-nitro-phenyl]methyl]prop-2-yn-1-imine (12 g, 26.28 mmol, 1 eq) was dissolved in MeOH (100 mL) and THF (50 mL), then NaBH₄ (1.49 g, 39.42 mmol, 1.5 eq) was added and the yellow mixture was stirred at −20° C. for 2 hr. LCMS showed that the desired compound was detected. The reaction mixture was quenched by addition MeOH 200 mL at -20° C., and then concentrated under reduced pressure to give a residue. The residue was dissolved with EtOAc 500 mL washed with brine 150 mL, dried over anhydrous Na₂SO₄, filtered and concentrated under reduced pressure to give a residue. The residue was purified by flash silica gel chromatography (Eluent of 0-10% Ethyl acetate/Petroleum ether gradient). N-(2-(((tert-butyldiphenylsilyl)oxy)methyl)-5-nitrobenzyl)prop-2-yn-1-amine (9 g, 18.45 mmol, 70% yield) was obtained as a pale yellow oil. ¹H NMR (400 MHz, CHLOROFORM-d) δ ppm 1.12 (s, 9H) 2.13 (t, J=2.38 Hz, 1H) 3.33 (d, J=2.51 Hz, 2H) 3.80 (s, 2H) 4.93 (s, 2H) 7.36-7.49 (m, 6H) 7.69 (dd, J=7.91, 1.38 Hz, 4H) 7.77 (d, J=8.53 Hz, 1H) 8.16 (dd, J=8.41, 2.38 Hz, 1H) 8.24 (d, J=2.26 Hz, 1H).

### Step 5: Synthesis of (9H-fluoren-9-yl)methyl (2-(((tert-butyldiphenylsilyl)oxy)methyl)-5-nitrobenzyl)(prop-2-yn-1-yl)carbamate

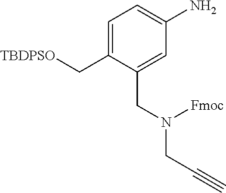

A solution of N-(2-(((tert-butyldiphenylsilyl)oxy)methyl)-5-nitrobenzyl)prop-2-yn-1-amine (9 g, 19.62 mmol, 1 eq) and Fmoc-OSU (7.28 g, 21.59 mmol, 1.1 eq) in dioxane (90 mL) was added sat. NaHCO₃ (90 mL) and the white suspension was stirred at 20° C. for 12 hr. The reaction mixture was diluted with H₂O 150 mL and extracted twice with EtOAc (150 mL each time). The combined organic layers were washed with brine 200 mL, dried over anhydrous Na₂SO₄, filtered and concentrated under reduced pressure to give a residue. The residue was purified by flash silica gel chromatography (Eluent of 0-30% Ethyl acetate/Petroleum ether). (9H-fluoren-9-yl)methyl (2-(((tert-butyldiphenylsilyl)oxy)methyl)-5-nitrobenzyl)(prop-2-yn-1-yl)carbamate (7.7 g, 11.08 mmol, 56.48% yield, 98% purity) was obtained as a white solid.

1H NMR (400 MHz, CHLOROFORM-d) δ ppm 1.12 (s, 9H) 2.17 (br d, J=14.31 Hz, 1H) 3.87-4.97 (m, 9H) 6.98-8.28 (m, 21H).

### Step 6: Synthesis of (9H-fluoren-9-yl)methyl (5-amino-2-(((tert-butyldiphenylsilyl)oxy)methyl)benzyl)(prop-2-yn-1-yl)carbamate

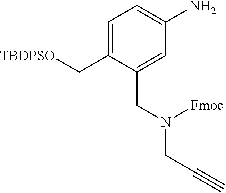

To an ice bath cooled solution of (9H-fluoren-9-yl)methyl (2-(((tert-butyldiphenylsilyl)oxy)methyl)-5-nitrobenzyl)(prop-2-yn-1-yl)carbamate (5.0 g, 7.34 mmol, 1.0 equiv.) in 10% AcOH/CH₂Cl₂ (100 mL) was added Zn (7.20 g, 110 mmol, 15 equiv.). The ice bath was removed and the resulting mixture stirred for 2 hours at which time it was filtered through a pad of celite. The volatiles were removed in vacuo and the residue was dissolved in EtOAc, was washed with NaHCO₃ (sat.), NaCl(sat.), dried over MgSO₄, filtered, concentrated and after ISCO SiO₂ chromatography (0-75% EtOAc/Heptanes) (9H-fluoren-9-yl)methyl (5-amino-2-(((tert-butyldiphenylsilyl)oxy)methyl)benzyl)(prop-2-yn-1-yl)carbamate was obtained (2.99 g, 62%). LCMS: MH+=651.6; Rt=3.77 min (5 min acidic method-Method C).

### Step 7: Synthesis of (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate

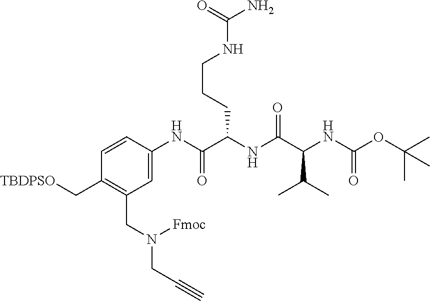

To (9H-fluoren-9-yl)methyl (5-amino-2-(((tert-butyldiphenylsilyl)oxy)methyl)benzyl)(prop-2-yn-1-yl)carbamate (2.99 g, 4.59 mmol, 1.0 equiv) and (S)-2-((S)-2-((tert-butoxycarbonyl)amino)-3-methylbutanamido)-5-ureidopentanoic acid (1.72 g, 4.59 mmol, 1.0 equiv.) in CH₂Cl₂ (40 mL) was added ethyl 2-ethoxyquinoline-1(2H)-carboxylate (2.27 g, 9.18 mmol, 2.0 equiv.). After stirring for 10 minutes, MeOH (1 mL) was added and the solution became homogeneous. The reaction was stirred for 16 hours, the volatiles were removed in vacuo and after purification by ISCO SiO₂ chromatography (0-15% MeOH/CH₂Cl₂) (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate was obtained (2.78 g, 60%). LCMS: MH+=1008.8; Rt=3.77 min (5 min acidic method-Method C).

### Step 8: Synthesis of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate

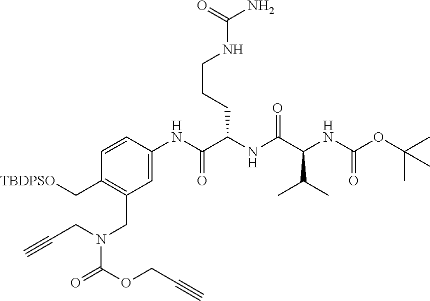

To (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate (1.60 g, 1.588 mmol, 1.0 equiv.) was added 2M dimethylamine in MeOH (30 mL, 60 mmol, 37 equiv.) and THF (10 mL). After standing for 3 hours the volatiles were removed in vacuo and the residue was triturated with Et₂O to remove Fmoc deprotection byproducts. To the resulting solid was added CH₂Cl₂ (16 mL) and pyridine (4 mL) and to the heterogeneous solution was added propargyl chloroformate (155 μL, 1.588 mmol, 1.0 equiv.). After stirring for 30 minutes additional propargyl chloroformate (155 μL, 1.588 mmol, 1.0 equiv.) was added. After stirring for an additional 20 minutes MeOH (1 mL) was added to quench the remaining chloroformate and the volatiles were removed in vacuo. Upon purification by ISCO SiO₂ chromatography (0-15% MeOH/CH₂Cl₂) prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate was obtained (984 mg, 71%). LCMS: MH+=867.8; Rt=3.40 min (5 min acidic method-Method C).

### Step 9: Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate

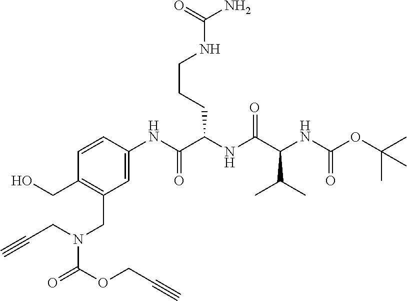 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1CO]

To a solution of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate (984 mg, 1.135 mmol, 1.0 equiv.) in THF (7.5 mL) was added 1.0 M tetrabutylammoniumn fluoride in THF (2.27 mL, 2.27 mmol, 2.0 equiv.). After standing for 6 hours the volatiles were removed in vacuo, the residue was purified by ISCO SiO₂ chromatography (0-40% MeOH/CH₂Cl₂) and prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate was obtained (629 mg, 88%). LCMS: MH+=629.6; Rt=1.74 min (5 min acidic method-Method C).

### Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate

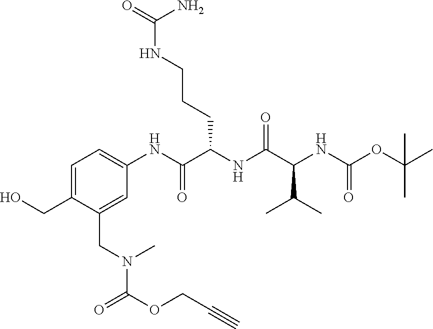 [SMILES: C#CCOC(=O)N(C)Cc1cc(NC(=O)[C@H](CCCNC(N)=O)NC(=O)[C@@H](NC(=O)OC(C)(C)C)C(C)C)ccc1CO]

### Step 1: Synthesis of 2-(hydroxymethyl)-N-methyl-5-nitrobenzamide

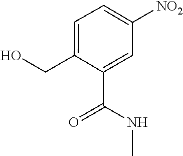

To a stirred suspension of 6-nitroisobenzofuran-1(3H)-one (500 g, 2.79 mol) in MeOH (1500 mL) was added MeNH₂ (3.00 kg, 29.94 mol, 600 mL, 31.0% purity) at 25° C. and stirred for 1 h. The solid was filtered and washed with water twice (600 mL) and dried under high vacuum to get a residue. The product 2-(hydroxymethyl)-N-methyl-5-nitrobenzamide (560 g, crude) was obtained as white solid. LCMS: RT=0.537 min, MS m/z=193.2. 1H NMR: 400 MHz DMSO 6 8.57 (br d, J=4.4 Hz, 1H), 8.31 (dd, J=2.4, 8.6 Hz, 1H), 8.21 (d, J=2.4 Hz, 1H), 7.86 (d, J=8.8 Hz, 1H), 5.54 (t, J=5.6 Hz, 1H), 4.72 (d, J=5.5 Hz, 2H), 2.78 (d, J=4.4 Hz, 3H).

### Step 2: Synthesis of (2-((methylamino)methyl)-4-nitrophenyl)methanol

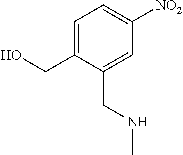

A solution of 2-(hydroxymethyl)-N-methyl-5-nitrobenzamide (560 g, 2.66 mol) in THF (5000 mL) was cooled to 0° C., then BH₃-Me₂S (506 g, 6.66 mol) (2.0 M in THF) was added drop wise for 60 min and the mixture was heated to 70° C. for 5 h. LCMS showed the starting material was consumed. After completion, 4M HCl (1200 mL) in Methanol was added to the reaction mixture at 0° C. and heated at 65° C. for 8 h. The reaction mixture was cooled to 0° C., the solid was filtered and concentrated in reduce pressure. (2-((methylamino)methyl)-4-nitrophenyl)methanol was obtained as a white solid (520 g). LCMS: RT=0.742 min, MS m/z=197.1 [M+H]⁺. ¹H NMR: 400 MHz DMSO 6 9.25 (br s, 2H), 8.37 (d, J=2.4 Hz, 1H), 8.14 (dd, J=2.4, 8.5 Hz, 1H), 7.63 (d, J=8.4 Hz, 1H), 5.72 (br s, 1H), 4.65 (s, 2H), 4.15 (br s, 2H), 2.55-2.45 (m, 3H)

### Step 3: Synthesis of 1-(2-(((tert-butyldiphenylsilyl)oxy)methyl)-5-nitrophenyl)-N-methylmethanamine

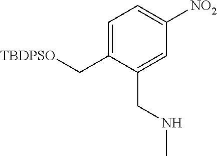

A solution of (2-((methylamino)methyl)-4-nitrophenyl)methanol (520 g, 2.65 mol) and imidazole (721 g, 10.6 mol) in DCM (2600 mL) was cooled to 0° C. then TBDPS-Cl (1.09 kg, 3.98 mol, 1.02 L) was added drop wise and the mixture was stirred for 2 h. The mixture was poured into ice cold water (1000 mL) and extracted with ethyl acetate. The combined organic layers were washed with brine, dried over Na₂SO₄, filtered and evaporated under vacuum to give a crude product. The crude product was purified by chromatography on a silica gel eluted with ethyl acetate:Petroleum ether (from 10/1 to 1) to give a residue. 1-(2-(((tert-butyldiphenylsilyl)oxy)methyl)-5-nitrophenyl)-N-methylmethanamine (600 g) was obtained as a yellow liquid. LCMS: product: RT=0.910 min, MS m/z=435.2 [M+H]⁺

1H NMR: 400 MHz CDCl3 6 8.23 (d, J=2.4 Hz, 1H), 8.15 (dd, J=2.4, 8.4 Hz, 1H), 7.76 (d, J=8.4 Hz, 1H), 7.71-7.66 (m, 4H), 7.50-7.37 (m, 6H), 4.88 (s, 2H), 3.65 (s, 2H), 2.39 (s, 3H), 1.12 (s, 9H)

### Step 4: Synthesis of (9H-fluoren-9-yl)methyl (2-(((tert-butyldiphenylsilyl)oxy)methyl)-5-nitrobenzyl)(methyl)carbamate

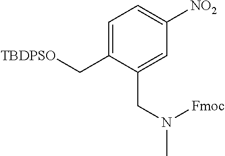

To a solution of 1-(2-(((tert-butyldiphenylsilyl)oxy)methyl)-5-nitrophenyl)-N-methylmethanamine (400 g, 920.3 mmol) in THF (4000 mL) was added Fmoc-OSU (341.5 g, 1.01 mol) and Et₃N (186.2 g, 1.84 mol, 256.2 mL), and the mixture was stirred at 25° C. for 1 h. The mixture was poured into water (1600 mL) and extracted twice with ethyl acetate (1000 mL). The combined organic layers were washed with brine, dried over Na₂SO₄, filtered and evaporated under vacuum to give crude product. The crude product was purified by chromatography on a silica gel eluted with petroleum ether:ethyl acetate (from I/O to 1/1) to give (9H-fluoren-9-yl)methyl (2-(((tert-butyldiphenylsilyl)oxy)methyl)-5-nitrobenzyl)(methyl)carbamate (405 g) as a white solid. LCMS: RT=0.931 min, MS m/z=657.2 [M+H]⁺.

1H NMR: 400 MHz CDCl3 6 8.21-7.96 (m, 1H), 7.87-7.68 (m, 3H), 7.68-7.62 (m, 4H), 7.62-7.47 (m, 2H), 7.47-7.28 (m, 9H), 7.26-7.05 (m, 2H), 4.81 (br s, 1H), 4.62-4.37 (m, 4H), 4.31-4.19 (m, 1H), 4.08-3.95 (m, 1H), 2.87 (br d, J=5.2 Hz, 3H), 1.12 (s, 9H).

### Step 5: Synthesis of (9H-fluoren-9-yl)methyl (5-amino-2-(((tert-butyldiphenylsilyl)oxy)methyl)benzyl)(methyl)carbamate

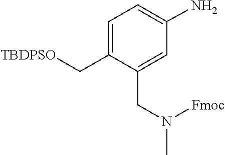

A solution of (9H-fluoren-9-yl)methyl (2-(((tert-butyldiphenylsilyl)oxy)methyl)-5-nitrobenzyl)(methyl)carbamate (3.0 g, 4.57 mmol, 1.0 equiv.) in MeOH (90 mL) and EtOAc (30 mL) was degassed and purged to a balloon of N₂ via three way stopcock. After repeating degas/N₂ purge 2x, 10% Pd/C deGussa type (0.486 g, 0.457 mmol, 0.1 equiv.) was added. The resulting mixture was degassed and purged to a balloon of 2 H₂ via three way stopcock. After repeating degas/H₂ purge 2×, the reaction stirred under the balloon pressure of H₂ for 4 hours. The reaction was degassed and purged to N₂, filtered through a pad of celite eluting further with MeOH. After removal of the volatiles in vacuo and pumping on high vac (9H-fluoren-9-yl)methyl (5-amino-2-(((tert-butyldiphenylsilyl)oxy)methyl)benzyl)(methyl)carbamate was obtained (2.78 g, 97%). LCMS: MH+=627.7; Rt=1.59 min (2 min acidic method-Method A). ¹H NMR: 400 MHz CDCl3 6 7.80 (br d, J=7.2 Hz, 1H), 7.74-7.67 (m, 5H), 7.64 (br d, J=6.8 Hz, 1H), 7.49-7.30 (m, 10H), 7.23-7.06 (m, 2H), 6.61-6.41 (m, 2H), 4.66 (br d, J=7.2 Hz, 2H), 4.55 (s, 2H), 4.51-4.34 (m, 2H), 4.32-4.10 (m, 1H), 3.66 (br s, 2H), 2.96-2.78 (m, 3H), , 1.07 (s, 9H).

### Step 6: Synthesis of (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate

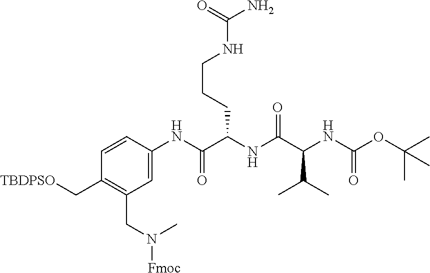

To (9H-fluoren-9-yl)methyl (5-amino-2-(((tert-butyldiphenylsilyl)oxy)methyl)benzyl)(methyl)carbamate (2.86 g, 4.56 mmol, 1.0 equiv) and (S)-2-((S)-2-((tert-butoxycarbonyl)amino)-3-methylbutanamido)-5-ureidopentanoic acid (1.71 g, 4.56 mmol, 1.0 equiv.) in 2:1 CH2Cl2/MeOH (60 mL) was added ethyl 2-ethoxyquinoline-1(2H)-carboxylate (2.256 g, 9.12 mmol, 2.0 equiv.). The homogeneous solution was stirred for 16 hours at which time additional (S)-2-((S)-2-((tert-butoxycarbonyl)amino)-3-methylbutanamido)-5-ureidopentanoic acid (0.340 g, 0.2 equiv.) and ethyl 2-ethoxyquinoline-1(2H)-carboxylate (0.452 g, 0.4 equiv.) were added to drive the reaction to completion. After stirring for an additonal 5 hours the volatiles were removed in vacuo and after purification by ISCO SiO₂ chromatography (0-5% MeOH/CH₂Cl₂) (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate was obtained (2.95 g, 65%). LCMS: MH+=984.1; Rt=1.54 min (2 min acidic method-Method A).

### Step 7: Synthesis of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate

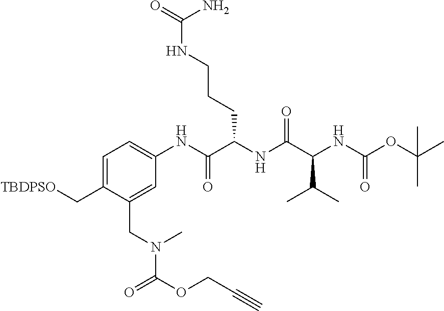

To (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (2.05 g, 2.085 mmol, 1.0 equiv) in THF (10 mL) was added 2.0 M dimethyl amine in MeOH (10.42 mL, 20.85 mmol, 10 equiv.). After stirring for 16 hours the volatiles were removed in vacuo. The residue was dissolved in CH₂Cl₂ (20 mL) and DIEA (0.533 mL, 4.17 mmol, 2 equiv.) and propargyl chloroformate (0.264 mL, 2.71 mmol, 1.3 equiv.) were added. After stirring at rt for 16 hours the reaction was diluted with CH₂Cl₂ (20 mL), was washed with NaHCO₃ (sat.), NaCl(sat.), dried over MgSO₄, filtered, concentrated and purified by ISCO SiO₂ chromatography (0-15% MeOH/CH₂Cl₂) to yield prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (1.04 grams, 59%). LCMS: MH+=843.8; Rt=1.35 min (2 min acidic method-Method A).

### Step 8: Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate

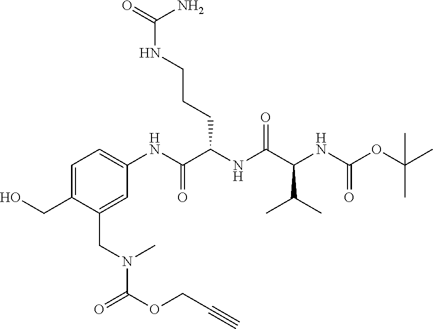 [SMILES: C#CCOC(=O)N(C)Cc1cc(NC(=O)[C@H](CCCNC(N)=O)NC(=O)[C@@H](NC(=O)OC(C)(C)C)C(C)C)ccc1CO]

To a 0° C. solution of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (1.6 g, 1.90 mmol, 1.0 equiv.) in THF (10.0 mL) was added 1.0 M tetrabutylammonium fluoride in THF (3.80 mL, 3.80 mmol, 2.0 equiv.). After warming to rt and stirring for 16 hours the volatiles were removed in vacuo, the residue was dissolved in EtOAc, was washed with NaHCO₃ (sat.), with NaCl(sat.), dried over MgSO₄, filtered, concentrated and the residue was purified by ISCO SiO₂ chromatography (0-30% MeOH/CH₂Cl₂) to yield prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate (1.0 g, 87%). LCMS: MH+=605.7; Rt=0.81 min (2 min acidic method-Method A).

### Synthesis of tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate

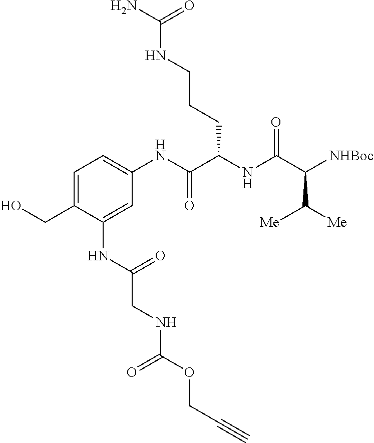 [SMILES: C#CCOC(=O)NCC(=O)Nc1cc(NC(=O)[C@H](CCCNC(N)=O)NC(=O)[C@@H](NC(=O)OC(C)(C)C)C(C)C)ccc1CO]

### Step 1: Synthesis of (9H-fluoren-9-yl)methyl (S)-(1-((4-(hydroxymethyl)-3-nitrophenyl)amino)-1-oxo-5-ureidopentan-2-yl)carbamate

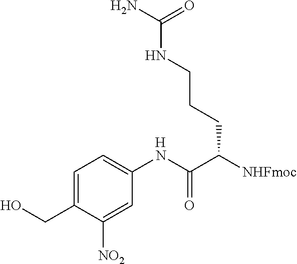

To a solution of (4-amino-2-nitrophenyl)methanol (10 g, 59.5 mmol, 1.0 equiv.), (9H-fluoren-9-yl)methyl (S)-(1-amino-1-oxo-5-ureidopentan-2-yl)carbamate (23.64 g, 59.5 mmol, 1.0 equiv.), and 1-hydroxy-7-azabenzotriazole (8.50 g, 62.4 mmol, 1.05 equiv.) in DMF (50 mL) was added 1-(3-dimethylaminopropyl)-3-ethylcarbodiimide (11.97 g, 62.4 mmol, 1.05 equiv.). After stirring at ambient temperature for 16 hours, the mixture was poured into water (4 L) and stirred for 30 minutes. The resulting solid was filtered, rinsed with water, and dried under vacuum. (9H-Fluoren-9-yl)methyl (S)-(1-((4-(hydroxymethyl)-3-nitrophenyl)amino)-1-oxo-5-ureidopentan-2-yl)carbamate was obtained (31.49 g, 57.5 mmol, 97%). LCMS: MH+=548; Rt=2.02 min (5 min acidic method-Method C).

### Step 2: Synthesis of (S)-2-amino-N-(4-(hydroxymethyl)-3-nitrophenyl)-5-ureidopentanamide

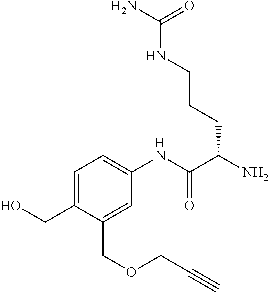

To a solution of (9H-Fluoren-9-yl)methyl (S)-(1-((4-(hydroxymethyl)-3-nitrophenyl)amino)-1-oxo-5-ureidopentan-2-yl)carbamate (31.49 g, 57.5 mmol, 1.0 equiv.) in DMF (50 mL) was added dimethylamine (2 M in MeOH, 331 mL, 661 mmol, 11.5 equiv.). After stirring at ambient temperature for 24 hours, the volatiles were removed under vacuum and the resulting residue was triturated with diethyl ether (3×2 L). The resulting residue was dried under vacuum and (S)-2-amino-N-(4-(hydroxymethyl)-3-nitrophenyl)-5-ureidopentanamide was obtained (21.85 g, 57.5 mmol, 99%). LCMS: MH+=326.4; Rt=0.35 min (2 min acidic method-Method A).

### Step 3: Synthesis of tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate

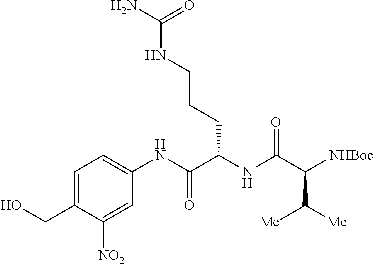 [SMILES: CC(C)[C@H](NC(=O)OC(C)(C)C)C(=O)N[C@@H](CCCNC(N)=O)C(=O)Nc1ccc(CO)c([N+](=O)[O-])c1]

To a solution of (S)-2-amino-N-(4-(hydroxymethyl)-3-nitrophenyl)-5-ureidopentanamide (10.89 g, 28.8 mmol, 1.0 equiv.), (tert-butoxycarbonyl)-L-valine (6.25 g, 28.8 mmol, 1.0 equiv.), and 1-hydroxy-7-azabenzotriazole (3.92 g, 28.8 mmol, 1.0 equiv.) in DMF (40 mL) was added 1-(3-dimethylaminopropyl)-3-ethylcarbodiimide (5.52 g, 28.8 mmol, 1.0 equiv.). After stirring at ambient temperature for 24 hours, the mixture was added dropwise to water (2 L), stirred for 30 minutes, and cooled to 4° C. overnight. The mixture was saturated with NaCl, and the resulting solids were filtered off and dried under vacuum. Tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate was obtained (11.96 g, 22.8 mmol, 79%). LCMS: MH+=525.4; Rt=0.79 min (2 min acidic method-Method A).

### Step 4: Synthesis of tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate

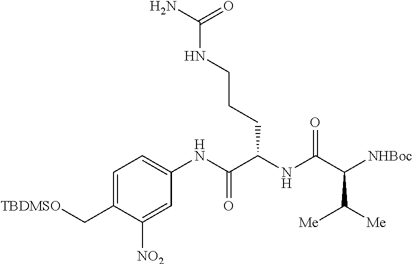 [SMILES: CC(C)[C@H](NC(=O)OC(C)(C)C)C(=O)N[C@@H](CCCNC(N)=O)C(=O)Nc1ccc(CO[Si](C)(C)C(C)(C)C)c([N+](=O)[O-])c1]

To a suspension of tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate (11.96 g, 22.8 mmol, 1.0 equiv.) and imidazole (15.52 g, 228 mmol, 10 equiv.) in DMF (31 mL) was added tert-butyldimethylchlorosilane (13.68 g, 90.76 mmol, 4.0 equiv.). The resulting mixture was stirred at ambient temperature for 48 hours, then heated at 45° C. for 4 hours. The mixture was poured into water and stirred for 96 hours. Solids were filtered and washed with water (2×100 mL) and dried under vacuum. After purification by SiO₂ ISCO chromatography (0-30% methanol/dichloromethane), tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate was obtained (8.02 g, 12.56 mmol, 55%). LCMS: MH+=639.6; Rt=1.22 min (2 min acidic method-Method A).

### Step 5: Synthesis of tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate

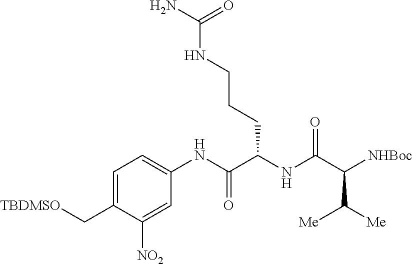 [SMILES: CC(C)[C@H](NC(=O)OC(C)(C)C)C(=O)N[C@@H](CCCNC(N)=O)C(=O)Nc1ccc(CO[Si](C)(C)C(C)(C)C)c([N+](=O)[O-])c1]

To a solution of tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate (8.02 g, 12.56 mmol, 1.0 equiv.) in methanol (250 mL) under a nitrogen atmosphere was added palladium on carbon (10 wt %, 2.00 g, 1.884 mmol, 0.15 equiv.). The mixture was placed under 1 atm dihydrogen and allowed to stir at ambient temperature for 18 hours. The mixture was filtered through celite and dried under vacuum. After purification by SiO₂ ISCO chromatography (0-40% methanol/dichloromethane), tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate was obtained (4.82 g, 7.92 mmol, 63%). LCMS: MH+=609.6; Rt=2.65 min (5 min acidic method-Method C).

### Step 6: Synthesis of tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate

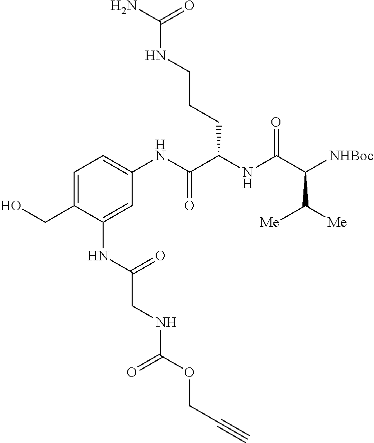 [SMILES: C#CCOC(=O)NCC(=O)Nc1cc(NC(=O)[C@H](CCCNC(N)=O)NC(=O)[C@@H](NC(=O)OC(C)(C)C)C(C)C)ccc1CO]

To a solution of glycine (3.19 g, 42.5 mmol, 1.0 equiv.) in 2 M aqueous sodium hydroxide solution (63.3 mL, 127 mmol NaOH, 3.0 equiv.) was added propargyl chloroformate (5.0 g, 42.5 mmol, 1.0 equiv.). The resulting mixture was stirred at ambient temperature for 3 hours. The mixture was extracted with ethyl acetate (3×250 mL). The combined organic layers were dried over magnesium sulfate, filtered and the volatiles removed under vacuum. After drying, ((prop-2-yn-1-yloxy)carbonyl)glycine,

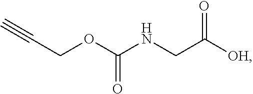

was obtained (3.97 g, 25.3 mmol, 59%). ¹H NMR (400 MHz, DMSO-d₆) δ ppm 3.48 (t, J=2.40 Hz, 1H) 3.66 (d, J=6.19 Hz, 2H) 4.63 (d, J=2.40 Hz, 2H) 7.63 (t, J=6.13 Hz, 1H) 12.57 (br s, 1H).

### Step 6b)

To a solution of tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate (2.7 g, 4.43 mmol, 1.0 equiv.) in DMF (5 mL) were added ((prop-2-yn-1-yloxy)carbonyl)glycine (0.732 g, 4.66 mmol, 1.05 equiv.), 1-hydroxy-7-azabenzotriazole (0.664 g, 4.88 mmol, 1.1 equiv.), and 1-(3-dimethylaminopropyl)-3-ethylcarbodiimide hydrochloride (0.935 g, 4.88 mmol, 1.1 equiv). The resulting mixture was stirred at ambient temperature for 1 hour, then dripped into water (500 mL) and stirred for a further 20 minutes. The resulting precipitate was filtered, washed with water, and dried under vacuum. After purification by SiO₂ ISCO chromatography (0-50% methanol/dichloromethane), tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate was obtained (1.52 g, 2.40 mmol, 54%). LCMS: MH+=634.6; Rt=1.97 min (5 min acidic method-Method C). ¹H NMR (400 MHz, DMSO-d₆) δ ppm 0.76-0.91 (m, 6H) 1.30-1.47 (m, 11H) 1.51-1.73 (m, 2H) 1.87-2.00 (m, 1H) 2.89-3.07 (m, 2H) 3.50 (t, J=2.32 Hz, 1H) 3.73-3.87 (m, 3H) 4.37-4.47 (m, 3H) 4.65 (d, J=2.45 Hz, 2H) 5.30 (t, J=5.44 Hz, 1H) 5.38 (s, 2H) 5.96 (t, J=5.81 Hz, 1H) 6.72 (br d, J=8.93 Hz, 1H) 7.25 (d, J=8.44 Hz, 1H) 7.45 (dd, J=8.25, 2.02 Hz, 1H) 7.78 (br t, J=5.87 Hz, 1H) 7.87-8.00 (m, 2H) 9.51 (s, 1H) 10.04 (s, 1H).

Synthesis of tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate

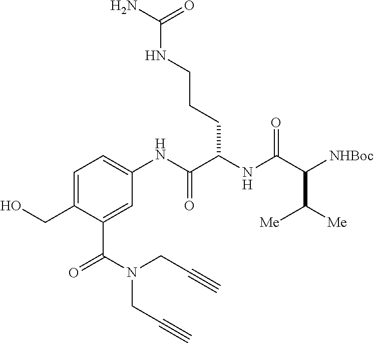 [SMILES: C#CCN(CC#C)C(=O)c1cc(NC(=O)[C@H](CCCNC(N)=O)NC(=O)[C@@H](NC(=O)OC(C)(C)C)C(C)C)ccc1CO]

### Step 1: Synthesis of 2-(((tert-butyldiphenylsilyl)oxy)methyl)-5-nitro-N,N-di(prop-2-yn-1-yl)benzamide

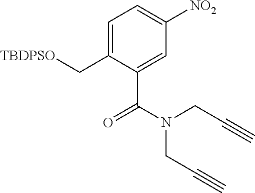

To a solution of 2-(((tert-butyldiphenylsilyl)oxy)methyl)-5-nitrobenzoic acid (1.00 g, 2.30 mmol, 1.0 equiv.) and dipropargylamine (0.257 g, 2.76 mmol, 1.2 equiv.) in dichloromethane (6 mL) were added (1-[bis(dimethylamino)methylene]-1H-1,2,3-triazolo[4,5-b]pyridinium 3-oxid hexafluorophosphate (1.048 g, 2.76 mmol, 1.2 equiv.) and N,N-diisopropylethylamine (0.445 g, 3.44 mmol, 1.5 equiv.). The resulting mixture was stirred at ambient temperature for 1 hour, then diluted with water, extracted with diethyl ether (3×25 mL), dried over sodium sulfate and concentrated. After purification by SiO₂ ISCO chromatography (0-100% ethyl acetate/heptanes), 2-(((tert-butyldiphenylsilyl)oxy)methyl)-5-nitro-N,N-di(prop-2-yn-1-yl)benzamide was obtained (1.08 g, 2.115 mmol, 92%). 1H NMR (400 MHz, Chloroform-d) 6 8.35 (dd, J=8.6, 2.3 Hz, 1H), 8.20 (d, J=2.3 Hz, 1H), 8.02-7.92 (m, 1H), 7.71-7.62 (m, 4H), 7.51 -7.35 (m, 6H), 4.87 (s, 2H), 4.39 (s, 2H), 3.80 (s, 2H), 2.21 (s, 1H), 2.08 (d, J=7.7 Hz, 1H), 1.13 (s, 9H).

### Step 2: Synthesis of 5-amino-2-(((tert-butyldiphenylsilyl)oxy)methyl)-N,N-di(prop-2-yn-1-yl)benzamide

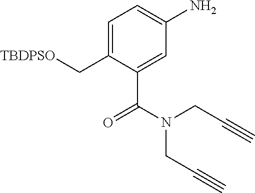

To a stirred suspension of 2-(((tert-butyldiphenylsilyl)oxy)methyl)-5-nitro-N,N-di(prop-2-yn-1-yl)benzamide (1.08 g, 2.115 mmol, 1.0 equiv.) in ethanol (4 mL) and water (4 mL) was added zinc powder (0.553 g, 8.46 mmol, 4 equiv.) and ammonium chloride (0.453 g, 8.46 mmol, 4 equiv.). The resulting mixture was stirred at ambient temperature for 24 hours, then diluted with water and extracted with ethyl acetate (3×25 mL). The combined organic layers were dried over sodium sulfate, filtered and concentrated. After drying under vacuum, 5-amino-2-(((tert-butyldiphenylsilyl)oxy)methyl)-N,N-di(prop-2-yn-1-yl)benzamide was obtained (972 mg, 2.02 mmol, 96%). LCMS: MH+=481.4; Rt=1.33 min (2 min acidic method-Method A).

### Step 3: Synthesis of (S)-5-(2-amino-5-ureidopentanamido)-2-(((tert-butyldiphenylsilyl)oxy)methyl)-N,N-di(prop-2-yn-1-yl)benzamide

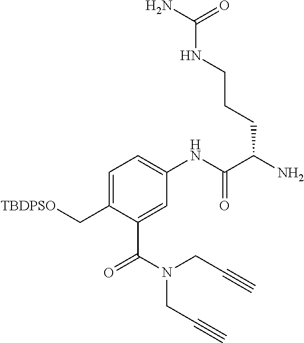

To a solution of 5-amino-2-(((tert-butyldiphenylsilyl)oxy)methyl)-N,N-di(prop-2-yn-1-yl)benzamide (972 mg, 2.02 mmol, 1.0 equiv.), (9H-fluoren-9-yl)methyl (S)-(1-amino-1-oxo-5-ureidopentan-2-yl)carbamate (804 mg, 2.02 mmol, 1.0 equiv.), and (1-[bis(dimethylamino)methylene]-1H-1,2,3-triazolo[4,5-b]pyridinium 3-oxid hexafluorophosphate (846 mg, 2.22 mmol, 1.1 equiv.) in DMF (4 mL) was added N,N-diisopropylethylamine (0.53 mL, 3.03 mmol, 1.5 equiv.). The resulting mixture was stirred at ambient temperature for 18 hours, then poured into water (400 mL) and stirred for 3 hours. The precipitate was filtered and dried under vacuum, then dissolved in a 2 M solution of dimethylamine in tetrahydrofuran (2.02 mL, 4.04 mmol, 2 equiv.) and stirred at ambient temperature for 4 hours. The volatiles were removed under vacuum and after purification by SiO₂ ISCO chromatography, (S)-5-(2-amino-5-ureidopentanamido)-2-(((tert-butyldiphenylsilyl)oxy)methyl)-N,N-di(prop-2-yn-1-yl)benzamide was obtained (1.018 g, 1.596 mmol, 79%). LCMS: MH+=638.6; Rt=1.22 min (2 min acidic method-Method A).

### Step 4: Synthesis of tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate

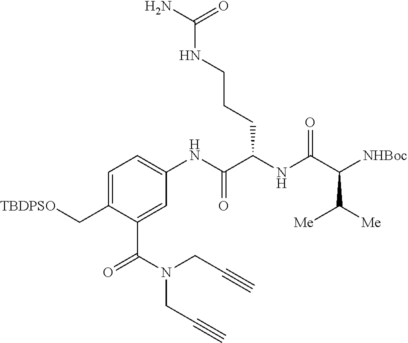

To a solution of (S)-5-(2-amino-5-ureidopentanamido)-2-(((tert-butyldiphenylsilyl)oxy)methyl)-N,N-di(prop-2-yn-1-yl)benzamide (1.00 g, 1.568 mmol, 1.0 equiv.), (tert-butoxycarbonyl)-L-valine (0.341 g, 1.568 mmol, 1.0 equiv.), and (1-[bis(dimethylamino)methylene]-1H-1,2,3-triazolo[4,5-b]pyridinium 3-oxid hexafluorophosphate (0.656 g, 1.725 mmol, 1.1 equiv.) in DMF (3 mL) was added N,N-diisopropylethylamine (0.41 mL, 2.352 mmol, 1.5 equiv). After stirring at ambient temperature for 1 hour, the mixture was diluted with water (30 mL) and brine (30 mL) and extracted with ethyl acetate (3×50 mL). The combined organic layers were dried over sodium sulfate, filtered and concentrated. After purification of the resulting residue by SiO₂ ISCO chromatography (0-50% methanol/dichloromethane), tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate was obtained (1.30 g, 1.553 mmol, 99%). LCMS: MH+=837.5; Rt=1.32 min (2 min acidic method-Method A).

### Step 5: Synthesis of tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate

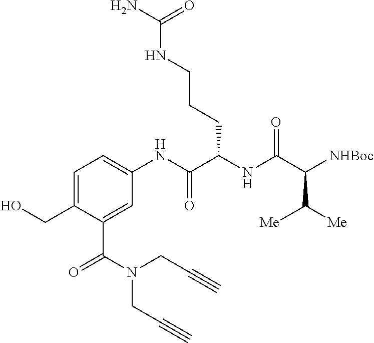 [SMILES: C#CCN(CC#C)C(=O)c1cc(NC(=O)[C@H](CCCNC(N)=O)NC(=O)[C@@H](NC(=O)OC(C)(C)C)C(C)C)ccc1CO]

To a stirred solution of tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate (1.30 g, 1.553 mmol, 1.0 equiv.) in tetrahydrofuran (5 mL), a 1 M solution of tetrabutylammonium fluoride in tetrahydrofuran (3.11 mL, 3.11 mmol, 2.0 equiv.) was added dropwise. After stirring at ambient temperature for 18 hours, the solvent was removed under vacuum. After purification by SiO₂ ISCO chromatography (0-50% methanol/dichloromethane), tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate was obtained (0.703 g, 1.174 mmol, 76%). LCMS: MH+=599.4; Rt=0.76 min (2 min acidic method-Method A). 1H NMR (400 MHz, Methanol-d4) δ 7.71-7.59 (m, 2H), 7.52-7.43 (m, 1H), 4.51 (d, J=29.4 Hz, 4H), 4.11-4.04 (m, 2H), 3.95-3.85 (m, 1H), 3.28-3.06 (m, 2H), 2.76 (m, 2H), 2.11-2.03 (m, 1H), 1.97-1.83 (m, 1H), 1.75 (dtd, J=14.2, 9.4, 5.1 Hz, 1H), 1.70-1.51 (m, 3H), 1.44 (m, 9H), 1.00-0.90 (m, 6H).

### Synthesis of (9H-fluoren-9-yl)methyl (S)-(5-(2-((tert-butoxycarbonyl)amino)-5-ureidopentanamido)-2-(((tert-butyldiphenylsilyl)oxy)methyl)benzyl)(methyl)carbamate

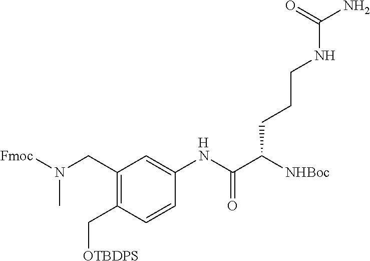

A solution of (S)-2-((tert-Butoxycarbonyl)amino)-5-ureidopentanoic acid (17.57 grams, 63.8 mmol), (9H-fluoren-9-yl)methyl (5-amino-2-(((tert-butyldiphenylsilyl)oxy)methyl)benzyl)(methyl)carbamate (40 grams, 63.8 mmol) and HOAT (1.0 M in DMA, 63.8 mL, 63.8 mmol) in DMF (36 mL) was stirred until homogeneous, cooled in a 0° C. bath and EDC (12.23 grams, 63.8 mmol) was added. The reaction was left stirring as the bath was allowed to warm to rt overnight. After 16 hours, the reaction solution was dripped into 4L H₂O with stirring over ca. 30 minutes at which time the ppt was filtered, rinsed with H₂O (1L) and air dried under vacuum. The wet cake was dissolved in 15% iPrOH/EtOAc (ca. 1.8 L) and washed with H₂O (250 mL), with NaHCO₃ (sat.) (250 mL) and with NaCl(sat.) (250 mL), dried over MgSO₄, filtered, concentrated, and pumped on to yield (9H-fluoren-9-yl)methyl (S)-(5-(2-((tert-butoxycarbonyl)amino)-5-ureidopentanamido)-2-(((tert-butyldiphenylsilyl)oxy)methyl)benzyl)(methyl)carbamate (54.47 grams, 96% yield). LC/MS: MH+=884.7, Rt=3.82 min (5 min. acidic method).

### Synthesis of (9H-fluoren-9-yl)methyl (S)-(5-(2-((tert-butoxycarbonyl)amino)-5-ureidopentanamido)-2-(hydroxymethyl)benzyl)(methyl)carbamate

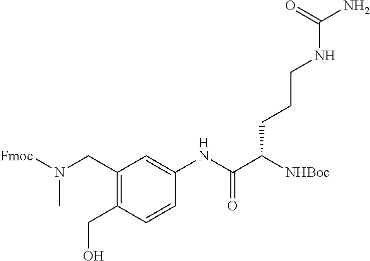

To a solution of (9H-fluoren-9-yl)methyl (S)-(5-(2-((tert-butoxycarbonyl)amino)-5-ureidopentanamido)-2-(((tert-butyldiphenylsilyl)oxy)methyl)benzyl)(methyl)carbamate (33.29 grams, 37.7 mmol) in THF (45 mL) was added AcOH (12.93 mL, 226 mmol) followed by 1.0 M TBAF in THF (56.6 mL, 56.6 mmol). After stirring for 72 hours, the volatiles were removed in vacuo and the residue was partitioned between 15% iPrOH/EtOAc (1.5 L) and H₂O (300 mL), mixed, separated, washed further with H₂O (5×350 mL), with NaHCO₃ (sat.) (2×300 mL), with NaCl(sat.) (300 mL), dried over MgSO₄, filtered, concentrated, pumped on, triturated with Et₂O and filtered to yield (9H-fluoren-9-yl)methyl (S)-(5-(2-((tert-butoxycarbonyl)amino)-5-ureidopentanamido)-2-(hydroxymethyl)benzyl)(methyl)carbamate (19.17 grams, 79% yield). LC/MS: MH+=646.7, Rf=2.28 min. (5 min acidic method).

### Synthesis of (9H-fluoren-9-yl)methyl (S)-(5-(2-amino-5-ureidopentanamido)-2-(hydroxymethyl)benzyl)(methyl)carbamate

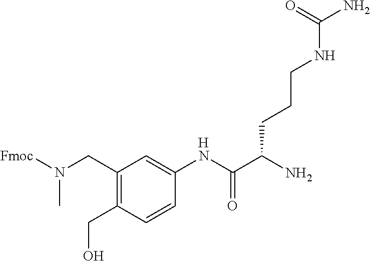

(9H-fluoren-9-yl)methyl (S)-(5-(2-((tert-butoxycarbonyl)amino)-5-ureidopentanamido)-2-(hydroxymethyl)benzyl)(methyl)carbamate (15 g, 23.23 mmol) was dissolved in DCM (30 mL), cooled in ice bath and TFA (28.5 mL, 372 mmol) was added slowly over 5 min. The reaction was kept stirring in the ice bath for 1 hr. The reaction was slowly warmed up to RT and stirred at RT for 1 hour. The volatiles were removed in-vacuo. The product was retaken in dicholoroethane (210 mL). The resulting solution was removed in-vacuo. The process was repeated 2 more times (3 times in total) to obtain a dark yellow solid. The solid was suspended in diethyl ether (300 mL). It was sonicated for 10 min then decanted. The process was repeated 2 more times. The solid was collected by vacuum filtration. After air drying overnight under vacuum, the solid was dissolved in MeOH (100 mL) and this solution was added dropwise over ca. 20 minutes to 7N ammonia in MeOH (201 ml, 402 mmol). After stirring for 30 minutes, the volatiles were removed in-vacuo and, after trituration with Et₂O, (9H-fluoren-9-yl)methyl (S)-(5-(2-amino-5-ureidopentanamido)-2-(hydroxymethyl)benzyl)(methyl)carbamate (15.75 grams) was obtained. LC/MS: MH+=546.5, Rt=1.43 min. (5 min acidic method). The material was used as is for next step.

### Synthesis of (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate

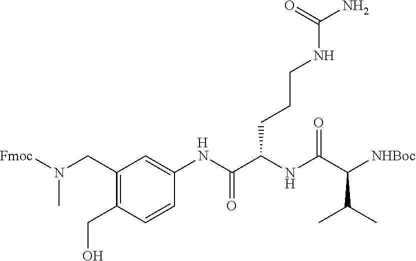

Boc-Val-OH (6.47 g, 29.8 mmol), EDC.HCl (5.48 g, 28.7 mmol) and HOAT (3.89 g, 28.7 mmol) were dissolved in DMF (35 mL) and stirred at RT for 10 min. at which time (9H-fluoren-9-yl)methyl (S)-(5-(2-amino-5-ureidopentanamido)-2-(hydroxymethyl)benzyl)(methyl)carbamate (13 g, 23.83 mmol) was added. DIPEA (4.15 mL, 23.83 mmol) was added. After stirring for 30 minutes the reaction mixture was added dropwise to 4L μof water under vigorous stirring. After 30 minutes, the precipitate was collected by vacuum filtration. The cake was washed with water (1200 mL). The product was air dried under vacuum, was triturated with MTBE, and dried under vacuum for to yield (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (12.8 g, 72% yield). LC/MS: MH+=745.7, 2.37 min. (5 min acidic method).

### Synthesis of (9H-fluoren-9-yl)methyl (5-((S)-2-((S)-2-(((allyloxy)carbonyl)amino)-3-methylbutanamido)-5-ureidopentanamido)-2-(hydroxymethyl)benzyl)(methyl)carbamate

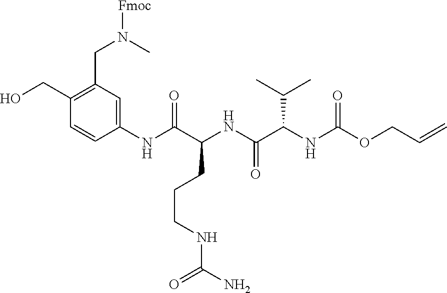

To a solution of (9H-fluoren-9-yl)methyl (S)-(5-(2-amino-5-ureidopentanamido)-2-(hydroxymethyl)benzyl)(methyl)carbamate (9.4 g, 14.25 mmol), HOAT (2.32 g, 17.1 mmol) and ((allyloxy)carbonyl)-L-valine (3.44 g, 17.10 mmol) in DMF (27 mL) was added DIEA (5.53 g, 42.7 mmol) and EDC (3.28 g, 17.1 mmol). After stirring for 2 hours, the solution was dripped into 3L μof H₂O with stirring. The solid formed was collected by vacuum filtration, rinsed further with H₂O and air dried under vacuum to yield (9H-fluoren-9-yl)methyl (5-((S)-2-((S)-2-(((allyloxy)carbonyl)amino)-3-methylbutanamido)-5-ureidopentanamido)-2-(hydroxymethyl)benzyl)(methyl)carbamate (7.69 g, 74% yield). LC/MS: MH+=729.6, Rt=2.27 min (5 min acidic method).

### Synthesis of (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate

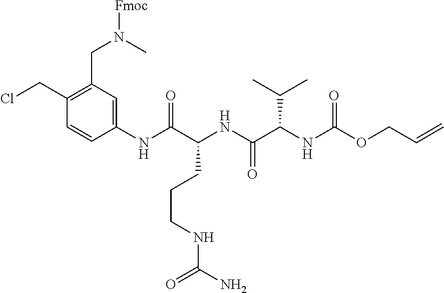

To a heterogeneous solution of (9H-fluoren-9-yl)methyl (5-((S)-2-((S)-2-(((allyloxy)carbonyl)amino)-3-methylbutanamido)-5-ureidopentanamido)-2-(hydroxymethyl)benzyl)(methyl)carbamate (4.0 g, 5.5 mmol) and NaHCO₃ (2.8 g, 33 mmol) in THF (55 mL) at rt was added thionyl chloride (1.0 mL, 14 mmol). After stirring for 1 hour, the solution was partitioned between EtOAc and NaHCO₃ (sat.), extracted further with EtOAc, dried over MgSO₄, filtered, concentrated, purified by SiO₂ chromatography (0-20% iPrOH/CH₂Cl₂eluant). After concentration, (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate (2.1 g, 51% yield) was obtained. LC/MS: MH+=747.6, Rt=2.72 min (5 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate

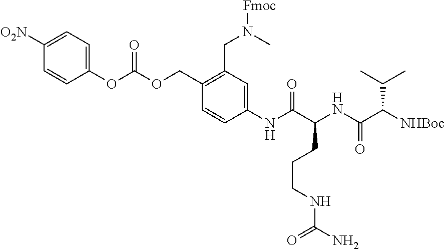

To a solution of (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (2.0 g, 2.685 mmol) and bis(4-nitrophenyl) carbonate (1.634 g, 5.37 mmol) in NMP (7 mL) was added DIEA (1.4 mL, 8.06 mmol). After stirring for 2 hours, the solution was diluted with DMSO and purified by RP-HPLC. After lyophilization, (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (2.0 g, 82% yield) was obtained. HRMS: MH+=910.4000, Rt=3.07 min (5 min acidic method).

### Synthesis of (9H-fluoren-9-yl)methyl (5-((S)-2-((S)-2-amino-3-methylbutanamido)-5-ureidopentanamido)-2-(hydroxymethyl)benzyl)(methyl)carbamate

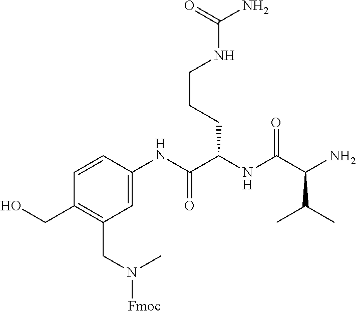

(9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (2.0 grams, 2.68 mmol) was treated with 25% TFA/CH₂Cl₂ (12 mL) in an ice bath. After stirring for one hour, the volatiles were removed in vacuo, dichloroethane was added and volatiles were removed in vacuo. The residue was dissolved in MeOH (20 mL) and the solution was added dropwise to 7M ammonia in MeOH (7.67 mL, 53.7 mmol). After stirring for 30 minutes, the volatiles were removed in vacuo, the residue was dissolved in MeOH and was purified by ISCO RP-HPLC (with no acidic modifier). Upon lyophilization, (9H-fluoren-9-yl)methyl (5-((S)-2-((S)-2-amino-3-methylbutanamido)-5-ureidopentanamido)-2-(hydroxymethyl)benzyl)(methyl)carbamate (1.162 g, 67% yield) was obtained. LC/MS: MH+=645.5, Rt=0.75 min (2 min acidic method).

### Synthesis of (9H-fluoren-9-yl)methyl (5-((S)-2-((S)-2-(2-azidoacetamido)-3-methylbutanamido)-5-ureidopentanamido)-2-(hydroxymethyl)benzyl)(methyl)carbamate

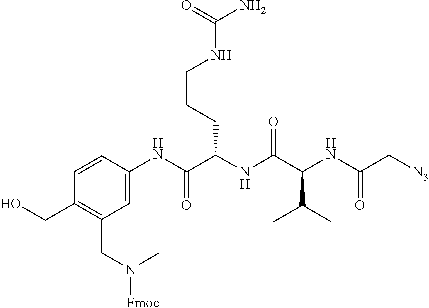

To a solution of (9H-fluoren-9-yl)methyl (5-((S)-2-((S)-2-amino-3-methylbutanamido)-5-ureidopentanamido)-2-(hydroxymethyl)benzyl)(methyl)carbamate (400 mg, 0.620 mmol), 2-azidoacetic acid (60.4 μl, 0.806 mmol) and HATU (307 mg, 0.806 mmol) in DMF(5 ml) was added DIPEA (433 μl, 2.481 mmol). After stirring for 30 min, the solution was diluted with DMSO and purified by ISCO RP-HPLC. Upon lyophilization, (9H-fluoren-9-yl)methyl (5-((S)-2-((S)-2-(2-azidoacetamido)-3-methylbutanamido)-5-ureidopentanamido)-2-(hydroxymethyl)benzyl)(methyl)carbamate (220 mg, 49% yield was obtained. LC/MS: MH+=728.4, Rt=0.96 min (2 min acidic method).

### Synthesis of (9H-fluoren-9-yl)methyl (5-((S)-2-((S)-2-(2-azidoacetamido)-3-methylbutanamido)-5-ureidopentanamido)-2-(chloromethyl)benzyl)(methyl)carbamate

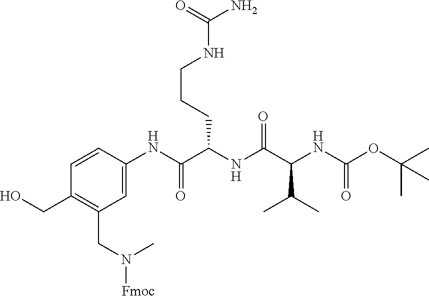

To a slurry of (9H-fluoren-9-yl)methyl (5-((S)-2-((S)-2-(2-azidoacetamido)-3-methylbutanamido)-5-ureidopentanamido)-2-(hydroxymethyl)benzyl)(methyl)carbamate (220 mg, 302 μmol) in CH₂Cl₂(15 mL) at 0° C. was added thionyl chloride (110 p L, 1510 μmol). After stirring for 2 hours, the volatiles were removed in vacuo and the residue was triturated with Et2O. The material was used as is for the next step. LC/MS: MH+=746.4, Rt=1.13 min (2 min acidic method).

### Synthesis of (9H-fluoren-9-yl)methyl (5-((S)-2-((S)-2-(((2-azidoethoxy)carbonyl)amino)-3-methylbutanamido)-5-ureidopentanamido)-2-(hydroxymethyl)benzyl)(methyl)carbamate

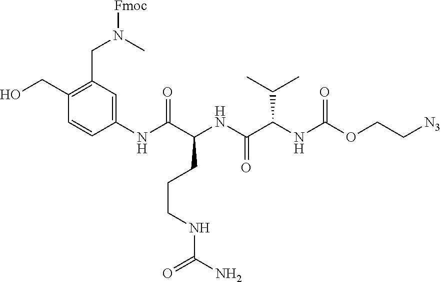

To a solution of (9H-fluoren-9-yl)methyl (5-((S)-2-((S)-2-amino-3-methylbutanamido)-5-ureidopentanamido)-2-(hydroxymethyl)benzyl)(methyl)carbamate TFA salt (1.65 g, 1.89 mmol) and 2-azidoethyl (4-nitrophenyl) carbonate (1.07 g, 4.25 mmol) in DMF (9.5 mL) was added DIEA (986 μL, 5.66 mmol). After stirring for 2 hours, the solution was partitioned between EtOAc and NH4Cl(sat.), washed with brine, dried over MgSO₄, filtered, concentrated, and purified by SiO₂ chromatography (0-30% MeOH/CH₂Cl₂ eluant). After concentration, (9H-fluoren-9-yl)methyl (5-((S)-2-((S)-2-(((2-azidoethoxy)carbonyl)amino)-3-methylbutanamido)-5-ureidopentanamido)-2-(hydroxymethyl)benzyl)(methyl)carbamate (735 mg, 51% yield) was obtained. LC/MS: MH+=758.7, Rt=2.20 min (5 min acidic method).

### Synthesis of (9H-fluoren-9-yl)methyl (5-((S)-2-((S)-2-(((2-azidoethoxy)carbonyl)amino)-3-methylbutanamido)-5-ureidopentanamido)-2-(chloromethyl)benzyl)(methyl)carbamate

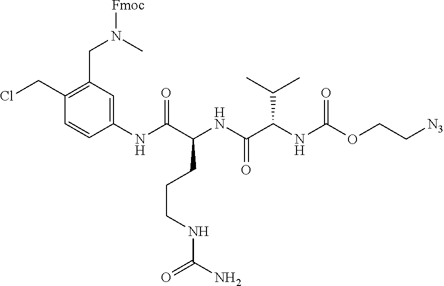

To a solution of (9H-fluoren-9-yl)methyl (5-((S)-2-((S)-2-(((2-azidoethoxy)carbonyl)amino)-3-methylbutanamido)-5-ureidopentanamido)-2-(hydroxymethyl)benzyl)(methyl)carbamate (735 mg, 970 μmol) and NaHCO₃ (570 mg, 6790 μmol) in THF (10 mL) at 0° C. was added thionyl chloride (283 p L, 3880 μmol). After stirring for 1 hour, the solution was partitioned between EtOAc and NaHCO₃ (sat.), extracted further with EtOAc, dried over MgSO₄, filtered, concentrated, purified by SiO₂ chromatography (0−1=20% iPrOH/CH₂Cl₂ eluant). After concentration, (9H-fluoren-9-yl)methyl (5-((S)-2-((S)-2-(((2-azidoethoxy)carbonyl)amino)-3-methylbutanamido)-5-ureidopentanamido)-2-(chloromethyl)benzyl)(methyl)carbamate (578 mg, 77% yield) was obtained. LC/MS: MH+=776.6, Rt=2.66 min (5 min acidic method).

### Synthesis of (9H-fluoren-9-yl)methyl (S)-(5-(2-aminopropanamido)-2-(hydroxymethyl)benzyl)(methyl)carbamate

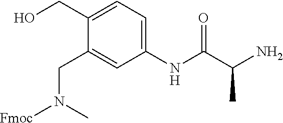

Boc-Ala-OH (0.724 g, 3.83 mmol), EDC.HCl (0.734 g, 3.83 mmol) and HOAT (0.521 g, 3.83 mmol) were dissolved in DMF (3 mL). The reaction was stirred at RT for 10 min at which time (9H-fluoren-9-yl)methyl (5-amino-2-(((tert-butyldiphenylsilyl)oxy)methyl)benzyl)(methyl)carbamate (2 g, 3.19 mmol) was added. The reaction was stirred at RT for 10 min. The reaction mixture was added dropwise to roughly 250 mL of water under vigorous stirring. A precipitate was formed but it was very sticky. The solution was stirred at RT for 30 min and sonicated for 5 min. The precipitate was collected by vacuum filtration. The cake was washed with water (100 mL). The product was air dried under vacuum to obtain the desired amide product (2.36 grams, 93% yield). The amide product was treated with 25% TFA/CH₂Cl₂ (5.33 mL) for one hour at which time the volatiles were removed in vacuo. To clip the TFA ester that has partially formed, the residue was dissolved in MeOH (10 mL) and dripped dropwise into 7N ammonia in MeOH (15.32 mL, 107 mmol). The volatiles were removed in vacuo, the residue was triturated with Et₂O and then pumped on to yield (9H-fluoren-9-yl)methyl (S)-(5-(2-aminopropanamido)-2-(hydroxymethyl)benzyl)(methyl)carbamate (3.08 grams, 94% yield). LC/MS: MH+=460.5, Rt=1.49 min (5 min acidic method). The material was used as is for the next step.

### Synthesis of (9H-fluoren-9-yl)methyl (5-((S)-2-((S)-2-amino-3-methylbutanamido)propanamido)-2-(hydroxymethyl)benzyl)(methyl)carbamate

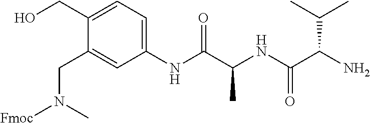

Boc-Val-OH (0.567 g, 2.176 mmol), EDC.HCl (0.501 g, 2.61 mmol) and HOAT (0.355 g, 2.61 mmol) were dissolved in DMF (4 mL). The reaction was stirred at RT for 10 min at which time (9H-fluoren-9-yl)methyl (S)-(5-(2-aminopropanamido)-2-(hydroxymethyl)benzyl)(methyl)carbamate (1 g, 2.176 mmol) was added. The reaction was stirred at RT for 10 min. The reaction mixture was added dropwise to roughly 1 L μof water under vigorous stirring. The solution was stirred at RT for 30 min and sonicated for 5 min. The precipitate was collected by vacuum filtration. The cake was washed with water (100 mL). The product was air dried under vacuum to obtain the desired crude amide product (1.6 grams). The amide product (1.06 g, 1.61 mmol) was treated with 25% TFA/CH₂Cl₂ (5.33 mL) for one hour at which time the volatiles were removed in vacuo. To clip the TFA ester that has partially formed, the residue was dissolved in MeOH (10 mL) and dripped dropwise into 7N ammonia in MeOH (15.32 mL, 107 mmol). The volatiles were removed in vacuo, the residue was dissolved in DMSO and purified by RP-HPLC. Upon lyophilization, (9H-fluoren-9-yl)methyl (5-((S)-2-((S)-2-amino-3-methylbutanamido)propanamido)-2-(hydroxymethyl)benzyl)(methyl)carbamate trifluoroacetate (300 mg, 33% yield) was obtained. LC/MS: MH+=559.6, Rt=1.58 min (5 min acidic method).

### Synthesis of (9H-fluoren-9-yl)methyl (5-((S)-2-((S)-2-(((2-azidoethoxy)carbonyl)amino)-3-methylbutanamido)propanamido)-2-(hydroxymethyl)benzyl)(methyl)carbamate

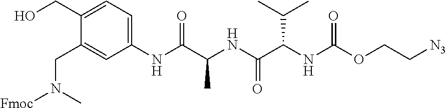

To a solution of (9H-fluoren-9-yl)methyl (5-((S)-2-((S)-2-amino-3-methylbutanamido)propanamido)-2-(hydroxymethyl)benzyl)(methyl)carbamate trifluoroacetate (150 mg, 223 μmol) and 2-azidoethyl (4-nitrophenyl) carbonate (112 mg, 446 μmol) in DMF (1 mL) was added DIEA 1194 μL, 1115 μmol). After stirring for 1 hour, the solution was diluted with DMSO and purified by RP-HPLC. Upon lyophilization, (9H-fluoren-9-yl)methyl (5-((S)-2-((S)-2-(((2-azidoethoxy)carbonyl)amino)-3-methylbutanamido)propanamido)-2-(hydroxymethyl)benzyl)(methyl)carbamate (185 mg, ca. 100% yield) was obtained. LC/MS: (M-H₂O)+H⁺=654.6, Rt=2.45 (5 min acidic method).

### Synthesis of (9H-fluoren-9-yl)methyl (5-((S)-2-((S)-2-(((2-azidoethoxy)carbonyl)amino)-3-methylbutanamido)propanamido)-2-(chloromethyl)benzyl)(methyl)carbamate

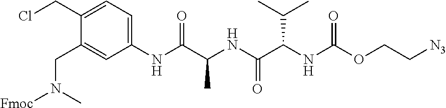

To a mixture of (9H-fluoren-9-yl)methyl (5-((S)-2-((S)-2-(((2-azidoethoxy)carbonyl)amino)-3-methylbutanamido)propanamido)-2-(hydroxymethyl)benzyl)(methyl)carbamate (185 mg, 235 μmol) and sodium bicarbonate (138 mg, 1648 μmol) in THF (7 mL) in an ice bath was added thionyl chloride (69 μL, 942 μmol). After stirring for 30 minutes, the solution was added dropwise into NaHCO₃ (sat.) (100 mL) and was then extracted with EtOAc (3×50 mL). The combined organics were washed with NaCl(sat.), dried over MgSO₄, filtered, concentrated, and purified by SiO₂ chromatography (0-20% iPrOH/CH₂Cl₂ eluant) to yield (9H-fluoren-9-yl)methyl (5-((S)-2-((S)-2-(((2-azidoethoxy)carbonyl)amino)-3-methylbutanamido)propanamido)-2-(chloromethyl)benzyl)(methyl)carbamate (115 mg, 71% yield). LC/MS: MH+=690.6, Rt=2.96 min (5 min acidic method).

### Synthesis of tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxopropan-2-yl)amino)-3-methyl-1-oxobutan-2-yl)amino)-6-oxohexane-1,5-diyl)dicarbamate

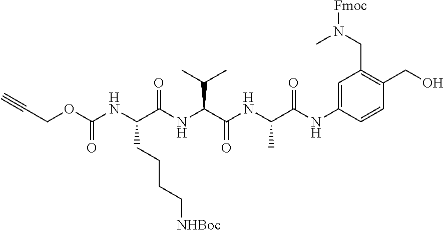

To a solution of N6-(tert-butoxycarbonyl)-N2-((prop-2-yn-1-yloxy)carbonyl)-L-lysine (88 mg, 268 μmol), HOAT (36.4 mg, 268 μmol) and EDC (51.3 mg, 268 μmol) in DMF (2 mL) was stirred for 10 minutes at which time DIEA (117 μL, 669 μmol) and (9H-fluoren-9-yl)methyl (5-((S)-2-((S)-2-amino-3-methylbutanamido)propanamido)-2-(hydroxymethyl)benzyl)(methyl)carbamate TFA salt (150 mg, 223 μmol) were added. After stirring for 20 minutes, the solution was diluted with DMSO and purified by RP-HPLC. Upon lyophilization, tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxopropan-2-yl)amino)-3-methyl-1-oxobutan-2-yl)amino)-6-oxohexane-1,5-diyl)dicarbamate (162 mg, 74% yield) was obtained. LC/MS: MH⁺=869.8, Rt=2.66 min (5 min acidic method).

### Synthesis of tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxopropan-2-yl)amino)-3-methyl-1-oxobutan-2-yl)amino)-6-oxohexane-1,5-diyl)dicarbamate

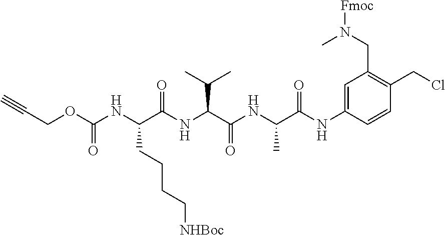

To a mixture of tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxopropan-2-yl)amino)-3-methyl-1-oxobutan-2-yl)amino)-6-oxohexane-1,5-diyl)dicarbamate (162 mg, 165 μmol) and sodium bicarbonate (97 mg, 1154 μmol) in THF (7 mL) in an ice bath was added thionyl chloride (48 μL, 659 μmol). After stirring for 30 minutes, the solution was added dropwise into NaHCO₃ (sat.) (100 mL) and was then extracted with EtOAc (3×50 mL). The combined organics were washed with NaCl(sat.), dried over MgSO₄, filtered, concentrated and purified by SiO₂ chromatography (0-20% iPrOH/CH₂Cl₂ eluant) to yield tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxopropan-2-yl)amino)-3-methyl-1-oxobutan-2-yl)amino)-6-oxohexane-1,5-diyl)dicarbamate (115 mg, 71% yield). LC/MS: MH⁺=887.8, Rt=3.10 min (5 min acidic method).

### Synthesis of tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxo-5-ureidopentan-2-yl)amino)-3-methyl-1-oxobutan-2-yl)amino)-6-oxohexane-1,5-diyl)dicarbamate

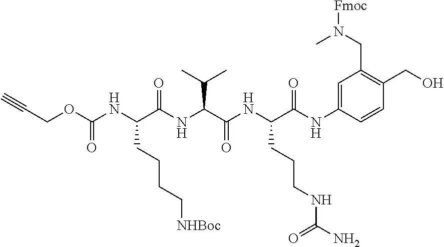

To a solution of N6-(tert-butoxycarbonyl)-N2-((prop-2-yn-1-yloxy)carbonyl)-L-lysine (1.787 g, 5.44 mmol) and HATU (1.88 g, 4.95 mmol) in DMF (20 mL) was added DIEA (862 μL, 4.95 mmol). After stirring for 5 minutes, (9H-fluoren-9-yl)methyl (5-((S)-2-((S)-2-amino-3-methylbutanamido)-5-ureidopentanamido)-2-(hydroxymethyl)benzyl)(methyl)carbamate (3.19 g, 4.95 mmol) in DMF (10 mL) was added. After stirring for an additional 2 hours, the solution dripped into 1L μof H₂O. The resulting solid was collected by filtration, dissolved in CH₂Cl₂/MeOH and purified by SiO₂ chromatography to yield tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxo-5-ureidopentan-2-yl)amino)-3-methyl-1-oxobutan-2-yl)amino)-6-oxohexane-1,5-diyl)dicarbamate (900 mg, 19% yield). LC/MS: MH⁺=955.9, Rt=2.55 min (5 min acidic method).

### Synthesis of tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxo-5-ureidopentan-2-yl)amino)-3-methyl-1-oxobutan-2-yl)amino)-6-oxohexane-1,5-diyl)dicarbamate

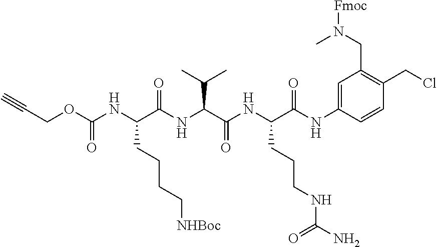

To a mixture of tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxo-5-ureidopentan-2-yl)amino)-3-methyl-1-oxobutan-2-yl)amino)-6-oxohexane-1,5-diyl)dicarbamate (507 mg, 531 μmol) in MeCN (8 mL) in an ice bath was added thionyl chloride (97 μL, 1327 μmol). After stirring for 60 minutes, the volatiles were removed in vacuo, the residue was dissolved in CH₂Cl₂ (20 mL)+MeOH (1 mL) and Boc2O (246 mg, 1062 μmol) and DIEA (556 μL, 3180 μmol) were added. After stirring for 60 minutes, the volatiles were removed in vacuo, the residue was dissolved in CH₂Cl₂ and was purified by SiO₂ chromatography (0-30% MeOH/CH₂Cl₂) to yield tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxo-5-ureidopentan-2-yl)amino)-3-methyl-1-oxobutan-2-yl)amino)-6-oxohexane-1,5-diyl)dicarbamate (342 mg, 66% yield). LC/MS: MH⁺=973.9, Rt=1.21 min (2 min acidic method).

### Synthesis of N-(4-((S)-2-((S)-2-((tert-butoxycarbonyl)amino)-3-methylbutanamido)-5-ureidopentanamido)-2-((prop-2-yn-1-yloxy)methyl)benzyl)-3-((R)-2-(2-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-4-chlorobenzoyl)-1,2,3,4-tetrahydroisoquinolin-3-yl)-N,N-dimethylpropan-1-aminium trifluoroacetate

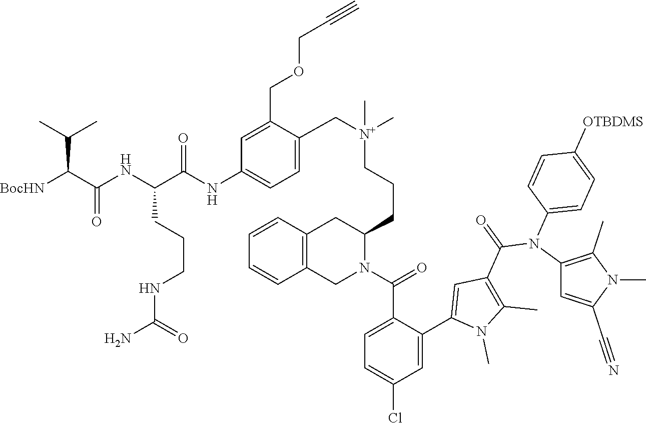 [SMILES: C#CCOCc1cc(NC(=O)[C@H](CCCNC(N)=O)NC(=O)[C@@H](NC(=O)OC(C)(C)C)C(C)C)ccc1C[N+](C)(C)CCC[C@@H]1Cc2ccccc2CN1C(=O)c1ccc(Cl)cc1-c1cc(C(=O)N(c2ccc(O[Si](C)(C)C(C)(C)C)cc2)c2cc(C#N)n(C)c2C)c(C)n1C]

A solution of(R)-N-(4-((tert-butyldimethylsilyl)oxy)phenyl)-5-(5-chloro-2-(3-(3-(dimethylamino)propyl)-1,2,3,4-tetrahydroisoquinoline-2-carbonyl)phenyl)-N-(5-cyano-1,2-dimethyl-1H-pyrrol-3-yl)-1,2-dimethyl-1H-pyrrole-3-carboxamide (400 mg, 0.489 mmol), tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate (0.497 g, 0.685 mmol) and DIEA (426 p L, 2.45 mmol) in DMSO (2 mL) was stirred at rt for 16 hours. The solution was purified by ISCO C18 RP-HPLC. Upon lyophilization, N-(4-((S)-2-((S)-2-((tert-butoxycarbonyl)amino)-3-methylbutanamido)-5-ureidopentanamido)-2-((prop-2-yn-1-yloxy)methyl)benzyl)-3-((R)-2-(2-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-4-chlorobenzoyl)-1,2,3,4-tetrahydroisoquinolin-3-yl)-N,N-dimethylpropan-1-aminium trifluoroacetate (575 mg, 87%) was obtained. HRMS: M⁺=1346.4700, Rt=3.22 min (5 min acidic method).

### Synthesis of N-(4-((S)-2-((S)-2-amino-3-methylbutanamido)-5-ureidopentanamido)-2-((prop-2-yn-1-yloxy)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

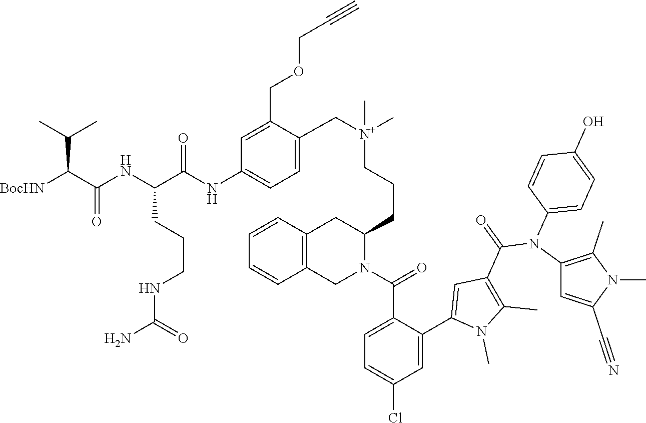 [SMILES: C#CCOCc1cc(NC(=O)[C@H](CCCNC(N)=O)NC(=O)[C@@H](NC(=O)OC(C)(C)C)C(C)C)ccc1C[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C]

To a solution of N-(4-((S)-2-((S)-2-((tert-butoxycarbonyl)amino)-3-methylbutanamido)-5-ureidopentanamido)-2-((prop-2-yn-1-yloxy)methyl)benzyl)-3-((R)-2-(2-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-4-chlorobenzoyl)-1,2,3,4-tetrahydroisoquinolin-3-yl)-N,N-dimethylpropan-1-aminium trifluoroacetate (930 mg, 690 μmol) in MeOH (8 mL) was added HCl(conc.) (1.13 mL, 13.81 mmol). After stirring for 17 hours, the volatiles were removed in vacuo. The residue was dissolved in DMSO, purified by RP-HPLC and after lyophilization, N-(4-((S)-2-((S)-2-amino-3-methylbutanamido)-5-ureidopentanamido)-2-((prop-2-yn-1-yloxy)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (584 mg, 75% yield) was obtained. HRMS: M⁺=1132.4000, Rt=1.90 min (5 min acidic method).

### Synthesis of N-(2-(((((9H-fluoren-9-yl)methoxy)carbonyl)(methyl)amino)methyl)-4-((R)-2-((R)-2-((tert-butoxycarbonyl)amino)-3-methylbutanamido)-5-ureidopentanamido)benzyl)-3-((R)-2-(2-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-4-chlorobenzoyl)-1,2,3,4-tetrahydroisoquinolin-3-yl)-N,N-dimethylpropan-1-aminium trifluoroacetate

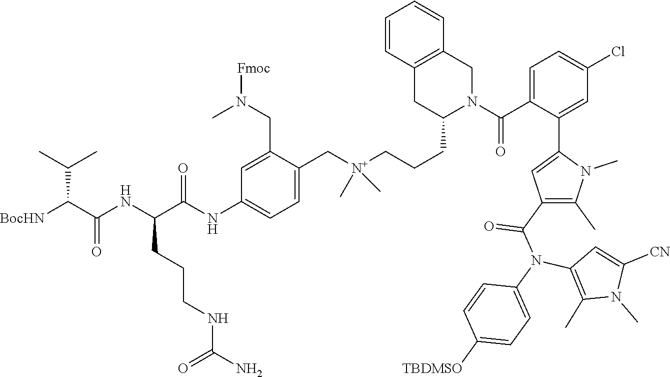

A solution of (R)-N-(4-((tert-butyldimethylsilyl)oxy)phenyl)-5-(5-chloro-2-(3-(3-(dimethylamino)propyl)-1,2,3,4-tetrahydroisoquinoline-2-carbonyl)phenyl)-N-(5-cyano-1,2-dimethyl-1H-pyrrol-3-yl)-1,2-dimethyl-1H-pyrrole-3-carboxamide (1.0 g, 1.224 mmol), (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate (1.214 g, 1.590 mmol), tetrabutylammonium iodide (496 mg, 1.346 mmol) and DIEA (474 mg, 3.67 mmol) in DMSO (6 mL) was stirred at rt for 2 hours. The solution was purified by ISCO C18 RP-HPLC. Upon lyophilization, N-(2-(((((9H-fluoren-9-yl)methoxy)carbonyl)(methyl)amino)methyl)-4-((R)-2-((R)-2-((tert-butoxycarbonyl)amino)-3-methylbutanamido)-5-ureidopentanamido)benzyl)-3-((R)-2-(2-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-4-chlorobenzoyl)-1,2,3,4-tetrahydroisoquinolin-3-yl)-N,N-dimethylpropan-1-aminium trifluoroacetate (1.414 g, 74%) was obtained. HRMS: M⁺=1543.8101, Rt=2.64 min (5 min acidic method).

### Synthesis of N-(2-(((((9H-fluoren-9-yl)methoxy)carbonyl)(methyl)amino)methyl)-4-((R)-2-((R)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifuoroacetate

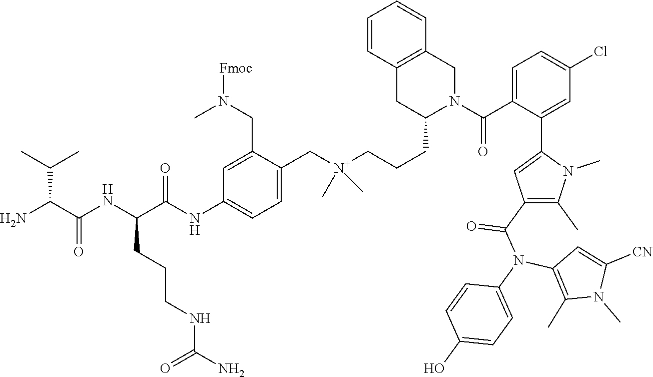

To a solution of N-(2-(((((9H-fluoren-9-yl)methoxy)carbonyl)(methyl)amino)methyl)-4-((R)-2-((R)-2-((tert-butoxycarbonyl)amino)-3-methylbutanamido)-5-ureidopentanamido)benzyl)-3-((R)-2-(2-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-4-chlorobenzoyl)-1,2,3,4-tetrahydroisoquinolin-3-yl)-N,N-dimethylpropan-1-aminium trifluoroacetate (952 mg, 0.574 mmol) in EtOH (4 mL) was added 4 M HCl in dioxane (0.94 mL, 11.5 mmol). After stirring for 3 hours, the volatiles were removed in vacuo, the residue was dissolved in DMSO and was purified by ISCO C18 RP-HPLC. Upon lyophilization, N-(2-(((((9H-fluoren-9-yl)methoxy)carbonyl)(methyl)amino)methyl)-4-((R)-2-((R)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifuoroacetate (664 mg, 81%) was obtained. HRMS: M+=1329.6700, Rt=2.07 min (5 min acidic method).

### Synthesis of N-(4-((R)-2-((R)-2-(2-azidoacetamido)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

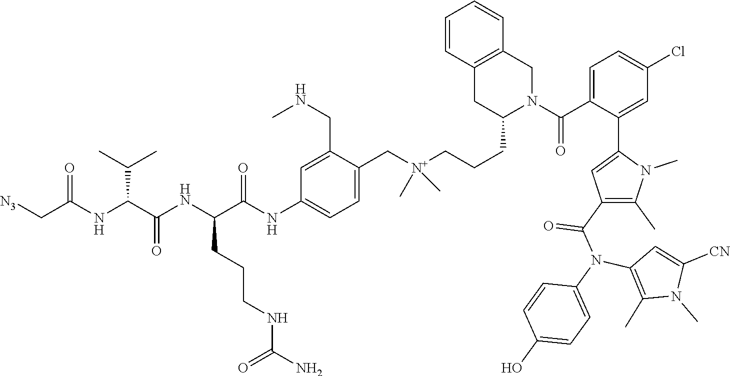 [SMILES: CNCc1cc(NC(=O)[C@@H](CCCNC(N)=O)NC(=O)[C@H](NC(=O)CN=[N+]=[N-])C(C)C)ccc1C[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C]

To a solution of N-(2-(((((9H-fluoren-9-yl)methoxy)carbonyl)(methyl)amino)methyl)-4-((S)-2-((S)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (150 mg, 96 μmol) and 2,5-dioxopyrrolidin-1-yl 2-azidoacetate (22 mg, 111 Ipmol) in DMF (2 mL) was added DIEA (50 μL, 289 μmol). After stirring for 20 hours, 2M Me₂NH in THF (240 μL, 480 μmol) was added. After stirring an additional one hour, the solution was diluted with DMSO and purified by RP-HPLC. After lyophilization, N-(4-((R)-2-((R)-2-(2-azidoacetamido)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (93 mg, 68% yield) was obtained. HRMS: M⁺=1190.5699, Rt=1.70 min (5 min acidic method).

### Synthesis of N-(4-((S)-2-((S)-2-(((allyloxy)carbonyl)amino)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

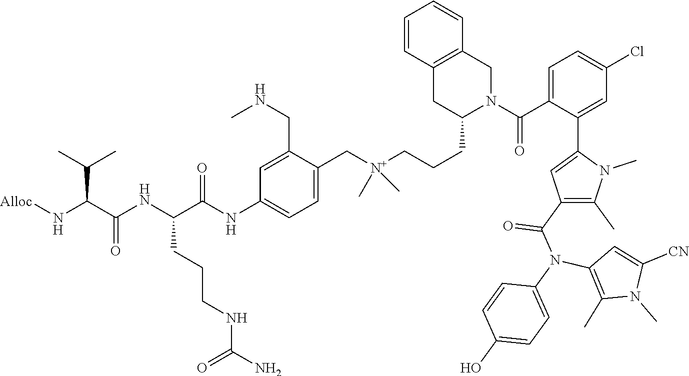

To a solution of N-(2-(((((9H-fluoren-9-yl)methoxy)carbonyl)(methyl)amino)methyl)-4-((R)-2-((R)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifuoroacetate (316 mg, 0.22 mmol) and N-allylcarbonyloxy succinimide (87 mg, 0.437) in DMF (1 mL) was added DIEA (141 mg, 1.09 mmol). After stirring for 2 hours, 2.0 M Dimethyl amine in MeOH (1.09 mL, 2.18 mmoL) was added. After stirring for 45 minutes, the solution was diluted with DMSO and purified by ISCO RP-HPLC. Upon lyophilization, N-(4-((S)-2-((S)-2-(((allyloxy)carbonyl)amino)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (208 mg, 79% yield) was obtained. HRMS: M⁺=1191.6100, Rt=1.83 min (5 min acidic method).

### Synthesis of N-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

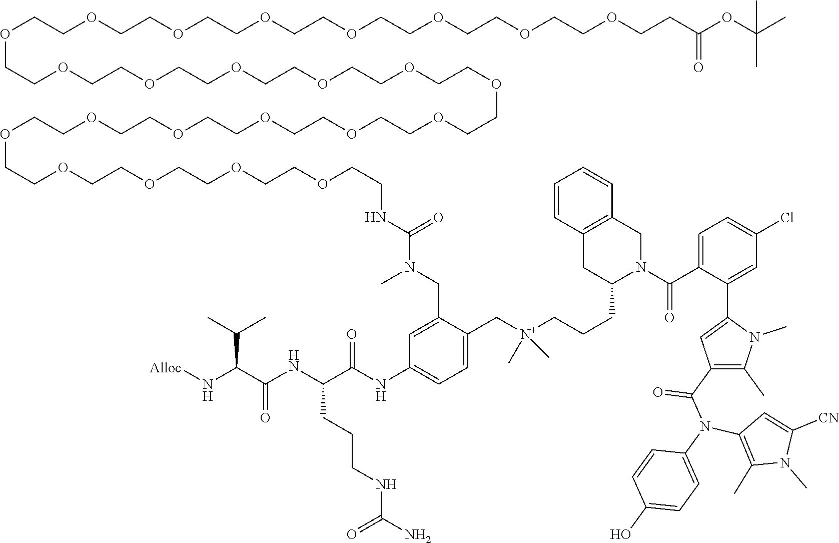

GENERAL PROCEDURE #1: to a solution of bis(4-nitrophenyl) carbonate (50.6 mg, 166 μmol), tert-butyl 1-amino-3,6,9,12,15,18,21,24,27,30,33,36,39,42,45,48,51,54,57,60,63,66,69,72-tetracosaoxapentaheptacontan-75-oate (200 mg, 166 μmol) in DMF (2 mL) was added DIEA (58 μL, 333 μmol). After stirring for 1 hour, N-(4-((S)-2-((S)-2-(((allyloxy)carbonyl)amino)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (207 mg, 158 μmol) was added to the solution as well as additional DIEA (58 μL, 333 μmol). After stirring for an additional 2 hours, the solution was diluted with DMSO and purified by ISCO RP-HPLC. Upon lyophilization, N-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (335 mg, 84% yield) was obtained. HRMS: (M⁺−H⁺+Na⁺)⁺=2441.2800, Rt=2.46 min (5 min acidic method).

### Synthesis of N-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

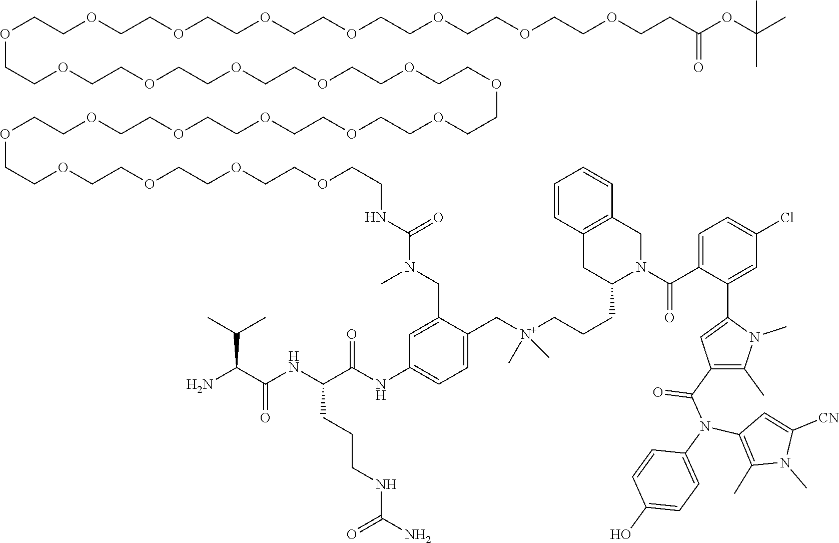 [SMILES: Cc1c(N(C(=O)c2cc(-c3cc(Cl)ccc3C(=O)N3Cc4ccccc4C[C@H]3CCC[N+](C)(C)Cc3ccc(NC(=O)[C@H](CCCNC(N)=O)NC(=O)[C@@H](N)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)n(C)c2C)c2ccc(O)cc2)cc(C#N)n1C]

GENERAL PROCEDURE #2: to a solution of N-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (214 mg, 84 μmol) in THF (6 mL) was added N,N,1,1,1-pentamethylsilanamine (59 mg, 505 μmol). An N₂ balloon with 3-way stopcock was attached and house vacuum was pulled and reaction purged to N₂ (repeated 2 ×). Tetrakis(triphenylphosphine)palladium (19.5 mg, 17 μmol) was added, followed by degassing/purging to N₂ as above (3×). After stirring for one hour, the volatiles were removed in vacuo, the residue was dissolved in DMSO and purified by C18 RP-ISCO. After lyophilization, N-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate was obtained (134 mg, 62%). HRMS: M⁺=2335.3000, Rt=2.17 min (5 min acidic method).

Variations of this general method included reducing the equivalents of tetrakis(triphenylphosphine)palladium used, use of alternative amine nucleophiles (pyrrolidine, morpholine) as well as running the reaction in 1:1 CH₂Cl₂/MeOH with the omission of an amine nucleophile specifically in cases when an FMOC was present.

### Synthesis of 4-(4-((S)-2-((S)-2-(((allyloxy)carbonyl)amino)-3-methylbutanamido)-5-ureidopentanamido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

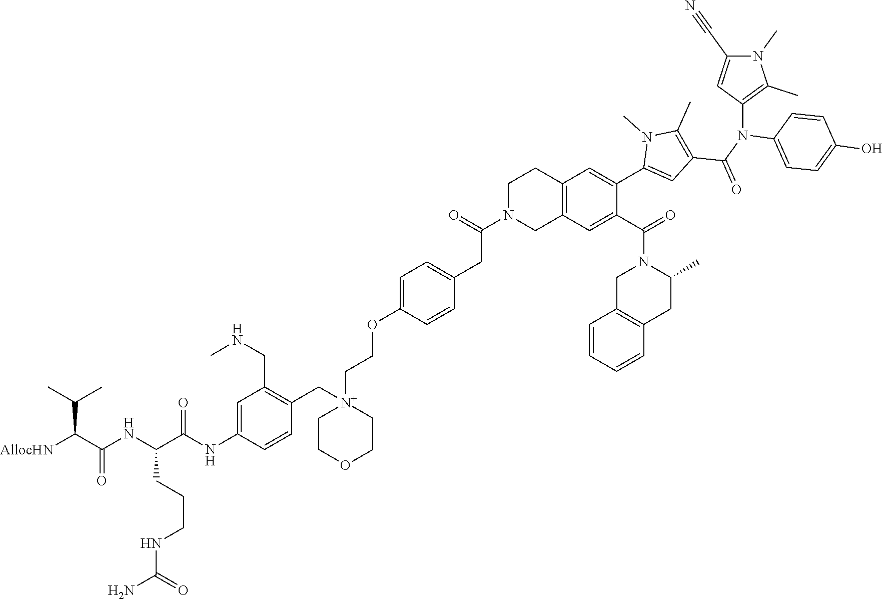

A solution of (R)-N-(4-((tert-butyldimethylsilyl)oxy)phenyl)-N-(5-cyano-1,2-dimethyl-1H-pyrrol-3-yl)-1,2-dimethyl-5-(7-(3-methyl-1,2,3,4-tetrahydroisoquinoline-2-carbonyl)-2-(2-(4-(2-morpholinoethoxy)phenyl)acetyl)-1,2,3,4-tetrahydroisoquinolin-6-yl)-1H-pyrrole-3-carboxamide (600 mg, 592 μmol), (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate (796 mg, 1060 μmol), tetrabutylammonium iodide (218 mg, 592 μmol) and DIEA (103 μL, 592 μmol) in DMF (1.8 mL) was stirred at rt for 40 hours. The solution was purified by ISCO C18 RP-HPLC. After lyophilization, the residue (630 mg) was dissolved in DMF (2 mL) and 2M dimethyl amine in MeOH (1.77 mL, ca. 3400 μmol) was added. After standing two hours, 1.0 M tetrabutyl ammonium fluoride (0.20 mL, 200 μmol) was added. After 30 minutes, the solution was diluted in DMSO and purified by C18 ISCO RP-HPLC. Upon lyophilization, 4-(4-((S)-2-((S)-2-(((allyloxy)carbonyl)amino)-3-methylbutanamido)-5-ureidopentanamido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (515 mg, 51%) was obtained. HRMS: M⁺=1388.7200, Rt=1.83 min (5 min acidic method).

### Synthesis of 4-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

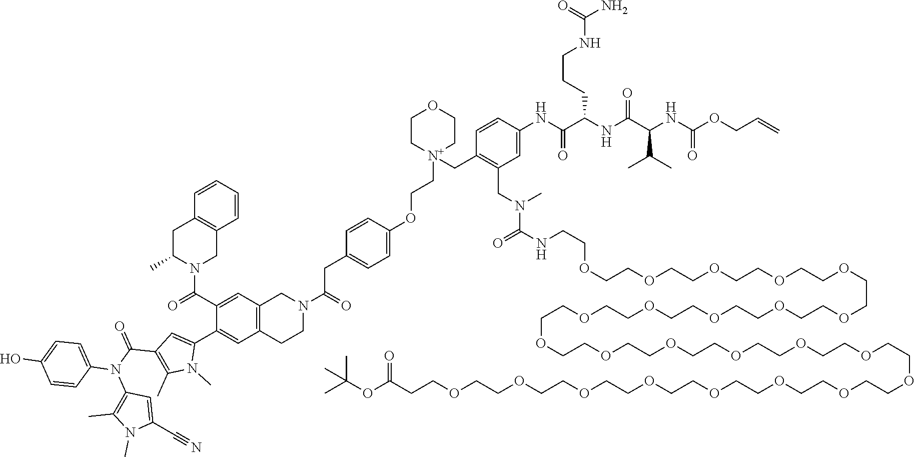 [SMILES: C=CCOC(=O)N[C@H](C(=O)N[C@@H](CCCNC(N)=O)C(=O)Nc1ccc(C[N+]2(CCOc3ccc(CC(=O)N4CCc5cc(-c6cc(C(=O)N(c7ccc(O)cc7)c7cc(C#N)n(C)c7C)c(C)n6C)c(C(=O)N6Cc7ccccc7C[C@H]6C)cc5C4)cc3)CCOCC2)c(CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)c1)C(C)C]

Following GENERAL PROCEDURE #1 using bis(4-nitrophenyl) carbonate (92.6 mg, 303.4 μmol), tert-butyl 1-amino-3,6,9,12,15,18,21,24,27,30,33,36,39,42,45,48,51,54,57,60,63,66,69,72-tetracosaoxapentaheptacontan-75-oate (366 mg, 304.4 μmol), DIEA (110 μL, 634 μmol) and then 4-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium Trifluoroacetate (513 mg, 317 μmol) and DIEA (110 μL, 634 μmol), 4-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium Trifluoroacetate (688 mg, 79%) was obtained. HRMS: M⁺=2616.4500, Rt=2.42 min (5 min acidic method).

### Synthesis of 4-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

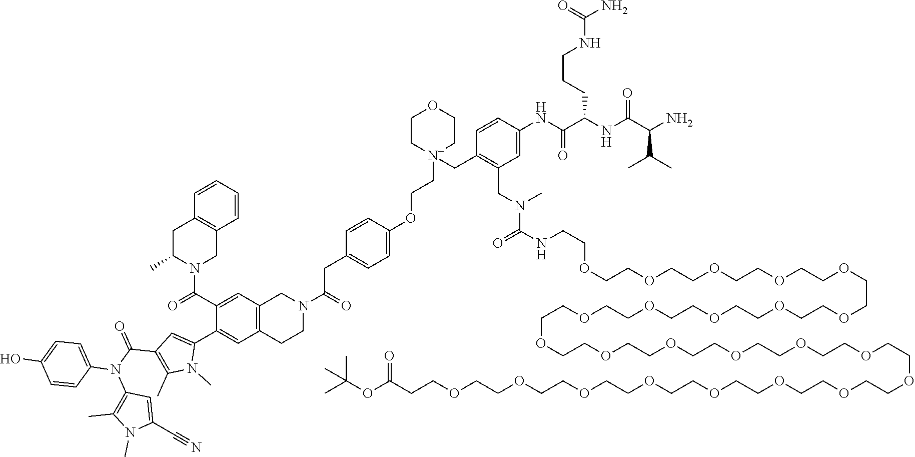 [SMILES: Cc1c(N(C(=O)c2cc(-c3cc4c(cc3C(=O)N3Cc5ccccc5C[C@H]3C)CN(C(=O)Cc3ccc(OCC[N+]5(Cc6ccc(NC(=O)[C@H](CCCNC(N)=O)NC(=O)[C@@H](N)C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)CCOCC5)cc3)CC4)n(C)c2C)c2ccc(O)cc2)cc(C#N)n1C]

Following GENERAL PROCEDURE #2 using 4-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (688 mg, 251.8 μmol), N,N,1,1,1-pentamethylsilanamine (242 μL, 1511 μmol) and tetrakis(triphenylphosphine)palladium (14.5 mg, 12.7 μmol), 4-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate was obtained (486 mg, 69%). HRMS: M⁺=2532.4099, Rt=2.15 min (5 min acidic method).

### Synthesis of 4-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(2-(4-(2-(6-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

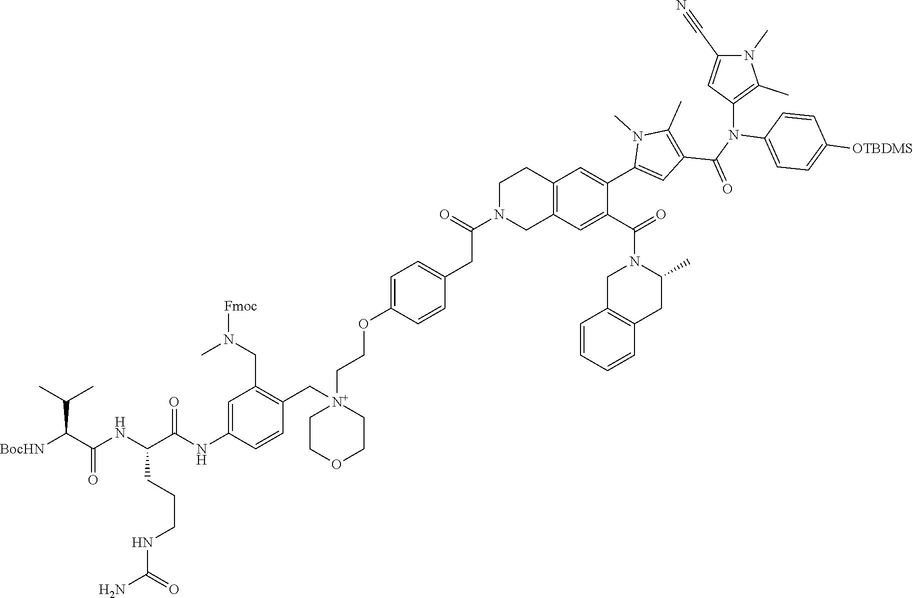

A solution of (R)-N-(4-((tert-butyldimethylsilyl)oxy)phenyl)-N-(5-cyano-1,2-dimethyl-1H-pyrrol-3-yl)-1,2-dimethyl-5-(7-(3-methyl-1,2,3,4-tetrahydroisoquinoline-2-carbonyl)-2-(2-(4-(2-morpholinoethoxy)phenyl)acetyl)-1,2,3,4-tetrahydroisoquinolin-6-yl)-1H-pyrrole-3-carboxamide (200 mg, 197 μmol), (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate (196 mg, 257 μmol) and sodium iodide (35.5 mg, 237 μmol) in DMSO (1 mL) was heated at 30° C. for 36 hours. Upon cooling, the solution was purified by ISCO C18 RP-HPLC. After lyophilization, 4-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(2-(4-(2-(6-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium Trifluoroacetate (249 mg, 67%) was obtained. HRMS: M⁺=1740.9100, Rt=3.26 min (5 min acidic method).

### Synthesis of 4-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

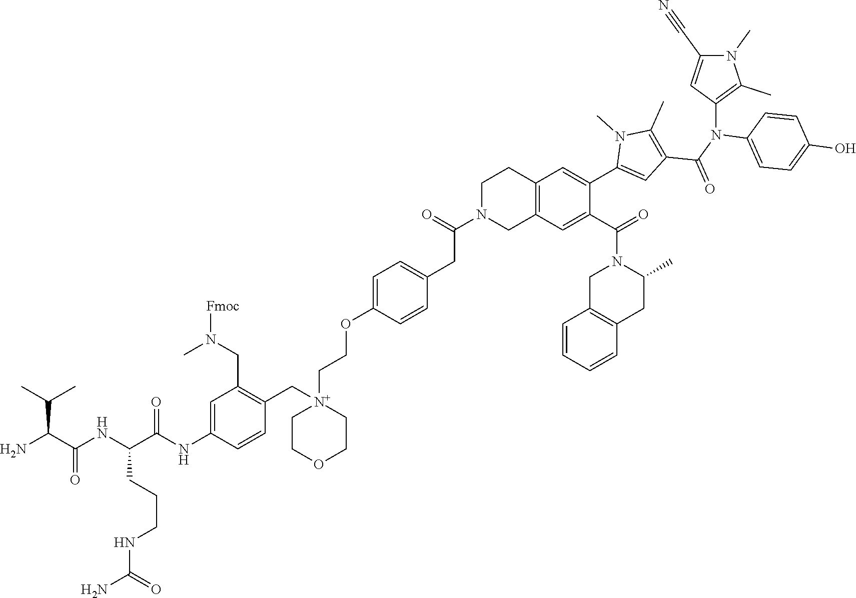

A solution of 4-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(2-(4-(2-(6-(4-((4-((tert-butyldimethylsilyl)oxy)phenyl)(5-cyano-1,2-dimethyl-1H-pyrrol-3-yl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (308 mg, 165.9 μmol) in MeOH (3 mL) was cooled in ice bath and 4M HCl in dioxane (3 mL, 12 mmol) was added. After stirring for one hour, the volatiles were removed in vacuo. The residue was dissolved in DMSO and purified by ISCO C18 RP-HPLC. After lyophilization, 4-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium Trifluoroacetate was obtained (170 mg, 58%). HRMS: M⁺=1526.8101, Rt=2.02 min (5 min acidic method).

### Synthesis of 4-(4-((S)-2-((S)-2-(((allyloxy)carbonyl)amino)-3-methylbutanamido)-5-ureidopentanamido)-2-((methylamino)methyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

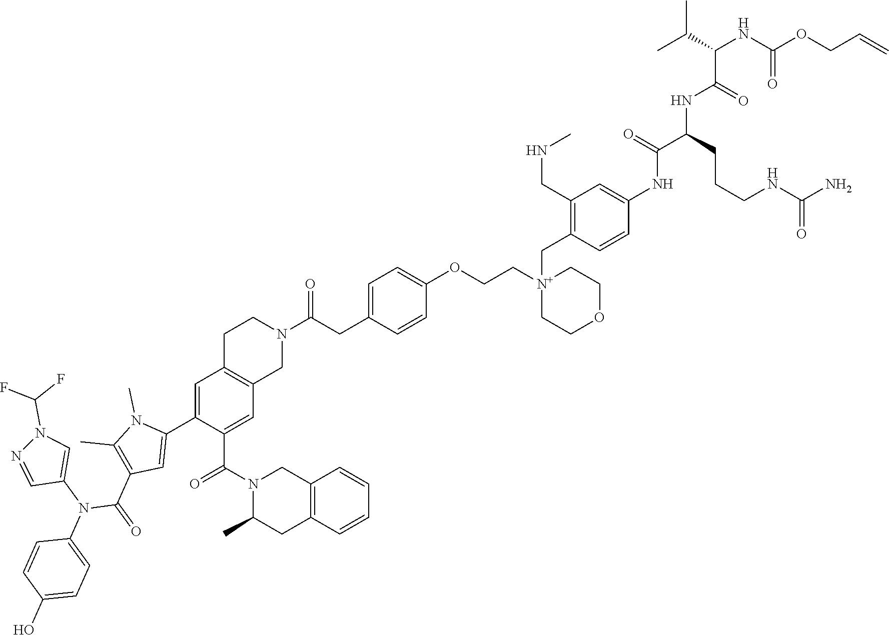 [SMILES: C=CCOC(=O)N[C@H](C(=O)N[C@@H](CCCNC(N)=O)C(=O)Nc1ccc(C[N+]2(CCOc3ccc(CC(=O)N4CCc5cc(-c6cc(C(=O)N(c7ccc(O)cc7)c7cnn(C(F)F)c7)c(C)n6C)c(C(=O)N6Cc7ccccc7C[C@H]6C)cc5C4)cc3)CCOCC2)c(CNC)c1)C(C)C]

A solution of (R)-N-(4-((tert-butyldimethylsilyl)oxy)phenyl)-N-(1-(difluoromethyl)-1H-pyrazol-4-yl)-1,2-dimethyl-5-(7-(3-methyl-1,2,3,4-tetrahydroisoquinoline-2-carbonyl)-2-(2-(4-(2-morpholinoethoxy)phenyl)acetyl)-1,2,3,4-tetrahydroisoquinolin-6-yl)-1H-pyrrole-3-carboxamide (600 mg, 593 μmol), (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate (930 mg, 1245 μmol), tetrabutylammonium iodide (219 mg, 593 μmol) and DIEA (108 p L, 622 μmol) in DMF (2.1 mL) was stirred at rt for 60 hours. The solution was purified by ISCO C18 RP-HPLC. After lyophilization, the residue (630 mg) was dissolved in DMF (2 mL) and 2M dimethyl amine in MeOH (2 mL, ca. 4000 μmol) was added. After standing two hours, 1.0 M tetrabutyl ammonium fluoride (0.20 mL, 200 μmol) was added. After 60 minutes, the solution was diluted in DMSO and purified by C18 ISCO RP-HPLC. Upon lyophilization, 4-(4-((S)-2-((S)-2-(((allyloxy)carbonyl)amino)-3-methylbutanamido)-5-ureidopentanamido)-2-((methylamino)methyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (518 mg, 52%) was obtained. HRMS: M⁺=1386.6899, Rt=1.83 min (5 min acidic method).

### Synthesis of 4-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

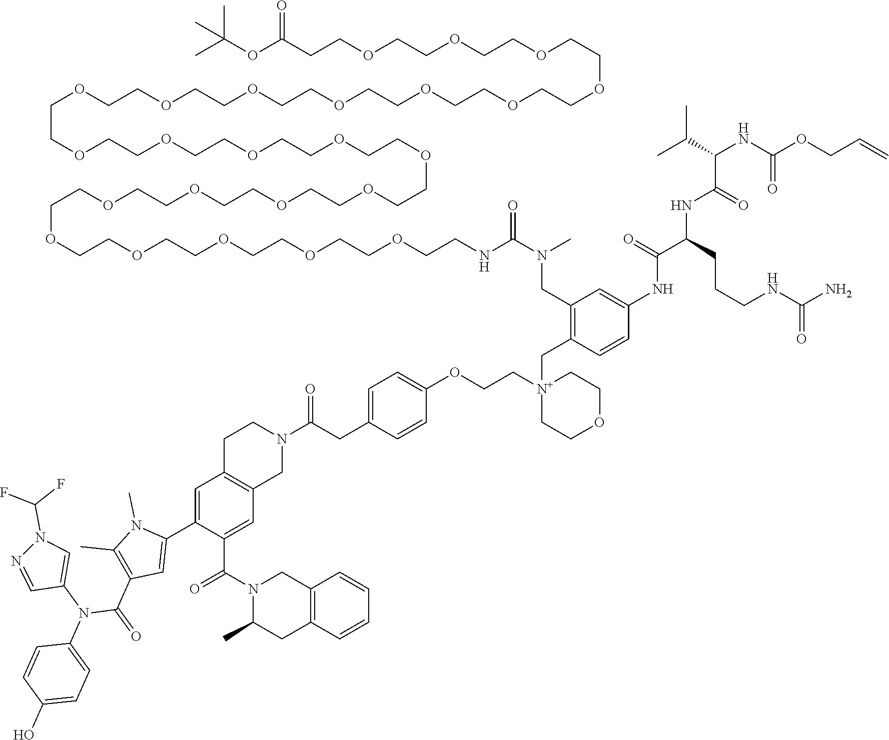 [SMILES: C=CCOC(=O)N[C@H](C(=O)N[C@@H](CCCNC(N)=O)C(=O)Nc1ccc(C[N+]2(CCOc3ccc(CC(=O)N4CCc5cc(-c6cc(C(=O)N(c7ccc(O)cc7)c7cnn(C(F)F)c7)c(C)n6C)c(C(=O)N6Cc7ccccc7C[C@H]6C)cc5C4)cc3)CCOCC2)c(CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)c1)C(C)C]

Following GENERAL PROCEDURE #1 using bis(4-nitrophenyl) carbonate (93.1 mg, 306 μmol), DIEA (11 p L, 637.5 μmol), ), tert-butyl 1-amino-3,6,9,12,15,18,21,24,27,30,33,36,39,42,45,48,51,54,57,60,63,66,69,72-tetracosaoxapentaheptacontan-75-oate (368 mg, 306 μmol) and then 4-(4-((S)-2-((S)-2-(((allyloxy)carbonyl)amino)-3-methylbutanamido)-5-ureidopentanamido)-2-((methylamino)methyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium (515 mg, 319 μmol) and additional DIEA (111 μL, 637.5 mmol), 4-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (696 mg, 79%) was obtained. HRMS: M⁺=2614.4099, Rt=2.47 min (5 min acidic method

### Synthesis of 4-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

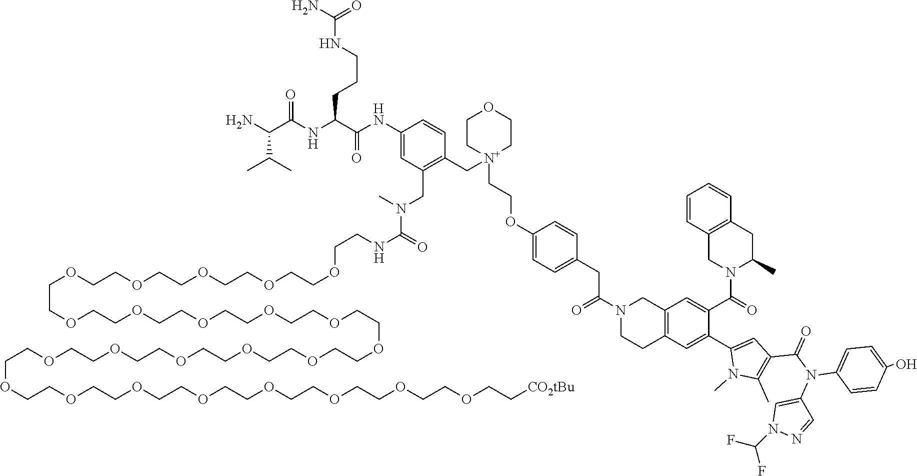 [SMILES: Cc1c(C(=O)N(c2ccc(O)cc2)c2cnn(C(F)F)c2)cc(-c2cc3c(cc2C(=O)N2Cc4ccccc4C[C@H]2C)CN(C(=O)Cc2ccc(OCC[N+]4(Cc5ccc(NC(=O)[C@H](CCCNC(N)=O)NC(=O)[C@@H](N)C(C)C)cc5CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)CCOCC4)cc2)CC3)n1C]

Following GENERAL PROCEDURE #2 using 4-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (696 mg, 255 μmol), N,N,1,1,1-pentamethylsilanamine (245 μL, 1529 μmol) and tetrakis(triphenylphosphine)palladium (20.6 mg, 17.8 μmol) was, 4-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (452 mg, 64% yield) was obtained. HRMS: M⁺=2530.3899, Rt=2.24 min (5 min acidic method).

### Synthesis of 3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N-(4-((10S,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-((methylamino)methyl)benzyl)-N,N-dimethylpropan-1-aminium trifluoroacetate

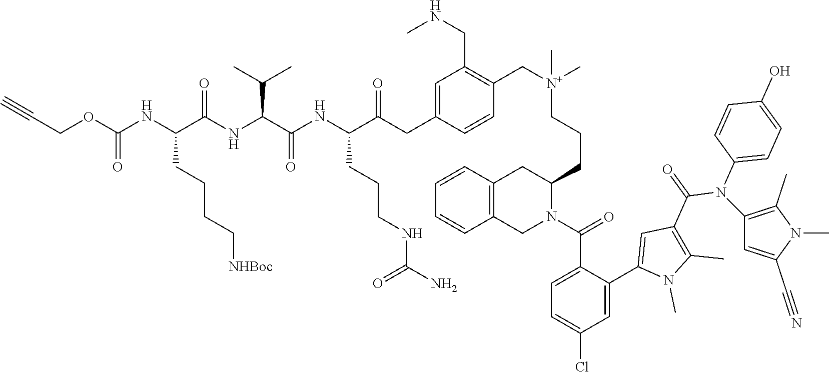 [SMILES: C#CCOC(=O)N[C@@H](CCCCNC(=O)OC(C)(C)C)C(=O)N[C@H](C(=O)N[C@@H](CCCNC(N)=O)C(=O)Cc1ccc(C[N+](C)(C)CCC[C@@H]2Cc3ccccc3CN2C(=O)c2ccc(Cl)cc2-c2cc(C(=O)N(c3ccc(O)cc3)c3cc(C#N)n(C)c3C)c(C)n2C)c(CNC)c1)C(C)C]

To a solution of N6-(tert-butoxycarbonyl)-N2-((prop-2-yn-1-yloxy)carbonyl)-L-lysine (31.6 mg, 96 μmol) and HATU (40.2 mg, 106 μmol) in DMF (1 mL) was added DIEA (67 μL, 185 μmol). After stirring for 5 minutes, N-(2-(((((9H-fluoren-9-yl)methoxy)carbonyl)(methyl)amino)methyl)-4-((S)-2-((S)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (150 mg, 96 μmol) was added. After stirring for an additional four hours, 2.0 M Me₂NH in THF (1.44 mL, 2880 μmol) was added. After stirring for 2 hours, the volatiles were removed in vacuo, the residue was diluted with DMSO and purified by ISCO RP-HPLC. Upon lyophilization, 3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N-(4-((1oS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-((methylamino)methyl)benzyl)-N,N-dimethylpropan-1-aminium trifluoroacetate (103 mg, 76% yield) was obtained. HRMS: M⁺=1417.6700, Rt=1.95 min (5 min acidic method).

### Synthesis of N-(4-((S)-2-((S)-2-(((2-azidoethoxy)carbonyl)amino)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

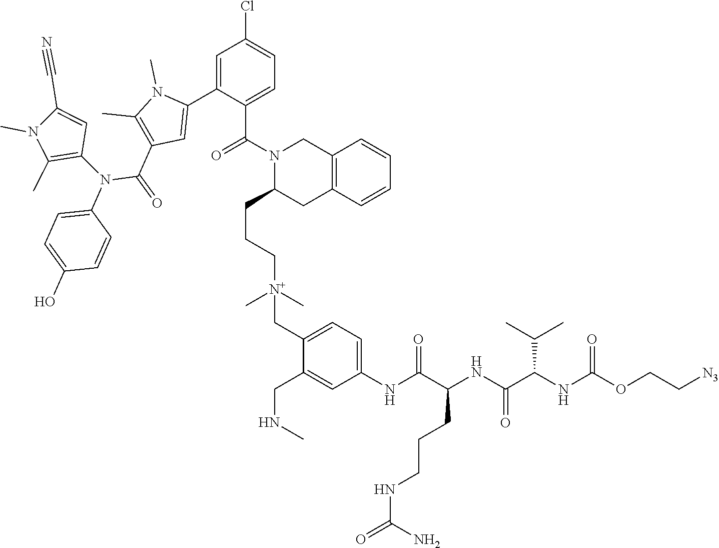 [SMILES: CNCc1cc(NC(=O)[C@H](CCCNC(N)=O)NC(=O)[C@@H](NC(=O)OCCN=[N+]=[N-])C(C)C)ccc1C[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C]

A solution of (R)-N-(4-((tert-butyldimethylsilyl)oxy)phenyl)-5-(5-chloro-2-(3-(3-(dimethylamino)propyl)-1,2,3,4-tetrahydroisoquinoline-2-carbonyl)phenyl)-N-(5-cyano-1,2-dimethyl-1H-pyrrol-3-yl)-1,2-dimethyl-1H-pyrrole-3-carboxamide (560 mg, 685 μmol), (9H-fluoren-9-yl)methyl (5-((S)-2-((S)-2-(((2-azidoethoxy)carbonyl)amino)-3-methylbutanamido)-5-ureidopentanamido)-2-(chloromethyl)benzyl)(methyl)carbamate (617 mg, 795 μmol), tetrabutylammonium iodide (76 mg, 205 μmol) and DIEA (239 μL, 1370 μmol) in DMF (7 mL) was stirred at rt for 14 hours at which time 2M Me₂NH (1.712 mL, 3424 μmol) was added. After stirring an additional 2 hours, the volatiles were removed in vacuo, DMSO was added and the solution was purified by ISCO C18 RP-HPLC. Upon lyophilization, N-(4-((S)-2-((S)-2-(((2-azidoethoxy)carbonyl)amino)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (895 mg, 98%) was obtained. HRMS: M⁺=1220.5778, Rt=1.78 min (5 min acidic method).

### Synthesis of N-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

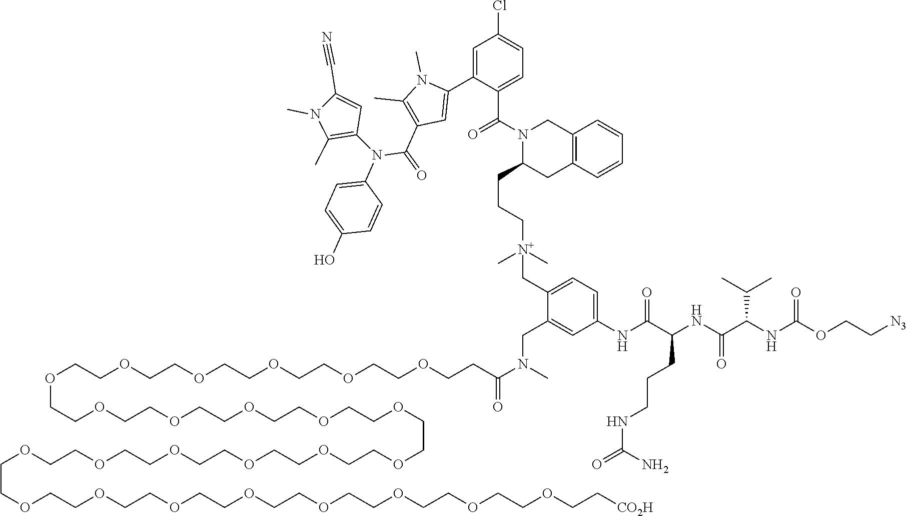 [SMILES: Cc1c(N(C(=O)c2cc(-c3cc(Cl)ccc3C(=O)N3Cc4ccccc4C[C@H]3CCC[N+](C)(C)Cc3ccc(NC(=O)[C@H](CCCNC(N)=O)NC(=O)[C@@H](NC(=O)OCCN=[N+]=[N-])C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)n(C)c2C)c2ccc(O)cc2)cc(C#N)n1C]

To a solution of N-(4-((S)-2-((S)-2-(((2-azidoethoxy)carbonyl)amino)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (110 mg, 82 μmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (119 mg, 97 μmol) in DMF (1.2 mL) was added DIEA (72 p L, 412 μmol). After stirring for 16 hours, the solution was diluted with DMSO and purified by RP-HPLC. After lyophilization, N-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (183 mg, 88% yield) was obtained. HRMS: M⁺=2421.2200, Rt=2.16 min (5 min acidic method).

### Synthesis of N-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

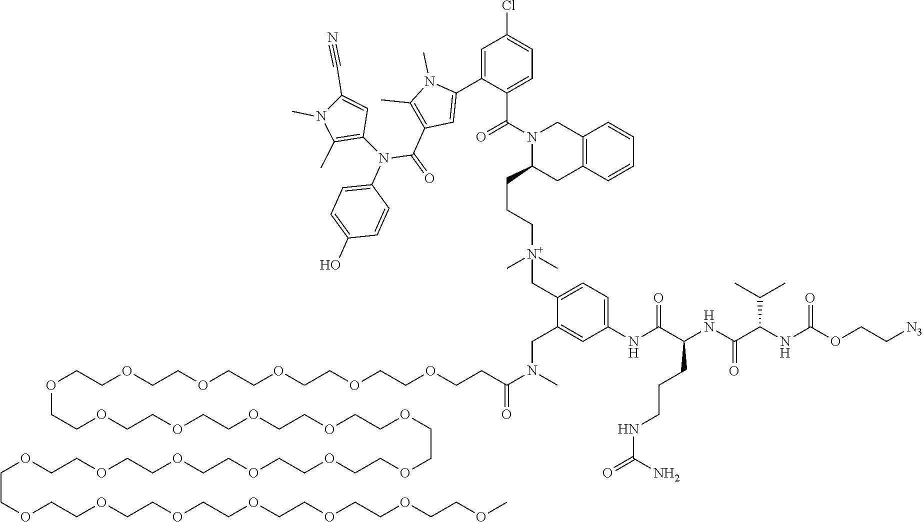 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)OCCN=[N+]=[N-])C(C)C)ccc1C[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C]

To a solution of N-(4-((S)-2-((S)-2-(((2-azidoethoxy)carbonyl)amino)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (150 mg, 112 μmol) and 2,5-dioxopyrrolidin-1-yl 2,5,8,11,14,17,20,23,26,29,32,35,38,41,44,47,50,53,56,59,62,65,68,71-tetracosaoxatetraheptacontan-74-oate (150 mg, 124 μmol) in DMF (1.6 mL) was added DIEA (98 μL, 561 μmol). After stirring for 16 hours, the solution was diluted with DMSO and purified by RP-HPLC. After lyophilization, N-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (209 mg, 76% yield) was obtained. HRMS: M⁺=2319.2100, Rt=2.23 min (5 min acidic method).

### Synthesis of N-(4-((9S,12S)-1-azido-9-isopropyl-4,7,10-trioxo-12-(3-ureidopropyl)-3-oxa-5,8,11-triazatridecan-13-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

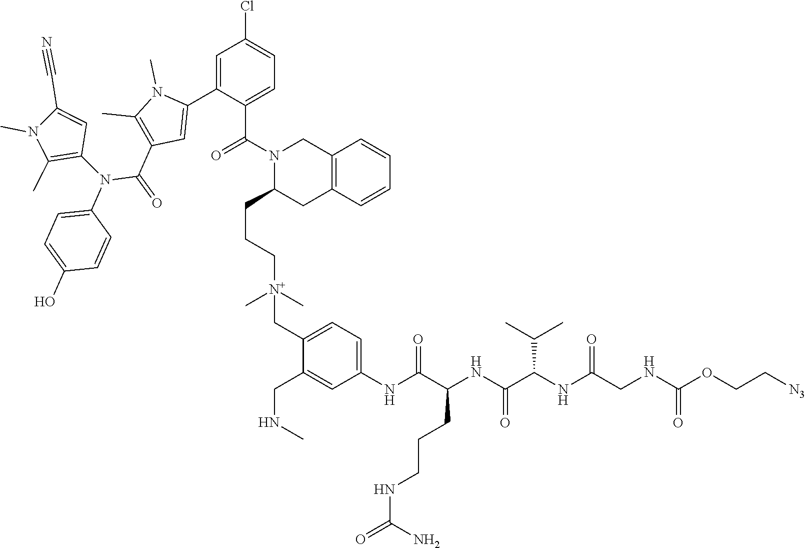 [SMILES: CNCc1cc(NC(=O)[C@H](CCCNC(N)=O)NC(=O)[C@@H](NC(=O)CNC(=O)OCCN=[N+]=[N-])C(C)C)ccc1C[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C]

To a solution of N-(2-(((((9H-fluoren-9-yl)methoxy)carbonyl)(methyl)amino)methyl)-4-((S)-2-((S)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (150 mg, 96 μmol), (2-azidoethoxy)carbonyl)glycine (31 mg, 165 μmol), HBTU (62 mg, 163 μL) in DMF (3 mL) was added DIEA (70 μL, 401 μmol). After stirring for 3 hours, 2M Me₂NH in MeOH (600 μL, 1200 μmol) was added. After stirring for an additional two hours, the solution was diluted with DMSO and purified by RP-HPLC. After lyophilization, N-(4-((9S,12S)-1-azido-9-isopropyl-4,7,10-trioxo-12-(3-ureidopropyl)-3-oxa-5,8,11-triazatridecan-13-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (116 mg, 80% yield) was obtained. HRMS: M⁺=1277.6000, Rt=1.70 min (5 min acidic method).

### Synthesis of 1-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-iumtrifluoroacetate

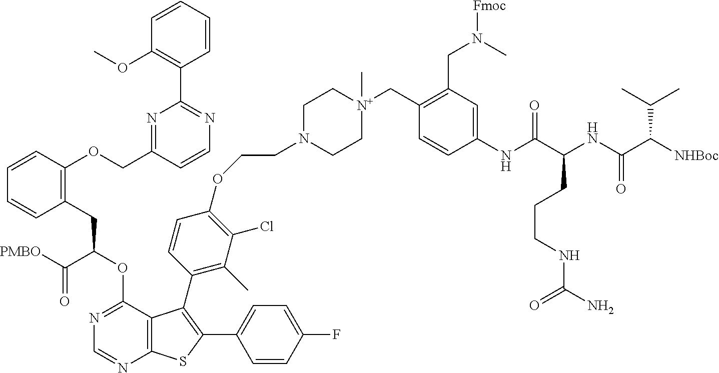

A solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (1000 mg, 1004 μmol, prepared based on methods described in WO2017/125224), (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate (843 mg, 1105 μmol), tetrabutylammonium iodide (408 mg, 1105 μmol) and DIEA (175 u, 1004 μmol) in DMSO (10 mL) was stirred at rt for 3 hours at which time the solution was purified by ISCO RP-HPLC. Upon lyophilization, 1-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (1.45 g, 84%) was obtained. HRMS: M⁺=1721.7100, Rt=3.22 min (5 min acidic method).

### Synthesis of 1-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

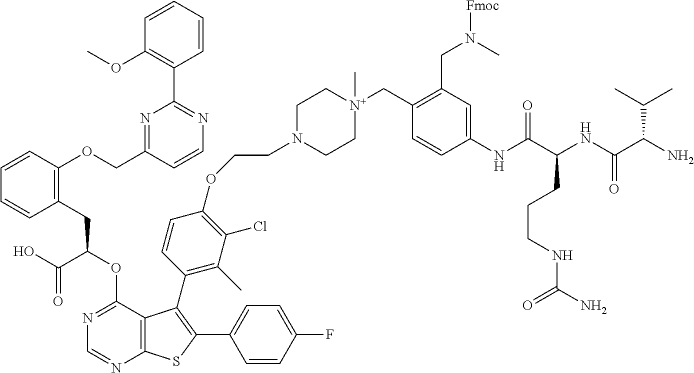

1-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (300 mg, 174 μmol) was treated with 25% TFA/CH₂Cl₂ (4 mL) for 1 hour at which time the volatiles were removed in vacuo. The residue was dissolved in MeCN/H₂O and lyophilized to yield 1-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (305 mg, 95% yield). The material was used directly. LC/MS: [(M⁺)+(H⁺)+²]/2=751.6, Rt=2.30 min (5 min acidic method).

### Synthesis of 1-(4-((S)-2-((S)-2-(((2-azidoethoxy)carbonyl)amino)-3-methylbutanamido)-5-ureidopentanamido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

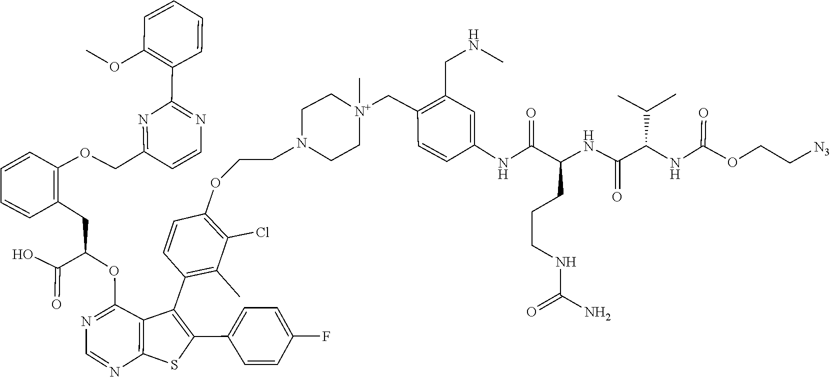 [SMILES: CNCc1cc(NC(=O)[C@H](CCCNC(N)=O)NC(=O)[C@@H](NC(=O)OCCN=[N+]=[N-])C(C)C)ccc1C[N+]1(C)CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1]

To a solution of 1-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (186 mg, 124 μmol) and 2-azidoethyl (4-nitrophenyl) carbonate (62.5 mg, 248 μmol) in DMF (1.3 mL) was added DIEA (97 μL, 560 μmol). After stirring for 2 hours, 2M Me₂NH in MeOH (600 μL, 1200 μmol) was added. After stirring for 2 hours, the solution was diluted with DMSO and purified by RP-HPLC. Upon lyophilization, 1-(4-((S)-2-((S)-2-(((2-azidoethoxy)carbonyl)amino)-3-methylbutanamido)-5-ureidopentanamido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (73 mg, 42% yield) was obtained. LC/MS: [(M⁺+H⁺)+²]/2=697.3, Rt=2.09 min (5 min acidic method).

### Synthesis of 1-(2-(((((9H-fluoren-9-yl)methoxy)carbonyl)(methyl)amino)methyl)-4-((9S,12S)-1-azido-9-isopropyl-4,7,10-trioxo-12-(3-ureidopropyl)-3-oxa-5,8,11-triazatridecan-13-amido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

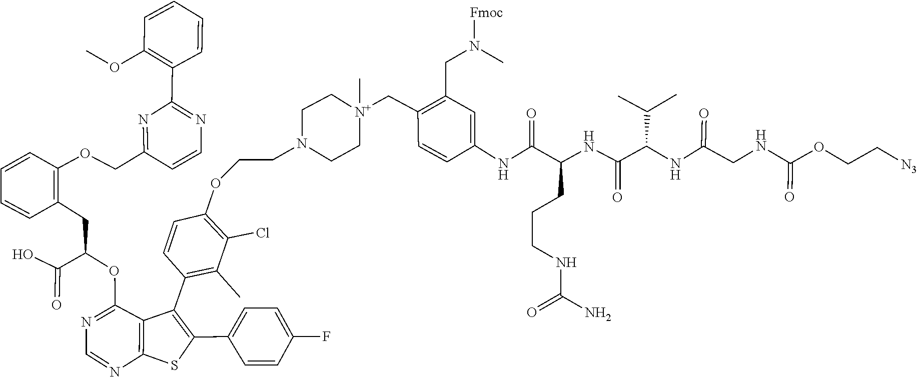

To a solution of ((2-azidoethoxy)carbonyl)glycine (15.3 mg, 81 μmol) and HATU (20.6 mg, 54 μmol) in DMF (2 mL) was added DIEA (38 μL, 217 μmol). After stirring for 5 minutes, 1-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (100 mg, 54 μmol) was added. After stirring for an additional 30 minutes, the solution was diluted with DMSO and purified by ISCO RP-HPLC. Upon lyophilization, 1-(2-(((((9H-fluoren-9-yl)methoxy)carbonyl)(methyl)amino)methyl)-4-((9S,12S)-1-azido-9-isopropyl-4,7,10-trioxo-12-(3-ureidopropyl)-3-oxa-5,8,11-triazatridecan-13-amido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (56 mg, 58% yield) was obtained. HRMS: M⁺=1671.6500, Rt=2.50 min (5 min acidic method).

### Synthesis of 1-(4-((9S,12S)-1-azido-9-isopropyl-4,7,10-trioxo-12-(3-ureidopropyl)-3-oxa-5,8,11-triazatridecan-13-amido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

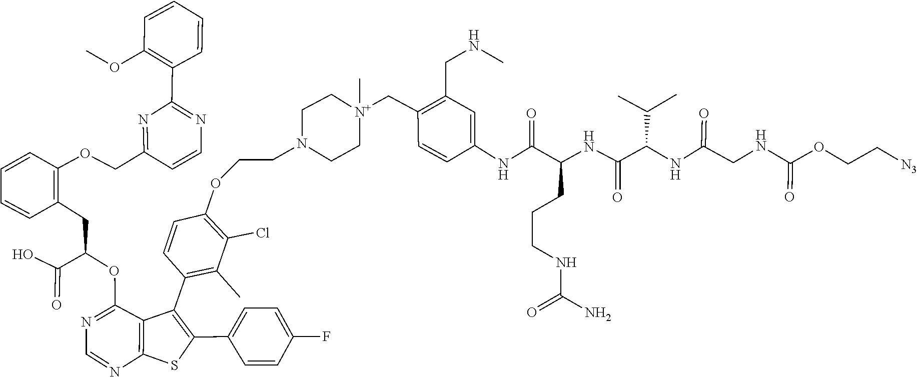 [SMILES: CNCc1cc(NC(=O)[C@H](CCCNC(N)=O)NC(=O)[C@@H](NC(=O)CNC(=O)OCCN=[N+]=[N-])C(C)C)ccc1C[N+]1(C)CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1]

To a solution of 1-(2-(((((9H-fluoren-9-yl)methoxy)carbonyl)(methyl)amino)methyl)-4-((9S,12S)-1-azido-9-isopropyl-4,7,10-trioxo-12-(3-ureidopropyl)-3-oxa-5,8,11-triazatridecan-13-amido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (112 mg, 63 μmol) in THF (3 mL) was added 2M Me₂NH in THF (627 μL, 1253 μmol). After stirring for 2 hours, the volatiles were removed in vacuo, the residue was dissolved in MeCN/H₂O and upon lyophilization 1-(4-((9S,12S)-1-azido-9-isopropyl-4,7,10-trioxo-12-(3-ureidopropyl)-3-oxa-5,8,11-triazatridecan-13-amido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (97 mg) was obtained. The material was used directly. HRMS: M⁺=1449.5800, Rt=2.14 min (5 min acidic method).

### Synthesis of 1-(2-(((((9H-fluoren-9-yl)methoxy)carbonyl)(methyl)amino)methyl)-4-((10S,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

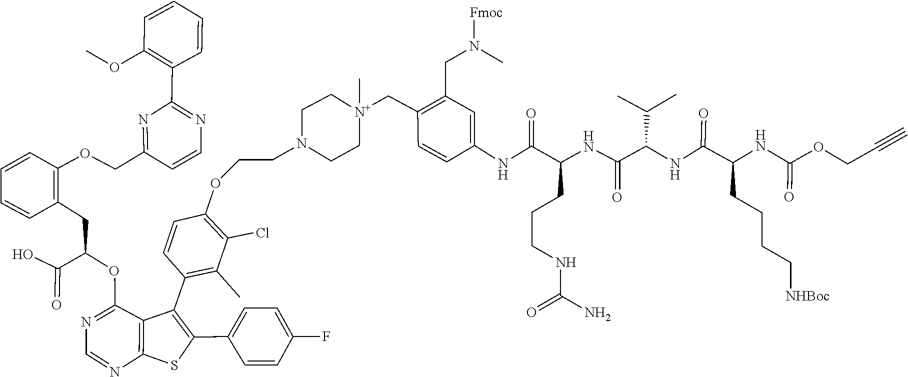

To a solution of N6-(tert-butoxycarbonyl)-N2-((prop-2-yn-1-yloxy)carbonyl)-L-lysine (44.8 mg, 137 μmol) and HATU (47.6 mg, 125 μmol) in DMF (3.5 mL) was added DIEA (79 p L, 455 μmol). After stirring for 5 minutes, 1-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium (210 mg, 114 μmol) was added. After stirring for an additional one hour, the solution was diluted with DMSO and purified by ISCO RP-HPLC. Upon lyophilization, 1-(2-(((((9H-fluoren-9-yl)methoxy)carbonyl)(methyl)amino)methyl)-4-((1OS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (124 mg, 60% yield) was obtained. HRMS: M⁺=1811.7400, Rt=2.92 min (5 min acidic method).

### Synthesis of 1-(4-((S)-2-((S)-2-((tert-butoxycarbonyl)amino)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

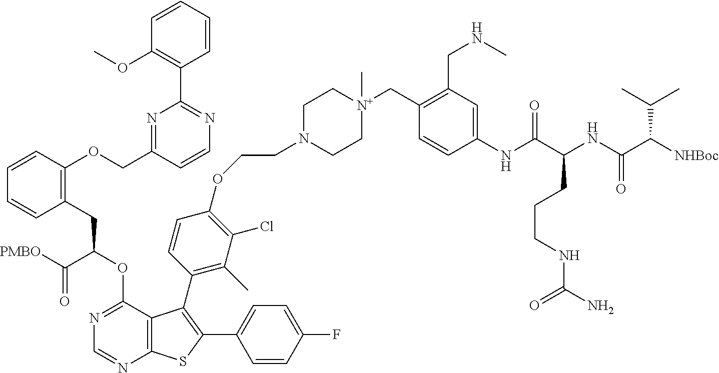

A solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (500 mg, 502 μmol, prepared based on methods described in WO2017/125224), (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate (498 mg, 653 μmol), tetrabutylammonium iodide (232 mg, 628 μmol) and DIEA (97 mg, 753 μmol) in DMF (4 mL) was stirred at rt for 20 hours at which time 2.0 M dimethyl amine in THF (3 mL, 6000 μmol) was added. After stirring for 30 minutes, the volatiles were removed in vacuo, the remaining solution was diluted with DMSO and was purified by ISCO RP-HPLC. Upon lyophilization, 1-(4-((S)-2-((S)-2-((tert-butoxycarbonyl)amino)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (700 mg, 78%) was obtained. HRMS: M⁺=1499.6400, Rt=2.36 min (5 min acidic method).

### Synthesis of 1-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium_trifluoroacetate

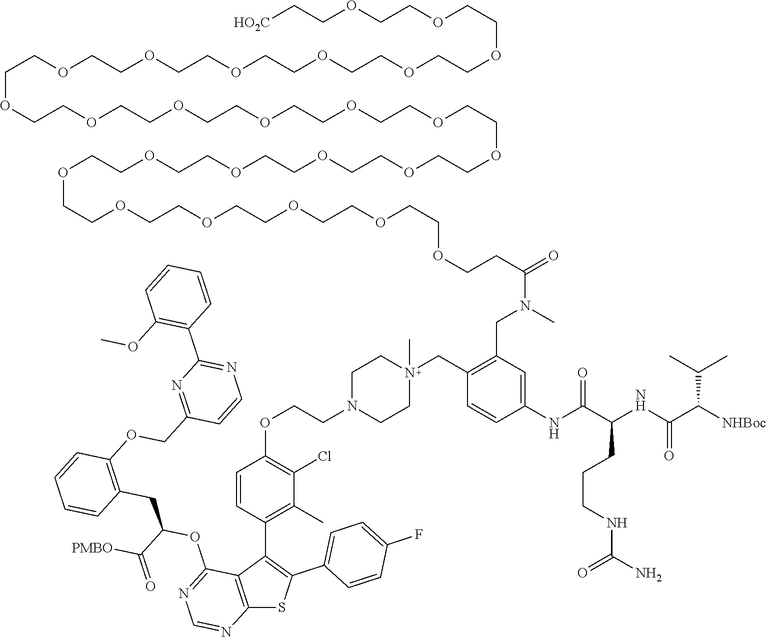

To a solution of 1-(4-((S)-2-((S)-2-((tert-butoxycarbonyl)amino)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (600 mg, 371 μmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (587 mg, 446 μmol) in DMF (3 mL) was added DIEA (324 μL, 1857 μmol). After stirring 16 hours, the solution was diluted with DMSO and purified by ISCO RP-HPLC. Upon lyophilization, 1-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (640 mg, 59% yield) was obtained. HRMS: M⁺=2700.3000, Rt=2.68 min (5 min acidic method).

### Synthesis of 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

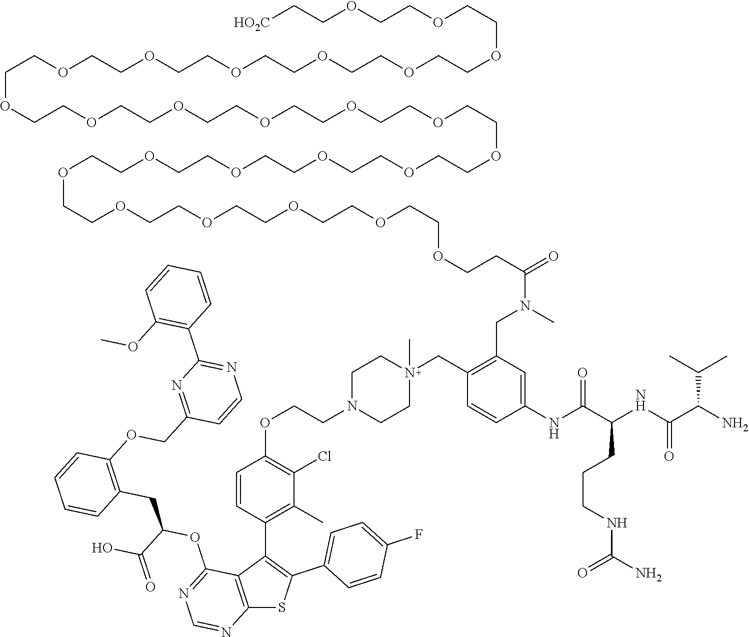

1-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (520 mg, 192 μmol) was treated with 33% TFA/CH₂Cl₂ (4.5 mL) for 1 hour at which the volatiles were removed in vacuo and the residue was triturated with diethyl ether (3×10 mL). After pumping on high vac, 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (525 mg, 93% yield) was obtained. HRMS: M⁺=2480.1899, Rt=2.12 min (5 min acidic method).

### Synthesis of 1-(2-(((((9H-fluoren-9-yl)methoxy)carbonyl)(methyl)amino)methyl)-4-((S)-2-((S)-2-(2-azidoacetamido)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

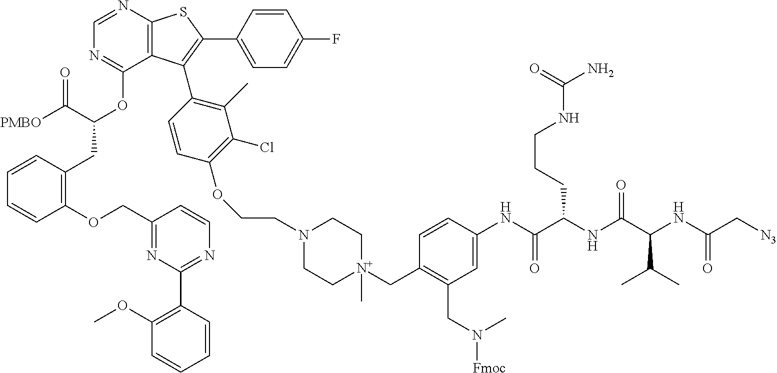

A solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (231 mg, 232 μmol, prepared based on methods described in WO2017/125224), (9H-fluoren-9-yl)methyl (5-((S)-2-((S)-2-(2-azidoacetamido)-3-methylbutanamido)-5-ureidopentanamido)-2-(chloromethyl)benzyl)(methyl)carbamate (225 mg, 302 μmol), tetrabutylammonium iodide (94 mg, 255 μmol) and DIEA (122 p L, 696 μmol) in DMSO (2 mL) was stirred at rt for 2 hours at which time the solution was diluted with DMSO and was purified by ISCO RP-HPLC. Upon lyophilization, 1-(2-(((((9H-fluoren-9-yl)methoxy)carbonyl)(methyl)amino)methyl)-4-((S)-2-((S)-2-(2-azidoacetamido)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (275 mg, 69%) was obtained. HRMS: M⁺=1704.6801, Rt=2.96 min (5 min acidic method).

### Synthesis of 1-(4-((S)-2-((S)-2-(2-azidoacetamido)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

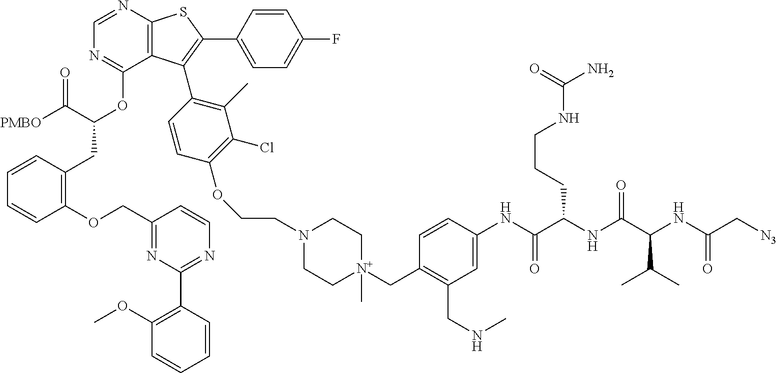

1-(2-(((((9H-fluoren-9-yl)methoxy)carbonyl)(methyl)amino)methyl)-4-((S)-2-((S)-2-(2-azidoacetamido)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (275 mg, 161 μmol) was treated with 2.0 M dimethyl amine in MeOH (1.62 mL, 3.24 mmol). After stirring for 30 minutes, the volatiles were removed in vacuo, the residue was dissolved in MeOH and purified by ISCO RP-HPLC. Upon lyophilization, 1-(4-((S)-2-((S)-2-(2-azidoacetamido)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (114 mg, 48% yield) was obtained. HRMS: M⁺=1482.5900, Rt=2.42 min (5 min acidic method).

### Synthesis of 1-(4-((S)-2-((S)-2-(2-azidoacetamido)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

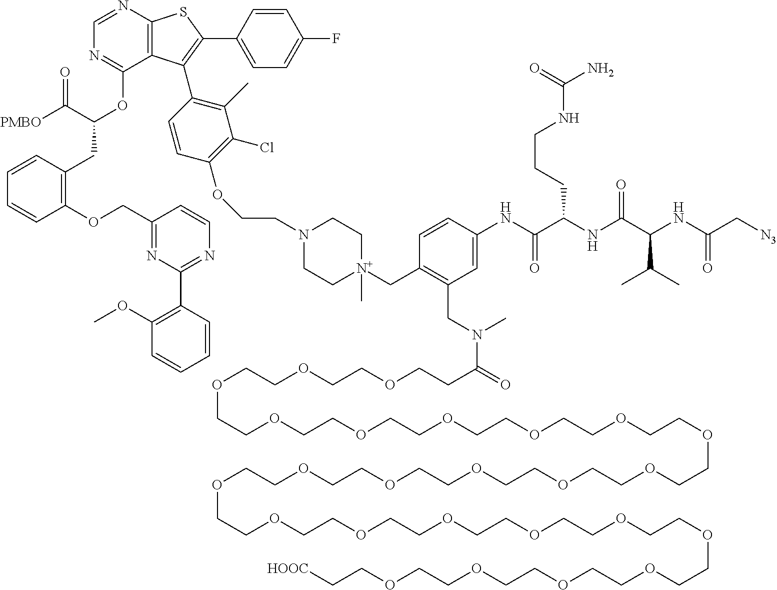

To a solution of 1-(4-((S)-2-((S)-2-(2-azidoacetamido)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (114 mg, 77 μmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (203 mg, 154 μmol) in DMF (3 mL) was added DIEA (162 μL, 926 μmol). After stirring 4 hours, the solution was diluted with DMSO and purified by ISCO RP-HPLC. Upon lyophilization, 1-(4-((S)-2-((S)-2-(2-azidoacetamido)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (89 mg, 44% yield) was obtained. HRMS: M⁺=2683.2600, Rt=2.71 min (5 min acidic method).

### Synthesis of 1-(4-((S)-2-((S)-2-(2-azidoacetamido)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

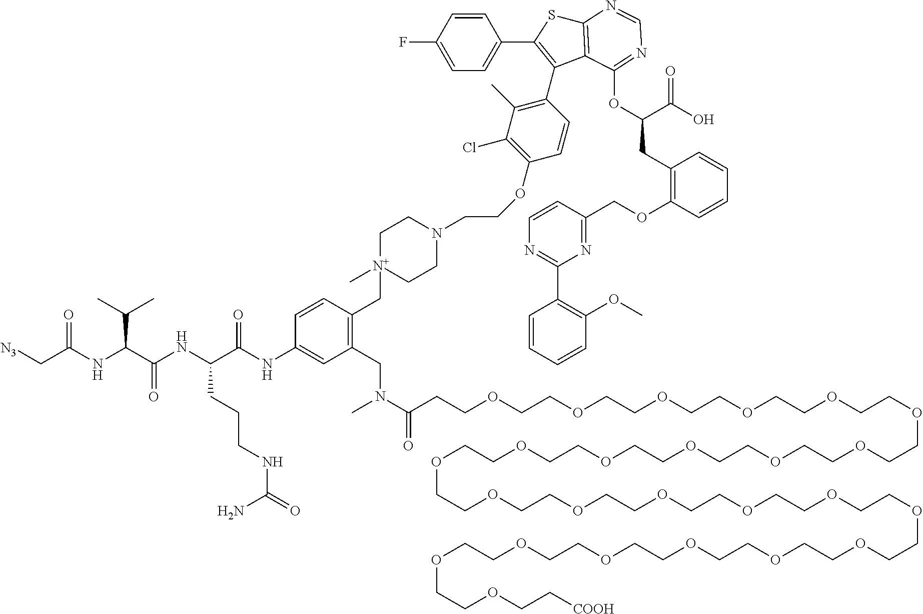 [SMILES: COc1ccccc1-c1nccc(COc2ccccc2C[C@@H](Oc2ncnc3sc(-c4ccc(F)cc4)c(-c4ccc(OCCN5CC[N+](C)(Cc6ccc(NC(=O)[C@H](CCCNC(N)=O)NC(=O)[C@@H](NC(=O)CN=[N+]=[N-])C(C)C)cc6CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)CC5)c(Cl)c4C)c23)C(=O)O)n1]

1-(4-((S)-2-((S)-2-(2-azidoacetamido)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (89 mg, 32 μmol) was treated with 25% TFA/CH₂Cl₂ with 0.1% triethyl silane for 1 hour at which time the volatiles were removed in vacuo, the residue was dissolved in DMSO and was purified by ISCO RP-HPLC. Upon lyophilization, 1-(4-((S)-2-((S)-2-(2-azidoacetamido)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (54 mg, 64% yield) was obtained. HRMS: M⁺=2563.2100, Rt=2.45 min (5 min acidic method).

### Synthesis of 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

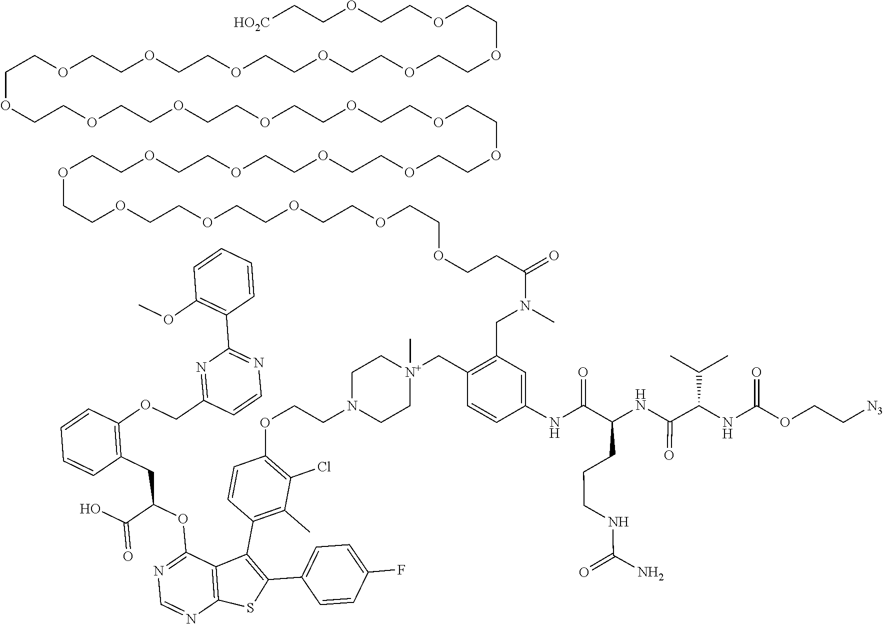 [SMILES: COc1ccccc1-c1nccc(COc2ccccc2C[C@@H](Oc2ncnc3sc(-c4ccc(F)cc4)c(-c4ccc(OCCN5CC[N+](C)(Cc6ccc(NC(=O)[C@H](CCCNC(N)=O)NC(=O)[C@@H](NC(=O)OCCN=[N+]=[N-])C(C)C)cc6CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)CC5)c(Cl)c4C)c23)C(=O)O)n1]

To a solution of 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (65 mg, 26 μmol) and 2-azidoethyl (4-nitrophenyl) carbonate (13.2 mg, 52 μmol) in DMF (0.6 mL) was added DIEA (100 μL, 210 μmol). After stirring for 16 hours, the solution was diluted with DMSO and purified by ISCO RP-HPLC. Upon lyophilization, 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (45 mg, 60% yield) was obtained. HRMS: M⁺=2593.2200, Rt=2.41 min (5 min acidic method).

### Synthesis of 1-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

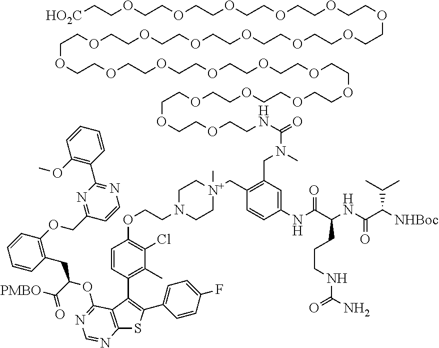

Following General Procedure #1 using bis(4-nitrophenyl) carbonate (91 mg, 300 μmol), DIEA (90 μL, 515 μmol), 1-amino-3,6,9,12,15,18,21,24,27,30,33,36,39,42,45,48,51,54,57,60,63,66,69,72-tetracosaoxapentaheptacontan-75-oic acid (355 mg, 310 μmol) and then 1-(4-((S)-2-((S)-2-((tert-butoxycarbonyl)amino)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (300 mg, 200 μmol), 1-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (444 mg, 67% yield) was obtained. HRMS: (M⁺+H⁺)+ 2/2=1366.6500, Rt=2.63 min (5 min acidic method).

### Synthesis of 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

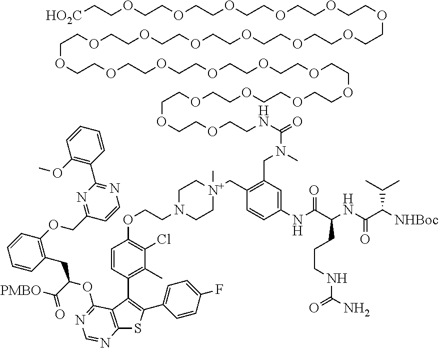

1-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (220 mg, 79 μmol) was treated with 33% TFA/CH₂Cl₂ (3 mL) for 2 hours at which time the volatiles were removed in vacuo, the residue was triturated with diethyl ether and after drying 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (230 mg) was obtained. The TFA salt was used directly. HRMS: (M⁺+H⁺)+ 2/2=1226.1000, Rt=2.08 min (5 min acidic method).

### Synthesis of 1-(4-((S)-2-((S)-2-(((2-azidoethoxy)carbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

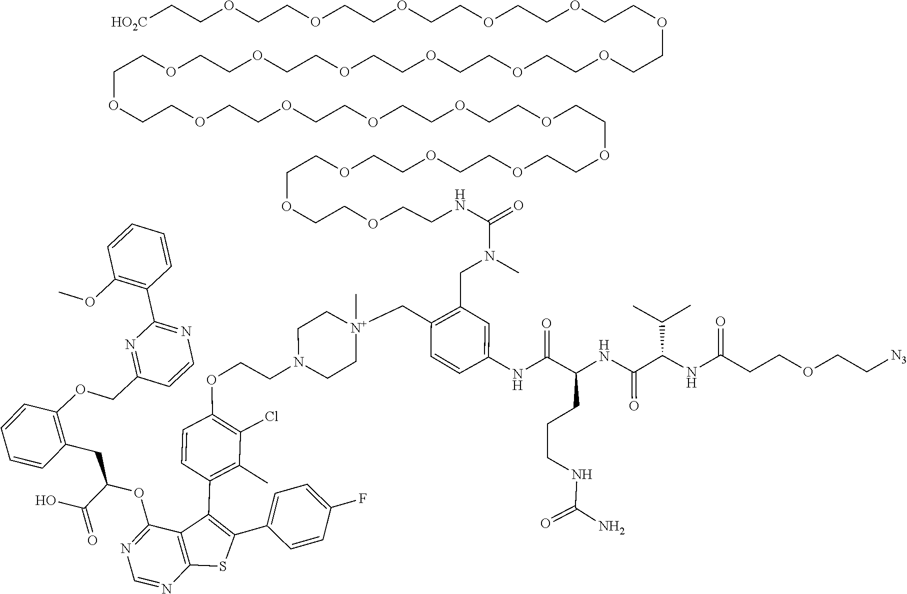 [SMILES: COc1ccccc1-c1nccc(COc2ccccc2C[C@@H](Oc2ncnc3sc(-c4ccc(F)cc4)c(-c4ccc(OCCN5CC[N+](C)(Cc6ccc(NC(=O)[C@H](CCCNC(N)=O)NC(=O)[C@@H](NC(=O)CCOCCN=[N+]=[N-])C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC5)c(Cl)c4C)c23)C(=O)O)n1]

To a solution of 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (115 mg, 43 μmol) and 2-azidoethyl (4-nitrophenyl) carbonate (18 mg, 71 μmol) in DMF (1 mL) was added DIEA (100 P L, 573 μmol). After stirring for 16 hours, the solution was diluted with DMSO and purified by ISCO RP-HPLC. Upon lyophilization, 1-(4-((S)-2-((S)-2-(((2-azidoethoxy)carbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (53 mg, 46% yield) was obtained. HRMS: M⁺=2564.2100, Rt=2.39 min (5 min acidic method).

### Synthesis of 1-(2-(((((9H-fluoren-9-yl)methoxy)carbonyl)(methyl)amino)methyl)-4-((S)-2-((S)-2-(((2-azidoethoxy)carbonyl)amino)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

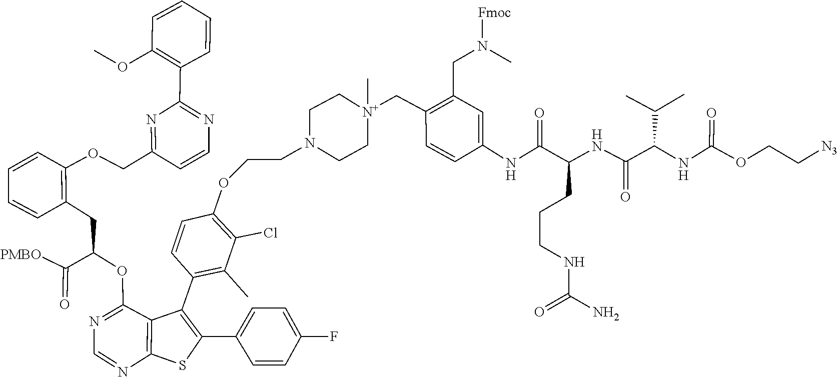

To a solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (1.3 g, 1.3 mmol, prepared based on methods described in WO2017/125224), (9H-fluoren-9-yl)methyl (5-((S)-2-((S)-2-(((2-azidoethoxy)carbonyl)amino)-3-methylbutanamido)-5-ureidopentanamido)-2-(chloromethyl)benzyl)(methyl)carbamate (1.32 g, 1.7 mmol) and tetrabutylammonium iodide (531 mg, 1.4 mmol) in DMSO (10 mL) was added DIEA (684 μL, 3.9 mmol). After stirring for 1 hour, the solution was diluted with DMSO and was purified by ISCO RP-HPLC. Upon lyophilization, 1-(2-(((((9H-fluoren-9-yl)methoxy)carbonyl)(methyl)amino)methyl)-4-((S)-2-((S)-2-(((2-azidoethoxy)carbonyl)amino)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (1.80 grams) was obtained. HRMS: M⁺=1734.6801, Rt=2.81 min (5 min acidic method).

### Synthesis of 1-(4-((S)-2-((S)-2-(((2-azidoethoxy)carbonyl)amino)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

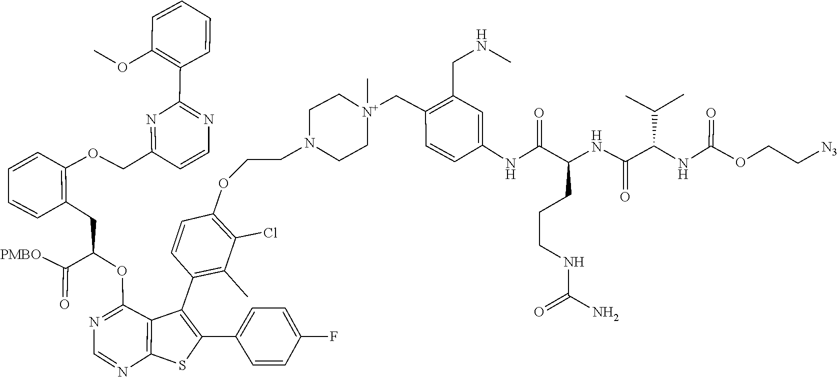

A solution of 1-(2-(((((9H-fluoren-9-yl)methoxy)carbonyl)(methyl)amino)methyl)-4-((S)-2-((S)-2-(((2-azidoethoxy)carbonyl)amino)-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (761 mg, 439 μmol) in 2.0 M dimethyl amine in MeOH (2.2 mL, 4400 μmol) was stirred for one hour at which time the volatiles were removed in vacuo, the residue was dissolved in MeOH and purified by RP-HPLC. Upon lyophilization, 1-(4-((S)-2-((S)-2-(((2-azidoethoxy)carbonyl)amino)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (526 mg, 79% yield) was obtained. HRMS: M⁺=1512.6300, Rt=2.25 min (5 min acidic method).

### Synthesis of 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

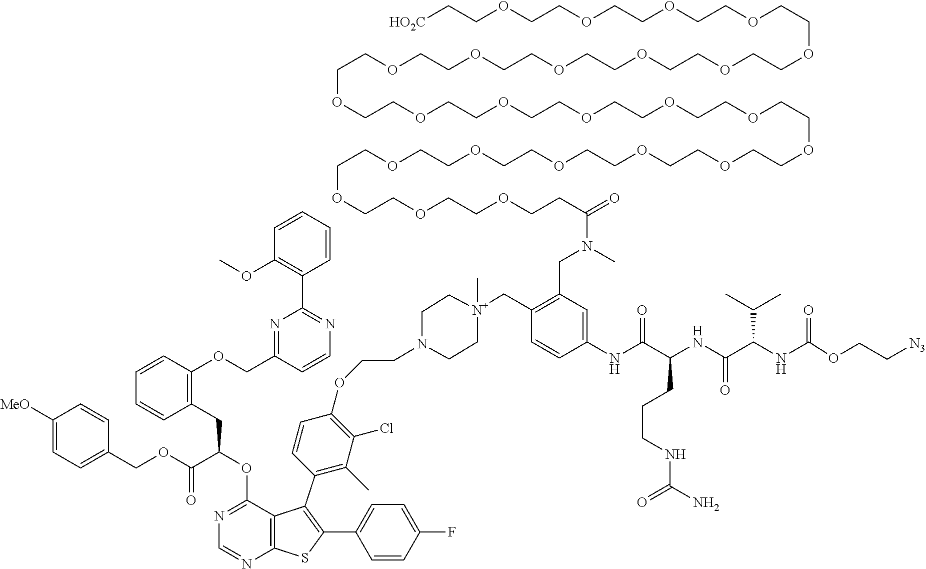 [SMILES: COc1ccc(COC(=O)[C@@H](Cc2ccccc2OCc2ccnc(-c3ccccc3OC)n2)Oc2ncnc3sc(-c4ccc(F)cc4)c(-c4ccc(OCCN5CC[N+](C)(Cc6ccc(NC(=O)[C@H](CCCNC(N)=O)NC(=O)[C@@H](NC(=O)OCCN=[N+]=[N-])C(C)C)cc6CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)CC5)c(Cl)c4C)c23)cc1]

To a solution of 1-(4-((S)-2-((S)-2-(((2-azidoethoxy)carbonyl)amino)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (150 mg, 86 μmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (125 mg, 95 μmol) in DMF (1 mL) was added DIEA (56 mg, 430 μmol). After stirring 16 hours, the solution was diluted with DMSO and purified by ISCO RP-HPLC. Upon lyophilization, 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (188 mg, 81% yield) was obtained. HRMS: M⁺=2713.2871, Rt=2.62 min (5 min acidic method).

### Synthesis of 1-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

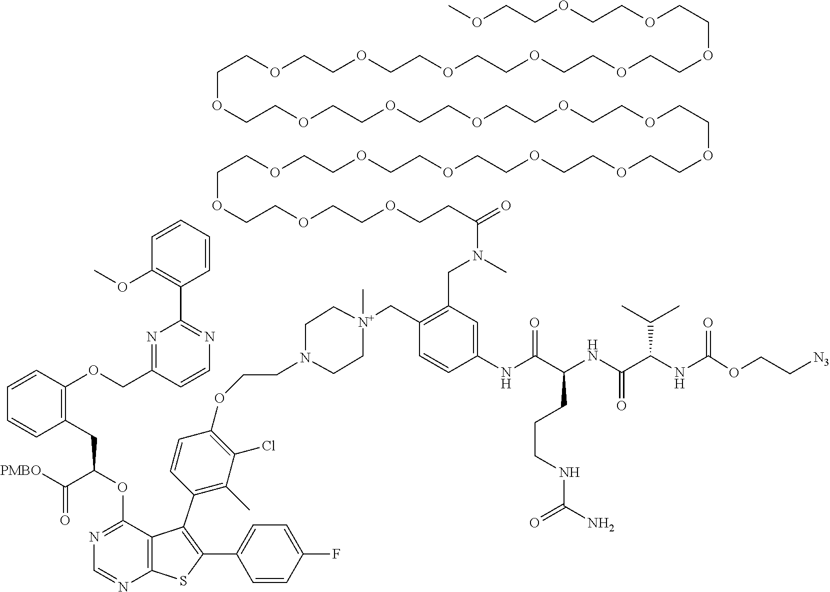

To a solution of 2,5,8,11,14,17,20,23,26,29,32,35,38,41,44,47,50,53,56,59,62,65,68,71-tetracosaoxatetraheptacontan-74-oic acid (192 mg, 172 μmol) and HATU (65.5 mg, 172 μmol) in DMF (3 mL) was added DIEA (175 μL, 1004 μmol). After stirring for 15 minutes, 1-(4-((S)-2-((S)-2-(((2-azidoethoxy)carbonyl)amino)-3-methylbutanamido)-5-ureidopentanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (250 mg, 143 μmol) was added. After stirring for an additional 30 minutes, the solution was diluted with DMSO and purified by ISCO RP-HPLC. Upon lyophilization, 1-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (110 mg, 29% yield) was obtained. HRMS: M⁺=2611.6799, Rt=2.60 min (5 min acidic method).

### Synthesis of 1-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

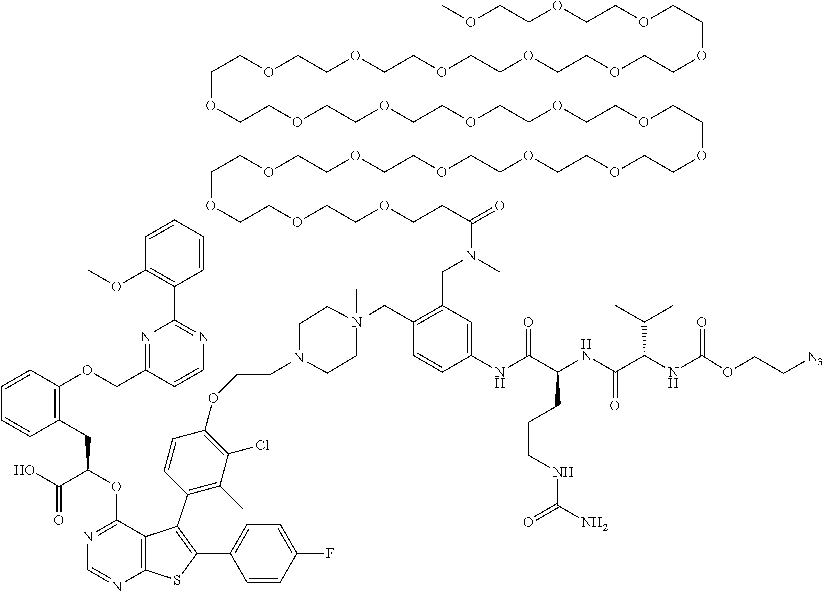 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)OCCN=[N+]=[N-])C(C)C)ccc1C[N+]1(C)CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1]

1-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (110 mg, 42 μmol) was treated with 25% TFA/CH₂Cl₂ with 0.1% triethylsilane (4 mL) for 1 hour at which time the volatiles were removed in vacuo. The residue was dissolved in DMSO and was purified by ISCO RP-HPLC. Upon lyophilization, 1-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (65 mg, 62% yield) was obtained. HRMS: M⁺=2491.1899, Rt=2.42 min (5 min acidic method).

### Synthesis of 1-(2-(((((9H-fluoren-9-yl)methoxy)carbonyl)(methyl)amino)methyl)-4-((S)-2-((S)-2-(((2-azidoethoxy)carbonyl)amino)-3-methylbutanamido)propanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

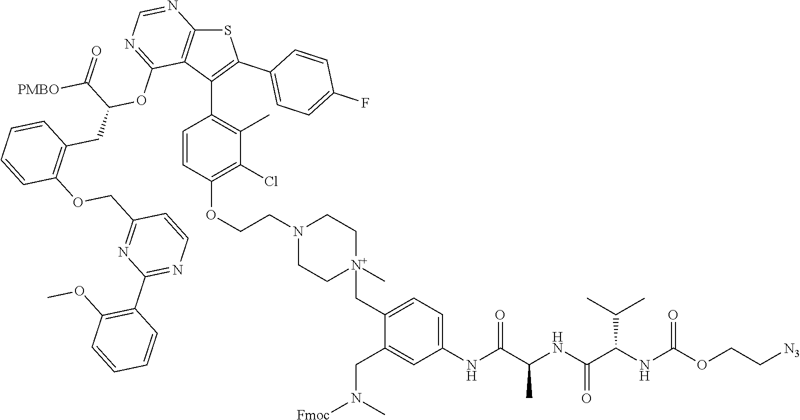

A solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (140 mg, 141 μmol, prepared based on methods described in WO2017/125224), (9H-fluoren-9-yl)methyl (5-((S)-2-((S)-2-(((2-azidoethoxy)carbonyl)amino)-3-methylbutanamido)propanamido)-2-(chloromethyl)benzyl)(methyl)carbamate (116 mg, 169 μmol), tetrabutylammonium iodide (20.8 mg, 56 μmol) and DIEA (36.4 mg, 281 μmol) in DMSO (2 mL) was stirred at rt for 1 hour at which time the solution was purified by ISCO RP-HPLC. Upon lyophilization, 1-(2-(((((9H-fluoren-9-yl)methoxy)carbonyl)(methyl)amino)methyl)-4-((S)-2-((S)-2-(((2-azidoethoxy)carbonyl)amino)-3-methylbutanamido)propanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (201 mg, 81%) was obtained. HRMS: M⁺=1648.6100, Rt=3.35 min (5 min acidic method).

### Synthesis of 1-(4-((S)-2-((S)-2-(((2-azidoethoxy)carbonyl)amino)-3-methylbutanamido)propanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

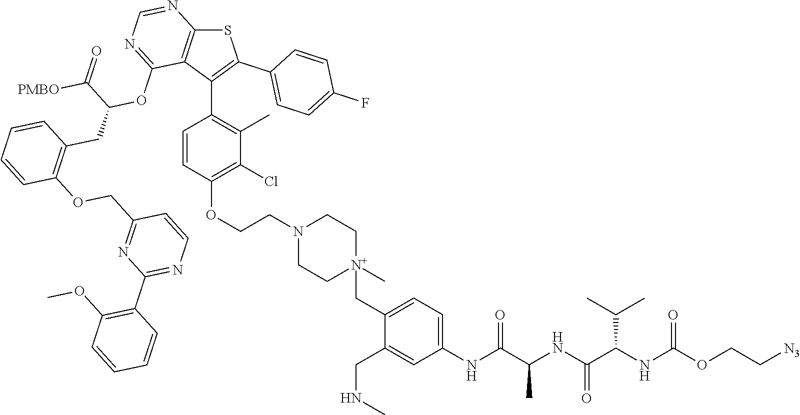

A solution of 1-(2-(((((9H-fluoren-9-yl)methoxy)carbonyl)(methyl)amino)methyl)-4-((S)-2-((S)-2-(((2-azidoethoxy)carbonyl)amino)-3-methylbutanamido)propanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (201 mg, 114 μmol) in DMSO (3 mL) was treated with 2M Me₂NH (1.14 mL, 2280 μmol). After stirring for 1 hour, the solution was purified by RP-HPLC. Upon lyophilization 1-(4-((S)-2-((S)-2-(((2-azidoethoxy)carbonyl)amino)-3-methylbutanamido)propanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (125 mg, 71% yield) was obtained. HRMS: M⁺=1426.5500, Rt=2.65 min (5 min acidic method).

### Synthesis of 1-(4-((S)-2-((S)-2-(((2-azidoethoxy)carbonyl)amino)-3-methylbutanamido)propanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

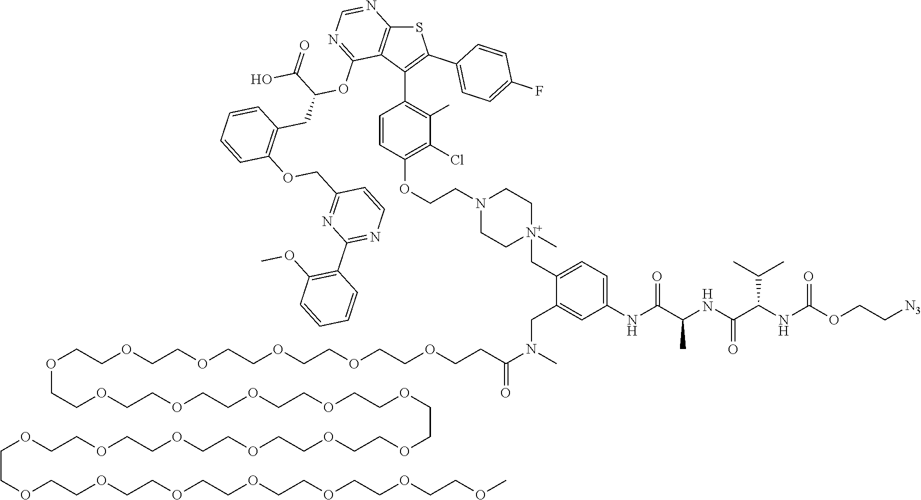 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](C)NC(=O)[C@@H](NC(=O)OCCN=[N+]=[N-])C(C)C)ccc1C[N+]1(C)CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1]

To a solution of 2,5,8,11,14,17,20,23,26,29,32,35,38,41,44,47,50,53,56,59,62,65,68,71-tetracosaoxatetraheptacontan-74-oic acid (50.9 mg, 46 μmol) and HATU (14.7 mg, 39 μmol) in DMF (2 mL) was added DIEA (37 p L, 210 μmol). After stirring for 5 minutes, 1-(4-((S)-2-((S)-2-(((2-azidoethoxy)carbonyl)amino)-3-methylbutanamido)propanamido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (62 mg, 35 μmol) was added. After stirring for an additional 30 minutes, the solution was diluted with DMSO and purified by ISCO RP-HPLC. Upon lyophilization, amide product (77.1 mg, 80% yield) was obtained. HRMS: MH⁺=2526.1899, Rt=2.96 min (5 min acidic method). The above amide (77.1 mg, 28 μmol) was treated with 25% TFA/CH₂Cl₂ (4 mL) for one hour at which time the volatiles were removed in vacuo. The residue was dissolved in MeCN/H₂O and after lyophilization 1-(4-((S)-2-((S)-2-(((2-azidoethoxy)carbonyl)amino)-3-methylbutanamido)propanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[l2,3-d]pyrimidin-5-yl)-2-chloro-3-methylphenoxy)ethyl)-1-methylpiperazin-1-ium trifluoroacetate (73.7 mg, 99% yield) was obtained. HRMS: M⁺=2405.1101, Rt=2.70 min (5 min acidic method).

### Synthesis of 1-(2-(((((9H-fluoren-9-yl)methoxy)carbonyl)(methyl)amino)methyl)-4-((10S,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

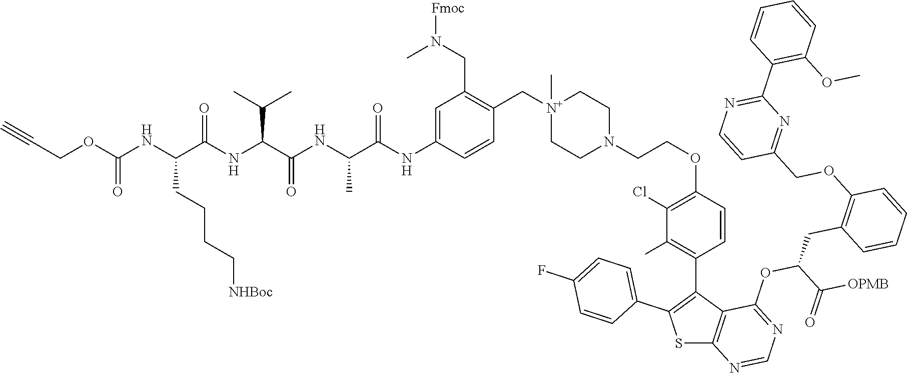

A solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (105 mg, 105 μmol, prepared based on methods described in WO2017/125224), tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxopropan-2-yl)amino)-3-methyl-1-oxobutan-2-yl)amino)-6-oxohexane-1,5-diyl)dicarbamate (112 mg, 127 μmol), tetrabutylammonium iodide (15.6 mg, 42 μmol) and DIEA (37 p L, 211 μmol) in DMSO (2 mL) was stirred at rt for 1 hour at which time the solution was purified by ISCO RP-HPLC. Upon lyophilization, 1-(2-(((((9H-fluoren-9-yl)methoxy)carbonyl)(methyl)amino)methyl)-4-((1OS,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (171 mg, 83%) was obtained. HRMS: M⁺=1845.7400, Rt=3.41 min (5 min acidic method).

### Synthesis of 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((10S,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)-2-((methylamino)methyl)benzyl)-1-methylpiperazin-1-ium trifluoroacetate

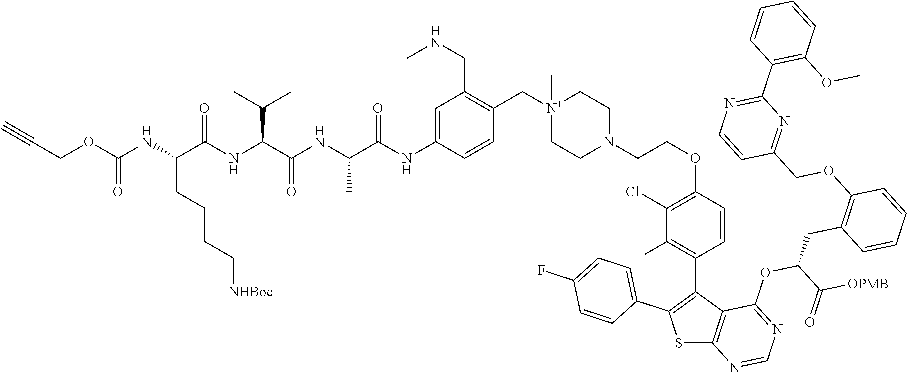

A solution of 1-(2-(((((9H-fluoren-9-yl)methoxy)carbonyl)(methyl)amino)methyl)-4-((1OS,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (171 mg, 87 μmol) in DMSO (3 mL) was treated with 2M Me₂NH in MeOH (0.872 mL, 1743 μmol). After stirring for 1 hour, the solution was purified by RP-HPLC. Upon lyophilization 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((10S,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)-2-((methylamino)methyl)benzyl)-1-methylpiperazin-1-ium trifluoroacetate (116 mg, 72% yield) was obtained. HRMS: M⁺=1623.6801, Rt=2.76 min (5 min acidic method).

### Synthesis of 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((10S,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate

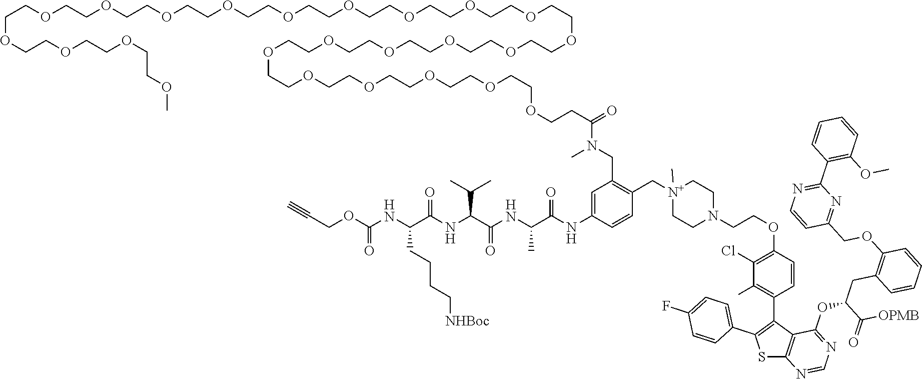

To a solution of 2,5,8,11,14,17,20,23,26,29,32,35,38,41,44,47,50,53,56,59,62,65,68,71-tetracosaoxatetraheptacontan-74-oic acid (42.8 mg, 38 μmol) and HATU (12.3 mg, 32 μmol) in DMF (2 mL) was added DIEA (31 p L, 177 μmol). After stirring for 5 minutes, 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((1OS,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)-2-((methylamino)methyl)benzyl)-1-methylpiperazin-1-ium trifluoroacetate (58 mg, 29 μmol) was added. After stirring for an additional 30 minutes, the solution was diluted with DMSO and purified by ISCO RP-HPLC. Upon lyophilization, 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((10S,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (58 mg, 66% yield) was obtained. HRMS: M⁺=2722.3999, Rt=3.04 min (5 min acidic method).

### Synthesis of 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((10S,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-((methylamino)methyl)benzyl)-1-methylpiperazin-1-ium_trifluoroacetate

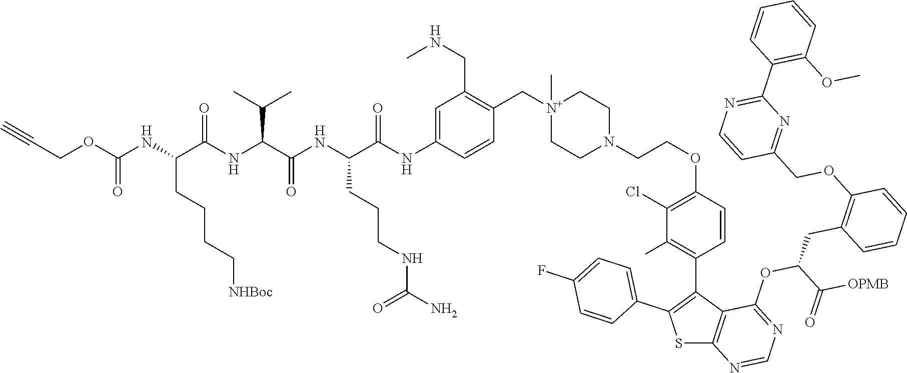

A solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (280 mg, 281 μmol, prepared based on methods described in WO2017/125224), tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxo-5-ureidopentan-2-yl)amino)-3-methyl-1-oxobutan-2-yl)amino)-6-oxohexane-1,5-diyl)dicarbamate (342 mg, 351 μmol), tetrabutylammonium iodide (114 mg, 309 μmol) and DIEA (98 mg, 562 μmol) in DMF (4.5 mL) was stirred at rt for 14 hours at which time 2.0 M dimethyl amine in THF (1.4 mL, 2800 μmol) was added. After stirring for 30 minutes, the volatiles were removed in vacuo, the remaining solution was diluted with DMSO and was purified by ISCO RP-HPLC. Upon lyophilization, 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((1OS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-((methylamino)methyl)benzyl)-1-methylpiperazin-1-ium trifluoroacetate (412 mg, 85%) was obtained. HRMS: M⁺=1709.6899, Rt=2.91 min (5 min acidic method).

### Synthesis of 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((10S,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-iumtrifluoroacetate

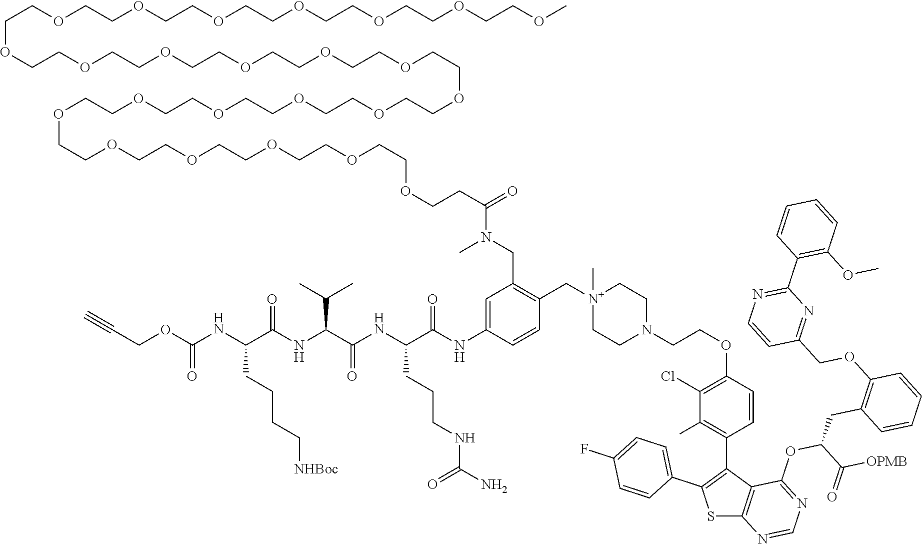

To a solution of 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((10S,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-((methylamino)methyl)benzyl)-1-methylpiperazin-1-ium trifluoroacetate (216 mg, 118 μmol) and 2,5-dioxopyrrolidin-1-yl 2,5,8,11,14,17,20,23,26,29,32,35,38,41,44,47,50,53,56,59,62,65,68,71-tetracosaoxatetraheptacontan-74-oate (180 mg, 148 μmol) in DMF was added DIEA (103 p L, 592 μmol). After stirring for 2 hours, DMSO was added and the solution was purified by RP-HPLC. After lyophilization, 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((10S,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (255 mg, 77% yield) was obtained. HRMS: M⁺=2808.3601, Rt=2.93 min (5 min acidic method).

### Synthesis of 1-(4-((2S,5S,8S)-8-(4-aminobutyl)-5-isopropyl-4,7,10-trioxo-2-(3-ureidopropyl)-11-oxa-3,6,9-triazatetradec-13-ynamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

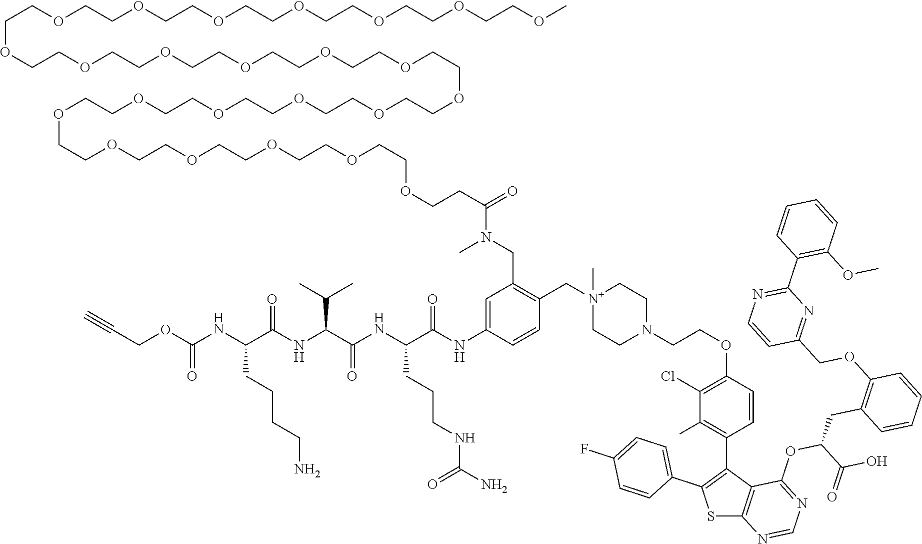 [SMILES: C#CCOC(=O)N[C@@H](CCCCN)C(=O)N[C@H](C(=O)N[C@@H](CCCNC(N)=O)C(=O)Nc1ccc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)c(CN(C)C(=O)CCOCCOCCOCCOCCOCCOCCOCCOCCOCCOCCOCCOCCOCCOCCOCCOCCOCCOCCOCCOCCOCCOCCOCCOC)c1)C(C)C]

4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((1OS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (255 mg, 91 μmol) was treated with 25% TFA/CH₂Cl₂ with 1% triethylsilane (8 mL) for 1 hour at which time the volatiles were removed in vacuo, the residue was dissolved in DMSO and purified by RP-HPLC. After lyophilization, 1-(4-((2S,5S,8S)-8-(4-aminobutyl)-5-isopropyl-4,7,10-trioxo-2-(3-ureidopropyl)-11-oxa-3,6,9-triazatetradec-13-ynamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (232 mg, 91% yield) was obtained. HRMS: M⁺=2588.2700, Rt=2.28 min (5 min acidic method).

### Synthesis of 1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((10S,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

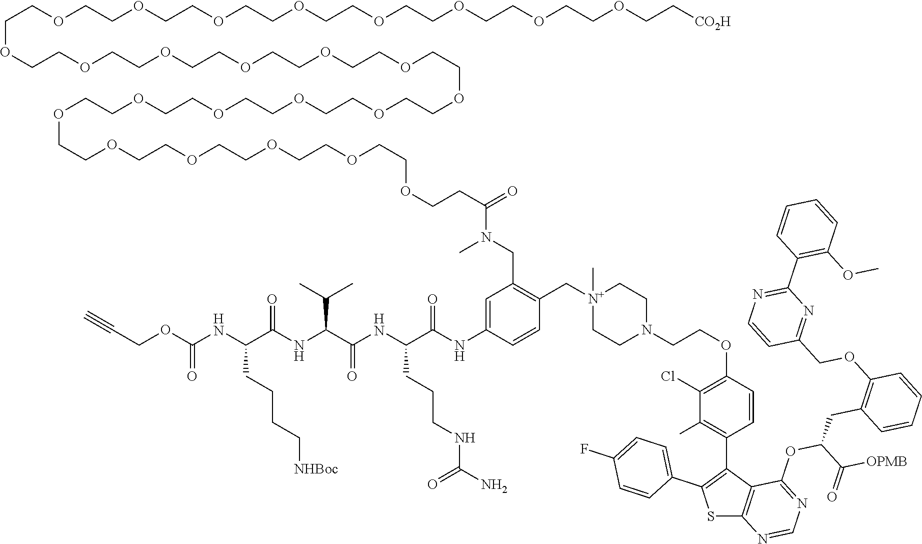

To a solution of 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((1OS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-((methylamino)methyl)benzyl)-1-methylpiperazin-1-ium trifluoroacetate (82 mg, 45 μmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (74 mg, 56 μmol) in DMF (0.8 mL) was added DIEA (39 P L, 225 μmol). After stirring for 12 hours, DMSO was added and the solution was purified by RP-HPLC. After lyophilization, 1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((10S,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (97 mg, 74% yield) was obtained. HRMS: M⁺=2910.3999, Rt=2.83 min (5 min acidic method).

### Synthesis of 1-(4-((2S,5S,8S)-8-(4-aminobutyl)-5-isopropyl-4,7,10-trioxo-2-(3-ureidopropyl)-11-oxa-3,6,9-triazatetradec-13-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

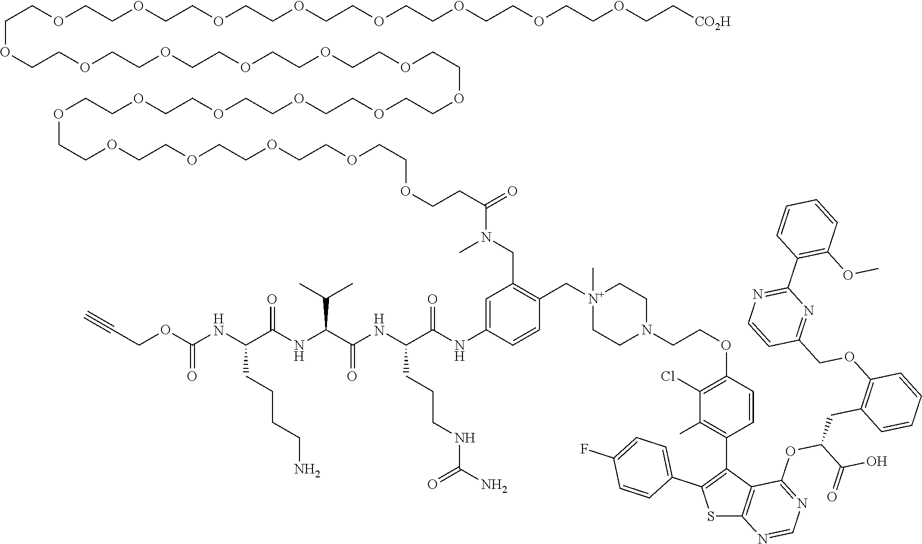 [SMILES: C#CCOC(=O)N[C@@H](CCCCN)C(=O)N[C@H](C(=O)N[C@@H](CCCNC(N)=O)C(=O)Nc1ccc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)c(CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)c1)C(C)C]

1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((1OS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (96 mg, 33 μmol) was treated with 25% TFA/CH₂Cl₂ with 1% triethylsilane (4 mL) for 1 hour at which time the volatiles were removed in vacuo, the residue was dissolved in DMSO and purified by RP-HPLC. After lyophilization, 1-(4-((2S,5S,8S)-8-(4-aminobutyl)-5-isopropyl-4,7,10-trioxo-2-(3-ureidopropyl)-11-oxa-3,6,9-triazatetradec-13-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (73 mg, 82% yield) was obtained. HRMS: M⁺=2690.2800, Rt=2.22 min (5 min acidic method).

### Synthesis of 1-(4-((S)-2-((S)-2-((tert-butoxycarbonyl)amino)-3-methylbutanamido)-5-ureidopentanamido)-2-((prop-2-yn-1-yloxy)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

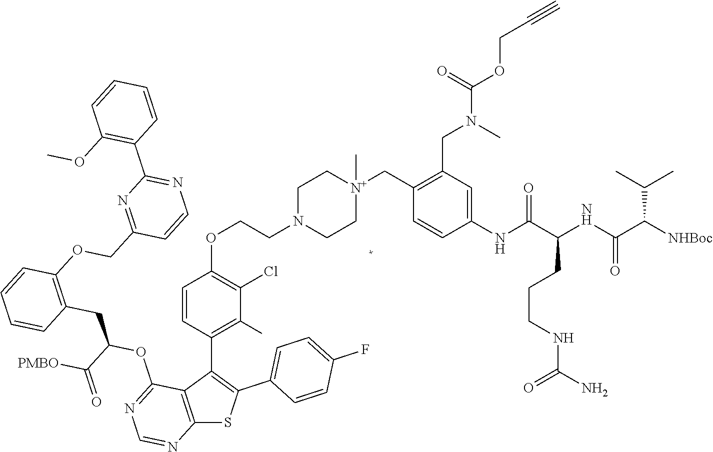

A solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (600 mg, 0.603 mmol, prepared based on methods described in WO2017/125224), tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate (0.44 g, 0.783 mmol), tetrabutylammonium iodide (267 mg, 0.723 mmol) and DIEA (840 μL, 4.82 mmol) in DMSO (5 mL) was stirred at rt for 16 hours. The solution was purified by ISCO C18 RP-HPLC. Upon lyophilization, 1-(4-((S)-2-((S)-2-((tert-butoxycarbonyl)amino)-3-methylbutanamido)-5-ureidopentanamido)-2-((prop-2-yn-1-yloxy)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (845 mg, 92%) was obtained. HRMS: M⁺=1524.6100, Rt=2.95 min (5 min acidic method).

### Synthesis of 1-(4-((S)-2-((S)-2-amino-3-methylbutanamido)-5-ureidopentanamido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

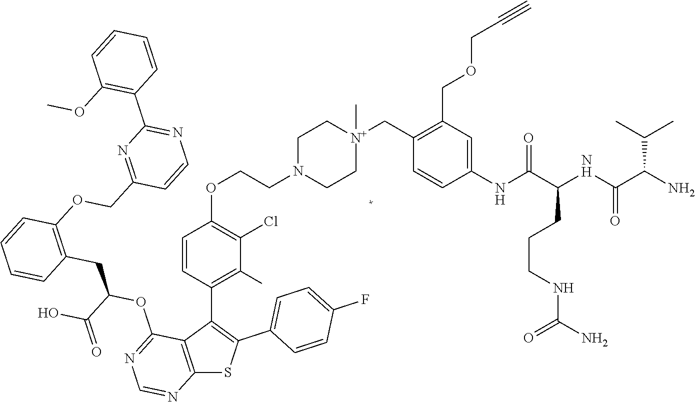 [SMILES: C#CCOCc1cc(NC(=O)[C@H](CCCNC(N)=O)NC(=O)[C@@H](N)C(C)C)ccc1C[N+]1(C)CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1]

1-(4-((S)-2-((S)-2-((tert-butoxycarbonyl)amino)-3-methylbutanamido)-5-ureidopentanamido)-2-((prop-2-yn-1-yloxy)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (845 mg, 554 μmol) was treated with 20% TFA/CH₂Cl₂ (7.5 mL) for 3 hours at which time the volatiles were removed in vacuo. The residue was dissolved in 1:1 MeCN/H₂O and lyophilized to yield 1-(4-((S)-2-((S)-2-amino-3-methylbutanamido)-5-ureidopentanamido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (915 mg). The material was used as is. HRMS: M⁺=1304.5100, Rt=2.17 min (5 min acidic method).

### Synthesis of 3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium

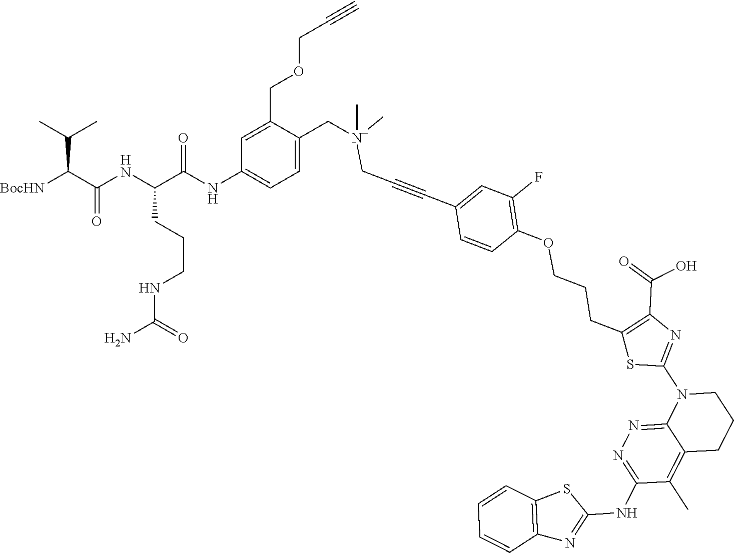 [SMILES: C#CCOCc1cc(NC(=O)[C@H](CCCNC(N)=O)NC(=O)[C@@H](NC(=O)OC(C)(C)C)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

A solution of 2-(3-(benzo[d]thiazol-2-ylamino)-4-methyl-6,7-dihydropyrido[2,3-c]pyridazin-8(5H)-yl)-5-(3-(4-(3-(dimethylamino)prop-1-yn-1-yl)-2-fluorophenoxy)propyl)thiazole-4-carboxylic acid (1.0 g, 1.52 mmol), tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate (1.03 g, 1.82 mmol), tetrabutylammonium iodide (562 mg, 1.52 mmol) and DIEA (982 mg, 7.6 mmol) in DMF (6 mL) was stirred at rt for 16 hours at which time the solution was diluted with DMSO (12 mL) and purified by ISCO C18 RP-HPLC with NH40H modifier. Upon lyophilization, 3-(4-(3-(2-(3-(benzo [d]thiazol-2-ylamino)-4-methyl-6,7-dihydropyrido [2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium trifluoroacetate (969 mg, 54%) was obtained. HRMS: M⁺=1187.4745, Rt=2.32 min (5 min acidic method).

### Synthesis of N-(4-((S)-2-((S)-2-amino-3-methylbutanamido)-5-ureidopentanamido)-2-((prop-2-yn-1-yloxy)methyl)benzyl)-3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)-N,N-dimethylprop-2-yn-1-aminium trifluoroacetate

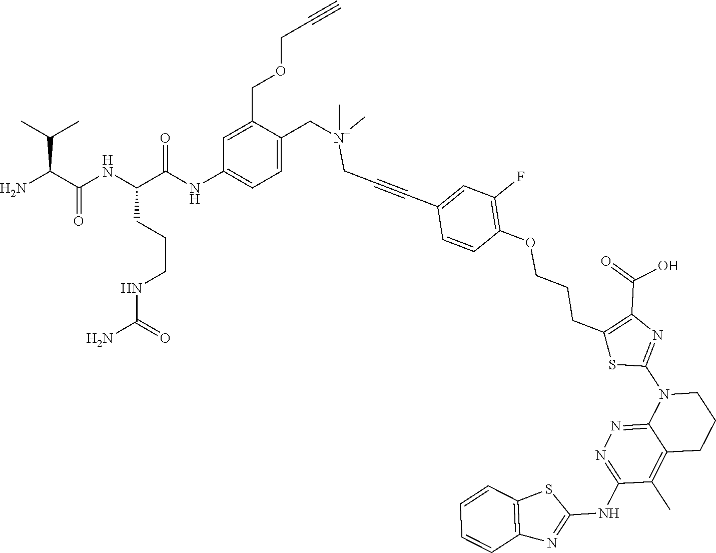 [SMILES: C#CCOCc1cc(NC(=O)[C@H](CCCNC(N)=O)NC(=O)[C@@H](N)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium (969 mg, 744 μmol) was treated with 20% TFA/CH₂Cl₂ (15 mL) for 3 hours at which time the volatiles were removed in vacuo. The residue was dissolved in 1:1 MeCN/H₂O and lyophilized to yield N-(4-((S)-2-((S)-2-amino-3-methylbutanamido)-5-ureidopentanamido)-2-((prop-2-yn-1-yloxy)methyl)benzyl)-3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)-N,N-dimethylprop-2-yn-1-aminium trifluoroacetate (1219 mg). The material was used as is. HRMS: M⁺=1087.4000, Rt=1.85 min (5 min acidic method).

### Synthesis of 4-methoxybenzyl 6-(3-(benzo[d]thiazol-2-ylamino)-4-methyl-6,7-dihydropyrido[2,3-c]pyridazin-8(5H)-yl)-3-(1-(((1r,3s,5R,7S)-3-(2-((((4-((S)-2-((S)-2-((tert-butoxycarbonyl)amino)-3-methylbutanamido)-5-ureidopentanamido)-2-((methylamino)methyl)benzyl)oxy)carbonyl)(3-hydroxypropyl)amino)ethoxy)-5,7-dimethyladamantan-1-yl)methyl)-5-methyl-1H-pyrazol-4-yl)picolinate

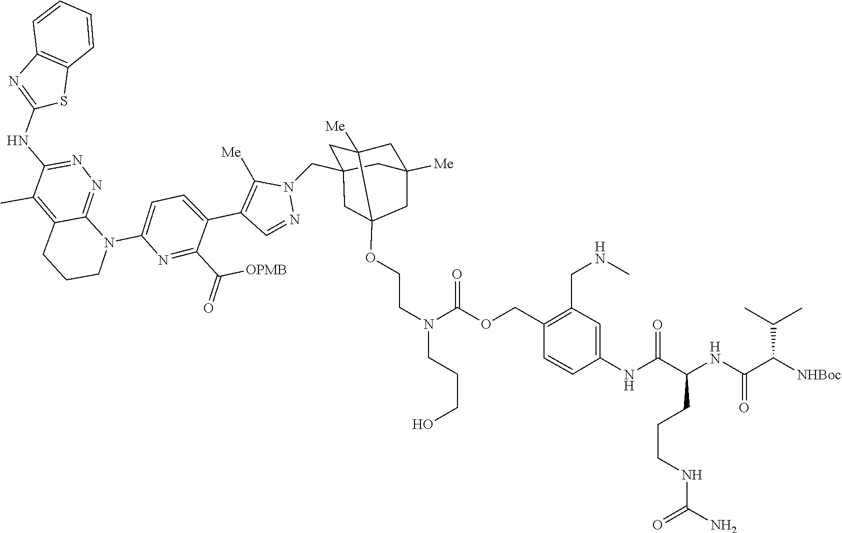

To a solution of 4-methoxybenzyl 6-(3-(benzo[d]thiazol-2-ylamino)-4-methyl-6,7-dihydropyrido[2,3-c]pyridazin-8(5H)-yl)-3-(1-(((1r,3s,5R,7S)-3-(2-((3-hydroxypropyl)amino)ethoxy)-5,7-dimethyladamantan-1-yl)methyl)-5-methyl-1H-pyrazol-4-yl)picolinate (850 mg, 856 μmol) and (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (890 mg, 978 μmol) in NMP (5 mL) was added DIEA (488 μL, 3270 μmol). After standing for 16 hours, 2.0M Me₂NH in MeOH (4.7 mL, 9.4 mmol). After standing an additional 2 hours, the solution was diluted with DMSO and purified by RP-HPLC with 0.05% formic acid modifier. After lyophilization, 4-methoxybenzyl 6-(3-(benzo[d]thiazol-2-ylamino)-4-methyl-6,7-dihydropyrido[2,3-c]pyridazin-8(5H)-yl)-3-(1-(((1r,3s,5R,7S)-3-(2-((((4-((S)-2-((S)-2-((tert-butoxycarbonyl)amino)-3-methylbutanamido)-5-ureidopentanamido)-2-((methylamino)methyl)benzyl)oxy)carbonyl)(3-hydroxypropyl)amino)ethoxy)-5,7-dimethyladamantan-1-yl)methyl)-5-methyl-1H-pyrazol-4-yl)picolinate (740 mg, 57% yield) was obtained. HRMS: MH⁺=1460.7600, Rt=2.33 min (5 min acidic method).

### Synthesis of 1-(2-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-5-((S)-2-((S)-2-((tert-butoxycarbonyl)amino)-3-methylbutanamido)-5-ureidopentanamido)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid

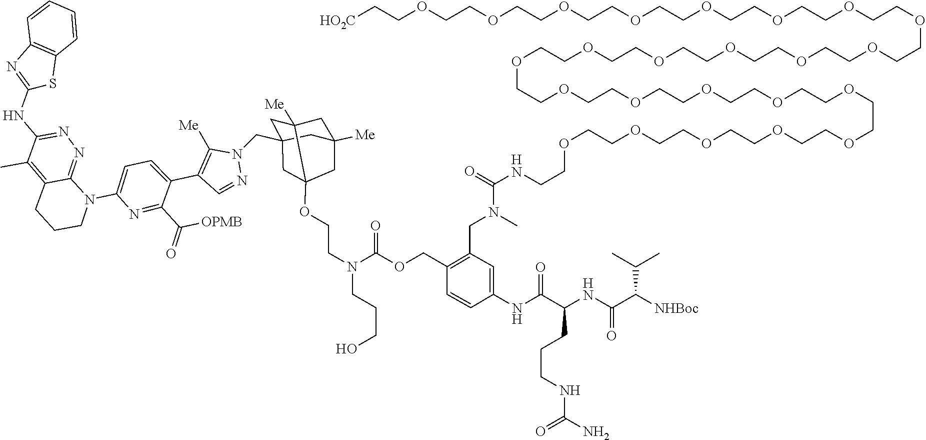

Following GENERAL PROCEDURE #1 using bis(4-nitrophenyl) carbonate (202 mg, 663 μmol), DIEA (377 μL, 2.16 μmol), 1-amino-3,6,9,12,15,18,21,24,27,30,33,36,39,42,45,48,51,54,57,60,63,66,69,72-tetracosaoxapentaheptacontan-75-oic acid (843 mg, 712 μmol) and then 4-methoxybenzyl 6-(3-(benzo[d]thiazol-2-ylamino)-4-methyl-6,7-dihydropyrido[2,3-c]pyridazin-8(5H)-yl)-3-(1-(((1r,3s,5R,7S)-3-(2-((((4-((S)-2-((S)-2-((tert-butoxycarbonyl)amino)-3-methylbutanamido)-5-ureidopentanamido)-2-((methylamino)methyl)benzyl)oxy)carbonyl)(3-hydroxypropyl)amino)ethoxy)-5,7-dimethyladamantan-1-yl)methyl)-5-methyl-1H-pyrazol-4-yl)picolinate (740 mg, 491 μmol) and additional DIEA (377 p L, 2.16 mmol), 1-(2-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-5-((S)-2-((S)-2-((tert-butoxycarbonyl)amino)-3-methylbutanamido)-5-ureidopentanamido)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (823 mg, 63%) was obtained. HRMS: MH⁺=2632.4500, Rt=2.63 min (5 min acidic method).

### Synthesis of 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid

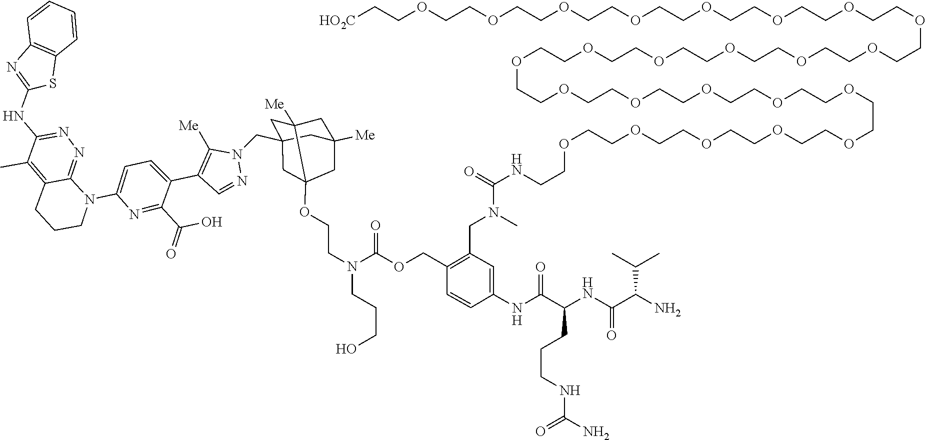 [SMILES: Cc1c(Nc2nc3ccccc3s2)nnc2c1CCCN2c1ccc(-c2cnn(CC34CC5(C)CC(C)(C3)C(OCCN(CCCO)C(=O)OCc3ccc(NC(=O)[C@H](CCCNC(N)=O)NC(=O)[C@@H](N)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

To 1-(2-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-5-((S)-2-((S)-2-((tert-butoxycarbonyl)amino)-3-methylbutanamido)-5-ureidopentanamido)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (823 mg, 312.6 μmol) was added 25% TFA/CH₂Cl₂ containing 1% Et3SiH (16 mL). After standing at rt for 1.5 hours, the volatiles were removed in vacuo, Et₂O (100 mL) was added, sonicated, settled, and decanted and material was pumped on. The residue was dissolved in MeOH (9 mL) and Me₂NH in MeOH (3 mL, ca. 20 equiv) was added to clip TFA ester present. After standing for 30 minutes, the solution was purified by RP-HPLC with 0.05% formic acid modifier. After lyophilization, 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (454 mg, 59% yield) was obtained. HRMS: MH⁺=2412.3000, Rt=2.01 min (5 min acidic method).

### Synthesis of (S)-5-(3-(tert-butoxy)-3-oxopropyl)-1-(9H-fluoren-9-yl)-3,6-dioxo-2,10,13,16,19,22-hexaoxa-4,7-diazapentacosan-25-oic acid

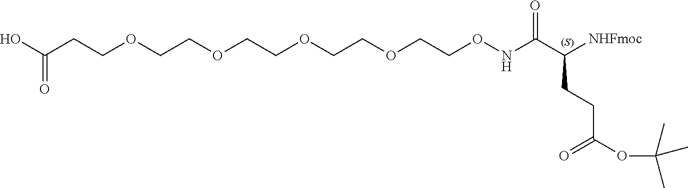

To the solution of Fmoc-Glu(OTBu)-OSu (200 mg, 0.383 mmol) in DMF(1 ml) was added the solution of Amino-PEG6-Acid (271 mg, 0.765 mmol) in MeOH(1 ml), then was followed by DIPEA (267 μl, 1.531 mmol). The resulting solution was stirred at RT for 30 min. The crude mixture was purified by C18 column (100g cartridge, MeCN/Water with 0.05% TFA 0-100% over 16CV) to obtain (S)-5-(3-(tert-butoxy)-3-oxopropyl)-1-(9H-fluoren-9-yl)-3,6-dioxo-2,10,13,16,19,22-hexaoxa-4,7-diazapentacosan-25-oic acid (273 mg, 100% yield). LC/MS: MH⁺=717.5, Rt=1.12 min (3 min acidic run).

### Synthesis of (S)-21-((((9H-fluoren-9-yl)methoxy)carbonyl)amino)-20-oxo-4,7,10,13,16-pentaoxa-19-azatetracosanedioic acid

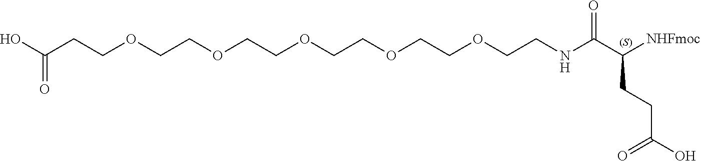

To the pre-cooled (S)-5-(3-(tert-butoxy)-3-oxopropyl)-1-(9H-fluoren-9-yl)-3,6-dioxo-2,10,13,16,19,22-hexaoxa-4,7-diazapentacosan-25-oic acid (273 mg, 0.381 mmol) and triethylsilane (0.061 mL, 0.381 mmol) was added pre-cooled TFA(25% in DCM, v/v) (11.700 mL, 38.0 mmol) at 0° C. in ice-water bath. mixture was stirred at 0° C. for 15 min, then raised to RT, and stirred at RT for 40 min. The crude mixture was concentrated under high vacuum and then dried over high vacuum overnight, and then purified by C18 column (50g cartridge, MeCN/Water with 0.05% TFA 0-100% over 16CV) to obtain (S)-21-((((9H-fluoren-9-yl)methoxy)carbonyl)amino)-20-oxo-4,7,10,13,16-pentaoxa-19-azatetracosanedioic acid (196 mg, 78% yield). LC/MS: MH⁺=661.3, Rt=0.91 min (3 min acidic run).

### Synthesis of (S)-5-(3-(tert-butoxy)-3-oxopropyl)-1-(9H-fluoren-9-yl)-3,6-dioxo-2,10,13,16,19,22,25-heptaoxa-4,7-diazaoctacosan-28-oic acid

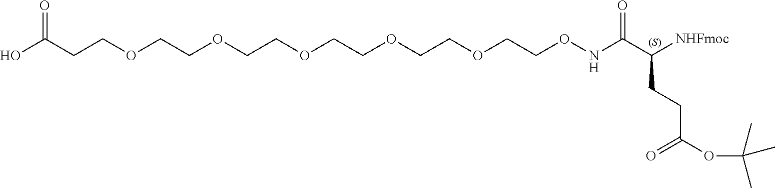

To the solution of Fmoc-Glu(OTBu)-OSu (50 mg, 0.096 mmol) in DMF(0.5 ml) was added the solution of Amino-PEG6-Acid (67.6 mg, 0.191 mmol) in MeOH(1 ml), then was followed by DIPEA (66.8 μl, 0.383 mmol). The resulting solution was stirred at RT for 30 min. The crude mixture was purified by C-18 Column(100g cartridge, MeCN/Water with 0.05% TFA 0-100% over 16CV) to obtain (S)-5-(3-(tert-butoxy)-3-oxopropyl)-1-(9H-fluoren-9-yl)-3,6-dioxo-2,10,13,16,19,22,25-heptaoxa-4,7-diazaoctacosan-28-oic acid (44 mg, 60% yield). LC/MS: MH⁺=761.5, Rt=1.12 min (3 min acidic run).

### Synthesis of (S)-24-((((9H-fluoren-9-yl)methoxy)carbonyl)amino)-23-oxo-4,7,10,13,16,19-hexaoxa-22-azaheptacosanedioic acid

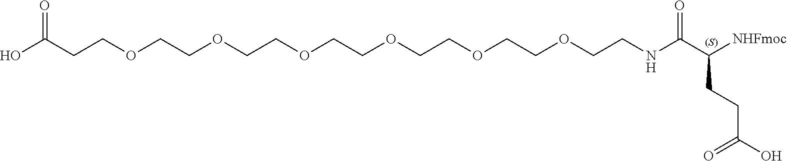

To the pre-cooled (S)-5-(3-(tert-butoxy)-3-oxopropyl)-1-(9H-fluoren-9-yl)-3,6-dioxo-2,10,13,16,19,22,25-heptaoxa-4,7-diazaoctacosan-28-oic acid (43.9 mg, 0.058 mmol) and triethylsilane (9.22 μl, 0.058 mmol) was added pre-cooled TFA(25% in DCM, v/v) (1778 μl, 5.77 mmol) at 0° C. in ice-water bath. The reaction mixture was stirred at 0° C. for 15 min, then raised to RT, and stirred at RT for 40 min. The crude mixture was concentrated under high vacuum, then dried over high vacuum overnight, and purified by C18 column (50g cartridge, MeCN/Water 0-100% with 0.05% TFA over 16CV) to obtain (S)-24-((((9H-fluoren-9-yl)methoxy)carbonyl)amino)-23-oxo-4,7,10,13,16,19-hexaoxa-22-azaheptacosanedioic acid (16 mg, 39% yield). LC/MS: MH⁺=705.4, Rt=0.91 min (3 min acidic run).

### Synthesis of di-tert-butyl (azanediylbis(ethane-2,1-diyl))bis(methylcarbamate

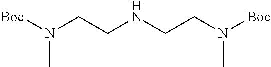 [SMILES: CN(CCNCCN(C)C(=O)OC(C)(C)C)C(=O)OC(C)(C)C]

In a 40 mL glass vial equipped with magnetic stir bar, palladium on carbon (18.32 mg, 0.172 mmol) was dissolved in EtOH (Volume: 5 mL). tert-butyl (2-aminoethyl)(methyl)carbamate (0.308 mL, 1.722 mmol) and tert-butyl methyl(2-oxoethyl)carbamate (0.298 mL, 1.722 mmol) were added. The reaction atmosphere was switched to hydrogen (treat reaction chamber with vacuum and refill with nitrogen, repeat 4×. Then treat reaction chamber with vacuum and refill with hydrogen, repeat 4×). The reaction was stirred at RT for 16 hr. The reaction was analyzed by LC-MS and the desired product was found (LC-MS crude: product ret. time: 1.45 min, [M]⁺: 332.3). The reaction atmosphere was switched to nitrogen. The reaction solution was filtered through a celite pad. The pad was then washed with EtOAc (20 mL). The combined organic layer was removed under reduced pressure. The product was then dried under high vacuum for 30 min. The product di-tert-butyl (azanediylbis(ethane-2,1-diyl))bis(methylcarbamate) was used as is in next step assuming 172 μmol.

### Synthesis of tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate

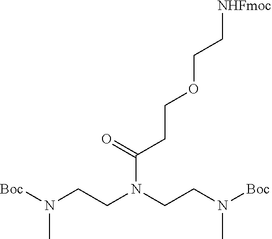

3-(2-((((9H-fluoren-9-yl)methoxy)carbonyl)amino)ethoxy)propanoic acid (367 mg, 1.034 mmol) and HATU (393 mg, 1.034 mmol) were dissolved in DMF (8 mL). DIPEA (0.542 mL, 3.10 mmol) was added. The reaction was stirred at RT for 10 min. The mixture was added to di-tert-butyl (azanediylbis(ethane-2,1-diyl))bis(methylcarbamate) (172 μmol). After stirring for 30 minutes, the reaction mixture was poured into aq. sat. ammonium chloride solution (100 mL). It was extracted with EtOAc (2×40 mL). The combined organic layer was washed with brine, treated with MgSO₄, filtered, and removed under reduced pressure. The product was then purified by SiO₂ chromatography with EtOAc/n-heptanes eluant. Upon removal of volatiles, tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate was obtained. LC/MS: MH⁺=669.3, Rt=3.03 min (5 min acidic run).

### Synthesis of tert-butyl (1-(9H-fluoren-9-yl)-11-(2-(methylamino)ethyl)-3,10-dioxo-2,7-dioxa-4,11-diazatridecan-13-yl)(methyl)carbamate trifluoroacetate

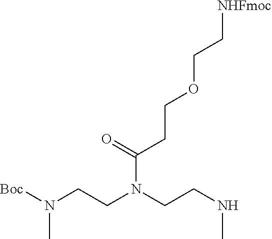

To a solution of tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate (647 mg, 967 μmol) in CH₂Cl₂ (4 mL) was added TFA (75 μL, 967 μL). After standing at rt for 14 hours, the volatiles were removed in vacuo, the residue was dissolved in DMSO and purified by ISCO RP-HPLC. Upon lyophilization, tert-butyl (1-(9H-fluoren-9-yl)-11-(2-(methylamino)ethyl)-3,10-dioxo-2,7-dioxa-4,11-diazatridecan-13-yl)(methyl)carbamate trifluoroacetate (110 mg, 20%) was obtained. LC/MS: MH⁺=569.6, Rt=0.89 min (2 min acidic run).

### Synthesis of tert-butyl 11-(2-((tert-butoxycarbonyl)(methyl)amino)ethyl)-1-(9H-fluoren-9-yl)-14-methyl-3,10,15-trioxo-2,7,18,21-tetraoxa-4,11,14-triazatetracosan-24-oate

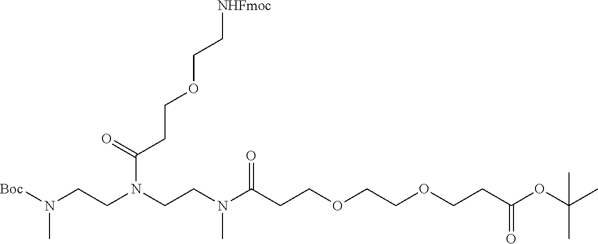

Acid-PEG2-tertbuyl ester (106 mg, 0.405 mmol) and HATU (147 mg, 0.387 mmol) were combined in DMF (2 mL). DIPEA (0.322 mL, 1.841 mmol) was added. It was left at RT for 5 min. tert-butyl (1-(9H-fluoren-9-yl)-11-(2-(methylamino)ethyl)-3,10-dioxo-2,7-dioxa-4,11-diazatridecan-13-yl)(methyl)carbamate TFA salt (251 mg, 0.368 mmol) was added. The reaction was left at RT for 5 min. The reaction was diluted with DMSO and purified by ISCO RP-HPLC. Upon lyophilization, tert-butyl 11-(2-((tert-butoxycarbonyl)(methyl)amino)ethyl)-1-(9H-fluoren-9-yl)-14-methyl-3,10,15-trioxo-2,7,18,21-tetraoxa-4,11,14-triazatetracosan-24-oate (199 mg, 67%) was obtained. LC/MS: MH⁺=813.9, Rt=1.24 min (2 min acidic run).

### Synthesis of 1-(9H-fluoren-9-yl)-14-methyl-11-(2-(methylamino)ethyl)-3,10,15-trioxo-2,7,18,21-tetraoxa-4,11,14-triazatetracosan-24-oic acid

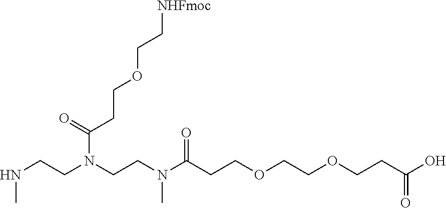

tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate (199 mg, 245 μmol) was treated with 2:1 CH₂Cl₂/TFA (3 mL) for 1 hour at which the volatiles were removed in vacuo. The residue was dissolved in DMSO and purified by ISCO RP-HPLC. Upon lyophilization, 1-(9H-fluoren-9-yl)-14-methyl-11-(2-(methylamino)ethyl)-3,10,15-trioxo-2,7,18,21-tetraoxa-4,11,14-triazatetracosan-24-oic acid (166 mg, 88% yield) was obtained as the TFA salt. LC/MS: MH⁺=656.3, Rt=0.76 min (2 min acidic run).

### Synthesis of (9H-fluoren-9-yl)methyl (2-(3-(bis(2-(methylamino)ethyl)amino)-3-oxopropoxy)ethyl)carbamate trifluoroacetate

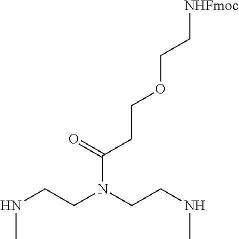

Tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate (614 mg, 918 μmol) was treated with 25% TFA/CH₂Cl₂ (8 mL) for one hour at which time the volatiles were removed in vacuo. The residue was dissolved in MeCN/H₂O and lyophilized to yield (9H-fluoren-9-yl)methyl (2-(3-(bis(2-(methylamino)ethyl)amino)-3-oxopropoxy)ethyl)carbamate trifluoroacetate. LC/MS: MH⁺=469.1, Rt=1.05 min (5 min acidic method).

### Synthesis of tert-butyl 1-(9H-fluoren-9-yl)-14-methyl-11-(2-(methylamino)ethyl)-3,10,15-trioxo-2,7,18,21-tetraoxa-4,11,14-triazatetracosan-24-oate trifluoroacetate

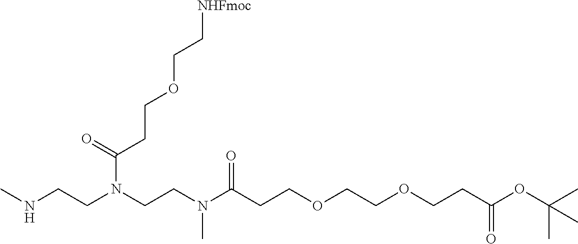

To a solution of 3-(2-(3-(tert-butoxy)-3-oxopropoxy)ethoxy)propanoic acid (40 mg, 152 μmol) and HATU (69.6 mg, 183 μmol) in DMF (2 mL) was added DIEA (160 μL, 915 μmol). After stirring for 5 minutes, (9H-fluoren-9-yl)methyl (2-(3-(bis(2-(methylamino)ethyl)amino)-3-oxopropoxy)ethyl)carbamate trifluoroacetate (425 mg, 610 μmol) was added. After stirring for an additional 30 minutes, the solution was diluted with DMSO and purified by ISCO RP-HPLC. Upon lyophilization, tert-butyl 1-(9H-fluoren-9-yl)-14-methyl-l1-(2-(methylamino)ethyl)-3,10,15-trioxo-2,7,18,21-tetraoxa-4,11,14-triazatetracosan-24-oate trifluoroacetate (26.1 mg, 21% yield) was obtained. LC/MS: MH⁺=713.6, Rt=2.09 min (5 min acidic method).

### Synthesis of di-tert-butyl 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioate

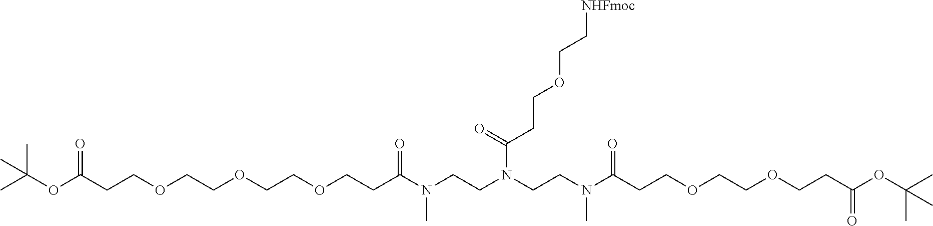

To a solution of 2,2-dimethyl-4-oxo-3,7,10,13-tetraoxahexadecan-16-oic acid (11.6 mg, 38 μmol) and HATU (12 mg, 32 μmol) in DMF (2 mL) was added DIEA (44 μL, 253 μmol). After stirring for 5 minutes, tert-butyl 1-(9H-fluoren-9-yl)-14-methyl-l1-(2-(methylamino)ethyl)-3,10,15-trioxo-2,7,18,21-tetraoxa-4,11,14-triazatetracosan-24-oate trifluoroacetate (26.1 mg, 32 μmol) was added. After stirring for an additional 30 minutes, the solution was diluted with DMSO and purified by ISCO RP-HPLC. Upon lyophilization, di-tert-butyl 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioate (21.4 mg, 68% yield) was obtained. LC/MS: MH⁺=1001.7, Rt=1.95 min (5 min acidic method).

### Synthesis of 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioic acid

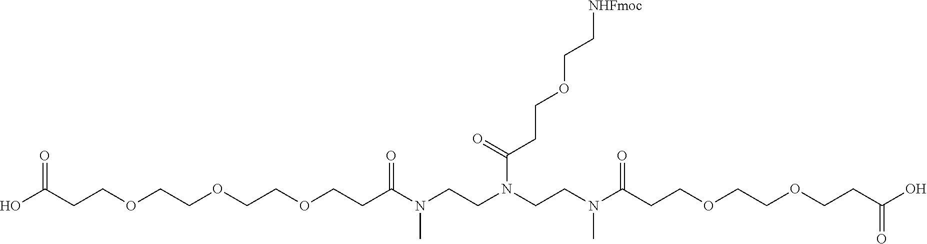

Di-tert-butyl 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioate (21.4 mg, 21 μmol) was treated with 50% TFA/CH₂Cl₂ (2 mL) for one hour at which time the volatiles were removed in vacuo. The residue was dissolved in MeCN/H₂O and lyophilized to yield 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioic acid (20.2 mg, 99% yield). LC/MS: MH⁺=889.6, Rt=1.94 min (5 min acidic method).

### Synthesis of bis(2-cyanoethyl) 16-(tert-butoxycarbonyl)-13,19-dimethyl-14,18-dioxo-4,7,10,22,25,28-hexaoxa-13,16,19-triazahentriacontanedioate

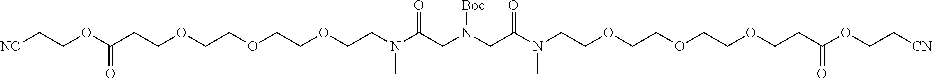 [SMILES: CN(CCOCCOCCOCCC(=O)OCCC#N)C(=O)CN(CC(=O)N(C)CCOCCOCCOCCC(=O)OCCC#N)C(=O)OC(C)(C)C]

To 2-cyanoethyl 5,8,11-trioxa-2-azatetradecan-14-oate HCl salt (3.19 grams, 8.83 mmol) and tert-butyl 2,6-dioxomorpholine-4-carboxylate (0.95 grams, 4.41 mmol) in 1:2 NMP/CH2Cl2 (15 mL) was added DIEA (2.31 mL, 13.2 mmol), HOAT (0.661 g, 4.86 mmol) and then EDC (0.931 g, 4.86 mmol). After stirring for 2 hours, the volatiles were removed in vacuo, DMSO was added and the solution was purified by RP-HPLC (with 0.05% formic acid modifier). Upon lyophilization, bis(2-cyanoethyl) 16-(tert-butoxycarbonyl)-13,19-dimethyl-14,18-dioxo-4,7,10,22,25,28-hexaoxa-13,16,19-triazahentriacontanedioate (2.10 g, 61% yield) was obtained. LC/MS: MH⁺=774.6, Rt=1.77 min (5 min acidic method).

### Synthesis of bis(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate

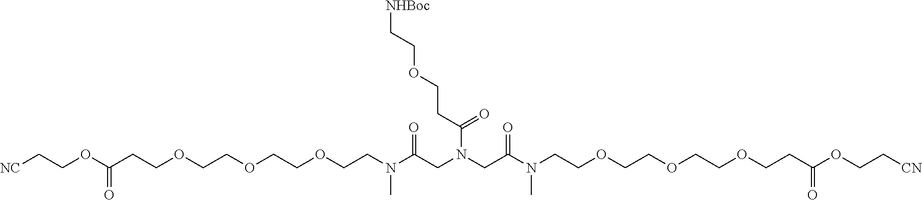 [SMILES: CN(CCOCCOCCOCCC(=O)OCCC#N)C(=O)CN(CC(=O)N(C)CCOCCOCCOCCC(=O)OCCC#N)C(=O)CCOCCNC(=O)OC(C)(C)C]

To bis(2-cyanoethyl) 16-(tert-butoxycarbonyl)-13,19-dimethyl-14,18-dioxo-4,7,10,22,25,28-hexaoxa-13,16,19-triazahentriacontanedioate (2.10 g, 2.71 mmol) was added 4 M HCl in dioxane (10 mL, 40 mmol). After standing for one hour, the volatiles were removed in vacuo. The residue was dissolved in CH₂Cl₂ (10 mL) and DIEA (1.65 mL, 9.5 mmol) was added as the solution stirred. To the neutralized solution was added HOAT (480 mg, 3.5 mmol), 3-(2-((tert-butoxycarbonyl)amino)ethoxy)propanoic acid (791 mg, 3.4 mmol) and EDC (676 mg, 3.5 mmol). After stirring for two hours, the volatiles were removed in vacuo, DMSO was added, and the solution was purified by RP-HPLC (with 0.05% formic acid modifier). Upon lyophilization, bis(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (2.30 g, 95% yield) was obtained. LC/MS: MH⁺=889.7 Synthesis of 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioic acid

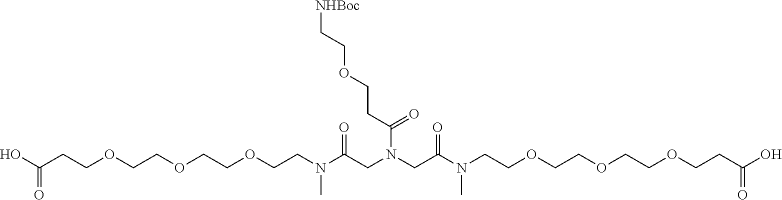 [SMILES: CN(CCOCCOCCOCCC(=O)O)C(=O)CN(CC(=O)N(C)CCOCCOCCOCCC(=O)O)C(=O)CCOCCNC(=O)OC(C)(C)C]

To bis(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (2.3 g, 2.587 mmol) in DMSO (15 mL) was added DBU (1.56 mL, 10.35 mmol). After stirring for one hour, the solution was directly purified by RP-HPLC (with 0.05% formic acid modifier). Upon lyophilization, 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioic acid (0.771 g, 38% yield) was obtained. LC/MS: MH⁺=783.6, Rt=1.50 min (5 min acidic method).

### Synthesis of bis(2,5-dioxopyrrolidin-1-yl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate

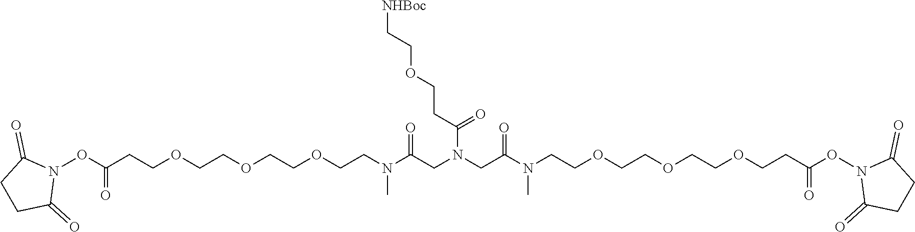 [SMILES: CN(CCOCCOCCOCCC(=O)ON1C(=O)CCC1=O)C(=O)CN(CC(=O)N(C)CCOCCOCCOCCC(=O)ON1C(=O)CCC1=O)C(=O)CCOCCNC(=O)OC(C)(C)C]

To 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioic acid (168 mg, 215 μmol) in NMP (2 mL) was added DIEA (94 p L, 537 μmol) and then TSTU (155 mg, 515 μmol). After stirring for one hour, the solution was diluted with DMSO and purified by RP-HPLC (with 0.05% formic acid modifier). Upon lyophilization, bis(2,5-dioxopyrrolidin-1-yl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (138 mg, 66% yield) was obtained. LC/MS: MH⁺=977.6, Rt=1.75 min (5 min acidic method).

### Synthesis of 16-(3-(2-((tert-butoxycarbonyl)amino)ethoxy)propanoyl)-4,7,10,13,19,22,25,28-octaoxa-16-azahentriacontanedioic acid

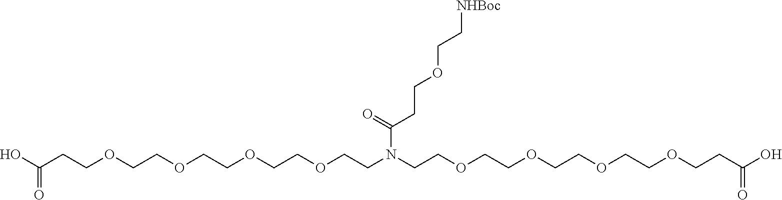 [SMILES: CC(C)(C)OC(=O)NCCOCCC(=O)N(CCOCCOCCOCCOCCC(=O)O)CCOCCOCCOCCOCCC(=O)O]

A mixture of 3-(2-((tert-butoxycarbonyl)amino)ethoxy)propanoic acid (30 mg, 0.13 mmol), HATU (42 mg, 0.11 mmol), and DIPEA (79 μL, 0.46 mmol) in DMF (1 mL) was stirred at RT for 40 min. This solution was added to 4,7,10,13,19,22,25,28-octaoxa-16-azahentriacontanedioic acid HCl salt (from BroadPharm) (50 mg, 0.091 mmol). More DIPEA (0.048 mL, 0.27 mmol) was added. The mixture was stirred at RT for 2.5 h, diluted with DMSO (1 mL) and loaded onto RP-C18 ISCO column (50g, gold). The column was eluted with MeCN-water (0.1% TFA modifier). Fractions containing the desired product were combined and lyophilized to give the product as an oil (29.7 mg, 45% yield). LC/MS: MH⁺=729.3, Rt=0.77 min (2 min acidic method).

### Synthesis of 1-(2,5-dioxopyrrolidin-1-yl) 5-methyl (tert-butoxycarbonyl)-L-glutamate

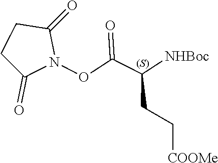

To a stirred solution of Boc-Glu(OMe)-OH (2.5 g, 9.57 mmol) and HOSu (1.211 g, 10.53 mmol) in THF(dry, 25 ml) at 0° C. in ice-water bath, was added DCC (2.073 g, 10.05 mmol) in portions. After 15 min, the reaction mixture was allowed to warm to room temperature and was stirred overnight. The solid DCU byproduct was filtered off. The filtrate was concentrated. The resulting thick oil was dissolved in DCM (40 ml), was allowed to stand for 1 h, and then was filtered to remove more DCU. The filtrate was evaporated, and the resulting glassy material was dried under high vacuum for 3h to obtain 1-(2,5-dioxopyrrolidin-1-yl) 5-methyl (tert-butoxycarbonyl)-L-glutamate (3.47 g solid foam that was carried on next reaction without further purification). LC/MS: M-Boc+1=259.2, Rt=0.81 min (3 min acidic run).

### Synthesis of (S)-6-(3-methoxy-3-oxopropyl)-2,2-dimethyl-4,7-dioxo-3,11,14,17,20,23,26-heptaoxa-5,8-diazanonacosan-29-oic acid

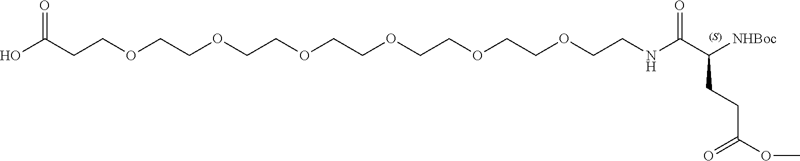

To the solution of 1-(2,5-dioxopyrrolidin-1-yl) 5-methyl (tert-butoxycarbonyl)-L-glutamate (117 mg, 0.326 mmol) in DMF(1 ml) was added the solution of Amino-PEG6-Acid (231 mg, 0.653 mmol) in MeOH(1 ml), then was followed by DIPEA (228 μl, 1.306 mmol). The resulting solution was stirred at Rt for 30 min. The crude mixture was purified by C18 column (50 g cartridge, MeCN/Water with 0.05% TFA 0-100% over 16CV) to obtain (S)-6-(3-methoxy-3-oxopropyl)-2,2-dimethyl-4,7-dioxo-3,11,14,17,20,23,26-heptaoxa-5,8-diazanonacosan-29-oic acid (138 mg, 71% yield). LC/MS MH⁺=597.5, Rt=0.75 min (3 min acidic run).

### Synthesis of ethyl 3,8-dioxo-1-phenyl-2,11,14-trioxa-4,7-diazaheptadecan-17-oate

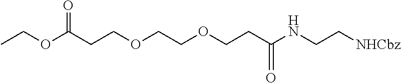 [SMILES: CCOC(=O)CCOCCOCCC(=O)NCCNC(=O)OCc1ccccc1]

A mixture of 3-(2-(3-ethoxy-3-oxopropoxy)ethoxy)propanoic acid (2.50 g, 10.7 mmol), benzyl (2-aminoethyl)carbamate (2.59 g, 11.2 mmol), HATU (4.46 g, 11.7 mmol), and DIPEA (10 mL, 57 mmol) in DMF (15 mL) was stirred at RT for 30 min. The mixture was concentrated to give the crude product, which was purified by RP-C18 ISCO column (150g, gold) by elution with MeCN-water (0.1% TFA). Fractions containing the desired product contained impurity. The fractions were combined and concentrated to remove most of MeCN. The aq. layer was basified (pH 8-9) by addition of solid K₂CO₃, and the product was extracted with EtOAc. The combined EtOAc extract was dried over Na₂SO₄, filtered, and concentrated to give the crude product as an oil. As the product was still impure, this crude product was partitioned between EtOAc and 3M aq HCl. The combined organic extract was dried over Na₂SO₄, filtered, and concentrated to give the title product in pure form as an oil (3.24 g, 74% yield). LC/MS: MH⁺=411.1, Rt=0.82 min (2 min acidic method).

### Synthesis of ethyl 2,2-dimethyl-4,12-dioxo-3,15,18-trioxa-5,8,11-triazahenicosan-21-oate

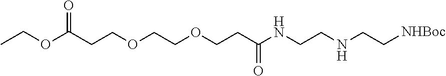 [SMILES: CCOC(=O)CCOCCOCCC(=O)NCCNCCNC(=O)OC(C)(C)C]

A mixture of ethyl 3,8-dioxo-1-phenyl-2,11,14-trioxa-4,7-diazaheptadecan-17-oate (3.24 g, 7.89 mmol), tert-butyl (2-oxoethyl)carbamate (1.257 g, 7.890 mmol), and Pd-C(10% wet) (250 mg, 2.35 mmol) in MeOH (20 mL) was hydrogenated at RT under balloon pressure for 3 days. The mixture was filtered through Celite and concentrated. The residual oil was diluted with DMSO and water. Insoluble material was removed by filtration. The filtrate was loaded onto RP-C18 ISCO column (150g, gold). The column was eluted with MeCN-water (0.1% TFA). Fractions containing the desired product were combined and concentrated to give the product (TFA salt) as an oil (910 mg, 22% yield). LC/MS: MH⁺=420.5, Rt=0.56 min (2 min acidic method).

### Synthesis of 2,2-dimethyl-4,12-dioxo-3,15,18-trioxa-5,8,11-triazahenicosan-21-oic acid

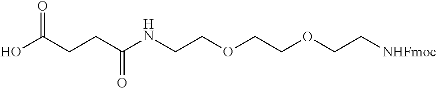

A mixture of ethyl 2,2-dimethyl-4,12-dioxo-3,15,18-trioxa-5,8,11-triazahenicosan-21-oate (910 mg, 2.17 mmol) and aq. LiOH (1M, 2.4 mL, 2.4 mmol) in MeOH (2.4 mL) and THF (2.4 mL) was heated at 50° C. for 0.5 h. LCMS analysis showed no reaction. The mixture was cooled to RT, and a solution of KOH (260 mg, 4.64 mmol) in water (1 mL) was added. The mixture was stirred at RT for 10 min. The mixture was concentrated, and the residue was taken up in water and loaded onto RP-C18 ISCO column (50g, gold). The column was eluted with MeCN-water (0.1% TFA). Fractions containing the desired product were combined and concentrated to give the product (TFA salt) as a white solid (699 mg, 64% yield). LC/MS: MH⁺=392.5, Rt=0.56 min (2 min acidic method).

### Synthesis of 8-(((9H-fluoren-9-yl)methoxy)carbonyl)-2,2-dimethyl-4,12-dioxo-3,15,18-trioxa-5,8,11-triazahenicosan-21-oic acid

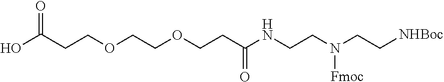

A mixture of 2,2-dimethyl-4,12-dioxo-3,15,18-trioxa-5,8,11-triazahenicosan-21-oic acid (699 mg, 1.79 mmol), (9H-fluoren-9-yl)methyl (2,5-dioxopyrrolidin-1-yl) carbonate (783 mg, 2.32 mmol), and DIPEA (1.00 mL, 5.73 mmol) in DMF (10 mL) was stirred at RT for 15 h. More (9H-fluoren-9-yl)methyl (2,5-dioxopyrrolidin-1-yl) carbonate (800 mg, 2.37 mmol) and DIPEA (0.50 mL, 2.9 mmol) were added. The mixture was stirred at RT for 1 h. The mixture was taken up in DMSO and loaded onto RP-C18 ISCO column (150g, gold). The column was eluted with MeCN-water (0.1% TFA). Fractions containing the desired product were combined and concentrated to give the product as an oil (579 mg). LCMS analysis showed partial N-de-Boc of the product. The mixture was taken up in MeCN (5 mL) and treated with Boc20 (250 mg, 1.15 mmol) and DIPEA (1.00 mL, 5.73 mmol). The mixture was stirred at RT for 40 min and partitioned between EtOAc and 0.5 M aq. HCl. The combined organic extract was dried over Na₂SO₄ and concentrated to give the crude product (743 mg). This product was directly used in the next step without purification. LC/MS: MH⁺=614.5, Rt=1.06 min (2 min acidic method).

### Synthesis of allyl 8-(((9H-fluoren-9-yl)methoxy)carbonyl)-2,2-dimethyl-4,12-dioxo-3,15,18-trioxa-5,8,11-triazahenicosan-21-oate

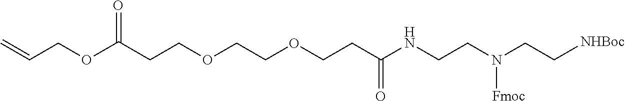

DMAP (0.012 g, 0.10 mmol) was added to a mixture of 8-(((9H-fluoren-9-yl)methoxy)carbonyl)-2,2-dimethyl-4,12-dioxo-3,15,18-trioxa-5,8,11-triazahenicosan-21-oic acid (743 mg), DCC (0.250 g, 1.21 mmol), and allyl alcohol (0.200 mL, 2.94 mmol) in THF (4 mL). The suspension was stirred at RT for 30 min (LCMS 1). The mixture was concentrated and purified by NP-ISCO (10-100% EtOAc in heptane, followed by 5% MeOH in EtOAc). Fractions containing the desired product were combined and concentrated to give a mixture of solid and oil. The mixture was triturated with ether and solid was removed by filtration. The ether layer was concentrated to give the title product as an oil (400 mg, 34% yield over the last 2 steps). LC/MS: MH⁺=654.7, Rt=1.16 min (2 min acidic method).

### Synthesis of allyl 4-(2-aminoethyl)-1-(9H-fluoren-9-yl)-3,8-dioxo-2,11,14-trioxa-4,7-diazaheptadecan-17-oate

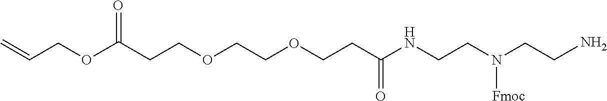

A mixture of allyl 8-(((9H-fluoren-9-yl)methoxy)carbonyl)-2,2-dimethyl-4,12-dioxo-3,15,18-trioxa-5,8,11-triazahenicosan-21-oate (400 mg, 0.612 mmol) and TFA (2 mL, 26.0 mmol) in DCM (4 mL) was stirred at RT for 20 min. The mixture was concentrated. The residue was taken up in MeCN and concentrated to remove as much TFA as possible to give the crude product, which was used in the next step without purification. LC/MS: MH⁺=554.6, Rt=0.75 min (2 min acidic method).

### Synthesis of 13,21,30-trioxo-4,7,10,24,27,31-hexaoxa-14,17,20-triazatetratriacont-33-enoic acid

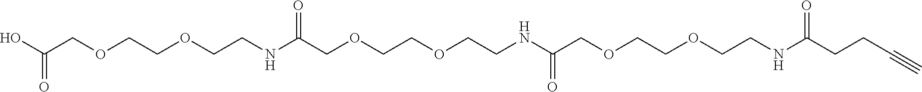 [SMILES: C#CCCC(=O)NCCOCCOCC(=O)NCCOCCOCC(=O)NCCOCCOCC(=O)O]

A mixture of allyl 4-(2-aminoethyl)-1-(9H-fluoren-9-yl)-3,8-dioxo-2,11,14-trioxa-4,7-diazaheptadecan-17-oate (crude from the previous step), 3-(2-(2-(3-((2,5-dioxopyrrolidin-1-yl)oxy)-3-oxopropoxy)ethoxy)ethoxy)propanoic acid (0.250 g, 0.720 mmol), and DIPEA (0.75 mL, 4.29 mmol) in DMF (3 mL) was stirred at RT for 40 min. Amidation was complete (LC/MS MH⁺786.8, Rt=0.96 min, 2 min acidic method). To this was then added dimethylamine in THF (2M, 3 mL, 6 mmol). The mixture was stirred at RT for 30 min. The mixture was concentrated and the residue was diluted with DMSO (5 mL), loaded onto RP-C18 ISCO column (100g, gold), and eluted with MeCN-water (0.1% TFA). Fractions containing the desired product were combined and concentrated to give the title product (TFA salt) as an oil (355 mg, 86% yield over the last 2 steps). LC/MS: MH⁺=564.6, Rt=0.52 min (2 min acidic method).

### Synthesis of 17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid

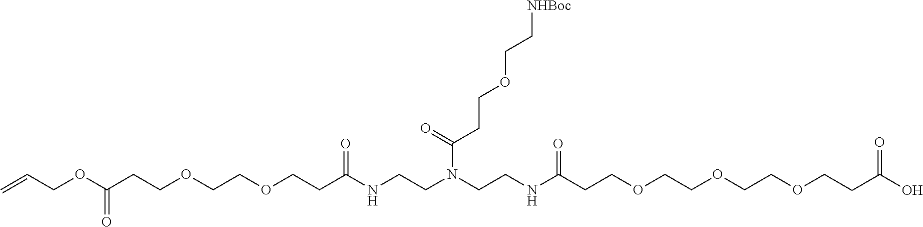 [SMILES: C=CCOC(=O)CCOCCOCCC(=O)NCCN(CCNC(=O)CCOCCOCCOCCC(=O)O)C(=O)CCOCCNC(=O)OC(C)(C)C]

A mixture of 13,21,30-trioxo-4,7,10,24,27,31-hexaoxa-14,17,20-triazatetratriacont-33-enoic acid (181 mg, 0.778 mmol), HATU (255 mg, 0.671 mmol), and DIPEA (450 μL, 2.58 mmol) in DMF (2 mL) was stirred at RT for 10 min. This was added to a DMF (1 mL) solution of 13,21,30-trioxo-4,7,10,24,27,31-hexaoxa-14,17,20-triazatetratriacont-33-enoic acid, trifluoroacetate (350 mg, 516 μmol). The mixture was stirred at RT for 10 min. The mixture was diluted with DMSO (3 mL) and loaded onto RP-ISCO column (100g, gold). The column was eluted with MeCN-water (0.1% TFA). Fractions containing the desired product were combined and concentrated by ROTOVAP to give the product as an oil (340 mg). As the product contained -15% of N-de-Boc product, the product was taken up in MeCN (2 mL) and treated with Boc20 (40 mg, 0.184 mmol) and DIPEA (100 p L, 0.573 mmol). The mixture was stirred at RT for 30 min, concentrated, and the residue was taken up in DMSO and purified by RP- C18 ISCO column (50g, gold)(MeCN-water 0.1% TFA). Fractions containing the desired product were combined and lyophilized to give the title product as an oil (236 mg, 59% yield). LC/MS: MH⁺=779.9, Rt=0.80 min (2 min acidic method).

Synthesis of tert-butyl (2-((2-((((9H-fluoren-9-yl)methoxy)carbonyl)amino)ethyl)amino)ethyl)carbamate

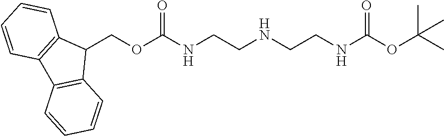 [SMILES: CC(C)(C)OC(=O)NCCNCCNC(=O)OCC1c2ccccc2-c2ccccc21]

In a 40 mL glass vial equipped with magnetic stir bar, palladium on carbon (0.067 g, 0.627 mmol), (9H-fluoren-9-yl)methyl (2-aminoethyl)carbamate (2 g, 6.27 mmol) and tert-butyl (2-oxoethyl)carbamate (0.999 g, 6.27 mmol) were dissolved in EtOH (16 mL). Acetic acid (0.036 mL, 0.627 mmol) and DIPEA (1.096 mL, 6.27 mmol) were added. The reaction atmosphere was switched to hydrogen (treat reaction chamber with vacuum and refill with nitrogen, repeat 4×. Then treat reaction chamber with vacuum and refill with hydrogen, repeat 4×). The reaction was stirred at RT for 16 hr. The reaction solution was filtered through a celite pad. The pad was then washed with EtOAc (20 mL). The combined organic layer was removed under reduced pressure. The product was then dried under high vacuum for 30 min. The product was purified by reverse phase chromatography (Diluted with 4 mL DMSO, ISCO Gold C18 Column, 150 gr, Water/MeCN as eluent, 0.1% TFA as modifier). Fractions containing the desired product were combined and lyophilized to give the title product (TFA salt)(827 mg, 24% yield). LC/MS: MH⁺=426.4, Rt=0.86 (2 min acidic method).

### Synthesis of benzyl (2-((((9H-fluoren-9-yl)methoxy)carbonyl)amino)ethyl)(2-((tert-butoxycarbonyl)amino)ethyl)carbamate

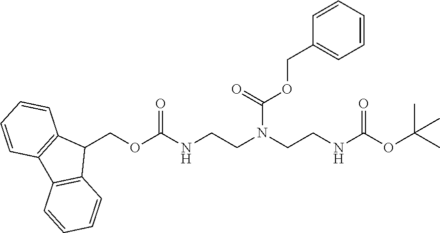 [SMILES: CC(C)(C)OC(=O)NCCN(CCNC(=O)OCC1c2ccccc2-c2ccccc21)C(=O)OCc1ccccc1]

Dissolved tert-butyl (2-((2-((((9H-fluoren-9-yl)methoxy)carbonyl)amino)ethyl)amino)ethyl)carbamate (827 mg, 1.533 mmol) in DCM (12 mL). Benzyl chloroformate (0.263 mL, 1.839 mmol) and DIPEA (0.803 mL, 4.60 mmol) were added. The reaction was stirred at RT for 1 hr. The reaction mixture was poured into aq. sat. ammonium chloride solution. The aqueous layer was extracted with EtOAc (2×). The combined organic layer was washed with brine, treated with MgSO₄, filtered, and removed under reduced pressure. The product was purified by flash chromatography (ISCO Silica column, 120 gr, MeOH/DCM as eluent, 0% to 10%) to give the title compound (859 mg, quantitative yield). LC/MS: MH⁺(-Boc) 460.5, Rt=1.26 min (2 min acidic method).

### Synthesis of benzyl (2-((((9H-fluoren-9-yl)methoxy)carbonyl)amino)ethyl)(2-aminoethyl)carbamate

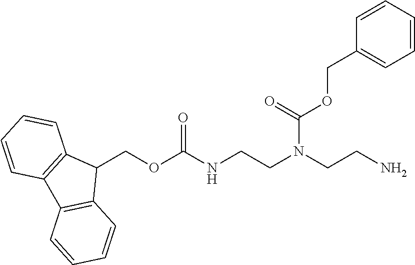 [SMILES: NCCN(CCNC(=O)OCC1c2ccccc2-c2ccccc21)C(=O)OCc1ccccc1]

In a 100 mL round bottom flask equipped with magnetic stir bar, benzyl (2-((((9H-fluoren-9-yl)methoxy)carbonyl)amino)ethyl)(2-((tert-butoxycarbonyl)amino)ethyl)carbamate (859 mg, 1.535 mmol) was dissolved in DCM (3 mL) and TFA (1 mL). The reaction was stirred at RT for 1 hr. DCE (30 mL) was added. The solvent was removed under reduced pressure to obtain the title product (TFA salt) (986 mg, 93% yield). The product was used as it is without further purification or characterization.

### Synthesis of ethyl 7-((benzyloxy)carbonyl)-1-(9H-fluoren-9-yl)-3,11-dioxo-2,14,17-trioxa-4,7,10-triazaicosan-20-oate

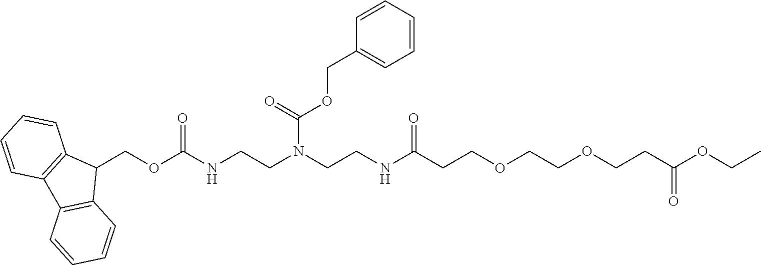 [SMILES: CCOC(=O)CCOCCOCCC(=O)NCCN(CCNC(=O)OCC1c2ccccc2-c2ccccc21)C(=O)OCc1ccccc1]

To a solution of 3-(2-(3-ethoxy-3-oxopropoxy)ethoxy)propanoic acid (230 mg, 0.982 mmol) in DMF (1.5 mL) was added HATU (299 mg, 0.785 mmol), DIPEA (0.572 mL, 3.27 mmol), and benzyl (2-((((9H-fluoren-9-yl)methoxy)carbonyl)amino)ethyl)(2-aminoethyl)carbamate (450 mg, 0.654 mmol). The mixture was stirred at RT for 1 h and concentrated. The residue was diluted with DMSO (3 mL) and purified by RP-ISCO chromatography (100 G ISCO Gold C18 Column, Water/MeCN as eluent, 0.1% TFA as modifier). The desired product fractions were combined and lyophilized to afford the title compound (206 mg, 44.2% yield). LC/MS: MH⁺=676, Rt=1.16 min (2 min acidic method).

### Synthesis of ethyl 4-(2-aminoethyl)-3,8-dioxo-1-phenyl-2,11,14-trioxa-4,7-diazaheptadecan-17-oate

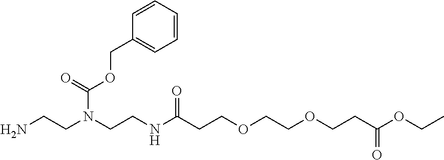 [SMILES: CCOC(=O)CCOCCOCCC(=O)NCCN(CCN)C(=O)OCc1ccccc1]

To a solution of ethyl 7-((benzyloxy)carbonyl)-1-(9H-fluoren-9-yl)-3,11-dioxo-2,14,17-trioxa-4,7,10-triazaicosan-20-oate (395 mg, 0.585 mmol) in EtOH (5 mL) was added dimethylamine in THF (1M, 2.92 mL, 2.92 mmol). The mixture was stirred at RT for 3 h and concentrated. The residue was diluted with DMSO (3 mL) and purified by RP-ISCO chromatography (100 G ISCO Gold C18 Column, Water/MeCN as eluent, 0.1% TFA as modifier). The desired product fractions were combined and lyophilized to afford the title compound (375 mg), which contained impurity. This product was used in the next step without further purification. LC/MS: MH⁺=454.5, Rt=0.78 min (2 min basic method).

### Synthesis of 17-((benzyloxy)carbonyl)-4,13,21-trioxo-3,7,10,24,27,30-hexaoxa-14,17,20-triazatritriacontan-33-oic acid

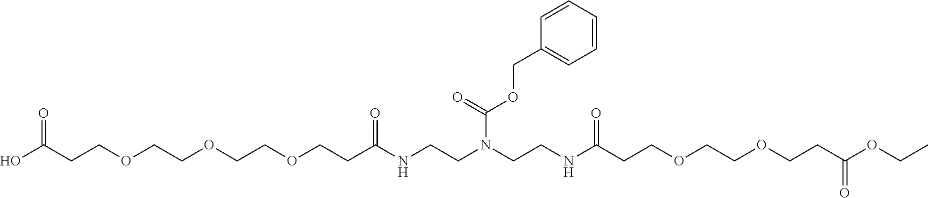 [SMILES: CCOC(=O)CCOCCOCCC(=O)NCCN(CCNC(=O)CCOCCOCCOCCC(=O)O)C(=O)OCc1ccccc1]

A mixture of ethyl 4-(2-aminoethyl)-3,8-dioxo-1-phenyl-2,11,14-trioxa-4,7-diazaheptadecan-17-oate (375 mg from the previous step), 3-(2-(2-(3-((2,5-dioxopyrrolidin-1-yl)oxy)-3-oxopropoxy)ethoxy)ethoxy)propanoic acid (210 mg, 0.605 mmol), and DIPEA (0.840 mL, 4.81 mmol) in DMF (4 mL) was stirred at RT for 5 min. The mixture was diluted with DMSO (5 mL) and loaded on RP-C18 ISCO column (gold, 150g). The column was eluted with MeCN-water (0.1% TFA). Fractions containing the desired product were combined and concentrated to give the title compound as an oil (310 mg), containing a minor impurity. This will be used without further purification. LC/MS: MH⁺=686.6, Rt=0.80 min (2 min acidic method).

### Synthesis of 4,13,21-trioxo-3,7,10,24,27,30-hexaoxa-14,17,20-triazatritriacontan-33-oic acid

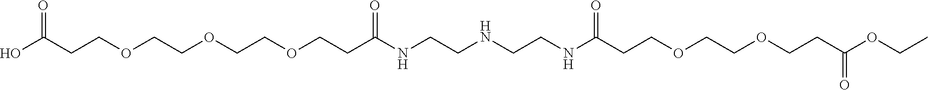 [SMILES: CCOC(=O)CCOCCOCCC(=O)NCCNCCNC(=O)CCOCCOCCOCCC(=O)O]

A mixture of 17-((benzyloxy)carbonyl)-4,13,21-trioxo-3,7,10,24,27,30-hexaoxa-14,17,20-triazatritriacontan-33-oic acid (305 mg, 0.445 mmol) and Pd-C(10% wet) (51 mg, 0.48 mmol) in EtOAc (5 mL) was hydrogenated under balloon pressure at RT for 1 h. The mixture was filtered through Celite with MeCN and water. The filtrate was concentrated to give the crude the title compound as an oil (231 mg), which was directly used in the next step without purification. LC/MS: MH⁺=552.5, Rt=0.50 min (2 min acidic method).

### Synthesis of 17-(3-(2-((tert-butoxycarbonyl)amino)ethoxy)propanoyl)-4,13,21-trioxo-3,7,10,24,27,30-hexaoxa-14,17,20-triazatritriacontan-33-oic acid

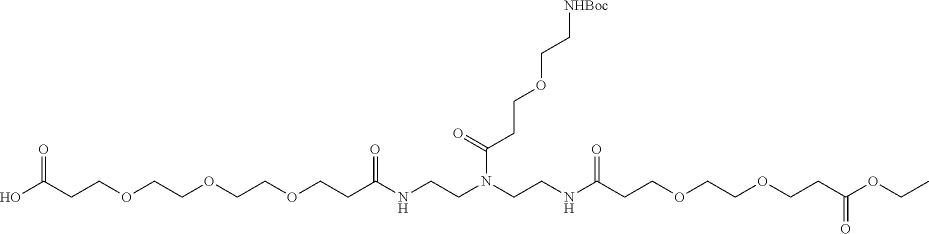 [SMILES: CCOC(=O)CCOCCOCCC(=O)NCCN(CCNC(=O)CCOCCOCCOCCC(=O)O)C(=O)CCOCCNC(=O)OC(C)(C)C]

A mixture of 3-(2-((tert-butoxycarbonyl)amino)ethoxy)propanoic acid (117 mg, 0.503 mmol), HATU (175 mg, 0.461 mmol), and DIPEA (0.30 mL, 1.7 mmol) in DMF (3 mL) was stirred at RT for 10 min. This solution was added to a DMF (1 mL) solution of 4,13,21-trioxo-3,7,10,24,27,30-hexaoxa-14,17,20-triazatritriacontan-33-oic acid (231 mg, 0.419 mmol). DIPEA (0.2 mL) was added. The mixture was stirred at RT for 10 min. The mixture was diluted with DMSO (5 mL) and loaded onto RP-C18 ISCO column (150g, gold). The column was eluted with MeCN-water (0.1% TFA). Fractions containing the desired product were combined and lyophilized to give the product as an oil (163 mg). LC/MS: MH⁺=767.9, Rt=0.77 min (2 min acidic method).

### Synthesis of 2,2,5-trimethyl-4-oxo-3,8,11,14-tetraoxa-5-azaheptadecan-17-oic acid

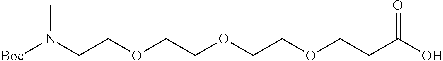 [SMILES: CN(CCOCCOCCOCCC(=O)O)C(=O)OC(C)(C)C]

To a solution of 3-(2-(2-(2-bromoethoxy)ethoxy)ethoxy)propanoic acid (1 g, 3.51 mmol) in THF (10 mL) was added MeNH2 in H₂O, 40% by weight, (26.7 grams, 344 mmol). After stirring overnight, the volatiles were removed in vacuo. The oil was dissolved in MeOH (10 mL) and THF (10 mL), DIEA (0.451 g, 3.51 mmol) was added followed by Boc20 (2.296 g, 10.5 mmol). After 2.5 hours, the volatiles were removed in vacuo, the residue was dissolved in DMSO and purified by ISCO RP-HPLC. Upon lyophilization, 2,2,5-trimethyl-4-oxo-3,8,11,14-tetraoxa-5-azaheptadecan-17-oic acid (1.10 g, 94% yield) was obtained. LC/MS: MH⁺=336.4, Rt=1.57 min (5 min acidic run).

### Synthesis of 2-cyanoethyl 5,8,11-trioxa-2-azatetradecan-14-oate

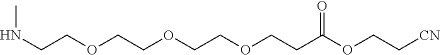 [SMILES: CNCCOCCOCCOCCC(=O)OCCC#N]

To a solution of 2,2,5-trimethyl-4-oxo-3,8,11,14-tetraoxa-5-azaheptadecan-17-oic acid (4.56 grams, 13.6 mmol), DMAP (332 mg, 2.72 mmol) and 3-hydroxypropanenitrile (0.996 mL, 14.28 mmol) in CH₂Cl₂ (100 mL) was added EDC (3.128 grams, 16.31 mmol). After stirring for 16 hours, the reactions were partitioned between EtOAc and H₂O, mixed, separated, washed with 1M HCl, NaHCO₃(sat), NaCl(sat.), dried over MgSO₄, filtered and concentrated. The residue was treated with 25% TFA/CH₂Cl₂ (24 mL) for 1.5 hours at which time the volatiles were removed in vacuo and pumped on to yield 8.09 grams of 2-cyanoethyl 5,8,11-trioxa-2-azatetradecan-14-oate as trifluoroacetate salt. Based on weight, assumed 2.65 equivalents of TFA and used as such. LC/MS: MH⁺=289.2, Rt=0.48 min (5 min acidic method).

### Synthesis of allyl 5,8,11-trioxa-2-azatetradecan-14-oate

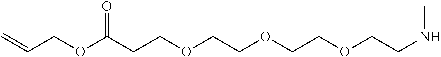 [SMILES: C=CCOC(=O)CCOCCOCCOCCNC]

To a solution of 2,2,5-trimethyl-4-oxo-3,8,11,14-tetraoxa-5-azaheptadecan-17-oic acid (3.026 grams, 9.022 mmol), DMAP (331 mg, 2.71 mmol) and allyl alcohol (0.689 mL, 9.92 mmol) in CH₂C12 (50 mL) was added EDC (2.08 grams, 10.83 mmol). After stirring for 2 hours, the reactions were partitioned between EtOAc and H₂O, mixed, separated, washed with 1M HCl, NaHCO₃(sat), NaCl(sat.), dried over MgSO₄, filtered and concentrated. The residue was treated with 4M HCl in dioxane (24 mL, 96 mmol). After standing at rt for 14 hours, the volatiles were removed in vacuo and dissolved in MeCN/H₂O. Upon lyophilization, allyl 5,8,11-trioxa-2-azatetradecan-14-oate (2.55 grams, 82% yield) was obtained as HCl salt. LC/MS: MH⁺=276.3, Rt=0.72 min (5 min acidic method).

### Synthesis of 3-(tert-butoxycarbonyl)-6-methyl-5,18-dioxo-9,12,15,19-tetraoxa-3,6-diazadocos-21-enoic acid

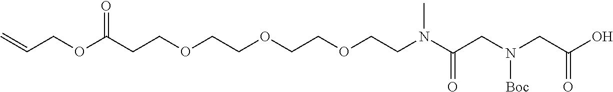 [SMILES: C=CCOC(=O)CCOCCOCCOCCN(C)C(=O)CN(CC(=O)O)C(=O)OC(C)(C)C]

To a solution of allyl 5,8,11-trioxa-2-azatetradecan-14-oate (1.944 grams, 9.03 mmol) and tert-butyl 3,5-dioxopiperidine-1-carboxylate HCl salt (2.817 grams, 9.03 mmol) in DMF (5 mL) was added DIEA (3.15 mL, 18.07 mmol). After stirring at rt for 2 hours, the solution was diluted with DMSO and purified by RP-ISCO. Upon lyophilization, 3-(tert-butoxycarbonyl)-6-methyl-5,18-dioxo-9,12,15,19-tetraoxa-3,6-diazadocos-21-enoic acid (2.62 grams, 59% yield) was obtained. LC/MS: MH⁺=491.5, Rt=1.85 min (5 min acidic method).

### Synthesis of 1-allyl 31-(2-cyanoethyl) 16-(tert-butoxycarbonyl)-13,19-dimethyl-14,18-dioxo-4,7,10,22,25,28-hexaoxa-13,16,19-triazahentriacontanedioate

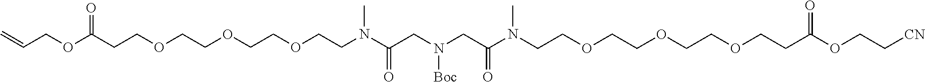 [SMILES: C=CCOC(=O)CCOCCOCCOCCN(C)C(=O)CN(CC(=O)N(C)CCOCCOCCOCCC(=O)OCCC#N)C(=O)OC(C)(C)C]

To a solution of 2-cyanoethyl 5,8,11-trioxa-2-azatetradecan-14-oate trifluoroacetate(2.65 equiv) (3.406 grams, 5.768 mmol) in CH₂Cl₂ (10 mL) was added DIEA (4.65 mL, 26.7 mmol). The solution was added to 3-(tert-butoxycarbonyl)-6-methyl-5,18-dioxo-9,12,15,19-tetraoxa-3,6-diazadocos-21-enoic acid (2.620 grams, 5.34 mmol) and HOAT (873 mg, 6.41 mmol) and EDC (1.229 grams, 6.41 mmol) was then added last. After stirring at rt for 5 hours, the volatiles were removed in vacuo, the residue was dissolved in DMSO and purified by RP-ISCO. After lyophilization, 1-allyl 31-(2-cyanoethyl) 16-(tert-butoxycarbonyl)-13,19-dimethyl-14,18-dioxo-4,7,10,22,25,28-hexaoxa-13,16,19-triazahentriacontanedioate (3.57 grams, 87% yield) was obtained. LC/MS: MH⁺=761.7, Rt=1.95 min (5 min acidic method).

### Synthesis of 1-allyl 31-(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate

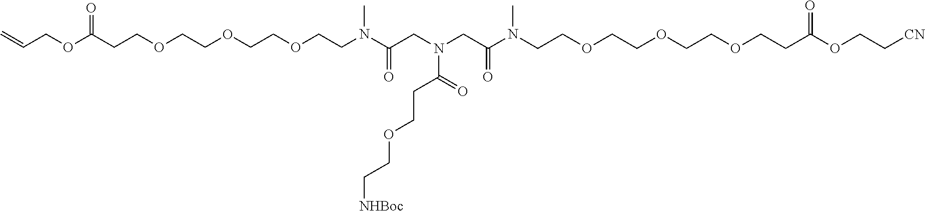 [SMILES: C=CCOC(=O)CCOCCOCCOCCN(C)C(=O)CN(CC(=O)N(C)CCOCCOCCOCCC(=O)OCCC#N)C(=O)CCOCCNC(=O)OC(C)(C)C]

1-allyl 31-(2-cyanoethyl) 16-(tert-butoxycarbonyl)-13,19-dimethyl-14,18-dioxo-4,7,10,22,25,28-hexaoxa-13,16,19-triazahentriacontanedioate (803 mg, 1.06 mmol) was treated with 4M HCl in dioxane (5 mL, 40 μmol) for 12 hours at which time the volatiles were removed in vacuo. The residue was dissolved in NMP (3 mL) and was neutralized by adding DIEA (0.5 mL, 2.87 mmol). 3-(2-((tert-butoxycarbonyl)amino)ethoxy)propanoic acid (320 mg, 1.37 mmol), HOAT (187 mg, 1.37 mmol) and EDC (263 mg, 1.37 mmol) were added and after stirring at rt for 2 hours the solution was diluted with DMSO and purified by RP-ISCO. After lyophilization, 1-allyl 31-(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (657 mg, 71%) was obtained. LC/MS: MH⁺=876.7, Rt=2.07 min (5 min acidic method).

### Synthesis of 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid

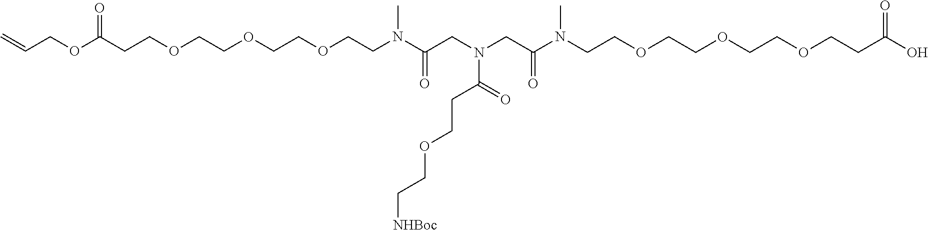 [SMILES: C=CCOC(=O)CCOCCOCCOCCN(C)C(=O)CN(CC(=O)N(C)CCOCCOCCOCCC(=O)O)C(=O)CCOCCNC(=O)OC(C)(C)C]

To a solution of 1-allyl 31-(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (375 mg, 428 μmol) in DMSO (1 mL) was added DBU (129 μL, 856 μmol). After standing for two hours, the solution was purified by ISCO C18 RP-HPLC with 0.05% formic acid modifier. After lyophilization, 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (232 mg, 66% yield) was obtained. LC/MS: MH⁺=823.7, Rt=1.86 min (5 min acidic method).

### Synthesis of tert-butyl 11-(2-(tert-butoxy)-2-oxoethyl)-1-(9H-fluoren-9-yl)-3,10-dioxo-2,7-dioxa-4,11-diazatridecan-13-oate

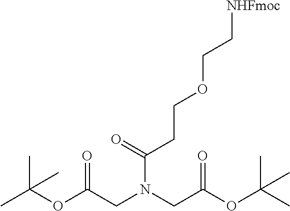

To a solution of di-tert-butyl 2,2′-azanediyldiacetate (250 mg, 1019 μmol) and HATU (407 mg, 1070 μmol) in DMF (2 mL) was added DIEA (356 μL, 2038 μmol). After stirring for 5 minutes, 3-(2-((((9H-fluoren-9-yl)methoxy)carbonyl)amino)ethoxy)propanoic acid (398 mg, 1121 μmol) was added. After stirring for an additional 30 minutes, the solution was diluted with DMSO and purified by ISCO RP-HPLC. Upon lyophilization, tert-butyl 11-(2-(tert-butoxy)-2-oxoethyl)-1-(9H-fluoren-9-yl)-3,10-dioxo-2,7-dioxa-4,11-diazatridecan-13-oate (509 mg, 86% yield) was obtained. LC/MS: MH⁺=583.4, Rt=3.06 min (5 min acidic method).

### Synthesis of 11-(carboxymethyl)-1-(9H-fluoren-9-yl)-3,10-dioxo-2,7-dioxa-4,11-diazatridecan-13-oic acid

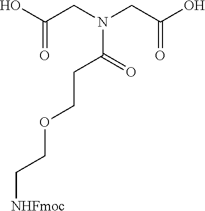

Tert-butyl 11-(2-(tert-butoxy)-2-oxoethyl)-1-(9H-fluoren-9-yl)-3,10-dioxo-2,7-dioxa-4,11-diazatridecan-13-oate (509 mg, 874 μmol) was treated with 33% TFA/CH₂Cl₂ (3 mL) for 4 hours at which time the volatiles were removed in vacuo. The residue was dissolved in MeCN/H₂O and upon lyophilization 11-(carboxymethyl)-1-(9H-fluoren-9-yl)-3,10-dioxo-2,7-dioxa-4,11-diazatridecan-13-oic acid (400 mg, 97% yield) was obtained. LC/MS: MH⁺=471.0, Rt=1.87 min (5 min acidic method).

### Synthesis of di-tert-butyl 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate

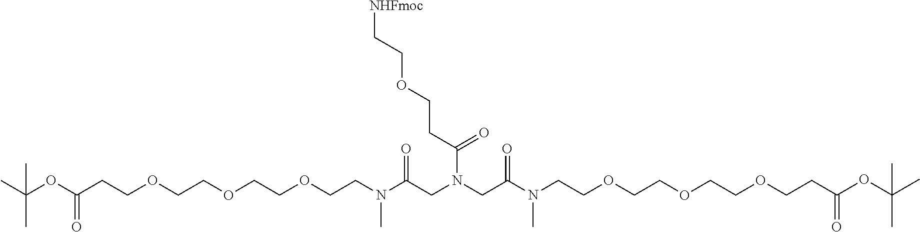

To a solution of 11-(carboxymethyl)-1-(9H-fluoren-9-yl)-3,10-dioxo-2,7-dioxa-4,11-diazatridecan-13-oic acid (40 mg, 85 μmol) and HATU (71 mg, 187 μmol) in DMF (2 mL) was added DIEA (74 μL, 74 μmol). After stirring for 5 minutes, tert-butyl 5,8,11-trioxa-2-azatetradecan-14-oate (74 mg, 255 μmol) was added. After stirring for an additional 30 minutes, the solution was diluted with DMSO and purified by ISCO RP-HPLC. Upon lyophilization, di-tert-butyl 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate (54 mg, 62% yield) was obtained. LC/MS: MH⁺=1017.8, Rt=3.01 min (5 min acidic method).

### Synthesis of 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioic acid

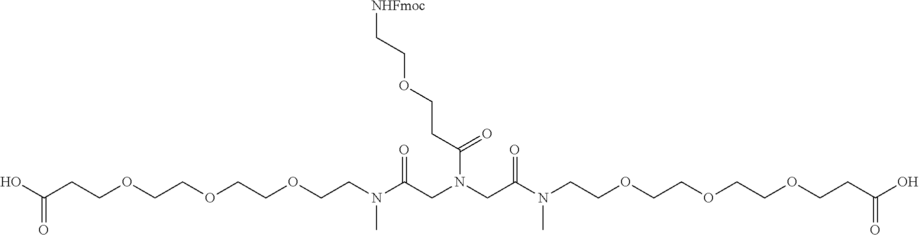

Di-tert-butyl 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate (54 mg, 53 μmol) was treated with 33% TFA/CH₂Cl₂ (3 mL) for 4 hours at which time the volatiles were removed in vacuo. The residue was dissolved in MeCN/H₂O and upon lyophilization 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioic acid (47.4 mg, 99% yield) was obtained. HRMS: MH⁺=905.4400, Rt=2.04 min (5 min acidic method).

### Synthesis of di-tert-butyl 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioate

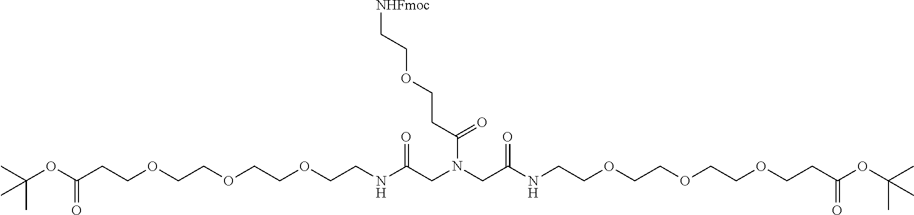

To a solution of 11-(carboxymethyl)-1-(9H-fluoren-9-yl)-3,10-dioxo-2,7-dioxa-4,11-diazatridecan-13-oic acid (100 mg, 213 μmol) and HATU (162 mg, 425 μmol) in DMF (2 mL) was added DIEA (111 μL, 638 μmol). After stirring for 5 minutes, tert-butyl 3-(2-(2-(2-aminoethoxy)ethoxy)ethoxy)propanoate (177 mg, 638 μmol) was added. After stirring for an additional 30 minutes, the solution was diluted with DMSO and purified by ISCO RP-HPLC. Upon lyophilization, di-tert-butyl 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioate (185 mg, 88% yield) was obtained. LC/MS: MH⁺=989.5300, Rt=2.77 min (5 min acidic method).

### Synthesis of 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioic acid

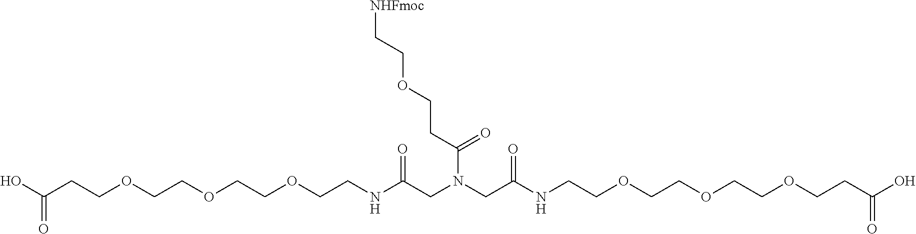

Di-tert-butyl 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioate (185 mg, 187 μmol) was treated with 50% TFA/CH₂Cl₂ (44 mL) for 2 hours at which time the volatiles were removed in vacuo. The residue was dissolved in MeCN/H₂O and upon lyophilization 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioic acid (146 mg, 89% yield) was obtained. LC/MS: MH⁺=877.6, Rt=1.91 min (5 min acidic method).

### Synthesis of tert-butyl (11-(2-((tert-butoxycarbonyl)amino)ethyl)-1-(9H-fluoren-9-yl)-3,10-dioxo-2,7-dioxa-4,11-diazatridecan-13-yl)carbamate

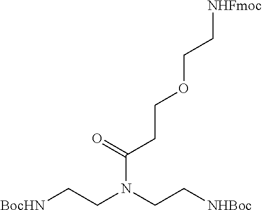

To a solution of 3-(2-((((9H-fluoren-9-yl)methoxy)carbonyl)amino)ethoxy)propanoic acid (322 mg, 906 μmol) and HATU (329 mg, 865 μmol) in DMF (2 mL) was added DIEA (288 p L, 1648 μmol). After stirring for 5 minutes, di-tert-butyl (azanediylbis(ethane-2,1-diyl))dicarbamate (250 mg, 824 μmol) was added. After stirring for an additional 30 minutes, the solution was diluted with DMSO and purified by ISCO RP-HPLC. Upon lyophilization, tert-butyl (11-(2-((tert-butoxycarbonyl)amino)ethyl)-1-(9H-fluoren-9-yl)-3,10-dioxo-2,7-dioxa-4,11-diazatridecan-13-yl)carbamate (528 mg, 85% yield) was obtained. LC/MS: MH⁺=641.4, Rt=2.82 min (5 min acidic method).

### Synthesis of (9H-fluoren-9-yl)methyl (2-(3-(bis(2-aminoethyl)amino)-3-oxopropoxy)ethyl)carbamate

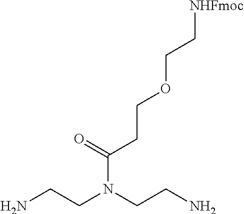

tert-butyl (11-(2-((tert-butoxycarbonyl)amino)ethyl)-1-(9H-fluoren-9-yl)-3,10-dioxo-2,7-dioxa-4,11-diazatridecan-13-yl)carbamate (450 mg, 702 μmol) was treated with 25% TFA/CH₂Cl₂ (3 mL) for 1 hour at which time the volatiles were removed in vacuo. The residue was dissolved in MeCN/H₂O and upon lyophilization (9H-fluoren-9-yl)methyl (2-(3-(bis(2-aminoethyl)amino)-3-oxopropoxy)ethyl)carbamate (480 mg) was obtained. LC/MS: MH⁺=441.1, Rt=0.94 min (5 min acidic method).

### Synthesis of tert-buty 111-(2-aminoethyl)-1-(9H-fluoren-9-yl)-3,10,15-trioxo-2,7,18,21-tetraoxa-4,11,14-triazatetracosan-24-oate

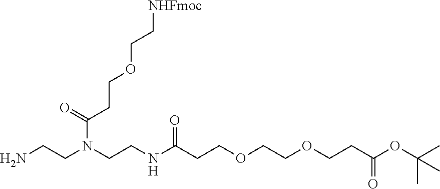

To a solution of 3-(2-(3-(tert-butoxy)-3-oxopropoxy)ethoxy)propanoic acid (30 mg, 114 μmol) and HATU (43.5 mg, 114 μmol) in DMF (2 mL) was added DIEA (120 μL, 686 μmol). After stirring for 5 minutes, (9H-fluoren-9-yl)methyl (2-(3-(bis(2-aminoethyl)amino)-3-oxopropoxy)ethyl)carbamate TFA salt (308 mg, 343 μmol) was added. After stirring for an additional 30 minutes, the solution was diluted with DMSO and purified by ISCO RP-HPLC. Upon lyophilization, tert-butyl 11-(2-aminoethyl)-1-(9H-fluoren-9-yl)-3,10,15-trioxo-2,7,18,21-tetraoxa-4,11,14-triazatetracosan-24-oate (65 mg, 62% yield) was obtained. LC/MS: MH⁺=685.5, Rt=2.05 min (5 min acidic method).

Synthesis of di-tert-butyl 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioate

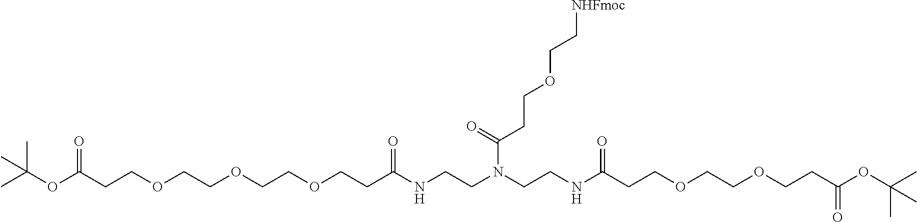

To a solution of 2,2-dimethyl-4-oxo-3,7,10,13-tetraoxahexadecan-16-oic acid (26 mg, 85 μmol) and HATU (32.2 mg, 85 μmol) in DMF (1 mL) was added DIEA (62 μL, 353 μmol). After stirring for 5 minutes, tert-butyl 11-(2-aminoethyl)-1-(9H-fluoren-9-yl)-3,10,15-trioxo-2,7,18,21-tetraoxa-4,11,14-triazatetracosan-24-oate TFA salt (65 mg, 71 μmol) was added. After stirring for an additional 30 minutes, the solution was diluted with DMSO and purified by ISCO RP-HPLC. Upon lyophilization, di-tert-butyl 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioate (46 mg, 67% yield) was obtained. LC/MS: MH⁺=973.8, Rt=2.84 min (5 min acidic method).

### Synthesis of 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioic acid

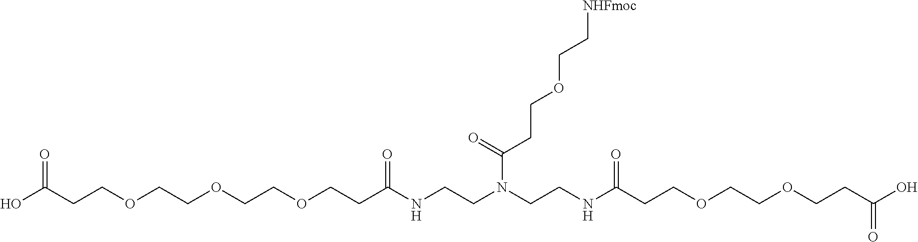

Di-tert-butyl 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioate (46.4 mg, 48 μmol) was treated with 50% TFA/CH₂Cl₂ (2 mL) for 2 hours at which time the volatiles were removed in vacuo. The residue was dissolved in MeCN/H₂O and upon lyophilization 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioic acid (46 mg, 99% yield) was obtained. LC/MS: MH⁺=861.4, Rt=1.87 min (5 min acidic method).

### Synthesis of di-tert-butyl 10-(3-(2-((((9H-fluoren-9-yl)methoxy)carbonyl)amino)ethoxy)propanoyl)-8,12-dioxo-4,16-dioxa-7,10,13-triazanonadecanedioate

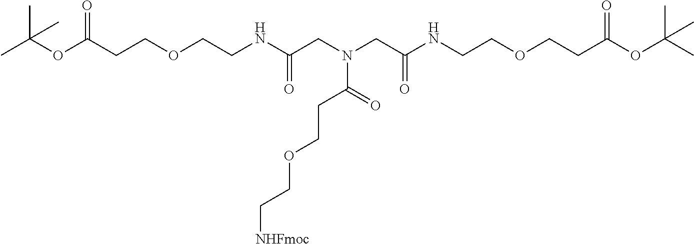

To a solution of 11-(carboxymethyl)-1-(9H-fluoren-9-yl)-3,10-dioxo-2,7-dioxa-4,11-diazatridecan-13-oic acid (50 mg, 106 μmol) and HATU (81 mg, 213 μmol) in DMF (2 mL) was added DIEA (93 μL, 531 μmol). After stirring for 45 minutes, tert-butyl 3-(2-aminoethoxy)propanoate (60.3 mg, 319 μmol) was added. After stirring for an additional 60 minutes, the solution was diluted with DMSO and purified by ISCO RP-HPLC. Upon lyophilization, di-tert-butyl 10-(3-(2-((((9H-fluoren-9-yl)methoxy)carbonyl)amino)ethoxy)propanoyl)-8,12-dioxo-4,16-dioxa-7,10,13-triazanonadecanedioate (44 mg, 51% yield) was obtained. LC/MS: MH⁺=813.7, Rt=2.95 min (5 min acidic method).

### Synthesis of 10-(3-(2-((((9H-fluoren-9-yl)methoxy)carbonyl)amino)ethoxy)propanoyl)-8,12-dioxo-4,16-dioxa-7,10,13-triazanonadecanedioic acid

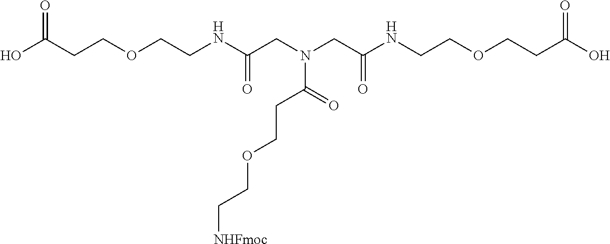

Di-tert-butyl 10-(3-(2-((((9H-fluoren-9-yl)methoxy)carbonyl)amino)ethoxy)propanoyl)-8,12-dioxo-4,16-dioxa-7,10,13-triazanonadecanedioate (44 mg, 54 μmol) was treated with 33% TFA/CH₂Cl₂ (3 mL) for 1 hour at which time the volatiles were removed in vacuo. The residue was dissolved in MeCN/H₂O and upon lyophilization 10-(3-(2-((((9H-fluoren-9-yl)methoxy)carbonyl)amino)ethoxy)propanoyl)-8,12-dioxo-4,16-dioxa-7,10,13-triazanonadecanedioic acid (47 mg) was obtained. LC/MS: MH⁺=701.4, Rt=1.76 min (5 min acidic method).

### Synthesis of 1-(tert-butyl) 18-methyl 3-(((9H-fluoren-9-yl)methoxy)carbonyl)-5-oxo-9,12,15-trioxa-3,6-diazaoctadecanedioate

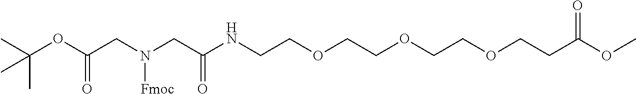

To a solution of N-(((9H-fluoren-9-yl)methoxy)carbonyl)-N-(2-(tert-butoxy)-2-oxoethyl)glycine (1 g, 2.43 mmol) and HATU (970 mg, 2.55 mmol) in DMF (12 mL) was added DIEA (1.7 mL, 9.7 mmol). After stirring for 5 minutes, methyl 3-(2-(2-(2-aminoethoxy)ethoxy)ethoxy)propanoate (899 mg, 2.67 mmol) was added. After stirring for an additional 30 minutes, the solution was poured into NH4Cl(sat.), was extracted with EtOAc and concentrated. Upon purification by SiO₂ chromatography and concentration, 1-(tert-butyl) 18-methyl 3-(((9H-fluoren-9-yl)methoxy)carbonyl)-5-oxo-9,12,15-trioxa-3,6-diazaoctadecanedioate (1.11 g, 73% yield) was obtained. LC/MS: MH⁺=629.5 and (M-tBu)+=573.4, Rt=2.70 min (5 min acidic method).

### Synthesis of 18-(((9H-fluoren-9-yl)methoxy)carbonyl)-3,16-dioxo-2,6,9,12-tetraoxa-15,18-diazaicosan-20-oic acid

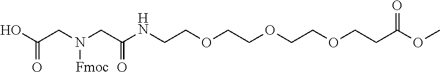

1-(tert-butyl) 18-methyl 3-(((9H-fluoren-9-yl)methoxy)carbonyl)-5-oxo-9,12,15-trioxa-3,6-diazaoctadecanedioate (1.11 g, 1.77 mol) was treated with 33% TFA/CH₂Cl₂ (6 mL) for 1 hour at which time dichloroethane (50 mL) was added and the volatiles were removed in vacuo. The material was used as is in the next step. LC/MS: MH⁺=573.4, Rt=0.96 min (2 min acidic method). Synthesis of 1-(tert-butyl) 31-methyl 16-(((9H-fluoren-9-yl)methoxy)carbonyl)-14,18-dioxo-4,7,10,22,25,28-hexaoxa-13,16,19-triazahentriacontanedioate

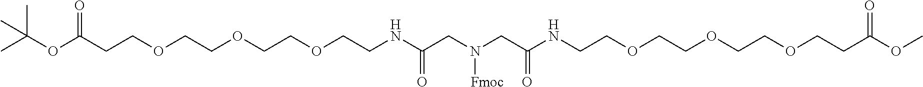

To a solution of 18-(((9H-fluoren-9-yl)methoxy)carbonyl)-3,16-dioxo-2,6,9,12-tetraoxa-15,18-diazaicosan-20-oic acid (1.33 g, 1.66 mmol) and HATU (695 mg, 1.83 mmol) in DMF (10 mL) was added DIEA (1.16 mL, 6.64 mmol). After stirring for 5 minutes, tert-butyl 3-(2-(2-(2-aminoethoxy)ethoxy)ethoxy)propanoate (599 mg, 2.16 mmol) was added. After stirring for an additional 30 minutes, the solution was poured into NH4Cl(sat.), was extracted with EtOAc and concentrated. Upon purification by SiO₂ chromatography and concentration, 1-(tert-butyl) 31-methyl 16-(((9H-fluoren-9-yl)methoxy)carbonyl)-14,18-dioxo-4,7,10,22,25,28-hexaoxa-13,16,19-triazahentriacontanedioate (1.19 g, 86% yield) was obtained. LC/MS: MH⁺=832.6, Rt=1.14 min (2 min acidic method).

### Synthesis of 18-(((9H-fluoren-9-yl)methoxy)carbonyl)-3,16,20-trioxo-2,6,9,12,24,27,30-heptaoxa-15,18,21-triazatritriacontan-33-oic acid

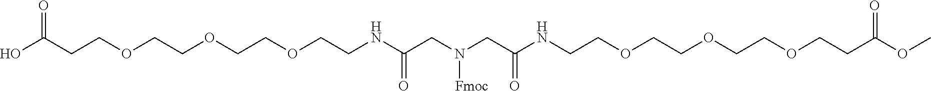

1-(tert-butyl) 31-methyl 16-(((9H-fluoren-9-yl)methoxy)carbonyl)-14,18-dioxo-4,7,10,22,25,28-hexaoxa-13,16,19-triazahentriacontanedioate (1.19 g, 1.46 mmol) was treated with 33% TFA/CH₂Cl₂ (4.5 mL) for one hour at which time the volatiles were removed in vacuo. The residue was dissolved in MeCN/H₂O and upon lyophilization 18-(((9H-fluoren-9-yl)methoxy)carbonyl)-3,16,20-trioxo-2,6,9,12,24,27,30-heptaoxa-15,18,21-triazatritriacontan-33-oic acid was obtained. LC/MS: MH⁺=776.6, Rt=0.94 min (2 min acidic method Synthesis of 3,16,20-trioxo-2,6,9,12,24,2,30-heptaxa-15,18,21-triazatritriacontan-33-oic acid

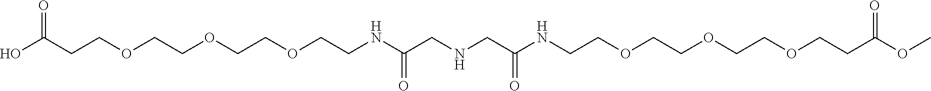 [SMILES: COC(=O)CCOCCOCCOCCNC(=O)CNCC(=O)NCCOCCOCCOCCC(=O)O]

A solution of 18-(((9H-fluoren-9-yl)methoxy)carbonyl)-3,16,20-trioxo-2,6,9,12,24,27,30-heptaoxa-15,18,21-triazatritriacontan-33-oic acid (1.47 g, 1.9 mmol) and 2.0 M dimethyl amine in THF (7 mL) was stirred at rt for one hour at which time the volatiles were removed in vacuo. The residue was dissolved in MeCN and neutralized with TFA (150 μL). The volatiles were removed in vacuo, the residue was dissolved in DMSO and purified by ISCO RP-HPLC. Upon lyophilization, 3,16,20-trioxo-2,6,9,12,24,27,30-heptaoxa-15,18,21-triazatritriacontan-33-oic acid (744 mg, 59% yield) was obtained. LC/MS: MH⁺=554.5, Rt=0.72 min (5 min acidic method).

### Synthesis of 18-(3-(2-((tert-butoxycarbonyl)amino)ethoxy)propanoyl)-3,16,20-trioxo-2,6,9,12,24,27,30-heptaoxa-15,18,21-triazatritriacontan-33-oic acid

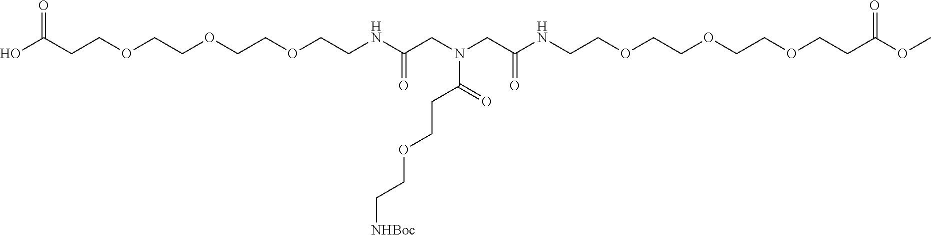 [SMILES: COC(=O)CCOCCOCCOCCNC(=O)CN(CC(=O)NCCOCCOCCOCCC(=O)O)C(=O)CCOCCNC(=O)OC(C)(C)C]

To a solution of 3-(2-((tert-butoxycarbonyl)amino)ethoxy)propanoic acid (470 mg, 2.02 mmol) and HATU (613 mg, 1.61 mmol) in DMF (6 mL) was added DIEA (704 μL, 4.03 mmol). After stirring for 5 minutes, 3,16,20-trioxo-2,6,9,12,24,27,30-heptaoxa-15,18,21-triazatritriacontan-33-oic acid (744 mg, 1.34 mmol) was added. After stirring for an additional 30 minutes, the solution was diluted with DMSO and purified by ISCO RP-HPLC. Upon lyophilization, 18-(3-(2-((tert-butoxycarbonyl)amino)ethoxy)propanoyl)-3,16,20-trioxo-2,6,9,12,24,27,30-heptaoxa-15,18,21-triazatritriacontan-33-oic acid (790 mg, 76% yield) was obtained. LC/MS: MH⁺=769.8, Rt=1.54 min (5 min acidic method).

### Synthesis of (9H-fluoren-9-yl)methyl (3-(bis(2-hydroxyethyl)amino)-3-oxopropyl)carbamate

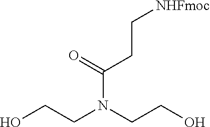

To a stirred solution of 3-((((9H-fluoren-9-yl)methoxy)carbonyl)amino)propanoic acid (2.96 g, 9.51 mmol, 1.00 equiv.) in dichloromethane (50 mL) was added 3-(((ethylimino)methylene)amino)-N,N-dimethylpropan-1-amine hydrochloride (2.006 g, 10.46 mmol, 1.10 equiv.). After stirring for 10 minutes at ambient temperature, diethanolamine (1.00 g, 9.51 mmol, 1.00 equiv.) was added and the resulting mixture was stirred at ambient temperature for 2 hours. The reaction mixture was concentrated under reduced pressure and the resulting residue was purified by chromatography on silica gel (0-20% methanol / dichloromethane) to afford (9H-fluoren-9-yl)methyl (3-(bis(2-hydroxyethyl)amino)-3-oxopropyl)carbamate (2.25 g, 5.65 mmol, 59% yield). LC/MS: MH⁺=399.3, Rt=0.82 min (2 min acidic run).

### Synthesis of (9H-fluoren-9-yl)methyl (3-(bis(2-(((4-nitrophenoxy)carbonyl)oxy)ethyl)amino)-3-oxopropyl)carbamate

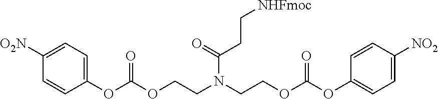

To a stirred solution of (9H-fluoren-9-yl)methyl (3-(bis(2-hydroxyethyl)amino)-3-oxopropyl)carbamate (2.24 g, 5.62 mmol, 1.00 equiv.) and bis(4-nitrophenyl) carbonate (3.76 g, 12.37 mmol, 2.20 equiv.) in dichloromethane (50 mL) was added N-ethyldiisopropylamine (1.60 g, 12.37 mmol, 2.20 equiv.). The resulting mixture was stirred at ambient temperature for 2 hours. The reaction mixture was concentrated under reduced pressure at or below ambient temperature, and the resulting residue was purified by chromatography on silica gel (0-100% ethyl acetate/heptanes) to afford (9H-fluoren-9-yl)methyl (3-(bis(2-(((4-nitrophenoxy)carbonyl)oxy)ethyl)amino)-3-oxopropyl)carbamate (2.00 g, 2.74 mmol, 48% yield). LC/MS: M⁺Na*=751.3, Rt=1.20 min (2 min acidic run). ¹H NMR (400 MHz, Chloroform-d) δ 8.27-8.22 (m, 2H), 8.22-8.18 (m, 2H), 7.74 (d, J=7.4 Hz, 2H), 7.53 (d, J=7.4 Hz, 2H), 7.42-7.33 (m, 4H), 7.32-7.27 (m, 4H), 5.51 (t, J=6.3 Hz, 1H), 4.46 (dt, J=18.3, 5.3 Hz, 4H), 4.30 (d, J=7.2 Hz, 2H), 3.80 (dt, J=10.2, 5.4 Hz, 4H), 3.56 (d, J=5.8 Hz, 2H), 2.71 (t, J=5.5 Hz, 2H).

### Synthesis of tert-butyl (1-hydroxy-9-(2-(2-(2-hydroxyethoxy)ethoxy)ethyl)-10-oxo-3,6,13-trioxa-9-azapentadecan-15-yl)carbamate

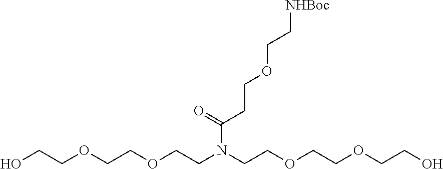 [SMILES: CC(C)(C)OC(=O)NCCOCCC(=O)N(CCOCCOCCO)CCOCCOCCO]

To a stirred solution of 3,6,12,15-tetraoxa-9-azaheptadecane-1,17-diol (116 mg, 0.412 mmol, 1.00 equiv.), 3-(2-((tert-butoxycarbonyl)amino)ethoxy)propanoic acid (101 mg, 0.433 mmol, 1.05 equiv.), and HATU (165 mg, 0.433 mmol, 1.05 equiv.) in DMF (1 mL) was added N-ethyldiisopropylamine (160 mg, 1.23 mmol, 3.00 equiv.). The resulting mixture was stirred at ambient temperature for 1 hour, then diluted with DMSO (2 mL) and purified by RP-ISCO. Upon lyophilization, tert-butyl (1-hydroxy-9-(2-(2-(2-hydroxyethoxy)ethoxy)ethyl)-10-oxo-3,6,13-trioxa-9-azapentadecan-15-yl)carbamate (118 mg, 0.238 mmol, 57% yield) was obtained. LC/MS: MH⁺=497.4, Rt=0.68 min (2 min acidic run).

### Synthesis of tert-butyl (6-oxo-7-(2-(2-(2-(prop-2-yn-1-yloxy)ethoxy)ethoxy)ethyl)-3,10,13,16-tetraoxa-7-azanonadec-18-yn-1-yl)carbamate

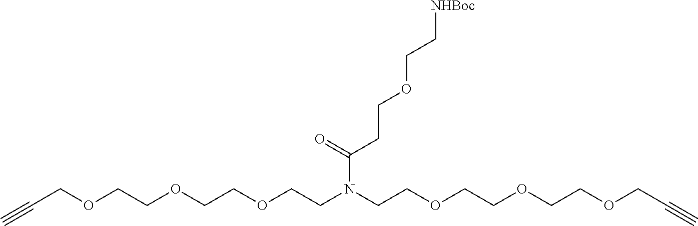 [SMILES: C#CCOCCOCCOCCN(CCOCCOCCOCC#C)C(=O)CCOCCNC(=O)OC(C)(C)C]

To a stirred solution of tert-butyl (1-hydroxy-9-(2-(2-(2-hydroxyethoxy)ethoxy)ethyl)-10-oxo-3,6,13-trioxa-9-azapentadecan-15-yl)carbamate (118 mg, 0.238 mmol, 1.00 equiv.) in dry THF (2 mL) at 0° C. was added sodium hydride (60% dispersion in oil, 20.0 mg, 0.499 mmol, 2.1 equiv.). After stirring at 0° C. for 30 minutes, propargyl bromide (62.2 mg, 0.523 mmol, 2.2 equiv.) was added and the resulting mixture was allowed to warm to ambient temperature with stirring over 20 hours. After concentration under reduced pressure, the resulting residue was purified by chromatography on silica gel (0-20% isopropanol/dichloromethane) to afford tert-butyl (6-oxo-7-(2-(2-(2-(prop-2-yn-1-yloxy)ethoxy)ethoxy)ethyl)-3,10,13,16-tetraoxa-7-azanonadec-18-yn-1-yl)carbamate (12.6 mg, 0.022 mmol, 9.3% yield). LC/MS: MH⁺=573.4, Rt=0.95 min (2 min acidic run). 1H NMR (400 MHz, Chloroform-d) δ 4.19 (d, J=2.3 Hz, 4H), 3.74 (t, J=6.4 Hz, 2H), 3.71-3.54 (m, 24H), 3.50 (t, J=5.1 Hz, 2H), 3.28 (d, J=5.7 Hz, 2H), 2.67 (t, J=6.4 Hz, 2H), 2.44 (t, J=2.4 Hz, 2H), 1.44 (d, J=2.9 Hz, 9H).

### Synthesis of Tert-butyl (6-oxo-7-(2-(2-(prop-2-yn-1-yloxy)ethoxy)ethyl)-3,10,13-trioxa-7-azahexadec-15-yn-1-yl)carbamate

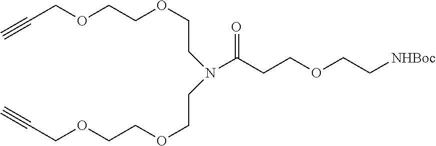 [SMILES: C#CCOCCOCCN(CCOCCOCC#C)C(=O)CCOCCNC(=O)OC(C)(C)C]

To the solution of NH-bis(PEG2-propargyl) (53 mg, 0.197 mmol), t-Boc-N-amido-PEG1-acid (48.2 mg, 0.207 mmol) in DMF (dry, 1 ml) was added HATU (79 mg, 0.207 mmol) and then DIPEA (103 μl, 0.590 mmol). The reaction mixture was stirred at RT for 1 h. The crude was purified by C-18 Column (50 g cartridge, eluted with MeCN/Water with 0.1% Formic Acid, 0-100% over 16 CV) to obtain Tert-butyl (6-oxo-7-(2-(2-(prop-2-yn-1-yloxy)ethoxy)ethyl)-3,10,13-trioxa-7-azahexadec-15-yn-1-yl)carbamate (81 mg, 85% yield). LC/MS: MH⁺=485.4, Rt=0.94 min (3 min acidic run).

### Synthesis of 3-(2-aminoethoxy)-N,N-bis(2-(2-(prop-2-yn-1-yloxy)ethoxy)ethyl)propanamide

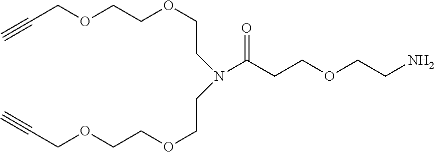 [SMILES: C#CCOCCOCCN(CCOCCOCC#C)C(=O)CCOCCN]

At 0° C. in an ice-water bath, to tert-butyl (6-oxo-7-(2-(2-(prop-2-yn-1-yloxy)ethoxy)ethyl)-3,10,13-trioxa-7-azahexadec-15-yn-1-yl)carbamate (81 mg, 0.167 mmol) was added TFA (25% in DCM) (2576 μl, 8.36 mmol). Then the reaction mixture was raised to RT and stirred for 90 min. The mixture was concentrated under high vacuum at RT bath. Then the crude was separated with C-18 column (50 gram cartridge, MeCN/Water with 0.1% Formic acid 0-100% over 16CV) to obtain 3-(2-aminoethoxy)-N,N-bis(2-(2-(prop-2-yn-1-yloxy)ethoxy)ethyl)propanamide (60 mg, 83% yield). LC/MS: MH⁺=385.8, Rt=0.56 min (3 min acidic run).

### Synthesis of Tert-butyl (S)-(17-oxo-4,7,10,13-tetraoxa-16-azahenicosa-1,20-diyn-18-yl)carbamate

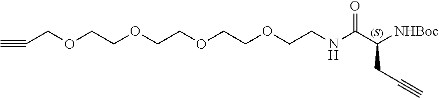

To a mixture of N-Boc-2-Propargyl-L-Glycine (100 mg, 0.469 mmol), Propargyl-PEG4-Amine (108 mg, 0.469 mmol), and TSTU (141 mg, 0.469 mmol) in DMF (2 ml) was added DIPEA (246 μl, 1.407 mmol). The reaction mixture was stirred at RT for 1 h. The crude mixture by C-18 column(100 g cartridge, 0-100% MeCN/Water with 0.05% TFA 0-100% over 16CV) to obtain Tert-butyl (S)-(17-oxo-4,7,10,13-tetraoxa-16-azahenicosa-1,20-diyn-18-yl)carbamate (101 mg, 51% yield). LC/MS: MH⁺=427.5, Rt=0.83 min (3 min acidic run).

### Synthesis of (S)-2-amino-N-(3,6,9,12-tetraoxapentadec-14-yn-1-yl)pent-4-ynamide

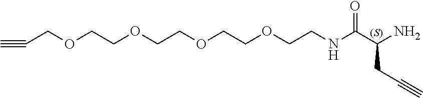

To tert-butyl (S)-(17-oxo-4,7,10,13-tetraoxa-16-azahenicosa-1,20-diyn-18-yl)carbamate (101 mg, 0.237 mmol) was added TFA(25% in DCM) (1460 μl, 4.74 mmol) at 0° C., then raise to RT. The mixture was stirred at rt for 1 h. The mixture was concentrated under high vacuum and then dried under high vacuum overnight to obtain (S)-2-amino-N-(3,6,9,12-tetraoxapentadec-14-yn-1-yl)pent-4-ynamide (TFA salt, 104 mg, 100% yield). LC/MS: MH⁺=327.4, Rt=0.49 min (3 min acidic run).

### Synthesis of tert-butyl (6-oxo-7-(2-(2-(prop-2-yn-1-yloxy)ethoxy)ethyl)-3,10,13-trioxa-7-azahexadec-15-yn-1-yl)carbamate

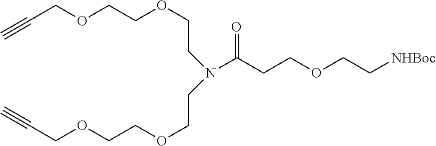 [SMILES: C#CCOCCOCCN(CCOCCOCC#C)C(=O)CCOCCNC(=O)OC(C)(C)C]

To the solution of bis(2-(2-(prop-2-yn-1-yloxy)ethoxy)ethyl)amine (53 mg, 0.197 mmol), 3-(2-((tert-butoxycarbonyl)amino)ethoxy)propanoic acid (48.2 mg, 0.207 mmol) in DMF (1 ml) was added HATU (79 mg, 0.207 mmol) and then DIPEA (103 μl, 0.590 mmol). The reaction mixture was stirred at RT for 1 h. The crude product was purified by RP-C18 ISCO chromatography (elution with MeCN-water, 0.1% formic acid) to give the title product as an oil (81 mg, 85% yield). LC/MS: MH⁺=485.5, Rt=0.92 min (2 min basic method).

### Synthesis of 3-(2-aminoethoxy)-N,N-bis(2-(2-(prop-2-yn-1-yloxy)ethoxy)ethyl)propanamide

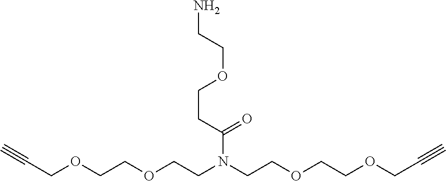 [SMILES: C#CCOCCOCCN(CCOCCOCC#C)C(=O)CCOCCN]

A mixture of tert-butyl (6-oxo-7-(2-(2-(prop-2-yn-1-yloxy)ethoxy)ethyl)-3,10,13-trioxa-7-azahexadec-15-yn-1-yl)carbamate (408 mg, 0.842 mmol) and TFA (1.62 mL, 21.1 mmol) in DCM (4.8 mL) was stirred at RT for 30 min and concentrated. The crude product was purified by RP-C18 ISCO chromatography (50g, gold column)(elution with MeCN-water, 0.1% TFA). Fractions containing the desired product were combined and lyophilized to give the product (TFA salt) as a yellow oil (148 mg, 35% yield). LC/MS: MH⁺=385.3, Rt=0.55 min (2 min acidic method).

### Synthesis of 4,7,13,16,19-pentaoxa-10-azadocos-1-yn-22-oic acid

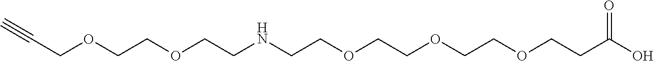 [SMILES: C#CCOCCOCCNCCOCCOCCOCCC(=O)O]

To tert-butyl 10-(tert-butoxycarbonyl)-4,7,13,16,19-pentaoxa-10-azadocos-1-yn-22-oate (250 mg, 0.496 mmol) and triethylsilane (238 μl, 1.489 mmol) in DCM (11.5 ml) at 0° C. was added the TFA (3824 μl, 49.6 mmol) dropwise (TFA/DCM 25% v/v). The mixture was stirred at 0° C. for 15 min, then raised to RT and stirred for 30 min. The crude mixture was concentrated on rotavap under high vacuum, then dry over high vacuum for two days to obtain 4,7,13,16,19-pentaoxa-10-azadocos-1-yn-22-oic acid (231 mg, as crude to use for next reaction without further purification). LC/MS: MH⁺=348.3, Rt=0.47 min (3 min acidic run).

### Synthesis of 2,2-dimethyl-4,11-dioxo-12-(2-(2-(prop-2-yn-1-yloxy)ethoxy)ethyl)-3,8,15,18,21-pentaoxa-5,12-diazatetracosan-24-oic acid

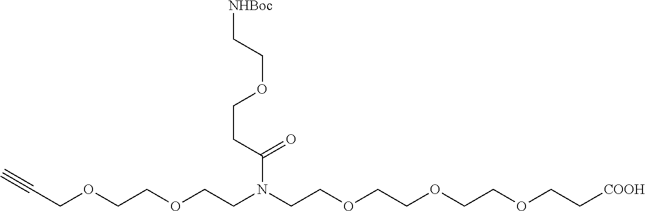 [SMILES: C#CCOCCOCCN(CCOCCOCCOCCC(=O)O)C(=O)CCOCCNC(=O)OC(C)(C)C]

To the solution t-Boc-N-amido-PEG1-acid (139 mg, 0.595 mmol) in DMF (dry, 2 ml) was added HATU (226 mg, 0.595 mmol) and then DIPEA (433 μl, 2.480 mmol). The reaction mixture was stirred at RT for 30 min. Then the solution of 4,7,13,16,19-pentaoxa-10-azadocos-1-yn-22-oic acid (229 mg, 0.496 mmol) in DMF (0.5 ml) was added to the above reaction mixture. The resulting mixture was stirred at RT for 30 min. The crude was separated by C-18 Column (100 g cartridge, eluted with MeCN/Water with 0.05 % TFA, 0-100% over 16 CV) to obtain 2,2-dimethyl-4,11-dioxo-12-(2-(2-(prop-2-yn-1-yloxy)ethoxy)ethyl)-3,8,15,18,21-pentaoxa-5,12-diazatetracosan-24-oic acid (220 mg, 79% yield). LC/MS: MH⁺=563.4, Rt=0.83 min (3 min acidic run).

### Synthesis of allyl (2S,3S,4S,5R,6S)-3,4,5-tris(((allyloxy)carbonyl)oxy)-6-(2-amino-4-(((tert-butyldimethylsilyl)oxy)methyl)phenoxy)tetrahydro-2H-pyran-2-carboxylate

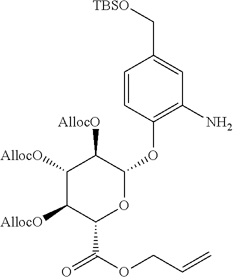

Zn powder (48.7 g, 745 mmol) was added portion wise to a mixture of allyl (2S,3S,4S,5R,6S)-3,4,5-tris(((allyloxy)carbonyl)oxy)-6-(4-(((tert-butyldimethylsilyl)oxy)methyl)-2-nitrophenoxy)tetrahydro-2H-pyran-2-carboxylate (Journal of Natural Products 2015, 78, 510) (56 g, 74.5 mmol), 1,4-dioxane (1.7 mL), H₂O (280 mL), and AcOH (42.6 mL, 745 mmol). The mixture was stirred at 25° C. for 16 h. The mixture was filtered through Celite and washed with 1,4-dioxane (500 mL) and DCM (500 mL). The resulting filtrate was neutralized with saturated NaHCO₃ and extracted with DCM. The combined organic extract was washed with saturated NaHCO₃ and brine. The organic layer was dried and concentrated in vacuo. The residue was purified by NP-column chromatography to give the title product as yellow oil (35.8 g, 66.7% yield). LC/MS: MH⁺=722.2.

Synthesis of allyl (2S,3S,4S,5R,6S)-3,4,5-tris(((allyloxy)carbonyl)oxy)-6-(2-(3-((tert-butoxycarbonyl)amino)propanamido)-4-(((tert-butyldimethylsilyl)oxy)methyl)phenoxy)tetrahydro-2H-pyran-2-carboxylate

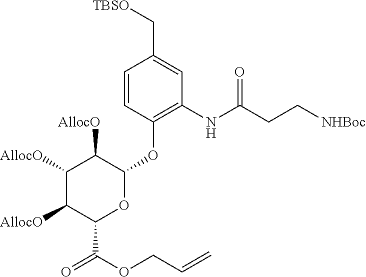

A mixture of allyl (2S,3S,4S,5R,6S)-3,4,5-tris(((allyloxy)carbonyl)oxy)-6-(2-amino-4-(((tert-butyldimethylsilyl)oxy)methyl)phenoxy)tetrahydro-2H-pyran-2-carboxylate (3.00 g, 4.16 mmol), 3-((tert-butoxycarbonyl)amino)propanoic acid (0.95 g, 5.02 mmol), HBTU (1.90 g, 5.01 mmol), and DIPEA (2.22 g, 17.2 mmol) in DMF (15 mL) was stirred at RT for 30 min, and partitioned between EtOAc and brine. The combined organic extract was dried over Na2SO4, concentrated, and chromatographed (NP-ISCO column) to give the title compound (1.66 g, 45% yield). LC/MS: MH⁺=893.7, Rt=1.49 min (2 min acidic method).

### Synthesis of allyl (2S,3S,4S,5R,6S)-3,4,5-tris(((allyloxy)carbonyl)oxy)-6-(2-(3-aminopropanamido)-4-(hydroxymethyl)phenoxy)tetrahydro-2H-pyran-2-carboxylate

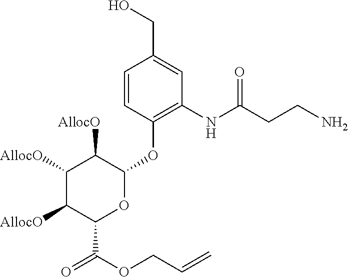

A mixture of allyl (2S,3S,4S,5R,6S)-3,4,5-tris(((allyloxy)carbonyl)oxy)-6-(2-(3-((tert-butoxycarbonyl)amino)propanamido)-4-(((tert-butyldimethylsilyl)oxy)methyl)phenoxy)tetrahydro-2H-pyran-2-carboxylate (1.66 g, 1.86 mmol) and pTsOH (0.036 g, 0.190 mmol) in MeOH (10 mL) was stirred at RT for 10 min. The mixture was concentrated to give the crude O-desilylated product as an oil (LC/MS MH⁺779.6, Rt=1.13 min (2 min acidic method)) which was treated with TFA (6.0 mL, 78 mmol) in DCM (4 mL) at RT for 10 min. The mixture was concentrated to give the crude title product which was used in the next step without purification. LC/MS: MH⁺=679.6, Rt=0.88 min (2 min acidic method).

### Synthesis of allyl (2S,3S,4S,5R,6S)-6-(2-(3-((S)-6-((((9H-fluoren-9-yl)methoxy)carbonyl)amino)-2-((tert-butoxycarbonyl)amino)hexanamido)propanamido)-4-(hydroxymethyl)phenoxy)-3,4,5-tris(((allyloxy)carbonyl)oxy)tetrahydro-2H-pyran-2-carboxylate

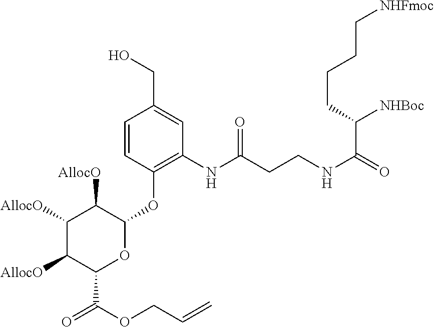

A mixture of allyl (2S,3S,4S,5R,6S)-3,4,5-tris(((allyloxy)carbonyl)oxy)-6-(2-(3-aminopropanamido)-4-(hydroxymethyl)phenoxy)tetrahydro-2H-pyran-2-carboxylate (crude obtained in the previous step), N6-(((9H-fluoren-9-yl)methoxy)carbonyl)-N2-(tert-butoxycarbonyl)-L-lysine (1.2 g, 2.6 mmol), HBTU (1.00 g, 2.64 mmol), and DIPEA (2.00 mL, 11.5 mmol) in DMF (10 mL) was stirred at RT for 10 min. The mixture was partitioned between EtOAc and sequentially 1M HCl and aqueous K₂CO₃. The combined organic extract was dried over Na₂SO₄ and concentrated. The crude product was purified by chromatography (NP-ISCO column, 80 g gold) to give the title product as a white solid (1.11 g, 53% yield over the last three steps).

### Synthesis of allyl (2S,3S,4S,5R,6S)-6-(2-(3-((S)-6-((((9H-fluoren-9-yl)methoxy)carbonyl)amino)-2-((tert-butoxycarbonyl)amino)hexanamido)propanamido)-4-(chloromethyl)phenoxy)-3,4,5-tris(((allyloxy)carbonyl)oxy)tetrahydro-2H-pyran-2-carboxylate

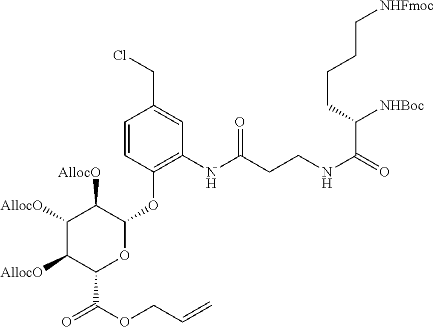

A mixture of allyl (2S,3S,4S,5R,6S)-6-(2-(3-((S)-6-((((9H-fluoren-9-yl)methoxy)carbonyl)amino)-2-((tert-butoxycarbonyl)amino)hexanamido)propanamido)-4-(hydroxymethyl)phenoxy)-3,4,5-tris(((allyloxy)carbonyl)oxy)tetrahydro-2H-pyran-2-carboxylate (1.11 g, 0.983 mmol), thionyl chloride (0.180 mL, 2.466 mmol), and sodium bicarbonate (0.495 g, 5.90 mmol) in THF (10 mL) was stirred at RT for 10 min. The mixture was partitioned between EtOAc and water. The combined organic extract was dried over Na₂SO₄, concentrated, and chromatographed by NP-ISCO column. Fractions containing the desired product were combined and concentrated to give the title product as a white solid (0.95 g, 84% yield). LC/MS: MH⁺=1147.9, Rt=1.39 min (2 min acidic method).

### Synthesis of (S)-9-amino-1-(9H-fluoren-9-yl)-3,10-dioxo-2,14,17-trioxa-4,11-diazaicosan-20-oic acid

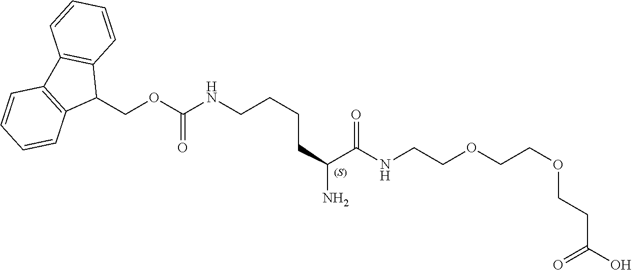

To a clear solution of N6-(((9H-fluoren-9-yl)methoxy)carbonyl)-N2-(tert-butoxycarbonyl)-L-lysine (633 mg, 1.350 mmol) and tert-butyl 3-(2-(2-aminoethoxy)ethoxy)propanoate (300 mg, 1.29 mmol) in Acetonitrile(9 ml) at 0° C. was added DMTMM (340 mg, 1.414 mmol). To the slurry was added DIPEA (0.449 ml, 2.57 mmol). The mixture was stirred at 0° C. for 5 min and then rt for 1.5 h. The solvent was removed by rotary evaporation and under high vac. To the resulting oily material was added CH₂Cl₂ (9 ml), triethylsilane (0.205 ml, 1.286 mmol) and TFA (4.95 ml, 64.3 mmol) at 0° C. The mixture was stirred at rt for 1.5 h. The solvent was removed via rotary evaporation and then under high vac. 1 QThe resulting oil was dissolved in DMSO/water and was purified by RP-HPLC (C18, ACN/water with 0.1% TFA) to afford, after lyophilization, a white powder as (S)-9-amino-1-(9H-fluoren-9-yl)-3,10-dioxo-2,14,17-trioxa-4,11-diazaicosan-20-oic acid as a TFA salt (754 mg, 91% yield). LC/MS: MH⁺=528.3, rt=0.79 min (2 min acidic method). ¹H NMR (400 MHz, DMSO-d6) δ 8.48 (t, J=5.6 Hz, 1H), 8.24-8.00 (m, 3H), 7.90 (d, J=7.5 Hz, 2H), 7.68 (d, J=7.5 Hz, 2H), 7.47-7.37 (m, 2H), 7.37-7.29 (m, 2H), 7.29-7.19 (m, 1H), 4.31 (d, J=6.8 Hz, 2H), 4.21 (t, J=6.8 Hz, 1H), 3.94-3.65 (m, 1H), 3.59 (dd, J=7.5, 5.2 Hz, 2H), 3.45 (dd, J=13.3, 7.7 Hz, 3H), 3.39-3.14 (m, 2H), 3.01-2.89 (m, 2H), 2.44 (t, J=6.3 Hz, 2H), 1.81-1.59 (m, 2H), 1.46-1.15 (m, 4H).

### Synthesis of (S)-12-(4-((((9H-fluoren-9-yl)methoxy)carbonyl)amino)butyl)-11,14-dioxo-4,7,17,20-tetraoxa-10,13-diazatricos-22-ynoic acid

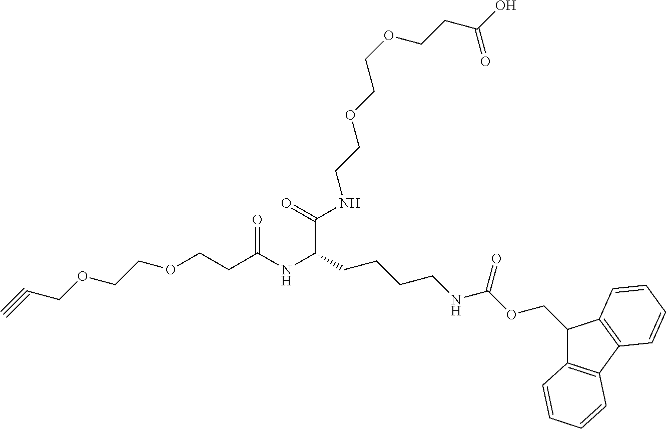 [SMILES: C#CCOCCOCCC(=O)N[C@@H](CCCCNC(=O)OCC1c2ccccc2-c2ccccc21)C(=O)NCCOCCOCCC(=O)O]

To 3-(2-(prop-2-yn-1-yloxy)ethoxy)propanoic acid (65.5 mg, 0.381 mmol) in DMF (2 ml) was added DIPEA (0.240 ml, 1.37 mmol) and HATU (150 mg, 0.394 mmol). The mixture was stirred at rt for 15 min. Then at 0° C. (S)-9-amino-1-(9H-fluoren-9-yl)-3,10-dioxo-2,14,17-trioxa-4,11-diazaicosan-20-oic acid as a TFA salt (220 mg, 0.343 mmol) in DMF (2 ml) was added. The mixture was stirred at rt for 1 h and was diluted with DMSO/water (10/4 mL) and purified by RP-HPLC (C18, ACN/water with 0.1% TFA) to afford, after lyophilization, a white floppy powder as (S)-12-(4-((((9H-fluoren-9-yl)methoxy)carbonyl)amino)butyl)-11,14-dioxo-4,7,17,20-tetraoxa-10,13-diazatricos-22-ynoic acid (117 mg, 50% yield). LC/MS: MH⁺=682.4, Rt=0.97 min (2 min acidic method); HRMS: MH⁺=682.2300, Rt=2.16 min (5 min acidic method).

### Synthesis of (S)-9-amino-1-(9H-fluoren-9-yl)-3,10-dioxo-2,14,17,20-tetraoxa-4,11-diazatricosan-23-oic acid

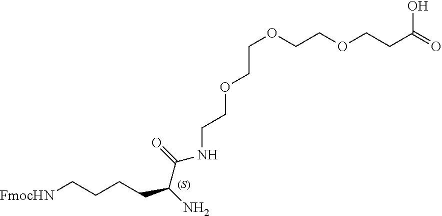

N6-(((9H-fluoren-9-yl)methoxy)carbonyl)-N2-(tert-butoxycarbonyl)-L-lysine (785 mg, 1.68 mmol) and tert-butyl 3-(2-(2-(2-aminoethoxy)ethoxy)ethoxy)propanoate in Acetonitrile (Volume: 12 ml) at 0° C. was DMTMM (422 mg, 1.76 mmol). To the slurry was added DIPEA (0.558 ml, 3.19 mmol). The mixture was stirred at 0° C. for 5 min and then rt for 1.5 h. The solvent was removed by rotary evaporation and under high vac. To the resulting oily material was added CH₂Cl₂ (12 ml), triethylsilane (0.255 ml, 1.60 mmol) and TFA (6.15 ml, 80 mmol) at 0° C. The mixture was stirred at rt for 1.5 h. The solvent was removed via rotary evaporation and then under high vac.D D The resulting oil was dissolved in DMSO/water and was purified by RP-HPLC (C18, ACN/water with 0.1% TFA) to afford, after lyophilization, a white powder as (S)-9-amino-1-(9H-fluoren-9-yl)-3,10-dioxo-2,14,17,20-tetraoxa-4,11-diazatricosan-23-oic acid as a TFA salt (933 mg, 85% yield). LC/MS: MH⁺=572.4, Rt=0.79 min (acidic, 2 min acidic method). ¹H NMR (400 MHz, DMSO-d6) δ 8.54-8.45 (m, 1H), 8.17 (s, 1H), 8.06 (s, 2H), 7.90 (dt, J=7.6, 0.9 Hz, 2H), 7.68 (d, J=7.5 Hz, 2H), 7.46-7.39 (m, 2H), 7.38-7.31 (m, 2H), 7.30-7.18 (m, 1H), 4.31 (d, J=6.8 Hz, 2H), 4.21 (t, J=6.8 Hz, 1H), 3.94-3.65 (m, 1H), 3.59 (t, J=6.3 Hz, 2H), 3.44 (t, J=5.5 Hz, 8H), 3.38-3.18 (m, 2H), 2.96 (q, J=6.6 Hz, 2H), 2.61 (s, 2H), 2.62-2.52 (m, 2H), 2.51 (s, 2H), 2.43 (t, J=6.3 Hz, 2H), 1.82-1.61 (m, 2H), 1.46-1.15 (m, 4H).

### Synthesis of (S)-15-(4-((((9H-fluoren-9-yl)methoxy)carbonyl)amino)butyl)-14,17-dioxo-4,7,10,20,23-pentaoxa-13,16-diazahexacos-25-ynoic acid

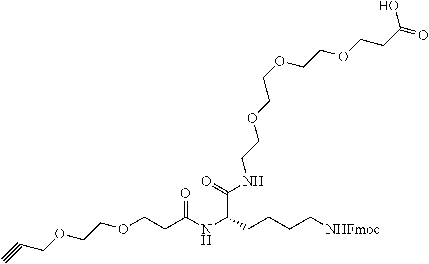

To 3-(2-(prop-2-yn-1-yloxy)ethoxy)propanoic acid (84 mg, 0.49 mmol) in DMF (2.5 ml) was added DIPEA (0.309 ml, 1.77 mmol) and HATU (193 mg, 0.508 mmol). The mixture was stirred at rt for 15 min. Then at 0° C. (S)-9-amino-1-(9H-fluoren-9-yl)-3,10-dioxo-2,14,17,20-tetraoxa-4,11-diazatricosan-23-oic acid as a TFA salt (303 mg, 0.442 mmol) in DMF (2.5 ml) was added. The mixture was stirred at rt for 1 h and was diluted with DMSO/water (10/4 mL) and purified by RP-HPLC (C18, ACN/water with 0.1% TFA) to afford, after lyophilization, a white floppy powder as (S)-15-(4-((((9H-fluoren-9-yl)methoxy)carbonyl)amino)butyl)-14,17-dioxo-4,7,10,20,23-pentaoxa-13,16-diazahexacos-25-ynoic acid (206 mg, 64% yield). HRMS: MH⁺=726.3700, Rt=2.19 min (5 min acidic method).

### Synthesis of tert-butyl 1-(9H-fluoren-9-yl)-11-(2-hydroxyethyl)-3,10-dioxo-2,7,14,17,20-pentaoxa-4,11-diazatricosan-23-oate

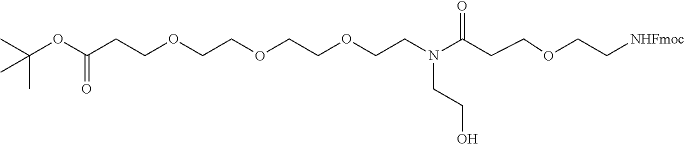

In a 10 mL glass vial equipped with magnetic stir bar, to tert-butyl 3-(2-(2-(2-(tosyloxy)ethoxy)ethoxy)ethoxy)propanoate (311 mg, 0.719 mmol) dissolved in EtOH (1 mL) was added Ethanoamine (220 mg, 3.60 mmol). The reaction mixture was stirred at 75° C. for 2 h, cooled to RT. The solvent was removed under reduced pressure and dried under high vac overnight to afford a thick oil (515 mg). To this thick oil (412 mg) was added at 0° C. 3-(2-((((9H-fluoren-9-yl)methoxy)carbonyl)amino)ethoxy)propanoic acid (1329 mg, 3.74 mmol) in Acetonitrile (6 mL) and THF (3 mL), DMTMM (1070 mg, 3.86 mmol) and DIPEA (0.800 mL, 4.60 mmol). The mixture was stirred at rt for 1 h. The cloudy mixture was filtered. The filtrate was concentrated and purified by flash chromatography (0-10% MeOH in CH₂Cl₂ elution) twice to provide, after concentration of appropriate fractions, tert-butyl 1-(9H-fluoren-9-yl)-11-(2-hydroxyethyl)-3,10-dioxo-2,7,14,17,20-pentaoxa-4,11-diazatricosan-23-oate as a white solid (194 mg, yield 51.5%). LC/MS: MH⁺=659.5, Rt=1.13 min (2 min acidic method). ¹H NMR (400 MHz, DMSO-d6) δ 7.89 (d, J=7.5 Hz, 2H), 7.70 (d, J=7.4 Hz, 2H), 7.45 -7.38 (m, 2H), 7.37-7.25 (m, 3H), 4.69 (d, J=64.3 Hz, 1H), 4.41-4.01 (m, 4H), 3.88 (d, J=33.5 Hz, 3H), 3.64-3.55 (m, 4H), 3.54-3.49 (m, 4H), 3.46-3.28 (m, 10H), 3.20-3.08 (m, 5H), 2.64-2.56 (m, 2H), 2.44-2.37 (m, 2H), 1.39 (d, J=1.9 Hz, 9H).

### Synthesis of 11-(2-(((N-(2-(2-(2-carboxyethoxy)ethoxy)ethyl)sulfamoyl)carbamoyl)oxy)ethyl)-1-(9H-fluoren-9-yl)-3,10-dioxo-2,7,14,17,20-pentaoxa-4,11-diazatricosan-23-oic acid

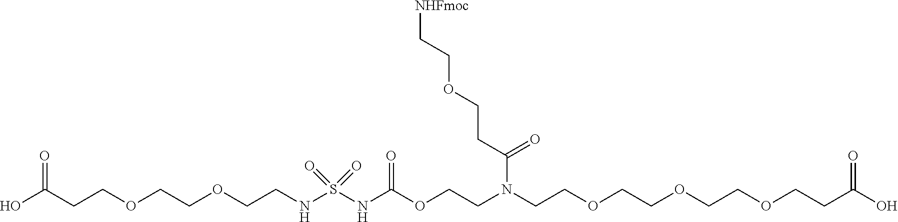

To tert-butyl 1-(9H-fluoren-9-yl)-11-(2-hydroxyethyl)-3,10-dioxo-2,7,14,17,20-pentaoxa-4,11-diazatricosan-23-oate (97 mg, 0.147 mmol) in CH₂Cl₂ (2.5 ml) was added sulfurisocyanatidic chloride (0.013 ml, 0.15 mmol) at 0° C. The mixture was stirring at 0° C. for 30 min. Then TEA (0.103 ml, 0.736 mmol) and tert-butyl 3-(2-(2-aminoethoxy)ethoxy)propanoate (37.8 mg, 0.162 mmol) in CH₂Cl₂ (1.2 mL) were added. The mixture was stirred at 0° C. for 1 h, then rt for 1 h and was quenched with satd. NH₄C1, and 1 N HCl (0.9 mL). The aqueous was extracted with CH₂Cl₂ (5X). The organic layer was dried over anh, Na₂SO₄, filtered and concentrated via rotary evaporation to provide a clear oil. Purification was conducted by flash chromatography (0-100% EtOAc in heptane, then 0-15% MeOH in CH₂Cl₂) to afford the di-t-butyl ester of the title compound as a clear oil (25 mg, 17% yield). LC/MS: MH⁺=997.6, Rt=1.38 min. HR/MS (peptide, 5 min): 3.01 min, M⁺=997.4900, 100% pure. ¹H NMR (400 MHz, Methylene Chloride-d2) δ 7.86-7.78 (m, 2H), 7.69 (t, J=7.9 Hz, 2H), 7.49-7.40 (m, 2H), 7.40-7.31 (m, 2H), 6.41-6.13 (m, 1H), 6.04-5.50 (m, 1H), 4.50-4.21 (m, 5H), 3.92-3.66 (m, 9H), 3.66-3.49 (m, 20H), 3.44-3.22 (m, 4H), 2.55-2.45 (m, 4H), 1.47 (s, 18H). To the di-ester in CH₂Cl₂ (1.3 ml) at 0° C. was added triethylsilane (0.004 ml, 0.03 mmol) and TFA (0.567 ml, 7.36 mmol). The mixture was stirred at rt for 1 h and then concentrated via rotary evaporation. The residue was dried in vacuo for 30 min and then lyophilized (water/ACN=3/3 mL) to afford 11-(2-(((N-(2-(2-(2-carboxyethoxy)ethoxy)ethyl)sulfamoyl)carbamoyl)oxy)ethyl)-1-(9H-fluoren-9-yl)-3,10-dioxo-2,7,14,17,20-pentaoxa-4,11-diazatricosan-23-oic acid as a white solid (24 mg). HRMS: MH⁺=885.3500, Rt=2.05 min (5 min acidic method). ¹H NMR (400 MHz, DMSO) δ 12.12 (s, 2H), 7.89 (d, J=7.5 Hz, 2H), 7.69 (d, J=7.5 Hz, 2H), 7.46-7.39 (m, 2H), 7.37-7.30 (m, 2H), 7.28 (t, J=5.2 Hz, 1H), 4.33-4.08 (m, 5H), 3.67-3.55 (m, 7H), 3.55-3.41 (m, 20H), 3.41-3.36 (m, 2H), 3.18-2.99 (m, 4H), 2.69-2.56 (m, 2H), 2.47-2.39 (m, 5H).

### Synthesis of benzyl 3-(2-(2-(2-aminoethoxy)ethoxy)ethoxy)propanoate

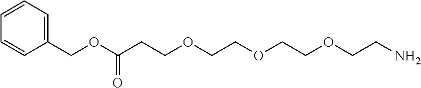 [SMILES: NCCOCCOCCOCCC(=O)OCc1ccccc1]

To benzyl 2,2-dimethyl-4-oxo-3,8,11,14-tetraoxa-5-azaheptadecan-17-oate (1000 mg, 2.430 mmol) in CH₂Cl₂ (24 mL) was added triethylsilane (0.388 mL, 2.43 mmol) and TFA (8 mL, 0.1 mol). The mixture was stirred at rt for 1 h. The volatile was removed via rotary evaporation and under high vac for 15 min, then diluted with ACN/water (5/5 mL) and lyophilized to give benzyl 3-(2-(2-(2-aminoethoxy)ethoxy)ethoxy)propanoate as a TFA salt form as a colorless oil (1.269 g). LC/MS: MH⁺=312.3, Rt=0.61 min (2 min acidic method). ¹H NMR (400 MHz, DMSO) δ 7.74 (s, 3H), 7.43-7.28 (m, 5H), 5.11 (s, 2H), 3.66 (t, J=6.2 Hz, 2H), 3.60-3.54 (m, 6H), 3.50 (s, 4H), 2.97 (h, J=5.7 Hz, 2H), 2.61 (t, J=6.2 Hz, 2H).

### Synthesis of benzyl (S)-10-amino-2,2-dimethyl-4,11-dioxo-3,15,18,21-tetraoxa-5,12-diazatetracosan-24-oate

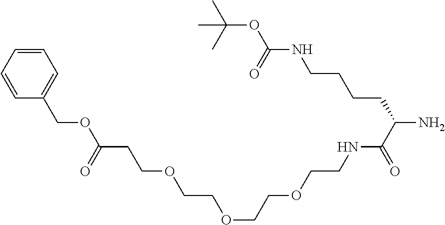 [SMILES: CC(C)(C)OC(=O)NCCCC[C@H](N)C(=O)NCCOCCOCCOCCC(=O)OCc1ccccc1]

To a mixture of benzyl 3-(2-(2-(2-aminoethoxy)ethoxy)ethoxy)propanoate as a TFA salt (707 mg, 1.66 mmol) and Fmoc-Lys(Boc)-OSu (987 mg, 1.75 mmol) in DMF (6 mL) was added DIPEA (0.871 mL, 4.99 mmol). The mixture was stirred at rt overnight. To the mixture was added dimethylamine (2N in THF, 4.15 mL, 8.31 mmol). The mixture was stirred at rt for 2 h, diluted with DMSO (10 mL) and purified by RP-HPLC (C18, ACN/water with 0.1% TFA) to afford, after lyophilization of the appropriate fractions, benzyl (S)-10-amino-2,2-dimethyl-4,11-dioxo-3,15,18,21-tetraoxa-5,12-diazatetracosan-24-oate as a TFA salt form as a clear thick oil (630 mg, 58% yield). LC/MS: MH⁺=540.5, Rt=0.83 min (2 min acidic method). HRMS: MH⁺=540.3300, Rt=1.74 min (5 min acidic method). ¹H NMR (400 MHz, DMSO) δ 8.48 (t, J=5.6 Hz, 1H), 8.05 (d, J=4.8 Hz, 3H), 7.43-7.26 (m, 5H), 6.76 (t, J=5.7 Hz, 1H), 5.11 (s, 2H), 3.73-3.59 (m, 5H), 3.48-3.17 (m, 10H), 2.93-2.84 (m, 2H), 2.61 (t, J=6.2 Hz, 2H), 1.71 -1.61 (m, 2H), 1.37 (s, 11H), 1.31-1.19 (m, 2H).

### Synthesis of 1-benzyl 29-methyl (S)-15-(4-((tert-butoxycarbonyl)amino)butyl)-14,17-dioxo-4,7,10,20,23,26-hexaoxa-13,16-diazanonacosanedioate

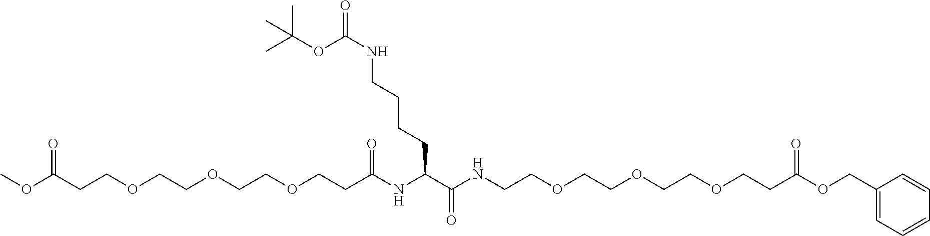 [SMILES: COC(=O)CCOCCOCCOCCC(=O)N[C@@H](CCCCNC(=O)OC(C)(C)C)C(=O)NCCOCCOCCOCCC(=O)OCc1ccccc1]

To benzyl (S)-10-amino-2,2-dimethyl-4,11-dioxo-3,15,18,21-tetraoxa-5,12-diazatetracosan-24-oate as a TFA salt form (630 mg, 1.17 mmol) and 3-oxo-2,6,9,12-tetraoxapentadecan-15-oic acid (324 mg, 1.226 mmol) in Acetonitrile (8 ml) at) at 0° C. was added DMTMM (303 mg, 1.26 mmol) and DIPEA (0.816 ml, 4.67 mmol). The mixture was stirred at for 1.5 h. The volatile was removed via rotary evaporation and then in high vac for 15 min. The resulting colorless oil was purified by flash chromatography (0-10% MeOH in CH₂Cl₂) to provide 1-benzyl 29-methyl (S)-15-(4-((tert-butoxycarbonyl)amino)butyl)-14,17-dioxo-4,7,10,20,23,26-hexaoxa-13,16-diazanonacosanedioate as a colorless oil (551 mg, yield 61%). LC/MS: MH⁺=786.4, Rt=1.02 min (2 min acidic method). ¹H NMR (400 MHz, DMSO) δ 7.94-7.85 (m, 2H), 7.41-7.28 (m, 5H), 6.72 (t, J=5.7 Hz, 1H), 5.11 (s, 2H), 4.23 -4.11 (m, 1H), 3.70-3.55 (m, 9H), 3.48 (t, J=3.0 Hz, 16H), 3.38 (t, J=6.0 Hz, 2H), 3.26-3.10 (m, 2H), 2.91-2.81 (m, 2H), 2.58 (dd, J=26.8, 6.2 Hz, 4H), 2.45-2.29 (m, 2H), 1.37 (m, 15H).

### Synthesis of (S)-17-(4-((tert-butoxycarbonyl)amino)butyl)-3,15,18-trioxo-2,6,9,12,22,25,28-heptaoxa-16,19-diazahentriacontan-31-oic acid

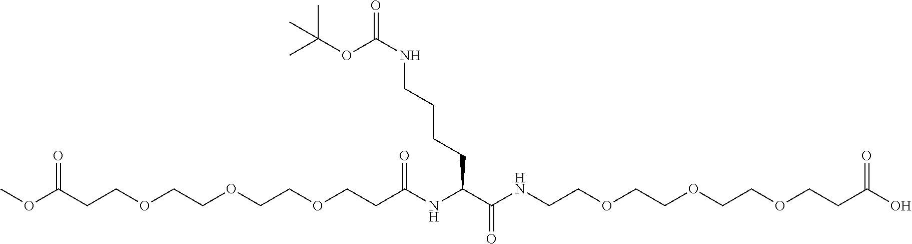 [SMILES: COC(=O)CCOCCOCCOCCC(=O)N[C@@H](CCCCNC(=O)OC(C)(C)C)C(=O)NCCOCCOCCOCCC(=O)O]

A mixture of 1-benzyl 29-methyl (S)-15-(4-((tert-butoxycarbonyl)amino)butyl)-14,17-dioxo-4,7,10,20,23,26-hexaoxa-13,16-diazanonacosanedioate (202 mg, 0.257 mmol) and Pd-C(10% on carbon, 27 mg, 0.026 mmol) in THF (10 mL) was purged with H₂ three times and then stirred vigorously under balloon hydrogen for 3.5 h. The mixture was filtered and washed first with CH₂Cl₂ and then with 1:1 CH₂Cl₂/MeOH (20 mL). The filtrate was concentrated via rotary evaporation and then under high vac overnight to afford (S)-17-(4-((tert-butoxycarbonyl)amino)butyl)-3,15,18-trioxo-2,6,9,12,22,25,28-heptaoxa-16,19-diazahentriacontan-31-oic acid as a thick oil (180 mg, 100% yield). LC/MS: MH⁺=696.3, Rt=0.79 min (2 min acidic method). LC/MS: MH⁺=696.8, Rt=1.64 min (5 min acidic method). ¹H NMR (400 MHz, DMSO) δ 12.13 (s, 1H), 7.90 (q, J=7.0 Hz, 2H), 6.72 (d, J=5.7 Hz, 1H), 4.26-4.11 (m, 1H), 3.66-3.54 (m, 9H), 3.54-3.42 (m, 16H), 3.39 (t, J=5.9 Hz, 2H), 3.26-3.11 (m, 2H), 2.91-2.81 (m, 2H), 2.57-2.52 (m, 2H), 2.47-2.28 (m, 4H), 1.63-1.14 (m, 15H).

### Synthesis of benzyl 3-(2-(2-aminoethoxy)ethoxy)propanoate

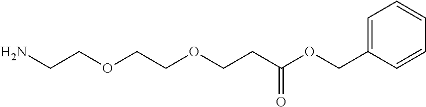 [SMILES: NCCOCCOCCC(=O)OCc1ccccc1]

To benzyl 2,2-dimethyl-4-oxo-3,8,11-trioxa-5-azatetradecan-14-oate (1110 mg, 3.02 mmol) in CH₂Cl₂ (24 mL) was added triethylsilane (0.483 mL, 3.02 mmol) and TFA (8 mL, 0.1 mol). The mixture was stirred at rt for 1 h. The volatile was removed via rotary evaporation and under high vac for 15 min, then diluted with ACN/water (5/5 mL) and lyophilized to give benzyl 3-(2-(2-aminoethoxy)ethoxy)propanoate as a TFA salt form as a light-yellow oil (1.132 g, yield 98%). LC/MS: MH⁺=268.2, Rt=0.58 min (2 min acidic method).

### Synthesis of benzyl (S)-10-amino-2,2-dimethyl-4,11-dioxo-3,15,18-trioxa-5,12-diazahenicosan-21-oate

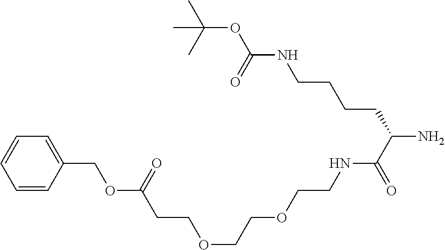 [SMILES: CC(C)(C)OC(=O)NCCCC[C@H](N)C(=O)NCCOCCOCCC(=O)OCc1ccccc1]

To a benzyl 3-(2-(2-(2-aminoethoxy)ethoxy)ethoxy)propanoate as a TFA salt form (1132 mg, 2.97 mmol) and Fmoc-Lys(Boc)-OSu (1763 mg, 3.12 mmol) in DMF (10 mL) was added DIPEA (1.56 ml, 8.91 mmol). The mixture was stirred at rt overnight. To the mixture was added dimethylamine (2N in THF, 7.42 ml, 14.8 mmol). The mixture was stirred at rt for 2 h, diluted with DMSO (15 mL) and purified by RP-HPLC (C18, ACN/water with 0.1% TFA) to afford, after lyophilization of the appropriate fractions, benzyl (S)-10-amino-2,2-dimethyl-4,11-dioxo-3,15,18-trioxa-5,12-diazahenicosan-21-oate as a TFA salt form as a clear thick oil (947 mg, yield 52%). LC/MS: MH⁺=496.5, Rt=0.81 min (2 min acidic method). ¹H NMR (400 MHz, DMSO) δ 8.48 (t, J=5.6 Hz, 1H), 8.12-8.00 (m, 3H), 7.41-7.29 (m, 5H), 6.76 (t, J=5.7 Hz, 1H), 5.11 (s, 2H), 3.75-3.63 (m, 4H), 3.51 (s, 3H), 3.44 (t, J=5.6 Hz, 2H), 3.38-3.17 (m, 2H), 2.94-2.82 (m, 2H), 2.61 (t, J=6.2 Hz, 2H), 1.71-1.61 (m, 2H), 1.37 (m, 13H).

### Synthesis of 1-benzyl 29-methyl (S)-12-(4-((tert-butoxycarbonyl)amino)butyl)-11,14-dioxo-4,7,17,20,23,26-hexaoxa-10,13-diazanonacosanedioate

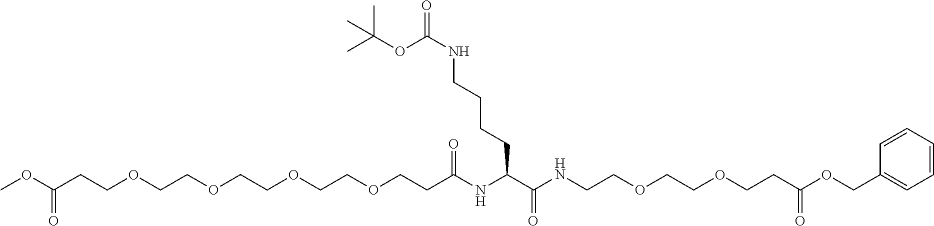 [SMILES: COC(=O)CCOCCOCCOCCOCCC(=O)N[C@@H](CCCCNC(=O)OC(C)(C)C)C(=O)NCCOCCOCCC(=O)OCc1ccccc1]

To benzyl (S)-10-amino-2,2-dimethyl-4,11-dioxo-3,15,18-trioxa-5,12-diazahenicosan-21-oate as a TFA salt form (1043 mg, 1.71 mmol) and 3-oxo-2,6,9,12,15-pentaoxaoctadecan-18-oic acid (501 mg, 1.624 mmol).in Acetonitrile (10 ml) at) at 0° C. was added DMTMM (401 mg, 1.67 mmol) and DIPEA (1.081 ml, 6.19 mmol). The mixture was stirred at rt for 2.5 h. To the mixture were added 3-oxo-2,6,9,12,15-pentaoxaoctadecan-18-oic acid (150 mg, 0.487 mmol) and DMTMM (120 mg, 0.501 mmol) and DIPEA (0.324 ml, 1.86 mmol). The mixture was stirred at rt for 30 min. The volatile was removed via rotary evaporation and then in high vac for 15 min. The resulting colorless oil was purified by flash chromatography (0-10% MeOH in CH₂Cl₂), followed by washing of the product thereof with EtOAc with 1:1 satd. NaHCO₃/H₂O, to provide 1-benzyl 29-methyl (S)-12-(4-((tert-butoxycarbonyl)amino)butyl)-11,14-dioxo-4,7,17,20,23,26-hexaoxa-10,13-diazanonacosanedioate as a white solid (1062 mg, yield 79%). LC/MS: MH⁺=786.7, Rt=1.04 min (2 min acidic method). ¹H NMR (400 MHz, DMSO) δ 7.95-7.84 (m, 2H), 7.42-7.29 (m, 5H), 6.72 (t, J=5.7 Hz, 1H), 5.11 (s, 2H), 4.25-4.14 (m, 1H), 3.70-3.55 (m, 9H), 3.53-3.44 (m, 16H), 3.38 (t, J=6.0 Hz, 2H), 3.25-3.13 (m, 2H), 2.91-2.81 (m, 2H), 2.58 (dd, J=26.1, 6.2 Hz, 4H), 2.45-2.29 (m, 2H), 1.37 (m, 16H).

### Synthesis of (S)-20-(4-((tert-butoxycarbonyl)amino)butyl)-3,18,21-trioxo-2,6,9,12,15,25,28-heptaoxa-19,22-diazahentriacontan-31-oic acid

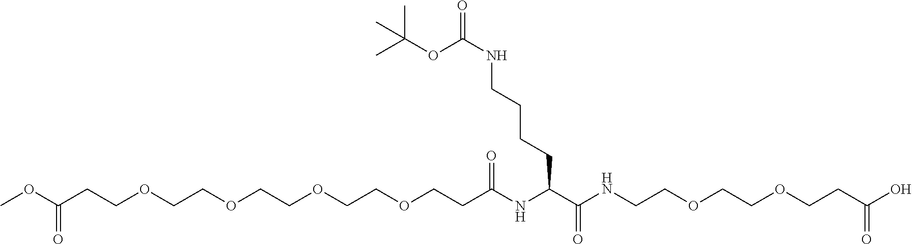 [SMILES: COC(=O)CCOCCOCCOCCOCCC(=O)N[C@@H](CCCCNC(=O)OC(C)(C)C)C(=O)NCCOCCOCCC(=O)O]

A mixture of 1-benzyl 29-methyl (S)-12-(4-((tert-butoxycarbonyl)amino)butyl)-11,14-dioxo-4,7,17,20,23,26-hexaoxa-10,13-diazanonacosanedioate (620 mg, 789 μmol) and Pd-C(10% on carbon, 84 mg, 0.079 mmol) in THF (30 mL) was purged with H₂ three times and then stirred vigorously under balloon hydrogen for 3.5 h. The mixture was filtered and washed first with CH₂Cl₂ and then with 1:1 CH₂Cl₂/MeOH (60 mL). The filtrate was concentrated via rotary evaporation and then under high vac overnight to afford (S)-20-(4-((tert-butoxycarbonyl)amino)butyl)-3,18,21-trioxo-2,6,9,12,15,25,28-heptaoxa-19,22-diazahentriacontan-31-oic acid (620 mg, 789 μmol). LC/MS: MH⁺=696.7, Rt=1.08 min (2 min basic method). ¹H NMR (400 MHz, DMSO) δ 12.14 (s, 1H), 7.96-7.82 (m, 2H), 6.73 (t, J=5.6 Hz, 1H), 4.26-4.14 (m, 1H), 3.67-3.54 (m, 9H), 3.52-3.44 (m, 15H), 3.39 (t, J=6.0 Hz, 2H), 3.27 -3.10 (m, 2H), 2.92-2.81 (m, 2H), 2.55 (d, J=6.2 Hz, 3H), 2.47-2.34 (m, 4H), 1.37 (m, 15H).

### Synthesis of tert-butyl (2-(3-oxo-3-((2-(2,2,2-trifluoroacetamido)ethyl)(2-(tritylamino)ethyl)amino)propoxy)ethyl)carbamate

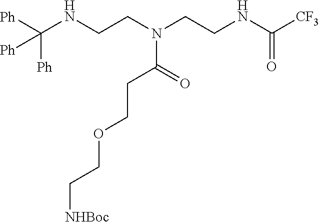 [SMILES: CC(C)(C)OC(=O)NCCOCCC(=O)N(CCNC(=O)C(F)(F)F)CCNC(c1ccccc1)(c1ccccc1)c1ccccc1]

To 2,2,2-trifluoro-N-(2-((2-(tritylamino)ethyl)amino)ethyl)acetamide (14.061 g, 31.85 mmol, J. Org. Chem. 2012, 77, 4226-4234) and 3-(2-((tert-butoxycarbonyl)amino)ethoxy)propanoic acid (7.949 g, 34.08 mmol) in CH₂Cl₂ (600 mL) at 0° C. was added in 5 potions over 20 min EDC (7.326 g, 38.22 mmol). The clear solution was stirred at 0° C. for 30 min, then warmed up to rt overnight. To the mixture was added, more 3-(2-((tert-butoxycarbonyl)amino)ethoxy)propanoic acid (0.556 g, 2.38 mmol) and EDC (0.733 g, 3.82 mmol). The mixture was stirred at rt for 4 h, and was concentrated to ca. 400 mL and was washed with 1:1 brine/water (150 mL). The aqueous was extracted with CH₂Cl₂ (20 mL). The washing and back extraction procedure was repeated three more times. The combined organic phase was washed with satd. NaHCO₃ (50 mL×2), brine, dried over anh. Na₂SO₄, filtered and concentrated to provide tert-butyl (2-(3-oxo-3-((2-(2,2,2-trifluoroacetamido)ethyl)(2-(tritylamino)ethyl)amino)propoxy)ethyl)carbamate as a white foam (19.98 g, yield 90%). LC/MS: M⁺Na⁺=679.5, M-=655.5, Rt=1.18 min. (2 min acidic). The product was used directly in the next step without purification.

### Synthesis of tert-butyl (2-(3-((2-aminoethyl)(2-(tritylamino)ethyl)amino)-3-oxopropoxy)ethyl)carbamate

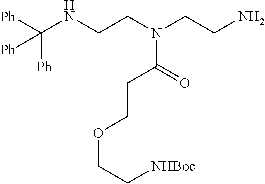 [SMILES: CC(C)(C)OC(=O)NCCOCCC(=O)N(CCN)CCNC(c1ccccc1)(c1ccccc1)c1ccccc1]

To tert-butyl (2-(3-oxo-3-((2-(2,2,2-trifluoroacetamido)ethyl)(2-(tritylamino)ethyl)amino)propoxy)ethyl)carbamate (9.210, 14.02 mmol) dissolved in MeOH (130 mL) and water (13 mL) was added K₂CO₃ (9.69 g, 70.1 mmol). The slurry was stirred at rt over weekend and then was filtered and washed with EtOAc. The filtrate was concentrated. The residue was washed with 1:1 brine/water (80 mL). The aqueous was extracted with EtOAc (3X). The combined organic layer was washed with brine, dried over anh. Na₂SO₄, filtered and concentrated. To remove the residual solvent, the product was dissolved with CH₂Cl₂ and concentrated. This was repeated one more time to afford tert-butyl (2-(3-((2-aminoethyl)(2-(tritylamino)ethyl)amino)-3-oxopropoxy)ethyl)carbamate, following drying under high vac, as a white foaming (7.26 g, 92% yield). LC/MS: MH⁺=561.6, Rt=2.19 min. (5 min acidic method). ¹H NMR (400 MHz, DMSO) δ 7.45-7.35 (m, 6H), 7.33-7.25 (m, 6H), 7.24-7.13 (m, 3H), 6.72 (q, J=6.3 Hz, 1H), 3.57 (m, J=13.0, 6.6 Hz, 2H), 3.43-3.25 (m, 11H, overlapping with water), 3.22-2.94 (m, 4H), 2.80-2.53 (m, 4H), 2.47-2.23 (m, 2H), 2.18-2.02 (m, 2H), 1.37 (d, J=1.3 Hz, 9H). The product was used directly in the next step without purification.

### Synthesis of 3-(2-(3-(allyloxy)-3-oxopropoxy)ethoxy)propanoic acid

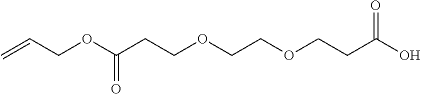 [SMILES: C=CCOC(=O)CCOCCOCCC(=O)O]

To a mixture of 3,3′-(ethane-1,2-diylbis(oxy))dipropionic acid (7.720 g, 37.44 mmol), prop-2-en-1-ol (2.175 g, 2.55 mL, 37.4 mmol) and DMAP (457 mg, 3.74 mmol) in CH₂Cl₂ (66 mL) and dioxane (100.0 mL) at 0° C. was added dropwise, dicyclohexylmethanediimine (8.112 g, 39.31 mmol) slurry in dichloromethane (34 mL) over 20 min. The mixture was warmed up to rt and stirred overnight. The reaction mixture was filtered and the solid was washed with CH₂Cl₂ (100 mL). The volatile from the filtrate was removed under reduced pressure. The resulting oil was mixed with DMSO (24 mL) and filtered. The filtrate was purified by RP-HPLC (C18, ACN/water with 0.1% TFA), to provide, after lyophilization of the appropriate fractions, 3-(2-(3-(allyloxy)-3-oxopropoxy)ethoxy)propanoic acid as a colorless oil (4.487 g, 49% yield). LC/MS: MH⁺=247.0, Rt=0.66 min (2 min acidic method) ¹H NMR (400 MHz, DMSO) δ 5.97-5.84 (m, 1H), 5.34-5.26 (m, 1H), 5.24-5.17 (m, 1H), 4.59-4.54 (m, 2H), 3.67-3.56 (m, 4H), 3.52-3.30 (m, 6H, overlapping with DMSO), 2.57 (t, J=6.2 Hz, 2H), 2.43 (t, J=6.4 Hz, 2H).

### Synthesis of allyl 2,2-dimethyl-4,11,16-trioxo-12-(2-(tritylamino)ethyl)-3,8,19,22-tetraoxa-5,12,15-triazapentacosan-25-oate

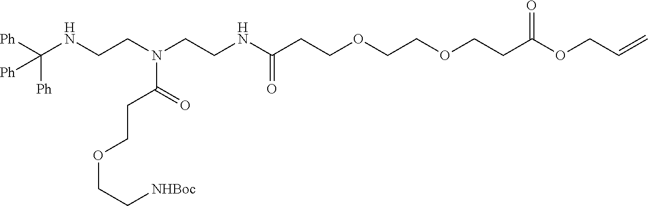 [SMILES: C=CCOC(=O)CCOCCOCCC(=O)NCCN(CCNC(c1ccccc1)(c1ccccc1)c1ccccc1)C(=O)CCOCCNC(=O)OC(C)(C)C]

To tert-butyl (2-(3-((2-aminoethyl)(2-(tritylamino)ethyl)amino)-3-oxopropoxy)ethyl)carbamate (8420 mg, 15.02 mmol) and 3-(2-(3-(allyloxy)-3-oxopropoxy)ethoxy)propanoic acid (3.957 g,16.07 mmol) dissolved in CH₂Cl₂ (150 mL) at 0° C. was added EDC (3.224 g, 16.82 mmol) in two portions over 10 min. The mixture was warmed up and stirred at rt overnight. To the mixture was added 3-(2-(3-(allyloxy)-3-oxopropoxy)ethoxy)propanoic acid (277 mg, 1.12 mmol), and EDC (226 mg, 1.18 mmol). After being stirred at rt overnight, the mixture was diluted with CH₂Cl₂ (200 mL) and was washed with 1:1 brine/water (200 mL). The aqueous was extracted with CH₂Cl₂ (20 mL). This washing and extraction procedure was repeated three more times. The combined organic phase was washed with satd. NaHCO₃ (200 mL×2), brine, dried over anh. Na₂SO₄, filtered and concentrated to give a colorless oil. This crude product was purified by flash chromatography (0-10% MeOH in CH₂Cl₂) to afford allyl 2,2-dimethyl-4,11,16-trioxo-12-(2-(tritylamino)ethyl)-3,8,19,22-tetraoxa-5,12,15-triazapentacosan-25-oate as a thick colorless oil (11.275 g, 95% yield). LC/MS: MH⁺=789.7, Rt=1.05 min (2 min acidic method). ¹H NMR (400 MHz, DMSO) δ 7.98 (d, J=3.3 Hz, 1H), 7.82 (t, J=6.0 Hz, 1H), 7.40-7.33 (m, 6H), 7.28 (t, J=7.5 Hz, 6H), 7.22-7.14 (m, 3H), 6.71 (d, J=5.8 Hz, 1H), 5.90 (dddd, J=15.8, 9.4, 6.0, 4.6 Hz, 1H), 5.76 (s, 1H), 5.33-5.25 (m, 1H), 5.22-5.16 (m, 1H), 4.58-4.52 (m, 2H), 3.66-3.52 (m, 6H), 3.49 -3.35 (m, 6H), 3.27-2.96 (m, 6H), 2.82-2.53 (m, 5H), 2.36-2.20 (m, 3H), 2.15-2.01 (m, 2H), 1.37 (s, 9H).

### Synthesis of allyl 12-(2-aminoethyl)-2,2-dimethyl-4,11,16-trioxo-3,8,19,22-tetraoxa-5,12,15-triazapentacosan-25-oate

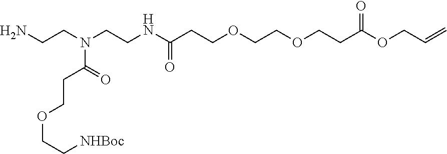 [SMILES: C=CCOC(=O)CCOCCOCCC(=O)NCCN(CCN)C(=O)CCOCCNC(=O)OC(C)(C)C]

To allyl 2,2-dimethyl-4,11,16-trioxo-12-(2-(tritylamino)ethyl)-3,8,19,22-tetraoxa-5,12,15-triazapentacosan-25-oate (8763 mg, 11.11 mmol) dissolved (via sonication) in trifluoroethanol (19 mL) was added acetic acid (19.07 mL, 333.2 mmol). The mixture was stirred at 60° C. for 2 h. The reaction mixture was cooled to rt and mixed with anh. toluene (100 mL) and concentrated. The azeotropical distillation with toluene was repeated two more times. To the residue was added toluene (8 mL) and the resulting solution was added dropwise to ice-cold 1:1 diethyl ether/heptane (400 mL) with rapid stirring. Stirring continued at 0° C. for 15 min. The clear top mother liquid was decanted and discarded. The bottom oily material was washed with fresh 1:1 ether/heptane (2×50 mL) and dried in vacuum to give an oil (7.718 g) as HOAc salt of the title compound. The material was dissolved with 1/9 TFE/CH₂Cl₂ (250 mL) and was washed with satd. NaHCO₃ (2×100 mL) until the aqueous was basic. The combined organic phase was washed with brine (75 mL). The aqueous was extracted with 1/9 TFE/CH₂Cl₂ (20 mL) twice. The combined organic phase was dried over anh. Na₂SO₄, filtered and concentrated to give a colorless oil as allyl 12-(2-aminoethyl)-2,2-dimethyl-4,11,16-trioxo-3,8,19,22-tetraoxa-5,12,15-triazapentacosan-25-oate (7.962 g, crude, 76.5% pure by wt. assuming 100% yield) used directly in the next step. LC/MS: MH⁺=547.5, Rt=0.73 min (2 min acidic method).

### Synthesis of 17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid

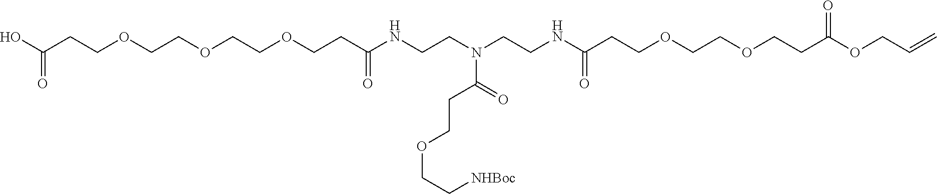 [SMILES: C=CCOC(=O)CCOCCOCCC(=O)NCCN(CCNC(=O)CCOCCOCCOCCC(=O)O)C(=O)CCOCCNC(=O)OC(C)(C)C]

To allyl 12-(2-aminoethyl)-2,2-dimethyl-4,11,16-trioxo-3,8,19,22-tetraoxa-5,12,15-triazapentacosan-25-oate prepared above (665 mg, 76.3% by Wt., 928 μmol) was added 3-(2-(2-(3-((2,5-dioxopyrrolidin-1-yl)oxy)-3-oxopropoxy)ethoxy)ethoxy)propanoic acid (387 mg, 1.11 mmol) dissolved in Acetonitrile (3.7 mL), and DIPEA (323 μL, 1.86 mmol). The mixture was stirred at rt/20 h, The volatile was removed via rotary evaporation. The resulting oil was diluted with DMSO (6 mL) and was purified by RP-HPLC (C18, ACN/water with 0.1% NH₄0H). The desired product-containing fractions (total ca. 112 mL) were diluted with EtOAc (75 mL) and acidified with 1 N HCl (3.5 mL). The organic phase was separated. The aqueous was extracted with EtOAc (2×30 mL). The combined EtOAc extract was mixed with heptane (20 mL) and the mixture was washed with brine (2×15 mL), dried over anh. Na₂SO₄, concentrated to provide a colorless oil as 17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid (274 mg, 37.8% yield). LC/MS: MH⁺=779.7, Rt=1.72 min (5 min acidic method). ¹H NMR (400 MHz, DMSO) δ 12.06 (s, 1H), 8.05-7.94 (m, 1H), 7.93-7.80 (m, 1H), 6.71 (d, J=6.0 Hz, 1H), 5.98-5.84 (m, 1H), 5.35-5.26 (m, 1H), 5.24-5.16 (m, 1H), 4.60-4.51 (m, 2H), 3.68 -3.53 (m, 10H), 3.53-3.41 (m, 12H), 3.40-3.24 (m, 8H, overlapping with DMSO), 3.17 (dt, J=16.1, 6.3 Hz, 4H), 3.09-3.00 (m, 2H), 2.61-2.53 (m, 4H), 2.44 (t, J=6.3 Hz, 2H), 2.33-2.24 (m, 4H), 1.37 (s, 9H). The aqueous layer was further extracted with 1/9 TFE/CH₂Cl₂ (3×15 mL) to provide additional title compound as a colorless oil (122 mg, 16.9% yield).

Synthesis of di-tert-butyl 13-(3-(2-((((9H-fluoren-9-yl)methoxy)carbonyl)amino)ethoxy)propanoyl)-4,7,10,16,19,22-hexaoxa-13-azapentacosanedioate

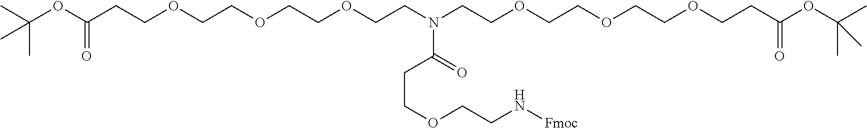

To a solution of 3-(2-((((9H-fluoren-9-yl)methoxy)carbonyl)amino)ethoxy)propanoic acid (CAS number: 1654740-73-4) (87 mg, 0.244 mmol) in DMF (0.50 mL) was added HATU (93 mg, 0.244 mmol) and DIPEA (0.205 mL, 1.172 mmol). The mixture was stirred at RT for 15 min and added into a solution of di-tert-butyl 4,7,10,16,19,22-hexaoxa-13-azapentacosanedioate (Broadpharm) (105 mg, 0.195 mmol) in DMF (0.2 mL). The mixture was stirred at RT for 1 hr and concentrated to remove the volatile. The residue was diluted with DMSO (2 ml) and was purified by RP-ISCO chromatography (ISCO Gold C18 Column, 100 g, Water/MeCN as eluent, 0.1% TFA as modifier) to afford di-tert-butyl 13-(3-(2-((((9H-fluoren-9-yl)methoxy)carbonyl)amino)ethoxy)propanoyl)-4,7,10,16,19,22-hexaoxa-13-azapentacosanedioate (135 mg, 79% yield) after lyophilization. LC/MS: MH⁺=875.8, Rt=1.33 min (2 min acidic method).

### Synthesis of 13-(3-(2-((((9H-fluoren-9-yl)methoxy)carbonyl)amino)ethoxy)propanoyl)-4,7,10,16,19,22-hexaoxa-13-azapentacosanedioic acid

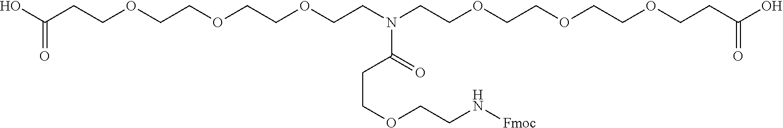

A mixture of di-tert-butyl 13-(3-(2-((((9H-fluoren-9-yl)methoxy)carbonyl)amino)ethoxy)propanoyl)-4,7,10,16,19,22-hexaoxa-13-azapentacosanedioate (135 mg, 0.154 mmol) and TFA (1.010 mL, 13.11 mmol) in DCM (2 mL) was stirred at RT for 2 hr. After adding DCE (3 ml), the mixture was concentrated on rotatory evaporator. The residue was diluted with DMSO (2 mL) and purified by RP-ISCO chromatography (100 G ISCO Gold C18 Column, Water/MeCN as eluent, 0.1% TFA as modifier) to afford 13-(3-(2-((((9H-fluoren-9-yl)methoxy)carbonyl)amino)ethoxy)propanoyl)-4,7,10,16,19,22-hexaoxa-13-azapentacosanedioic acid (120 mg, 99% yield) after lyophilization. LC/MS: MH⁺=763.5, Rt=0.97 min (2 min acidic method).

### Synthesis of tert-butyl (3-(bis(2-(2-(prop-2-yn-1-yloxy)ethoxy)ethyl)amino)-3-oxopropyl)carbamate

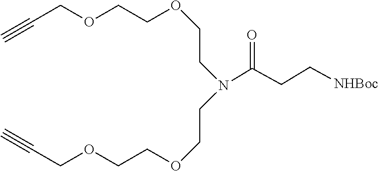 [SMILES: C#CCOCCOCCN(CCOCCOCC#C)C(=O)CCNC(=O)OC(C)(C)C]

To the solution of bis(2-(2-(prop-2-yn-1-yloxy)ethoxy)ethyl)amine (200 mg, 0.743 mmol), 3-((tert-butoxycarbonyl)amino)propanoic acid (148 mg, 0.780 mmol) in DMF (4 ml) was added HATU (296 mg, 0.780 mmol) and then DIPEA (389 μl, 2.228 mmol). The reaction mixture was stirred at RT for 1 h and was purified by a 150 g C18 RP column (Water/MeCN as eluent, 0.1% TFA as modifier) to afford after lyophilization tert-butyl (3-(bis(2-(2-(prop-2-yn-1-yloxy)ethoxy)ethyl)amino)-3-oxopropyl)carbamate (148 mg, 44% yield). LC/MS: MH⁺=441.3, Rt=0.92 min (2 min acidic method).

### Synthesis of 3-amino-N,N-bis(2-(2-(prop-2-yn-1-yloxy)ethoxy)ethyl)propanamide

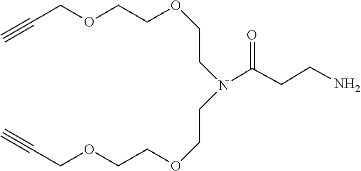 [SMILES: C#CCOCCOCCN(CCOCCOCC#C)C(=O)CCN]

TFA (0.568 mL, 7.38 mmol) was added to a solution of tert-butyl (3-(bis(2-(2-(prop-2-yn-1-yloxy)ethoxy)ethyl)amino)-3-oxopropyl)carbamate (65 mg, 0.15 mmol) in dichloromethane (0.6 mL). The mixture was stirred at RT for 1 hr. After adding DCE (3 ml), the mixture was concentrated on rotatory evaporator. 2-3 ml ether was added to the residue and sonicated, and the ether was removed to afford 3-amino-N,N-bis(2-(2-(prop-2-yn-1-yloxy)ethoxy)ethyl)propanamide (66.4 mg, 99% yield). LC/MS: MH⁺=421.2, Rt=0.52 min (2 min acidic method).

### Synthesis of 4-(4-((2S,5S)-23-(3-(2-((tert-butoxycarbonyl)amino)ethoxy)propanoyl)-5-isopropyl-4,7,19,27,36-pentaoxo-2-(3-ureidopropyl)-10,13,16,30,33,37-hexaoxa-3,6,20,23,26-pentaazatetracont-39-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

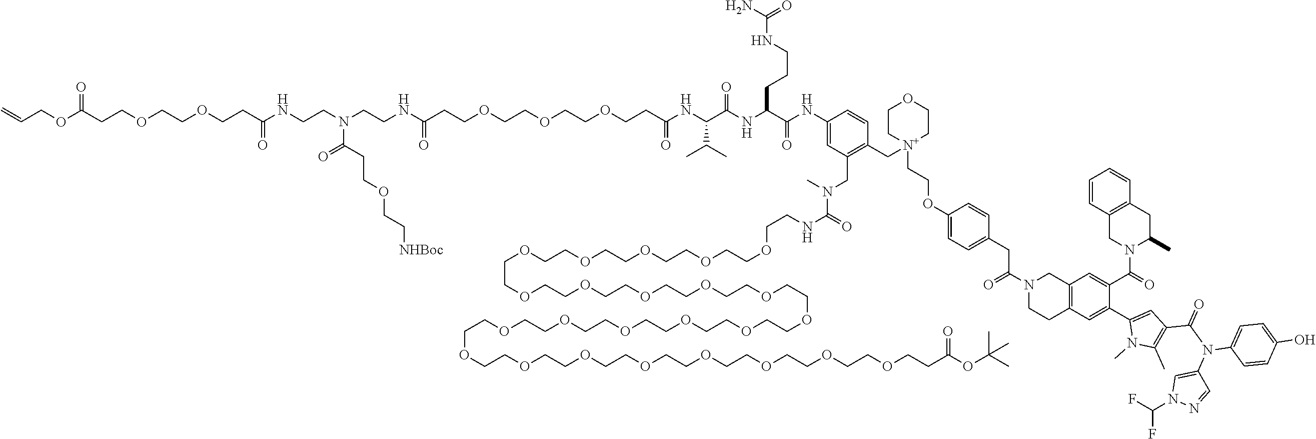 [SMILES: C=CCOC(=O)CCOCCOCCC(=O)NCCN(CCNC(=O)CCOCCOCCOCCC(=O)N[C@H](C(=O)N[C@@H](CCCNC(N)=O)C(=O)Nc1ccc(C[N+]2(CCOc3ccc(CC(=O)N4CCc5cc(-c6cc(C(=O)N(c7ccc(O)cc7)c7cnn(C(F)F)c7)c(C)n6C)c(C(=O)N6Cc7ccccc7C[C@H]6C)cc5C4)cc3)CCOCC2)c(CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)c1)C(C)C)C(=O)CCOCCNC(=O)OC(C)(C)C]

GENERAL PROCEDURE #3: a solution of 4-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (230 mg, 90.8 μmol), HOAT (14.2 mg, 104.5 μmol), 17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid (81.4 mg, 104.5 μmol, 1.15 equiv.) and DIEA (79.1 μL, 454 μmol, 5.0 equiv.) in DMF (1 mL) was stirred for 5 minutes at which time PyBOP (54.4 mg, 104.5 μmol, 1.15 equiv.) was added. After stirring an additional 30 minutes, the solution was diluted with DMSO and purified by ISCO RP-HPLC. Upon lyophilization, 4-(4-((2S,5S)-23-(3-(2-((tert-butoxycarbonyl)amino)ethoxy)propanoyl)-5-isopropyl-4,7,19,27,36-pentaoxo-2-(3-ureidopropyl)-10,13,16,30,33,37-hexaoxa-3,6,20,23,26-pentaazatetracont-39-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (294 mg, 95%) was obtained. HRMS: M⁺=3290.8000, Rt=2.52 min (5 min acidic method).

### Synthesis of 4-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

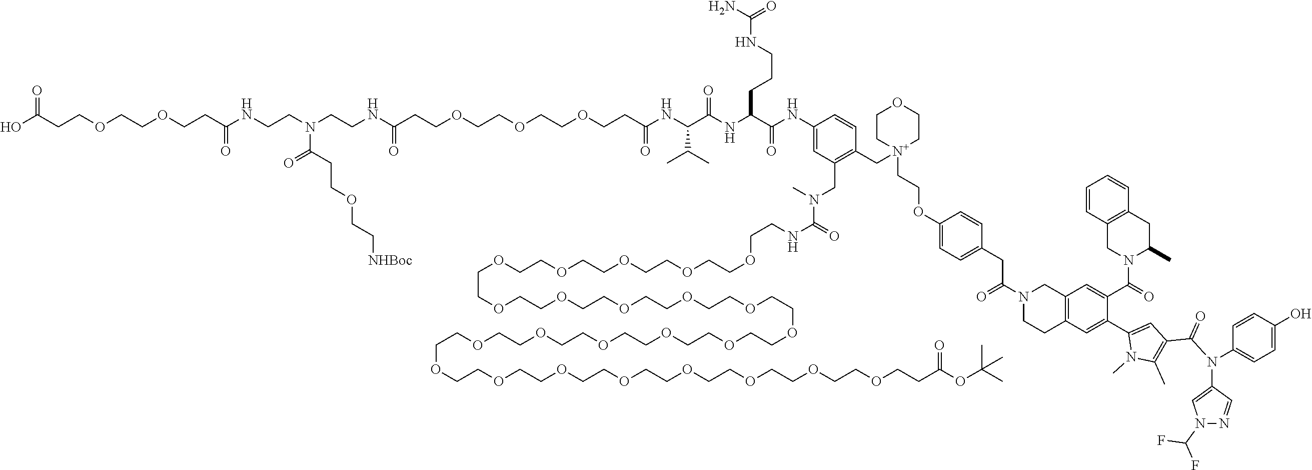 [SMILES: Cc1c(C(=O)N(c2ccc(O)cc2)c2cnn(C(F)F)c2)cc(-c2cc3c(cc2C(=O)N2Cc4ccccc4C[C@H]2C)CN(C(=O)Cc2ccc(OCC[N+]4(Cc5ccc(NC(=O)[C@H](CCCNC(N)=O)NC(=O)[C@@H](NC(=O)CCOCCOCCOCCC(=O)NCCN(CCNC(=O)CCOCCOCCC(=O)O)C(=O)CCOCCNC(=O)OC(C)(C)C)C(C)C)cc5CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)CCOCC4)cc2)CC3)n1C]

Following GENERAL PROCEDURE #2 using 4-(4-((2S,5S)-23-(3-(2-((tert-butoxycarbonyl)amino)ethoxy)propanoyl)-5-isopropyl-4,7,19,27,36-pentaoxo-2-(3-ureidopropyl)-10,13,16,30,33,37-hexaoxa-3,6,20,23,26-pentaazatetracont-39-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (274 mg, 80.4 μmol), pyrollidine (46 p L, 563 μmol) and tetrakis(triphenylphosphine)palladium (10 mg, 8.6 μmol), 4-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (180 mg, 62% yield) was obtained. HRMS: M⁺=3250.7600, Rₜ=2.43 min (5 min acidic method).

### Synthesis of 4-(4-((6S,9S,42S,45S)-1-amino-24-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

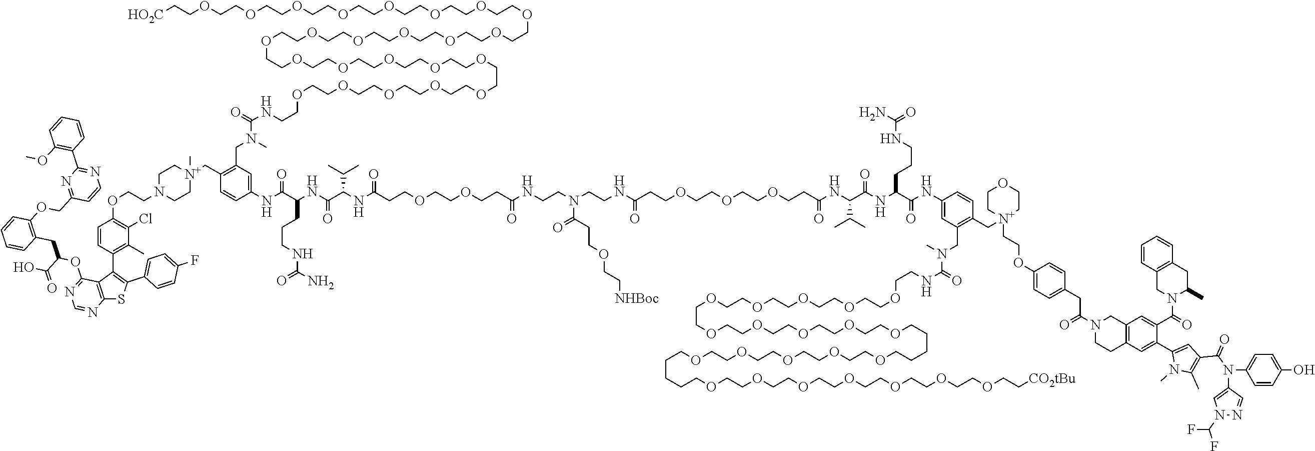

GENERAL PROCEDURE #4: 4-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (170 mg, 50.5 μmol), TSTU (16 mg, 53 μmol) and DIEA (132 p L, 757 μmol) in DMF (1.5 mL) were stirred for 15 minutes at which time 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium TFA salt (148.9 mg, 55.5 μmol) was added. After stirring for an additional 4 hours, the solution was diluted with DMSO and purified by ISCO RP-HPLC. Upon lyophilization, 4-(4-((6S,9S,42S,45S)-1-amino-24-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (188 mg, 61% yield) was obtained. HRMS: (M⁺²- H⁺)+=5682.9502, Rt=2.68 min (5 min acidic method).

### Synthesis of 4-(4-((6S,9S,42S,45S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-24-(1-(2,5-dioxo-2,5-dihydro-1H-pyrrol-1-yl)-15-oxo-3,6,9,12,19-pentaoxa-16-azadocosan-22-oyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate P1-L17-P3

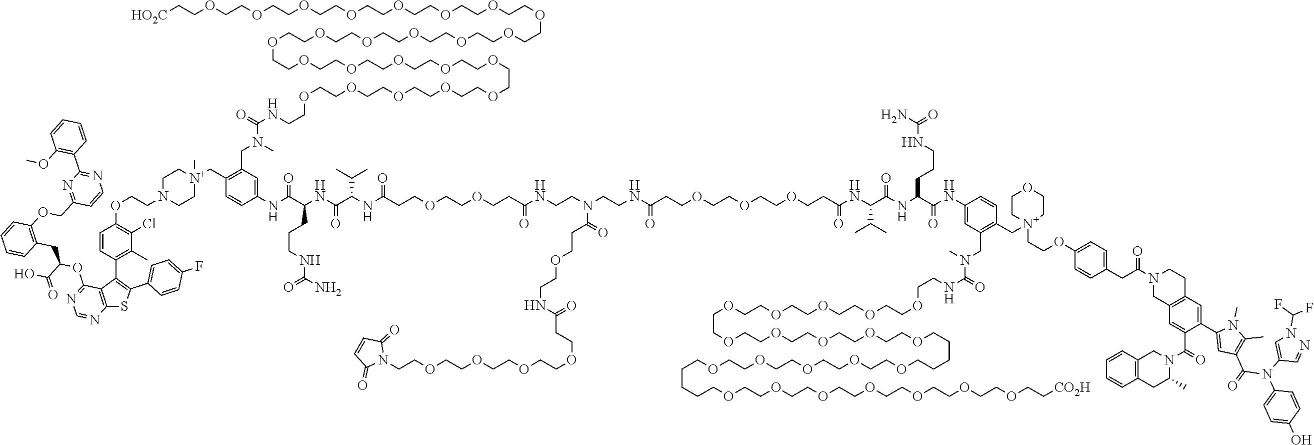

GENERAL PROCEDURE #5: 4-(4-((6S,9S,42S,45S)-1-amino-24-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (185 mg, 31.3 μmol) was treated with 4 mL of 25% TFA/CH₂Cl₂ with 0.1% Triethylsilane for 1 hour at which time the volatiles were removed in vacuo. The residue was triturated with diethyl ether. The residue was dissolved in DMF (1.5 mL) and 2,5-dioxopyrrolidin-1-yl 1-(2,5-dioxo-2,5-dihydro-1H-pyrrol-l-yl)-3,6,9,12-tetraoxapentadecan-15-oate (28.5 mg, 64.4 μmol) and DIEA (84 μL, 483 μmol) were added. After stirring for 1 hour the solution was diluted with DMSO and purified by ISCO RP-HPLC. Upon lyophilization, 4-(4-((6S,9S,42S,45S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-24-(1-(2,5-dioxo-2,5-dihydro-1H-pyrrol-l-yl)-15-oxo-3,6,9,12,19-pentaoxa-16-azadocosan-22-oyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (110 mg, 57% yield) was obtained. HRMS: (M⁺²- H⁺)+=5853.9302, Rt=2.47 min (5 min acidic method).

### Synthesis of 1-(4-((29S,32S)-1-amino-29-isopropyl-27,30-dioxo-32-(3-ureidopropyl)-3,6,9,12,15,18,21,24-octaoxa-28,31-diazatritriacontan-33-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

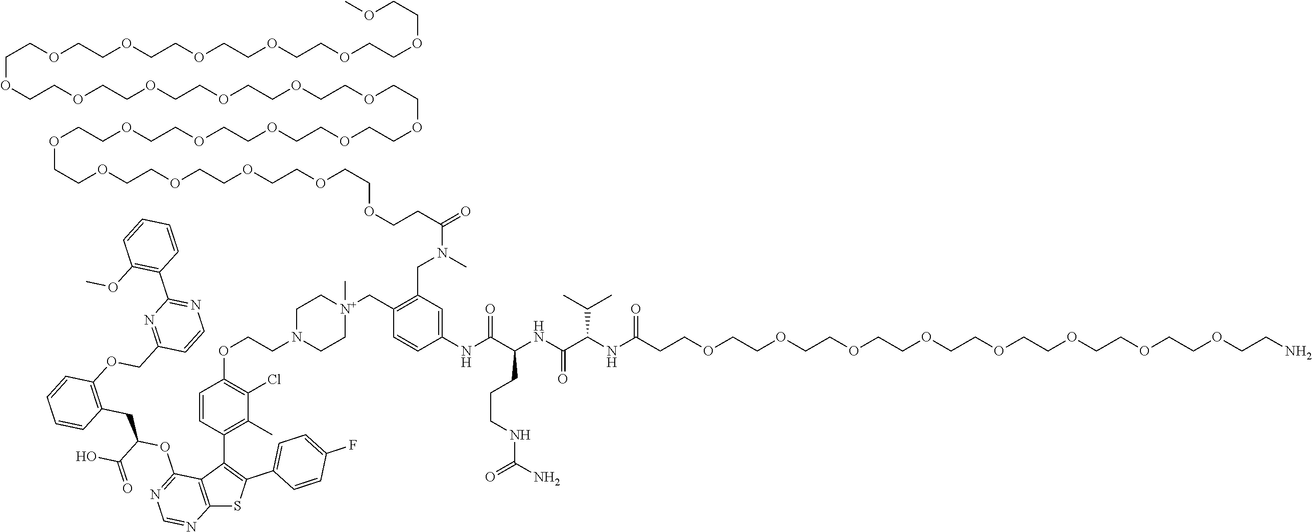 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCOCCOCCOCCOCCOCCOCCOCCN)C(C)C)ccc1C[N+]1(C)CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1]

GENERAL PROCEDURE #6: to a solution of 1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium (77 mg, 28 μmol), 1-(9H-fluoren-9-yl)-3-oxo-2,7,10,13,16,19,22,25,28-nonaoxa-4-azahentriacontan-31-oic acid (28.2 mg, 42 μmol) and HATU (11.8 mg, 31 μmol) in DMF (1 mL) was added DIEA (30 μL, 170 μmol). After stirring for 30 min, 2.0 M Dimethylamine in MeOH (141 μL, 282 μmol) was added and after an additional 1 hour stirring the solution was diluted with DMSO and purified by ISCO RP-HPLC. Upon lyophilization, 1-(4-((29S,32S)-1-amino-29-isopropyl-27,30-dioxo-32-(3-ureidopropyl)-3,6,9,12,15,18,21,24-octaoxa-28,31-diazatritriacontan-33-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (66 mg, 74% yield) was obtained. HRMS: M⁺=2801.4199, Rt=2.18 min (5 min acidic method).

### Synthesis of 1-(4-((10S,41S,44S)-10-amino-41-isopropyl-2,2-dimethyl-4,11,39,42-tetraoxo-44-(3-ureidopropyl)-3,15,18,21,24,27,30,33,36-nonaoxa-5,12,40,43-tetraazapentatetracontan-45-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

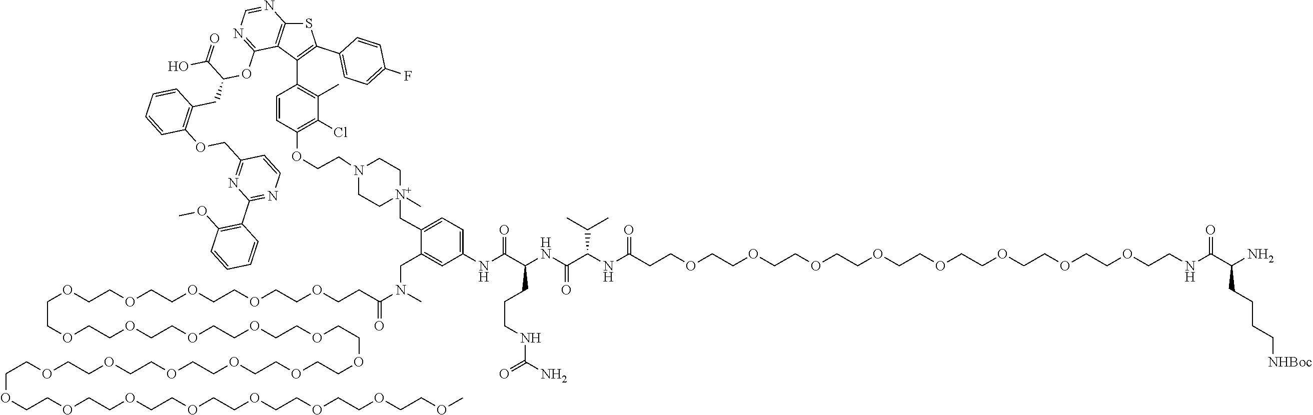 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCOCCOCCOCCOCCOCCOCCOCCNC(=O)[C@@H](N)CCCCNC(=O)OC(C)(C)C)C(C)C)ccc1C[N+]1(C)CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1]

Following GENERAL PROCEDURE #6 using 1-(4-((29S,32S)-1-amino-29-isopropyl-27,30-dioxo-32-(3-ureidopropyl)-3,6,9,12,15,18,21,24-octaoxa-28,31-diazatritriacontan-33-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (30 mg, 9.9 μmol), N2-(((9H-fluoren-9-yl)methoxy)carbonyl)-N6-(tert-butoxycarbonyl)-L-lysine (6 mg, 13 μmol) and HATU (4.1 mg, 10.9 μmol) and then 2.0 M Dimethylamine in MeOH (99 μL, 198 μmol),1-(4-((10S,41S,44S)-10-amino-41-isopropyl-2,2-dimethyl-4,11,39,42-tetraoxo-44-(3-ureidopropyl)-3,15,18,21,24,27,30,33,36-nonaoxa-5,12,40,43-tetraazapentatetracontan-45-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (22.3 mg, 67% yield) was obtained. HRMS: M⁺=3029.5701, Rt=2.25 min (5 min acidic method).

### Synthesis of 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((2S,5S)-37-((2,5-dioxopyrrolidin-1-yl)oxy)-5-isopropyl-4,7,37-trioxo-2-(3-ureidopropyl)-10,13,16,19,22,25,28,31,34-nonaoxa-3,6-diazaheptatriacontanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate

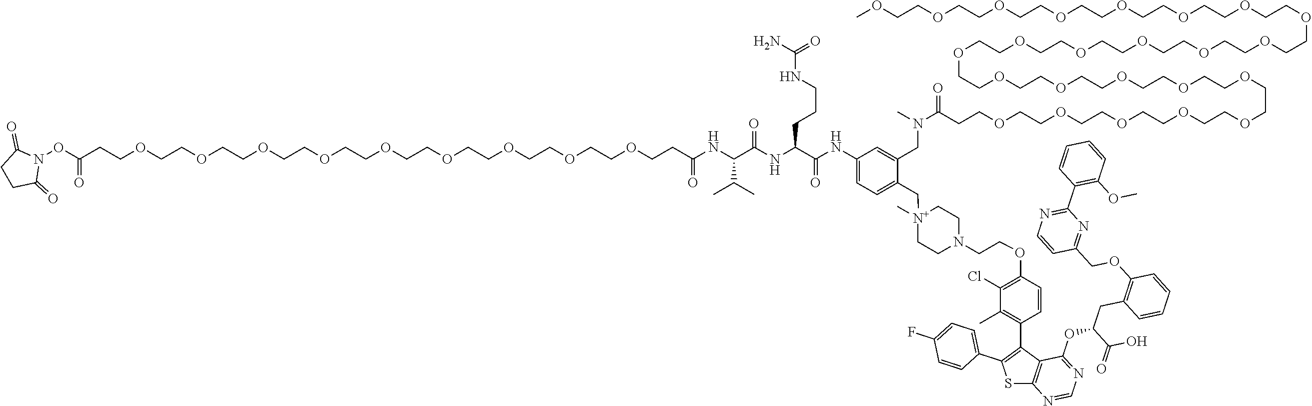 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCOCCOCCOCCOCCOCCOCCOCCOCCC(=O)ON2C(=O)CCC2=O)C(C)C)ccc1C[N+]1(C)CCN(CCOc2ccc(-c3c(-c4ccc(F)cc4)sc4ncnc(O[C@H](Cc5ccccc5OCc5ccnc(-c6ccccc6OC)n5)C(=O)O)c34)c(C)c2Cl)CC1]

To a solution of 1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (36.5 mg, 12 μmol) and bis(2,5-dioxopyrrolidin-1-yl) 4,7,10,13,16,19,22,25,28-nonaoxahentriacontanedioate (29.9 mg, 42 μmol) in DMF (0.5 mL) was added DIEA (13 μL, 72 μmol). After stirring for 1 hour, the solution was diluted with DMSO and purified by ISCO RP-HPLC. Upon lyophilization, 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((2S,5S)-37-((2,5-dioxopyrrolidin-1-yl)oxy)-5-isopropyl-4,7,37-trioxo-2-(3-ureidopropyl)-10,13,16,19,22,25,28,31,34-nonaoxa-3,6-diazaheptatriacontanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (34.5 mg, 93% yield) was obtained. HRMS: M⁺=2971.4399, Rt=2.39 min (5 min acidic method).

### Synthesis of 1,1′-(((((2S,5S,36S,70S,73S)-36-(4-((tert-butoxycarbonyl)amino)butyl)-5,70-diisopropyl-4,7,35,38,68,71-hexaoxo-2,73-bis(3-ureidopropyl)-10,13,16,19,22,25,28,31,41,44,47,50,53,56,59,62,65-heptadecaoxa-3,6,34,37,69,72-hexaazatetraheptacontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

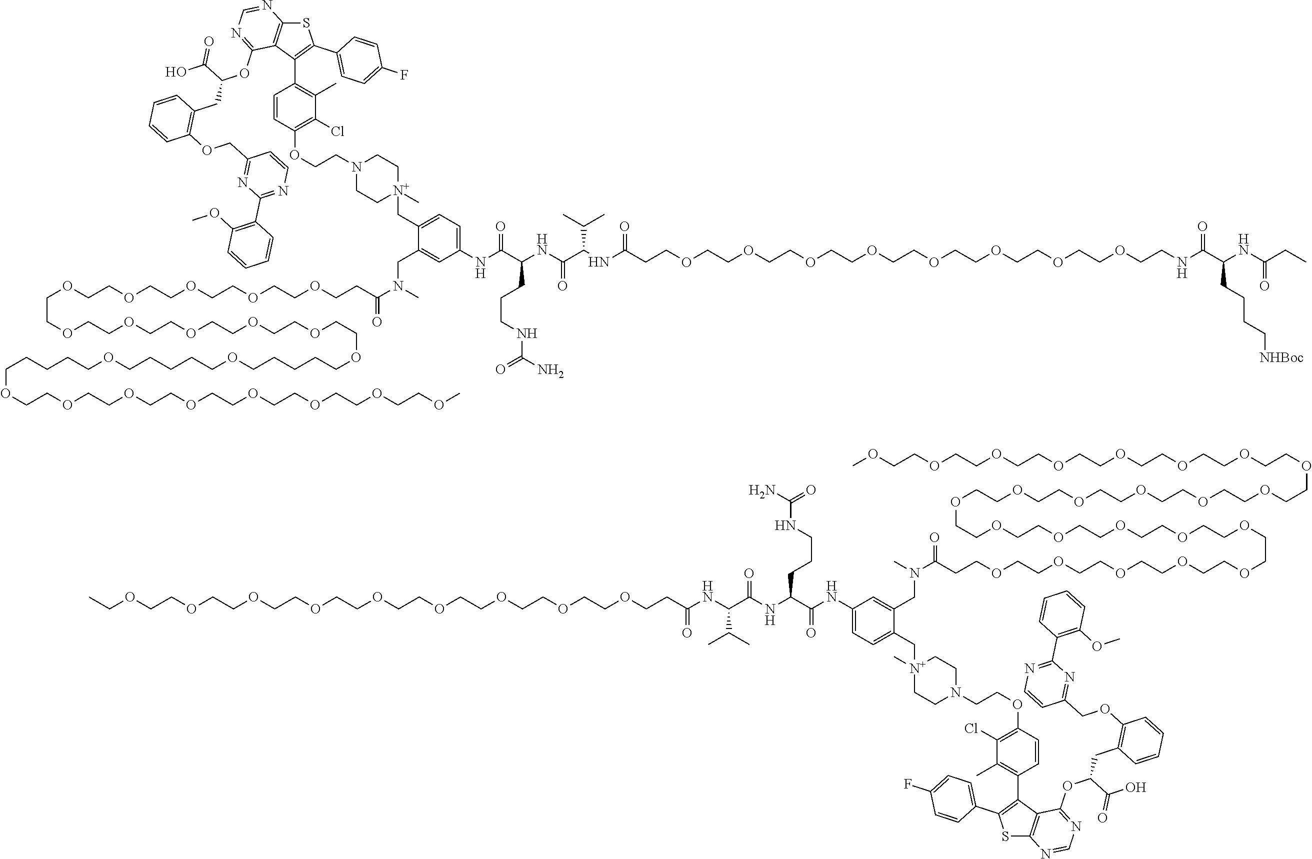

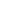

To a solution of 1-(4-((10S,41S,44S)-10-amino-41-isopropyl-2,2-dimethyl-4,11,39,42-tetraoxo-44-(3-ureidopropyl)-3,15,18,21,24,27,30,33,36-nonaoxa-5,12,40,43-tetraazapentatetracontan-45-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (22.3 mg, 6.6 μmol) and 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((2S,5S)-37-((2,5-dioxopyrrolidin-1-yl)oxy)-5-isopropyl-4,7,37-trioxo-2-(3-ureidopropyl)-10,13,16,19,22,25,28,31,34-nonaoxa-3,6-diazaheptatriacontanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (22.3 mg, 6.6 μmol) in DMF (1 mL) was added DIEA (12 μL, 66 μmol). After stirring for 5 hours, the solution was diluted with DMSO and purified by ISCO RP-HPLC. Upon lyophilization, 1,1′-(((((2S,5S,36S,70S,73S)-36-(4-((tert-butoxycarbonyl)amino)butyl)-5,70-diisopropyl-4,7,35,38,68,71-hexaoxo-2,73-bis(3-ureidopropyl)-10,13,16,19,22,25,28,31,41,44,47,50,53,56,59,62,65-heptadecaoxa-3,6,34,37,69,72-hexaazatetraheptacontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (24.7 mg, 61% yield) was obtained. HRMS: (M⁺²- H⁺)+=5884.9502, Rt=2.70 min (5 min acidic method).

### Synthesis of 1,1′-(((((2S,5S,36S,70S,73S)-36-(1-(2,5-dioxo-2,5-dihydro-1H-pyrrol-1-yl)-27-oxo-3,6,9,12,15,18,21,24-octaoxa-28-azadotriacontan-32-yl)-5,70-diisopropyl-4,7,35,38,68,71-hexaoxo-2,73-bis(3-ureidopropyl)-10,13,16,19,22,25,28,31,41,44,47,50,53,56,59,62,65-heptadecaoxa-3,6,34,37,69,72-hexaazatetraheptacontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L8-P1

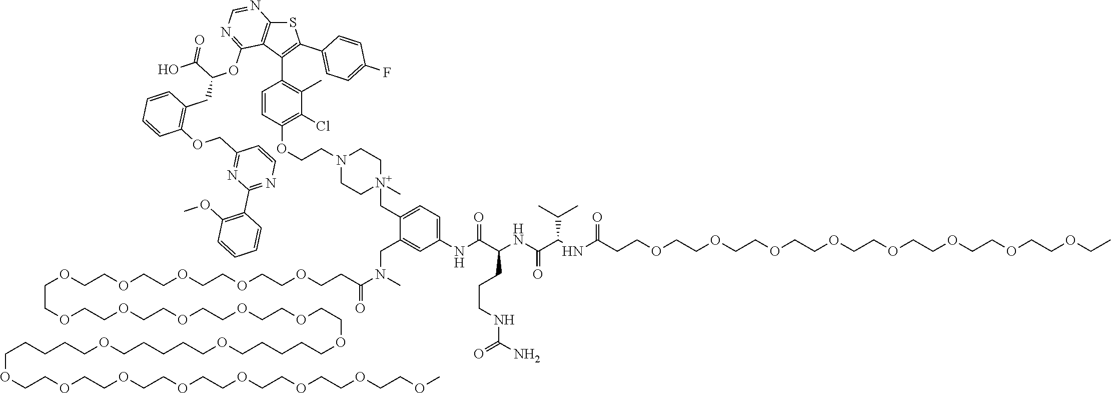

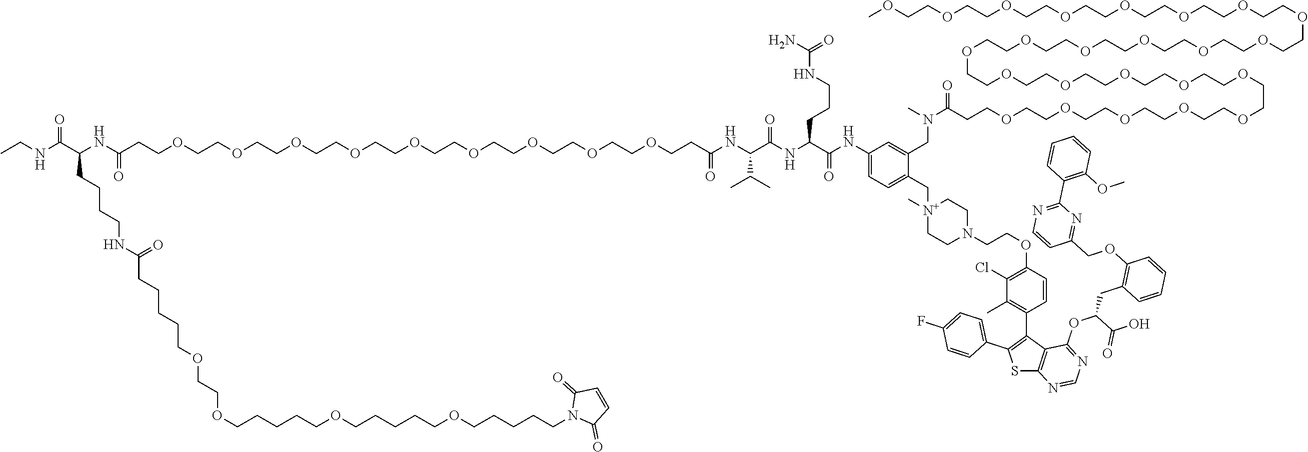 [SMILES: CCNC(=O)[C@H](CCCCNC(=O)CCCCCOCCOCCCCCOCCCCCOCCCCCN1C(=O)C=CC1=O)NC(=O)CCOCCOCCOCCOCCOCCOCCOCCOCCOCCC(=O)N[C@H](C(=O)N[C@@H](CCCNC(N)=O)C(=O)Nc1ccc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)c(CN(C)C(=O)CCOCCOCCOCCOCCOCCOCCOCCOCCOCCOCCOCCOCCOCCOCCOCCOCCOCCOCCOCCOCCOCCOCCOCCOC)c1)C(C)C]

Following the GENERAL PROCEDURE #5 using 1,1′-(((((2S,5S,36S,70S,73S)-36-(4-((tert-butoxycarbonyl)amino)butyl)-5,70-diisopropyl-4,7,35,38,68,71-hexaoxo-2,73-bis(3-ureidopropyl)-10,13,16,19,22,25,28,31,41,44,47,50,53,56,59,62,65-heptadecaoxa-3,6,34,37,69,72-hexaazatetraheptacontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (24.7 mg, 4 μmol), 2,5-dioxopyrrolidin-1-yl 1-(2,5-dioxo-2,5-dihydro-1 H-pyrrol-1-yl)-3,6,9,12,15,18,21,24-octaoxaheptacosan-27-oate (5 mg, 8 μmol) and DIEA (14.1 p L, 81 μmol), 1,1′-(((((2S,5S,36S,70S,73S)-36-(1-(2,5-dioxo-2,5-dihydro-1 H-pyrrol-1 -yl)-27-oxo-3,6,9,12,15,18,21,24-octaoxa-28-azadotriacontan-32-yl)-5,70-diisopropyl-4,7,35,38,68,71-hexaoxo-2,73-bis(3-ureidopropyl)-10,13,16,19,22,25,28,31,41,44,47,50,53,56,59,62,65-heptadecaoxa-3,6,34,37,69,72-hexaazatetraheptacontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (8.4 mg, 30% yield) was obtained. HRMS: (M⁺²- H⁺)+=6288.0200, Rt=2.59 min (5 min acidic method).

### Synthesis of 1,1′-(((((2S,5S,27S,32S,35S)-27-amino-5,32-diisopropyl-4,7,26,30,33-pentaoxo-2,35-bis(3-ureidopropyl)-10,13,16,19,22-pentaoxa-3,6,25,31,34-pentaazahexatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

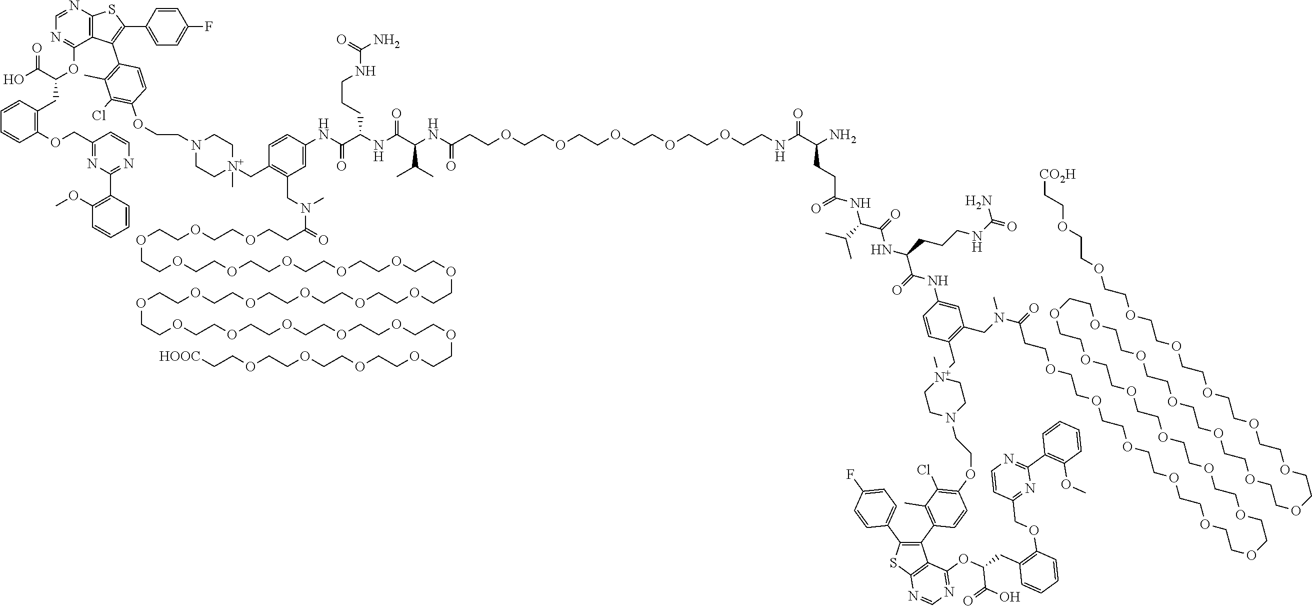

Following the GENERAL PROCEDURE #6 using 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (98 mg, 39 μmol), (S)-21-((((9H-fluoren-9-yl)methoxy)carbonyl)amino)-20-oxo-4,7,10,13,16-pentaoxa-19-azatetracosanedioic acid (10 mg, 15 μmol), HATU (11.5 mg, 30 μmol), DIEA (26.4 p L, 151 μmol) and then 2.0 M Dimethylamine in MeOH (480 μL, 960 μmol), 1,1′-(((((2S,5S,27S,32S,35S)-27-amino-5,32-diisopropyl-4,7,26,30,33-pentaoxo-2,35-bis(3-ureidopropyl)-10,13,16,19,22-pentaoxa-3,6,25,31,34-pentaazahexatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (26.5 mg, 32% yield) was obtained. HRMS: (M⁺²- H⁺)+=5361.5601, Rt=2.50 min (5 min acidic method).

### Synthesis of 1,1′-(((((2S,5S,27S,32S,35S)-27-(1-(2,5-dioxo-2,5-dihydro-1H-pyrrol-1-yl)-3,6,9,12,15,18,21,24-octaoxaheptacosan-27-amido)-5,32-diisopropyl-4,7,26,30,33-pentaoxo-2,35-bis(3-ureidopropyl)-10,13,16,19,22-pentaoxa-3,6,25,31,34-pentaazahexatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L1-P1

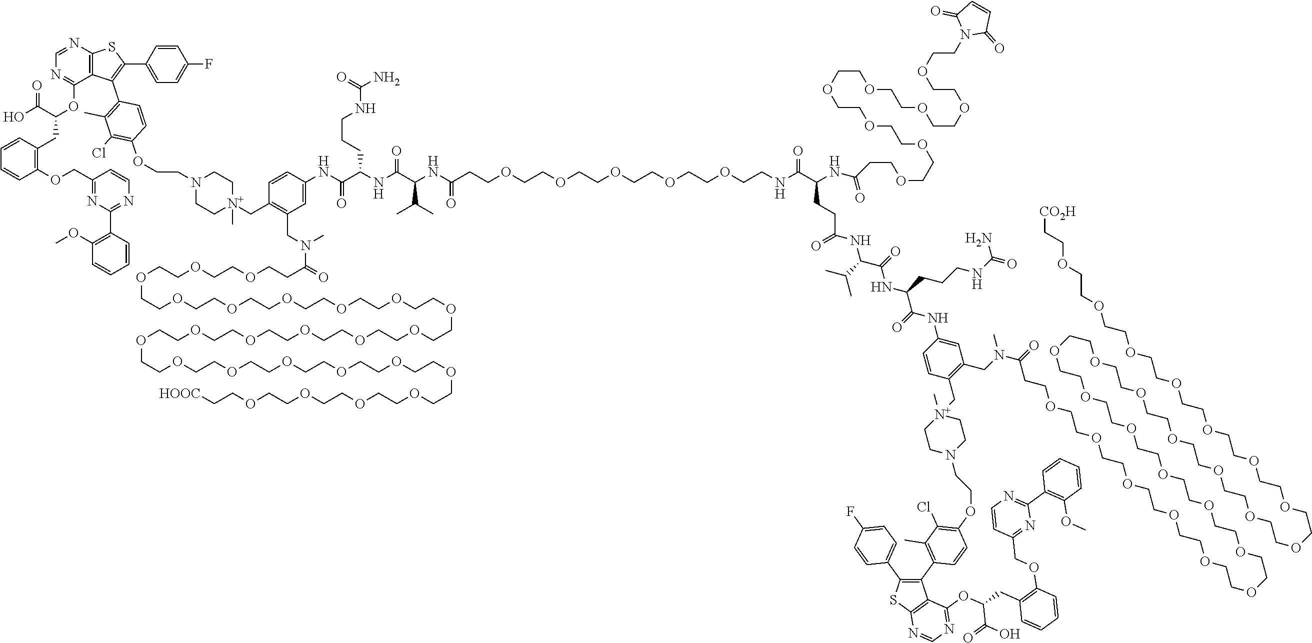

Following the second half of GENERAL PROCEDURE #5 using 1,1′-(((((2S,5S,27S,32S,35S)-27-amino-5,32-diisopropyl-4,7,26,30,33-pentaoxo-2,35-bis(3-ureidopropyl)-10,13,16,19,22-pentaoxa-3,6,25,31,34-pentaazahexatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (26.5 mg, 4.7 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (14.7 mg, 24 μmol) and DIEA (16.5 mg, 95 μmol), 1,1′-(((((2S,5S,27S,32S,35S)-27-(1-(2,5-dioxo-2,5-dihydro-1H-pyrrol-l-yl)-3,6,9,12,15,18,21,24-octaoxaheptacosan-27-amido)-5,32-diisopropyl-4,7,26,30,33-pentaoxo-2,35-bis(3-ureidopropyl)-10,13,16,19,22-pentaoxa-3,6,25,31,34-pentaazahexatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (10 mg, 34% yield) was obtained. HRMS: (M⁺²- H⁺)+=5864.7998, Rt=2.65 min (5 min acidic method).

### Synthesis of 1,1′-(((((2S,5S,30S,35S,38S)-30-amino-5,35-diisopropyl-4,7,29,33,36-pentaoxo-2,38-bis(3-ureidopropyl)-10,13,16,19,22,25-hexaoxa-3,6,28,34,37-pentaazanonatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

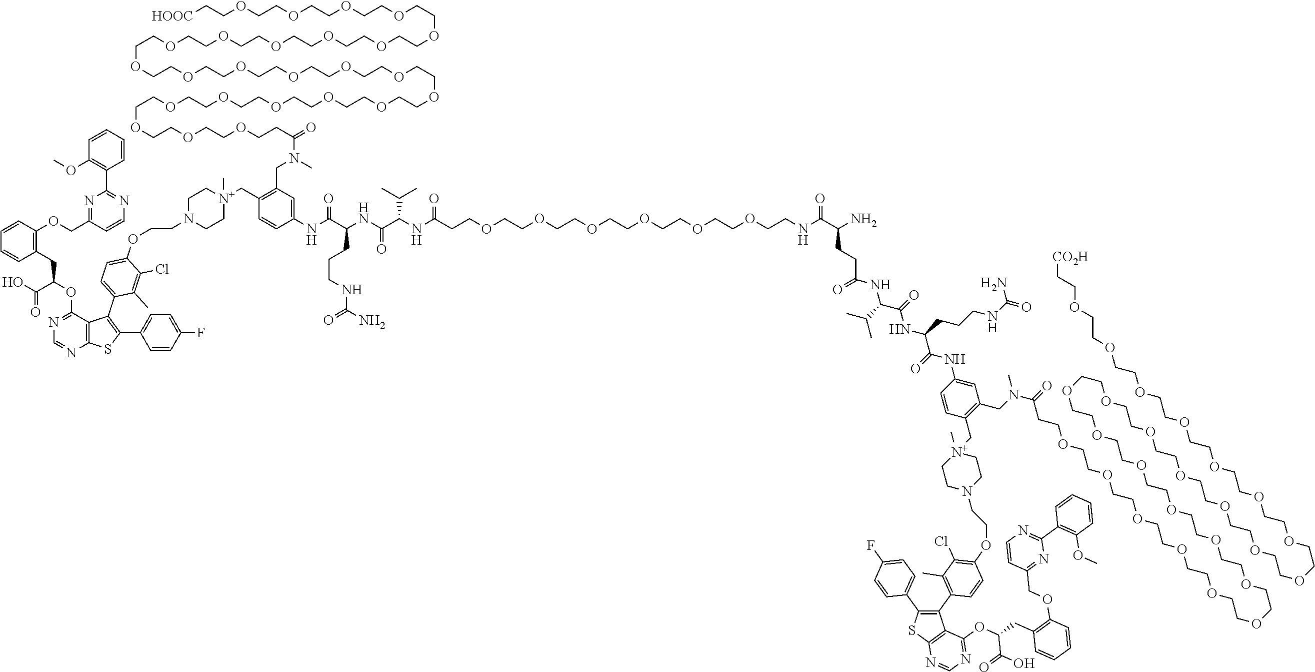

Following the GENERAL PROCEDURE #6 using 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (146 mg, 59 μmol), (S)-24-((((9H-fluoren-9-yl)methoxy)carbonyl)amino)-23-oxo-4,7,10,13,16,19-hexaoxa-22-azaheptacosanedioic acid (16 mg, 23 μmol), HATU (17.3 mg, 45 μmol), DIEA (39.7 p L, 227 μmol) and then 2.0 M Dimethylamine in MeOH (862 μL, 1725 μmol), 1,1′-(((((2S,5S,30S,35S,38S)-30-amino-5,35-diisopropyl-4,7,29,33,36-pentaoxo-2,38-bis(3-ureidopropyl)-10,13,16,19,22,25-hexaoxa-3,6,28,34,37-pentaazanonatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (66 mg, 43% yield) was obtained. HRMS: (M⁺²- H⁺)+=5405.6001, Rt=2.50 min (5 min acidic method).

### Synthesis of 1,1′-(((((2S,5S,30S,35S,38S)-30-(1-(2,5-dioxo-2,5-dihydro-1H-pyrrol-1-yl)-3,6,9,12,15,18,21,24-octaoxaheptacosan-27-amido)-5,35-diisopropyl-4,7,29,33,36-pentaoxo-2,38-bis(3-ureidopropyl)-10,13,16,19,22,25-hexaoxa-3,6,28,34,37-pentaazanonatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L2-P1

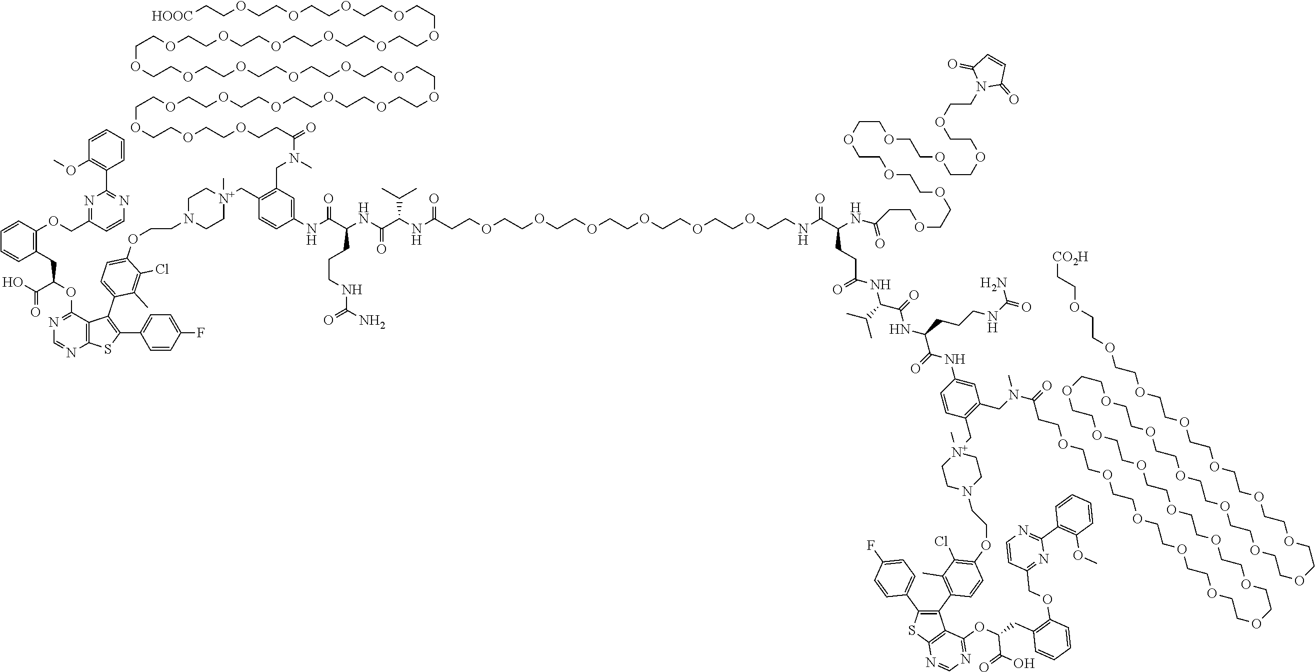

Following the second half of GENERAL PROCEDURE #5 using 1,1′-(((((2S,5S,30S,35S,38S)-30-amino-5,35-diisopropyl-4,7,29,33,36-pentaoxo-2,38-bis(3-ureidopropyl)-10,13,16,19,22,25-hexaoxa-3,6,28,34,37-pentaazanonatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (49.4 mg, 8.6 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (26.6 mg, 43 μmol) and DIEA (30 p L, 172 μmol), 1,1′-(((((2S,5S,30S,35S,38S)-30-(1-(2,5-dioxo-2,5-dihydro-1H-pyrrol-l-yl)-3,6,9,12,15,18,21,24-octaoxaheptacosan-27-amido)-5,35-diisopropyl-4,7,29,33,36-pentaoxo-2,38-bis(3-ureidopropyl)-10,13,16,19,22,25-hexaoxa-3,6,28,34,37-pentaazanonatriacontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (22.5 mg, 42% yield) was obtained. HRMS: (M⁺²- H⁺)+=5908.8501, Rt=2.64 min (5 min acidic method).

### Synthesis of 1-(4-((S)-2-((S)-2-amino-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

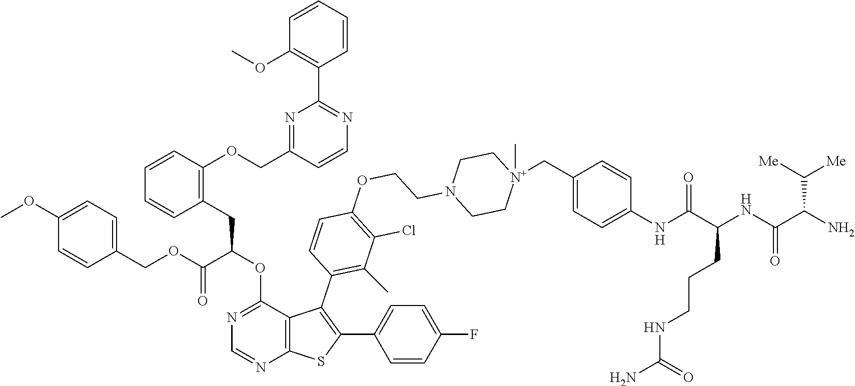 [SMILES: COc1ccc(COC(=O)[C@@H](Cc2ccccc2OCc2ccnc(-c3ccccc3OC)n2)Oc2ncnc3sc(-c4ccc(F)cc4)c(-c4ccc(OCCN5CC[N+](C)(Cc6ccc(NC(=O)[C@H](CCCNC(N)=O)NC(=O)[C@@H](N)C(C)C)cc6)CC5)c(Cl)c4C)c23)cc1]

To a solution of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (380 mg, 382 μmol), tetrabutylammonium iodide (169 mg, 458 μmol) and (9H-fluoren-9-yl)methyl ((S)-1-(((S)-1-((4-(chloromethyl)phenyl)amino)-1-oxo-5-ureidopentan-2-yl)amino)-3-methyl-1-oxobutan-2-yl)carbamate (284 mg, 458 μmol) in DMF (3 mL) was added DIEA (133 μL, 763 mmol). After stirring for 16 hours, 2.0 M Dimethyl amine in MeOH (5.7 mL, 11.4 mmol) was added. After stirring for 1 hour, the solution was diluted with DMSO and purified by ISCO RP-HPLC. Upon lyophilization, 1-(4-((S)-2-((S)-2-amino-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate was obtained. LCMS: (M⁺²/2)+=679.2, Rt=2.19 min (5 min acidic method).

### Synthesis of 1-(4-((6S,9S,12S)-6-(aminomethyl)-9-isopropyl-2,2-dimethyl-4,7,10-trioxo-12-(3-ureidopropyl)-3-oxa-5,8,11-triazatridecan-13-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

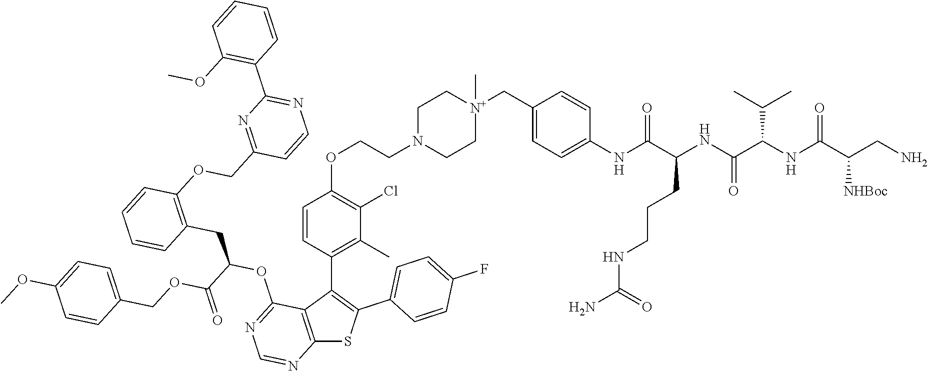 [SMILES: COc1ccc(COC(=O)[C@@H](Cc2ccccc2OCc2ccnc(-c3ccccc3OC)n2)Oc2ncnc3sc(-c4ccc(F)cc4)c(-c4ccc(OCCN5CC[N+](C)(Cc6ccc(NC(=O)[C@H](CCCNC(N)=O)NC(=O)[C@@H](NC(=O)[C@H](CN)NC(=O)OC(C)(C)C)C(C)C)cc6)CC5)c(Cl)c4C)c23)cc1]

Following the GENERAL PROCEDURE #6 using 1-(4-((S)-2-((S)-2-amino-3-methylbutanamido)-5-ureidopentanamido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (82 mg, 48 μmol), (S)-3-((((9H-fluoren-9-yl)methoxy)carbonyl)amino)-2-((tert-butoxycarbonyl)amino)propanoic acid (24.7 mg, 58 μmol), HATU (20.2 mg, 53 μmol), DIEA (51 μL, 289 μmol) and then 2.0 M Dimethylamine in MeOH (723 μL, 1447 μmol), 1-(4-((6S,9S,12S)-6-(aminomethyl)-9-isopropyl-2,2-dimethyl-4,7,10-trioxo-12-(3-ureidopropyl)-3-oxa-5,8,11-triazatridecan-13-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (23.7 mg, 26% yield) was obtained. HRMS: M⁺=1542.6500, Rt=2.42 min (5 min acidic method).

### Synthesis of 1,1′-(((((2S,5S,8S,32S,35S,38S)-20-(3-(2-((((9H-fluoren-9-yl)methoxy)carbonyl)amino)ethoxy)propanoyl)-8,32-diamino-5,35-diisopropyl-4,7,11,18,22,29,33,36-octaoxo-2,38-bis(3-ureidopropyl)-14,26-dioxa-3,6,10,17,20,23,30,34,37-nonaazanonatriacontanedioyl)bis(azanediyl))bis(4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1

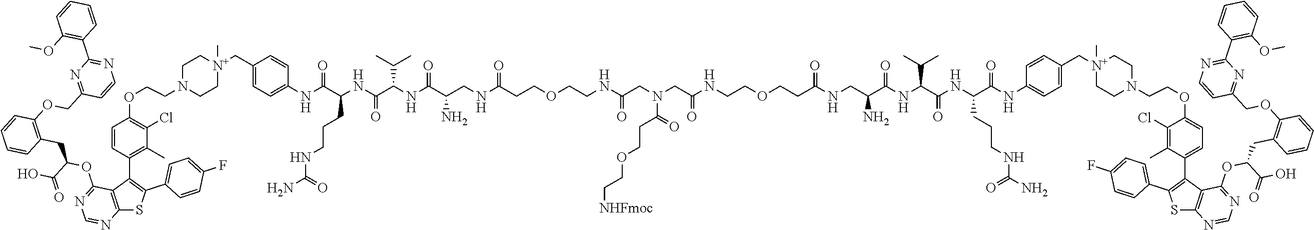

To a solution of 10-(3-(2-((((9H-fluoren-9-yl)methoxy)carbonyl)amino)ethoxy)propanoyl)-8,12-dioxo-4,16-dioxa-7,10,13-triazanonadecanedioic acid (4 mg, 5.7 μmol) and HATU (4.8 mg, 13 μmol) in DMF (2 mL) was added DIEA (10 μL, 57 μmol). After stirring for 45 minutes, 1-(4-((6S,9S,12S)-6-(aminomethyl)-9-isopropyl-2,2-dimethyl-4,7,10-trioxo-12-(3-ureidopropyl)-3-oxa-5,8,11-triazatridecan-13-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (23.7 mg, 13 μmol) was added. After stirring for an additional 1 hour, the solution was diluted with DMSO and purified by ISCO RP-HPLC. Upon lyophilization, 1,1′-(((((2S,5S,8S,32S,35S,38S)-20-(3-(2-((((9H-fluoren-9-yl)methoxy)carbonyl)amino)ethoxy)propanoyl)-8,32-diamino-5,35-diisopropyl-4,7,11,18,22,29,33,36-octaoxo-2,38-bis(3-ureidopropyl)-14,26-dioxa-3,6,10,17,20,23,30,34,37-nonaazanonatriacontanedioyl)bis(azanediyl))bis(4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (16.3 mg, 72% yield) was obtained. HRMS: (M⁺²- H⁺)+=3748.5500, Rt=3.12 min (5 min acidic method).

### Synthesis of 1,1′-(((((2S,5S,8S,32S,35S,38S)-8,32-bis(1-carboxy-3,6,9,12,15,18,21,24,27,30,33,36,39,42,45,48,51,54,57,60,63,66,69,72,75-pentacosaoxaoctaheptacontan-78-amido)-20-(1-(2,5-dioxo-2,5-dihydro-1H-pyrrol-1-yl)-15-oxo-3,6,9,12,19-pentaoxa-16-azadocosan-22-oyl)-5,35-diisopropyl-4,7,11,18,22,29,33,36-octaoxo-2,38-bis(3-ureidopropyl)-14,26-dioxa-3,6,10,17,20,23,30,34,37-nonaazanonatriacontanedioyl)bis(azanediyl))bis(4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L3-P1

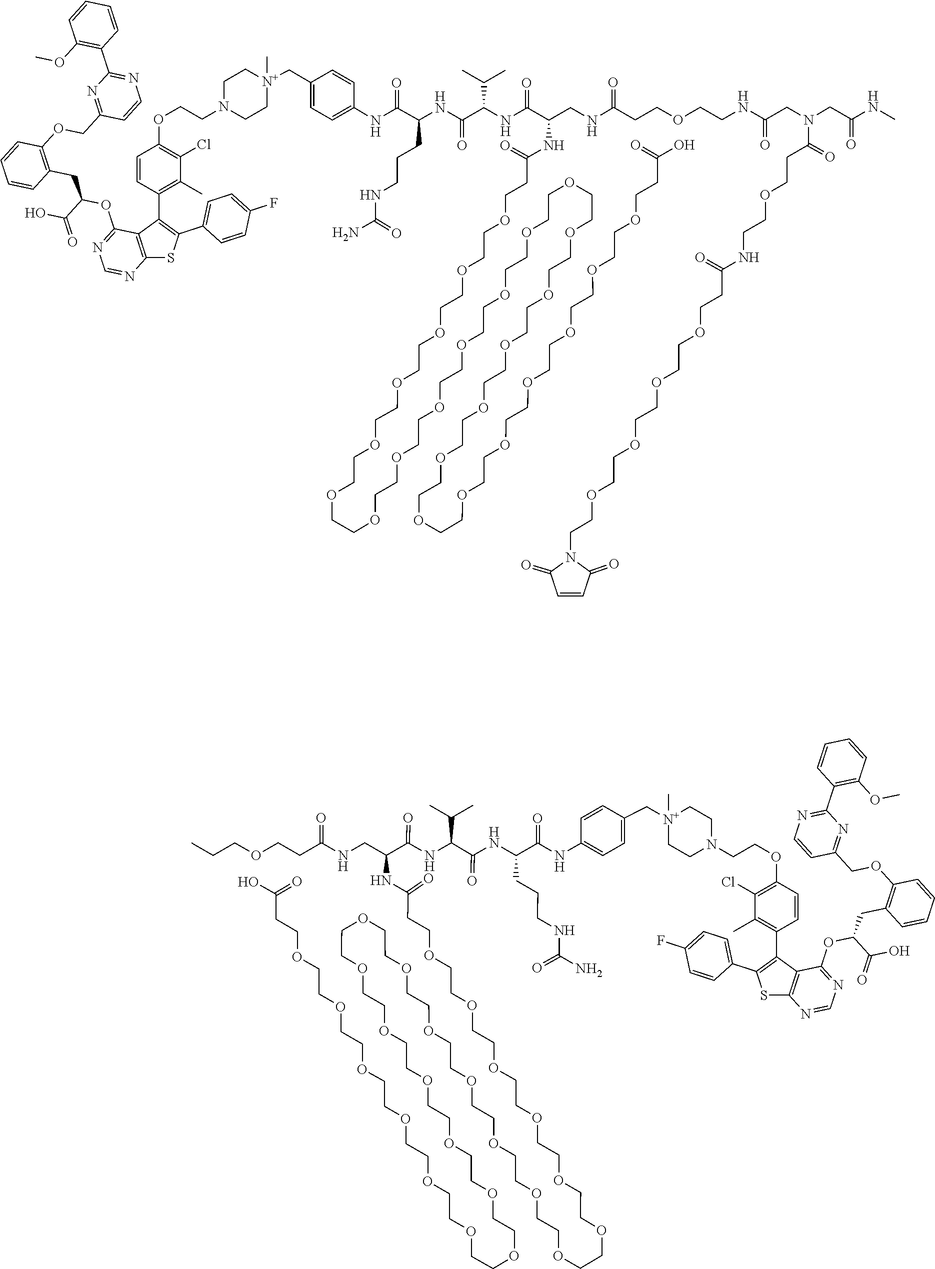

1,1′-(((((2S,5S,8S,32S,35S,38S)-20-(3-(2-((((9H-fluoren-9-yl)methoxy)carbonyl)amino)ethoxy)propanoyl)-8,32-diamino-5,35-diisopropyl-4,7,11,18,22,29,33,36-octaoxo-2,38-bis(3-ureidopropyl)-14,26-dioxa-3,6,10,17,20,23,30,34,37-nonaazanonatriacontanedioyl)bis(azanediyl))bis(4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[l2,3-d]pyrimidin-5-yl)-2-chloro-3-methylphenoxy)ethyl)-1-methylpiperazin-1-ium) trifluoroacetate (16.3 mg, 4.1 μmol) was treated with 33% TFA/CH₂Cl₂ (3 mL) for 1 hour at which time the volatiles were removed in vaculo. The residue was dissolved in MeCN/H₂O and upon lyophilization deprotected material (11.5 mg, 75% yield) was obtained. HRMS: (M⁺²- H⁺)⁺=3308.3401, Rt=2.42 min (5 min acidic method). A solution of the PMB and Boc deprotected material (11.5 mg, 3.05 μmol), 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (10.4 mg, 7.9 μmol) and DIEA (10.7 μL, 61 μmol) in DMF (1 mL) was stirred for 18 hours at which time 2.0 M dimethyl amine in MeOH (31 μL, 61 μmol) was added. After stirring for an additional one hour, the solution was diluted with DMSO and purified by ISCO RP-HPLC. Upon lyophilization, the bispegylated FMOC deprotected material (12.4 mg, 67% yield) was obtained. HRMS: (M⁺²- H⁺)+=5487.6201, Rt=2.51 min (5 min acidic method). Following the second part of GENERAL PROCEDURE #5 using bispegylated FMOC deprotected material (12.4 mg, 2.3 μmol), 2,5-dioxopyrrolidin-1-yl 1-(2,5-dioxo-2,5-dihydro-1H-pyrrol-1-yl)-3,6,9,12-tetraoxapentadecan-15-oate (2.5 mg, 5.6 μmol) and DIEA (7.9 p L, 45 μmol), 1,1′-(((((2S,5S,8S,32S,35S,38S)-8,32-bis(1-carboxy-3,6,9,12,15,18,21,24,27,30,33,36,39,42,45,48,51,54,57,60,63,66,69,72,75-pentacosaoxaoctaheptacontan-78-amido)-20-(1-(2,5-dioxo-2,5-dihydro-1H-pyrrol-1-yl)-15-oxo-3,6,9,12,19-pentaoxa-16-azadocosan-22-oyl)-5,35-diisopropyl-4,7,11,18,22,29,33,36-octaoxo-2,38-bis(3-ureidopropyl)-14,26-dioxa-3,6,10,17,20,23,30,34,37-nonaazanonatriacontanedioyl)bis(azanediyl))bis(4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium) trifluoroacetate (10.2 mg, 74% yield) was obtained. HRMS: (M⁺²- H⁺)+=5814.7598, Rt=2.62 min (5 min acidic method).

### Synthesis of 1,1′-(((((2S,5S,38S,41S)-23-(3-(2-aminoethoxy)propanoyl)-5,38-diisopropyl-20,26-dimethyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

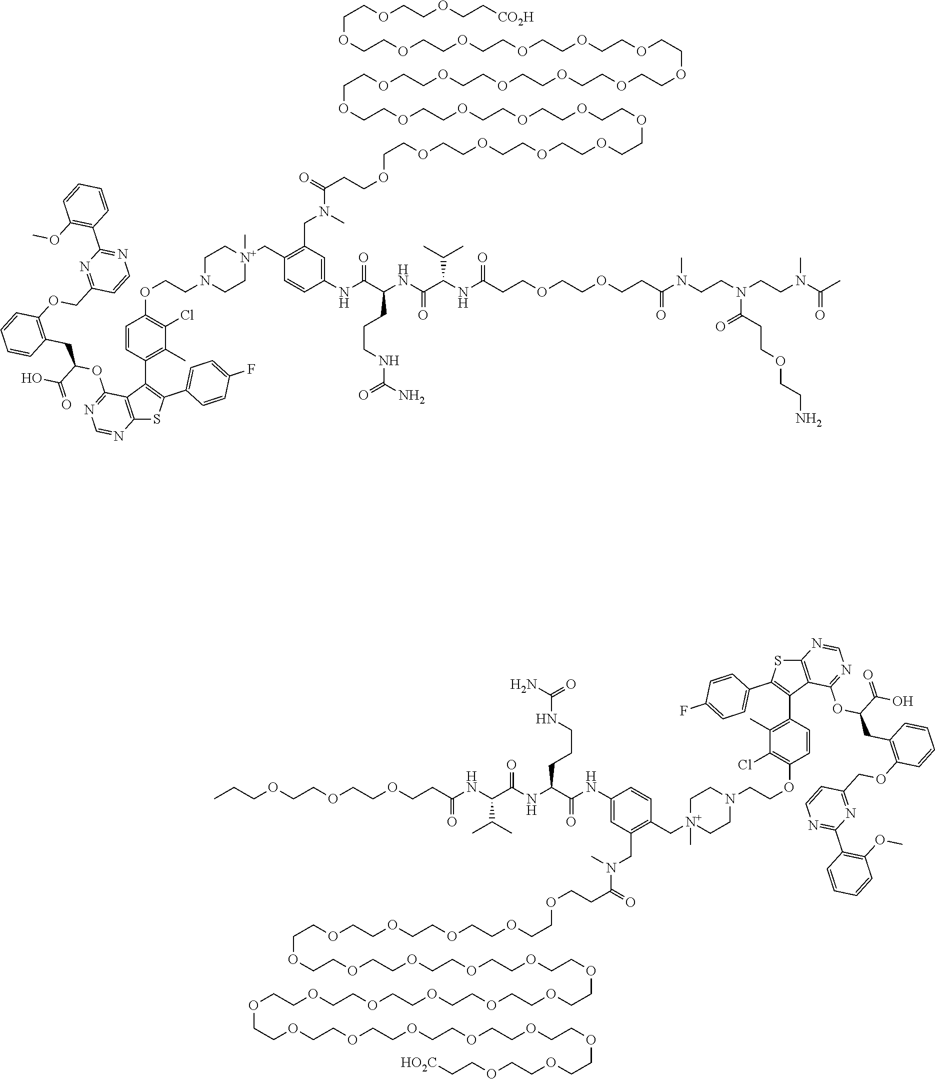

Following the GENERAL PROCEDURE #6 using 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (50.8 mg, 18 μmol), 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioic acid (8 mg, 9 μmol), HATU (6.5 mg, 17 μmol), DIEA (16 μL, 90 μmol) and then 2.0 M Dimethylamine in MeOH (135 μL, 270 μmol), 1,1′-(((((2S,5S,38S,41S)-23-(3-(2-aminoethoxy)propanoyl)-5,38-diisopropyl-20,26-dimethyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (30.6 mg, 56% yield) was obtained. HRMS: (M⁺²- H⁺)+=5589.7998, Rt=2.53 min (5 min acidic method).

### Synthesis of 1,1′-(((((2S,5S,38S,41S)-23-(1-(2,5-dioxo-2,5-dihydro-1H-pyrrol-1-yl)-15-oxo-3,6,9,12,19-pentaoxa-16-azadocosan-22-oyl)-5,38-diisopropyl-20,26-dimethyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L4-P1

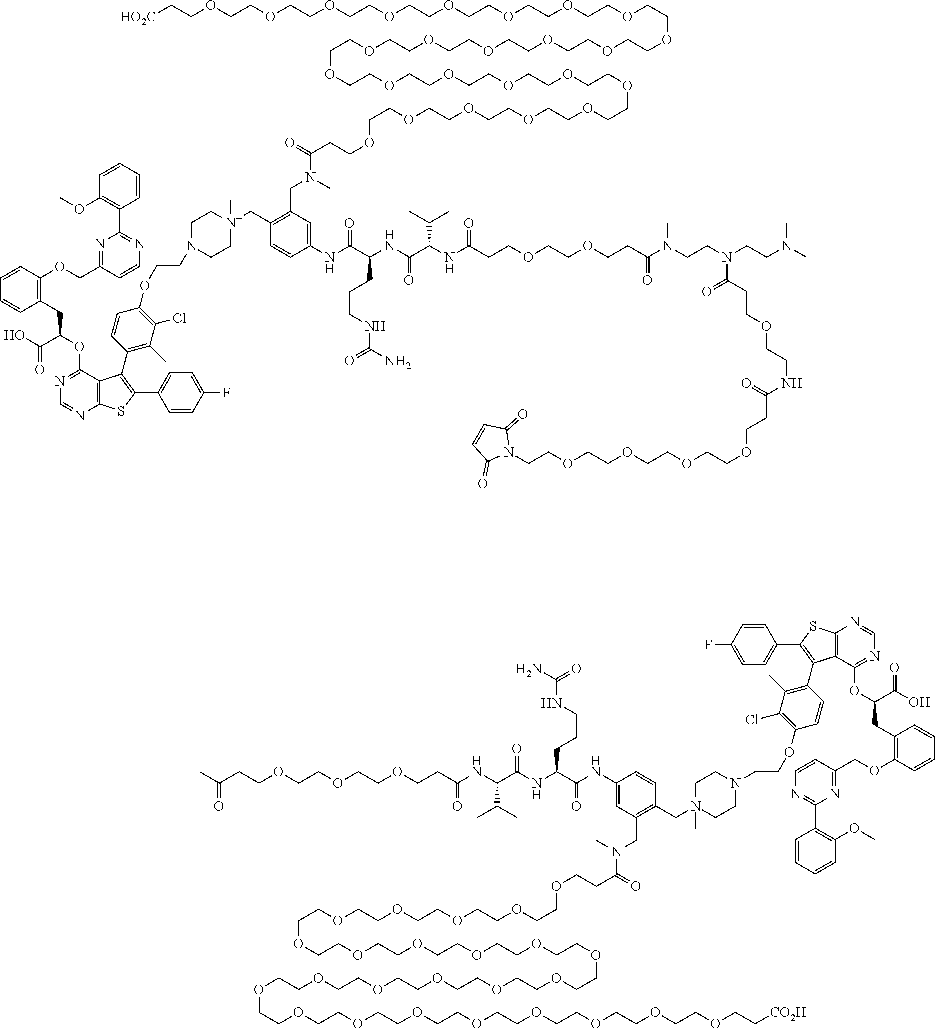

Following the second part of GENERAL PROCEDURE #5 using 1,1′-(((((2S,5S,38S,41S)-23-(3-(2-aminoethoxy)propanoyl)-5,38-diisopropyl-20,26-dimethyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (30.6 mg, 5.1 μmol), 2,5-dioxopyrrolidin-1-yl 1-(2,5-dioxo-2,5-dihydro-1H-pyrrol-l-yl)-3,6,9,12-tetraoxapentadecan-15-oate (6.7 mg, 15 μmol) and DIEA (18 mg, 101 μmol), 1,1′-(((((2S,5S,38S,41S)-23-(1-(2,5-dioxo-2,5-dihydro-1H-pyrrol-1-yl)-15-oxo-3,6,9,12,19-pentaoxa-16-azadocosan-22-oyl)-5,38-diisopropyl-20,26-dimethyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (29.3 mg, 63% yield) was obtained. HRMS: (M⁺²- H⁺)+=5916.8398, Rt=2.65 min (5 min acidic method).

### Synthesis of 1,1′-(((((2S,5S,39S,42S)-22-(3-(2-aminoethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

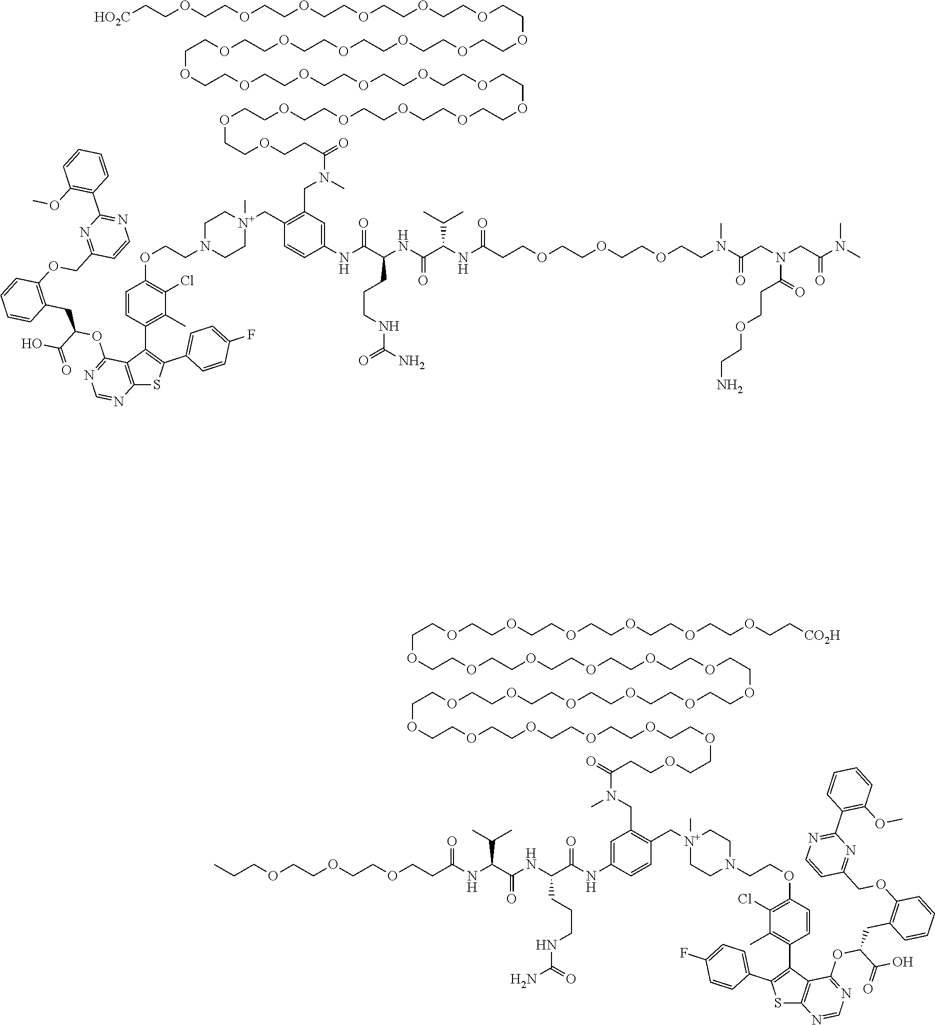

Following the GENERAL PROCEDURE #6 using1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium (52.4 mg, 19 μmol), 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioic acid (8 mg, 8.8 μmol), HATU (6.7 mg, 18 μmol), DIEA (15 μL, 88 μmol) and then 2.0 M Dimethylamine in MeOH (133 μL, 265 μmol), 1,1′-(((((2S,5S,39S,42S)-22-(3-(2-aminoethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (33.6 mg, 63% yield) was obtained. HRMS: (M⁺²- H⁺)+=5605.7700, Rt=2.57 min (5 min acidic method). Synthesis of 1,1′-(((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L5-P1)

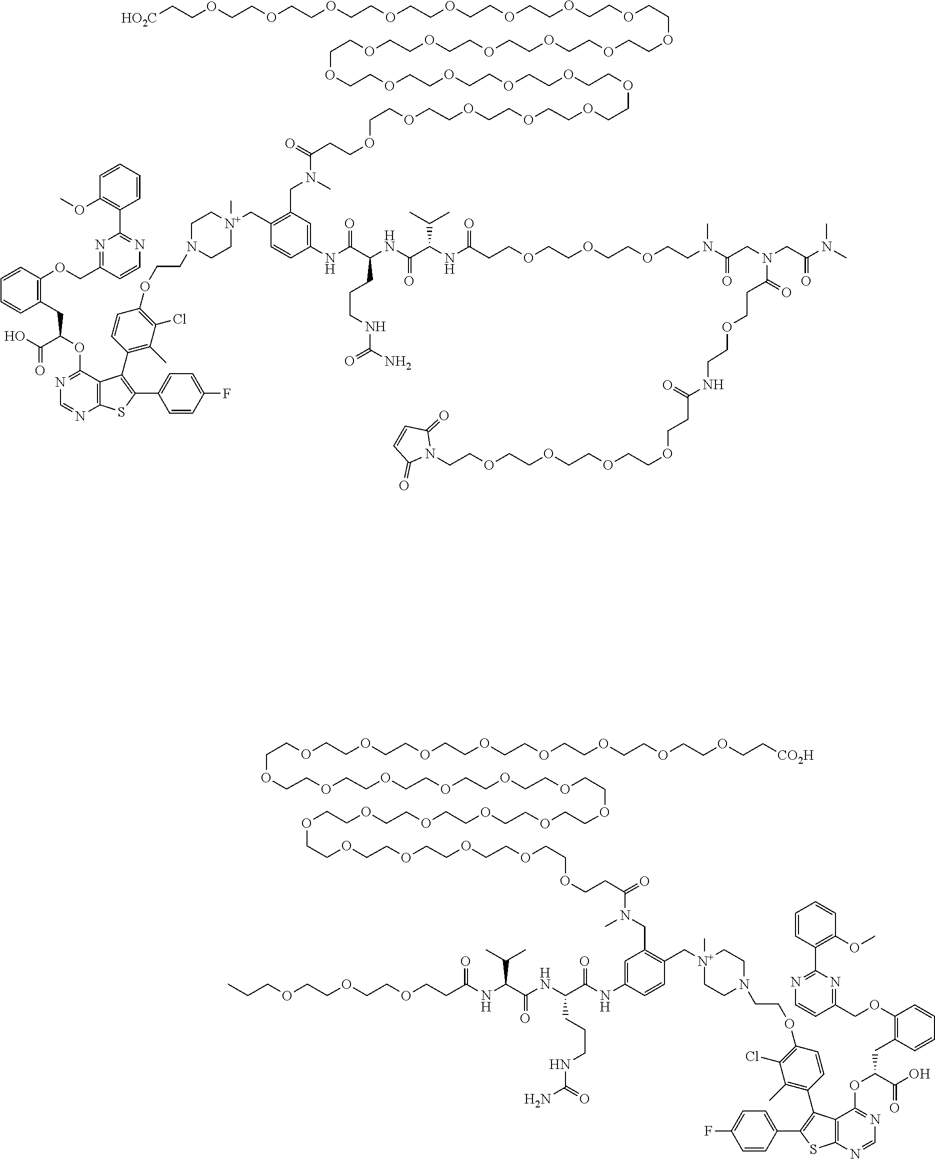

Following the second part of PROCEDURE #5 using 1,1′-(((((2S,5S,39S,42S)-22-(1-(2,5-dioxo-2,5-dihydro-1H-pyrrol-l-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (33.6 mg, 5.5 μmol), 2,5-dioxopyrrolidin-1-yl 1-(2,5-dioxo-2,5-dihydro-1H-pyrrol-l-yl)-3,6,9,12-tetraoxapentadecan-15-oate (7.4 mg, 17 μmol) and DIEA (19 p L, 111 Ipmol), 1,1′-(((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (17.5 mg, 51% yield) was obtained. HRMS: (M⁺²- H⁺)+=5932.8501, Rt=2.68 min (5 min acidic method).

### Synthesis of 1,1′-(((((2S,5S,38S,41S)-23-(3-(2-(amino)ethoxy)propanoyl)-5,38-diisopropyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

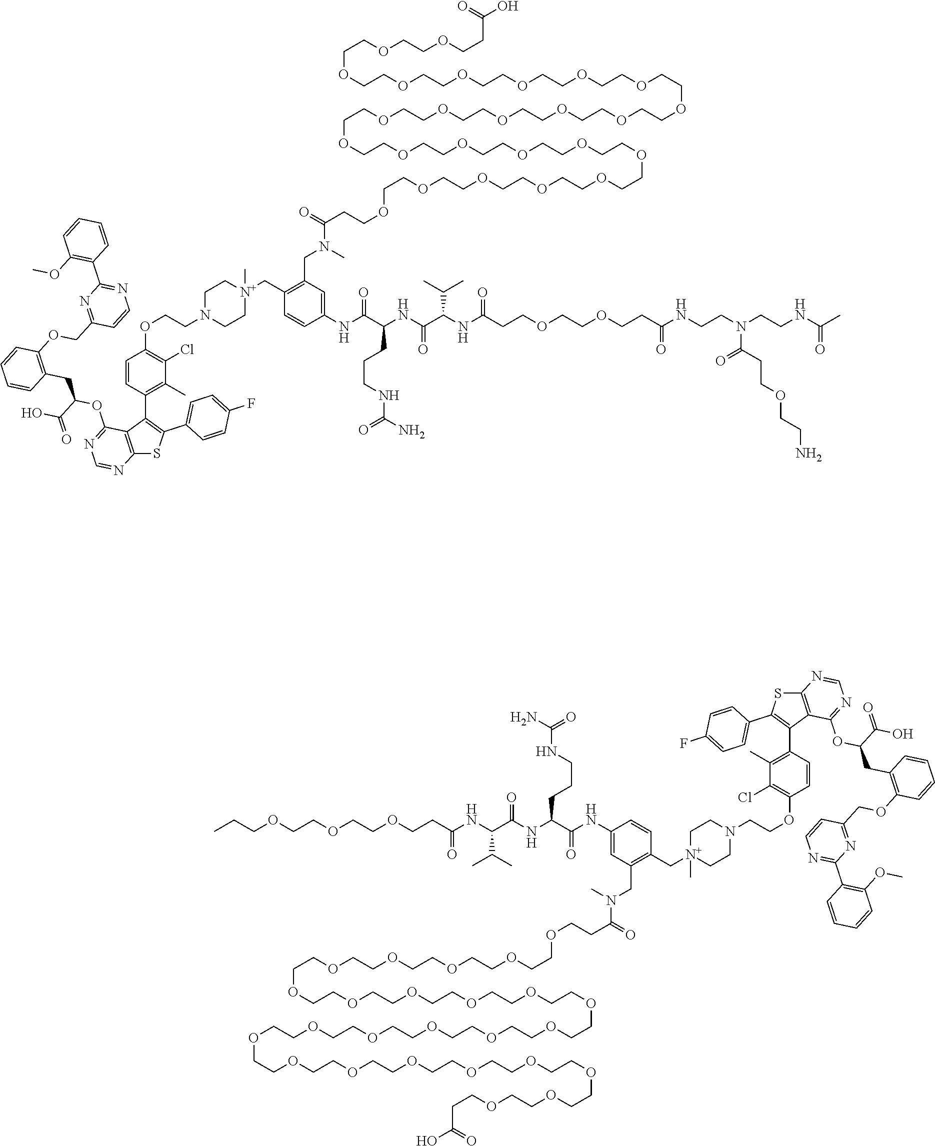

Following the GENERAL PROCEDURE #6 using 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioic acid (11 mg, 12.8 μmol), HATU (9.5 mg, 25 μmol), DIEA (22 μL, 128 μmol) then 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (73.2 mg, 26.3 μmol), and then 2.0 M Dimethylamine in MeOH (190 μL, 380 μmol), 1,1′-(((((2S,5S,38S,41S)-23-(3-(2-(amino)ethoxy)propanoyl)-5,38-diisopropyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (19.6 mg, 25% yield) was obtained. HRMS: (M⁺²- H⁺)+=5561.7402, Rt=2.58 min (5 min acidic method).

### Synthesis of 1,1′-(((((2S,5S,38S,41S)-23-(1-(2,5-dioxo-2,5-dihydro-1H-pyrrol-1-yl)-15-oxo-3,6,9,12,19-pentaoxa-16-azadocosan-22-oyl)-5,38-diisopropyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L6-P1)

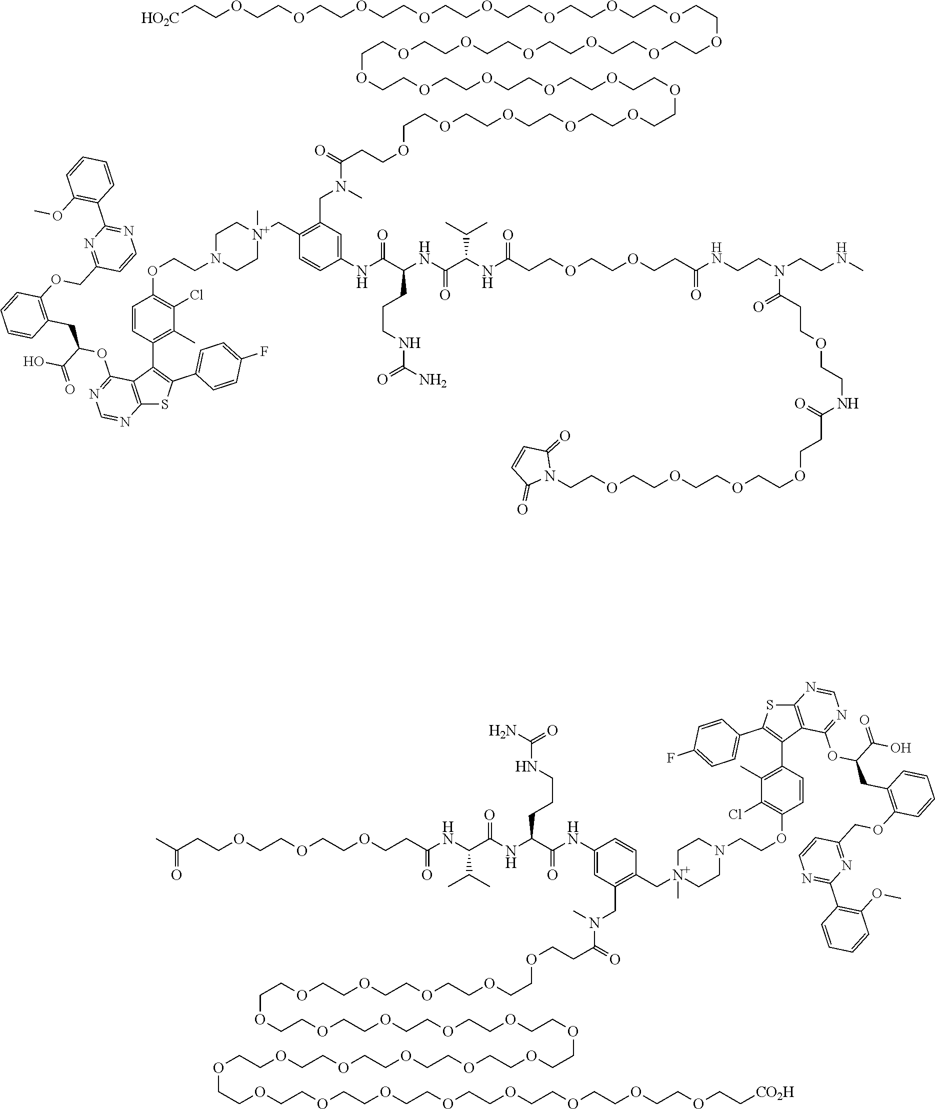

Following the second part of GENERAL PROCEDURE #5 using 1,1′-(((((2S,5S,38S,41S)-23-(3-(2-(amino)ethoxy)propanoyl)-5,38-diisopropyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (19.4 mg, 3.2 μmol), 2,5-dioxopyrrolidin-1-yl 1-(2,5-dioxo-2,5-dihydro-1H-pyrrol-l-yl)-3,6,9,12-tetraoxapentadecan-15-oate (4.3 mg, 9.7 μmol) and DIEA (11 μL, 64 μmol), 1,1′-(((((2S,5S,38S,41S)-23-(1-(2,5-dioxo-2,5-dihydro-1H-pyrrol-l-yl)-15-oxo-3,6,9,12,19-pentaoxa-16-azadocosan-22-oyl)-5,38-diisopropyl-4,7,19,27,36,39-hexaoxo-2,41-bis(3-ureidopropyl)-10,13,16,30,33-pentaoxa-3,6,20,23,26,37,40-heptaazadotetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (11.5 mg, 58% yield) was obtained. HRMS: (M⁺²- H⁺)+=5888.8301, Rt=2.68 min (5 min acidic method).

### Synthesis of 1,1′-(((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L7-P1)

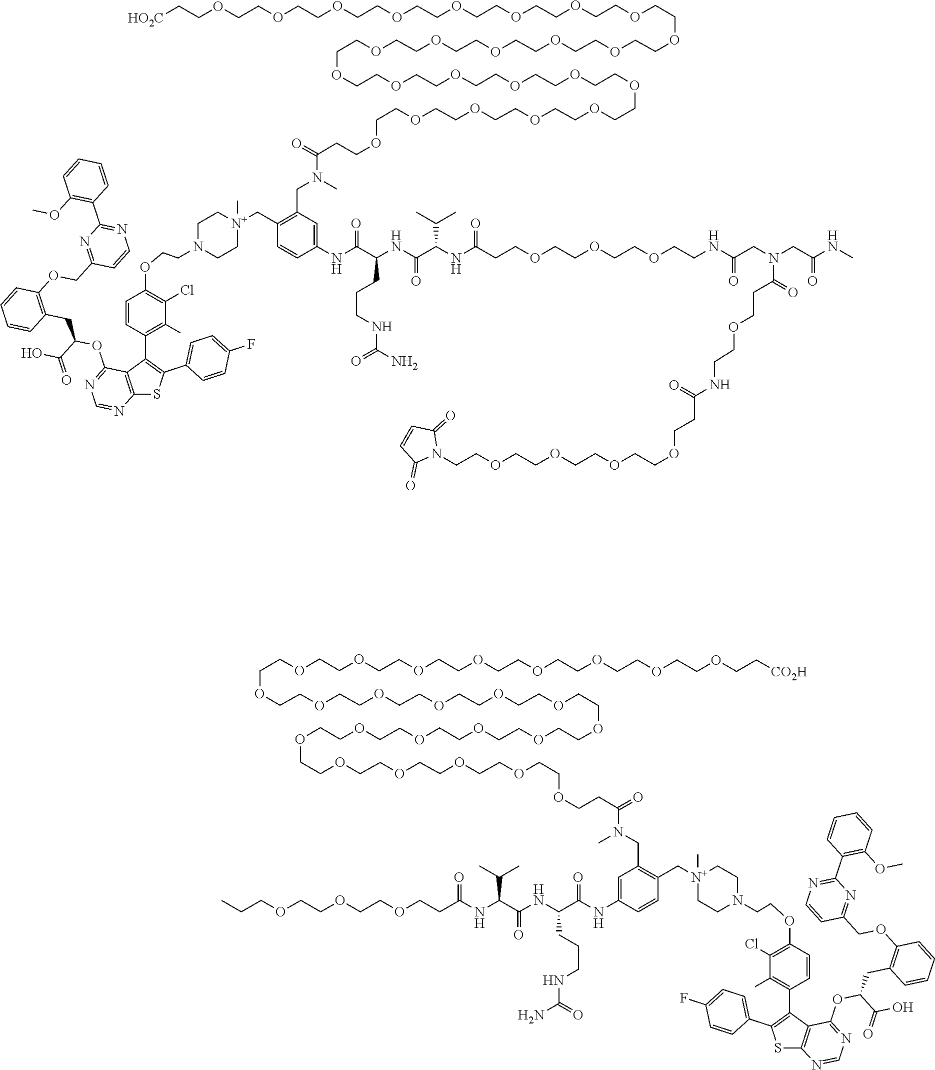

Following the GENERAL PROCEDURE #6 using 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioic acid (10 mg, 11.4 μmol), 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (67.6 mg, 23.8 μmol), HATU (8.7 mg, 22.6 μmol), DIEA (20 p L, 114 μmol) and then 2.0 M Dimethylamine in MeOH (170 μL, 340 μmol), biscoupled product (16.2 mg, 24% yield) was obtained. HRMS: (M⁺²- H⁺)+=5577.7300, Rt=2.63 min (5 min acidic method). Following the second part of GENERAL PROCEDURE #5 and using isolated biscoupled amine (16.2 mg, 2.7 μmol), 2,5-dioxopyrrolidin-1-yl 1-(2,5-dioxo-2,5-dihydro-1H-pyrrol-1-yl)-3,6,9,12-tetraoxapentadecan-15-oate (1.8 mg, 4.5 μmol) and DIEA (4.7 μL, 27 μmol), 1,1′-(((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (10.6 mg, 64% yield) was obtained. HRMS: (M⁺²- H⁺)⁺=5904.8308, Rt=2.53 min (5 min acidic method).

### Synthesis of 1-(4-((6S,9S,37S,40S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(80-carboxy-2-methyl-3-oxo-5,9,12,15,18,21,24,27,30,33,36,39,42,45,48,51,54,57,60,63,66,69,72,75,78-pentacosaoxa-2-azaoctacontyl)phenyl)carbamoyl)-23-(1-(2,5-dioxo-2,5-dihydro-1H-pyrrol-1-yl)-15-oxo-3,6,9,12,19-pentaoxa-16-azadocosan-22-oyl)-9,37-diisopropyl-1,8,11,35,38-pentaoxo-40-(3-ureidopropyl)-14,17,20,26,29,32-hexaoxa-2,7,10,23,36,39-hexaazahentetracontan-41-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L9-P1)

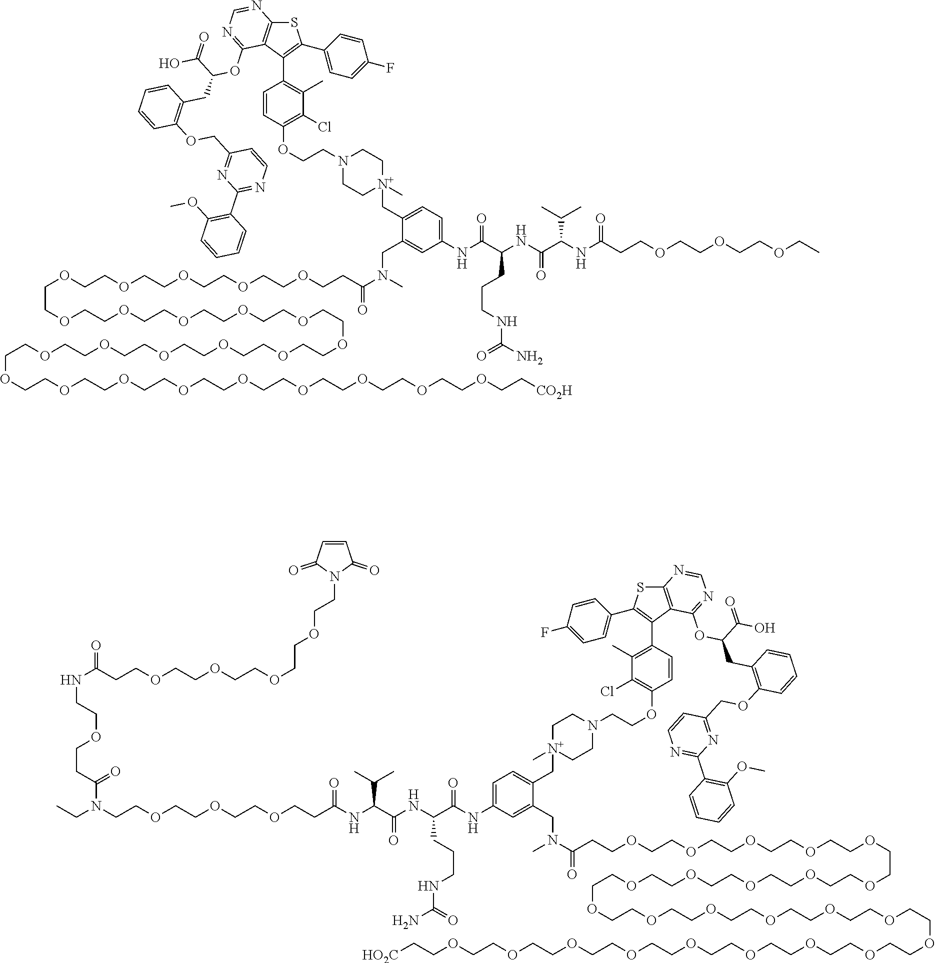

Following the GENERAL PROCEDURE #6 using 13-(3-(2-((((9H-fluoren-9-yl)methoxy)carbonyl)amino)ethoxy)propanoyl)-4,7,10,16,19,22-hexaoxa-13-azapentacosanedioic acid (15 mg, 20 μmol), 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (107 mg, 39 μmol), HATU (14.6 mg, 38 μmol), DIEA (52 μL, 295 μmol) and then 2.0 M Dimethylamine in MeOH (295 μL, 590 μmol), biscoupled amine product (38 mg, 32% yield) was obtained. HRMS: (M⁺²- H⁺)+=5463.6802, Rt=2.53 min (5 min acidic method). Following the second part of GENERAL PROCEDURE #5 using biscoupled amine product (38 mg, 6.5 μmol), 2,5-dioxopyrrolidin-1-yl 1-(2,5-dioxo-2,5-dihydro-1H-pyrrol-1-yl)-3,6,9,12-tetraoxapentadecan-15-oate (4.3 mg, 9.8 μmol) and DIEA (17 μL, 98 μmol), 1-(4-((6S,9S,37S,40S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium-1-yl)methyl)-3-(80-carboxy-2-methyl-3-oxo-5,9,12,15,18,21,24,27,30,33,36,39,42,45,48,51,54,57,60,63,66,69,72,75,78-pentacosaoxa-2-azaoctacontyl)phenyl)carbamoyl)-23-(1-(2,5-dioxo-2,5-dihydro-1H-pyrrol-l-yl)-15-oxo-3,6,9,12,19-pentaoxa-16-azadocosan-22-oyl)-9,37-diisopropyl-1,8,11,35,38-pentaoxo-40-(3-ureidopropyl)-14,17,20,26,29,32-hexaoxa-2,7,10,23,36,39-hexaazahentetracontan-41-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (30 mg, 74% yield) was obtained. HRMS: (M⁺²- H⁺)+=5790.7798, Rt=2.54 min (5 min acidic method).

### Synthesis of 1-(4-((6S,9S,43S,46S)-1-amino-26-(3-(2-aminoethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)phenyl)carbamoyl)-9,43-diisopropyl-1,8,11,41,44-pentaoxo-46-(3-ureidopropyl)-14,17,20,23,29,32,35,38-octaoxa-2,7,10,26,42,45-hexaazaheptatetracontan-47-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-(2-(2-(2-methoxyphenyl)pyrimidin-4-yl)ethyl)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

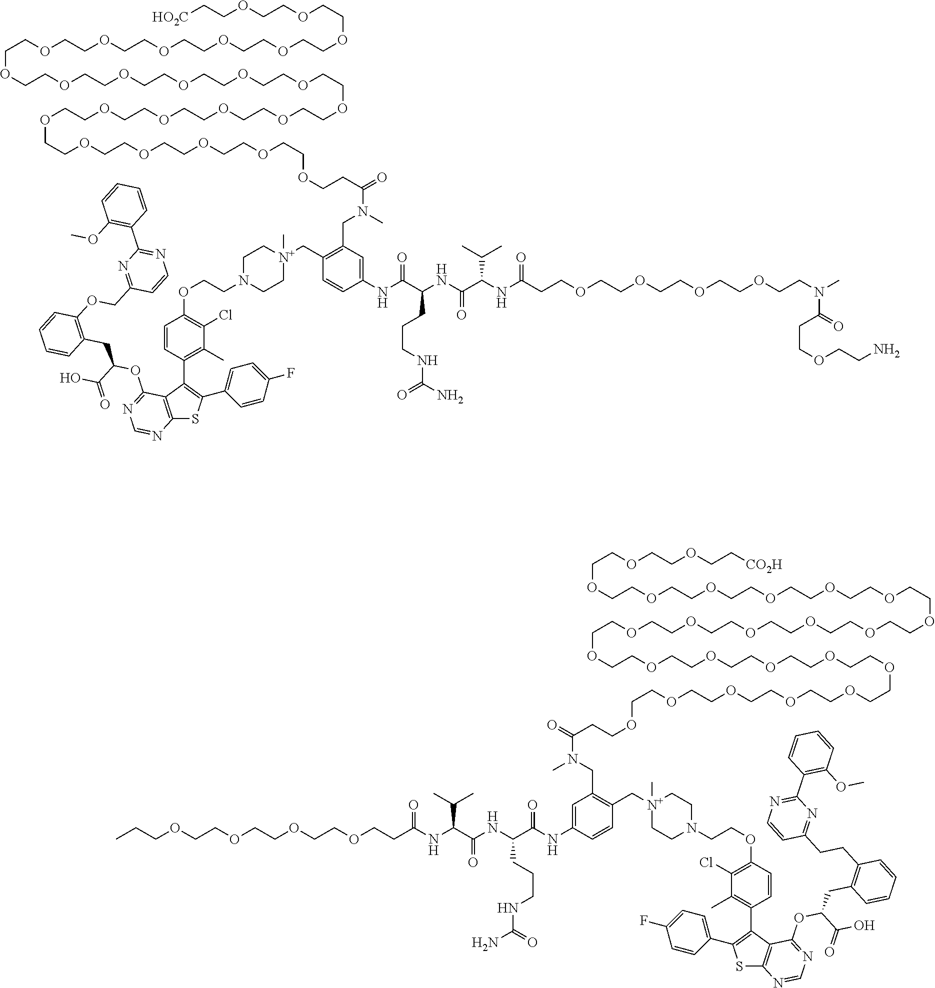

To a solution of 16-(3-(2-((tert-butoxycarbonyl)amino)ethoxy)propanoyl)-4,7,10,13,19,22,25,28-octaoxa-16-azahentriacontanedioic acid (10.9 mg, 15 μmol) and HATU (11 mg, 30 μmol) in DMF (2 mL) was added DIEA (52 μL, 298 μmol). After stirring for 15 minutes, 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (84 mg, 31 μmol) was added. After stirring for an additional 3 hours, the solution was diluted with DMSO and purified by ISCO RP-HPLC. Upon lyophilization, the material was treated with 25% TFA/CH₂Cl₂ (2 mL) for 1 hour at which time the volatiles were removed in vacuo. After trituration with Et₂O and pumping on high vacuum, 1-(4-((6S,9S,43S,46S)-1-amino-26-(3-(2-aminoethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium-1-yl)methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)phenyl)carbamoyl)-9,43-diisopropyl-1,8,11,41,44-pentaoxo-46-(3-ureidopropyl)-14,17,20,23,29,32,35,38-octaoxa-2,7,10,26,42,45-hexaazaheptatetracontan-47-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-(2-(2-(2-methoxyphenyl)pyrimidin-4-yl)ethyl)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (31 mg, 35% yield) was obtained. HRMS: (M⁺²- H⁺)+=5549.7798, Rt=2.54 min (5 min acidic method).

### Synthesis of 1-(4-((6S,9S,43S,46S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-1,8,11,41,44-pentaoxo-46-(3-ureidopropyl)-14,17,20,23,29,32,35,38-octaoxa-2,7,10,26,42,45-hexaazaheptatetracontan-47-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-(2-(2-(2-methoxyphenyl)pyrimidin-4-yl)ethyl)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L10-P1)

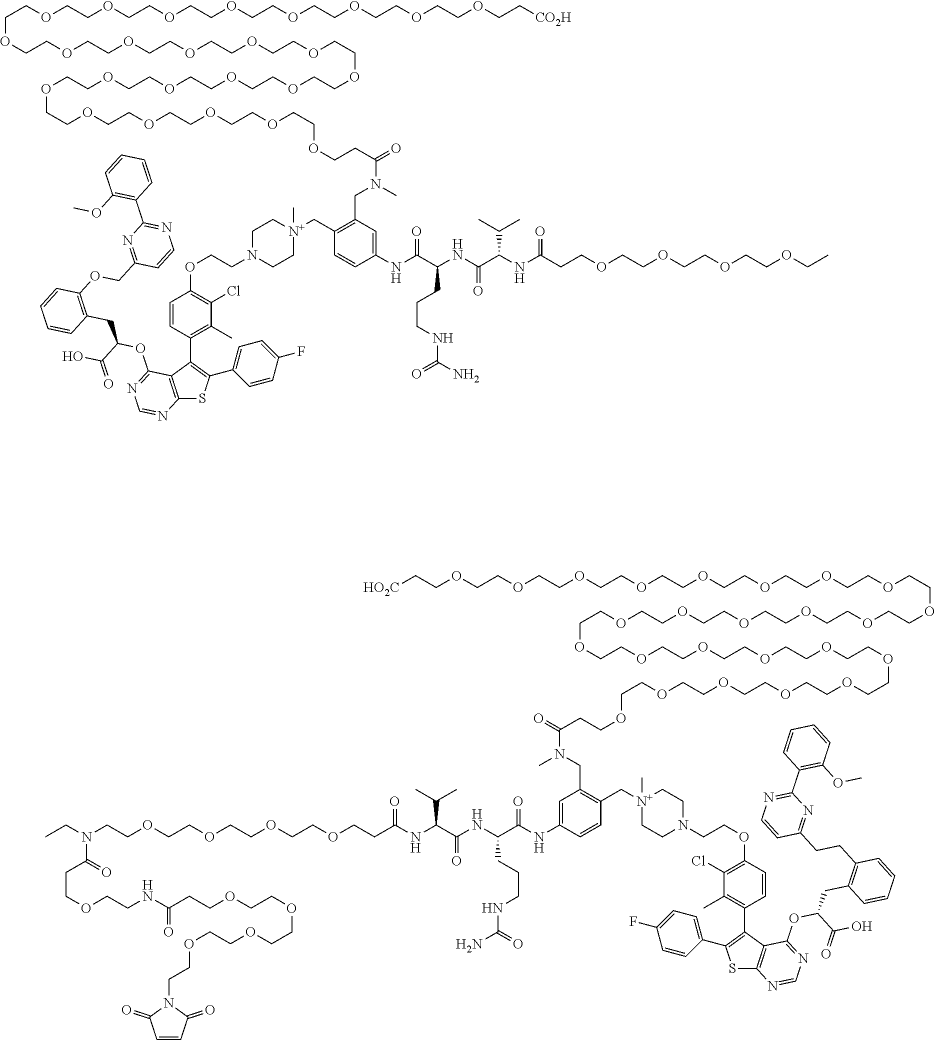

Following the second part of GENERAL PROCEDURE #5 using 1-(4-((6S,9S,43S,46S)-1-amino-26-(3-(2-aminoethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)phenyl)carbamoyl)-9,43-diisopropyl-1,8,11,41,44-pentaoxo-46-(3-ureidopropyl)-14,17,20,23,29,32,35,38-octaoxa-2,7,10,26,42,45-hexaazaheptatetracontan-47-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-(2-(2-(2-methoxyphenyl)pyrimidin-4-yl)ethyl)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (31 mg, 5.3 μmol), 2,5-dioxopyrrolidin-1-yl 1-(2,5-dioxo-2,5-dihydro-1H-pyrrol-l-yl)-3,6,9,12-tetraoxapentadecan-15-oate (7 mg, 16 μmol) and DIEA (15 μL, 86 μmol), 1-(4-((6S,9S,43S,46S)-1-amino-6-((4-((4-(2-(4-(4-((R)-1-carboxy-2-(2-((2-(2- methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)phenyl)carbamoyl)-26-(1-(2,5-dioxo-2,5-dihydro-1H-pyrrol-l-yl)-15-oxo-3,6,9,12,19-pentaoxa-16-azadocosan-22-oyl)-9,43-diisopropyl-1,8,11,41,44-pentaoxo-46-(3-ureidopropyl)-14,17,20,23,29,32,35,38-octaoxa-2,7,10,26,42,45-hexaazaheptatetracontan-47-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-(2-(2-(2-methoxyphenyl)pyrimidin-4-yl)ethyl)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (20.8 mg, 66% yield) was obtained. HRMS: (M⁺²- H⁺)+=5877.8198, Rt=2.67 min (5 min acidic method).

### Synthesis of 1-({4-[(2S,5S,37S,40S)-45-Amino-2-(3-carbamamidopropyl)-40-{[4-({4-[2-(4-{4-[(1R)-1- carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-19-[1-(2,5-dioxo-2,5-dihydro-1H-pyrrol-1-yl)-15,22-dioxo-3,6,9,12,19-pentaoxa-16-azadocosan-22-yl]-4,7,23,25,25,35,38,45-octaoxo-5,37-di(propan-2-yl)-10,13,16,22,29,32-hexaoxa-25k6-thia-3,6,19,24,26,36,39,44-octaazapentatetracontanan-1-amido]-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl}methyl)-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidinGP-1o64,c1 5-yl}-2-chloro-3-methylphenoxy)ethyl]-1-methylpiperazin-1-ium trifluoroacetate (P1-Li1-P1)

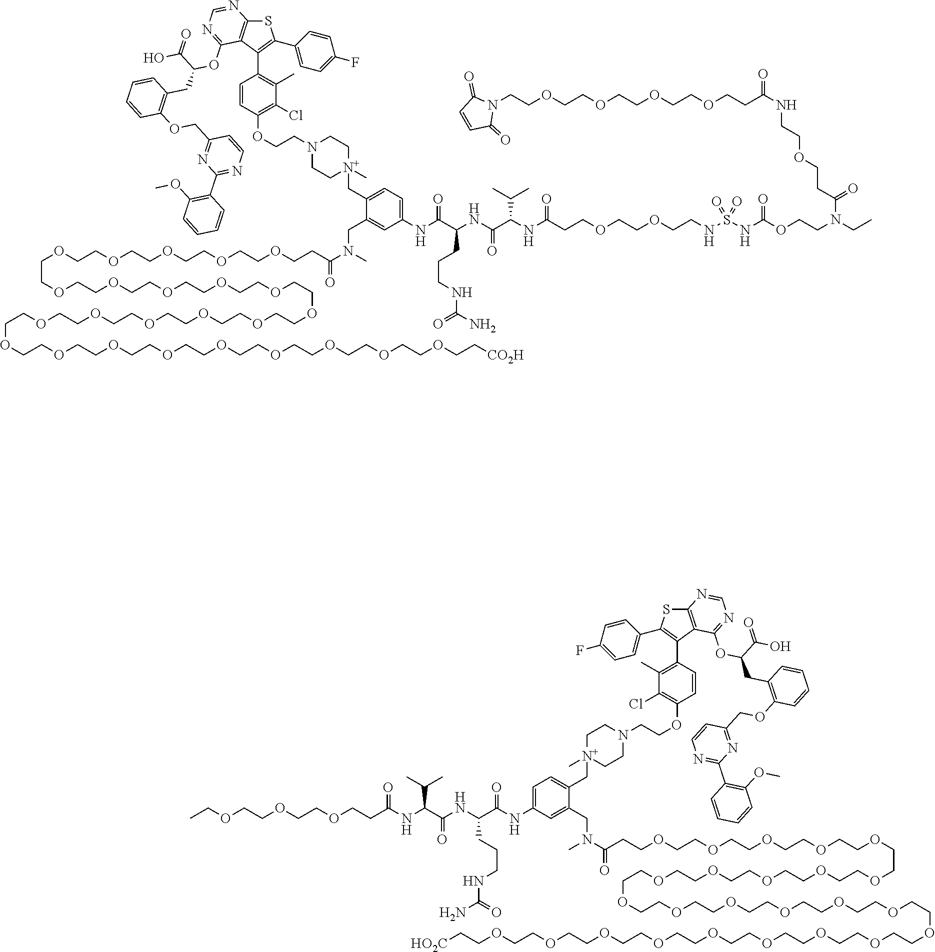

To a solution of 11-(2-(((N-(2-(2-(2-carboxyethoxy)ethoxy)ethyl)sulfamoyl)carbamoyl)oxy)ethyl)-1-(9H-fluoren-9-yl)-3,10-dioxo-2,7,14,17,20-pentaoxa-4,11-diazatricosan-23-oic acid (14.8 mg, 17 μmol) and HATU (12.7 mg, 33 μmol) in DMF (2 mL) was added DIEA (44 μL, 251 μmol). After stirring for 30 minutes, 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (103 mg, 35 μmol) was added. After stirring for an additional 1 hour, 2M Me₂NH in MeOH (251 μL, 502 pM) was added. After stirring for one additional hour, the solution was diluted with DMSO and purified by ISCO RP-HPLC. Upon lyophilization, bisamide coupled, FMOC deprotected product (33.4 mg, 33% yield) was obtained. HRMS: (M⁺²- H⁺)+=5585.6499, Rt=2.52 min (5 min acidic method). Following second half of GENERAL PROCEDURE #5 using above isolated bisamide coupled, FMOC deprotected product (33.4 mg, 5.6 μmol), 2,5-dioxopyrrolidin-1-yl 1-(2,5-dioxo-2,5-dihydro-1H-pyrrol-1-yl)-3,6,9,12-tetraoxapentadecan-15-oate (3.7 mg, 8.4 μmol) and DIEA (9.8 p L, 56 μmol), 1-({4-[(2S,5S,37S,40S)-45-Amino-2-(3-carbamamidopropyl)-40-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-19-[1-(2,5-dioxo-2,5-dihydro-1H-pyrrol-1-yl)-15,22-dioxo-3,6,9,12,19-pentaoxa-16-azadocosan-22-yl]-4,7,23,25,25,35,38,45-octaoxo-5,37-di(propan-2-yl)-10,13,16,22,29,32-hexaoxa-25,6-thia-3,6,19,24,26,36,39,44-octaazapentatetracontanan-1-amido]-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl}methyl)-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (18.5 mg, 53% yield) was obtained. HRMS: (M⁺²- H⁺)+=5912.8091, Rt=2.65 min (5 min acidic method).

### Synthesis of N-(4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

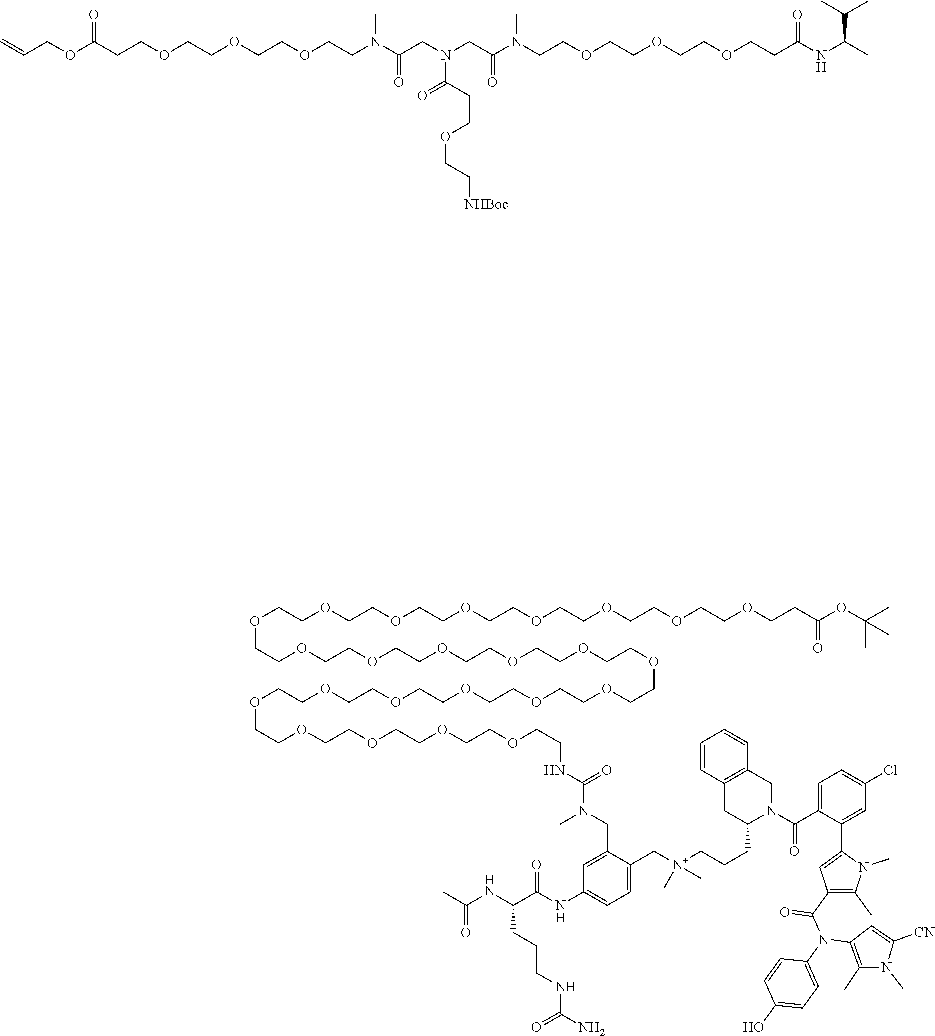

Following GENERAL PROCEDURE #3 using N-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (110 mg, 43 μmol) and 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (42.3 mg, 51 μmol), N-(4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (134.3 mg, 96%) was obtained. HRMS: M⁺=3139.7400, Rt=2.59 min (5 min acidic method).

### Synthesis of N-(4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

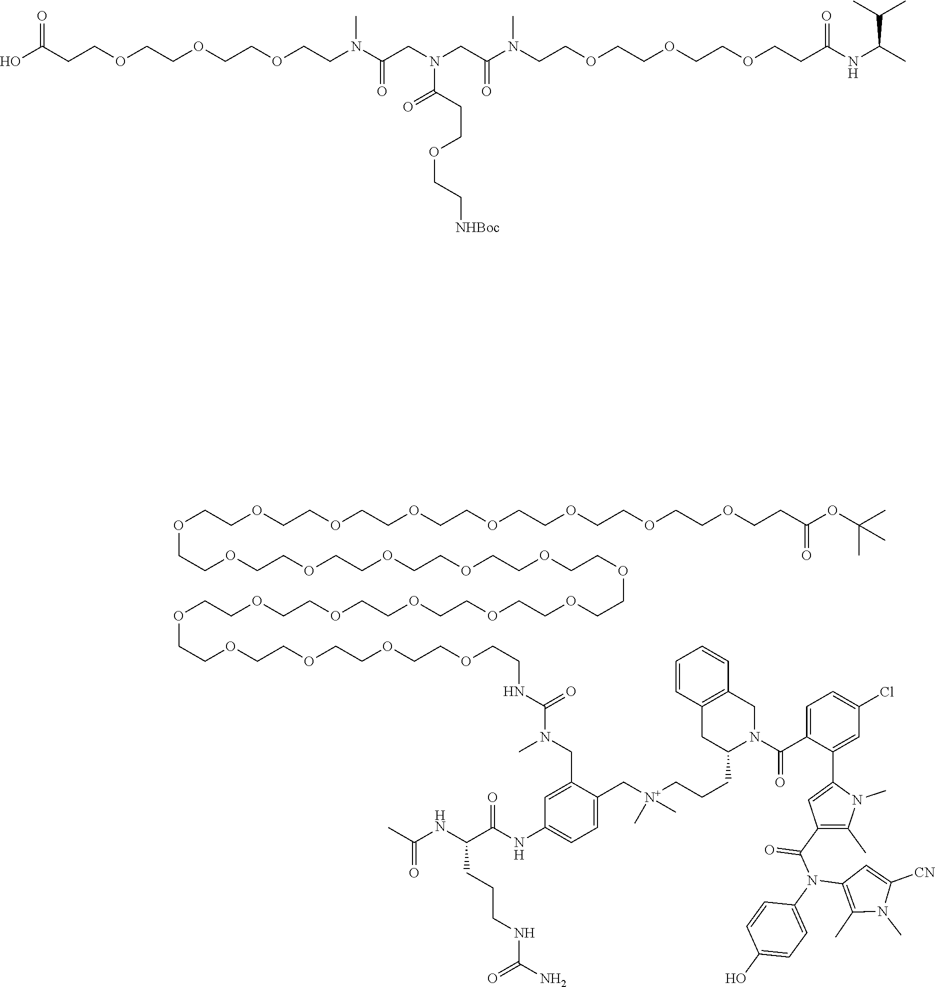

Following GENERAL PROCEDURE #2 using N-(4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (227 mg, 70 μmol), N,N,1,1,1-pentamethylsilanamine (82 mg, 700 μmol) and tetrakis(triphenylphosphine)palladium (12.1 mg, 10.5 μmol), N-(4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (178.3 mg, 79% yield) was obtained. HRMS: M⁺=3099.700, Rt=2.46 min (5 min acidic method).

### Synthesis of 1-(4-((6S,9S,43S,46S)-1-amino-26-(3-(2-((tert-butoxycarbonyl)amino)ethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

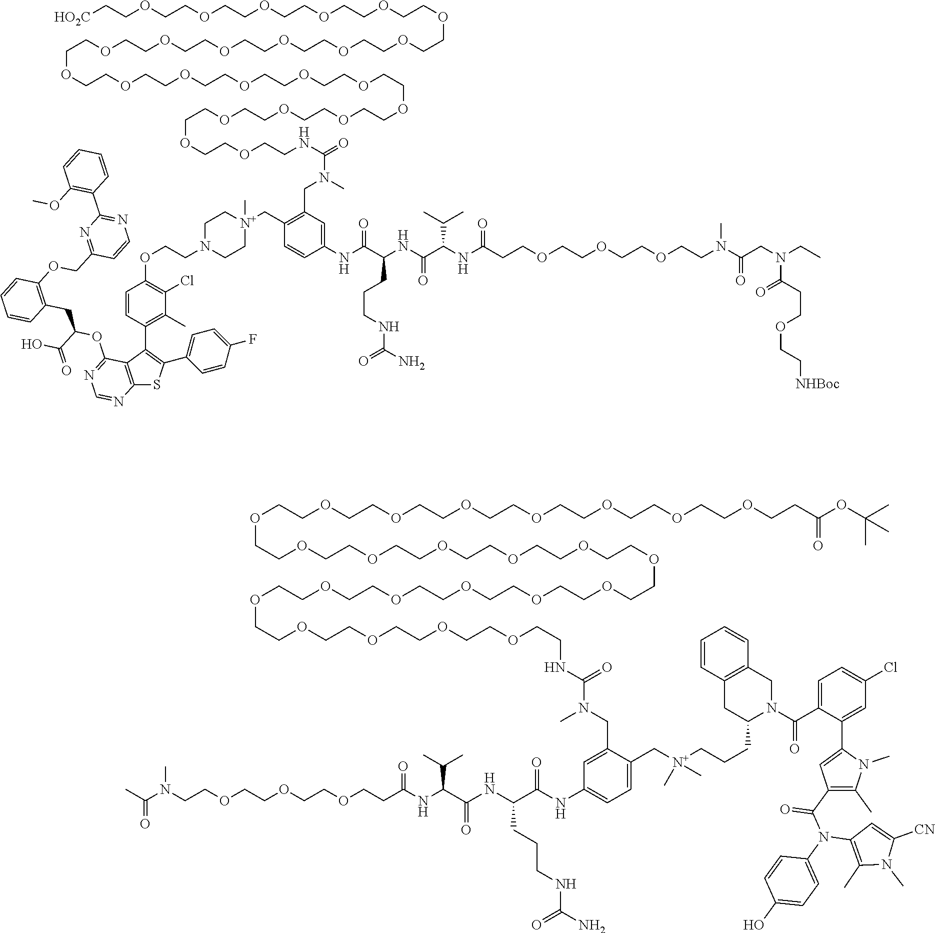

Following GENERAL PROCEDURE #4 using N-(4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (118 mg, 37 μmol), TSTU (11.8 mg, 39 μmol), 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium TFA salt (108 mg, 40 μmol) and DIEA (33.2 mg, 257 μmol), 1-(4-((6S,9S,43S,46S)-1-amino-26-(3-(2-((tert-butoxycarbonyl)amino)ethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (101 mg, 48% yield) was obtained. HRMS: (M⁺²-H⁺)+=5531.8799, Rt=2.65 min (5 min acidic method).

### Synthesis of 1-(4-((6S,9S,43S,46S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifuoroacetate (P1-L12-P2)

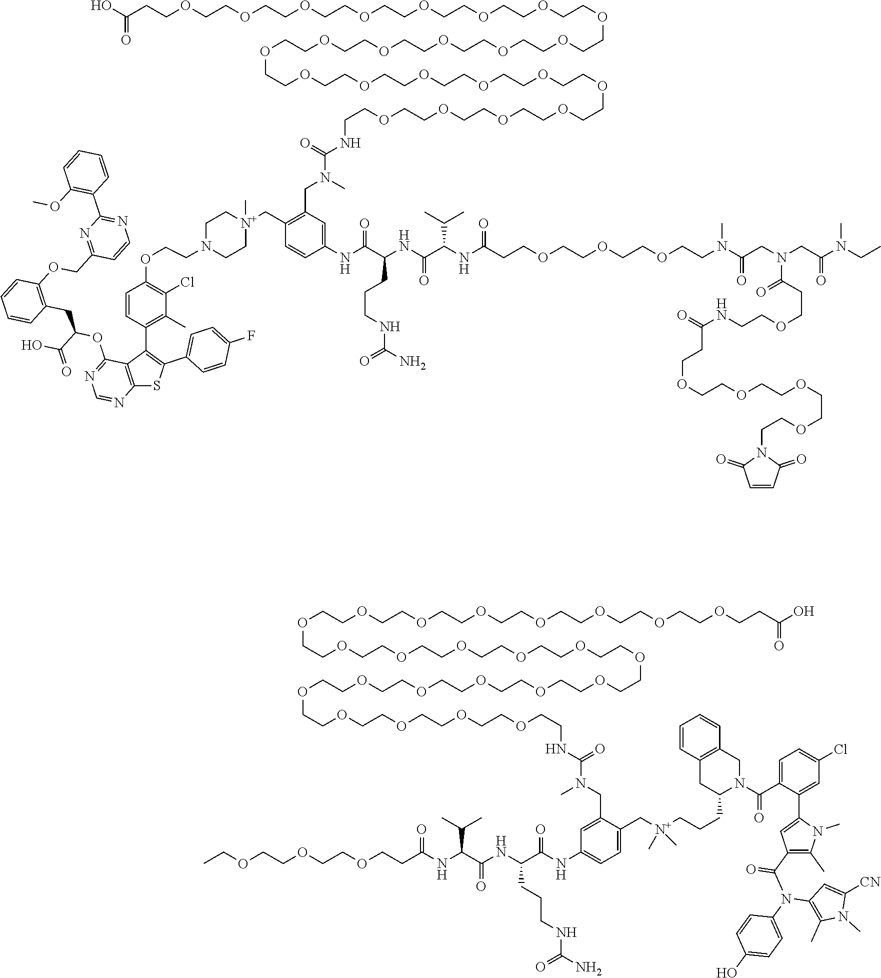

Following the first part of GENERAL PROCEDURE #5 and using 1-(4-((6S,9S,43S,46S)-1-amino-26-(3-(2-((tert-butoxycarbonyl)amino)ethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (214 mg, 35 μmol), after removal of volatiles in vacuo and purification by RP-HPLC the deprotected amine intermediate was isolated. Following the second part of GENERAL PROCEDURE #5 and using isolated amine (176 mg, 30 μmol), 2,5-dioxopyrrolidin-1-yl 1-(2,5-dioxo-2,5-dihydro-1H-pyrrol-1-yl)-3,6,9,12-tetraoxapentadecan-15-oate (20.2 mg, 46 μmol) and DIEA (31.5 mg, 243 μmol), 1-(4-((6S,9S,43S,46S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-26-(1-(2,5-dioxo-2,5-dihydro-1 H-pyrrol-1-yl)-15-oxo-3,6,9,12,19-pentaoxa-16-azadocosan-22-oyl)-9,43-diisopropyl-23,29-dimethyl-1, 8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifuoroacetate (143 mg, 66% yield) was obtained. HRMS: (M⁺²-H⁺)+=5702.8599, Rt=2.44 min (5 min acidic method).

### Synthesis of 4-(4-((2R,5R)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((S)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

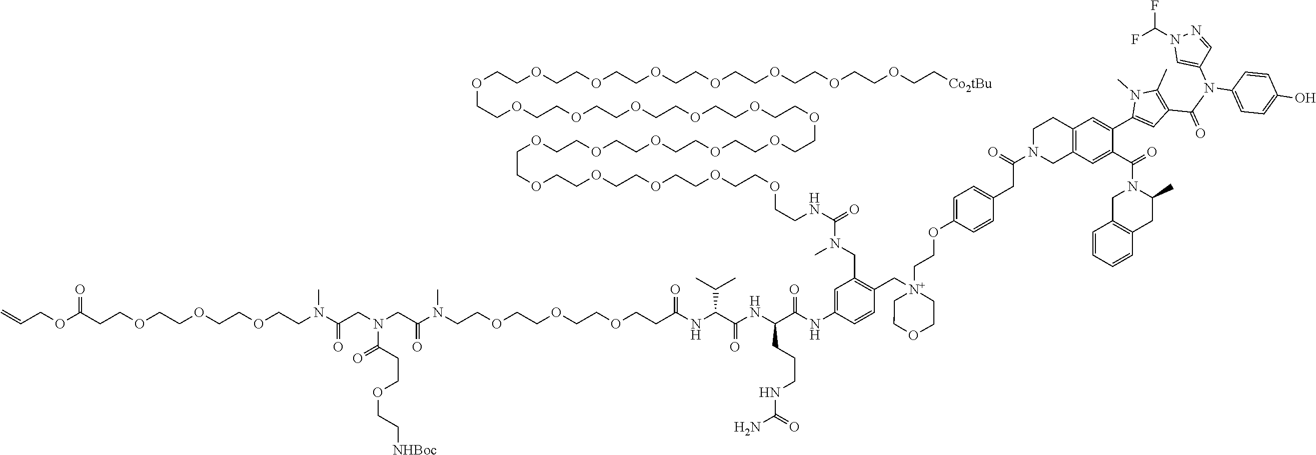

Following GENERAL PROCEDURE #3 using 4-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (300 mg, 118.5 μmol) and 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (112 mg, 136.3 μmol), 4-(4-((2R,5R)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((S)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (256 mg, 65%) was obtained. HRMS: M⁺=3334.8201, Rt=2.56 min (5 min acidic method).

### Synthesis of 4-(4-((32R,35R)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((S)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

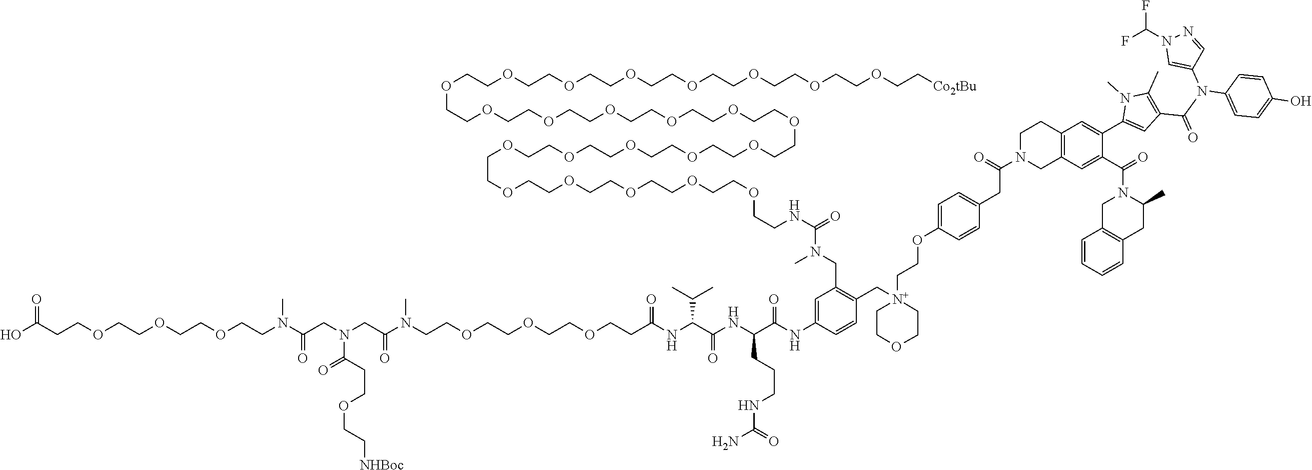

Following PROCEDURE #2 using 4-(4-((2R,5R)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((S)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (256.5 mg, 76.9 μmol), pyrrolidine (38.3 mg, 538 μmol) and tetrakis(triphenylphosphine)palladium (8.8 mg, 7.7 μmol) was, 4-(4-((32R,35R)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((S)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (171 mg, 67% yield) was obtained. HRMS: M⁺=3294.8000 Rt=2.45 min (5 min acidic method).

### Synthesis of 4-(4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

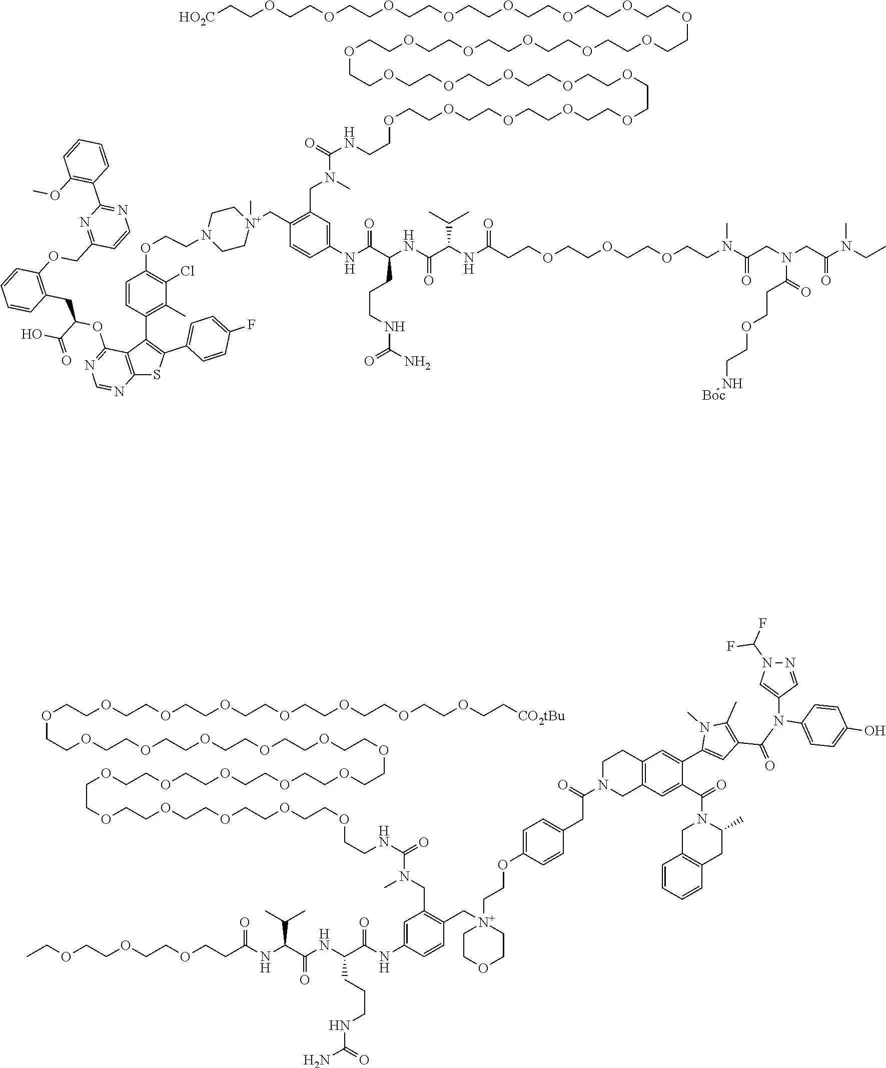

Following GENERAL PROCEDURE #4 using 4-(4-((32R,35R)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((S)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (170.4 mg, 51.7 μmol), TSTU (15.6 mg, 51.7 μmol), 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium TFA salt (149.2 mg, 59.4 μmol) and DIEA (72 μL, 414 μmol), 4-(4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (147 mg, 49.6% yield) was obtained. HRMS: (M⁺²-H⁺)+=5726.9800, Rt=2.66 min (5 min acidic method).

### Synthesis of 4-(4-((6S,9S,43S,46S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (P1-L12-P3)

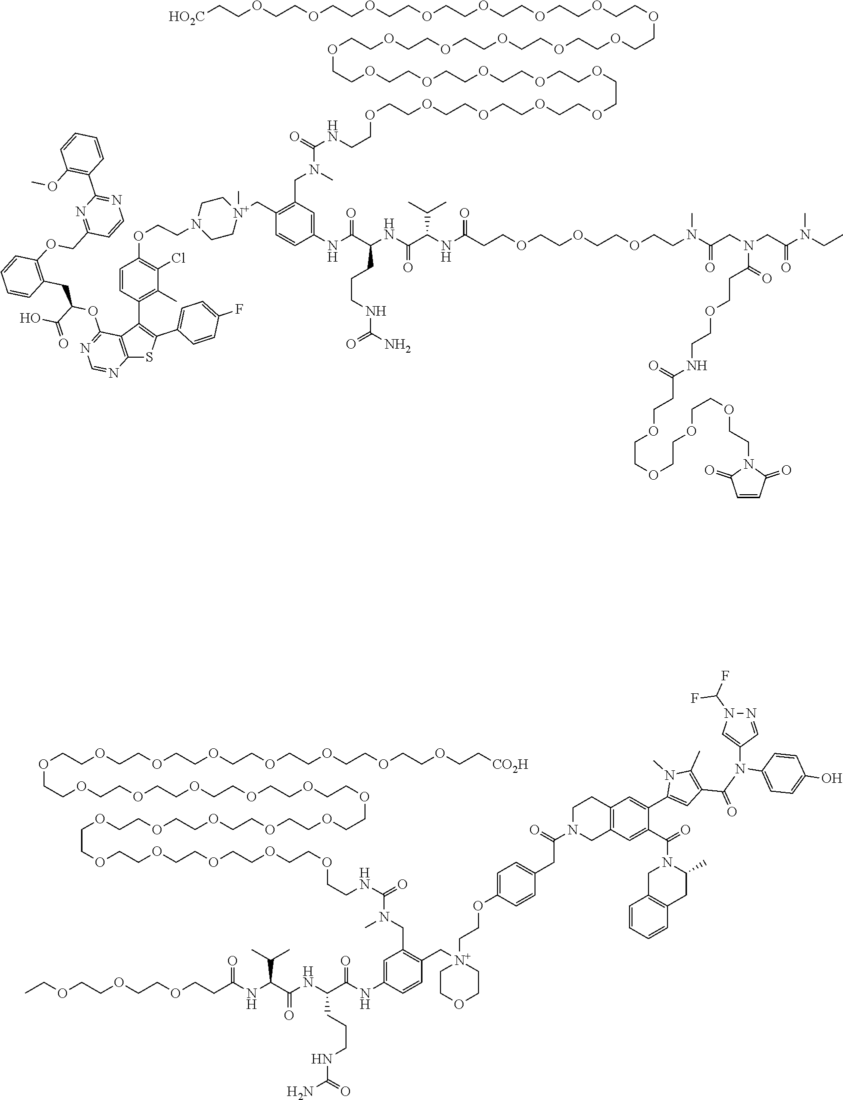

Following GENERAL PROCEDURE #5 using 4(-(S9,3S4S-mn-2-3(-(et 7,10,13,16,19,22,25,28,31,34,37,40,43,46,49,52,55,58,61,64,67,70,73,768-etacosaoxa-2,4-diazadooctacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (147 mg, 25.1 μmol), 2,5-dioxopyrrolidin-1-yl 1-(2,5-dioxo-2,5-dihydro-1H-pyrrol-1-yl)-13,6,9,12-tetraoxapentadecan-15-oate (22.2 mg, 50.3 μmol) and DIEA (65.7 μL, 377 μmol), 4-(4-((6S,9S,43S,46S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((1-(difluoromethyl)-1H-pyrazol-4-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (76 mg, 45% yield) was obtained. HRMS: (M⁺²-H⁺)+=5897.9502, Rt=2.42 min (5 min acidic method).

### Synthesis of 4-(2-(((((9H-fluoren-9-yl)methoxy)carbonyl)(methyl)amino)methyl)-4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

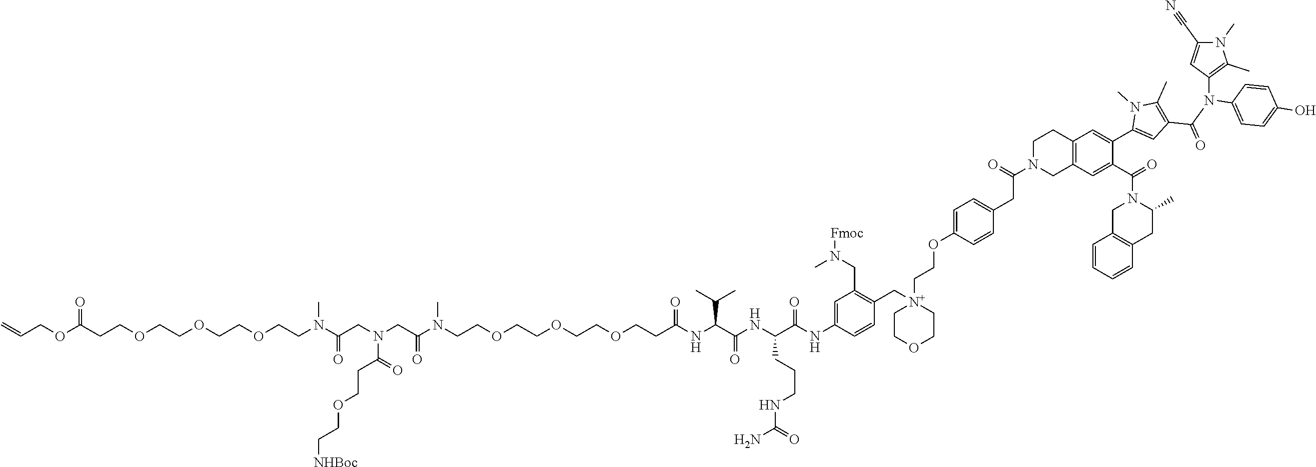

Following GENERAL PROCEDURE #3 using 4-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (176.8 mg, 100.7 μmol) and 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (95.2 mg, 115.8 μmol), 4-(2-(((((9H-fluoren-9-yl)methoxy)carbonyl)(methyl)amino)methyl)-4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (229 mg, 93%) was obtained. HRMS: M⁺=2331.2100, Rt=2.54 min (5 min acidic method).

### Synthesis of 4-(2-(((((9H-fluoren-9-yl)methoxy)carbonyl)(methyl)amino)methyl)-4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

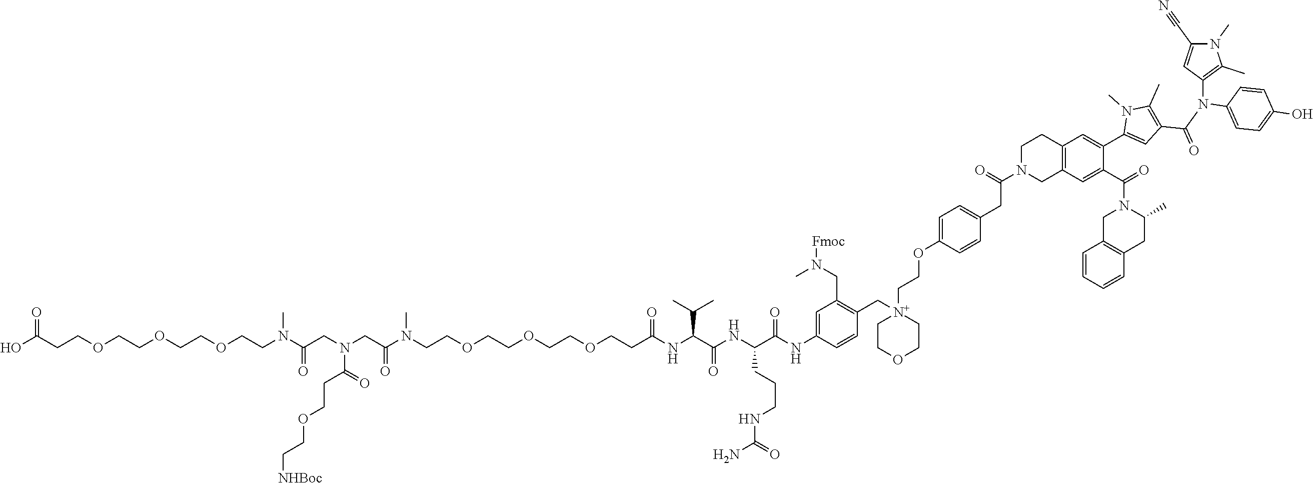

Following GENERAL PROCEDURE #2 using 4-(2-(((((9H-fluoren-9-yl)methoxy)carbonyl)(methyl)amino)methyl)-4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium Trifluoroacete (229 mg, 93.6 μmol), 4-(2-(((((9H-fluoren-9-yl)methoxy)carbonyl)(methyl)amino)methyl)-4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (144 mg, 62% yield) was obtained. HRMS: M⁺=2291.2300, Rt=2.39 min (5 min acidic method).

### Synthesis of 4-(2-(((((9H-fluoren-9-yl)methoxy)carbonyl)(methyl)amino)methyl)-4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

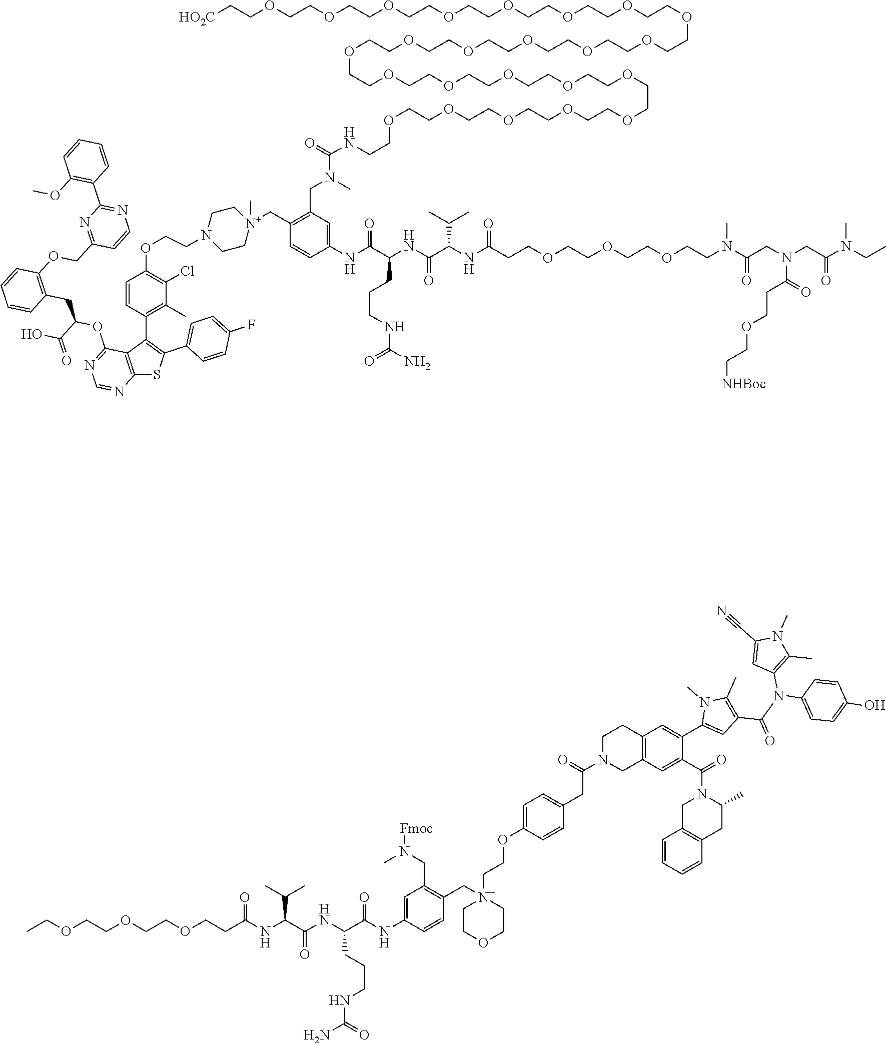

Following GENERAL PROCEDURE #4 using 4-(2-(((((9H-fluoren-9-yl)methoxy)carbonyl)(methyl)amino)methyl)-4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium TFA salt (145.8 mg, 60.6 μmol), TSTU (18.97 mg, 63 μmol), 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium TFA salt (186.8 mg, 69.7 μmol) and DIEA (75 μL, 424 μmol), 4-(2-(((((9H-fluoren-9-yl)methoxy)carbonyl)(methyl)amino)methyl)-4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (155 mg, 54% yield) was obtained. HRMS: (M⁺²-H⁺)+=4723.3198, Rt=2.61 min (5 min acidic method).

### Synthesis of 4-(4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

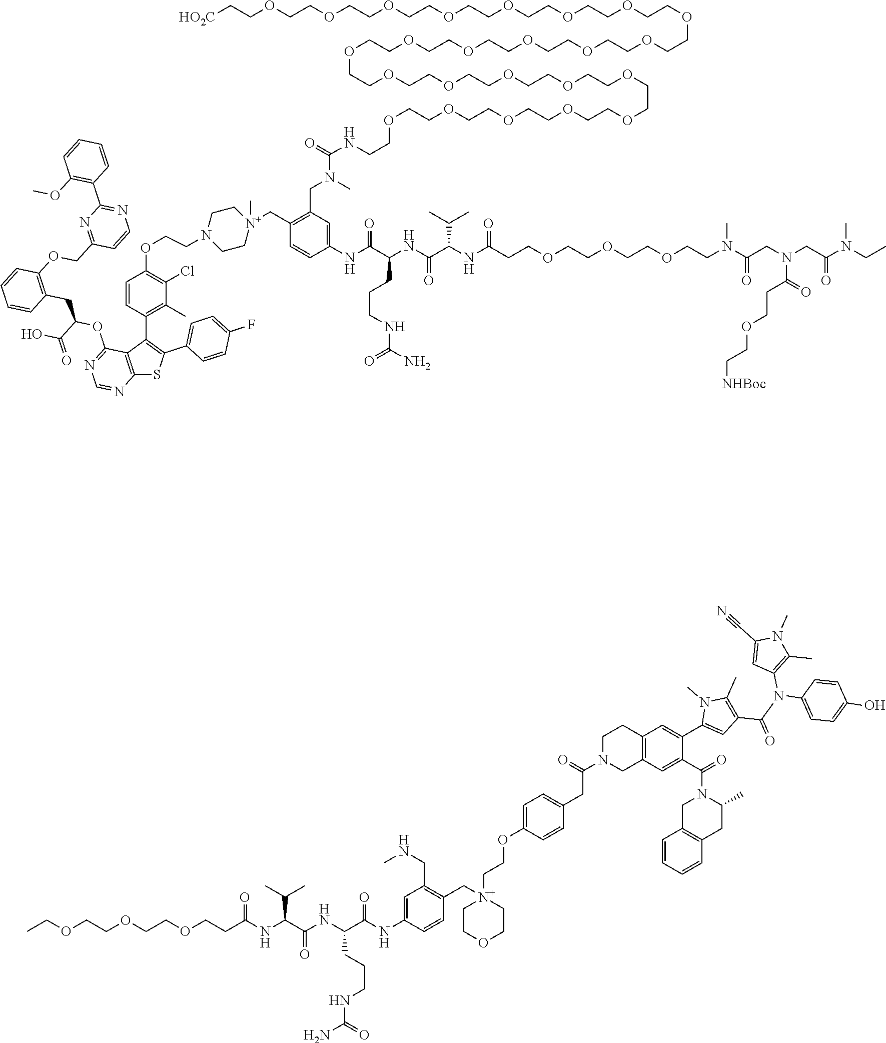

To 4-(2-(((((9H-fluoren-9-yl)methoxy)carbonyl)(methyl)amino)methyl)-4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5 -yl) -2-chloro-3-methylphe noxy) ethyl) -1-methylpiperazin-1l-ium-1-yl) methyl) -3-(78 -carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1 H)-yl)-2-oxoethyl)phenoxy)ethyl)morpholin-4-ium TFA salt (155 mg, 31.3 μmol) in DMF (1.5 mL) was added 2M dimethylamine in MeOH (235 μL, 470 μmol). After standing for 1.5 hours, the solution was diluted with DMSO (3 mL) and purified by C18 RP-HPLC yielding 4-(4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (152 mg, 63%). HRMS: (M⁴2H⁺)+=4501.2402, Rt=2.40 min (5 min acidic method).

### Synthesis of 4-(4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

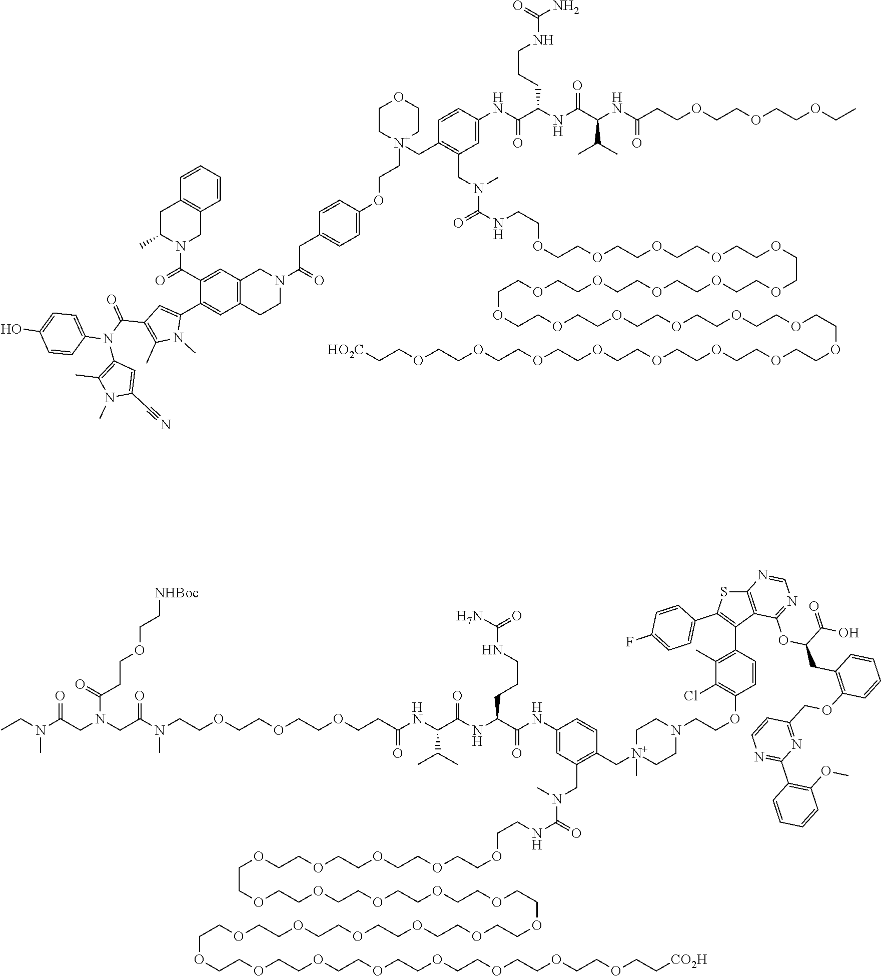

Following GENERAL PROCEDURE #1 using bis(4-nitrophenyl) carbonate (7.3 mg, 24 μmol, 0.95 equiv), DIEA (13 μL, 76 μmol, 3 equiv.), 1-amino-3,6,9,12,15,18,21,24,27,30,33,36,39,42,45,48,51,54,57,60,63,66,69,72-tetracosaoxapentaheptacontan-75-oic acid (29 mg, 25 μmol, 1.0 equiv), and then 4-(4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium TFA salt (92 mg, 19 μmol, 0.75 equiv) and DIEA (27 μL, 150 μmol, 6 equiv), 4-(4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (101 mg, 67% yield) was obtained. HRMS: (M⁺²-H⁺)+=5672.8999, Rt=2.52 min (5 min acidic method).

### Synthesis of 4-(4-((6S,9S,43S,46S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (P1-L12-P4)

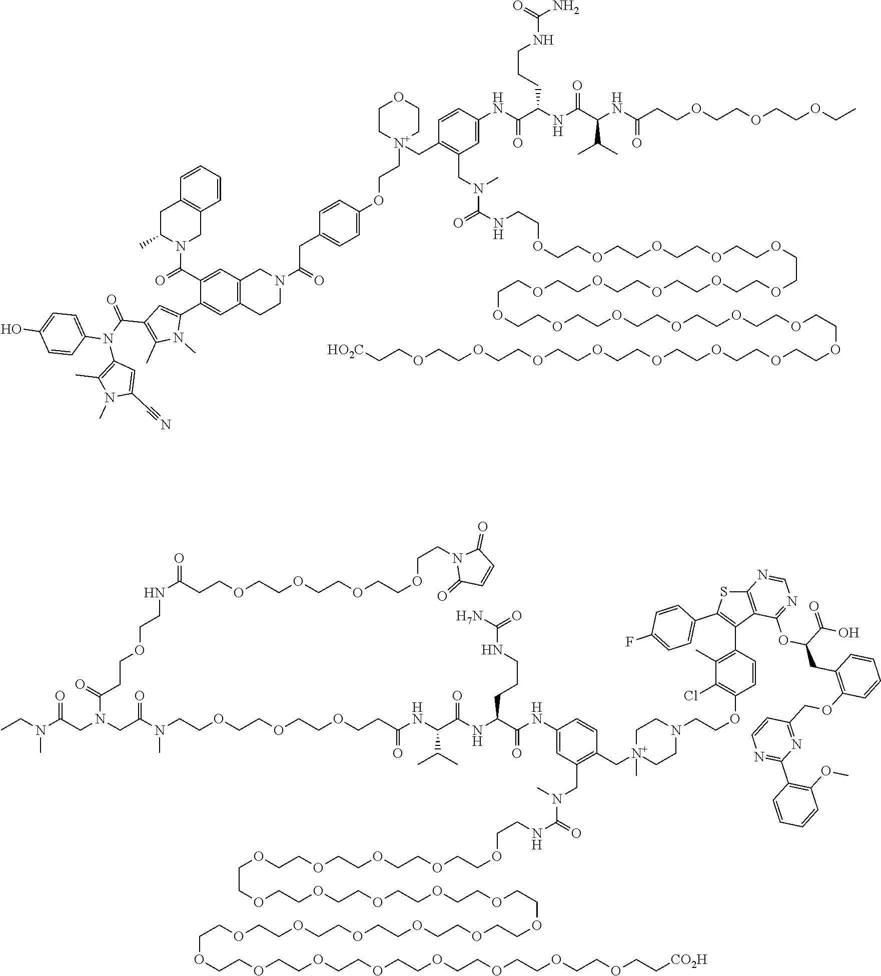

Following GENERAL PROCEDURE #5 and using 4-(4-((6S,9S,43S,46S)-1-amino-26-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (135.7 mg, 21.1 μmol, 1.0 equiv), 2,5-dioxopyrrolidin-1-yl 1-(2,5-dioxo-2,5-dihydro-1H-pyrrol-l-yl)-3,6,9,12-tetraoxapentadecan-15-oate (16.5 mg, 37.3 μmol, 1.75 equiv.) and DIEA (100 μL, 575 μmol, 27 equiv,), 4-(4-((6S,9S,43S,46S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(1-(2,5-dioxo-2,5-dihydro-1H-pyrrol-l-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium bistrifluoroacetate (102 mg, 78% yield) was obtained. HRMS: (M⁺²-H⁺)+=5900.5600, Rt=2.45 min (5 min acidic method).

### Synthesis of 3,3′-((((1r,1′r,3s,3's,5R,5′R,7S,7'S)-((((((((((2S,5S,39S,42S)-22-(3-(2-((tert- butoxycarbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4,1-phenylene))bis(methylene))bis(oxy))bis(carbonyl))bis((3-hydroxypropyl)azanediyl))bis(ethane-2,1-diyl))bis(oxy))bis(5,7-dimethyladamantane-3,1-diyl))bis(methylene))bis(5-methyl-1H-pyrazole-1,4-diyl))bis(6-(3-(benzo[d]thiazol-2-ylamino)-4-methyl-6,7-dihydropyrido[2,3-c]pyridazin-8(5H)-yl)picolinic acid)

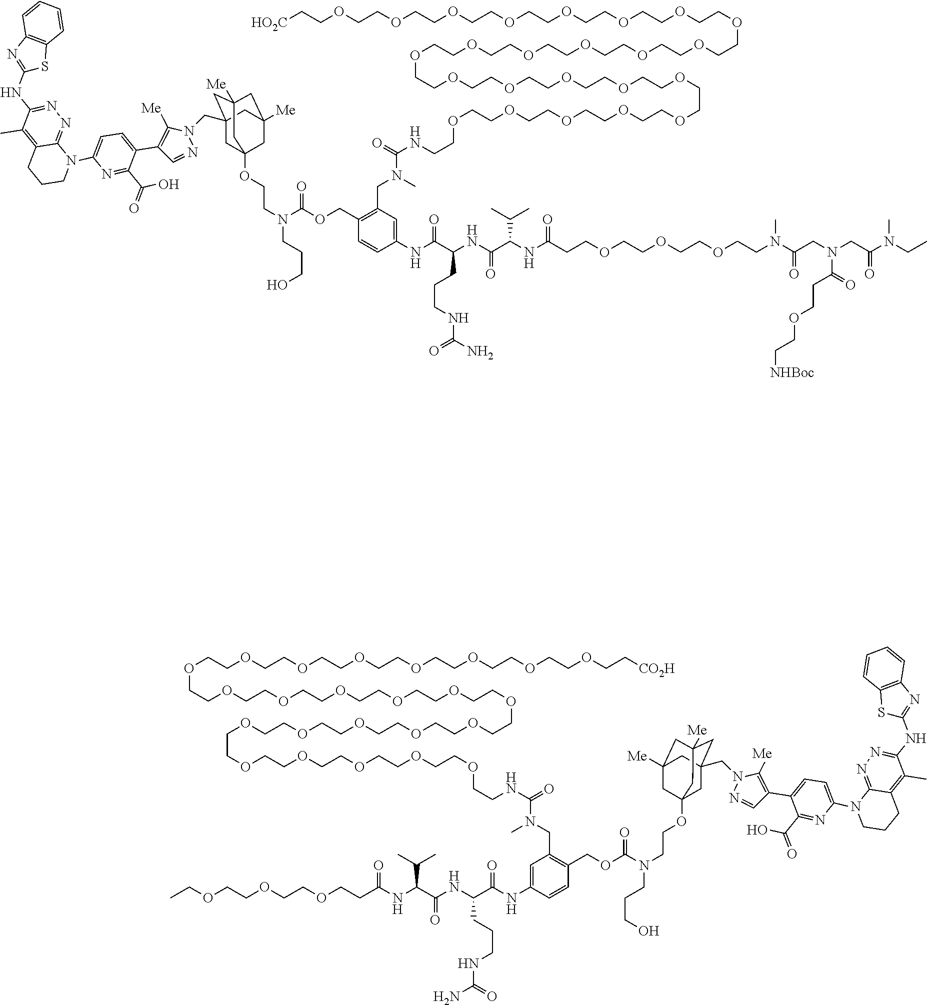

To a solution of 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (110.4 mg, 49 μmol) and bis(2,5-dioxopyrrolidin-1-yl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (20.4 mg, 20.9 μmol) in NMP (0.5 mL) was added DIEA (18.2 μL, 104 μmol). After standing for 7 hours, the solution was diluted with DMSO, purified by RP-HPLC with 0.05% formic acid modifier and after lyophilization 3,3′-((((1r,1′r,3s,3's,5R,5′R,7S,7'S)-((((((((((2S,5S,39S,42S)-22-(3-(2-((tert-butoxycarbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4,1-phenylene))bis(methylene))bis(oxy))bis(carbonyl))bis((3-hydroxypropyl)azanediyl))bis(ethane-2,1-diyl))bis(oxy))bis(5,7-dimethyladamantane-3,1-diyl))bis(methylene))bis(5-methyl-1H-pyrazole-1,4-diyl))bis(6-(3-(benzo[d]thiazol-2-ylamino)-4-methyl-6,7-dihydropyrido[2,3-c]pyridazin-8(5H)-yl)picolinic acid) (96 mg, 83% yield) was obtained. MH⁺=5569.9702, Rt=2.37 min (5 min acidic method).

### Synthesis of 3,3′-((((1r,1′r,3s,3's,5R,5′R,7S,7'S)-((((((((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4,1-phenylene))bis(methylene))bis(oxy))bis(carbonyl))bis((3-hydroxypropyl)azanediyl))bis(ethane-2,1-diyl))bis(oxy))bis(5,7-dimethyladamantane-3,1-diyl))bis(methylene))bis(5-methyl-1H-pyrazole-1,4-diyl))bis(6-(3-(benzo[d]thiazol-2-ylamino)-4-methyl-6,7-dihydropyrido[2 3-c]pyridazin-8(5H)- yl)picolinic acid) (P5-L12-P5)

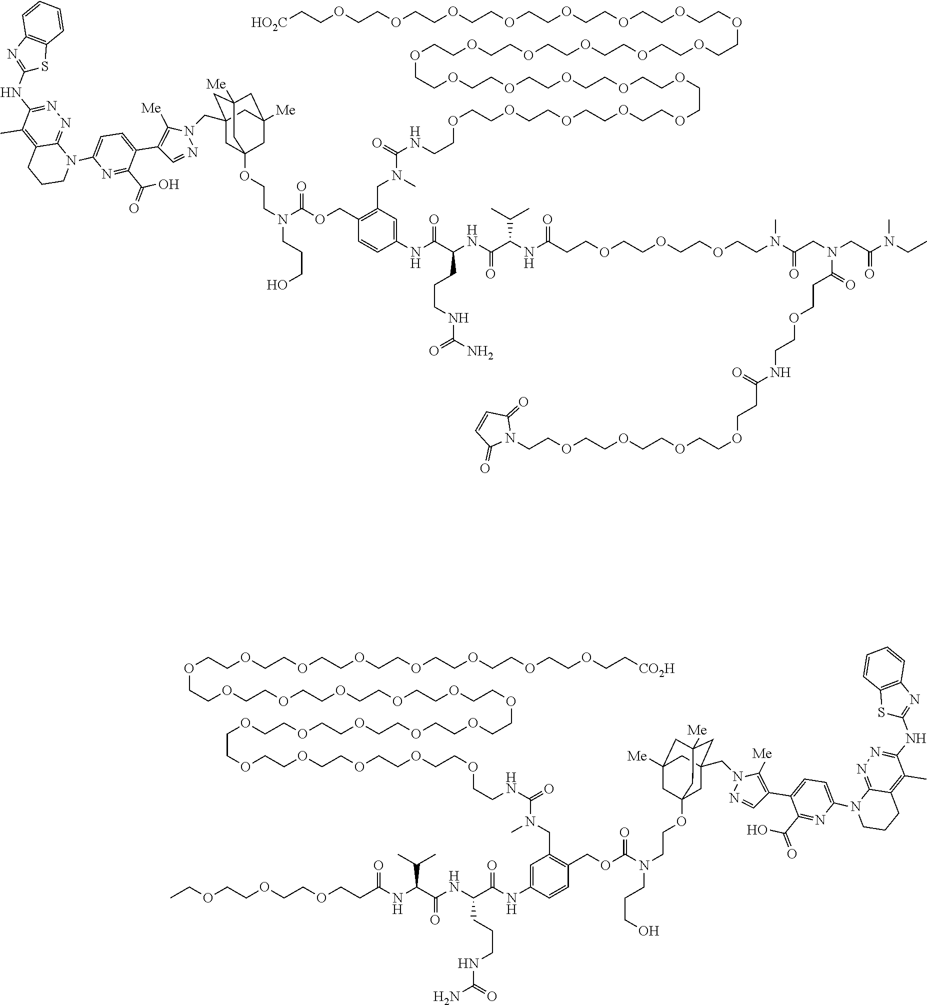

Following the first part of GENERAL PROCEDURE #5 and using 3,3′-((((1r,1′r,3s,3's,5R,5′R,7S,7'S)-((((((((((2S,5S,39S,42S)-22-(3-(2-((tert-butoxycarbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4,1-phenylene))bis(methylene))bis(oxy))bis(carbonyl))bis((3-hydroxypropyl)azanediyl))bis(ethane-2,1-diyl))bis(oxy))bis(5,7-dimethyladamantane-3,1-diyl))bis(methylene))bis(5-methyl-1H-pyrazole-1,4-diyl))bis(6-(3-(benzo[d]thiazol-2-ylamino)-4-methyl-6,7-dihydropyrido[2,3-c]pyridazin-8(5H)-yl)picolinic acid) (153 mg, 27.5 μmol), after removal of volatiles in vacuo the residue was dissolved in DMF (2 mL) and 2M Me₂NH in MeOH (275 μL, 550 μmol) was added to clip the trifluoroacetyl ester that had formed. After standing for 2 hours, the solution was diluted with DMSO and after purification by RP-HPLC with 0.05% formic acid modifier the deprotected amine intermediate was isolated. Following the second part of GENERAL PROCEDURE #5and using isolated amine (57 mg, 10.4 μmol), 2,5-dioxopyrrolidin-1-yl 1-(2,5-dioxo-2,5-dihydro-1H-pyrrol-1-yl)-3,6,9,12-tetraoxapentadecan-15-oate (6.9 mg, 15.6 μmol) and DIEA (22 μL, 125 μmol), 3,3′-((((1r,1′r,3s,3's,5R,5′R,7S,7'S)-((((((((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4,1-phenylene))bis(methylene))bis(oxy))bis(carbonyl))bis((3-hydroxypropyl)azanediyl))bis(ethane-2,1-diyl))bis(oxy))bis(5,7-dimethyladamantane-3,1-diyl))bis(methylene))bis(5-methyl-1H-pyrazole-1,4-diyl))bis(6-(3-(benzo[d]thiazol-2-ylamino)-4-methyl-6,7-dihydropyrido[2,3-c]pyridazin-8(5H)-yl)picolinic acid) (48.5 mg, 30% yield) was obtained after RP-HPLC with 0.05% formic acid modifier and lyophilization. HRMS: MH⁺=5797.1401, Rt=2.34 min (5 min acidic method).

### Synthesis of N-(4-((6S,31S,34S)-6-(2-carboxyethyl)-31-isopropyl-2,2-dimethyl-4,7,29,32-tetraoxo-34-(3-ureidopropyl)-3,11,14,17,20,23,26-heptaoxa-5,8,30,33-tetraazapentatriacontan-35-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

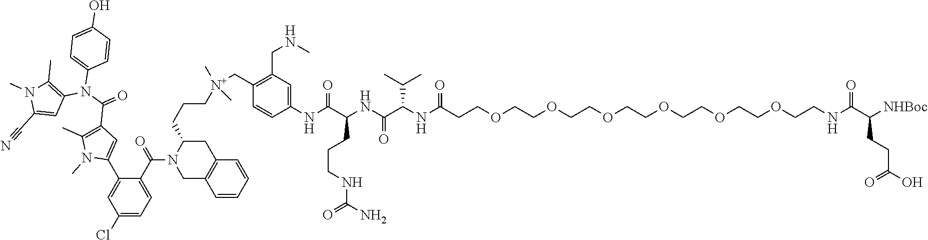 [SMILES: CNCc1cc(NC(=O)[C@H](CCCNC(N)=O)NC(=O)[C@@H](NC(=O)CCOCCOCCOCCOCCOCCOCCNC(=O)[C@H](CCC(=O)O)NC(=O)OC(C)(C)C)C(C)C)ccc1C[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C]

To a solution of N-(2-(((((9H-fluoren-9-yl)methoxy)carbonyl)(methyl)amino)methyl)-4-((S)-2-((S)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (300 mg, 208 μmol) and (S)-6-(3-methoxy-3-oxopropyl)-2,2-dimethyl-4,7-dioxo-3,11,14,17,20,23,26-heptaoxa-5,8-diazanonacosan-29-oic acid (136 mg, 228 μmol) in DMF (2 mL) was added HATU (87 mg, 228 μmol) and DIEA (145 μL, 850 μmol). After stirring for one hour, LiOH (149 mg, 6230 μmol) was added. After stirring for one hour, acetic acid (480 μL, 8300 μmol) was added to neutralize, the solution was diluted with DMSO and purified by RP-HPLC. Upon lyophilization, N-(4-((6S,31S,34S)-6-(2-carboxyethyl)-31-isopropyl-2,2-dimethyl-4,7,29,32-tetraoxo-34-(3-ureidopropyl)-3,11,14,17,20,23,26-heptaoxa-5,8,30,33-tetraazapentatriacontan-35-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (213 mg, 61% yield) was obtained. HRMS: M⁺=1671.8700, Rt=1.87 min (5 min acidic method).

### Synthesis of N-(4-((6S,31S,34S)-6-(2-carboxyethyl)-31-isopropyl-2,2-dimethyl-4,7,29,32-tetraoxo-34-(3-ureidopropyl)-3,11,14,17,20,23,26-heptaoxa-5,8,30,33-tetraazapentatriacontan-35-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

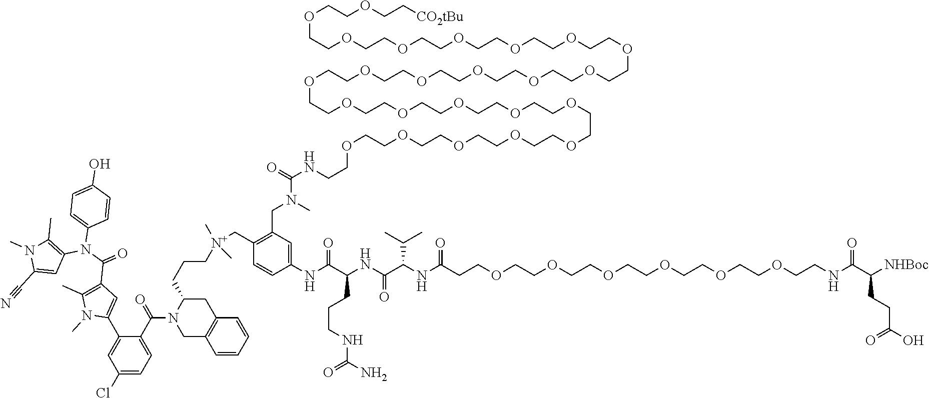 [SMILES: Cc1c(N(C(=O)c2cc(-c3cc(Cl)ccc3C(=O)N3Cc4ccccc4C[C@H]3CCC[N+](C)(C)Cc3ccc(NC(=O)[C@H](CCCNC(N)=O)NC(=O)[C@@H](NC(=O)CCOCCOCCOCCOCCOCCOCCNC(=O)[C@H](CCC(=O)O)NC(=O)OC(C)(C)C)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)n(C)c2C)c2ccc(O)cc2)cc(C#N)n1C]

Following GENERAL PROCEDURE #1 using bis(4-nitrophenyl) carbonate (21.2 mg, 70 μmol), DIEA (29 μL, 166 μmol), tert-butyl 1-amino-3,6,9,12,15,18,21,24,27,30,33,36,39,42,45,48,51,54,57,60,63,66,69,72-tetracosaoxapentaheptacontan-75-oate (85 mg, 70 μmol) and then N-(4-((6S,3IS,34S)-6-(2-carboxyethyl)-31-isopropyl-2,2-dimethyl-4,7,29,32-tetraoxo-34-(3-ureidopropyl)-3,11,14,17,20,23,26-heptaoxa-5,8,30,33-tetraazapentatriacontan-35-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (111 mg, 66 μmol) and more DIEA (145 μL, 830 μmol), N-(4-((6S,31S,34S)-6-(2-carboxyethyl)-31-isopropyl-2,2-dimethyl-4,7,29,32-tetraoxo-34-(3-ureidopropyl)-3,11,14,17,20,23,26-heptaoxa-5,8,30,33-tetraazapentatriacontan-35-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (141 mg, 73%) was obtained. HRMS: M⁺=2899.5001, Rt=2.39 min (5 min acidic method).

### Synthesis of 1-(4-((6S,9S,34S,39S,42S)-1-amino-34-((tert-butoxycarbonyl)amino)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,39-diisopropyl-1,8,11,33,37,40-hexaoxo-42-(3-ureidopropyl)-14,17,20,23,26,29-hexaoxa-2,7,10,32,38,41-hexaazatritetracontan-43-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

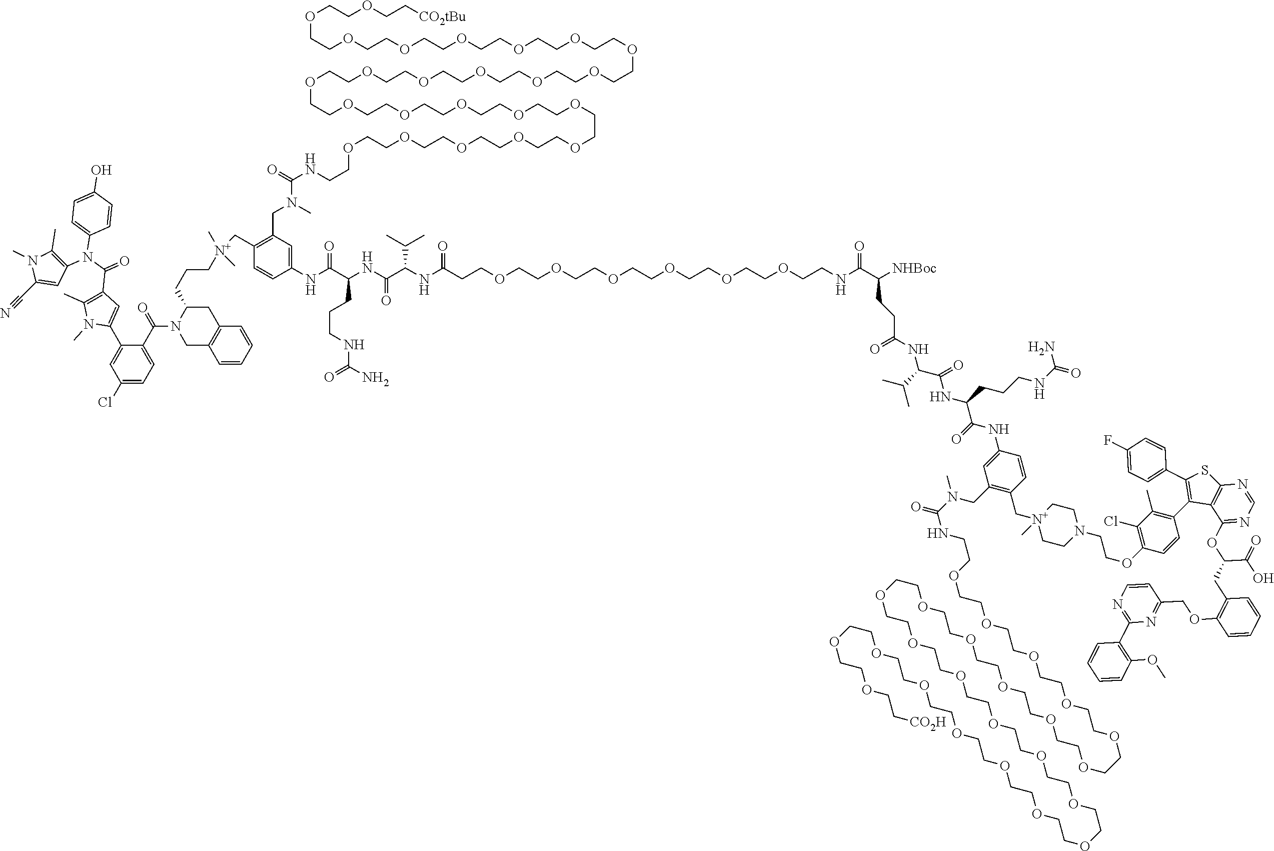

Following GENERAL PROCEDURE #4 using of N-(4-((6S,31S,34S)-6-(2-carboxyethyl)-31-isopropyl-2,2-dimethyl-4,7,29,32-tetraoxo-34-(3-ureidopropyl)-3,11,14,17,20,23,26-heptaoxa-5,8,30,33-tetraazapentatriacontan-35-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (141 mg, 48 μmol), TSTU (14.6 mg, 48 μmol), DIEA (42 μL, 242 μmol) and then 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium TFA salt (137 mg, 56 μmol) and DIEA (42 μL, 242 μmol), 1-(4-((6S,9S,34S,39S,42S)-1-amino-34-((tert-butoxycarbonyl)amino)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,39-diisopropyl-1,8,11,33,37,40-hexaoxo-42-(3-ureidopropyl)-14,17,20,23,26,29-hexaoxa-2,7,10,32,38,41-hexaazatritetracontan-43-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (119 mg, 45% yield) was obtained. HRMS: (M⁺²-H⁺)+=5531.7100, Rt=2.62 min (5 min acidic method).

### Synthesis of 1-(4-((6S,9S,34S,39S,42S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-34-(1-(2,5-dioxo-2,5-dihydro-1H-pyrrol-1-yl)-3,6,9,12,15,18,21,24-octaoxaheptacosan-27-amido)-9,39-diisopropyl-1,8,11,33,37,40-hexaoxo-42-(3-ureidopropyl)-14,17,20,23,26,29-hexaoxa-2,7,10,32,38,41-hexaazatritetracontan-43-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P2-L14-P1)

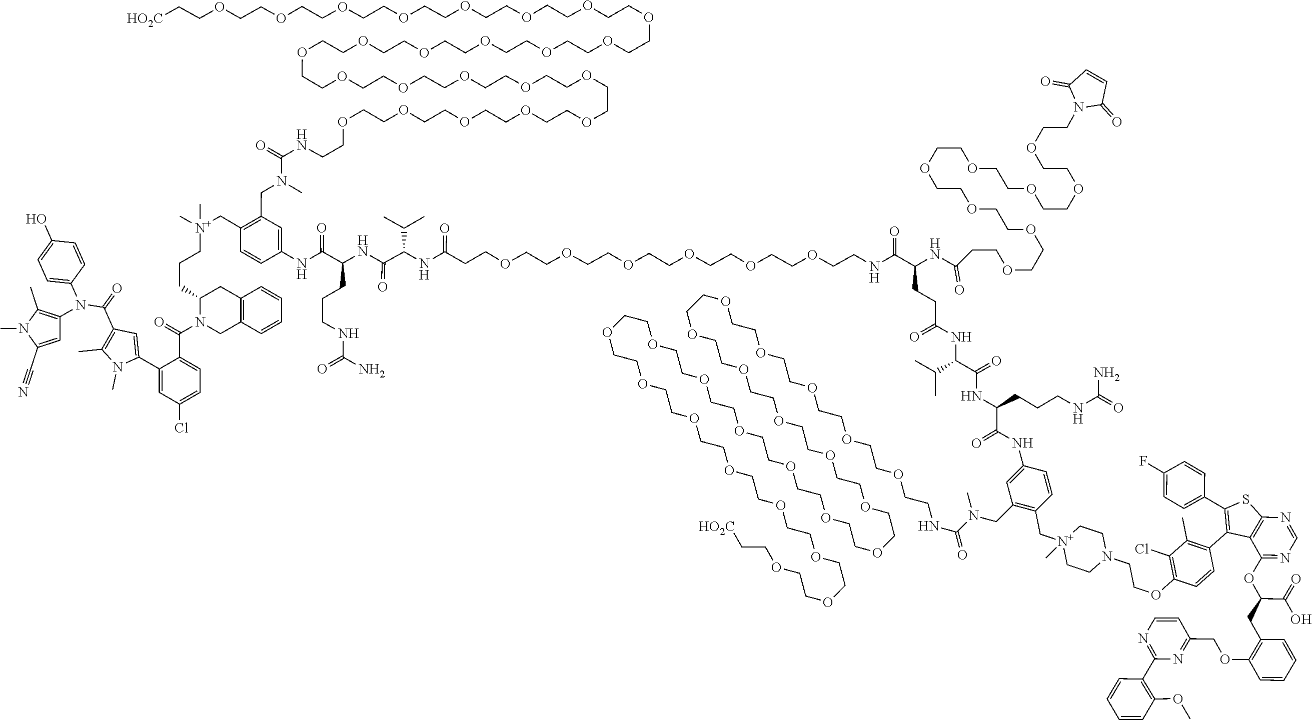

Following the GENERAL PROCEDURE #5 using 1-(4-((6S,9S,34S,39S,42S)-1-amino-34-((tert-butoxycarbonyl)amino)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,39-diisopropyl-1,8,11,33,37,40-hexaoxo-42-(3-ureidopropyl)-14,17,20,23,26,29-hexaoxa-2,7,10,32,38,41-hexaazatritetracontan-43-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (76.4 mg, 14 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (39.4 mg, 64 μmol) and DIEA (45 μL, 55 μmol), 1-(4-((6S,9S,34S,39S,42S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-34-(1-(2,5-dioxo-2,5-dihydro-1H-pyrrol-l-yl)-3,6,9,12,15,18,21,24-octaoxaheptacosan-27-amido)-9,39-diisopropyl-1,8,11,33,37,40-hexaoxo-42-(3-ureidopropyl)-14,17,20,23,26,29-hexaoxa-2,7,10,32,38,41-hexaazatritetracontan-43-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (61.8 mg, 74% yield) was obtained. HRMS: (M⁺²-H⁺)+=5678.8599, Rt=2.49 min (5 min acidic method).

### Synthesis of 1-(4-((S)-2-((S)-2-(2-aminoacetamido)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

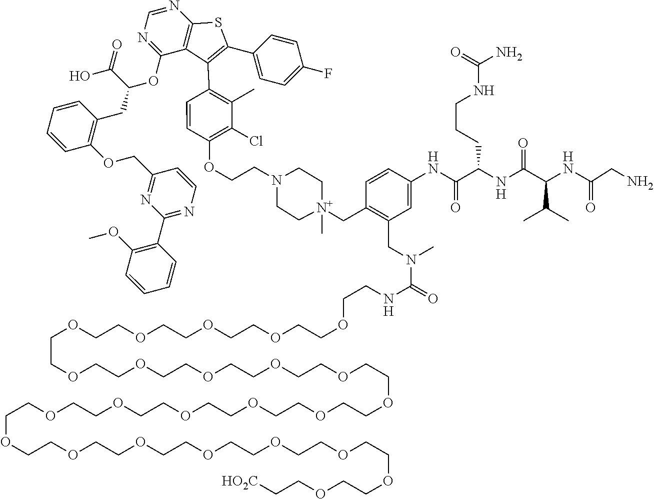 [SMILES: COc1ccccc1-c1nccc(COc2ccccc2C[C@@H](Oc2ncnc3sc(-c4ccc(F)cc4)c(-c4ccc(OCCN5CC[N+](C)(Cc6ccc(NC(=O)[C@H](CCCNC(N)=O)NC(=O)[C@@H](NC(=O)CN)C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC5)c(Cl)c4C)c23)C(=O)O)n1]

To 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (526 mg, 196 μmol) and 2,5-dioxopyrrolidin-1-yl (tert-butoxycarbonyl)glycine (107 mg, 392 μmol) in DMF (3 mL) was added DIEA (205 μL, 1177 μmol). After stirring for 1 hour, the solution was purified by ISCO RP-HPLC. Upon lyophilization the Boc group was removed by treating with 25% TFA/CH2Cl with 0.1% Triethylsilane for 1 hour at which time the volatiles were removed in vacuo, the residue was dissolved in DMSO and purified by ISCO RP-HPLC. Upon lyophilization, 1-(4-((S)-2-((S)-2-(2-aminoacetamido)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (330 mg, 64% yield) was obtained. HRMS: M⁺=2508.2200, Rt=2.20 min (5 min acidic method).

### Synthesis of 1-(4-((6S,9S,34S,42S,45S)-1-amino-34-((tert-butoxycarbonyl)amino)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-1,8,11,33,37,40,43-heptaoxo-45-(3-ureidopropyl)-14,17,20,23,26,29-hexaoxa-2,7,10,32,38,41,44-heptaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

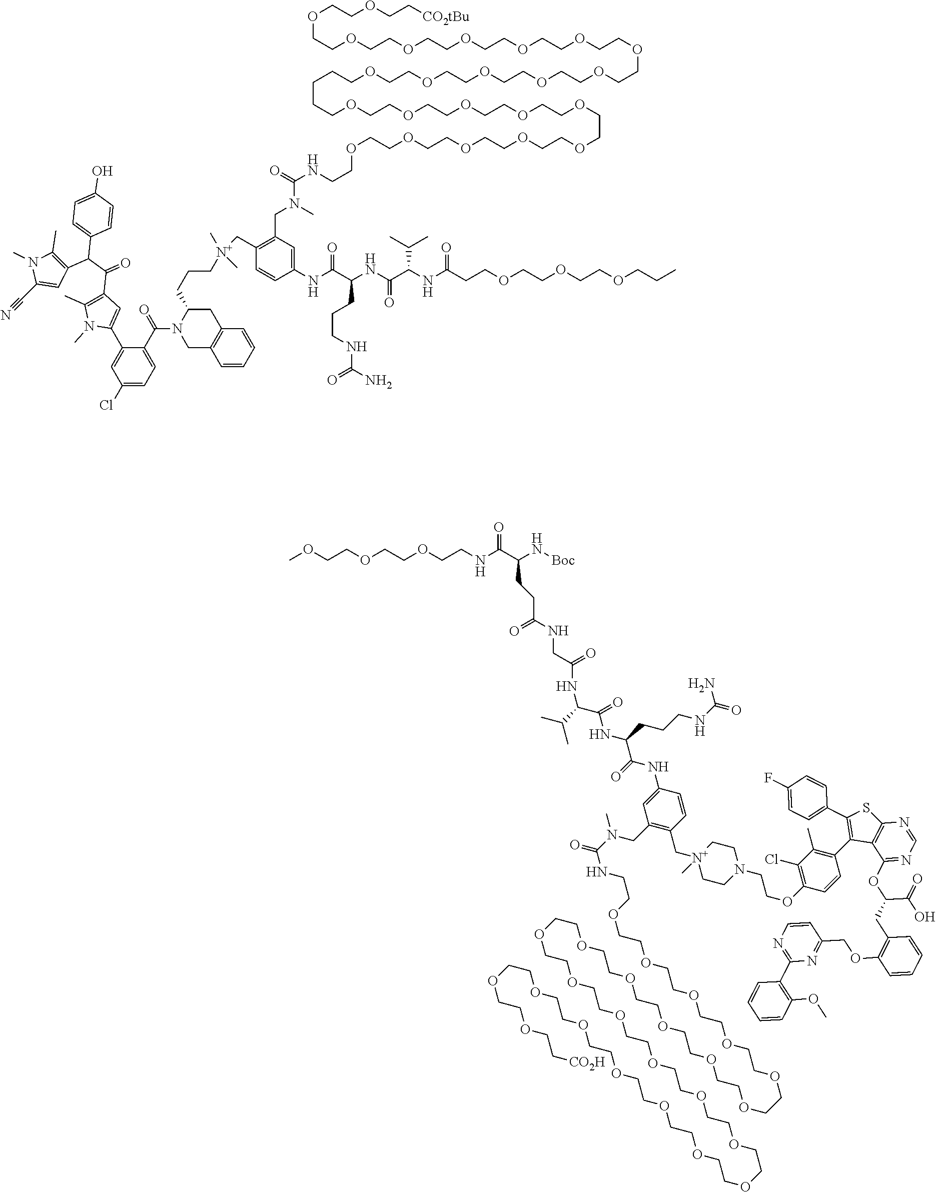

Following GENERAL PROCEDURE #4 using N-(4-((6S,31S,34S)-6-(2-carboxyethyl)-31-isopropyl-2,2-dimethyl-4,7,29,32-tetraoxo-34-(3-ureidopropyl)-3,11,14,17,20,23,26-heptaoxa-5,8,30,33-tetraazapentatriacontan-35-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (189 mg, 65 μmol), TSTU (19.6 mg, 65 μmol), DIEA (91 μL, 521 μmol) and then 1-(4-((S)-2-((S)-2-(2-aminoacetamido)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (188 mg, 75 μmol) and DIEA (91 μL, 521 μmol), 1-(4-((6S,9S,34S,42S,45S)-1-amino-34-((tert-butoxycarbonyl)amino)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-1,8,11,33,37,40,43-heptaoxo-45-(3-ureidopropyl)-14,17,20,23,26,29-hexaoxa-2,7,10,32,38,41,44-heptaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (141 mg, 40% yield) was obtained. HRMS: (M⁺2-H⁺)+=5388.7700, Rt=2.61 min (5 min acidic method).

### Synthesis of 1-(4-((6S,9S,34S,42S,45S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-34-(1-(2,5-dioxo-2,5-dihydro-1H-pyrrol-1-yl)-3,6,9,12,15,18,21,24-octaoxaheptacosan-27-amido)-9,42-diisopropyl-1,8,11,33,37,40,43-heptaoxo-45-(3-ureidopropyl)-14,17,20,23,26,29-hexaoxa-2,7,10,32,38,41,44-heptaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P2-L13-P1)

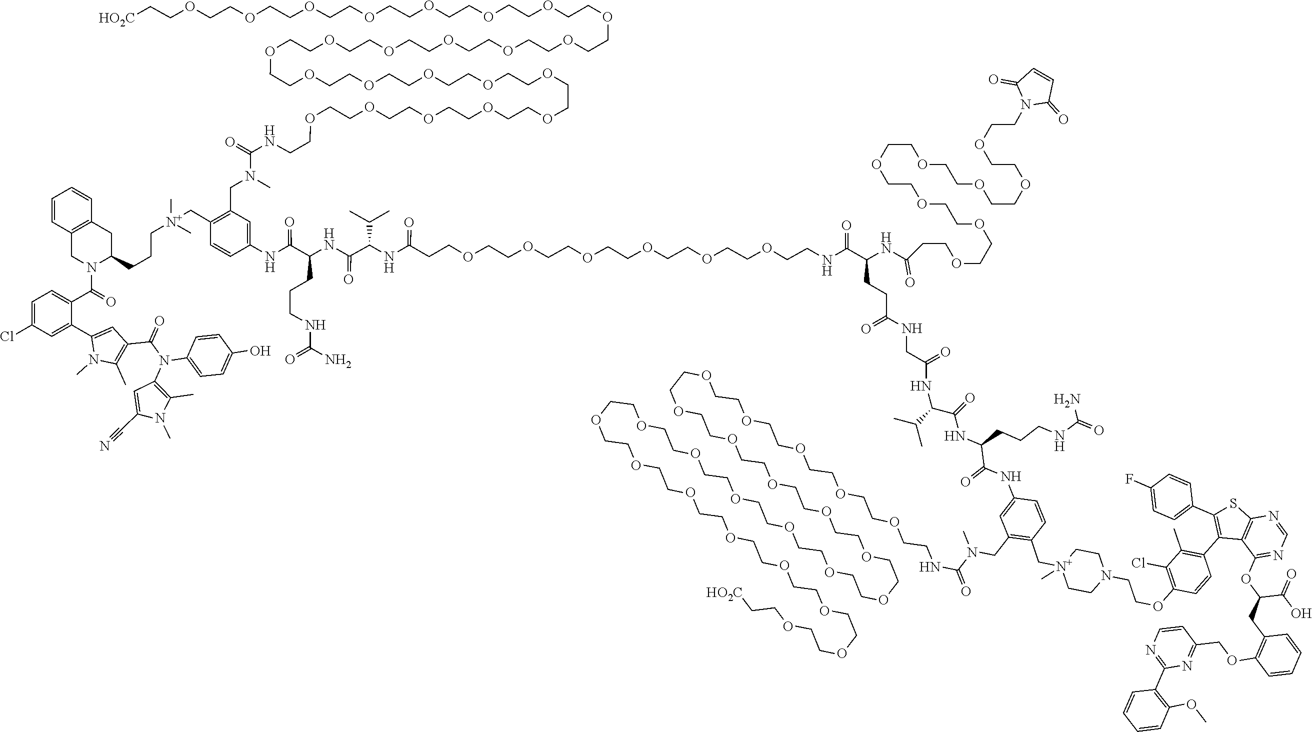

Following the GENERAL PROCEDURE #5 using 1-(4-((6S,9S,34S,42S,45S)-1-amino-34-((tert-butoxycarbonyl)amino)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-1,8,11,33,37,40,43-heptaoxo-45-(3-ureidopropyl)-14,17,20,23,26,29-hexaoxa-2,7,10,32,38,41,44-heptaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (228 mg, 42 μmol), 2,5-dioxopyrrolidin-1-yl 1-(2,5-dioxo-2,5-dihydro-1H-pyrrol-1-yl)-13,6,9,12,15,18,21,24-octaoxaheptacosan-27-oate (130 mg, 210 μmol) and DIEA (147 μL, 840 μmol), 1-(4-((6S,9S,34S,42S,45S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-34-(1-(2,5-dioxo-2,5-dihydro-1H-pyrrol-1-yl)-3,6,9,12,15,18,21,24-octaoxaheptacosan-27-amido)-9,42-diisopropyl-1,8,11,33,37,40,43-heptaoxo-45-(3-ureidopropyl)-14,17,20,23,26,29-hexaoxa-2,7,10,32,38,41,44-heptaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (86 mg, 34% yield) was obtained. HRMS: (M⁺²-H⁺)+=5735.8799, Rt=2.49 min (5 min acidic method).

### Synthesis of N-(2-(((((9H-fluoren-9-yl)methoxy)carbonyl)(methyl)amino)methyl)-4-((2S,5S)-5-isopropyl-21,21-dimethyl-4,7,19-trioxo-2-(3-ureidopropyl)-10,13,16,20-tetraoxa-3,6-diazadocosanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

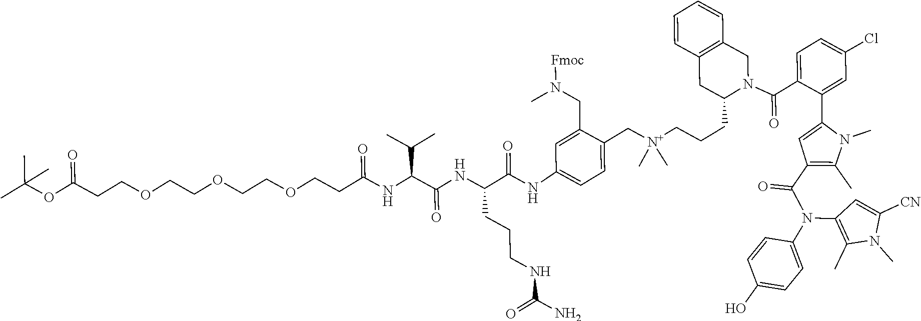

To a solution of 2,2-dimethyl-4-oxo-3,7,10,13-tetraoxahexadecan-16-oic acid (47.2 mg, 154 μmol) and HATU (51.2 mg, 135 μmol) in DMF (2 mL) was added DIEA (112 p L, 641 μmol). After stirring for 10 minutes, N-(2-(((((9H-fluoren-9-yl)methoxy)carbonyl)(methyl)amino)methyl)-4-((S)-2-((S)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (200 mg, 128 μmol) was added. After stirring an additional 30 minutes, the solution was diluted with DMSO and purified by ISCO RP-HPLC. Upon lyophilization, N-(2-(((((9H-fluoren-9-yl)methoxy)carbonyl)(methyl)amino)methyl)-4-((2S,5S)-5-isopropyl-21,21-dimethyl-4,7,19-trioxo-2-(3-ureidopropyl)-10,13,16,20-tetraoxa-3,6-diazadocosanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (153 mg, 69% yield) was obtained. HRMS: M⁺=1617.8101, Rt=2.68 min (5 min acidic method).

### Synthesis of N-(4-((14S,17S)-1-carboxy-14-isopropyl-12,15-dioxo-17-(3-ureidopropyl)-3,6,9-trioxa-13,16-diazaoctadecan-18-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

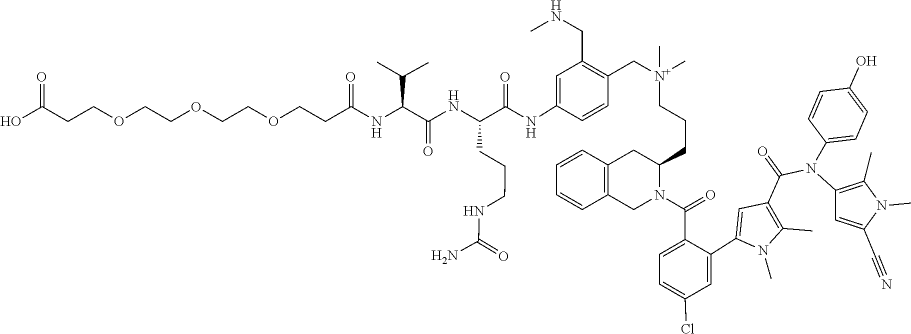 [SMILES: CNCc1cc(NC(=O)[C@H](CCCNC(N)=O)NC(=O)[C@@H](NC(=O)CCOCCOCCOCCC(=O)O)C(C)C)ccc1C[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C]

N-(2-(((((9H-fluoren-9-yl)methoxy)carbonyl)(methyl)amino)methyl)-4-((2S,5S)-5-isopropyl-21,21-dimethyl-4,7,19-trioxo-2-(3-ureidopropyl)-10,13,16,20-tetraoxa-3,6-diazadocosanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (153 mg, 88 μmol) was treated with 25% TFA/CH₂Cl₂ (4 mL) for 1 hour at which time the volatiles were removed in vacuo. The residue was dissolved in DMSO (1 mL) and 2.0 M dimethyl amine in MeOH (1.32 mL, 2650 μmol) was added. After stirring for 1 hour, the solution was directly purified by ISCO RP-HPLC. Upon lyophilization, N-(2-(((((9H-fluoren-9-yl)methoxy)carbonyl)(methyl)amino)methyl)-4-((2S,5S)-5-isopropyl-21,21-dimethyl-4,7,19-trioxo-2-(3-ureidopropyl)-10,13,16,20-tetraoxa-3,6-diazadocosanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (119 mg, 86% yield) was obtained. HRMS: M⁺=1339.6700, Rt=1.74 min (5 min acidic method).

### Synthesis of N-(4-((14S,17S)-1-carboxy-14-isopropyl-12,15-dioxo-17-(3-ureidopropyl)-3,6,9-trioxa-13,16-diazaoctadecan-18-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

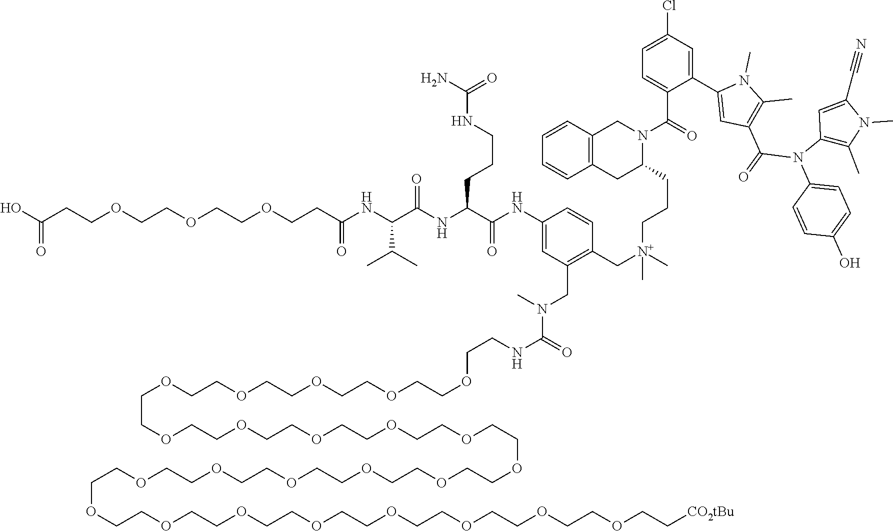 [SMILES: Cc1c(N(C(=O)c2cc(-c3cc(Cl)ccc3C(=O)N3Cc4ccccc4C[C@H]3CCC[N+](C)(C)Cc3ccc(NC(=O)[C@H](CCCNC(N)=O)NC(=O)[C@@H](NC(=O)CCOCCOCCOCCC(=O)O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)n(C)c2C)c2ccc(O)cc2)cc(C#N)n1C]

Following GENERAL PROCEDURE #1 using bis(4-nitrophenyl) carbonate (24.2 mg, 80 μmol), DIEA (199 μL, 1138 μmol), tert-butyl 1-amino-3,6,9,12,15,18,21,24,27,30,33,36,39,42,45,48,51,54,57,60,63,66,69,72-tetracosaoxapentaheptacontan-75-oate (96 mg, 80 μmol) and then N-(4-((14S,17S)-1-carboxy-14-isopropyl-12,15-dioxo-17-(3-ureidopropyl)-3,6,9-trioxa-13,16-diazaoctadecan-18-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (119 mg, 76 μmol) and additional DIEA, N-(4-((14S,17S)-1-carboxy-14-isopropyl-12,15-dioxo-17-(3-ureidopropyl)-3,6,9-trioxa-13,16-diazaoctadecan-18-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (189 mg, 93%) was obtained. HRMS: M⁺=2567.3799, Rt=2.38 min (5 min acidic method).

### Synthesis of N-(4-((2S,5S)-23-(3-(2-((((9H-fluoren-9-yl)methoxy)carbonyl)amino)ethoxy)propanoyl)-35-carboxy-5-isopropyl-20,26-dimethyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

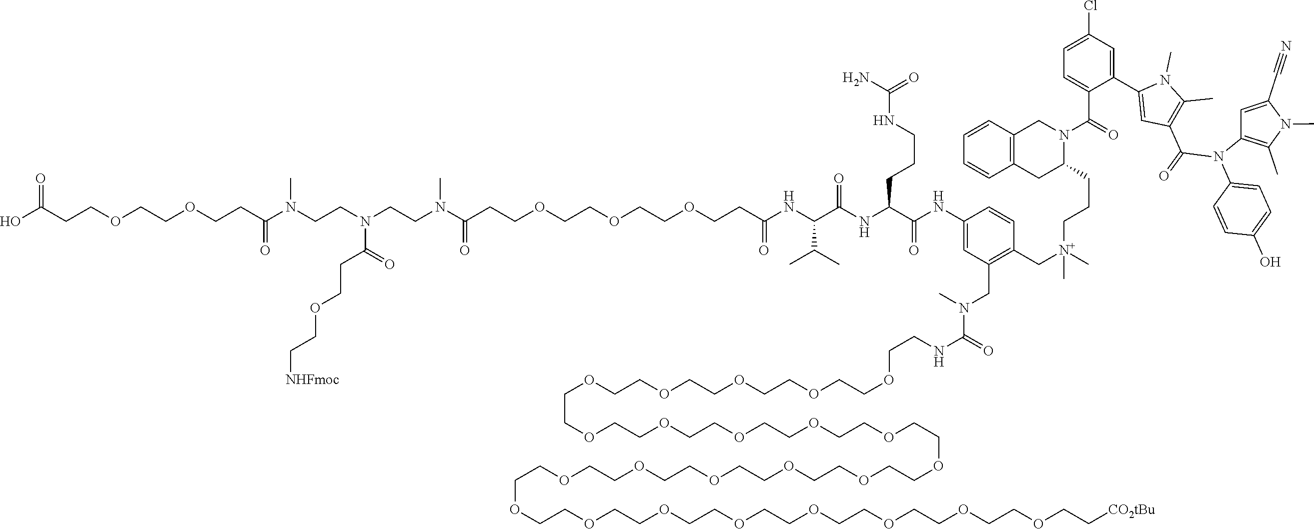

To a solution of N-(4-((14S,17S)-1-carboxy-14-isopropyl-12,15-dioxo-17-(3-ureidopropyl)-3,6,9-trioxa-13,16-diazaoctadecan-18-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (60 mg, 22 μmol) and HATU (6.8 mg, 18 μmol) in DMF was added DIEA (39 μL, 224 μmol). After stirring for 10 minutes, 1-(9H-fluoren-9-yl)-14-methyl-11-(2-(methylamino)ethyl)-3,10,15-trioxo-2,7,18,21-tetraoxa-4,11,14-triazatetracosan-24-oic acid (27.4 mg, 22 μmol) was added. After stirring for 15 minutes, the solution was diluted with DMSO and purified by ISCO RP-HPLC. Upon lyophilization, N-(4-((2S,5S)-23-(3-(2-((((9H-fluoren-9-yl)methoxy)carbonyl)amino)ethoxy)propanoyl)-35-carboxy-5-isopropyl-20,26-dimethyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (34 mg, 45% yield) was obtained. HRMS: M⁺=3205.7200, Rt=2.59 min (5 min acidic method).

### Synthesis of 1-(4-((6S,9S,42S,45S)-1-amino-27-(3-(2-aminoethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-24,30-dimethyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

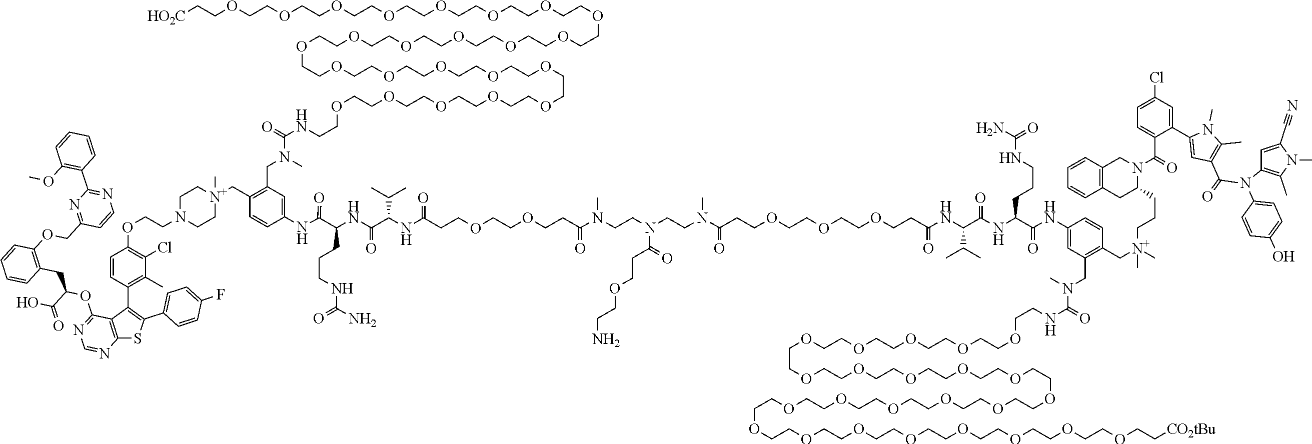

Following GENERAL PROCEDURE #4 using N-(4-((2S,5S)-23-(3-(2-((((9H-fluoren-9-yl)methoxy)carbonyl)amino)ethoxy)propanoyl)-35-carboxy-5-isopropyl-20,26-dimethyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (65.2 mg, 20 μmol), TSTU (5.9 mg, 20 μmol), DIEA (27.4 p L, 157 μmol) and then 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium TFA salt (57.9 mg, 22 μmol) with the modification of once coupling was complete treating with 2.0 M dimethyl amine in MeOH (196 μL, 393 μmol) for 2 hours, 1-(4-((6S,9S,42S,45S)-1-amino-27-(3-(2-aminoethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-24,30-dimethyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (55.3 mg, 49% yield) was obtained. HRMS: (M⁺²- H⁺)⁺=5415.8398, Rt=2.47 min (5 min acidic method).

### Synthesis of 1-(4-((6S,9S,42S,45S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-27-(1-(2,5-dioxo-2,5-dihydro-1H-pyrrol-1-yl)-15-oxo-3,6,9,12,19-pentaoxa-16-azadocosan-22-oyl)-9,42-diisopropyl-24,30-dimethyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L15-P2)

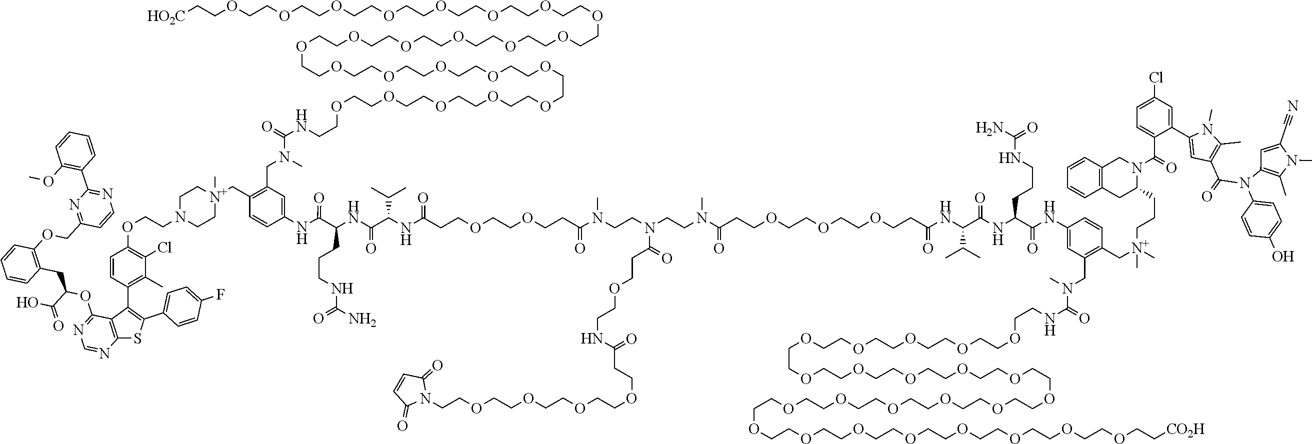

Following the GENERAL PROCEDURE #5 using 1-(4-((6S,9S,42S,45S)-1-amino-27-(3-(2-aminoethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-24,30-dimethyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (55.3 mg, 9.6 μmol), 2,5-dioxopyrrolidin-1-yl 1-(2,5-dioxo-2,5-dihydro-1H-pyrrol-l-yl)-3,6,9,12-tetraoxapentadecan-15-oate (8.5 mg, 19 μmol) and DIEA (37 p L, 212 μmol), 1-(4-((6S,9S,42S,45S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-27-(1-(2,5-dioxo-2,5-dihydro-1H-pyrrol-l-yl)-15-oxo-3,6,9,12,19-pentaoxa-16-azadocosan-22-oyl)-9,42-diisopropyl-24,30-dimethyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (47 mg, 81% yield) was obtained. HRMS: (M⁺²- H⁺)+=5686.8799, Rt=2.46 min (5 min acidic method).

### Synthesis of N-(4-((32S,35S)-15-(3-(2-((tert-butoxycarbonyl)amino)ethoxy)propanoyl)-1-carboxy-32-isopropyl-13,17,30,33-tetraoxo-35-(3-ureidopropyl)-3,6,9,21,24,27-hexaoxa-12,15,18,31,34-pentaazahexatriacontan-36-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

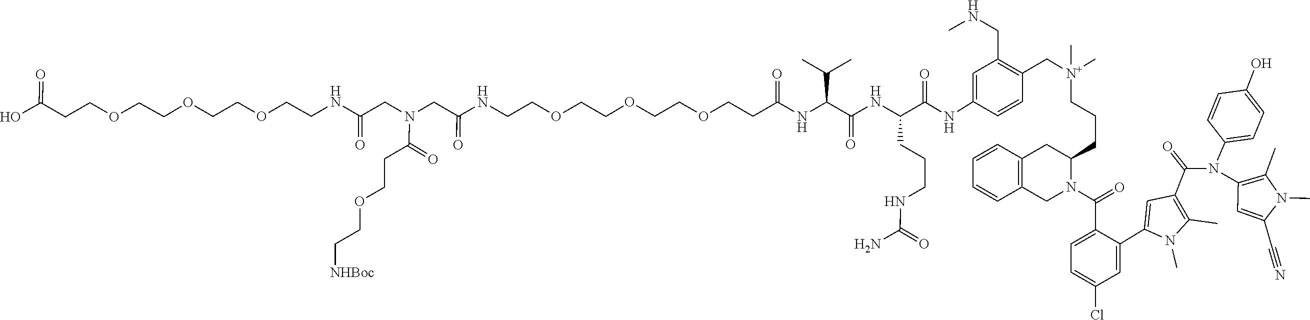 [SMILES: CNCc1cc(NC(=O)[C@H](CCCNC(N)=O)NC(=O)[C@@H](NC(=O)CCOCCOCCOCCNC(=O)CN(CC(=O)NCCOCCOCCOCCC(=O)O)C(=O)CCOCCNC(=O)OC(C)(C)C)C(C)C)ccc1C[N+](C)(C)CCC[C@@H]1Cc2ccccc2CN1C(=O)c1ccc(Cl)cc1-c1cc(C(=O)N(c2ccc(O)cc2)c2cc(C#N)n(C)c2C)c(C)n1C]

To a solution of N-(2-(((((9H-fluoren-9-yl)methoxy)carbonyl)(methyl)amino)methyl)-4-((S)-2-((S)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium (190 mg, 131 μmol), 18-(3-(2-((tert-butoxycarbonyl)amino)ethoxy)propanoyl)-3,16,20-trioxo-2,6,9,12,24,27,30-heptaoxa-15,18,21-triazatritriacontan-33-oic acid (101 mg, 131 μmol) and HATU (50 mg, 131 μmol) in DMF (1 mL) was added DIEA (276 μL, 1578 μmol). After stirring for 15 minutes, MeOH (1.35 mL) and H₂O (0.5 mL) and 1N LiOH (657 μL, 657 μmol) were added. After stirring for 1 hour, additional 1N LiOH (657 μL, 657 μmol) was added. After stirring an additional 10 minutes, the solution was neutralized by addition of TFA (122 μL, 1578 μmol). The solution was diluted with DMSO and purified by ISCO RP-HPLC and upon lyophilization N-(4-((32S,35S)-15-(3-(2-((tert-butoxycarbonyl)amino)ethoxy)propanoyl)-1-carboxy-32-isopropyl-13,17,30,33-tetraoxo-35-(3-ureidopropyl)-3,6,9,21,24,27-hexaoxa-12,15,18,31,34-pentaazahexatriacontan-36-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (109 mg, 42% yield) was obtained. HRMS: M⁺=1843.9600, Rt=1.90 min (5 min acidic method).

### Synthesis of N-(4-((32S,35S)-15-(3-(2-((tert-butoxycarbonyl)amino)ethoxy)propanoyl)-1-carboxy-32-isopropyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

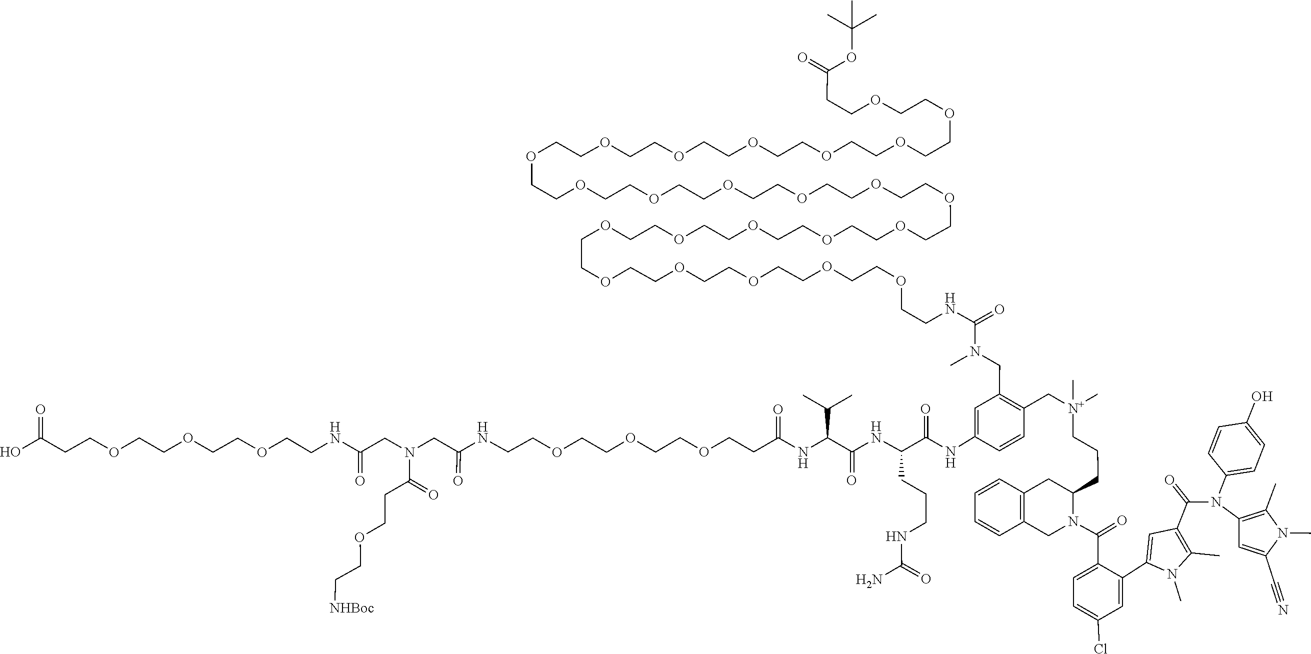 [SMILES: Cc1c(N(C(=O)c2cc(-c3cc(Cl)ccc3C(=O)N3Cc4ccccc4C[C@H]3CCC[N+](C)(C)Cc3ccc(NC(=O)[C@H](CCCNC(N)=O)NC(=O)[C@@H](NC(=O)CCOCCOCCOCCNC(=O)CN(CC(=O)NCCOCCOCCOCCC(=O)O)C(=O)CCOCCNC(=O)OC(C)(C)C)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)OC(C)(C)C)n(C)c2C)c2ccc(O)cc2)cc(C#N)n1C]

Following GENERAL PROCEDURE #1 using bis(4-nitrophenyl) carbonate (11.9 mg, 39 μmol), DIEA (98 μL, 560 μmol), tert-butyl 1-amino-3,6,9,12,15,18,21,24,27,30,33,36,39,42,45,48,51,54,57,60,63,66,69,72-tetracosaoxapentaheptacontan-75-oate (47.1 mg, 39 μmol) and then N-(4-((32S,35S)-15-(3-(2-((tert-butoxycarbonyl)amino)ethoxy)propanoyl)-1-carboxy-32-isopropyl-13,17,30,33-tetraoxo-35-(3-ureidopropyl)-3,6,9,21,24,27-hexaoxa-12,15,18,31,34-pentaazahexatriacontan-36-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (73.1 mg, 37 μmol), N-(4-((32S,35S)-15-(3-(2-((tert-butoxycarbonyl)amino)ethoxy)propanoyl)-1-carboxy-32-isopropyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (76 mg, 64%) was obtained. HRMS: M⁺=3071.6799, Rt=2.44 min (5 min acidic method).

### Synthesis of 1-(4-((6S,9S,43S,46S)-1-amino-26-(3-(2-((tert-butoxycarbonyl)amino)ethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,43-diisopropyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

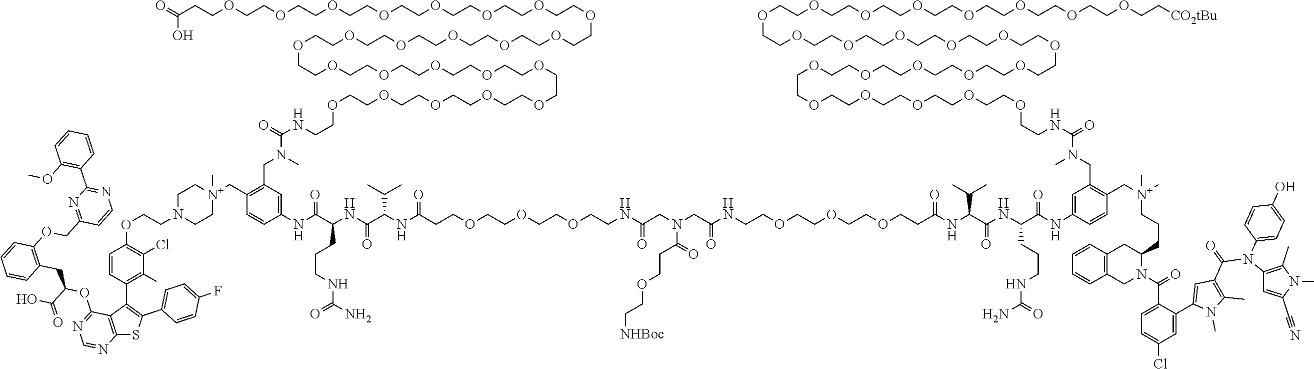

Following GENERAL PROCEDURE #4 using N-(4-((32S,35S)-15-(3-(2-((tert-butoxycarbonyl)amino)ethoxy)propanoyl)-1-carboxy-32-isopropyl-13,17,30,33-tetraoxo-35-(3-ureidopropyl)-3,6,9,21,24,27-hexaoxa-12,15,18,31,34-pentaazahexatriacontan-36-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (82 mg, 26 μmol), TSTU (7.3 mg, 24 μmol), DIEA (45 μL, 257 μmol) and then 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium TFA salt (72.3 mg, 27 μmol), 1-(4-((6S,9S,43S,46S)-1-amino-26-(3-(2-((tert-butoxycarbonyl)amino)ethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,43-diisopropyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[l2,3-d]pyrimidin-5-yl)-2-chloro-3-methylphenoxy)ethyl)-1-methylpiperazin-1-ium trifluoroacetate (65.6 mg, 44% yield) was obtained. HRMS: (M⁺²- H⁺)+=5503.8701, Rt=2.65 min (5 min acidic method).

### Synthesis of 1-(4-((6S,9S,43S,46S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L16-P2)

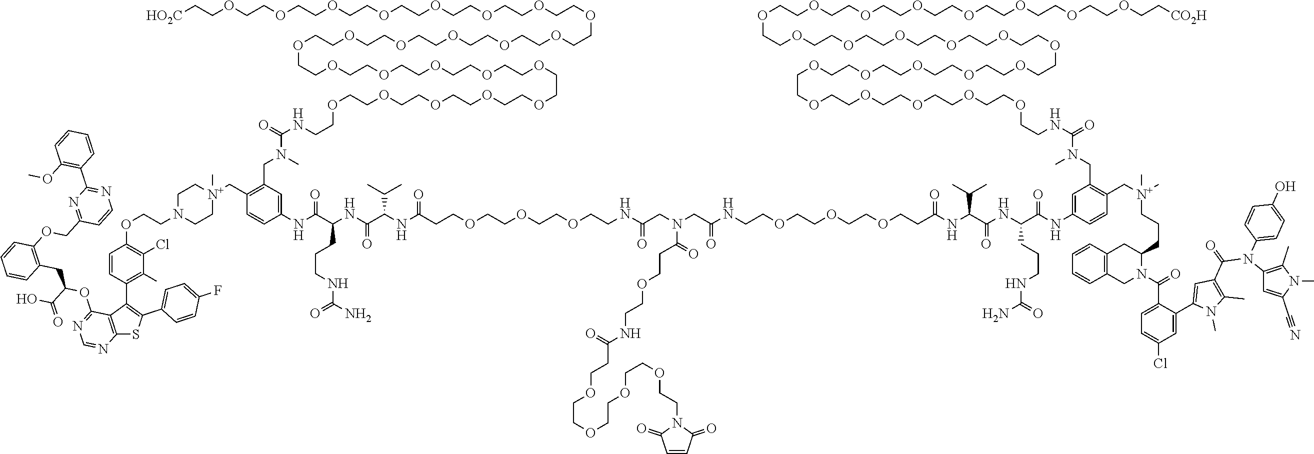

Following the GENERAL PROCEDURE #5 using 1-(4-((6S,9S,43S,46S)-1-amino-26-(3-(2-((tert-butoxycarbonyl)amino)ethoxy)propanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,43-diisopropyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (65.6 mg, 11 μmol), 2,5-dioxopyrrolidin-1-yl 1-(2,5-dioxo-2,5-dihydro-1H-pyrrol-l-yl)-3,6,9,12-tetraoxapentadecan-15-oate (10.1 mg, 23 μmol) and DIEA (40 μL, 229 μmol), 1-(4-((6S,9S,43S,46S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-26-(1-(2,5-dioxo-2,5-dihydro-1H-pyrrol-l-yl)-15-oxo-3,6,9,12,19-pentaoxa-16-azadocosan-22-oyl)-9,43-diisopropyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (52.5 mg, 77% yield) was obtained. HRMS: (M⁺²- H⁺)+=5674.8501, Rt=2.46 min (5 min acidic method).

### Synthesis of N-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

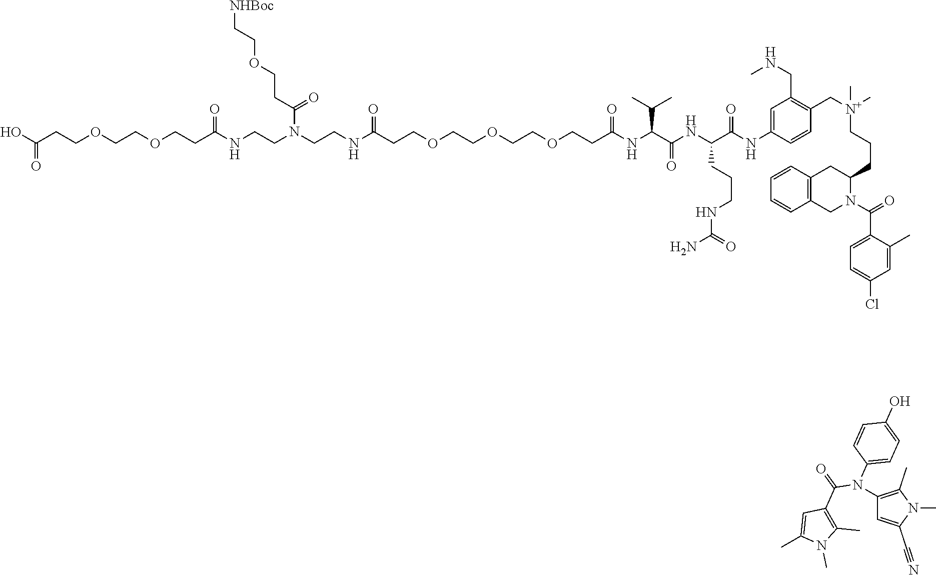

A solution of 17-(3-(2-((tert-butoxycarbonyl)amino)ethoxy)propanoyl)-4,13,21-trioxo-3,7,10,24,27,30-hexaoxa-14,17,20-triazatritriacontan-33-oic acid (153 mg, 200 μmol), HATU (80 mg, 211 μmol) and DIEA (314 μL, 1796 μmol) in DMF (5 mL) was stirred for 5 minutes and then N-(2-(((((9H-fluoren-9-yl)methoxy)carbonyl)(methyl)amino)methyl)-4-((S)-2-((S)-2-amino-3-methylbutanamido)-5-ureidopentanamido)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (288 mg, 200 μmol) was added. After stirring for 10 minutes, MeOH (2 mL) and 1N LiOH (2 mL, 2000 μmol) were added. After stirring for 15 minutes, acetic acid (228 μL, 3990 μmol) was added to neutralize, DMSO was added and the solution was purified by ISCO RP-HPLC. Upon lyophilization, N-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (278 mg, 68% yield) was obtained. HRMS: [(M⁺+H⁺)/2]⁺=914.9800, Rt=1.87 min (5 min acidic method).

### Synthesis of N-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

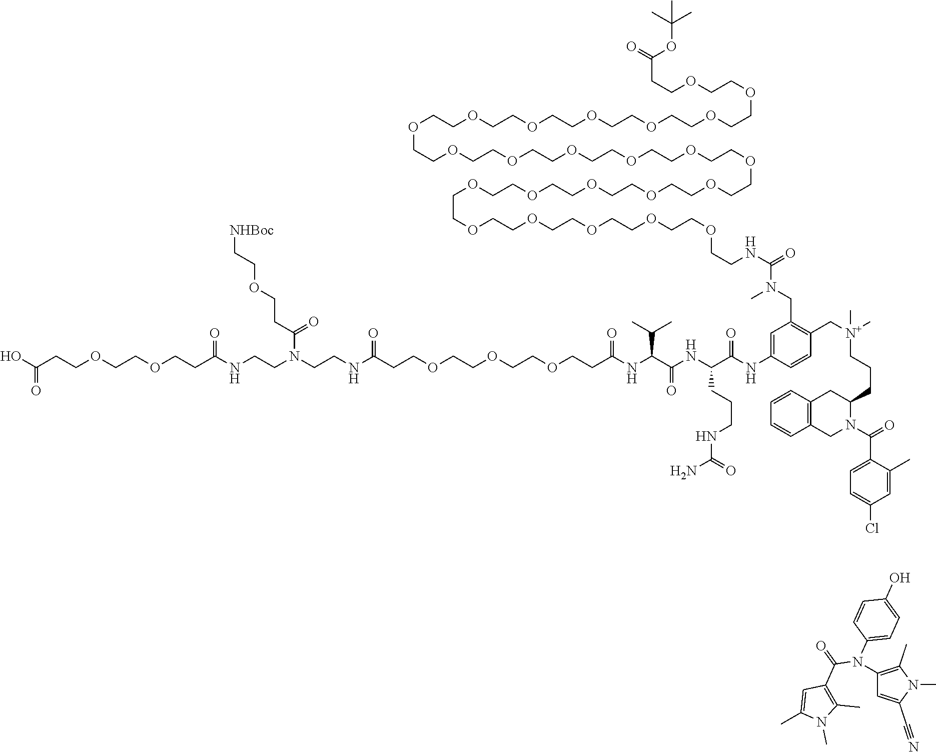

Following GENERAL PROCEDURE #1 using bis(4-nitrophenyl) carbonate (20 mg, 66 μmol), DIEA (55 μL, 316 μmol), tert-butyl 1-amino-3,6,9,12,15,18,21,24,27,30,33,36,39,42,45,48,51,54,57,60,63,66,69,72-tetracosaoxapentaheptacontan-75-oate (80 mg, 66 μmol) and then N-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (130 mg, 63 μmol) and additional DIEA (110 p L, 632 mmol), N-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (152 mg, 76%) was obtained. HRMS: M⁺=3055.6799, Rt=2.42 min (5 min acidic method).

### Synthesis of 1-(4-((6R,9R,42S,45S)-1-amino-27-(3-(2-((tert-butoxycarbonyl)amino)ethoxy)propanoyl)-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

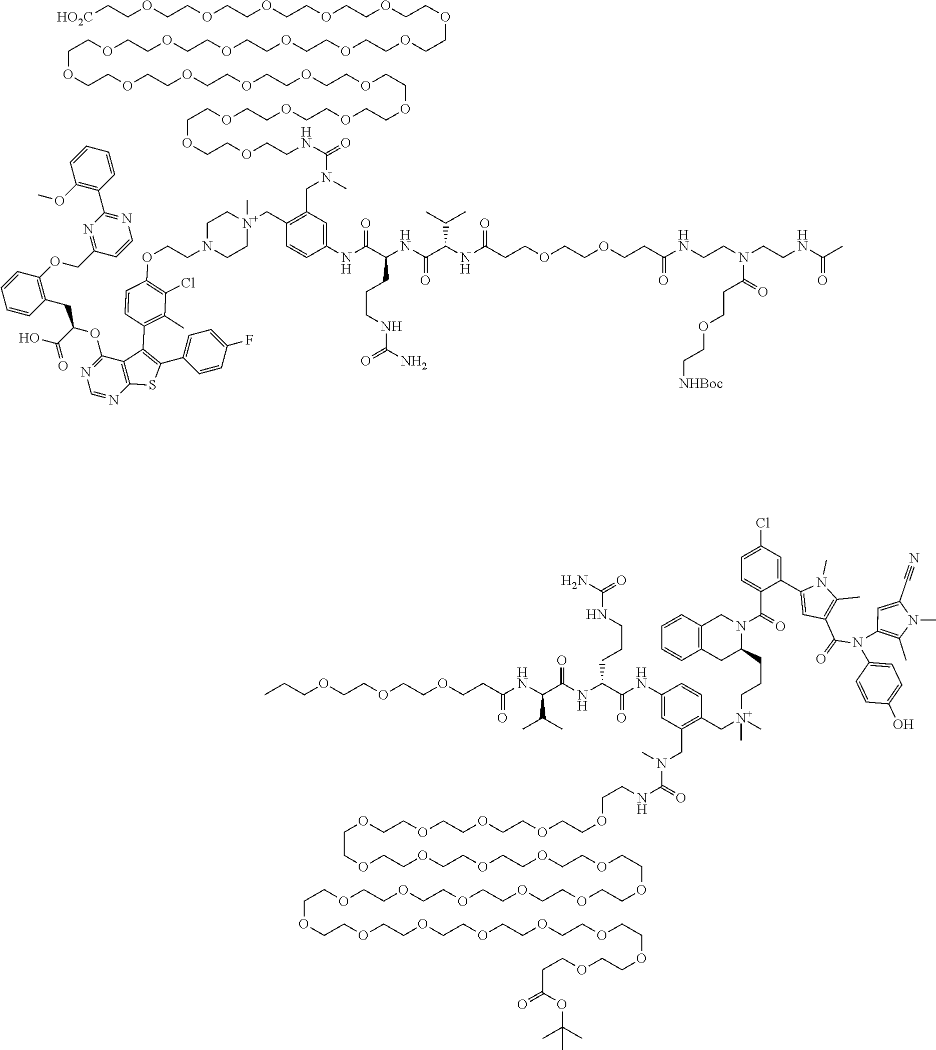

Following GENERAL PROCEDURE #4 using N-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (138 mg, 44 μmol), TSTU (13.1 mg, 44 μmol), DIEA (38 pj L, 215 μmol) and then 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium TFA salt (133 mg, 50 μmol) and DIEA (76 μL, 430 μmol), 1-(4-((6R,9R,42S,45S)-1-amino-27-(3-(2-((tert-butoxycarbonyl)amino)ethoxy)propanoyl)-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (149 mg, 54% yield) was obtained. HRMS: (M⁺²- H⁺)+=5487.8398, Rt=2.63 min (5 min acidic method).

### Synthesis of 1-(4-((6S,9S,42S,45S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-27-(1-(2,5-dioxo-2,5-dihydro-1H-pyrrol-1-yl)-15-oxo-3,6,9,12,19-pentaoxa-16-azadocosan-22-oyl)-9,42-diisopropyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L17-P2)

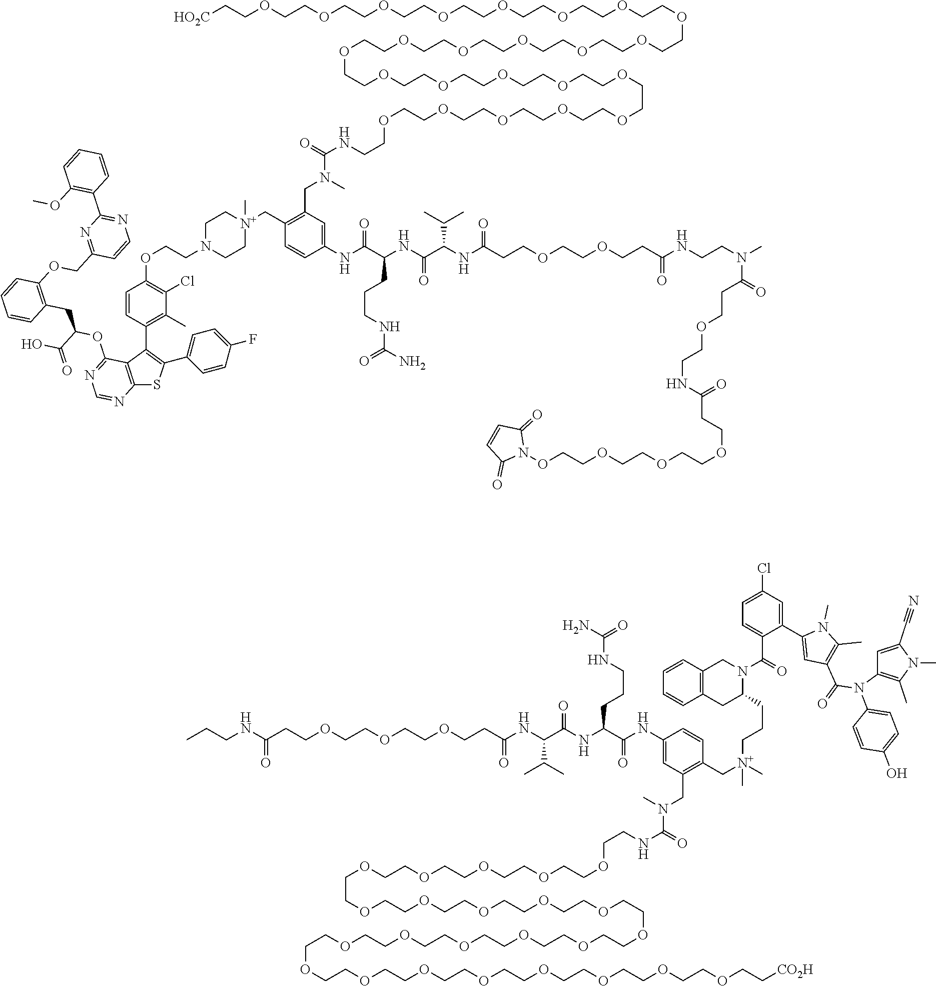

Following the GENERAL PROCEDURE #5 using 1-(4-((6R,9R,42S,45S)-1-amino-27-(3-(2-((tert-butoxycarbonyl)amino)ethoxy)propanoyl)-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,42-diisopropyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (149 mg, 26 μmol), 2,5-dioxopyrrolidin-1-yl 1-(2,5-dioxo-2,5-dihydro-1H-pyrrol-l-yl)-3,6,9,12-tetraoxapentadecan-15-oate (13 mg, 29 μmol) and DIEA (26 p L, 149 μmol), 1-(4-((6S,9S,42S,45S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-27-(1-(2,5-dioxo-2,5-dihydro-1H-pyrrol-l-yl)-15-oxo-3,6,9,12,19-pentaoxa-16-azadocosan-22-oyl)-9,42-diisopropyl-1,8,11,23,31,40,43-heptaoxo-45-(3-ureidopropyl)-14,17,20,34,37-pentaoxa-2,7,10,24,27,30,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (63 mg, 41% yield) was obtained. HRMS: (M⁺²- H⁺)+=5658.8398, Rt=2.46 min (5 min acidic method).

### Synthesis of 4-(4-((2S,5S)-23-(3-(2-((tert-butoxycarbonyl)amino)ethoxy)propanoyl)-5-isopropyl-4,7,19,27,36-pentaoxo-2-(3-ureidopropyl)-10,13,16,30,33,37-hexaoxa-3,6,20,23,26-pentaazatetracont-39-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

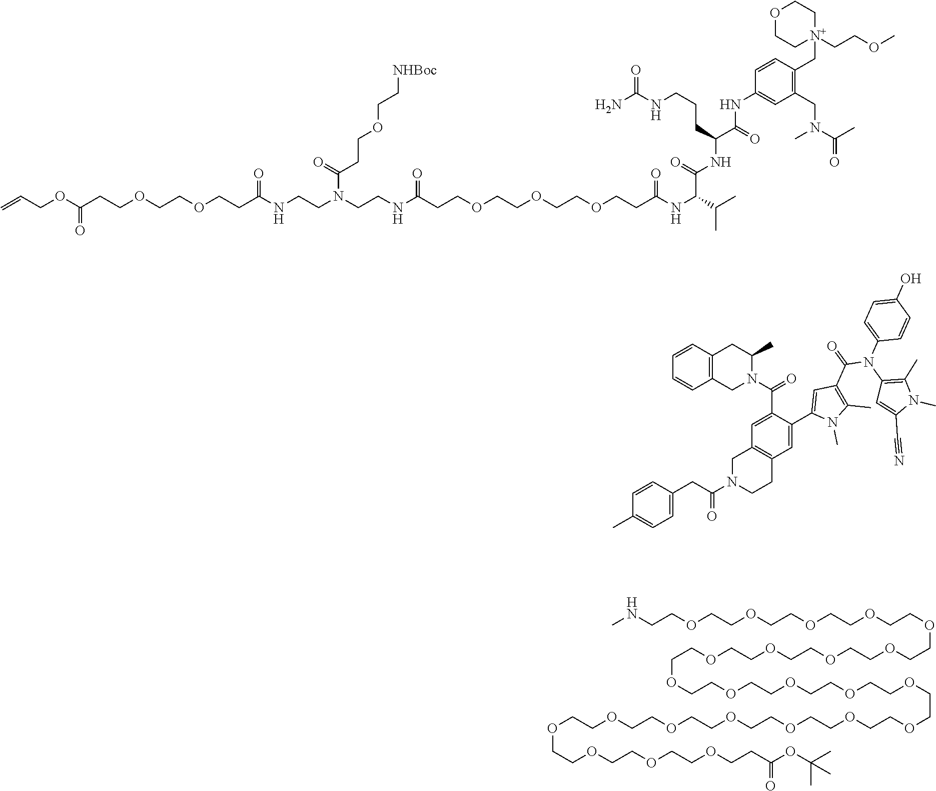

Following GENERAL PROCEDURE #3 using 4-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium (209 mg, 75.7 μmol) and 17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid (67.8 mg, 87 μmol), 4-(4-((2S,5S)-23-(3-(2-((tert-butoxycarbonyl)amino)ethoxy)propanoyl)-5-isopropyl-4,7,19,27,36-pentaoxo-2-(3-ureidopropyl)-10,13,16,30,33,37-hexaoxa-3,6,20,23,26-pentaazatetracont-39-enamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (216 mg, 78%) was obtained. HRMS: M⁺=3292.8401, Rt=2.51 min (5 min acidic method).

### Synthesis of 4-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

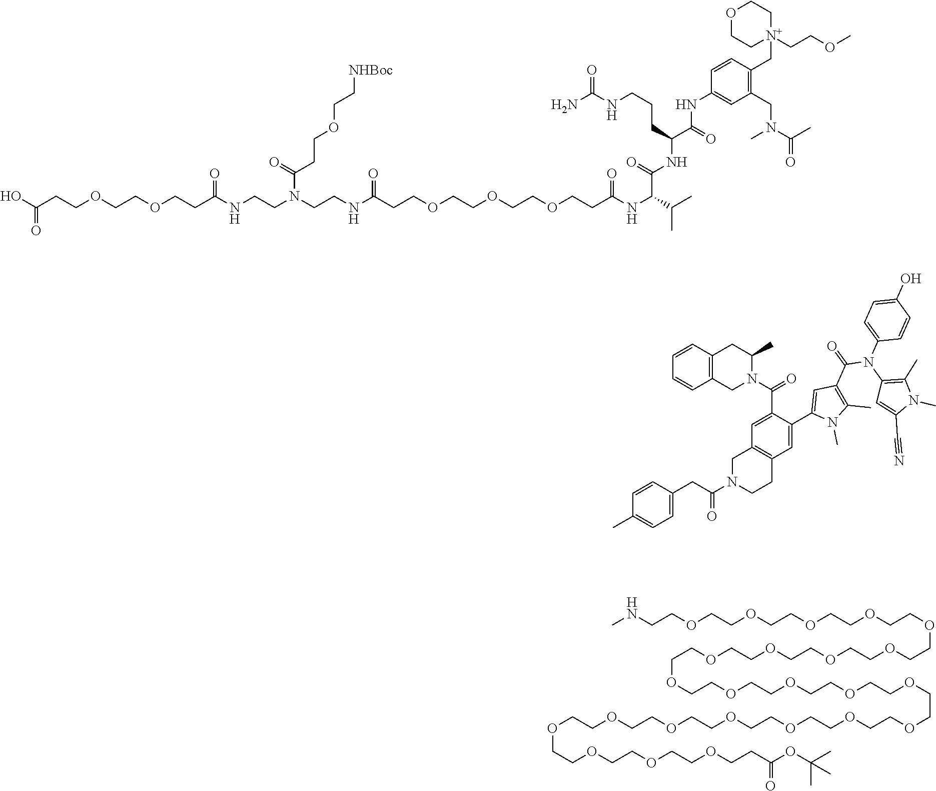

Following GENERAL PROCEDURE #2 using 4-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (204 mg, 59.8 μmol), morpholine (25.8 μL, 299 μmol) and tetrakis(triphenylphosphine)palladium (10.4 mg, 8.98 μmol), 4-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (163 mg, 81% yield) was obtained. HRMS: M⁺=3252.8101, Rt=2.37 min (5 min acidic method).

### Synthesis of 4-(4-((6S,9S,42S,45S)-1-amino-24-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

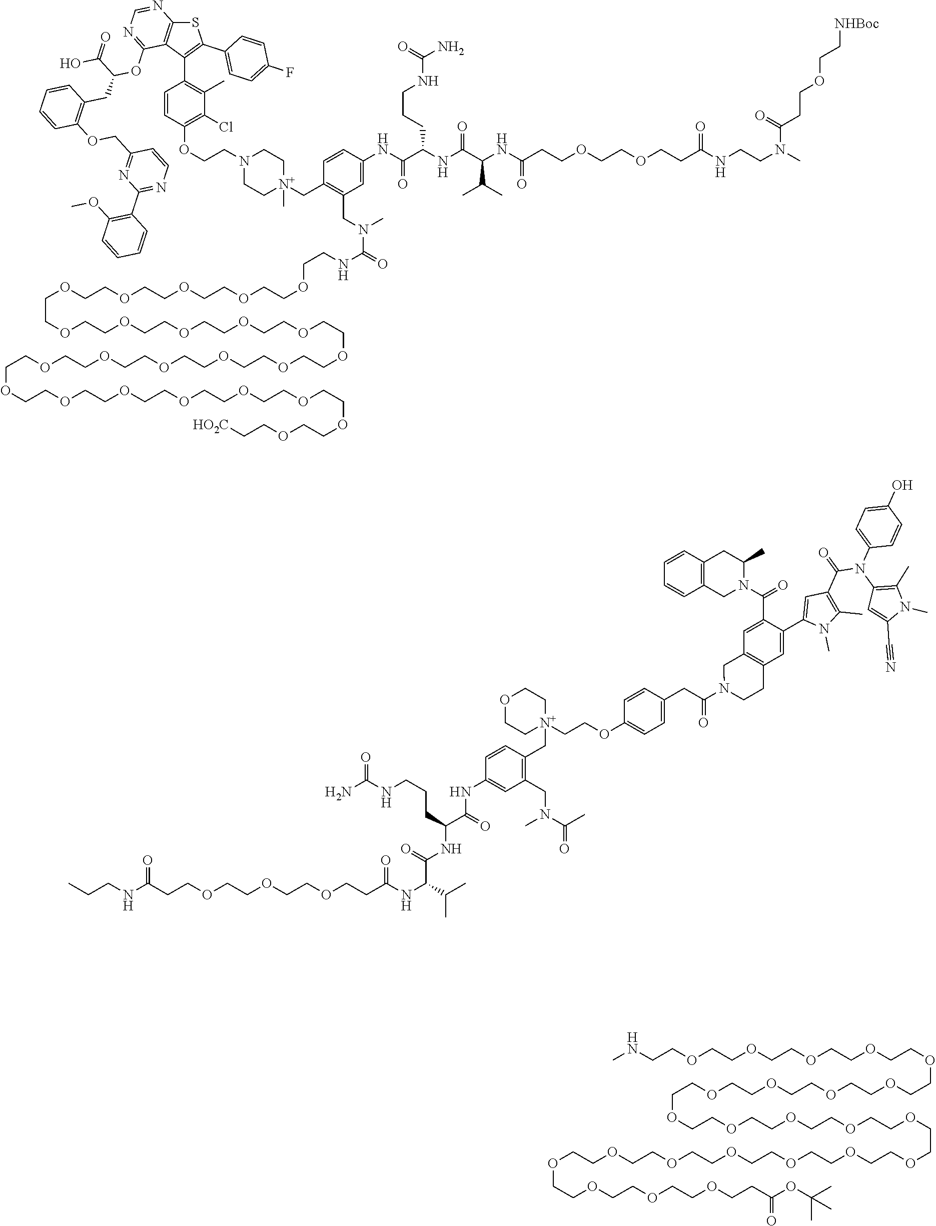

Following GENERAL PROCEDURE #4 using 4-(4-((2S,5S)-23-(3-(2-((tert-butoxycarbonyl)amino)ethoxy)propanoyl)-35-carboxy-5-isopropyl-4,7,19,27-tetraoxo-2-(3-ureidopropyl)-10,13,16,30,33-pentaoxa-3,6,20,23,26-pentaazapentatriacontanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (163 mg, 48.4 μmol), TSTU (14.6 mg, 48.4 μmol), 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium TFA salt (149.2 mg, 55.6 μmol) and DIEA (84 μL, 484 μmol), 4-(4-((6S,9S,42S,45S)-1-amino-24-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (138 mg, 48% yield) was obtained. HRMS: (M⁺²-H⁺)+=5694.9902, Rt=2.62 min (5 min acidic method).

### Synthesis of 4-(4-((6S,9S,42S,45S)-1-amino-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-24-(1-(2,5-dioxo-2,5-dihydro-1H-pyrrol-1-yl)-15-oxo-3,6,9,12,19-pentaoxa-16-azadocosan-22-oyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (P1-L17-P4)

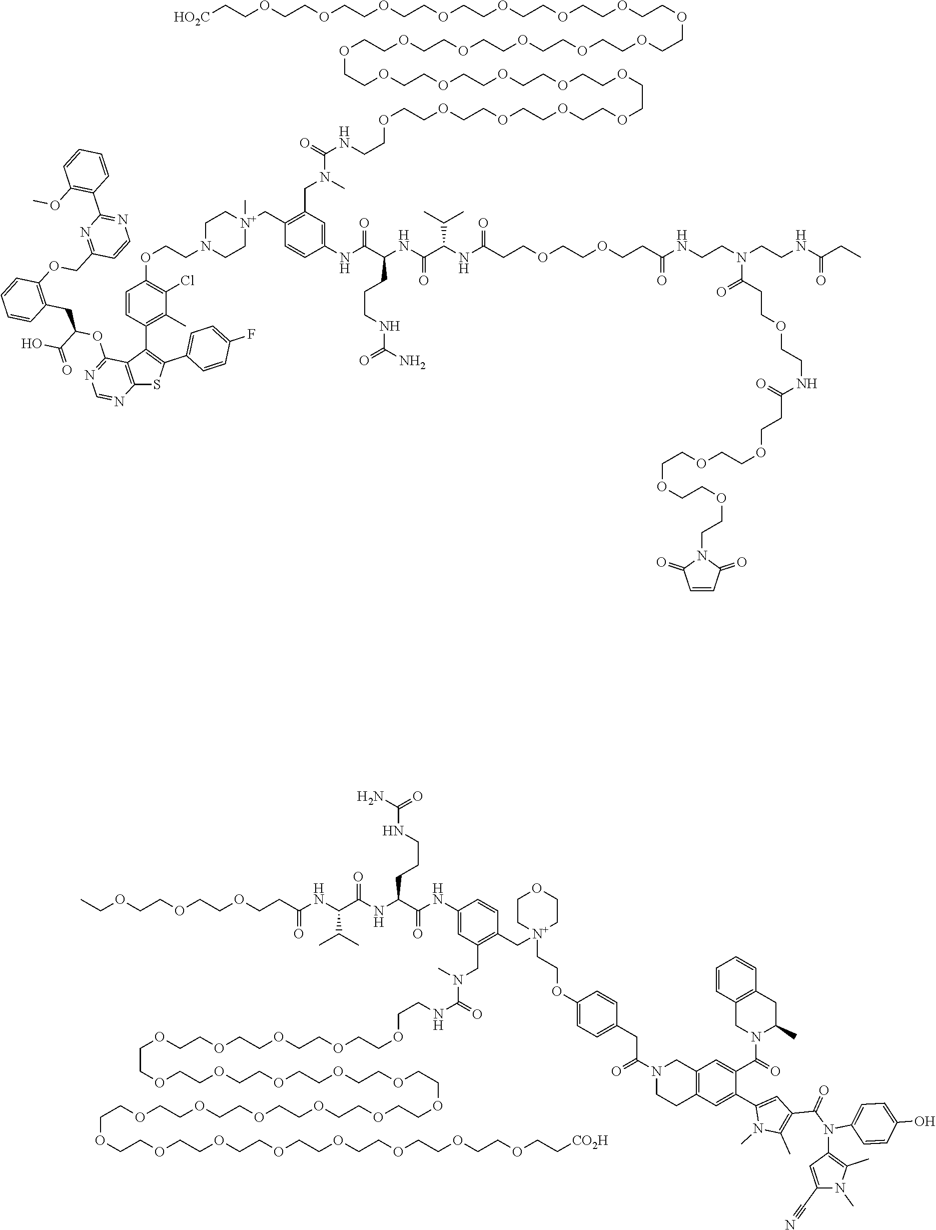

Following GENERAL PROCEDURE #5 using 4-(4-((6S,9S,42S,45S)-1-amino-24-(3-(2-((tert-butoxycarbonyl)amino)ethoxy)propanoyl)-6-((4-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (133 mg, 22.5 μmol), 2,5-dioxopyrrolidin-1-yl 1-(2,5-dioxo-2,5-dihydro-1H-pyrrol-l-yl)-3,6,9,12-tetraoxapentadecan-15-oate (14.9 mg, 33.7 μmol) and DIEA (39 p L, 225 μmol),, 4-(4-((6S,9S,42S,45S)-1-amino-6-((4-((4-(2-(4-(4-((R)-1-carboxy-2-(2-((2-(2- methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-24-(1-(2,5-dioxo-2,5-dihydro-1H-pyrrol-l-yl)-15-oxo-3,6,9,12,19-pentaoxa-16-azadocosan-22-oyl)-9,42-diisopropyl-1,8,11,20,28,40,43-heptaoxo-45-(3-ureidopropyl)-14,17,31,34,37-pentaoxa-2,7,10,21,24,27,41,44-octaazahexatetracontan-46-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (102 mg, 74% yield) was obtained. HRMS: (M⁺²-H⁺)+=5855.9902, Rt=2.47 min (5 min acidic method).

### Synthesis of N-(4-((17S,30S,33S)-17-(4-((tert-butoxycarbonyl)amino)butyl)-1-carboxy-30-isopropyl-15,18,28,31-tetraoxo-33-(3-ureidopropyl)-3,6,9,12,22,25-hexaoxa-16,19,29,32-tetraazatetratriacontan-34-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate

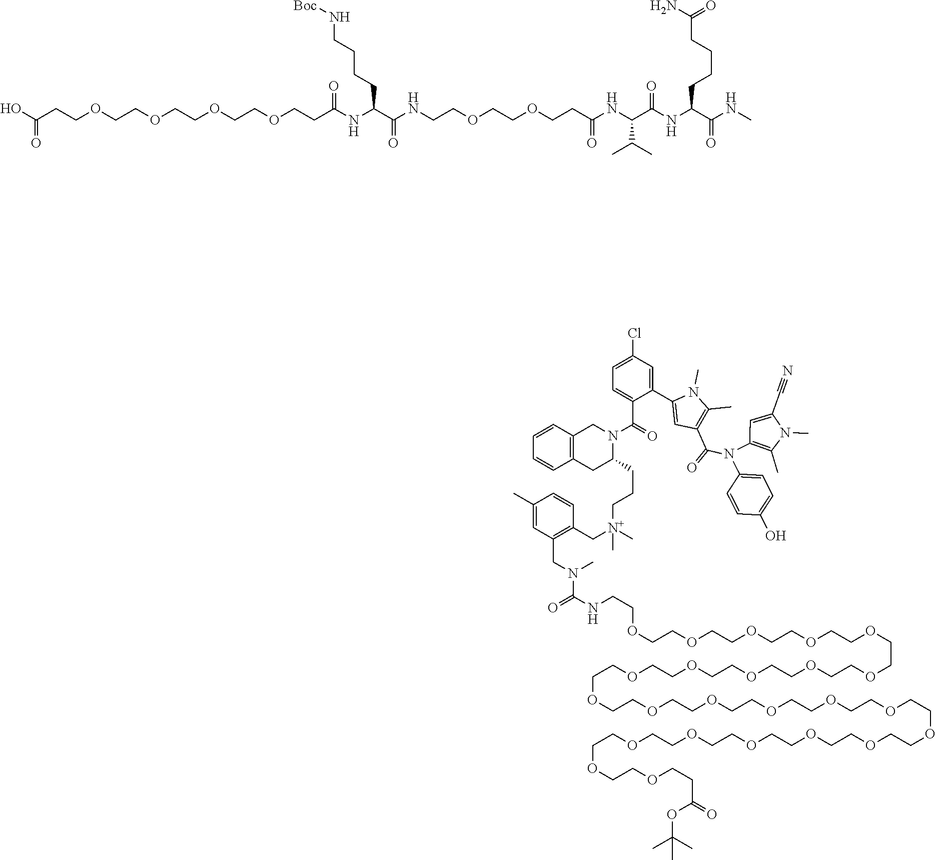

To a solution of N-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (225 mg, 88 μmol) and (S)-20-(4-((tert-butoxycarbonyl)amino)butyl)-3,18,21-trioxo-2,6,9,12,15,25,28-heptaoxa-19,22-diazahentriacontan-31-oic acid (80 mg, 110 μmol) in DMF (0.9 mL) was added DIEA (77 μL, 439 μmol) and HATU (40 mg, 105 μmol). After stirring for 1.5 hours, MeOH (0.5 mL) and 1N LiOH (877 μL, 877 μmol) were added. After stirring for 30 minutes, the solution was diluted with DMSO (10 mL), cooled in an ice bath, neutralized by addition of 1N HCl (1.4 mL, 1400 μmol). The solution was purified by ISCO RP-HPLC and upon lyophilization N-(4-((17S,30S,33S)-17-(4-((tert-butoxycarbonyl)amino)butyl)-1-carboxy-30-isopropyl-15,18,28,31-tetraoxo-33-(3-ureidopropyl)-3,6,9,12,22,25-hexaoxa-16,19,29,32-tetraazatetratriacontan-34-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (183 mg, 67% yield) was obtained. HRMS: M⁺=2998.6599, Rt=2.66 min (5 min acidic method).

### Synthesis of 1-(4-((6R,9R,22S,41S,44S)-1-amino-22-(4-((tert-butoxycarbonyl)amino)butyl)-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,41-diisopropyl-1,8,11,21,24,39,42-heptaoxo-44-(3-ureidopropyl)-14,17,27,30,33,36-hexaoxa-2,7,10,20,23,40,43-heptaazapentatetracontan-45-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

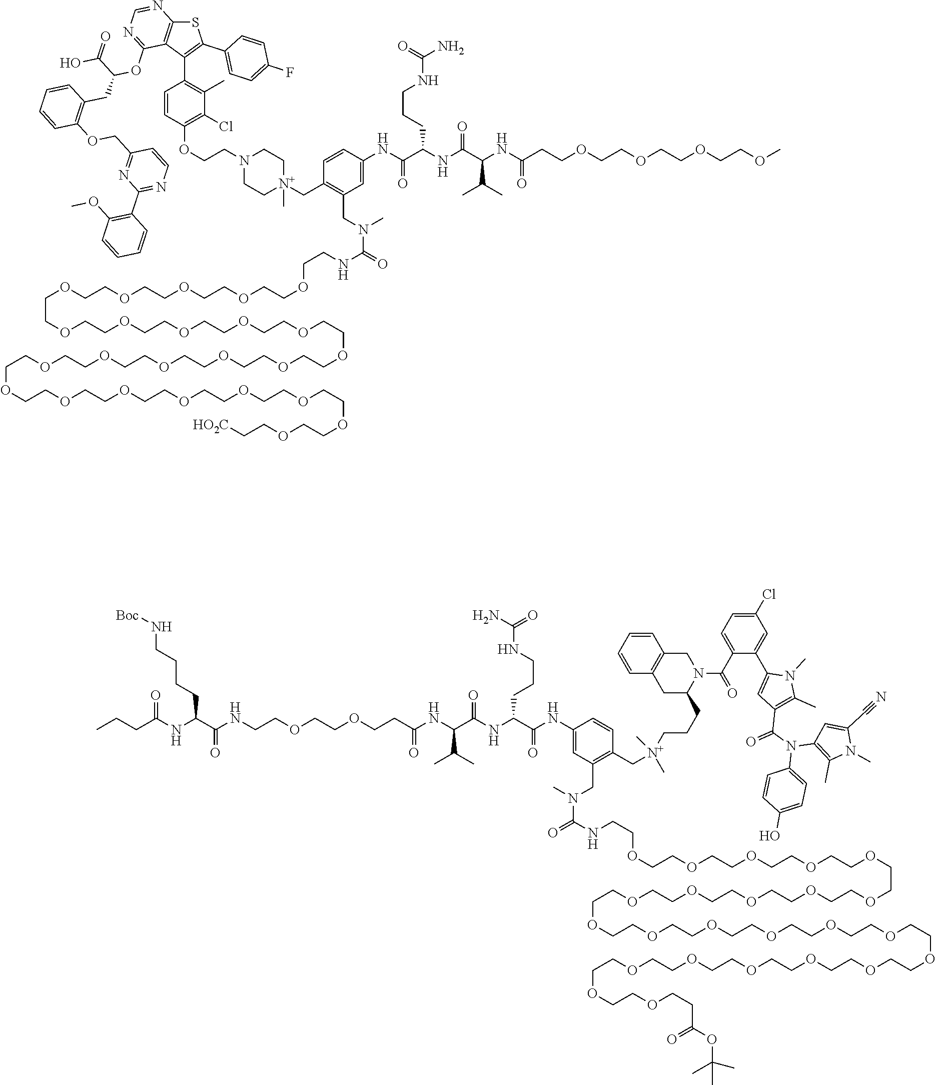

Following GENERAL PROCEDURE #4 using N-(4-((17S,30S,33S)-17-(4-((tert-butoxycarbonyl)amino)butyl)-1-carboxy-30-isopropyl-15,18,28,31-tetraoxo-33-(3-ureidopropyl)-3,6,9,12,22,25-hexaoxa-16,19,29,32-tetraazatetratriacontan-34-amido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (117 mg, 38 μmol), TSTU (11.3 mg, 38 μmol), DIEA (66 μL, 376 μmol) and then 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium TFA salt (108 mg, 40 μmol) and DIEA (66 μL, 376 μmol), 1-(4-((6R,9R,22S,41S,44S)-1-amino-22-(4-((tert-butoxycarbonyl)amino)butyl)-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,41-diisopropyl-1,8,11,21,24,39,42-heptaoxo-44-(3-ureidopropyl)-14,17,27,30,33,36-hexaoxa-2,7,10,20,23,40,43-heptaazapentatetracontan-45-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (104.5 mg, 49% yield) was obtained. HRMS: (M⁺²-H⁺)+=5430.8398, Rt=2.66 min (5 min acidic method).

### Synthesis of 1-(4-((6R,9R,22S,41S,44S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-22-(1-(2,5-dioxo-2,5-dihydro-1H-pyrrol-1-yl)-15-oxo-3,6,9,12-tetraoxa-16-azaicosan-20-yl)-9,41-diisopropyl-1,8,11,21,24,39,42-heptaoxo-44-(3-ureidopropyl)-14,17,27,30,33,36-hexaoxa-2,7,10,20,23,40,43-heptaazapentatetracontan-45-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L18-P2)

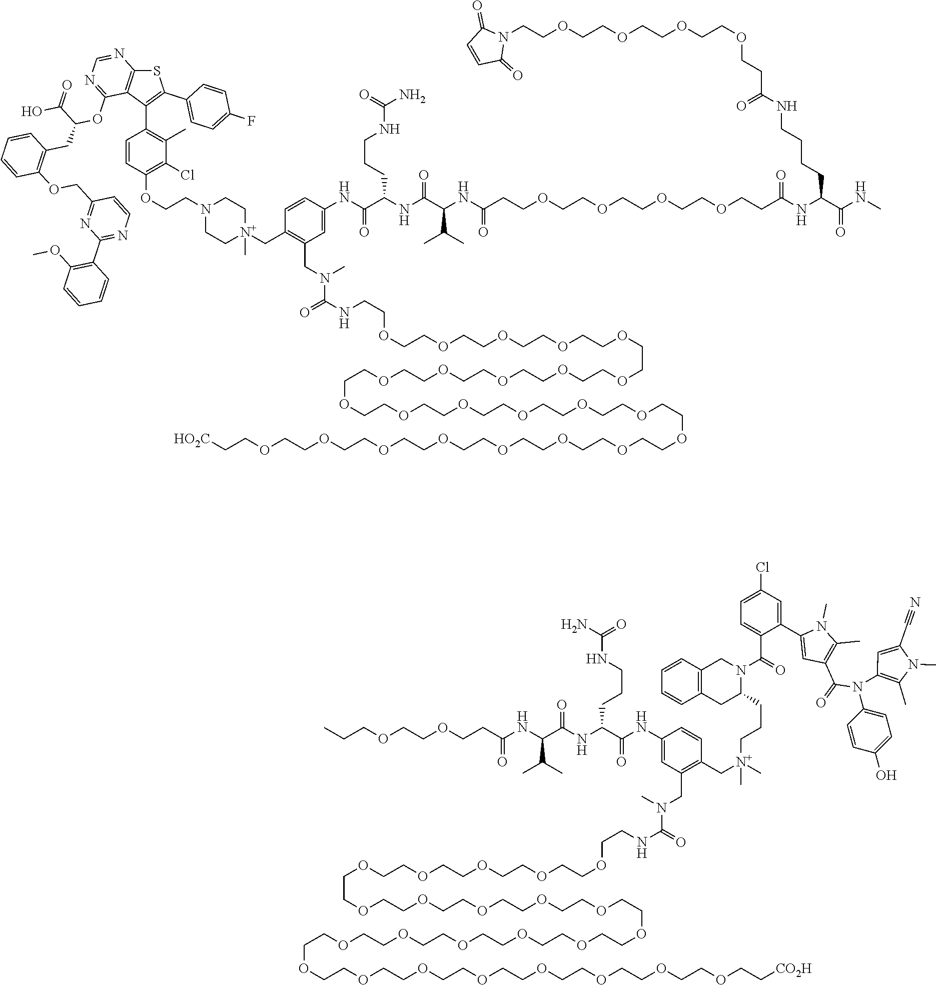

Following the GENERAL PROCEDURE #5 using 1-(4-((6R,9R,22S,41S,44S)-1-amino-22-(4-((tert-butoxycarbonyl)amino)butyl)-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)phenyl)carbamoyl)-9,41-diisopropyl-1,8,11,21,24,39,42-heptaoxo-44-(3-ureidopropyl)-14,17,27,30,33,36-hexaoxa-2,7,10,20,23,40,43-heptaazapentatetracontan-45-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-m trifluoroacetate (105 mg, 18 μmol), 2,5-dioxopyrrolidin-1-yl 1-(2,5-dioxo-2,5-dihydro-1H-pyrrol-1-yl)-3,6,9,12-tetraoxapentadecan-15-oate (12.2 mg, 28 μmol) and DIEA (23.8 mg, 184 μmol), 1-(4-((6R,9R,22S,41S,44S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-22-(1-(2,5-dioxo-2,5-dihydro-1H-pyrrol-1-yl)-15-oxo-3,6,9,12-tetraoxa-16-azaicosan-20-yl)-9,41-diisopropyl-1,8,11,21,24,39,42-heptaoxo-44-(3-ureidopropyl)-14,17,27,30,33,36-hexaoxa-2,7,10,20,23,40,43-heptaazapentatetracontan-45-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (85.6 mg, 80% yield) was obtained. HRMS: (M⁺²-H⁺)⁺=5601.8198, Rt=2.47 min (5 min acidic method).

### Synthesis of 1,1′-(((((2S,2'S)-2,2′-(((2S,2'S)-2,2′-((((((8-(3-(2-aminoethoxy)propanoyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

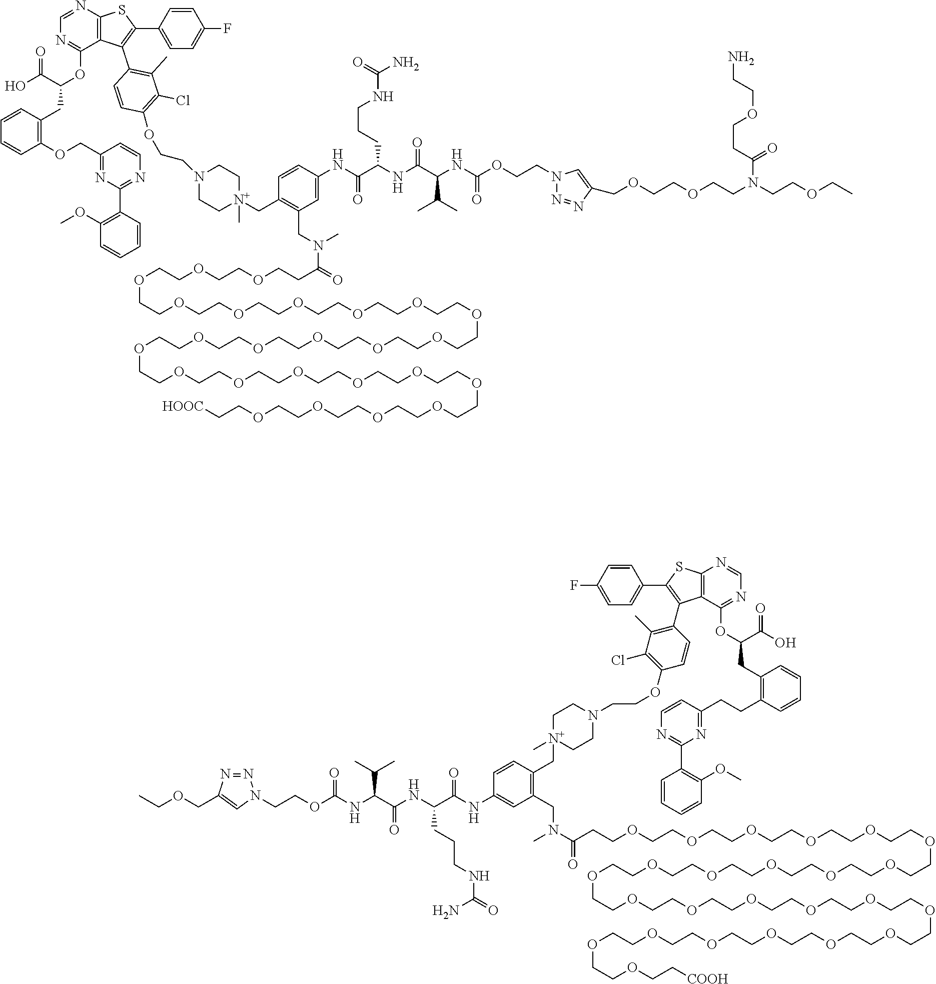

GENERAL PROCEDURE #7: A mixture of 3-(2-aminoethoxy)-N,N-bis(2-(2-(prop-2-yn-1-yloxy)ethoxy)ethyl)propanamide trifluoroacetate (3.9 mg, 7.8 μmol) and 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[l2,3-d]pyrimidin-5-yl)-2-chloro-3-methylphenoxy)ethyl)-1-methylpiperazin-1-ium trifluoroacetate (52.8 mg, 20 μmol) in DMSO (1.9 mL) was degassed by pulling vacuum and purging to balloon of N₂ (repeated 3×). Sodium Absorbate 32 mg/mL in H₂O solution (174 μL, 28 μmol) was then added followed by degassing (3×). CuSO₄ pentahydrate 16 mg/mL in H₂O solution (147 μL, 9.4 μmol) was then added followed by degassing. The mixture was stirred under N₂ balloon for 2 hours at which time the solution was directly purified by ISCO RP-HPLC. Upon lyophilization, 1,1′-(((((2S,2'S)-2,2′-(((2S,2'S)-2,2′-((((((8-(3-(2-aminoethoxy)propanoyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (24.8 mg, 54% yield) was obtained. HRMS: (M⁺²- H⁺)+=5569.6602, Rt=2.53 min (5 min acidic method).

### Synthesis of 1,1′-(((((2S,2'S)-2,2′-(((2S,2'S)-2,2′-((((((8-(1-(2,5-dioxo-2,5-dihydro-1H-pyrrol-1-yl)-15-oxo-3,6,9,12,19-pentaoxa-16-azadocosan-22-oyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L20-P1)

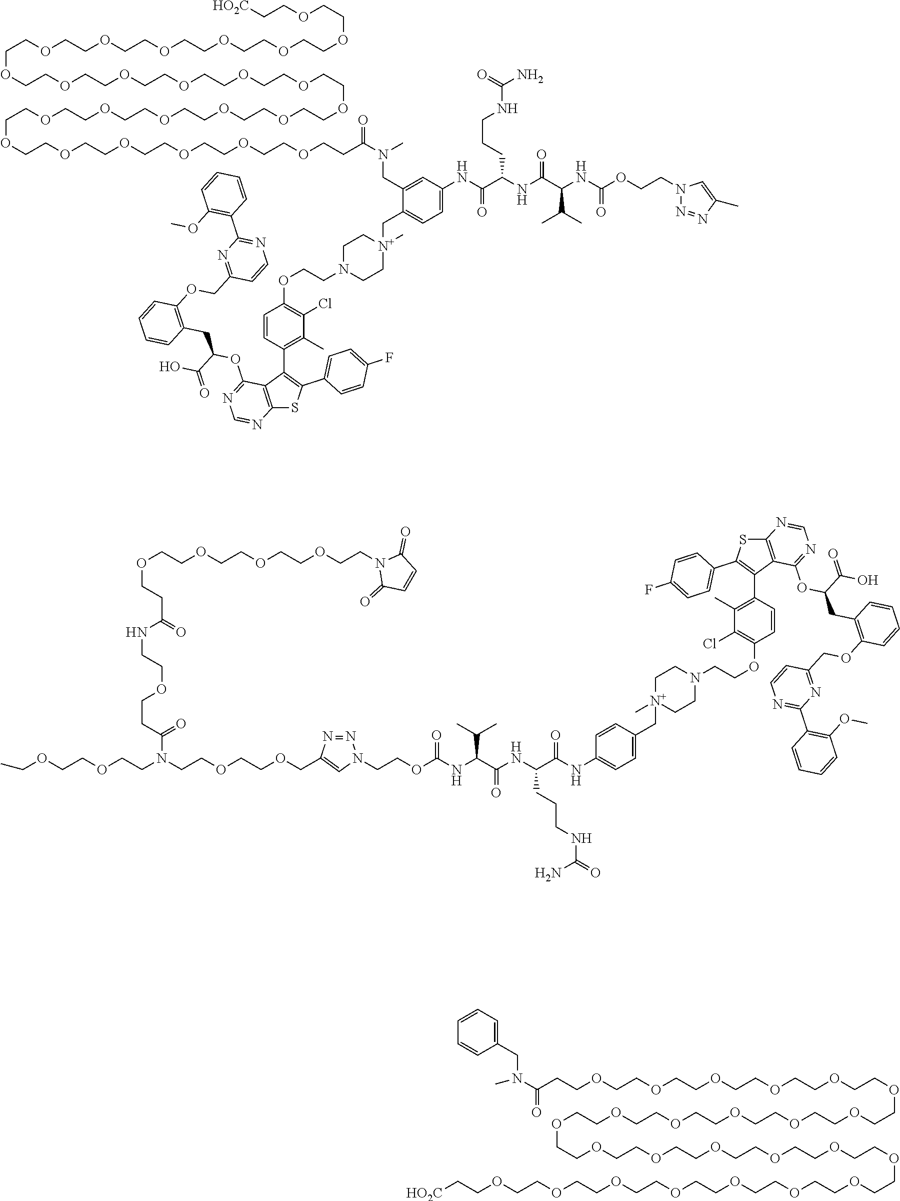

Following the second part of GENERAL PROCEDURE #5 using 1,1′-(((((2S,2'S)-2,2′-(((2S,2'S)-2,2′-((((((8-(3-(2-aminoethoxy)propanoyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (25 mg, 4.2 μmol), 2,5-dioxopyrrolidin-1-yl 1-(2,5-dioxo-2,5-dihydro-1H-pyrrol-l-yl)-3,6,9,12-tetraoxapentadecan-15-oate (5.6 mg, 13 μmol) and DIEA (11 p L, 63 μmol), 1,1′-(((((2S,2'S)-2,2′-(((2S,2'S)-2,2′-((((((8-(1-(2,5-dioxo-2,5-dihydro-1H-pyrrol-l-yl)-15-oxo-3,6,9,12,19-pentaoxa-16-azadocosan-22-oyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (20 mg, 76% yield) was obtained. HRMS: (M⁺²- H⁺)+=5896.7700, Rt=2.53 min (5 min acidic method).

### Synthesis of 1,1′-(((((2S,2'S)-2,2′-(((2S,2'S)-2,2′-(((((((S)-16-amino-15-oxo-2,5,8,11-tetraoxa-14-azaheptadecane-1,17-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

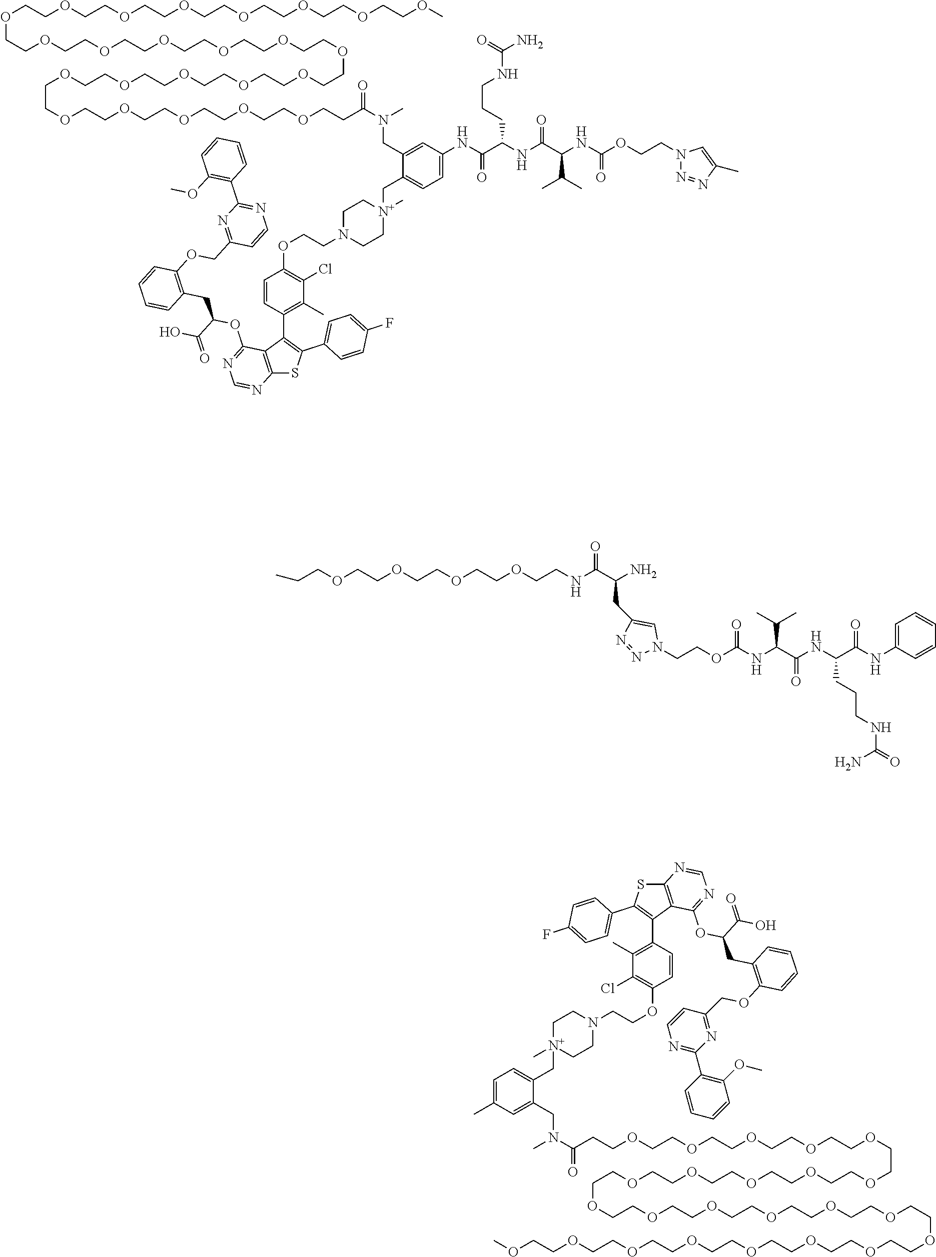

Following the GENERAL PROCEDURE #7 using (S)-2-amino-N-(3,6,9,12-tetraoxapentadec-14-yn-1-yl)pent-4-ynamide (4 mg, 12 μmol) and 1-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (79 mg, 32 μmol) in DMSO (1 mL) was degassed by pulling vacuum and purging to balloon of N₂ (repeated 3×). Sodium Absorbate 32 mg/mL in H₂O solution (228 μL, 37 μmol) was then added followed by degassing (3×). CuSO₄ pentahydrate 16 mg/mL in H₂O solution (191 μL, 12 μmol) was then added followed by degassing. The mixture was stirred under N₂ balloon for 2 hours at which time the solution was directly purified by ISCO RP-HPLC. Upon lyophilization, 1,1′-(((((2S,2'S)-2,2′-(((2S,2'S)-2,2′-(((((((S)-16-amino-15-oxo-2,5,8,11-tetraoxa-14-azaheptadecane-1,17-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (33.3 mg, 51% yield) was obtained. HRMS: (M⁺²- H⁺)+=5307.5098, Rt=2.41 min (5 min acidic method).

### Synthesis of 1,1′-(((((2S,2'S)-2,2′-(((2S,2'S)-2,2′-(((((((S)-16-(1-(2,5-dioxo-2,5-dihydro-1H-pyrrol-1-yl)-3,6,9,12,15,18,21,24-octaoxaheptacosan-27-amido)-15-oxo-2,5,8,11-tetraoxa-14-azaheptadecane-1,17-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L21-P1)

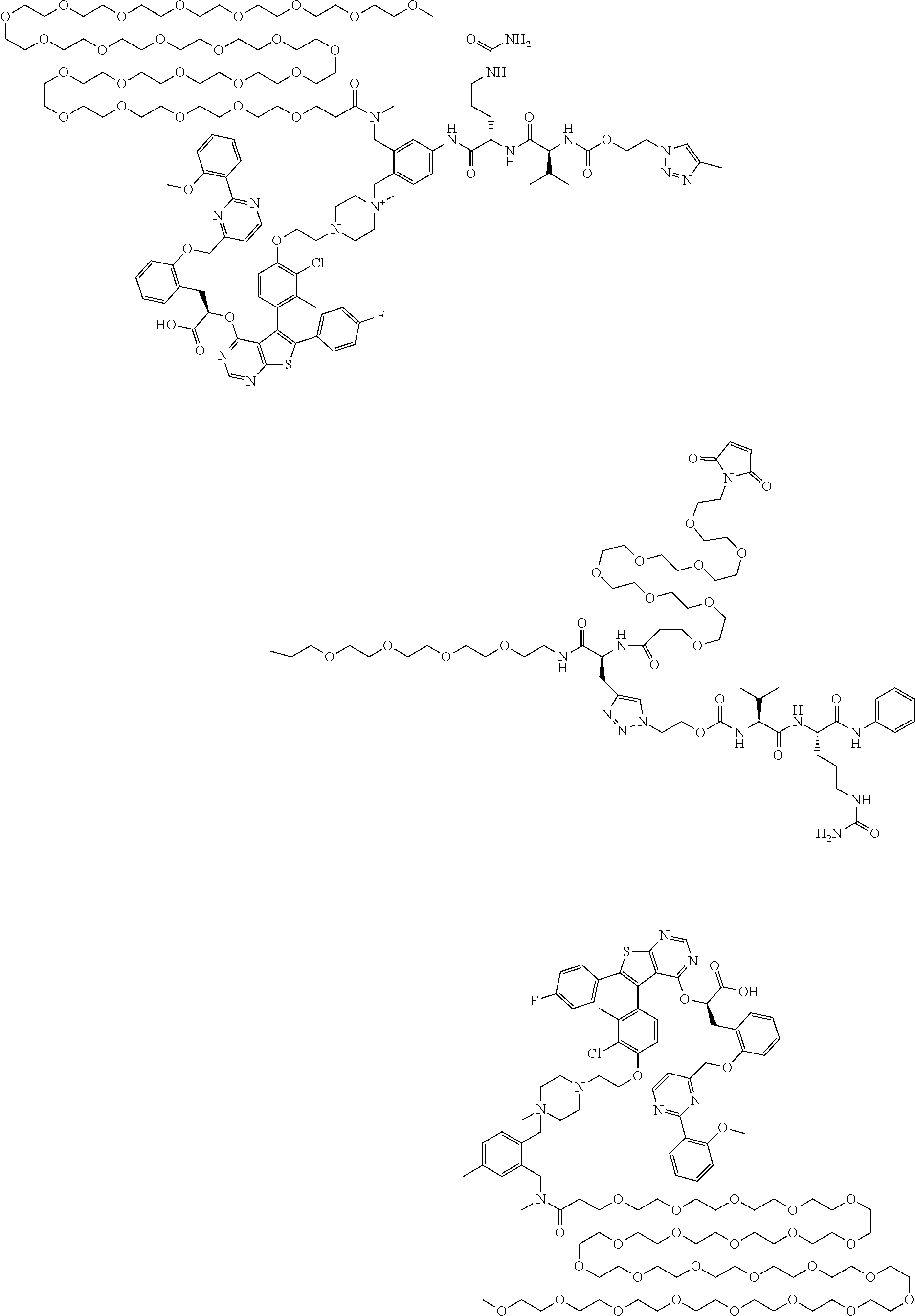

Following the second part of GENERAL PROCEDURE #5 using 1,1′-(((((2S,2'S)-2,2′-(((2S,2'S)-2,2′-(((((((S)-16-amino-15-oxo-2,5,8,11-tetraoxa-14-azaheptadecane-1,17-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (33.3 mg, 6.3 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (19.4 mg, 31 μmol) and DIEA (22 p L, 125 μmol), 1,1′-(((((2S,2'S)-2,2′-(((2S,2'S)-2,2′-(((((((S)-16-(1-(2,5-dioxo-2,5-dihydro-1H-pyrrol-l-yl)-3,6,9,12,15,18,21,24-octaoxaheptacosan-27-amido)-15-oxo-2,5, 8,11-tetraoxa-14-azaheptadecane-1,17-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (18.5 mg, 48% yield) was obtained. HRMS: (M⁺²- H⁺)+=5810.6602, Rt=2.57 min (5 min acidic method).

### Synthesis of 1,1′-(((((2S,2'S)-2,2′-(((2S,2'S)-2,2′-((((((8-(3-aminopropanoyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate

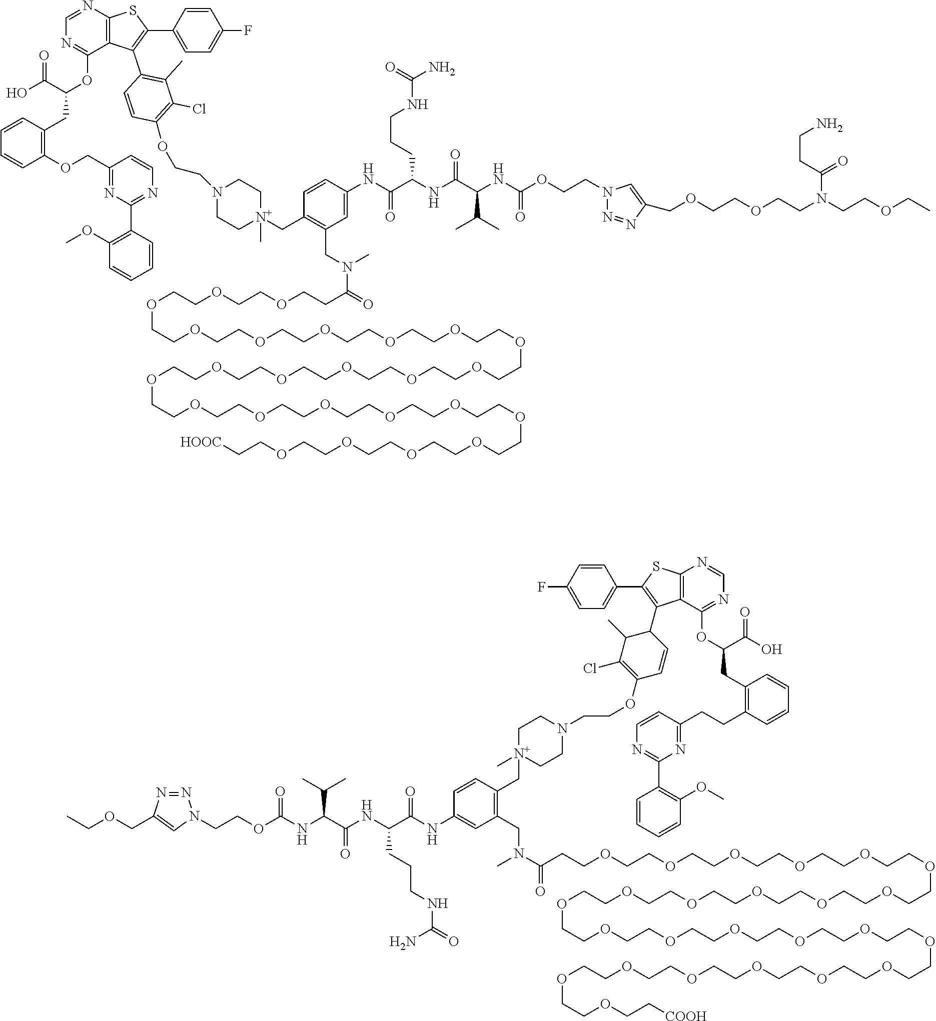

Following the GENERAL PROCEDURE #7 using 3-amino-N,N-bis(2-(2-(prop-2-yn-1-yloxy)ethoxy)ethyl)propanamide (3.5 mg, 7.7 μmol), 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (45 mg, 17 μmol), sodium ascorbate 16 mg/mL in H₂O solution (286 μL, 23 μmol) and CuSO₄ pentahydrate 4 mg/mL H₂O solution (288 μL, 4.6 μmol), 1,1′-(((((2S,2'S)-2,2′-(((2S,2'S)-2,2′-((((((8-(3-aminopropanoyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (38 mg, 80% yield) was obtained. HRMS: (M⁺²- H⁺)+=5525.6401, Rt=2.56 min (5 min acidic method).

### Synthesis of 1,1′-(((((2S,2'S)-2,2′-(((2S,2'S)-2,2′-((((((8-(1-(2,5-dioxo-2,5-dihydro-1H-pyrrol-1-yl)-15-oxo-3,6,9,12-tetraoxa-16-azanonadecan-l9-oyl)-2,5,11,14-tetraoxa-8-azapentadecane-1,15-diyl)bis(1H-1,2,3-triazole-4,1l-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L22-P1)

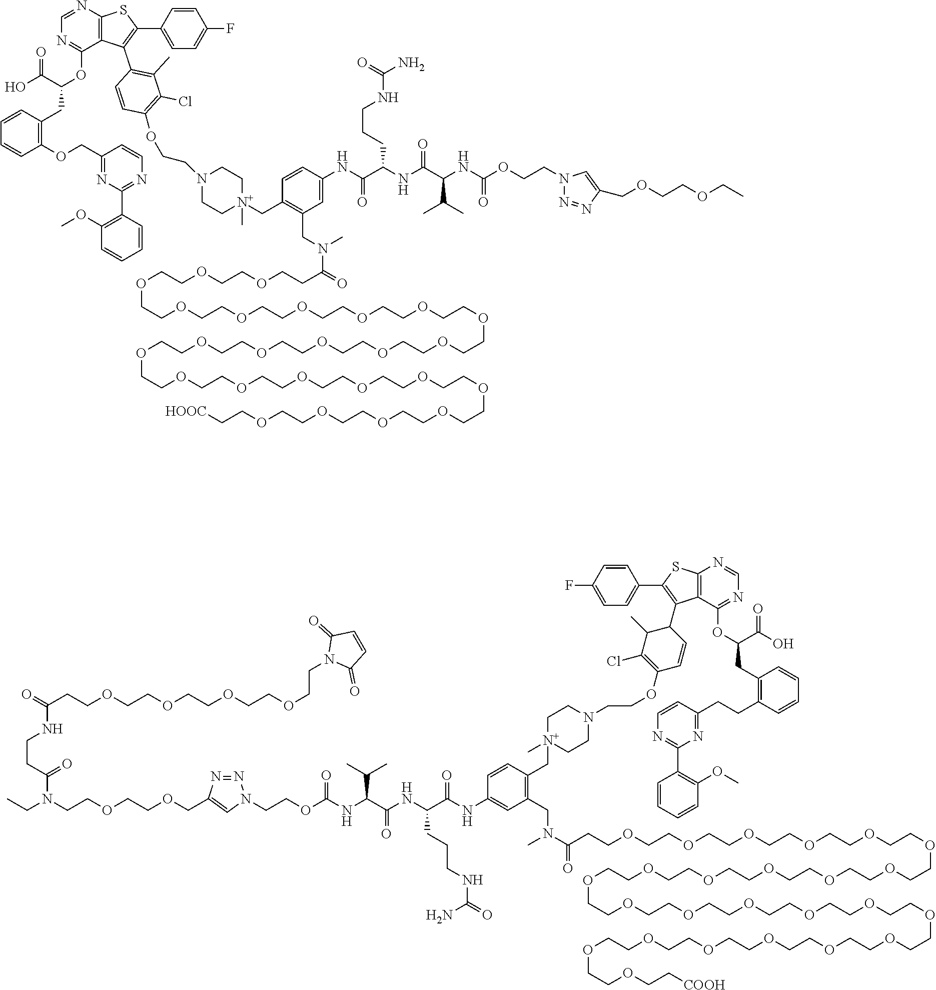

Following the second part of GENERAL PROCEDURE #5 using 1,1′-(((((2S,2'S)-2,2′-(((2S,2'S)-2,2′-((((((8-(3-aminopropanoyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (38 mg, 6.5 μmol), 2,5-dioxopyrrolidin-1-yl 1-(2,5-dioxo-2,5-dihydro-1H-pyrrol-l-yl)-3,6,9,12-tetraoxapentadecan-15-oate (4.3 mg, 9.7 μmol) and DIEA (23 μL, 129 μmol), 1,1′-(((((2S,2'S)-2,2′-(((2S,2'S)-2,2′-((((((8-(1-(2,5-dioxo-2,5-dihydro-1H-pyrrol-l-yl)-15-oxo-3,6,9,12-tetraoxa-16-azanonadecan-19-oyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (21.5 mg, 52% yield) was obtained. HRMS: (M⁺²- H⁺)+=5852.7900, Rt=2.67 min (5 min acidic method).

### Synthesis of 1,1′-(((((2S,2'S)-2,2′-(((2S,2'S)-2,2′-((((((8-(1-(2,5-dioxo-2,5-dihydro-1H-pyrrol-1-yl)-15-oxo-3,6,9,12,19-pentaoxa-16-azadocosan-22-oyl)-2,5,11,14-tetraoxa-8-azapentadecane-1,15-diyl)bis(1H-1,2,3-triazole-4,1l-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L23-P1)

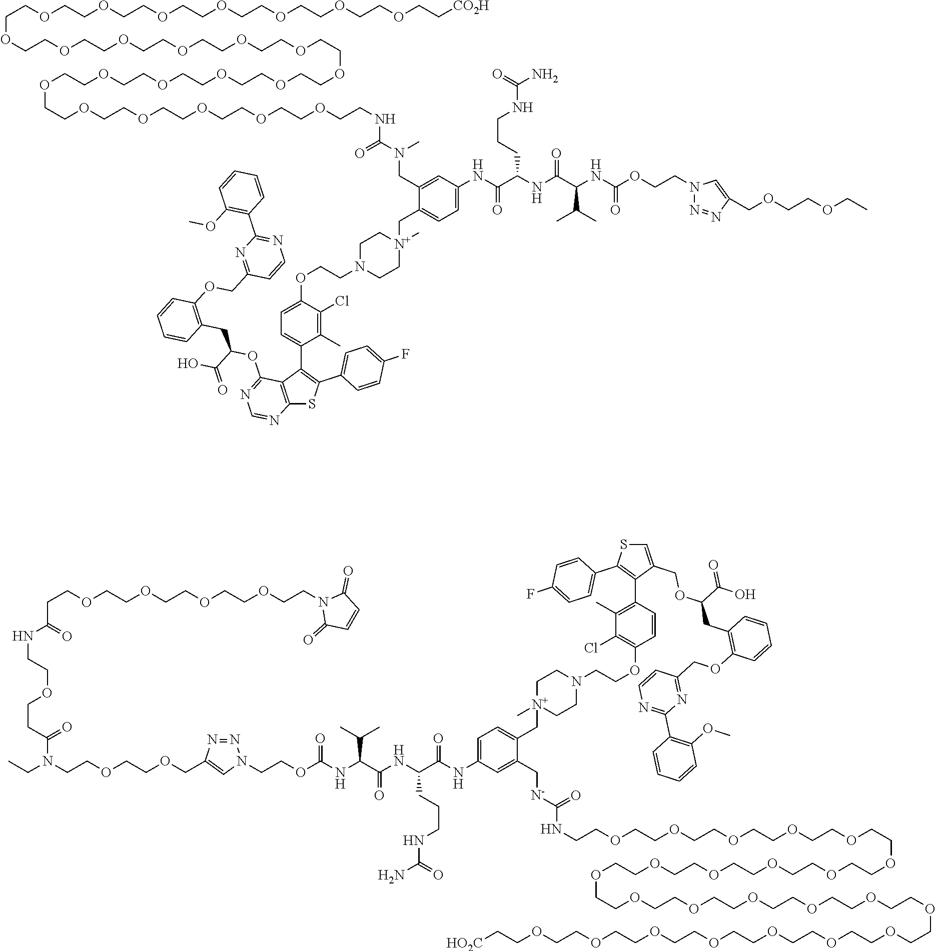

Following the GENERAL PROCEDURE #7 using 3-(2-aminoethoxy)-N,N-bis(2-(2-(prop-2-yn-1-yloxy)ethoxy)ethyl)propanamide TFA salt (4.7 mg, 9.5 μmol), 1-(4-((S)-2-((S)-2-(((2-azidoethoxy)carbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (53 mg, 20 μmol), sodium absorbate 16 mg/mL in H₂O solution (367 μL, 30 μmol) and CuSO₄ pentahydrate 4 mg/mL H₂O solution (370 μL, 5.9 μmol), bisclick product (21 mg, 39% yield) was obtained. HRMS: ((M⁺²- H⁺)+=5511.6299, Rt=2.39 min (5 min acidic method).

Following the second part of GENERAL PROCEDURE #5 using bisclick product (21 mg, 3.6 μmol), 2,5-dioxopyrrolidin-1-yl 1-(2,5-dioxo-2,5-dihydro-1H-pyrrol-1-yl)-3,6,9,12-tetraoxapentadecan-15-oate (6 mg, 14 μmol) and DIEA (xx p L, 86 μmol), 1,1′-(((((2S,2'S)-2,2′-(((2S,2'S)-2,2′-((((((8-(1-(2,5-dioxo-2,5-dihydro-1H-pyrrol-1-yl)-15-oxo-3,6,9,12,19-pentaoxa-16-azadocosan-22-oyl)-2,5,11,14-tetraoxa-8-azapentadecane-1,15-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (14.5 mg, 62% yield) was obtained. HRMS: (M⁺²- H⁺)+=5838.7300, Rt=2.53 min (5 min acidic method).

### Synthesis of 1,1′-(((((2S,2'S)-2,2′-(((2S,2'S)-2,2′-((((((11-(3-(2-((tert-butoxycarbonyl)amino)ethoxy)propanoyl)-2,5,8,14,17,20-hexaoxa-11-azahenicosane-1,21-diyl)bis(1H-1,2,3-triazole-4,1-diyl))bis(ethane-2,1-diyl))bis(oxy))bis(formyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium) trifluoroacetate

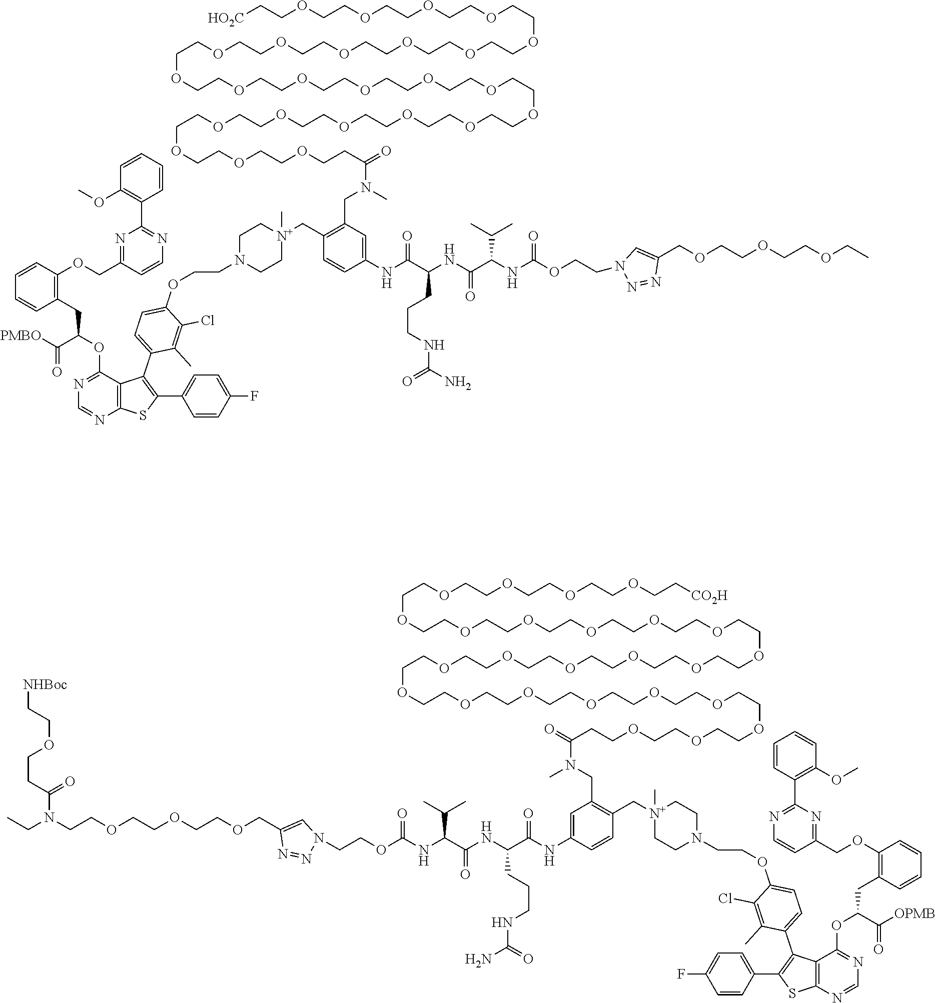

Following the GENERAL PROCEDURE #7 using tert-butyl (6-oxo-7-(2-(2-(2-(prop-2-yn-1-yloxy)ethoxy)ethoxy)ethyl)-3,10,13,16-tetraoxa-7-azanonadec-18-yn-1-yl)carbamate (6.0 mg, 10.5 μmol), 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (77 mg, 27 μmol), sodium ascorbate 16 mg/mL in H₂O solution (389 μL, 31 μmol) and CuSO₄ pentahydrate 4 mg/mL H₂O solution (654 μL, 10.5 μmol), 1,1′-(((((2S,2'S)-2,2′-(((2S,2'S)-2,2′-((((((11-(3-(2-((tert-butoxycarbonyl)amino)ethoxy)propanoyl)-2,5,8,14,17,20-hexaoxa-11-azahenicosane-1,21-diyl)bis(1H-1,2,3-triazole-4,1-diyl))bis(ethane-2,1-diyl))bis(oxy))bis(formyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium) trifluoroacetate was obtained. HRMS: (M⁺²- H⁺)+=5997.8838, Rt=2.97 min (5 min acidic method).

### Synthesis of 1,1′-(((((2S,2'S)-2,2′-(((2S,2'S)-2,2′-((((((11-(1-(2,5-dioxo-2,5-dihydro-1H-pyrrol-l-yl)-15-oxo-3,6,9,12,19-pentaoxa-16-azadocosan-22-oyl)-2,5,8,14,17,20-hexaoxa-11-azahenicosane-1,21-diyl)bis(1H-1,2,3-triazole-4,1l-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (P1-L24-P1)

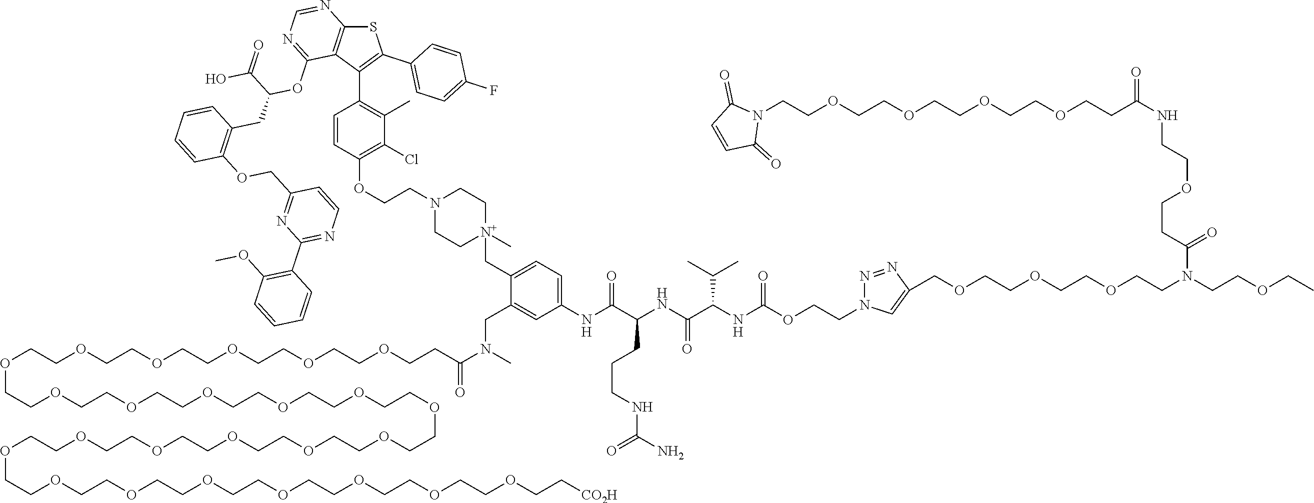 [SMILES: CCOCCN(CCOCCOCCOCc1cn(CCOC(=O)N[C@H](C(=O)N[C@@H](CCCNC(N)=O)C(=O)Nc2ccc(C[N+]3(C)CCN(CCOc4ccc(-c5c(-c6ccc(F)cc6)sc6ncnc(O[C@H](Cc7ccccc7OCc7ccnc(-c8ccccc8OC)n7)C(=O)O)c56)c(C)c4Cl)CC3)c(CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)c2)C(C)C)nn1)C(=O)CCOCCNC(=O)CCOCCOCCOCCOCCN1C(=O)C=CC1=O]

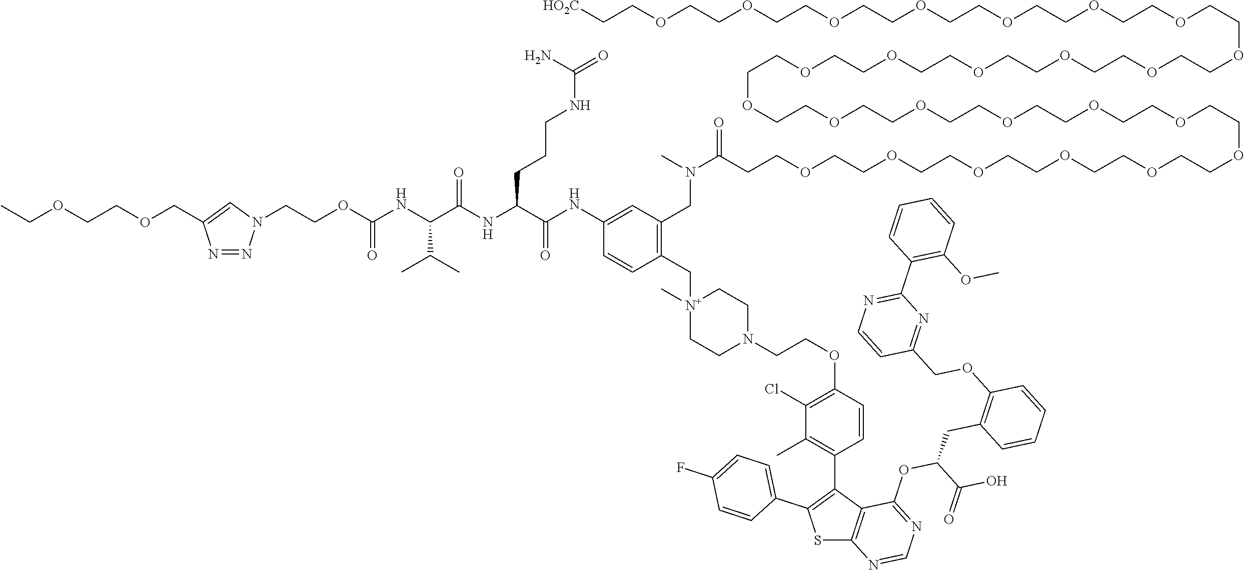 [SMILES: CCOCCOCc1cn(CCOC(=O)N[C@H](C(=O)N[C@@H](CCCNC(N)=O)C(=O)Nc2ccc(C[N+]3(C)CCN(CCOc4ccc(-c5c(-c6ccc(F)cc6)sc6ncnc(O[C@H](Cc7ccccc7OCc7ccnc(-c8ccccc8OC)n7)C(=O)O)c56)c(C)c4Cl)CC3)c(CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)c2)C(C)C)nn1]

Following the GENERAL PROCEDURE #5 using 1,1′-(((((2S,2'S)-2,2′-(((2S,2'S)-2,2′-((((((11-(3-(2-((tert-butoxycarbonyl)amino)ethoxy)propanoyl)-2,5,8,14,17,20-hexaoxa-11-azahenicosane-1,21-diyl)bis(1H-1,2,3-triazole-4,1-diyl))bis(ethane-2,1-diyl))bis(oxy))bis(formyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium) trifluoroacetate (assumed 10.5 μmol), 2,5-dioxopyrrolidin-1-yl 1-(2,5-dioxo-2,5-dihydro-1H-pyrrol-l-yl)-3,6,9,12-tetraoxapentadecan-15-oate (9.3 mg, 21 μmol) and DIEA (13.6 mg, 105 μmol), 1,1′-(((((2S,2'S)-2,2′-(((2S,2'S)-2,2′-((((((11-(1-(2,5-dioxo-2,5-dihydro-1H-pyrrol-1-yl)-15-oxo-3,6,9,12,19-pentaoxa-16-azadocosan-22-oyl)-2,5, 8,14,17,20-hexaoxa-11-azahenicosane-1,21-diyl)bis(1H-1,2,3-triazole-4,1-diyl))bis(ethane-2,1-diyl))bis(oxy))bis(carbonyl))bis(azanediyl))bis(3-methylbutanoyl))bis(azanediyl))bis(5-ureidopentanoyl))bis(azanediyl))bis(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4,1-phenylene))bis(methylene))bis(4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium) trifluoroacetate (26 mg, 40% yield) was obtained. HRMS: (M⁺²- H⁺)+=5984.8555, Rt=2.52 min (5 min acidic method).

### Synthesis of 1-(4-((14S,17S)-1-(9H-fluoren-9-yl)-14-isopropyl-8-(2-(((4-nitrophenoxy)carbonyl)oxy)ethyl)-3,7,12,15-tetraoxo-17-(3-ureidopropyl)-2,11-dioxa-4,8,13,16-tetraazaoctadecan-18-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

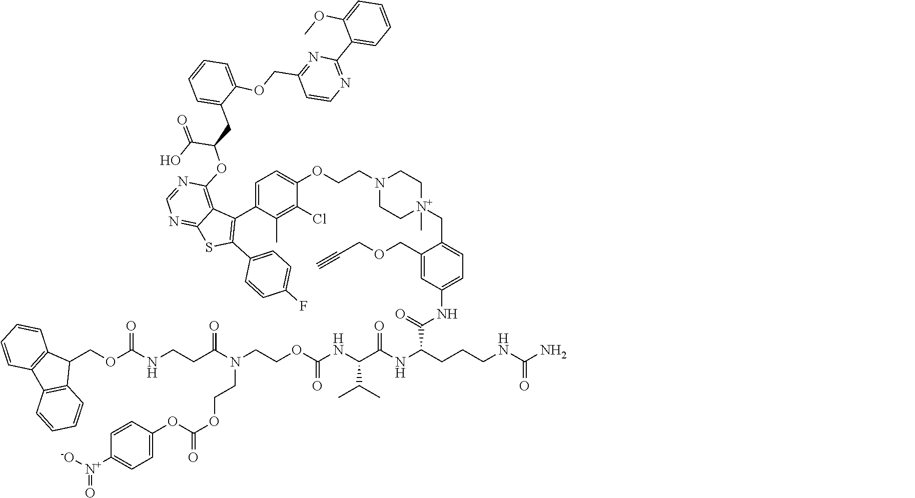

To a solution of 1-(4-((S)-2-((S)-2-amino-3-methylbutanamido)-5-ureidopentanamido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (910 mg, 552 μmol) and (9H-fluoren-9-yl)methyl (3-(bis(2-(((4-nitrophenoxy)carbonyl)oxy)ethyl)amino)-3-oxopropyl)carbamate (2120 mg, 2760 μmol) in DMF (4 mL) was added DIEA (962 μL, 5520 μmol). After stirring for 2 hours, the solution was diluted with DMSO and purified by RP-HPLC. After lyophilization, 1-(4-((14S,17S)-1-(9H-fluoren-9-yl)-14-isopropyl-8-(2-(((4-nitrophenoxy)carbonyl)oxy)ethyl)-3,7,12,15-tetraoxo-17-(3-ureidopropyl)-2,11-dioxa-4,8,13,16-tetraazaoctadecan-18-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (755 mg, 72% yield) was obtained. HRMS: M⁺=1893.7000, Rt=3.04 min (5 min acidic method).

### Synthesis of 1-(4-((6S,9S,21S,24S)-1-amino-15-(3-aminopropanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((prop-2-yn-1-yloxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

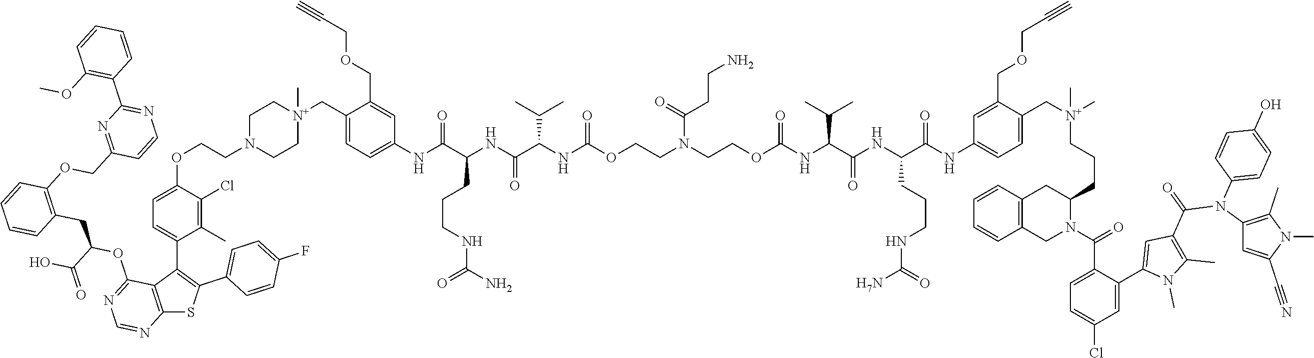

To a solution of 1-(4-((14S,17S)-1-(9H-fluoren-9-yl)-14-isopropyl-8-(2-(((4-nitrophenoxy)carbonyl)oxy)ethyl)-3,7,12,15-tetraoxo-17-(3-ureidopropyl)-2,11-dioxa-4,8,13,16-tetraazaoctadecan-18-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (300 mg, 158 μmol), N-(4-((S)-2-((S)-2-amino-3-methylbutanamido)-5-ureidopentanamido)-2-((prop-2-yn-1-yloxy)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (208 mg, 174 μmol) in DMF (2.1 mL) was added DIEA (276 μL, 1584 μmol). After stirring for 16 hours, 2M Me₂NH in MeOH (792 μL, 1584 μmol). After stirring for 2 hours, the solution was diluted with DMSO and purified by RP-HPLC to yield 1-(4-((6S,9S,21S,24S)-1-amino-15-(3-aminopropanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((prop-2-yn-1-yloxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (320 mg, 76% yield). HRMS: (M⁺²- H⁺)+=2664.7000, Rt=2.38 min (5 min acidic method).

### Synthesis of 1-(4-((6S,9S,21S,24S)-1-amino-15-(3-aminopropanoyl)-6-((3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

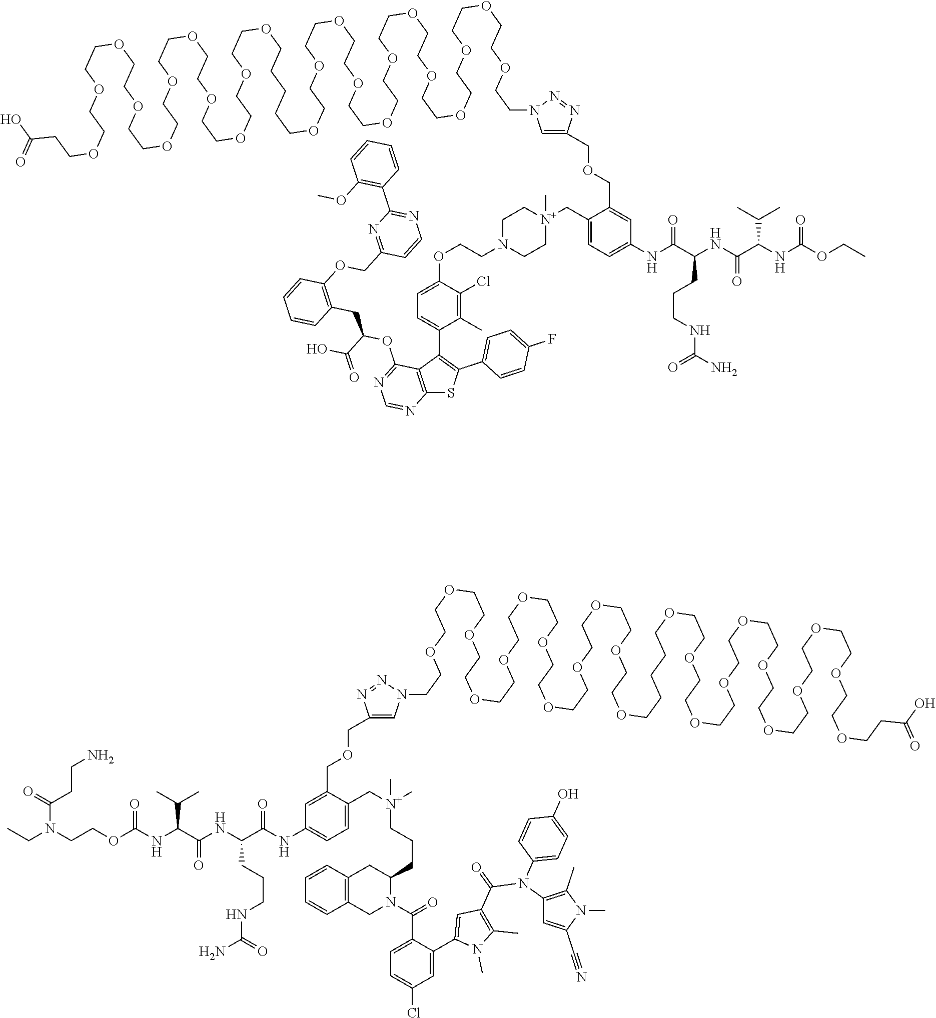

Following the GENERAL PROCEDURE #7 using 1-(4-((6S,9S,21S,24S)-1-amino-15-(3-aminopropanoyl)-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((prop-2-yn-1-yloxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (320 mg, 115 μmol), 1-azido-3,6,9,12,15,18,21,24,27,30,33,36,39,42,45,48,51,54,57,60,63,66,69,72-tetracosaoxapentaheptacontan-75-oic acid (540 mg, 460 μmol), sodium ascorbate 16 mg/mL in H₂O solution (4270 μL, 345 μmol) and CuSO₄ pentahydrate 4 mg/mL H₂O solution (2870 μL, 46 μmol), 1-(4-((6S,9S,21S,24S)-1-amino-15-(3-aminopropanoyl)-6-((3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (474 mg, 80% yield) was obtained. HRMS: (M⁺²- H⁺)+=5007.6201, Rt=2.35 min (5 min acidic method).

### Synthesis of 1-(4-((2S,5S)-11-((6S,9S)-1-amino-6-((3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9-isopropyl-1,8,11-trioxo-12-oxa-2,7,10-triazatetradecan-14-yl)-42-(2,5-dioxo-2,5-dihydro-1H-pyrrol-1-yl)-5-isopropyl-4,7,12,16-tetraoxo-2-(3-ureidopropyl)-8,19,22,25,28,31,34,37,40-nonaoxa-3,6,11,15-tetraazadotetracontanamido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L19-P2)

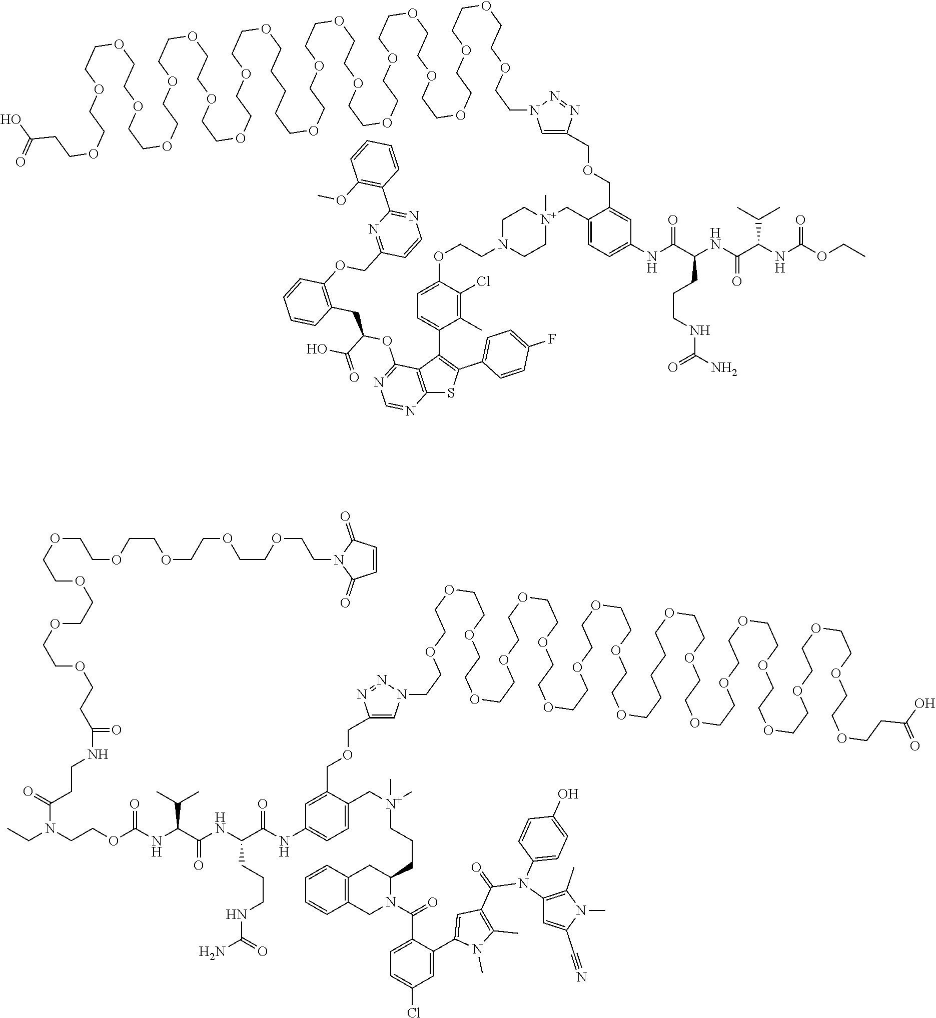

Following the second part of GENERAL PROCEDURE #5 and using 1-(4-((6S,9S,21S,24S)-1-amino-15-(3-aminopropanoyl)-6-((3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (474 mg, 92 μmol), 2,5-dioxopyrrolidin-1-yl 1-(2,5-dioxo-2,5-dihydro-1H-pyrrol-1-yl)-3,6,9,12,15,18,21,24-octaoxaheptacosan-27-oate (114 mg, 185 μmol) and DIEA (161 μL, 925 μmol), 1-(4-((2S,5S)-11-((6S,9S)-1-amino-6-((3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9-isopropyl-1,8,11-trioxo-12-oxa-2,7,10-triazatetradecan-14-yl)-42-(2,5-dioxo-2,5-dihydro-1H-pyrrol-1-yl)-5-isopropyl-4,7,12,16-tetraoxo-2-(3-ureidopropyl)-8,19,22,25,28,31,34,37,40-nonaoxa-3,6,11,15-tetraazadotetracontanamido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (296 mg, 58% yield) was obtained. HRMS: (M⁺²- H⁺)+=5510.8701, Rt=2.49 min (5 min acidic method).

### Synthesis of 1-(4-((6S,9S,21S,24S)-1-amino-15-(3-aminopropanoyl)-6-((4-(((3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)prop-2-yn-1-yl)dimethylammonio)methyl)-3-((prop-2-yn-1-yloxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

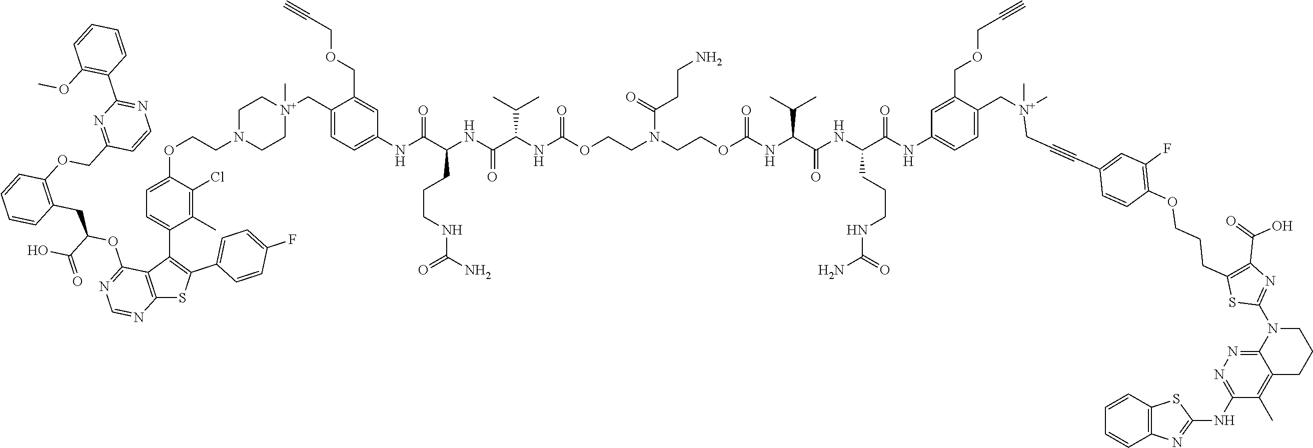 [SMILES: C#CCOCc1cc(NC(=O)[C@H](CCCNC(N)=O)NC(=O)[C@@H](NC(=O)OCCN(CCOC(=O)N[C@H](C(=O)N[C@@H](CCCNC(N)=O)C(=O)Nc2ccc(C[N+]3(C)CCN(CCOc4ccc(-c5c(-c6ccc(F)cc6)sc6ncnc(O[C@H](Cc7ccccc7OCc7ccnc(-c8ccccc8OC)n7)C(=O)O)c56)c(C)c4Cl)CC3)c(COCC#C)c2)C(C)C)C(=O)CCN)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

To a solution of 1-(4-((14S,17S)-1-(9H-fluoren-9-yl)-14-isopropyl-8-(2-(((4-nitrophenoxy)carbonyl)oxy)ethyl)-3,7,12,15-tetraoxo-17-(3-ureidopropyl)-2,11-dioxa-4,8,13,16-tetraazaoctadecan-18-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (1020 mg, 508 μmol), N-(4-((S)-2-((S)-2-amino-3-methylbutanamido)-5-ureidopentanamido)-2-((prop-2-yn-1-yloxy)methyl)benzyl)-3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)-N,N-dimethylprop-2-yn-1-aminium trifluoroacetate (971 mg, 559 μmol) in DMF (6 mL) was added DIEA (656 mg, 508 μmol). After stirring for 16 hours, 2M Me₂NH in THF (2.54 mL, 5080 μmol). After stirring for 2 hours, the solution was diluted with DMSO and purified by RP-HPLC to yield 1-(4-((6S,9S,21S,24S)-1-amino-15-(3-aminopropanoyl)-6-((4-(((3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)prop-2-yn-1-yl)dimethylammonio)methyl)-3-((prop-2-yn-1-yloxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (780 mg, 52% yield). HRMS: (M⁺²- H⁺)+=2619.0100, Rt=2.39 min (5 min acidic method).

### Synthesis of 1-(4-((6S,9S,21S,24S)-1-amino-15-(3-aminopropanoyl)-6-((4-(((3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)prop-2-yn-1-yl)dimethylammonio)methyl)-3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

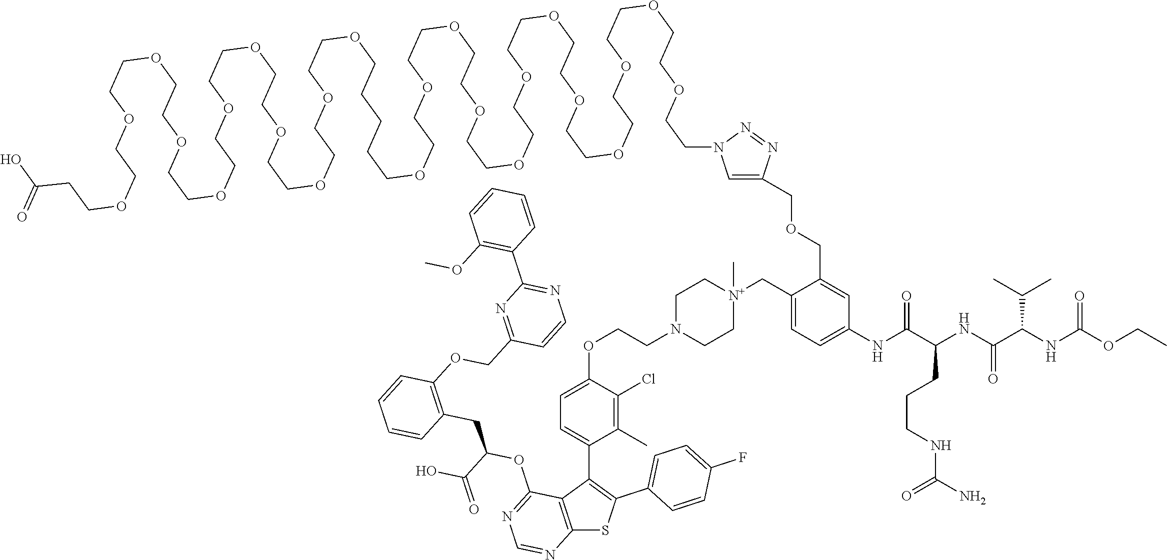 [SMILES: CCOC(=O)N[C@H](C(=O)N[C@@H](CCCNC(N)=O)C(=O)Nc1ccc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)c(COCc2cn(CCOCCOCCOCCOCCOCCOCCOCCOCCOCCOCCOCCOCCCCCOCCOCCOCCOCCOCCOCCOCCOCCOCCOCCOCCC(=O)O)nn2)c1)C(C)C]

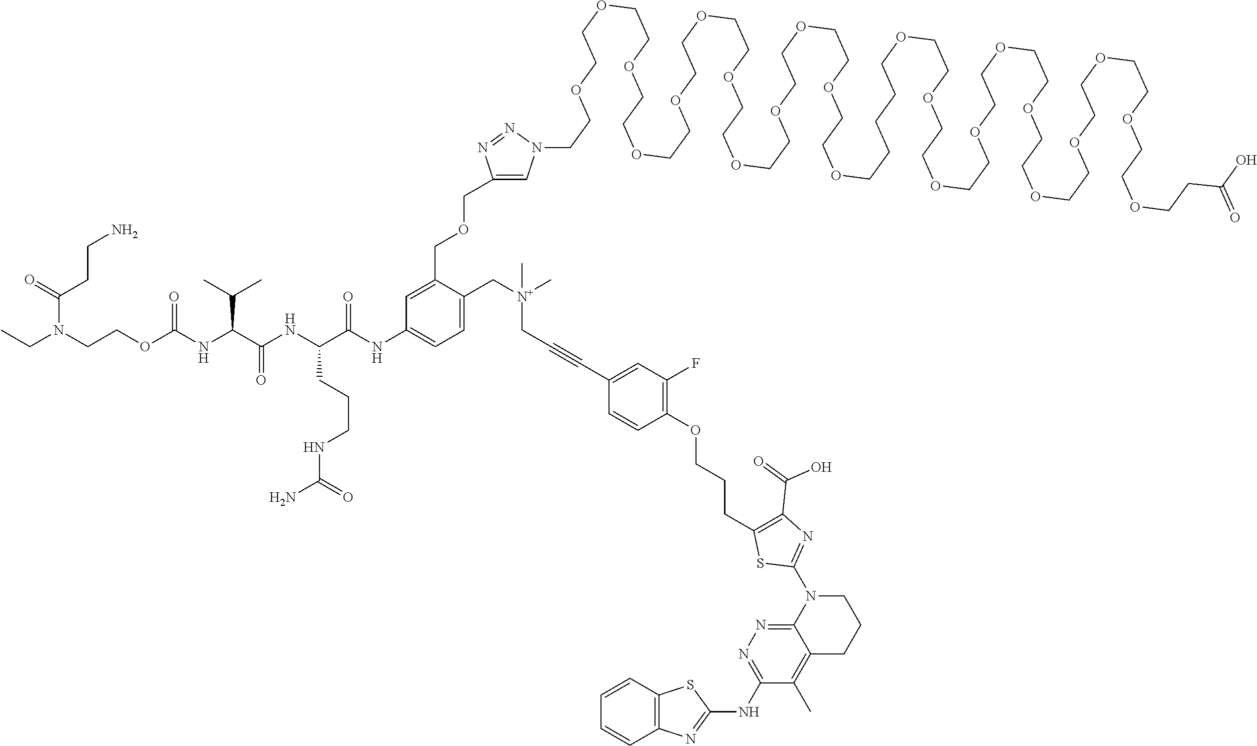 [SMILES: CCN(CCOC(=O)N[C@H](C(=O)N[C@@H](CCCNC(N)=O)C(=O)Nc1ccc(C[N+](C)(C)CC#Cc2ccc(OCCCc3sc(N4CCCc5c4nnc(Nc4nc6ccccc6s4)c5C)nc3C(=O)O)c(F)c2)c(COCc2cn(CCOCCOCCOCCOCCOCCOCCOCCOCCOCCOCCOCCOCCCCCOCCOCCOCCOCCOCCOCCOCCOCCOCCOCCOCCC(=O)O)nn2)c1)C(C)C)C(=O)CCN]

Following the GENERAL PROCEDURE #7 using 1-(4-((6S,9S,21S,24S)-1-amino-15-(3-aminopropanoyl)-6-((4-(((3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)prop-2-yn-1-yl)dimethylammonio)methyl)-3-((prop-2-yn-1-yloxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-((prop-2-yn-1-yloxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (678 mg, 229 μmol), 1-azido-3,6,9,12,15,18,21,24,27,30,33,36,39,42,45,48,51,54,57,60,63,66,69,72-tetracosaoxapentaheptacontan-75-oic acid (1073 mg, 915 μmol), sodium ascorbate 48 mg/mL in H₂O solution (2833 μL, 686 μmol) and CuSO₄ pentahydrate 12 mg/mL H₂O solution (2858 μL, 137 μmol), 1-(4-((6S,9S,21S,24S)-1-amino-15-(3-aminopropanoyl)-6-((4-(((3-(4-(3-(2-(3-(benzo[ld]thiazol-2-ylamino)-4-methyl-6,7-dihydropyrido[l2,3-c]pyridazin-8(5H)-yl)-4-carboxythiazol-5-yl)propoxy)-3-fluorophenyl)prop-2-yn-1-yl)dimethylammonio)methyl)-3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (839 mg, 69% yield) was obtained. HRMS: (M⁺²- H⁺)⁺=4962.3223, Rt=2.37 min (5 min acidic method).

### Synthesis of 1-(4-((2S,5S)-11-((6S,9S)-1-amino-6-((4-(((3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)prop-2-yn-1-yl)dimethylammonio)methyl)-3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)phenyl)carbamoyl)-9-isopropyl-1,8,11-trioxo-12-oxa-2,7,10-triazatetradecan-14-yl)-42-(2,5-dioxo-2,5-dihydro-1H-pyrrol-1-yl)-5-isopropyl-4,7,12,16-tetraoxo-2-(3-ureidopropyl)-8,19,22,25,28,31,34,37,40-nonaoxa-3,6,11,15-tetraazadotetracontanamido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L19-P6)

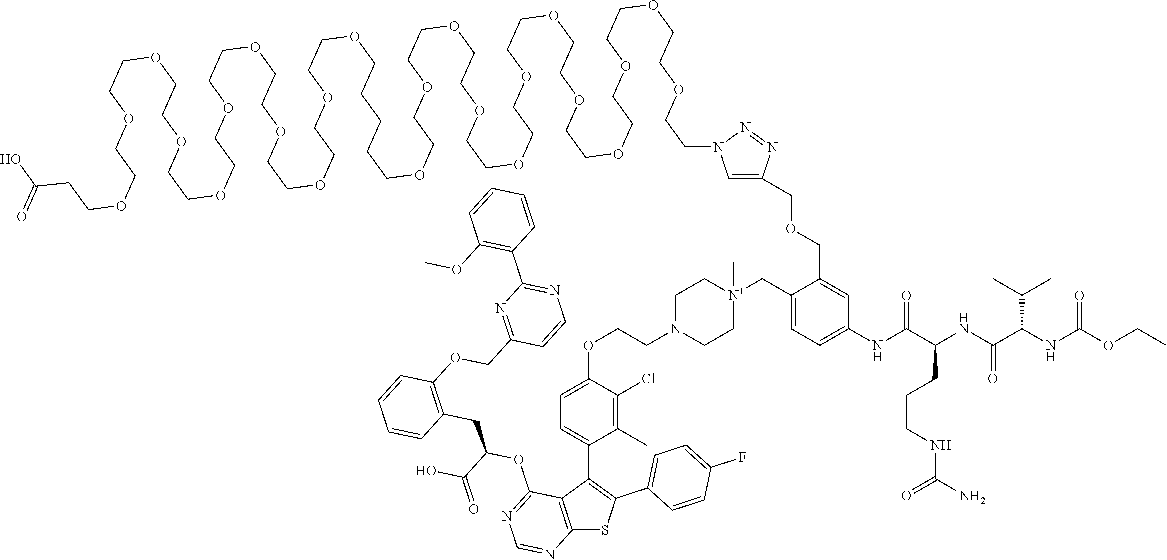 [SMILES: CCOC(=O)N[C@H](C(=O)N[C@@H](CCCNC(N)=O)C(=O)Nc1ccc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)c(COCc2cn(CCOCCOCCOCCOCCOCCOCCOCCOCCOCCOCCOCCOCCCCCOCCOCCOCCOCCOCCOCCOCCOCCOCCOCCOCCC(=O)O)nn2)c1)C(C)C]

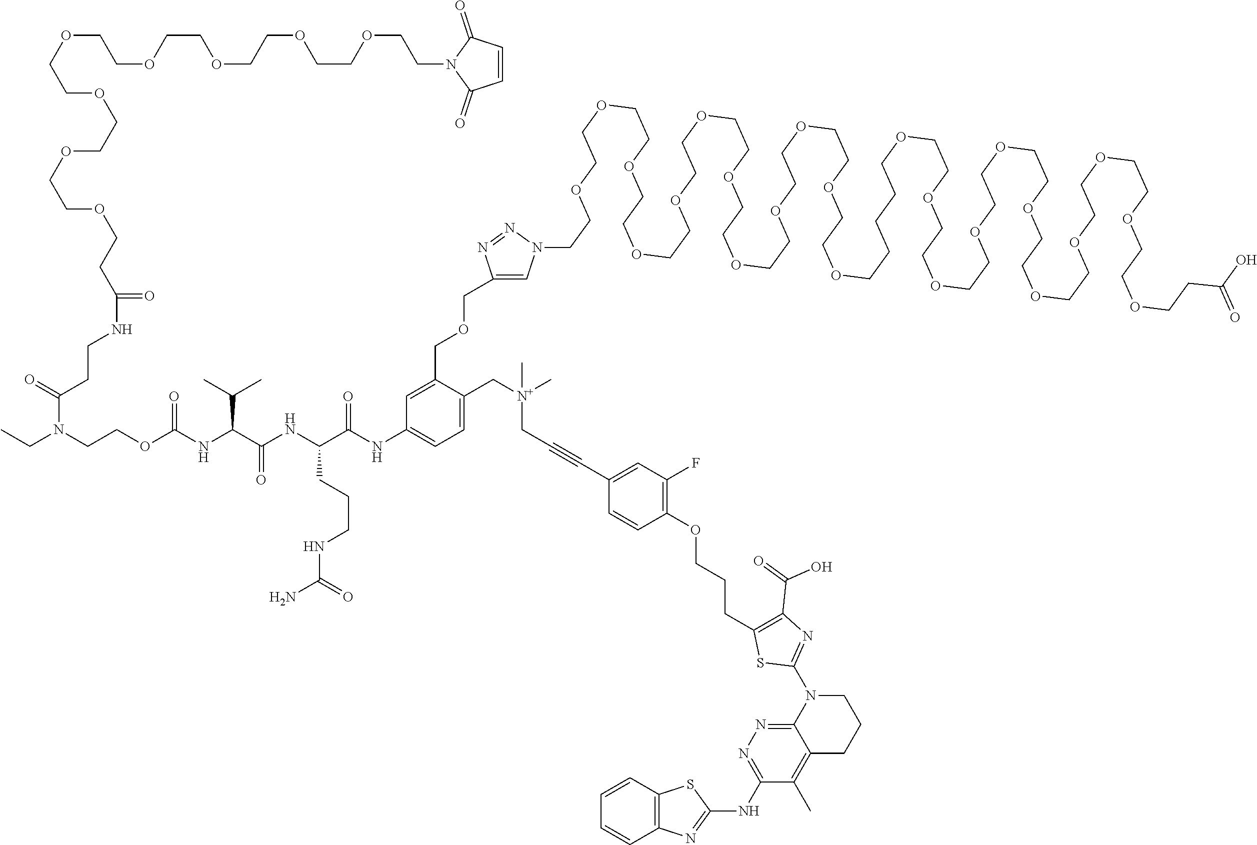 [SMILES: CCN(CCOC(=O)N[C@H](C(=O)N[C@@H](CCCNC(N)=O)C(=O)Nc1ccc(C[N+](C)(C)CC#Cc2ccc(OCCCc3sc(N4CCCc5c4nnc(Nc4nc6ccccc6s4)c5C)nc3C(=O)O)c(F)c2)c(COCc2cn(CCOCCOCCOCCOCCOCCOCCOCCOCCOCCOCCOCCOCCCCCOCCOCCOCCOCCOCCOCCOCCOCCOCCOCCOCCC(=O)O)nn2)c1)C(C)C)C(=O)CCNC(=O)CCOCCOCCOCCOCCOCCOCCOCCOCCN1C(=O)C=CC1=O]

Following the second part of GENERAL PROCEDURE #5 and using 1-(4-((6S,9S,21S,24S)-1-amino-i5-(3-aminopropanoyl)-6-((4-(((3-(4-(3-(2-(3-(benzo[ld]thiazol-2-ylamino)-4-methyl-6,7-dihydropyrido[2,3-c]pyridazin-8(5H)-yl)-4-carboxythiazol-5-yl)propoxy)-3-fluorophenyl)prop-2-yn-1-yl)dimethylammonio)methyl)-3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)phenyl)carbamoyl)-9,21-diisopropyl-1,8,11,19,22-pentaoxo-24-(3-ureidopropyl)-12,18-dioxa-2,7,10,15,20,23-hexaazapentacosan-25-amido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (988 mg, 186 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (230 mg, 372 μmol) and DIEA (241 mg, 1861 μmol), 1-(4-((2S,5S)-11-((6S,9S)-1-amino-6-((4-(((3-(4-(3-(2-(3-(benzo[d]thiazol-2-ylamino)-4-methyl-6,7-dihydropyrido[2,3-c]pyridazin-8(5H)-yl)-4-carboxythiazol-5-yl)propoxy)-3-fluorophenyl)prop-2-yn-1-yl)dimethylammonio)methyl)-3-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)phenyl)carbamoyl)-9-isopropyl-1,8,11-trioxo-12-oxa-2,7,10-triazatetradecan-14-yl)-42-(2,5-dioxo-2,5-dihydro-1H-pyrrol-l-yl)-5-isopropyl-4,7,12,16-tetraoxo-2-(3-ureidopropyl)-8,19,22,25,28,31,34,37,40-nonaoxa-3,6,11,15-tetraazadotetracontanamido)-2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (821 mg, 77% yield) was obtained. HRMS: (M⁺²- H⁺)+=5465.5601, Rt=2.49 min (5 min acidic method).

### Synthesis of 4-[2-(4-{4-[(1R)-1-Carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-{[4-{(2S)-2-[(2S)-2-{[(2-{4-[(5S)-5-{[(2S)-1-({(2S)-1-[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)anilino]-1-oxopropan-2-yl}amino)-3-methyl-1-oxobutan-2-yl]carbamoyl}-37-(2,5-dioxo-2,5-dihydro-1H-pyrrol-1-yl)-3,11-dioxo-2,14,17,20,23,26,29,32,35-nonaoxa-4,10-diazaheptatriacontan-1-yl]-1H-1,2,3-triazol-1-yl}ethoxy)carbonyl]amino}-3-methylbutanamido]propanamido}-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]methyl}-1-methylpiperazin-1-ium trifluoroacetate (P1-L25-P1)

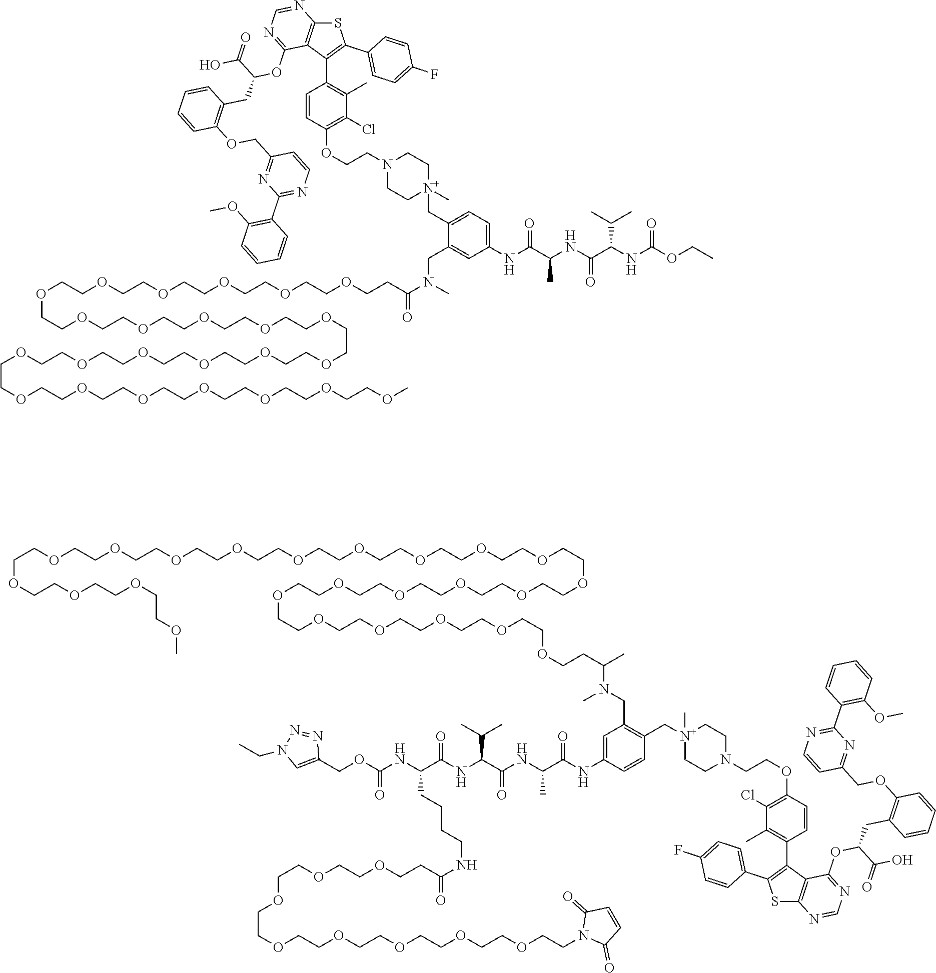

Following the GENERAL PROCEDURE #7 using 1-(4-((S)-2-((S)-2-(((2-azidoethoxy)carbonyl)amino)-3-methylbutanamido)propanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (28 mg, 11 μmol), 4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-(4-((1OS,13S,16S)-13-isopropyl-2,2,16-trimethyl-4,11,14-trioxo-10-(((prop-2-yn-1-yloxy)carbonyl)amino)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (30 mg, 10.6 μmol), sodium ascorbate 32 mg/mL in H₂O solution (327 μL, 53 μmol) and CuSO₄ pentahydrate 8 mg/mL H₂O solution (330 μL, 10.6 μmol), click product (22.6 mg, 40% yield) was obtained. HRMS: (M⁺²-H⁺)⁺=5126.4302, Rt=2.81 min (5 min acidic method). Following the GENERAL PROCEDURE #5 using click product (26 mg, 4.4 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (7.8 mg, 13 μmol) and DIEA (10.9 p L, 63 μmol), 4-[2-(4-{4-[(1R)-1-Carboxy-2-(2-{1[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1- {[4-{(2S)-2-[(2S)-2-{[(2-{4-[(5S)-5-{[(2S)-1-({(2S)-1-[4-({4-[2-(4-{4-[(1iR)-1-carboxy-2-(2-{[2-(2- methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-l-yl}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)anilino]-1-oxopropan-2-yl}amino)-3-methyl-1-oxobutan-2-yl]carbamoyl}-37-(2,5-dioxo-2,5-dihydro-1H-pyrrol-1-yl)-3,11-dioxo-2,14,17,20,23,26,29,32,35-nonaoxa-4,10-diazaheptatriacontan-l-yl]-1H-1,2,3-triazol-l-yl}ethoxy)carbonyl]amino)-3-methylbutanamido]propanamido}-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]methyl}-1-methylpiperazin-1-ium trifluoroacetate (20.9 mg, 80% yield) was obtained. HRMS: (M⁺²- H⁺)+=5409.5898, Rt=2.66 min (5 min acidic method).

### Synthesis of 1-{[4-{[(33S,36S,39S)-33-{[({1-[(9S,12S)-17-Amino-12-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-4,7,10,17-tetraoxo-9-(propan-2-yl)-3-oxa-5,8,11,16-tetraazaheptadecan-1-yl]-1H-1,2,3-triazol-4-yl}methoxy)carbonyl]amino}-39-(3-carbamamidopropyl)-1-(2,5-dioxo-2,5-dihydro-1H-pyrrol-1-yl)-27,34,37,40-tetraoxo-36-(propan-2-yl)-3,6,9,12,15,18,21,24-octaoxa-28,35,38-triazatetracontan-40-yl]amino}-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]methyl}-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (P1-L26-P1)

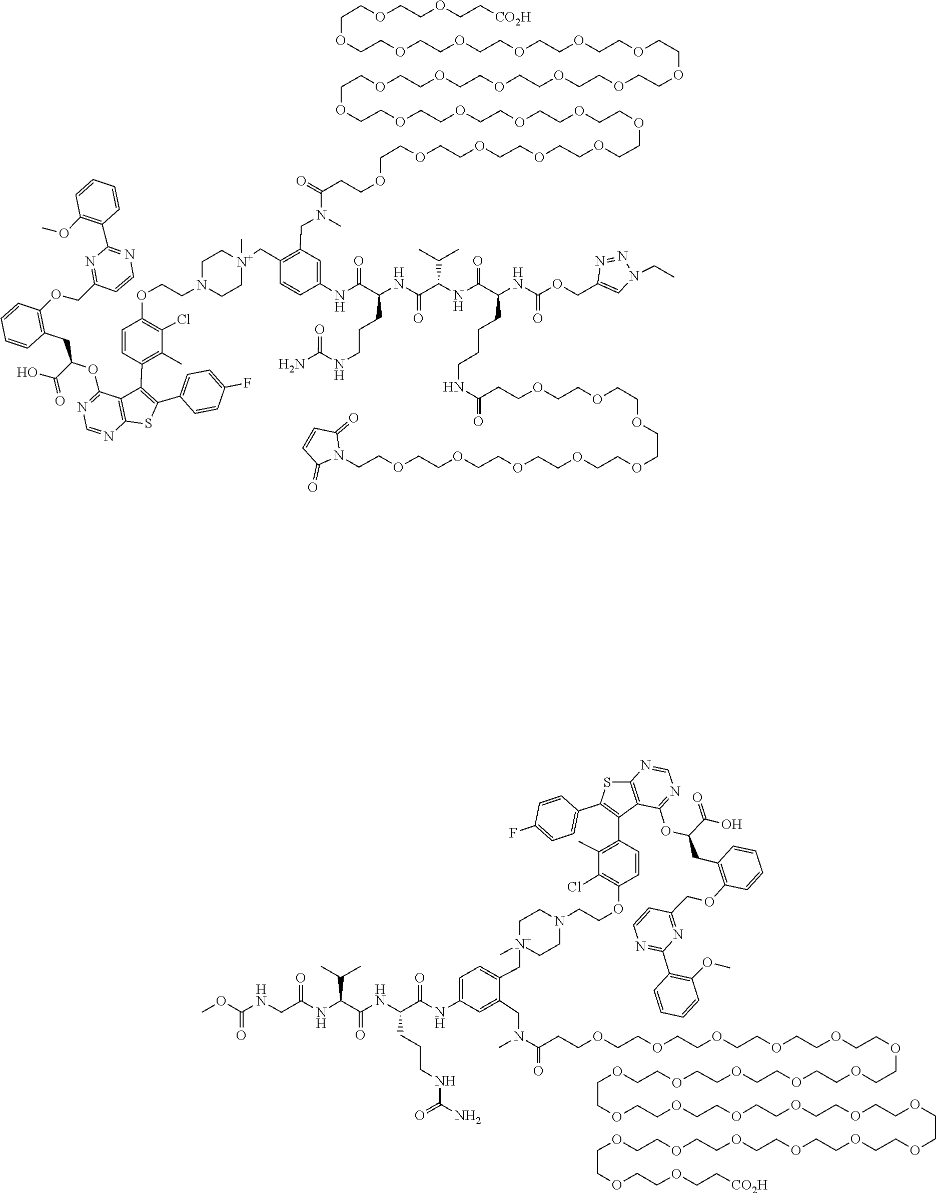

Following the GENERAL PROCEDURE #7 using 1-(2-(((((9H-fluoren-9-yl)methoxy)carbonyl)(methyl)amino)methyl)-4-((1oS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (44.2 mg, 21 μmol), 1-(4-((9S,12S)-1-azido-9-isopropyl-4,7,10-trioxo-12-(3-ureidopropyl)-3-oxa-5,8,11-triazatridecan-13-amido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (37.5 mg, 21 μmol), sodium ascorbate 16 mg/mL in H₂O solution (777 μL, 63 μmol) and CuSO₄ pentahydrate 4 mg/mL H₂O solution (653 μL, 10.5 μmol), the click product (26.8 mg, 39% yield) was obtained. HRMS: ((M⁺²- H⁺)+=3260.3000, Rt=2.76 min (5 min acidic method). The obtained click product (26.8 mg, 8.2 μmol) was dissolved in DMSO (2 mL) and was treated with 2M Me₂NH in MeOH (82 μL, 164 μmol). After stirring for 30 minutes, the solution was purified by RP-HPLC to yield after lyophilization FMOC deprotected click product (21.4 mg, 86% yield). HRMS: ((M⁺²- H⁺)+=3038.2500, Rt=2.41 min (5 min acidic method). To a solution of FMOC deprotected click product (21.4 mg, 6.3 μmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (25 mg, 19 μmol) in DMF (1 mL) was added DIEA (8.8 μL, 51 μmol). After stirring for 16 hours, the solution was diluted with DMSO and purified by RP-HPLC. After lyophilization, pegylated click product (15.3 mg, 42% yield) was obtained. HRMS: (M⁺²- H⁺)+=5439.5498, Rt=2.69 min (5 min acidic method). Following the GENERAL PROCEDURE #5 using pegylated click product (15.3 mg, 2.6 μmol), 2,5-dioxopyrrolidin-1-yl 1-(2,5-dioxo-2,5-dihydro-1H-pyrrol-1-yl)-3,6,9,12,15,18,21,24-octaoxaheptacosan-27-oate (4.9 mg, 7.9 μmol) and DIEA (9.2 μL, 53 μmol), 1-{[4-{[(33S,36S,39S)-33-{[({1-[(9S,12S)-17-Amino-12-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-4,7,10,17-tetraoxo-9-(propan-2-yl)-3-oxa-5,8,11,16-tetraazaheptadecan-1-yl]-1H-1,2,3-triazol-4-yl}methoxy)carbonyl]amino}-39-(3-carbamamidopropyl)-1-(2,5-dioxo-2,5-dihydro-1H-pyrrol-1-yl)-27,34,37,40-tetraoxo-36-(propan-2-yl)-3,6,9,12,15,18,21,24-octaoxa-28,35,38-triazatetracontan-40-yl]amino}-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]methyl}-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (8.4 mg, 51% yield) was obtained. HRMS: (M⁺²- H⁺)+=5842.7300, Rt=2.74 min (5 min acidic method).

### Synthesis of 1-(3-(3-((S)-6-((((9H-fluoren-9-yl)methoxy)carbonyl)amino)-2-((tert-butoxycarbonyl)amino)hexanamido)propanamido)-4-(((2S,3R,4S,5S,6S)-6-((allyloxy)carbonyl)-3,4,5-tris(((allyloxy)carbonyl)oxy)tetrahydro-2H-pyran-2-yl)oxy)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

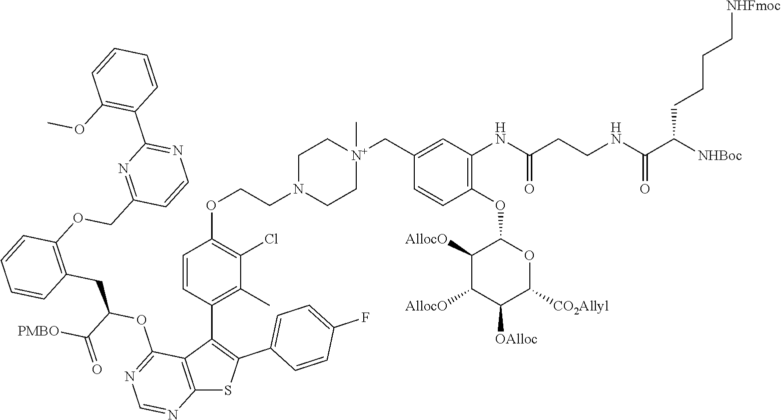

A mixture of 4-methoxybenzyl (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoate (100 mg, 0.100 mmol), allyl (2S,3S,4S,5R,6S)-6-(2-(3-((S)-6-((((9H-fluoren-9-yl)methoxy)carbonyl)amino)-2-((tert-butoxycarbonyl)amino)hexanamido)propanamido)-4-(chloromethyl)phenoxy)-3,4,5-tris(((allyloxy)carbonyl)oxy)tetrahydro-2H-pyran-2-carboxylate (250 mg, 0.218 mmol), and tetrabutylammonium iodide (15 mg, 0.041 mmol) in DMSO (0.8 mL) was stirred at RT for 17 h. The mixture was loaded onto RP-ISCO column (50g, gold). The column was eluted with MeCN-water (0.1% TFA modifier) to give the title product with minor impurity present as a cream-color solid (302 mg). HRMS: M⁺=2106.6699, Rt=3.19 min (5 min acidic method). This product was used in the next step without further purification.

### Synthesis of 1-(3-(3-((S)-6-amino-2-((tert-butoxycarbonyl)amino)hexanamido)propanamido)-4-(((2S,3R,4S,5S,6S)-6-carboxy-3,4,5-trihydroxytetrahydro-2H-pyran-2-yl)oxy)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

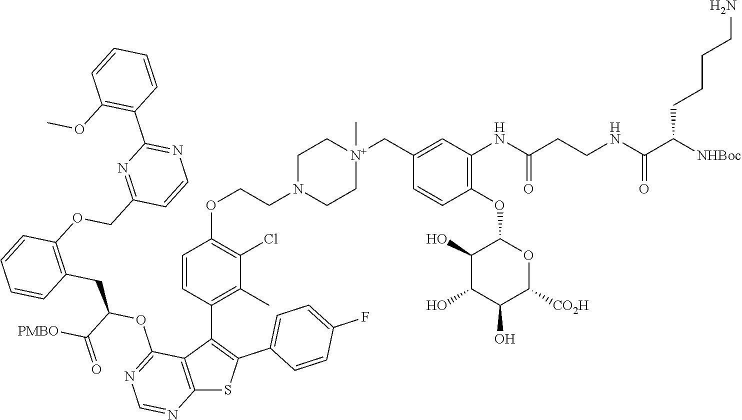

A mixture of 1-(3-(3-((S)-6-((((9H-fluoren-9-yl)methoxy)carbonyl)amino)-2-((tert-butoxycarbonyl)amino)hexanamido)propanamido)-4-(((2S,3R,4S,5S,6S)-6-((allyloxy)carbonyl)-3,4,5-tris(((allyloxy)carbonyl)oxy)tetrahydro-2H-pyran-2-yl)oxy)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium (302 mg, obtained in the previous step), Pd(Ph₃P)₄ (16 mg, 0.014 mmol), formic acid (20 μl, 0.52 mmol), and TEA (96 μl, 0.69 mmol) in THF (3 mL) was stirred at RT for 50 min. To this was then added dimethylamine in THF (2M, 1.4 mL, 2.80 mmol). The mixture was stirred at RT for 1 h. The mixture was diluted with DMSO (4 mL) and loaded onto RP-ISCO column (150 g, gold). The column was eluted with MeCN-water (0.1% TFA modifier). Fractions containing the desired product were combined and lyophilized to give the title product as a white solid with minor impurity present (140 mg). This product was used in the next step without further purification. HRMS: M⁺=1952.5600, Rt=2.38 min (5 min acidic method).

### Synthesis of 1-(3-((S)-82-((tert-butoxycarbonyl)amino)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

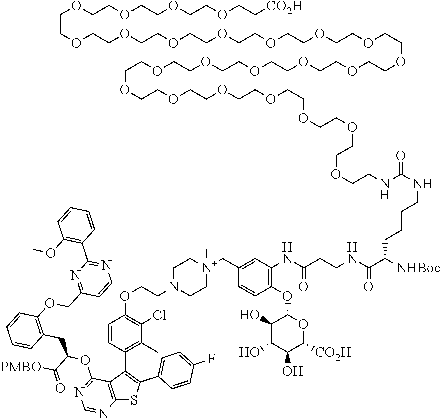

A mixture of 1-amino-3,6,9,12,15,18,21,24,27,30,33,36,39,42,45,48,51,54,57,60,63,66,69,72-tetracosaoxapentaheptacontan-75-oic acid (132 mg, 0.115 mmol), bis(4-nitrophenyl) carbonate (35 mg, 0.12 mmol), and DIPEA (34 p L, 0.19 mmol) in DMF (1 mL) was stirred at RT for 5 min. LCMS 1 showed formation of PEG carbamate and consumption of bis-p-nitrophenyl carbonate. To this was added 1-(3-(3-((S)-6-amino-2-((tert-butoxycarbonyl)amino)hexanamido)propanamido)-4-(((2S,3R,4S,5S,6S)-6-carboxy-3,4,5-trihydroxytetrahydro-2H-pyran-2-yl)oxy)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium (140 mg, obtained from previous step) and DIPEA (100 μL, 0.573 mmol). The mixture was stirred at RT for 70 min. The mixture was diluted with DMSO (5 mL) and loaded onto RP-ISCO column (150g, gold). The column was eluted with MeCN-water (0.1% TFA modifier). Fractions containing the desired product were combined and lyophilized to give the title product as a white solid (TFA salt, 130 mg, 45% yield over the last three steps). HRMS: M⁺=2763.29, Rt=2.72 min (5 min acidic method).

### Synthesis of 1-(3-((S)-82-amino-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

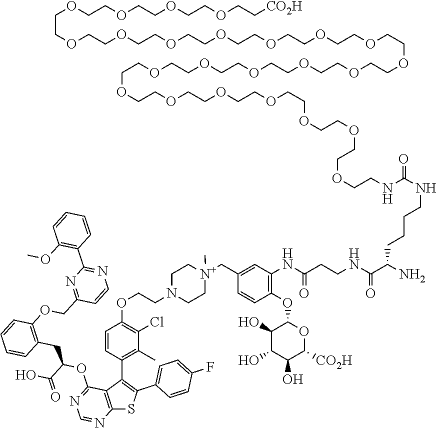 [SMILES: COc1ccccc1-c1nccc(COc2ccccc2C[C@@H](Oc2ncnc3sc(-c4ccc(F)cc4)c(-c4ccc(OCCN5CC[N+](C)(Cc6ccc(O[C@@H]7O[C@H](C(=O)O)[C@@H](O)[C@H](O)[C@H]7O)c(NC(=O)CCNC(=O)[C@@H](N)CCCCNC(=O)NCCOCCOCCOCCOCCOCCOCCOCCOCCOCCOCCOCCOCCOCCOCCOCCOCCOCCOCCOCCOCCOCCOCCOCCOCCC(=O)O)c6)CC5)c(Cl)c4C)c23)C(=O)O)n1]

A mixture of 1-(3-((S)-82-((tert-butoxycarbonyl)amino)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium (TFA salt, 130 mg, 0.0450 mmol) and TFA (2.0 μl, 0.026 mmol) in DCM (5 mL) was stirred at RT for 20 min. The mixture was concentrated. The residue was taken up in DCM and concentrated. The residue was then taken up in MeCN and concentrated. Ether was added to the residual oil, inducing sticky solid formation, which was sonicated. The ether layer was removed, and the residue was dried under vacuum to give the title product as a white solid (bis TFA salt, 103 mg, quantitative yield). HRMS: M⁺=2543.1399, Rt=2.14 min (5 min acidic method).

### Synthesis of 1-(3-((S)-82-((S)-6-((tert-butoxycarbonyl)amino)-2-(((prop-2-yn-1-yloxy)carbonyl)amino)hexanamido)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

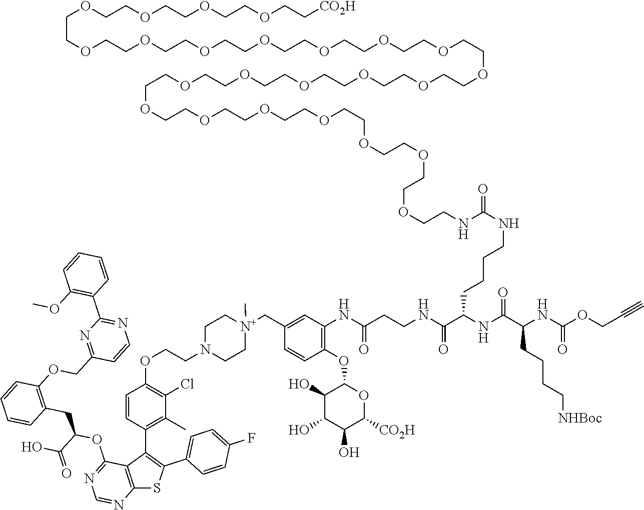 [SMILES: C#CCOC(=O)N[C@@H](CCCCNC(=O)OC(C)(C)C)C(=O)N[C@@H](CCCCNC(=O)NCCOCCOCCOCCOCCOCCOCCOCCOCCOCCOCCOCCOCCOCCOCCOCCOCCOCCOCCOCCOCCOCCOCCOCCOCCC(=O)O)C(=O)NCCC(=O)Nc1cc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)ccc1O[C@@H]1O[C@H](C(=O)O)[C@@H](O)[C@H](O)[C@H]1O]

A mixture of N6-(tert-butoxycarbonyl)-N2-((prop-2-yn-1-yloxy)carbonyl)-L-lysine (22 mg, 0.067 mmol), HBTU (16 mg, 0.042 mmol), and DIPEA (0.024 mL, 0.14 mmol) in DMF (0.7 mL) was stirred at RT for 15 min, and the solution was added to 1-(3-((S)-82-amino-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium (bis TFA salt, 38 mg, 0.014 mmol). The mixture was stirred at RT for 10 min. The mixture was loaded to RP-ISCO column (150g, gold), and the column was eluted with MeCN-water (0.1% TFA modifier). Fractions containing the desired product were combined and lyophilized to give the title product as a white solid, which was used in the next step without further purification. HRMS: M⁺=2853.33, Rt=2.69 min (5 min acidic method).

### Synthesis of 1-(3-((S)-82-((S)-6-amino-2-(((prop-2-yn-1-yloxy)carbonyl)amino)hexanamido)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

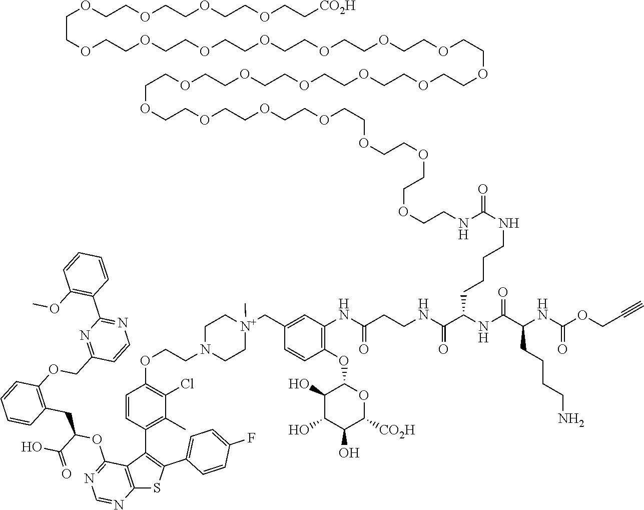 [SMILES: C#CCOC(=O)N[C@@H](CCCCN)C(=O)N[C@@H](CCCCNC(=O)NCCOCCOCCOCCOCCOCCOCCOCCOCCOCCOCCOCCOCCOCCOCCOCCOCCOCCOCCOCCOCCOCCOCCOCCOCCC(=O)O)C(=O)NCCC(=O)Nc1cc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)ccc1O[C@@H]1O[C@H](C(=O)O)[C@@H](O)[C@H](O)[C@H]1O]

A mixture of 1-(3-((S)-82-((S)-6-((tert-butoxycarbonyl)amino)-2-(((prop-2-yn-1-yloxy)carbonyl)amino)hexanamido)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium (TFA salt, obtained in the previous step) and TFA (1.0 mL, 13 mmol) was stirred at RT for 30 min. The mixture was concentrated. The residue was then taken up in MeCN and concentrated. Ether was added to the residual oil to induce precipitation. The mixture was sonicated, and ether was removed. The resulting solid was taken up in MeCN-water and lyophilized to give the title product as a white solid (bis TFA salt, 12 mg, 30% yield over the last two steps). HRMS: [M⁺+H⁺]⁺ 2/2=1378.09, Rt=2.06 min (5 min acidic method).

### Synthesis of 1-(4-((9S,12S)-1-azido-9-isopropyl-4,7,10-trioxo-12-(3-ureidopropyl)-3-oxa-5,8,11-triazatridecan-13-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

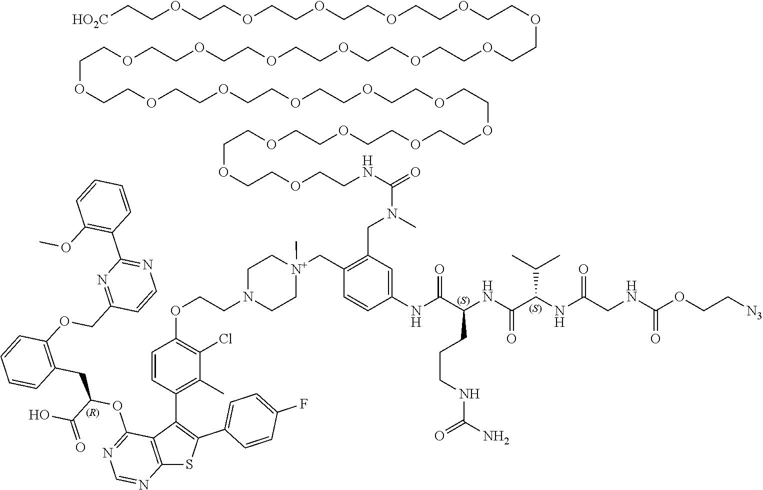

A solution of ((2-azidoethoxy)carbonyl)glycine (22.2 mg, 0.106 mmol), HATU (33.7 mg, 0.089 mmol) and DIPEA (0.031 mL, 0.177 mmol) in 0.5 ml of DMF was stirred at RT for 15 min. The resulted solution was added into another solution of 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (100 mg, 0.035 mmol) and DIPEA (0.031 mL, 0.177 mmol) in DMF (0.5 mL). The reaction mixture was stirred at RT for 20 min, diluted with DMSO, and purified by RP-ISCO. After lyophilization, the title product (34 mg, 0.012 mmol, 33% yield) was obtained. HRMS: M⁺=2621.2200, Rt=2.33 min (5 min acidic method).

### Synthesis of 2-{[(82S)-82-{[(33S)-33-{[({1-[(9S,12S)-17-Amino-12-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontan-1-yl)phenyl]carbamoyl}-4,7,10,17-tetraoxo-9-(propan-2-yl)-3-oxa-5,8,11,16-tetraazaheptadecan-1-yl]-1H-1,2,3-triazol-4-yl}methoxy)carbonyl]amino}-1-(2,5-dioxo-2,5-dihydro-1H-pyrrol-1-yl)-27,34-dioxo-3,6,9,12,15,18,21,24-octaoxa-28-azatetratriacontan-34-yl]amino}-1-carboxy-76,83,87-trioxo-3,6,9,12,15,18,21,24,27,30,33,36,39,42,45,48,51,54,57,60,63,66,69,72-tetracosaoxa-75,77,84-triazaheptaoctacontan-87-yl]amino}-4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)phenyl p-D-glucopyranosiduronic acid (P1-L28-P1)

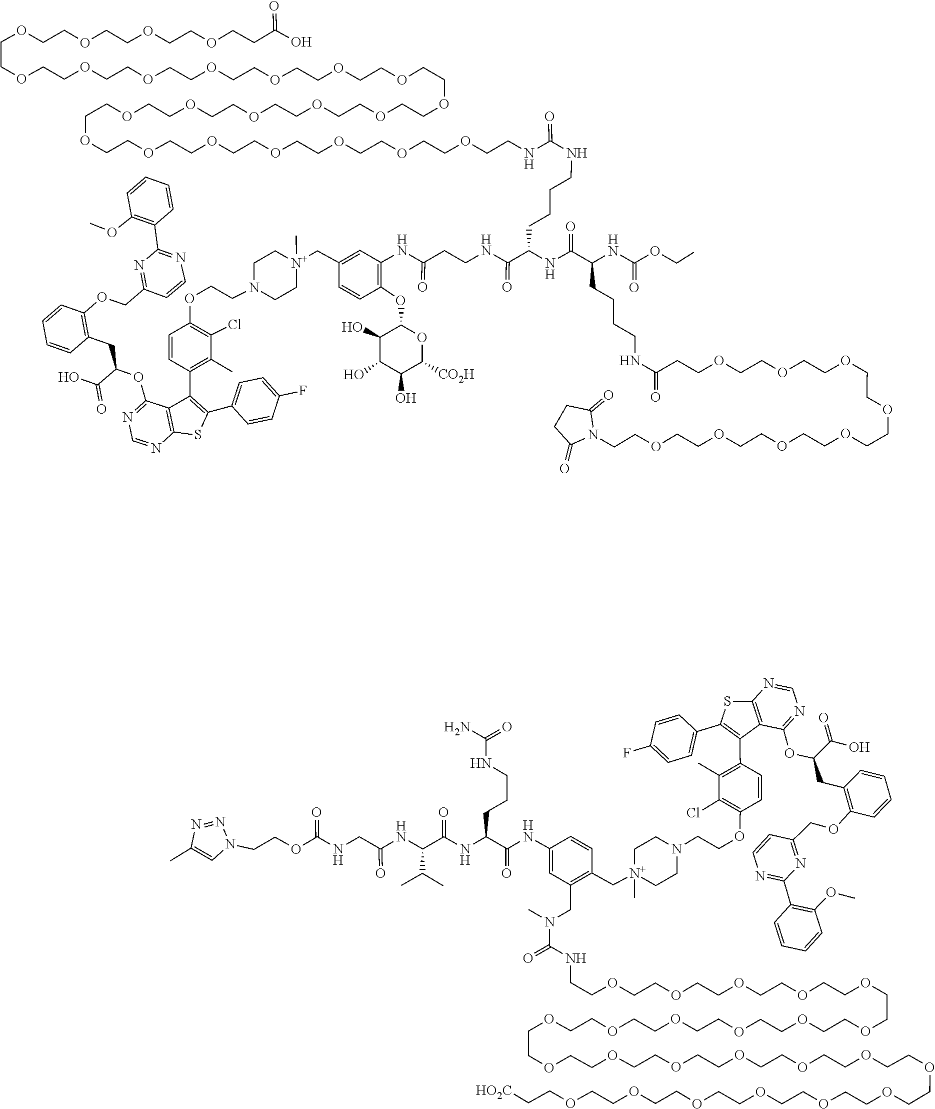

Following the GENERAL PROCEDURE #7 using 1-(3-((S)-82-((S)-6-amino-2-(((prop-2-yn-1-yloxy)carbonyl)amino)hexanamido)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium (12 mg, 4.02 μmol) and 1-(4-((9S,12S)-1-azido-9-isopropyl-4,7,10-trioxo-12-(3-ureidopropyl)-3-oxa-5,8,11-triazatridecan-13-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium (II mg, 4.02 μmol), sodium ascorbate 16 mg/mL in H₂O solution (75 μL, 6.1 μmol) and CuSO₄ pentahydrate 4 mg/mL H₂O solution (75 μL, 1.2 μmol), click product (14 mg, 65% yield) was obtained. HRMS: (M⁺²- H⁺)⁺=5373.4902, Rt=2.84 (5 min acidic method). Following the second part of GENERAL PROCEDURE #5 using isolated click product (14 mg, 2.4 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (8 mg, 13 μmol) and DIEA (5 μL, 29 μmol), 2-{[(82S)-82-{I[(33S)-33-{[(I1-[(9S,12S)-17-Amino-i2-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontan-1-yl)phenyl]carbamoyl}-4,7,10,17-tetraoxo-9-(propan-2-yl)-3-oxa-5,8,11,16-tetraazaheptadecan-1-yl]-1H-1,2,3-triazol-4-yl}methoxy)carbonyl]amino}-1-(2,5-dioxo-2,5-dihydro-1H-pyrrol-1-yl)-27,34-dioxo-3,6,9,12,15,18,21,24-octaoxa-28-azatetratriacontan-34-yl]amino}-1-carboxy-76,83,87-trioxo-3,6,9,12,15,18,21,24,27,30,33,36,39,42,45,48,51,54,57,60,63,66,69,72-tetracosaoxa-75,77,84-triazaheptaoctacontan-87-yl]amino}-4-(4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)phenyl R-D-glucopyranosiduronic acid as a white solid (8 mg, 43% yield). HRMS: (M⁺²- H⁺)⁺=5876.7598, Rt=2.49 (5 min acidic method).

### Synthesis of 1-(3-((S)-82-(((2-azidoethoxy)carbonyl)amino)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

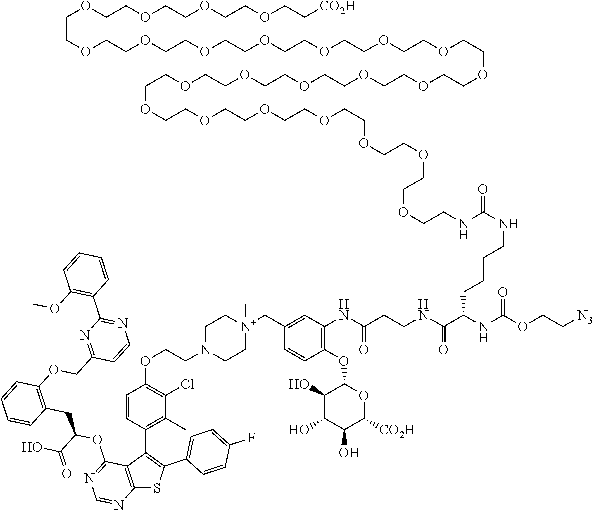 [SMILES: COc1ccccc1-c1nccc(COc2ccccc2C[C@@H](Oc2ncnc3sc(-c4ccc(F)cc4)c(-c4ccc(OCCN5CC[N+](C)(Cc6ccc(O[C@@H]7O[C@H](C(=O)O)[C@@H](O)[C@H](O)[C@H]7O)c(NC(=O)CCNC(=O)[C@H](CCCCNC(=O)NCCOCCOCCOCCOCCOCCOCCOCCOCCOCCOCCOCCOCCOCCOCCOCCOCCOCCOCCOCCOCCOCCOCCOCCOCCC(=O)O)NC(=O)OCCN=[N+]=[N-])c6)CC5)c(Cl)c4C)c23)C(=O)O)n1]

A mixture of 1-(3-((S)-82-amino-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-iumtrifluoroacetate (45 mg, 0.016 mmol), 2-azidoethyl (4-nitrophenyl) carbonate (9.14 mg, 0.036 mmol) and DIPEA (0.011 mL, 0.065 mmol) in DMF (0.5 mL) was stirred at RT for 1 hr. The reaction mixture was concentrated, diluted with DMSO (2 ml), and purified by RP-ISCO. The product fractions were lyophilized to afford solid 1-(3-((S)-82-(((2-azidoethoxy)carbonyl)amino)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((S)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium (29 mg, 63.9% yield). HRMS: M⁺=2656.1799, RT=2.27 min (5 min acidic method).

### Synthesis of 4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-(((2S,3R,4S,5S,6S)-6-carboxy-3,4,5-trihydroxytetrahydro-2H-pyran-2-yl)oxy)-3-((S)-1-carboxy-82-(((2-(4-((5S,8S)-89-carboxy-8-((3-((5-((4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium-1-yl)methyl)-2-(((2S,3R,4S,5S,6S)-6-carboxy-3,4,5-trihydroxytetrahydro-2H-pyran-2-yl)oxy)phenyl)amino)-3-oxopropyl)carbamoyl)-5-(1-(2,5-dioxo-2,5-dihydro-1H-pyrrol-1-yl)-27-oxo-3,6,9,12,15,18,21,24-octaoxa-28-azadotriacontan-32-yl)-3,6,14-trioxo-2,18,21,24,27,30,33,36,39,42,45,48,51,54,57,60,63,66,69,72,75,78,81,84,87-pentacosaoxa-4,7,13,15-tetraazanonaoctacontyl)-1H-1,2,3-triazol-1-yl)ethoxy)carbonyl)amino)-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)benzyl)-1-methylpiperazin-1-ium trifluoroacetate P1-L27-P1

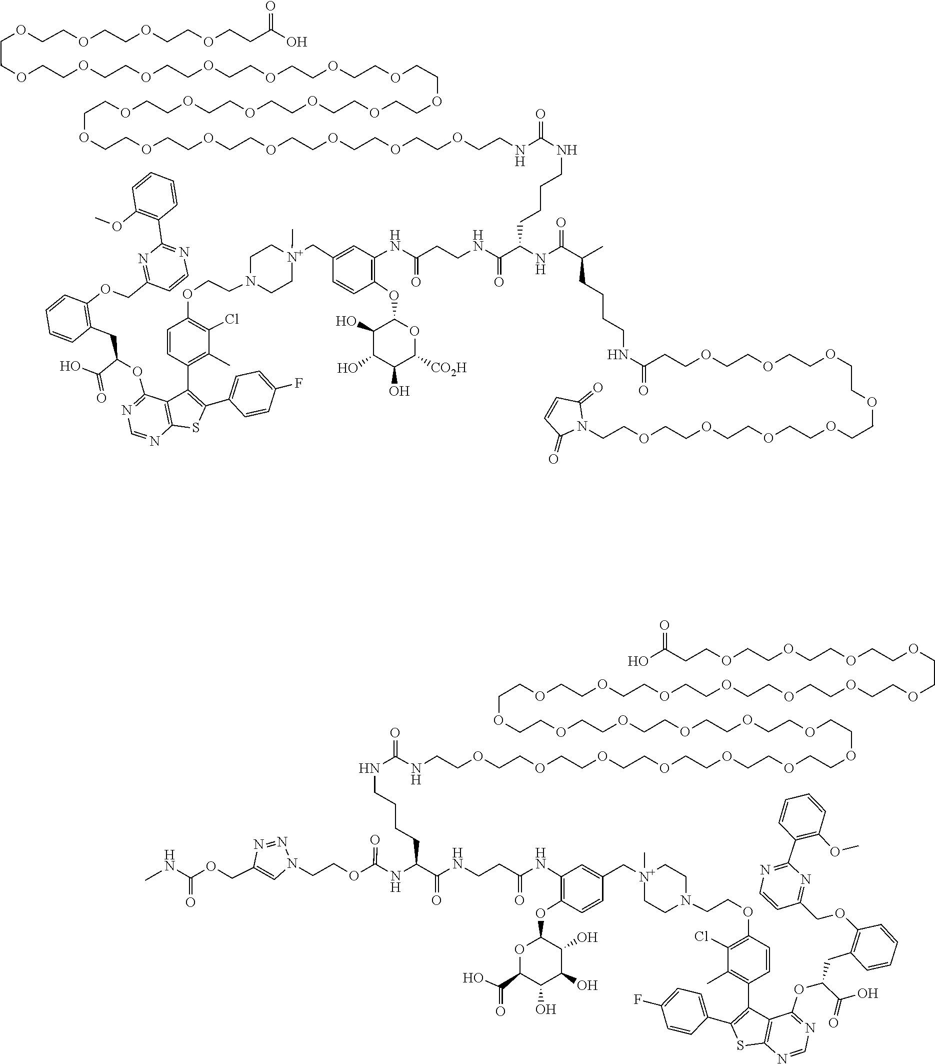

Following the GENERAL PROCEDURE #7 using 1-(3-((S)-82-(((2-azidoethoxy)carbonyl)amino)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (16.6 mg, 6.24 μmol), 1-(3-((S)-82-((S)-6-amino-2-(((prop-2-yn-1-yloxy)carbonyl)amino)hexanamido)-1-carboxy-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)-4-(((2S,3R,4S,5S,6S)-6-carboxy-3,4,5-trihydroxytetrahydro-2H-pyran-2-yl)oxy)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (17.1 mg, 5.96 μmol), sodium ascorbate 16 mg/mL in H₂O solution (130 μL, 10.4 μmol) and CuSO₄ pentahydrate 4 mg/mL H₂O solution (130 μL, 2.1 μmol), click product (15 mg, 43% yield) was obtained. HRMS: (M⁺²- H⁺)⁺=5408.4102, RT=2.33 min (5 min acidic method). Following the second part of GENERAL PROCEDURE #5 using isolated click product (14 mg, 2.6 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (4 mg, 6.5 μmol) and DIEA (6.7 mh, 52 μmol), 4-(2-(4-(4-((S)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-(((2S,3R,4S,5S,6S)-6-carboxy-3,4,5-trihydroxytetrahydro-2H-pyran-2-yl)oxy)-3-((S)-1-carboxy-82-(((2-(4-((5S,8S)-89-carboxy-8-((3-((5-((4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium-1-yl)methyl)-2-(((2S,3R,4S,5S,6S)-6-carboxy-3,4,5-trihydroxytetrahydro-2H-pyran-2-yl)oxy)phenyl)amino)-3-oxopropyl)carbamoyl)-5-(1-(2,5-dioxo-2,5-dihydro-1H-pyrrol-1-yl)-27-oxo-3,6,9,12,15,18,21,24-octaoxa-28-azadotriacontan-32-yl)-3,6,14-trioxo-2,18,21,24,27,30,33,36,39,42,45,48,51,54,57,60,63,66,69,72,75,78,81,84,87-pentacosaoxa-4,7,13,15-tetraazanonaoctacontyl)-1H-1,2,3-triazol-1-yl)ethoxy)carbonyl)amino)-76,83-dioxo-3,6,9,12,15,18,21,24,27,30,33,36,39,42,45,48,51,54,57,60,63,66,69,72-tetracosaoxa-75,77,84-triazaheptaoctacontan-87-amido)benzyl)-1-methylpiperazin-1-ium trifluoroacetate (6 mg, 36% yield) was obtained. HRMS: (M⁺²- H⁺)+=5911.6099, Rt=2.71 min (5 min acidic method).

### Synthesis of 1-(4-((S)-2-((S)-2-(((2-(4-((5S,8S,11S)-16-amino-5-(4-((tert-butoxycarbonyl)amino)butyl)-11-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)carbamoyl)-8-isopropyl-3,6,9,16-tetraoxo-2-oxa-4,7,10,15-tetraazahexadecyl)-1H-1,2,3-triazol-1-yl)ethoxy)carbonyl)amino)-3-methylbutanamido)-5-ureidopentanamido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

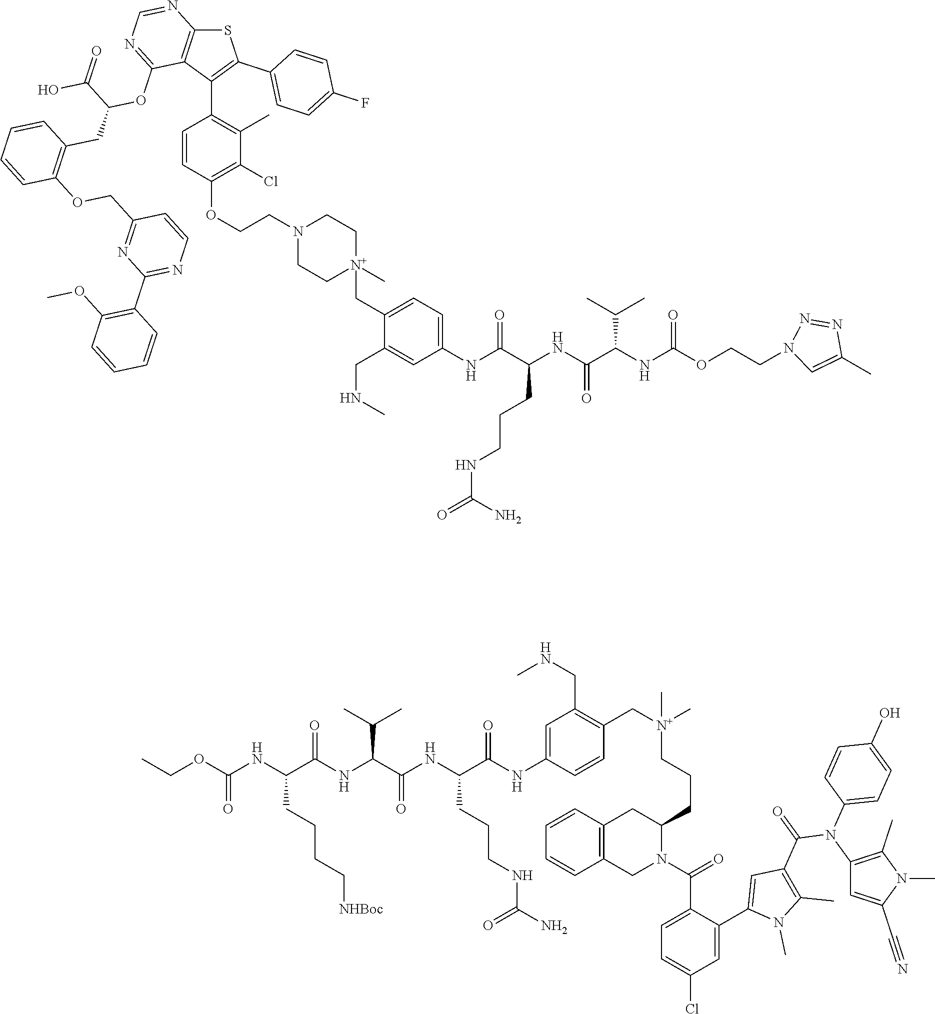

Following the GENERAL PROCEDURE #7 using 1-(4-((S)-2-((S)-2-(((2-azidoethoxy)carbonyl)amino)-3-methylbutanamido)-5-ureidopentanamido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (70 mg, 43 μmol), 3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N-(4-((1oS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)-2-((methylamino)methyl)benzyl)-N,N-dimethylpropan-1-aminium trifluoroacetate (78 mg, 47 μmol), sodium ascorbate 16 mg/mL in H₂O solution (801 μL, 65 μmol) and CuSO₄ pentahydrate 4 mg/mL H₂O solution (808 μL, 13 μmol), 1-(4-((S)-2-((S)-2-(((2-(4-((5S,8S,11S)-16-amino-5-(4-((tert-butoxycarbonyl)amino)butyl)-11-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)carbamoyl)-8-isopropyl-3,6,9,16-tetraoxo-2-oxa-4,7,10,15-tetraazahexadecyl)-1H-1,2,3-triazol-1-yl)ethoxy)carbonyl)amino)-3-methylbutanamido)-5-ureidopentanamido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (71 mg, 48% yield) was obtained. HRMS: (M⁺²- H⁺)+=2809.2500, Rt=2.24 min (5 min acidic method).

### Synthesis of 1-(4-((S)-2-((S)-2-(((2-(4-((5S,8S,11S)-16-amino-5-(4-((tert-butoxycarbonyl)amino)butyl)-11-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-8-isopropyl-3,6,9,16-tetraoxo-2-oxa-4,7,10,15-tetraazahexadecyl)-1H-1,2,3-triazol-1-yl)ethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

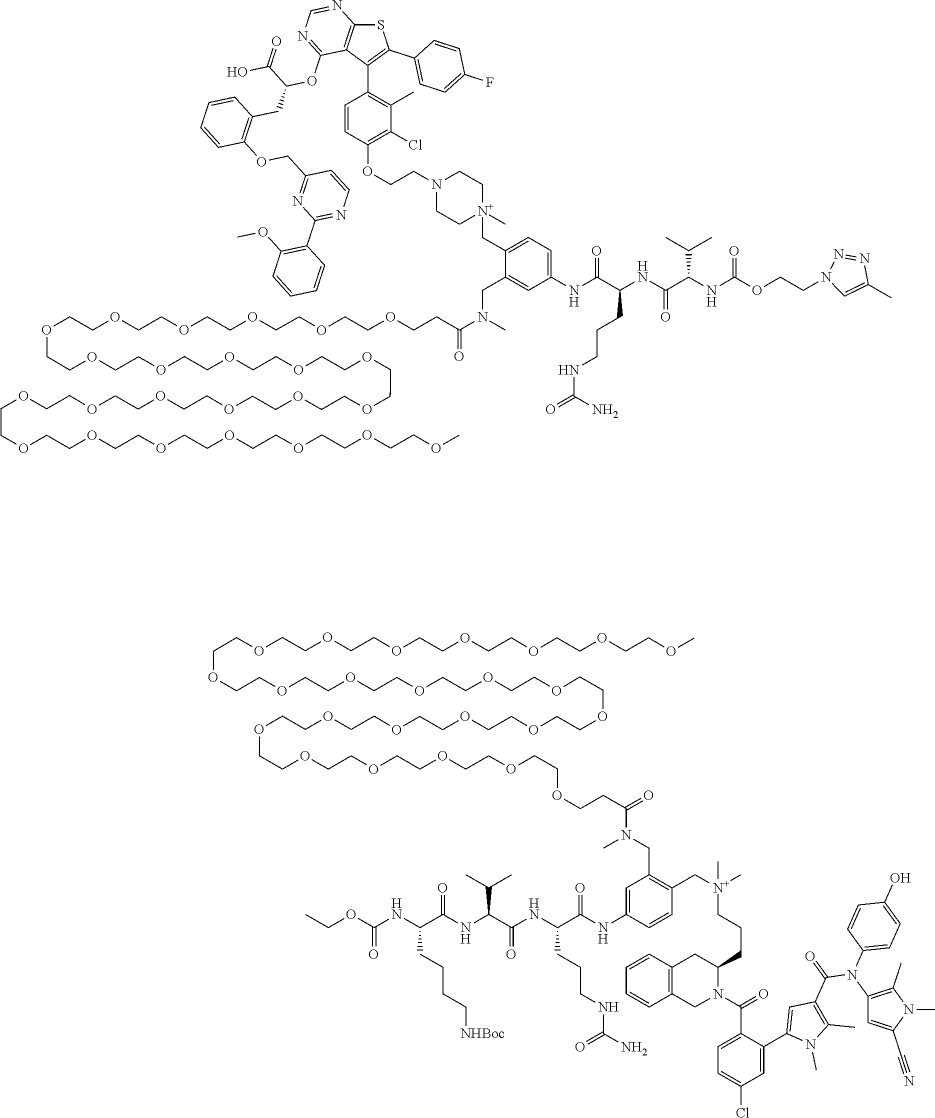

To a solution of 1-(4-((S)-2-((S)-2-(((2-(4-((5S,8S,11S)-16-amino-5-(4-((tert-butoxycarbonyl)amino)butyl)-11-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)carbamoyl)-8-isopropyl-3,6,9,16-tetraoxo-2-oxa-4,7,10,15-tetraazahexadecyl)-1H-1,2,3-triazol-1-yl)ethoxy)carbonyl)amino)-3-methylbutanamido)-5-ureidopentanamido)-2-((methylamino)methyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (71 mg, 22 μmol) and 2,5-dioxopyrrolidin-l-yl 2,5,8,11,14,17,20,23,26,29,32,35,38,41,44,47,50,53,56,59,62,65,68,71-tetracosaoxatetraheptacontan-74-oate (79 mg, 65 μmol) in DMF was added DIEA (30 μL, 172 μmol). After standing for 6 hours, the solution was diluted with DMSO and purified by RP-HPLC. After lyophilization, 1-(4-((S)-2-((S)-2-(((2-(4-((5S,8S,11S)-16-amino-5-(4-((tert-butoxycarbonyl)amino)butyl)-11-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-8-isopropyl-3,6,9,16-tetraoxo-2-oxa-4,7,10,15-tetraazahexadecyl)-1H-1,2,3-triazol-1-yl)ethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (89 mg, 76% yield) was obtained. HRMS: (M⁺²- H⁺)+=5006.5098, Rt=2.60 min (5 min acidic method).

### Synthesis of 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((S)-2-((S)-2-(((2-(4-((S)-5-(((S)-1-(((S)-1-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)carbamoyl)-37-(2,5-dioxo-2,5-dihydro-1H-pyrrol-1-yl)-3,11-dioxo-2,14,17,20,23,26,29,32,35-nonaoxa-4,10-diazaheptatriacontyl)-1H-1,2,3-triazol-1-yl)ethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (P1-L29-P2)

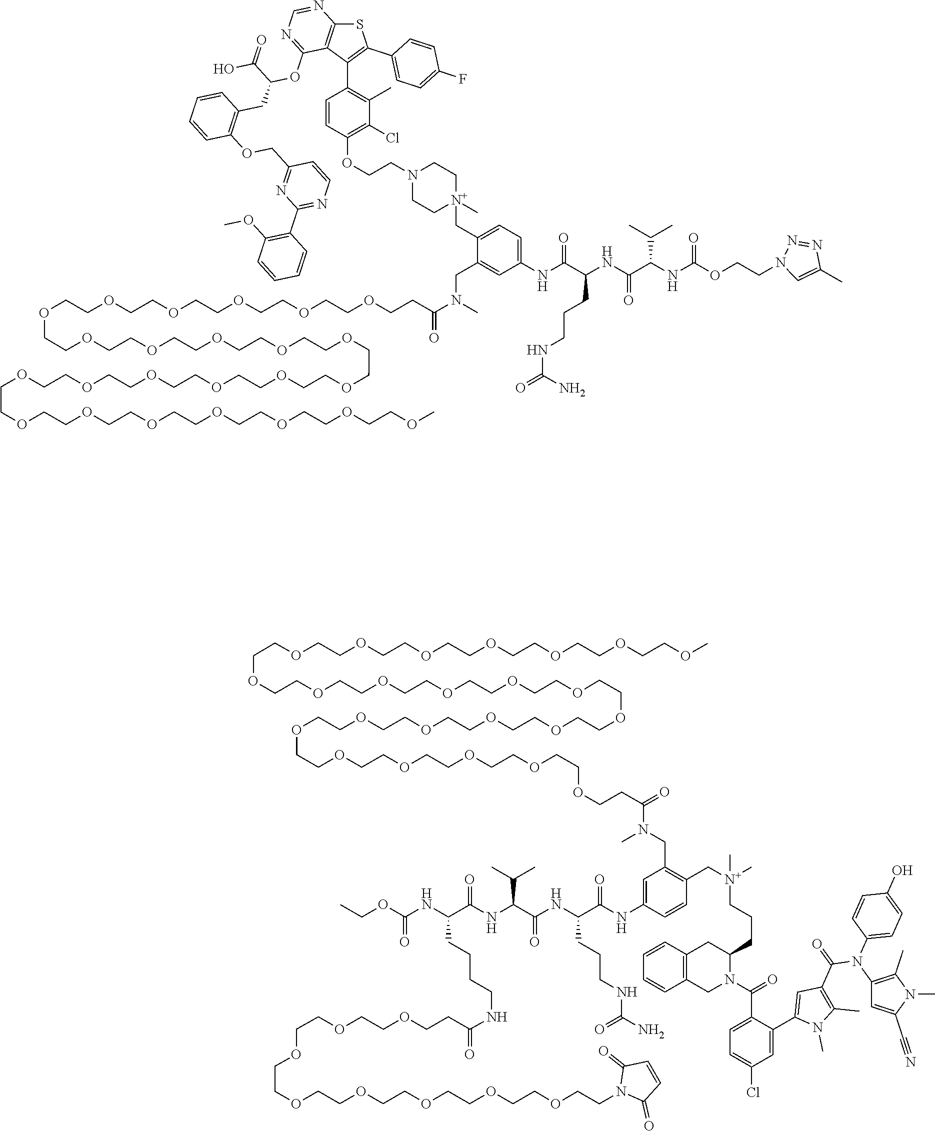

Following the GENERAL PROCEDURE #5 using 1-(4-((S)-2-((S)-2-(((2-(4-((5S,8S,11S)-16-amino-5-(4-((tert-butoxycarbonyl)amino)butyl)-11-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-8-isopropyl-3,6,9,16-tetraoxo-2-oxa-4,7,10,15-tetraazahexadecyl)-1H-1,2,3-triazol-1-yl)ethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (89 mg, 17 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (15 mg, 24 μmol) and DIEA (20 μL, 115 μmol), 4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-(4-((S)-2-((S)-2-(((2-(4-((S)-5-(((S)-1-(((S)-1-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)carbamoyl)-37-(2,5-dioxo-2,5-dihydro-1H-pyrrol-l-yl)-3,11-dioxo-2,14,17,20,23,26,29,32,35-nonaoxa-4,10-diazaheptatriacontyl)-1H-1,2,3-triazol-1-yl)ethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (32 mg, 33% yield) was obtained. HRMS: (M⁺²- H⁺)+=5409.6201, Rt=2.55 min (5 min acidic method).

### Synthesis of 1-(4-((5S,8S,11S)-1-(1-((6S,9S)-1-amino-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11-trioxo-12-oxa-2,7,10-triazatetradecan-14-yl)-1H-1,2,3-triazol-4-yl)-5-(4-aminobutyl)-8-isopropyl-3,6,9-trioxo-11-(3-ureidopropyl)-2-oxa-4,7,10-triazadodecan-12-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

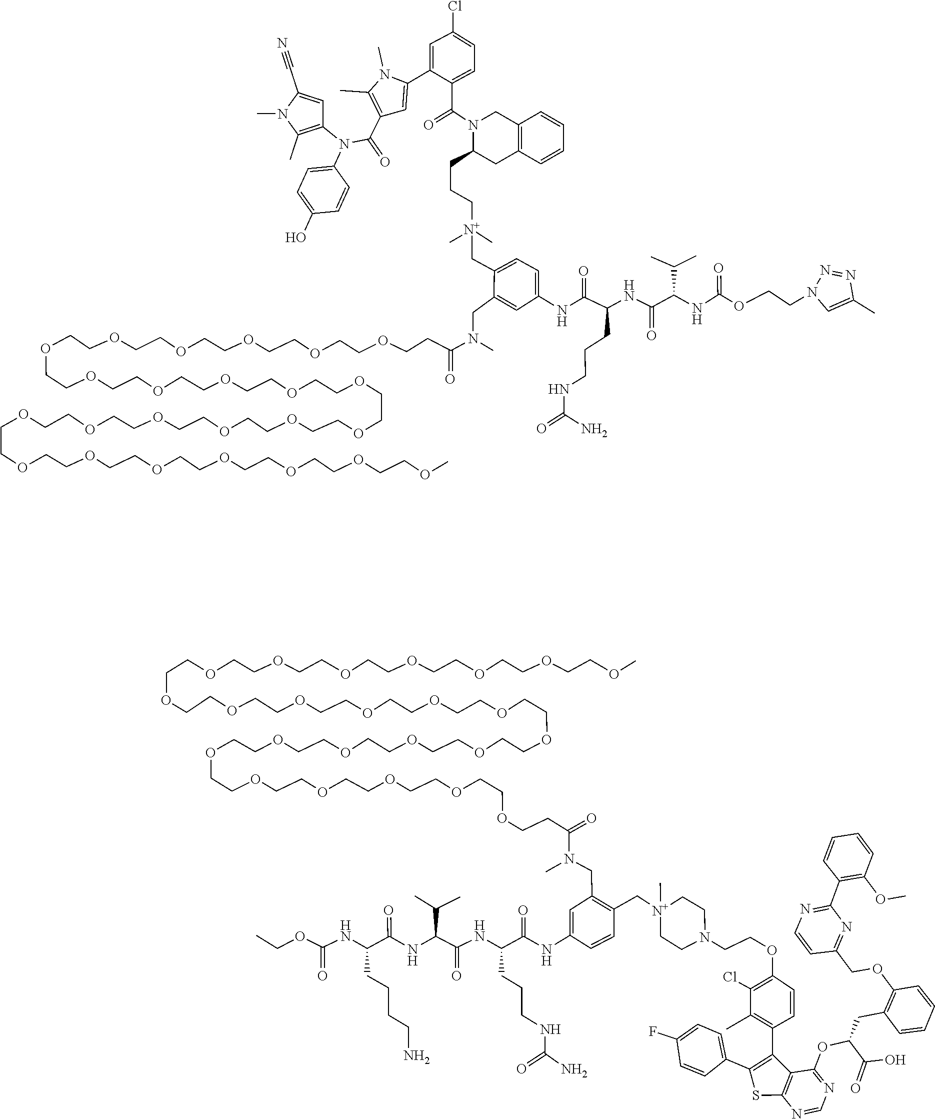

Following the GENERAL PROCEDURE #7 using 1-(4-((2S,5S,8S)-8-(4-aminobutyl)-5-isopropyl-4,7,10-trioxo-2-(3-ureidopropyl)-11-oxa-3,6,9-triazatetradec-13-ynamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (220 mg, 81 μmol), N-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (208 mg, 85 μmol), sodium ascorbate 16 mg/mL in H₂O solution (1511 μL, 122 μmol) and CuSO₄ pentahydrate 4 mg/mL H₂O solution (1523 μL, 24 μmol), 1-(4-((5S,8S,11S)-1-(1-((6S,9S)-1-amino-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11-trioxo-12-oxa-2,7,10-triazatetradecan-14-yl)-1H-1,2,3-triazol-4-yl)-5-(4-aminobutyl)-8-isopropyl-3,6,9-trioxo-11-(3-ureidopropyl)-2-oxa-4,7,10-triazadodecan-12-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (138 mg, 33% yield) was obtained. HRMS: (M⁺²- H⁺)+=4906.500, Rt=2.28 min (5 min acidic method).

### Synthesis of 1-(4-((33S,36S,39S)-33-((((1-((6S,9S)-1-amino-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11-trioxo-12-oxa-2,7,10-triazatetradecan-14-yl)-1H-1,2,3-triazol-4-yl)methoxy)carbonyl)amino)-1-(2,5-dioxo-2,5-dihydro-1H-pyrrol-1-yl)-36-isopropyl-27,34,37-trioxo-39-(3-ureidopropyl)-3,6,9,12,15,18,21,24-octaoxa-28,35,38-triazatetracontan-40-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P2-L29-P1)

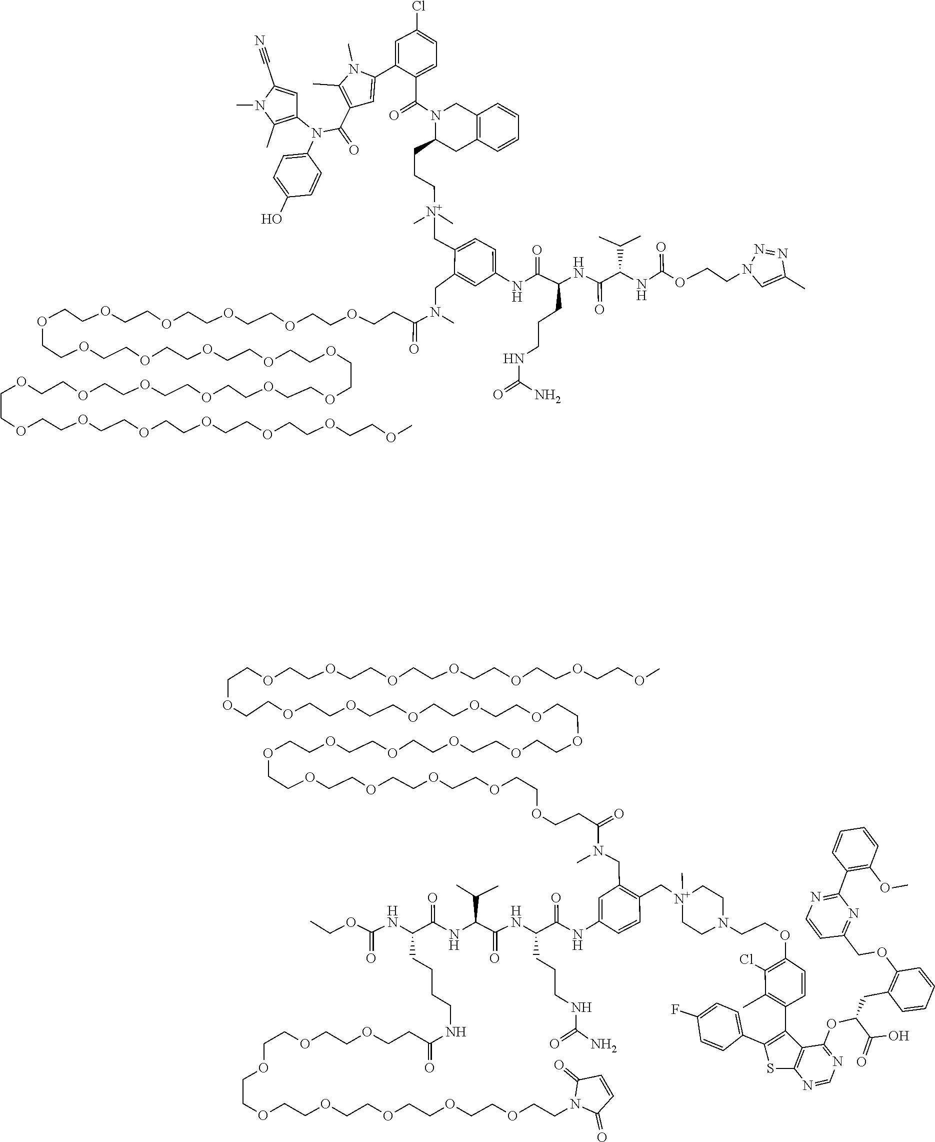

Following the second part of GENERAL PROCEDURE #5 and using 1-(4-((5S,8S,11S)-1-(1-((6S,9S)-1-amino-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11-trioxo-12-oxa-2,7,10-triazatetradecan-14-yl)-1H-1,2,3-triazol-4-yl)-5-(4-aminobutyl)-8-isopropyl-3,6,9-trioxo-11-(3-ureidopropyl)-2-oxa-4,7,10-triazadodecan-12-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (109 mg, 22 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (26.8 mg, 43 μmol) and DIEA (38 μL, 217 μmol), 1-(4-((33S,36S,39S)-33-((((1-((6S,9S)-1-amino-6-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11-trioxo-12-oxa-2,7,10-triazatetradecan-14-yl)-1H-1,2,3-triazol-4-yl)methoxy)carbonyl)amino)-1-(2,5-dioxo-2,5-dihydro-1H-pyrrol-yl)-36-isopropyl-27,34,37-trioxo-39-(3-ureidopropyl)-3,6,9,12,15,18,21,24-octaoxa-28,35,38-triazatetracontan-40-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (72 mg, 56% yield) was obtained. HRMS: (M⁺²- H⁺)+=5409.7002 Rt=2.54 min (5 min acidic method).

### Synthesis of 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((10S,13S,16S)-10-((((1-(2-(((S)-1-(((S)-1-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)amino)-1-oxo-5-ureidopentan-2-yl)amino)-3-methyl-1-oxobutan-2-yl)amino)-2-oxoethyl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-((methylamino)methyl)benzyl)-1-methylpiperazin-1-ium trifluoroacetate

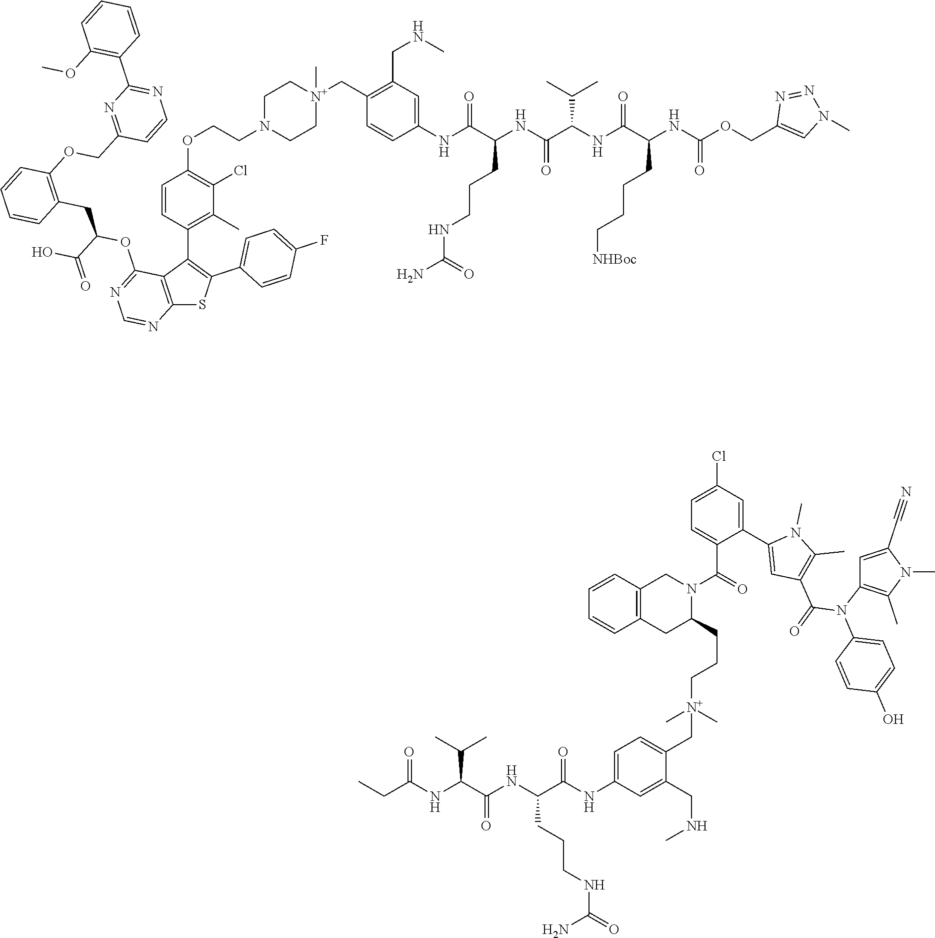

Following the GENERAL PROCEDURE #7 using 1-(2-(((((9H-fluoren-9-yl)methoxy)carbonyl)(methyl)amino)methyl)-4-((1oS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (116 mg, 60 μmol), N-(4-((S)-2-((S)-2-(2-azidoacetamido)-3-methylbutanamido)-5-ureidopentanamido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (87 mg, 61 μmol), sodium ascorbate 16 mg/mL in H₂O solution (1120 μL, 90 Pmol) and CuSO₄ pentahydrate 4 mg/mL H₂O solution (1130 μL, 81 μmol) and then adding 2M Me₂NH in MeOH (602 μL, 1204 μmol) and stirring for 1 hour before then purifying by RP-HPLC, 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((10S,13S,16S)-10-((((1-(2-(((S)-1-(((S)-1-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)amino)-1-oxo-5-ureidopentan-2-yl)amino)-3-methyl-1-oxobutan-2-yl)amino)-2-oxoethyl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-((methylamino)methyl)benzyl)-1-methylpiperazin-1-ium trifluoroacetate (76 mg, 39% yield) was obtained. HRMS: (M⁺²- H⁺)+=2779.2400, Rt=2.19 min (5 min acidic method).

### Synthesis of 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((1OS,13S,16S)-10-((((1-(2-(((S)-1-(((S)-1-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)amino)-2-oxoethyl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate

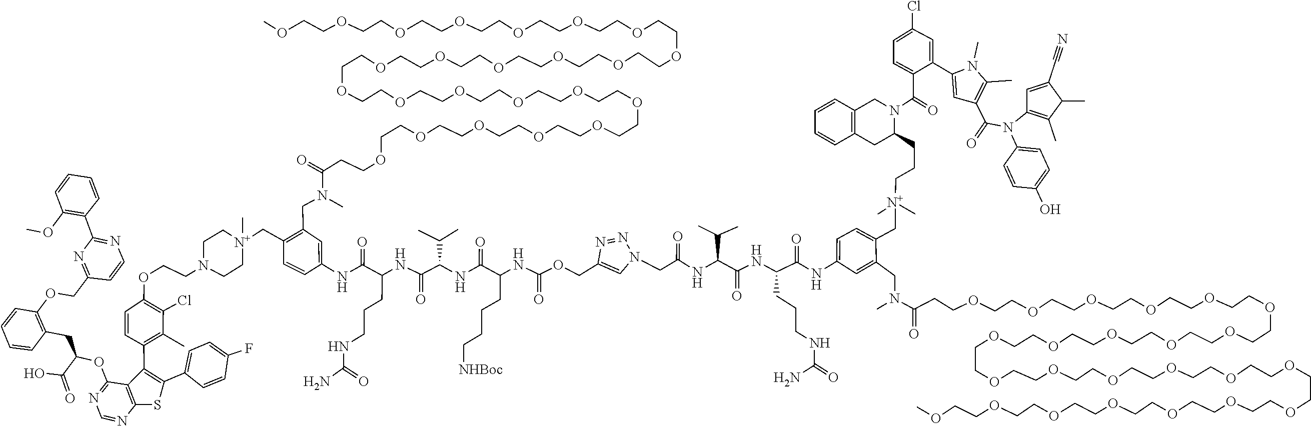

yl)methoxy)phenyl)ethoxy)-6-(4-fluorophenyl)thieno[2,3-d]pyrimidin-5-yl)-2-chloro-3-methylphenoxy)ethyl)-1-(4-((loS,13S,16S)-10-((((1-(2-(((S)-1-(((S)-1-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)amino)-1-oxo-5-ureidopentan-2-yl)amino)-3-methyl-1-oxobutan-2-yl)amino)-2-oxoethyl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-((methylamino)methyl)benzyl)-1-methylpiperazin-1-ium trifluoroacetate (76 mg, 23.4 μmol) and 2,5-dioxopyrrolidin-1-yl 2,5,8,11,14,17,20,23,26,29,32,35,38,41,44,47,50,53,56,59,62,65,68,71-tetracosaoxatetraheptacontan-74-oate (46 mg, 54 μmol) in DMF was added DIEA (30 μL, 173 μmol). After stirring for 20 hours, the solution was diluted with DMSO and purified by RP-HPLC. After lyophilization, 4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-(4-((10S,13S,16S)-10-((((1-(2-(((S)-1-(((S)-1-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)amino)-2-oxoethyl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (91 mg, 75% yield) was obtained. HRMS: (M⁺²- H⁺)+=4976.5098, Rt=2.57 min (5 min acidic method).

### Synthesis of 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((33S,36S,39S)-33-((((1-(2-(((S)-1-(((S)-1-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)amino)-2-oxoethyl)-1H-1,2,3-triazol-4-yl)methoxy)carbonyl)amino)-1-(2,5-dioxo-2,5-dihydro-1H-pyrrol-1-yl)-36-isopropyl-27,34,37-trioxo-39-(3-ureidopropyl)-3,6,9,12,15,18,21,24-octaoxa-28,35,38-triazatetracontan-40-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (P1-L30-P2)

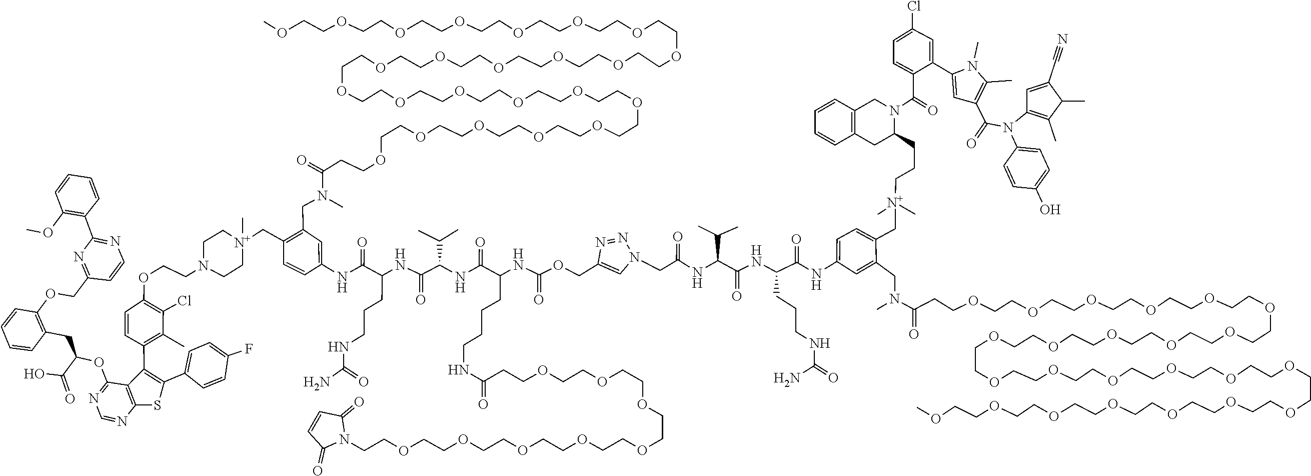

Following the GENERAL PROCEDURE #5 using 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((10S,13S,16S)-10-((((1-(2-(((S)-1-(((S)-1-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)amino)-2-oxoethyl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (91 mg, 18 μmol), 2,5-dioxopyrrolidin-l-yl 1-(2,5-dioxo-2,5-dihydro-1H-pyrrol-l-yl)-3,6,9,12,15,18,21,24-octaoxaheptacosan-27-oate (21.8 mg, 35 μmol) and DIEA (30 p L, 172 μmol), 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(4-((33S,36S,39S)-33-((((1-(2-(((S)-1-(((S)-1-((4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)amino)-2-oxoethyl)-1H-1,2,3-triazol-4-yl)methoxy)carbonyl)amino)-1-(2,5-dioxo-2,5-dihydro-1H-pyrrol-l-yl)-36-isopropyl-27,34,37-trioxo-39-(3-ureidopropyl)-3,6,9,12,15,18,21,24-octaoxa-28,35,38-triazatetracontan-40-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperazin-1-ium trifluoroacetate (52 mg, 48% yield) was obtained. HRMS: (M⁺²- H⁺)+=5379.7002, Rt=2.54 min (5 min acidic method).

### Synthesis of 1-(4-((5S,8S,11S)-1-(1-((6R,9R)-1-amino-6-((3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9-isopropyl-1,8,11-trioxo-12-oxa-2,7,10-triazatetradecan-14-yl)-1H-1,2,3-triazol-4-yl)-5-(4-aminobutyl)-8-isopropyl-3,6,9-trioxo-11-(3-ureidopropyl)-2-oxa-4,7,10-triazadodecan-12-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

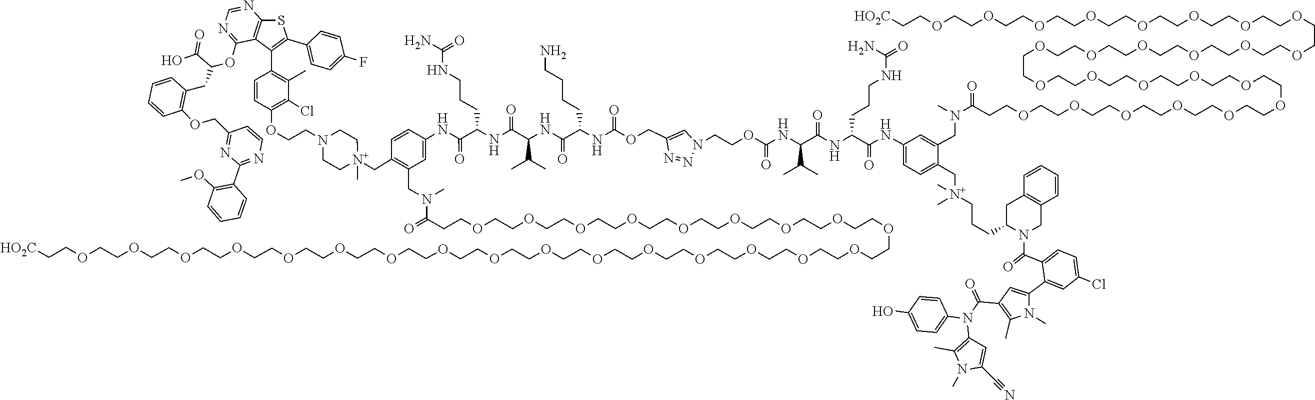

Following the GENERAL PROCEDURE #7 using 1-(4-((2S,5S,8S)-8-(4-aminobutyl)-5-isopropyl-4,7,10-trioxo-2-(3-ureidopropyl)-11-oxa-3,6,9-triazatetradec-13-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (64 mg, 23 μmol), N-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (60.8 mg, 25 μmol), sodium ascorbate 16 mg/mL in H₂O solution (424 μL, 34 μmol) and CuSO₄ pentahydrate 4 mg/mL H₂O solution (427 μL, 6.8 μmol), 1-(4-((5S,8S,11S)-1-(1-((6R,9R)-1-amino-6-((3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9-isopropyl-1,8,11-trioxo-12-oxa-2,7,10-triazatetradecan-l4-yl)-1H-1,2,3-triazol-4-yl)-5-(4-aminobutyl)-8-isopropyl-3,6,9-trioxo-11-(3-ureidopropyl)-2-oxa-4,7,10-triazadodecan-12-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (63 mg, 54% yield) was obtained. HRMS: (M⁺²- H⁺)⁺==5110.5098, Rt=2.32 min (5 min acidic method).

### Synthesis of 1-(4-((33S,36S,39S)-33-((((1-((6R,9R)-1-amino-6-((3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9-isopropyl-1,8,11-trioxo-12-oxa-2,7,10-triazatetradecan-14-yl)-1H-1,2,3-triazol-4-yl)methoxy)carbonyl)amino)-1-(2,5-dioxo-2,5-dihydro-1H-pyrrol-1-yl)-36-isopropyl-27,34,37-trioxo-39-(3-ureidopropyl)-3,6,9,12,15,18,21,24-octaoxa-28,35,38-triazatetracontan-40-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L31-P2)

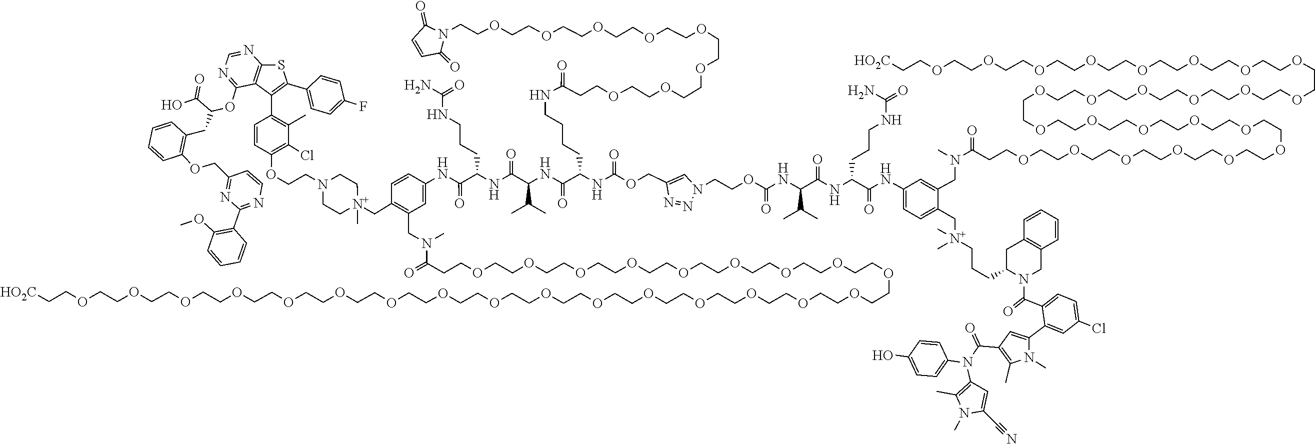

Following the second part of GENERAL PROCEDURE #5 using 1-(4-((S)-2-((S)-2-(((2-(4-((5S,8S,11S)-16-amino-5-(4-aminobutyl)-11-((3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-8-isopropyl-3,6,9,16-tetraoxo-2-oxa-4,7,10,15-tetraazahexadecyl)-1H-1,2,3-triazol-1-yl)ethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (67 mg, 13 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (15.9 mg, 26 μmol) and DIEA (18 μL, 103 μmol), 1-(4-((33S,36S,39S)-33-((((1-((6R,9R)-1-amino-6-((3-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-(((3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)phenyl)carbamoyl)-9-isopropyl-1,8,11-trioxo-12-oxa-2,7,10-triazatetradecan-l4-yl)-1H-1,2,3-triazol-4-yl)methoxy)carbonyl)amino)-1-(2,5-dioxo-2,5-dihydro-1H-pyrrol-l-yl)-36-isopropyl-27,34,37-trioxo-39-(3-ureidopropyl)-3,6,9,12,15,18,21,24-octaoxa-28,35,38-triazatetracontan-40-amido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (47 mg, 65% yield) was obtained. HRMS: (M⁺²- H⁺)+=5613.7598, Rt=2.46 min (5 min acidic method).

### Synthesis of 1-(4-((10S,13S,16S)-10-((((1-((6S,9S)-1-amino-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)carbamoyl)-9-isopropyl-1,8,11,14-tetraoxo-15-oxa-2,7,10,13-tetraazaheptadecan-l7-yl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

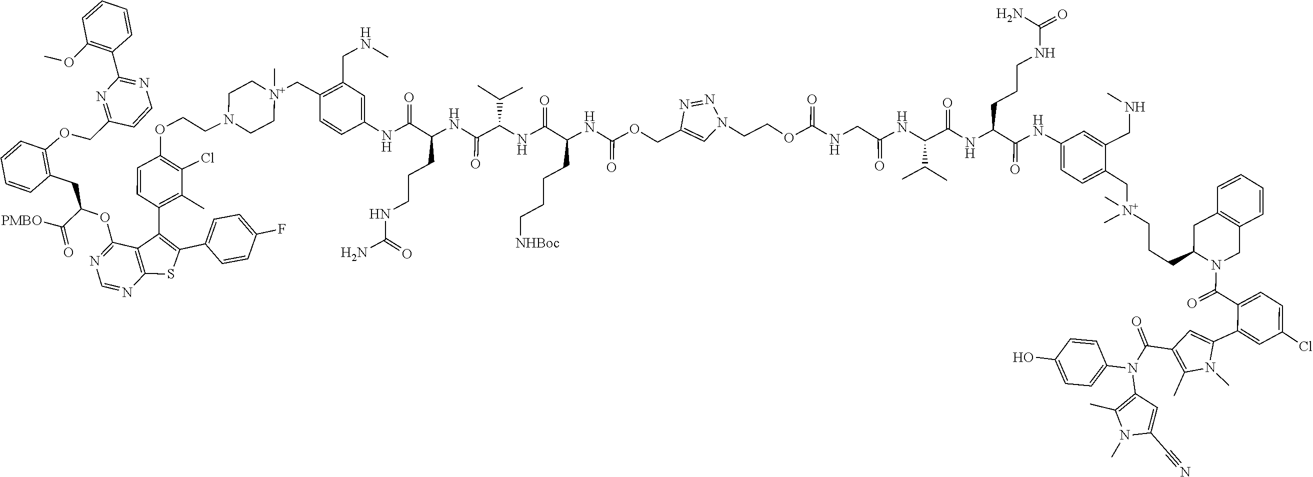

Following the GENERAL PROCEDURE #7 using 1-(2-(((((9H-fluoren-9-yl)methoxy)carbonyl)(methyl)amino)methyl)-4-((1OS,13S,16S)-13-isopropyl-2,2-dimethyl-4,11,14-trioxo-10-(((prop-2-yn-1-yloxy)carbonyl)amino)-16-(3-ureidopropyl)-3-oxa-5,12,15-triazaheptadecan-17-amido)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (64 mg, 33 μmol), N-(4-((9S,12S)-1-azido-9-isopropyl-4,7,10-trioxo-12-(3-ureidopropyl)-3-oxa-5,8,11-triazatridecan-13-amido)-2-((methylamino)methyl)benzyl)-3-((R)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)-N,N-dimethylpropan-1-aminium trifluoroacetate (42.3 mg, 33 μmol), sodium ascorbate 16 mg/mL in H₂O solution (615 μL, 50 μmol) and CuSO₄ pentahydrate 4 mg/mL H₂O solution (620 μL, 9.3 μmol) and then treating with 2M Me₂NH in MeOH (199 μL, 398 pM) for 2 hours before purifying by RP-HPLC, 1-(4-((10S,13S,16S)-10-((((1-((6S,9S)-1-amino-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)carbamoyl)-9-isopropyl-1,8,11,14-tetraoxo-15-oxa-2,7,10,13-tetraazaheptadecan-l7-yl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (34 mg, 34% yield) was obtained. HRMS: (M⁺²- H⁺)+=2986.3501, Rt=2.55 min (5 min acidic method).

### Synthesis of 1-(4-((10S,13S,16S)-10-((((1-((6S,9S)-1-amino-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11,14-tetraoxo-15-oxa-2,7,10,13-tetraazaheptadecan-l7-yl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate

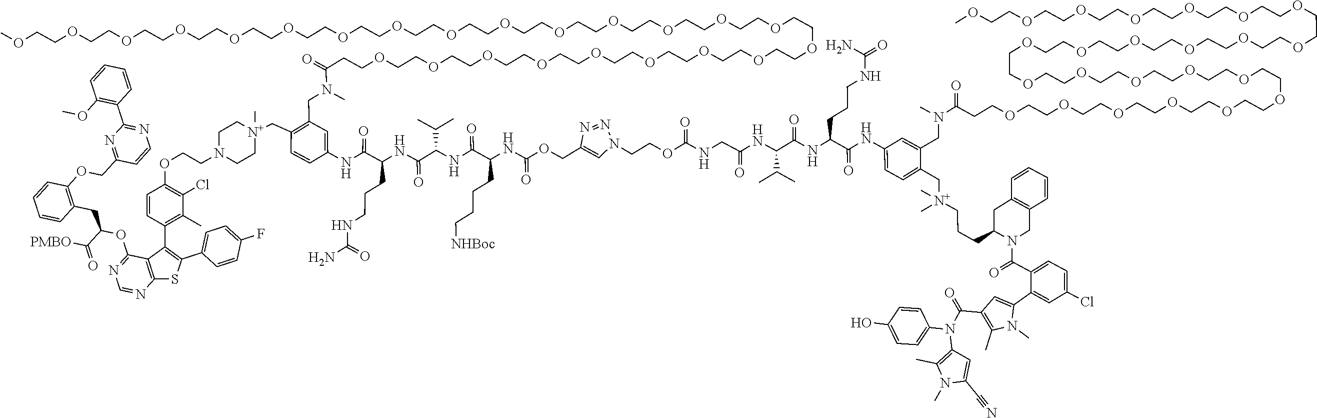

To a solution of 1-(4-((1OS,13S,16S)-10-((((1-((6S,9S)-1-amino-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-((methylamino)methyl)phenyl)carbamoyl)-9-isopropyl-1,8,11,14-tetraoxo-15-oxa-2,7,10,13-tetraazaheptadecan-l7-yl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-((methylamino)methyl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (56 mg, 19 μmol) and 2,5-dioxopyrrolidin-1-yl 2,5,8,11,14,17,20,23,26,29,32,35,38,41,44,47,50,53,56,59,62,65,68,71-tetracosaoxatetraheptacontan-74-oate (92 mg, 76 μmol) in DMF (0.75 mL) was added DIEA (26.2 μL, 150 μM). After stirring for four hours, the solution was diluted with DMSO and purified by RP-HPLC. After lyophilization, 1-(4-((10S,13S,16S)-10-((((1-((6S,9S)-1-amino-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11,14-tetraoxo-15-oxa-2,7,10,13-tetraazaheptadecan-17-yl)-1H-i,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[l2,3-d]pyrimidin-5-yl)-3-methylphenoxy)ethyl)-1-methylpiperazin-1-ium trifluoroacetate (22.2 mg, 23% yield) was obtained. HRMS: (M⁺²- H⁺)+=5183.6201, Rt=2.93 min (5 min acidic method).

### Synthesis of 1-(4-((33S,36S,39S)-33-((((1-((6S,9S)-1-amino-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11,14-tetraoxo-15-oxa-2,7,10,13-tetraazaheptadecan-17-yl)-1H-1,2,3-triazol-4-yl)methoxy)carbonyl)amino)-1-(2,5-dioxo-2,5-dihydro-1H-pyrrol-1-yl)-36-isopropyl-27,34,37-trioxo-39-(3-ureidopropyl)-3,6,9,12,15,18,21,24-octaoxa-28,35,38-triazatetracontan-40-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (P1-L32-P2)

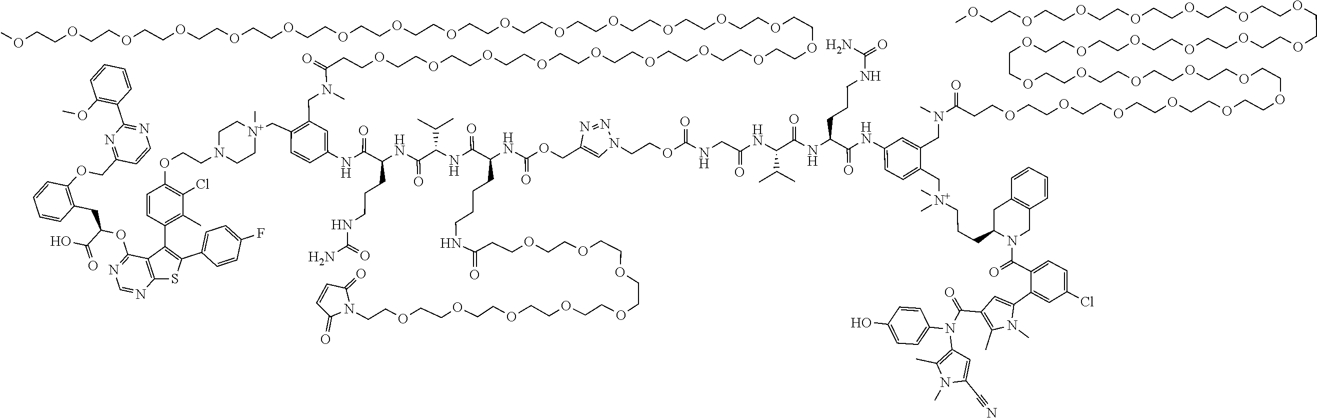

Following the GENERAL PROCEDURE #5 using 1-(4-((10S,13S,16S)-10-((((1-((6S,9S)-1-amino-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11,14-tetraoxo-15-oxa-2,7,10,13-tetraazaheptadecan-l7-yl)-1H-1,2,3-triazol-4-yl)methoxy)carbonyl)amino)-13-isopropyl-2,2-dimethyl-4,11,14-trioxo-16-(3-ureidopropyl)-3-oxa-5,12,15-triazaheptadecan-17-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(2-chloro-4-(6-(4-fluorophenyl)-4-(((R)-1-((4-methoxybenzyl)oxy)-3-(2-((2-(2-methoxyphenyl)pyrimidin-4-yl)methoxy)phenyl)-1-oxopropan-2-yl)oxy)thieno[2,3-d]pyrimidin-5-yl)-3-methylphenoxy)ethyl)-1-methylpiperazin-1-ium trifluoroacetate (22.2 mg, 4.2 μmol), 2,5-dioxopyrrolidin-1-yl 1-(2,5-dioxo-2,5-dihydro-1H-pyrrol-l-yl)-3,6,9,12,15,18,21,24-octaoxaheptacosan-27-oate (7.9 mg, 13 μmol) and DIEA (7.4 mg, 57 μmol), 1-(4-((33S,36S,39S)-33-((((1-((6S,9S)-1-amino-6-((4-(((3-((S)-2-(4-chloro-2-(4-((5-cyano-1,2-dimethyl-1H-pyrrol-3-yl)(4-hydroxyphenyl)carbamoyl)-1,5-dimethyl-1H-pyrrol-2-yl)benzoyl)-1,2,3,4-tetrahydroisoquinolin-3-yl)propyl)dimethylammonio)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-1,8,11,14-tetraoxo-15-oxa-2,7,10,13-tetraazaheptadecan-l7-yl)-1H-1,2,3-triazol-4-yl)methoxy)carbonyl)amino)-1-(2,5-dioxo-2,5-dihydro-1H-pyrrol-1-yl)-36-isopropyl-27,34,37-trioxo-39-(3-ureidopropyl)-3,6,9,12,15,18,21,24-octaoxa-28,35,38-triazatetracontan-40-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (16.2 mg, 63% yield) was obtained. HRMS: (M⁺²- H⁺)+=5466.6699, Rt=2.60 min (5 min acidic method).

### Synthesis of 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((26S,29S)-26-isopropyl-2,2-dimethyl-4,11,24,27-tetraoxo-12-(2-(2-(prop-2-yn-1-yloxy)ethoxy)ethyl)-29-(3-ureidopropyl)-3,8,15,18,21-pentaoxa-5,12,25,28-tetraazatriacontan-30-amido)benzyl)-1-methylpiperazin-1-ium trifluoroacetate

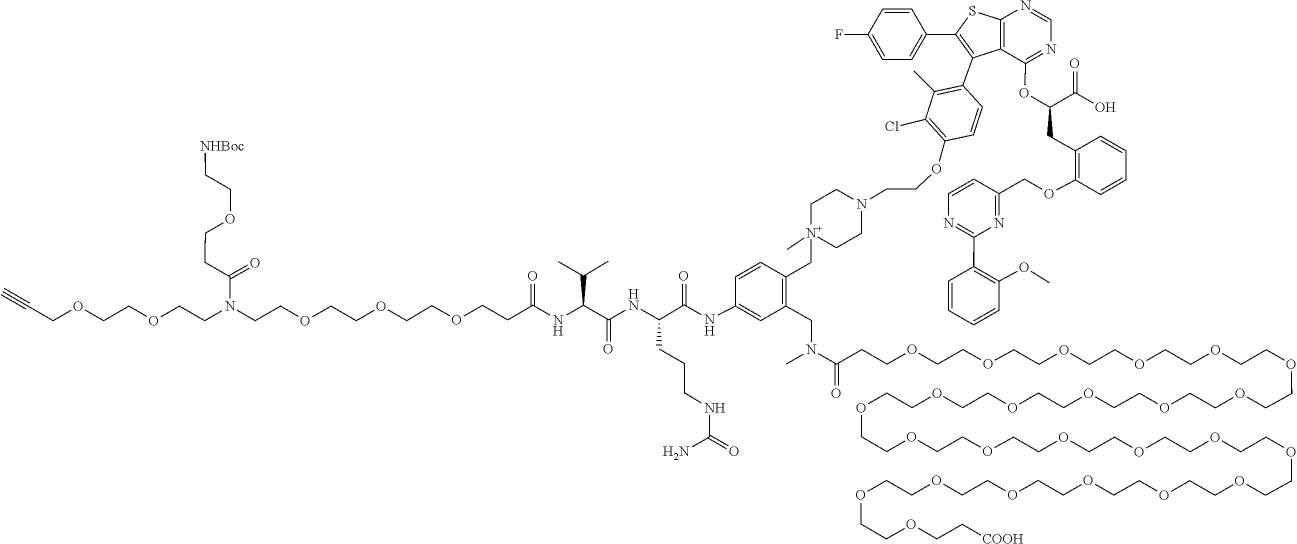 [SMILES: C#CCOCCOCCN(CCOCCOCCOCCC(=O)N[C@H](C(=O)N[C@@H](CCCNC(N)=O)C(=O)Nc1ccc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)c(CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)c1)C(C)C)C(=O)CCOCCNC(=O)OC(C)(C)C]

To a solution of 2,2-dimethyl-4,11-dioxo-12-(2-(2-(prop-2-yn-1-yloxy)ethoxy)ethyl)-3,8,15,18,21-pentaoxa-5,12-diazatetracosan-24-oic acid (44.8 mg, 80 μmol) and HATU (30.3 mg, 80 μmol) in DMF (0.8 mL) was added DIEA (84 μL, 478 μmol). After stirring for 30 minutes, 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (108 mg, 40 μmol) in DMF (3.5 mL) was added. After stirring for an additional 30 minutes, the solution was diluted with DMSO and purified by ISCO RP-HPLC. Upon lyophilization, 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((26S,29S)-26-isopropyl-2,2-dimethyl-4,11,24,27-tetraoxo-12-(2-(2-(prop-2-yn-1-yloxy)ethoxy)ethyl)-29-(3-ureidopropyl)-3,8,15,18,21-pentaoxa-5,12,25,28-tetraazatriacontan-30-amido)benzyl)-1-methylpiperazin-1-ium trifluoroacetate (92 mg, 73% yield) was obtained. HRMS: M⁺=3024.5200, Rt=2.62 min (5 min acidic method).

### Synthesis of 1-({4-[(2S)-2-{(2S)-2-[2-(4-{8-[(14S,17S)-22-Amino-17-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-12,15,22-trioxo-14-(propan-2-yl)-3,6,9-trioxa-13,16,21-triazadocosan-l-yl]-30-(2,5-dioxo-2,5-dihydro-1H-pyrrol-1-yl)-9,16-dioxo-2,5,12,19,22,25,28-heptaoxa-8,15-diazatriacontan-1-yl}-1H-1,2,3-triazol-1-yl)acetamido]-3-methylbutanamido}-5-carbamamidopentanamido]-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl}methyl)-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (P1-L33-P1)

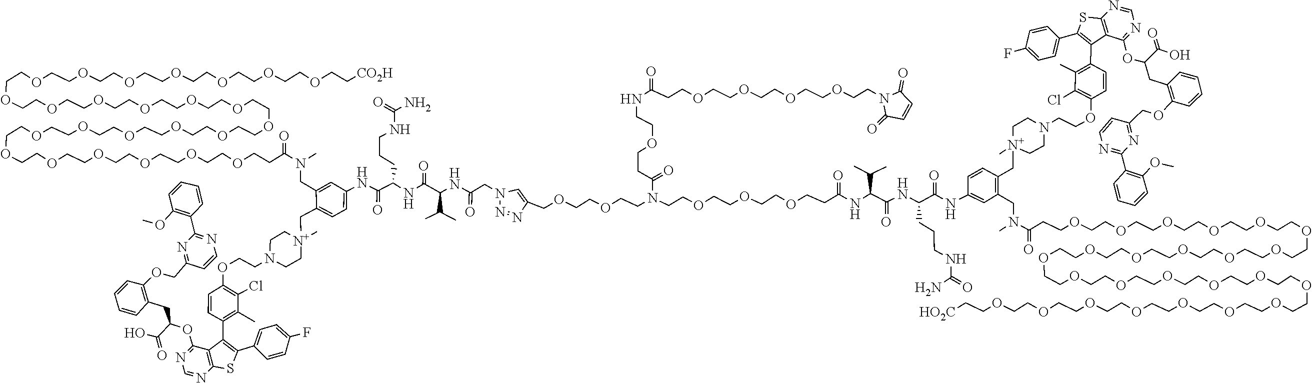

Following the GENERAL PROCEDURE #7 using 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((26S,29S)-26-isopropyl-2,2-dimethyl-4,11,24,27-tetraoxo-12-(2-(2-(prop-2-yn-1-yloxy)ethoxy)ethyl)-29-(3-ureidopropyl)-3,8,15,18,21-pentaoxa-5,12,25,28-tetraazatriacontan-30-amido)benzyl)-1-methylpiperazin-1-ium trifluoroacetate (55 mg, 18 μmol), 1-(4-((S)-2-((S)-2-(2-azidoacetamido)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium (54 mg, 21 μmol), sodium absorbate 32 mg/mL in H₂O solution (407 μL, 66 μmol) and CuSO₄ pentahydrate 16 mg/mL H₂O solution (342 μL, 22 μmol), click product (51.8 mg, 51% yield) was obtained. HRMS: (M⁺²- H⁺)⁺=5586.6602, Rt=2.67 min (5 min acidic method). Following the GENERAL PROCEDURE #5 using click product (51.8 mg, 8.9 μmol), 2,5-dioxopyrrolidin-l-yl 1-(2,5-dioxo-2,5-dihydro-1H-pyrrol-l-yl)-3,6,9,12-tetraoxapentadecan-15-oate (11.8 mg, 26.7 μmol) and DIEA (23.3 p L, 134 μmol), 1-({4-[(2S)-2-{(2S)-2-[2-(4-{8-[(14S,17S)-22-Amino-17-f{[4-({4-[2-(4-{4-[(1R)-1-carboxy-2-(2- {[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-l-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-12,15,22-trioxo-14-(propan-2-yl)-3,6,9-trioxa-13,16,21-triazadocosan-l-yl]-30-(2,5-dioxo-2,5-dihydro-1H-pyrrol-l-yl)-9,16-dioxo-2,5,12,19,22,25,28-heptaoxa-8,15-diazatriacontan-l-yl}-1H-1,2,3-triazol-1-yl)acetamido]-3-methylbutanamido}-5-carbamamidopentanamido]-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl}methyl)-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (25 mg, 46% yield) was obtained. HRMS: (M⁺²- H⁺)+=5813.7500, Rt=2.52 min (5 min acidic method).

### Synthesis of 1-{[4-{[(37S,40S)-23-{2-[2-({1-[(6S,9S)-14-Amino-9-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-4,7,14-trioxo-6-(propan-2-yl)-3-oxa-5,8,13-triazatetradecan-l-yl]-1H-1,2,3-triazol-4-yl}methoxy)ethoxy]ethyl}-40-(3-carbamamidopropyl)-1-(2,5-dioxo-2,5-dihydro-1H-pyrrol-1-yl)-15,22,35,38,41-pentaoxo-37-(propan-2-yl)-3,6,9,12,19,26,29,32-octaoxa-16,23,36,39-tetraazahentetracontan-41-yl]amino}-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]methyl}-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (P1-L34-P1)

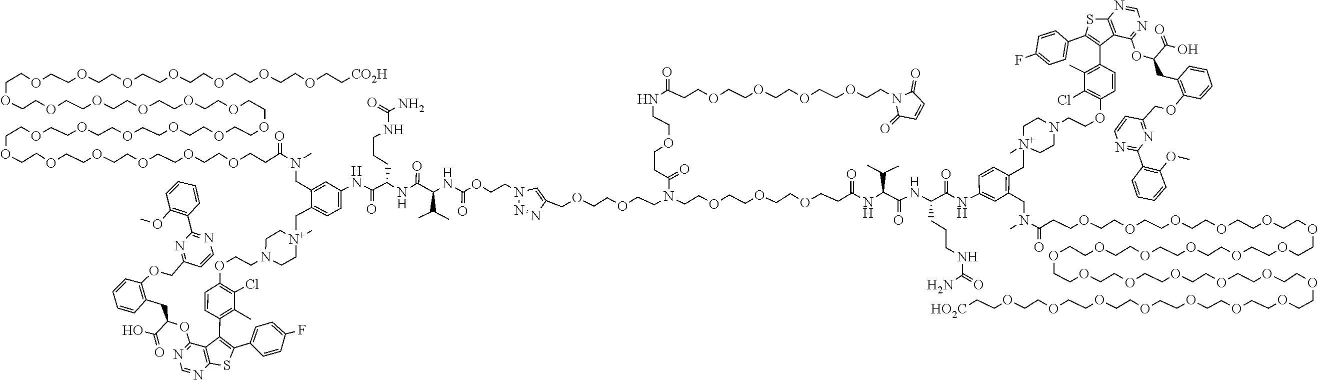

Following the GENERAL PROCEDURE #7 using 4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((26S,29S)-26-isopropyl-2,2-dimethyl-4,11,24,27-tetraoxo-12-(2-(2-(prop-2-yn-1-yloxy)ethoxy)ethyl)-29-(3-ureidopropyl)-3,8,15,18,21-pentaoxa-5,12,25,28-tetraazatriacontan-30-amido)benzyl)-1-methylpiperazin-1-ium trifluoroacetate (46 mg, 15 μmol), 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (47.6 mg, 18 μmol), sodium absorbate 32 mg/mL in H₂O solution (340 μL, 55 μmol) and CuSO₄ pentahydrate 16 mg/mL H₂O solution (286 μL, 18 μmol), click product (45 mg, 52% yield) was obtained. HRMS: (M⁺²- H⁺)+=5616.7300, Rt=2.70 min (5 min acidic method). Following the GENERAL PROCEDURE #5 using click product (32.3 mg, 5.5 μmol), 2,5-dioxopyrrolidin-1-yl 1-(2,5-dioxo-2,5-dihydro-1H-pyrrol-l-yl)-3,6,9,12-tetraoxapentadecan-15-oate (7.3 mg, 16.6 μmol) and DIEA (14.5 μL, 83 μmol), 1-{[4-{[(37S,40S)-23-{2-[2-({1-[(6S,9S)-14-Amino- 9- {[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-l-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-4,7,14-trioxo-6-(propan-2-yl)-3-oxa-5,8,13-triazatetradecan-l-yl]-1H-1,2,3-triazol-4-yl}methoxy)ethoxy]ethyl}-40-(3-carbamamidopropyl)-1-(2,5-dioxo-2,5-dihydro-1H-pyrrol-l-yl)-15,22,35,38,41-pentaoxo-37-(propan-2-yl)-3,6,9,12,19,26,29,32-octaoxa-16,23,36,39-tetraazahentetracontan-4l-yl]amino}-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]methyl}-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (14 mg, 41% yield) was obtained. HRMS: (M⁺²- H⁺)+=5843.7500, Rt=2.52 min (5 min acidic method).

### Synthesis of 1-(4-((2S,5S,18S)-18-(4-aminobutyl)-5-isopropyl-4,7,17,20-tetraoxo-2-(3-ureidopropyl)-10,13,23,26-tetraoxa-3,6,16,19-tetraazanonacos-28-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

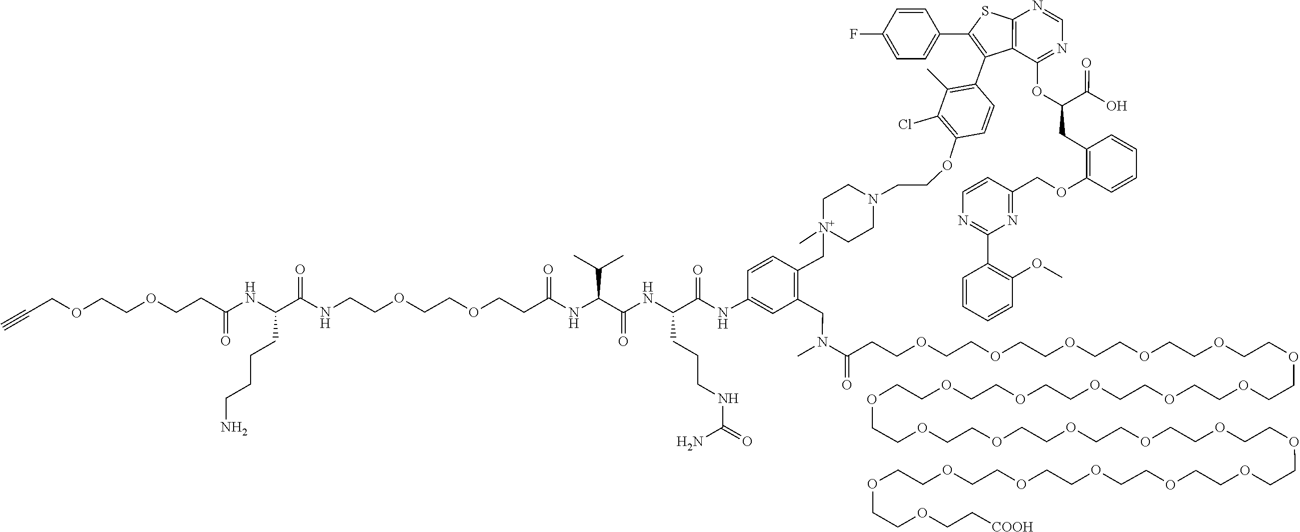 [SMILES: C#CCOCCOCCC(=O)N[C@@H](CCCCN)C(=O)NCCOCCOCCC(=O)N[C@H](C(=O)N[C@@H](CCCNC(N)=O)C(=O)Nc1ccc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)c(CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)c1)C(C)C]

Following the GENERAL PROCEDURE #6 using (S)-12-(4-((((9H-fluoren-9-yl)methoxy)carbonyl)amino)butyl)-11,14-dioxo-4,7,17,20-tetraoxa-10,13-diazatricos-22-ynoic acid (27.7 mg, 41 μmol), HATU (15.4 mg, 41 μmol), DIEA (49 μL, 284 μmol) and then 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (110 mg, 41 μmol); and then 2.0 M Dimethylamine in MeOH (406 μL, 812 μmol), 1-(4-((2S,5S,18S)-18-(4-aminobutyl)-5-isopropyl-4,7,17,20-tetraoxo-2-(3-ureidopropyl)-10,13,23,26-tetraoxa-3,6,16,19-tetraazanonacos-28-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate (101 mg, 79% yield) was obtained. HRMS: (M⁺+H⁺)+ 2/2=1462.2400, Rt=2.26 min (5 min acidic method).

### Synthesis of 1-{[4-{(2S)-2-[(2S)-2-{[(2-{4-[(10S,23S,26S)-31-Amino-26-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-10-[1-(2,5-dioxo-2,5-dihydro-1H-pyrrol-1-yl)-15-oxo-3,6,9,12-tetraoxa-16-azaicosan-20-yl]-8,11,21,24,31-pentaoxo-23-(propan-2-yl)-2,5,15,18-tetraoxa-9,12,22,25,30-pentaazahentriacontan-1-yl]-1H-1,2,3-triazol-1-yl}ethoxy)carbonyl]amino}-3-methylbutanamido]-5-carbamamidopentanamido}-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]methyl}-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (P1-L35-P1)

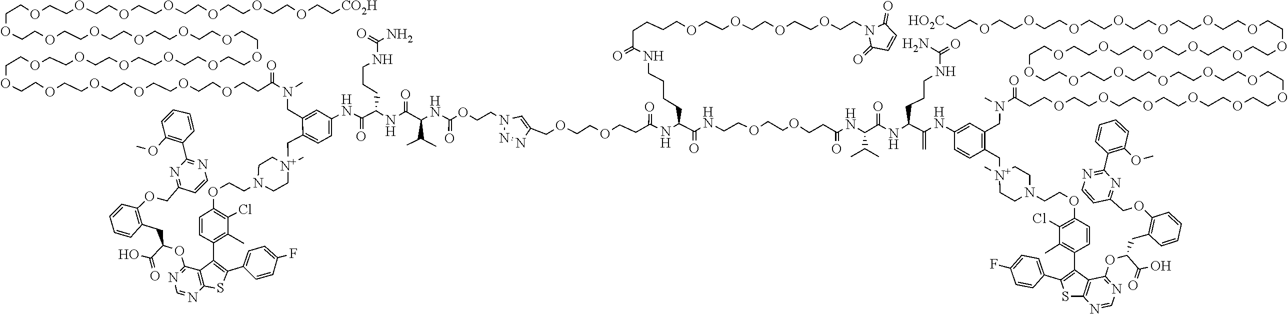

Following the GENERAL PROCEDURE #7 using 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (45.8 mg, 16 μmol), 1-(4-((2S,5S,18S)-18-(4-aminobutyl)-5-isopropyl-4,7,17,20-tetraoxo-2-(3-ureidopropyl)-10,13,23,26-tetraoxa-3,6,16,19-tetraazanonacos-28-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (44.5 mg, 14 μmol), sodium absorbate 16 mg/mL in H₂O solution (524 μL, 42 μmol) and CuSO₄ pentahydrate 4 mg/mL H₂O solution (881 μL, 14 μmol), click product (50 mg, 60% yield) was obtained. HRMS: (M⁺²- H⁺)+=5513.6602, Rt=2.53 min (5 min acidic method). Following the second part of GENERAL PROCEDURE #5 using click product (25.5 mg, 4.4 μmol), 2,5-dioxopyrrolidin-1-yl 1-(2,5-dioxo-2,5-dihydro-1H-pyrrol-l-yl)-3,6,9,12-tetraoxapentadecan-15-oate (2.9 mg, 6.5 μmol) and DIEA (7.6 p L, 44 μmol), 1-{[4-{(2S)-2-[(2S)-2-{[(2-{4-[(10S,23S,26S)-31-Amino-26-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-l-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-10-[1-(2,5-dioxo-2,5-dihydro-1H-pyrrol-l-yl)-15-oxo-3,6,9,12-tetraoxa-16-azaicosan-20-yl]-8,11,21,24,31-pentaoxo-23-(propan-2-yl)-2,5,15,18-tetraoxa-9,12,22,25,30-pentaazahentriacontan-l-yl]-1H-1,2,3-triazol-l-yl}ethoxy)carbonyl]amino)-3-methylbutanamido]-5-carbamamidopentanamido}-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]methyl}-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (19 mg, 72% yield) was obtained. HRMS: (M⁺²- H⁺)+=5840.8101, Rt=2.66 min (5 min acidic method).

### Synthesis of 1-(4-((2S,5S,21S)-21-(4-aminobutyl)-5-isopropyl-4,7,20,23-tetraoxo-2-(3-ureidopropyl)-10,13,16,26,29-pentaoxa-3,6,19,22-tetraazadotriacont-31-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno[2,3-d]pyrimidin-5-yl)-2-chloro-3-methylphenoxy)ethyl)-1-methylpiperazin-1-ium trifluoroacetate

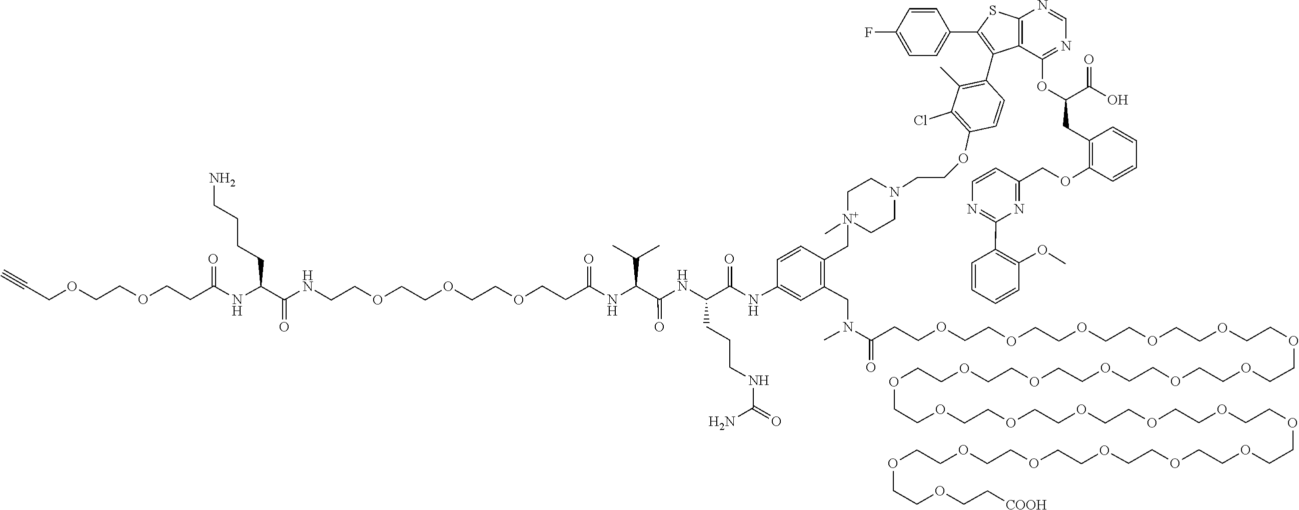 [SMILES: C#CCOCCOCCC(=O)N[C@@H](CCCCN)C(=O)NCCOCCOCCOCCC(=O)N[C@H](C(=O)N[C@@H](CCCNC(N)=O)C(=O)Nc1ccc(C[N+]2(C)CCN(CCOc3ccc(-c4c(-c5ccc(F)cc5)sc5ncnc(O[C@H](Cc6ccccc6OCc6ccnc(-c7ccccc7OC)n6)C(=O)O)c45)c(C)c3Cl)CC2)c(CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)c1)C(C)C]

Following the GENERAL PROCEDURE #6 using (S)-15-(4-((((9H-fluoren-9-yl)methoxy)carbonyl)amino)butyl)-14,17-dioxo-4,7,10,20,23-pentaoxa-13,16-diazahexacos-25-ynoic acid (37.1 mg, 51 μmol), HATU (19.4 mg, 51 μmol), DIEA (46.2 mg, 357 μmol) and 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (150 mg, 51 μmol); and then 2.0 M Dimethylamine in THF (510 μL, 1020 μmol), 1-(4-((2S,5S,21S)-21-(4-aminobutyl)-5-isopropyl-4,7,20,23-tetraoxo-2-(3-ureidopropyl)-10,13,16,26,29-pentaoxa-3,6,19,22-tetraazadotriacont-31-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (117 mg, 72% yield) was obtained. HRMS: (M⁺+H⁺)+ 2/2=1483.2400, Rt=2.15 min (5 min acidic method).

### Synthesis of 1-{[4-{(2S)-2-[(2S)-2-{[(2-{4-[(10S,26S,29S)-34-Amino-29-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-1-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-10-[1-(2,5-dioxo-2,5-dihydro-1H-pyrrol-1-yl)-15-oxo-3,6,9,12-tetraoxa-16-azaicosan-20-yl]-8,11,24,27,34-pentaoxo-26-(propan-2-yl)-2,5,15,18,21-pentaoxa-9,12,25,28,33-pentaazatetratriacontan-1-yl]-1H-1,2,3-triazol-1-yl}ethoxy)carbonyl]amino}-3-methylbutanamido]-5-carbamamidopentanamido}-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]methyl}-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (P1-L36-P1)

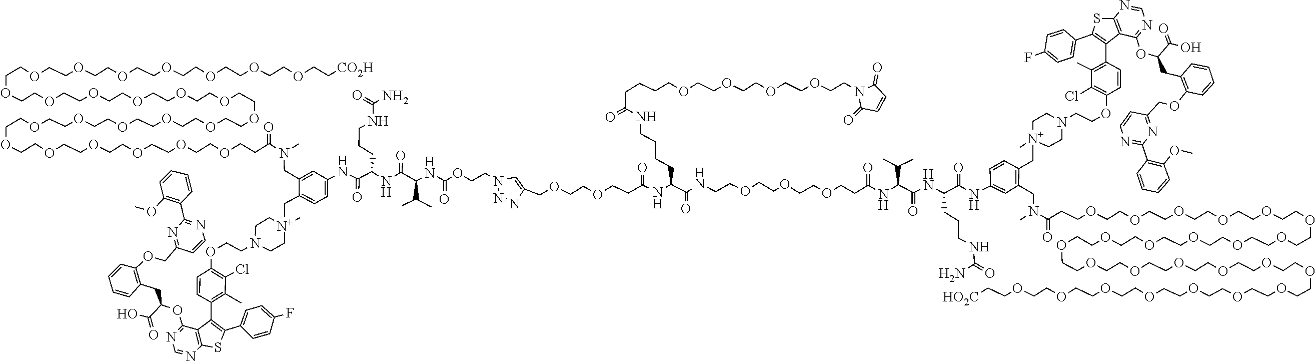

Following the GENERAL PROCEDURE #7 using 1-(4-((2S,5S,21S)-21-(4-aminobutyl)-5-isopropyl-4,7,20,23-tetraoxo-2-(3-ureidopropyl)-10,13,16,26,29-pentaoxa-3,6,19,22-tetraazadotriacont-31-ynamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (25 mg, 7.8 μmol), 1-(4-((S)-2-((S)-2-(((2-azidoethoxy)carbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-4-(2-(4-(4-((R)-1-carboxy-2-(2-((2-(2-methoxyphenyl)pyrimidin-4-yl)methoxy)phenyl)ethoxy)-6-(4-fluorophenyl)thieno [2,3-d]pyrimidin-5-yl)-2-chloro-3-methylphenoxy)ethyl)-1-methylpiperazin-1-ium trifluoroacetate (24.3 mg, 8.6 μmol), sodium absorbate 16 mg/mL in H₂O solution (291 μL, 23 μmol) and CuSO₄ pentahydrate 4 mg/mL H₂O solution (488 μL, 7.8 μmol), click product (20.6 mg, 45% yield) was obtained. HRMS: (M⁺²- H⁺)+=5557.6001, Rt=2.84 min (5 min acidic method). Following the second part of GENERAL PROCEDURE #5 using click product (20.6 mg, 3.5 μmol), 2,5-dioxopyrrolidin-1-yl 1-(2,5-dioxo-2,5-dihydro-1H-pyrrol-l-yl)-3,6,9,12-tetraoxapentadecan-15-oate (2.3 mg, 5.2 μmol) and DIEA (6 μL, 35 μmol), 1-{[4-{(2S)-2-[(2S)-2-{[(2-{4-[(10S,26S,29S)-34-Amino-29-{[4-({4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium-l-yl}methyl)-3-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]carbamoyl}-10-[1-(2,5-dioxo-2,5-dihydro-1H-pyrrol-l-yl)-15-oxo-3,6,9,12-tetraoxa-16-azaicosan-20-yl]-8,11,24,27,34-pentaoxo-26-(propan-2-yl)-2,5,15,18,21-pentaoxa-9,12,25,28,33-pentaazatetratriacontan-l-yl]-1H-1,2,3-triazol-l-yl}ethoxy)carbonyl]amino}-3-methylbutanamido]-5-carbamamidopentanamido}-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontan-1-yl)phenyl]methyl}-4-[2-(4-{4-[(1R)-1-carboxy-2-(2-{[2-(2-methoxyphenyl)pyrimidin-4-yl]methoxy}phenyl)ethoxy]-6-(4-fluorophenyl)thieno[2,3-d]pyrimidin-5-yl}-2-chloro-3-methylphenoxy)ethyl]-1-methylpiperazin-1-ium trifluoroacetate (18.6 mg, 89% yield) was obtained. HRMS: (M⁺²- H⁺)+=5884.8301, Rt=2.65 min (5 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate

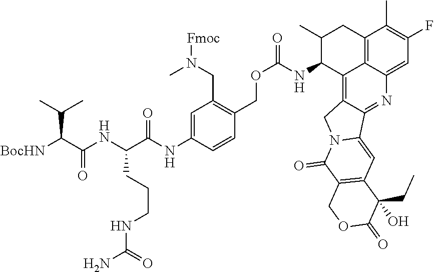

To a solution of (1S,9S)-1-amino-9-ethyl-5-fluoro-9-hydroxy-4-methyl-1,2,3,9,12,15-hexahydro-1OH,13H-benzo[de]pyrano[3′,4′:6,7]indolizino[1,2-b]quinoline-10,13-dione mesylate salt (1.5 g, 2,822 mmol) and (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (2.44 g, 2.68 mmol) in NMP (10 mL) was added DIEA (1133 μL, 7.07 mmol). After standing for 8 hours, the solution was diluted with DMSO and purified by RP-HPLC with 0.05% formic acid modifier. After lyophilization, (9H-fluoren-9-yl)methyl (5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (2.197 g, 64% yield) was obtained. HRMS: MH⁺=1206.5500, Rt=2.87 min (5 min acidic method).

### Synthesis of (9H-fluoren-9-yl)methyl (5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate

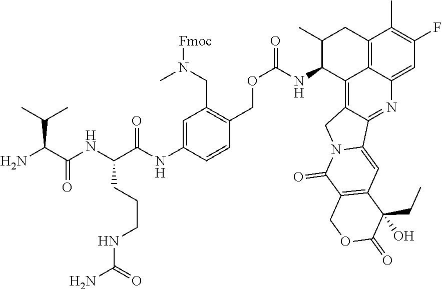

(9H-fluoren-9-yl)methyl (5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (2.197 g, 1.821 mmol) was treated with 20 mL of 25% TFA/CH₂Cl₂ with 1% Et₃SiH for one hour at which time the volatiles were removed in vacuo. The residue was triturated with Et₂O and pumped on to yield 2.07 grams (9H-fluoren-9-yl)methyl (5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate. The material was used directly as is. HRMS: MH⁺=1106.5000, Rt=2.09 min (5 min acidic method).

Synthesis of allyl (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oate

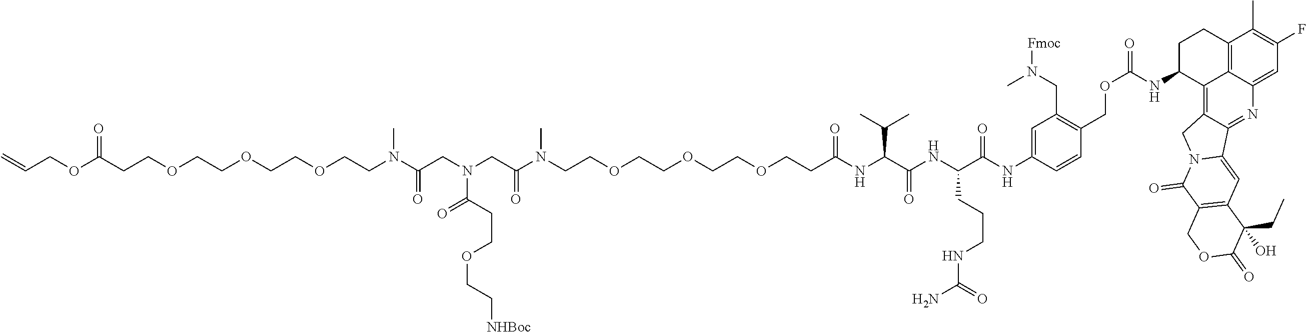

Following GENERAL PROCEDURE #3 using (9H-fluoren-9-yl)methyl (5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate TFA salt (2.07 g, 1696 μmol) and 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (1.565 g, 1902 μmol), allyl (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oate (1.80 g, 55% yield) was obtained. HRMS: MH⁺=1910.9200, Rt=1.85 min (5 min acidic method).

### Synthesis of (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oic acid

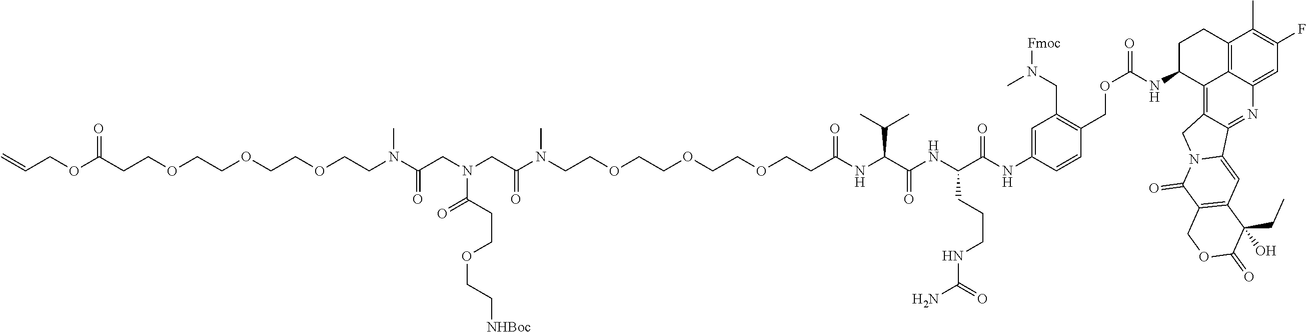

Following GENERAL PROCEDURE #2 using allyl (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oate (1.80 g, 942 μmol), phenyl silane (152.9 mg, 1413 μmol), tetrakis(triphenylphosphine)palladium (16.3 mg, 14.13 μmol) in 1:1 CH₂Cl₂/MeOH, (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oic acid (1.65 g, 93.6% yield) was obtained. HRMS: MH⁺=1870.8900, Rt=2.63 min (5 min acidic method).

### Synthesis of 3-(1-(((1r,3s,5R,7S)-3-(2-((((4-((6S,9S,43S,46S)-1-amino-26-(3-(2-((tert- butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-((methylamino)methyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid

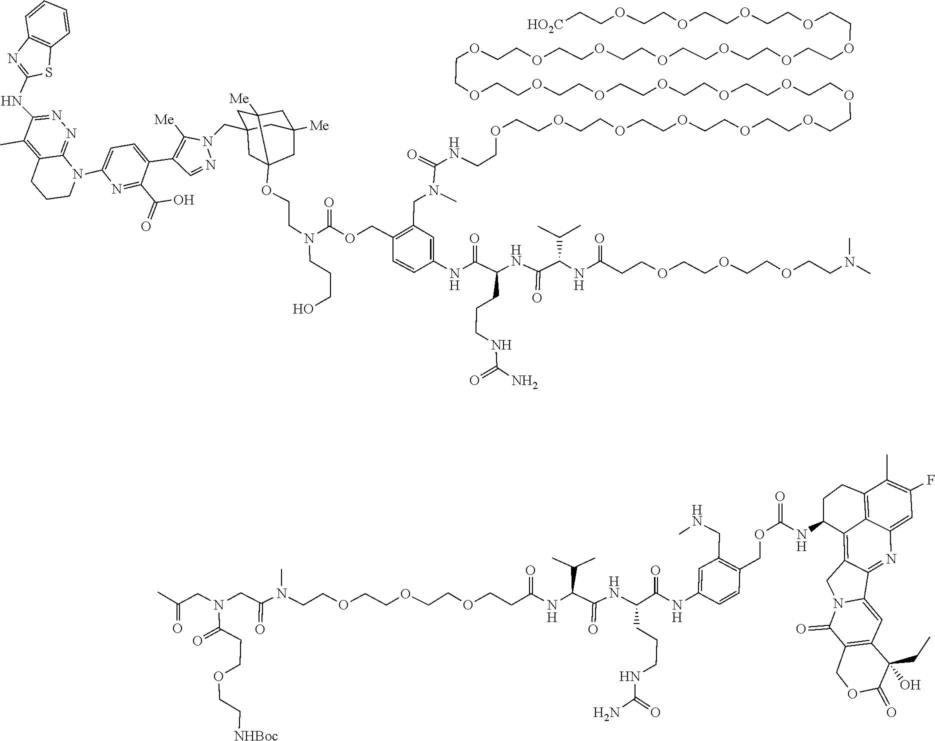

Following GENERAL PROCEDURE #4 using (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oic acid (1011 mg, 540.3 μmol), TSTU (162.7 mg, 540.3 μmol) and DIEA (235 μL, 1351 μmol), followed by 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid formic acid salt (1329 mg, 540.3 μmol) and DIEA (235 μL, 1351 μmol), followed then by in situ FMOC deprotection by addition of piperidine (534 μl, 5403 μmol), 3-(1-(((1r,3s,5R,7S)-3-(2-((((4-((6S,9S,43S,46S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-((methylamino)methyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (1310 mg, 55% yield) was obtained. HRMS: MH⁺=4042.1101, Rt=2.19 min (5 min acidic method).

### Synthesis of 3-(1-(((1r,3s,5R,7S)-3-(2-((((4-((6S,9S,43S,46S)-1-amino-26-(3-(2-((tert- butoxycarbonyl)amino)ethoxy)propanoyl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid

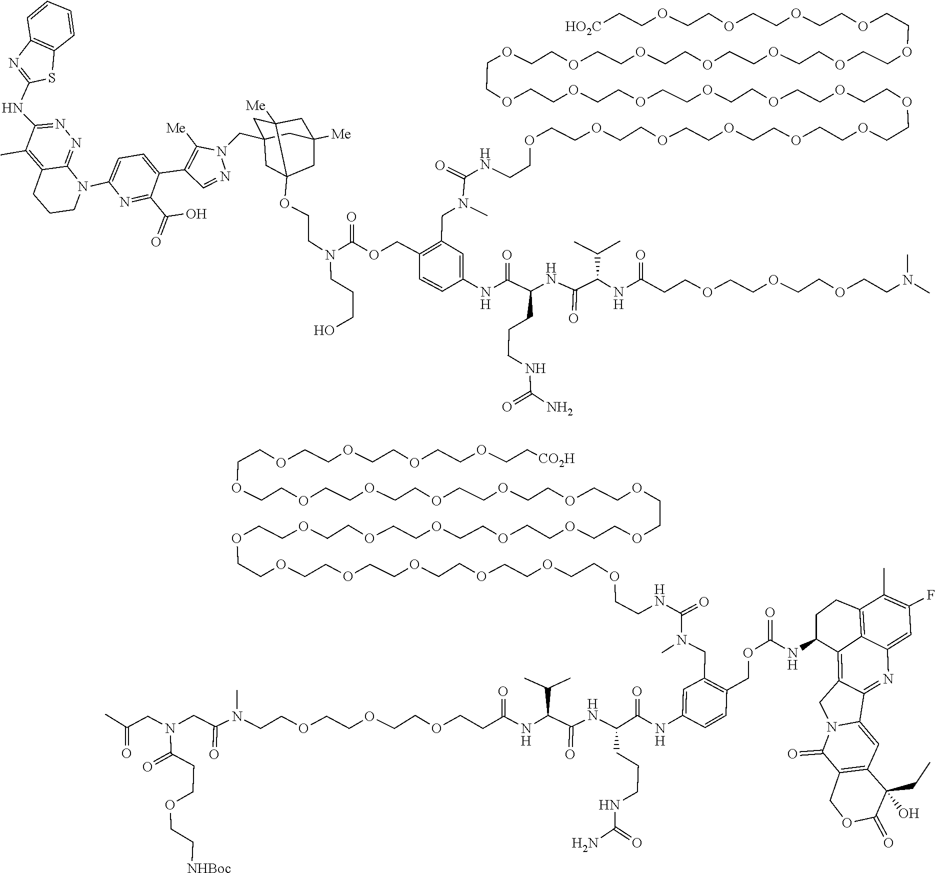

Following GENERAL PROCEDURE #1 using bis(4-nitrophenyl) carbonate (121.8 mg, 400.4 μmol), DIEA (251 μL, 1441 μmol), 1-amino-3,6,9,12,15,18,21,24,27,30,33,36,39,42,45,48,51,54,57,60,63,66,69,72-tetracosaoxapentaheptacontan-75-oic acid (186.3 mg, 251 μmol), and then 3-(1-(((1r,3s,5R,7S)-3-(2-((((4-((6S,9S,43S,46S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-((methylamino)methyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid TFA salt (1310 mg, 320.3 μmol) and DIEA (251 μL, 1441 μmol), 3-(1-(((1r,3s,5R,7S)-3-(2-((((4-((6S,9S,43S,46S)-1-amino-26-(3-(2-((tert-butoxycarbonyl)amino)ethoxy)propanoyl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (1438 mg, 85% yield) was obtained. HRMS: MH⁺=5213.87598, Rt=2.38 min (5 min acidic method).

### Synthesis of 3-(1-(((1r,3s,5R,7S)-3-(2-((((4-((6S,9S,43S,46S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid

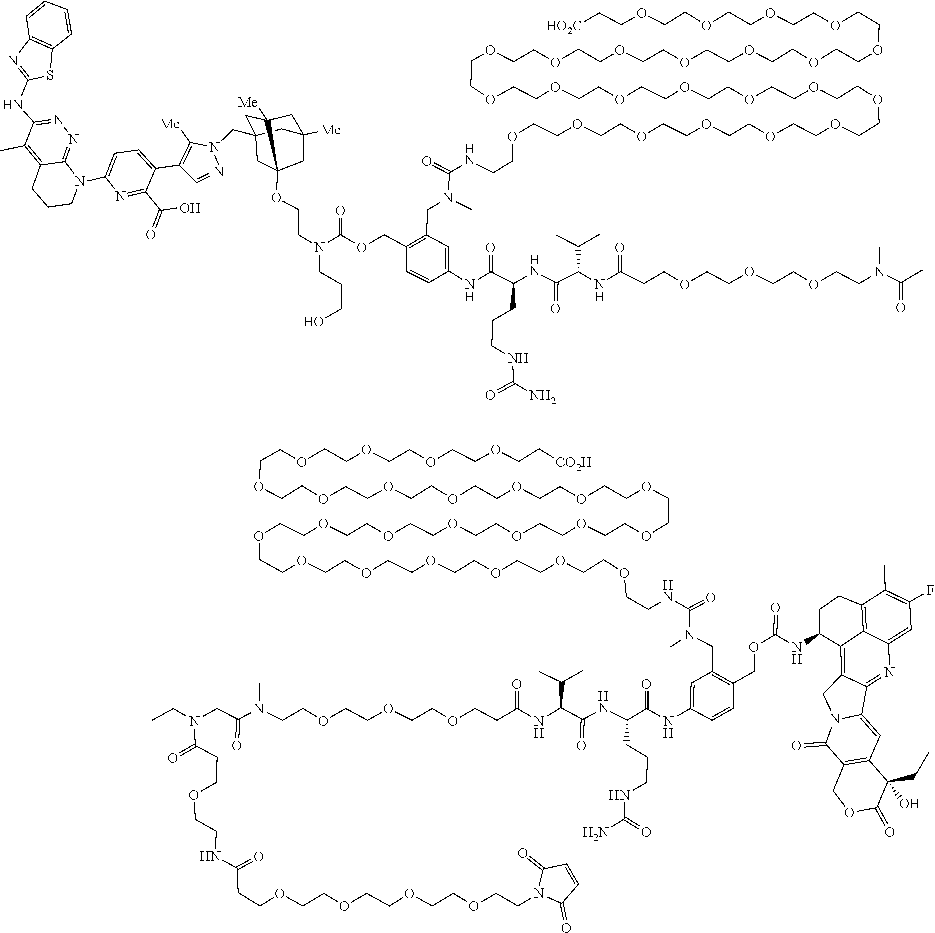

3-(1-(((1r,3s,5R,7S)-3-(2-((((4-((6S,9S,43S,46S)-1-amino-26-(3-(2-((tert-butoxycarbonyl)amino)ethoxy)propanoyl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (1.105 grams, 212 μmol) was treated with 18 ml of 25% TFA/CH2Cl2 with 1% Et₃SiH for one hour, and after removal of volatiles in vacuo the residue was dissolved in DMF (6 mL) and this solution was added to 2M Me₂NH in MeOH (2.65 mL, 5.3 mmol) to clip the trifluoroacetyl ester that had formed in addition to deBoc amine, hydroxyl alkyl product. After standing for 1 hour, the solution was diluted with DMSO and after purification by RP-HPLC with 0.05% formic acid modifier the deprotected amine hydroxy alkyl intermediate was isolated. Following the second part of GENERAL PROCEDURE #5 and using isolated amine formic acid salt (413.5 mg, 80.1 μmol), 2,5-dioxopyrrolidin-1-yl 1-(2,5-dioxo-2,5-dihydro-1H-pyrrol-l-yl)-3,6,9,12-tetraoxapentadecan-15-oate (49.6 mg, 112.1 μmol) and DIEA (70 μL, 401 μmol), 3-(1-(((1r,3s,5R,7S)-3-(2-((((4-((6S,9S,43S,46S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-26-(1-(2,5-dioxo-2,5-dihydro-1H-pyrrol-l-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (275 mg) was obtained after RP-HPLC with 0.05% formic acid modifier and lyophilization. HRMS: MH⁺=5440.8198, Rt=2.32 min (5 min acidic method).

### Synthesis of 4-(2-(((((9H-fluoren-9-yl)methoxy)carbonyl)(methyl)amino)methyl)-4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

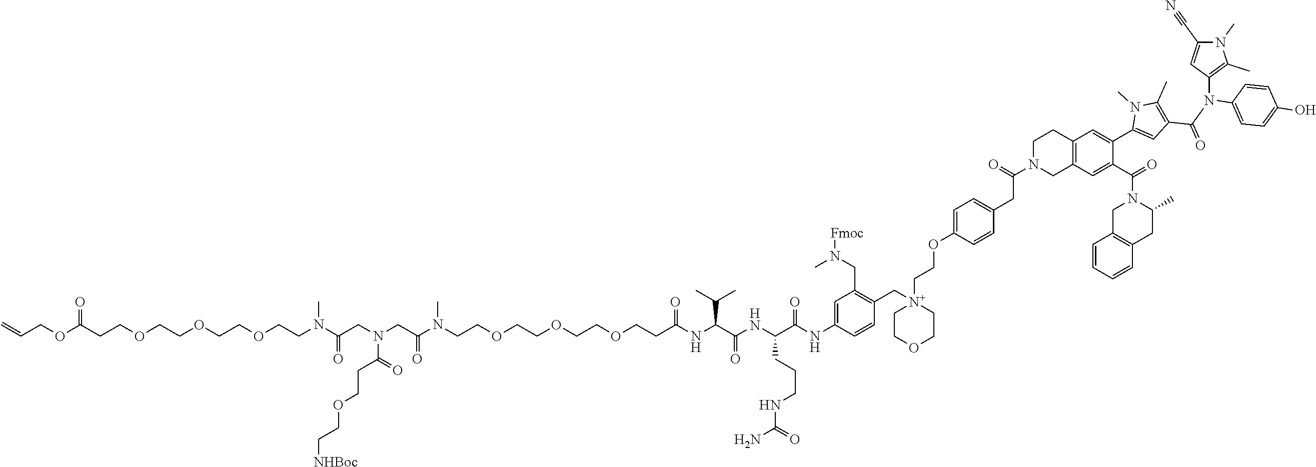

Following GENERAL PROCEDURE #3 using 4-(2-(((((9H-fluoren-9-yl)methoxy)carbonyl)(methyl)amino)methyl)-4-((S)-2-((S)-2-amino-3-methylbutanamido)-5-ureidopentanamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (277 mg, 168.7 μmol) and 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (145.8, 177.1 μmol), 4-(2-(((((9H-fluoren-9-yl)methoxy)carbonyl)(methyl)amino)methyl)-4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (284 mg, 68% yield) was obtained. HRMS: (M⁺Na⁺-H⁺)+=2353.2000 and (M-Boc)+=2231.1499, Rt=2.54 min (5 min acidic method).

### Synthesis of 4-(2-(((((9H-fluoren-9-yl)methoxy)carbonyl)(methyl)amino)methyl)-4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

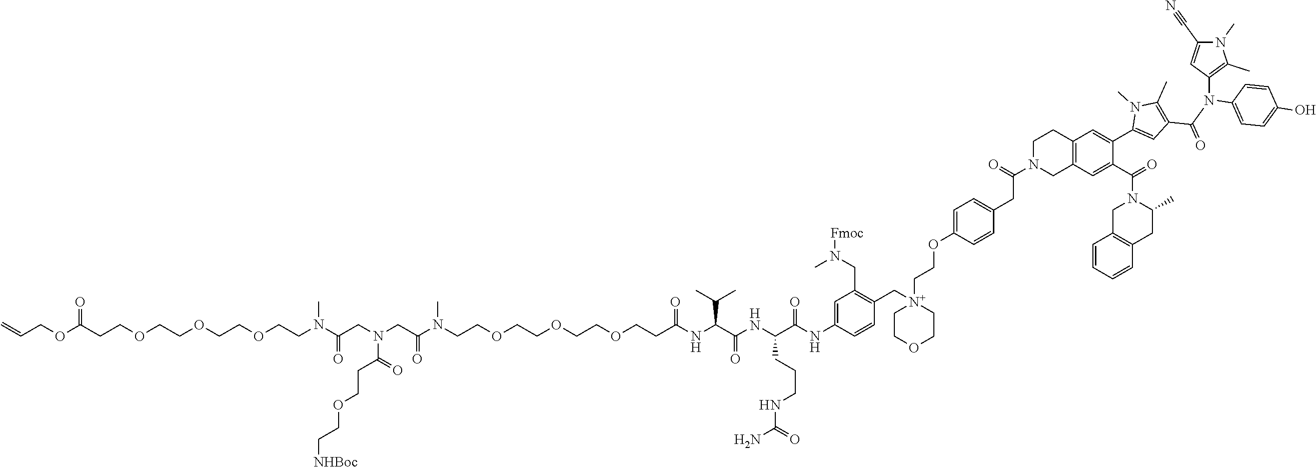

Following GENERAL PROCEDURE #2 using 4-(2-(((((9H-fluoren-9-yl)methoxy)carbonyl)(methyl)amino)methyl)-4-((2S,5S)-22-(3-(2-((tert-butoxycarbonyl)amino)ethoxy)propanoyl)-5-isopropyl-19,25-dimethyl-4,7,20,24,37-pentaoxo-2-(3-ureidopropyl)-10,13,16,28,31,34,38-heptaoxa-3,6,19,22,25-pentaazahentetracont-40-enamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (284, 121.7 μmol), phenyl silane (13.2 mg, 121.7 μmol), tetrakis(triphenylphosphine)palladium (8.4 mg, 7.4 μmol) in 1:1 CH₂Cl₂/MeOH, 4-(2-(((((9H-fluoren-9-yl)methoxy)carbonyl)(methyl)amino)methyl)-4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (181 mg, 64% yield) was obtained. HRMS: (M⁺Na⁺-H⁺)⁺=2313.1499 and (M-Boc)+=2191.1201, Rt=2.35 min (5 min acidic method).

### Synthesis of 4-(2-(((((9H-fluoren-9-yl)methoxy)carbonyl)(methyl)amino)methyl)-4-((6S,9S,43S,46S)-1-amino-6-((4-((((2-(((ls,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate

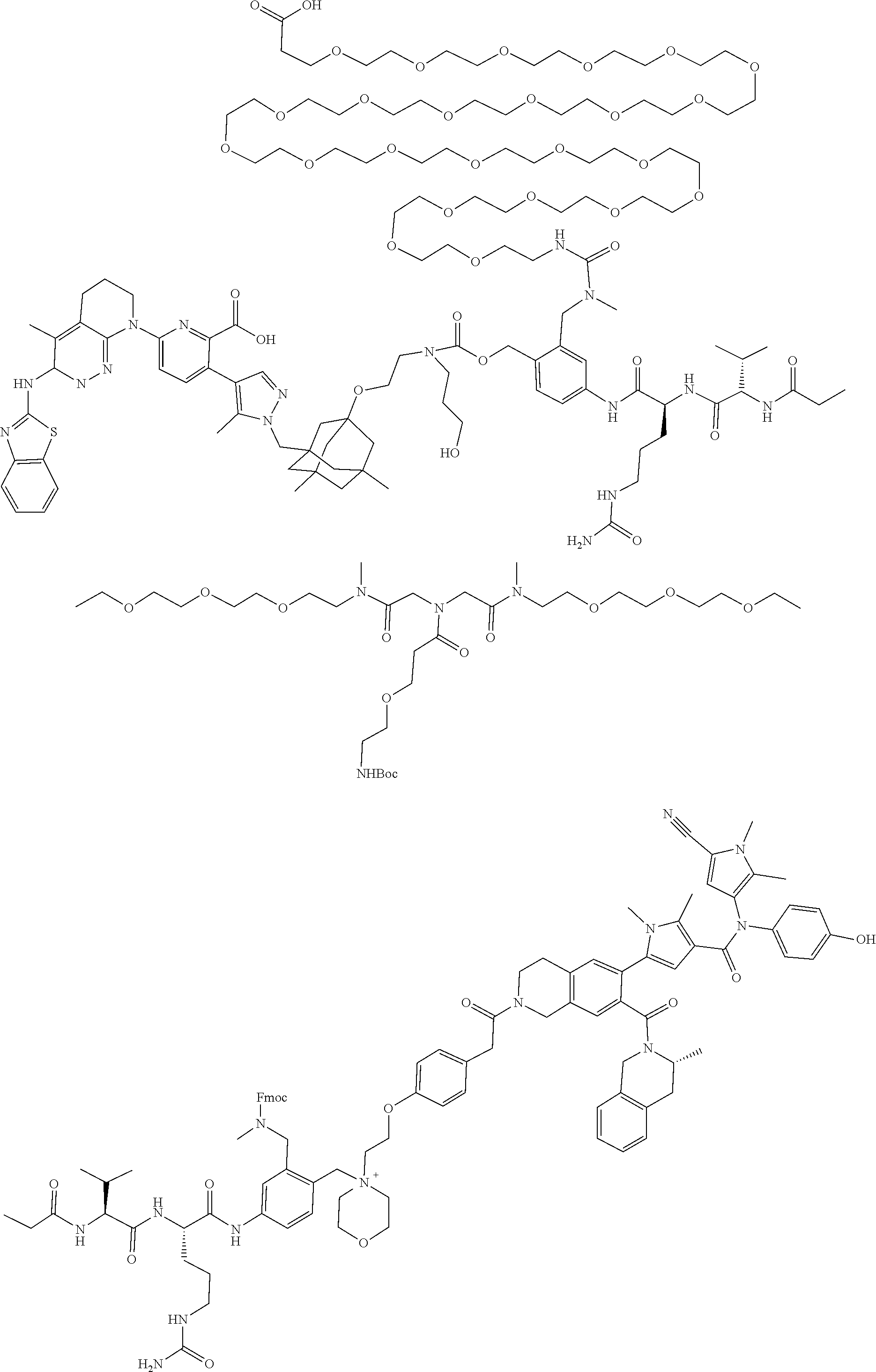

Following GENERAL PROCEDURE #4 using 4-(2-(((((9H-fluoren-9-yl)methoxy)carbonyl)(methyl)amino)methyl)-4-((32S,35S)-15-(3-(2-((tert-butoxycarbonyl)amino)ethoxy)propanoyl)-1-carboxy-32-isopropyl-12,18-dimethyl-13,17,30,33-tetraoxo-35-(3-ureidopropyl)-3,6,9,21,24,27-hexaoxa-12,15,18,31,34-pentaazahexatriacontan-36-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (181 mg, 75.2 μmol), TSTU (22.6 mg, 75.2 μmol) and DIEA (45.9 μL, 263.2 μmol), followed by 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid formate salt (184.9 mg, 75.2 μmol) and DIEA (45.9 μL, 263.2 μmol), 4-(2-(((((9H-fluoren-9-yl)methoxy)carbonyl)(methyl)amino)methyl)-4-((6S,9S,43S,46S)-1-amino-6-((4-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate (204 mg, 56% yield) was obtained. HRMS: M⁺=4684.4800, Rt=2.78 min (5 min acidic method).

### Synthesis of 4-(4-((6S,9S,43S,46S)-1-amino-6-((4-((((2-(((ls,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2

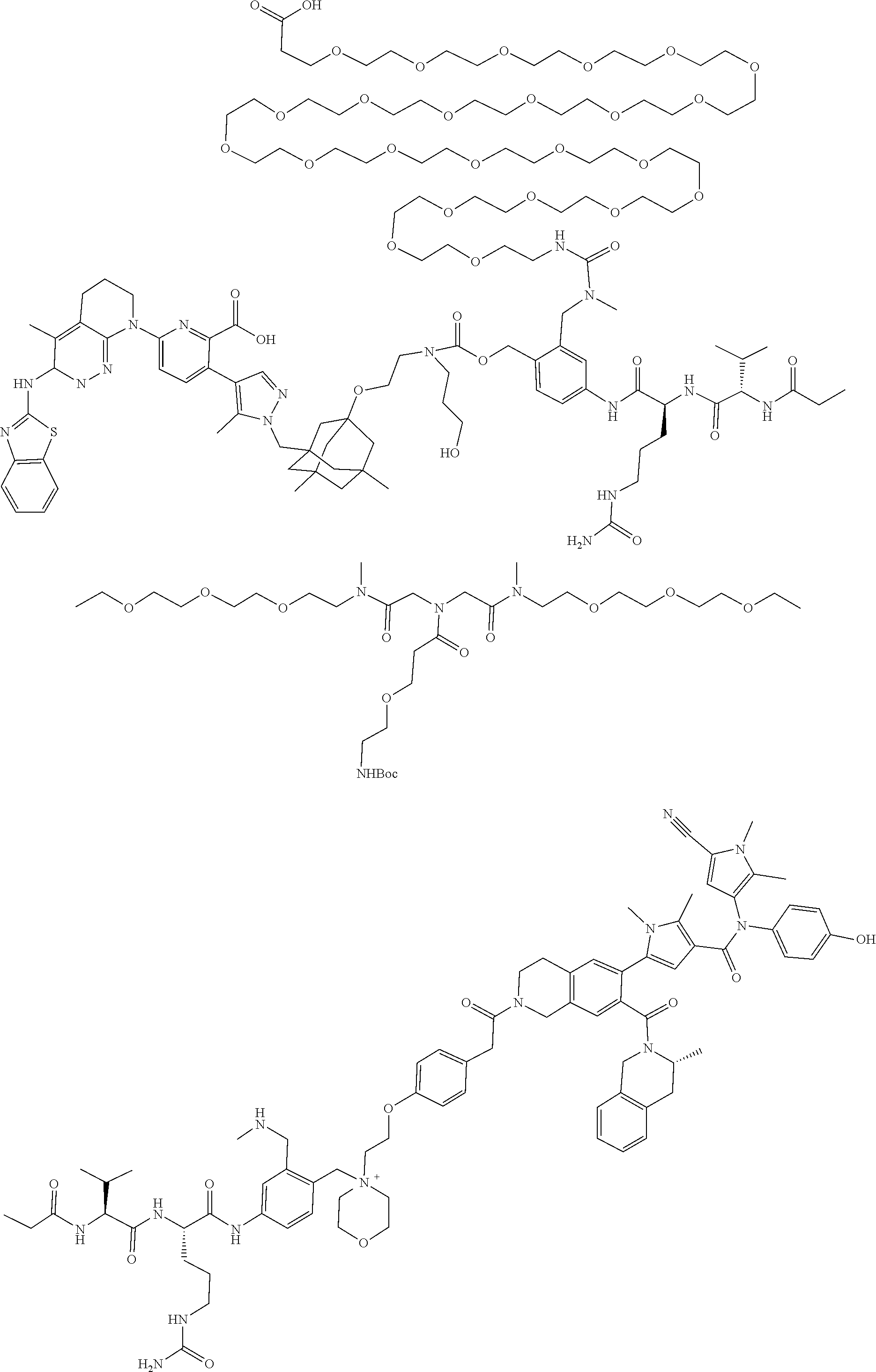

A solution of 4-(2-(((((9H-fluoren-9-yl)methoxy)carbonyl)(methyl)amino)methyl)-4-((6S,9S,43S,46S)-1-amino-6-((4-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate (204 mg, 43.1 μmol) in DMF was treated with piperidine (42.6 p L, 431 μmol) for 1 hour at which time the solution was diluted with DMSO and purified by ISCO RP-HPLC. Upon lyophilization, 4-(4-((6S,9S,43S,46S)-1-amino-6-((4-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate (104.4 mg, 55% yield) was obtained. HRMS: M⁺=4462.3999, Rt=2.69 min (5 min acidic method).

### Synthesis of 4-(4-((6S,9S,43S,46S)-1-amino-6-((4-((((2-(((ls,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate

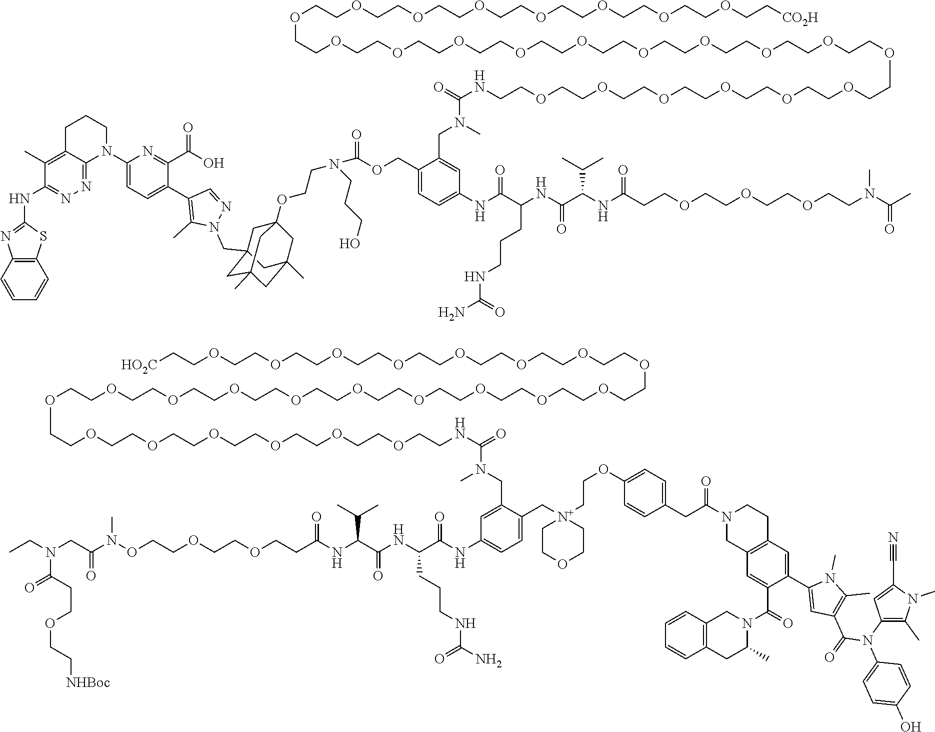

Following GENERAL PROCEDURE #1 using bis(4-nitrophenyl) carbonate (10.1 mg, 33.2 μmol), DIEA (18.2 μL, 104.7 μmol), 1-amino-3,6,9,12,15,18,21,24,27,30,33,36,39,42,45,48,51,54,57,60,63,66,69,72-tetracosaoxapentaheptacontan-75-oic acid (40 mg, 34.5 μmol), and then 4-(4-((6S,9S,43S,46S)-1-amino-6-((4-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate (110.2 mg, 24.4 μmol) and DIEA (36.4 p L, 209.4 μmol), 4-(4-((6S,9S,43S,46S)-1-amino-6-((4-((((2-(((1s,3r,5R,7S)-3-((4-(6-(3-(benzo[ld]thiazol-2-ylamino)-4-methyl-6,7-dihydropyrido[l2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate (99 mg, 68% yield) was obtained. HRMS: M⁺=5634.0298, Rt=2.38 min (5 min acidic method).

### Synthesis of 4-(4-((6S,9S,43S,46S)-1-amino-6-((4-((((2-(((1s,3r,5R,7S)-3-((4-(6-(3-(benzo[d]thiazol-2- ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate

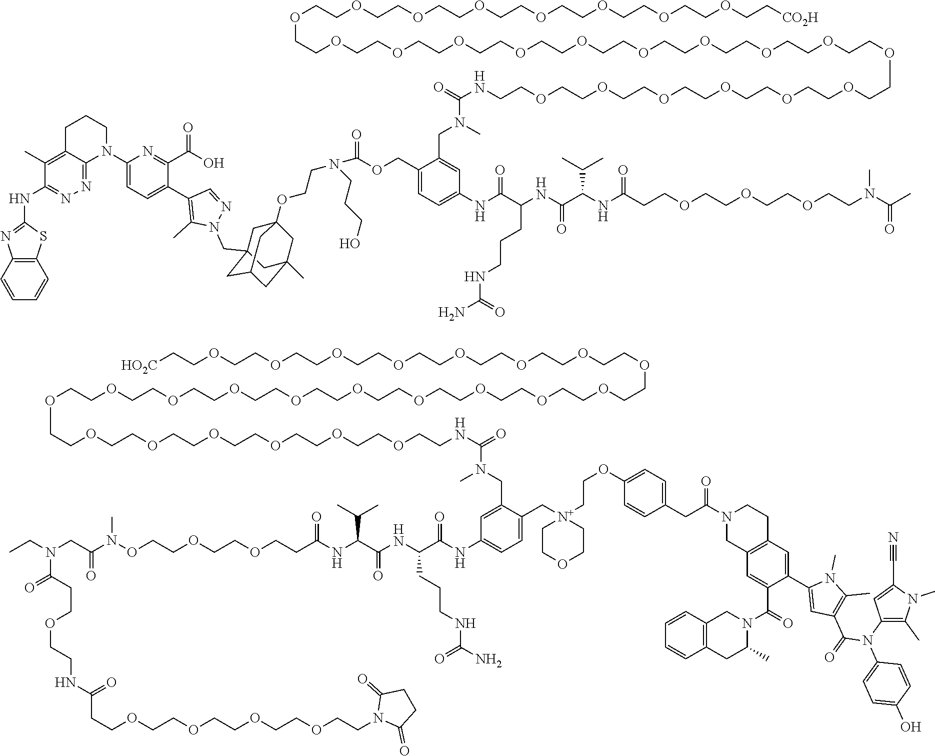

4-(4-((6S,9S,43S,46S)-1-amino-6-((4-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(3-(2-((tert-butoxycarbonyl)amino)ethoxy)propanoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate (99 mg, 17.4 μmol) was treated with 8 ml of 25% TFA/CH2Cl2 with 1% Et₃SiH for one hour, and after removal of volatiles in vacuo the residue was triturated with Et20. The material obtained was dissolved in DMF (3 mL) and this solution was added to 2M Me₂NH in MeOH (261 μL, 522.5 μmol) to clip the deBoc amine trifluoroacetyl ester byproduct that had formed in addition to deBoc amine, hydroxyl alkyl product. After standing for 30 minutes, the solution was diluted with DMSO and after purification by RP-HPLC with 0.05% formic acid modifier the deprotected amine hydroxy alkyl intermediate (47 mg, 47% yield) was isolated. HRMS: M⁺=5534.000, Rt=2.15 min (5 min acidic method). Following the second part of GENERAL PROCEDURE #5 and using isolated amine formate salt (47 mg, 8.4 μmol), 2,5-dioxopyrrolidin-1-yl 1-(2,5-dioxo-2,5-dihydro-1H-pyrrol-1-yl)-3,6,9,12-tetraoxapentadecan-15-oate (7.45 mg, 16.8 μmol) and DIEA (11.7 μL, 67.3 μmol), 4-(4-((6S,9S,43S,46S)-1-amino-6-((4-((((2-(((1s,3r,5R,7S)-3-((4-(6-(3-(benzo[d]thiazol-2-ylamino)-4-methyl-6,7-dihydropyrido[l2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)phenyl)carbamoyl)-26-(1-(2,5-dioxo-2,5-dihydro-1H-pyrrol-1-yl)-15-oxo-3,6,9,12,19-pentaoxa-16-azadocosan-22-oyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium formate (36 mg, 69% yield) was obtained. HRMS: M⁺=5861.1299, Rt=2.38 min (5 min acidic method).

### Synthesis of di-tert-butyl (2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl) phosphate

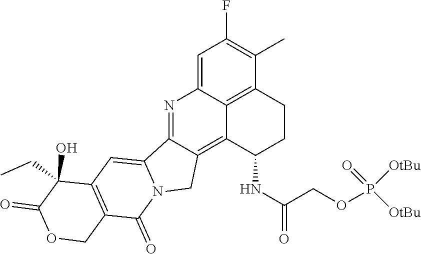 [SMILES: CC[C@@]1(O)C(=O)OCc2c1cc1n(c2=O)Cc2c-1nc1cc(F)c(C)c3c1c2[C@@H](NC(=O)COP(=O)(OC(C)(C)C)OC(C)(C)C)CC3]

To a mixture of (1S,9S)-9-ethyl-5-fluoro-9-hydroxy-4-methyl-10,13-dioxo-2,3,9,10,13,15-hexahydro-1H,12H-benzo[lde]pyrano[l3′,4′:6,7]indolizino[1l,2-b]quinolin-1-aminium methanesulfonate (420 mg, 790.2 μmol) and 2-((di-tert-butoxyphosphoryl)oxy)acetic acid (530 mg, 1976 μmol) in DMF (12 mL) was added TSTU (594.6 mg, 1976 μmol) and DIEA (1.39 mL, 7900 mmol). After stirring for 2 hours the solution was diluted with DMSO and purified by ISCO RP-HPLC. Upon lyophilization, di-tert-butyl (2-(((1S,9S)-9-ethyl-5-fluoro-9-hydroxy-4-methyl-10,13-dioxo-2,3,9,10,13,15-hexahydro-1H,12H-benzo[lde]pyrano[l3′,4′:6,7]indolizino[1l,2-b]quinolin-1-yl)amino)-2-oxoethyl) phosphate (308.3 mg, 57% yield) was obtained. LC/MS: (M-tBu-tBu+H⁺)+=574.4, Rt=1.05 min (2 min acidic method).

### Synthesis of 2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl dihydrogen phosphate

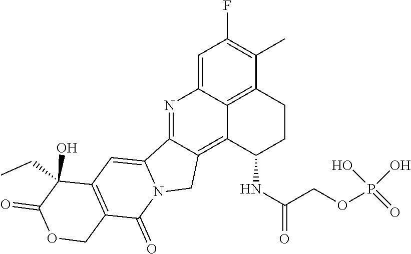 [SMILES: CC[C@@]1(O)C(=O)OCc2c1cc1n(c2=O)Cc2c-1nc1cc(F)c(C)c3c1c2[C@@H](NC(=O)COP(=O)(O)O)CC3]

di-tert-butyl (2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl) phosphate (308.3 mg, 449.6 μmol) was treated 25% TFA/CH2Cl2 (6 mL) at 0 C for 15 minutes and then at rt for 30 minutes at which time the volatiles were removed in vacuo. The residue was dissolved in DMSO and purified by ISCO RP-HPLC. Upon lyophilization, 2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl dihydrogen phosphate (188 mg, 73% yield) was obtained. LC/MS: MH⁺=574.4, Rt=0.53 min (2 min acidic method).

### Synthesis of allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-((methylamino)methyl)phenyl)amino)-1-oxo-5-ureidopentan-2-yl)amino)-3-methyl-1-oxobutan-2-yl)carbamate

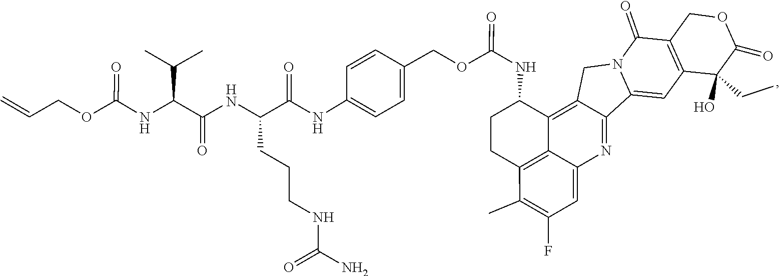 [SMILES: C=CCOC(=O)N[C@H](C(=O)N[C@@H](CCCNC(N)=O)C(=O)Nc1ccc(COC(=O)N[C@H]2CCc3c(C)c(F)cc4nc5c(c2c34)Cn2c-5cc3c(c2=O)COC(=O)[C@]3(O)CC)cc1)C(C)C]

A solution of 2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl dihydrogen phosphate (256 mg, 446.4 μmol) in DMF (1.5 mL) and N,N′-dicyclohexylmorpholine-4-carboximidamide (393 mg, 1339 μmol) was stirred for 15 minutes at which time (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate (500 mg, 670 μmol) was added. After stirring for 20 hours Et2NH (924 μL, 8928 μmol) was added. After stirring an additional 30 minutes the solution was diluted with DMSO and purified by ISCO RP-HPLC. Upon lyophilization, allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-((methylamino)methyl)phenyl)amino)-1-oxo-5-ureidopentan-2-yl)amino)-3-methyl-1-oxobutan-2-yl)carbamate (158 mg, 33% yield) was obtained as the formic acid salt. HRMS: MH⁺=1062.4000, Rt=1.75 min (5 min acidic method).

### Synthesis of allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)carbamate

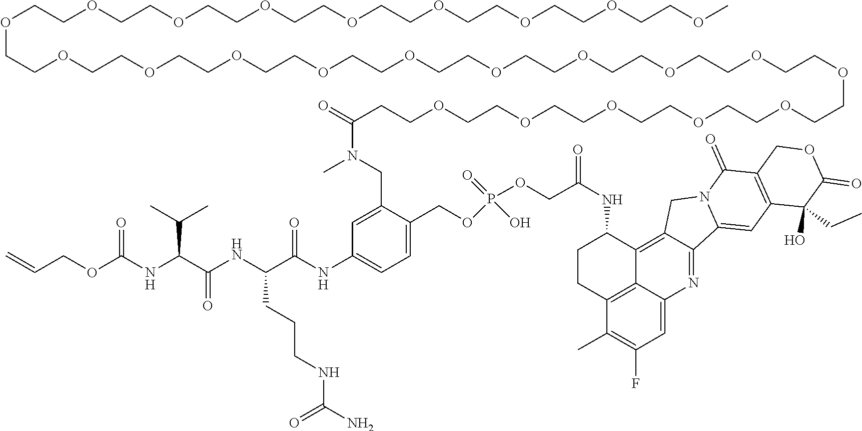 [SMILES: C=CCOC(=O)N[C@H](C(=O)N[C@@H](CCCNC(N)=O)C(=O)Nc1ccc(COP(=O)(O)OCC(=O)N[C@H]2CCc3c(C)c(F)cc4nc5c(c2c34)Cn2c-5cc3c(c2=O)COC(=O)[C@]3(O)CC)c(CN(C)C(=O)CCOCCOCCOCCOCCOCCOCCOCCOCCOCCOCCOCCOCCOCCOCCOCCOCCOCCOCCOCCOCCOCCOCCOCCOC)c1)C(C)C]

To a solution of 2,5,8,11,14,17,20,23,26,29,32,35,38,41,44,47,50,53,56,59,62,65,68,71-tetracosaoxatetraheptacontan-74-oic acid (433 mg, 387.5 μmol) in DMF (2 mL) was added HATU (433 mg, 387.5 μmol) followed by DIEA (338 μL, 1938 μmol). After stirring for 30 minutes a solution of allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-((methylamino)methyl)phenyl)amino)-1-oxo-5-ureidopentan-2-yl)amino)-3-methyl-1-oxobutan-2-yl)carbamate (343 mg, 323 μmol) in DMF (2 mL) was added followed by additional DIEA (338 μL, 1938 μmol). After stirring for 1 hour the solution was diluted with DMSO and purified by RP-HPLC. After lyophilization, allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)carbamate (155 mg, 22% yield) was obtained. HRMS: MH⁺=2161.0601, Rt=2.22 min (5 min acidic method).

### Synthesis of 4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl) hydrogen phosphate

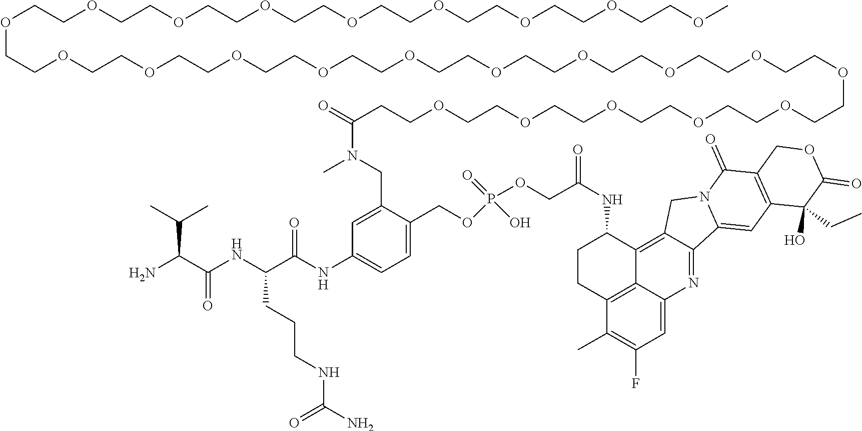 [SMILES: CC[C@@]1(O)C(=O)OCc2c1cc1n(c2=O)Cc2c-1nc1cc(F)c(C)c3c1c2[C@@H](NC(=O)COP(=O)(O)OCc1ccc(NC(=O)[C@H](CCCNC(N)=O)NC(=O)[C@@H](N)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC)CC3]

Following GENERAL PROCEDURE #2 using allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[lde]pyrano[l3′,4′:6,7]indolizino[l1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)amino)-3-methyl-1-oxobutan-2-yl)carbamate (155 mg, 71.6 μmol), phenyl silane (23.3 mg, 215 μmol), tetrakis(triphenylphosphine)palladium (0.83 mg, 0.72 μmol) in 1:1 CH₂Cl₂/MeOH (3 mL), 4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (2-(((1S,9S)-9-ethyl-5-fluoro-9-hydroxy-4-methyl-10,13-dioxo-2,3,9,10,13,15-hexahydro-1H,12H-benzo[lde]pyrano[l3′,4′:6,7]indolizino[11,2-b]quinolin-1-yl)amino)-2-oxoethyl) hydrogen phosphate (45 mg, 29% yield) was obtained as the TFA salt. HRMS: MH⁺=2077.0500, Rt=1.85 min (5 min acidic method).

### Synthesis of 16-(3-(2-((((9H-fluoren-9-yl)methoxy)carbonyl)amino)ethoxy)propanoyl)-13,19-dimethyl-14,18,31-trioxo-4,7,10,22,25,28,32-heptaoxa-13,16,19-triazapentatriacont-34-enoic acid

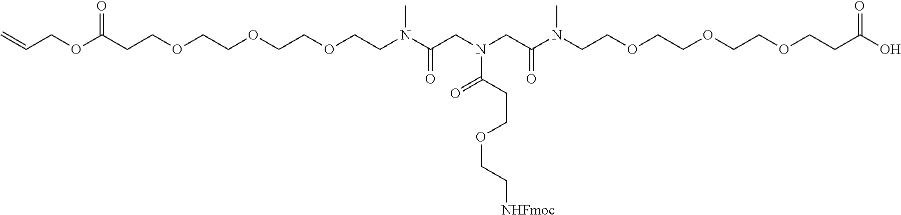

16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (114 mg, 138.5 p˜mol) was treated with 25% TFA/CH2Cl2 (4 mL) for one hour at which the volatiles were removed in vacuo. The residue was dissolved in DMF (1 mL) and (9H-fluoren-9-yl)methyl (2,5-dioxopyrrolidin-1-yl) carbonate (56.1 mg, 166 μmol) was added followed by DIEA (241 μL, 1385 μmol). After stirring for 30 minutes the solution was diluted with DMSO and purified by RP-HPLC. After lyophilization, 16-(3-(2-((((9H-fluoren-9-yl)methoxy)carbonyl)amino)ethoxy)propanoyl)-13,19-dimethyl-14,18,31-trioxo-4,7,10,22,25,28,32-heptaoxa-13,16,19-triazapentatriacont-34-enoic acid (60.1 mg, 46% yield) was obtained. LC/MS: MH⁺=945.9, Rt=1.01 min (2 min acidic method).

### Synthesis of allyl (6S,9S)-26-(3-(2-((((9H-fluoren-9-yl)methoxy)carbonyl)amino)ethoxy)propanoyl)-1-amino-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oate

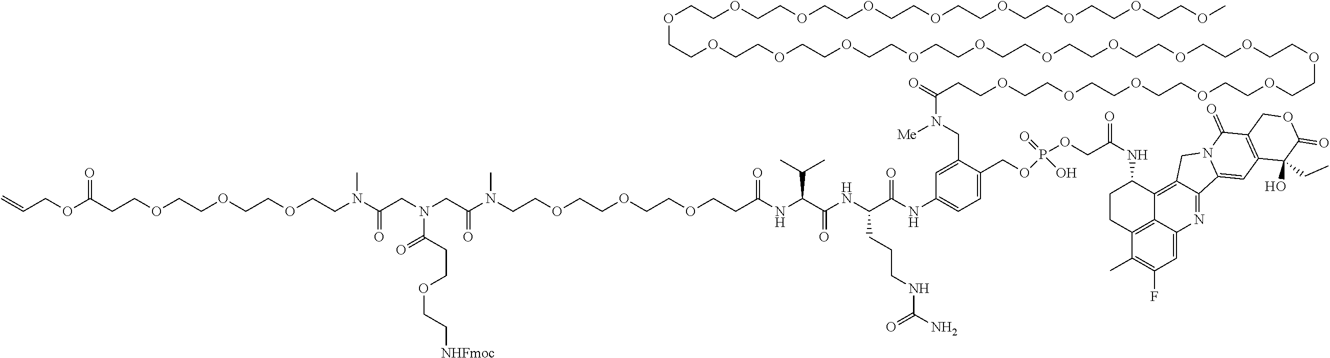

To a solution of 16-(3-(2-((((9H-fluoren-9-yl)methoxy)carbonyl)amino)ethoxy)propanoyl)-13,19-dimethyl-14,18,31-trioxo-4,7,10,22,25,28,32-heptaoxa-13,16,19-triazapentatriacont-34-enoic acid (27 mg, 28 μmol) in DMF (2 mL) was added TSTU (8.5 mg, 28.3 μmol) followed by DIEA (29.6 μL, 170 μmol). After stirring for 20 minutes a solution of 4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl) hydrogen phosphate TFA salt (58.8 mg, 28.3 μmol) in DMF (2 mL) was added followed by additional DIEA (29.6 μL, 170 μmol). After stirring for 3 hours the solution was diluted with DMSO and purified by RP-HPLC. After lyophilization, allyl (6S,9S)-26-(3-(2-((((9H-fluoren-9-yl)methoxy)carbonyl)amino)ethoxy)propanoyl)-1-amino-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oate (57 mg, 67% yield) was obtained. HRMS: MH⁺=3003.4900, Rt=2.71 min (5 min acidic method).

### Synthesis of (6S,9S)-26-(3-(2-((((9H-fluoren-9-yl)methoxy)carbonyl)amino)ethoxy)propanoyl)-1-amino-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid

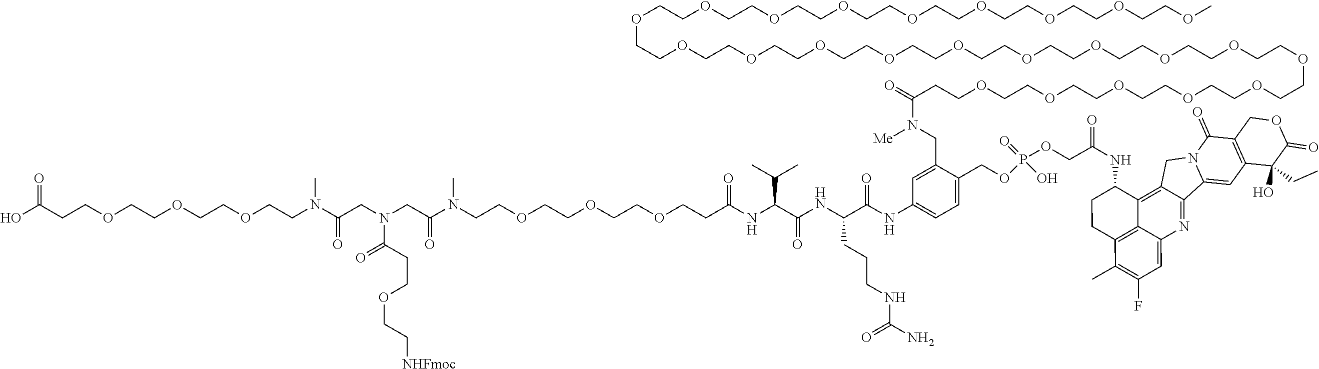

Following GENERAL PROCEDURE #2 using allyl (6S,9S)-26-(3-(2-((((9H-fluoren-9-yl)methoxy)carbonyl)amino)ethoxy)propanoyl)-1-amino-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oate (57 mg, 19 μmol), phenyl silane (6.2 mg, 56.9 μmol) and tetrakis(triphenylphosphine)palladium (ca. 1 mg, ca. 1 μmol) in 1:1 CH₂Cl₂/MeOH (3 mL), (6S,9S)-26-(3-(2-((((9H-fluoren-9-yl)methoxy)carbonyl)amino)ethoxy)propanoyl)-1-amino-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid (33.8 mg, 60% yield) was obtained. HRMS: MH⁺=2963.4600, Rt=2.50 min (5 min acidic method).

### Synthesis of 3-(1-(((1r,3s,5R,7S)-3-(2-((((4-((6S,9S,43S,46S)-26-(3-(2-((((9H-fluoren-9- yl)methoxy)carbonyl)amino)ethoxy)propanoyl)-1-amino-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid

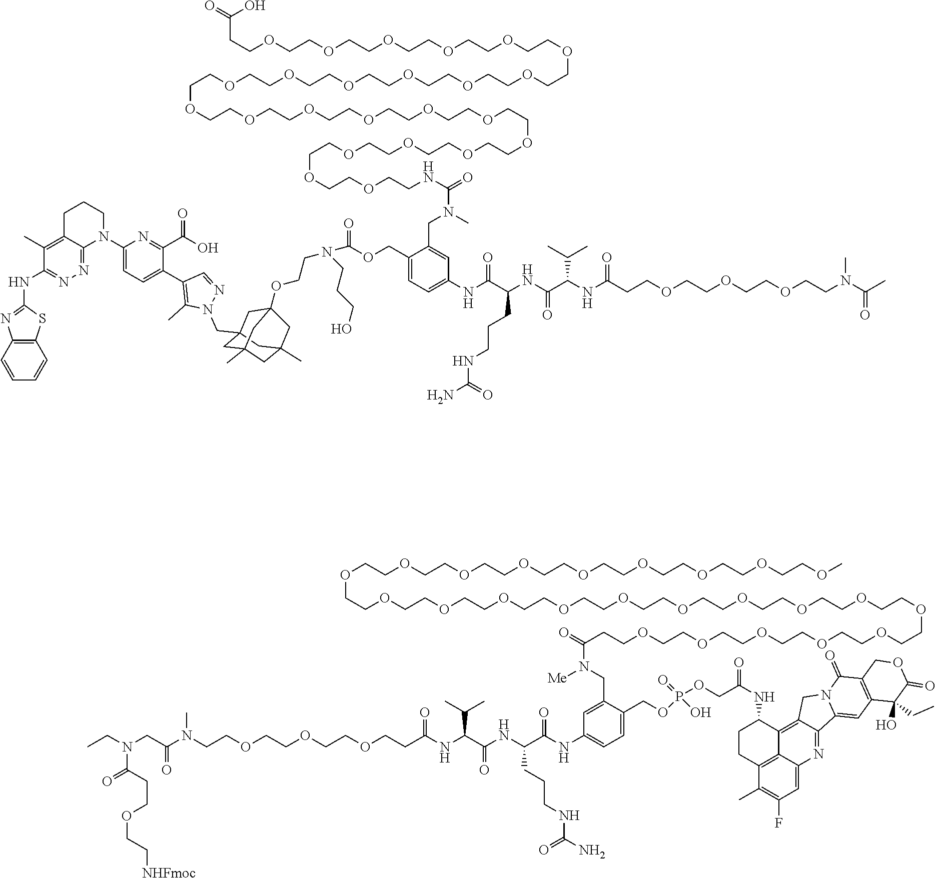

To a solution of (6S,9S)-26-(3-(2-((((9H-fluoren-9-yl)methoxy)carbonyl)amino)ethoxy)propanoyl)-1-amino-6-((4-((((2-(((1 S,9S)-9-ethyl-5-fluoro-9-hydroxy-4-methyl-10,13-dioxo-2,3,9,10,13,15-hexahydro-1 H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid (32.6 mg, 10.8 μmol) in DMF (1 mL) was added TSTU (3.3 mg, 10.8 μmol) followed by DIEA (9.4 μL, 54.1 μmol). After stirring for 30 minutes formic acid salt of 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,11,14,17,22,225,28,31,34,37,40,43,4,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (28.7 mg, 11.9 μmol) was added. After stirring for an additional 30 minutes the solution was diluted with DMSO and purified by RP-HPLC. After lyophilization, 3-(1-(((1r,3s,5R,7S)-3-(2-((((4-((6S,9S,43S,46S)-26-(3-(2-((((9H-fluoren-9-yl)methoxy)carbonyl)amino)ethoxy)propanoyl)-1-amino-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (31.9 mg, 55% yield) was obtained. HRMS: MH⁺=5356.7598, Rt=2.56 min (5 min acidic method).

### Synthesis of 3-(1-(((1r,3s,5R,7S)-3-(2-((((4-((6S,9S,43S,46S)-1-amino-26-(3-(2-aminoethoxy)propanoyl)-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid

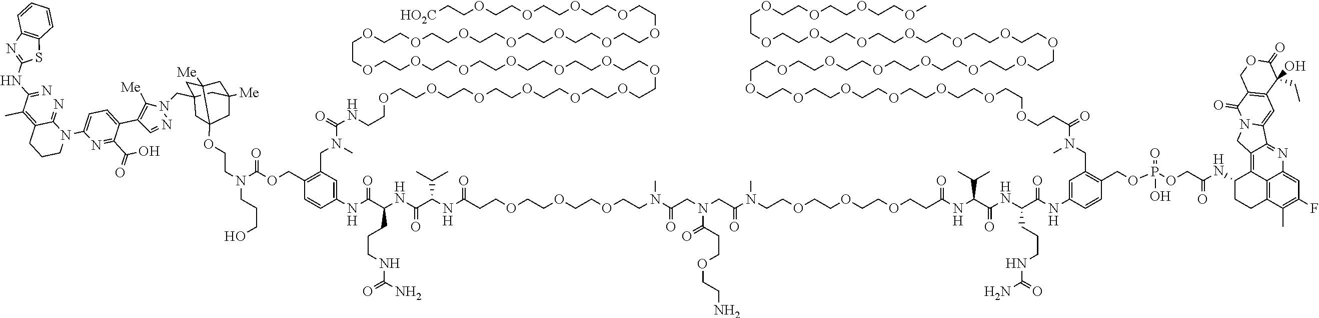

To a solution of 3-(1-(((1r,3s,5R,7S)-3-(2-((((4-((6S,9S,43S,46S)-26-(3-(2-((((9H-fluoren-9- yl)methoxy)carbonyl)amino)ethoxy)propanoyl)-1-amino-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (31.9 mg, 5.95 μmol) in DMF (0.8 mL) was added Et2NH (12.3 μL, 119 μmol). After stirring for 1 hour, the volatiles were removed in vacuo, the residue was dissolved in DMSO and was purified by ISCO RP-HPLC. Upon lyophilization, 3-(1-(((1r,3s,5R,7S)-3-(2-((((4-((6S,9S,43S,46S)-1-amino-26-(3-(2-aminoethoxy)propanoyl)-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (22.5 mg, 73% yield) was obtained as the formic acid salt. HRMS: MH⁺=5134.6899, Rt=2.22 min (5 min acidic method).

### Synthesis of 3-(1-(((1r,3s,5R,7S)-3-(2-((((4-((6S,9S,43S,46S)-1-amino-26-(1-(2,5-dioxo-2,5-dihydro-1H-pyrrol-1-yl)-15-oxo-3,6,9,12,19-pentaoxa-16-azadocosan-22-oyl)-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid

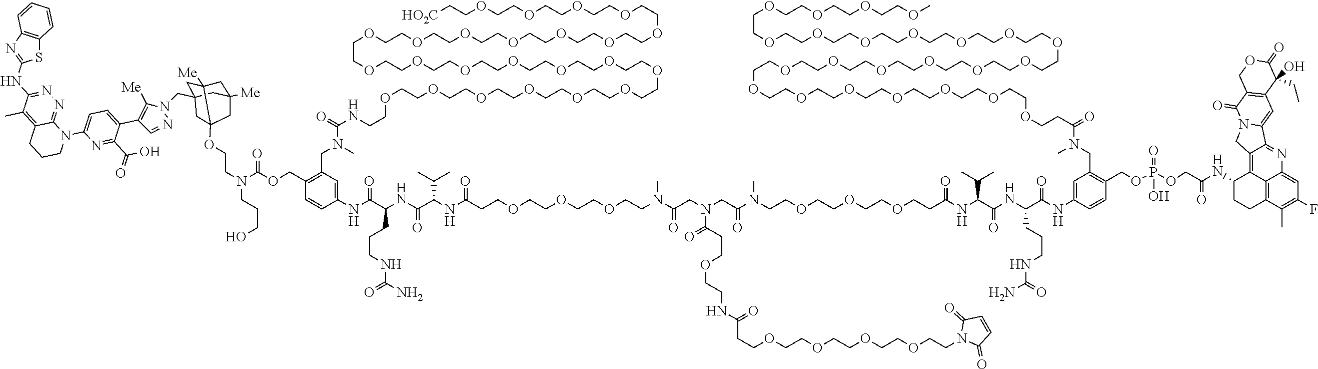

Following the second part of GENERAL PROCEDURE #5 and using 3-(1-(((1r,3s,5R,7S)-3-(2-((((4-((6S,9S,43S,46S)-1-amino-26-(3-(2-aminoethoxy)propanoyl)-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid formic acid salt (22.5 mg, 4.4 μmol), 2,5-dioxopyrrolidin-1-yl 1-(2,5-dioxo-2,5-dihydro-1H-pyrrol-l-yl)-3,6,9,12-tetraoxapentadecan-15-oate (3.9 mg, 8.8 μmol) and DIEA (6.1 p L, 35.1 μmol), 3-(1-(((1r,3s,5R,7S)-3-(2-((((4-((6S,9S,43S,46S)-1-amino-26-(1-(2,5-dioxo-2,5- dihydro-1H-pyrrol-l-yl)-15-oxo-3,6,9,12,19-pentaoxa-16-azadocosan-22-oyl)-6-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (17.1 mg, 71% yield) was obtained. HRMS: MH⁺=5461.8198, Rt=2.37 min (5 min acidic method). Linker-payload P5-L12-P8 was prepared using procedures similar to those described for P5-L37-P8.

### Synthesis of 4-(4-((S)-2-((S)-2-((tert-butoxycarbonyl)amino)-3-methylbutanamido)-5-ureidopentanamido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

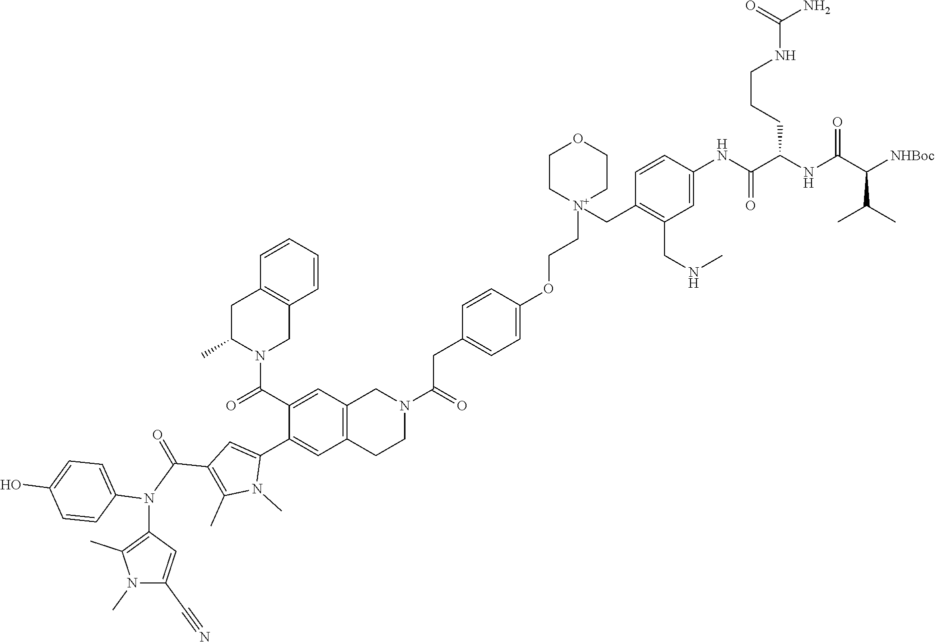 [SMILES: CNCc1cc(NC(=O)[C@H](CCCNC(N)=O)NC(=O)[C@@H](NC(=O)OC(C)(C)C)C(C)C)ccc1C[N+]1(CCOc2ccc(CC(=O)N3CCc4cc(-c5cc(C(=O)N(c6ccc(O)cc6)c6cc(C#N)n(C)c6C)c(C)n5C)c(C(=O)N5Cc6ccccc6C[C@H]5C)cc4C3)cc2)CCOCC1]

A solution of (R)-N-(4-((tert-butyldimethylsilyl)oxy)phenyl)-N-(5-cyano-1,2-dimethyl-1H-pyrrol-3-yl)-1,2-dimethyl-5-(7-(3-methyl-1,2,3,4-tetrahydroisoquinoline-2-carbonyl)-2-(2-(4-(2-morpholinoethoxy)phenyl)acetyl)-1,2,3,4-tetrahydroisoquinolin-6-yl)-1H-pyrrole-3-carboxamide (150 mg, 148 μmol), (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate (145 mg, 192 μmol), tetrabutylammonium iodide (21.9 mg, 59 μmol) and DIEA (26 p L, 148 μmol) in DMSO (0.6 mL) was stirred at rt for 40 hours. The solution was purified by ISCO C18 RP-HPLC. After lyophilization, the residue was dissolved in DMF (2 mL) and 2M dimethyl amine in MeOH (0.37 mL, ca. 740 μmol) was added. After standing two hours, 1.0 M tetrabutyl ammonium fluoride (0.2 mL, 200 μmol) was added. After 30 minutes, the solution was diluted in DMSO and purified by C18 ISCO RP-HPLC. Upon lyophilization, 4-(4-((S)-2-((S)-2-((tert-butoxycarbonyl)amino)-3-methylbutanamido)-5-ureidopentanamido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (185 mg, 76%) was obtained. HRMS: M⁺=1404.7500, Rt=1.75 min (5 min acidic method).

### Synthesis of 4-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

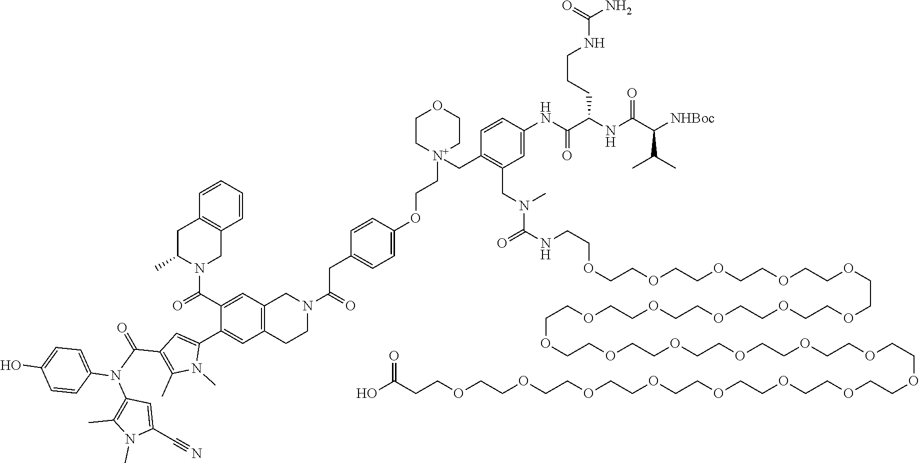 [SMILES: Cc1c(N(C(=O)c2cc(-c3cc4c(cc3C(=O)N3Cc5ccccc5C[C@H]3C)CN(C(=O)Cc3ccc(OCC[N+]5(Cc6ccc(NC(=O)[C@H](CCCNC(N)=O)NC(=O)[C@@H](NC(=O)OC(C)(C)C)C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CCOCC5)cc3)CC4)n(C)c2C)c2ccc(O)cc2)cc(C#N)n1C]

Following GENERAL PROCEDURE #1 using bis(4-nitrophenyl) carbonate (36.2 mg, 119 μmol), DIEA (59 μL, 340 μmol), 1-amino-3,6,9,12,15,18,21,24,27,30,33,36,39,42,45,48,51,54,57,60,63,66,69,72-tetracosaoxapentaheptacontan-75-oic acid (136 mg, 119 μmol), and then 4-(4-((S)-2-((S)-2-((tert-butoxycarbonyl)amino)-3-methylbutanamido)-5-ureidopentanamido)-2-((methylamino)methyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (185 mg, 113 μmol) and DIEA (59 μL, 340 μmol), 4-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (140 mg, 41% yield) was obtained. HRMS: M⁺=2576.4199, Rt=2.24 min (5 min acidic method).

### Synthesis of 4-(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate

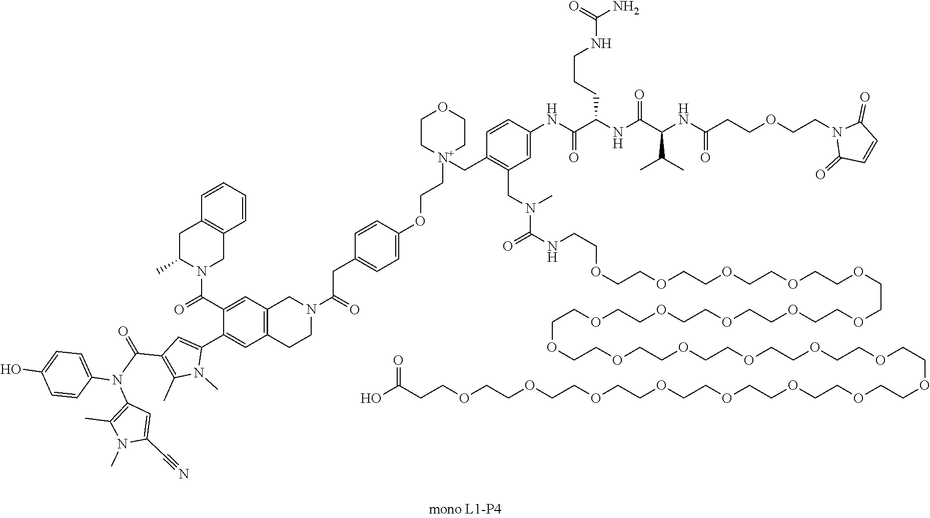 [SMILES: Cc1c(N(C(=O)c2cc(-c3cc4c(cc3C(=O)N3Cc5ccccc5C[C@H]3C)CN(C(=O)Cc3ccc(OCC[N+]5(Cc6ccc(NC(=O)[C@H](CCCNC(N)=O)NC(=O)[C@@H](NC(=O)CCOCCN7C(=O)C=CC7=O)C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CCOCC5)cc3)CC4)n(C)c2C)c2ccc(O)cc2)cc(C#N)n1C]

Following the first part of GENERAL PROCEDURE #5 and using 4-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (140 mg, 52 μmol), 69 mg of deprotected amine (49% yield) was obtained after RP-HPLC purification and lyophilization HRMS: M⁺=2476.3601, Rt=1.92 min (5 min acidic method). Following the second part of GENERAL PROCEDURE #5 and using deprotected amine (42.2 mg, 16 μmol), 2,5-dioxopyrrolidin-1-yl 3-(2-(2,5-dioxo-2,5-dihydro-1H-pyrrol-1-yl)ethoxy)propanoate (9.68 mg, 31 μmol) and DIEA (27 p L, 156 μmol), 4-(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-4-(2-(4-(2-(6-(4-((5-cyano-1,2-dimethyl-1H-pyrrol-3-yl)(4-hydroxyphenyl)carbamoyl)-1,5-dimethyl-1H-pyrrol-2-yl)-7-((R)-3-methyl-1,2,3,4-tetrahydroisoquinoline-2-carbonyl)-3,4-dihydroisoquinolin-2(1H)-yl)-2-oxoethyl)phenoxy)ethyl)morpholin-4-ium trifluoroacetate (37.2 mg, 85% yield) was obtained. HRMS: M⁺=2671.3999, Rt=2.21 min (5 min acidic method).

### Synthesis of 6-(3-(benzo[d]thiazol-2-ylamino)-4-methyl-6,7-dihydropyrido[2,3-c]pyridazin-8(5H)-yl)-3-(1-(((1r,3s,5R,7S)-3-(2-((((2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(3-hydroxypropyl)amino)ethoxy)-5,7-dimethyladamantan-1-yl)methyl)-5-methyl-1H-pyrazol-4-yl)picolinic acid

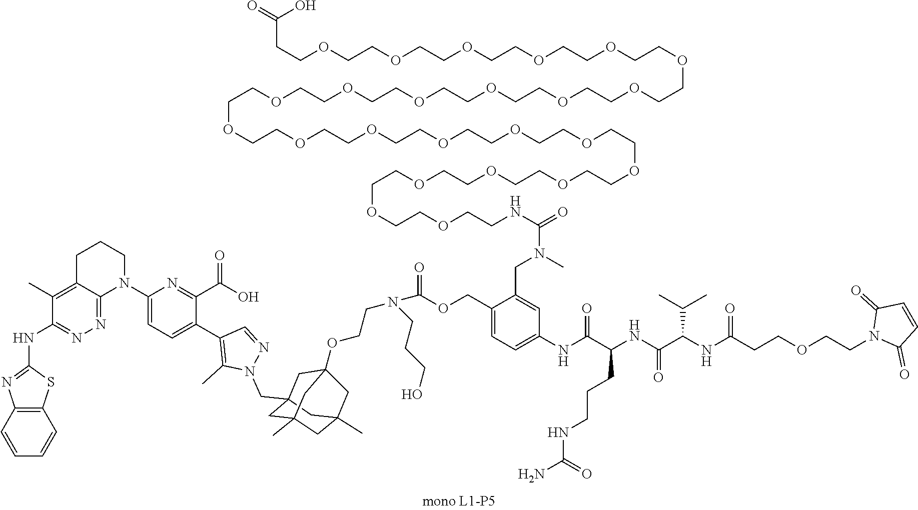 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Compound prepared as described within WO2022/115477.

### Synthesis of 6-(3-(benzo[d]thiazol-2-ylamino)-4-methyl-6,7-dihydropyrido[2,3-c]pyridazin-8(5H)-yl)-3-(1-(((1r,3s,5R,7S)-3-(2-((((2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(3-hydroxypropyl)amino)ethoxy)-5,7-dimethyladamantan-1-yl)methyl)-5-methyl-1H-pyrazol-4-yl)picolinic acid

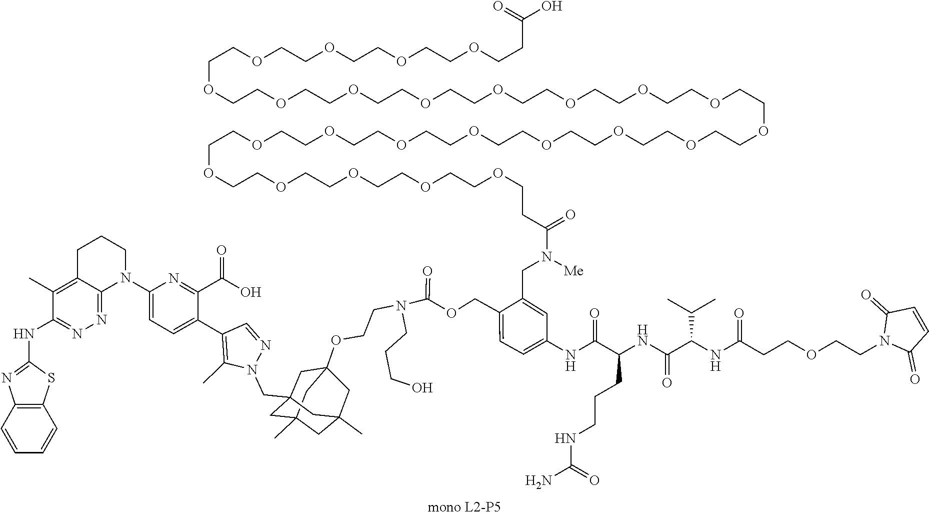 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Compound prepared as described within WO2022/115477.

### Synthesis of N-((S)-10-benzyl-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl)-6-(2,5-dioxo-2,5-dihydro-1H-pyrrol-1-yl)hexanamide

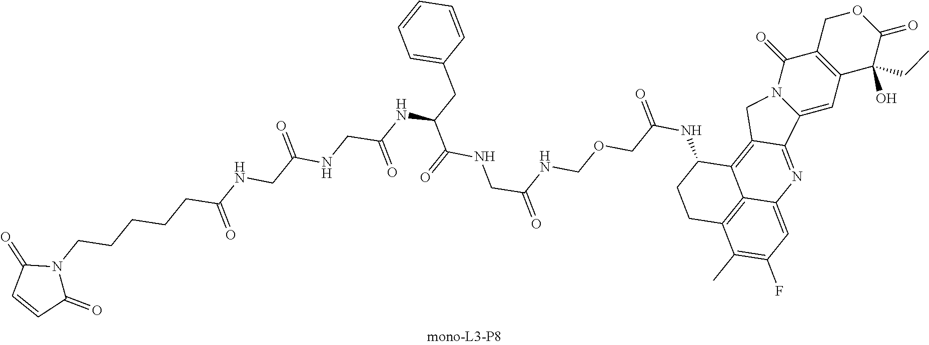 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

Compound, CAS #1599440-13-7, was obtained from commercial suppliers.

### Synthesis of 4-((S)-2-((S)-2-((tert-butoxycarbonyl)amino)-3-methylbutanamido)-5-ureidopentanamido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate

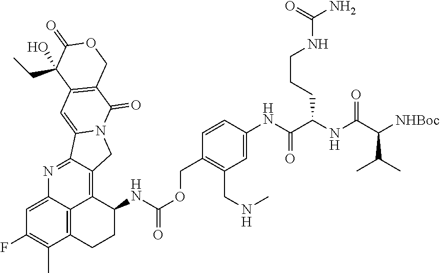 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)OC(C)(C)C)C(C)C)cc1CNC)CC3]

To a solution of (9H-fluoren-9-yl)methyl (5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (231 mg, 191.5 μmol) in DMF (4 mL) was added piperidine (189 μL, 1915 μmol). After standing for 30 minutes the solution was diluted with DMSO and purified by C18 ISCO RP-HPLC. Upon lyophilization 4-((S)-2-((S)-2-((tert-butoxycarbonyl)amino)-3-methylbutanamido)-5-ureidopentanamido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (133 mg, 63%) was obtained as the formic acid salt. HRMS: MH⁺=984.4800, Rt=1.85 min (5 min acidic method).

### Synthesis of 1-(5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid

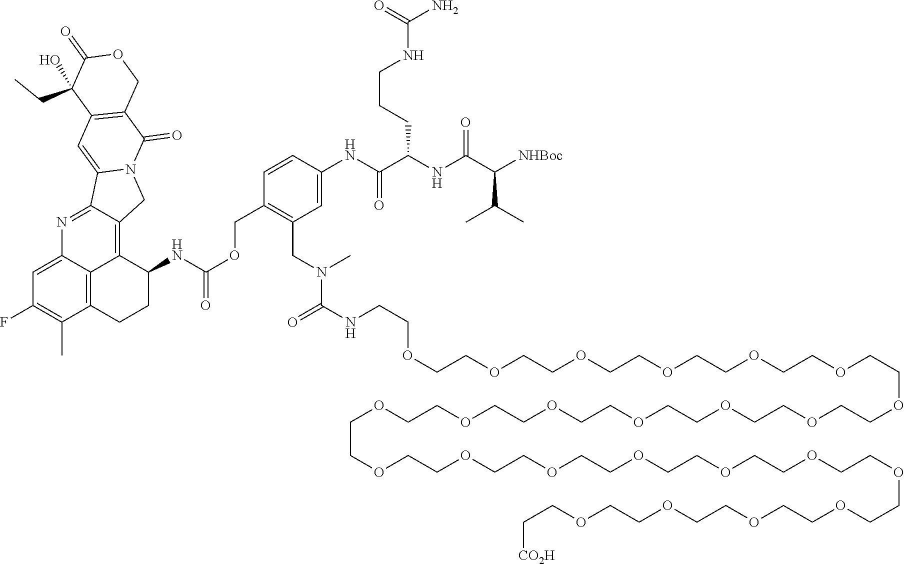 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)OC(C)(C)C)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3]

Following GENERAL PROCEDURE #1 using bis(4-nitrophenyl) carbonate (49.7 mg, 163.5 μmol), DIEA (94.9 μL, 545 μmol), 1-amino-3,6,9,12,15,18,21,24,27,30,33,36,39,42,45,48,51,54,57,60,63,66,69,72-tetracosaoxapentaheptacontan-75-oic acid (201.3 mg, 175.6 μmol), and then 4-((S)-2-((tert-butoxycarbonyl)amino)-5-ureidopentanamido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate formic acid salt (133 mg, 121.1 μmol) and DIEA (94.9 μL, 545 μmol), 1-(5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (200 mg, 76% yield) was obtained. HRMS: MH⁺=2156.1499, Rt=2.36 min (5 min acidic method).

### Synthesis of 1-(5-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid

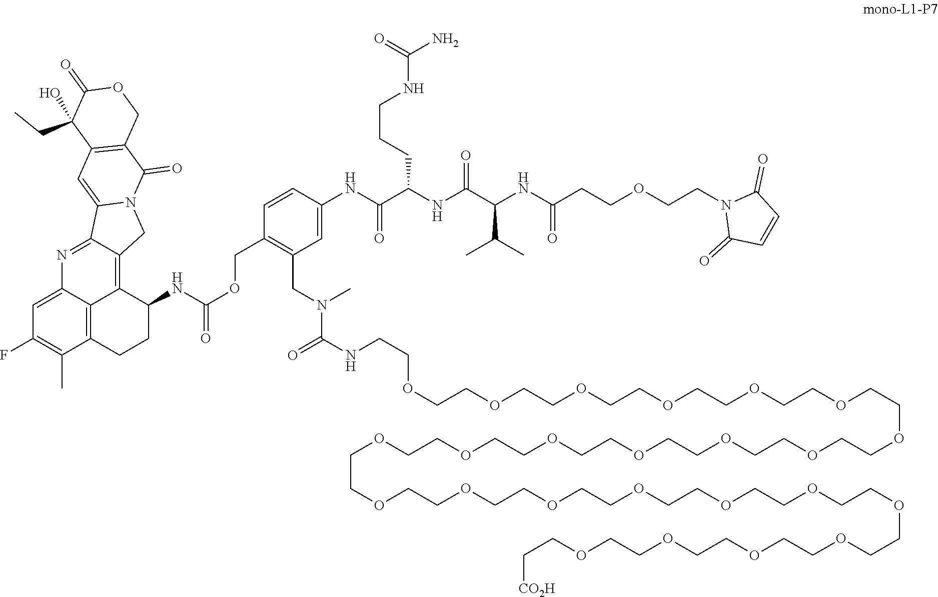 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3]

Following GENERAL PROCEDURE #5 and using 1-(5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (200 mg, 92.7 μmol), 2,5-dioxopyrrolidin-1-yl 3-(2-(2,5-dioxo-2,5-dihydro-1H-pyrrol-1-yl)ethoxy)propanoate (40.3 mg, 129.8 μmol) and DIEA (323 μL, 1855 μmol), 1-(5-((S)-2-((S)-2-(3-(2-(2,5-dioxo-2,5-dihydro-IH-pyrrol-1-yl)ethoxy)propanamido)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-i-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (67 mg, 32% yield) was obtained. HRMS: MH⁺=2251.1299, Rt=2.19 min (5 min acidic method).

### Example 3. Conjugation and Characterization of ADCs

In vitro Cysmab ADC preparation

Antibody (typically 5-10 mg) was incubated with rProtein A Sepharose resin (GE) at a ratio of 10 mg Ab to 1 ml resin in PBS for 15 minutes with mixing in an appropriately sized disposable column. Cysteine HCl was added to a final concentration of 20 mM and incubated with agitation for 30 min at room temperature to allow the reactive cysteines to be deblocked. The resin was rapidly washed with 50 column volumes PBS on a vacuum manifold in multiple additions. The resin was then resuspended in an equal volume PBS containing 250 nM CuCl₂. Reformation of antibody interchain disulfides was monitored by taking time points. At each time point, 25 μL of resin slurry was removed, 1 μL of 20 mM MC-valcit-MMAE was added, and the tube flicked several times. The resin was spun down, supernatant removed, and then eluted with 50 μL Antibody elution buffer (Thermo). The resin was pelleted and the supernatant analyzed by reverse phase chromatography using an Agilent PLRP-S 4000A 5 μm, 4.6×50 mm column (Buffer A is water, 0.1% TFA, Buffer B Acetonitrile, 0.1% TFA, column held at 80° C., Flowrate 1.5 ml/min; Gradient 0 minutes - 30% B, 5 minutes - 45% B, 6.5 min - 100% B, 8 minutes - 100% B, 10 minutes - 30%).

Once determined that the antibody has reformed its interchain disulfide bonds, the resin was washed with 10 column volumes PBS and the resin was resuspended in an equal volume PBS and 12 equivalents of the appropriate linker-payload (20 mM) in DMSO was added and then incubated at room temperature for 2 hours. The resin was then washed with 50 column volumes PBS to remove excess linker-payload. The ADC was eluted from the protein A resin with antibody elution buffer. The ADC was then dialyzed into PBS. The material was then concentrated using a centrifugal concentrator using an Amicon Ultra-15, 50KDa, regenerated cellulose (Millipore, UFC0905024), to 4.5 mg/ml and filtered sterilely through 0.22 μm sterile PVDF Filter, 25 mm (Millapore, SLGV013SL) and stored at 4° C. The following analyses were performed - analytical SEC to determine percent monomer, reduced mass spectroscopy to determine DAR, LAL test to determine endotoxin load and protein concentration was determined by A280 utilizing extinction coefficient and molecular weight of antibody. All in vitro materials were >90% monomer. Percent aggregation, as determined by comparison of the area of the high-molecular-weight peak absorbance at 210 and 280 nm with the area of the peak absorbance for monomeric ADC. HRMS data (protein method) indicated a dominant mass of the heavy chain+2 species, giving a DAR of ˜4.0 was calculated by comparing MS intensities of peaks for DAR1 DAR2 and DAR3 species.

General Methodology: Drug-to-antibody ratio (DAR) of exemplary ADCs was determined by liquid chromatography-mass spectrometry (LC/MS) according to the following method. For all LC methods, mobile phase A was purified MS grade water (Honeywell, LC015-1), mobile phase B was MS grade 80% Isopropanol (Honeywell LC323-1): 20% acetonitrile (Honeywell, LC015-1), LC323-1), supplemented with 1% of formic acid (FA) (Thermo Scientific, 85178). The column temperature was set at 80° C. A general MS method was optimized for all ADCs synthesized. The column used for analysis was an Agilent PLRP-S 4000 A; 2.1×150 mm, 8 um (Agilent, PL1912-3803). Flowrate used was 0.3 ml/min. The gradient used was 0-0.75 minute 95% A, 0.76-1.9 minute 75% A, 1.91-11.0 minute 50% A, 11.01-11.50 10% A, 11.51-13.50 minute 95% A, 13.51-18 minute 95% A on an Acuity Bio H-Class Quaternary UPLC (Waters). MS system was Xevo G2-XS QToF ESI mass spectrometer (Waters) and data acquired from 1.5-11 minutes and masses were analyzed between 15000-80000 daltons. DAR was determined from the deconvoluted spectra or UV chromatogram by summing the integrated MS (total ion current) or UV (280 nm) peak area of unconjugated and conjugated given species (mAb or associated fragment), weighted by multiplying each area by the number of drug attached. The summed, weighted areas were divided by the sum of total area and the results produced a final average DAR value for the full ADC.

Size exclusion chromatography (SEC): SEC was performed to determine the quality of the ADCs and aggregation percentage (%) after purification. The analysis was performed on analytical column Superdex 200 Increase 5/150 GL (GE Healthcare, 28990945) in isocratic conditions 100% PBS pH 7.2 ((Hyclone SH30028.03)), flow 0.45 ml/min for 8 minutes. The % aggregate fraction of the ADC sample was quantified based on the peak area absorbance at 280 nm. Calculation was based on the ratio between the high molecular weight eluent at 280 nm divided by the sum of peak area absorbance at the same wavelength of the high molecular weight and monomeric eluents multiplied by 100%. Data was acquired on an Agilent Bio-Inert 1260 HPLC outfitted with a Wyatt miniDAWN light scattering and Treos refractive index detectors (Wyatt Technologies, Santa Barbara, CA).

In vivo Cysmab ADC preparation

Antibody (25-200 mg) was incubated with rProtein A Sepharose resin (Cytiva) at a ratio of 10 mg Ab to 1 ml resin in PBS for 15 minutes with mixing in an appropriately sized disposable column. Cysteine HCl was added to a final concentration of 20 mM and incubated with agitation for 30 min at room temperature to allow the reactive cysteines to be deblocked. The resin was rapidly washed with 50 column volumes phosphate buffered saline pH 7.2 (PBS) on a vacuum manifold in multiple additions. The resin was then resuspended in an equal volume PBS containing 250 nM CuCl₂. Reformation of antibody interchain disulfides was monitored by taking time points. At each time point, 25 μL of resin slurry was removed, 1 μL of 20 mM MC-valcit-MMAE was added, and the tube flicked several times. The resin was spun down, supernatant removed, and then eluted with 50 μL Antibody elution buffer (Thermo). The resin was pelleted and the supernatant analyzed by reverse phase chromatography using an Agilent PLRP-S 4000A 5 μm, 4.6×50 mm column (Buffer A is water, 0.1% TFA, Buffer B Acetonitrile, 0.1 % TFA, column held at 80° C., Flowrate 1.5 ml/min; Gradient 0 minutes - 30% B, 5 minutes - 45% B, 6.5 min - 100% B, 8 minutes - 100% B, 10 minutes - 30%).

Once it was determined that the antibody has reformed its interchain disulfide bonds, the resin was washed with 10 column volumes PBS and the resin was resuspended in an equal volume PBS and 12 equivalents of the appropriate linker-payload (20 mM) in DMSO was added and then incubated at room temperature for 2 hours. The resin was then washed with 50 column volumes PBS to remove excess linker-payload. The ADC was eluted from the protein A resin with antibody elution buffer. The ADC was then dialyzed into PBS and preparative SEC using a 16/60 or 26/600 S200increase pg SEC column (GE) with PBS as the mobile phase if needed. The material was then concentrated using a centrifugal concentrator using an Amicon Ultra-15, 50KDa, regenerated cellulose (Millipore, UFC0905024), to 4.5 mg/ml and filtered sterilely through 0.22 μm sterile PVDF Filter, 25 mm (Millapore, SLGV013SL) and stored at 4° C. The following analyses were performed - analytical SEC to determine percent monomer, mass spectroscopy to determine DAR, LAL test to determine endotoxin load and protein concentration was determined by A280 utilizing extinction coefficient and molecular weight of antibody. All in vivo materials were >95% monomer. Aggregation was typically <10%. Percent aggregation, as determined by comparison of the area of the high-molecular-weight peak absorbance at 210 and 280 nm with the area of the peak absorbance for monomeric ADC. HRMS data (protein method) indicated a dominant mass of the heavy chain+2 species, giving a DAR of ˜4.0 was calculated by comparing MS intensities of peaks for DAR1 DAR2 and DAR3 species.

General Methodology: Drug-to-antibody ratio (DAR) of exemplary ADCs was determined by liquid chromatography-mass spectrometry (LC/MS) according to the following method. For all LC methods, mobile phase A was purified MS grade water (Honeywell, LC015-1), mobile phase B was MS grade 80% Isopropanol (Honeywell LC323-1): 20% acetonitrile (Honeywell, LC015-1), LC323-1), supplemented with 1% of formic acid (FA) (Thermo Scientific, 85178). The column temperature was set at 80° C. A general MS method was optimized for all ADCs synthesized. The column used for analysis was an Agilent PLRP-S 4000 A; 2.lxl50 mm, 8 um (Agilent, PL1912-3803). Flowrate used was 0.3 ml/min. The gradient used was 0-0.75 minute 95% A, 0.76-1.9 minute 75% A, 1.91-11.0 minute 50% A, 11.01-11.50 10% A, 11.51-13.50 minute 95% A, 13.51-18 minute 95% A on an Acuity Bio H-Class Quaternary UPLC (Waters). MS system was Xevo G2-XS QToF ESI mass spectrometer (Waters) and data acquired from 1.5-11 minutes and masses were analyzed between 15000-80000 daltons. DAR was determined from the deconvoluted spectra or UV chromatogram by summing the integrated MS (total ion current) or UV (280 nm) peak area of unconjugated and conjugated given species (mAb or associated fragment), weighted by multiplying each area by the number of drug attached. The summed, weighted areas were divided by the sum of total area and the results produced a final average DAR value for the full ADC.

Size exclusion chromatography (SEC): SEC was performed to determine the quality of the ADCs and aggregation percentage (%) after purification. The analysis was performed on analytical column Superdex 200 Increase 5/150 GL (GE Healthcare, 28990945) in isocratic conditions 100% PBS pH 7.2 ((Hyclone SH30028.03)), flow 0.45 ml/min for 8 minutes. The % aggregate fraction of the ADC sample was quantified based on the peak area absorbance at 280 nm. Calculation was based on the ratio between the high molecular weight eluent at 280 nm divided by the sum of peak area absorbance at the same wavelength of the high molecular weight and monomeric eluents multiplied by 100%. Data was acquired on an Agilent Bio-Inert 1260 HPLC outfitted with a Wyatt miniDAWN light scattering and Treos refractive index detectors (Wyatt Technologies, Santa Barbara, CA).

### Example 4. In Vitro Assessment CD48 MCL1 & BCL2 Dual ADCs

### Cell Lines

The CD48 MCL-li and BCL-2i antibody drug conjugates were tested against nine endogenous cancer cell lines.
KHM-1B: JCRB No. JCRB0133 cultured in RPMI-1640+20% FBS
AMO-1: DSMZ No. ACC-538 cultured in RPMI-1640+20% FBS
KMS-27: JCRB No. JCRB1188 cultured in RPMI-1640+10% FBS
KMS-21-BM: JCRB No. JCRB1185 cultured in RPMI-1640+10% FBS
NCI-H929: ATCC No. CRL-9068 cultured in RPMI-1640+10% FBS+0.05 mM 2-mercaptoethanol
RL: ATCC No. CRL-2261 cultured in RPMI-1640+10% FBS
WSU-DLCL2: DSMZ No. ACC-575 cultured in RPMI-1640+10% FBS
MOLP-8: DSMZ No. ACC-569 cultured in RPMI-1640+20% FBS
KE-97: RCB No. RCB1435 cultured in RPMI-1640+10% FBS
   Inhibition of cell proliferation and survival

The ability of the MCL-1 and BCL-2 antibody drug conjugates to inhibit cell proliferation and survival was assessed using the Promega CellTiter-Glo® proliferation assay.

Cell lines were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cell viability and cell density were determined using a cell counter (Vi-Cell XR Cell Viability Analyzer, Beckman Coulter). Cells with higher than 85% viability were seeded in white clear bottom 384-well TC treated plates (Corning cat. #3765). Cells were seeded at a density of 1,000 cells per well in 45 μL of standard growth media. Plates were incubated at 5% CO₂, 37° C. overnight in a tissue culture incubator. The next day, free MCL-1 and BCL-2 payloads, targeting MCL-1 and BCL-2 ADCs, and non-targeting isotype ADCs free MCL-1 and BCL-2 payloads, were prepared at 10× in standard growth media. The prepared treatments were added to the cells resulting in final concentrations of 0.013-250 nM and a final volume of 50 μL per well. Each drug concentration was tested in quadruplets. Plates were incubated at 5% CO₂, 37° C. for 5 days in a tissue culture incubator, after which cell viability was assessed through the addition of 25 L μof CellTiter Glo® (Promega, cat #G7573), a reagent which lyses cells and measures total adenosine triphosphate (ATP) content. Plates were incubated at room temperature for 10 minutes to stabilize luminescent signals prior to reading using a luminescence reader (EnVision Multilabel Plate Reader, PerkinElmer). To evaluate the effect of the drug treatments, luminescent counts from wells containing untreated cells (100% viability) were used to normalize treated samples. A variable slope model was applied to fit a nonlinear regression curve to the data in GraphPad PRISM version 7.02 software. IC50 and Amax values were extrapolated from the resultant curves.

The concentrations of treatment required to inhibit 50% of cell growth or survival (IC50) were calculated with representative IC50 values of the cell lines tested summarized in Table 1. The representative cancer cell lines were shown to be sensitive to the MCL-1 payload, P1, and the BCL-2 payloads, P2, P3 and P4. KHM-1B, KMS-27, KMS-21-BM, NCI-H929 and AMO-1 were most sensitive to the CD48 targeting MCL-1 and BCL-2 ADCs with IC50s ranging from 0.031-1.49 nM. The targeting ADCs NY920-P1-L17-P4 and NY920-P1-L12-P4 were the most potent ADCs tested. There was little to no in vitro activity observed from the isotype matched non-targeting control ADCs. These studies indicate that MCL-1 and BCL-2 ADCs were capable of inhibiting cell proliferation on various cancer cell lines expressing CD48.

TABLE 1
CD48 MCL-1 and BCL-2 ADC Cytotoxicity

| | KHM- | KMS- | KMS- | NCI- | | | WSU- | MOLP- | |
|---|---|---|---|---|---|---|---|---|---|
| | 1B | 27 | 21-BM | H929 | RL | AMO-1 | DLCL2 | 8 | Ke97 |
| | IC50 | IC50 | IC50 | IC50 | IC50 | IC50 | IC50 | IC50 | IC50 |
| Compound | (nM) | (nM) | (nM) | (nM) | (nM) | (nM) | (nM) | (nM) | (nM) |
| NY920-P1- | 0.037 | 0.305 | 0.380 | 0.217 | — | <5 | >50 | >50 | <1 |
| L12-P2 | | | | | | | | | |
| NY920-P1- | 0.037 | 0.216 | 0.146 | 0.060 | — | 0.407 | >50 | >10 | 0.160 |
| L19-P2 | | | | | | | | | |
| NY920-P1- | 0.033 | <1 | 0.185 | 0.174 | 221.8 | 1.07 | >50 | >10 | — |
| L12-P3 | | | | | | | | | |
| NY920-P1- | 0.046 | 0.257 | 0.320 | 0.121 | >250 | 0.832 | >50 | >5 | 0.287 |
| L17-P3 | | | | | | | | | |
| NY920-P1- | 0.031 | 0.041 | 0.330 | 0.133 | 23.6 | 1.49 | 1.48 | >5 | 1.11 |
| L17-P4 | | | | | | | | | |
| NY920-P1- | 0.036 | 0.011 | 0.254 | 0.070 | 19.67 | 0.531 | <50 | >10 | 0.027 |
| L12-P4 | | | | | | | | | |
| IgG-P1-L19-P2 | — | — | — | — | — | — | — | — | — |
| IgG-P1-L12-P4 | >50 | 3.05 | — | — | 20.47 | — | — | — | — |
| P4 | >10 | 0.999 | >100 | >250 | 1.26 | >250 | 4.17 | >250 | >250 |
| P3 | >10 | 3.91 | >100 | >250 | 11.8 | >250 | 5.41 | >250 | >250 |
| P2 | >10 | 0.316 | >100 | >250 | 6.26 | >250 | 3.77 | >250 | >250 |
| P1 | 1.62 | 13.3 | 1.66 | 5.17 | >250 | 4.70 | 41.1 | 6.17 | >250 |

### Example 5. In Vitro Assessment CD74 MCL1 & BCL2 Dual ADCs

### Cell Lines

The CD74 Milatuzumab DANAPA E152C S375C MCL-li and BCL2i antibody drug conjugates were tested against one endogenous cancer cell line.

EOL-1: DSMZ No. ACC-386 cultured in RPMI-1640+10% FBS

Inhibition of cell proliferation and survival

The ability of the MCL-1 and BCL-2 antibody drug conjugates to inhibit cell proliferation and survival was assessed using the Promega CellTiter-Glo* proliferation assay.

Cell lines were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cell viability and cell density were determined using a cell counter (Vi-Cell XR Cell Viability Analyzer, Beckman Coulter). Cells with higher than 85% viability were seeded in white clear bottom 384-well TC treated plates (Corning cat. #3765). Cells were seeded at a density of 1,000 cells per well in 45 L μof standard growth media. Plates were incubated at 5% CO₂, 37° C. overnight in a tissue culture incubator. The next day, free MCL-1 payload (P1) and targeting MCL-1 and BCL-2 ADCs were prepared at 10X in standard growth media. The prepared treatments were added to the cells resulting in final concentrations of 0.008-150 nM and a final volume of 50 μL per well. Each drug concentration was tested in quadruplets. Plates were incubated at 5% CO₂, 37° C. for 5 days in a tissue culture incubator, after which cell viability was assessed through the addition of 25 L μof CellTiter Glo® (Promega, cat #G7573), a reagent which lyses cells and measures total adenosine triphosphate (ATP) content. Plates were incubated at room temperature for 10 minutes to stabilize luminescent signals prior to reading using a luminescence reader (EnVision Multilabel Plate Reader, PerkinElmer). To evaluate the effect of the drug treatments, luminescent counts from wells containing untreated cells (100% viability) were used to normalize treated samples. A variable slope model was applied to fit a nonlinear regression curve to the data in GraphPad PRISM version 7.02 software. IC50 and Amax values were extrapolated from the resultant curves.

The concentrations of treatment required to inhibit 50% of cell growth or survival (IC50) were calculated with representative C50 values of the cell lines tested summarized in Table 2. The representative cancer cell line was shown to be sensitive to the MCL-1 payload, P1, with an IC50 value of 0.338 nM activity. The CD74 targeting MCL-1 and BCL-2 ADCs tested on EOL-1 demonstrated in vitro efficacy with IC50s ranging from 0.030-0.853 nM. These studies indicate that MCL-1 and BCL-2 ADCs were capable of inhibiting cell proliferation on a cancer cell line expressing CD74.

TABLE 2
CD74 Milatuzumab DANAPA E152C S375C
MCL-1i and BCL2i ADC Cytotoxicity

| Compound | IC50 (nM) | Amax (Span) |
|---|---|---|
| CD74 Milatuzumab DANAPA | 0.853 | 100.3 |
| E152C S375C-P1-L35-P1 | | |
| CD74 Milatuzumab DANAPA | 0.030 | 65.4 |
| E152C S375C-P1-L12-P4 | | |
| CD74 Milatuzumab DANAPA | 0.292 | 91.3 |
| E152C S375C-P1-L12-P3 | | |
| CD74 Milatuzumab DANAPA | 0.217 | 84.7 |
| E152C S375C-P2-L13-P1 | | |
| CD74 Milatuzumab DANAPA | 0.247 | 93.0 |
| E152C S375C-P1-L32-P2 | | |
| CD74 Milatuzumab DANAPA | 0.238 | 82.5 |
| E152C S375C-P1-L30-P2 | | |
| CD74 Milatuzumab DANAPA | 0.122 | 97.6 |
| E152C S375C-P1-L17-P2 | | |
| CD74 Milatuzumab DANAPA | 0.269 | 107.4 |
| E152C S375C-P1-L27-P1 | | |
| CD74 Milatuzumab DANAPA | 0.420 | 85.3 |
| E152C S375C-P1-L11-P1 | | |
| CD74 Milatuzumab DANAPA | 0.118 | 109.4 |
| E152C S375C-P1-L18-P2 | | |
| CD74 Milatuzumab DANAPA | 0.045 | 259.7 |
| E152C S375C-P1-L24-P1 | | |
| CD74 Milatuzumab DANAPA | 0.584 | 82.5 |
| E152C S375C-P1-L9-P1 | | |
| CD74 Milatuzumab DANAPA | 0.124 | 98.4 |
| E152C S375C-P1-L16-P2 | | |
| CD74 Milatuzumab DANAPA | 0.338 | 99.8 |
| E152C S375C-P1-L10-P1 | | |
| CD74 Milatuzumab DANAPA | 0.144 | 114.8 |
| E152C S375C-P1-L4-P1 | | |
| CD74 Milatuzumab DANAPA | 0.495 | 82.4 |
| E152C S375C-P1-L20-P1 | | |
| CD74 Milatuzumab DANAPA | 0.184 | 134.2 |
| E152C S375C-P1-L26-P1 | | |
| CD74 Milatuzumab DANAPA | 0.485 | 106.7 |
| E152C S375C-P1-L22-P1 | | |
| CD74 Milatuzumab DANAPA | 0.282 | 93.1 |
| E152C S375C-P1-L15-P2 | | |
| CD74 Milatuzumab DANAPA | 0.168 | 132.7 |
| E152C S375C-P1-L17-P3 | | |
| CD74 Milatuzumab DANAPA | 0.032 | 78.8 |
| E152C S375C-P1-L17-P4 | | |
| CD74 Milatuzumab DANAPA | 0.438 | 73.7 |
| E152C S375C-P1-L25-P1 | | |
| CD74 Milatuzumab DANAPA | 0.147 | 127.3 |
| E152C S375C-P1-L31-P2 | | |
| CD74 Milatuzumab DANAPA | 0.299 | 88.2 |
| E152C S375C-P1-L8-P1 | | |
| CD74 Milatuzumab DANAPA | 0.280 | 109.3 |
| E152C S375C-P1-L34-P1 | | |
| CD74 Milatuzumab DANAPA | 0.191 | 124.1 |
| E152C S375C-P1-L23-P1 | | |
| P1 | 0.338 | 88.6 |
| CD74 Milatuzumab DANAPA | 0.284 | 90.3 |
| E152C S375C-P1-L29-P2 | | |
| CD74 Milatuzumab DANAPA | 0.226 | 92.2 |
| E152C S375C-P2-L29-P1 | | |
| CD74 Milatuzumab DANAPA | 0.069 | 138.8 |
| E152C S375C-P1-L19-P2 | | |

### Example 6. In Vitro Assessment of Inhibition of Cell Proliferation and Survival with Dual ADCs and BCL-xL ADCs Cell Lines

The CD74 MCL-li and BCL-xLi antibody drug conjugates and the CD74 BCL-xL antibody drug conjugates were tested against two endogenous cancer cell lines.
Kasumi-6: ATCC No. CRL-2775 cultured in RPMI 1640 medium with 2 mM L-glutamine adjusted to contain 1.5 g/L sodium bicarbonate, 4.5 g/L glucose, 10 mMv HEPES, and 1.0 mM sodium pyruvate supplemented with 2 ng/ml human recombinant granulocyte macrophage colony stimulating factor (GM-CSF) and 20% FBS
EOL-1: DSMZ No. ACC-386 cultured in RPMI-1640+10% FBS

The CD48 MCL-li and BCL-xLi antibody drug conjugates and the CD48 BCL-xL antibody drug conjugates were tested against two endogenous cancer cell lines.
AMO-1: DSMZ No. ACC-538 cultured in RPMI-1640+20% FBS
KMS-27: JCRB No. JCRB1188 cultured in RPMI-1640+10% FBS

The EphA2 antibody drug conjugates were tested against one endogenous cancer cell line.
HCC-38: ATCC No. CRL-2314 cultured in RPMI-1640+10% FBS

The Her2 antibody drug conjugates were tested against three endogenous cancer cell lines.
HCC-38: ATCC No. CRL-2314 cultured in RPMI-1640+10% FBS
NCI-N87: ATCC No. CRL-5822 cultured in RPMI-1640+10% FBS
OCUM-1: JCRB No. JCRB0192 cultured in DMEM+0.5 mM sodium pyruvate+10% FBS

The PCAD antibody drug conjugates were tested against nine endogenous cancer cell lines.
BICR-22: ECACC No. 04072106 cultured in DMEM+2 mM Glutamine+2% FBS+0.4 micrograms/ml Hydrocortisone
HCC-38: ATCC No. CRL-2314 cultured in RPMI-1640+10% FBS
T3M-10: RCB No. RCB1020 cultured in Ham's F-12 Nutrient Mixture+10% FBS
   Inhibition of cell proliferation and survival

The ability of the MCL-1 and BCL-xL antibody drug conjugates and the BCL-xL antibody drug conjugates to inhibit cell proliferation and survival was assessed using the Promega CellTiter-Glo® proliferation assay.

Cell lines were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cell viability and cell density were determined using a cell counter (Vi-Cell XR Cell Viability Analyzer, Beckman Coulter). Cells with higher than 85% viability were seeded in white clear bottom 384-well TC treated plates (Corning cat. #3765). Cells were seeded at a density of 1,000 cells per well in 45 L μof standard growth media. Plates were incubated at 5% CO₂, 37° C. overnight in a tissue culture incubator. The next day, free MCL-1 payload (P1), free BCL-xL payload (P5 and P6) and targeting MCL-1 and BCL-xL ADCs and targeting BCL-xL ADCs were prepared at 10X in standard growth media. The prepared treatments were added to the cells resulting in final concentrations of 0.01-600 nM and a final volume of 50 μL per well. Each drug concentration was tested in quadruplets. Plates were incubated at 5% CO₂, 37° C. for 5 days in a tissue culture incubator, after which cell viability was assessed through the addition of 25 L μof CellTiter Glo® (Promega, cat #G7573), a reagent which lyses cells and measures total adenosine triphosphate (ATP) content. Plates were incubated at room temperature for 10 minutes to stabilize luminescent signals prior to reading using a luminescence reader (EnVision Multilabel Plate Reader, PerkinElmer). To evaluate the effect of the drug treatments, luminescent counts from wells containing untreated cells (100% viability) were used to normalize treated samples. A variable slope model was applied to fit a nonlinear regression curve to the data in GraphPad PRISM version 7.02 software. IC50 and Amax values were extrapolated from the resultant curves.

The concentrations of treatment required to inhibit 50% of cell growth or survival (IC50) were calculated with representative IC50 values of the cell lines tested summarized in Tables 3-7.

The representative CD48 and CD74 cancer cell lines were shown to be sensitive to the MCL-1 payload, P1, with IC50 values ranging from 0.685-36 nM activity. KMS-27, Kasumi-6 and EOL-1 were sensitive to the BCLxL payloads, P5 and P6. The CD48 targeting MCL-1 and BCL-xL ADC, NY920-P1-L19-P6, tested on AMO-1 demonstrated in vitro efficacy. The CD48 targeting BCL-xL ADCs, NY920-P5-L12-P5 and NY920-L11C-P25, tested on KMS-27 demonstrated in vitro efficacy. The CD74 targeting MCL-1 and BCL-xL ADC, CD74 IgG-P1-L19-P6, tested on Kasumi-6 and EOL-1 demonstrated in vitro efficacy. The CD74 targeting BCL-xL ADCs, VHmil x VKlaNQ-P5-L12-P5 and VHmil x VKlaNQ-L11C-P25, tested on Kasumi-6 and EOL-1 demonstrated in vitro efficacy.

The representative EphA2, Her2 and PCAD cancer cell lines were shown to be sensitive to the BCL-xL payload, P25, with IC50 values ranging from 10.9-67.8 nM. HCC-38 was sensitive to the EphA2 targeting BCL-xL ADC with an IC50 of less than 10 nM. The Her2 targeting BCL-xL ADCs were most potent across the three representative cancer cell lines with IC50s ranging from 0.047-1.01 nM. The PCAD targeting BCL-xL ADCs were broad in activity and potent across the three representative cancer cell lines with IC50s ranging from 0.056-95.3 nM.

These studies indicate that MCL-1 and BCL-xL ADCs and BCL-xL ADCs were capable of inhibiting cell proliferation on various hematological cancer cell lines expressing CD48 and CD74. In addition, these studies indicate that the BCL-xL ADCs were capable of inhibiting cell proliferation on various solid tumor cancer cell lines expressing EphA2, Her2 and PCAD.

TABLE 3
CD48 ADC Cytotoxicity

| | |
|---|---|
| AMO-1 | KMS-27 |

| | IC50 | Amax | IC50 | Amax |
|---|---|---|---|---|
| Compound | (nM) | (Span) | (nM) | (Span) |
| NY920-P1-L19-P6 | 0.700 | 70.5 | >10 | n/a |
| NY920-P5-L12-P5 | n/a | n/a | >10 | 68.6 |
| NY920-L11C-P25* | n/a | n/a | >10 | 18.2 |
| P5 | n/a | n/a | <5 | n/a |
| P6 | n/a | n/a | 7.89 | 93.7 |
| P1 | 3.80 | 95.8 | 5.23 | 96.7 |

*preparation of L11C-P25 is described in PCT Application PCT/US2021/060620, incorporated herein by reference in its entirety.

TABLE 4
CD74 ADC Cytotoxicity

| | |
|---|---|
| Kasumi-6 | EOL-1 |

| | IC50 | Amax | IC50 | Amax |
|---|---|---|---|---|
| Compound | (nM) | (Span) | (nM) | (Span) |
| VHmil × VK1aNQ- | 1.36 | 86.7 | 0.184 | 94.1 |
| P1-L19-P6 | | | | |
| VHmil × VK1aNQ - | 0.612 | 34.3 | <10 | 134.3 |
| P5-L12-P5 | | | | |
| VHmil × VK1aNQ - | <10 | 97.5 | <10 | n/a |
| L11C-P25* | | | | |
| P5 | 11.9 | 84.5 | 0.488 | 94.9 |
| P6 | <10 | n/a | <10 | n/a |
| P1 | 36.0 | 111.4 | 0.685 | 98.4 |

*preparation of L11C-P25 is described in PCT Application PCT/US2021/060620.

TABLE 5
EphA2 ADC Cytotoxicity

HCC-38

| Compound | IC50 (nM) | Amax (Span) |
|---|---|---|
| 1C1-P5-L12-P5 | <10 | 100.6 |
| P25* | 10.9 | 77.0 |

*preparation of P25 is described in PCT Application PCT/US2021/060620.

TABLE 6
Her2 ADC Cytotoxicity

| | OCUM-1 | | NCI-N87 | | HCC-38 | |
|---|---|---|---|---|---|---|
| | IC50 | Amax | IC50 | Amax | IC50 | Amax |
| Compound | (nM) | (Span) | (nM) | (Span) | (nM) | (Span) |
| Trastuzumab- | 0.178 | 84.3 | 1.01 | 54.0 | 0.047 | 76.5 |
| P5-L12-P5 | | | | | | |
| P25* | 67.8 | 95.5 | 24.0 | 52.0 | 10.9 | 77.0 |

*preparation of P25 is described in PCT Application PCT/US2021/060620

TABLE 7
PCAD ADC Cytotoxicity

| | HCC-38 | | T3M-10 | | BICR-22 | |
|---|---|---|---|---|---|---|
| | IC50 | Amax | IC50 | Amax | IC50 | Amax |
| Compound | (nM) | (Span) | (nM) | (Span) | (nM) | (Span) |
| NOV169N31Q- | 0.056 | 67.4 | 95.3 | 118.1 | >10 | 35.6 |
| P5-L12-P5 | | | | | | |
| P25* | 10.9 | 77.0 | 15.9 | 84.1 | 39.7 | 44.1 |

*preparation of P25 is described in PCT Application PCT/US2021/060620.

### Example 7. In Vivo Therapeutic Effect of Three CD48-Targeting Dual ADCs in H929 Multiple Myeloma Model

### Materials and methods

The ADCs below were prepared and characterized according to the conjugation procedure described in Example 3 above.

IgG1-CysmAb Fc silent_P1-L19-P2
anti-CD48 MEM_CysmAb Fc silent
anti-CD48 MEM_CysmAb Fc silent_P1-L19-P2
* Anti-CD-48 MEM_CysmAb Fc silent is SGN-48A with DANAPA Fc silencing mutation.

H929 cells, obtained from ATCC, were cultured in RPMI supplemented with 10% FBS. Cells were resuspended in 100% matrigel (BD Biosciences) and 0.1 ml containing 5×10⁶ cells were subcutaneously inoculated into the right flank of female SCID mice, provided by Charles River. When tumors reached the appropriate volume, mice were randomized, 8 animals per group, using Easy stat software. IgGI-CysmAb Fc silent_P1-L19-P2, anti-CD48 MEM_CysmAb Fc silent and anti-CD48 MEM_CysmAb Fc silent_P1-L19-P2, were injected at 30 mg/kg once IV in PBS. Mice body weight was monitored three times a week and tumor size measured using electronic calipers. Tumor volume was estimated by measuring the minimum and maximum tumor diameters using the formula: (minimum diameter)²(maximum diameter)/2. The last day with at least half of control animals still present in the study (d16), tumor growth inhibition was calculated using the formula:

$$( 1 - \frac{Median ( DTV at Dx in treated group )}{Median ( DTV at Dx in Control group )} ) \times 100$$

With DTV (Delta Tumor Volume) at Dx, calculated being TV at Dx - TV at Randomization.

Mice were sacrificed at the first measurement for which tumor volume exceeded 2000 mm³ or at the first signs of animal health deterioration. All experiments were conducted in accordance with the French regulations in force in 2018 after approval by Servier Research Institute (IdRS) Ethical Committee. SCID mice were maintained according to institutional guidelines.

### Results

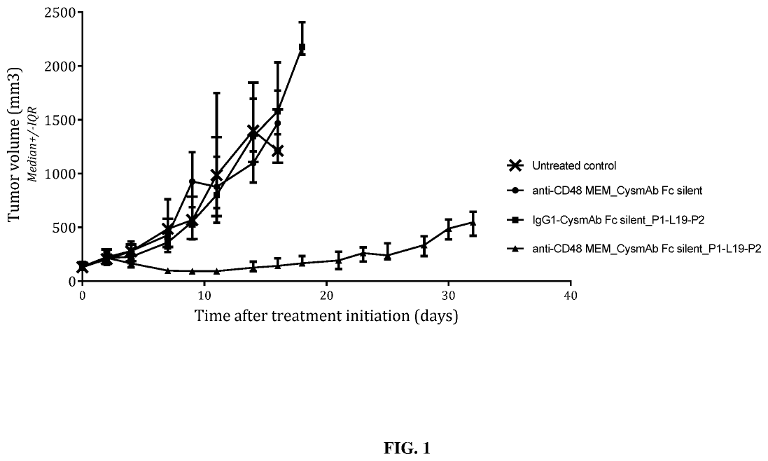

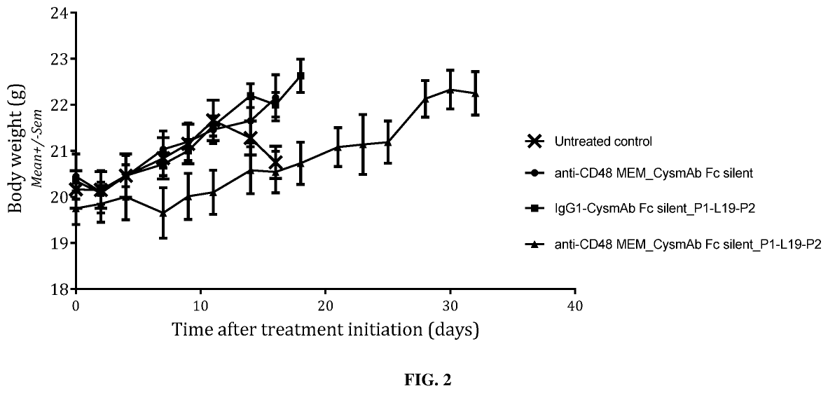

The efficacy of anti-CD48 dual ADCs on H929 xenografts is illustrated in FIG. 1. Treatment was started 10 days post tumor cells inoculation (median size: 135 mm³). anti-CD48 MEM_CysmAb Fc silent (CD48 naked antibody FS), IgGI-CysmAb Fc silent_P1-L19-P2 (non-targeting ADC FS), anti-CD48 MEM_CysmAb Fc silent_P1-L19-P2 were administered once IV at 30 mg/kg. After 16 days after the treatment, a very limited Tumor Growth Inhibition (% TGI) was observed upon treatment with the non-targeting ADC FS (2 payloads) (TGI=−30.07%), or with the CD48 naked antibody FS (TGI=−22.91%) as depicted in FIG. 1 and Table 8. A higher inhibition was induced by the anti-CD48 MEM_CysmAb Fc silent_P1-L19-P2 at 30 mg/kg (TGI=98.23%, p<0.001 compared to control group). No clinically relevant body weight loss (BWL) or other clinical signs due to the treatment were observed (FIG. 2).

TABLE 8
H929 tumor growth inhibition upon treatment with
IgG1-CysmAb Fc silent_P1-L19-P2, anti-CD48 MEM_CysmAb
Fc silent, anti-CD48 MEM_CysmAb Fc silent_P1-
L19-P2 at 30 mg/kg, administered once IV (n = 8).

| | Dose | | % BWL |
|---|---|---|---|
| | (mg/kg)/ | % TGI | (at the |
| Treatment | Schedule | (d 16) | nadir) |
| IgG1-CysmAb Fc silent_P1-L19-P2 | 30 QD, IV | −30.07 | −1.34 (D 2) |
| anti-CD48 MEM_CysmAb Fc silent | 30 QD, IV | −22.91 | −0.26 (D 7) |
| anti-CD48 MEM_CysmAb Fc | 30 QD, IV | 98.23 | −3.55 (D 7) |
| silent_P1-L19-P2 | | | |

* Anti-CD-48 MEM_CysmAb Fc silent is SGN-48A with DANAPA Fc silencing mutation.

### Example 8. In Vivo Therapeutic Effect of Six CD48-Targeting Dual ADCs in H929 Multiple Myeloma Model

### Materials and methods

The ADCs below were prepared and characterized according to the conjugation procedure described in Example 3 above.

IgG1-CysmAb Fc WT_P1-L19-P2
anti-CD48 MEM_CysmAb Fc WT_P1-L19-P2
anti-CD48 MEM_CysmAb Fc WT_P1-L29-P2
anti-CD48 MEM_CysmAb Fc WT_P2-L29-P1
anti-CD48 MEM_CysmAb Fc WT_P1-L31-P2
anti-CD48 MEM_CysmAb Fc WT_P1-L32-P2
anti-CD48 MEM_CysmAb Fc WT_P1-L30-P2
* Anti-CD-48 MEM_CysmAb Fc WT is SGN-48A.

H929 cells, obtained from ATCC, were cultured in RPMI supplemented with 10% FBS. Cells were resuspended in 100% matrigel (BD Biosciences) and 0.1 ml containing 5×10⁶ cells were subcutaneously inoculated into the right flank of female SCID mice, provided by Charles River. When tumors reached the appropriate volume, mice were randomized, 6 animals per group, using Easy stat software. IgG1-CysmAb Fc WT_P1-L19-P2, anti-CD48 MEM_CysmAb Fc WT_P1-L19-P2, anti-CD48 MEM_CysmAb Fc WT_P1-L29-P2, anti-CD48 MEM_CysmAb Fc WT_P2-L29-P1, anti-CD48 MEM_CysmAb Fc WT_P1-L31-P2, anti-CD48 MEM_CysmAb Fc WT_P1-L32-P2, anti-CD48 MEM_CysmAb Fc WT_P1-L30-P2 were injected at 30 mg/kg once IV in PBS. Mice body weight was monitored three times a week and tumor size measured using electronic calipers. Tumor volume was estimated by measuring the minimum and maximum tumor diameters using the formula: (minimum diameter)²(maximum diameter)/2. The last day with at least half of control animals still present in the study (d11), tumor growth inhibition was calculated using the formula:

$$( 1 - \frac{Median ( DTV at Dx in treated group )}{Median ( DTV at Dx in Control group )} ) \times 100$$

With DTV (Delta Tumor Volume) at Dx, calculated being TV at Dx - TV at Randomization.

Mice were sacrificed at the first measurement for which tumor volume exceeded 2000 mm³ or at the first signs of animal health deterioration. All experiments were conducted in accordance with the French regulations in force in 2018 after approval by Servier Research Institute (IdRS) Ethical Committee. SCID mice were maintained according to institutional guidelines. Results

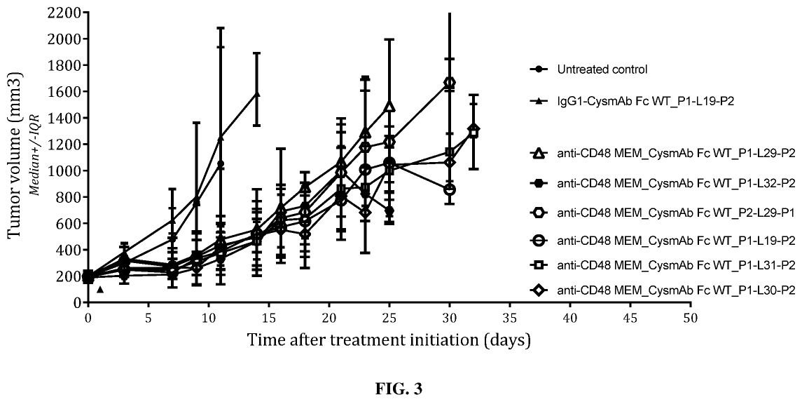

The efficacy of different dual anti-CD48 ADCs on H929 xenografts is illustrated in FIG. 3. Treatment was started 11 days post tumor cells inoculation (median size: 197 mm³). IgGI-CysmAb Fc WT_P1-L19-P2 (non targeting ADC), anti-CD48 MEM_CysmAb Fc WT_P1-L19-P2, anti-CD48 MEM_CysmAb Fc WT_P1-L29-P2, anti-CD48 MEM_CysmAb Fc WT_P2-L29-P1, anti-CD48 MEM_CysmAb Fc WT_P1-L31-P2, anti-CD48 MEM_CysmAb Fc WT_P1-L32-P2, anti-CD48 MEM_CysmAb Fc WT_P1-L30-P2 were administered once IV at 30 mg/kg.

On day 11, a very limited Tumor Growth Inhibition (% TGI) was observed upon treatment with the non-targeting ADC (TGI=−21.93%), as depicted in FIG. 3 and Table 9. A higher inhibition was induced by the six dual CD48-targeting ADC with TGI ranging from 71.59% to 80.04% as described in Table 9 (p<0.01 as compared to control group).

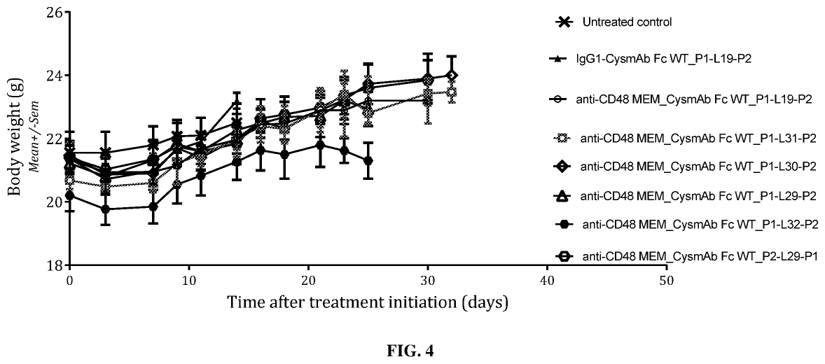

No clinically relevant body weight loss (BWL) or other clinical signs due to the treatment were observed (FIG. 4).

TABLE 9
H929 tumor growth inhibition upon treatment with IgG1-CysmAb
Fc WT_P1-L19-P2, anti-CD48 MEM_CysmAb Fc WT_P1-L19-P2,
anti-CD48 MEM_CysmAb Fc WT_P1-L29-P2, anti-CD48
MEM_CysmAb Fc WT_P2-L29-P1, anti-CD48
MEM_CysmAb Fc WT_P1-L31-P2, anti-CD48
MEM_CysmAb Fc WT_P1-L32-P2 and anti-
CD48 MEM_CysmAb Fc WT_P1-L30-P2
administrated at 30 mg/kg once
IV, (n = 6).

| | Dose | | % BWL |
|---|---|---|---|
| | (mg/kg)/ | % TGI | (at the |
| Treatment | Schedule | (d 11) | nadir) |
| IgG1-CysmAb Fc | 30 QD, IV | −21.93 | −3.02 (D 3) |
| WT_P1-L19-P2 | | | |
| anti-CD48 MEM_CysmAb | 30 QD, IV | 77.10 | −1.98 (D 3) |
| Fc WT_P1-L19-P2 | | | |
| anti-CD48 MEM_CysmAb | 30 QD, IV | 72.47 | −1.95 (D 3) |
| Fc WT_P1-L29-P2 | | | |
| anti-CD48 MEM_CysmAb | 30 QD, IV | 76.78 | −1.72 (D 3) |
| Fc WT_P2-L29-P1 | | | |
| anti-CD48 MEM_CysmAb | 30 QD, IV | 79.21 | −0.93 (D 3) |
| Fc WT_P1-L31-P2 | | | |
| anti-CD48 MEM_CysmAb | 30 QD, IV | 80.04 | −2.12 (D 3) |
| Fc WT_P1-L32-P2 | | | |
| anti-CD48 MEM_CysmAb | 30 QD, IV | 71.59 | −2.8 (D 3) |
| Fc WT_P1-L30-P2 | | | |

### Example 9. In Vivo Therapeutic Effect of Three CD48-Targeting Dual ADCs in KMS-21-BM Multiple Myeloma Model

### Materials and methods

The ADCs below were prepared and characterized according to the conjugation procedure described in Example 3 above.

IgG1-CysmAb Fc silent_P1-L29-P2
anti-CD48 MEM_CysmAb Fc silent*
anti-CD48 MEM_CysmAb Fc silent_P1-L29-P2
*Anti-CD-48 MEM_CysmAb Fc silent is SGN-48A with DANAPA Fc silencing mutation.

KMS-21-BM cells, obtained from JCRB, were cultured in RPMI supplemented with 10% FBS. Cells were resuspended in 100% matrigel (BD Biosciences) and 0.1 ml containing 13×10⁶ cells were subcutaneously inoculated into the right flank of female NSG mice, provided by Jackson Laboratory. When tumors reached the appropriate volume, mice were randomized, 6 animals per group, using Easy stat software. IgGI-CysmAb Fc silent_P1-L29-P2, anti-CD48 MEM_CysmAb Fc silent, anti-CD48 MEM CysmAb Fc silent_P1-L29-P2 at 30 mg/kg were injected once IV in PBS. Mice body weight was monitored three times a week and tumor size measured using electronic calipers. Tumor volume was estimated by measuring the minimum and maximum tumor diameters using the formula: (minimum diameter)²(maximum diameter)/2. The last day with all control animals in the study (D14), tumor growth inhibition was calculated using the formula:

$$( 1 - \frac{Median ( DTV at Dx in treated group )}{Median ( DTV at Dx in Control group )} ) \times 100$$

With DTV (Delta Tumor Volume) at Dx, calculated being TV at Dx - TV at Randomization. Mice were sacrificed at the first measurement for which tumor volume exceeded 2000 mm³ or at the first signs of animal health deterioration. All experiments were conducted in accordance with the French regulations in force in 2018 after approval by Servier Research Institute (IdRS) Ethical Committee. NSG mice were maintained according to institutional guidelines.

### Results

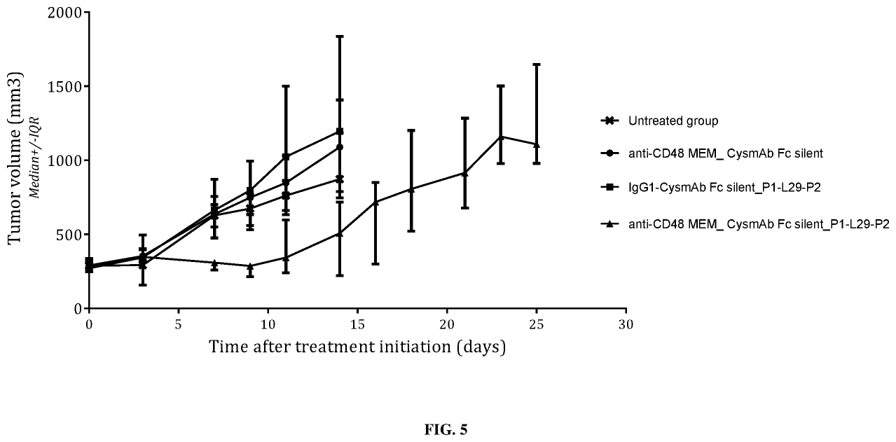

The efficacy of the dual anti-CD48 ADC on KMS-21-BM xenografts is illustrated in FIG. 5. Treatment was started 18 days post tumor cells inoculation (median size: 291 mm³). IgG1-CysmAb Fc silent_P1-L29-P2 (non-targeting silent ADC), anti-CD48 MEM_CysmAb Fc silent (CD48-targeting silent naked mAb), anti-CD48 MEM_CysmAb Fc silent_P1-L29-P2 were administered once IV at 30 mg/kg.

After 14 days after the treatment, a very limited Tumor Growth Inhibition (% TGI) was observed upon treatment with the non-targeting silent ADC (2 payloads) (TGI=−51.6%), or with the CD48-targeting silent naked antibody (TGI=−28.01%) as depicted in FIG. 5 and Table 10. A higher inhibition was induced by the anti-CD48 MEM_CysmAb Fc silent_P1-L29-P2 at 30 mg/kg (TGI=73.24%, p<0.001 compared to control group).

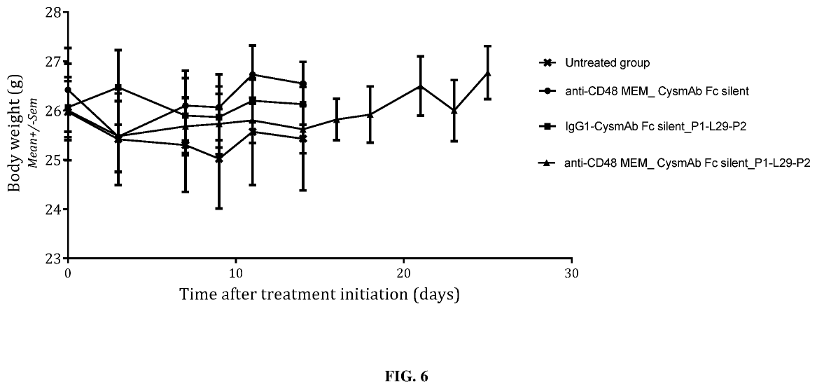

No clinically relevant body weight loss (BWL) or other clinical signs due to the treatment were observed (FIG. 6).

TABLE 10
KMS-21-BM tumor growth inhibition upon treatment
with IgG1-CysmAb Fc silent_P1-L29-P2, anti-CD48
MEM_CysmAb Fc silent, anti-CD48 MEM_CysmAb Fc
silent_P1-L29-P2 administered once IV (n = 6).

| | Dose (mg/kg)/ | % TGI | % BWL (at | |
|---|---|---|---|---|
| Treatment | Schedule | (d14) | the nadir) | |
| IgG1-CysmAb Fc | 30 QD, IV | −51.6 | −1.57 | (D18) |
| silent_P1-L29-P2 | | | | |
| anti-CD48 MEM_CysmAb | 30 QD, IV | −28.01 | −3.5 | (D3) |
| Fc silent* | | | | |
| anti-CD48 MEM_CysmAb | 30 QD, IV | 73.24 | −2 | (D3) |
| Fc silent_P1-L29-P2 | | | | |

*Anti-CD-48 MEM_CysmAb Fc silent is SGN-48A with DANAPA Fc silencing mutation.

### Example 10. In Vivo Therapeutic Effect of Three CD48-Targeting Dual ADCs in KMS27 Multiple Myeloma Model

### Materials and methods

The ADCs below were prepared and characterized according to the conjugation procedure described in Example 3 above.

IgG1-CysmAb Fc silent_P1-L19-P2
anti-CD48 MEM102_CysmAb Fc silent*
anti-CD48 MEM102_CysmAb Fc silent_P1-L19-P2
*Anti-CD-48 MEM102_CysmAb Fc silent is SGN-48A with DANAPA Fc silencing mutation.

KMS27 cells, obtained from JCRB, were cultured in RPMI supplemented with 10% FBS. Cells were resuspended in 50% matrigel (BD Biosciences) and 0.1 ml containing 11×10⁶ cells were subcutaneously inoculated into the right flank of female NSG mice, provided by Jackson Laboratory. When tumors reached the appropriate volume, mice were randomized, 6 animals per group, using Easy stat software. IgGI-CysmAb Fc silent_P1-L19-P2, anti-CD48 MEM102_CysmAb Fc silent and anti-CD48 MEM102_CysmAb Fc silent_P1-L19-P2 (10 and/or 30 mg/kg) were injected once IV in PBS. Mice body weight was monitored three times a week and tumor size measured using electronic calipers. Tumor volume was estimated by measuring the minimum and maximum tumor diameters using the formula: (minimum diameter)²(maximum diameter)/2. Tumor growth inhibition was calculated on day 9, when all animals from control group were sacrificed using the formula:

$$( 1 - \frac{Median ( DTV at Dx in treated group )}{Median ( DTV at Dx in Control group )} ) \times 100$$

With DTV (Delta Tumor Volume) at Dx, calculated being TV at Dx - TV at Randomization.

Mice were sacrificed at the first measurement for which tumor volume exceeded 2000 mm³ or at the first signs of animal health deterioration. All experiments were conducted in accordance with the French regulations in force in 2018 after approval by Servier Research Institute (IdRS) Ethical Committee. NSG mice were maintained according to institutional guidelines.

### Results

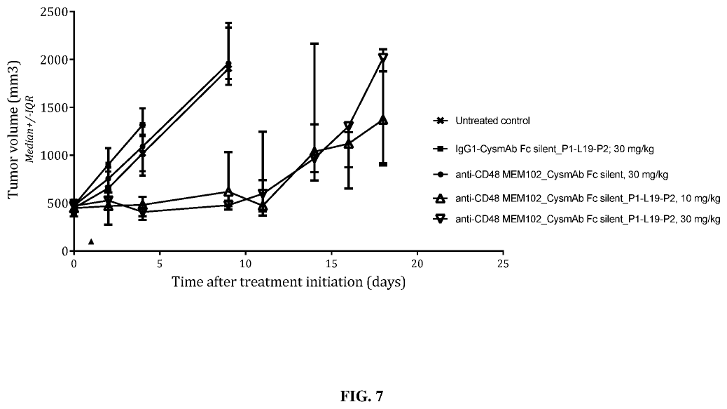

The efficacy of the dual anti-CD48 ADC on KMS27 xenografts is illustrated in FIG. 7. Treatment was started 18 days post tumor cells inoculation (median size: 449 mm³). IgGI-CysmAb Fc silent_P1-L19-P2 (non targeting ADC FS), anti-CD48 MEM102_CysmAb Fc silent (CD48-targeting naked mAb FS) and anti-CD48 MEM102_CysmAb Fc silent_P1-L19-P2 were administered once IV at 10 and/or 30 mg/kg.

On day 9, no activity was observed upon treatment with the non-targeting ADC FS (2 payloads) nor with the CD48 naked antibody FS administrated at 30 mg/kg (TGI=−3.22%) as depicted in FIG. 7 and Table 11. At the opposite, antitumor activity was observed under the anti-CD48 MEM_CysmAb Fc silent_P1-L19-P2 at 10 and 30 mg/kg without dose response (TGI=84.38% and 98.02% at 10 mg/kg and 30 mg/kg respectively, p<0.01 compared to control group).

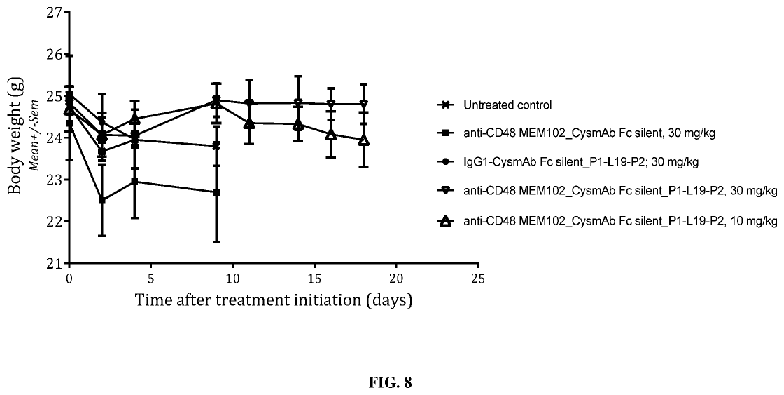

No clinically relevant body weight loss (BWL) or other clinical signs due to the treatment with anti-CD48 MEM102_CysmAb Fc silent_P1-L19-P2 were observed (FIG. 8).

TABLE 11
KMS27 tumor growth inhibition upon treatment with IgG1-
CysmAb Fc silent_P1-L19-P2, anti-CD48 MEM102_CysmAb
Fc silent and anti-CD48 MEM102_CysmAb Fc silent_{—}
P1-L19-P2 at 10 and/or 30 mg/kg, once IV (n = 6).

| | Dose (mg/kg)/ | % TGI | % BWL (at | |
|---|---|---|---|---|
| Treatment | Schedule | (d9) | the nadir) | |
| IgG1-CysmAb Fc | 30 QD, IV | NA | −4.17 | (D4) |
| silent_P1-L19-P2 | | | | |
| anti-CD48 MEM102_CysmAb | 30 QD, IV | −3.22 | −7.62 | (D2) |
| Fc silent | | | | |
| anti-CD48 MEM102_CysmAb | 30 QD, IV | 98.02 | −3.15 | (D4) |
| Fc silent_P1-L19-P2 | | | | |
| anti-CD48 MEM102_CysmAb | 10 QD, IV | 84.38 | −3.53 | (D21) |
| Fc silent_P1-L19-P2 | | | | |

*Anti-CD-48 MEM_CysmAb Fc silent is SGN-48A with DANAPA Fc silencing mutation.

### Example 11. In Vivo Efficacy Studies in KMS-27 S.C. MM Tumor Model

### Materials and Methods

Human multiple myeloma KMS-27 cells were cultured at 37° C. (atmosphere of 5% CO₂) in RPMI 1640 medium (BioConcept Amimed, #1-41F01-I) supplemented with 10% FBS (BioConcept Amimed, #2-01F10), 2 mM L-glutamine (BioConcept Amimed, #5-10K50-H), 1 mM sodium pyruvate (BioConcept Amimed, #5-60F00-H) and 10 mM HEPES (BioConcept Amimed, #5-31F00-H). To establish KMS-27 xenografts, cells were harvested and re-suspended in a 1:1 v/v mixture of HBSS (Gibco, #14175) and Matrigel (Corning #354234). A total of 7.5×10⁶ KMS-27 cells were injected subcutaneously in the right flanks of female NSG mice (Charles River, Germany) in a volume of 100 μL. Tumor growth and change in body weight was monitored regularly post cell inoculation and animals were randomized into treatment groups (n=6) with a mean tumor volume of about 150 mm³.

For KMS-27 efficacy, CD48-P1-L12-P4 and CD48-P1-L17-P4 ADCs were both dosed as single agents. ADCs were administered intravenously (IV) once at the start of treatment at 5 mg/kg. All ADCs were dosed at 10 ml/kg based on the individual mouse body weight and formulated accordingly in sterile PBS on the day of treatment.

As a measure of efficacy, tumor growth delay (TGD) was calculated according to the following formula: time for tumor to regrow until initial volume in 3/6 mice (or 50% of the mice). Complete response (CR) was defined by tumor regression >90%.

Tolerability (% change in body weight from TO (start of treatment) on day 14) was calculated according to the following formula: (body weight at day 14 - body weight at start of treatment/body weight at start of treatment)*100. Values are presented in the data table as mean. The efficacy and tolerability results are reported in Table 12.

TABLE 12
Efficacy studies in KMS-27 s.c. MM tumor
model (Cs66-21) with CD48 WT ADCs

| | Efficacy | | Tolerability | |
|---|---|---|---|---|
| | | TGD | % BW | |
| 5 mg/kg IV | CR | (days) | (d14) | other |

| | | | |
|---|---|---|---|
| CD48-P1-L12-P4 | 6/6 | 53 | 2 |
| CD48-P1-L17-P4 | 6/6 | 31 | 0 |

TGD: time for tumor to regrow until initial volume in 3/6 mice (or 50% of the mice)
CR: complete response = tumor regression > 90%
*CD48 antibody is NY920 Cysmab WT.

### Example 12. In Vivo Efficacy Study in EOL-1 Luc Model

### Materials and Methods

Human leukemia EOL-1 luc cells were cultured at 37° C. (atmosphere of 5% CO₂) in RPMI 1640 medium supplemented with 10% FBS. To establish EOL-1 luc xenografts, cells were harvested and re-suspended in PBS. A total of 5×10⁶ EOL-1 luc cells were injected subcutaneously in the right flanks of female SCID/beige mice (Vital River Laboratories Research Models and Service, China) in a volume of 100 μL. Tumor growth and change in body weight was monitored regularly post cell inoculation and animals were randomized into treatment groups (n=6) with a mean tumor volume of about 130 nmm³.

For EOL-1 luc efficacy study #1, CD74-P1-L25-P1, CD74-P1-L21-P1 and CD74-P1-L20-P1 ADCs were all dosed in combination with ABT-199. For EOL-1 luc efficacy study #2, CD74-P1-L7-P1, CD74-P1-L6-P1, CD74-P1-L35-P1, CD74-P1-L23-P1 and CD74-P1-L36-P1 ADCs were all dosed in combination with ABT-199. For EOL-1 luc efficacy study #3, CD74-P1-L5-P1, CD74-P1-L6-P1, CD74-P1-L7-P1, CD74-P1-L33-P1, CD74-P1-L4-P1 and CD74-P1-L11-PI ADCs were all dosed in combination with ABT-199. For EOL-1 luc efficacy study #4, CD74-P1-L18-P2, CD74-P1-L16-P2, CD74-P1-L17-P2 and CD74-P1-L12-P2 ADCs were all dosed as single agents. For EOL-1 luc efficacy study #5, CD74-P1-L12-P2, CD74-P1-L12-P4 and CD74-P1-L17-P4 ADCs were all dosed as single agents. ADCs were administered intravenously (IV) once at the start of treatment at dose levels specified in Tables 13-17 immediately after dosing of ABT-199. ABT-199 was administered orally (PO) at 50 mg/kg once per day for 14 days. All test articles were dosed at 10 ml/kg based on the individual mouse body weight. The ADCs were formulated accordingly in sterile PBS on the day of treatment. ABT-199 was formulated by reconstituting in 10% Ethanol (absolute)+30% PEG300+59.5% Phosal 50PG+0.5% ION NaOH at a concentration of 5 mg/ml. Final formulation is stable for 7 days, protected from light at room temperature.

As a measure of efficacy, tumor growth delay (TGD) was calculated according to the following formula: time for tumor to regrow until initial volume in 3/6 mice (or 50% of the mice). Complete response (CR) was defined by tumor regression >90%.

Tolerability (% change in body weight from TO (start of treatment) on day 14) was calculated according to the following formula: (body weight at day 14 - body weight at start of treatment/body weight at start of treatment)*100. Values are presented in the data table as mean.

The efficacy and tolerability results are summarized in Tables 13-17

TABLE 13
Efficacy studies in EOL1-luc (leukemia xenograft
model, study #1) with CD74 DANAPA ADCs

| | |
|---|---|
| Efficacy | Tolerability |

| | | | | |
|---|---|---|---|---|
| | | TGD | % BW | |
| 2 mg/kg IV + ABT-199 | CR | (days) | (d14) | other |

| | | | |
|---|---|---|---|
| CD74-P1-L25-P1 | 5/6 | 21 → 42 | 2 |
| CD74-P1-L21-P1 | 5/6 | 21 → 42 | 4 |
| CD74-P1-L20-P1 | 4/6 | 21 → 42 | −1 |

ABT-199: 50 mg/kg p.o. qd for 14 days
TGD: time for tumor to regrow until initial volume in 3/6 mice
CR: complete response = tumor regression > 90%
→: selected for prolonged observation

TABLE 14
Efficacy studies in EOL1-luc (leukemia xenograft
model, study #2) with CD74 DANAPA ADCs

| | |
|---|---|
| Efficacy | Tolerability |

| | | | | |
|---|---|---|---|---|
| | | TGD | % BW | |
| 2 mg/kg IV + ABT-199 | CR | (days) | (d14) | other |

| | | | |
|---|---|---|---|
| CD74-P1-L7-P1 | 6/6 | 21 → 42 | −3 |
| CD74-P1-L6-P1 | 6/6 | 21 → 42 | −6 |
| CD74-P1-L35-P1 | 5/6 | 21 → 42 | −3 |
| CD74-P1-L23-P1 | 5/6 | 21 → 42 | −5 |
| CD74-P1-L36-P1 | 4/6 | 21 → 23 | −3 |

ABT-199: 50 mg/kg p.o. qd for 14 days
TGD: time for tumor to regrow until initial volume in 3/6 mice
CR: complete response = tumor regression > 90%
→: selected for prolonged observation

TABLE 15
Efficacy studies in EOL1-luc (leukemia xenograft
model, study #3) with CD74 DANAPA ADCs

| | |
|---|---|
| Efficacy | Tolerability |

| | | | | |
|---|---|---|---|---|
| | | TGD | % BW | |
| 2 mg/kg IV + ABT-199 | CR | (days) | (d14) | other |

| | | | |
|---|---|---|---|
| CD74-P1-L5-P1 | 6/6 | 21 → 42 | 2 |
| CD74-P1-L6-P1 | 6/6 | 21 → 42 | 4 |
| CD74-P1-L7-P1 | 6/6 | 21 → 42 | 5 |
| CD74-P1-L33-P1 | 5/6 | 21 → 42 | 2 |
| CD74-P1-L4-P1 | 5/6 | 21 | 3 |
| CD74-P1-L11-P1 | 4/6 | 21 | 6 |

ABT-199: 50 mg/kg p.o. qd for 14 days
TGD: time for tumor to regrow until initial volume in 3/6 mice
CR: complete response = tumor regression > 90%
→: selected for prolonged observation

TABLE 16
Efficacy studies in EOL1-luc (leukemia xenograft model, study #4)

| | Efficacy | | Tolerability | |
|---|---|---|---|---|
| | | TGD | % BW | |
| 12 mg/kg IV | CR | (days) | (d14) | other |

| | | | |
|---|---|---|---|
| CD74-P1-L18-P2 | 4/6 | >23 | 5 |
| CD74-P1-L16-P2 | 5/6 | 20 | 3 |
| CD74-P1-L17-P2) | 5/6 | >23 | 4 |
| CD74-P1-L12-P2 | 5/6 | >23 | 6 |

ABT-199: 50 mg/kg p.o. qd for 14 days
TGD: time for tumor to regrow until initial volume in 3/6 mice
CR: complete response = tumor regression > 90%

TABLE 17
Efficacy studies in EOL1-luc (leukemia xenograft model, study #5)

| | Efficacy | | Tolerability | |
|---|---|---|---|---|
| | | TGD | % BW | |
| 12 mg/kg IV | CR | (days) | (d14) | other |

| | | | |
|---|---|---|---|
| CD74-P1-L12-P2 | 3/6 | 17 | 2 |
| CD74-P1-L12-P4 | 4/6 | 14 | 3 |
| CD74-P1-L17-P4 | 3/6 | 17 | −3 |

ABT-199: 50 mg/kg p.o. qd for 14 days
TGD: time for tumor to regrow until initial volume in 3/6 mice (or 50% of the mice)
CR: complete response = tumor regression > 90%

### Example 13. In Vitro Assessment BCLxL Topo Dual ADCs and BCLxL BCL2 Dual ADCs

### Cell Lines

The BCLxLi/TOPOi dual antibody drug conjugates were tested against four endogenous lung cancer cell lines.
NCI-H441: ATCC No. HTB-174 cultured in RPMI-1640+10% FBS
The BCLxLi/TOPOi dual antibody drug conjugates were tested against four endogenous breast cancer cell lines.
HCC1419: ATCC No. CRL-2326 cultured in RPMI-1640+10% FBS
ZR-75-30: ATCC No. CRL-1504 cultured in RPMI-1640+10% FBS
UACC-812: ATCC No. CRL-1897 cultured in RPMI-1640+20% FBS+20 ng/mL EGF
The BCLxLi/BCL2i dual antibody drug conjugates were tested against one endogenous AML cancer cell line and one endogenous MM cancer cell line.
EOL-1: DSMZ No. ACC 386 cultured in RPMI-1640+10% FBS
KMS-27: JCRB No. JCRB1188 cultured in RPMI-1640+10% FBS
   Inhibition of cell proliferation and survival

The ability of the BCLxLi/TOPOi dual antibody drug conjugates and the BCLxLi/BCL2i dual antibody drug conjugates to inhibit cell proliferation and survival was assessed using the Promega CellTiter-Glo® proliferation assay. Monoloaded ADCs were included for comparison. The mono loaded ADCs tested were all DAR4 except Enhertu which was DAR8.

Cell lines were cultured in media that is optimal for their growth at 5% C02, 37° C. in a tissue culture incubator. Prior to seeding for the proliferation assay, the cells were split at least 2 days before the assay to ensure optimal growth density. On the day of seeding, adherent cells were lifted off tissue culture flasks using 0.05% trypsin. Cell viability and cell density were determined using a cell counter (Vi-Cell BLU Cell Viability Analyzer, Beckman Coulter). Cells with higher than 85% viability were seeded in white clear bottom 384-well TC treated plates (Corning cat. #3765). Cells were seeded at a density of 1,000 cells per well in 45 L μof standard growth media, with the exception of ZR-75-30 and UACC-812, which were seeded at a density of 2,000 cells per well in 45 L μof standard growth media. Plates were incubated at 5% C02, 37° C. overnight in a tissue culture incubator.

On the day of dosing all ADCs were prepared at 10× in standard growth media. The prepared BCLxLi/TOPOi and BCLxLi/BCL2i targeting ADC treatments were added to the cells resulting in final concentrations of 0.001-500 nM and a final volume of 50 μL per well. Each drug concentration was tested in quadruplets. The same day, cell viability of untreated cells was assessed through the addition of L of CellTiter Glo® (Promega, cat #G7573), a reagent which lyses cells and measures total adenosine triphosphate (ATP) content. Plates were incubated at room temperature for 10 minutes to stabilize the luminescent signals prior to reading. The luminescent signal for all plates was measured using a luminescence reader (PHERAstar FSX Plate Reader, BMG Labtech). This signal represents the cell viability of untreated cells on the day of dosing, or day 0.

Plates were incubated at 5% CO2, 37° C. for 5 days in a tissue culture incubator, after which cell viability was assessed through the addition of 25 L μof CellTiter Glo® (Promega, cat #G7573). Plates were incubated at room temperature for 10 minutes to stabilize luminescent signals. All assay plates were read using a luminescence reader (PHERAstar FSX Plate Reader, BMG Labtech). To evaluate the effect of the drug treatments, luminescent counts from wells containing untreated cells (100% viability) at day 5 and day 0 were used to normalize treated samples. A variable slope model was applied to fit a nonlinear regression curve to the data in GraphPad PRISM version 9.4.1 software. IC50 and Amax values were extrapolated from the resultant curves.

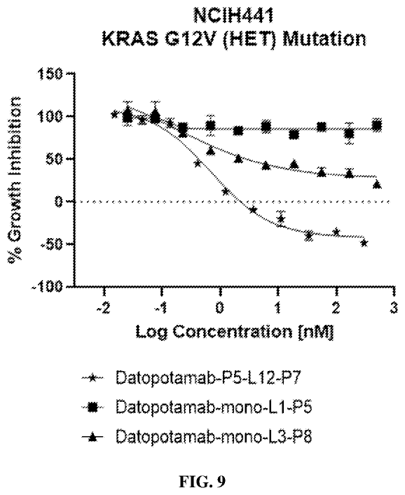

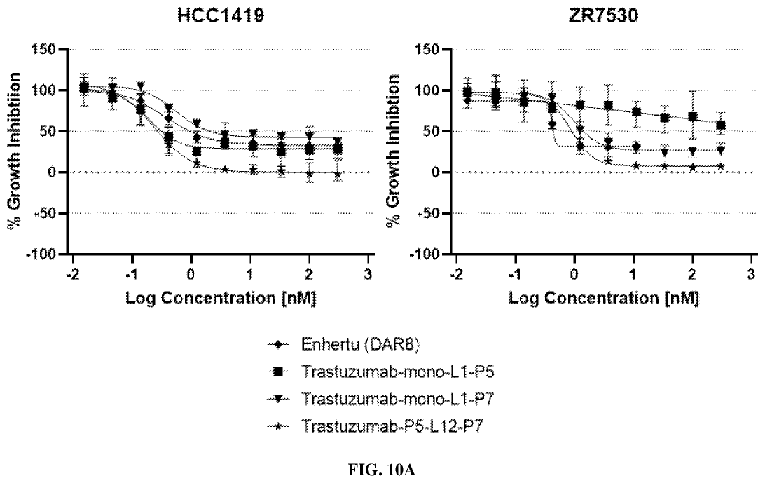

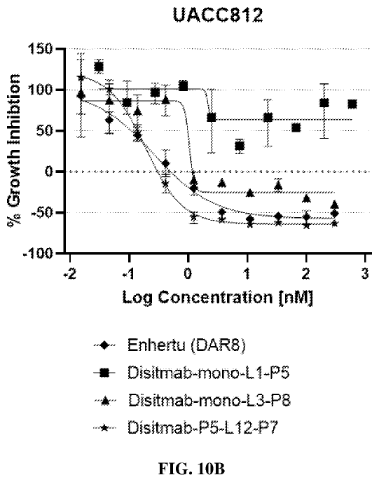

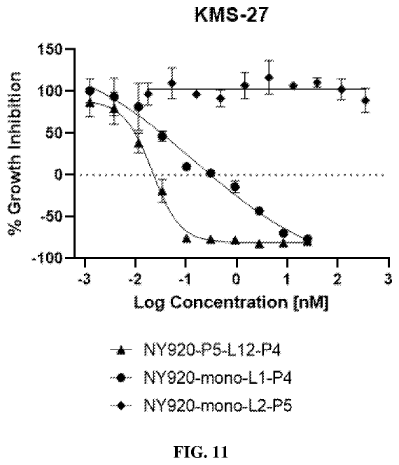

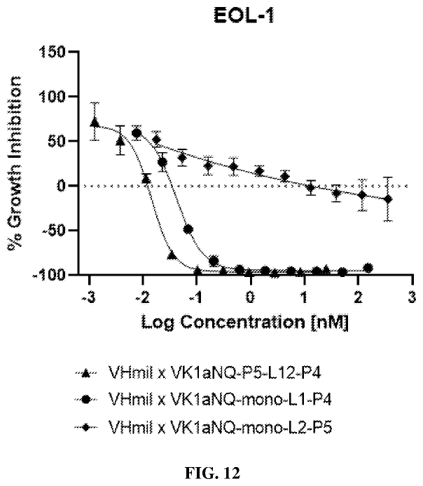

The dose response curves of representative cancer cell lines are shown in FIGS. 9-12. The concentrations of treatment required to inhibit 50% of cell growth or survival (IC50) were calculated with representative IC50 values of the cell lines tested summarized in Tables 18-21.

The representative lung cancer cell lines were shown to be sensitive to the ADCs with an IC50s of 0.692 - >5 nM activity. The TROP2 targeting BCLxLi/TOPOi ADCs demonstrated improved vitro efficacy relative to a BCLxLi mono ADC and a TOPOi mono ADC on the cell line tested. These studies indicate that a BCLxLi/TOPOi ADC is capable of inhibiting cell proliferation on a lung cancer cell line expressing TROP2.

The representative breast cancer cell lines were shown to be sensitive to the ADCs with IC50 values ranging from 0.191 - >50 nM. In general, the BCLxLi/TOPOi ADCs demonstrated improved cell growth inhibition compared to Enhertu. Both Trastuzumab and Disitimab demonstrated in vitro efficacy in cell growth inhibition as ADCs targeting HER2. These studies indicate that BCLxLi/TOPOi ADCs are capable of inhibiting cell proliferation on various breast cancer cell lines expressing HER2.

The representative AML and MM cell lines were shown to be sensitive to the ADCs with IC50 values ranging from 0.014 - >50 nM. The BCLxLi/BCL2i dual ADCs demonstrated improved cell growth inhibition compared to a BCLxLi mono ADC and a BCL2i mono ADC. These studies indicate that a BCLxLi/BCL2i ADC is capable of inhibiting cell proliferation on a MM cancer cell line expressing CD48 and an AML cancer cell line expressing CD74.

TABLE 18
BCLxLi/TOPOi dual ADC Cytotoxicity on a Lung Model
IC50 (nM) of Compounds in a Representative Lung Model

| Compound | NCI-H441 |
|---|---|
| Datopotamab-P5-L12-P7 | 0.692 |
| Datopotamab-mono-L1-P5 | — |
| Datopotamab-mono-L3-P8 | <5 |

TABLE 19
BCLxLi/TOPOi dual ADC Cytotoxicity on Breast Models
IC50 (nM) of Compounds on Breast Models

| Compound | HCC1419 | ZR-75-30 | UACC-812 |
|---|---|---|---|
| Enhertu (DAR8) | 0.356 | >1 | 0.263 |
| Trastuzumab-mono-L1-P5 | 0.193 | >5 | n/a |
| Trastuzumab-mono-L1-P7 | 0.555 | 0.985 | n/a |
| Trastuzumab-P5-L12-P7 | 0.238 | 0.850 | n/a |
| Disitamab-mono-L1-P5 | n/a | n/a | >50 |
| Disitamab-mono-L3-P8 | n/a | n/a | <5 |
| Disitamab-P5-L12-P7 | n/a | n/a | 0.191 |

*n/a: Conjugates not tested against these cell lines

TABLE 20
BCLxLi/BCL2i Dual ADC Cytotoxicity on a Multiple Myeloma Model
IC50 (nM) of Compounds on a MM Model

| Compound | KMS-27 |
|---|---|
| NY920-P5-L12-P4 | 0.022 |
| NY920-mono-L1-P4 | 0.154 |
| NY920-mono-L2-P5 | — |

TABLE 21
BCLxLi/BCL2i dual ADC Cytotoxicity on an Acute Myeloid
Leukemia Model
IC50 (nM) of Compounds on an AML Model

| Compound | EOL-1 |
|---|---|
| VHmil x VK1aNQ-P5-L12-P4 | 0.014 |
| VHmil x VK1aNQ-mono-L1-P4 | 0.042 |
| VHmil x VK1aNQ-mono-L2-P5 | >50 |

### Example 14. Synthesis and Characterization of anti-MET-Bcl-xLi ADCs DAR8

Exemplary antibody-drug conjugates (ADCs) were synthesized using the exemplary methods described below.

### Abbreviations

Ab antibody
ADC antibody-drug conjugate
CV column volume
DAR drug-to-antibody ratio
ESI electrospray ionisation
FA formic acid
LC-MS liquid chromatography mass spectrometry
L/P linker-payload
mAb monoclonal antibody
PBS phosphate buffer saline
PES polyether sulfone
PLRP-s polymeric reverse phase column
rmp reduction modifiable protein
SEC size exclusion chromatography
UPLC ultra-performance liquid chromatography
   Conjugation and analytical characterization of anti-MET-Bcl-xLi ADCs DAR8
   1. Antibodies specifications

Exemplary antibody-drug conjugates (ADCs) were synthesized using the exemplary methods described below. All antibodies used for the preparation of the exemplary ADCs were defined respectively by the abbreviation summarized in Table 22.

TABLE 22
Antibodies used for the synthesis of the exemplified ADC

| | | Mutations* | |
|---|---|---|---|
| Antibody | | (engineered | Origin of |
| abbreviation | Antibody | cysteines) | sequence |
| Ab Ma | MET_9006_IgG1 | E153C S376C | WO2016/042412 |
| Ab Mb | MET_9338_IgG1 | E149C S372C | WO2016/042412 |
| Ab Mc | MET_9006_IgG2 | E153C S372C | WO2016/042412 |
| Ab Md | MET_9338_IgG2 | E149C S368C | WO2016/042412 |
| Ab Mf | MET_8902_IgG1 | E153C S376C | |
| Ab Mg | MET_8902_IgG2 | E153C S372C | |
| Ab G1 | anti-chicken | E163C S386C | |
| | lysozyme | | |
| | MOR_IgG1 | | |
| Ab G2 | anti-chicken | E163C S382C | |
| | lysozyme | | |
| | MOR_IgG2 | | |

*The 2 engineered cysteines, namely E152C and S375C (the amino acid positions corresponding to 152 and 375 in a wild-type (unmodified) IgG1 heavy chain constant domain numbered according to EU numbering system, reference sequence from WO2015138615), were conserved in all the tested antibodies. The exact position of these substitutions is reported in the table above.

The exemplified ADCs were synthesised using site-specific conjugation. The antibodies Ab Ma, Ab Mb, Ab Mc, Ab Md, Ab Mf, Ab Mg, Ab Gi and Ab G2 were endowed with cysteine mutations incorporated inside the heavy chain and used to conjugate linker-payloads via maleimide group using the method M1 or M2.

### 2. Conjugation

The exemplified ADCs were synthesized using the linker-payload P5-L12-P5. General preparation of the antibody for site-specific cysteine conjugation: The conjugations were performed in a range of 5 mg antibody. The mAb was bound on rmp Protein A resin (GE Healthcare) at a ratio of 10 mg Ab to 1 ml resin in PBS by mixing in Biorad sized disposable column for 30 minutes. To deblock the reactive cysteines, cysteine hydrochloride monohydrate was added to a final concentration of 20 mM. The mixture was agitated at room temperature for 30 minutes followed by the washing of the resin with 5×50 CV of PBS on a vacuum manifold. The resin was then resuspended in an equal volume of PBS containing 250 nM of CuCl₂ and incubated for 1.5 h. Then the conjugation method M1 or M2 were used for the attachment of the linker-payload.

Conjugation method MI:

The re-oxidized antibody attached to protein A, was washed 5×50 CV of PBS on a vacuum manifold and resuspended in an equal resin volume of PBS. To the mixture were added 10-fold molar excess of a 10 mM solution of linker-payload and equal volume of DMF. The reaction was incubated at room temperature for 2 h. To monitor the conjugation 20 μl of resin slurry were removed, centrifuged, and after the supernatant was removed, the resin was eluted with 40 μl of the antibody elution buffer (Thermo Fisher Scientific) and analysed by PRLP-s. After eliminating the excess of linker-payload by washing the resin 5×50 CV with 5% DMF in PBS followed by 5×50CV PBS on a vacuum manifold, the ADC was eluted from protein A with the antibody elution buffer and was buffer exchanged by dialysis overnight (Thermo Fisher, 88254) in PBS 1X pH 7.4 (Sigma Life Science, P3813, 10PAK). Exemplified ADCs by method M1 were purified by SEC column HiLoad® 26/600 Superdex® 200 prep grade in 100% PBS.

Conjugation method 12:

The re-oxidized antibody attached to protein A, was washed 5×50 CV of PBS on a vacuum manifold and resuspended in an equal resin volume of PBS. To the mixture were added 10-fold molar excess of a 10 mM solution of linker-payload and equal volume of DMF. The reaction was incubated at room temperature for 2 h. To monitor the conjugation 20 μl of resin slurry were removed, centrifuged, and after the supernatant was removed, the resin was eluted with 40 μl of the antibody elution buffer (Thermo Fisher Scientific) and analysed by PRLP-s. After eliminating the excess of linker-payload by washing the resin 5×50 CV with 5% DMF in PBS followed by 5×50CV PBS on a vacuum manifold, the ADC was eluted from protein A with the antibody elution buffer and was buffer exchanged by dialysis overnight (Thermo Fisher, 88254) in PBS 1X pH 7.4 (Sigma Life Science, P3813, 10PAK).

All exemplified ADCs were directly concentrated using Vivaspin 20, 50KD, PES (Sartorius Stedim, VS2031), filtered sterilely through 0.2 μm sterile PES Filter, 25 mm (Whatmann, G896-2502) and stored at 4 ° C. All ADCs were characterized by analytical size exclusion chromatography Superdex 200 Increase 5/150 GL (GE Healthcare, 28990945) to determine monomer percentage and LC-MS for DAR determination.

To monitor the conjugation, reverse phase chromatography using an Agilent PLRP-S column 4000A 5 μm, 4.6×50 mm column (Buffer A water, 0.1% TFA, Buffer B Acetonitrile, 0.1% TFA, column held at 80° C., Flowrate 1.5 ml/min) was used.

### 3. Characterization

### LC-MS General Methodology

Drug-to-antibody ratio (DAR) of the exemplary ADCs was determined by liquid chromatography hyphenated with mass spectrometry (LC-MS) using the following method:

### LC-MS 1 (80% Phase A (Water/0.1% FA), 20% Phase B (Acetonitrile/0.1% FA)):

ADC was loaded onto a Bioresolve RP mAb Polyphenyl,column 450A, 2.7 μm, 2.1*150 mm (Waters, Saint-Quentin-en-Yvelines, France, 186008946). For analysis in both intact and reduced conditions, a desalting step was performed for 1.5 min at 20% of B with a flow rate of 0.6 mL/min. Elution step was performed with a gradient from 1.5 min at 20% B to 16.5 min at 50% B with a flow rate of 0.6 mL/min. A wash step was set from 16.8 min to 18.8 min at 100% B with a flow rate of 0.6 mL/min. Finally, a conditioning step was used at 19.2 min for 1.8 min at 20% B with a flow rate of 0.6 mL/min (Total run time=21 min). For this method, mobile phase A was ultrapure water obtained with Mili-Q® system and mobile phase B was MS grade acetonitrile (Biosolve, Dieuze, France, 0001204101BS) supplemented with 0.1% of FA (Fisher Chemical: A117-50-50ML). Column temperature was set at 80° C. A general MS method was optimized for all synthesized ADCs in order to determine average DAR (Table 23).

LC-MS analysis was performed using a Waters UPLC H-Class Bio chromatography system hyphenated with a Q-TOF Synapt G2 S ESI mass spectrometer (Waters, Manchester, UK). The ADC was either analysed in intact condition with a deglycosylation step using PNGase F enzyme (Genovis®, G1-PF1-010) or following reduction with 5 mM (final concentration) of dithiothreitol DTT (Thermo Scientific, Rockford, IL, 20291). Subsequently, the treated ADC was analysed using the above-mentioned LC (Table 23). Electrospray-ionization time-of-flight mass spectra of the analytes were acquired using UNIFI™ acquisition software (Waters, Manchester, UK). Then, the extracted intensity vs. m/z spectrum was deconvoluted using Maximum Entropy (MaxEntl) method of MassLynx™ software in order to determine the mass of each intact antibody species or each reduced antibody fragment depending on the treatment. Finally, DAR was determined from the deconvoluted spectra by summing the integrated MS (total ion current) peak area of unconjugated and conjugated given species (mAb or associated fragment). For the DAR determination, the percentage of each specie identified was calculated by intensity peak value from deconvoluted spectra. The percentage obtained, was multiplied by the number of drugs attached. The summed results produced an estimation of the final average DAR value for the full ADC*2.

### Size Exclusion Chromatography

Size exclusion chromatography (SEC) was performed for the quality control of each ADCs by measuring monomer percentage of the conjugate. The analysis was performed on analytical column Superdex 200 Increase 5/150 GL (GE Healthcare, 28990945) in isocratic conditions 100% PBS pH7.4 (Sigma Life Science, P3813, 1OPAK), flow 0.45 m/min for 12 minutes. The % aggregate fraction of the conjugate sample was quantified based on the peak area absorbance at 280 nm. Its calculation was based on the ratio between the high molecular weight eluent at 280 nm divided by the sum of peak area absorbance at the same wavelength of the high molecular weight and monomeric eluents multiplied by 100.

### 4. Results

Characterization of the exemplary ADCs was summarized in Table 23 (coupling, LC-MS method, DAR and aggregation status). The average DAR values were determined using the above LC-MS methods and the percentage of aggregates was measured by size exclusion chromatography (SEC) during the quality control of the ADC.

The following exemplified ADCs are prepared using the procedures described above (Table 23).

TABLE 23
ADC analytical characterization and coupling methodology

| | Coupling | LC-MS | DAR | % Agg |
|---|---|---|---|---|
| ADC | Method | Method | (LC-MS) | (SEC) |
| Ab Ma - P5-L12-P5 | M2 | LC-MS 1 | 5.9 | 2 |
| Ab Mc - P5-L12-P5 | M1 | LC-MS 1 | 6.2 | 3 |
| Ab Mb - P5-L12-P5 | M2 | LC-MS 1 | 5.4 | 2 |
| Ab Md - P5-L12-P5 | M1 | LC-MS 1 | 6.3 | 2 |
| Ab Mf - P5-L12-P5 | M2 | LC-MS 1 | 6.1 | 4 |
| Ab Mg - P5-L12-P5 | M2 | LC-MS 1 | 5.5 | 5 |
| Ab G1 - P5-L12-P5 | M1 | LC-MS 1 | 6.3 | 6 |
| Ab G2 - P5-L12-P5 | M1 | LC-MS 1 | 4.7 | 2 |

1. An antibody-drug conjugate comprising an antibody or an antigen-binding fragment thereof covalently linked to two antineoplastic payloads through a dual linker, wherein at least one antineoplastic payload is a BH3 mimetic, and wherein the dual linker has one attachment point connected to the antibody and two attachment points to the two antineoplastic payloads and wherein the two antineoplastic payloads can be the same or different.

2. (canceled)

3. The antibody-drug conjugate of claim 1, wherein one antineoplastic payload is a BH3 mimetic and the other antineoplastic payload is an antineoplastic non-BH3 mimetic and the antineoplastic non-BH3 mimetic is a topoisomerase 1 inhibitor or an anti-mitotic drug; optionally wherein:
(i) the topoisomerase 1 inhibitor is selected from topotecan, exatecan, deruxtecan and SN-38; and
(ii) the anti-mitotic drug is monomethyl auristatin E (MMAE) or a taxane; preferably wherein the taxane is docetaxel, paclitaxel, or cabazitaxel.

4-6. (canceled)

7. The antibody-drug conjugate of claim 1, wherein:
ii said two antineoplastic payloads are two BH3 mimetics;
(ii) said two antineoplastic payloads are two BH3 mimetics and the BH3 mimetic is selected from a Mcl-1 inhibitor, a Bcl-2 inhibitor, and a Bcl-xL inhibitor;
(iii) the BH3 mimetics of said two antineoplastic payloads are the same;
(iv) the BH3 mimetics of said two antineoplastic payloads are different;
(v) one antineoplastic payload is a Mcl-1 inhibitor and the other antineoplastic payload is a Bcl-2 inhibitor;
(vi) one antineoplastic payload is a Mcl-1 inhibitor and the other antineoplastic payload is a Bcl-xL inhibitor; or
(vii) one antineoplastic payload is a Bcl-2 inhibitor and the other antineoplastic payload is a Bcl-xL inhibitor.

8-11. (canceled)

12. The antibody-drug conjugate of claim 1, wherein:
one antineoplastic payload is a Mcl-1 inhibitor, a Bcl-2 inhibitor, and a Bcl-xL inhibitor, and the other antineoplastic payload is a topoisomerase 1 inhibitor or an anti-mitotic drug;
(ii) one antineoplastic payload is a Bcl-xL inhibitor and the other antineoplastic payload is a topoisomerase 1 inhibitor;
(iii) one antineoplastic payload is a Bcl-xL inhibitor and the other antineoplastic payload is an anti-mitotic drug;
(iv) one antineoplastic payload is a Mcl-1 inhibitor and the other antineoplastic payload is a topoisomerase 1 inhibitor;
(v) one antineoplastic payload is a Mcl-1 inhibitor and the other antineoplastic payload is an anti-mitotic drug;
(vi) one antineoplastic payload is a Bcl-2 inhibitor and the other antineoplastic payload is a topoisomerase 1 inhibitor; or
(vii) one antineoplastic payload is a Bcl-2 inhibitor and the other antineoplastic payload is an anti-mitotic drug.

13-18. (canceled)

19. The antibody-drug conjugate of claim 1, wherein the antibody-drug conjugate is represented by Formula (A): 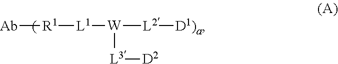
wherein:
Ab is an antibody or an antigen-binding fragment thereof;
R¹ is an attachment group;
L¹ is a bridging spacer;
W is branching moiety;
L^{2′} and L^{3′}, are each independently a linker;
D¹ and D² are each independently an antineoplastic payload, wherein at least one of Di and D² is a BH3 mimetic; and
a is an integer from 1 to 16, optionally, wherein:
(i) D¹ and D² are each independently a BH3 mimetic;
(ii) a is an integer from 1 to 6 or from 1 to 4 or a is 1 or 2 or a is determined by liquid chromatography-mass spectrometry (LC-MS); and/or
(iii) each of L²′ and L³′ comprises a cleavable group, optionally wherein:
(a) at least one cleavable group comprises a glucuronide group, pyrophosphate group, a peptide group, and/or a self-immolative group; or
(b) at least one cleavable group comprises a pyrophosphate group, a peptide group, and/or a self-immolative group.

20-23. (canceled)

24. The antibody-drug conjugate of claim 1, wherein the antibody-drug conjugate is represented by Formula (B): 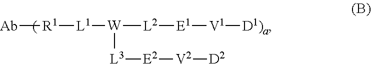
wherein:
Ab is an antibody or an antigen-binding fragment thereof;
R¹ is an attachment group;
L¹ is a bridging spacer;
W is N or CR^{w}; wherein R^{W} is H or C₁₋₆alkyl;
L² and L³ are each independently a connecting spacer;
E¹ and E² are each independently an enzyme cleavage element or a hydrophilic moiety;
V¹ and V² each independently comprise i) a self immolative group, ii) an enzyme cleavage element, or iii) a self immolative group and an enzyme cleavage element;
D¹ and D² are each independently an antineoplastic payload, wherein at least one of D¹ and D² is a BH3 mimetic; and
a is an integer from 1 to 16, optionally, wherein:.
(i) V¹ and V² are each independently (a) a self immolative group or (b) an enzyme cleavage element; and D¹ and D² are each independently a BH3 mimetic;
(ii) V¹ and V² each independently comprises a phosphate, a pyrophosphate and/or a self-immolative group;
(iii) V¹ and V² each independently comprises a self-immolative group;
(iv) V¹ and V² each independently comprises a self-immolative group comprising —CH₂—O—, —OC(═O)—, —NH—CH₂—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium; or
(v) V¹ and V² each independently comprises a group comprising para-aminobenzyl-phosphate or para-aminobenzyl-pyrophosphate.

25-27. (canceled)

28. The antibody-drug conjugate of claim 1, wherein the antibody-drug conjugate is represented by Formula (C): 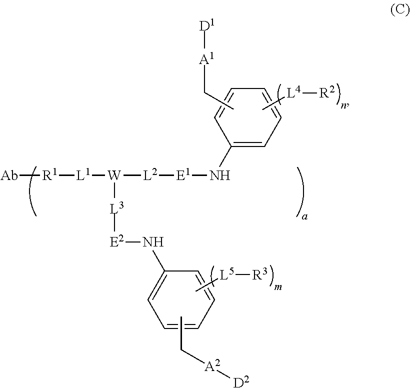
or pharmaceutically acceptable salt thereof, wherein
Ab is an antibody or an antigen-binding fragment thereof;
R¹ is an attachment group;
L¹ is a bridging spacer;
W is N or CR^{w}; wherein R^{W} is H or C₁₋₆alkyl;
L² and L³ are each independently a connecting spacer;
E¹ and E² are each independently a peptide group comprising 1 to 6 amino acids, wherein said peptide group is optionally substituted by a hydrophilic group;
A¹ and A² are each independently a bond, —OC(═O)—*, —OC(═O)NH—*, 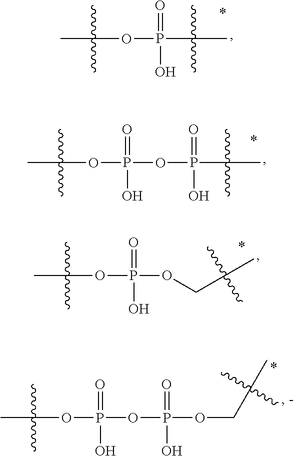 [SMILES: *OP(*)(=O)O ; *OP(=O)(O)OP(*)(=O)O ; *COP(=O)(O)O* ; *COP(=O)(O)OP(=O)(O)O*]
OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A¹ or A² indicates the point of attachment to D¹ or D² D¹ and D² are each independently an antineoplastic payload, wherein at least one of D¹ and D² is a BH3 mimetic; preferably wherein D1 and D2 are each independently a BH3 mimetic;
L⁴ and L⁵ are each independently a spacer moiety;
R² and R³ are each independently a hydrophilic group or an enzyme cleavage element;
m and n are each independently 0 or 1; and
a is an integer from 1 to 16.

29. (canceled)

30. The antibody-drug conjugate of claim 1, wherein the antibody-drug conjugate is represented by Formula (D1), (D2), or (D3): 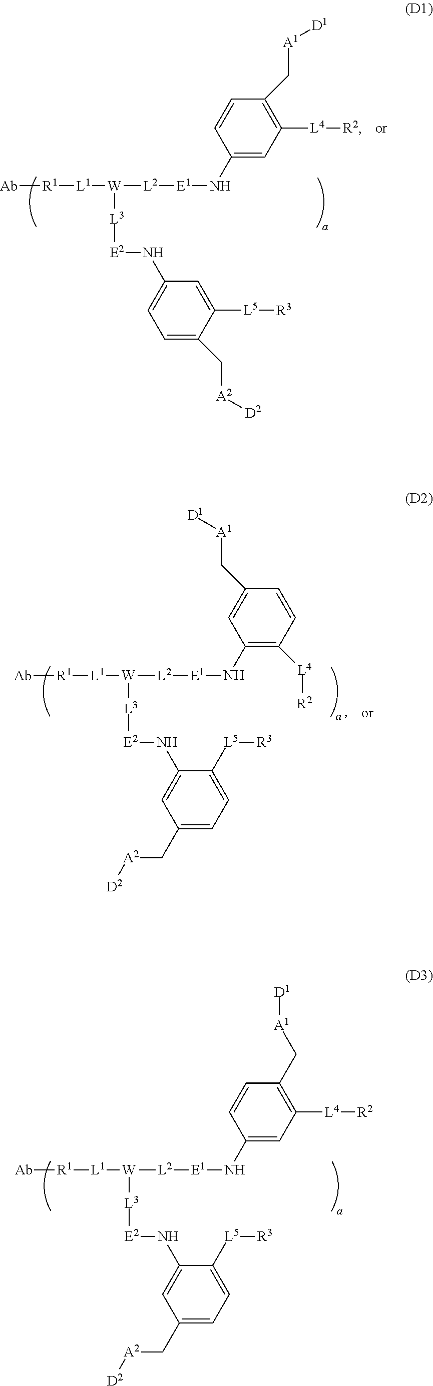
or pharmaceutically acceptable salt thereof, wherein D¹ and D² are each independently an antineoplastic payload, wherein at least one of D¹ and D² is a BH3 mimetic; for Formula (D2), R² and R³ are each independently an enzyme cleavage element; and for Formula (D3), R² is a hydrophilic group and R³ is an enzyme cleavage element; preferably wherein D¹ and D² are each independently a BH3 mimetic; optionally wherein for Formula (D1), R² and R³ are each independently a hydrophilic group.

31-32. (canceled)

33. The antibody-drug conjugate of claim 19, wherein:
(i) a is an integer from 1 to 8, 1 to 6, 1 to 4, or a is 1 or 2, optionally wherein a is determined by liquid chromatography-mass spectrometry (LC-MS);
(ii) the attachment group is formed by a reaction comprising at least one reactive group;
(iii) the attachment group is formed by reacting:
a first reactive group that is attached to the linker, and
a second reactive group that is attached to the antibody or is an amino acid residue of the antibody, wherein optionally,
(a) at least one of the reactive groups comprises:
a thiol,
a maleimide,
a haloacetamide,
an azide,
an alkyne,
a cyclooctene,
a triaryl phosphine,
an oxanorbornadiene,
a cyclooctyne,
a diaryl tetrazine,
a monoaryl tetrazine,
a norbornene,
an aldehyde,
a hydroxylamine,
a hydrazine,
NH₂—NH—C(═O)—,
a ketone,
a vinyl sulfone,
an aziridine,
an amino acid residue, 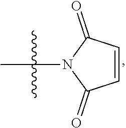 [SMILES: *N1C(=O)C=CC1=O]
—ONH₂, —NH₂, 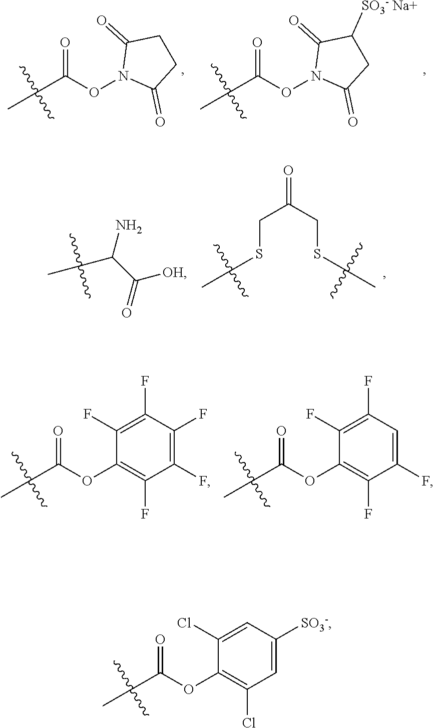
—SSR¹², —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, 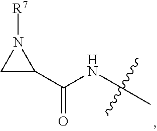
—C(O)NHNH₂, 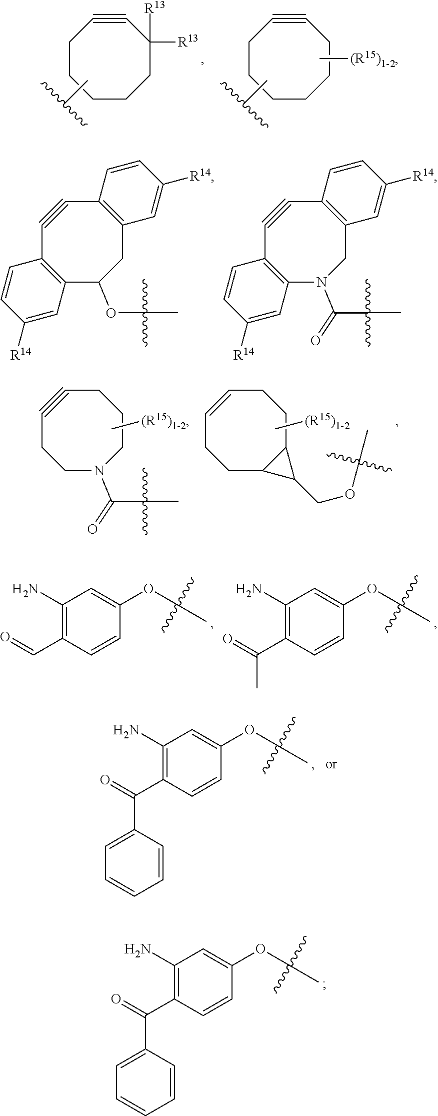
wherein:
each R¹¹ is independently selected from H and C₁-C₆alkyl;
each R¹² is 2-pyridyl or 4-pyridyl;
each R¹³ is independently selected from H, C₁-C₆alkyl, F, Cl, and —OH:
each R¹⁴ is independently selected from H, C₁-C₆alkyl, F, Cl, —NH₂, —OCH₃, —OCH₂CH₃, —N(CH₃)₂, —CN, —NO₂ and —OH:
each R¹⁵ is independently selected from H, C₁₋₆alkyl, fluoro, benzyloxy substituted with —C(═O)OH, benzyl substituted with —C(═O)OH, C₁₋4alkoxy substituted with —C(═O)OH and C₁₋₄ alkyl substituted with —C(═O)OH; and/or
(b) the first reactive group and second reactive group comprise:
a thiol and a maleimide,
a thiol and a haloacetamide,
a thiol and a vinyl sulfone,
a thiol and an aziridine,
an azide and an alkyne,
an azide and a cyclooctyne,
an azide and a cyclooctene,
an azide and a triaryl phosphine,
an azide and an oxanorbornadiene,
a diaryl tetrazine and a cyclooctene,
a monoaryl tetrazine and a norbornene,
an aldehyde and a hydroxylamine,
an aldehyde and a hydrazine,
an aldehyde and NH₂—NH—C(═O)—,
a ketone and a hydroxylamine,
a ketone and a hydrazine,
a ketone and NH₂—NH—C(═O)—,
a hydroxylamine and 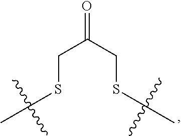 [SMILES: *SCC(=O)CS*]
an amine and 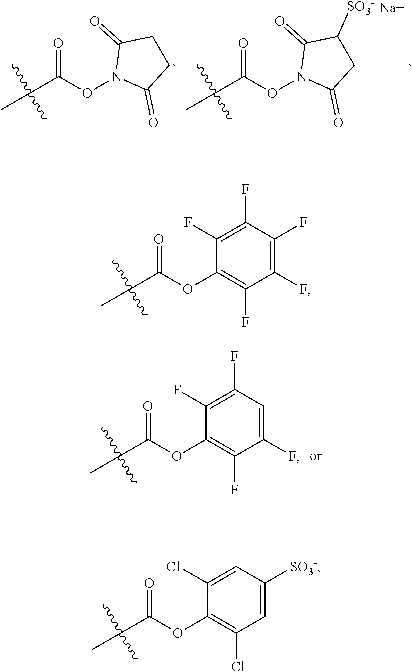
or a CoA or CoA analogue and a serine residue;
(iv) the attachment group is selected from: 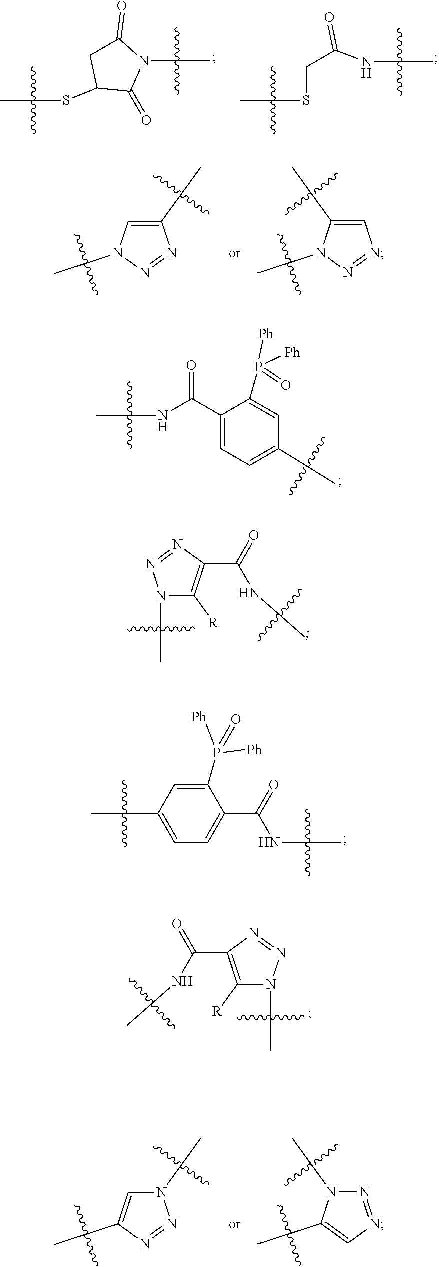 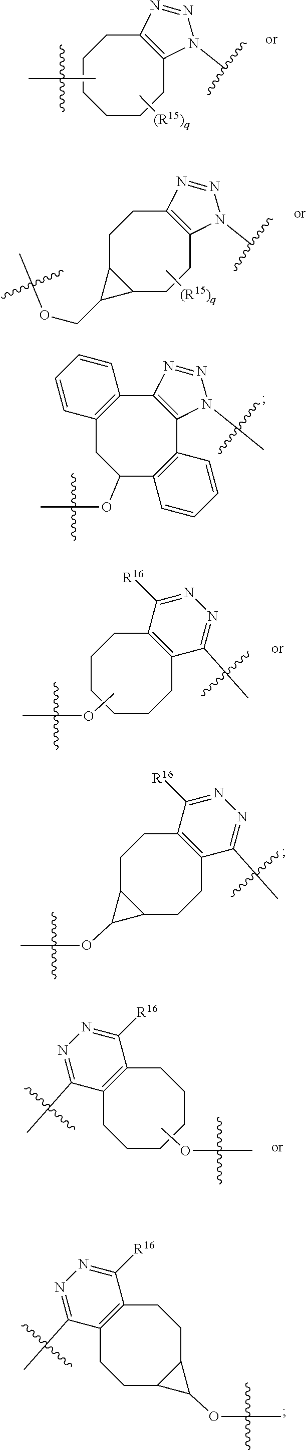 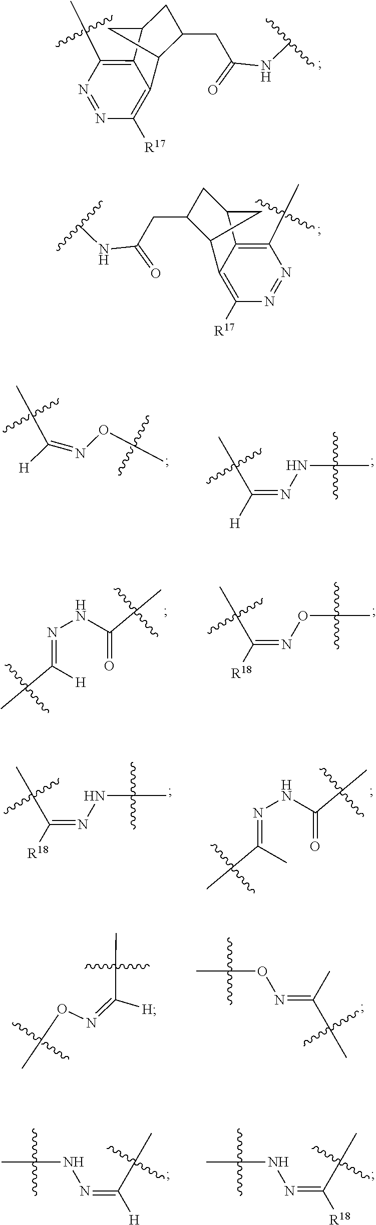 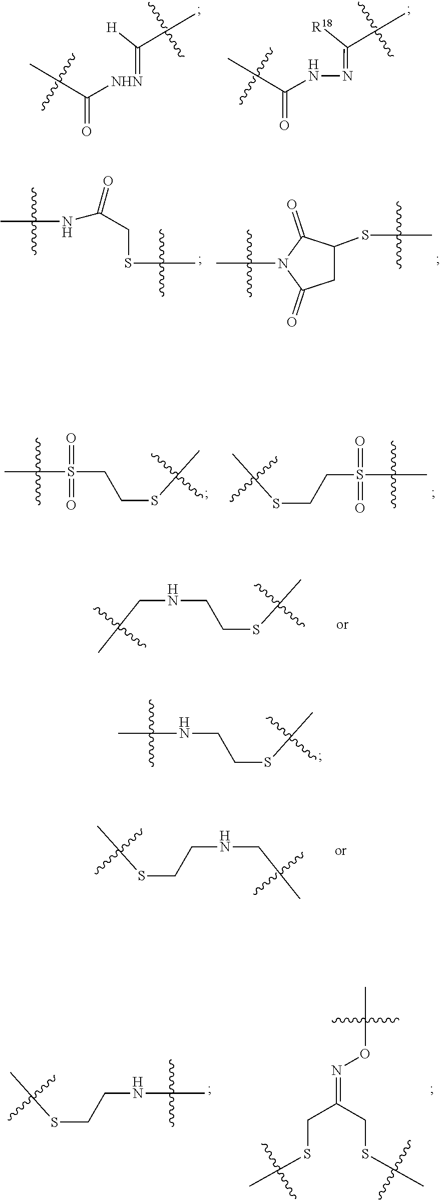
amide; 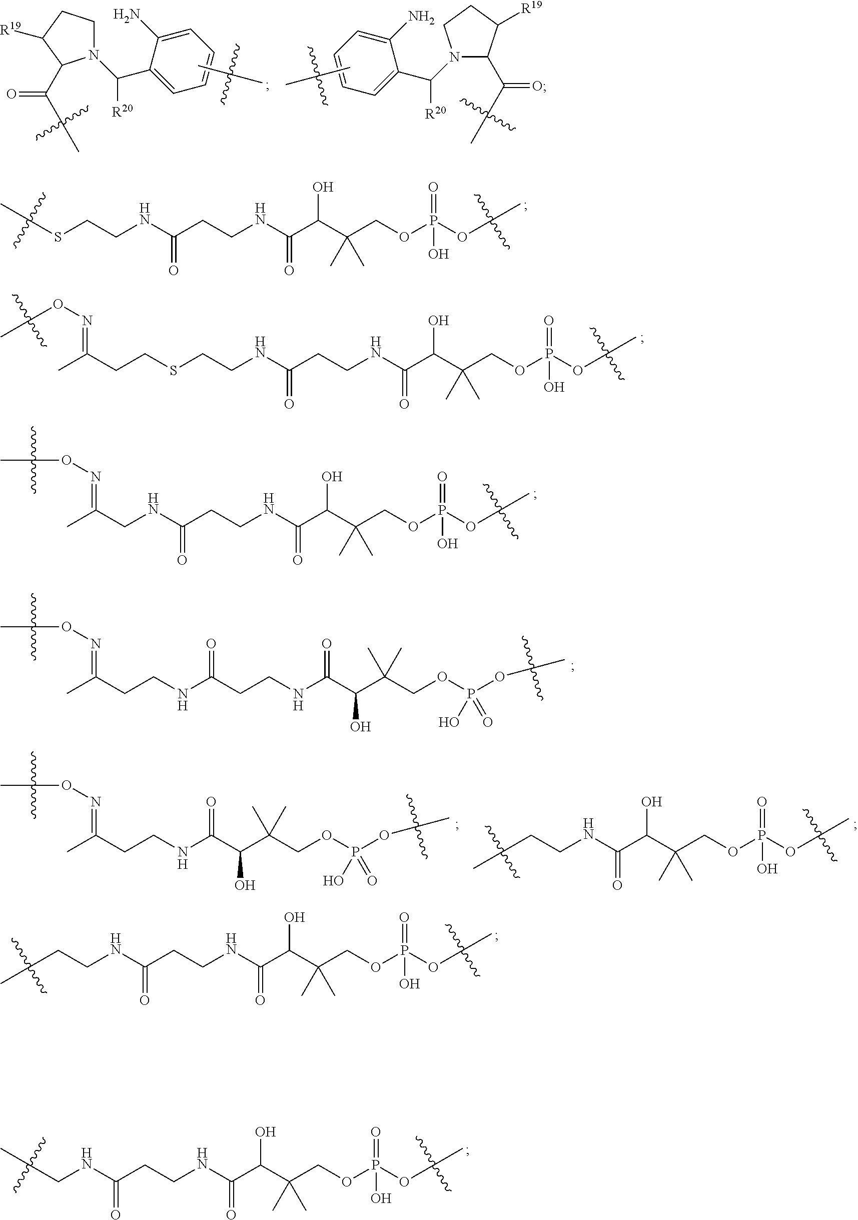
and
disulfide,
wherein:
R¹⁶ is H, C₁0.4 alkyl, phenyl, pyrimidine or pyridine;
R¹⁸ is H, C₁0.6 alkyl, phenyl or C₁0.4 alkyl substituted with 1 to 3—OH groups;
each R¹⁵ is independently selected from H, C₁0.6 alkyl, fluoro, benzyloxy substituted with —C(═O)OH, benzyl substituted with —C(═O)OH, C₁0.4 alkoxy substituted with —C(═O)OH and C₁0.4 alkyl substituted with —C(═O)OH:
R¹⁷ is independently selected from H, phenyl and pyridine;
q is 0, 1,2 or 3;
R¹⁹ is H or methyl; and
R²⁰ is H, —CH₃ or phenyl; or
(v) the attachment group is

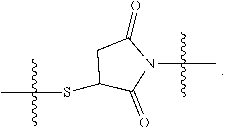 [SMILES: *SC1CC(=O)N(*)C1=O]

34-37. (canceled)

38. The antibody-drug conjugate of claim 19 wherein:
(1) L¹ comprises: 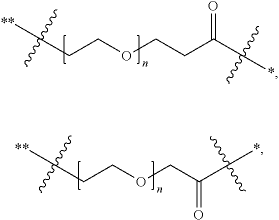
or
*—CH(OH)CH(OH)CH(OH)CH(OH)—**,
wherein each n is an integer from 1 to 12, wherein the * of L¹ indicates the point of direct or indirect attachment to W, and the ** of ^{L} indicates the point of direct or indirect attachment to R¹;
(2) L¹ is 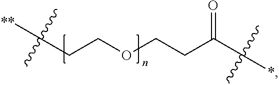
and n is an integer from 1 to 12 or n is 1 or n is 12, wherein the * of L¹ indicates the point of direct or indirect attachment to W, and the ** of Li indicates the point of direct or indirect attachment to R¹;
(3) L¹ is 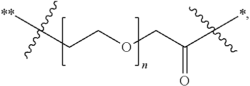
and n is an integer from 1 to 12, wherein the * of Li indicates the point of direct or indirect attachment to W, and the ** of Li indicates the point of direct or indirect attachment to Ri;
(4) L¹ comprises 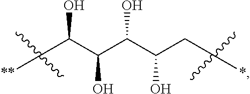
wherein the * of L¹ indicates the point of direct or indirect attachment to W, and the ** of L¹ indicates the point of direct or indirect attachment to R¹;
(5) L¹ is a bridging spacer comprising:
*—C(═O)(CH₂)ₘO(CH₂)ₘ—**; *—C(═O)((CH₂)ₘO)ₜ(CH₂)ₙ—**; *—C(═O)(CH₂)ₘ—**;
*—C(═O)NH((CH₂)ₘO)ₜ(CH₂)ₙ—**;
*—C(═O)O(CH₂)ₘSSC(R^{L1})₂(CH₂)ₘC(═O)NR^{L1}(CH₂)ₘNR^{L1}C(═O)(CH₂)ₘ—**;
*—C(═O)O(CH₂)ₘC(═O)NH(CH₂)ₘ—**; *—C(═O)(CH₂)ₘNH(CH₂)ₘ—**;
*—C(═O)(CH₂)ₘNH(CH₂),C(═O)—**; *—C(═O)(CH₂)ₘX₁(CH₂)ₘ—**;
*—C(═O)((CH₂)ₘO)ₜ(CH₂).X(CH₂)ₙ—**; *—C(═O)(CH₂)ₘNHC(═O)(CH₂)ₙ—**;
*—C(═O)((CH₂)ₘO)ₜ(CH₂),NHC(═O)(CH₂)ₙ—**;
*—C(═O)(CH₂)ₘNHC(═O)(CH₂),XI(CH₂)ₙ—**;
*—C(═O)((CH₂)ₘO)ₜ(CH₂),NHC(═O)(CH₂),X(CH₂)ₙ—**;
*—C(═O)((CH₂)ₘO)ₜ(CH₂),C(═O)NH(CH₂)ₘ—**; *—C(═O)(CH₂)ₘC(R^{L1})₂—** or
—C(═O)(CH₂)ₘC(═O)NH(CH₂)ₘ—**, wherein the * of L¹ indicates the point of direct or indirect attachment to W, and the ** of L¹ indicates the point of direct or indirect attachment to R¹;
X₁ is 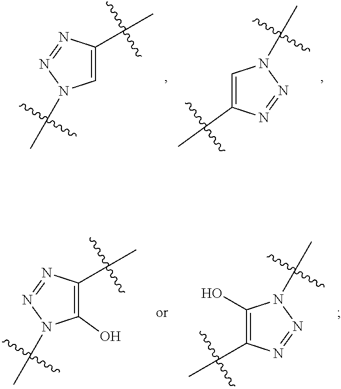 [SMILES: *c1cn(*)nn1 ; *c1cn(*)nn1 ; *c1nnn(*)c1O ; *c1nnn(*)c1O]
and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30; and
each R^{L1} is independently selected from H and C₁-C₆alkyl;
(6) L¹ comprises a moiety represented by 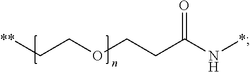
wherein n is an integer from 1 to 12, wherein the * of Li indicates the point of direct or indirect attachment to W, and the ** of L indicates the point of direct or indirect attachment to
(7) L¹ is represented by a formula 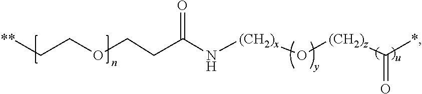
wherein
n is an integer from 1 to 12;
x is an integer from 0 to 6;
y is 0 or 1;
z is an integer from 0 to 6;
u or 1; and
wherein the * of Li indicates the point of direct attachment to W, and the ** of L indicates the point of direct attachment to R¹; or
(8) L¹ is selected from the group consisting of:

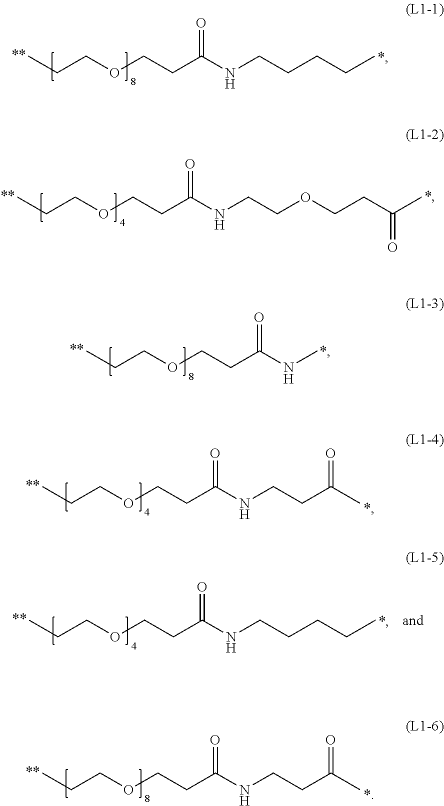

39-41. (canceled)

42. The antibody-drug conjugate of claim 24, wherein:
(i) L² and L³ are each independently a connecting spacer comprising a moiety represented by: 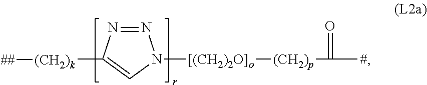
wherein
k is an integer from 0 to 6;
r is 0 or 1;
is an integer from 0 to 12;
p is an integer from 0 to 6; and
wherein the #of L² or L³ indicates the point of direct or indirect attachment to E¹ or E2 respectively, and the ##of L² or L³ indicates the point of direct or indirect attachment to W;
(ii) L² and L³ are each independently a connecting spacer selected from a group consisting of 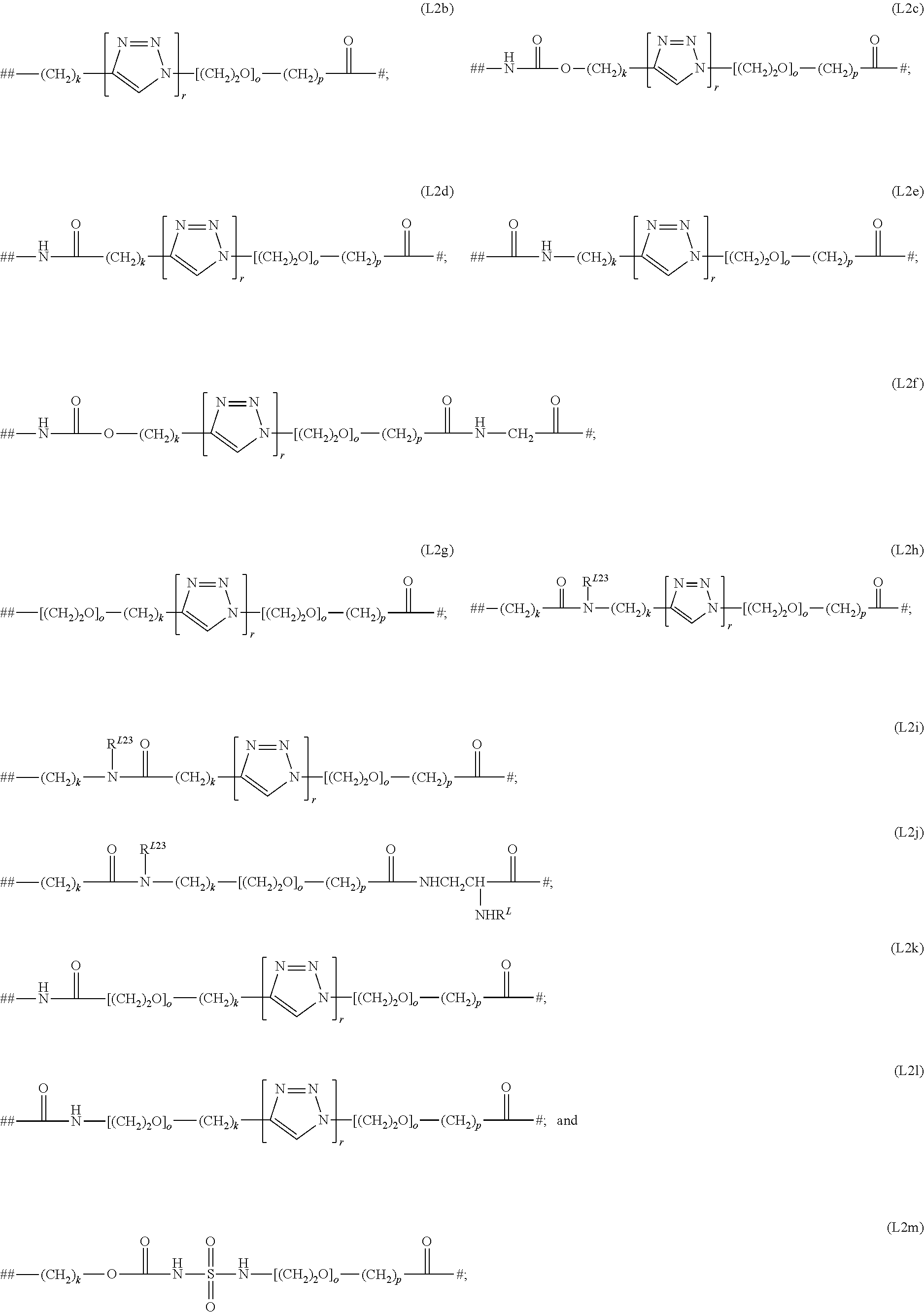
wherein
k, in each occurrence, is independently an integer from 0 to 4;
r, in each occurrence, is independently 0 or 1;
o, in each occurrence, is independently an integer from 0 to 10;
p, in each occurrence, is independently an integer from 0 to 4;
R^{L23} is hydrogen or C₁₋₆alkyl;
R^{L} is hydrogen or —C(O)—RH.
R^{H} is a hydrophilic group; and
the #of L² or L³ indicates the point of direct attachment to E¹ or E², respectively, and the ##of L² or L³ indicates the point of direct attachment to W;
provided that when W is N, L² and L³ are not (L2c), (L2d), (L2f), or (L2k);
(iii) L² and L³ are each independently a connecting spacer selected from a group consisting of 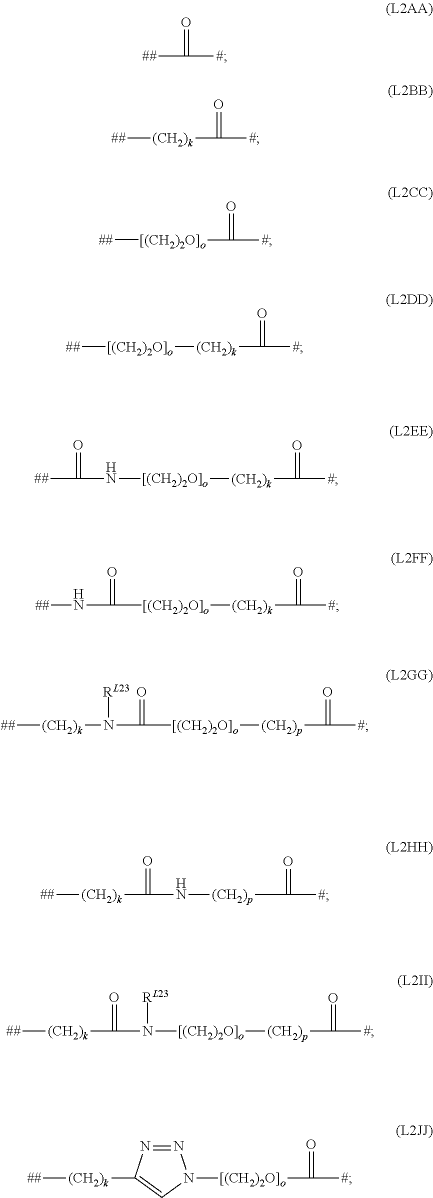 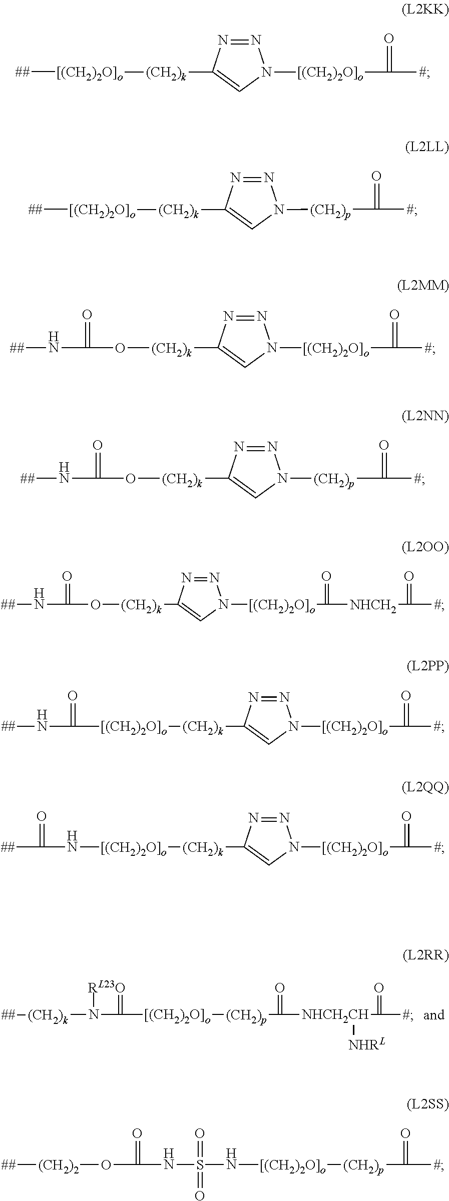
wherein
k, in each occurrence, is independently an integer from 1 to 3;
o, in each occurrence, is independently an integer from 1 to 9;
p, in each occurrence, is independently an integer from 1 to 3;
R^{L23} is hydrogen or C₁₋3alkyl;
R^{L} is hydrogen or —C(O)—RH.
R^{H} is a hydrophilic group; and
the #of L² or L³ indicates the point of direct attachment to E¹ or E², respectively, and the ##of L² or L³ indicates the point of direct attachment to W; provided that when W is N, L² and L³ are not (L2FF), (L2MM), (L2NN), (L200), or (L2PP); or
(iv) L² and L³, independently, are a connecting spacer selected from a group consisting of 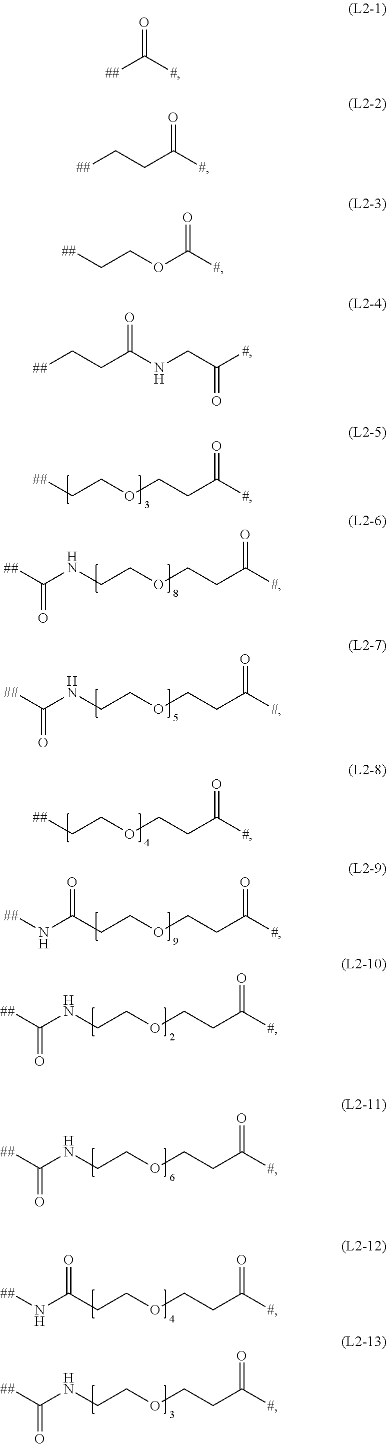 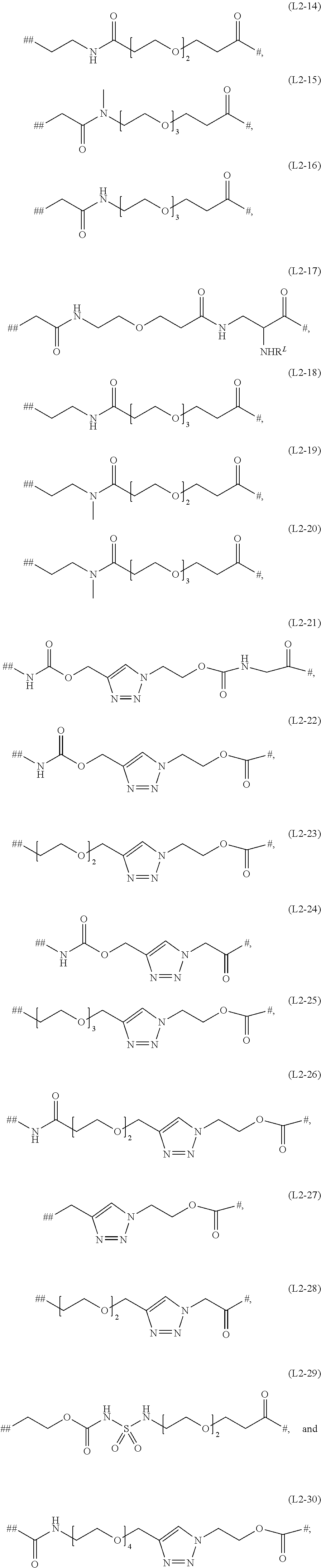
wherein the #of L² or L³ indicates the point of direct attachment to E¹ or E2, respectively, the ##of L² or L³ indicates the point of direct attachment to W; R^{L} is hydrogen or -C(O)—R^{H}; and
R^{H} is 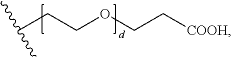
and d is an integer from 20 to 30 (e.g., 20, 21, 22, 23, 24, 25, 26, 27, 28, 29 or 30); optionally wherein d is 25.

43-46. (canceled)

47. The antibody-drug conjugate of claim 28, wherein:
ii the peptide group comprises 1 to 4, 1 to 3, or 1 to 2 amino acid residues; optionally wherein the amino acid residues are selected from glycine (Gly), L-valine (Val), L-citrulline (Cit), L-cysteic acid (sulfo-Ala), L-lysine (Lys), L-isoleucine (Ile), L-phenylalanine (Phe), L-methionine (Met), L-asparagine (Asn), L-proline (Pro), L-alanine (Ala), L-leucine (Leu), L-tryptophan (Trp), L-tyrosine (Tyr) and b-alanine (b-Ala);
(ii) the peptide group comprises Val-Cit, Phe-Lys, Val-Ala, Val-Lys, Leu-Cit, Cit-(P-Ala), Gly-Gly-Gly, Gly- Gly-Phe-Gly, and/or sulfo-Ala-Val-Ala;
(iii) the peptide group represented by E¹ or E² is an enzyme cleavage element; or
(iv) the peptide group represented by E¹ or E² is a hydrophilic moiety; optionally wherein
(a) E¹ or E², independently, is an enzyme cleavage element selected from a group consisting of 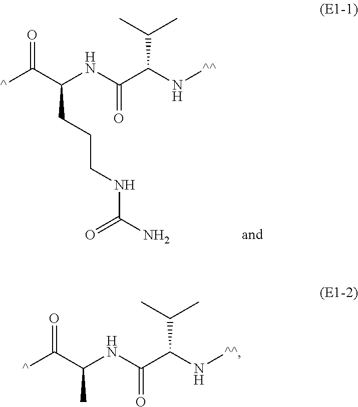
wherein \hat{ } of E¹ or E² indicates the point of direct attachment to V¹ or V² in Formula (B) or direct attachment to the -NH- group in Formula (C) and (D); and \hat{ }\hat{ } of E or E² indicates the point of direct attachment to L2 or L³, respectively; or
(b) E¹ or E², independently, is a hydrophilic moiety represented by 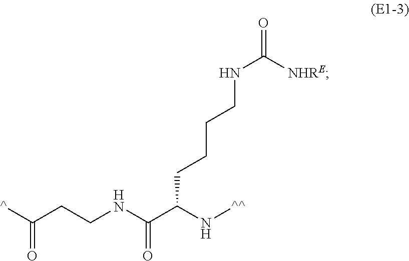
wherein R^{E} is a hydrophilic group R^{H}: preferably wherein each hydrophilic group R^{H} in E¹ or E² is independently 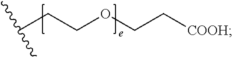
wherein e is an integer between 20 and (e.g., 20, 21, 22, 23, 24, 25, 26, 27, 28, 29 or 30); preferably wherein e is 24.

48-55. (canceled)

56. The antibody-drug conjugate of claim 28, or pharmaceutically acceptable salt thereof, wherein;
(i) A¹ and A² independently are a bond, —OC(═O)—*, or 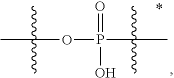 [SMILES: *OP(*)(=O)O]
wherein * indicates the point of attachment to D¹ or D²
(ii) A¹ and A² independently are a bond or 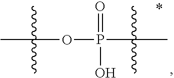 [SMILES: *OP(*)(=O)O]
wherein * indicates the point of attachment to D¹ or D²
(iii) A¹ and A² independently are a bond or —OC(═O)—*, wherein * indicates the point of attachment to D¹ or D²
(iv) Aⁱ and A² are —OC(═O)—*;
(v) A¹ and A² are 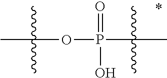 [SMILES: *OP(*)(=O)O]
(vi) A¹ is —OC(═O)—* and A² is a bond
(vii) A¹ is —OC(═O)—* and A² is 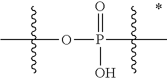 [SMILES: *OP(*)(=O)O]
(viii) A¹ is a bond and A² is 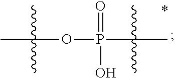 [SMILES: *OP(*)(=O)O]
(ix) A¹ is a bond and A² is —OC(═O)—*;
(x) A¹ and A² are a bond,
wherein * indicates the point of attachment to D¹ or D².

57-60. (canceled)

61. The antibody-drug conjugate of claim 28, wherein 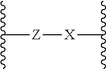
i) L⁴ and L⁵ are each independently a spacer moiety having the structure z-X wherein:
Z is —O—, —CH₂—, —CH₂O—, —CH₂N(R^{L5})C(═O)O—, —NHC(═O)C(R^{L5})₂NHC(═O)O—, —NHC(═O)C(R^{L5})₂NH—, —NHC(═O)C(R^{L5})₂NHC(═O)—, —C(═O)NR^{L45}—C(═O)NH—, —CH₂NR^{L45} C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈ cycloalkyl; and
X is a bond, triazolyl, or —CH₂-triazolyl-,
wherein X is connected to R2 or R³;
(ii) L⁴ and L⁵, independently, are a spacer moiety having the structure 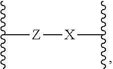
wherein:
Z is —CH₂—, —CH₂O—, —CH₂N(R^{L5})C(═O)O—, —NHC(═O)C(R^{L5})₂NHC(═O)O—, —NHC(═O)C(R^{L5})₂NH—, —NHC(═O)C(R^{L5})₂NHC(═O)—, —C(═O)NR^{b}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈ cycloalkyl; and
X is —CH₂-triazolyl-C₁₋₄ alkylene-OC(O)NHS(O)₂NH—, -C₄₋₆ cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂-triazolyl-C₁₋₄ alkylene-OC(O)NHS(O)₂NH—(CH₂CH₂O)ₙ—, —C₄₋₆cycloalkylene-OC(O)NHS(O)₂NH—(CH₂CH₂O)ₙ—, wherein each n independently is 1, 2, or 3, wherein X is connected to R2 or R³; preferably wherein Z is —O—, —CH₂NR^{L45} C(═O)—, —CH₂NR^{L45}C(═O)NH— or —CH₂O—; X is a bond, triazolyl, or —CH₂-triazolyl-; and R^{L45}, in each occurrence, is independently H or C₁₋3alkyl or
(iii) L⁴ and L⁵ are each independently a spacer moiety selected from a group consisting of 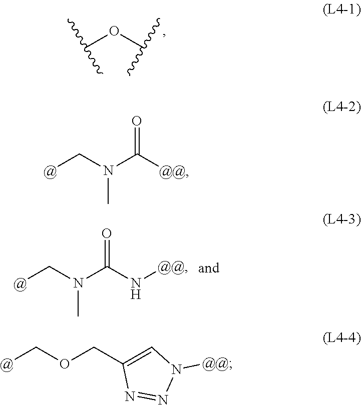
wherein the @of L⁴ or L⁵ indicates the point of direct attachment to the phenyl group, and the @@of L⁴ or L^{S} indicates the point of direct attachment to R² or R³.

62-63. (canceled)

64. The antibody-drug conjugate of claim 28, wherein:
(i) the hydrophilic groups represented by R² and R³ each independently comprises polyethylene glycol, polyalkylene glycol, a polyol, a polysarcosine, a sugar, an oligosaccharide, a polypeptide, C₂-C₆ alkyl substituted with 1 to 3 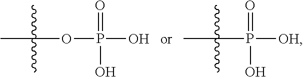 [SMILES: *OP(=O)(O)O ; *P(=O)(O)O]
or C₂-C₆alkyl substituted with 1 to 2 substituents independently selected from —OC(═O)NHS(O)₂NHCH₂CH₂OCH₃, —NHC(═O)C₁₋4alkylene-P(O)(OCH₂CH₃)₂ and —COOH groups;
(ii) R² or R³ independently is 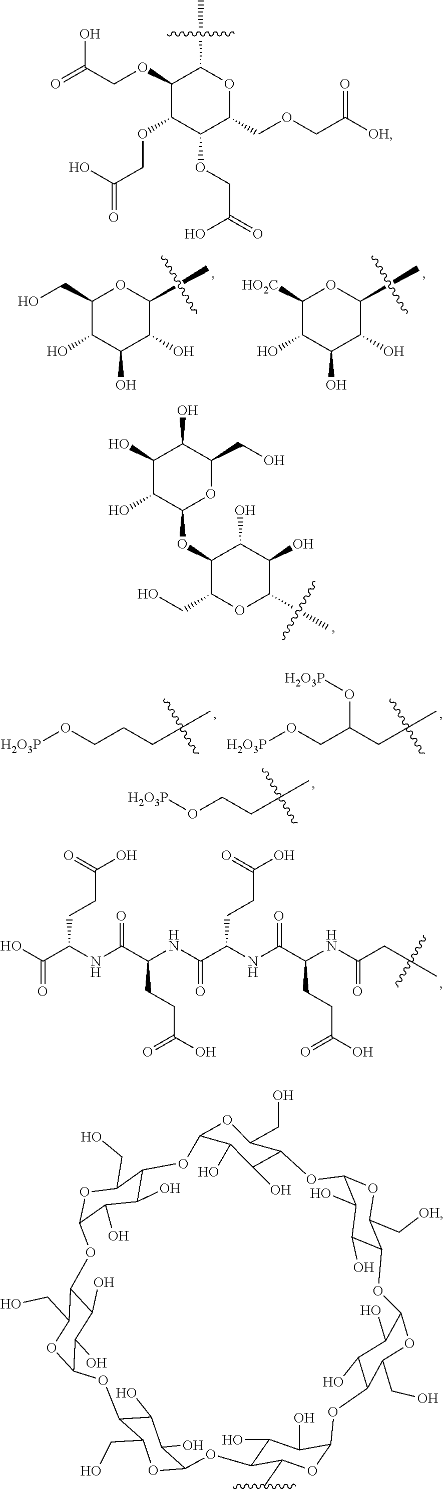 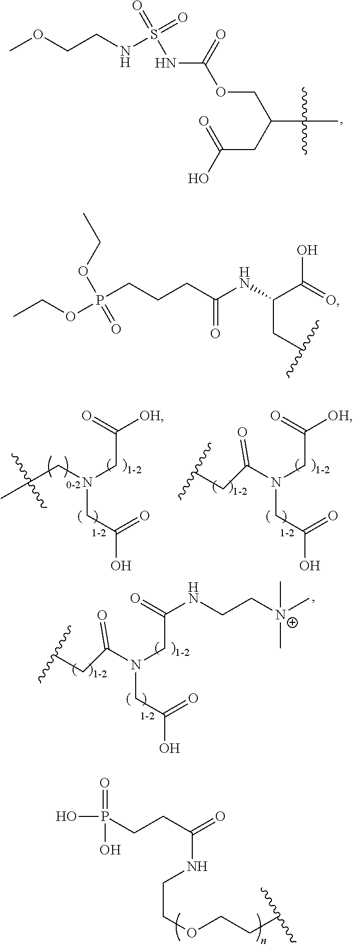
wherein n is an integer between 1 and 6, 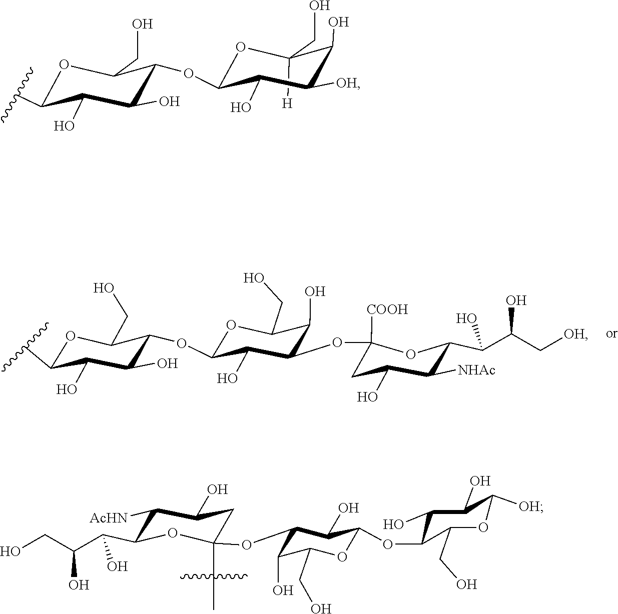
(iii) the hydrophilic group represented by R² or R³ each independently comprises:
(a) a polysarcosine with the following moiety: 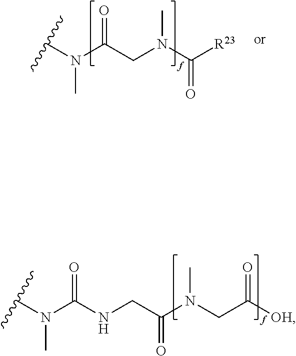
wherein
f is an integer between 3 and 25; and
R²³ is H, —CH₃ or —CH₂CH₂C(═O)OH; or
(b) a polyethylene glycol of formula: 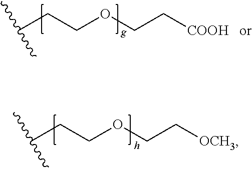
wherein g and h are independently an integer between 2 and 30;
(iv) the enzyme cleavage element represented by R² or R³ each independently comprises: 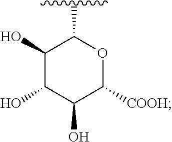 [SMILES: *[C@@H]1O[C@H](C(=O)O)[C@@H](O)[C@H](O)[C@H]1O]
or
(v) R² or R³, independently, is selected from a group consisting of 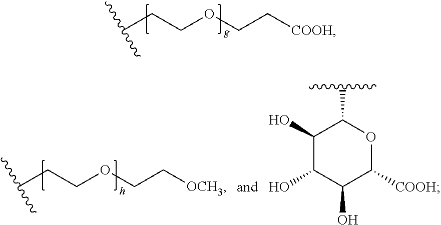
wherein
g and h are independently an integer between 20 and 30; preferably wherein
g is 23, 24, or 25; and
h is 23, 24, or 25.

65-69. (canceled)

70. The antibody-drug conjugate of claim 28, wherein:
i)the dual linker is represented by the following formula: 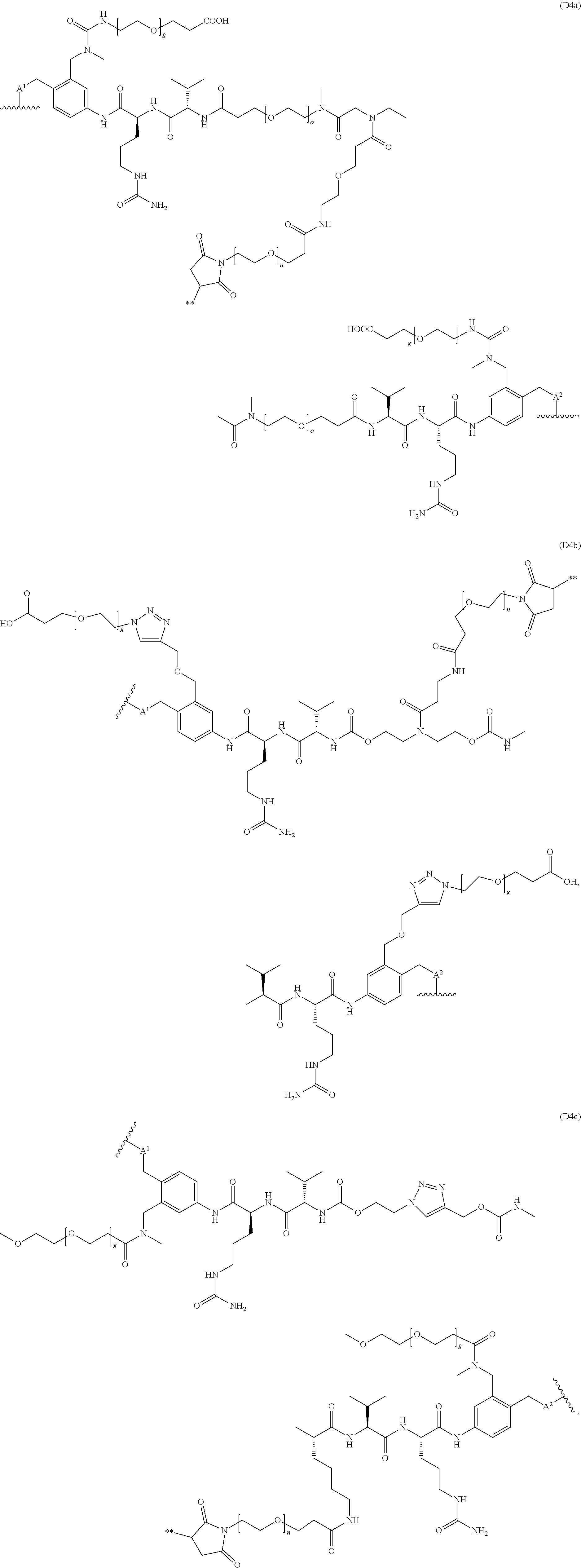 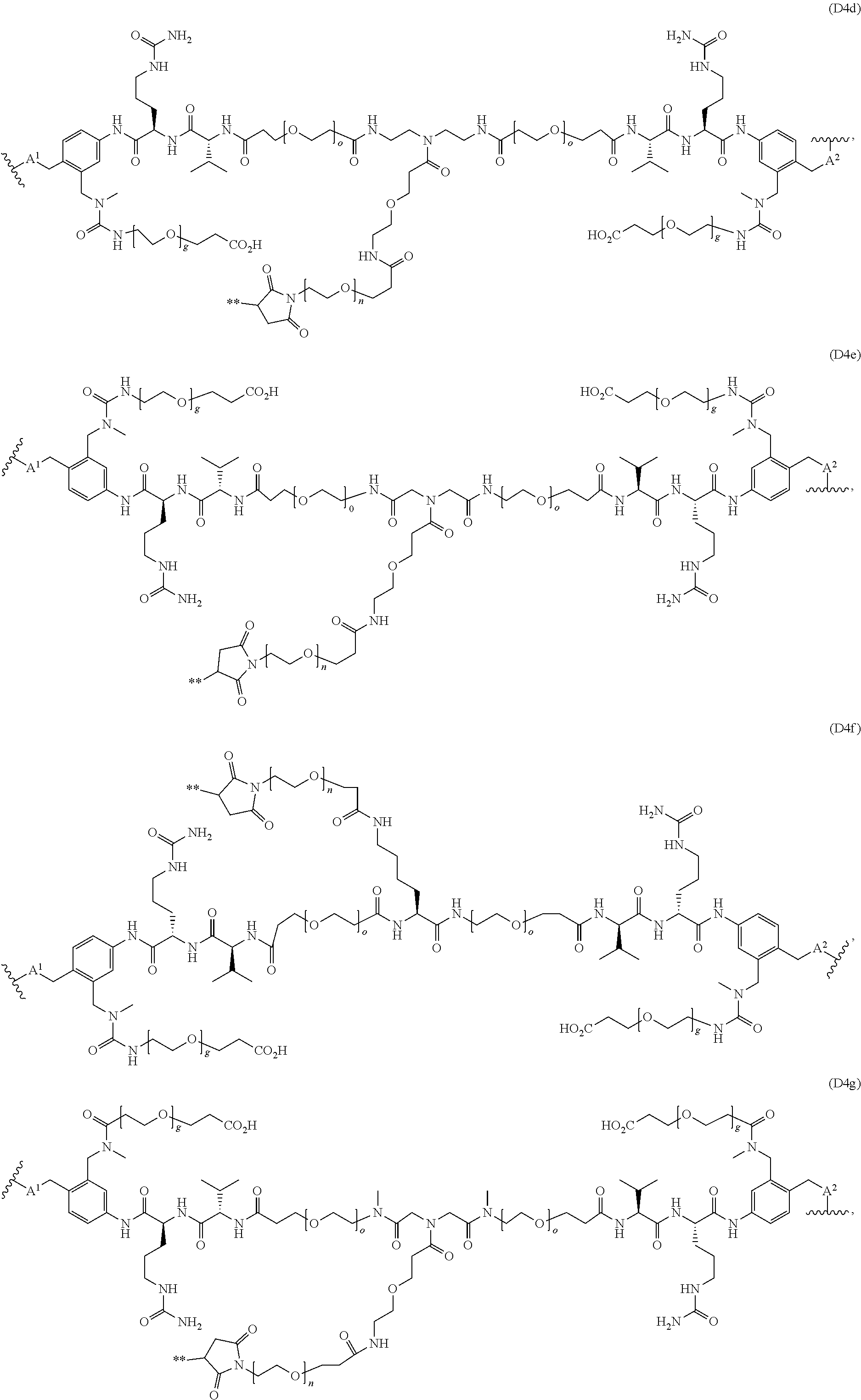 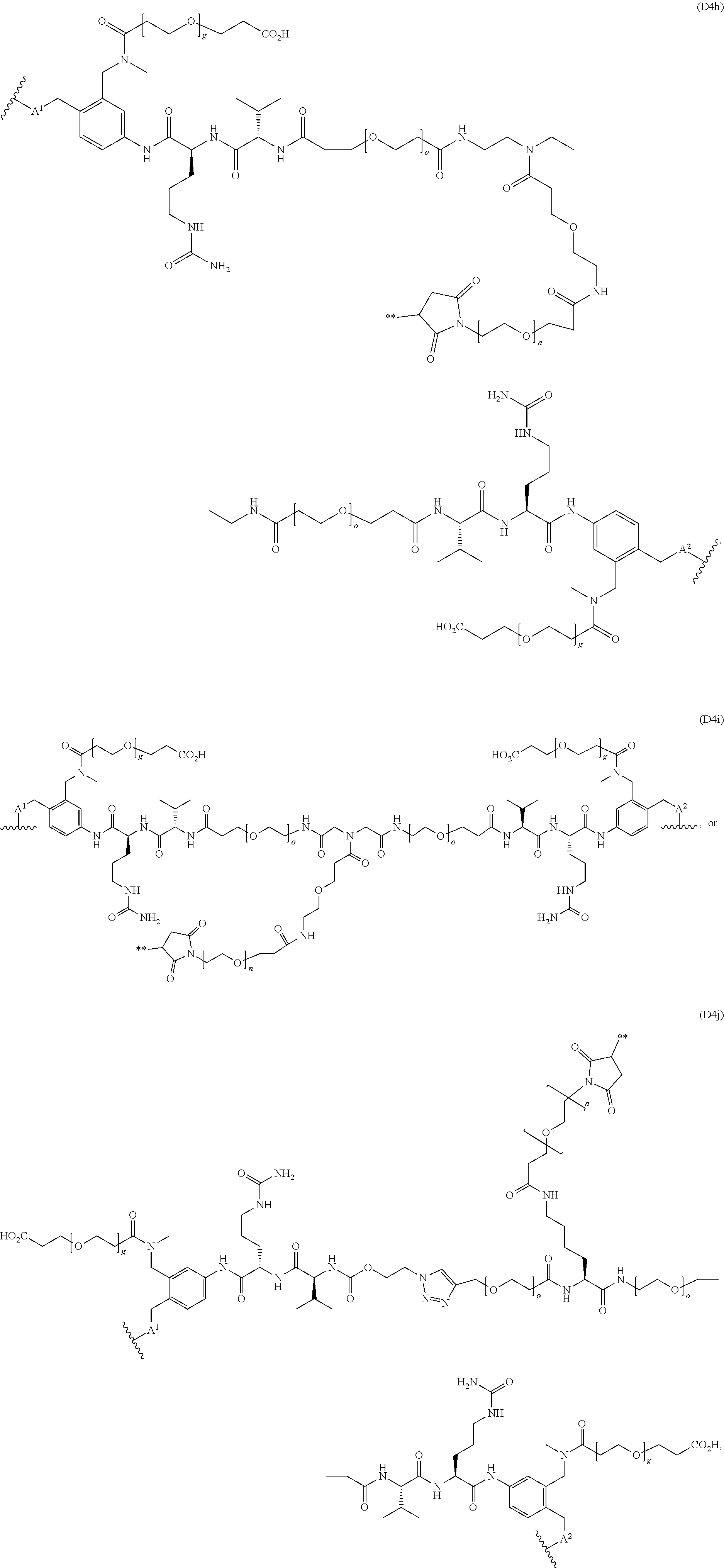
wherein:
A¹ and A² are each independently a bond, —O—C(═O)—*, or 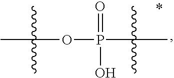 [SMILES: *OP(*)(=O)O]
preferably wherein A¹ and A² are each independently a bond or —O—C(═O)—* wherein * in A¹ and A² indicates the point of attachment to D¹ or D
g for each occurrence is independently an integer between 20 and 30 (e.g., 20, 21, 22, 23, 24, 25, 26, 27, 28, 29 or 30); o for each occurrence is independently an integer between 1 and 9 (e.g., between 2 and 5);
n is an integer between 1 and 12 (e.g., between 2 and 5); [symbol]
indicates the point of attachment to the Ab; and [symbol]
indicates the point of direct attachment to D¹ or D² or
(ii) the dual linker is represented by Formula (D5): 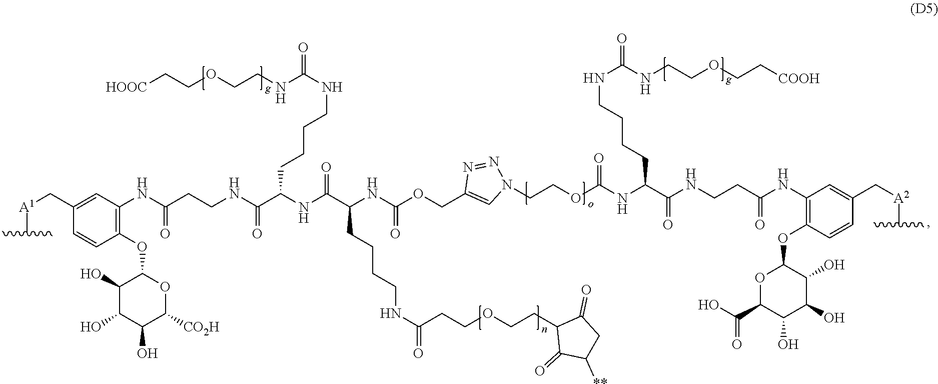
wherein:
A¹ and A² are each independent a bond, —O—C(═O)—*, or 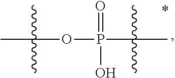 [SMILES: *OP(*)(=O)O]
preferably wherein A1 and A² are each independent a bond or —O—C(═O)—*, wherein * in A1 and A² indicates the point of attachment to D¹ or D² g for each occurrence is independently an integer between 20 and 30 (e.g., 20, 21, 22, 23, 24, 25, 26, 27, 28, 29 or 30);
o for each occurrence is independently an integer between 1 and 9 (e.g., between 1 and 3);
n is an integer between 1 and 12 (e.g., between 5 and 10); [symbol]
indicates the point of attachment to the Ab; and [symbol]
indicates the point of direct attachment to D¹ or D²; or
(iii) the dual linker is represented by the following formula: 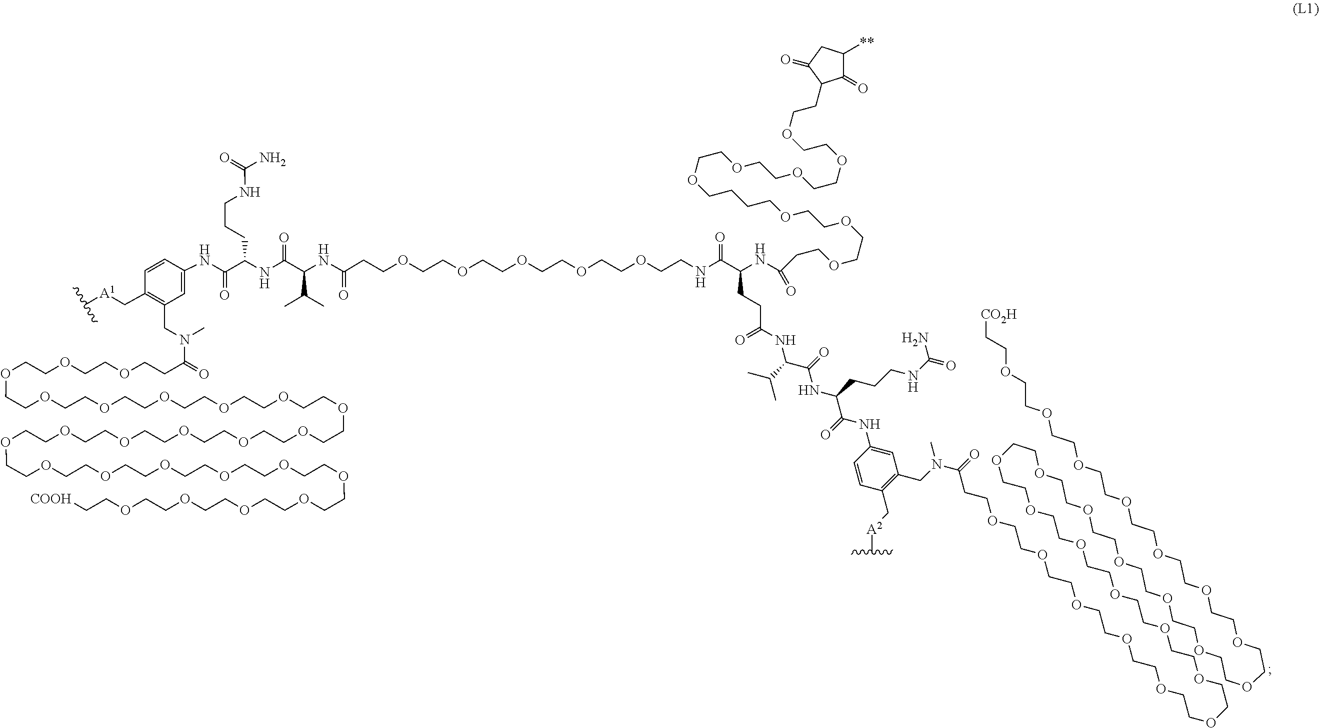 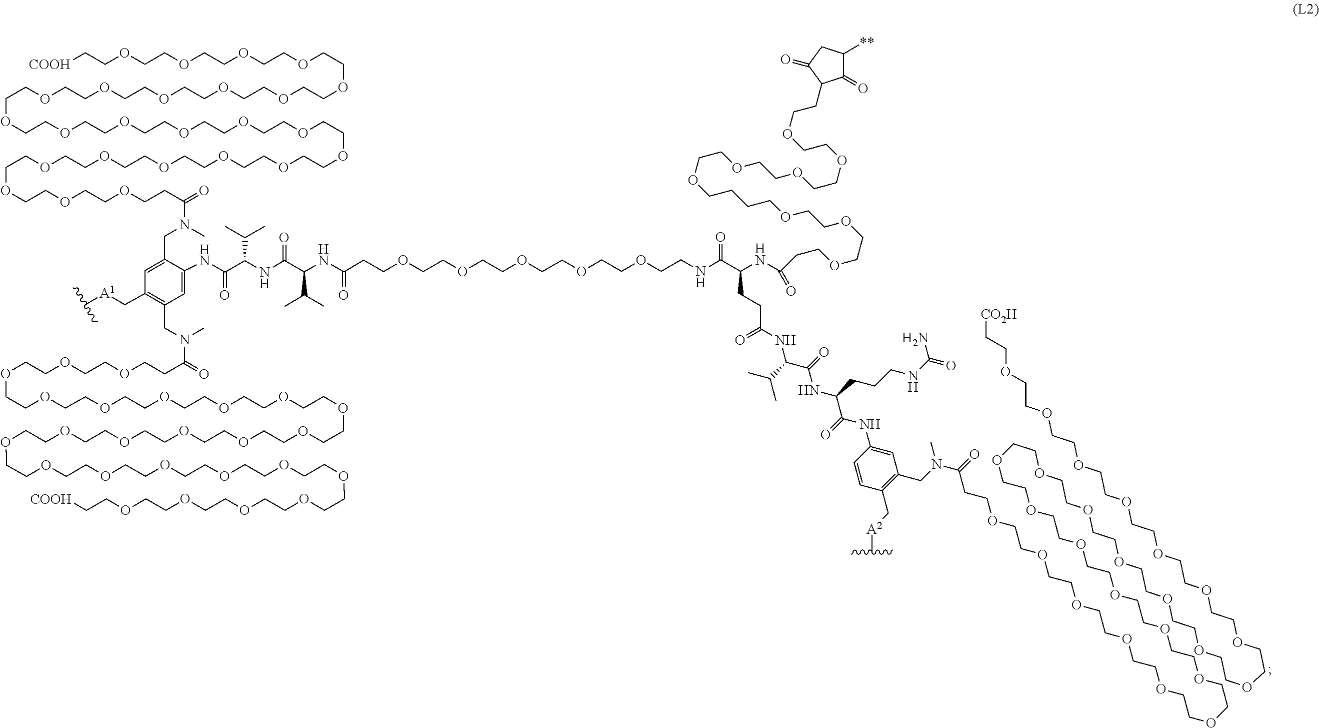 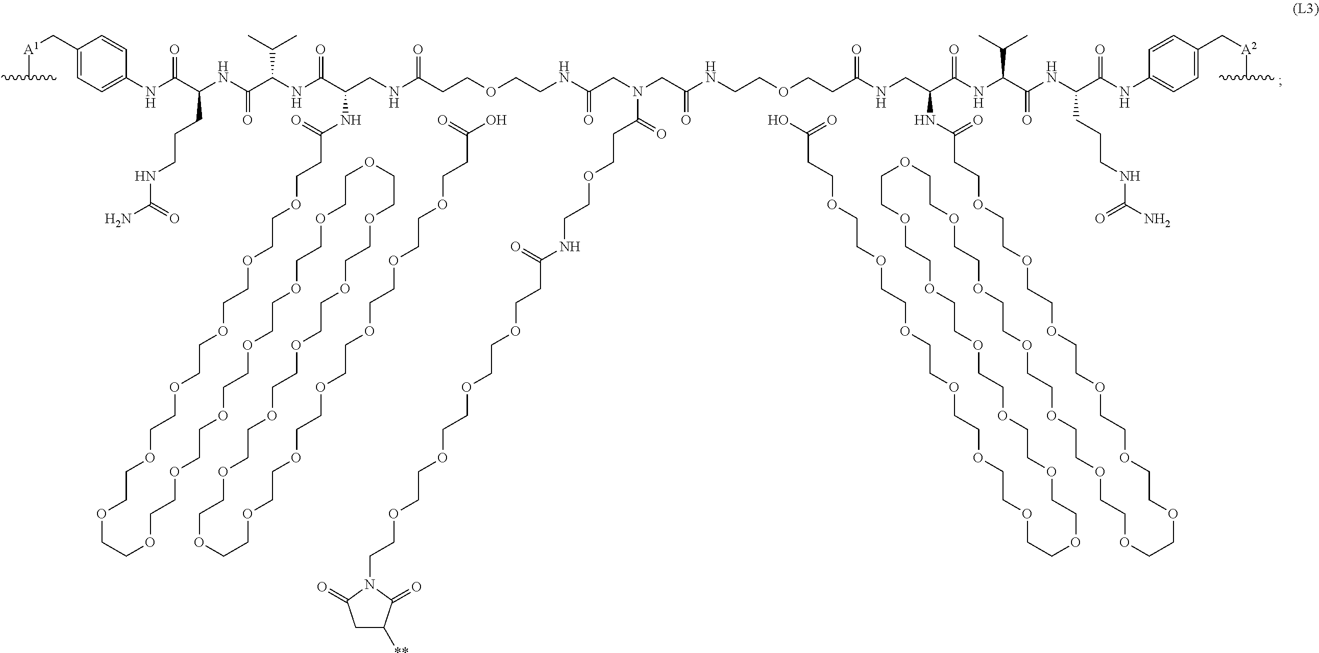 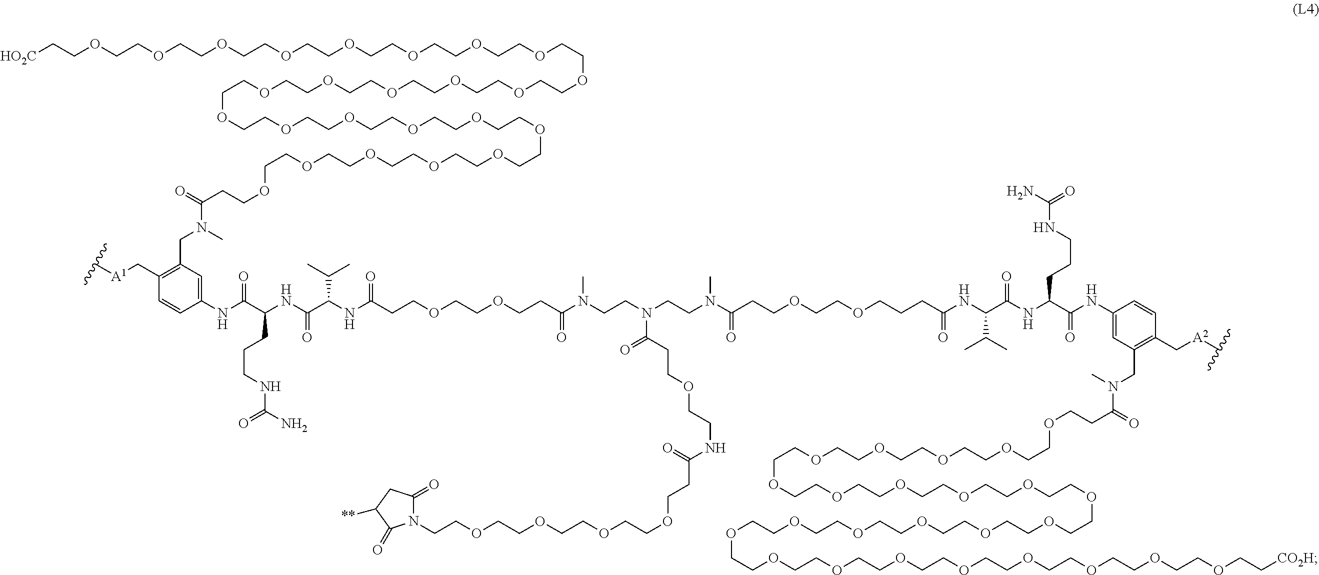 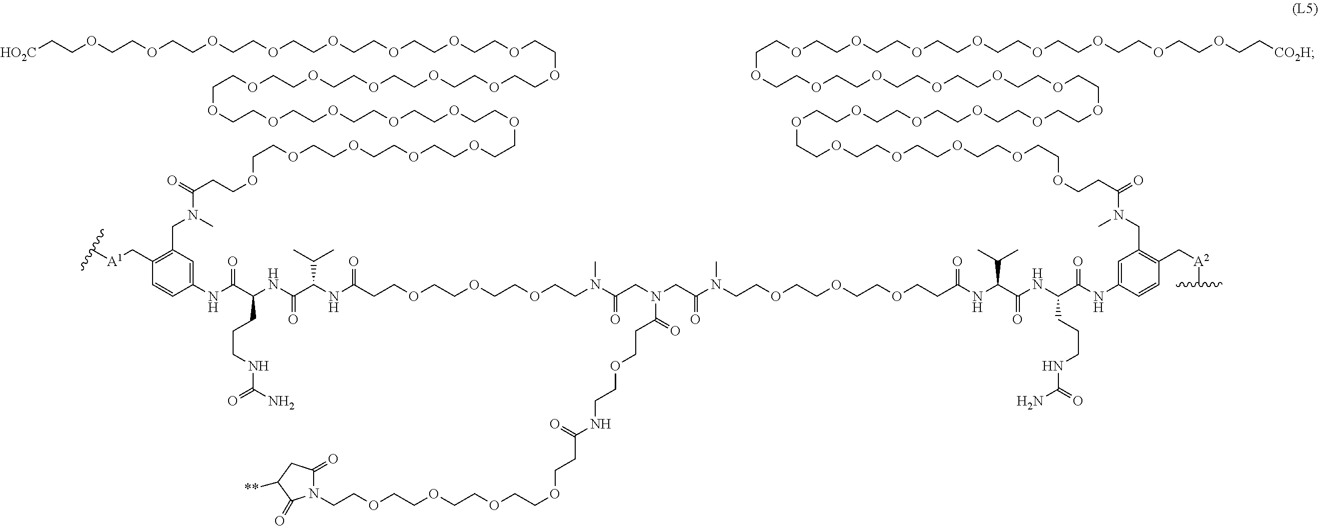 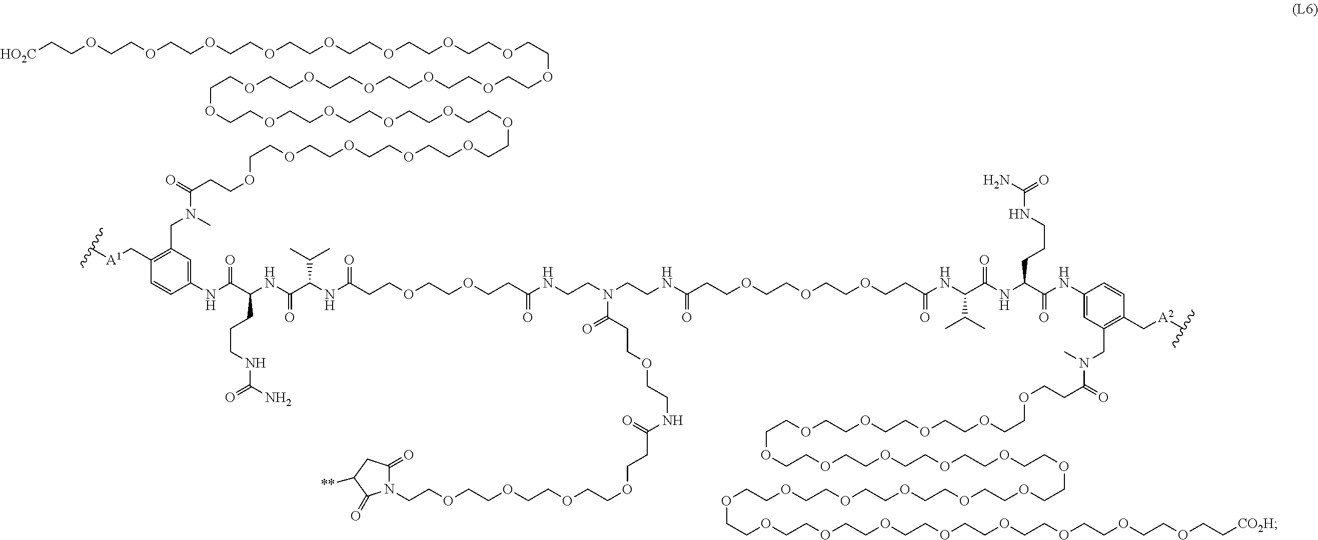 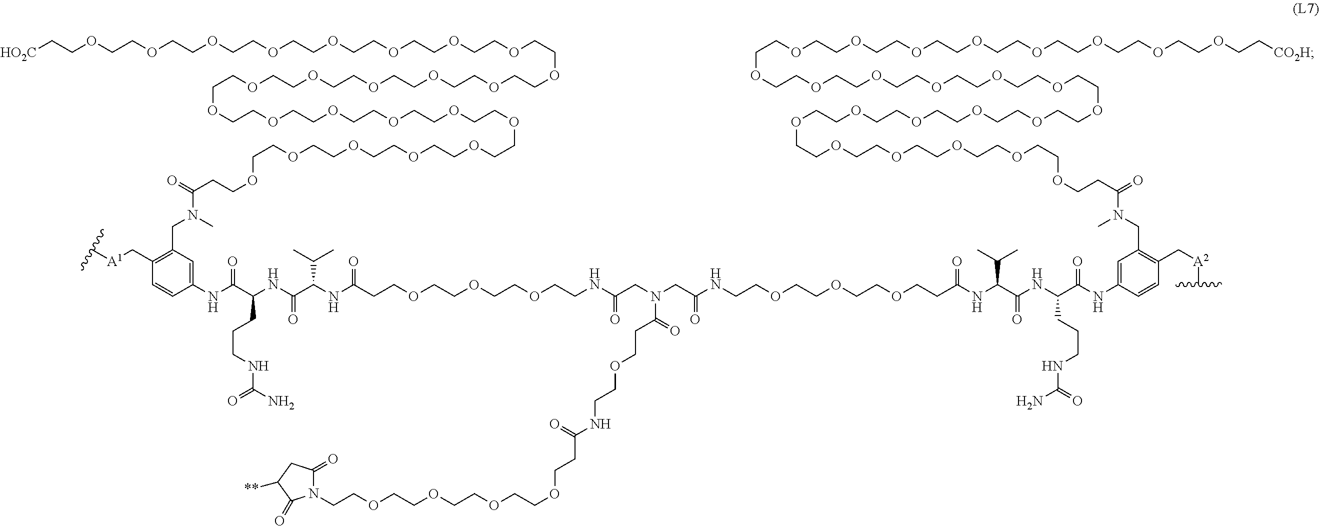 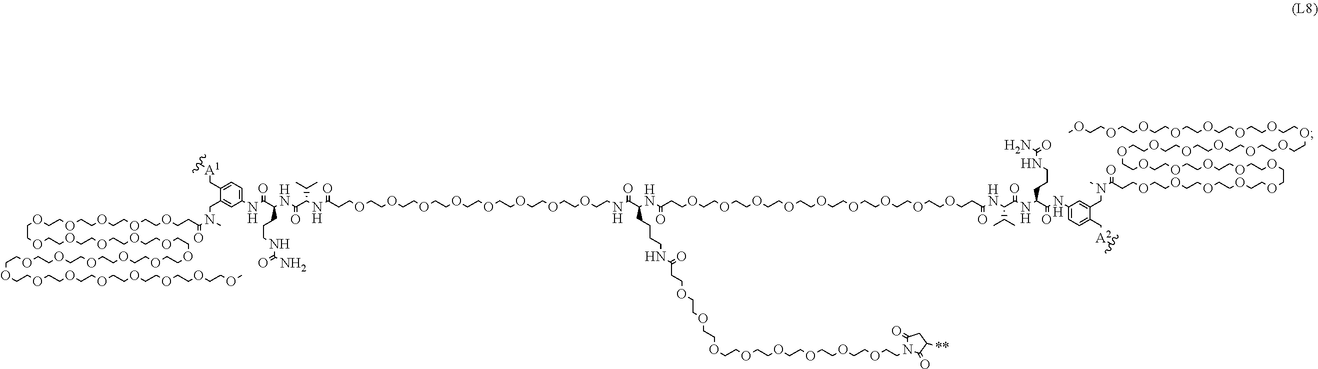 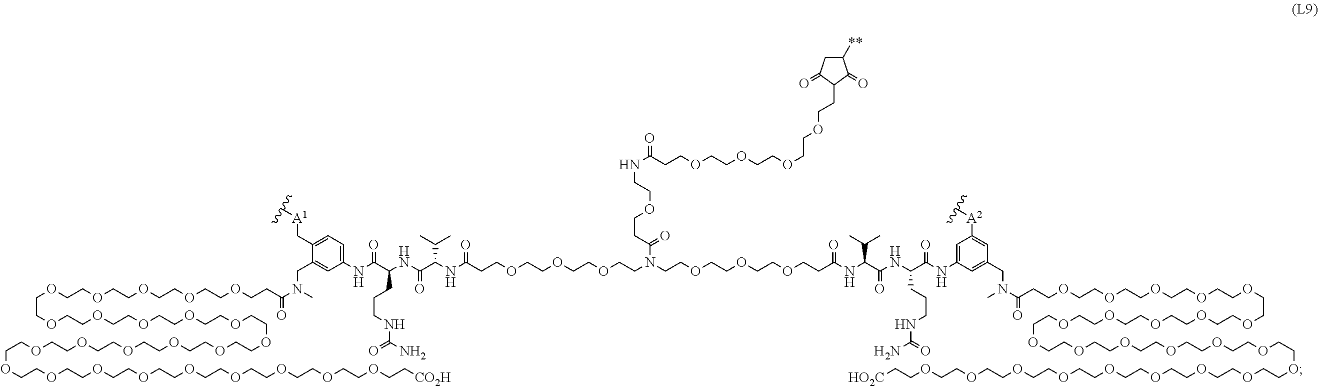 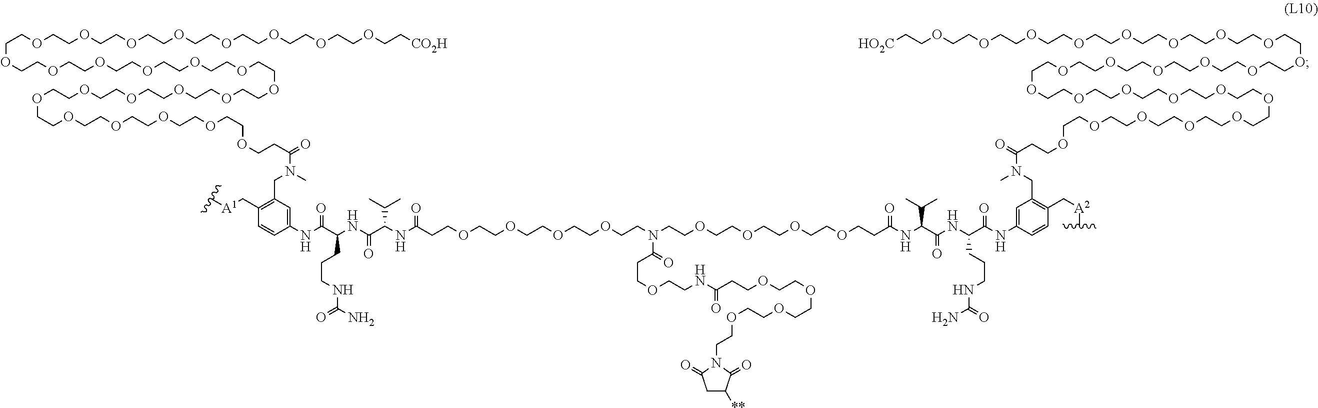 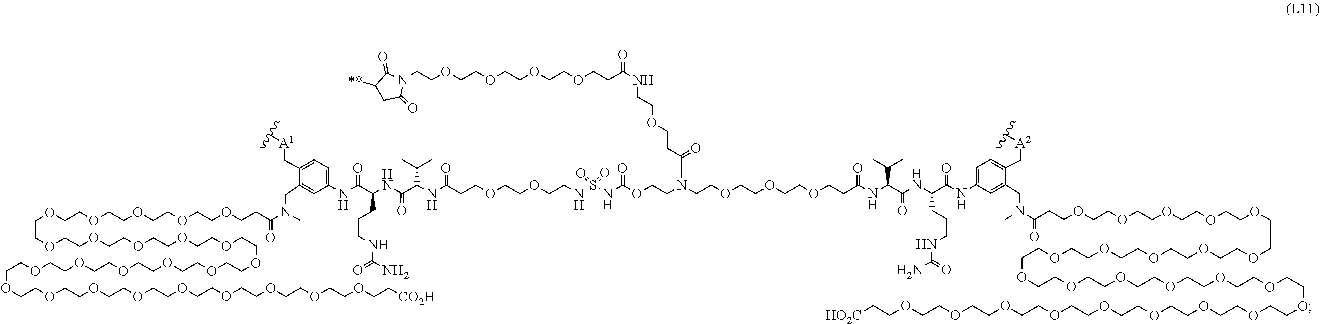 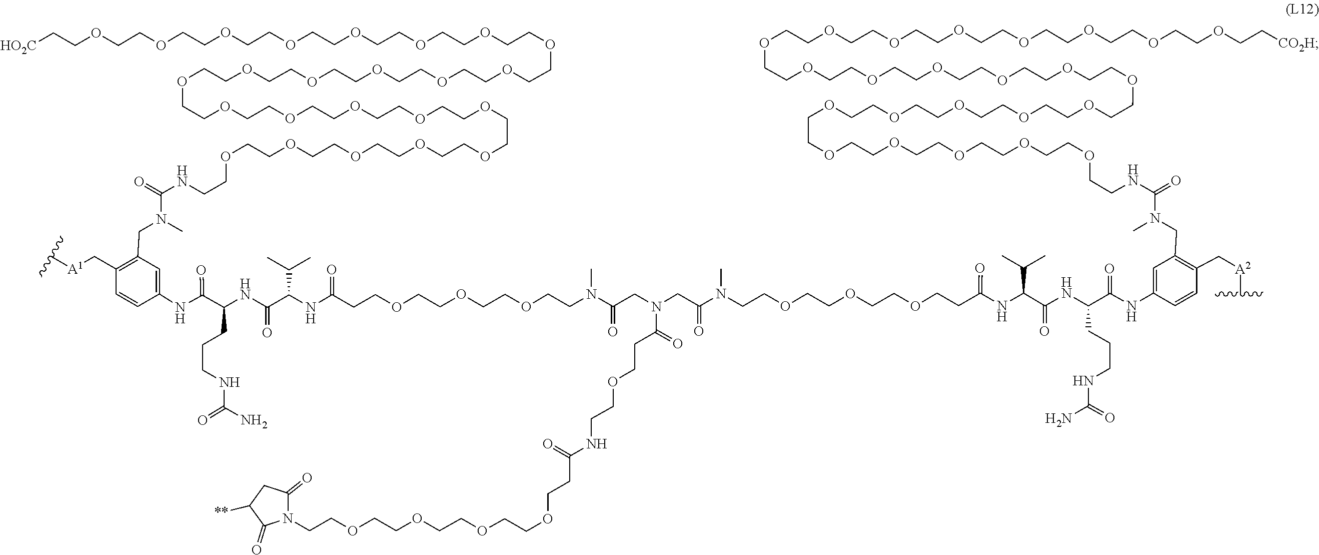 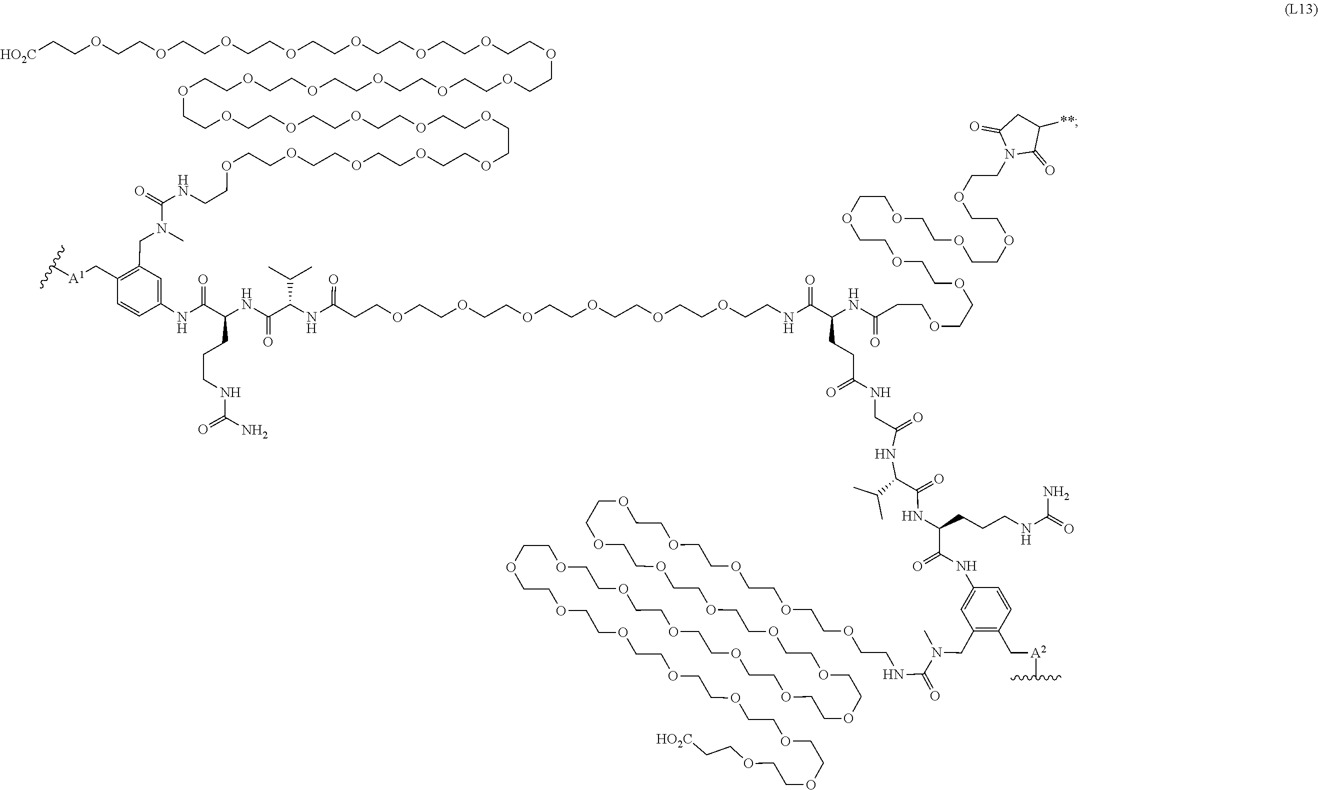 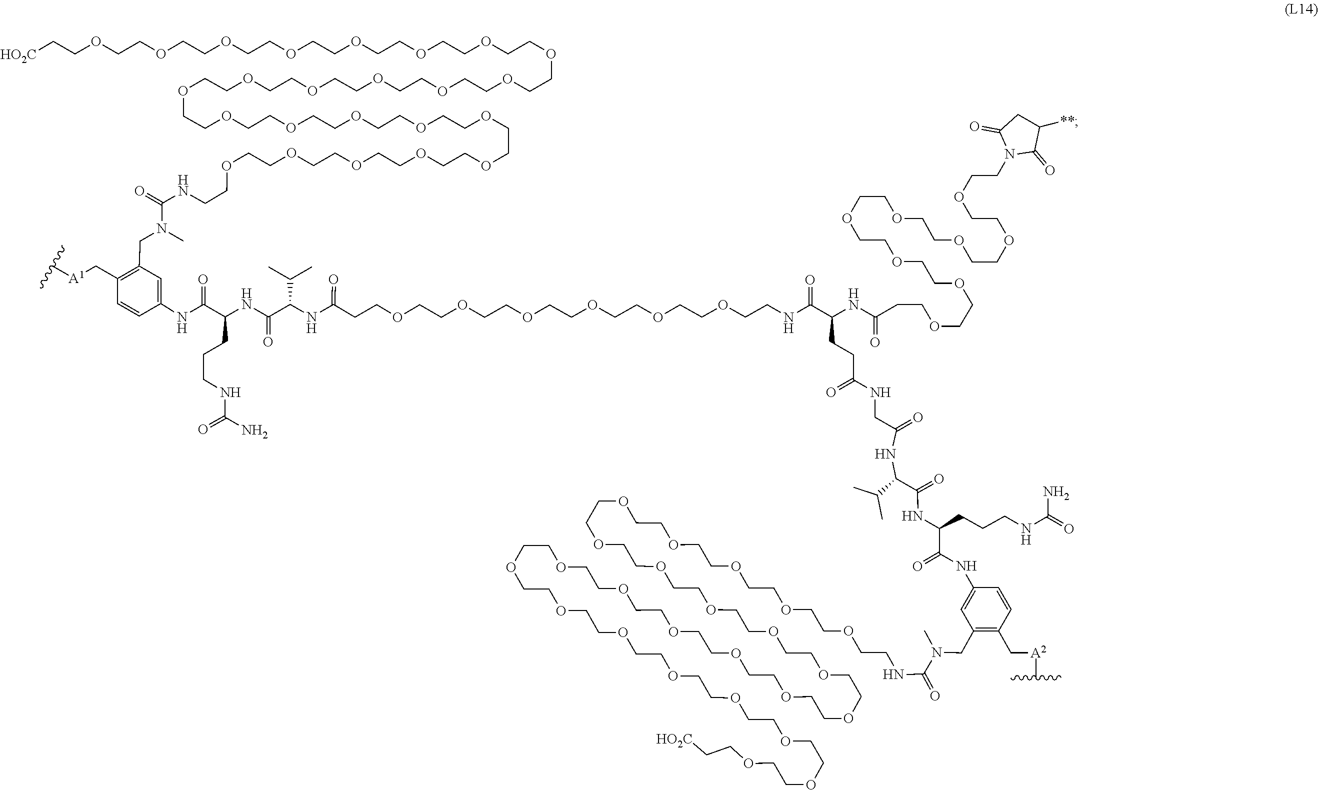 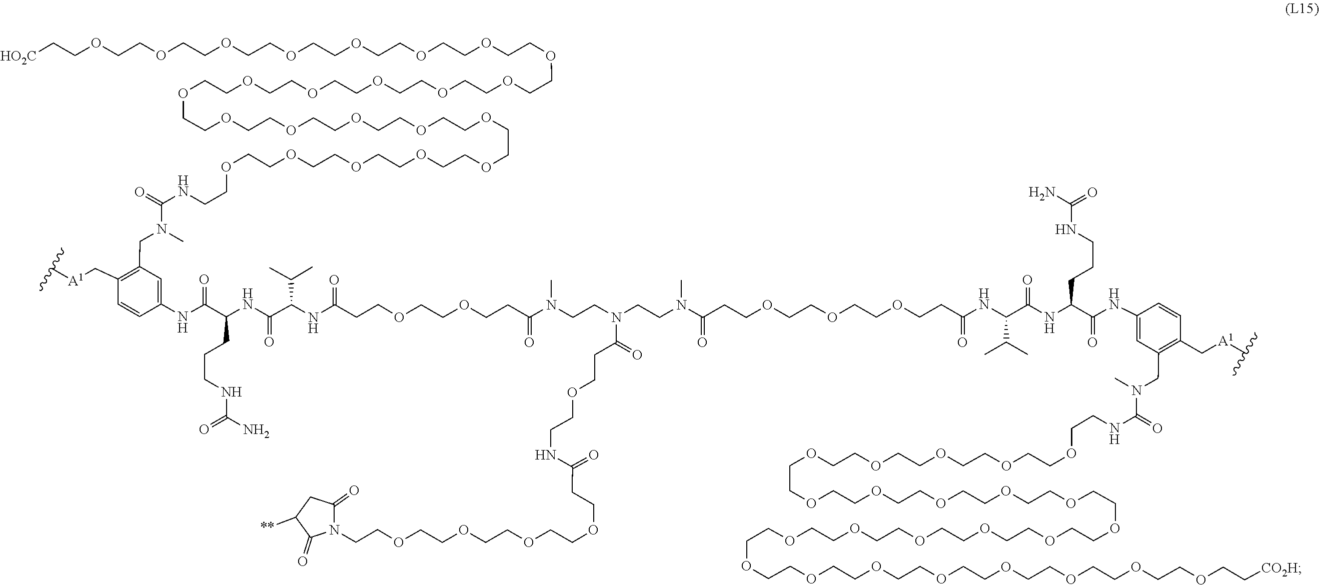 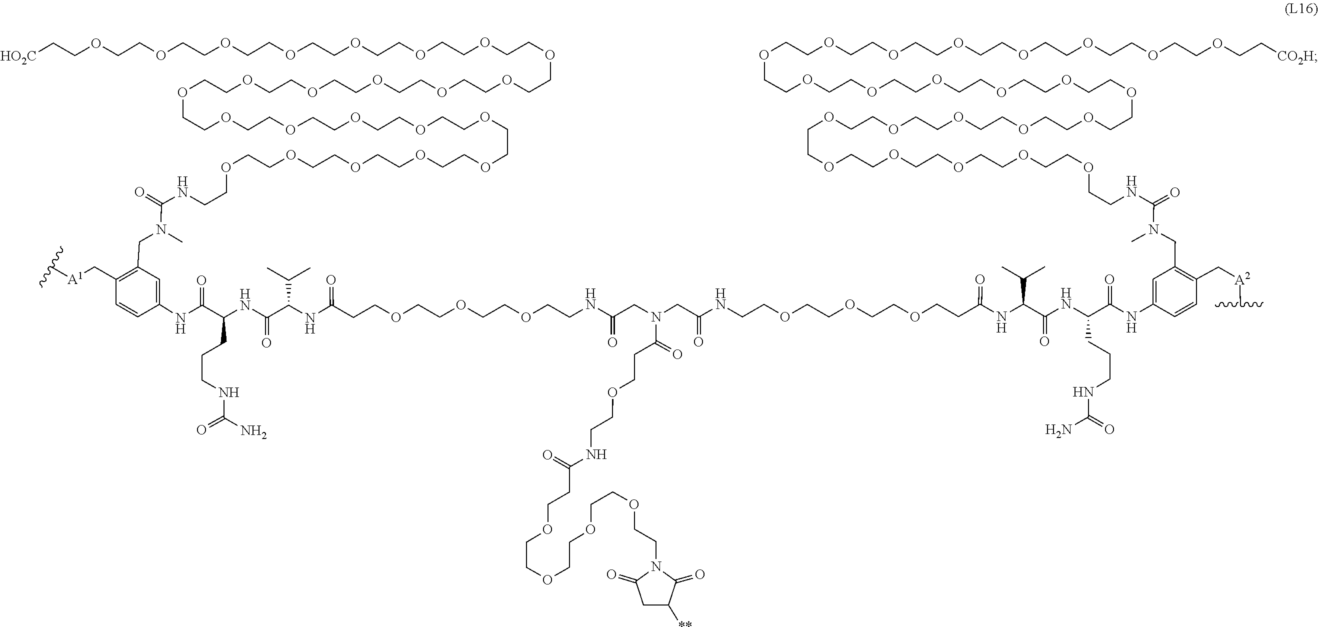 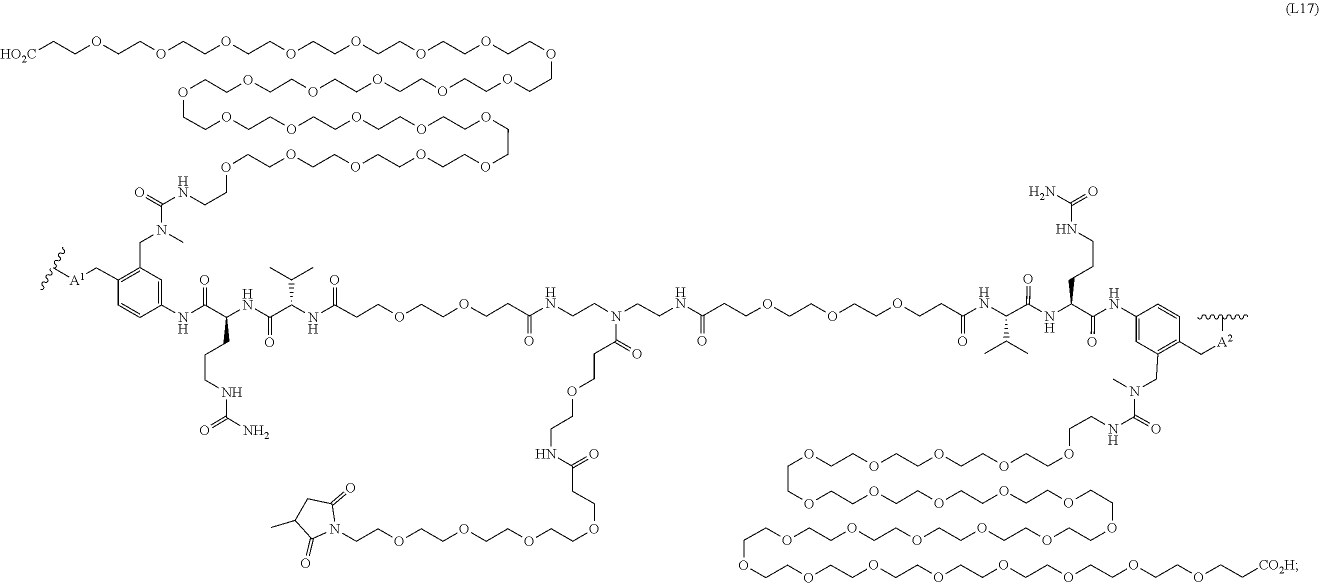 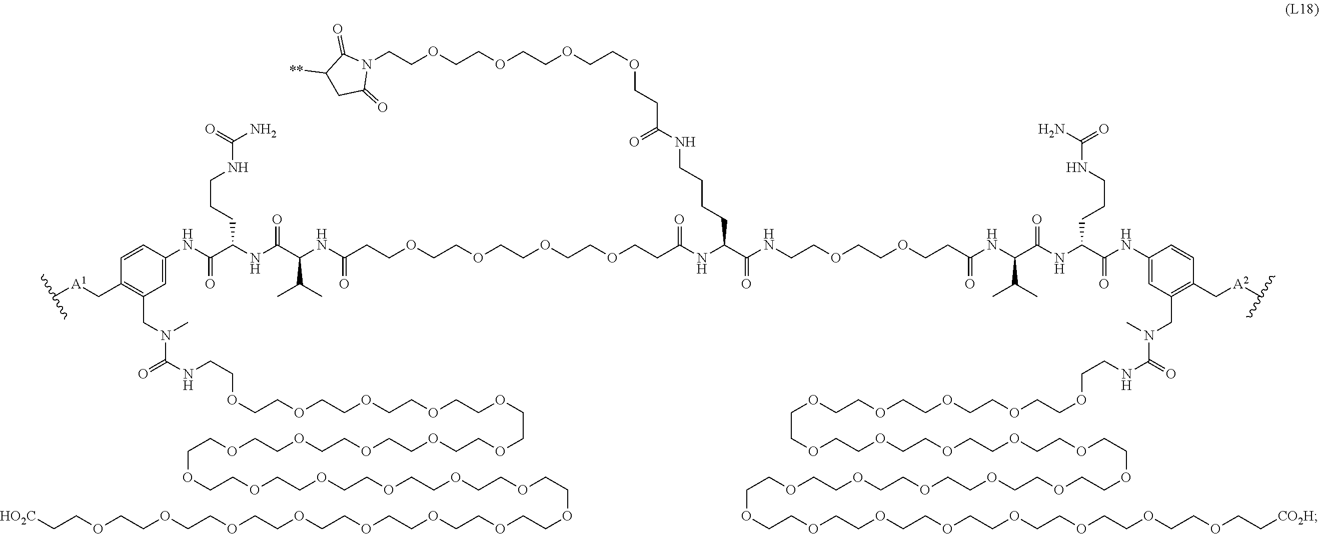 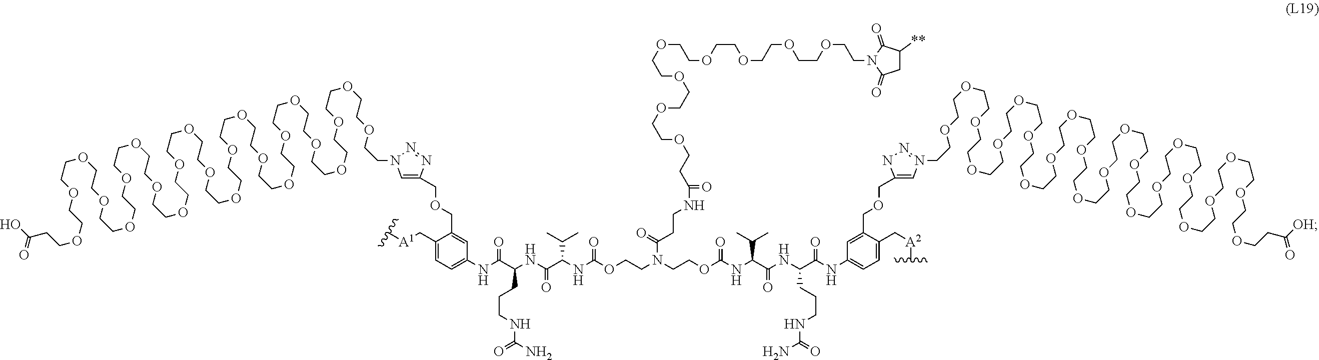 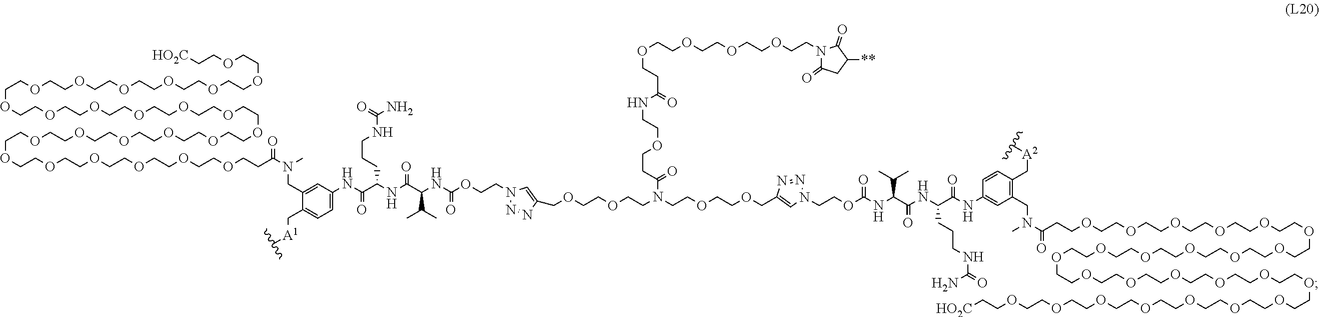 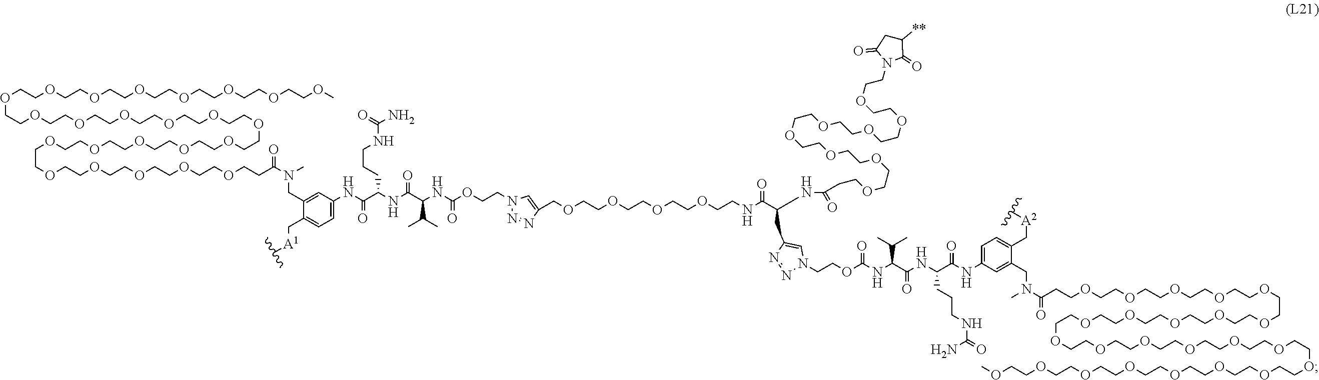 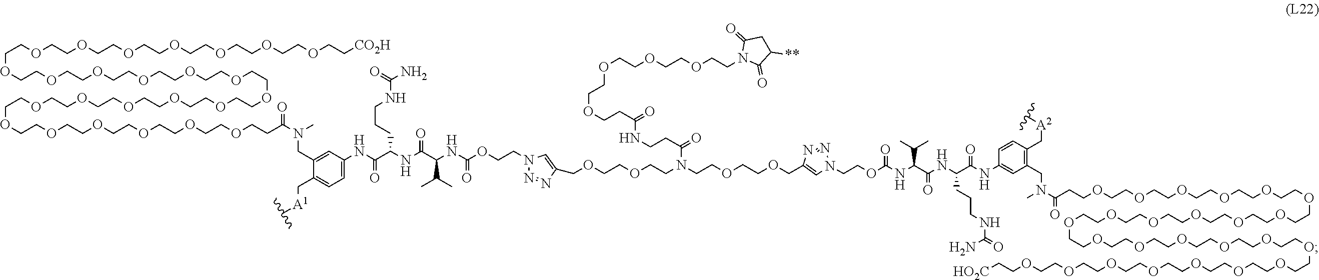 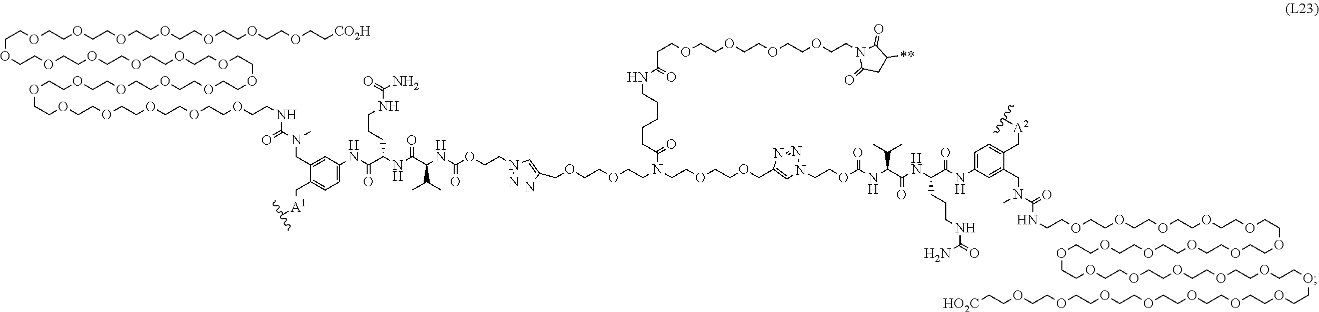 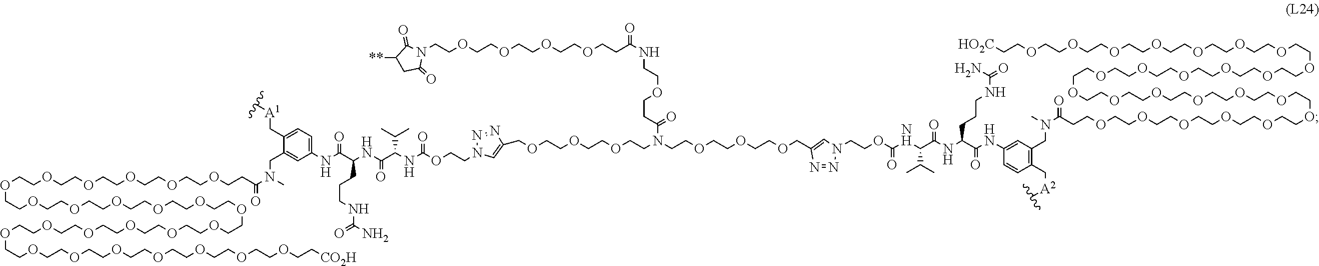 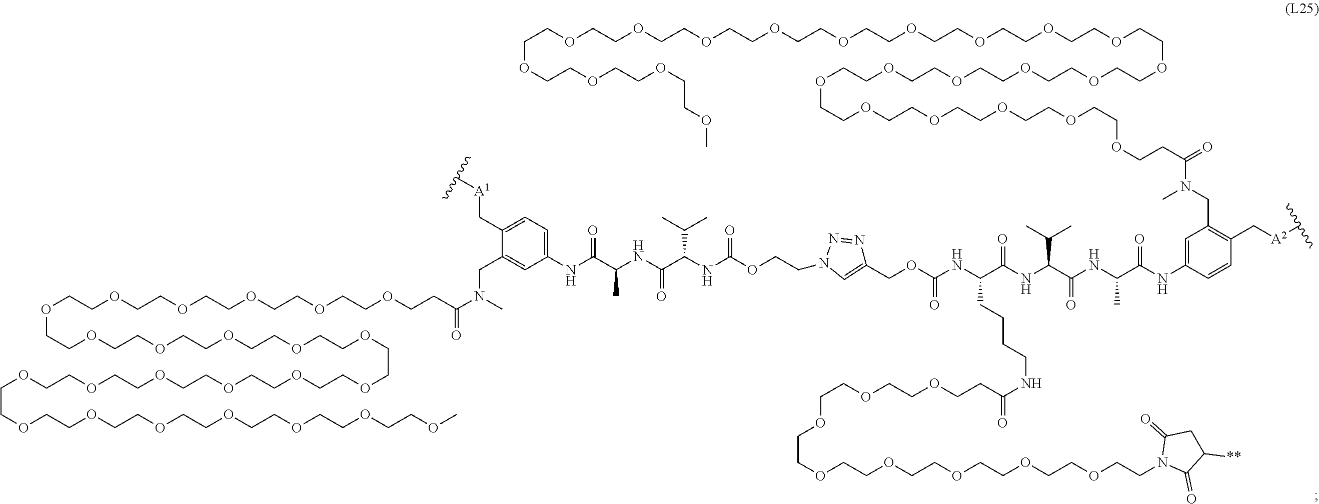 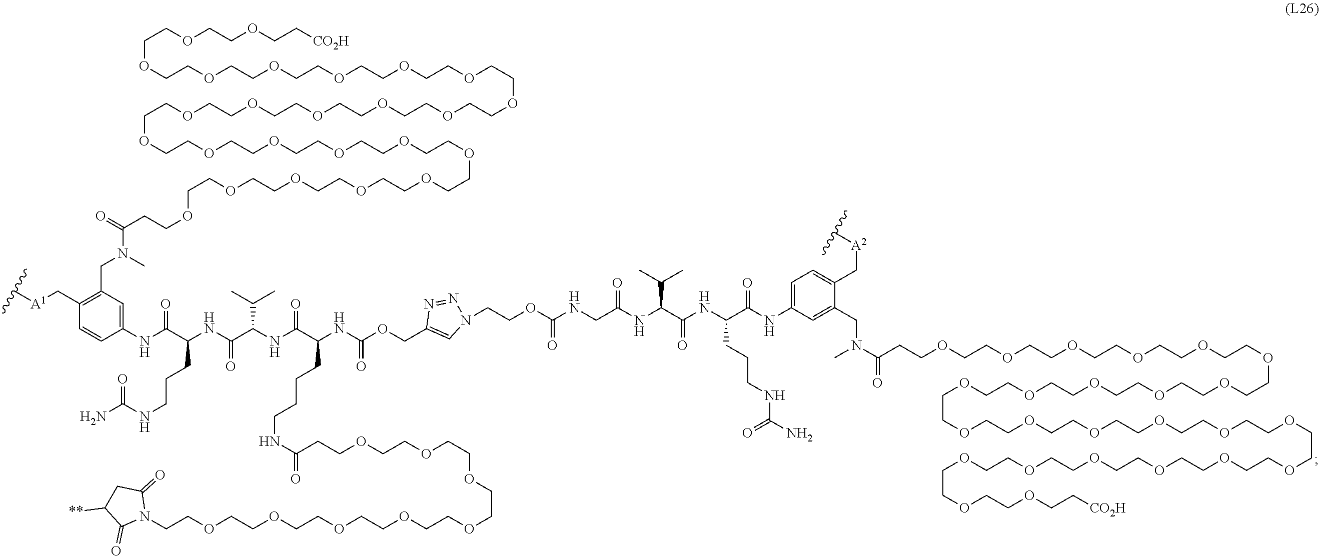 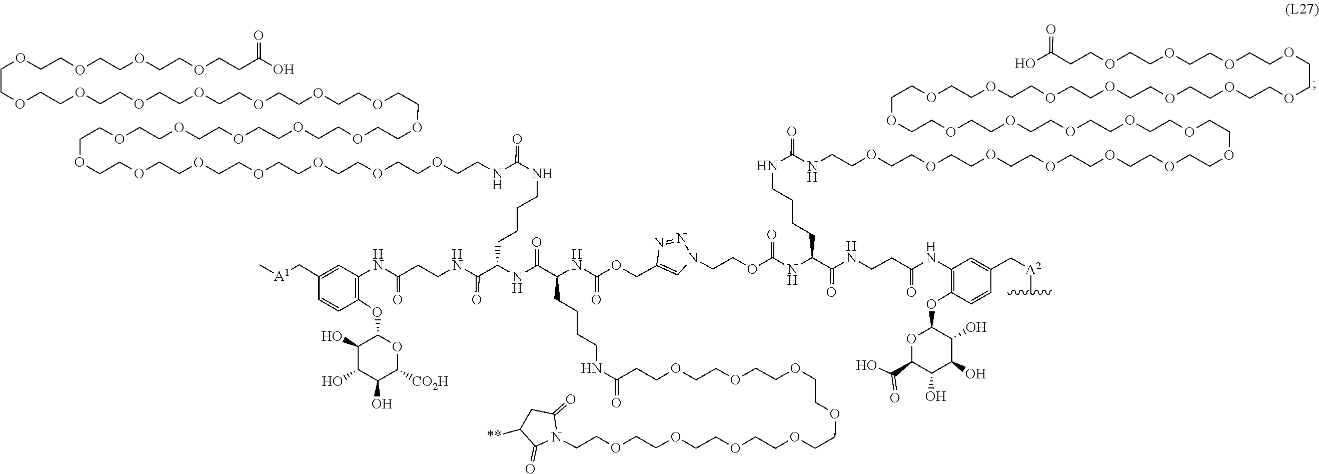 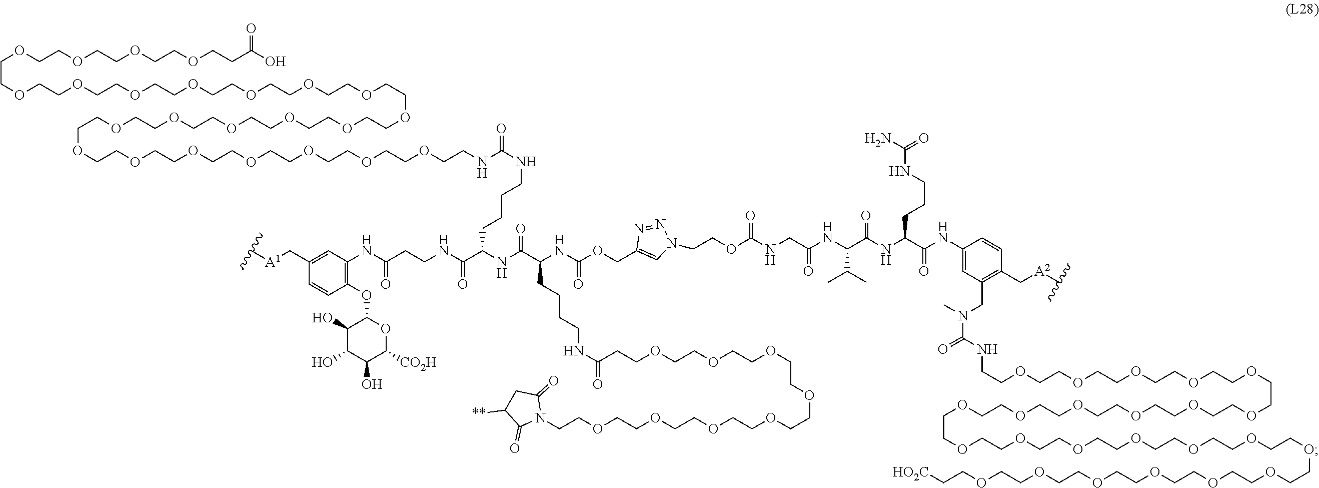 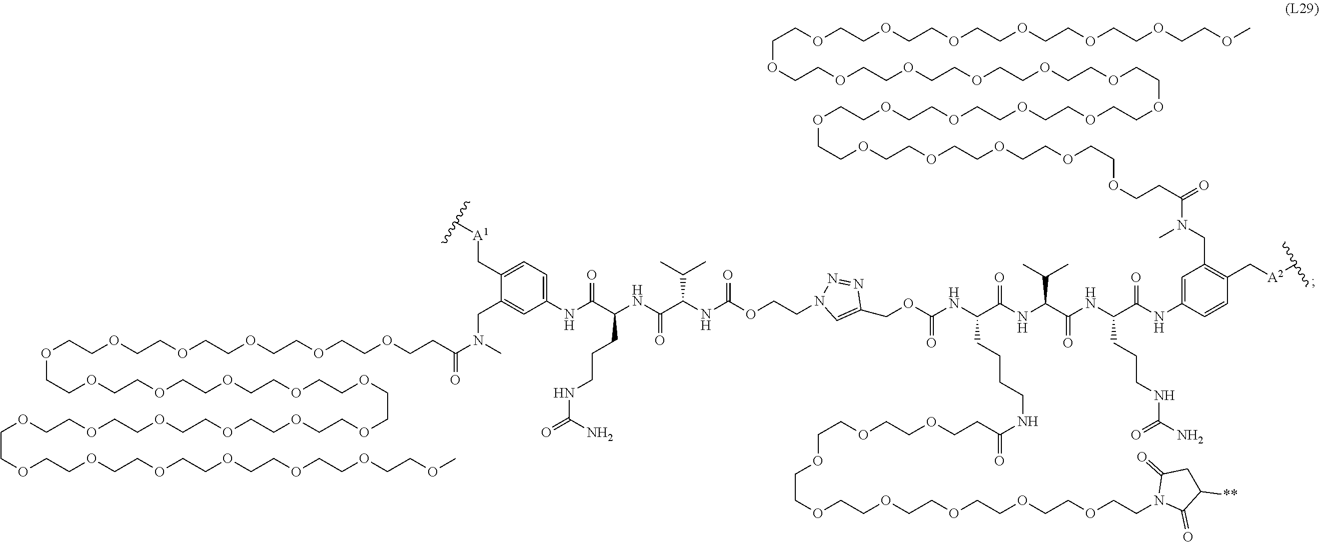 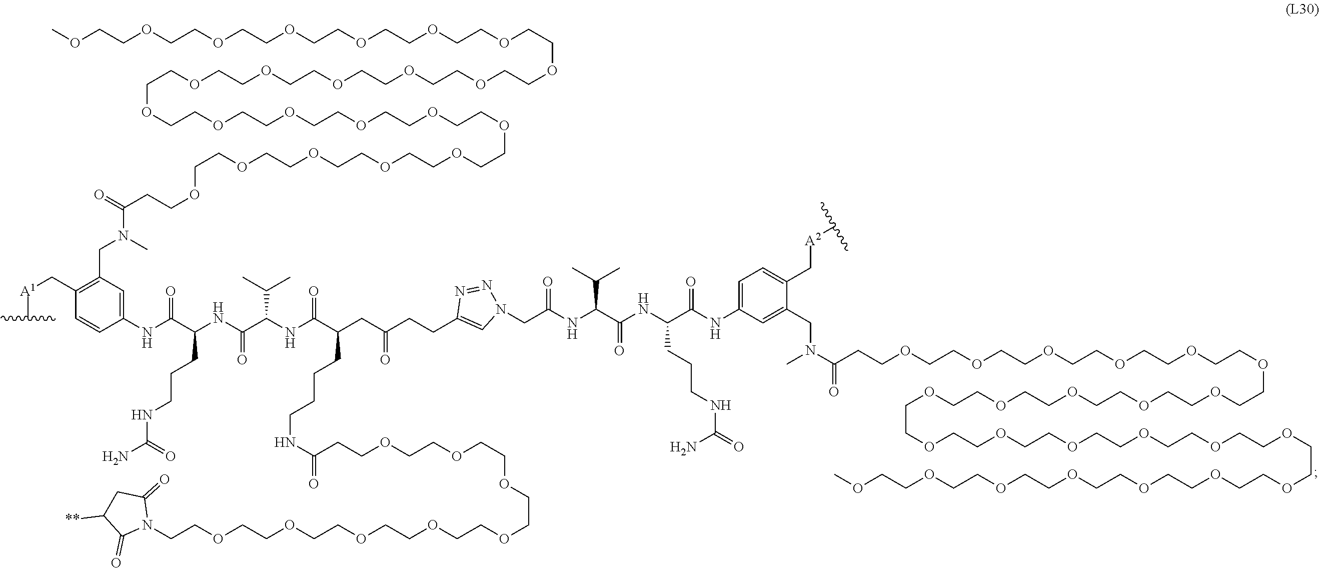 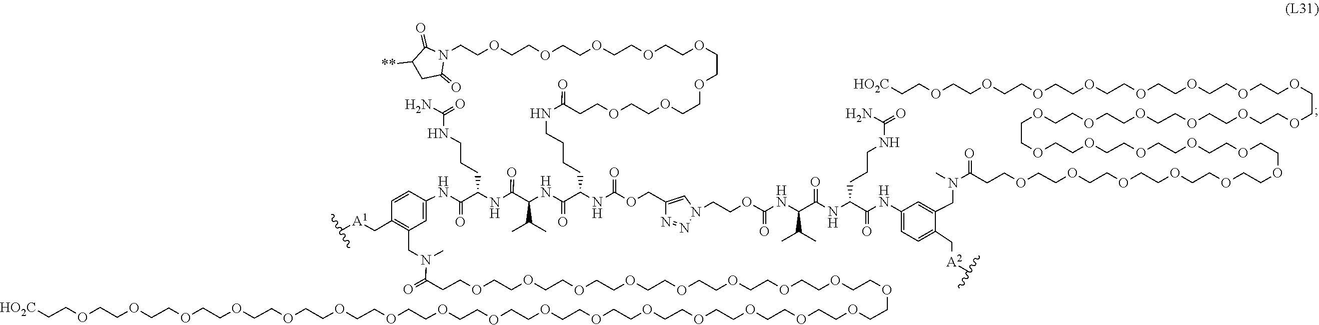 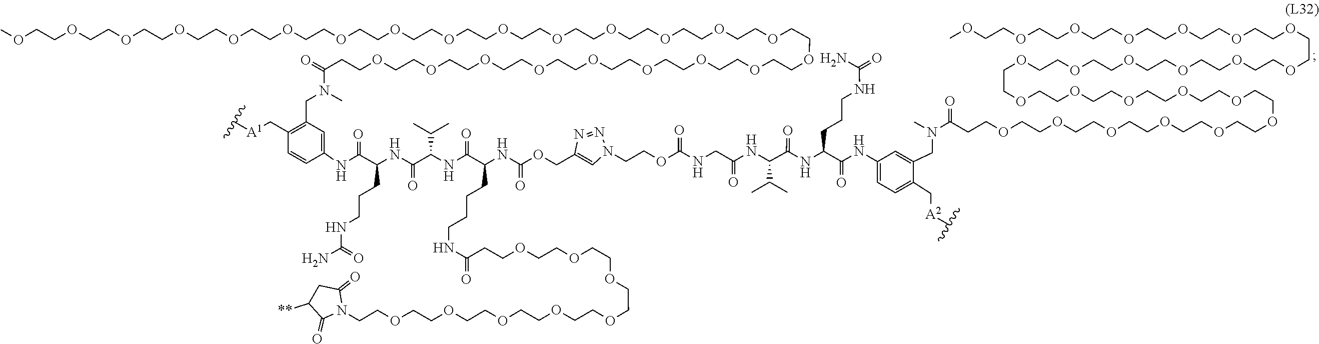 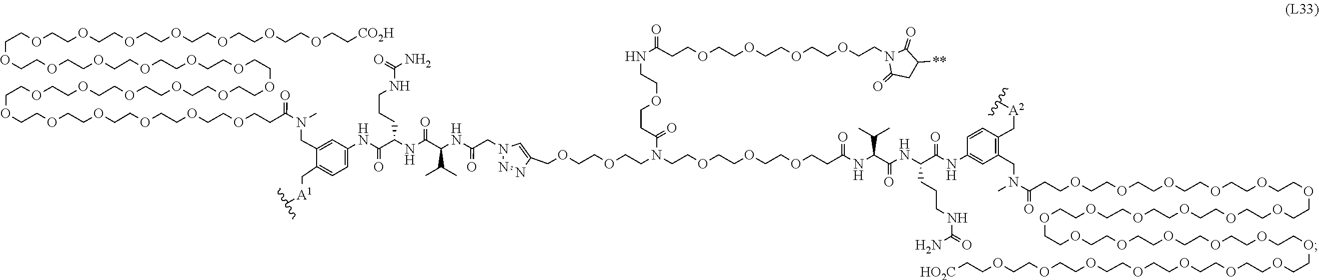 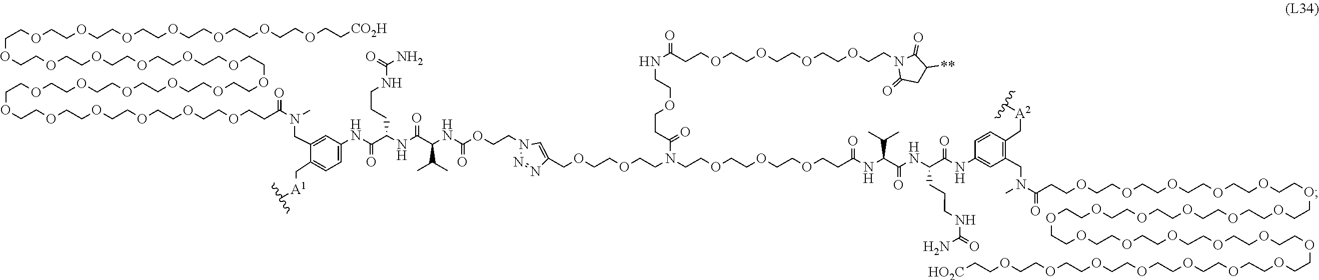 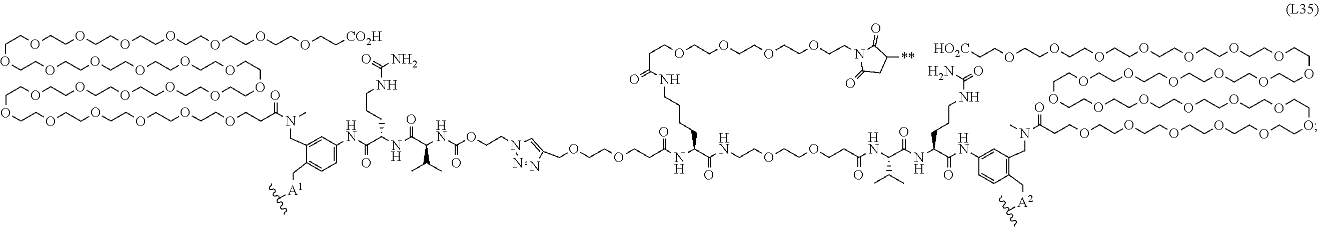 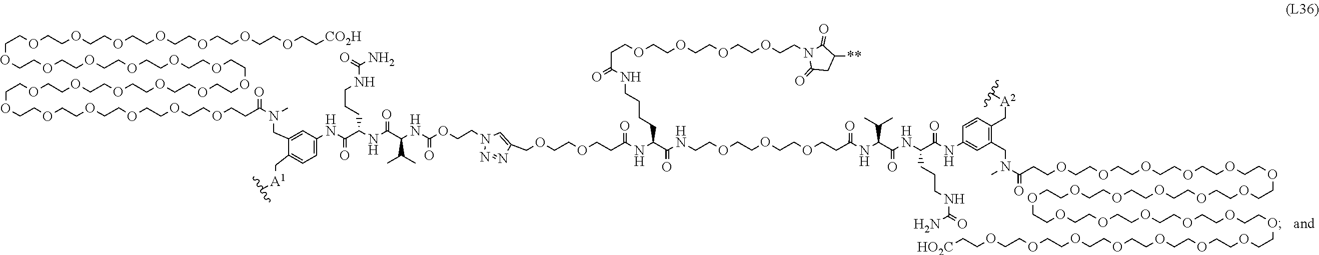 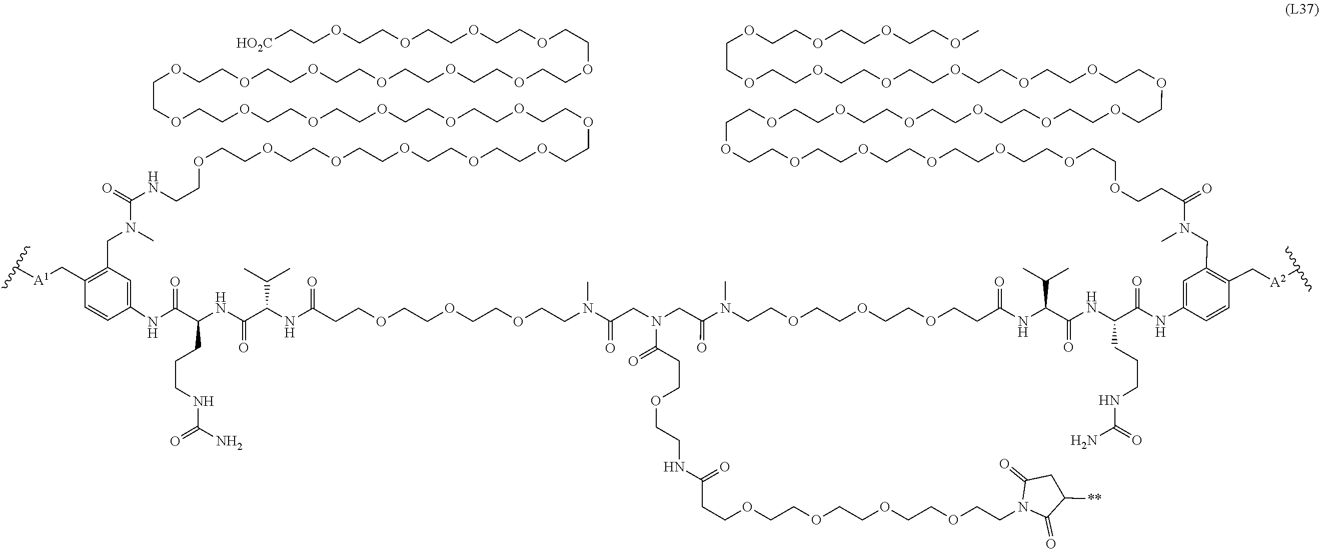
wherein each A¹ or A² independently is a bond, —OC(═O)—*, or 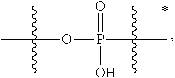 [SMILES: *OP(*)(=O)O]
preferably wherein A¹ and A² are each independent a bond or —O—C(═O)—*, wherein * indicates the point of attachment to the antineoplastic payload; [symbol] indicates the point of attachment to the Ab; and [symbol] indicates the point of direct attachment to the antineoplastic payload, wherein at least one antineoplastic payload is a BH3 mimetic; preferably wherein D¹ and D² are each independently a BH3 mimetic.

71-77. (canceled)

78. The antibody-drug conjugate of claim 19, wherein;
(i) one of D¹ and D² is a BH3 mimetic selected from a Mcl-1 inhibitor, a Bcl-2 inhibitor, and a Bcl-xL inhibitor, and the other is an antineoplastic non-BH3 mimetic selected from topoisomerase 1 inhibitor or an anti-mitotic drug;
(ii) D¹ and/or D² are each independently selected from a Mcl-1 inhibitor, a Bcl-2 inhibitor, and a Bcl-xL inhibitor;
(iii) both D¹ and D² are (a) a Mcl-1 inhibitor; (b) a Bcl-2 inhibitor; or (c) a Bcl-xL inhibitor;
(iv) D¹ and D² are the same;
(v) D¹ and D² are different;
(vi) one of D¹ and D² is a Mcl-1 inhibitor and the other is a Bcl-2 inhibitor;
(vii) one of D¹ and D² is a Mcl-1 inhibitor and the other is a Bcl-xL inhibitor;
(viii) one of D¹ and D² is a Bcl-2 inhibitor and the other is a Bcl-xL inhibitor;
(ix) D¹ is a Mcl-1 inhibitor and D² is a Mcl-1 inhibitor;
(x) D¹ is a Mcl-1 inhibitor and D² is a Bcl-2 inhibitor;
(xi) D¹ is a Bcl-xL inhibitor and D² is a Bcl-xL inhibitor:
(xii) D¹ is a Bcl-xL inhibitor and D² is a Bcl-2 inhibitor;
(xiii) D¹ is a Bcl-2 inhibitor and D² is a Mcl-1 inhibitor;
(xiv) D¹ is a Mcl-1 inhibitor and D² is a Bcl-xL inhibitor;
(xv) D¹ is a BH3 mimetic and D² is an antineoplastic non-BH3 mimetic;
(xvi) D¹ is selected from a Mcl-1 inhibitor, a Bcl-2 inhibitor, and a Bcl-xL inhibitor, and D² is a topoisomerase 1 inhibitor or an anti-mitotic drug;
(xvii) D¹ is a Bcl-xL inhibitor and D² is a topoisomerase 1 inhibitor; or
(xviii) D¹ is a Bcl-xL inhibitor and D² is an anti-mitotic drug.

79-88. (canceled)

89. The antibody-drug conjugate of claim 70, or pharmaceutically acceptable salt thereof, wherein the Mcl-1 inhibitor is represented by:
(i) Formula (I): 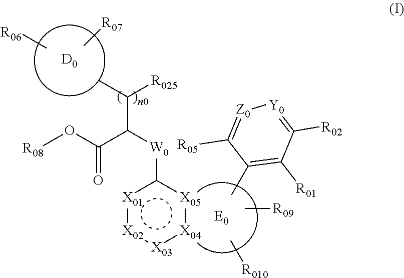
or an enantiomer, diastereoisomer, atropisomer, deuterated derivative, and/or pharmaceutically acceptable salt of any of the foregoing, wherein:
Ring D₀ is a cycloalkyl group, a heterocycloalkyl group, an aryl group or a heteroaryl group,
Ring E₀ is a furyl, thienyl or pyrrolyl ring,
X₀₁, X₀₃, X₀₄ and X₀₅, independently of one another, are a carbon atom or a nitrogen atom,
X₀₂ is a C-R₀₂₆ group or a nitrogen atom, 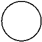
means that the ring is aromatic,
Y₀ is a nitrogen atom or a C-R₀₃ group,
Z₀ is a nitrogen atom or a C-R₀₄ group,
R₀₁ is a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl group, a hydroxy group, a hydroxy(C₁-C₆)alkyl group, a linear or branched (C₁-C₆)alkoxy group, —S—(C₁-C₆)alkyl group, a cyano group, a nitro group, —Cy₀₈, —(C₁-C₆)alkyl-NR₀₁₁R₀₁₁′, —O—(C₁-C₆)alkyl-NR₀₁₁R₀₁₁′, —O—(C₁-C₆)alkyl-R₀₁₂, —C(O)—OR₀₁₁, —O—C(O)—R₀₁₁, —C(O)—NR₀₁₁R₀₁₁′, —NR₀₁₁—C(O)—R₀₁₁′, —NR₀₁₁—C(O)—OR₀₁₁′, —(C₁-C₆)alkyl-NR₀₁₁—C(O)—R₀₁₁′, —SO₂—NR₀₁₁R₀₁₁′, or —SO₂—(C₁-C₆)alkyl,
R₀₂, R₀₃, R₀₄ and R₀₅, independently of one another, are a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl, a hydroxy group, a hydroxy(C₁-C₆)alkyl group, a linear or branched (C₁-C₆)alkoxy group, a —S—(C₁-C₆)alkyl group, a cyano group, a nitro group, —(C₁-C₆)alkyl-NR₀₁₁R₀₁₁′, —O—Cy₀₁, —(C₁-C₆)alkyl-Cy₀₁, —(C₂-C₆)alkenyl-Cy₀₁, —(C₂-C₆)alkynyl-Cy₀₁, —O—(C₁-C₆)alkyl-NR₀₁₁R₀₁₁′, —O—(C₁-C₆)alkyl-R₀₁₃, —O—(C₁-C₆)alkyl-R₀₁₂, —C(O)—OR₀₁₁, —O—C(O)—R₀₁₁, —C(O)—NR₀₁₁R₀₁₁′, —NR₀₁₁—C(O)—R₀₁₁′, —NR₀₁₁—C(O)—OR₀₁₁′, —(C₁-C₆)alkyl-NR₀₁₁—C(O)—R₀₁₁′, —SO₂—NR₀₁₁R₀₁₁′, or —SO₂-(C₁-C₆)alkyl, or the pair (R₀₁, R₀₂), (R₀₂, R₀₃), (R₀₃, R₀₄), or (R₀₄, R₀₅) together with the carbon atoms to which they are attached form an aromatic or non-aromatic ring containing 5 to 7 ring members, which optionally contains 1 to 3 heteroatoms selected from O, S and N, wherein the resulting ring is optionally substituted by 1 or 2 groups selected from halogen, linear or branched (C₁-C₆)alkyl, (C₁-C₆)alkyl-NR₀₁₁R₀₁₁′, —NR₀₁₃R₀₁₃′, —(C₁-C₆)alkyl-Cy₀₁ or oxo,
R₀₆ and R₀₇, independently of one another, are a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl, a hydroxy group, a linear or branched (C₁-C₆)alkoxy group, a —S—(C₁-C₆)alkyl group, a cyano group, a nitro group, —(C₁-C₆)alkyl-NR₀₁₁R₀₁₁′, —O—(C₁-C₆)alkyl-NR₀₁₁R₀₁₁′, —O-Cy₀₁, —(C₁-C₆)alkyl-Cy₀₁, —(C₂-C₆)alkenyl-Cy₀₁, —(C₂-C₆)alkynyl-Cy₀₁, —O—(C₁-C₆)alkyl-R₀₁₂, —C(O)—OR₀₁₁, —O—C(O)—R₀₁₁, —C(O)—NR₀₁₁R₀₁₁′, —NR₀₁₁—C(O)—R₀₁₁′, —NR₀₁₁—C(O)—OR₀₁₁′, —(C₁-C₆)alkyl-NR₀₁₁—C(O)—R₀₁₁′, —SO₂—NR₀₁₁R₀₁₁′, or —SO₂-(C₁-C₆)alkyl, or the pair (R₀₆, R₀₇), when fused with the two adjacent carbon atoms, together with the carbon atoms to which they are attached form an aromatic or non-aromatic ring containing 5 to 7 ring members, which optionally contains 1 to 3 heteroatoms selected from O, S and N, wherein the resulting ring is optionally substituted by a linear or branched (C₁-C₆)alkyl group,
—NR₀₁₃R₀₁₃′, —(C₁-C₆)alkyl-Cy₀₁ or an oxo,
W₀ is a —CH₂— group, a —NH— group or an oxygen atom,
Ros is a hydrogen atom, a linear or branched (C₁-C₅)alkyl group, a —CHR₀ₐR_{0b} group, an aryl group, a heteroaryl group, an aryl(C₁-C₆)alkyl group, or a heteroaryl(C₁-C₆)alkyl group,
R₀₉ is a hydrogen atom, a linear or branched (C₁-C₆)alkyl group, a linear or branched (C₂-C₆)alkenyl group, a linear or branched (C₂-C₆)alkynyl group, —Cy₀₂, —(C₁-C₆)alkyl-Cy₀₂, —(C₂-C₆)alkenyl-Cy₀₂, —(C₂-C₆)alkynyl-Cy₀₂, —Cy₀₂—Cy₀₃, —(C₂-C₆)alkynyl-O-Cy₀₂, —Cy₀₂-(C₁-C₆)alkyl-O-(C₁-C₆)alkyl-Cy₀₃, a halogen atom, a cyano group, —C(O)—R₀₁₄, or —C(O)—NR₀₁₄R₀₁₄′,
R₀₁₀ is a hydrogen atom, a linear or branched (C₁-C₆)alkyl group, a linear or branched (C₂-C₆)alkenyl group, a linear or branched (C₂-C₆)alkynyl group, an aryl(C₁-C₆)alkyl group, a (C₁-C₆)cycloalkylalkyl group, a linear or branched (C₁-C₆)haloalkyl, or -(C₁-C₆)alkyl-O-Cy₀₄,
or the pair (R₀₉, R₀₁₀), when fused with the two adjacent carbon atoms, together with the carbon atoms to which they are attached form an aromatic or non-aromatic ring containing 5 to 7 ring members, which optionally contains 1 to 3 heteroatoms selected from O, S and N,
R₀₁₁ and R₀₁₁′, independently of one another, are a hydrogen atom, an optionally substituted linear or branched (C₁-C₆)alkyl group, or -(C₁-C₆)alkyl-Cy₀₁, or the pair (R₀₁₁, R₀₁₁′) together with the nitrogen atom to which they are attached form an aromatic or non-aromatic ring containing 5 to 7 ring members, which optionally contains, in addition to the nitrogen atom, 1 to 3 heteroatoms selected from O, S, and N, wherein the N atom may be substituted by 1 or 2 groups selected from a linear or branched (C₁-C₆)alkyl group, and wherein one or more of the carbon atoms of the linear or branched (C₁-C₆)alkyl group is optionally deuterated,
R₀₁₂ is —Cy₀₈, —Cy₀₅-(C₁-C₆)alkyl-O-(C₁-C₆)alkyl-Cy₀₆, —Cy₀₅-(C₁-C₆)alkyl-Cy₀₆, —Cy₀₅-(C₁-C₆)alkyl-NR₀₁₁-(C₁-C₆)alkyl-Cy₀₆, —Cy₀₅—Cy₀₆-O-(C₁-C₆)alkyl-Cy₀₇, —Cy₀₅-(C₀-C₆)alkyl-O-(C₁-C₆)alkyl-Cy₀₉, —Cy₀₅-(C₁-C₆)alkyl-Cy₀₉, —NH—C(O)—NH-R₀₁₁, —Cy₀₅-(C₁-C₆)alkyl-NR₀₁₁-(C₁-C₆)alkyl-Cy₀₉, —C(O)—NR₀₁R₀₁₁′, —NR₀₁₁R₀₁₁′, —OR₀₁₁, —NR₀₁₁—C(O)—R₀₁₁′, —O—(C₁-C₆)alkyl-OR₀₁₁, —SO₂-R₀₁₁, —C(O)—OR₀₁₁,
R₀₁₃, R₀₁₃′, R₀₁₄ and R₀₁₄′, independently of one another, are a hydrogen atom, or an optionally substituted linear or branched (C₁-C₆)alkyl group,
R₀ₐ is a hydrogen atom or a linear or branched (C₁-C₆)alkyl group,
R_{0b} is a —O—C(O)—O-R_{0c} group, a —O—C(O)—NR_{0c}R_{0c}′ group, or a —O—P(O)(OR_{0c})₂ group,
R_{0c} and R_{0c}′, independently of one another, are a hydrogen atom, a linear or branched (C₁-C₅)alkyl group, a cycloalkyl group, a (C₁-C₆)alkoxy(C₁-C₆)alkyl group, or a (C₁-C₆)alkoxycarbonyl(C₁-C₆)alkyl group, or the pair (R_{0c}, R_{0c}′) together with the nitrogen atom to which they are attached form a non-aromatic ring composed of from 5 to 7 ring members, which may contain in addition to the nitrogen atom from 1 to 3 heteroatoms selected from oxygen and nitrogen, wherein the nitrogen is optionally substituted by a linear or branched (C₁-C₆)alkyl group,
Cy₀₁, Cy₀₂, Cy₀₃, Cy₀₄, Cy₀₅, Cy₀₆, Cy₀₇, Cy₀₈ and Cy₀₁₀, independently of one another, are a cycloalkyl group, a heterocycloalkyl group, an aryl group or a heteroaryl group, each of which is optionally substituted,
Cy₀₉ is 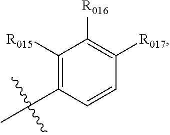
or Cy₀₉ is a heteroaryl group which is substituted by a group selected from —O—P(O)(OR₀₂₀)₂; —O—P(O)(O⁻M⁺)₂; —(CH₂)ₚ₀—O—(CHR₀₁₈—CHR₀₁₉—O)_{q0}—R₀₂₀; hydroxy; hydroxy(C₁-C₆)alkyl; —(CH₂)ᵣ₀-U₀—(CH₂)ₛ₀-heterocycloalkyl; and -U₀—(CH₂)_{q0}-NR₀₂₁R₀₂1′,
R₀₁₅ is a hydrogen atom; a —(CH₂)ₚ₀—O—(CHR₀₁₈—CHR₀₁₉—O)_{q0}—R₀₂₀ group; a linear or branched (C₁-C₆)alkoxy(C₁-C₆)alkyl group; a -U₀—(CH₂)_{q0}-NR₀₂₁R₀₂₁′ group; or a —(CH₂)ᵣ₀-U₀—(CH₂)so-heterocycloalkyl group,
R₀₁₆ is a hydrogen atom; a hydroxy group; a hydroxy(C₁-C₆)alkyl group; a —(CH₂)ᵣ₀-U₀—(CH₂)ₛ₀-heterocycloalkyl group; a (CH₂)ᵣ₀-U₀-V₀-O—P(O)(OR₂₀)₂ group; a -O—P(O)(O⁻M⁺)₂ group; a —O—S(O)₂OR₀₂₀ group; a —S(O)₂OR₀₂₀ group; a —(CH₂)ₚ₀—O—(CHR₀₁₈—CHR₀₁₉—O)_{q0}—R₀₂₀ group; a —(CH₂)ₚ₀—O—C(O)—NR₀₂₂R₀₂₃ group; or a -U₀—(CH₂)_{q0}-NR₀₂1R₀₂1′ group,
R₀₁₇ is a hydrogen atom; a —(CH₂)ₚ₀—O—(CHR₀₁₈—CHR₀₁₉—O)_{q0}—R₀₂₀ group; a —CH₂—P(O)(OR₀₂₀)₂ group, a —O—P(O)(OR₀₂₀)₂ group; a —O—P(O)(O⁻M⁺)₂ group; a hydroxy group; a hydroxy(C₁-C₆)alkyl group; a —(CH₂)ᵣ₀-U₀—(CH₂)so-heterocycloalkyl group; a -U₀—(CH₂)_{q0}-NR₀₂₁R₀₂₁′ group; or an aldonic acid,
M⁺ is a pharmaceutically acceptable monovalent cation,
U₀ is a bond or an oxygen atom,
Vₒ is a —(CH₂)ₛ₀— group or a —C(O)— group,
R₀₁₈ is a hydrogen atom or a (C₁-C₆)alkoxy(C₁-C₆)alkyl group,
R₀₁₉ is a hydrogen atom or a hydroxy(C₁-C₆)alkyl group,
R₀₂o is a hydrogen atom or a linear or branched (C₁-C₆)alkyl group,
R₀₂1 and R₀₂1′ independently of one are a hydrogen atom, a linear or branched (C₁-C₆)alkyl group, or a hydroxy(C₁-C₆)alkyl group, or the pair (R₀₂₁, R₀₂₁′) together with the nitrogen atom to which they are attached form an aromatic or non-aromatic ring containing 5 to 7 ring members, which optionally contains, in addition to the nitrogen atom, 1 to 3 heteroatoms selected from O, S and N, wherein the resulting ring is optionally substituted by a hydrogen atom or a linear or branched (C₁-C₆)alkyl group,
R₀₂₂ is a (C₁-C₆)alkoxy(C₁-C₆)alkyl group, a —(CH₂)ₚ₀-NR₀₂₄R₀₂₄′ group, or a —(CH₂)ₚ₀—O—(CHR₀₁₈—CHR₀₁₉—O)_{q0}—R20 group,
R₀₂₃ is a hydrogen atom or a (C₁-C₆)alkoxy(C₁-C₆)alkyl group, or the pair (R₀₂₂, R₀₂₃) together with the nitrogen atom to which they are attached form an aromatic or non-aromatic ring containing 5 to 18 ring members, which optionally contains, in addition to the nitrogen atom, 1 to 5 heteroatoms selected from O, S and N, wherein the resulting ring is optionally substituted by a hydrogen atom, a linear or branched (C₁-C₆)alkyl group or a heterocycloalkyl group,
R₀₂₄ and R₀₂₄′, independently of one another, are a hydrogen atom or a linear or branched (C₁-C₆)alkyl group, or the pair (R₀₂₄, R₀₂₄′) together with the nitrogen atom to which they are attached form an aromatic or non-aromatic ring composed of from 5 to 7 ring members, which may contain in addition to the nitrogen atom from 1 to 3 heteroatoms selected from O, S and N, and wherein the resulting ring is optionally substituted by a hydrogen atom or a linear or branched (C₁-C₆)alkyl group,
R₀₂₅ is a hydrogen atom, a hydroxy group, or a hydroxy(C₁-C₆)alkyl group,
R₀₂₆ is a hydrogen atom, a halogen atom, a linear or branched (C₁-C₆)alkyl group, or a cyano group,
R₀₂₇ is a hydrogen atom or a linear or branched (C₁-C₆)alkyl group,
R₀₂8 is a —O—P(O)(O⁻)(O⁻) group, a —O—P(O)(O⁻)(OR₀₃O) group, a —O—P(O)(OR₀₃₀)(OR₀₃O′) group, a —(CH₂)ₚ₀—O—SO₂—O— group, a —(CH₂)ₚ₀—SO₂—O— group, a —(CH₂)ₚ₀—O—SO₂—OR₀₃₀ group, —Cy₀₁₀, a —(CH₂)ₚ₀—SO₂—OR₀₃₀ group, a —O—C(O)—R₀₂₉ group, a —O—C(O)—OR₀₂₉ group or a —O—C(O)—NR₀₂₉R₀₂₉′ group;
R₀₂₉ and R₀₂₉′, independently of one another, are a hydrogen atom, a linear or branched (C₁-C₆)alkyl group or a linear or branched amino(C₁-C₆)alkyl group,
R₀₃₀ and R₀₃O′, independently of one another, are a hydrogen atom, a linear or branched (C₁-C₆)alkyl group or an aryl(C₁-C₆)alkyl group,
R₀₃₁ is 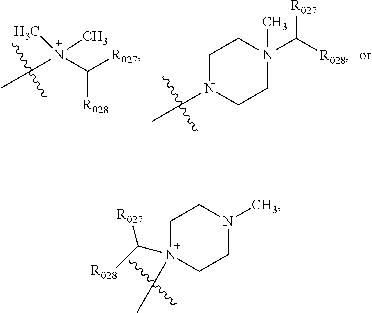
wherein the ammonium optionally exists as a zwitterionic form or has a monovalent anionic counterion,
n₀ is an integer equal to 0 or 1,
p₀ is an integer equal to 0, 1, 2, or 3,
q₀ is an integer equal to 1, 2, 3 or 4,
r₀ and so are independently an integer equal to 0 or 1;
wherein, at most, one of the R₀₃, R₀₉, or R₀₁₂ groups, if present, is covalently attached to the linker, and
wherein the valency of an atom is not exceeded by virtue of one or more substituents bonded thereto; or
(ii) Formula (IA): 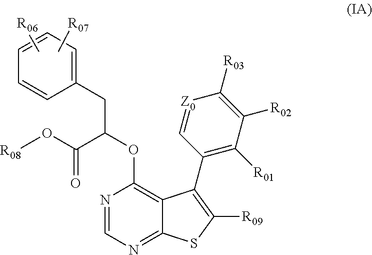
or an enantiomer, diastereoisomer, atropisomer, deuterated derivative, and/or pharmaceutically acceptable salt of any of the foregoing,
wherein:
Z₀ is a nitrogen atom or a C-R₀₄ group,
R₀₁ is a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl group, a hydroxy group, a linear or branched (C₁-C₆)alkoxy group, a —S—(C₁-C₆)alkyl group, a cyano group, —Cy₀₈, —NR₀₁₁R₀₁₁′,
R₀₂, R₀₃ and R₀₄, independently of one another, are a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl, a hydroxy group, a linear or branched (C₁-C₆)alkoxy group, a —S—(C₁-C₆)alkyl group, a cyano group, a nitro group, —(C₁-C₆)alkyl-NR₀₁₁R₀₁₁′, —O—Cy₀₁, —(C₁-C₆)alkyl-Cy₀₁, —(C₂-C₆)alkenyl, —(C₂-C₆)alkynyl, —O—(C₁-C₆)alkyl-NR₀₁R₀₁₁′, —O—(C₁-C₆)alkyl-R₀₃₁, —C(O)—OR₀₁₁, —O—C(O)—R₀₁₁, —C(O)—NR₀₁₁R₀₁₁′, —NR₀₁₁—C(O)—R₀₁₁′, —NR₀₁₁—C(O)—OR₀₁₁′, —(C₁-C₆)alkyl-NR₀₁₁—C(O)—R₀₁₁′, —SO₂—NR₀₁₁R₀₁₁′, or —SO₂-(C₁-C₆)alkyl, or the pair (R₀₂, R₀₃) or (R₀₃, R₀₄) together with the carbon atoms to which they are attached form an aromatic or non-aromatic ring containing 5 to 7 ring members, which optionally contains 1 to 3 heteroatoms selected from O, S and N, wherein the ring is optionally substituted by a group selected from a linear or branched (C₁-C₆)alkyl, —NR₀₁₃R₀₁₃′, —(C₁-C₆)alkyl-Cy₀₁ and oxo,
R₀₆ and R₀₇, independently of one another, are a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)haloalkyl, a hydroxy group, a linear or branched (C₁-C₆)alkoxy group, a —S—(C₁-C₆)alkyl group, a cyano group, a nitro group, —(C₀-C₆)alkyl-NR₀₁₁R₀₁₁′, —O—Cy₀₁, —(C₁-C₆)alkyl-Cy₀₁, —(C₂-C₆)alkenyl-Cy₀₁, —(C₂-C₆)alkynyl-Cy₀₁, —O—(C₁-C₆)alkyl-R₀₁₂, —C(O)—OR₀₁₁, —O—C(O)—R₀₁₁, —C(O)—NR₀₁R₀₁₁′, —NR₀₁₁—C(O)—R₀₁₁′, —NR₀₁₁—C(O)—OR₀₁₁′, —(C₁-C₆)alkyl-NR₀₁-C(O)—R₀₁′, —SO₂—NR₀₁R₀₁₁′, or —SO₂-(C₁-C₆)alkyl, or the pair (R₀₆, R₀₇), when fused with two adjacent carbon atoms, together with the carbon atoms to which they are attached form an aromatic or non-aromatic ring containing 5 to 7 ring members, which optionally contains 1 to 3 heteroatoms selected from O, S and N, and wherein the resulting ring is optionally substituted by a group selected from a linear or branched (C₁-C₆)alkyl group, —NR₀₁₃R₀₁₃′, —(C₁-C₆)alkyl-Cy₀₁ and an oxo,
R₀₈ is a hydrogen atom, a linear or branched (C₁-C₈)alkyl group, an aryl group, a heteroaryl group, an aryl-(C₁-C₆)alkylgroup, or a heteroaryl(C₁-C₆)alkyl group,
R₀₉ is a linear or branched (C₁-C₆)alkyl group, a linear or branched (C₂-C₆)alkenyl group, a linear or branched (C₂-C₆)alkynyl group, —Cy₀₂, —(C₁-C₆)alkyl-Cy₀₂, —(C₂-C₆)alkenyl-Cy₀₂, —(C₂-C₆)alkynyl-Cy₀₂, —Cy₀₂—Cy₀₃, —(C₂-C₆)alkynyl-O-Cy₀₂, —Cy₀₂-(C₁-C₆)alkyl-O-(C₁-C₆)alkyl-Cy₀₃, a halogen atom, a cyano group, —C(O)—R₀₁₄, —C(O)—NR₀₁₄R₀₁₄′, R₀₁₁ and R₀₁₁′, independently of one another, are a hydrogen atom, an optionally substituted linear or branched (C₁-C₆)alkyl group, or -(C₁-C₆)alkyl-Cy₀₁, or the pair (R₀₁₁, R₀₁₁′) together with the nitrogen atom to which they are attached form an aromatic or non-aromatic ring containing 5 to 7 ring members, which optionally contains, in addition to the nitrogen atom, 1 to 3 heteroatoms selected from O, S and N, wherein the N atom is optionally substituted by a linear or branched (C₁-C₆)alkyl group, and wherein one or more of the carbon atoms of the linear or branched (C₁-C₆)alkyl group is optionally deuterated,
R₀₁₂ is —Cy₀₅, —Cy₀₅-(C₁-C₆)alkyl-Cy₀₆, —Cy₀₅-(C₁-C₆)alkyl-O-(C₁-C₆)alkyl-Cy₀₆, —Cy₀₅-(C₁-C₆)alkyl-NR₀₁₁-(C₁-C₆)alkyl-Cy₀₆, —Cy₀₅—Cy₀₆—O-(C₁-C₆)alkyl-Cy₀₇, —Cy₀₅-(C₀-C₆)alkyl-Cy₀₉, —NH—C(O)—NH-R₀₁₁, —C(O)—NR₀₁R₀₁₁′, —NR₀₁₁R₀₁₁′, —OR₀₁₁, —NR₀₁₁—C(O)—R₀₁₁′, —O—(C₁-C₆)alkyl-OR₀₁₁, —SO₂—R₀₁₁, or —C(O)—OR₀₁₁,
R₀₁₂ R₀₁₃′, R₀₁₄ and R₀₁₄′, independently of one another, are a hydrogen atom, or an optionally substituted linear or branched (C₁-C₆)alkyl group,
Cy₀₁, Cy₀₂, Cy₀₃, Cy₀₅, Cy₀₆, Cy₀₇ and Cy₀₈, independently of one another, are a cycloalkyl group, a heterocycloalkyl group, an aryl group or a heteroaryl group, each of which is optionally substituted,
Cy₀₉ is 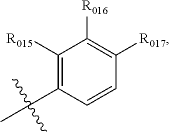
wherein R₀₁₅, R₀₁₆, and R₀₁₇ are as defined for formula (I),
R₀₃₁ is 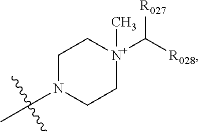
wherein R₀₂₇ an R₀₂₈ are defined form formula (I)
wherein, at most, one of the R₀₃, R₀₉, or R₀₁₂ groups, if present, is covalently attached to the linker; or
(iii) Formula (IB): 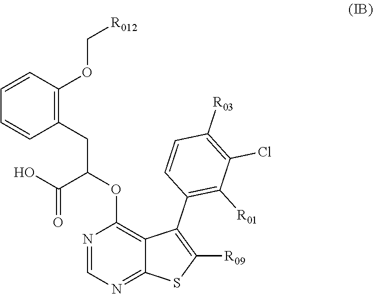
or the enantiomer, diastereoisomer, atropisomer, deuterated derivative, and/or
pharmaceutically acceptable salt of any of the foregoing
wherein:
R₀₁ is a linear or branched (C₁-C₆)alkyl group,
R₀₃ is —O-(C₁-C₆)alkyl-NR₀₁₁R₀₁₁′, or 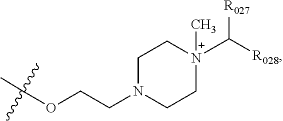
wherein R₀₁₁ and R₀₁₁′, independently of one another, are a hydrogen atom, an optionally substituted linear or branched (C₁-C₆)alkyl group, or -(C₁-C₆)alkyl-Cy₀₁;
or the pair (R₀₁₁, R₀₁₁′) together with the nitrogen atom to which they are attached form an aromatic or non-aromatic ring containing 5 to 7 ring members, which optionally contains, in addition to the nitrogen atom, 1 to 3 heteroatoms selected from O, S and N, wherein the N atom may be substituted by 1 or 2 groups selected from a hydrogen atom or a linear or branched (C₁-C₆)alkyl group,
and wherein R₀₂₇ is a hydrogen atom and R₀₂8 is a —(CH₂)ₚ₀—O—SO₂—O— group or a —(CH₂)ₚ₀—SO₂—OR₀₃₀ group;
R₀₉ is a linear or branched (C₂-C₆)alkynyl group or —Cy₀₂,
R₀₁₂ is —Cy₀₅, —Cy₀₅-(C₁-C₆)alkyl-Cy₀₆, or —Cy₀₅-(C₁-C₆)alkyl-Cy₀₉,
Cy₀₁, Cy₀₂, Cy₀₅ and Cy₀₆ independently of one another, are a cycloalkyl group, a heterocycloalkyl group, an aryl group or a heteroaryl group, each of which is optionally substituted,
Cy₀₉ is 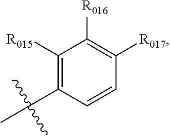
R₀₁₅, R₀₁₆, and R₀₁₇ are as defined for formula (I),
wherein, at most, one of the R₀₃, Rog, or R₀₁₂ groups, if present, is covalently attached to the linker.

90. The antibody-drug conjugate of claim 89, wherein:
(i) Cy₀₁, Cy₀₂, Cy₀₃, Cy₀₄, Cy₀₅, Cy₀₆, Cy₀₇, Cy₀₈ and Cy₀₁₀, independently of one another, is a cycloalkyl group, a heterocycloalkyl group, an aryl group or a heteroaryl group, each of which is optionally substituted by one or more groups selected from halo; -(C₁-C₆)alkoxy; -(C₁-C₆)haloalkyl; -(C₁-C₆)haloalkoxy; —(CH₂)po-O—SO₂—OR₀₃O; —(CH₂)ₚ₀—SO₂—OR₀₃₀; —O—P(O)(OR₀₂₀)₂; —O—P(O)(O⁻M⁺)₂; —CH₂—P(O)(OR₀₂₀)₂; —(CH₂)ₚ₀—O—(CHR₀₁₈—CHR₀₁₉—O)_{q0}—R₂₀; hydroxy; hydroxy(C₁-C₆)alkyl; —(CH₂)ᵣ₀-U₀—(CH₂)so-heterocycloalkyl; or -U₀—(CH₂)_{q0}-NR₀₂₁R₀₂₁′;
(ii) R₀₁ is methyl or ethyl;
(iii) R₀₃ is —O—CH₂—CH₂—NR₀₁₁R₀₁₁′ in which R₀₁₁ and R₀₁₁′ form, together with the nitrogen atom carrying them, a piperazinyl group which may be substituted by a group being a hydrogen atom or a linear or branched (C₁-C₆)alkyl group
(iv) R₀₃ comprises the formula: 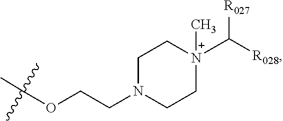
wherein
R₀₂₇ is a hydrogen atom and R₀₂8 is a —(CH₂)no-SO₂—OR₀₃>group;
(v) R₀₃ comprises the formula: 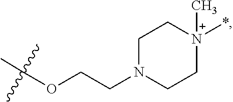
wherein [symbol] is a bond to the linker;
(vi) R₀₉ is Cy₀₂; optionally wherein Cy₀₂ is an optionally substituted aryl group;
(vii) Cy₀₅ comprises a heteroaryl group selected from a pyrazolyl group and a pyrimidinyl group;
(viii) Cy₀₅ is a pyrimidinyl group; and/or
(ix) the Mcl-1 inhibitor is attached by a covalent bond to R₀₃ of formula (I), (IA), or (IB);
or is attached by a covalent bond to R₀₉ of formula (I), (IA), or (IB).

91-101. (canceled)

102. The antibody-drug conjugate of claim 89, wherein the Mcl-1 inhibitor is represented by any one of the following formulas: 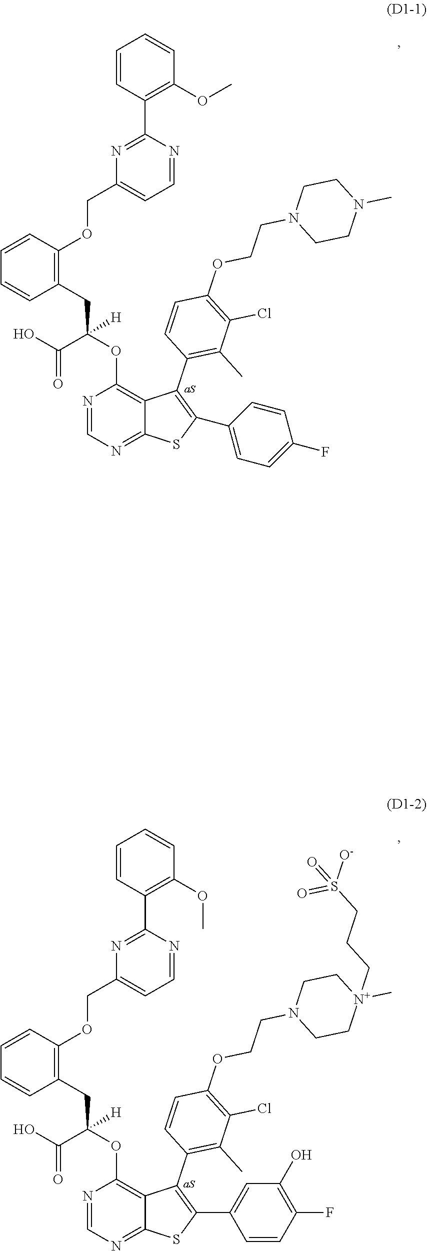 [SMILES: COc1ccccc1-c1nccc(COc2ccccc2C[C@@H](Oc2ncnc3sc(-c4ccc(F)cc4)c(-c4ccc(OCCN5CCN(C)CC5)c(Cl)c4C)c23)C(=O)O)n1 ; COc1ccccc1-c1nccc(COc2ccccc2C[C@@H](Oc2ncnc3sc(-c4ccc(F)c(O)c4)c(-c4ccc(OCCN5CC[N+](C)(CCCS(=O)(=O)[O-])CC5)c(Cl)c4C)c23)C(=O)O)n1] 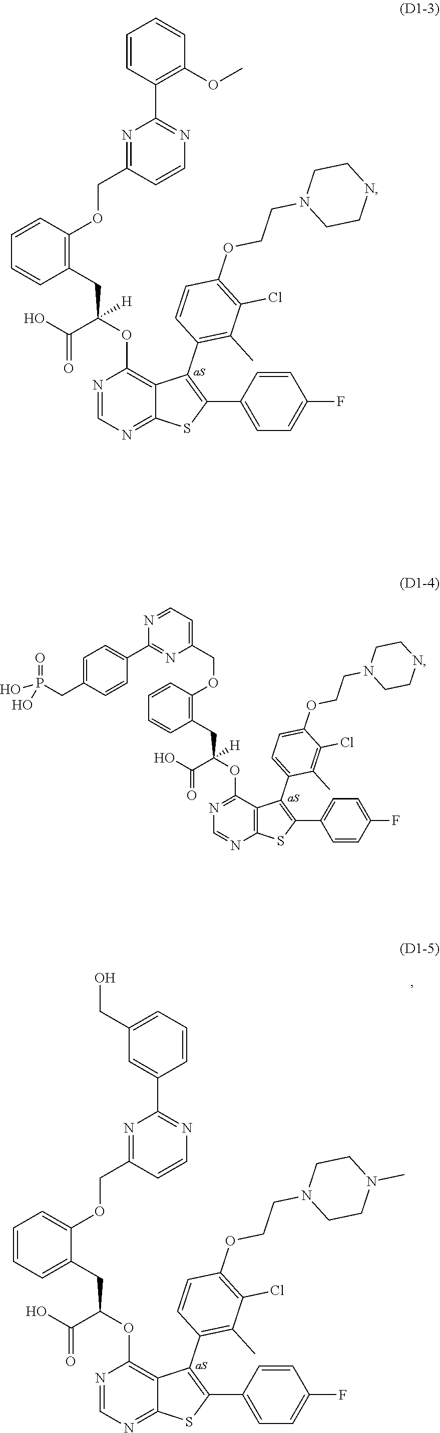 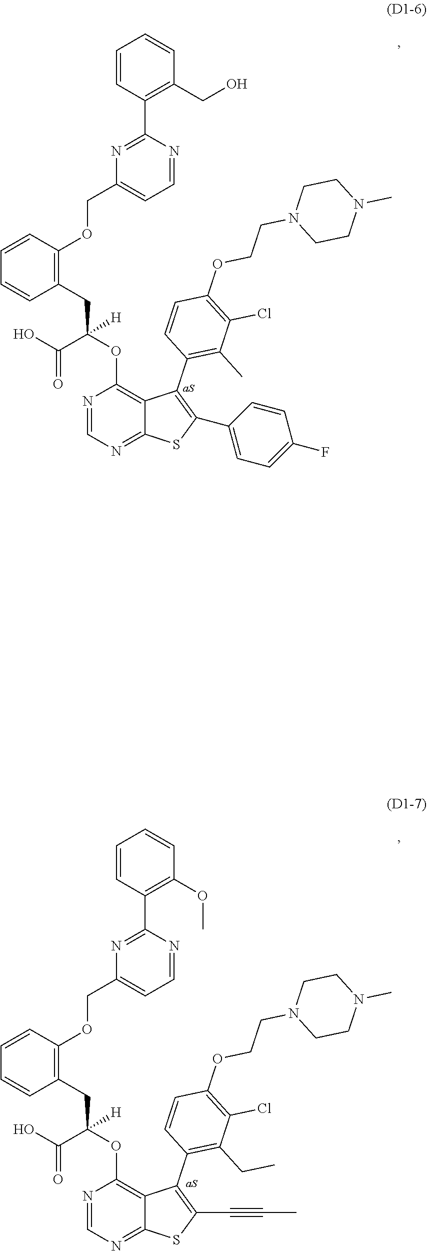 [SMILES: Cc1c(-c2c(-c3ccc(F)cc3)sc3ncnc(O[C@H](Cc4ccccc4OCc4ccnc(-c5ccccc5CO)n4)C(=O)O)c23)ccc(OCCN2CCN(C)CC2)c1Cl ; CC#Cc1sc2ncnc(O[C@H](Cc3ccccc3OCc3ccnc(-c4ccccc4OC)n3)C(=O)O)c2c1-c1ccc(OCCN2CCN(C)CC2)c(Cl)c1CC] 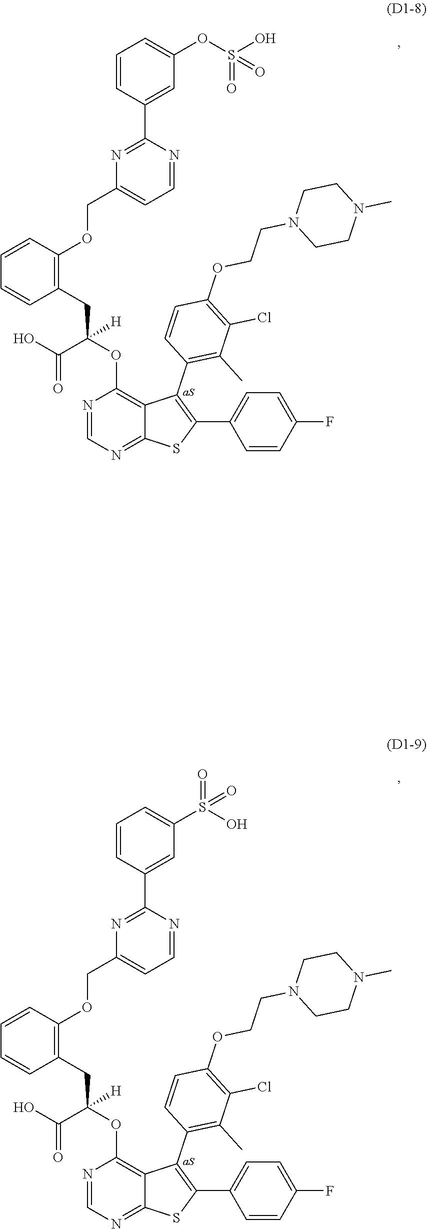 [SMILES: Cc1c(-c2c(-c3ccc(F)cc3)sc3ncnc(O[C@H](Cc4ccccc4OCc4ccnc(-c5cccc(OS(=O)(=O)O)c5)n4)C(=O)O)c23)ccc(OCCN2CCN(C)CC2)c1Cl ; Cc1c(-c2c(-c3ccc(F)cc3)sc3ncnc(O[C@H](Cc4ccccc4OCc4ccnc(-c5cccc(S(=O)(=O)O)c5)n4)C(=O)O)c23)ccc(OCCN2CCN(C)CC2)c1Cl] 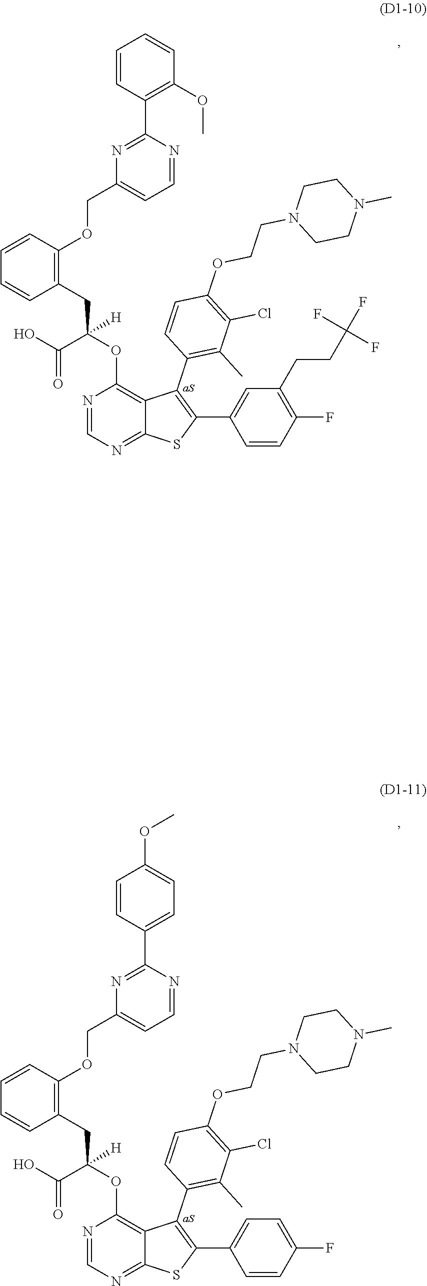 [SMILES: COc1ccccc1-c1nccc(COc2ccccc2C[C@@H](Oc2ncnc3sc(-c4ccc(F)c(CCC(F)(F)F)c4)c(-c4ccc(OCCN5CCN(C)CC5)c(Cl)c4C)c23)C(=O)O)n1 ; COc1ccc(-c2nccc(COc3ccccc3C[C@@H](Oc3ncnc4sc(-c5ccc(F)cc5)c(-c5ccc(OCCN6CCN(C)CC6)c(Cl)c5C)c34)C(=O)O)n2)cc1] 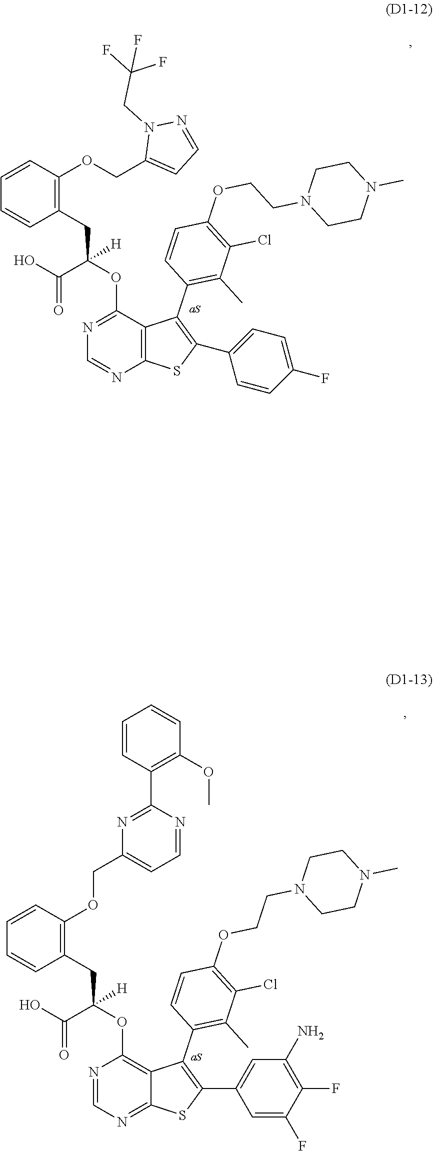 [SMILES: Cc1c(-c2c(-c3ccc(F)cc3)sc3ncnc(O[C@H](Cc4ccccc4OCc4ccnn4CC(F)(F)F)C(=O)O)c23)ccc(OCCN2CCN(C)CC2)c1Cl ; COc1ccccc1-c1nccc(COc2ccccc2C[C@@H](Oc2ncnc3sc(-c4cc(N)c(F)c(F)c4)c(-c4ccc(OCCN5CCN(C)CC5)c(Cl)c4C)c23)C(=O)O)n1] 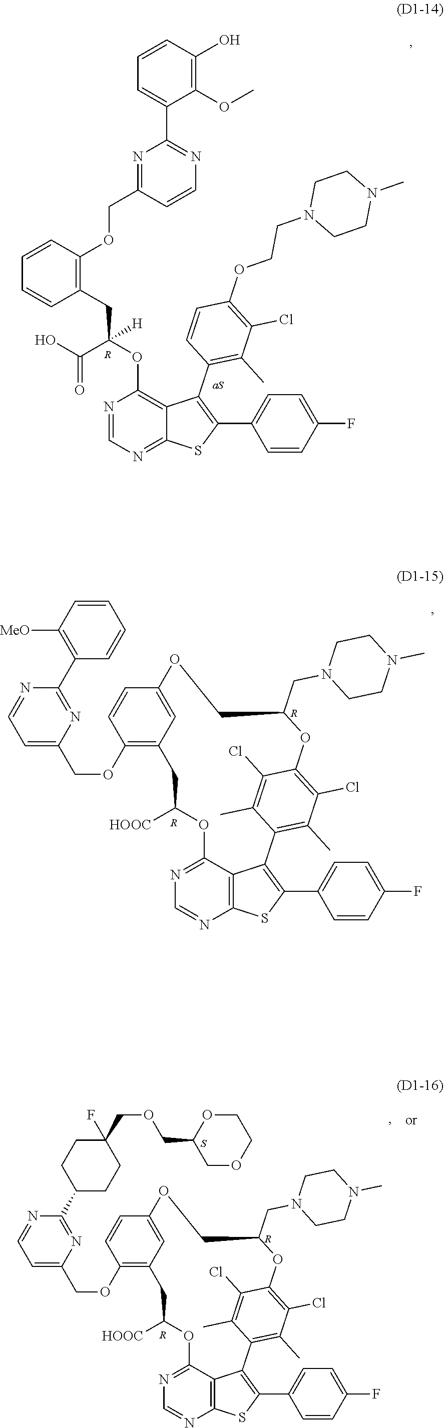 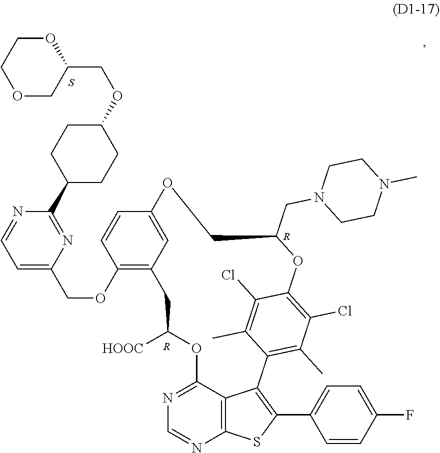
or an enantiomer, diastereoisomer, atropisomer, deuterated derivative, and/or pharmaceutically acceptable salt of any of the foregoing.

103. The antibody-drug conjugate of claim 70, wherein:
ii the Bcl-xL inhibitor is represented by Formula (II) or Formula (III): 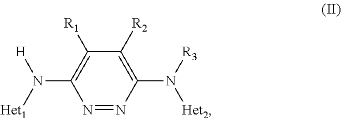
or an enantiomer, a diastereoisomer, and/or a pharmaceutically acceptable salt of any one of the foregoing, wherein:
R₁ and R₂, independently of one another, represent a group selected from the group consisting of: hydrogen; a linear or branched C₁-C₆alkyl optionally substituted by a hydroxyl or a C₁-C₆alkoxy group; a C₃-C₆cycloalkyl; a trifluoromethyl; and a linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from the group consisting of: hydrogen; a C₃-C₆cycloalkyl; a linear or branched C₁-C₆alkyl; -X₁—NRₐR_{b}; -X₁-N+RₐR_{b}R_{c}; -X₁—O-R_{c}; -X₁-COOR_{c}; -X₁-PO(OH)₂; -X₁-SO₂(OH); -X₁-N₃ and: 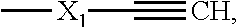
Rₐ and R_{b}, independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O-; a C₁-C₆alkylene-COOH; a C₁-C₆alkylene-PO(OH)₂; a C₁-C₆alkylene-NRdRe; a C₁-C₆alkylene-N+R_{d}RₑR_{f}; a C₁-C₆alkylene-phenyl wherein the phenyl may be substituted by a C₁-C₆alkoxy group; and the group: 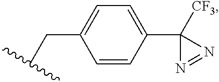 [SMILES: *Cc1ccc(C2(C(F)(F)F)N=N2)cc1]
or Rₐ and R_{b} form with the nitrogen atom carrying them a cycle Bi;
or Rₐ, R_{b} and R_{c} form with the nitrogen atom carrying them a bridged C₃-C₈hetero cycloalkyl,
R_{c}, R_{d}, Rₑ, R_{f}, independently of one another, represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈heterocycloalkyl,
Het₁ represents a group selected from the group consisting of: 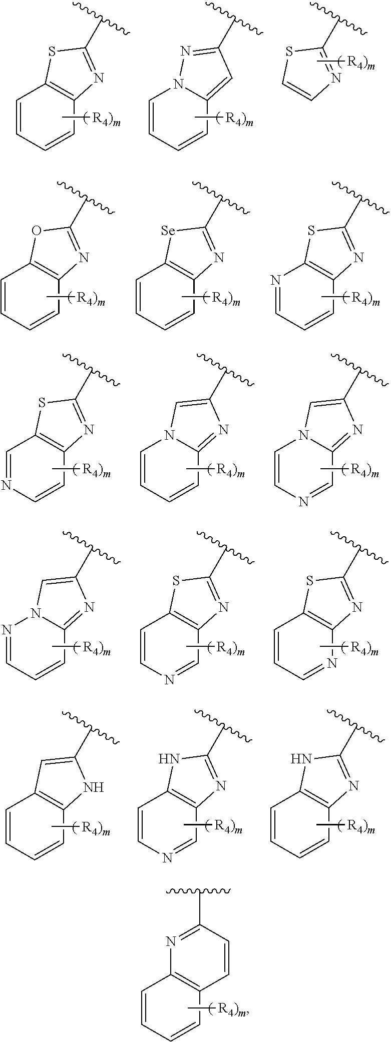
Het₂ represents a group selected from the group consisting of: 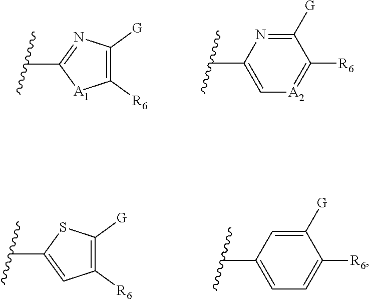
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2},
—NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, -C₁-C₆alkyl optionally substituted by a hydroxyl group, a halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C_{G}alkyl substituted by a hydroxyl, a C₁-C₆alkyl substituted by a C₁-C₆alkoxy group, a C₂-C₆alkenyl, a C₂-C₆alkynyl, a C₃-C₆cycloalkyl, phenyl and —(CH₂)₁₋4-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋4-phenyl; or R_{G1} and R_{G2}, together with the atom to which each is attached are combined to form a C₃-C₆heterocycloalkyl; or in the alternative, G is selected from the group consisting of: 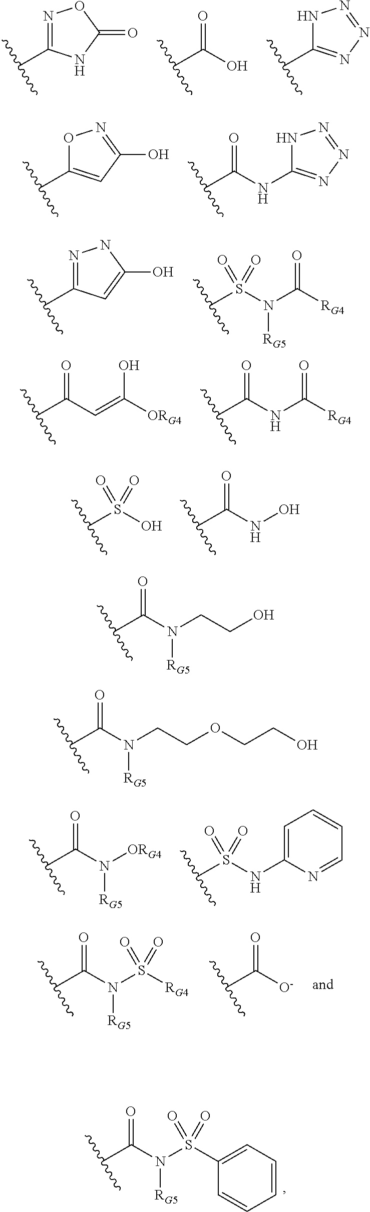
wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆represents a group selected from the group consisting of:
hydrogen;
a linear or branched -C₁-C₆alkylene-R8 group;
a -C₂-C₆alkenyl; —X₂—O—R₇; 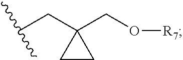
X₂-NSO₂-R₇;
—C═C(R₉)-Y₁-O-R₇;
a C₃-C₆cycloalkyl;
a C₃-C₆heterocycloalkyl optionally substituted by a hydroxyl group;
a C₃-C₆cycloalkylene-Y₂-R₇;
a C₃-C₆heterocycloalkylene-Y₂-R₇ group, and
a heteroarylene-R₇ group optionally substituted by a linear or branched C₁-C₆alkyl group, R₇ represents a group selected from the group consisting of: a linear or branched C₁-C₆alkyl group; a (C₃-C₆)cycloalkylene-R₈; 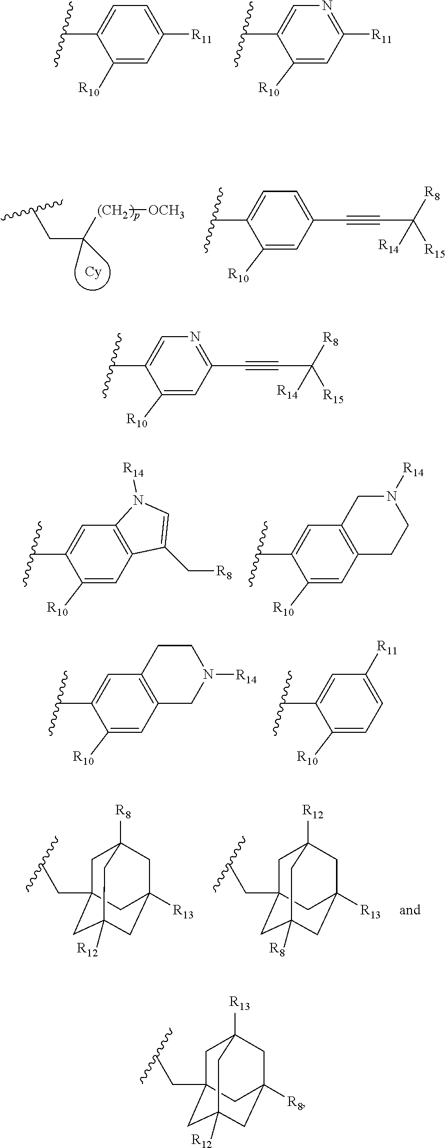
wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐCO—R′_{c}; —NR′ₐR′_{b}R′_{c}—O—R′_{c}; —NH— X′₂—N⁺R′ₐR′bR′_{c}; —O—X′₂—NR′ₐR′_{b}; —X′₂—NR′ₐR′_{b}; —NR′_{c}—X′₂—N₃ and 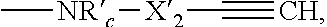
R₉ represents a group selected from the group consisting of a linear or branched C₁-C₆alkyl, trifluoromethyl, hydroxyl, halogen, and a C₁-C₆alkoxy,
Rio represents a group selected from the group consisting of hydrogen, fluorine, chlorine, bromine, —CF₃ and methyl,
R₁₁ represents a group selected from the group consisting of hydrogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂-R₈, and a C₃-C₈heterocycloalkylene-CH₂-R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂, independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b}, independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′f; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 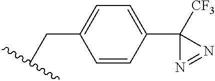 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈hetero cycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄, or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—,-C₂H₅-, —CO—, —CO—O—, —CO—CH₂—, —CO—NH—CH₂—, —SO₂—, —SO₂—CH₂—, —NH—CO—, or -NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl, wherein one of the R₃ and R₈ groups, if present, is covalently attached to the linker, and wherein the valency of an atom is not exceeded by virtue of one or more substituents bonded thereto; or 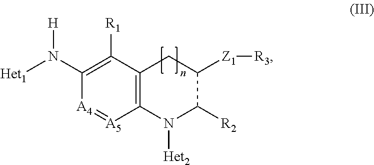
or an enantiomer, a diastereoisomer, and/or a pharmaceutically acceptable salt of any one of the foregoing, wherein:
n=0, 1 or 2, [symbol] [symbol]
represents a single or a double bond,
A₄ and A₅, independently of one another, represent a carbon or a nitrogen atom,
Zi represents a bond, —N(R)—, or —O—, wherein R represents a hydrogen or a linear or branched C₁-C₆alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
R₂represents a hydrogen or a methyl;
R₃ represents a group selected from the group consisting of: hydrogen; a linear or branched C₁-C₄alkyl; —X₁—NRₐR_{b}; —X₁—N⁺RₐR_{b}R_{c}; —X₁-O—R_{c}; —X₁-COOR_{c}; —X₁-PO(OH)₂; —X₁-SO₂(OH); —X₁-N₃ and: 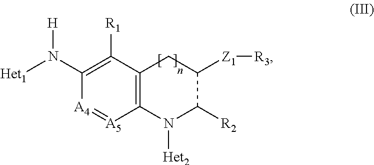
Rₐ and R_{b}, independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O⁻; a C₁-C₆alkylene-COOH; a C₁-C₆alkylene-PO(OH)₂; a C₁-C₆alkylene-NRdRe; a C₁-C₆alkylene-N⁺R_{d}RₑR_{f}; a C₁-C₆alkylene-phenyl wherein the phenyl may be substituted by a C₁-C₆alkoxy group; and the group: 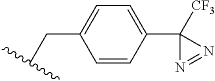 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another, represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈ heterocycloalkyl,
Het₁ represents a group selected from the group consisting of: 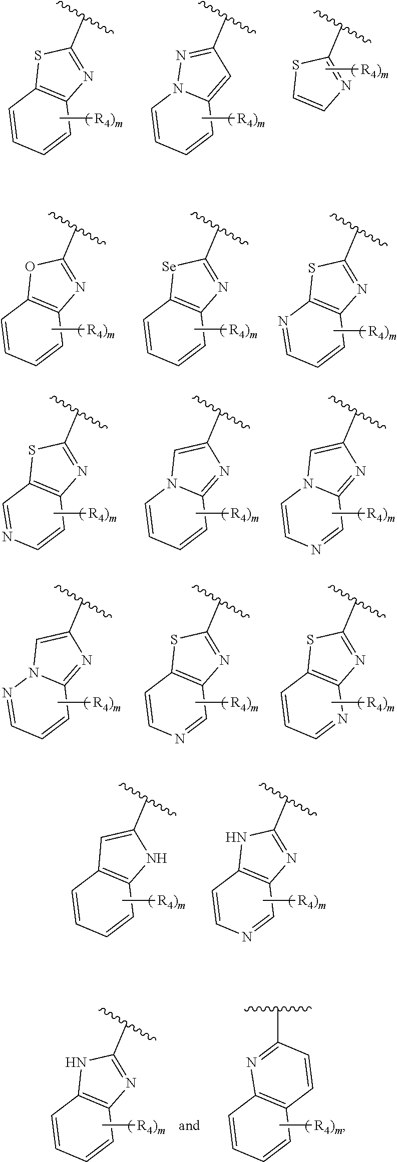
Het₂ represents a group selected from the group consisting of: 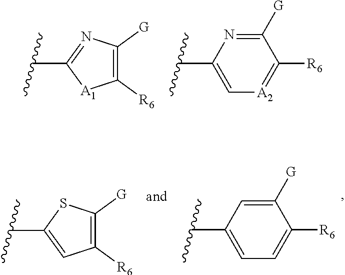
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, -C₁-C₆alkyl optionally substituted by a hydroxyl group, halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆ alkynyl, a C₃-C₆cycloalkyl, phenyl and —(CH₂)1-₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋4-phenyl; or R_{G1} and R_{G2}, together with the atom to which each is attached are combined to form a C₃-C₈heterocycloalkyl; or in the alternative, G is selected from the group consisting of: 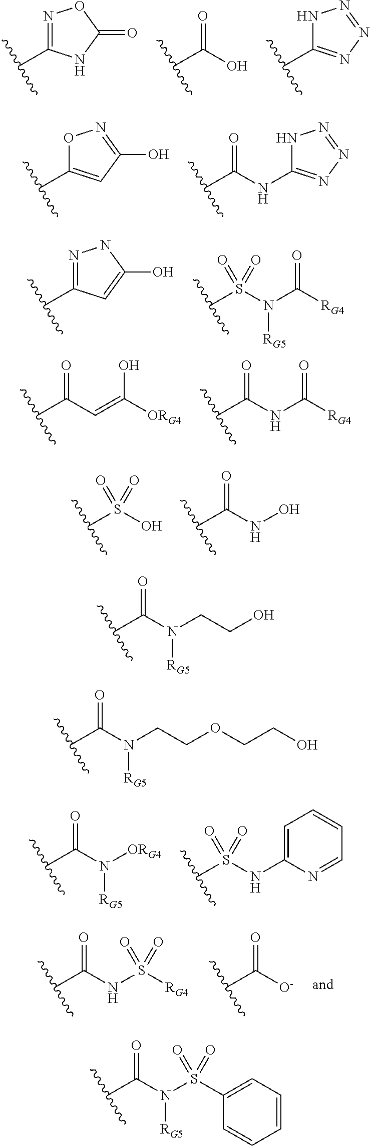
wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆ alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆represents a group selected from the group consisting of: hydrogen;
a linear or branched -C₁-C₆alkylene-R₈ group;
a -C₂-C₆alkenyl;
—X₂—O—R₇; 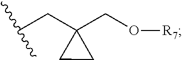
—X₂—NSO₂-R₇;
—C═C(R₉)-Y₁-O—R₇;
a C₃-C₆cycloalkyl;
a C₃-C₆heterocycloalkyl optionally substituted by a hydroxyl group;
a C₃-C₆cycloalkylene-Y₂-R₇;
a C₃-C₆heterocycloalkylene-Y₂-R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆)cycloalkylene-R₈; 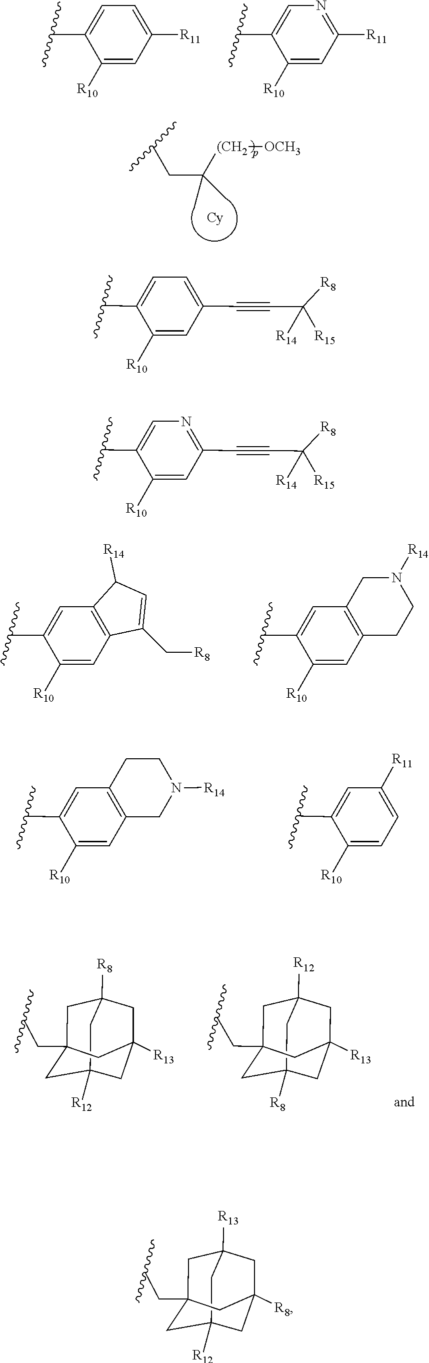
wherein Cy represents a C₃-C₈cycloalkyl,
R₈ represents a group selected from the group consisting of: hydrogen; a linear or branched C₁-C₆alkyl, —NR′ₐR′_{b}; —NR′ₐCO—OR′_{c}; —NR′ₐCO—R′_{c}; —N⁺R′ₐbR′_{c}—O—R′_{c}; NH—X′₂—N⁺R′ₐR′bR′; —O—X′₂—NR′ₐR′_{b}, —X′₂—NR′ₐR′_{b}, —NR′_{c}—X′₂—N₃ and: 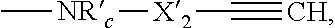
R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂-R₈, and a C₃-C₈heterocycloalkylene-CH₂-R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂represents a linear or branched C₁-C₆alkylene,
R′ₐ and R′_{b}, independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group: 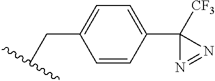 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂−O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl, wherein one of the R₃, Rs and G groups, if present, is covalently attached to the linker, and wherein the valency of an atom is not exceeded by virtue of one or more substituents bonded thereto;
(ii) the Bcl-xL inhibitor is represented by formula (IIA) or (IIIA): 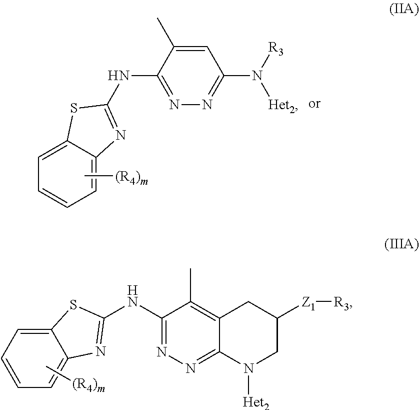
or an enantiomer, a diastereoisomer, and/or a pharmaceutically acceptable salt of any one of the foregoing, wherein:
Z₁ represents a bond or —O—,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—N₃ and 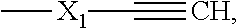
Rₐ and R_{b}, independently of one another, represent a group selected from the group consisting of: hydrogen; a linear or branched C₁-C₆alkyl optionally substituted by one or two hydroxyl groups; and a C₁-C₆alkylene-SO₂O—,
R_{c} represents a hydrogen or a linear or branched C₁-C₆alkyl group,
Het₂ represents a group selected from the group consisting of: 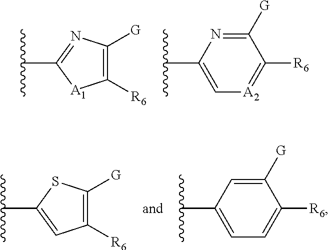
A₁ is —NH—, —N(C₁-C₃alkyl), 0, S or Se,
A₂ is N, CH or C(R₅),
G is selected from the group consisting of:
—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2},
—OC(O)NR_{G1}R_{G2}, —NR_{G1}C(O)OR_{G3}, —C(═NOR_{G1})NR_{G1}R_{G2},
—NR_{G1}C(═NCN)NR_{G1}R_{G2}, —NR_{G1}S(O)₂NR_{G1}R_{G2}, —S(O)₂R_{G3}, —S(O)₂NR_{G1}R_{G2},
—NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, —C(O)NRG₅S(O)₂R_{G4}, halogen, —NO₂, and —CN, in which:
R_{G1}, R_{G2}, R_{G4} and R_{G5} at each occurrence are each independently selected from the group consisting of hydrogen, and a C₁-C₆alkyl optionally substituted by 1 to 3 halogen atoms;
R_{G3} is a C₁-C₆alkyl optionally substituted by 1 to 3 halogen atoms; or
R_{G1} and R_{G2}, together with the atom to which each is attached are combined to form a C₃-C₈heterocycloalkyl;
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a halogen and —CN,
R₆represents a group selected from the group consisting of:
a linear or branched -C₁-C₆alkylene-R₈ group;
—X₂—O—R₇; and
a heteroarylene-R₇ group optionally substituted by a linear or branched C₁-C₆alkyl group,
R₇ represents a group selected from the group consisting of: a linear or branched C₁-C₆alkyl group; (C₃-C₆)cycloalkylene-R₈; 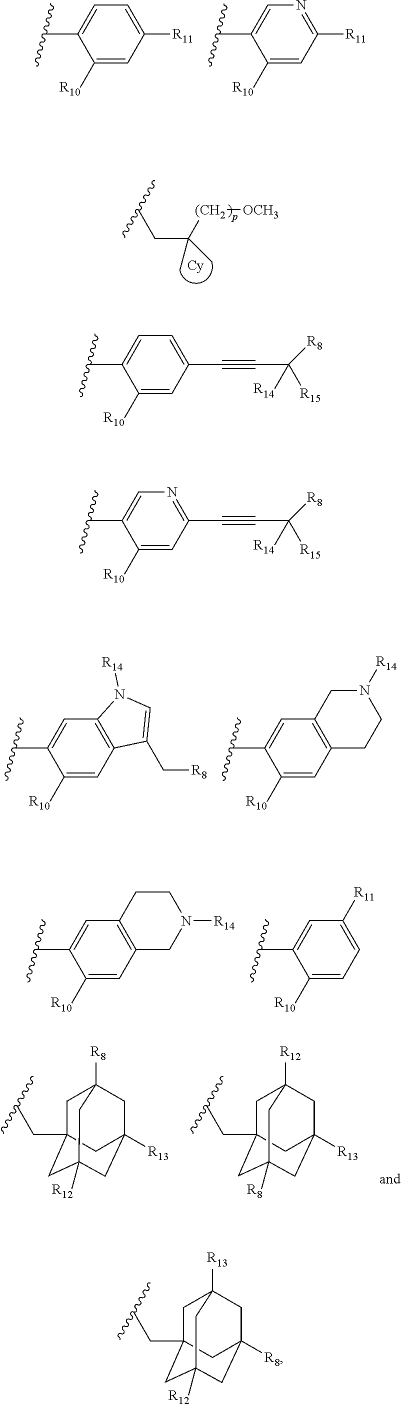
wherein C represents a C₃—C₈yclhclx,,
R₈ represents a group selected from the group consisting of: hydrogen: a linear or branched C₁-C₈alkyl, —NR′ₐR′_{b}: —NR′ₐ—CO—OR′: —NR′ₐ—CO—R′: —N⁺R′ₐRK_{b}R′_{g}—O—R′: —NH—X′₂—N⁺R′ₐR′bR′; OOX′₂—NR′,R′_{b}; —X′₂—NR′,R′_{b}; —NR′,—X′₂—N₃ and: 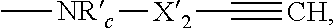
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, —O—
C₁-C₃alkylene-R₈, —CO-NRliRi, —CH═CH—C₁-C₄˜alkylene-NRnR;, —CH═CH—CHO, aC3 Cscy cloalylene-CH₂-RS, and a C₃-Csheterocy cloalylene-CH₂-RS,
R₁₂ and R₁3, independently of one another, represent a hydrogen atom or a methyl group,
R₁₄ and R₁₅, independently of one another, represent a hydrogen or a methyl group, or R₁₄ and
R₁₅ form with the carbon atom carrying them a a cyclohexyl,
Rₕ and Rᵢ, independently of one another, represent a hydrogen or a linear or branched C₁-C₆alkyl group,
X₁ and X₂, independently of one another, represent a linear or branched
C₁-C₆alkylene group optionally substituted by one or two groups selected from the group consisting of trifluoromethyl, hydroxyl, a halogen, and C₁-C₆alkoxy,
X′₂represents a linear or branched C₁-C₆alkylene,
R′ₐ and R′_{b}, independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH: a C₁-C₆alkylene-SO₂O⁻; a C₁-C₆alkylene-COOH: a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH: a C₁-C₆alkylene-phenyl wherein the phenyl may be substituted by a hydroxyl or a C₁-C₆alkoxy group; and the group: 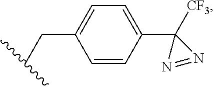 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
m=0, 1 or 2,
p=1, 2, 3 or 4,
B₃ and B₄, independently of one another, represents a C₃-C₅heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂. oxo and piperidinyl; or
(iii) the Bcl-xL inhibitor is represented by formula (IIB), (IIC), (IIB) or (IIIC): 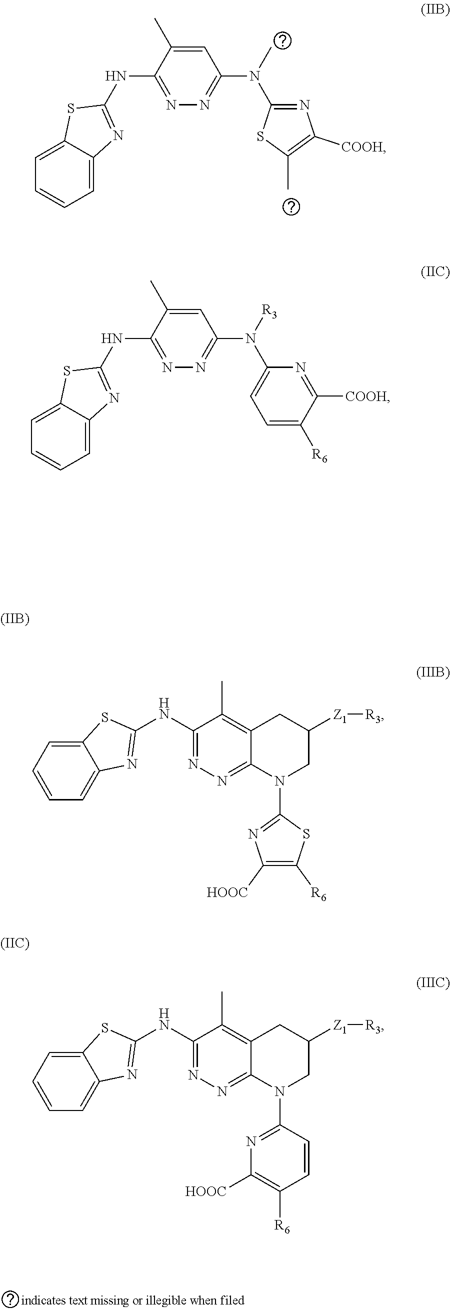
or an enantiomer, a diastereoisomer, and/or a pharmaceutically acceptable salt of any one of the foregoing, wherein:
for formula (IIB) or (IIC), R₃ represents a group selected from: hydrogen; linear or branched C₁-C₆alkyl; —X₁—NRₐR_{b}; —X₁—N⁺RₐR_{b}R_{c}; and —X₁—O—R_{c};
for formula (IIIB) or (IIIC), Zi represents a bond, and R₃ represents hydrogen; or Zi represents -O—, and R₃ represents —X₁—NRₐR_{b},
Rₐ and R_{b}, independently of one another, represent a group selected from: hydrogen; linear or branched C₁-C₆alkyl optionally substituted by one or two hydroxyl groups; and C₁-C₆alkylene
SO₂O⁻,
R_{c} represents a hydrogen or a linear or branched C₁-C₆alkyl group
R₆ represents —X₂—O—R₇ or an heteroarylene-R₇ group optionally substituted by a linear or branched C₁-C₆alkyl group,
R₇ represents a group selected from: 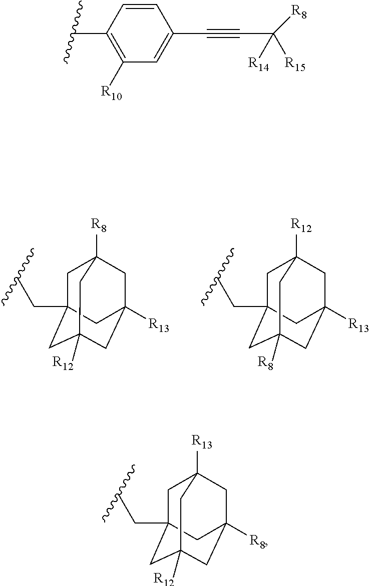
R₈ represents a group selected from: —NR′ₐR′_{b}; —O—X′₂—NR′ₐR′_{b}; and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂, independently of one another, represent a linear or branched
C₁-C₆alkylene group optionally substituted by one or two groups selected from trifluoromethyl, hydroxyl, halogen, C₁-C₆alkoxy,
X′₂represents a linear or branched C₁-C₆alkylene,
R′ₐ and R′_{b} independently of one another, represent a group selected from: hydrogen; linear or branched C₁-C₆alkyl optionally substituted by one or two hydroxyl or C₁-C₆alkoxy groups; C₁-C₆alkylene-NR′R′ₑ;
or R′ₐ and R′_{b} form with the nitrogen atom carrying them a cycle B₃, R′_{d}, R′ₑ, independently of one another, represents a hydrogen or a linear or branched C₁-C₆alkyl group,
B₃ represents a C₃-C₈heterocycloalkyl group, which group can: (a) be a mono- or bi-cyclic group, wherein bicyclic group includes fused, bridged or spiro ring system, (b) can contain, in addition to the nitrogen atom, one or two hetero atoms selected independently from oxygen and nitrogen, (c) be substituted by one or two groups selected from: fluorine, bromine, chlorine, linear or branched C₁-C₆alkyl, hydroxyl, and oxo.

104. (canceled)

105. The antibody-drug conjugate of claim 103, wherein;
(i) G is selected from the group consisting of: —C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN;
(ii) R₇ represents a group selected from the group consisting of: a linear or branched C₁-C₆alkyl group; a (C₃-C₆)cycloalkylene-R₈; 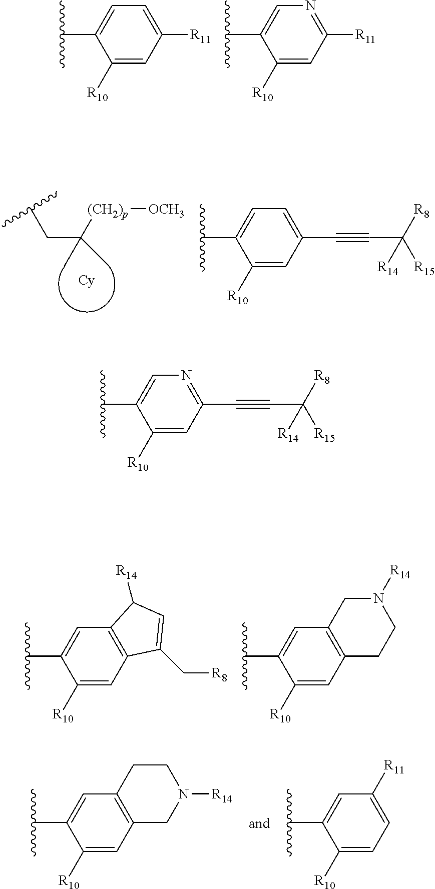
wherein Cy represents a C₃-C₈cycloalkyl
(iii) R₇ represents a group selected from the group consisting of 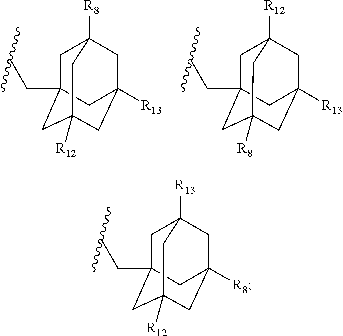
(iv) R₇ represents the following group 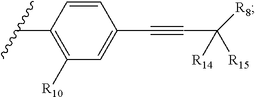
(v) R₇ represents a group selected from: 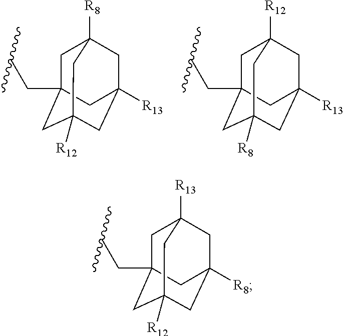
(vi) R₈ represents a group selected from: 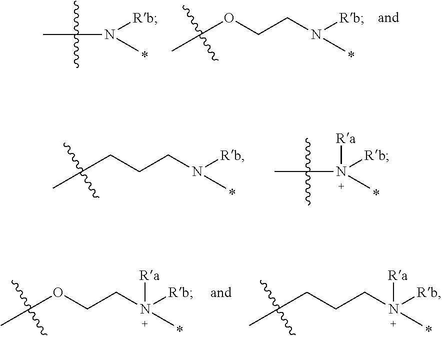
wherein [symbol] represents a bond to the linker; and/or
(vii) B₃ represents a C₃-C₈heterocycloalkyl group selected from a pyrrolidinyl group, a piperidinyl group, a piperazinyl group, a morpholinyl group, an azepanyl group, and a 4,4-difluoropiperidin-1-yl group.

106-112. (canceled)

113. The antibody-drug conjugate of claim 103, wherein the Bcl-xL inhibitor is represented by any one of the following: 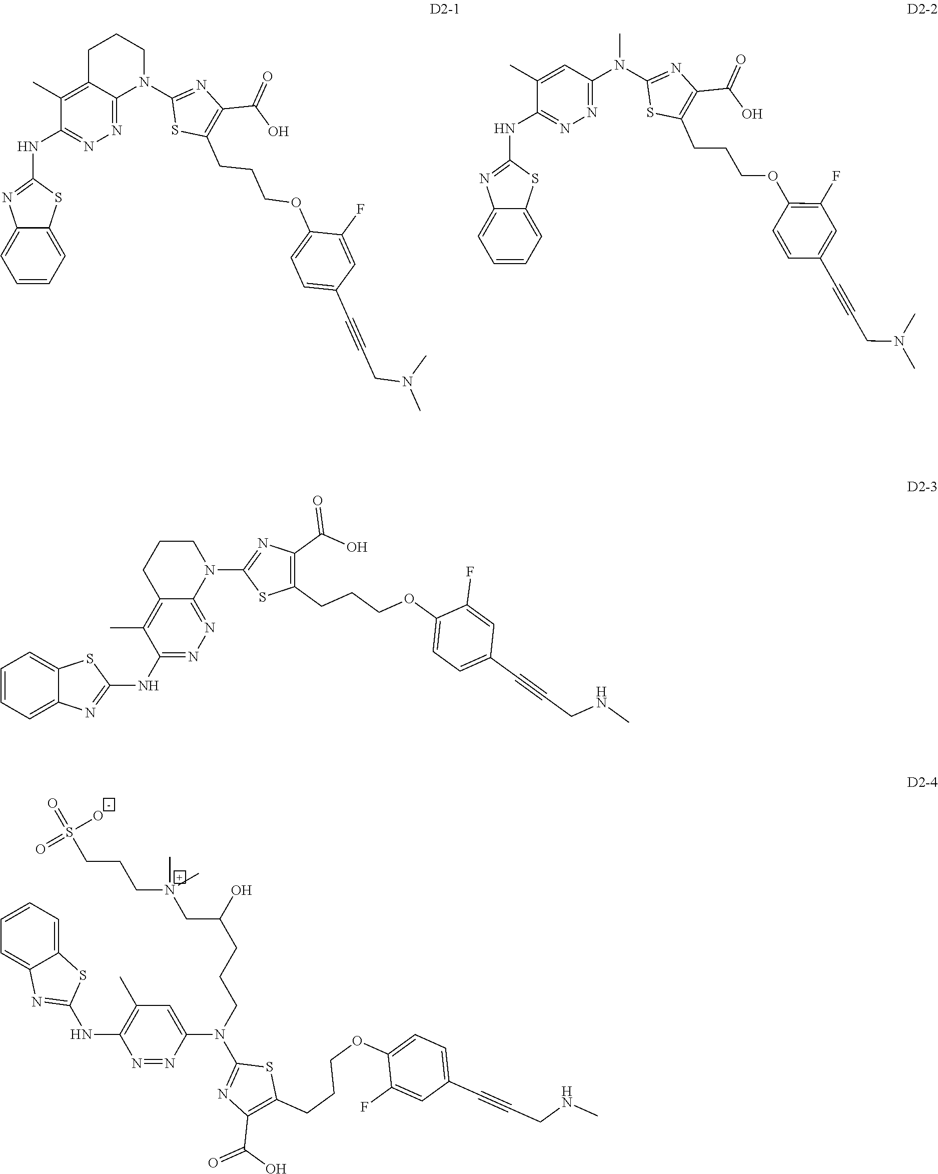 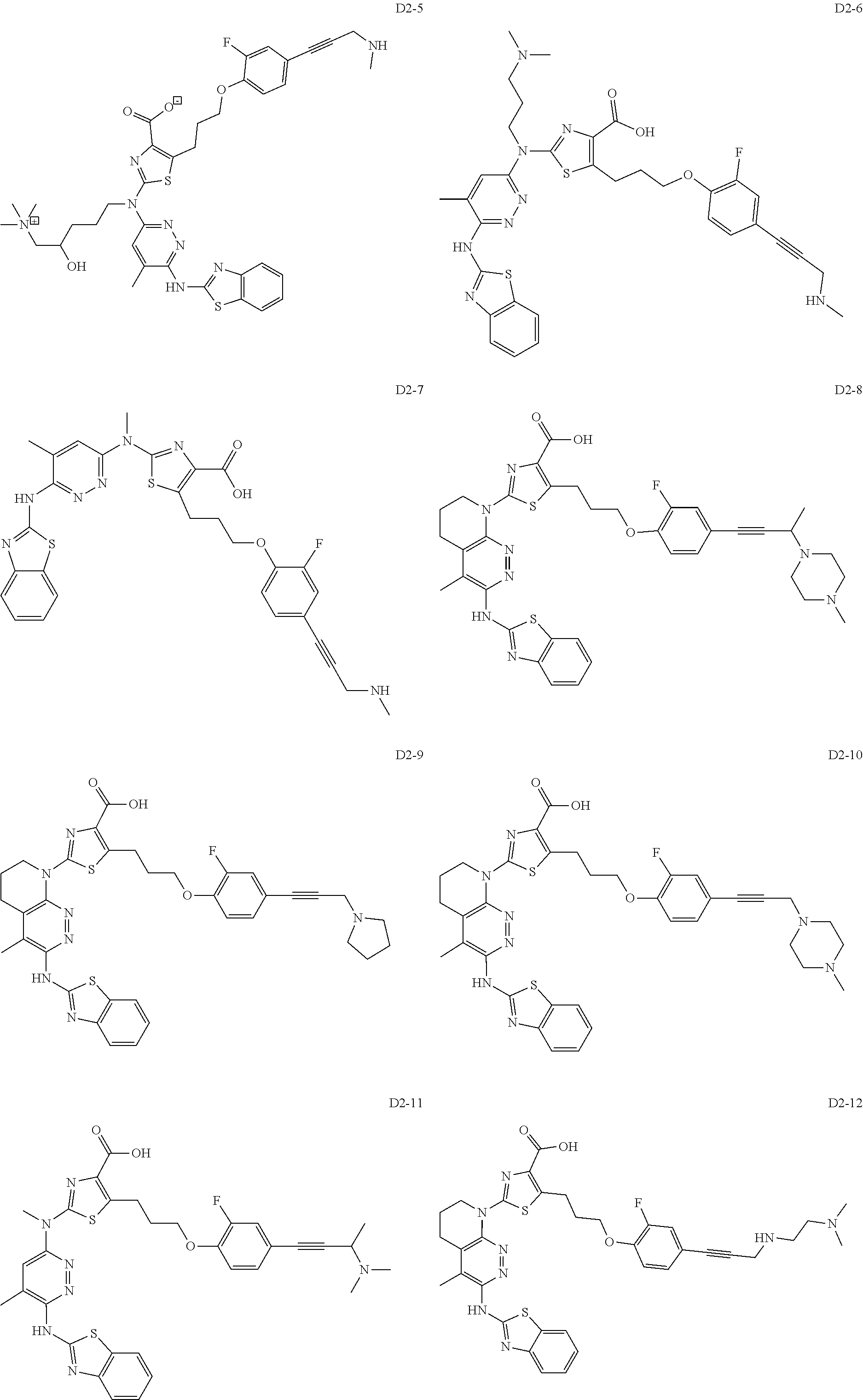 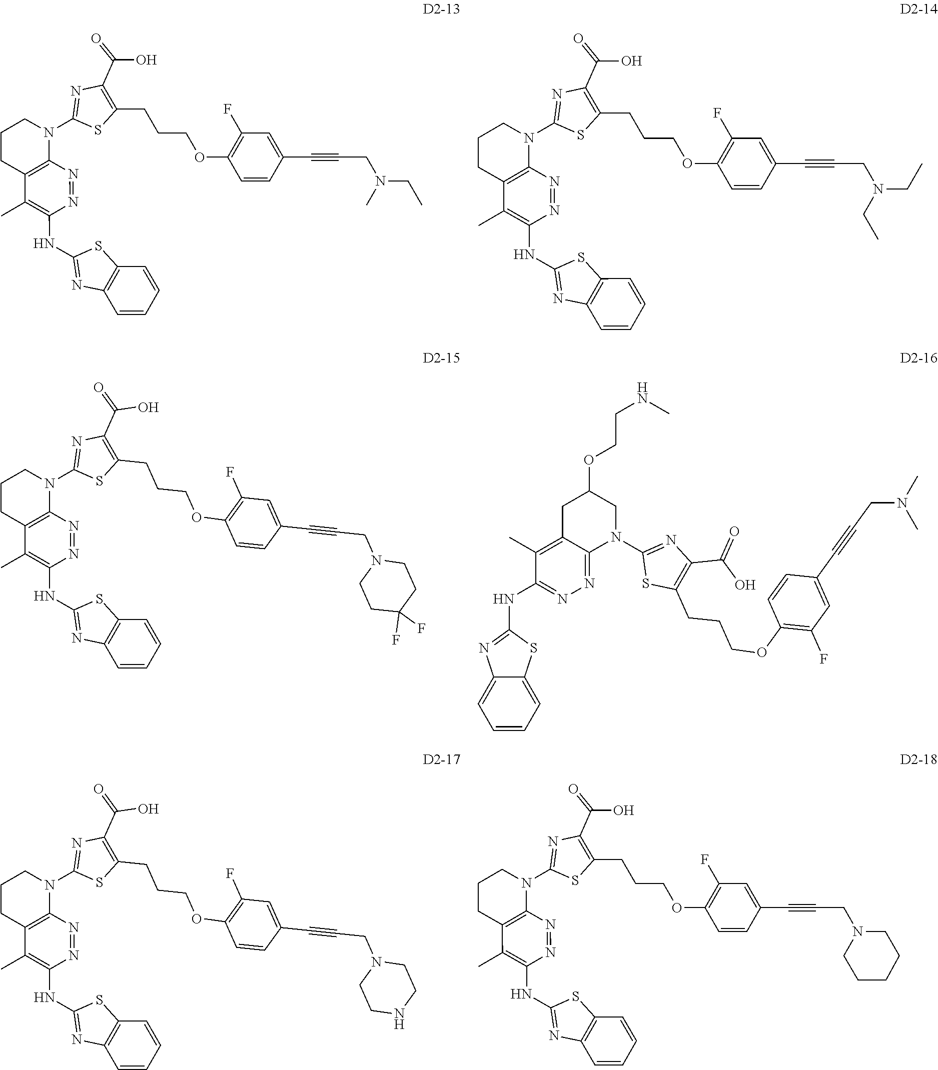 [SMILES: CCN(C)CC#Cc1ccc(OCCCc2sc(N3CCCc4c3nnc(Nc3nc5ccccc5s3)c4C)nc2C(=O)O)c(F)c1 ; CCN(CC)CC#Cc1ccc(OCCCc2sc(N3CCCc4c3nnc(Nc3nc5ccccc5s3)c4C)nc2C(=O)O)c(F)c1 ; Cc1c(Nc2nc3ccccc3s2)nnc2c1CCCN2c1nc(C(=O)O)c(CCCOc2ccc(C#CCN3CCC(F)(F)CC3)cc2F)s1 ; CNCCOC1Cc2c(nnc(Nc3nc4ccccc4s3)c2C)N(c2nc(C(=O)O)c(CCCOc3ccc(C#CCN(C)C)cc3F)s2)C1 ; Cc1c(Nc2nc3ccccc3s2)nnc2c1CCCN2c1nc(C(=O)O)c(CCCOc2ccc(C#CCN3CCNCC3)cc2F)s1 ; Cc1c(Nc2nc3ccccc3s2)nnc2c1CCCN2c1nc(C(=O)O)c(CCCOc2ccc(C#CCN3CCCCC3)cc2F)s1] 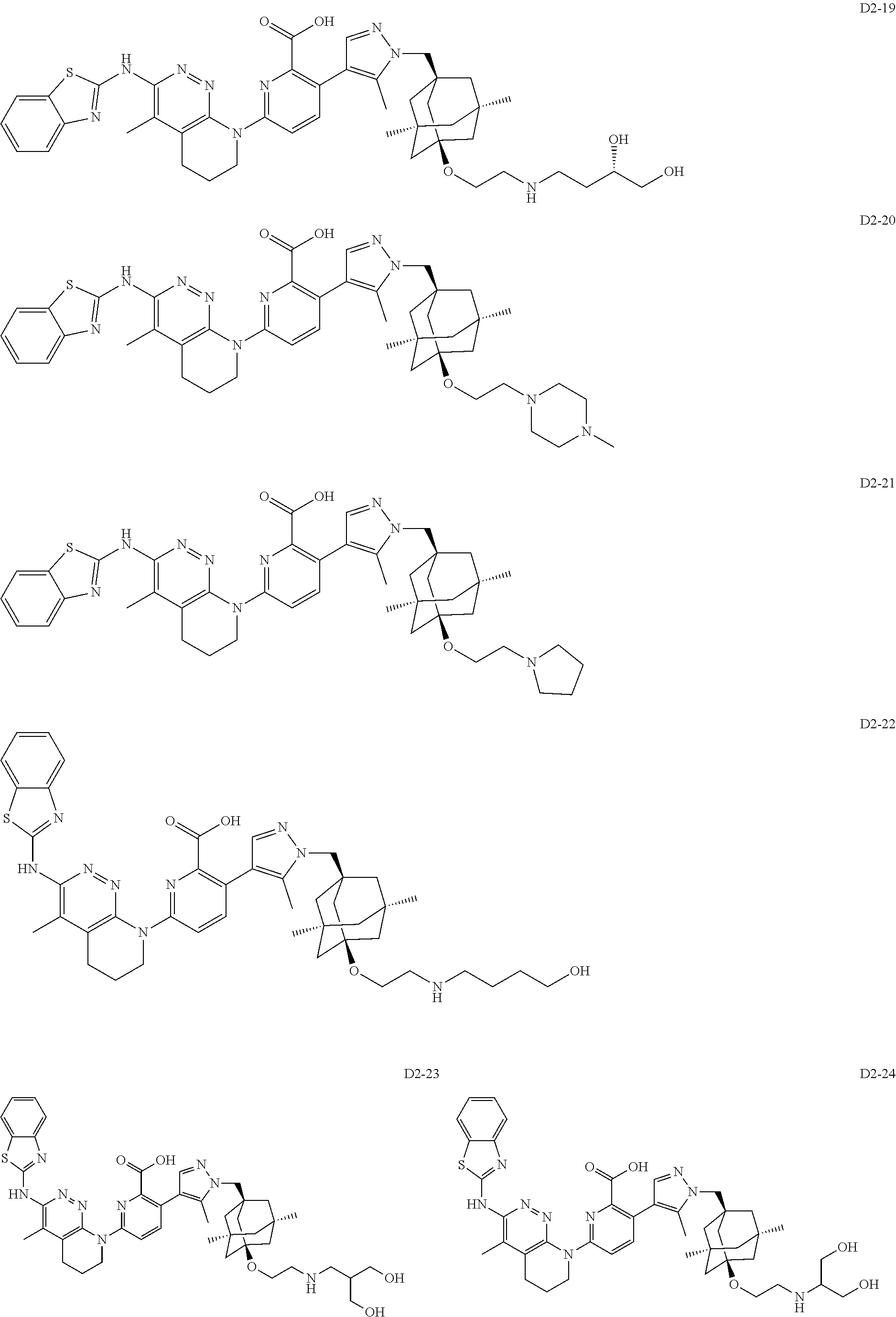 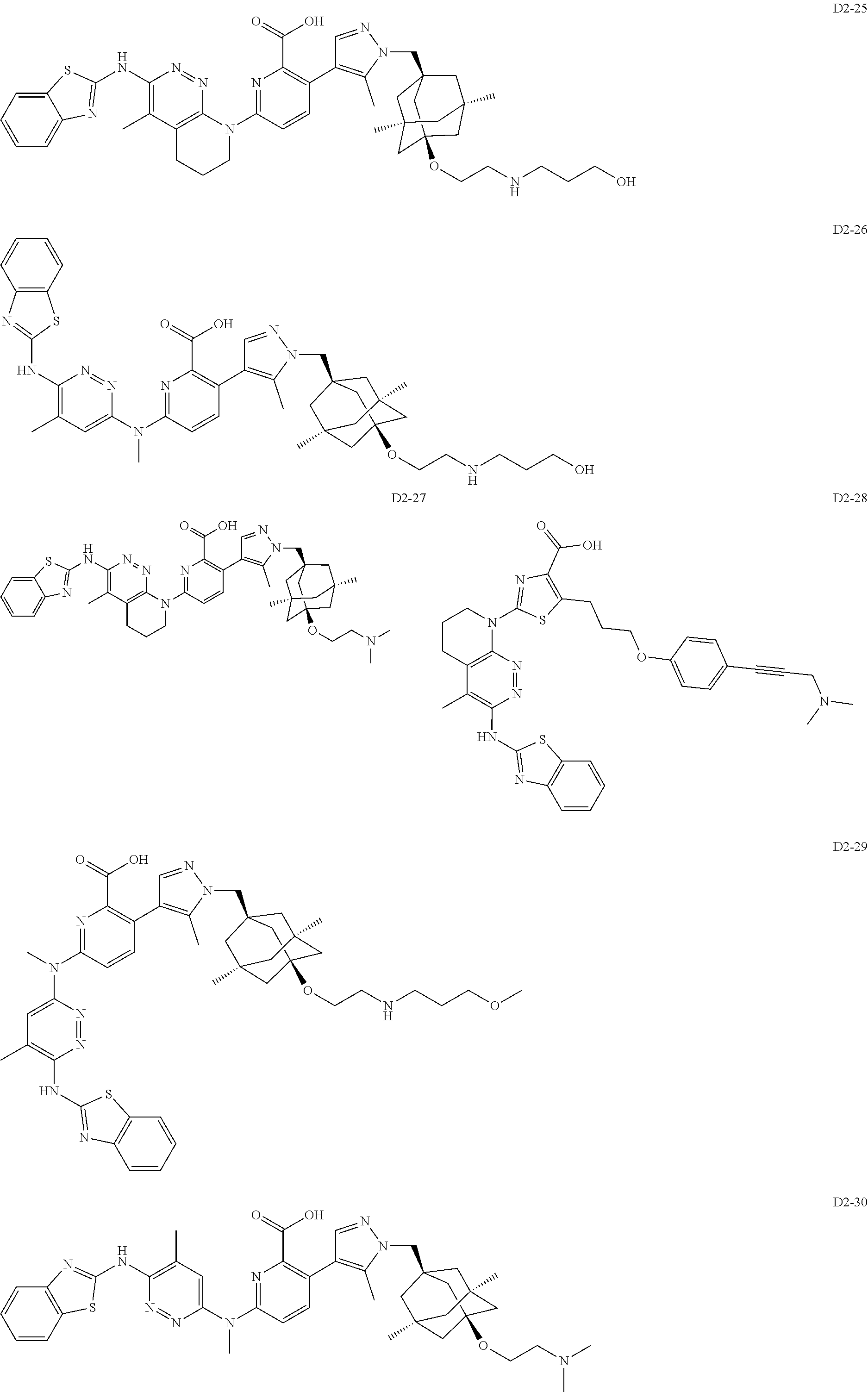 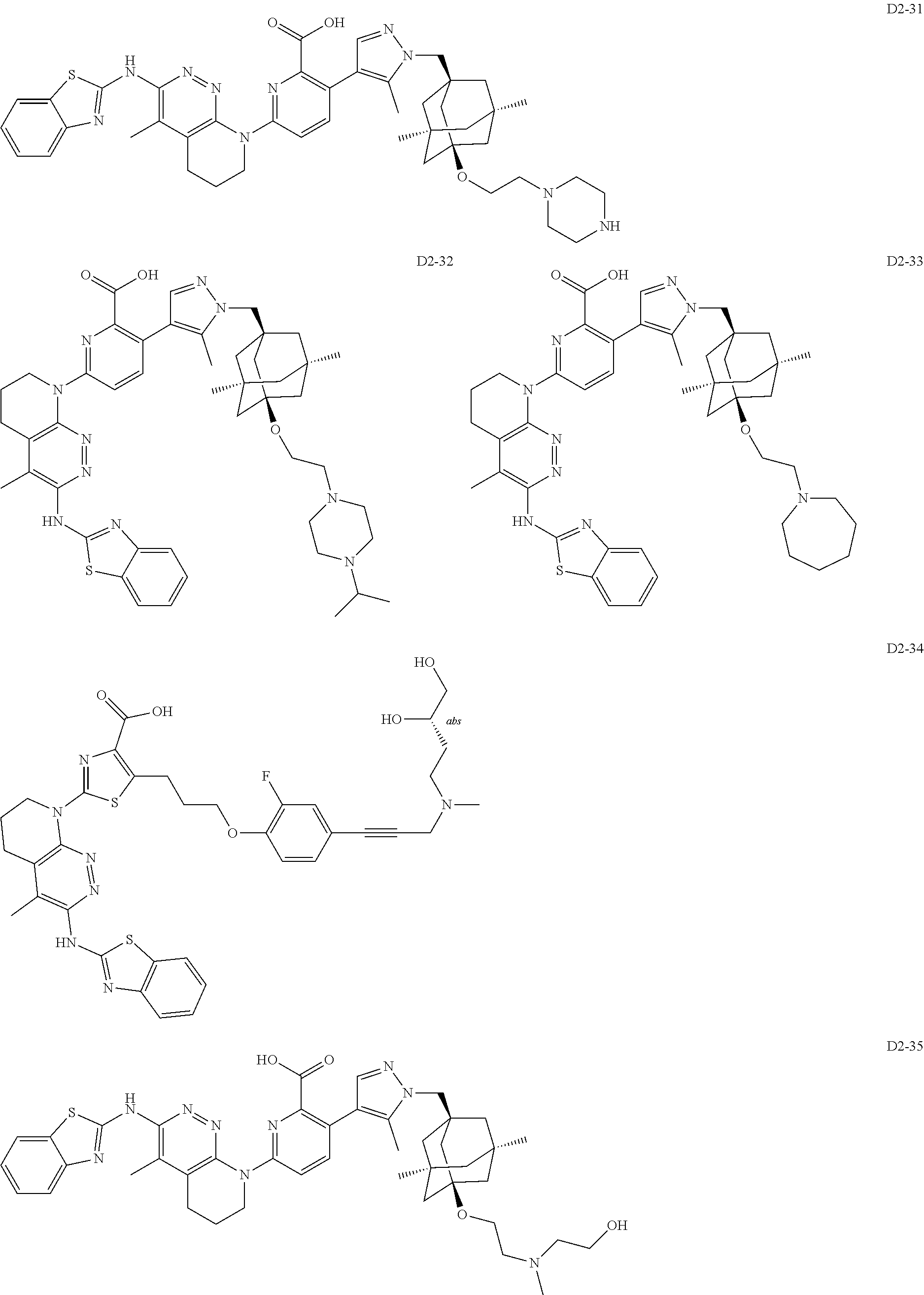 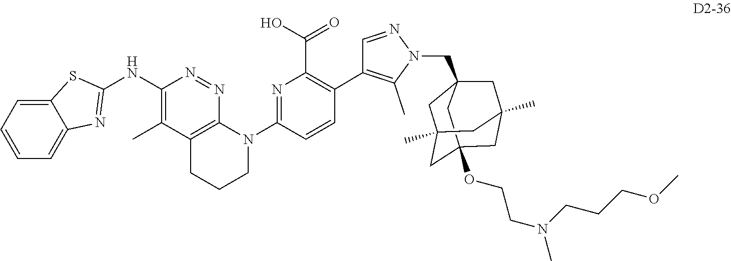 [SMILES: COCCCN(C)CCO[C@]12C[C@@]3(C)C[C@](C)(C[C@](Cn4ncc(-c5ccc(N6CCCc7c6nnc(Nc6nc8ccccc8s6)c7C)nc5C(=O)O)c4C)(C3)C1)C2] 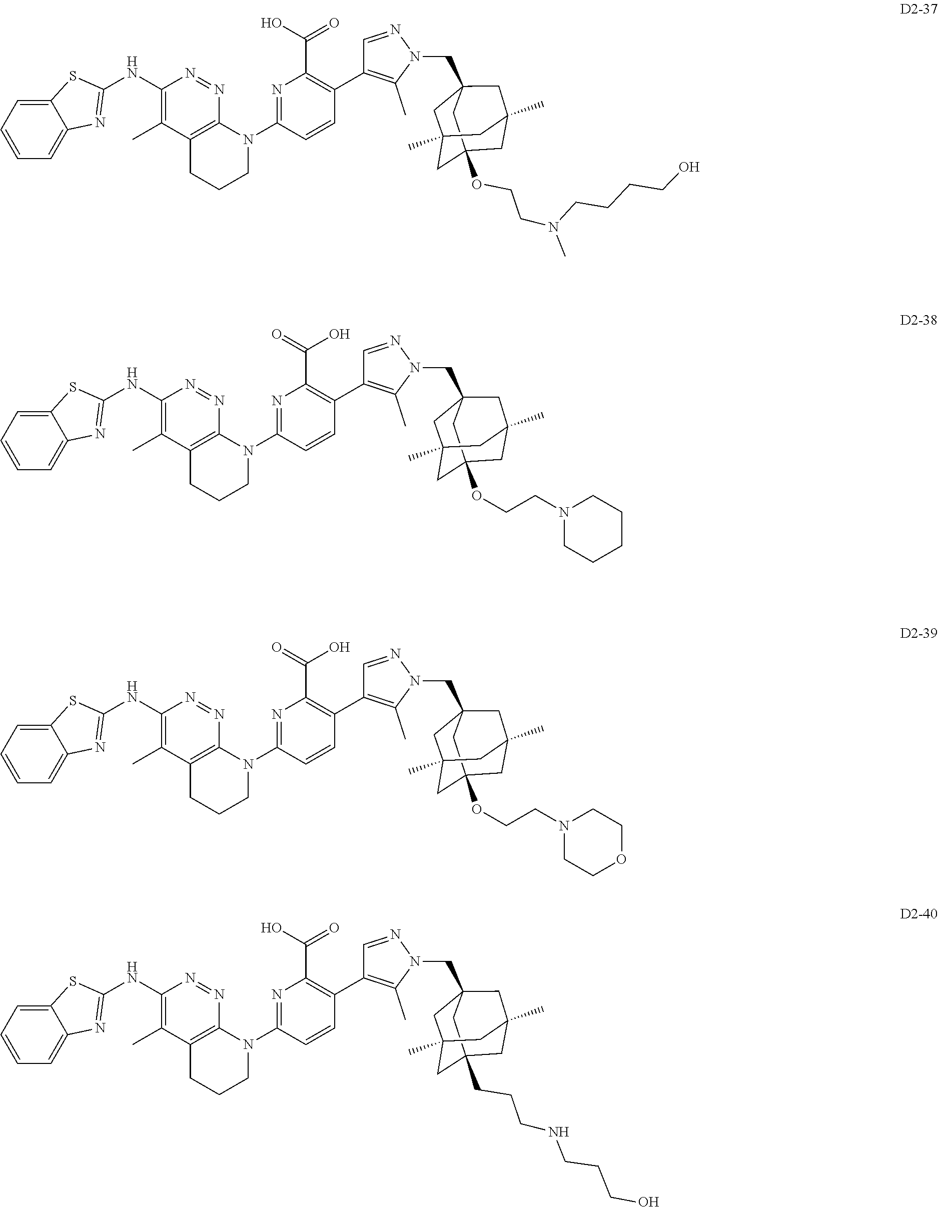 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)CCCCO)(C5)C4)c2C)c(C(=O)O)n1 ; Cc1c(Nc2nc3ccccc3s2)nnc2c1CCCN2c1ccc(-c2cnn(C[C@]34C[C@]5(C)C[C@](C)(C3)C[C@@](OCCN3CCCCC3)(C5)C4)c2C)c(C(=O)O)n1 ; Cc1c(Nc2nc3ccccc3s2)nnc2c1CCCN2c1ccc(-c2cnn(C[C@]34C[C@]5(C)C[C@](C)(C3)C[C@@](OCCN3CCOCC3)(C5)C4)c2C)c(C(=O)O)n1 ; Cc1c(Nc2nc3ccccc3s2)nnc2c1CCCN2c1ccc(-c2cnn(C[C@]34C[C@@]5(C)C[C@](C)(C[C@](CCCNCCCO)(C5)C3)C4)c2C)c(C(=O)O)n1] 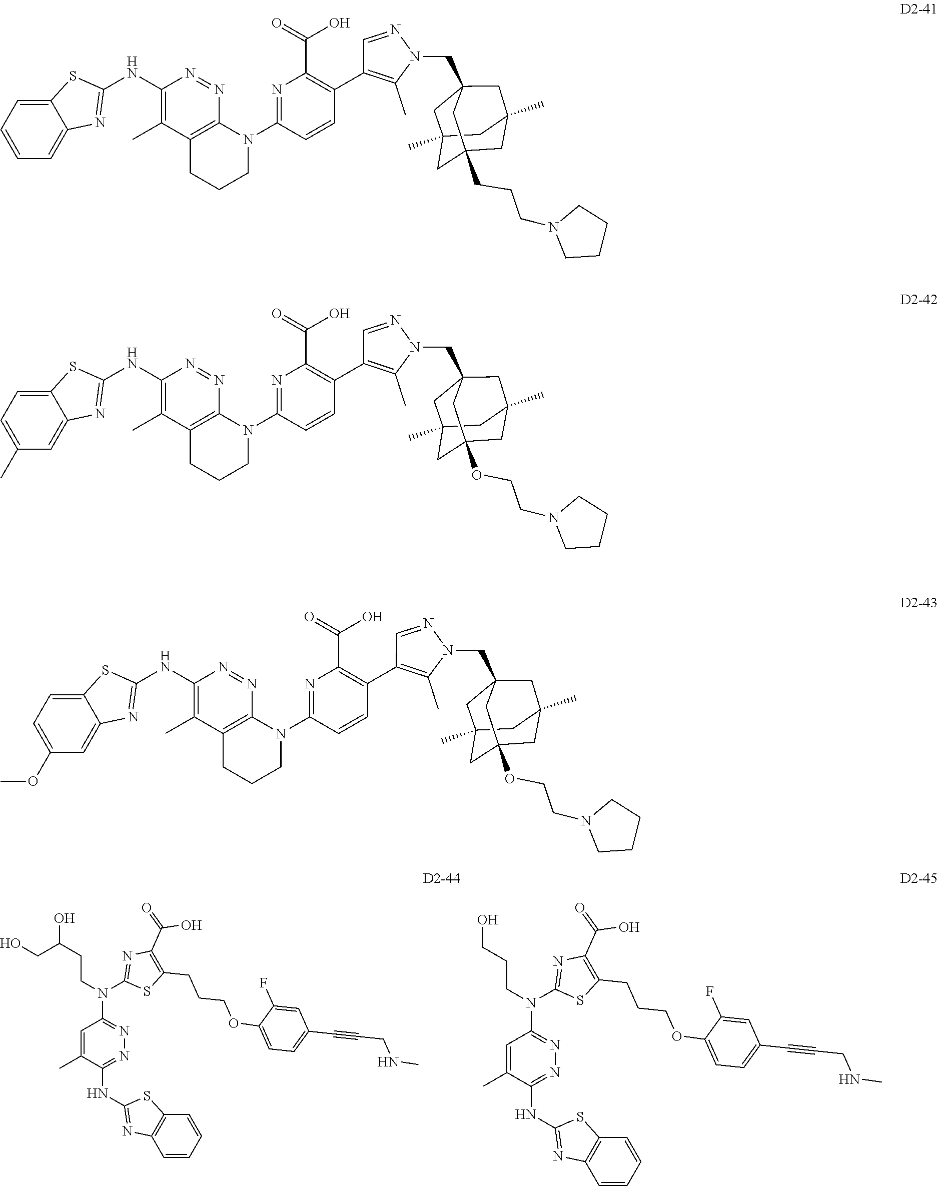 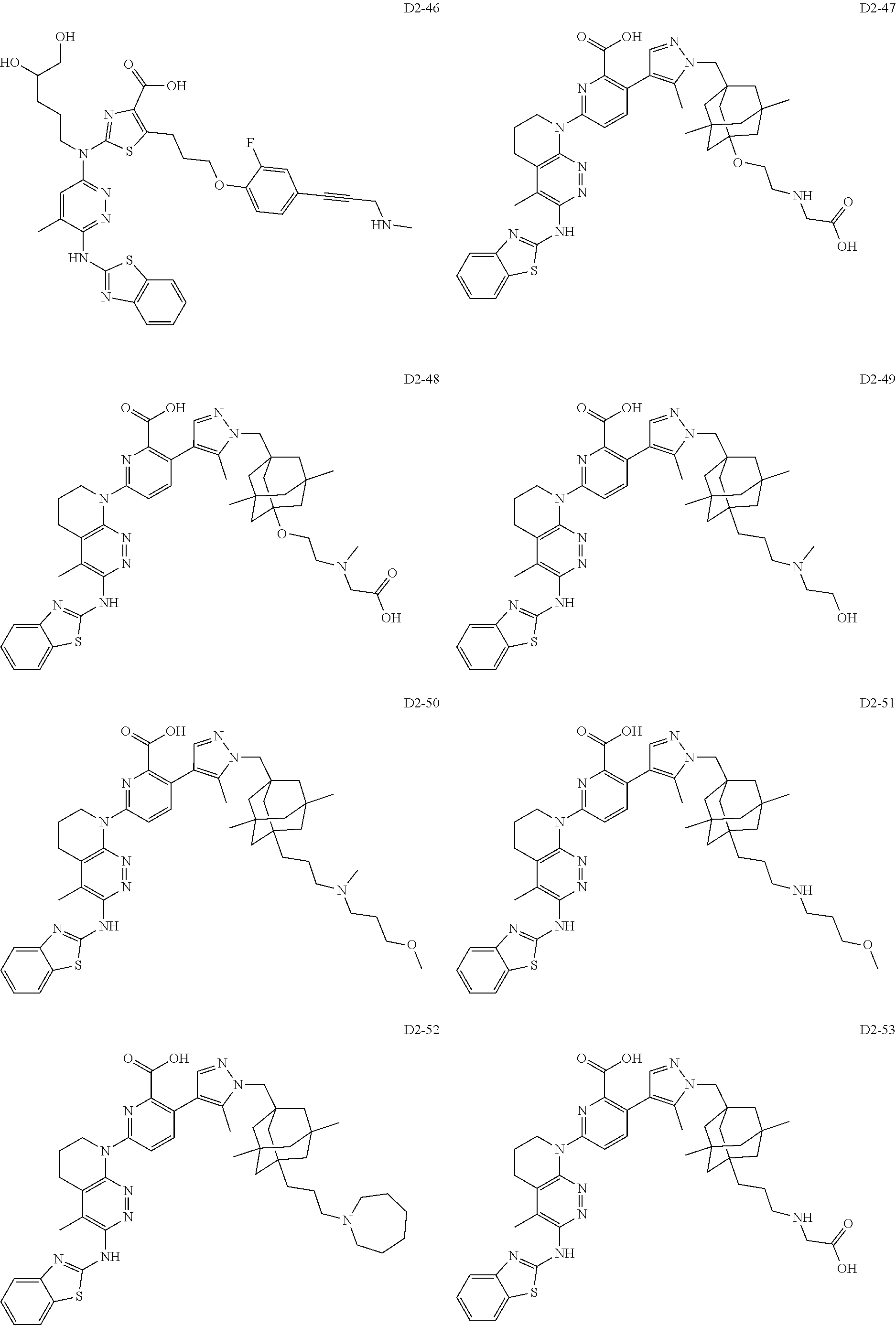 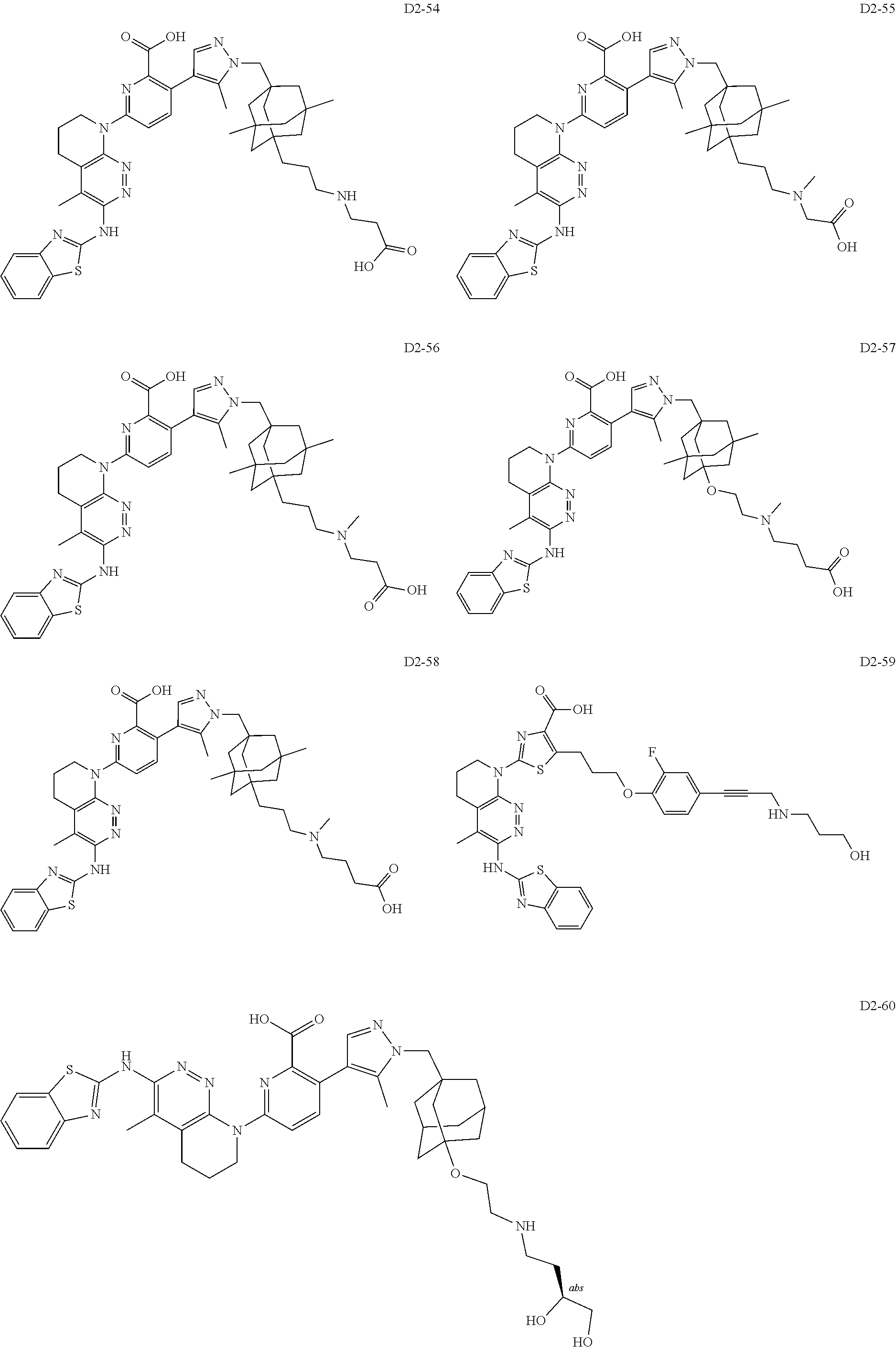 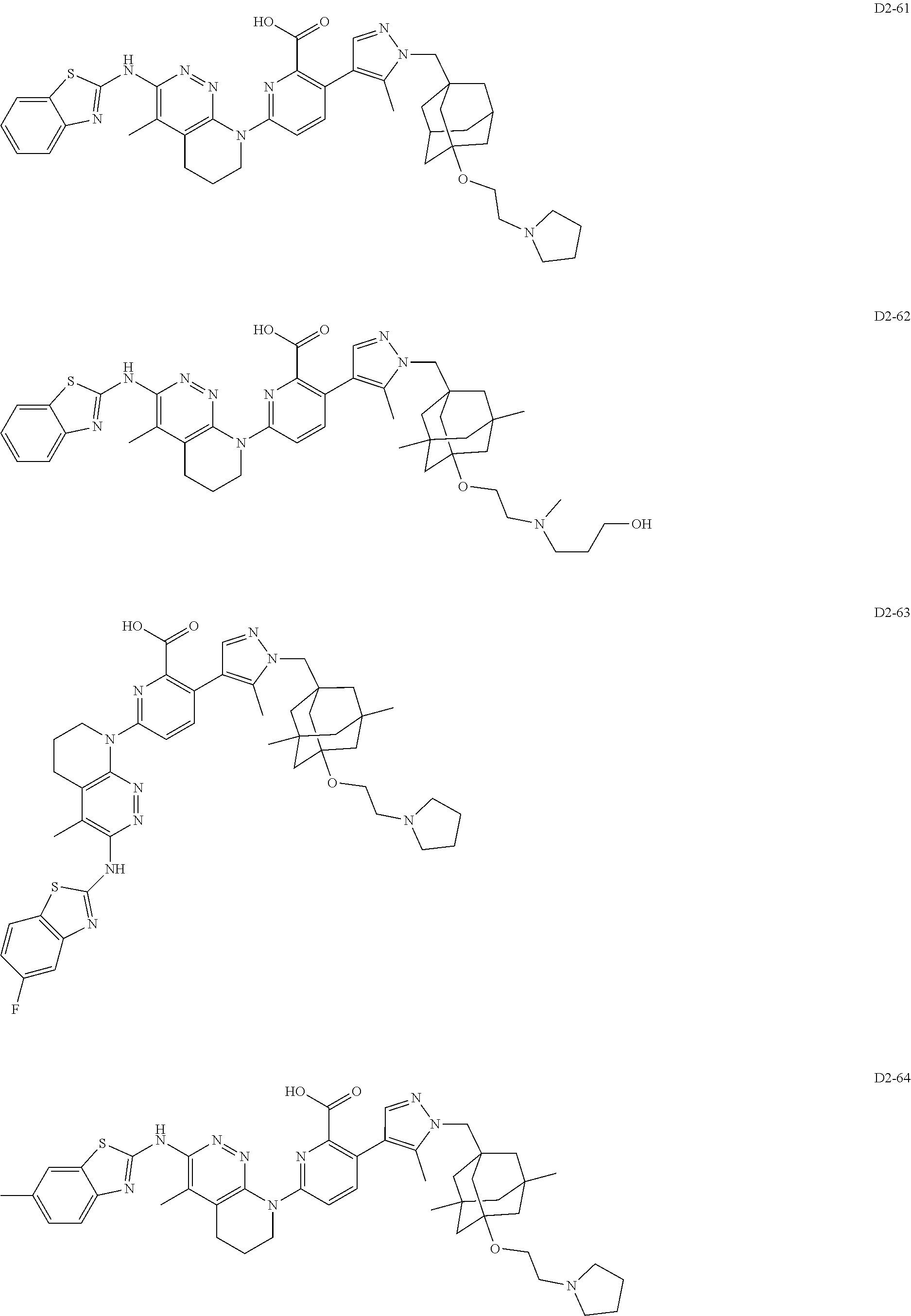 [SMILES: Cc1c(Nc2nc3ccccc3s2)nnc2c1CCCN2c1ccc(-c2cnn(CC34CC5CC(C3)CC(OCCN3CCCC3)(C5)C4)c2C)c(C(=O)O)n1 ; Cc1c(Nc2nc3ccccc3s2)nnc2c1CCCN2c1ccc(-c2cnn(CC34CC5(C)CC(C)(C3)CC(OCCN(C)CCCO)(C5)C4)c2C)c(C(=O)O)n1 ; Cc1c(Nc2nc3cc(F)ccc3s2)nnc2c1CCCN2c1ccc(-c2cnn(CC34CC5(C)CC(C)(C3)CC(OCCN3CCCC3)(C5)C4)c2C)c(C(=O)O)n1 ; Cc1ccc2nc(Nc3nnc4c(c3C)CCCN4c3ccc(-c4cnn(CC56CC7(C)CC(C)(C5)CC(OCCN5CCCC5)(C7)C6)c4C)c(C(=O)O)n3)sc2c1] 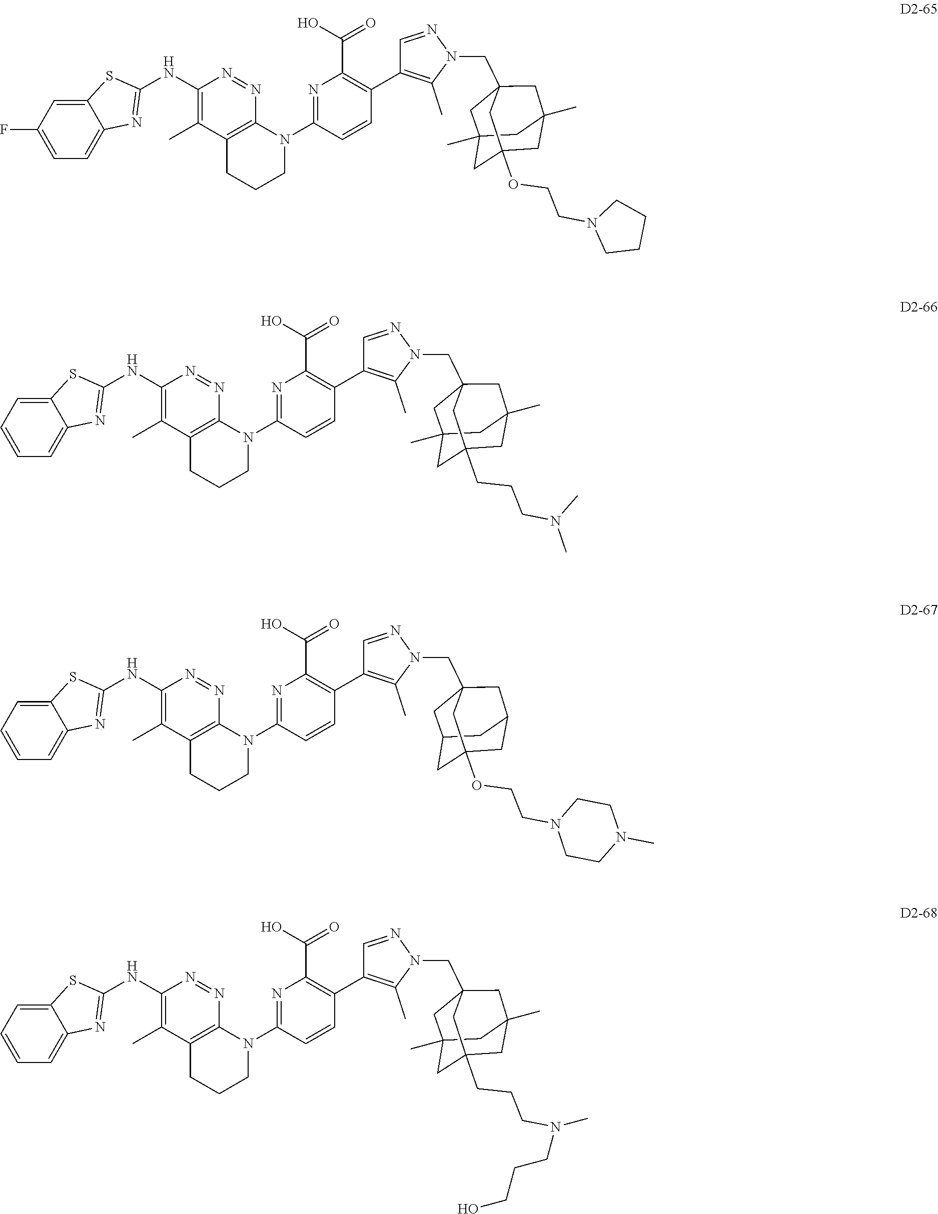 [SMILES: Cc1c(Nc2nc3ccc(F)cc3s2)nnc2c1CCCN2c1ccc(-c2cnn(CC34CC5(C)CC(C)(C3)CC(OCCN3CCCC3)(C5)C4)c2C)c(C(=O)O)n1 ; Cc1c(Nc2nc3ccccc3s2)nnc2c1CCCN2c1ccc(-c2cnn(CC34CC5(C)CC(C)(CC(CCCN(C)C)(C5)C3)C4)c2C)c(C(=O)O)n1 ; Cc1c(Nc2nc3ccccc3s2)nnc2c1CCCN2c1ccc(-c2cnn(CC34CC5CC(C3)CC(OCCN3CCN(C)CC3)(C5)C4)c2C)c(C(=O)O)n1 ; Cc1c(Nc2nc3ccccc3s2)nnc2c1CCCN2c1ccc(-c2cnn(CC34CC5(C)CC(C)(CC(CCCN(C)CCCO)(C5)C3)C4)c2C)c(C(=O)O)n1] 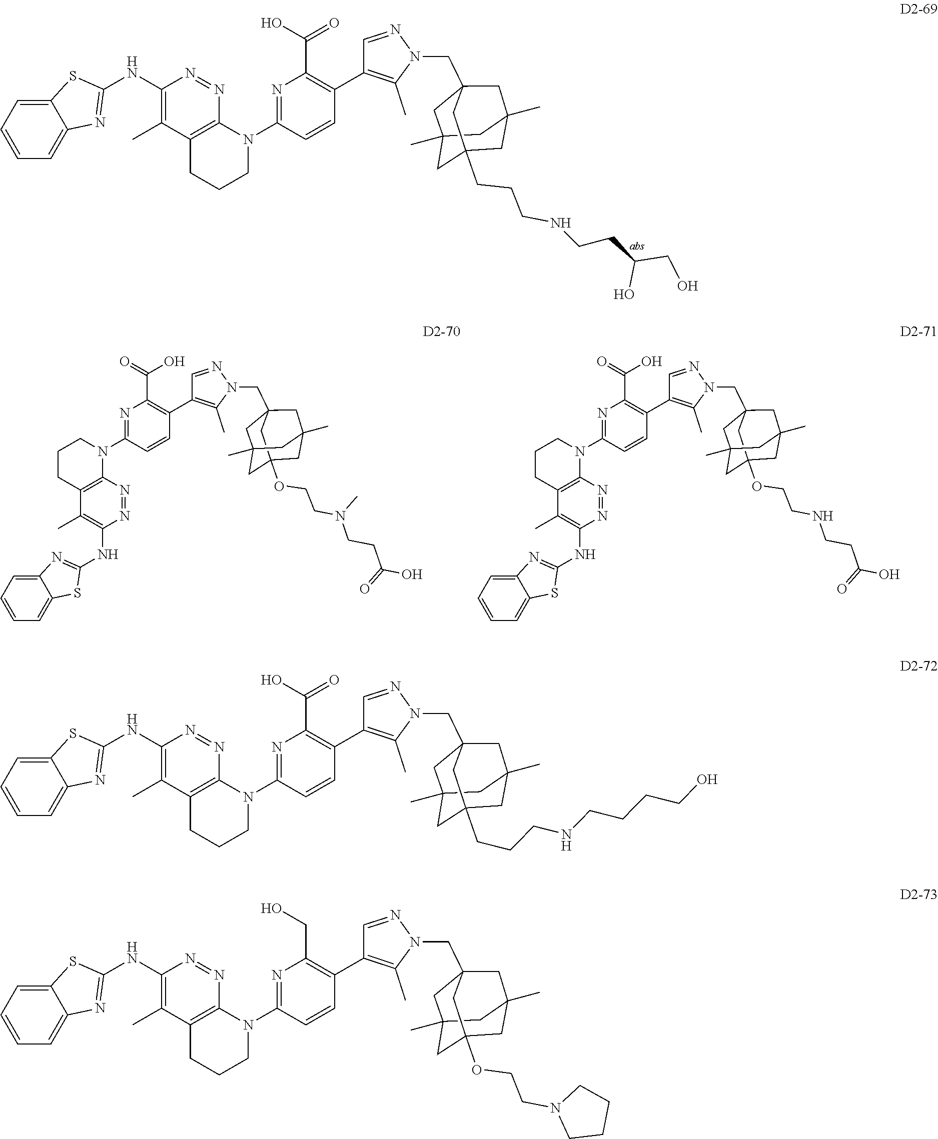 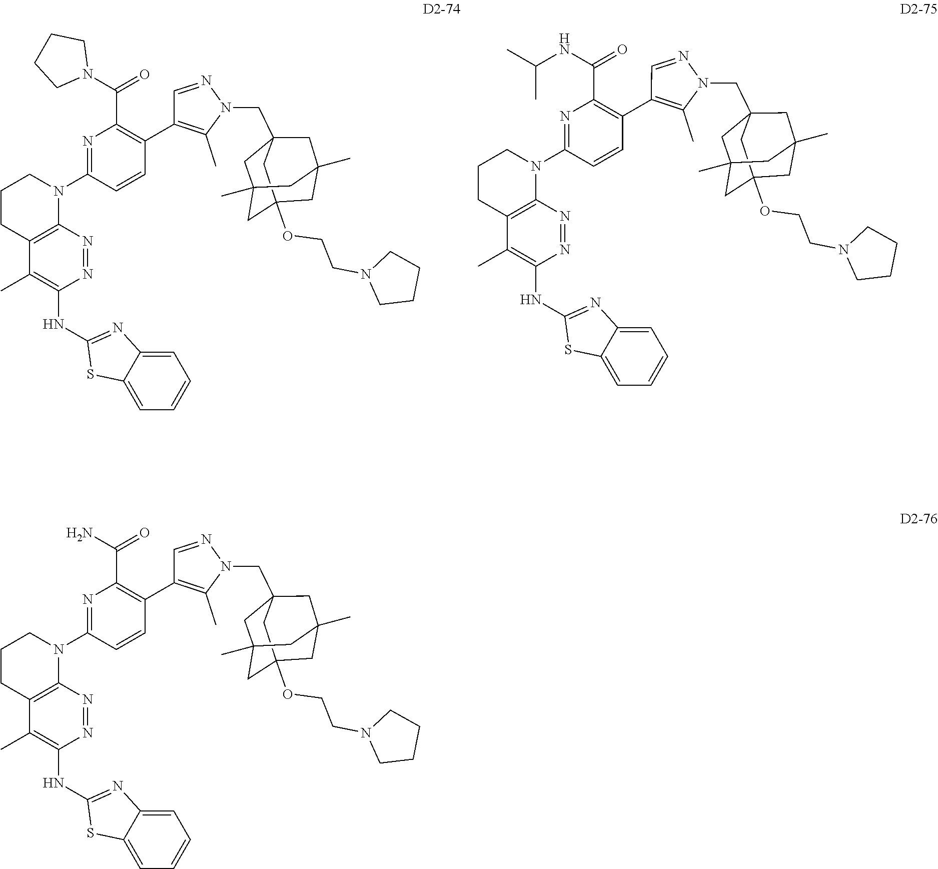 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)N2CCCC2)n1 ; Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)NC(C)C)n1 ; Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(N)=O)n1]
or an enantiomer, a diastereoisomer, and/or a pharmaceutically acceptable salt of any one of the foregoing.

114. The antibody-drug conjugate of claim 1, or pharmaceutically acceptable salt thereof, wherein the Bcl-2 inhibitor is represented by Formula (IV) or Formula (V): 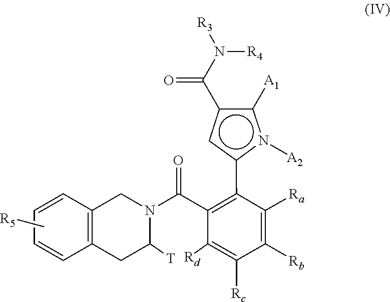
or an enantiomer, a diastereoisomer, and/or a pharmaceutically acceptable salt of any one of the foregoing, wherein:
A₁ represents a hydrogen or halogen atom, a linear or branched (C₁-C₆)polyhaloalkyl group, a linear or branched (C₁-C₆)alkyl group or a cycloalkyl group,
A₂ represents a linear or branched (C₁-C₆)alkyl group optionally substituted by a group selected from halogen, hydroxy, linear or branched (C₁-C₆)alkoxy, NR′R″ and morpholine, or A₂ represents a linear or branched (C₁-C₆)polyhaloalkyl group or a cyclopropyl group, it being understood that R′and R″, each independently of the other, represent a hydrogen atom or a linear or branched (C₁-C₆)alkyl group,
T represents a hydrogen atom, a linear or branched (C₁-C₆)alkyl group optionally substituted by from one to three halogen atoms, a group (C₁-C₄)alkyl-NR₁R², or a group (C₁-C₄)alkyl-OR₆,
R₁ and R₂, each independently of the other, represent a hydrogen atom or a linear or branched (C₁-C₆)alkyl group,
or R₁ and R₂ form with the nitrogen atom carrying them a heterocycloalkyl,
R₃ represents an aryl or heteroaryl group, it being understood that one or more carbon atoms of the preceding groups, or of their possible substituents, may be deuterated,
R₄ represents a phenyl group, a 4-hydroxyphenyl group, a 3-fluoro-4-hydroxyphenyl group, a 2-hydroxypyrimidine group or a 3-hydroxypyridine group, it being understood that one or more carbon atoms of the preceding groups, or of their possible substituents, may be deuterated,
R₅ represents a hydrogen or halogen atom, a linear or branched (C₁-C₆)alkyl group, or a linear or branched (C₁-C₆)alkoxy group,
R₆ represents a hydrogen atom or a linear or branched (C₁-C₆)alkyl group,
Rₐ and R_{d} each represent a hydrogen atom and (R_{b},R_{c}) form together with the carbon atoms carrying them a 1,3-dioxolane group or a 1,4-dioxane group, or Rₐ, R_{c} and R_{d} each represent a hydrogen atom and R_{b} represents a hydrogen or halogen atom or a methoxy group,
or Rₐ and R_{d} each represent a hydrogen atom, R_{b} represents a hydrogen or halogen atom and R_{c} represents a hydroxy or methoxy group, or: Rₐ and R_{d} each represent a hydrogen atom, R_{b} represents a hydroxy or methoxy group and R_{c} represents a halogen atom, or 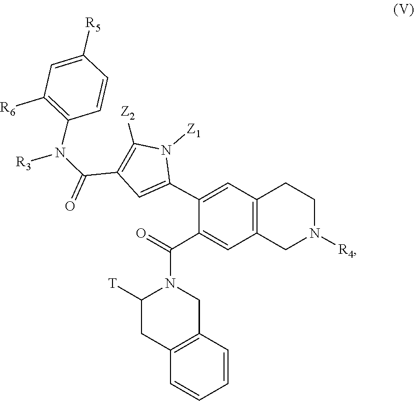
or an enantiomer, a diastereoisomer, and/or a pharmaceutically acceptable salt of any one of the foregoing, wherein:
Z₁ and Z₂ represent both a methyl group or they form together with the atoms carrying them a fused piperidine group,
T represents a hydrogen atom, a linear or branched (C₁-C₆)alkyl group optionally substituted by one to three halogen atoms, a (C₁-C₄)alkylene-NR₁R² group, a (C₁-C₄)alkylene-ORᵢ group,
R₁ and R₂, independently of one another, represent a hydrogen atom or a linear or branched (C₁-C₆)alkyl group,
or Rᵢ and R₂ form with the nitrogen atom carrying them a heterocycloalkyl group, which heterocycloalkyl is optionally substituted by one to three groups selected from: (C₁-C₆)alkyl group and halogen atom,
R₃ represents a group selected from: 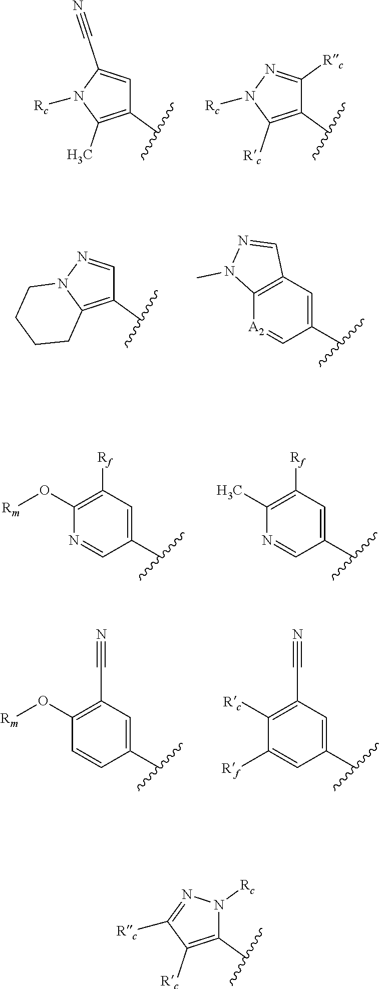
R₄ represents a group selected from: 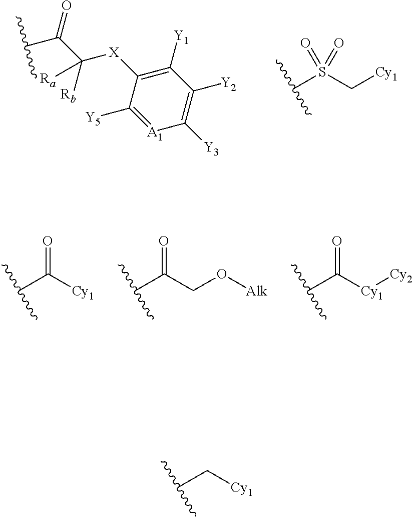
R₅ represents a hydrogen atom, a halogen atom or a hydroxy group,
R₆ represents a hydrogen, a linear or branched (C₁-C₆)alkyl group, or a halogen atom, Alk represents a linear or branched (C₁-C₆)alkyl group,
A₁ represents a C—Y₄ or a nitrogen atom,
A₂ represents a C—H or a nitrogen atom,
Cy₁ represents a phenyl, a heteroaryl, a cycloalkyl or a heterocycloalkyl group, wherein the phenyl, the heteroaryl, the cycloalkyl and the heterocycloalkyl groups are optionally substituted by one to three groups selected from: linear or branched (C₁-C₆)alkyl group optionally substituted by 1 to 3 halogen atoms, hydroxy group, cycloalkyl group, and halogen atom and the heterocycloalkyl group is optionally further substituted by an oxo group,
Cy₂ represent a phenyl or a heteroaryl group, wherein the phenyl and the heteroaryl groups are optionally substituted by one to three groups selected from: linear or branched (C₁-C₆)alkyl group optionally substituted by 1 to 3 halogen atoms, hydroxy group, and halogen atom
X represents a bond, —O—, —S— or NRₖ,
Y₁ and Y₅, independently of one another, represent a group selected from: hydrogen atom, halogen atom, cyano, linear or branched (C₁-C₆)alkyl group, and linear or branched (C₁-C₆)alkoxy group,
Y₂ and Y₄, independently of one another, represent a group selected from: hydrogen atom, halogen atom, linear or branched (C₁-C₆)alkyl group, linear or branched (C₁-C₆)alkoxy group, and heterocycloalkyl group optionally substituted by a linear or branched (C₁-C₆)alkyl group,
Y₃ represents a group selected from: hydrogen atom, halogen atom, linear or branched (C₁-C₆)alkyl, linear or branched (C₁-C₆)alkynyl, —(C₁-C₄)alkylene-ORI, linear or branched (C₁-C₆)alkoxy group, —O-phenyl, —S-phenyl, —O—(C₁-C₄)alkylene-Cy₃, —O—(C₁-C₄)alkylene-Cy₄, —O—Cy₃, —O—(C₁-C₄)alkylene-NR_{g}Rₕ, —(C₁-C₄)alkylene-Cy₃, —(C₁-C₄)alkylene-Cy₄, Cy₃, Cy₄, and: 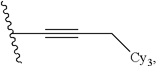
wherein the alkylene moiety of the preceding groups may be linear or branched,
Cy₃ represents a heterocycloalkyl optionally substituted by one to three groups selected from: linear or branched (C₁-C₆)alkyl group optionally substituted by 1 to 3 halogen atoms, hydroxy group, cycloalkyl group, heterocycloalkyl group, and halogen atom,
Cy₄ represents a cycloalkyl optionally substituted by one to three groups selected from: linear or branched (C₁-C₆)alkyl group optionally substituted by 1 to 3 halogen atoms, hydroxy group, cycloalkyl group, heterocycloalkyl group, and halogen atom, Rₐ and R_{b}, independently of one another, represent a hydrogen atom or a halogen atom, R_{c} represents a group selected from: hydrogen, linear or branched (C₁-C₆)alkyl group optionally substituted by 1 to 3 halogen atoms, (C₁-C₆)alkylene-NR_{d}Rₑ, (C₁-C₆)alkylene-ORⱼ, cycloalkyl, heterocycloalkyl, and (C₁-C₆)alkylene-heterocycloalkyl group,
R′_{c} and R″ _{c}, independently of one another, represent a hydrogen atom or a linear or branched (C₁-C₆)alkyl,
R_{d} and Rₑ, independently of one another, represent a hydrogen atom, a linear or branched (C₁-C₆)alkyl group, a cycloalkyl group or a heterocycloalkyl group,
R_{f} represents a hydrogen atom, a halogen atom or a cyano group,
R′_{f} represents a hydrogen atom or a halogen atom,
R_{g} and Rₕ, independently of one another, represent a hydrogen atom, a linear or branched (C₁-C₆)alkyl group optionally substituted by one to three halogen atoms, a cycloalkyl group, a heterocycloalkyl group, or a -(C₁-C₆)alkylene-heterocycloalkyl,
Rᵢ, Rj, and Rₖ, independently of one another, represent a hydrogen atom, a linear or branched (C₁-C₆)alkyl group, or a -(C₁-C₆)alkylene-cycloalkyl group,
R₁ represents a hydrogen atom, a linear or branched (C₁-C₆)alkyl group or a linear or branched (C₁-C₆)alkylene-heterocycloalkyl group, Rm represents a hydrogen or a linear or branched (C₁-C₆)alkyl group.

115. The antibody-drug conjugate of claim 114, or a pharmaceutically acceptable salt thereof, wherein:
(i) the Bcl-2 inhibitor is represented by Formula (IV) or an enantiomer, a diastereoisomer, and/or a pharmaceutically acceptable salt of any one of the foregoing;
(ii) in Formula (IV), (a) A₁ represents a hydrogen atom or a methyl group; or (b) A₁ and A₂ both represent a methyl group;
(iii) in Formula (IV), T represents a methyl, aminomethyl, (morpholin-4-yl)methyl, (4-methylpiperazin-1-yl)methyl, 2-(morpholin-4-yl)ethyl, [2-(morpholin-4-yl)ethoxylmethyl, hydroxymethyl, [2-(dimethylamino)ethoxylmethyl, hexahydropyrazino[2,1-c1[1,41oxazin-8(1H)-ylmethyl, 1-oxa-6-azaspiro[3.31hept-6-ylmethyl, 3-(morpholin-4-yl)propyl or trifluoromethyl group; or
(iv) in Formula (IV), R₃ represents a group selected from phenyl, 1H-pyrazole, 1H-indole, 1H-indazole, pyridine, pyrimidine, 1H-pyrrolo[2,3-blpyridine, 2,3-dihydro-1H-pyrrolo[2,3-blpyridine, 1H-benzimidazole, 1H-pyrrole, 1H-pyrrolo[2,3-clpyridine, 1H-pyrrolo[3,2-blpyridine, 5H-pyrrolo[3,2-d1pyrimidine, thiophene, pyrazine, 1H-pyrazolo[3,4-blpyridine, 1,2-oxazole, and pyrazolo[1,5-alpyrimidine, those groups optionally having one or more substituents selected from halogen, linear or branched (C₁-C₆)alkyl, linear or branched (C₁-C₆)alkoxy, cyano, cyclopropyl, oxetane, tetrahydrofuran, —COO—CH₃, trideuteriomethyl, 2-(morpholin-4-yl)ethyl and 2-(morpholin-4-yl)ethoxy.

116-118. (canceled)

119. The antibody-drug conjugate of claim 114, wherein:
(i) the Bcl-2 inhibitor is represented by Formula (V) or an enantiomer, a diastereoisomer, and/or a pharmaceutically acceptable salt of any one of the foregoing;
(ii) the Bcl-2 inhibitor is represented by Formula (Va): 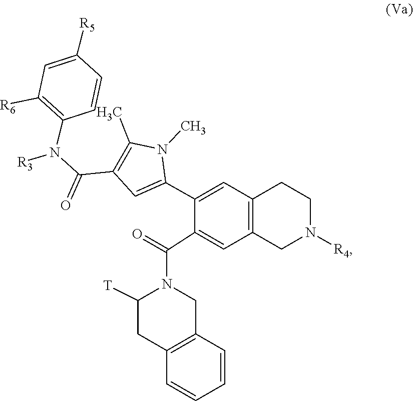
or an enantiomer, a diastereoisomer, and/or a pharmaceutically acceptable salt of any one of the foregoing;
optionally wherein:
(a) R₃ in Formula (V) or (Va) represents the following group: 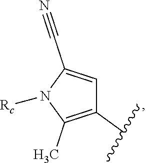
and R_{c} represents a group selected from: hydrogen, linear or branched (C₁-C₆)alkyl group optionally substituted by 1 to 3 halogen atoms, (C₁-C₆)alkylene-NR_{d}Rₑ, (C₁-C₆)alkylene-ORᵢ, cycloalkyl, heterocycloalkyl, and (C₁-C₆)alkylene-heterocycloalkyl group;
(b) R_{c} represents a methyl group; and/or
(c) R₄ in Formula (V) or (Va) represents the following group: 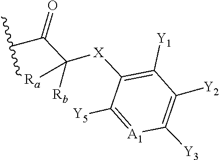
(iii) the Bcl-2 inhibitor is represented by Formula (Vb): 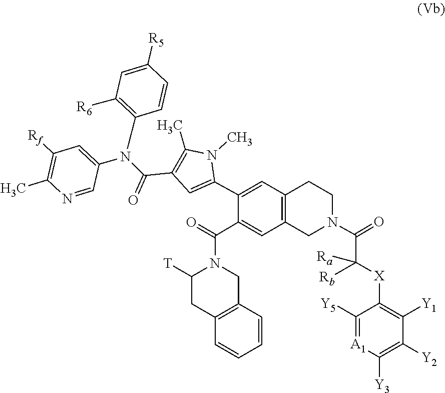
or an enantiomer, a diastereoisomer, and/or a pharmaceutically acceptable salt of any one of the foregoing; optionally wherein R_{c} in Formula (Vb) represents a methyl group; or
(iv) the Bcl-2 inhibitor is represented by Formula (Vc), (Vd), (Ve), (Vf), (Vg), (Vh), (Vi) or (Vj): 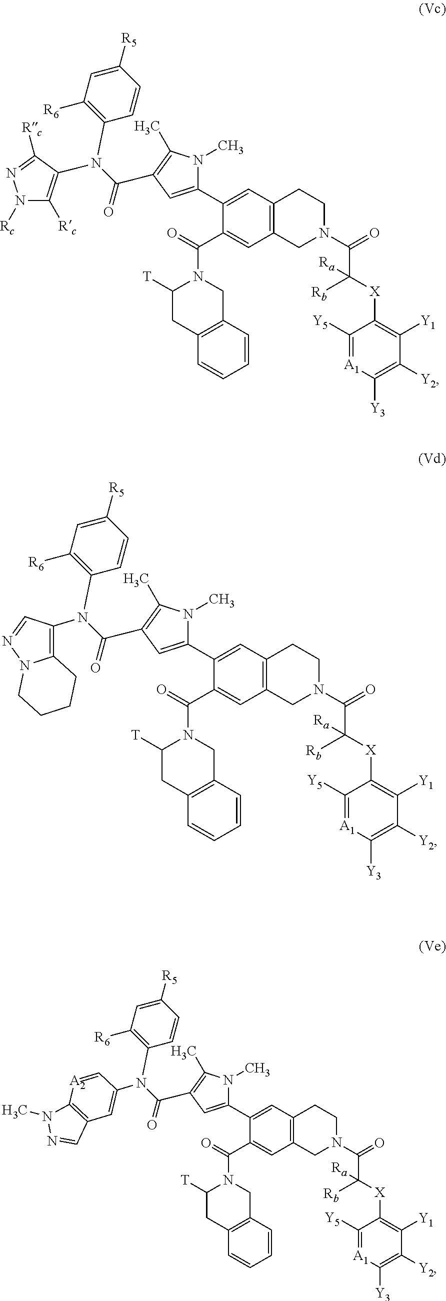 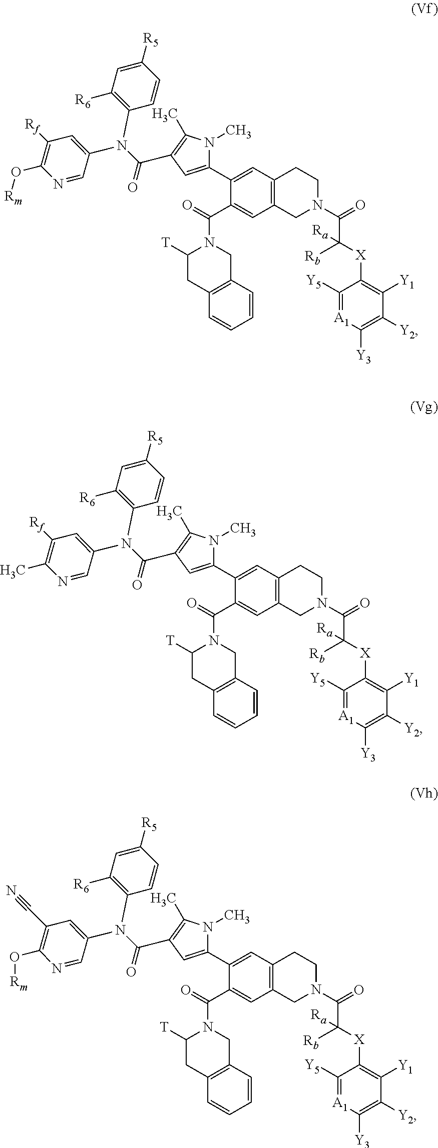 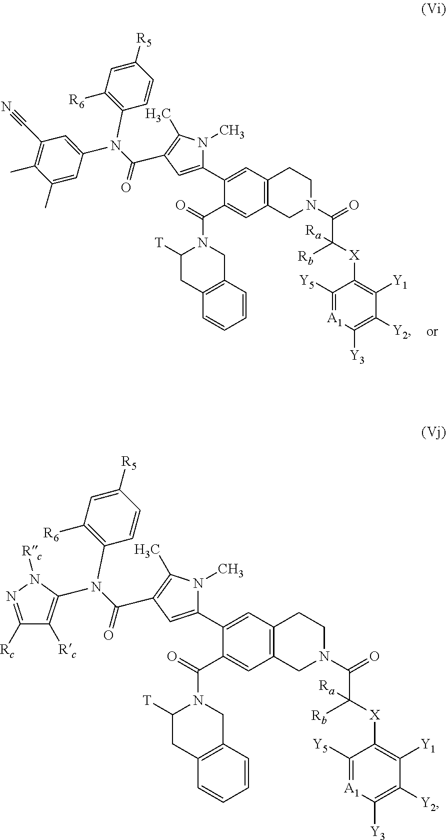
or an enantiomer, a diastereoisomer, and/or a pharmaceutically acceptable salt of any one of the foregoing.

120-126. (canceled)

127. The antibody-drug conjugate of claim 11, wherein in Formula (V), (Va), (Vb), (Vc), (Vd), (Ve), (Vf), (Vg), (Vh), (Vi) or (Vj):
(i) X represents a bond;
(ii) A₁ represents C—Y₄;
(iii) Rₐ and R_{b} both represent a hydrogen atom;
(iv) R₅ represents a hydrogen atom, a hydroxy group or a fluorine atom, preferably a hydroxy group; (v) R₆ represents a hydrogen atom, or a fluorine atom, preferably a hydrogen atom;
(vi) A₁ represents C—H and Y₂ represents a hydrogen atom;
(vii) Y₁ and Y₅ represent both a hydrogen atom, or: Y₁ and Y₅ represent a fluoro atom and a hydrogen atom, respectively;
(viii) Y₃ represents a —O-(C₁-C₆)alkylene-heterocycloalkyl group or a —O-(C₁-C₄)alkylene-Cy₃ group;
(ix) Y₃ represents a group selected from: 2-(morpholin-4-yl)ethoxy, 2-(oxan-4-yl)ethoxy, 2-(4-hydroxypiperidin-1-yl)ethoxy, 2-(4-cyclopropylpiperazin-1-yl)ethoxy, 2-[4-(2,2,2-trifluoroethyl)piperazin-1-yl]ethoxy, 2-[(9aS)-octahydropyrazino[2,1-c][1,4]oxazin-8-yl]ethoxy, 2-{2-[4-(2-{1,1-dioxo-1X⁶-thia-6-azaspiro[3.3]heptan-6-yl}ethoxy, 2-[2,6-dimethylmorpholin-4-yl]ethoxy, 2-[4-(2,2-difluoroethyl)piperazin-1-yl]ethoxy, 2-(3-fluoroazetidin-1-yl)ethoxy, 2-(3,3-difluoropyrrolidin-1-yl)ethoxy, 2-(4-fluoropiperidin-1-yl)ethoxy, 2-(thiomorpholin-4-yl)ethoxy, 2-(2-methylmorpholin-4-yl)ethoxy, 2-{6-oxa-9-azaspiro[4.5]decan-9-yl}ethoxy, 2-{4-oxa-7-azaspiro[2.5]octan-7-yl}ethoxy, 2-[4-(2-fluoroethyl)piperazin-1-yl]ethoxy, 2-(4-methylpiperazin-1-yl)ethoxy, 2-(2,2-dimethylmorpholin-4-yl)ethoxy, 2-(morpholin-4-yl)propoxy, [2-methyl-1-(morpholin-4-yl)propan-2-yl]oxy, 2-(3,3-dimethylmorpholin-4-yl)ethoxy, 2-(3-methylmorpholin-4-yl)ethoxy, 2-(1,4-dioxan-2-yl)ethoxy; preferably Y₃ represents a —O-(C₁-C₄)alkylene-Cy₃ group;
(x) the group: 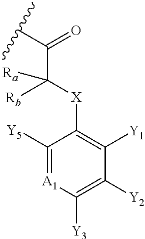
represents 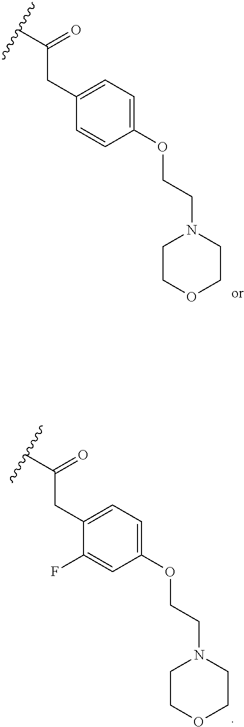 [SMILES: *C(=O)Cc1ccc(OCCN2CCOCC2)cc1 ; *C(=O)Cc1ccc(OCCN2CCOCC2)cc1F]
(xi) T represents a linear or branched (C₁-C₆)alkyl group or a (C₁-C₄)alkylene-NR₁R² group; and/or
(xii) T represents a group selected from: methyl group, (piperidin-1-yl)methyl, (morpholin-4-yl)methyl, (piperidin-1-yl)ethyl, [(3R)-3-fluoropyrrolidin-1-yl]methyl, (4-fluoropiperidin-1-yl)methyl, [methyl(propan-2-yl)amino]methyl, (azepan-l-yl)methyl, (pyrrolidin-1-yl)methyl, [(3S)-3-methylpiperidin-1-yl]methyl, [(3R)-3-methylpiperidin-1-yl]methyl, [(1RS,5SR)-3-azabicyclo[3.1.0]hexan-3-yl]methyl, [(2S)-2-methylpiperidin-1-yl]methyl, {6-azaspiro[2.5]octan-6-yl}methyl, (4,4-difluoropiperidin-1-yl)methyl, (diethylamino)methyl, (4-methylpiperidin-1-yl)methyl, [ethyl(propan-2-yl)amino]methyl, {5-azaspiro[2.3]hexan-5-yl}methyl, (3,3-dimethylpyrrolidin-1-yl)methyl, (diisopropylamino)methyl, [ethyl(isopropyl) amino]methyl, [(3R)-3-methylpyrrolidin-1-yl]methyl, [(3S)-3-methylpyrrolidin-1-yl]methyl, [(2S)-2-methylpyrrolidin-1-yl]methyl, 5-azaspiro[2.4]heptan-5-ylmethyl, 2-azaspiro[3.3]heptan-2-ylmethyl, and aminomethyl.

128-130. (canceled)

131. The antibody-drug conjugate of claim 114 wherein the Bcl-2 inhibitor is represented by any one of the following:

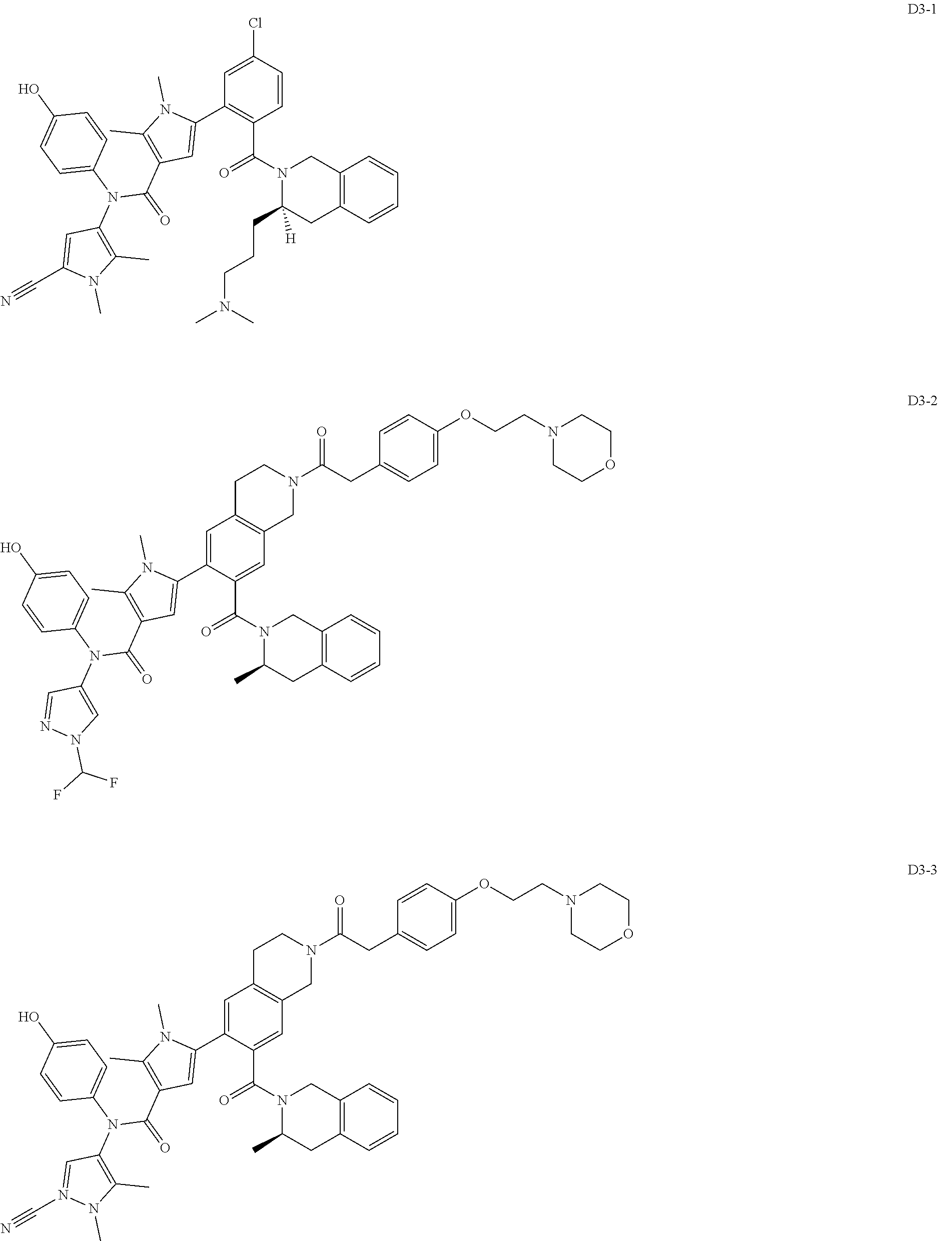

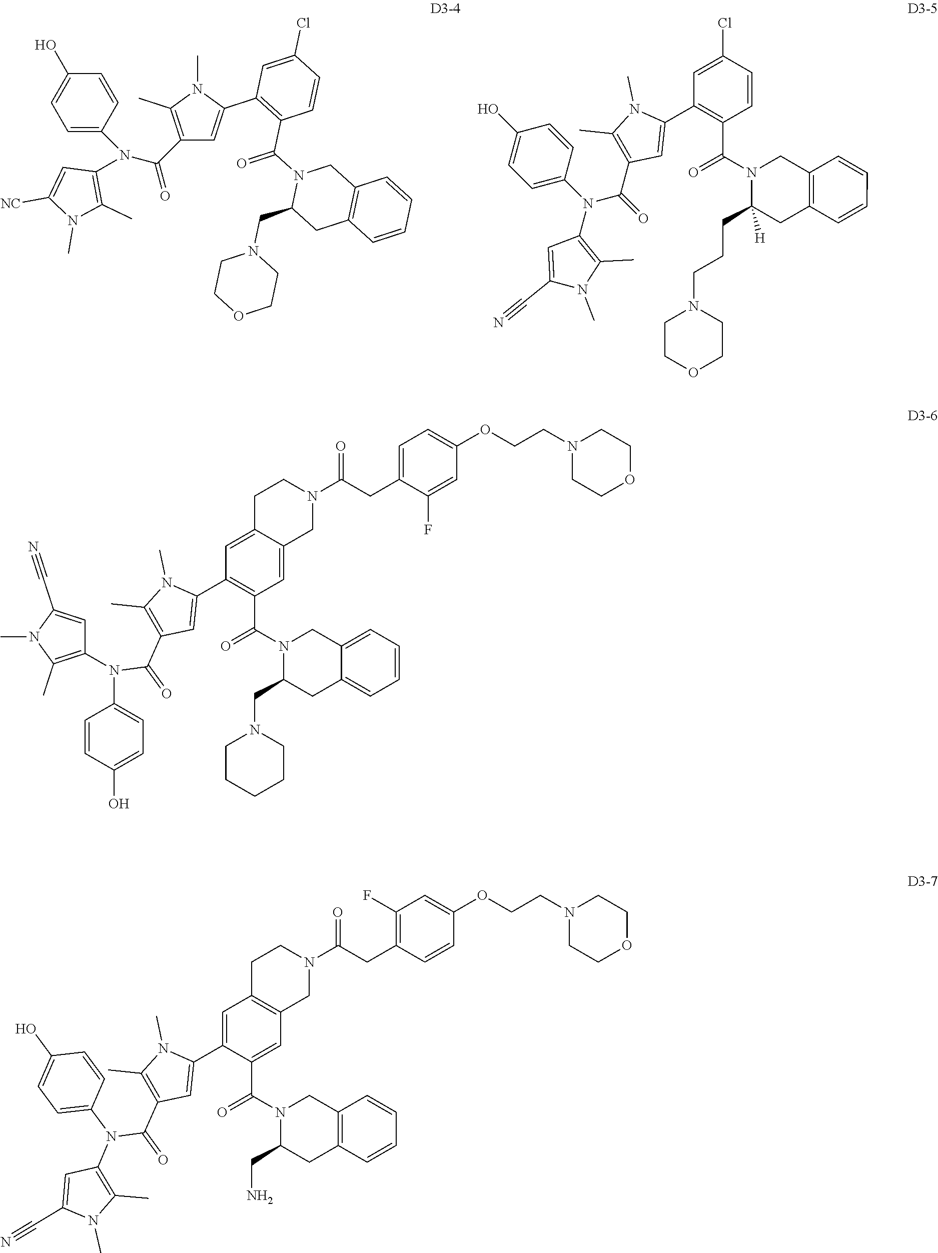 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C ; Cc1c(N(C(=O)c2cc(-c3cc(Cl)ccc3C(=O)N3Cc4ccccc4C[C@H]3CCCN3CCOCC3)n(C)c2C)c2ccc(O)cc2)cc(C#N)n1C ; Cc1c(N(C(=O)c2cc(-c3cc4c(cc3C(=O)N3Cc5ccccc5C[C@H]3CN3CCCCC3)CN(C(=O)Cc3ccc(OCCN5CCOCC5)cc3F)CC4)n(C)c2C)c2ccc(O)cc2)cc(C#N)n1C ; Cc1c(N(C(=O)c2cc(-c3cc4c(cc3C(=O)N3Cc5ccccc5C[C@H]3CN)CN(C(=O)Cc3ccc(OCCN5CCOCC5)cc3F)CC4)n(C)c2C)c2ccc(O)cc2)cc(C#N)n1C]

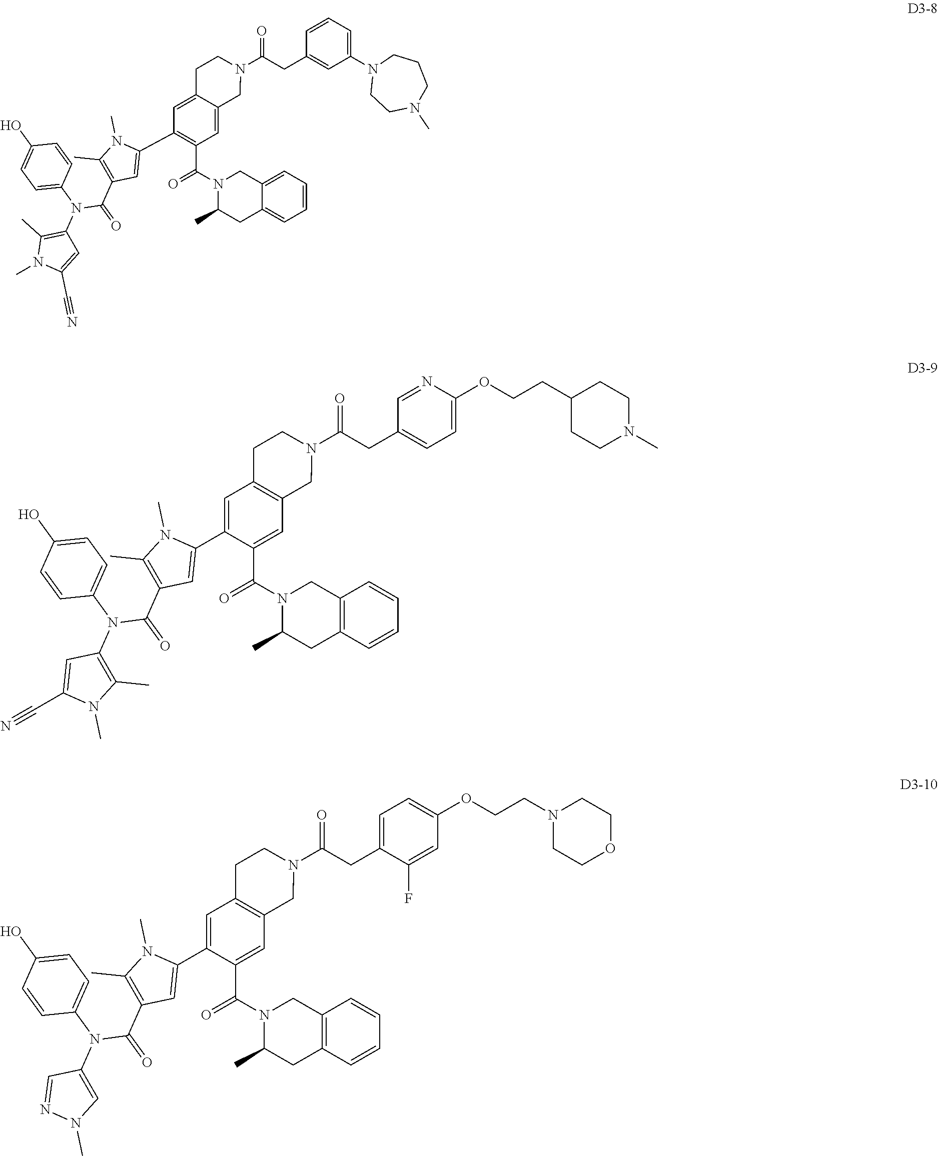

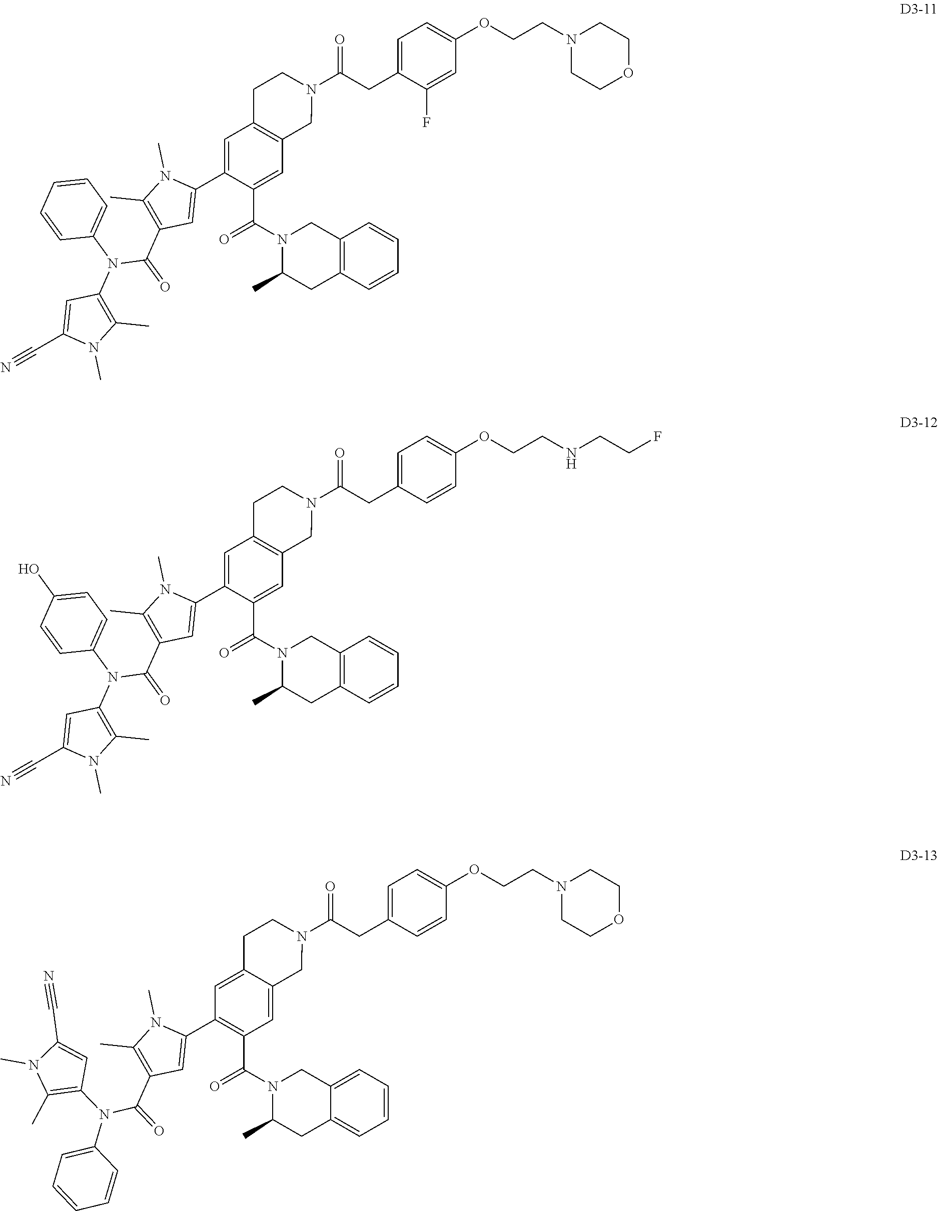 [SMILES: Cc1c(N(C(=O)c2cc(-c3cc4c(cc3C(=O)N3Cc5ccccc5C[C@H]3C)CN(C(=O)Cc3ccc(OCCN5CCOCC5)cc3F)CC4)n(C)c2C)c2ccccc2)cc(C#N)n1C ; Cc1c(N(C(=O)c2cc(-c3cc4c(cc3C(=O)N3Cc5ccccc5C[C@H]3C)CN(C(=O)Cc3ccc(OCCNCCF)cc3)CC4)n(C)c2C)c2ccc(O)cc2)cc(C#N)n1C ; Cc1c(N(C(=O)c2cc(-c3cc4c(cc3C(=O)N3Cc5ccccc5C[C@H]3C)CN(C(=O)Cc3ccc(OCCN5CCOCC5)cc3)CC4)n(C)c2C)c2ccccc2)cc(C#N)n1C]

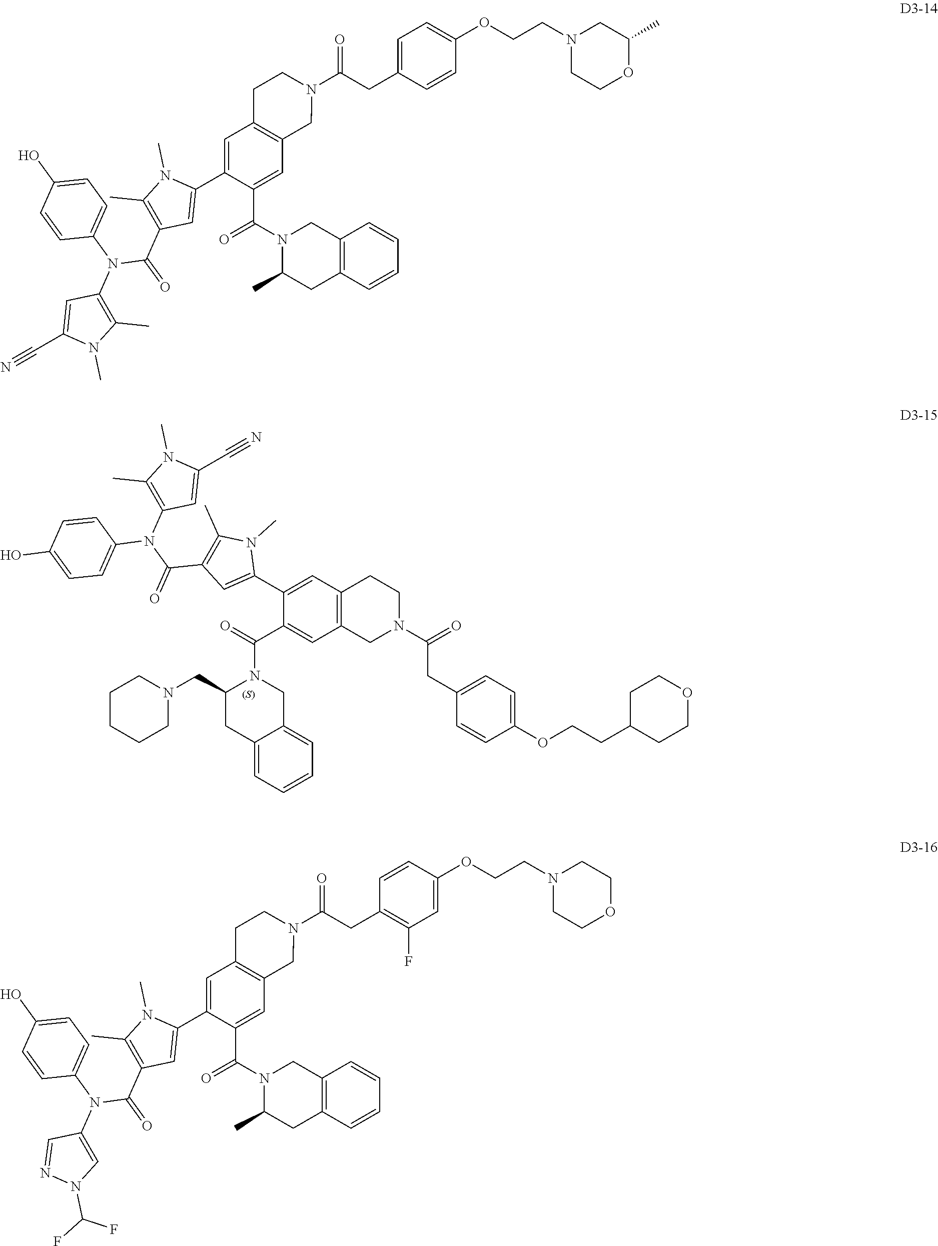

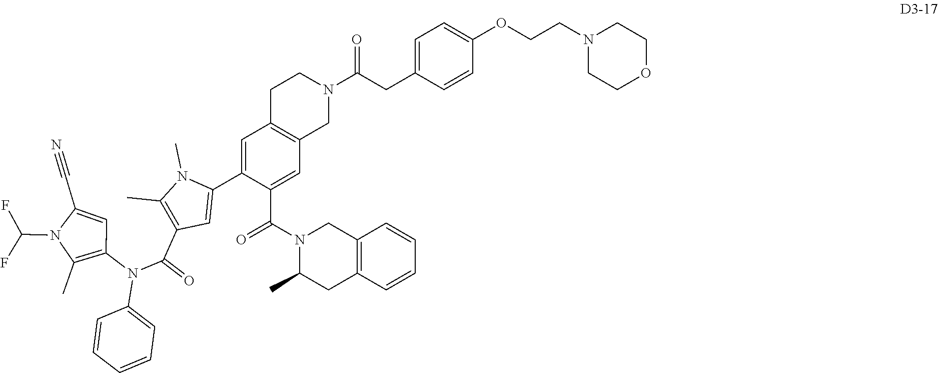 [SMILES: Cc1c(C(=O)N(c2ccccc2)c2cc(C#N)n(C(F)F)c2C)cc(-c2cc3c(cc2C(=O)N2Cc4ccccc4C[C@H]2C)CN(C(=O)Cc2ccc(OCCN4CCOCC4)cc2)CC3)n1C]

132. The antibody-drug conjugate of claim 78, wherein: (i) the topoisomerase 1 inhibitor is: 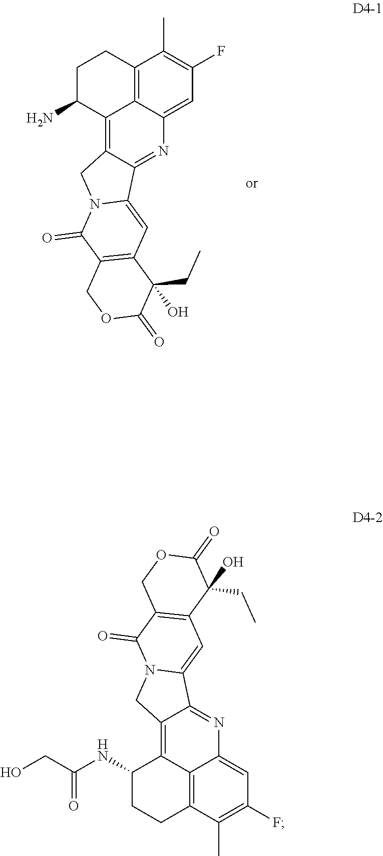 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3]
or (ii) the anti-mitotic drug is monomethyl auristatin E (MMAE) or a taxane; preferably wherein the taxane is selected from docetaxel, paclitaxel, or cabazitaxel.

133-134. (canceled)

135. The antibody-drug conjugate of claim 1, wherein;
(i) the antibody or antigen-binding fragment thereof binds to a target antigen on a cancer cell; optionally wherein:
(a) the target antigen is selected from BCMA, CD33, HER2, CD38, CD48, CD79b, PCAD, CD74, CD138, SLAMF7, CD123, CLL1, FLT3, CD7, CKIT, CD56, SEZ6, DLL3, DLK1, B7-H3, EGFR, CD71, EphA2, EPCAM, FOLR1, ENPP3, MET, AXL, SLC34A2, Nectin4, TROP2, LIV1, CD46, MSLN, F3, MUC16, SLC39A6, TFRC, TACSTD2, and GPNMB;
(b) the target antigen is selected from EGFR, CD7, HER2, EPCAM, FOLR1, ENPP3, MET, AXL, SLC34A2, Nectin4, MSLN, F3, MUC16, SLC39A6, TFRC, TACSTD2, and GPNMB; or
(c) the target antigen is selected from CD48, CD74, EphA2, PCAD, TROP2, B7-H3, or 5T4 or HER2; or
(ii) the antibody or antigen-binding fragment thereof is selected from Table D1; or
(iii) the antibody or antigen-binding fragment thereof comprises i) three heavy chain CDR sequences and three light chain CDR sequences selected from an antibody in Tables D3 and D8, ii) a heavy chain variable region sequence and a light chain variable region sequence selected from an antibody in Tables D2 and D8, or iii) a heavy chain sequence and light chain sequence selected from an antibody in Tables D4, D5, and D7.

136-138. (canceled)

139. The antibody-drug conjugate of claim 135, wherein:
(i) the antibody or antigen-binding fragment thereof is
(A) an anti-CD74 antibody comprising three heavy chain CDRs and three light chain CDRs selected from the group consisting of:
1) heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:268, light chain CDR2 (LCDR2) consisting of SEQ ID NO:264, and light chain CDR3 (LCDR3) consisting of SEQ ID NO:265;
2) heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174;
3) heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:269, light chain CDR2 (LCDR2) consisting of SEQ ID NO:264, and light chain CDR3 (LCDR3) consisting of SEQ ID NO:174;
4) heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174;
5) heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:263, light chain CDR2 (LCDR2) consisting of SEQ ID NO:264, and light chain CDR3 (LCDR3) consisting of SEQ ID NO:265;
6) heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174;
7) heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:215, light chain CDR2 (LCDR2) consisting of SEQ ID NO:264, and light chain CDR3 (LCDR3) consisting of SEQ ID NO:174; and
8) heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174;
(B) an anti-CD74 antibody comprising (1) a heavy chain variable region comprising the amino acid sequence of SEQ ID NO:153, and a light chain variable region comprising the amino acid sequence of SEQ ID NO:262, or (2) a heavy chain variable region comprising the amino acid sequence of SEQ ID NO:153, and a light chain variable region comprising the amino acid sequence of SEQ ID NO:267; or
(C) an anti-CD74 antibody comprising:
(1) the heavy chain amino acid sequence of SEQ ID NO:118 or a sequence that is at least 95% identical to SEQ ID NO:118, and the light chain amino acid sequence of SEQ ID NO:237 or a sequence that is at least 95% identical to SEQ ID NO:237;
(2) the heavy chain amino acid sequence of SEQ ID NO:236 or a sequence that is at least 95% identical to SEQ ID NO:236, and the light chain amino acid sequence of SEQ ID NO:237 or a sequence that is at least 95% identical to SEQ ID NO:237; or
(3) the heavy chain amino acid sequence of SEQ ID NO:118 or a sequence that is at least 95% identical to SEQ ID NO:118, and the light chain amino acid sequence of SEQ ID NO:239 or a sequence that is at least 95% identical to SEQ ID NO:239;
(ii) the antibody or antigen-binding fragment thereof is:
(A) an anti-CD48 antibody comprising three heavy chain CDRs and three light chain CDRs selected from the group consisting of:
1) heavy chain CDR1 (HCDR1) consisting of SEQ ID NO:271, heavy chain CDR2 (HCDR2) consisting of SEQ ID NO:272, heavy chain CDR3 (HCDR3) consisting of SEQ ID NO:273; light chain CDR1 (LCDR1) consisting of SEQ ID NO:281, light chain CDR2 (LCDR2) consisting of SEQ ID NO:282, and light chain CDR3 (LCDR3) consisting of SEQ ID NO:283;
2) heavy chain CDR1 (HCDR1) consisting of SEQ ID NO:274, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:285, and light chain CDR3 (LCDR3) consisting of SEQ ID NO:286;
3) heavy chain CDR1 (HCDR1) consisting of SEQ ID NO:276, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:287, light chain CDR2 (LCDR2) consisting of SEQ ID NO:282, and light chain CDR3 (LCDR3) consisting of SEQ ID NO:286;
4) heavy chain CDR1 (HCDR1) consisting of SEQ ID NO:279, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:288, and light chain CDR3 (LCDR3) consisting of SEQ ID NO:286; and
5) heavy chain CDR1 (HCDR1) consisting of SEQ ID NO:51, heavy chain CDR2 (HCDR2) consisting of SEQ ID NO:52, heavy chain CDR3 (HCDR3) consisting of SEQ ID NO:53; light chain CDR1 (LCDR1) consisting of SEQ ID NO:54, light chain CDR2 (LCDR2) consisting of SEQ ID NO:55, and light chain CDR3 (LCDR3) consisting of SEQ ID NO:56.
(B) an anti-CD48 antibody comprising (1) a heavy chain variable region comprising the amino acid sequence of SEQ ID NO:270, and a light chain variable region comprising the amino acid sequence of SEQ ID NO:280; or (2) a heavy chain variable region comprising the amino acid sequence of SEQ ID NO:13, and a light chain variable region comprising the amino acid sequence of SEQ ID NO:14; or
(C) an anti-CD48 antibody comprising (1) the heavy chain amino acid sequence of SEQ ID NO:240 or a sequence that is at least 95% identical to SEQ ID NO:240, and the light chain amino acid sequence of SEQ ID NO:243 or a sequence that is at least 95% identical to SEQ ID NO:243; (2) the heavy chain amino acid sequence of SEQ ID NO:242 or a sequence that is at least 95% identical to SEQ ID NO:242, and the light chain amino acid sequence of SEQ ID NO:243 or a sequence that is at least 95% identical to SEQ ID NO:243; or (3) the heavy chain amino acid sequence of SEQ ID NO:240 or a sequence that is at least 95% identical to SEQ ID NO:240, and the light chain amino acid sequence of SEQ ID NO:69 or a sequence that is at least 95% identical to SEQ ID NO:70;
(iii) the antibody or antigen-binding fragment thereof is:
(A) an anti-Her2 antibody comprising three heavy chain CDRs and three light chain CDRs selected from the group consisting of:
1) heavy chain CDR1 (HCDR1) consisting of SEQ ID NO:289, heavy chain CDR2 (HCDR2) consisting of SEQ ID NO:290, heavy chain CDR3 (HCDR3) consisting of SEQ ID NO:291; light chain CDR1 (LCDR1) consisting of SEQ ID NO:297, light chain CDR2 (LCDR2) consisting of SEQ ID NO:298, and light chain CDR3 (LCDR3) consisting of SEQ ID NO:299:
2) heavy chain CDR1 (HCDR1) consisting of SEQ ID NO:292, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44;
3) heavy chain CDR1 (HCDR1) consisting of SEQ ID NO:293, heavy chain CDR2 (HCDR2) consisting of SEQ ID NO:294, heavy chain CDR3 (HCDR3) consisting of SEQ ID NO:295; light chain CDR1 (LCDR1) consisting of SEQ ID NO:302, light chain CDR2 (LCDR2) consisting of SEQ ID NO:298, and light chain CDR3 (LCDR3) consisting of SEQ ID NO:44; and
4) heavy chain CDR1 (HCDR1) consisting of SEQ ID NO:39, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44;
(B) an anti-Her2 antibody comprising a heavy chain variable region comprising the amino acid sequence of SEQ ID NO:9, and a light chain variable region comprising the amino acid sequence of SEQ ID NO:296; or
(C) an anti-Her2 antibody comprising the heavy chain amino acid sequence of SEQ ID NO:245 or a sequence that is at least 95% identical to SEQ ID NO:245, and the light chain amino acid sequence of SEQ ID NO:66 or a sequence that is at least 95% identical to SEQ ID NO:66;
(iv) the antibody or antigen-binding fragment thereof is:
(A) an anti-PCAD antibody comprising three heavy chain CDRs and three light chain CDRs selected from the group consisting of:
1) heavy chain CDR1 (HCDR1) consisting of SEQ ID NO:304, heavy chain CDR2 (HCDR2) consisting of SEQ ID NO:305, heavy chain CDR3 (HCDR3) consisting of SEQ ID NO:306; light chain CDR1 (LCDR1) consisting of SEQ ID NO:312, light chain CDR2 (LCDR2) consisting of SEQ ID NO:313, and light chain CDR3 (LCDR3) consisting of SEQ ID NO:314;
2) heavy chain CDR1 (HCDR1) consisting of SEQ ID NO:307, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316;
3) heavy chain CDR1 (HCDR1) consisting of SEQ ID NO:309, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:317, light chain CDR2 (LCDR2) consisting of SEQ ID NO:313, and light chain CDR3 (LCDR3) consisting of SEQ ID NO:316; and
4) heavy chain CDR1 (HCDR1) consisting of SEQ ID NO:310, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316;
(B) an anti-PCAD antibody comprising a heavy chain variable region comprising the amino acid sequence of SEQ ID NO:303, and a light chain variable region comprising the amino acid sequence of SEQ ID NO:311; or
(C) an anti-PCAD antibody comprising the heavy chain amino acid sequence of SEQ ID NO:248 or a sequence that is at least 95% identical to SEQ ID NO:248, and the light chain amino acid sequence of SEQ ID NO:250 or a sequence that is at least 95% identical to SEQ ID NO:250;
(v) the antibody or antigen-binding fragment thereof is
(A) an anti-EphA2 antibody comprising three heavy chain CDRs and three light chain CDRs selected from the group consisting of:
1) heavy chain CDR1 (HCDR1) consisting of SEQ ID NO:319, heavy chain CDR2 (HCDR2) consisting of SEQ ID NO:320, heavy chain CDR3 (HCDR3) consisting of SEQ ID NO:321; light chain CDR1 (LCDR1) consisting of SEQ ID NO:330, light chain CDR2 (LCDR2) consisting of SEQ ID NO:331, and light chain CDR3 (LCDR3) consisting of SEQ ID NO:332;
2) heavy chain CDR1 (HCDR1) consisting of SEQ ID NO:322, heavy chain CDR2 (HCDR2) consisting of SEQ ID NO:323, heavy chain CDR3 (HCDR3) consisting of SEQ ID NO:324; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335;
3) heavy chain CDR1 (HCDR1) consisting of SEQ ID NO:325, heavy chain CDR2 (HCDR2) consisting of SEQ ID NO:326, heavy chain CDR3 (HCDR3) consisting of SEQ ID NO:327; light chain CDR1 (LCDR1) consisting of SEQ ID NO:336, light chain CDR2 (LCDR2) consisting of SEQ ID NO:331, and light chain CDR3 (LCDR3) consisting of SEQ ID NO:335; and
4) heavy chain CDR1 (HCDR1) consisting of SEQ ID NO:328, heavy chain CDR2 (HCDR2) consisting of SEQ ID NO:323, heavy chain CDR3 (HCDR3) consisting of SEQ ID NO:321; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335.
(B) an anti-EphA2 antibody comprising a heavy chain variable region comprising the amino acid sequence of SEQ ID NO:318, and a light chain variable region comprising the amino acid sequence of SEQ ID NO:329; or
(C) an anti-EphA2 antibody comprising the heavy chain amino acid sequence of SEQ ID NO:252 or a sequence that is at least 95% identical to SEQ ID NO:252, and the light chain amino acid sequence of SEQ ID NO:254 or a sequence that is at least 95% identical to SEQ ID NO:254; or
(vi) the antibody or antigen-binding fragment thereof is
(A) an anti-MET antibody comprising three heavy chain CDRs and three light chain CDRs selected from the group consisting of:
1) heavy chain CDR1 (HCDR1) consisting of SEQ ID NO:349, heavy chain CDR2 (HCDR2) consisting of SEQ ID NO:350, heavy chain CDR3 (HCDR3) consisting of SEQ ID NO:351; light chain CDR1 (LCDR1) consisting of SEQ ID NO:352, light chain CDR2 (LCDR2) consisting of SEQ ID NO:353, and light chain CDR3 (LCDR3) consisting of SEQ ID NO: 354;
2) heavy chain CDR1 (HCDR1) consisting of SEQ ID NO:355, heavy chain CDR2 (HCDR2) consisting of SEQ ID NO:356, heavy chain CDR3 (HCDR3) consisting of SEQ ID NO:357; light chain CDR1 (LCDR1) consisting of SEQ ID NO:358, light chain CDR2 (LCDR2) consisting of SEQ ID NO:359, and light chain CDR3 (LCDR3) consisting of SEQ ID NO: 360; and
3) heavy chain CDR1 (HCDR1) consisting of SEQ ID NO:361, heavy chain CDR2 (HCDR2) consisting of SEQ ID NO:362, heavy chain CDR3 (HCDR3) consisting of SEQ ID NO:363; light chain CDR1 (LCDR1) consisting of SEQ ID NO:364, light chain CDR2 (LCDR2) consisting of SEQ ID NO:365, and light chain CDR3 (LCDR3) consisting of SEQ ID NO: 366;
(B) an anti- MET antibody comprising a heavy chain variable region and a light chain variable region selected from the group consisting of:
1) a heavy chain variable region comprising the amino acid sequence of SEQ ID NO:339, and a light chain variable region comprising the amino acid sequence of SEQ ID NO:340;
2) a heavy chain variable region comprising the amino acid sequence of SEQ ID NO:341, and a light chain variable region comprising the amino acid sequence of SEQ ID NO:342; and
3) a heavy chain variable region comprising the amino acid sequence of SEQ ID NO:343, and a light chain variable region comprising the amino acid sequence of SEQ ID NO:344; or
(C) an anti-MET antibody comprising a heavy chain and a light chain selected from the group consisting of:
1) the heavy chain amino acid sequence of SEQ ID NO:367 or a sequence that is at least 95% identical to SEQ ID NO:367, and the light chain amino acid sequence of SEQ ID NO:368 or a sequence that is at least 95% identical to SEQ ID NO:368:
2) the heavy chain amino acid sequence of SEQ ID NO:369 or a sequence that is at least 95% identical to SEQ ID NO:369, and the light chain amino acid sequence of SEQ ID NO:370 or a sequence that is at least 95% identical to SEQ ID NO:370;
3) the heavy chain amino acid sequence of SEQ ID NO:371 or a sequence that is at least 95% identical to SEQ ID NO:371, and the light chain amino acid sequence of SEQ ID NO:372 or a sequence that is at least 95% identical to SEQ ID NO:372;
4) the heavy chain amino acid sequence of SEQ ID NO:373 or a sequence that is at least 95% identical to SEQ ID NO:373, and the light chain amino acid sequence of SEQ ID NO:374 or a sequence that is at least 95% identical to SEQ ID NO:374;
5) the heavy chain amino acid sequence of SEQ ID NO:375 or a sequence that is at least 95% identical to SEQ ID NO:375, and the light chain amino acid sequence of SEQ ID NO:370 or a sequence that is at least 95% identical to SEQ ID NO:370; and
the heavy chain amino acid sequence of SEQ ID NO:376 or a sequence that is at least 95% identical to SEQ ID NO:376, and the light chain amino acid sequence of SEQ ID NO:372 or a sequence that is at least 95% identical to SEQ ID NO:372.

140-156. (canceled)

157. The antibody-drug conjugate of claim 139, wherein the two antineoplastic payloads are Bcl-xL inhibitors.

158. The antibody-drug conjugate of claim 139, wherein:
(i) the antibody or antigen binding fragment thereof comprises one or more cysteine substitutions selected from E152C, S375C, or both E152C and S375C of the heavy chain of the antibody or antigen binding fragment thereof, wherein the position is numbered according to the EU system, or
(ii) the antibody or antigen binding fragment thereof comprises one or more Fc silencing mutations.

159. (canceled)

160. A composition comprising multiple copies of the antibody-drug conjugate of claim 1, wherein the average a of the antibody-drug conjugates in the composition is from about 1 to about 8, e.g., about 1 to about 6, about 1 to about 4, or about 1 to about 2.

161. A pharmaceutical composition comprising the antibody-drug conjugate of claim 1, and a pharmaceutically acceptable carrier.

162. A method of:
(i) treating a subject having or suspected of having a cancer,
(ii) reducing or inhibiting the growth of a tumor in a subject;
(iii) reducing or inhibiting a hematological cancer in a subject; or
(iv) reducing or slowing the expansion of a cancer cell population in a subject, comprising administering to the subject a therapeutically effective amount of the antibody-drug conjugate of claim 1, optionally wherein
(a) the cancer is a breast cancer, multiple mveloma, plasma cell mveloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer;
(b) the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, spleen cancer, pancreatic cancer, stomach cancer, colon cancer, or head and neck cancer;
(c) the hematological cancer is chronic lymphocytic leukemia (CLL), follicular lymphoma, mantle cell lymphoma, diffuse large B-cell lymphoma, acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMoL), Hodgkin's lymphoma, non-Hodgkin's lymphoma or myelodysplasia syndrome (MDS); or
(d) the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer.

163-182. (canceled)


SEQUENCE LISTING (438 sequences)

SEQ ID NO: 1 - protein, 121 aa, synthetic construct
  REGION 1..121: note = Synthetic
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 2 - protein, 107 aa, synthetic construct
  REGION 1..107: note = Synthetic
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 3 - protein, 118 aa, synthetic construct
  REGION 1..118: note = Synthetic
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 4 - protein, 112 aa, synthetic construct
  REGION 1..112: note = Synthetic
NIMLTQSPSSLAVSAGEKVTMSCKSSQSVFFSSSQKNYLAWYQQIPGQSPKLLIYWASTRESGVPDRFTGSGSGTDFTLIISSVQSEDLAIYYCHQYLSSRTFGGGTKLEIK

SEQ ID NO: 5 - protein, 116 aa, synthetic construct [sequence omitted]

SEQ ID NO: 6 - protein, 111 aa, synthetic construct [sequence omitted]

SEQ ID NO: 7 - protein, 123 aa, synthetic construct
  REGION 1..123: note = Synthetic
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSS

SEQ ID NO: 8 - protein, 106 aa, synthetic construct
  REGION 1..106: note = Synthetic
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIK

SEQ ID NO: 9 - protein, 120 aa, synthetic construct [sequence omitted]

SEQ ID NO: 10 - protein, 109 aa, synthetic construct
  REGION 1..109: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 11 - protein, 122 aa, synthetic construct [sequence omitted]

SEQ ID NO: 12 - protein, 106 aa, synthetic construct
  REGION 1..106: note = Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEI

SEQ ID NO: 13 - protein, 119 aa, synthetic construct
  REGION 1..119: note = Synthetic
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSS

SEQ ID NO: 14 - protein, 107 aa, synthetic construct
  REGION 1..107: note = Synthetic
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIK

SEQ ID NO: 15 - protein, 10 aa, synthetic construct
  REGION 1..10: note = Synthetic
GGTFSNYWMH

SEQ ID NO: 16 - protein, 17 aa, synthetic construct
  REGION 1..17: note = Synthetic
ATYRGHSDTYYNQKFKG

SEQ ID NO: 17 - protein, 12 aa, synthetic construct
  REGION 1..12: note = Synthetic
GAIYNGYDVLDN

SEQ ID NO: 18 - protein, 11 aa, synthetic construct
  REGION 1..11: note = Synthetic
SASQDISNYLN

SEQ ID NO: 19 - protein, 7 aa, synthetic construct
  REGION 1..7: note = Synthetic
YTSNLHS

SEQ ID NO: 20 - protein, 9 aa, synthetic construct
  REGION 1..9: note = Synthetic
QQYRKLPWT

SEQ ID NO: 21 - protein, 10 aa, synthetic construct
  REGION 1..10: note = Synthetic
GYTFTSYYIH

SEQ ID NO: 22 - protein, 17 aa, synthetic construct
  REGION 1..17: note = Synthetic
VIYPGNDDISYNQKFKG

SEQ ID NO: 23 - protein, 9 aa, synthetic construct
  REGION 1..9: note = Synthetic
EVRLRYFDV

SEQ ID NO: 24 - protein, 17 aa, synthetic construct
  REGION 1..17: note = Synthetic
KSSQSVFFSSSQKNYLA

SEQ ID NO: 25 - protein, 7 aa, synthetic construct
  REGION 1..7: note = Synthetic
WASTRES

SEQ ID NO: 26 - protein, 8 aa, synthetic construct
  REGION 1..8: note = Synthetic
HQYLSSRT

SEQ ID NO: 27 - protein, 10 aa, synthetic construct
  REGION 1..10: note = Synthetic
GYTITDSNIH

SEQ ID NO: 28 - protein, 17 aa, synthetic construct
  REGION 1..17: note = Synthetic
YIYPYNGGTDYNQKFKN

SEQ ID NO: 29 - protein, 7 aa, synthetic construct
  REGION 1..7: note = Synthetic
GNPWLAY

SEQ ID NO: 30 - protein, 15 aa, synthetic construct
  REGION 1..15: note = Synthetic
RASESLDNYGIRFLT

SEQ ID NO: 31 - protein, 7 aa, synthetic construct
  REGION 1..7: note = Synthetic
AASNQGS

SEQ ID NO: 32 - protein, 9 aa, synthetic construct
  REGION 1..9: note = Synthetic
QQTKEVPWS

SEQ ID NO: 33 - protein, 17 aa, synthetic construct
  REGION 1..17: note = Synthetic
TCAISGDSVSSQSAAWN

SEQ ID NO: 34 - protein, 18 aa, synthetic construct
  REGION 1..18: note = Synthetic
RIYYRSKWYNDYALSVKS

SEQ ID NO: 35 - protein, 11 aa, synthetic construct
  REGION 1..11: note = Synthetic
GEGYGREGFAI

SEQ ID NO: 36 - protein, 11 aa, synthetic construct
  REGION 1..11: note = Synthetic
RASQTISNTLA

SEQ ID NO: 37 - protein, 7 aa, synthetic construct
  REGION 1..7: note = Synthetic
AASNLQS

SEQ ID NO: 38 - protein, 8 aa, synthetic construct
  REGION 1..8: note = Synthetic
QQYLSWFT

SEQ ID NO: 39 - protein, 10 aa, synthetic construct
  REGION 1..10: note = Synthetic
GFNIKDTYIH

SEQ ID NO: 40 - protein, 17 aa, synthetic construct
  REGION 1..17: note = Synthetic
RIYPTNGYTRYADSVKG

SEQ ID NO: 41 - protein, 11 aa, synthetic construct
  REGION 1..11: note = Synthetic
WGGDGFYAMDV

SEQ ID NO: 42 - protein, 12 aa, synthetic construct
  REGION 1..12: note = Synthetic
RASQDVNTAVAW

SEQ ID NO: 43 - protein, 7 aa, synthetic construct
  REGION 1..7: note = Synthetic
SASFLES

SEQ ID NO: 44 - protein, 9 aa, synthetic construct
  REGION 1..9: note = Synthetic
QQHYTTPPT

SEQ ID NO: 45 - protein, 10 aa, synthetic construct
  REGION 1..10: note = Synthetic
GFTFNSFAMS

SEQ ID NO: 46 - protein, 17 aa, synthetic construct
  REGION 1..17: note = Synthetic
AISGSGGGTYYADSVKG

SEQ ID NO: 47 - protein, 13 aa, synthetic construct
  REGION 1..13: note = Synthetic
DKILWFGEPVFDY

SEQ ID NO: 48 - protein, 11 aa, synthetic construct
  REGION 1..11: note = Synthetic
RASQSVSSYLA

SEQ ID NO: 49 - protein, 7 aa, synthetic construct
  REGION 1..7: note = Synthetic
DASNRAT

SEQ ID NO: 50 - protein, 9 aa, synthetic construct
  REGION 1..9: note = Synthetic
QQRSNWPPT

SEQ ID NO: 51 - protein, 10 aa, synthetic construct
  REGION 1..10: note = Synthetic
GYTFTDFGMN

SEQ ID NO: 52 - protein, 17 aa, synthetic construct
  REGION 1..17: note = Synthetic
WINTFTGEPSYGNVFKG

SEQ ID NO: 53 - protein, 10 aa, synthetic construct
  REGION 1..10: note = Synthetic
RHGNGNVFDS

SEQ ID NO: 54 - protein, 11 aa, synthetic construct
  REGION 1..11: note = Synthetic
RASQSIGSNIH

SEQ ID NO: 55 - protein, 7 aa, synthetic construct
  REGION 1..7: note = Synthetic
YTSESIS

SEQ ID NO: 56 - protein, 9 aa, synthetic construct
  REGION 1..9: note = Synthetic
QQSNSWPLT

SEQ ID NO: 57 - protein, 451 aa, synthetic construct
  REGION 1..451: note = Synthetic
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
  REGION 1..214: note = Synthetic
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 448 aa, synthetic construct
  REGION 1..448: note = Synthetic
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 60 - protein, 219 aa, synthetic construct
  REGION 1..219: note = Synthetic
NIMLTQSPSSLAVSAGEKVTMSCKSSQSVFFSSSQKNYLAWYQQIPGQSPKLLIYWASTRESGVPDRFTGSGSGTDFTLIISSVQSEDLAIYYCHQYLSSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 446 aa, synthetic construct
  REGION 1..446: note = Synthetic
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 62 - protein, 218 aa, synthetic construct
  REGION 1..218: note = Synthetic
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 453 aa, synthetic construct
  REGION 1..453: note = Synthetic
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 64 - protein, 213 aa, synthetic construct
  REGION 1..213: note = Synthetic
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 451 aa, synthetic construct
  REGION 1..451: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 452 aa, synthetic construct
  REGION 1..452: note = Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 68 - protein, 214 aa, synthetic construct
  REGION 1..214: note = Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 449 aa, synthetic construct
  REGION 1..449: note = Synthetic
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 70 - protein, 214 aa, synthetic construct
  REGION 1..214: note = Synthetic
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 233 aa, synthetic construct
  REGION 1..233: note = Synthetic
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 72 - protein, 184 aa, synthetic construct
  REGION 1..184: note = Synthetic
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 73 - protein, 364 aa, synthetic construct
  REGION 1..364: note = Synthetic
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 74 - protein, 829 aa, synthetic construct
  REGION 1..829: note = Synthetic
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 75 - protein, 1255 aa, synthetic construct [sequence omitted]

SEQ ID NO: 76 - protein, 300 aa, synthetic construct
  REGION 1..300: note = Synthetic
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 77 - protein, 243 aa, synthetic construct
  REGION 1..243: note = Synthetic
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLT

SEQ ID NO: 78 - protein, 229 aa, synthetic construct
  REGION 1..229: note = Synthetic
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 79 - protein, 534 aa, synthetic construct
  REGION 1..534: note = Synthetic
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 80 - protein, 117 aa, synthetic construct [sequence omitted]

SEQ ID NO: 81 - protein, 111 aa, synthetic construct [sequence omitted]

SEQ ID NO: 82 - protein, 10 aa, synthetic construct
  REGION 1..10: note = Synthetic
GYTFSSYWIE

SEQ ID NO: 83 - protein, 17 aa, synthetic construct
  REGION 1..17: note = Synthetic
EILPGGGDTNYNEIFKG

SEQ ID NO: 84 - protein, 8 aa, synthetic construct
  REGION 1..8: note = Synthetic
RVPIRLDY

SEQ ID NO: 85 - protein, 18 aa, synthetic construct
  REGION 1..18: note = Synthetic
ITCKASQSVDYEGDSFLN

SEQ ID NO: 86 - protein, 7 aa, synthetic construct
  REGION 1..7: note = Synthetic
AASNLES

SEQ ID NO: 87 - protein, 9 aa, synthetic construct
  REGION 1..9: note = Synthetic
QQSNEDPLT

SEQ ID NO: 88 - protein, 447 aa, synthetic construct
  REGION 1..447: note = Synthetic
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 89 - protein, 218 aa, synthetic construct
  REGION 1..218: note = Synthetic
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 116 aa, synthetic construct
  REGION 1..116: note = Synthetic
QVQLVQSGGGVVQPGRSLRLACAASGLTVNNYAMHWVRQAPGKGLEWVAVISYDGNNKYYADSVKGRFTISRDNSKNTLYLQMNSLRAEDTAVYYCAKGGGYFDLWGRGTLVTVSS

SEQ ID NO: 91 - protein, 109 aa, synthetic construct
  REGION 1..109: note = Synthetic
QSVLTQPPSVSGAPGQRVTISCTGSSSNIGAGYDVHWYQQLPGTAPKLLIYGNNNRPSGVPDRFSGSKSGTSASLAITGLQAEDEADYYCSSYTSGTWLFGGGTKLTVL

SEQ ID NO: 92 - protein, 449 aa, synthetic construct
  REGION 1..449: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 93 - protein, 214 aa, synthetic construct
  REGION 1..214: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 448 aa, synthetic construct
  REGION 1..448: note = Synthetic
QVQLVQSGAEVKKPGASVKMSCKASGYTFTSYWMHWVRQAPGQGLEWIGAIYPGNSETGYAQKFQGRATLTADTSTSTAYMELSSLRSEDTAVYYCTRENWDPGFAFWGQGTLITVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 95 - protein, 218 aa, synthetic construct
  REGION 1..218: note = Synthetic
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 446 aa, synthetic construct
  REGION 1..446: note = Synthetic
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 97 - protein, 219 aa, synthetic construct
  REGION 1..219: note = Synthetic
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 448 aa, synthetic construct
  REGION 1..448: note = Synthetic
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 99 - protein, 218 aa, synthetic construct; identical to SEQ ID NO: 95
  REGION 1..218: note = Synthetic

SEQ ID NO: 100 - protein, 453 aa, synthetic construct
  REGION 1..453: note = Synthetic
QVQLQESGPGLVKPSQTLSLTCTVSGGSISSGGYYWSWIRQHPGKGLEWIGIIYYSGSTYYNPSLKSRVTISVDTSKNQFSLKLNSVTAADTAVFYCARVAIVTTIPGGMDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 101 - protein, 214 aa, synthetic construct
  REGION 1..214: note = Synthetic
EIVLTQSPDFQSVTPKEKVTITCRASQSIGISLHWYQQKPDQSPKLLIKYASQSFSGVPSRFSGSGSGTDFTLTINSLEAEDAATYYCHQSRSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 448 aa, synthetic construct
  REGION 1..448: note = Synthetic
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 103 - protein, 218 aa, synthetic construct
  REGION 1..218: note = Synthetic
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 446 aa, synthetic construct
  REGION 1..446: note = Synthetic
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 105 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 450 aa, synthetic construct
  REGION 1..450: note = Synthetic
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 107 - protein, 219 aa, synthetic construct
  REGION 1..219: note = Synthetic
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 447 aa, synthetic construct
  REGION 1..447: note = Synthetic
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 109 - protein, 214 aa, synthetic construct
  REGION 1..214: note = Synthetic
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 451 aa, synthetic construct
  REGION 1..451: note = Synthetic
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 111 - protein, 214 aa, synthetic construct
  REGION 1..214: note = Synthetic
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 450 aa, synthetic construct
  REGION 1..450: note = Synthetic
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 113 - protein, 219 aa, synthetic construct
  REGION 1..219: note = Synthetic
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 449 aa, synthetic construct
  REGION 1..449: note = Synthetic
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 115 - protein, 215 aa, synthetic construct
  REGION 1..215: note = Synthetic
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 450 aa, synthetic construct
  REGION 1..450: note = Synthetic
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 117 - protein, 217 aa, synthetic construct
  REGION 1..217: note = Synthetic
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 118 - protein, 450 aa, synthetic construct
  REGION 1..450: note = Synthetic
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 119 - protein, 219 aa, synthetic construct
  REGION 1..219: note = Synthetic
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 448 aa, synthetic construct
  REGION 1..448: note = Synthetic
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 121 - protein, 214 aa, synthetic construct
  REGION 1..214: note = Synthetic
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 446 aa, synthetic construct
  REGION 1..446: note = Synthetic
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 123 - protein, 214 aa, synthetic construct
  REGION 1..214: note = Synthetic
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 446 aa, synthetic construct
  REGION 1..446: note = Synthetic
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 125 - protein, 214 aa, synthetic construct
  REGION 1..214: note = Synthetic
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 1210 aa, Homo sapiens [sequence omitted]

SEQ ID NO: 127 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 128 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 129 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 130 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 131 - protein, 875 aa, Homo sapiens [sequence omitted]

SEQ ID NO: 132 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 133 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 134 - protein, 690 aa, Homo sapiens
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCDCPKCCRCSKCCEDLEEAQEGQDVPVKAPETFDNITISREAQGEVPASDSKTECTAL

SEQ ID NO: 135 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 136 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 137 - protein, 755 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSEHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 138 - protein, 572 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 139 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 140 - protein, 296 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 141 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 142 - protein, 14507 aa, Homo sapiens
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 143 - protein, 447 aa, synthetic construct
  REGION 1..447: note = Synthetic
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 144 - protein, 214 aa, synthetic construct
  REGION 1..214: note = Synthetic
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 4 aa, synthetic construct
  REGION 1..4: note = Synthetic
GFLG

SEQ ID NO: 146 - protein, 4 aa, synthetic construct
  REGION 1..4: note = Synthetic
GLFG

SEQ ID NO: 147 - protein, 4 aa, synthetic construct
  REGION 1..4: note = Synthetic
ALAL

SEQ ID NO: 148 - protein, 4 aa, synthetic construct
  REGION 1..4: note = Synthetic
GGGG

SEQ ID NO: 149 - protein, 4 aa, synthetic construct
  REGION 1..4: note = Synthetic
GFVG

SEQ ID NO: 150 - protein, 4 aa, synthetic construct
  REGION 1..4: note = Synthetic
GVFG

SEQ ID NO: 151 - protein, 116 aa, synthetic construct
  REGION 1..116: note = Synthetic
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 152 - protein, 107 aa, synthetic construct
  REGION 1..107: note = Synthetic
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 153 - protein, 120 aa, synthetic construct
  REGION 1..120: note = Synthetic
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 154 - protein, 112 aa, synthetic construct
  REGION 1..112: note = Synthetic
DIVMTQTPLSLPVTPGEPASISCRSSQSLVHRNQNTYLHWYLQKPGQSPQLLIYTVSNRFSGVPDRFSGSGSGTDFTLKISRVEAEDVGVYFCSQSSHVPPTFGQGTKLEIK

SEQ ID NO: 155 - protein, 121 aa, synthetic construct
  REGION 1..121: note = Synthetic
QVQLVQSGAEVKKPGASVKVSCKASGYTFTSYWINWVRQAPGQGLEWIGNIFPDTTTTNYNEKFKGRVTLTRDTSISTAYMELSRLRSDDTAVYYCAREYYDGTYDAMDYWGQGTLVTVSS

SEQ ID NO: 156 - protein, 107 aa, synthetic construct
  REGION 1..107: note = Synthetic
AIQMTQSPSSLSASVGDRVTITCKASQSVNNDVAWYQQKPGKAPKLLIYYASNRYTGVPSRFSGSGSGTDFTLTISSLQPEDFATYFCQQDYSSPRTFGQGTKLEIK

SEQ ID NO: 157 - protein, 118 aa, synthetic construct
  REGION 1..118: note = Synthetic
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 158 - protein, 112 aa, synthetic construct
  REGION 1..112: note = Synthetic
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 159 - protein, 118 aa, synthetic construct
  REGION 1..118: note = Synthetic
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 160 - protein, 107 aa, synthetic construct
  REGION 1..107: note = Synthetic
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 161 - protein, 117 aa, synthetic construct
  REGION 1..117: note = Synthetic
EVQLQQSGAELVKPGASVKLSCTASGFNIRDTYIHWVKQRPEQGLEWIGRIDPPNGNLKYDPKFQGKATITADTSSNTAYLQFSSLTSEDTAVYYCARSDGYSFAYWGQGTLVTVSS

SEQ ID NO: 162 - protein, 112 aa, synthetic construct
  REGION 1..112: note = Synthetic
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHVEYPFTFGSGTKLEIK

SEQ ID NO: 163 - protein, 117 aa, synthetic construct
  REGION 1..117: note = Synthetic
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSS

SEQ ID NO: 164 - protein, 112 aa, synthetic construct
  REGION 1..112: note = Synthetic
DIVMTQSPLSLPVTPGEPASISCRSSQSLVHSNRDTYLRWYLQKPGQSPQLLIYKVSNRFSGVPDRFSGSGSGTDFTLKISRVEAEDVGVYYCSQSTHVPYTFGGGTKVEIK

SEQ ID NO: 165 - protein, 122 aa, synthetic construct
  REGION 1..122: note = Synthetic
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 166 - protein, 106 aa, synthetic construct
  REGION 1..106: note = Synthetic
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 167 - protein, 128 aa, synthetic construct
  REGION 1..128: note = Synthetic
QVQLQQSGPGLVKPSQTLSLTCAISGDSVSSNSAAWSWIRQSPGRGLEWLGRIYYRSKWYNDYAVSVKSRITINPDTSKNQFSLQLNSVTPEDTAVYYCARLDHRYHEDTVYPGMDVWGQGTLVTVSS

SEQ ID NO: 168 - protein, 107 aa, synthetic construct
  REGION 1..107: note = Synthetic
DIELTQPPSVSVAPGQTARISCSGDNLPAYTVTWYQQKPGQAPVLVIYDDSDRPSGIPERFSGSNSGNTATLTISGTQAEDEADYYCASWDPSSGVVFGGGTKLTVL

SEQ ID NO: 169 - protein, 10 aa, synthetic construct
  REGION 1..10: note = Synthetic
GYTFTNYGVN

SEQ ID NO: 170 - protein, 17 aa, synthetic construct
  REGION 1..17: note = Synthetic
WINPNTGEPTFDDDFKG

SEQ ID NO: 171 - protein, 11 aa, synthetic construct
  REGION 1..11: note = Synthetic
SRGKNEAWFAY

SEQ ID NO: 172 - protein, 16 aa, synthetic construct
  REGION 1..16: note = Synthetic
RSSQSLVHRNQNTYLH

SEQ ID NO: 173 - protein, 7 aa, synthetic construct
  REGION 1..7: note = Synthetic
TVSNRFS

SEQ ID NO: 174 - protein, 9 aa, synthetic construct
  REGION 1..9: note = Synthetic
SQSSHVPPT

SEQ ID NO: 175 - protein, 10 aa, synthetic construct
  REGION 1..10: note = Synthetic
GYTFTSYWIN

SEQ ID NO: 176 - protein, 17 aa, synthetic construct
  REGION 1..17: note = Synthetic
NIFPDTTTTNYNEKFKG

SEQ ID NO: 177 - protein, 12 aa, synthetic construct
  REGION 1..12: note = Synthetic
EYYDGTYDAMDY

SEQ ID NO: 178 - protein, 11 aa, synthetic construct
  REGION 1..11: note = Synthetic
KASQSVNNDVA

SEQ ID NO: 179 - protein, 7 aa, synthetic construct
  REGION 1..7: note = Synthetic
YASNRYT

SEQ ID NO: 180 - protein, 9 aa, synthetic construct
  REGION 1..9: note = Synthetic
QQDYSSPRT

SEQ ID NO: 181 - protein, 10 aa, synthetic construct
  REGION 1..10: note = Synthetic
GFTFSSFGMH

SEQ ID NO: 182 - protein, 17 aa, synthetic construct
  REGION 1..17: note = Synthetic
YISSGSFTIYYADSVKG

SEQ ID NO: 183 - protein, 9 aa, synthetic construct
  REGION 1..9: note = Synthetic
MRKGYAMDY

SEQ ID NO: 184 - protein, 16 aa, synthetic construct
  REGION 1..16: note = Synthetic
RSSQIIIHSDGNTYLE

SEQ ID NO: 185 - protein, 7 aa, synthetic construct
  REGION 1..7: note = Synthetic
KVSNRFS

SEQ ID NO: 186 - protein, 9 aa, synthetic construct
  REGION 1..9: note = Synthetic
FQGSHVPHT

SEQ ID NO: 187 - protein, 10 aa, synthetic construct
  REGION 1..10: note = Synthetic
GYTFTNYGMN

SEQ ID NO: 188 - protein, 17 aa, synthetic construct
  REGION 1..17: note = Synthetic
WINTYTGEPTYADDFKG

SEQ ID NO: 189 - protein, 9 aa, synthetic construct
  REGION 1..9: note = Synthetic
IGDSSPSDY

SEQ ID NO: 190 - protein, 11 aa, synthetic construct
  REGION 1..11: note = Synthetic
KASQSVSNDVV

SEQ ID NO: 191 - protein, 9 aa, synthetic construct
  REGION 1..9: note = Synthetic
QQDYTSPWT

SEQ ID NO: 192 - protein, 10 aa, synthetic construct
  REGION 1..10: note = Synthetic
GFNIRDTYIH

SEQ ID NO: 193 - protein, 17 aa, synthetic construct
  REGION 1..17: note = Synthetic
RIDPPNGNLKYDPKFQG

SEQ ID NO: 194 - protein, 8 aa, synthetic construct
  REGION 1..8: note = Synthetic
SDGYSFAY

SEQ ID NO: 195 - protein, 16 aa, synthetic construct
  REGION 1..16: note = Synthetic
RSSKSLLHSNGNTYLY

SEQ ID NO: 196 - protein, 7 aa, synthetic construct
  REGION 1..7: note = Synthetic
RMSNLAS

SEQ ID NO: 197 - protein, 9 aa, synthetic construct
  REGION 1..9: note = Synthetic
MQHVEYPFT

SEQ ID NO: 198 - protein, 10 aa, synthetic construct
  REGION 1..10: note = Synthetic
GYTFSSYWMH

SEQ ID NO: 199 - protein, 17 aa, synthetic construct
  REGION 1..17: note = Synthetic
LIHPESGSTNYNEMFKN

SEQ ID NO: 200 - protein, 8 aa, synthetic construct
  REGION 1..8: note = Synthetic
GGRLYFDY

SEQ ID NO: 201 - protein, 16 aa, synthetic construct
  REGION 1..16: note = Synthetic
RSSQSLVHSNRDTYLR

SEQ ID NO: 202 - protein, 9 aa, synthetic construct
  REGION 1..9: note = Synthetic
SQSTHVPYT

SEQ ID NO: 203 - protein, 10 aa, synthetic construct
  REGION 1..10: note = Synthetic
GYTFTNYVMH

SEQ ID NO: 204 - protein, 17 aa, synthetic construct
  REGION 1..17: note = Synthetic
YINPYNDDVKYNEKFKG

SEQ ID NO: 205 - protein, 13 aa, synthetic construct
  REGION 1..13: note = Synthetic
WGYYGSPLYYFDY

SEQ ID NO: 206 - protein, 10 aa, synthetic construct
  REGION 1..10: note = Synthetic
RASSRLIYMH

SEQ ID NO: 207 - protein, 7 aa, synthetic construct
  REGION 1..7: note = Synthetic
ATSNLAS

SEQ ID NO: 208 - protein, 9 aa, synthetic construct
  REGION 1..9: note = Synthetic
QQWNSNPPT

SEQ ID NO: 209 - protein, 12 aa, synthetic construct
  REGION 1..12: note = Synthetic
GDSVSSNSAAWS

SEQ ID NO: 210 - protein, 18 aa, synthetic construct
  REGION 1..18: note = Synthetic
RIYYRSKWYNDYAVSVKS

SEQ ID NO: 211 - protein, 16 aa, synthetic construct
  REGION 1..16: note = Synthetic
LDHRYHEDTVYPGMDV

SEQ ID NO: 212 - protein, 11 aa, synthetic construct
  REGION 1..11: note = Synthetic
SGDNLPAYTVT

SEQ ID NO: 213 - protein, 7 aa, synthetic construct
  REGION 1..7: note = Synthetic
DDSDRPS

SEQ ID NO: 214 - protein, 10 aa, synthetic construct
  REGION 1..10: note = Synthetic
ASWDPSSGVV

SEQ ID NO: 215 - protein, 11 aa, synthetic construct
  REGION 1..11: note = Synthetic
QSLVHRNGNTY

SEQ ID NO: 216 - protein, 219 aa, synthetic construct
  REGION 1..219: note = Synthetic
DIVMTQTPLSLPVTPGEPASISCRSSQSLVHRNQNTYLHWYLQKPGQSPQLLIYTVSNRFSGVPDRFSGSGSGTDFTLKISRVEAEDVGVYFCSQSSHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 451 aa, synthetic construct
  REGION 1..451: note = Synthetic
QVQLVQSGAEVKKPGASVKVSCKASGYTFTSYWINWVRQAPGQGLEWIGNIFPDTTTTNYNEKFKGRVTLTRDTSISTAYMELSRLRSDDTAVYYCAREYYDGTYDAMDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 218 - protein, 214 aa, synthetic construct
  REGION 1..214: note = Synthetic
AIQMTQSPSSLSASVGDRVTITCKASQSVNNDVAWYQQKPGKAPKLLIYYASNRYTGVPSRFSGSGSGTDFTLTISSLQPEDFATYFCQQDYSSPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 448 aa, synthetic construct
  REGION 1..448: note = Synthetic
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 220 - protein, 219 aa, synthetic construct
  REGION 1..219: note = Synthetic
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 448 aa, synthetic construct
  REGION 1..448: note = Synthetic
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 222 - protein, 214 aa, synthetic construct
  REGION 1..214: note = Synthetic
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 447 aa, synthetic construct
  REGION 1..447: note = Synthetic
EVQLQQSGAELVKPGASVKLSCTASGFNIRDTYIHWVKQRPEQGLEWIGRIDPPNGNLKYDPKFQGKATITADTSSNTAYLQFSSLTSEDTAVYYCARSDGYSFAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 224 - protein, 219 aa, synthetic construct
  REGION 1..219: note = Synthetic
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHVEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 447 aa, synthetic construct
  REGION 1..447: note = Synthetic
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 226 - protein, 219 aa, synthetic construct
  REGION 1..219: note = Synthetic
DIVMTQSPLSLPVTPGEPASISCRSSQSLVHSNRDTYLRWYLQKPGQSPQLLIYKVSNRFSGVPDRFSGSGSGTDFTLKISRVEAEDVGVYYCSQSTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 452 aa, synthetic construct
  REGION 1..452: note = Synthetic
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 228 - protein, 213 aa, synthetic construct
  REGION 1..213: note = Synthetic
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 457 aa, synthetic construct
  REGION 1..457: note = Synthetic
QVQLQQSGPGLVKPSQTLSLTCAISGDSVSSNSAAWSWIRQSPGRGLEWLGRIYYRSKWYNDYAVSVKSRITINPDTSKNQFSLQLNSVTPEDTAVYYCARLDHRYHEDTVYPGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 230 - protein, 213 aa, synthetic construct
  REGION 1..213: note = Synthetic
DIELTQPPSVSVAPGQTARISCSGDNLPAYTVTWYQQKPGQAPVLVIYDDSDRPSGIPERFSGSNSGNTATLTISGTQAEDEADYYCASWDPSSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 231 - protein, 994 aa, synthetic construct
  REGION 1..994: note = Synthetic
MRPVALLLLPSLLALLAHGLSLEAPTVGKGQAPGIEETDGELTAAPTPEQPERGVHFVTTAPTLKLLNHHPLLEEFLQEGLEKGDEELRPALPFQPDPPAPFTPSPLPRLANQDSRPVFTSPTPAMAAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVEYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEKQLHPAGATIHFSCAPGYVLKGQASIKCVPGHPSHWSDPPPICRAASLDGFYNSRSLDVAKAPAASSTLDAAHIAAAIFLPLVAMVLLVGGVYFYFSRLQGKSSLQLPRPRPRPYNRITIESAFDNPTYETGSLSFAGDERI

SEQ ID NO: 232 - protein, 858 aa, synthetic construct
  REGION 1..858: note = Synthetic
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKASWTRPEKQETLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 233 - protein, 618 aa, synthetic construct
  REGION 1..618: note = Synthetic
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 234 - protein, 383 aa, synthetic construct
  REGION 1..383: note = Synthetic
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNRTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 235 - DNA, 1350 bp, synthetic construct
  misc_feature 1..1350: note = Synthetic
  source 1..1350: mol_type = other DNA
caggttcagctccagcagtctggcagcgagctgaaaaaacctggcgcctccgtgaaggtgtcctgcaaggcttctggctacacctttaccaactacggcgtgaactggatcaagcaggcccctggacaaggcctccaatggatgggctggatcaaccccaataccggcgagcccaccttcgacgacgatttcaagggcagattcgccttcagcctggacacctctgtgtccacagcctacctccagatcagcagcctgaaggccgatgataccgccgtgtacttctgctccagaagccggggaaagaacgaggcttggtttgcctattggggccagggcacactggtcaccgttagctctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 236 - protein, 450 aa, synthetic construct
  REGION 1..450: note = Synthetic
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 237 - protein, 219 aa, synthetic construct; identical to SEQ ID NO: 119
  REGION 1..219: note = Synthetic

SEQ ID NO: 238 - DNA, 657 bp, synthetic construct
  misc_feature 1..657: note = Synthetic
  source 1..657: mol_type = other DNA
gacattcagctgacacagagccctctgagcctgcctgttacactgggacagcctgccagcatcagctgtagaagcagccagagcctggtgcacagaaacggcaacacctacctgcactggttccagcagaggcctggccagtctcctagactgctgatctacaccgtgtccaacagattcagcggcgtgcccgatagattttccggcagcggctctggcaccgacttcaccctgaagattagcagagtggaagccgaggacgtgggcgtgtacttctgtagccagtctagccacgtgccacctacctttggcgccggaaccagactggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 239 - protein, 217 aa, synthetic construct
  REGION 1..217: note = Synthetic
DIVMTQTPLSLPVTPGEPASISCRSSQSLVHRNQNTYLHWYLQKPGQSPQLLIYTVSNRFSGVPDRFSGSGSGTDFTLKSRVEAEDVGVYFCSQSSHVPPTFGQGTKLEIKRTVAAPSVFIFPPSDEQLKSGTASVVCLLNNFYPREAKVQWKVDNALSGNSQESVTEQDSKDSTYSLSSTLTLSKADYEKHKVYACEVTHQGLSSPVTKSFNRGEC

SEQ ID NO: 240 - protein, 451 aa, synthetic construct
  REGION 1..451: note = Synthetic
EVQLLESGGGLVQPGGSLRLSCAASGFTFSSFAMSWVRQAPGKGLEWVSAISGFGGSTYYADSVKGRFTISRDNSKNTLYLQMNSLRAEDTAVYYCARQFWEDQPFYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 241 - DNA, 1353 bp, synthetic construct
  misc_feature 1..1353: note = Synthetic
  source 1..1353: mol_type = other DNA
gaggtgcagctgctggagagcggcggcggcctggtgcagcccggcggcagcctgcggctgagctgcgccgccagcggcttcaccttcagcagcttcgccatgagctgggtgcggcaggcccccggcaagggcctggagtgggtgagcgccatcagcggcttcggcggcagcacctactacgccgacagcgtgaagggccggttcaccatcagccgggacaacagcaagaacaccctgtacctgcagatgaacagcctgcgggccgaggacaccgccgtgtactactgcgcccggcagttctgggaggaccagcccttctacttcgactactggggccagggcaccctggtgaccgtgagcagcgccagcaccaagggccccagcgtgttccccctggcccccagcagcaagagcaccagcggcggcaccgccgccctgggctgcctggtgaaggactacttcccctgccccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctacccctgcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 242 - protein, 451 aa, synthetic construct
  REGION 1..451: note = Synthetic
EVQLLESGGGLVQPGGSLRLSCAASGFTFSSFAMSWVRQAPGKGLEWVSAISGFGGSTYYADSVKGRFTISRDNSKNTLYLQMNSLRAEDTAVYYCARQFWEDQPFYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 243 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - DNA, 642 bp, synthetic construct
  misc_feature 1..642: note = Synthetic
  source 1..642: mol_type = other DNA
gacatccagatgacccagagccccagcagcctgagcgccagcgtgggcgaccgggtgaccatcacctgccgggccagccagagcatcagcagctacctgaactggtaccagcagaagcccggcaaggcccccaagctgctgatctacgccgccagcagcctgcagagcggcgtgcccagccggttcagcggcagcggcagcggcaccgacttcaccctgaccatcagcagcctgcagcccgaggacttcgccacctactactgccagcagagctacagcacccccctgaccttcggccagggcaccaaggtggagatcaagcggaccgtggccgcccccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccggggcgagtgc

SEQ ID NO: 245 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 246 - DNA, 1350 bp, synthetic construct
  misc_feature 1..1350: note = Synthetic
  source 1..1350: mol_type = other DNA
gaggtgcagctggtggagagcggcggcggcctggtgcagcccggcggcagcctgcggctgagctgcgccgccagcggcttcaacatcaaggacacctacatccactgggtgcggcaggcccccggcaagggcctggagtgggtggcccggatctaccccaccaacggctacacccggtacgccgacagcgtgaagggccggttcaccatcagcgccgacaccagcaagaacaccgcctacctgcagatgaacagcctgcgggccgaggacaccgccgtgtactactgcagccggtggggcggcgacggcttctacgccatggactactggggccagggcaccctggtgaccgtgagcagcgccagcaccaagggccccagcgtgttccccctggcccccagcagcaagagcaccagcggcggcaccgccgccctgggctgcctggtgaaggactacttcccctgccccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctacccctgcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 247 - DNA, 642 bp, synthetic construct
  misc_feature 1..642: note = Synthetic
  source 1..642: mol_type = other DNA
gacatccagatgacccagagccccagcagcctgagcgccagcgtgggcgaccgggtgaccatcacctgccgggccagccaggacgtgaacaccgccgtggcctggtaccagcagaagcccggcaaggcccccaagctgctgatctacagcgccagcttcctgtacagcggcgtgcccagccggttcagcggcagccggagcggcaccgacttcaccctgaccatcagcagcctgcagcccgaggacttcgccacctactactgccagcagcactacaccaccccccccaccttcggccagggcaccaaggtggagatcaagcggaccgtggccgcccccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccggggcgagtgc

SEQ ID NO: 248 - protein, 448 aa, synthetic construct
  REGION 1..448: note = Synthetic
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - DNA, 1344 bp, synthetic construct
  misc_feature 1..1344: note = Synthetic
  source 1..1344: mol_type = other DNA
gaggtgcagctggtgcagagcggcgccgaggtgaagaagcccggcgagagcctgaagatcagctgcaaggtgagcggctacaccttcaccgaccacaccatccactggatgcggcagatgcccggcaagggcctggagtggatgggctacatctacccccggagcggcagcatcaactacaacgagaagttcaagggccaggtgaccatcagcgccgacaagagcagcagcaccgcctacctgcagtggagcagcctgaaggccagcgacaccgccatgtactactgcgcccggcggaacctgttcctgcccatggagtactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 250 - protein, 220 aa, synthetic construct
  REGION 1..220: note = Synthetic
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - DNA, 660 bp, synthetic construct
  misc_feature 1..660: note = Synthetic
  source 1..660: mol_type = other DNA
gacatcgtgatgacccagacccccctgagcctgcccgtgacccccggcgagcccgccagcatcagctgccggagcagccagagcctgctgagcagcggcgaccagaagaactacctgacctggtacctgcagaagcccggccagagcccccagctgctgatctactgggccagcacccgggagagcggcgtgcccgaccggttcagcggcagcggcagcggcaccgacttcaccctgaagatcagccgggtggaggccgaggacgtgggcgtgtactactgccagaacgactaccggtaccccctgaccttcggccagggcaccaagctggagatcaagcggaccgtggccgcccccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccggggcgagtgc

SEQ ID NO: 252 - protein, 458 aa, synthetic construct
  REGION 1..458: note = Synthetic
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 253 - DNA, 1374 bp, synthetic construct
  misc_feature 1..1374: note = Synthetic
  source 1..1374: mol_type = other DNA
gaagttcagctgcttgaatctggcggcggactggttcaacctggcggatctctgagactgagctgtgccgccagcggcttcaccttcagccactatatgatggcctgggtccgacaggcccctggcaaaggacttgaatgggtgtccagaatcggcccctctggcggccctacacactacgctgattctgtgaagggcagattcaccatcagccgggacaacagcaagaacaccctgtacctgcagatgaacagcctgagagccgaggacaccgccgtgtattactgtgccggctacgacagcggctacgattatgtggctgtggccggacctgccgagtactttcagcattggggacagggcaccctggtcaccgttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 254 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - DNA, 642 bp, synthetic construct
  misc_feature 1..642: note = Synthetic
  source 1..642: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtacaacagctacagccggacctttggccagggaacaaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 256 - protein, 7 aa, synthetic construct
  REGION 1..7: note = Synthetic
GYTFTNY

SEQ ID NO: 257 - protein, 6 aa, synthetic construct
  REGION 1..6: note = Synthetic
NPNTGE

SEQ ID NO: 258 - protein, 5 aa, synthetic construct
  REGION 1..5: note = Synthetic
NYGVN

SEQ ID NO: 259 - protein, 8 aa, synthetic construct
  REGION 1..8: note = Synthetic
GYTFTNYG

SEQ ID NO: 260 - protein, 8 aa, synthetic construct
  REGION 1..8: note = Synthetic
INPNTGEP

SEQ ID NO: 261 - protein, 13 aa, synthetic construct
  REGION 1..13: note = Synthetic
SRSRGKNEAWFAY

SEQ ID NO: 262 - protein, 112 aa, synthetic construct
  REGION 1..112: note = Synthetic
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 263 - protein, 12 aa, synthetic construct
  REGION 1..12: note = Synthetic
SQSLVHRNGNTY

SEQ ID NO: 264 - skipped

SEQ ID NO: 265 - protein, 6 aa, synthetic construct
  REGION 1..6: note = Synthetic
SSHVPP

SEQ ID NO: 266 - protein, 16 aa, synthetic construct
  REGION 1..16: note = Synthetic
RSSQSLVHRNGNTYLH

SEQ ID NO: 267 - protein, 111 aa, synthetic construct
  REGION 1..111: note = Synthetic
DIVMTQTPLSLPVTPGEPASISCRSSQSLVHRNQNTYLHWYLQKPGQSPQLLIYTVSNRFSGVPDRFSGSGSGTDFTLKSRVEAEDVGVYFCSQSSHVPPTFGQGTKLEIK

SEQ ID NO: 268 - protein, 12 aa, synthetic construct
  REGION 1..12: note = Synthetic
SQSLVHRNQNTY

SEQ ID NO: 269 - protein, 11 aa, synthetic construct
  REGION 1..11: note = Synthetic
QSLVHRNQNTY

SEQ ID NO: 270 - protein, 121 aa, synthetic construct
  REGION 1..121: note = Synthetic
EVQLLESGGGLVQPGGSLRLSCAASGFTFSSFAMSWVRQAPGKGLEWVSAISGFGGSTYYADSVKGRFTISRDNSKNTLYLQMNSLRAEDTAVYYCARQFWEDQPFYFDYWGQGTLVTVSS

SEQ ID NO: 271 - protein, 7 aa, synthetic construct
  REGION 1..7: note = Synthetic
GFTFSSF

SEQ ID NO: 272 - protein, 6 aa, synthetic construct
  REGION 1..6: note = Synthetic
SGFGGS

SEQ ID NO: 273 - protein, 12 aa, synthetic construct
  REGION 1..12: note = Synthetic
QFWEDQPFYFDY

SEQ ID NO: 274 - protein, 5 aa, synthetic construct
  REGION 1..5: note = Synthetic
SFAMS

SEQ ID NO: 275 - protein, 17 aa, synthetic construct
  REGION 1..17: note = Synthetic
AISGFGGSTYYADSVKG

SEQ ID NO: 276 - protein, 8 aa, synthetic construct
  REGION 1..8: note = Synthetic
GFTFSSFA

SEQ ID NO: 277 - protein, 8 aa, synthetic construct
  REGION 1..8: note = Synthetic
ISGFGGST

SEQ ID NO: 278 - protein, 14 aa, synthetic construct
  REGION 1..14: note = Synthetic
ARQFWEDQPFYFDY

SEQ ID NO: 279 - protein, 10 aa, synthetic construct
  REGION 1..10: note = Synthetic
GFTFSSFAMS

SEQ ID NO: 280 - protein, 107 aa, synthetic construct [sequence omitted]

SEQ ID NO: 281 - protein, 7 aa, synthetic construct
  REGION 1..7: note = Synthetic
SQSISSY

SEQ ID NO: 282 - skipped

SEQ ID NO: 283 - protein, 6 aa, synthetic construct
  REGION 1..6: note = Synthetic
SYSTPL

SEQ ID NO: 284 - protein, 11 aa, synthetic construct
  REGION 1..11: note = Synthetic
RASQSISSYLN

SEQ ID NO: 285 - protein, 7 aa, synthetic construct
  REGION 1..7: note = Synthetic
AASSLQS

SEQ ID NO: 286 - protein, 9 aa, synthetic construct
  REGION 1..9: note = Synthetic
QQSYSTPLT

SEQ ID NO: 287 - protein, 6 aa, synthetic construct
  REGION 1..6: note = Synthetic
QSISSY

SEQ ID NO: 288 - protein, 7 aa, synthetic construct
  REGION 1..7: note = Synthetic
AASSLQS

SEQ ID NO: 289 - protein, 7 aa, synthetic construct
  REGION 1..7: note = Synthetic
GFNIKDT

SEQ ID NO: 290 - protein, 6 aa, synthetic construct
  REGION 1..6: note = Synthetic
YPTNGY

SEQ ID NO: 291 - protein, 11 aa, synthetic construct
  REGION 1..11: note = Synthetic
WGGDGFYAMDY

SEQ ID NO: 292 - protein, 5 aa, synthetic construct
  REGION 1..5: note = Synthetic
DTYIH

SEQ ID NO: 293 - protein, 8 aa, synthetic construct
  REGION 1..8: note = Synthetic
GFNIKDTY

SEQ ID NO: 294 - protein, 8 aa, synthetic construct
  REGION 1..8: note = Synthetic
IYPTNGYT

SEQ ID NO: 295 - protein, 13 aa, synthetic construct
  REGION 1..13: note = Synthetic
SRWGGDGFYAMDY

SEQ ID NO: 296 - protein, 107 aa, synthetic construct [sequence omitted]

SEQ ID NO: 297 - protein, 7 aa, synthetic construct
  REGION 1..7: note = Synthetic
SQDVNTA

SEQ ID NO: 298 - skipped

SEQ ID NO: 299 - protein, 6 aa, synthetic construct
  REGION 1..6: note = Synthetic
HYTTPP

SEQ ID NO: 300 - protein, 11 aa, synthetic construct
  REGION 1..11: note = Synthetic
RASQDVNTAVA

SEQ ID NO: 301 - protein, 7 aa, synthetic construct
  REGION 1..7: note = Synthetic
SASFLYS

SEQ ID NO: 302 - protein, 6 aa, synthetic construct
  REGION 1..6: note = Synthetic
QDVNTA

SEQ ID NO: 303 - protein, 118 aa, synthetic construct
  REGION 1..118: note = Synthetic
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSS

SEQ ID NO: 304 - protein, 7 aa, synthetic construct
  REGION 1..7: note = Synthetic
GYTFTDH

SEQ ID NO: 305 - protein, 6 aa, synthetic construct
  REGION 1..6: note = Synthetic
YPRSGS

SEQ ID NO: 306 - protein, 9 aa, synthetic construct
  REGION 1..9: note = Synthetic
RNLFLPMEY

SEQ ID NO: 307 - protein, 5 aa, synthetic construct
  REGION 1..5: note = Synthetic
DHTIH

SEQ ID NO: 308 - protein, 17 aa, synthetic construct
  REGION 1..17: note = Synthetic
YIYPRSGSINYNEKFKG

SEQ ID NO: 309 - protein, 8 aa, synthetic construct
  REGION 1..8: note = Synthetic
GYTFTDHT

SEQ ID NO: 310 - protein, 10 aa, synthetic construct
  REGION 1..10: note = Synthetic
GYTFTDHTIH

SEQ ID NO: 311 - protein, 113 aa, synthetic construct
  REGION 1..113: note = Synthetic
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEIK

SEQ ID NO: 312 - protein, 13 aa, synthetic construct
  REGION 1..13: note = Synthetic
SQSLLSSGDQKNY

SEQ ID NO: 313 - skipped

SEQ ID NO: 314 - protein, 6 aa, synthetic construct
  REGION 1..6: note = Synthetic
DYRYPL

SEQ ID NO: 315 - protein, 17 aa, synthetic construct
  REGION 1..17: note = Synthetic
RSSQSLLSSGDQKNYLT

SEQ ID NO: 316 - protein, 9 aa, synthetic construct
  REGION 1..9: note = Synthetic
QNDYRYPLT

SEQ ID NO: 317 - protein, 12 aa, synthetic construct
  REGION 1..12: note = Synthetic
QSLLSSGDQKNY

SEQ ID NO: 318 - protein, 128 aa, synthetic construct
  REGION 1..128: note = Synthetic
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 319 - protein, 7 aa, synthetic construct
  REGION 1..7: note = Synthetic
GFTFSHY

SEQ ID NO: 320 - protein, 6 aa, synthetic construct
  REGION 1..6: note = Synthetic
GPSGGP

SEQ ID NO: 321 - protein, 19 aa, synthetic construct
  REGION 1..19: note = Synthetic
YDSGYDYVAVAGPAEYFQH

SEQ ID NO: 322 - protein, 5 aa, synthetic construct
  REGION 1..5: note = Synthetic
HYMMA

SEQ ID NO: 323 - protein, 17 aa, synthetic construct
  REGION 1..17: note = Synthetic
RIGPSGGPTHYADSVKG

SEQ ID NO: 324 - protein, 19 aa, synthetic construct
  REGION 1..19: note = Synthetic
YDSGYDYVAVAGPAEYFQH

SEQ ID NO: 325 - protein, 8 aa, synthetic construct
  REGION 1..8: note = Synthetic
GFTFSHYM

SEQ ID NO: 326 - protein, 8 aa, synthetic construct
  REGION 1..8: note = Synthetic
IGPSGGPT

SEQ ID NO: 327 - protein, 21 aa, synthetic construct
  REGION 1..21: note = Synthetic
AGYDSGYDYVAVAGPAEYFQH

SEQ ID NO: 328 - protein, 10 aa, synthetic construct
  REGION 1..10: note = Synthetic
GFTFSHYMMA

SEQ ID NO: 329 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 330 - protein, 7 aa, synthetic construct
  REGION 1..7: note = Synthetic
SQSISTW

SEQ ID NO: 331 - skipped

SEQ ID NO: 332 - protein, 6 aa, synthetic construct
  REGION 1..6: note = Synthetic
YNSYSR

SEQ ID NO: 333 - protein, 11 aa, synthetic construct
  REGION 1..11: note = Synthetic
RASQSISTWLA

SEQ ID NO: 334 - protein, 7 aa, synthetic construct
  REGION 1..7: note = Synthetic
KASNLHT

SEQ ID NO: 335 - protein, 9 aa, synthetic construct
  REGION 1..9: note = Synthetic
QQYNSYSRT

SEQ ID NO: 336 - protein, 6 aa, synthetic construct
  REGION 1..6: note = Synthetic
QSISTW

SEQ ID NO: 337 - protein, 976 aa, synthetic construct
  REGION 1..976: note = Synthetic
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 338 - protein, 105 aa, synthetic construct
  REGION 1..105: note = Synthetic
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 339 - protein, 118 aa, synthetic construct
  REGION 1..118: note = Synthetic
QVQLVQSGSELKKPGASVKVSCKASGYTFTNFRMNWVKQAPGQGLKWMGWINTYTGEPTYVDDLKGRFVFSLDTSVSTAYLQISSLKAEDTAVYYCARKGIARAMDYWGQGTTVTVSS

SEQ ID NO: 340 - protein, 113 aa, synthetic construct
  REGION 1..113: note = Synthetic
DIVMTQSPDSLAVSLGERATINCKSSQSLLDSGNQKNYLAWYQQKPGQPPKLLIFGASTRESGVPDRFSGSGSGTDFTLTISSLQAEDVAVYYCQNDHSYPYTFGQGTKLEIK

SEQ ID NO: 341 - protein, 114 aa, synthetic construct
  REGION 1..114: note = Synthetic
QVQLVQSGAEVKKPGSSVKVSCKASGYTFTSYWMHWVRQAPGQGLEWMGYINPSSGHIENNQKFKDRVTITADKSTSTAYMELSSLRSEDTAVYYCARGRFAYWGQGTLVTVSS

SEQ ID NO: 342 - protein, 108 aa, synthetic construct
  REGION 1..108: note = Synthetic
EIVLTQSPATLSLSPGERATLSCSASSSVSSGYLYWYQQKPGQAPRLLIYSTSNLASGIPARFSGSGSGTDFTLTISSLEPEDFAVYYCHQWSSYPFTFGSGTKLEIK

SEQ ID NO: 343 - protein, 118 aa, synthetic construct
  REGION 1..118: note = Synthetic
QVQLQESGPGLVKPSQTLSLTCTVSGFSLTDYGVSWIRQPPGKGLEWIGVIWGGGSTYHNSALKSRVTISVDTSKNQFSLKLSSVTAADTAVYYCAKTSYDGYYFDYWGQGTLVTVSS

SEQ ID NO: 344 - protein, 106 aa, synthetic construct
  REGION 1..106: note = Synthetic
EIVLTQSPATLSLSPGERATLSCSASSSINNMHWYQQKPGQAPRLLIYDTSKLASGIPARFSGSGSGTDFTLTISSLEPEDFAVYYCQQWSFNPLTFGQGTKLEIK

SEQ ID NO: 345 - DNA, 345 bp, synthetic construct
  misc_feature 1..345: note = Synthetic
  source 1..345: mol_type = other DNA
gaagttcagctggttcagtctggcgccgaagtgaagaaacctggcgccaccgtgaagatcagctgcaaggtgtccggctacaccttcaccgactactacatccactgggtgcagcaggcccctggcaaaggacttgagtggatgggcagagtgaaccccgatcacggcgacagctactacaaccagaagttcaaggacaaggccaccatcaccgccgacaagagcaccgataccgcctacatggaactgagcagcctgagaagcgaggataccgccgtgtacttctgcgcccggaactacctgtttgaccactggggacagggcaccctggtcacagttagttct

SEQ ID NO: 346 - DNA, 315 bp, synthetic construct
  misc_feature 1..315: note = Synthetic
  source 1..315: mol_type = other DNA
gacattcagatgacacagagccctagcagcgtgtccgcctctgtgggagacagagtgaccatcacatgcaaggccagccaggatgtgggaacagccgtggcttggtatcagcagaagcctggcaaggcccctaagctgctgatctactgggccagcatcagacacacaggcgtgcccagcagattttctggcagcggctctggcaccgacttcaccctgaccatatctagcctgcagccagaggacttcgccacctactactgccaccagtttgccacctacaccttcggcggaggcaccaaggtggaaatcaag

SEQ ID NO: 347 - DNA, 354 bp, synthetic construct
  misc_feature 1..354: note = Synthetic
  source 1..354: mol_type = other DNA
caggtgcagctgcaggaatctggacctggcctcgtgaagccctcccagaccctgtctctgacctgcaccgtgtccggcttctccctgaccgattacggcgtgtcctggatcagacagccccctggcaagggcctggaatggatcggagtgatctggggcggaggctccacctaccacaactccgccctgaagtccagagtgaccatctccgtggacacctccaagaaccagttcagcctgaagctgtcctccgtgaccgccgctgataccgccgtgtactactgcgccaagacctcctacgacggctactacttcgactactggggccagggcaccctcgtgaccgtgtcatct

SEQ ID NO: 348 - DNA, 318 bp, synthetic construct
  misc_feature 1..318: note = Synthetic
  source 1..318: mol_type = other DNA
gagatcgtgctgacccagtctcctgccaccctgtctctgagccctggcgagagagctaccctgtcctgctccgcctcctcctccatcaacaacatgcactggtatcagcagaagcccggccaggcccccagactgctgatctacgacacctccaagctggcctccggcatccctgccagattctccggctctggctctggcaccgactttaccctgaccatctccagcctggaacccgaggacttcgccgtgtactactgccagcagtggtccttcaaccccctgacctttggccagggcaccaagctggaaatcaag

SEQ ID NO: 349 - protein, 8 aa, synthetic construct
  REGION 1..8: note = Synthetic
GYTFTNFR

SEQ ID NO: 350 - protein, 8 aa, synthetic construct
  REGION 1..8: note = Synthetic
INTYTGEP

SEQ ID NO: 351 - protein, 11 aa, synthetic construct
  REGION 1..11: note = Synthetic
ARKGIARAMDY

SEQ ID NO: 352 - protein, 12 aa, synthetic construct
  REGION 1..12: note = Synthetic
QSLLDSGNQKNY

SEQ ID NO: 353 - skipped

SEQ ID NO: 354 - protein, 9 aa, synthetic construct
  REGION 1..9: note = Synthetic
QNDHSYPYT

SEQ ID NO: 355 - protein, 8 aa, synthetic construct
  REGION 1..8: note = Synthetic
GYTFTSYW

SEQ ID NO: 356 - protein, 8 aa, synthetic construct
  REGION 1..8: note = Synthetic
INPSSGHI

SEQ ID NO: 357 - protein, 7 aa, synthetic construct
  REGION 1..7: note = Synthetic
ARGRFAY

SEQ ID NO: 358 - protein, 7 aa, synthetic construct
  REGION 1..7: note = Synthetic
SSVSSGY

SEQ ID NO: 359 - skipped

SEQ ID NO: 360 - protein, 9 aa, synthetic construct
  REGION 1..9: note = Synthetic
HQWSSYPFT

SEQ ID NO: 361 - protein, 8 aa, synthetic construct
  REGION 1..8: note = Synthetic
GFSLTDYG

SEQ ID NO: 362 - protein, 7 aa, synthetic construct
  REGION 1..7: note = Synthetic
IWGGGST

SEQ ID NO: 363 - protein, 12 aa, synthetic construct
  REGION 1..12: note = Synthetic
AKTSYDGYYFDY

SEQ ID NO: 364 - protein, 4 aa, synthetic construct
  REGION 1..4: note = Synthetic
SSIN

SEQ ID NO: 365 - skipped

SEQ ID NO: 366 - protein, 9 aa, synthetic construct
  REGION 1..9: note = Synthetic
QQWSFNPLT

SEQ ID NO: 367 - protein, 447 aa, synthetic construct
  REGION 1..447: note = Synthetic
QVQLVQSGSELKKPGASVKVSCKASGYTFTNFRMNWVKQAPGQGLKWMGWINTYTGEPTYVDDLKGRFVFSLDTSVSTAYLQISSLKAEDTAVYYCARKGIARAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 368 - protein, 220 aa, synthetic construct
  REGION 1..220: note = Synthetic
DIVMTQSPDSLAVSLGERATINCKSSQSLLDSGNQKNYLAWYQQKPGQPPKLLIFGASTRESGVPDRFSGSGSGTDFTLTISSLQAEDVAVYYCQNDHSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 369 - protein, 443 aa, synthetic construct
  REGION 1..443: note = Synthetic
QVQLVQSGAEVKKPGSSVKVSCKASGYTFTSYWMHWVRQAPGQGLEWMGYINPSSGHIENNQKFKDRVTITADKSTSTAYMELSSLRSEDTAVYYCARGRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 370 - protein, 215 aa, synthetic construct
  REGION 1..215: note = Synthetic
EIVLTQSPATLSLSPGERATLSCSASSSVSSGYLYWYQQKPGQAPRLLIYSTSNLASGIPARFSGSGSGTDFTLTISSLEPEDFAVYYCHQWSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 371 - protein, 448 aa, synthetic construct
  REGION 1..448: note = Synthetic
QVQLQESGPGLVKPSQTLSLTCTVSGFSLTDYGVSWIRQPPGKGLEWIGVIWGGGSTYHNSALKSRVTISVDTSKNQFSLKLSSVTAADTAVYYCAKTSYDGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 213 aa, synthetic construct
  REGION 1..213: note = Synthetic
EIVLTQSPATLSLSPGERATLSCSASSSINNMHWYQQKPGQAPRLLIYDTSKLASGIPARFSGSGSGTDFTLTISSLEPEDFAVYYCQQWSFNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 373 - protein, 444 aa, synthetic construct
  REGION 1..444: note = Synthetic
QVQLVQSGSELKKPGASVKVSCKASGYTFTNFRMNWVKQAPGQGLKWMGWINTYTGEPTYVDDLKGRFVFSLDTSVSTAYLQISSLKAEDTAVYYCARKGIARAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 374 - protein, 219 aa, synthetic construct
  REGION 1..219: note = Synthetic
DIVMTQSPDSLAVSLGERATINCKSSQSLLDSGNQKNYLAWYQQKPGQPPKLLIFGASTRESGVPDRFSGSGSGTDFTLTISSLQAEDVAVYYCQNDHSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 440 aa, synthetic construct
  REGION 1..440: note = Synthetic
QVQLVQSGAEVKKPGSSVKVSCKASGYTFTSYWMHWVRQAPGQGLEWMGYINPSSGHIENNQKFKDRVTITADKSTSTAYMELSSLRSEDTAVYYCARGRFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 376 - protein, 444 aa, synthetic construct
  REGION 1..444: note = Synthetic
QVQLQESGPGLVKPSQTLSLTCTVSGFSLTDYGVSWIRQPPGKGLEWIGVIWGGGSTYHNSALKSRVTISVDTSKNQFSLKLSSVTAADTAVYYCAKTSYDGYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 377 - protein, 445 aa, synthetic construct
  REGION 1..445: note = Synthetic
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 378 - DNA, 1335 bp, synthetic construct
  misc_feature 1..1335: note = Synthetic
  source 1..1335: mol_type = other DNA
gaagttcagctggttcagtctggcgccgaagtgaagaaacctggcgccaccgtgaagatcagctgcaaggtgtccggctacaccttcaccgactactacatccactgggtgcagcaggcccctggcaaaggacttgagtggatgggcagagtgaaccccgatcacggcgacagctactacaaccagaagttcaaggacaaggccaccatcaccgccgacaagagcaccgataccgcctacatggaactgagcagcctgagaagcgaggataccgccgtgtacttctgcgcccggaactacctgtttgaccactggggacagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 379 - protein, 212 aa, synthetic construct
  REGION 1..212: note = Synthetic
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 380 - DNA, 636 bp, synthetic construct
  misc_feature 1..636: note = Synthetic
  source 1..636: mol_type = other DNA
gacattcagatgacacagagccctagcagcgtgtccgcctctgtgggagacagagtgaccatcacatgcaaggccagccaggatgtgggaacagccgtggcttggtatcagcagaagcctggcaaggcccctaagctgctgatctactgggccagcatcagacacacaggcgtgcccagcagattttctggcagcggctctggcaccgacttcaccctgaccatatctagcctgcagccagaggacttcgccacctactactgccaccagtttgccacctacaccttcggcggaggcaccaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 381 - protein, 449 aa, synthetic construct
  REGION 1..449: note = Synthetic
EVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 382 - DNA, 1347 bp, synthetic construct
  misc_feature 1..1347: note = Synthetic
  source 1..1347: mol_type = other DNA
gaagtgcagctggttgaatctggcggaggactggttcagcctggcggatctctgagactgtcttgtgccgccagcggctacaccttcaccaacttcggcatgaactgggtccgacaggcccctggcaaaggccttgaatgggtcgcctggatcaacaccaataccggcgagcccagatacgccgaagagttcaagggcagattcaccatcagccgggacaacgccaagaacagcctgtacctgcagatgaactccctgagagccgaggacaccgccgtgtactactgtgccagagattgggacggcgcctacttcttcgattactggggccagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 383 - protein, 449 aa, synthetic construct
  REGION 1..449: note = Synthetic
EVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 384 - DNA, 1347 bp, synthetic construct
  misc_feature 1..1347: note = Synthetic
  source 1..1347: mol_type = other DNA
gaagtgcagctggttgaatctggcggaggactggttcagcctggcggatctctgagactgtcttgtgccgccagcggctacaccttcaccaacttcggcatgaactgggtccgacaggcccctggcaaaggccttgaatgggtcgcctggatcaacaccaataccggcgagcccagatacgccgaagagttcaagggcagattcaccatcagccgggacaacgccaagaacagcctgtacctgcagatgaactccctgagagccgaggacaccgccgtgtactactgtgccagagattgggacggcgcctacttcttcgattactggggccagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 385 - protein, 214 aa, synthetic construct
  REGION 1..214: note = Synthetic
DIQMTQSPSSLSASVGDRVTITCKASQSVSNDVAWYQQKPGKAPKLLIYFATNRYTGVPSRFSGSGYGTDFTLTISSLQPEDFATYYCQQDYSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 386 - DNA, 642 bp, synthetic construct
  misc_feature 1..642: note = Synthetic
  source 1..642: mol_type = other DNA
gacattcagatgacacagagccctagcagcctgagcgcctctgtgggagacagagtgaccatcacatgcaaggccagccagagcgtgtccaacgacgtggcatggtatcagcagaagcctggcaaggcccctaagctgctgatctacttcgccaccaacagatacaccggcgtgcccagcagattttccggctctggctacggcaccgacttcaccctgaccatatctagcctgcagccagaggatttcgccacctactactgccagcaggactacagcagcccttggacatttggccagggaacaaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 387 - protein, 451 aa, synthetic construct
  REGION 1..451: note = Synthetic
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 388 - DNA, 1353 bp, synthetic construct
  misc_feature 1..1353: note = Synthetic
  source 1..1353: mol_type = other DNA
caggttcagctggttcagtctggcgccgaagtgaagaaacctggcgcctctgtgaaggtgtcctgcaaggccagcggctacacctttacaacagccggcatgcagtgggtccgacaggctcctggacaaggcttggaatggatgggctggatcaacacccacagcggcgtgcccaaatacgccgaggatttcaagggcagagtgaccatcagcgccgacaccagcacaagcacagcctacctgcagctgagcagcctgaagtctgaggacaccgccgtgtactactgtgccagaagcggctttggcagcagctactggtacttcgatgtgtggggccagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 389 - protein, 451 aa, synthetic construct
  REGION 1..451: note = Synthetic
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 390 - DNA, 1353 bp, synthetic construct
  misc_feature 1..1353: note = Synthetic
  source 1..1353: mol_type = other DNA
caggttcagctggttcagtctggcgccgaagtgaagaaacctggcgcctctgtgaaggtgtcctgcaaggccagcggctacacctttacaacagccggcatgcagtgggtccgacaggctcctggacaaggcttggaatggatgggctggatcaacacccacagcggcgtgcccaaatacgccgaggatttcaagggcagagtgaccatcagcgccgacaccagcacaagcacagcctacctgcagctgagcagcctgaagtctgaggacaccgccgtgtactactgtgccagaagcggctttggcagcagctactggtacttcgatgtgtggggccagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 391 - protein, 214 aa, synthetic construct
  REGION 1..214: note = Synthetic
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 392 - DNA, 645 bp, synthetic construct
  misc_feature 1..645: note = Synthetic
  source 1..645: mol_type = other DNA
gacattcagatgacacagagccctagcagcctgagcgcctctgtgggagacagagtgaccatcacatgcaaggccagccaggatgtgtctacagccgtggcctggtatcagcagaagcctggaaaggcccctaagctgctgatctacagcgccagctacagatacaccggcgtgcccagcagattttctggcagcggctctggcaccgacttcaccctgaccatatctagcctgcagccagaggacttcgccgtgtactactgccagcagcactacatcacccctctgacctttggccaggggaccaagctggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgctga

SEQ ID NO: 393 - protein, 10 aa, synthetic construct
  REGION 1..10: note = Synthetic
GYTFTDYYIH

SEQ ID NO: 394 - protein, 17 aa, synthetic construct
  REGION 1..17: note = Synthetic
RVNPDHGDSYYNQKFKD

SEQ ID NO: 395 - protein, 6 aa, synthetic construct
  REGION 1..6: note = Synthetic
NYLFDH

SEQ ID NO: 396 - protein, 5 aa, synthetic construct
  REGION 1..5: note = Synthetic
DYYIH

SEQ ID NO: 397 - protein, 7 aa, synthetic construct
  REGION 1..7: note = Synthetic
GYTFTDY

SEQ ID NO: 398 - protein, 6 aa, synthetic construct
  REGION 1..6: note = Synthetic
NPDHGD

SEQ ID NO: 399 - protein, 8 aa, synthetic construct
  REGION 1..8: note = Synthetic
GYTFTDYY

SEQ ID NO: 400 - protein, 8 aa, synthetic construct
  REGION 1..8: note = Synthetic
VNPDHGDS

SEQ ID NO: 401 - protein, 8 aa, synthetic construct
  REGION 1..8: note = Synthetic
ARNYLFDH

SEQ ID NO: 402 - protein, 11 aa, synthetic construct
  REGION 1..11: note = Synthetic
KASQDVGTAVA

SEQ ID NO: 403 - protein, 7 aa, synthetic construct
  REGION 1..7: note = Synthetic
WASIRHT

SEQ ID NO: 404 - protein, 7 aa, synthetic construct
  REGION 1..7: note = Synthetic
HQFATYT

SEQ ID NO: 405 - protein, 7 aa, synthetic construct
  REGION 1..7: note = Synthetic
SQDVGTA

SEQ ID NO: 406 - protein, 4 aa, synthetic construct
  REGION 1..4: note = Synthetic
FATY

SEQ ID NO: 407 - protein, 6 aa, synthetic construct
  REGION 1..6: note = Synthetic
QDVGTA

SEQ ID NO: 408 - protein, 10 aa, synthetic construct
  REGION 1..10: note = Synthetic
GYTFTNFGMN

SEQ ID NO: 409 - protein, 17 aa, synthetic construct
  REGION 1..17: note = Synthetic
WINTNTGEPRYAEEFKG

SEQ ID NO: 410 - protein, 10 aa, synthetic construct
  REGION 1..10: note = Synthetic
DWDGAYFFDY

SEQ ID NO: 411 - protein, 7 aa, synthetic construct
  REGION 1..7: note = Synthetic
GYTFTNF

SEQ ID NO: 412 - protein, 6 aa, synthetic construct
  REGION 1..6: note = Synthetic
NTNTGE

SEQ ID NO: 413 - protein, 5 aa, synthetic construct
  REGION 1..5: note = Synthetic
NFGMN

SEQ ID NO: 414 - protein, 8 aa, synthetic construct
  REGION 1..8: note = Synthetic
INTNTGEP

SEQ ID NO: 415 - protein, 12 aa, synthetic construct
  REGION 1..12: note = Synthetic
ARDWDGAYFFDY

SEQ ID NO: 416 - protein, 11 aa, synthetic construct
  REGION 1..11: note = Synthetic
KASQSVSNDVA

SEQ ID NO: 417 - protein, 7 aa, synthetic construct
  REGION 1..7: note = Synthetic
FATNRYT

SEQ ID NO: 418 - protein, 9 aa, synthetic construct
  REGION 1..9: note = Synthetic
QQDYSSPWT

SEQ ID NO: 419 - protein, 7 aa, synthetic construct
  REGION 1..7: note = Synthetic
SQSVSND

SEQ ID NO: 420 - skipped

SEQ ID NO: 421 - protein, 6 aa, synthetic construct
  REGION 1..6: note = Synthetic
DYSSPW

SEQ ID NO: 422 - protein, 6 aa, synthetic construct
  REGION 1..6: note = Synthetic
QSVSND

SEQ ID NO: 423 - protein, 10 aa, synthetic construct
  REGION 1..10: note = Synthetic
GYTFTTAGMQ

SEQ ID NO: 424 - protein, 17 aa, synthetic construct
  REGION 1..17: note = Synthetic
WINTHSGVPKYAEDFKG

SEQ ID NO: 425 - protein, 12 aa, synthetic construct
  REGION 1..12: note = Synthetic
SGFGSSYWYFDV

SEQ ID NO: 426 - protein, 7 aa, synthetic construct
  REGION 1..7: note = Synthetic
GYTFTTA

SEQ ID NO: 427 - protein, 6 aa, synthetic construct
  REGION 1..6: note = Synthetic
NTHSGV

SEQ ID NO: 428 - protein, 5 aa, synthetic construct
  REGION 1..5: note = Synthetic
TAGMQ

SEQ ID NO: 429 - protein, 8 aa, synthetic construct
  REGION 1..8: note = Synthetic
INTHSGVP

SEQ ID NO: 430 - protein, 14 aa, synthetic construct
  REGION 1..14: note = Synthetic
ARSGFGSSYWYFDV

SEQ ID NO: 431 - protein, 11 aa, synthetic construct
  REGION 1..11: note = Synthetic
KASQDVSTAVA

SEQ ID NO: 432 - protein, 7 aa, synthetic construct
  REGION 1..7: note = Synthetic
SASYRYT

SEQ ID NO: 433 - protein, 9 aa, synthetic construct
  REGION 1..9: note = Synthetic
QQHYITPLT

SEQ ID NO: 434 - protein, 7 aa, synthetic construct
  REGION 1..7: note = Synthetic
SQDVSTA

SEQ ID NO: 435 - protein, 6 aa, synthetic construct
  REGION 1..6: note = Synthetic
HYITPL

SEQ ID NO: 436 - protein, 6 aa, synthetic construct
  REGION 1..6: note = Synthetic
QDVSTA

SEQ ID NO: 437 - protein, 115 aa, synthetic construct
  REGION 1..115: note = Synthetic
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 438 - protein, 8 aa, synthetic construct
  REGION 1..8: note = Synthetic
GYTFTTAG